(19) United States
(12) Patent Application Publication
(10) Pub. No.: US 2024/0383915 A1
(43) Pub. Date: Nov. 21, 2024

(54) OXAZEPINE COMPOUNDS AND USES THEREOF IN THE TREATMENT OF CANCER

(71) Applicant: Genentech, Inc., South San Francisco, CA (US)

(72) Inventors: Lewis J. GAZZARD, Belmont, CA (US); Samantha Alyson GREEN, San Mateo, CA (US); Elizabeth H. KELLEY, Saratoga, CA (US); Matthew Leo LANDRY, San Mateo, CA (US); Sushant MALHOTRA, Burlingame, CA (US); Benjamin David RAVETZ, Burlingame, CA (US); Michael SIU, Burlingame, CA (US); Jack Alexander TERRETT, San Francisco, CA (US); BinQing WEI, San Carlos, CA (US); Steven DO, San Jose, CA (US); Yun-Xing CHENG, Beijing (CN); Limin CHENG, Beijing (CN); Jianfeng XIN, Beijing (CN); Mingtao HE, Beijing (CN); Guosheng WU, Beijing (CN); Yinlei SUN, Beijing (CN); Cheng SHAO, Beijing (CN); Aijun LU, Beijing (CN); Yulai ZHANG, Beijing (CN)

(73) Assignee: Genentech, Inc., South San Francisco, CA (US)

(21) Appl. No.: 18/554,438

(22) PCT Filed: Apr. 6, 2022
(86) PCT No.: PCT/US2022/023573

§ 371 Date: Oct. 6, 2023

(30) Foreign Application Priority Data
Apr. 8, 2021 (WO) PCT/CN2021/085959

(51) Int. Cl. C07D 498/04; A61K 31/553; A61K 45/06; C07D 519/00

(52) U.S. Cl. CPC C07D 498/04; A61K 31/553; A61K 45/06; C07D 519/00

(57) ABSTRACT

Provided herein are acyclic oxazepinyl compounds useful in the treatment on cancers.

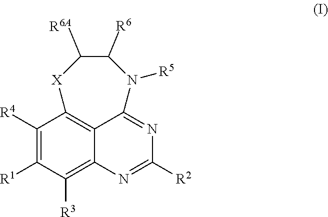

### CROSS REFERENCE TO RELATED APPLICATIONS

This International Patent Application claims the benefit of International Patent Application Number PCT/CN2021/085959, filed 8 Apr. 2021, which is incorporated herein by reference in its entirety and for all purposes.

### FIELD OF INVENTION

Provided herein are acyclic compounds useful in the treatment of cancers comprising a KRas mutation, compositions of such compounds, and methods of treating cancers comprising a KRas mutation.

### BACKGROUND

Ras is a small GTP-binding protein that functions as a nucleotide-dependent switch for central growth signaling pathways. In response to extracellular signals, Ras is converted from a GDP-bound (Ras^{GDP}) to a GTP-bound (Ras^{GTP}) state, as catalyzed by guanine nucleotide exchange factors (GEFs), notably the SOS1 protein. Active Ras^{GTP} mediates its diverse growth-stimulating functions through its direct interactions with effectors including Raf, PI3K, and Ral guanine nucleotide dissociation stimulator. The intrinsic GTPase activity of Ras then hydrolyzes GTP to GDP to terminate Ras signaling. The Ras GTPase activity can be further accelerated by its interactions with GTPase-activating proteins (GAPs), including the neurofibromin 1 tumor suppressor.

Mutant Ras has a reduced GTPase activity, which prolongs its activated state, thereby promoting Ras-dependent signaling and cancer cell survival or growth. Mutation in Ras that affects its ability to interact with GAP or to convert GTP back to GDP will result in a prolonged activation of the protein and consequently a prolonged signal to the cell telling it to continue to grow and divide. Because these signals result in cell growth and division, overactive RAS signaling may ultimately lead to cancer. Mutations in any one of the three main isoforms of RAS (HRas, NRas, or KRas) genes are common events in human tumorigenesis. Among the three Ras isoforms (K, N, and H), KRas is most frequently mutated.

The most common KRas mutations are found at residue G12 and G13 in the P-loop and at residue Q61. Mutations of Ras in cancer are associated with poor prognosis. Inactivation of oncogenic Ras in mice results in tumor shrinkage. Thus, Ras is widely considered an oncology target of exceptional importance.

Accordingly, there is a pressing need for therapies for mutant KRas mediated cancers.

### SUMMARY

Provided herein are solutions to the problems above and other problems in the art.

In a first aspect provided herein is a compound of formula (I) or a stereoisomer, atropisomer, tautomer, or pharmaceutically acceptable salt thereof as described herein.

In another aspect provided herein is a compound of formula (II), (IIa), (IIb), (IIc), or (IId), or a stereoisomer, atropisomer, tautomer, or pharmaceutically acceptable salt thereof as described herein.

In another aspect provided herein is a compound of formula (III), (IIIa), (IIIb), (IIIc), (IIId), or a stereoisomer, atropisomer, tautomer, or pharmaceutically acceptable salt thereof as described herein.

In another aspect provided herein is a compound of formula (IV), (IVa), (IVb), or (IVc), or a stereoisomer, atropisomer, tautomer, or pharmaceutically acceptable salt thereof as described herein.

In another aspect provided herein is a compound of formula (V), (Va), (Vb), or (Vc), or a stereoisomer, atropisomer, tautomer, or pharmaceutically acceptable salt thereof as described herein.

In another aspect provided herein is a compound or pharmaceutically acceptable salt thereof as set forth in Table 1.

In another aspect provided herein is a pharmaceutical composition comprising a compound, stereoisomer, atropisomer, tautomer, or pharmaceutically acceptable salt thereof as described herein.

In another aspect provided herein is a method of treating a cancer comprising a KRas mutation, the method comprising administering to a patient having such cancer, a compound, stereoisomer, atropisomer, tautomer, or pharmaceutically acceptable salt thereof as described herein.

In another aspect provided herein is a method for regulating activity of a KRas mutant protein, the method comprising reacting the mutant protein with a compound, or stereoisomer, atropisomer, tautomer, or pharmaceutically acceptable salt thereof as described herein.

In another aspect provided herein is a method for inhibiting proliferation of a cell population, the method comprising contacting the cell population with a compound, or stereoisomer, atropisomer, tautomer, or pharmaceutically acceptable salt thereof as described herein.

In another aspect provided herein is a method for inhibiting tumor metastasis comprising administering to an individual in need thereof a therapeutically effective amount of the compound or stereoisomer, atropisomer, tautomer, or pharmaceutically acceptable salt thereof as described herein or a pharmaceutical composition as described herein to a subject in need thereof.

In another aspect provided herein is method for preparing a labeled KRas mutant protein, the method comprising reacting a KRas mutant protein with a labeled compound or stereoisomer, atropisomer, tautomer, or pharmaceutically acceptable salt thereof, as described here to result in the labeled KRas mutant protein.

In another aspect provided herein is a process for synthesizing a compound or stereoisomer, atropisomer, tautomer, or pharmaceutically acceptable salt thereof as set forth herein.

### Definitions

Disclosed herein are acyclic oxazepine compounds as described herein or a stereoisomer, atropisomer, tautomer, or pharmaceutically acceptable salt thereof and pharmaceutical compositions thereof that, in certain embodiments, are inhibitors or modulators of mutant KRas. In certain instances, such compounds and compositions are inhibitors or modulators of mutant KRas^{G12V} as provided herein. In certain instances, such compounds and compositions are inhibitors or modulators of mutant KRas (i.e. pan-KRas inhibitors) as provided herein. The compounds and compositions described herein are useful in treating diseases and disorders mediated by mutant KRas.

While the disclosure herein provides enumerated embodiments, it is understood that they are not intended to limit the compounds and methods described herein to those embodiments. On the contrary, the disclosure is intended to cover all alternatives, modifications, and equivalents that can be included within the scope of the present disclosure as defined by the claims.

Unless otherwise defined, all technical and scientific terms used herein have the same meaning as commonly understood by one of ordinary skill in the art. All publications, patent applications, patents, and other references mentioned herein are incorporated by reference in their entirety. The nomenclature used in this application is based on IUPAC systematic nomenclature, unless indicated otherwise.

The following definitions are provided to facilitate understanding of certain terms used frequently herein and are not meant to limit the scope of the present disclosure. All references referred to herein are incorporated by reference in their entirety.

The terms “halogen” and “halo” are used interchangeably and refer to F, Cl, Br or 1. Additionally, terms such as “haloalkyl,” are meant to include monohaloalkyl, polyhaloalkyl, and perhaloalkyl.

The term “alkyl” refers to a saturated linear or branched-chain monovalent hydrocarbon radical. In one example, the alkyl radical is one to eighteen carbon atoms (C₁₋₁₈). In other examples, the alkyl radical is C₁₋₁₂, C₁₋₁₀, C₁₋₈, C₁₋₆, C₁₋₅, C₁₋₄, or C₁₋₃. Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl and 1-octyl.

The term “oxo” refers to ═O.

The term “alkoxy” refers to —O-alkyl.

The terms “cyano” or “nitrile” refers to —C≡N or —CN.

The term “haloalkoxy” refers to —O-haloalkyl.

The terms “hydroxy” and “hydroxyl” refer to —OH.

The term “alkylidene” refers to linear or branched-chain monovalent hydrocarbon radical having formula ═CR′R″, where R′ and R′ can be the same or different. In one example, an alkylidene radical is 1 to 6 carbons (C₁₋₆). In another example, the alkylidene radical is C₁₋₃, C₁₋₂, or C₁. Exemplary alkylidenes include, but are not limited to, methylidene (═CH₂), ethylidene (═CHCH₃), and propylidene (═CH—CH₂—CH₃).

The term “alkenyl” refers to linear or branched-chain monovalent hydrocarbon radical with at least one carbon-carbon double bond, and includes radicals having “cis” and “trans” orientations, or alternatively, “E” and “Z” orientations. In one example, the alkenyl radical is two to eighteen carbon atoms (C₂₋₁₈). In other examples, the alkenyl radical is C₂₋₁₂, C₂₋₁₀, C₂₋₈, C₂₋₄, or C₂₋₃. Examples include, but are not limited to, ethenyl or vinyl (—CH═CH₂), prop-1-enyl (—CH═CHCH₃), prop-2-enyl (—CH₂CH═CH₂), 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl.

The term “alkynyl” refers to a linear or branched monovalent hydrocarbon radical with at least one carbon-carbon, triple bond. In one example, the alkynyl radical is two to eighteen carbon atoms (C₂₋₁₃). In other examples, the alkynyl radical is C₂₋₁₂, C₂₋₁₀, C₂-M, C₂₋₆, or C₂₋₃. Examples include, but are not limited to, ethynyl (—C═CH), prop-1-ynyl (—C═CCH₃), prop-2-ynyl (propargyl, —CH₂C═CH), but-1-ynyl, but-2-ynyl, and but-3-ynyl.

The term “alkylene” refers to a saturated, branched, or straight chain hydrocarbon group having two monovalent radical centers derived by the removal of two hydrogen atoms from the same or two different carbon atoms of a parent alkane. In one example, the divalent alkylene group is one to eighteen carbon atoms (C₁₋₁₈). In other examples, the divalent alkylene group is C₁₋₁₂, C₁₋₁₀, C₁₋₈, C₁₋₆, C₁₋₅, C₁₋₄, or C₁₋₃. Example alkylene groups include methylene (—CH₂—), 1,1-ethyl (—CH(CH₃)—), (1,2-ethyl (—CH₂CH₂—), 1,1-propyl (—CH(CH₂CH₃)—), 2,2-propyl (—C(CH₃)₂—), 1,2-propyl (—CH(CH₃)CH₂—), 1,3-propyl (—CH₂CH₂CH₂—), 1,1-dimethyleth-1,2-yl (—C(CH₃)₂CH₂—), 1,4-butyl (—CH₂CH₂CH₂CH₂—), and the like.

The term “cycloalkyl” refers to a saturated hydrocarbon ring group. Cycloalkyl encompasses mono-, bi-, tricyclic, spiro and bridged, saturated ring systems. In one example, the cycloalkyl group is 3 to 12 carbon atoms (C₃₋₁₂). In other examples, cycloalkyl is C₃₋₄, C₃₋₅, C₃₋₇, C₃₋₈, C₃₋₁₀, or C₅₋₆. In other examples, the cycloalkyl group, as a monocycle, is C₃₋₄, C₃₋₈, C₃₋₆, or C₅₋₆. In another example, the cycoalkyl group, as a bicycle, is C₇-C₁₂. In another example, the cycloalkyl group, as a spiro system, is C₅₋₁₂.

Examples of monocyclic cycloalkyl include cyclopropyl, cyclobutyl, cyclopentyl, cyclohexyl, cycloheptyl, cyclooctyl, cyclononyl, cyclodecyl, cycloundecyl and cyclododecyl. Exemplary arrangements of bicyclic cycloalkyls having 7 to 12 ring atoms include, but are not limited to, [4,4], [4,5], [5,5], [5,6] or [6,6] ring systems. Exemplary bridged bicyclic cycloalkyls include, but are not limited to, bicyclo[2.2.1]heptane, bicyclo[2.2.2]octane and bicyclo[3.2.2]nonane. Examples of spirocycloalkyl include, spiro[2.2]pentane, spiro[2.3]hexane, spiro[2.4]heptane, spiro[2.5]octane and spiro[4.5]decane.

The terms “heterocyclic group”, “heterocyclic”, “heterocycle”, “heterocyclyl”, or “heterocyclo” are used interchangeably and refer to any mono-, bi-, tricyclic, spiro or bridged, saturated, partially saturated or unsaturated, non-aromatic ring system, having 3 to 20 ring atoms, where the ring atoms are carbon, and at least one atom in the ring or ring system is a heteroatom selected from nitrogen, sulfur or oxygen. If any ring atom of a cyclic system is a heteroatom, that system is a heterocycle, regardless of the point of attachment of the cyclic system to the rest of the molecule. In one example, heterocyclyl includes 3-10 ring atoms (“members”) and includes monocycles, bicycles, tricycles, spiro, and bridged ring systems, wherein the ring atoms are carbon, where at least one atom in the ring or ring system is a heteroatom selected from nitrogen, sulfur or oxygen. In other examples, heterocyclyl includes 3-6, 5-9, 4-10 or 5-10 ring atoms. In one example, heterocyclyl includes 1 to 4 heteroatoms. In one example, heterocyclyl includes 1 to 3 heteroatoms. In another example, heterocyclyl includes 3- to 7-membered monocycles having 1-2, 1-3 or 1-4 heteroatoms selected from nitrogen, sulfur or oxygen. In another example, heterocyclyl includes 4- to 6-membered monocycles having 1-2, 1-3 or 1-4 heteroatoms selected from nitrogen, sulfur or oxygen. In another example, heterocyclyl includes 3-membered monocycles. In another example, heterocyclyl includes 4-membered monocycles. In another example, heterocyclyl includes 5-6 membered monocycles. In another example, heterocyclyl includes 8, 9, or 10 membered bicycles. In such examples, the heterocyclyl group can be 4,5-, 5,5-, 4,6-, 5,6-, or 6,6-fused ring system. In some embodiments, a heterocycloalkyl includes at least one nitrogen. In one example, the heterocyclyl group includes 0 to 3 double bonds. Any nitrogen or sulfur heteroatom may optionally be oxidized (e.g., NO, SO, SO₂), and any nitrogen heteroatom may optionally be quaternized (e.g., [NR⁴]⁺Cl⁻, [NR⁴]⁺OH⁻). Example heterocycles are oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydrofuranyl, dihydrothienyl, tetrahydrothienyl, imidazolidinyl, piperidinyl, piperazinyl, isoquinolinyl, tetrahydroisoquinolinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, 1,1-dioxoisothiazolyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, thiazinyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-onyl, azaspiro[5.5]undecanyl, tetrahydroindolyl, octahydroindolyl, tetrahydroisoindolyl, tetrahydroindazolyl, 1,1-dioxohexahydrothiopyranyl.

“Aryl” as used herein refers to an unsaturated aromatic carbocyclic group having a single ring (e.g., phenyl) or multiple fused or spiro rings (e.g., naphthyl or anthryl) which fused or spiro rings can or can not be aromatic. Particular aryl groups are those having from 6 to 14 annular (i.e., ring) carbon atoms (a “C₆₋₁₄ aryl”). Preferred aryl groups include those having 5 to 6 ring carbons. An aryl group having more than one ring where at least one ring is non-aromatic can be connected to the parent structure at either an aromatic ring position or at a non-aromatic ring position. In one variation, an aryl group having more than one ring where at least one ring is non-aromatic is connected to the parent structure at an aromatic ring position.

The term “heteroaryl” refers to any mono- or bicyclic aromatic ring system containing from 1 to 4 heteroatoms selected from nitrogen, oxygen, and sulfur, and in an example embodiment, at least one heteroatom is nitrogen. Included are any bicyclic groups where any of the above heteroaryl rings are fused to an aryl ring, wherein the aryl ring or the heteroaryl ring is joined to the remainder of the molecule. A heteroaryl group can have a single ring (e.g., pyridyl, furyl) or multiple fused or spiro rings (e.g., indolizinyl, benzothienyl) which fused or spiro rings can or can not be aromatic. In one embodiment, heteroaryl includes 5-6 membered monocyclic aromatic groups where one or more ring atoms is nitrogen, sulfur or oxygen. Example heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, pyrazinyl, pyridazinyl, triazinyl, tetrazinyl, tetrazolo[1,5-b]pyridazinyl, imidazol[1,2-a]pyrimidinyl and purinyl, as well as benzo-fused derivatives, for example benzoxazolyl, benzofuryl, benzothiazolyl, benzothiadiazolyl, benzotriazolyl, benzoimidazolyl, indazolyl and indolyl.

In particular embodiments, a heterocyclyl group or a heteroaryl group is attached at a carbon atom of the heterocycyl group or the heteroaryl group. By way of example, carbon bonded heterocyclyl groups include bonding arrangements at position 2, 3, 4, 5, or 6 of a pyridine ring, position 3, 4, 5, or 6 of a pyridazine ring, position 2, 4, 5, or 6 of a pyrimidine ring, position 2, 3, 5, or 6 of a pyrazine ring, position 2, 3, 4, or 5 of a furan, tetrahydrofuran, thiofuran, thiophene, pyrrole or tetrahydropyrrole ring, position 2, 4, or 5 of an oxazole, imidazole or thiazole ring, position 3, 4, or 5 of an isoxazole, pyrazole, or isothiazole ring, position 2 or 3 of an aziridine ring, position 2, 3, or 4 of an azetidine ring, position 2, 3, 4, 5, 6, 7, or 8 of a quinoline ring or position 1, 3, 4, 5, 6, 7, or 8 of an isoquinoline ring.

In certain embodiments, the heterocyclyl group or heteroaryl group is N-attached. By way of example, nitrogen bonded heterocyclyl or heteroaryl groups include bonding arrangements at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or R-carboline.

“Fused” refers to any ring structure described herein that shares one or more atoms (e.g., carbon or nitrogen atoms) with an existing ring structure in the compounds described herein.

The term “acyl” refers to a carbonyl containing substituent represented by the formula —C(═O)—R in which R is a substituent such as hydrogen, alkyl, cycloalkyl, aryl or heterocyclyl, wherein the alkyl, cycloalkyl, aryl and heterocyclyl are as defined herein. Acyl groups include alkanoyl (e.g., acetyl), aroyl (e.g., benzoyl), and heteroaroyl (e.g., pyridinoyl).

The term “haloalkyl” refers to an alkyl chain in which one or more hydrogen has been replaced by a halogen. Examples of haloalkyls are trifluoromethyl, difluoromethyl, and fluoromethyl. A substituted haloalkyl refers to a haloalkyl having a moiety other than a halogen.

As used herein a wavy line “ [symbol] ” that intersects a bond in a chemical structure indicate the point of attachment of the atom to which the wavy bond is connected in the chemical structure to the remainder of a molecule, or to the remainder of a fragment of a molecule.

In certain embodiments, divalent groups are described generically without specific bonding configurations. It is understood that the generic description is meant to include both bonding configurations, unless specified otherwise. For example, in the group R¹—R²—R³, if the group R² is described as —CH₂C(O)—, then it is understood that this group can be bonded both as R¹—CH₂C(O)—R³, and as R¹—C(O)CH₂—R³, unless specified otherwise.

The term “pharmaceutically acceptable” refers to molecular entities and compositions that do not produce an adverse, allergic or other untoward reaction when administered to an animal, such as, for example, a human, as appropriate.

Compounds described herein may be in the form of a salt, such as a pharmaceutically acceptable salt. “Pharmaceutically acceptable salts” include both acid and base addition salts. “Pharmaceutically acceptable acid addition salt” refers to those salts which retain the biological effectiveness and properties of the free bases and which are not biologically or otherwise undesirable, formed with inorganic acids such as hydrochloric acid, hydrobromic acid, sulfuric acid, nitric acid, carbonic acid, phosphoric acid and the like, and organic acids may be selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, malonic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, benzenesulfonic acid, p-toluenesulfonic acid, salicylic acid and the like.

The term “pharmaceutically acceptable base addition salts” include those derived from inorganic bases such as sodium, potassium, lithium, ammonium, calcium, magnesium, iron, zinc, copper, manganese, aluminum salts and the like. Particular base addition salts are the ammonium, potassium, sodium, calcium and magnesium salts. Salts derived from pharmaceutically acceptable organic nontoxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particular organic non-toxic bases include isopropylamine, diethylamine, ethanolamine, tromethamine, dicyclohexylamine, choline, and caffeine.

In some embodiments, a salt is selected from a hydrochloride, hydrobromide, trifluoroacetate, sulfate, phosphate, acetate, fumarate, maleate, tartrate, lactate, citrate, pyruvate, succinate, oxalate, methanesulfonate, p-toluenesulfonate, bisulfate, benzenesulfonate, ethanesulfonate, malonate, xinafoate, ascorbate, oleate, nicotinate, saccharinate, adipate, formate, glycolate, palmitate, L-lactate, D-lactate, aspartate, malate, L-tartrate, D-tartrate, stearate, furoate (e.g., 2-furoate or 3-furoate), napadisylate (naphthalene-1,5-disulfonate or naphthalene-1-(sulfonic acid)-5-sulfonate), edisylate (ethane-1,2-disulfonate or ethane-1-(sulfonic acid)-2-sulfonate), isothionate (2-hydroxyethylsulfonate), 2-mesitylenesulfonate, 2-naphthalenesulfonate, 2,5-dichlorobenzenesulfonate, D-mandelate, L-mandelate, cinnamate, benzoate, adipate, esylate, malonate, mesitylate (2-mesitylenesulfonate), napsylate (2-naphthalenesulfonate), camsylate (camphor-10-sulfonate, for example (1S)-(+)-10-camphorsulfonic acid salt), glutamate, glutarate, hippurate (2-(benzoylamino)acetate), orotate, xylate (p-xylene-2-sulfonate), and pamoic (2,2′-dihydroxy-1,1′-dinaphthylmethane-3,3′-dicarboxylate).

A “sterile” formulation is aseptic or free from all living microorganisms and their spores.

The term “stereoisomers” refer to compounds that have identical chemical constitution, but differ with regard to the arrangement of the atoms or groups in space.

Stereoisomers include diastereomers, enantiomers, atropisomers, conformers and the like.

The term “chiral” refers to molecules that have the property of non-superimposability of the mirror image partner, while the term “achiral” refers to molecules which are superimposable on their mirror image partner.

The term “diastereomer” refers to a stereoisomer with two or more centers of chirality and whose molecules are not mirror images of one another. Diastereomers have different physical properties, e.g., melting points, boiling points, spectral properties or biological activities. Mixtures of diastereomers may separate under high resolution analytical procedures such as electrophoresis and chromatography such as HPLC.

The term “enantiomers” refers to two stereoisomers of a compound that are non-superimposable mirror images of one another.

The term “atropisomers” refers to two conformers resulting from hindered rotation about a single bond where the steric strain barrier to rotation can be high enough to allow for the isolation of the each conformer.

Stereochemical definitions and conventions used herein generally follow S. P.

Parker, Ed., McGraw-Hill Dictionary of Chemical Terms (1984) McGraw-Hill Book Company, New York; and Eliel, E. and Wilen, S., “Stereochemistry of Organic Compounds”, John Wiley & Sons, Inc., New York, 1994. Many organic compounds exist in optically active forms, i.e., they have the ability to rotate the plane of plane-polarized light. In describing an optically active compound, the prefixes D and L, or R and S, are used to denote the absolute configuration of the molecule about its chiral center(s). The prefixes d and l or (+) and (−) are employed to designate the sign of rotation of plane-polarized light by the compound, with (−) or 1 meaning that the compound is levorotatory. A compound prefixed with (+) or d is dextrorotatory. For a given chemical structure, these stereoisomers are identical except that they are mirror images of one another. A specific stereoisomer may also be referred to as an enantiomer, and a mixture of such isomers is often called an enantiomeric mixture. A 50:50 mixture of enantiomers is referred to as a racemic mixture or a racemate, which may occur where there has been no stereoselection or stereospecificity in a chemical reaction or process. The terms “racemic mixture” and “racemate” refer to an equimolar mixture of two enantiomeric species, devoid of optical activity.

The term “tautomer” or “tautomeric form” refers to structural isomers of different energies that are interconvertible via a low energy barrier. For example, proton tautomers (also known as prototropic tautomers) include interconversions via migration of a proton, such as keto-enol and imine-enamine isomerizations. Valence tautomers include interconversions by reorganization of some of the bonding electrons.

Certain compounds described herein can exist in unsolvated forms as well as solvated forms, including hydrated forms. A “solvate” refers to an association or complex of one or more solvent molecules and a compound described herein. Examples of solvents that form solvates include water, isopropanol, ethanol, methanol, DMSO, ethyl acetate, acetic acid, and ethanolamine. Certain compounds described herein can exist in multiple crystalline or amorphous forms. In general, all physical forms are contemplated herein. The term “hydrate” refers to the complex where the solvent molecule is water.

The compounds and pharmaceutically acceptable salts thereof described herein also embrace isotopically-labeled compounds that are identical to those recited herein, but for the fact that one or more atoms are replaced by an atom having an atomic mass or mass number different from the atomic mass or mass number usually found in nature.

All isotopes of any particular atom or element as specified are contemplated herein, and their uses. Exemplary isotopes that can be incorporated into compounds and pharmaceutically acceptable salts thereof described herein include isotopes of hydrogen, carbon, nitrogen, oxygen, phosphorus, sulfur, fluorine, chlorine and iodine, such as ²H, ³H, ¹¹C, ¹³C, ¹⁴C, ¹³N, ¹⁵N, ¹⁵O, ¹⁷O, ¹⁸O, ³²P, ³³P, ³⁵S, ¹⁸F, ³⁶Cl, ¹²³I, and ¹²⁵I. Certain isotopically-labeled compounds or pharmaceutical acceptable salts thereof described herein (e.g., those labeled with ³H and ¹⁴C) are useful in compound and/or substrate tissue distribution assays. Tritiated (³H) and carbon-14 (¹⁴C) isotopes are useful for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Positron emitting isotopes such as ¹⁵O, ¹³N, ¹¹C and ¹⁸F are useful for positron emission tomography (PET) studies to examine substrate receptor occupancy. Isotopically labeled compounds or pharmaceutical acceptable salts thereof described herein can generally be prepared by following procedures analogous to those disclosed in the Examples herein below, by substituting an isotopically labeled reagent for a non-isotopically labeled reagent.

The term “amino-protecting group” as used herein refers to a derivative of the groups commonly employed to block or protect an amino group while reactions are carried out on other functional groups on the compound. Examples of such protecting groups include carbamates, amides, alkyl and aryl groups, and imines, as well as many N-heteroatom derivatives that can be removed to regenerate the desired amine group. Particular amino protecting groups are Pmb (p-methoxybenzyl), Boc (tert-butyloxycarbonyl), Fmoc (9-fluorenylmethyloxycarbonyl) and Cbz (carbobenzyloxy). Further examples of these groups are found in T. W. Greene and P. G. M. Wuts, “Protecting Groups in Organic Synthesis, 3^{rd} ed., John Wiley & Sons, Inc., 1999. The term “protected amino” refers to an amino group substituted with one of the above amino-protecting groups.

The term “carboxy-protecting group” as used herein refers to those groups that are stable to the conditions of subsequent reaction(s) at other positions of the molecule, which may be removed at the appropriate point without disrupting the remainder of the molecule, to give the unprotected carboxy-group. Examples of carboxy protecting groups include, ester groups and heterocyclyl groups. Ester derivatives of the carboxylic acid group may be employed to block or protect the carboxylic acid group while reactions are carried out on other functional groups on the compound. Examples of such ester groups include substituted arylalkyl, including substituted benzyls, such as 4-nitrobenzyl, 4-methoxybenzyl, 3,4-dimethoxybenzyl, 2,4-dimethoxybenzyl, 2,4,6-trimethoxybenzyl, 2,4,6-trimethylbenzyl, pentamethylbenzyl, 3,4-methylenedioxybenzyl, benzhydryl, 4,4′-dimethoxybenzhydryl, 2,2′,4,4′-tetramethoxybenzhydryl, alkyl or substituted alkyl esters such as methyl, ethyl, t-butyl allyl or t-amyl, triphenylmethyl (trityl), 4-methoxytrityl, 4,4′-dimethoxytrityl, 4,4′,4″-trimethoxytrityl, 2-phenylprop-2-yl, thioesters such as t-butyl thioester, silyl esters such as trimethylsilyl, t-butyldimethylsilyl esters, phenacyl, 2,2,2-trichloroethyl, beta-(trimethylsilyl)ethyl, beta-(di(n-butyl)methylsilyl)ethyl, p-toluenesulfonylethyl, 4-nitrobenzylsulfonylethyl, allyl, cinnamyl, 1-(trimethylsilylmethyl)prop-1-en-3-yl, and like moieties. Another example of carboxy-protecting groups are heterocyclyl groups such as 1,3-oxazolinyl. Further examples of these groups are found in T. W. Greene and P. G. M. Wuts, “Protecting Groups in Organic Synthesis, 3^{rd} ed., John Wiley & Sons, Inc., 1999. The term “protected carboxy” refers to a carboxy group substituted with one of the above carboxy-protecting groups.

Compounds and pharmaceutically acceptable salts thereof described herein may contain one or more asymmetric carbon atoms. Accordingly, the compounds may exist as diastereomers, enantiomers or mixtures thereof. The syntheses of the compounds may employ racemates, diastereomers or enantiomers as starting materials or as intermediates. Mixtures of particular diastereomeric compounds may be separated, or enriched in one or more particular diastereomers, by chromatographic or crystallization methods. Similarly, enantiomeric mixtures may be separated, or enantiomerically enriched, using the same techniques or others known in the art. Each of the asymmetric carbon or nitrogen atoms may be in the R or S configuration and both of these configurations are contemplated herein.

In the structures shown herein, where the stereochemistry of any particular chiral atom is not specified, then all stereoisomers are contemplated and included. Where stereochemistry is specified by a solid wedge or dashed line representing a particular configuration, then that stereoisomer is so specified and defined. Unless otherwise specified, if solid wedges or dashed lines are used, relative stereochemistry is intended.

A “subject,” “individual,” or “patient” is a vertebrate and are used interchangeably herein. In certain embodiments, the vertebrate is a mammal. Mammals include, but are not limited to, farm animals (such as cows), sport animals, pets (such as guinea pigs, cats, dogs, rabbits and horses), primates, mice and rats. In certain embodiments, a mammal is a human. In embodiments comprising administration of a compound of to a patient, the patient is typically in need thereof.

The terms “inhibiting” and “reducing,” or any variation of these terms, includes any measurable decrease or complete inhibition to achieve a desired result. For example, there may be a decrease of about, at most about, or at least about 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal.

The term “treatment” refers to clinical intervention designed to alter the natural course of the patient or cell being treated during the course of clinical pathology. Desirable effects of treatment include decreasing the rate of disease progression, ameliorating or palliating the disease state, and remission or improved prognosis. For example, a patient is successfully “treated” if one or more symptoms associated with a cancer described herein are mitigated or eliminated, including, but are not limited to, reducing the proliferation of (or destroying) cancerous cells, decreasing symptoms resulting from the disease, increasing the quality of life of those suffering from the disease, decreasing the dose of other medications required to treat the disease, and/or prolonging survival of patients.

The term “delaying progression” of a disease refers to deferring, hindering, slowing, retarding, stabilizing, and/or postponing development of a cancer described herein. This delay can be of varying lengths of time, depending on the history of the cancer and/or patient being treated. As is evident to one skilled in the art, a sufficient or significant delay can, in effect, encompass prevention, in that the patient does not develop cancer or relapse.

A “mutant KRas mediated disease” and the like refer to a disease described herein (e.g. a cancer described herein) having symptoms or requiring treatment as set forth herein that is/are wholly or partly associated with, a result of, a function of, or otherwise correlated to mutant KRas activity as described herein. In one such embodiment, the mutant KRas is KRas^{G12V}. In another embodiment, the mutant KRas is any G12 mutant (i.e. a pan-KRas inhibitor).

An “effective amount” or “therapeutically effective amount” is at least the minimum amount required to effect a measurable improvement or prevention of a cancer described herein. An effective amount herein may vary according to factors such as the disease state, age, sex, and weight of the patient, and the ability of the agent to elicit a desired response in the patient. An effective amount is also one in which any toxic or detrimental effects of the treatment are outweighed by the therapeutically beneficial effects. Beneficial or desired results include results such as eliminating or reducing the risk, lessening the severity, delaying the onset of the disease (including biochemical, histological and/or behavioral symptoms of the disease, its complications and intermediate pathological phenotypes presenting during development of the disease), decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In some embodiments, an effective amount of the drug may have the effect in reducing the number of cancer cells; reducing the tumor size; inhibiting (i.e., slow or stop) cancer cell infiltration into peripheral organs; inhibit (i.e., slow or stop) tumor metastasis; inhibiting (i.e., slow or stop) tumor growth; and/or relieving one or more of the symptoms associated with the disorder. An effective amount can be administered in one or more administrations.

An “administration period” or “cycle” refers to a period of time comprising administration of one or more compounds or pharmaceutically acceptable salts thereof described herein or an additional therapeutic agent (i.e. a chemotherapeutic agent) and an optional period of time comprising no administration of one or more of agents or compounds described herein. A “rest period” refers to a period of time where at least one of agent or compound described herein is not administered. In one embodiment, a rest period refers to a period of time where no agent or compound described herein is administered. A rest period as provided herein can in some instances include administration of an additional agent in the absence of a compound or pharmaceutically acceptable salt thereof described herein or vice versa. In such instances, administration of any agent during a rest period should not interfere or detriment administration of a compound or pharmaceutically acceptable salt thereof described herein.

A “dosing regimen” refers to a period of administration of a compound or pharmaceutically acceptable salt thereof described herein comprising one or more cycles, where each cycle can include administration of a compound or pharmaceutically acceptable salt thereof described herein at different times or in different amounts.

“QD” refers to administration of a compound or pharmaceutically acceptable salt thereof once daily.

“BID” refers to administration of a compound or pharmaceutically acceptable salt thereof twice a day.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times (i.e. sequential administration) in separate compositions, or administration in a composition in which both agents are present.

A “1L therapy” refers to the first line therapy administered to a treatment naïve cancer patient. Likewise, a 2L, 3L, and the like refer to subsequent therapies administered to a patient.

The term “package insert” is used to refer to instructions customarily included in commercial packages of therapeutic products, that contain information about the indications, usage, dosage, administration, contraindications and/or warnings concerning the use of such therapeutic products.

The terms “antagonist” and “inhibitor” are used interchangeably, and they refer to a compound having the ability to inhibit a biological function of a target protein, whether by inhibiting the activity or expression of the protein, such as a mutant form of KRas. Accordingly, the terms “antagonist” and “inhibitors” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with the development, growth, or spread of a tumor.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

The terms “cancer” and “cancerous”, “neoplasm”, and “tumor” and related terms are used interchangeably herein and refer to or describe the physiological condition in mammals that is typically characterized by unregulated cell growth. A “tumor” comprises one or more cancerous cells. Examples of cancer include carcinoma, blastoma, sarcoma, seminoma, glioblastoma, melanoma, leukemia, and myeloid or lymphoid malignancies. More particular examples of such cancers include squamous cell cancer (e.g., epithelial squamous cell cancer) and lung cancer including small-cell lung cancer, non-small cell lung cancer (“NSCLC”), adenocarcinoma of the lung and squamous carcinoma of the lung. Other cancers include skin, keratoacanthoma, follicular carcinoma, hairy cell leukemia, buccal cavity, pharynx (oral), lip, tongue, mouth, salivary gland, esophageal, larynx, hepatocellular, gastric, stomach, gastrointestinal, small intestine, large intestine, pancreatic, cervical, ovarian, liver, bladder, hepatoma, breast, colon, rectal, colorectal, genitourinary, biliary passage, thyroid, papillary, hepatic, endometrial, uterine, salivary gland, kidney or renal, prostate, testis, vulval, peritoneum, anal, penile, bone, multiple myeloma, B-cell lymphoma, diffuse large B-Cell lymphoma (DLBCL), central nervous system, brain, head and neck, Hodgkin's, and associated metastases. Other examples of neoplastic disorders include myeloproliferative disorders, such as polycythemia vera, essential thrombocytosis, myelofibrosis, such as primary myelofibrosis, and chronic myelogenous leukemia (CML).

A “chemotherapeutic agent” is an agent useful in the treatment of a given disorder, for example, cancer or inflammatory disorders. Examples of chemotherapeutic agents are well-known in the art. Additionally, chemotherapeutic agents include pharmaceutically acceptable salts, acids or derivatives of any of chemotherapeutic agents, as well as combinations of two or more of them.

It is specifically contemplated that any limitation discussed with respect to one embodiment provided herein may apply to any other embodiment provided herein. Furthermore, any compound and pharmaceutically acceptable salts thereof described herein or composition described herein may be used in any method provided herein, and any method provided herein may be used to produce or to utilize any compound and pharmaceutically acceptable salts thereof described herein or composition described herein.

Throughout this application, the term “about” is used to indicate that a value includes the standard deviation of error for the device or method being employed to determine the value.

### Compounds

Provided herein are compounds of formula (I):

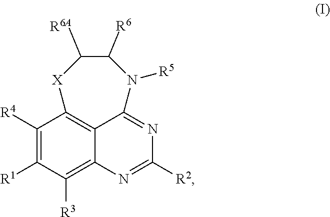

or a stereoisomer, atropisomer, tautomer, or pharmaceutically acceptable salt thereof, wherein;
X is NR¹³, O, C(R^{x})₂, C(O), SO, SO2, or S;
u is 1 or 2;
each R^{x} is independently hydrogen, halogen, unsubstituted C₁₋₃ alkyl or unsubstituted C₁₋₃ haloalkyl;
   or wherein two R^{x} together form a cyclopropyl together with the carbon to which they are bound;
R¹ is R⁷-substituted or unsubstituted indolyl, R⁷-substituted or unsubstituted benzofuranyl, R⁷-substituted or unsubstituted napthyl, R⁷-substituted or unsubstituted indazolyl, R⁷-substituted or unsubstituted indenyl, R⁷-substituted or unsubstituted benzothiazolyl, R^{7A}-substituted or unsubstituted phenyl, or R^{7A}-substituted or unsubstituted pyridinyl;
each R⁷ is independently hydrogen, halogen, CN, CH₂OH, —OH, NH₂, N(Me)₂, unsubstituted C₁₋₃ alkyl, unsubstituted C₂₋₅ alkynyl, unsubstituted C₁₋₃ haloalkyl, or unsubstituted cyclopropyl;
each R^{7A} is independently hydrogen, halogen, NH₂, N(Me)₂, unsubstituted C₁₋₃ alkyl, unsubstituted C₁₋₃ haloalkyl, or unsubstituted cyclopropyl;
R² is hydrogen, O-L¹-R⁸, R^{8A}-substituted or unsubstituted C₁₋₃ alkyl, or R^{8B}-substituted or unsubstituted 4-10 membered heterocycle;
L¹ is a bond or R^{L1}-substituted or unsubstituted C₁₋₄ alkylene;
R^{L1} is halogen or unsubstituted C₁₋₃ alkyl;
R⁸ is R⁹-substituted or unsubstituted 4-10 membered heterocycle comprising N, S, or O;
each R⁹ is independently halogen, oxo, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle:
   or wherein two R⁹ together form a C₃₋₅ cycloalkyl or 3-5 membered heterocycle;
R¹⁰ is hydrogen or halogen;
each R^{8A} is independently R^{9A}-substituted or unsubstituted C₁₋₃ alkyl, R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle;
each R^{9A} is independently halogen, oxo, unsubstituted C₁₋₃ alkyl, unsubstituted C₁₋₃ haloalkyl, unsubstituted C₁₋₃ alkoxy, unsubstituted C₁₋₃ alkylidene, R⁹-substituted or unsubstituted C₃₋₄ cycloalkyl, or R⁹-substituted or unsubstituted 4-10 membered heterocycle comprising N, S, or O;
R^{8B} is independently halogen, oxo, —NH₂, unsubstituted C₁₋₃ alkyl, unsubstituted C₁₋₃ haloalkyl, unsubstituted C₁₋₃ alkoxy, or unsubstituted C₁₋₃ alkylidene;
R³ and R⁴ are each independently hydrogen, —CN, halogen, unsubstituted C₁₋₃ alkyl, or unsubstituted cyclopropyl;
R⁵ is R^{5A}-substituted or unsubstituted C₁₋₆ alkyl, R^{5A}-substituted or unsubstituted C₁₋₆ haloalkyl, R^{5A}-substituted or unsubstituted C₃₋₁₀ cycloalkyl, R^{5A}-substituted or unsubstituted 3-10 membered heterocycle, or R^{5A}-substituted or unsubstituted 5-10 membered heteroaryl;
each R^{5A} is independently halogen, oxo, CN, OR¹¹, SR¹², SO₂R¹², NR¹³R¹⁴, C(O)N(R¹¹)₂, C(O)R¹¹, R^{5B}-substituted or unsubstituted C₁₋₆ alkyl, R^{5B}-substituted or unsubstituted C₁₋₆ haloalkyl, R⁵-substituted or unsubstituted C₃₋₆ cycloalkyl, R^{5B}-substituted or unsubstituted 3-6 membered heterocycle, R^{5B}-substituted or unsubstituted C₅₋₈ aryl, or R^{5B}-substituted or unsubstituted 5-9 membered heteroaryl;
   or wherein two R^{5A} together form a C₃₋₆ cycloalkyl or 3-6 membered heterocycle;
each R^{5B} is independently halogen, oxo, CN, OR¹¹, NR¹³R¹⁴, SR¹², SO₂R¹², C(O)N(R¹¹)₂, C(O)R¹¹, R^{5C}-substituted or unsubstituted C₁₋₃ alkyl, R^{5C}-substituted or unsubstituted C₁₋₃ haloalkyl, R^{5C}-substituted or unsubstituted C₃₋₆ cycloalkyl, R^{5C}-substituted or unsubstituted 3-6 membered heterocycle, R^{5C}-substituted or unsubstituted phenyl, or R^{5C}-substituted or unsubstituted 5-6 membered heteroaryl;
   or wherein two R^{5B} together form a C₃₋₆ cycloalkyl or 3-6 membered heterocycle;
each R^{5C} is independently halogen, oxo, CN, C(O)CH₃, OH, OCH₃, C(O)NH₂, CF₃, CHF₂, CH₂F, NR¹³R¹⁴, SCH₃, SO₂NH₂, SO₂CH₃, unsubstituted C₁₋₃ alkyl, unsubstituted C₁₋₃ haloalkyl, unsubstituted C₃₋₄ cycloalkyl, or unsubstituted 3-4 membered heterocycle;
each R¹¹ is independently hydrogen, unsubstituted C₁₋₃ alkyl, unsubstituted C₁₋₃ haloalkyl, unsubstituted C₃₋₄ cycloalkyl, or unsubstituted 3-4 membered heterocycle;
each R¹² is independently NH₂ or unsubstituted C₁₋₃ alkyl;
each R¹³ and R¹⁴ are independently hydrogen, C(O)N(R¹¹)₂, C(O)R¹¹, R¹⁵-substituted or unsubstituted C₁₋₆ alkyl, R¹⁵-substituted or unsubstituted C₃₋₆ cycloalkyl, or R¹⁵-substituted or unsubstituted 3-6 membered heterocycle;
each R¹⁵ is independently halogen, CN, C(O)CH₃, OH, OCH₃, CF₃, CHF₂, CH₂F, NH₂, NHCH₃, N(CH₃)₂, SO₂NH₂, SO₂CH₃, R¹⁶-substituted or unsubstituted C₁₋₃ alkyl, R¹⁶-substituted or unsubstituted C₃₋₆ cycloalkyl, R¹⁶-substituted or unsubstituted 3-6 membered heterocycle, R¹⁶-substituted or unsubstituted 5-9 membered aryl, or R¹⁶-substituted or unsubstituted 5-9 membered heteroaryl;
each R¹⁶ is independently halogen, CN, C(O)CH₃, OH, OCH₃, CF₃, CHF₂, CH₂F, NH₂, NHCH₃, N(CH₃)₂, SO₂NH₂, SO₂CH₃, R¹⁷-substituted or unsubstituted C₁₋₃ alkyl, R¹⁷-substituted or unsubstituted C₃₋₆ cycloalkyl, R¹⁷-substituted or unsubstituted 3-6 membered heterocycle, R¹⁷-substituted or unsubstituted 5-9 membered aryl, or R¹⁷-substituted or unsubstituted 5-9 membered heteroaryl;
each R¹⁷ is independently halogen, CN, C(O)CH₃, OH, OCH₃, CF₃, CHF₂, CH₂F, NH₂, NHCH₃, N(CH₃)₂, SO₂NH₂, SO₂CH₃, or unsubstituted C₁₋₃ alkyl;
R⁶ and R^{6A} are independently hydrogen, halogen, NR¹³R¹⁴, or R^{6B}-substituted or unsubstituted C₁₋₆ alkyl; and
R^{6B} is halogen, CN, OH, OCH₃, CF₃, CHF₂, CH₂F, or unsubstituted C₁₋₃ alkyl.

In one embodiment, X is O. In another embodiment, X is C(R^{x})₂, where R^{x} is as described herein. In one such embodiment, when X is C(R^{x})₂, R^{x} is independently hydrogen or methyl. In another such embodiment, when X is C(R^{x})₂, R^{x} is independently hydrogen or halogen. In another such embodiment, when X is C(R^{x})₂, R^{x} is independently methyl or halogen. In one embodiment, X is NR¹³, C(O), SO, SO₂, or S. In one embodiment, u is 1. In one embodiment, X is O and u is 1.

In one embodiment, R¹ is R⁷-substituted or unsubstituted indolyl, R⁷-substituted or unsubstituted benzofuranyl, R⁷-substituted or unsubstituted napthyl, R⁷-substituted or unsubstituted indazolyl, R⁷-substituted or unsubstituted benzothiazolyl, R^{7A}-substituted or unsubstituted phenyl, or R^{7A}-substituted or unsubstituted pyridinyl. In one embodiment, R¹ is R⁷-substituted or unsubstituted indolyl, R⁷-substituted or unsubstituted benzofuranyl. In another embodiment, R¹ is R⁷-substituted or unsubstituted napthyl, R⁷-substituted or unsubstituted indazolyl, R^{7A}-substituted or unsubstituted phenyl, or R^{7A}-substituted or unsubstituted pyridinyl. In still another embodiment, R¹ is R⁷-substituted or unsubstituted napthyl, R⁷-substituted or unsubstituted indazolyl, or R⁷-substituted or unsubstituted benzothiazolyl. In still another embodiment, R¹ is R⁷-substituted or unsubstituted napthyl or R⁷-substituted or unsubstituted indazolyl. In another embodiment, R¹ is R⁷-substituted or unsubstituted indenyl. In another embodiment, R¹ is R^{7A}-substituted or unsubstituted phenyl, or R^{7A}-substituted or unsubstituted pyridinyl. In another embodiment, R¹ is R⁷-substituted or unsubstituted phenyl, R⁷-substituted or unsubstituted indazolyl, or R⁷-substituted or unsubstituted pyridinyl.

In one such embodiment, R¹ is R⁷-substituted or unsubstituted phenyl. In another such embodiment, R¹ is R⁷-substituted or unsubstituted indazolyl. In another such embodiment, R¹ is R⁷-substituted or unsubstituted pyridinyl. In another such embodiment, R¹ is R⁷-substituted or unsubstituted indolyl.

In one preferred embodiment, R¹ has formula (A):

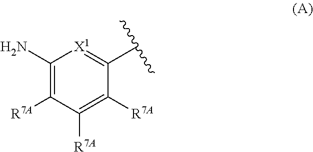

wherein X¹ is N, CH, or CF and R^{7A} is as described herein. In one such embodiment, R^{7A} is hydrogen, halogen, unsubstituted C₁₋₃ alkyl, or unsubstituted C₁₋₃ haloalkyl.

In one such embodiment, X¹ is N or CF and each R^{7A} is independently hydrogen, halogen, unsubstituted C₁₋₃ alkyl, or unsubstituted C₁₋₃ haloalkyl. In one such embodiment, R^{7A} is independently hydrogen, Cl, methyl, ethyl, or CF₃, where no more than one R^{7A} is hydrogen. In one embodiment, one R^{7A} is cyclopropyl.

In one such embodiment, the moiety of formula (A1) has formula:

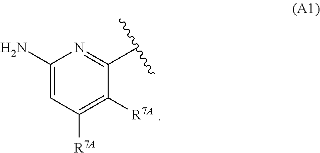

In one such embodiment, each R^{7A} is independently hydrogen, Cl, methyl, or CF₃.

In another such embodiment, each R^{7A} is independently hydrogen, methyl, or CF₃.

In one such embodiment, R¹ is

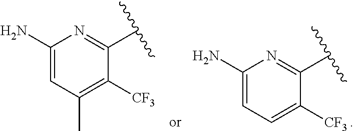 [SMILES: *c1nc(N)cc(C)c1C(F)(F)F ; *c1nc(N)ccc1C(F)(F)F]

In another such embodiment, R¹ is

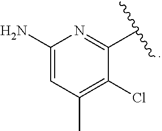 [SMILES: *c1nc(N)cc(C)c1Cl]

In one preferred embodiment, R¹ is

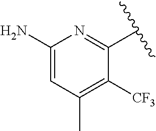 [SMILES: *c1nc(N)cc(C)c1C(F)(F)F]

In another embodiment, the moiety of formula (A) has formula:

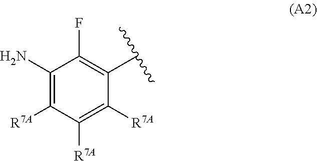

wherein R^{7A} is hydrogen, halogen, unsubstituted C₁₋₃ alkyl or unsubstituted C₁₋₃ haloalkyl. In one such embodiment, no more than one R^{7A} is hydrogen. In another such embodiment, R^{7A} is not hydrogen.

In one such embodiment, R¹ is

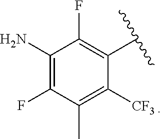 [SMILES: *c1c(F)c(N)c(F)c(C)c1C(F)(F)F]

In one such embodiment, R¹ is

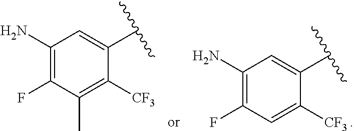 [SMILES: *c1cc(N)c(F)c(C)c1C(F)(F)F ; *c1cc(N)c(F)cc1C(F)(F)F]

In one such embodiment, R¹ is

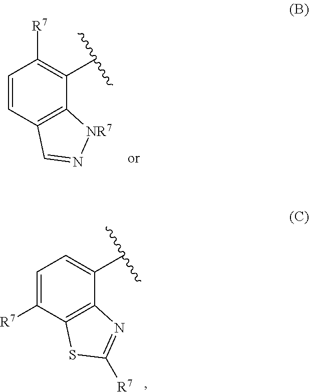

wherein each R⁷ is independently halogen, CN, NH₂, N(Me)₂, unsubstituted C₁₋₃ alkyl, unsubstituted C₂₋₃ alkynyl.

In one embodiment, R¹ is

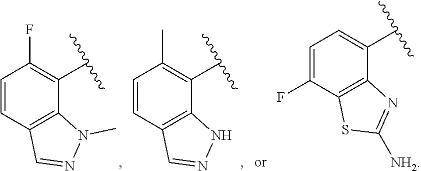

In another embodiment, R¹ is

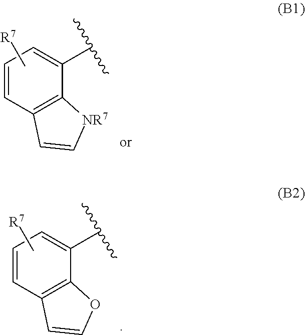

In another embodiment, R¹ is

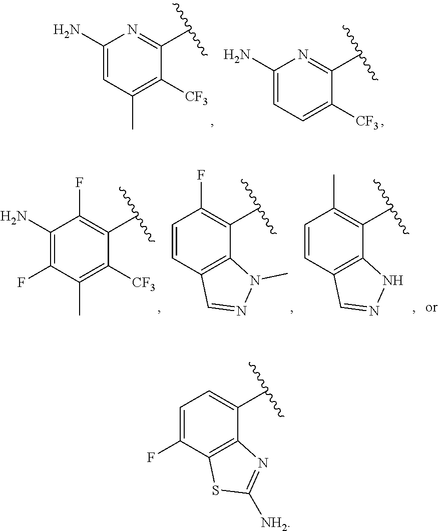

In another embodiment, R¹ is:

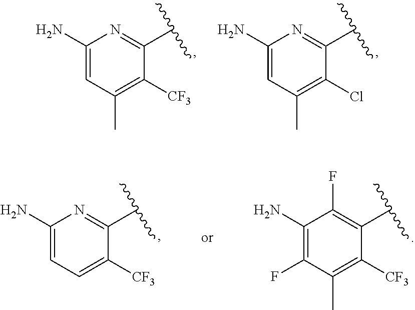

In another embodiment, R¹ is:

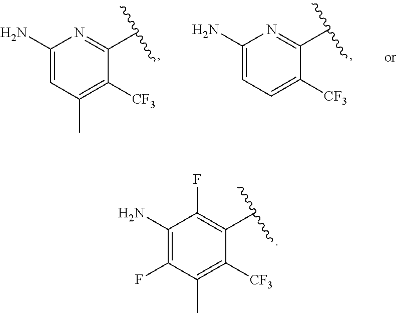 [SMILES: *c1nc(N)cc(C)c1C(F)(F)F ; *c1nc(N)ccc1C(F)(F)F ; *c1c(F)c(N)c(F)c(C)c1C(F)(F)F]

In one embodiment, R⁷ is independently hydrogen, halogen, —OH, NH₂, N(Me)₂, unsubstituted C₁₋₃ alkyl, or unsubstituted C₁₋₃ haloalkyl. In one embodiment, R⁷ is independently hydrogen, halogen, —OH, NH₂, N(Me)₂, unsubstituted C₁₋₃ alkyl, or unsubstituted C₂₋₃ alkynyl. In one embodiment, R⁷ is independently hydrogen, halogen, —CN, OH, NH₂, N(Me)₂, unsubstituted C₁₋₃ alkyl, or unsubstituted C₁₋₃ haloalkyl. In another embodiment, R⁷ is independently halogen, NH₂, or unsubstituted C₁₋₃ alkyl, or unsubstituted C₁₋₃ haloalkyl. In one embodiment of the compounds or a stereoisomer, atropisomer, tautomer, or pharmaceutically acceptable salt thereof described herein, R⁷ is not —OH.

In one embodiment, R¹ is a moiety of formula (B) or (C) where R⁷ is independently hydrogen, halogen, or unsubstituted C₁₋₃ alkyl. In one such embodiment, R⁷ is independently hydrogen or unsubstituted C₁₋₃ alkyl (e.g. methyl). In another such embodiment, R⁷ is independently halogen (e.g. F) or unsubstituted C₁₋₃ alkyl (e.g. methyl).

In one embodiment, R¹ is a moiety of formula (B) where R⁷ is independently hydrogen, halogen, —OH, NH₂, N(Me)₂, or unsubstituted C₁₋₃ alkyl. In one embodiment, R¹ is a moiety of formula (C) where R⁷ is independently hydrogen, halogen, NH₂, N(Me)₂, or unsubstituted C₁₋₃ alkyl. In one such embodiment, R⁷ is independently halogen or NH₂.

In one embodiment, R² is hydrogen or O-L¹-R⁸. In another embodiment, R² is R^{5A}-substituted or unsubstituted C₁₋₃ alkyl or R^{8B}-substituted or unsubstituted 4-10 membered heterocycle. In another embodiment, R² is R^{8B}-substituted or unsubstituted 4-6 membered heterocycle. In still another embodiment, R² is O-L¹-R⁸, R^{8A}-substituted or unsubstituted C₁₋₃ alkyl, or R^{8B}-substituted or unsubstituted 4-6 membered heterocycle comprising one nitrogen heteroatom.

In one embodiment, R² is hydrogen.

In one embodiment, the compound of formula (I) has formula:

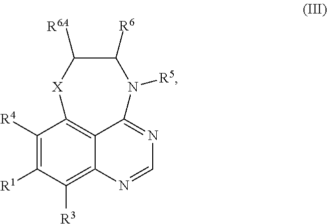

or a stereoisomer, atropisomer, tautomer, or pharmaceutically acceptable salt thereof, where R¹, R³, R⁴, R⁵, R⁶, R^{6A}, and X are as described herein.

In one such embodiment, the compound of formula (III) has formula:

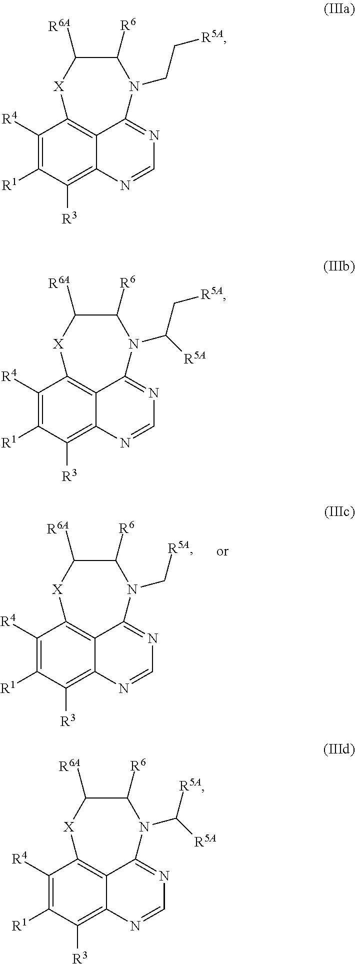

or a stereoisomer, atropisomer, tautomer, or pharmaceutically acceptable salt thereof, where R¹, R³, R⁴, R⁵, R⁶, R^{6A}, and X are as described herein.

In one embodiment, R² is O-L¹-R⁸. In one embodiment, L¹ is a bond. In one embodiment, L¹ is unsubstituted C₁₋₃ alkylene. In one preferred embodiment where R² is O-L¹-Ra. L¹ is methylene. In one such embodiment, R⁸ is R⁹-substituted or unsubstituted 4-10 membered heterocycle comprising N, S, or O.

In one embodiment, the compound of formula (I) has formula:

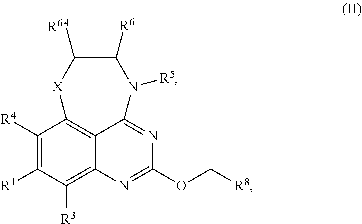

or a stereoisomer, atropisomer, tautomer, or pharmaceutically acceptable salt thereof, where R¹, R³, R⁴, R⁵, R⁶, R^{6A}, R⁸, and X are as described herein.

In one such embodiment, the compound of formula (II) has formula:

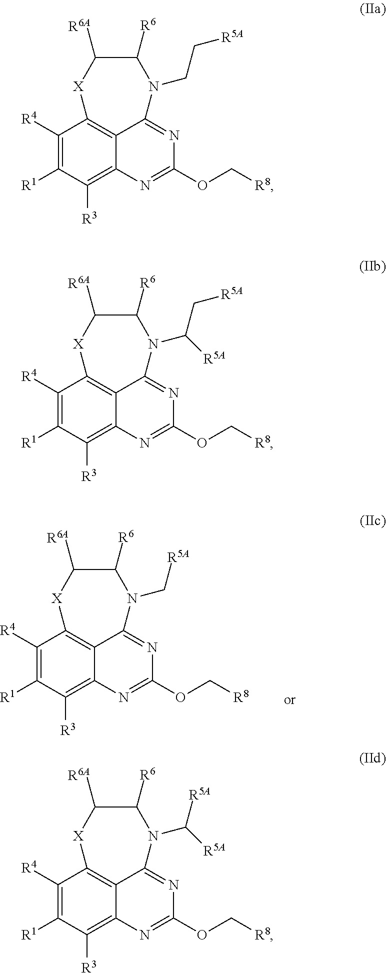

or a stereoisomer, atropisomer, tautomer, or pharmaceutically acceptable salt thereof, where R¹, R³, R⁴, R⁵, R⁶, R^{6A}, R⁸, and X are as described herein.

In one embodiment where R² is O-L¹-R⁸, where R⁸ is R⁹-substituted 4-10 membered heterocycle comprising N, S, or O. In another such embodiment, R⁸ is 4-10 membered heterocycle comprising one N heteroatom. In another such embodiment, R⁸ is 4, 5, 6, or 7 membered monocyclic heterocycle comprising one N heteroatom. In another such embodiment, R⁸ is 5 or 6 membered monocyclic heterocycle comprising one N heteroatom. In another such embodiment, R⁸ is 5 or 6 membered monocyclic heterocycle comprising one O heteroatom. In another such embodiment, R⁸ is a 6, 7, 8, or 9 membered fused bicyclic heterocycle comprising one N heteroatom. In another such embodiment, Re is 7 or 8 membered fused bicyclic heterocycle comprising one N heteroatom. In another such embodiment, R⁸ is 7 or 8 membered fused bicyclic heterocycle comprising one N heteroatom and one O heteroatom. In one embodiment, R⁸ is pyrrolidinyl or tetrahydrofuranyl.

In such embodiments, each R⁹ is independently halogen, oxo, unsubstituted C₁₋₃ alkyl, unsubstituted C₁₋₃ haloalkyl, unsubstituted C₁₋₃ alkoxy, or R¹⁰-substituted or unsubstituted C₁₋₃ alkylidene. In another such embodiment, each R⁹ is independently halogen, oxo, or R¹⁰-substituted or unsubstituted C₁₋₃ alkylidene. In one embodiment, each R⁹ is independently unsubstituted C₁₋₃ alkyl or unsubstituted C₁₋₃ alkoxy. In one embodiment, each R⁹ is R¹⁰-substituted or unsubstituted C₃₋₄ cycloalkyl or R¹⁰-substituted or unsubstituted 3 or 4-membered heterocycle. In one embodiment, two R⁹ together form an R¹⁰-substituted or unsubstituted C₃₋₅ cycloalkyl. In one such embodiment, two R⁹ together form a R¹⁰-substituted cyclopropyl. In one such embodiment, two R⁹ together form a R¹⁰-substituted cyclopropyl where R¹⁰ is halogen (e.g. F or Cl). In one embodiment, where two R⁹ together form a R¹⁰-substituted cyclopropyl, the cyclopropyl is attached at a single carbon of Re. In one embodiment, two R⁹ together form a R¹⁰-substituted cyclopropyl, the cyclopropyl is attached at two separate carbon atoms of R⁸. In another such embodiment, two R⁹ together form a unsubstituted C₃₋₅ heterocycle comprising one or more oxygen atoms. In one such embodiment, the heterocycle is a 1,3-dioxolanyl.

In one embodiment, R¹⁰ is hydrogen or halogen. In one embodiment, R¹⁰ is hydrogen. In another embodiment, R¹⁰ is halogen. In one such embodiment, R¹⁰ is F.

In one embodiment, where R² is O-L¹-Re, Re is

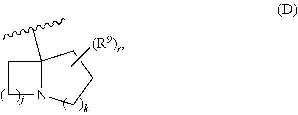

wherein,
R⁹ is halogen, —OCF₃, —OCHF₂, —OCH₂F, R¹⁰-substituted or unsubstituted C₁₋₃ alkylidene, or two R⁹ together form a R¹⁰-substituted or unsubstituted C₃₋₅ cycloalkyl;
r is an integer of 0-12;
j is 1, 2, or 3; and
k is 1 or 2.

In one embodiment, where R² is O-L¹-Re, R⁸ is

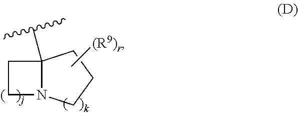

wherein,
R⁹ is halogen or R¹⁰-substituted or unsubstituted C₁₋₃ alkylidene;
r is an integer of 0-12;
j is 1, 2, or 3; and
k is 1 or 2.

In one such embodiment, r is 0, 1, 2, 3, or 4. In another such embodiment, r is 0, 1, 2, or 3. In one embodiment, R⁸ is

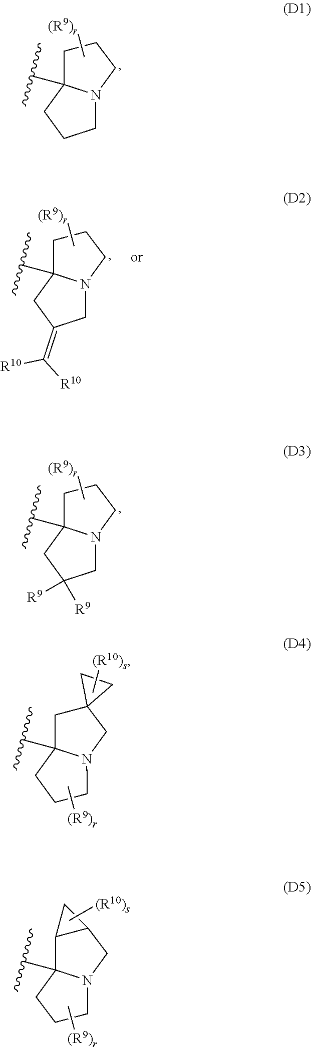

where R⁹, R¹⁰ and r are as described herein and s is 1 or 2.

In one such embodiment, r is 0, 1, 2, 3, or 4. In another such embodiment, r is 0, 1, 2, or 3. In one embodiment, R⁸ is

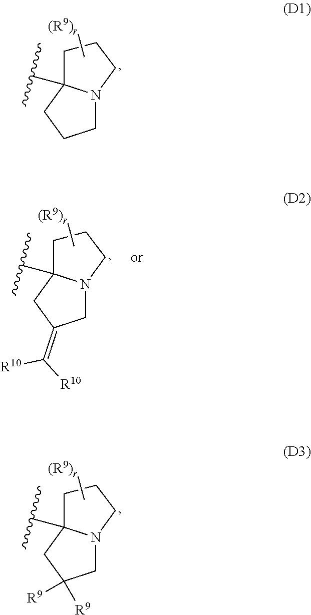

where R⁹, R¹⁰ and r are as described herein.

In one such embodiment, R⁹ is independently halogen or R¹⁰-substituted or unsubstituted C₁₋₃ alkylidene; each R¹⁰ is independently hydrogen or halogen; and r is 1 or 2.

In one embodiment, R⁸ is

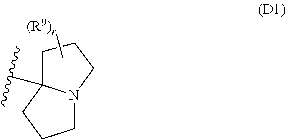

where r is 0.

In another embodiment, R⁸ is

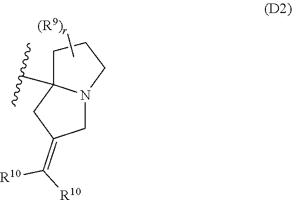

where r is 0 and each R¹⁰ is independently hydrogen or F. In one such embodiment, r is 0 and each R¹⁰ is hydrogen. In another such embodiment, r is 0 and each R¹⁰ is F. In another such embodiment, r is 0 where one R¹⁰ is hydrogen and one R¹⁰ is F. In another such embodiment, each R¹⁰ is independently hydrogen or F, r is 1 or 2, and R⁹ is F.

In another embodiment, R⁸ is

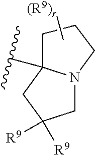

where r is 0 and each R⁹ is independently hydrogen or halogen. In one such embodiment, each R⁹ is F and r is 0. In one such embodiment, each R⁹ is F and r is 1.

In another embodiment where R² is O-L¹-R⁸, R⁸ is

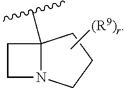

In one such embodiment, r is 1 and R⁹ is halogen, oxo, or unsubstituted C₁ alkylidene. In one such embodiment, two R⁹ together form a R¹⁰-substituted or unsubstituted C₃₋₅ cycloalkyl.

In one embodiment, R⁸ is

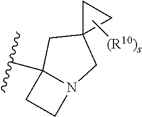

where R¹⁰ is halogen and s is 1 or 2. In one such embodiment, R⁸ is

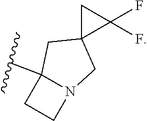 [SMILES: *C12CCN1CC1(C2)CC1(F)F]

In another embodiment where R² is O-L¹-R⁸, R⁸ is

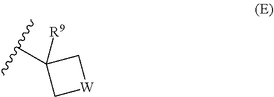

wherein
R⁹ is hydrogen or unsubstituted C₁₋₃ alkyl; and
W is O, SO₂, or NR¹²; and
R¹² is hydrogen, unsubstituted C1-3 alkyl, or unsubstituted C1-3 haloalkyl.

In one such embodiment, W is O and R⁹ is methyl. In another such embodiment, W is NR¹², where R¹² is unsubstituted C₁₋₃ haloalkyl and R⁹ is hydrogen. In another such embodiment, W is SO₂ and R⁹ is hydrogen.

In one embodiment of the compounds or a pharmaceutically acceptable salt thereof described herein, R⁸ is azetidinyl, oxetanyl, or thietanedioxide.

In further embodiments provided herein, R⁸ is a moiety having formula:

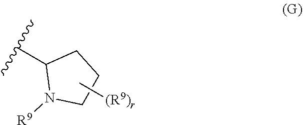

wherein,
R⁹ is independently halogen, oxo, or unsubstituted C₁₋₃ alkyl;
or wherein two R⁹ together form a C₃₋₅ cycloalkyl or 3-5 membered heterocycle; and
r is 1 or 2.

In one such embodiment, R⁸ is a moiety having formula (G) where R⁹ and r are as described herein. In one such embodiment, two R⁹ together form a R¹⁰-substituted or unsubstituted cyclopropyl moiety. In one embodiment, the cyclopropyl moiety is unsubstituted. In another embodiment, the cyclopropyl moiety is substituted with halogen (e.g. F). In one such embodiment, two R⁹ together form a R¹⁰-substituted or unsubstituted cyclopropyl fused to the pyrrolidinyl. In another such embodiment, two R⁹ together form a R¹⁰-substituted or unsubstituted cyclopropyl moiety that is spiro to the pyrrolidinyl. In one embodiment, R⁹ is oxo and r is 1. In another such embodiment, R⁹ is F and r is 1 or 2. In one embodiment, the N—R⁹, R⁹ is C₁₋₃ alkyl. In one such embodiment, R⁹ is methyl.

In another embodiment, R⁸ is a moiety having formula:

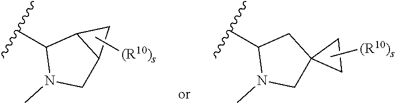

where R¹⁰ is halogen and s is 1 or 2.

In another embodiment, R⁸ is a moiety having formula:

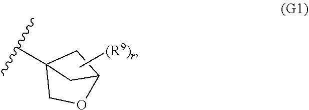

wherein R⁹ and r as described herein.

In another embodiment, R⁸ is a moiety having formula:

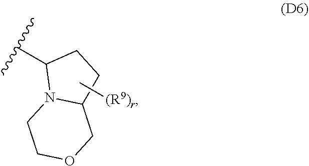

wherein R⁹ and r are as described herein.

In still another embodiment, R⁸ is R⁹-substituted or unsubstituted C₁₋₃ alkyl. In one such embodiment, R⁸ is a moiety of formula:

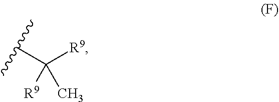

where each R⁹ is independently unsubstituted C₁₋₃ alkyl or unsubstituted C₁₋₃ alkoxy.

In another embodiment, Re is a moiety having formula:

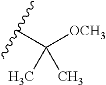 [SMILES: *C(C)(C)OC]

In one embodiment, R⁸ is:

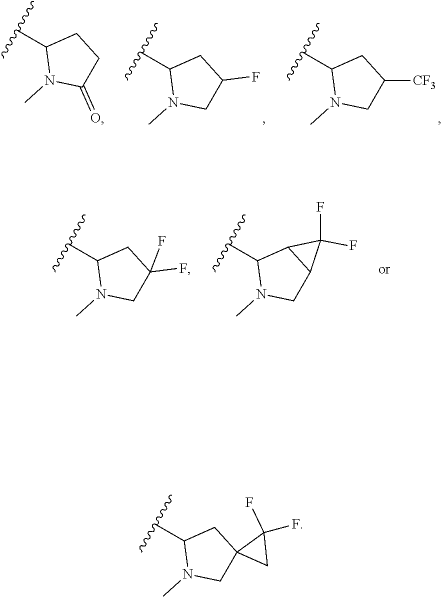

In one embodiment, R⁸ is:

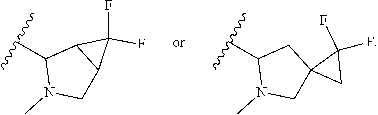

In one embodiment, R⁸ is:

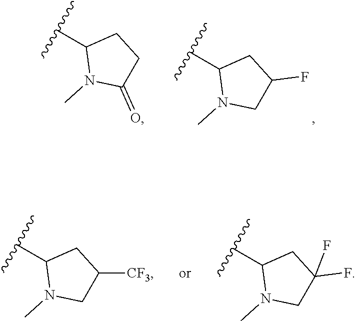

In one embodiment, R⁸ is:

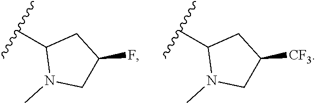

In another embodiment, R⁸ is:

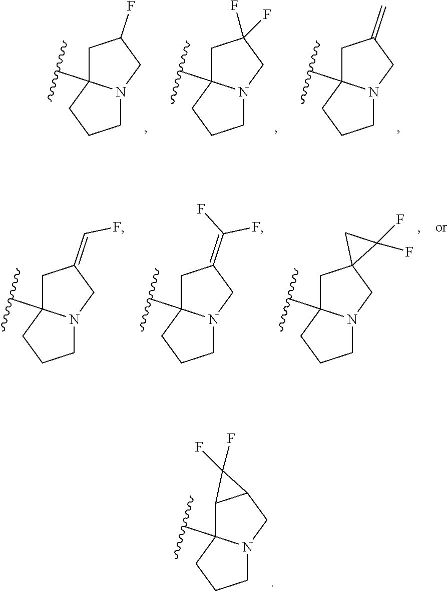

In another embodiment, R⁸ is:

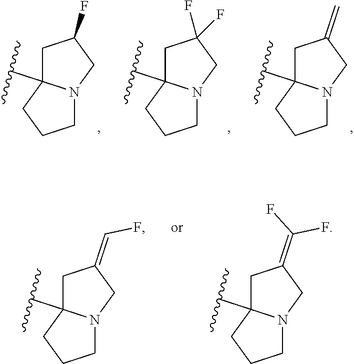

In another embodiment, R⁸ is:

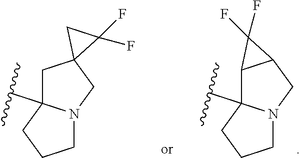 [SMILES: *C12CCCN1CC1(C2)CC1(F)F ; *C12CCCN1CC1C2C1(F)F]

In still another embodiment, R⁸ is:

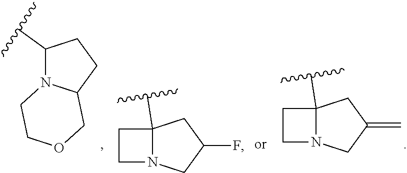

In still another embodiment, R⁸ is:

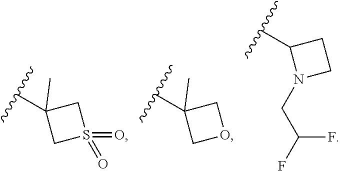

In still another embodiment, R⁸ is:

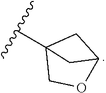 [SMILES: *C12COC(C1)C2]

In still another embodiment, R⁸ is:

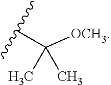 [SMILES: *C(C)(C)OC]

In still another embodiment, R² is:

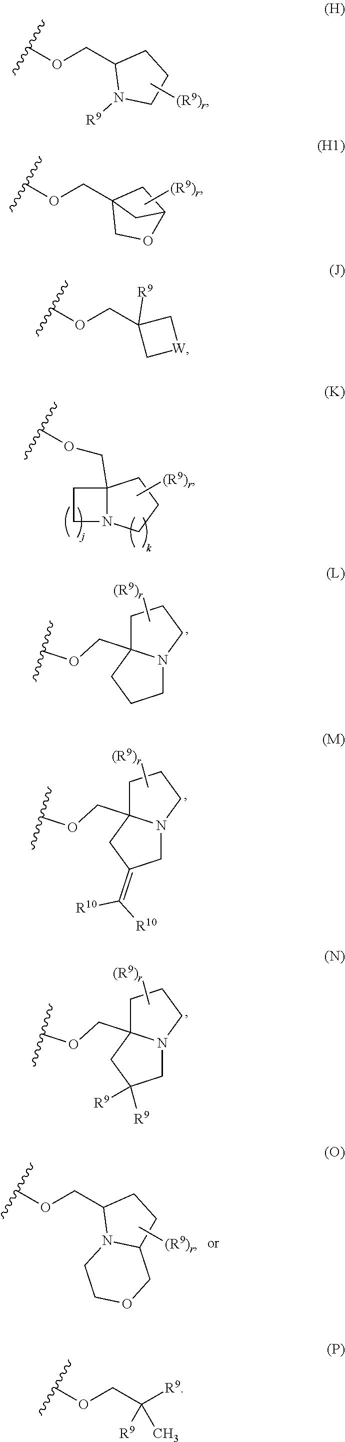

where R⁹, R¹⁰, r, j, and k are as described herein. In one embodiment, R⁹ is halogen or R¹⁰-substituted or unsubstituted C₁₋₃ alkylidene. In another such embodiment, R⁹ is halogen, oxo, R¹⁰-substituted or unsubstituted C₁₋₃ alkylidene, and r is independently 0, 1, or 2.

In one embodiment, R² is:

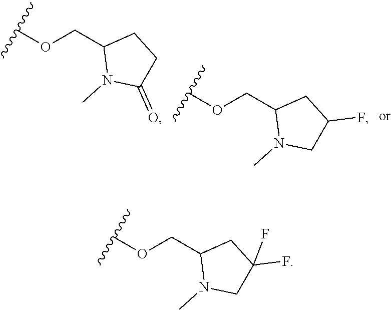

In another embodiment, R² is:

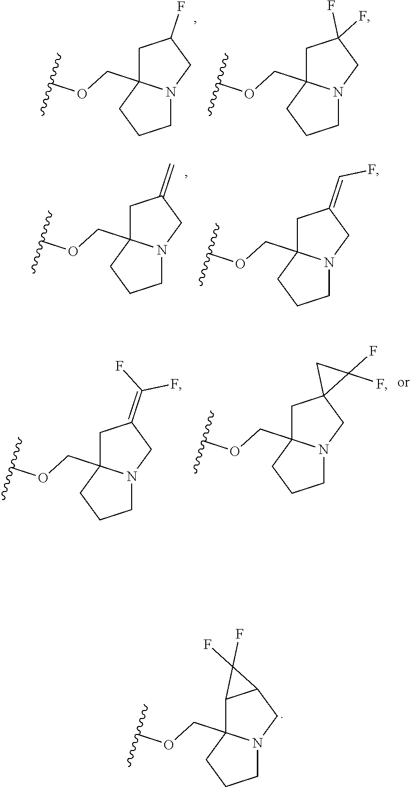

In another embodiment, R² is:

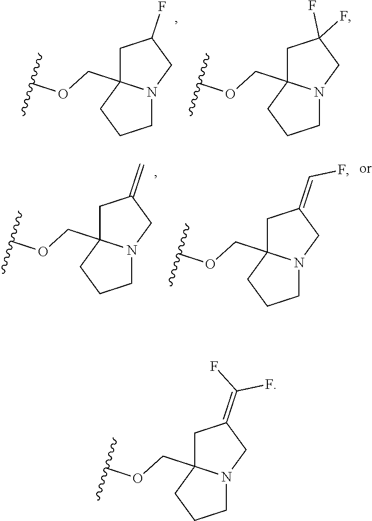

In still another embodiment, R² is:

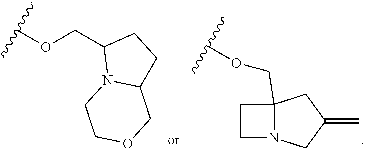 [SMILES: *OCC1CCC2COCCN12 ; *OCC12CCN1CC(=C)C2]

In still another embodiment, R² is:

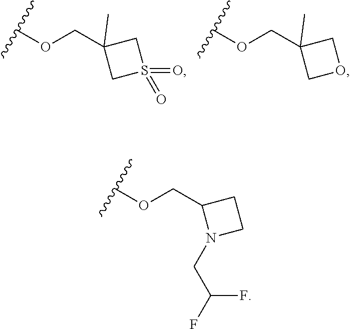

In still another embodiment, R² is:

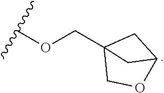 [SMILES: *OCC12COC(C1)C2]

In still another embodiment, R² is:

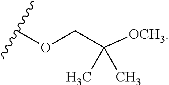 [SMILES: *OCC(C)(C)OC]

In another embodiment, R² is R^{8A}-substituted or unsubstituted C₁₋₃ alkyl or R^{8B}-substituted or unsubstituted 4-10 membered heterocycle. In one embodiment, each R^{8A} is independently R^{9A}-substituted or unsubstituted C₁₋₃ alkyl or R^{9A}-substituted or unsubstituted C₁₋₃ alkoxy. In one embodiment, each R^{8B} is independently R^{8A} is independently R^{9A}-substituted or unsubstituted alkoxy or R^{9A}-substituted or unsubstituted 4-6 membered heterocycle In another embodiment, each R^{9A} is independently R^{9A}-substituted or unsubstituted C₃₋₄ cycloalkyl, or R^{9A}-substituted or unsubstituted 4-6 membered heterocycle. In one embodiment, R^{9A} is R⁹-substituted or unsubstituted 4-10 membered heterocycle comprising N. In another embodiment, R⁹ is independently halogen, unsubstituted C₁₋₃ alkyl, or R¹⁰-substituted or unsubstituted C₁₋₃ alkylidene.

In one embodiment, R² is R^{8A}-substituted or unsubstituted C₁₋₃ alkyl, where R^{8A} is R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle.

In one embodiment, R^{9A} is independently halogen, oxo, unsubstituted C₁₋₃ alkyl, unsubstituted C₁₋₃ haloalkyl, unsubstituted C₁₋₃ alkoxy, or unsubstituted C₁₋₃ alkylidene. In another such embodiment, R^{9A} is independently R^{9A} is independently halogen, oxo, or unsubstituted C₁₋₃ alkylidene. In still another embodiment, R^{9A} is R⁹-substituted or unsubstituted 4-10 membered heterocycle comprising N, S, or O.

In one embodiment, R² is R^{8A}-substituted or unsubstituted C₁₋₃ alkyl, where R^{8A} is R^{9A}-substituted or unsubstituted C₁₋₃ alkyl.

In one embodiment, R² is R^{8A}-substituted or unsubstituted C₁₋₃ alkyl, where R^{8A} is R^{9A}-substituted or unsubstituted C₁₋₃ alkoxy. In one such embodiment, R^{9A} is independently R⁹-substituted or unsubstituted C₃₋₄ cycloalkyl, or R⁹-substituted or unsubstituted 4-10 membered heterocycle comprising one N heterocycle. In another such embodiment, R^{9A} is independently R⁹-substituted or unsubstituted 5 or 6 membered monocyclic heterocycle comprising one N heterocycle or 7 or 8 membered fused bicyclic heterocycle comprising one N heterocycle. In such embodiments, R⁹ is independently halogen, oxo, unsubstituted C₁₋₃ alkyl, or R¹⁰-substituted or unsubstituted C₁₋₃ alkylidene, where R¹⁰ is as described herein.

In another embodiment, R² is R^{8A}-substituted or unsubstituted C₁₋₃ alkyl, where R^{8A} is R^{9A}-substituted or unsubstituted C₃₋₄ cycloalkyl. In one embodiment, each R^{8B} is independently halogen, oxo, unsubstituted C₁₋₃ alkyl, unsubstituted C₁₋₃ haloalkyl, unsubstituted C₁₋₃ alkoxy, or unsubstituted C₁₋₃ alkylidene.

In one embodiment, R² is R^{8B}-substituted or unsubstituted 4-10 membered heterocycle. In one such embodiment, R^{8B} is halogen, oxo, or unsubstituted C₁₋₃ alkylidene. In one embodiment, R² is R^{8B}-substituted or unsubstituted 4, 5, or 7 membered heterocycle comprising one N heteroatom.

In one embodiment, R³ and R⁴ are each independently hydrogen, —CN, halogen, or unsubstituted C₁₋₃ alkyl. In one embodiment, R³ and R⁴ are each independently hydrogen, unsubstituted C₁₋₃ alkyl, or unsubstituted cyclopropyl. In one embodiment, R³ and R⁴ are each independently hydrogen, halogen, or unsubstituted C₁₋₃ alkyl. In one embodiment, R³ and R⁴ are each independently hydrogen or halogen. In one embodiment, both R³ and R⁴ are not hydrogen. In another embodiment, one of R³ and R⁴ is hydrogen and the other is halogen. In one such embodiment, R³ is hydrogen and R⁴ is halogen. In one embodiment, R³ is halogen. In one such embodiment, R³ is F or Cl. In another embodiment, R⁴ is hydrogen. In another embodiment, R⁴ is halogen. In one such embodiment, R⁴ is F or Cl.

In one embodiment, R⁵ is R^{5A}-substituted or unsubstituted C₁₋₄ alkyl, R^{5A}-substituted or unsubstituted C₁₋₆ haloalkyl, R^{5A}-substituted or unsubstituted C₃₋₁₀ cycloalkyl, R^{5A}-substituted or unsubstituted 3-10 membered heterocycle, or R^{5A}-substituted or unsubstituted 5-10 membered heteroaryl.

Where R⁵ is R^{5A}-substituted or unsubstituted C₃₋₁₀ cycloalkyl, the cycloalkyl can be a monocycle such as, for example, cyclopropyl, cyclobutyl, cyclopentyl, or cyclohexyl. Where R⁵ is R^{5A}-substituted or unsubstituted C₃₋₁₀ cycloalkyl, the cycloalkyl can be a bicycle such as, for example, 3,5-, 3-6, 4,5-, 4-6, 5,5-, or 5,6- where one or both of the fused rings of the bicyclic moiety comprises a R^{5A}-substituted or unsubstituted cycloalkyl moiety.

Where R⁵ is R^{5A}-substituted or unsubstituted 3-10 membered heterocycle, the heterocycle can be a monocycle such as, for example, aziridinyl, oxiranyl, or thiranyl. Where R⁵ is R^{5A}-substituted or unsubstituted 3-10 membered heterocycle, the heterocycle can be a monocycle such as, for example, azetidinyl, oxetanyl, or thietanyl. Where R⁵ is R^{5A}-substituted or unsubstituted 3-10 membered heterocycle, the heterocycle can be a monocycle such as, for example, pyrrolidinyl, tetrahydrofuranyl, thiophenyl, imidazolidinyl, oxathiolidinyl, thiazolidinyl, piperidinyl, oxanyl, thianyl, or morpholino. Where R⁵ is R^{5A}-substituted or unsubstituted 3-10 membered heterocycle, the heterocycle can be a bicycle such as, for example, 3,5-, 3-6, 4,5-, 4-6, 5,5-, or 5,6- where one or both of the fused rings of the bicyclic moiety comprises a R^{5A}-substituted or unsubstituted heterocycle moiety.

Where R⁵ is or R^{5A}-substituted or unsubstituted 5-10 membered heteroaryl, the heteroaryl can be a monocycle such as, for example, pyrrolyl, imidazolyl, furanyl, thiophenyl, triazolyl, tetrazolyl, pyridinyl, pyranyl, triazinyl, pyrazolyl, pyrazinyl, pyridonyl, pyrimidinyl, or pyridazinyl. Where R⁵ is R^{5A}-substituted or unsubstituted 3-10 membered heterocycle, the heterocycle can be a bicycle such as, for example, 3,5-, 3-6, 4,5-, 4-6, 5,5-, or 5,6- where one or both of the fused rings of the bicyclic moiety comprises a R^{5A}-substituted or unsubstituted heteroaryl moiety. In one such embodiment, R⁵ is pyrrolopyridinyl, or pyrazolopyridinyl.

In another embodiment, R⁵ is R^{5A}-substituted or unsubstituted C₁₋₆ alkyl or R^{5A}-substituted or unsubstituted C₁₋₆ haloalkyl. In another embodiment, R⁵ is R^{5A}-substituted or unsubstituted C₃₋₁₀ cycloalkyl, R^{5A}-substituted or unsubstituted 3-10 membered heterocycle, or R^{5A}-substituted or unsubstituted 5-10 membered heteroaryl.

In one embodiment, R⁵ is R^{5A}-substituted or unsubstituted C₁₋₆ alkyl. In one such embodiment, R⁵ is R^{5A}-substituted or unsubstituted C₁₋₃ alkyl. In one embodiment, R⁵ is R^{5A}-substituted C₁₋₃ alkyl where R^{5A} is as described herein. Where R⁵ is R^{5A}-substituted C₁₋₃ alkyl, R⁵ may be a moiety of formula:

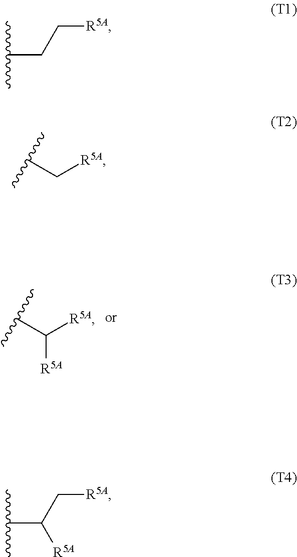

where R^{5A} is as described herein. Where R⁵ is R^{5A}-substituted C₁₋₃ alkyl, R⁵ may be a moiety of formula (T1), (T2), (T3), or (T4), where R^{5A} is halogen, CF3, CHF2, CH2F, CN, OR¹¹, SR¹², SO₂R¹², NR¹³R¹⁴, C(O)N(R¹¹)₂, C(O)R¹¹, R^{5B}-substituted or unsubstituted C₁₋₆ alkyl, R^{5B}-substituted or unsubstituted C₃₋₆ cycloalkyl, R^{5B}-substituted or unsubstituted 3-6 membered heterocycle, or R^{5B}-substituted or unsubstituted 5-9 membered heteroaryl. In one such embodiment, at least one R^{5A} is R^{5B}-substituted or unsubstituted 3-6 membered heterocycle, or R^{5B}-substituted or unsubstituted 5-9 membered heteroaryl. In another such embodiment, two R^{5A} together form R^{5B}-substituted or unsubstituted cyclopropyl.

Where R⁵ is R^{5A}-substituted C₁₋₃ alkyl, R⁵ may be a moiety of formula:

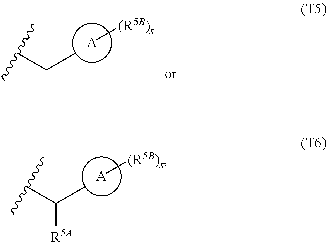

wherein,
R^{5A} and R^{5B} are as described herein;
Ring A is a 3-6 membered heterocycle or 5-9 membered heteroaryl comprising at least one N heteroatom; and
s is 0, 1, 2, or 3.

Where R⁵ is R^{5A}-substituted C₁₋₃ alkyl, R⁵ may be a moiety of formula (T5) or (T6), where R^{5B} is halogen, oxo, CN, OH, OCH₃, NR¹³R¹⁴, SR¹², R^{5C}-substituted or unsubstituted C₁₋₃ alkyl, R^{5C}-substituted or unsubstituted C₁₋₃ haloalkyl, R^{5C}-substituted or unsubstituted 3-6 membered heterocycle, or R^{5C}-substituted or unsubstituted 5-6 membered heteroaryl. In another embodiment, R^{5B} is halogen, oxo, CN, OH, OCH₃, NR¹³R¹⁴, SR¹², or R^{5C}-substituted or unsubstituted C₁₋₃ alkyl. In another embodiment, R^{5B} is oxo, CN, OH, NR¹³R¹⁴, SR¹², or R^{5C}-substituted or unsubstituted C₁₋₃ alkyl. Where R^{5B} is R^{5C}-substituted or unsubstituted C₁₋₃ alkyl, in such embodiments, R^{5C} is halogen, CN, C(O)CH₃, OH, OCH₃, CF₃, CHF₂, CH₂F, NR¹³R¹⁴, SCH₃, SO₂NH₂, SO₂CH₃, or unsubstituted C₁₋₃ alkyl. In one particular embodiment, R⁵ is NR¹³R¹⁴, where R13 and R14 are as described herein. In one such embodiment, at least one of R13 and R14 is hydrogen. In another such embodiment, at least one of R13 and R15 is R¹⁵-substituted or unsubstituted C₁₋₆ alkyl, R¹⁵-substituted or unsubstituted C₃₋₆ cycloalkyl, or R¹⁵-substituted or unsubstituted 3-6 membered heterocycle.

In one embodiment, where R⁵ is R^{5A}-substituted C₁₋₃ alkyl, R⁵ may be a moiety of formula (T5) or (T6), where R^{5B} is NR¹³R¹⁴, and NR¹³R¹⁴ is NH2. In another embodiment, where R⁵ is R^{5A}-substituted C₁₋₃ alkyl, R⁵ may be a moiety of formula (T5) or (T6), where R^{5B} is NR¹³R¹⁴, and NR¹³R¹⁴ is NHR¹⁴ where R14 is R¹⁵-substituted or unsubstituted C₁₋₆ alkyl, R¹⁵-substituted or unsubstituted C₃₋₆ cycloalkyl, or R¹⁵-substituted or unsubstituted 3-6 membered heterocycle.

In one embodiment, each R^{5A} is independently halogen, oxo, CN, OR¹¹, SR¹², SO₂R¹², NR¹³R¹⁴, C(O)N(R¹¹)₂, or C(O)R¹¹. In one embodiment, each R^{5A} is independently R^{5B}-substituted or unsubstituted C₁₋₆ alkyl, R^{5B}-substituted or unsubstituted C₁₋₆ haloalkyl. In one embodiment, each R^{5A} is independently R^{5B}-substituted or unsubstituted C₃₋₆ cycloalkyl, R^{5B}-substituted or unsubstituted 3-6 membered heterocycle, R^{5B}-substituted or unsubstituted phenyl, or R^{5B}-substituted or unsubstituted 5-9 membered heteroaryl.

In one embodiment, each R^{5A} is OR¹¹, where R¹¹ is hydrogen, methyl, ethyl, CH₂F, CHF₂, CF₃, cyclopropyl, cyclopropylmethyl, oxetanyl, or oxetanylmethyl. In one embodiment, each R^{5A} is independently halogen, oxo, CN, OH, OCH₃, SH, SO₂NH₂, NH₂, NH(CH₃), N(CH₃)₂, N(CH₃)(CH₂CH₃), C(O)NH₂, or C(O)CH₃.

Where each R^{5A} is independently R^{5B}-substituted or unsubstituted 5-9 membered heteroaryl, the heteroaryl moiety can be a 5, 6, or 7-membered monocyclic heteroaryl. In one such embodiment, the heteroaryl moiety is a 5, 6, or 7-membered moiety comprising at least one N heteroatom. In another such embodiment, the heteroaryl moiety is a 5, 6, or 7-membered moiety comprising at least one O heteroatom. In still another embodiment, the heteroaryl moiety is a 5, 6, or 7-membered moiety comprising an S heteroatom.

Where each R^{5A} is independently R^{5B}-substituted or unsubstituted 5-9 membered heteroaryl, the heteroaryl moiety can be a 7, 8, or 9-membered bicyclic heteroaryl. In one such embodiment, the heteroaryl moiety is a 7, 8, or 9-membered moiety comprising at least one N heteroatom. In another such embodiment, the heteroaryl moiety is a 7, 8, or 9-membered moiety comprising at least one O heteroatom. In still another embodiment, the heteroaryl moiety is a 7, 8, or 9-membered moiety comprising an S heteroatom.

In one embodiment, each R^{5B} is independently halogen, oxo, CN, OH, OCH₃, NR¹³R¹⁴, SR¹², SO₂R¹², C(O)N(R¹¹)₂, or C(O)R¹¹. In one embodiment, each R^{5B} is independently R^{5C}-substituted or unsubstituted C₁₋₃ alkyl. In one embodiment, each R^{5B} is independently R^{5C}-substituted or unsubstituted C₁₋₃ haloalkyl. In one embodiment, each R^{5B} is independently R^{5C}-substituted or unsubstituted C₃₋₆ cycloalkyl. In one such embodiment, each R^{5B} is independently cyclopropyl or cyclobutyl. In one embodiment, each R^{5B} is independently R^{5C}-substituted or unsubstituted 3-6 membered heterocycle. In one such embodiment, each R^{5B} is independently a 4, 5, or 6 membered heterocycle. In another such embodiment, the 4, 5, or 6 membered heterocycle comprises at least one N heteroatom. In another such embodiment, the 4, 5, or 6 membered heterocycle comprises at least one O heteroatom. In one embodiment, each R^{5B} is independently R^{5C}-substituted or unsubstituted phenyl. In one embodiment, each R^{5B} is independently or R^{5C}-substituted or unsubstituted 5-6 membered heteroaryl.

In one embodiment, R^{5C} is independently halogen, oxo, CN, C(O)CH₃, OH, OCH₃, CF₃, CHF₂, CH₂F, NR¹³R¹⁴, SCH₃, SO₂NH₂, or SO₂CH₃. In one embodiment, R^{5B} is R^{5C}-substituted C₁₋₃ alkyl, where R^{5C} is independently halogen, oxo, CN, C(O)CH₃, OH, OCH₃, CF₃, CHF₂, CH₂F, NR¹³R¹⁴, SCH₃, SO₂NH₂, or SO₂CH₃. In another embodiment, R⁵¹ is R^{5C}-substituted C₁₋₃ alkyl, where R^{5C} is independently halogen, oxo, CN, C(O)CH₃, OH, OCH₃, CF₃, CHF₂, CH₂F, NR¹³R¹⁴, SCH₃, SO₂NH₂, SO₂CH₃ or unsubstituted C₁₋₃ alkyl. In another such embodiment, R^{5C} is independently unsubstituted C₁₋₃ alkyl. In one embodiment, R^{5C} is independently unsubstituted C₃₋₄ cycloalkyl or unsubstituted 3-4 membered heterocycle.

In one embodiment, R¹¹ is hydrogen or unsubstituted C₁₋₃ alkyl. R¹¹ may be hydroxy. R¹¹ may be methyl. R¹¹ may be ethyl.

In one embodiment, R¹² is NH₂, NHCH₃, or N(CH₃)₂, or unsubstituted C₁₋₃ alkyl. R¹² may be NH₂ or unsubstituted C₁₋₃ alkyl. In one such embodiment, R¹² is NH₂. In another such embodiment, R¹² is methyl.

In one embodiment, R¹³ and R¹⁴ are independently hydrogen, C(O)R¹¹, R¹⁵-substituted or unsubstituted C₁₋₆ alkyl, R¹⁵-substituted or unsubstituted C₃₋₆ cycloalkyl, R¹⁵-substituted or unsubstituted 3-6 membered heterocycle, or R¹⁵-substituted or unsubstituted 3-6 membered heteroaryl.

In one embodiment, each R¹³ and R¹⁴ are independently hydrogen, C(O)R¹¹, or R¹⁵-substituted or unsubstituted C₁₋₆ alkyl. In one embodiment, each R¹³ and R¹⁴ are independently R¹⁵-substituted or unsubstituted C₃₋₆ cycloalkyl or R¹⁵-substituted or unsubstituted 3-6 membered heterocycle. R¹³ and R¹⁴ may each independently be hydrogen or R¹⁵-substituted or unsubstituted C₁₋₆ alkyl. In another embodiment, may each independently be hydrogen or R¹⁵-substituted or unsubstituted C₁₋₃ alkyl. In one embodiment, one of R¹³ and R¹⁴ is hydrogen. In another embodiment, one of R¹³ and R¹⁴ is R¹⁵-substituted or unsubstituted C₁₋₆ alkyl.

In one embodiment, R¹⁵ is halogen, CN, C(O)CH₃, OH, OCH₃, CF₃, CHF₂, CH₂F, NH₂, NHCH₃, N(CH₃)₂, SO₂NH₂, or SO₂CH₃. In one embodiment, R¹⁵ is CN, C(O)CH₃, OH, OCH₃, CF₃, CHF₂, CH₂F, NH₂, NHCH₃, N(CH₃)₂, SO₂NH₂, or SO₂CH₃. In another embodiment, R¹⁵ is R¹⁶-substituted or unsubstituted C₁₋₃ alkyl. In still another embodiment, R¹⁵ is R¹⁶-substituted or unsubstituted C₃₋₆ cycloalkyl, R¹⁶-substituted or unsubstituted 3-6 membered heterocycle, R¹⁶-substituted or unsubstituted 5-9 membered aryl, or R¹⁶-substituted or unsubstituted 5-9 membered heteroaryl. In one embodiment, R¹⁵ is R¹⁶-substituted C₁₋₃ alkyl, where each R16 is independently

In one embodiment, each R¹⁶ is halogen, CN, C(O)CH₃, OH, OCH₃, CF₃, CHF₂, CH₂F, NH₂, NHCH₃, N(CH₃)₂, SO₂NH₂, SO₂CH₃, unsubstituted C₁₋₃ alkyl, unsubstituted C₃₋₆ cycloalkyl, unsubstituted 3-6 membered heterocycle, unsubstituted 5-9 membered aryl, or unsubstituted 5-9 membered heteroaryl.

In one embodiment, each R¹⁶ is independently halogen, CN, C(O)CH₃, OH, OCH₃, CF₃, CHF₂, CH₂F, NH₂, NHCH₃, N(CH₃)₂, SO₂NH₂, SO₂CH₃. In one embodiment, each R¹⁰ is independently R¹⁷-substituted or unsubstituted C₁₋₃ alkyl. In one embodiment, each R¹⁶ is independently R¹⁷-substituted or unsubstituted C₃₋₆ cycloalkyl. In one embodiment, each R¹⁶ is independently R¹⁷-substituted or unsubstituted 3-6 membered heterocycle. In one embodiment, each R¹⁶ is independently R¹⁷-substituted or unsubstituted 4, 5, or 6 membered heterocycle. In one such embodiment, the 4, 5, or 6 membered heterocycle comprises at least one N heteroatom. In one embodiment, each R¹⁶ is independently R¹⁷-substituted or unsubstituted phenyl. In one embodiment, each R¹⁶ is independently R¹⁷-substituted or unsubstituted 5-9 membered heteroaryl. In one embodiment, each R¹⁶ is independently R¹⁷-substituted or unsubstituted 4, 5, or 6 membered heteroaryl. In one such embodiment, the 4, 5, or 6 membered heteroaryl comprises at least one N or O heteroatom. In another such embodiment, the 4, 5, or 6 membered heteroaryl comprises at least one N heteroatom. In another such embodiment, the 4, 5, or 6 membered heteroaryl comprises at least one O heteroatom.

In one embodiment, each R¹⁷ is independently halogen, CN, C(O)CH₃, OH, OCH₃, CF₃, CHF₂, CH₂F, NH₂, NHCH₃, N(CH₃)₂, SO₂NH₂, SO₂CH₃. In another embodiment, each R¹⁷ is independently CN, NH₂, NHCH₃, N(CH₃)₂, SO₂NH₂, SO₂CH₃, or unsubstituted C₁₋₃ alkyl. In one such embodiment, each R¹⁷ is independently CN, NH₂, NHCH₃, N(CH₃)₂, SO₂NH₂, SO₂CH₃, or methyl.

In one embodiment, Ring A is a 3-6 membered heterocycle. In one such embodiment, Ring A is a 4, 5, or 6 membered ring comprising one or more N heteroatoms. In another embodiment, Ring is a 5-9 membered heteroaryl comprising at least one N heteroatom. In one such embodiment, Ring A is 6 membered heteroaryl comprising at least one N heteroatom. In one embodiment, Ring A is azetidinyl, thietanyl 1,1-dioxide, imidazolyl, thiazolyl, isothiazolyl, triazolyl, pyrazolyl, pyrazinyl, pyridonyl, pyridinyl, pyrimidinyl, pyridazinyl, pyrrolopyridinyl, or pyrazolopyridinyl. In another embodiment, Ring A is imidazolyl, isothiazolyl, or triazolyl. In another embodiment, A is pyrazolyl, pyridonyl, pyridinyl, pyrimidinyl, or pyridazinyl.

In one embodiment, where R⁵ is R^{5A}-substituted C₁₋₃ alkyl, R⁵ may be a moiety of formula (T5) or (T6), where the moiety comprises a moiety of formula

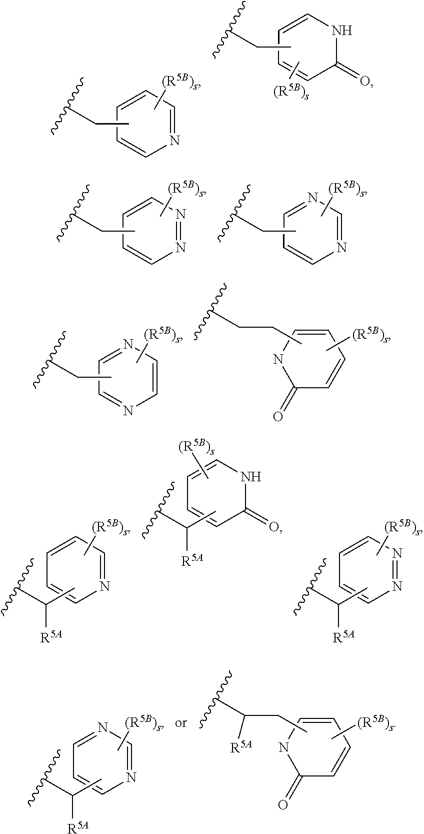

In one embodiment, where R⁵ is R^{5A}-substituted C₁₋₃ alkyl, R⁵ may be a moiety of formula (T5) or (T6), where the moiety comprises a moiety of formula

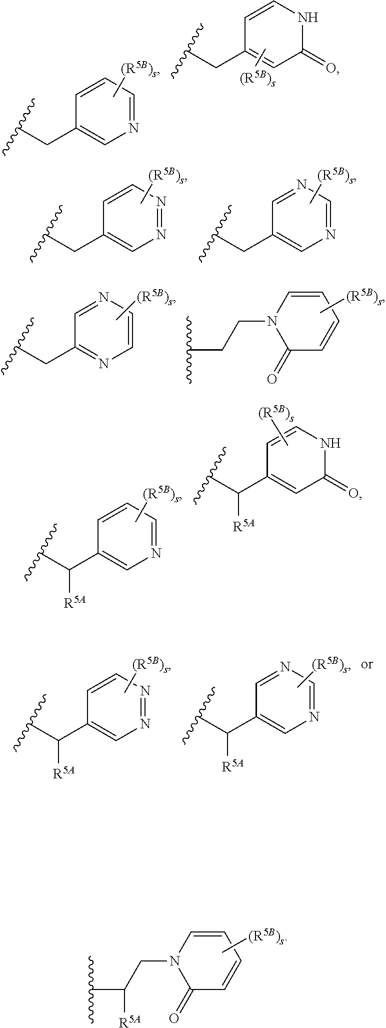

In one embodiment, where R⁵ is R^{5A}-substituted C₁₋₃ alkyl, R⁵ may be a moiety of (T1), where R^{5A} is as described herein. Where R⁵ is a moiety of (T1), in one embodiment, R^{5A} is CN, OH, C(O)N(R¹¹)₂, C(O)R¹¹, SO₂R¹², NR¹³R¹⁴, R^{5B}-substituted or unsubstituted azetidinyl, or R^{5B}-substituted or unsubstituted oxetanyl. Where R⁵ is a moiety of (T1), in one embodiment, R^{5A} is NR¹³R¹⁴, where R¹³ and R¹⁴ are independently hydrogen, R¹⁵-substituted or unsubstituted C₁₋₆ alkyl, R¹⁵-substituted or unsubstituted C₃₋₆ cycloalkyl, or R¹⁵-substituted or unsubstituted 3-6 membered heterocycle. In one embodiment, one of R¹³ and R¹⁴ is hydrogen. In another embodiment, at least one of R¹³ and R¹⁴ is R¹⁵-substituted or unsubstituted C₁₋₆ alkyl. In one such embodiment, at least one of R¹³ and R¹⁴ is methyl. In another embodiment, at least one of R¹³ and R¹⁴ is R¹⁵-substituted or unsubstituted C₁₋₃ alkyl. Where at least one of R¹³ and R¹⁴ is R¹⁵-substituted or unsubstituted C₁₋₃ alkyl, R¹⁵ can be C(O)CH₃, OH, OCH₃, CF₃, CHF₂, CH₂F, NH₂, NHCH₃, N(CH₃)₂, R¹⁶-substituted or unsubstituted C₁₋₃ alkyl, R¹⁶-substituted or unsubstituted C₃₋₆ cycloalkyl, R¹⁶-substituted or unsubstituted 3-6 membered heterocycle, R¹⁶-substituted or unsubstituted 5-9 membered aryl, or R¹⁶-substituted or unsubstituted 5-9 membered heteroaryl.

In one embodiment of the compounds or a pharmaceutically acceptable salt thereof described herein, R⁵ is R^{5A}-substituted or unsubstituted C₃₋₁₀ cycloalkyl. In one such embodiment, R⁵ is R^{5A}-substituted C4-6 monocyclic cycloalkyl. In another such embodiment, R⁵ is R^{5A}-substituted C7-10 bicyclic cycloalkyl where at least one of the rings is a cycloalkyl moiety. In one embodiment, a C3-5 cycloalkyl is bound spiro to the carbon of another ring.

In one embodiment, R⁵ is R^{5A}-substituted or unsubstituted 3-10 membered heterocycle. In one such embodiment, R⁵ is R^{5A}-substituted 3-7 membered monocyclic heterocycle. In another such embodiment, R⁵ is R^{5A}-substituted 7-10 membered bicyclic heterocycle where at least one of the rings is a heterocycle moiety. In one embodiment, a 3-5 membered heterocycle is bound spiro to the carbon of another ring.

In one embodiment, R⁵ is R^{5A}-substituted or unsubstituted 5-10 membered heteroaryl. In one such embodiment, R⁵ is R^{5A}-substituted 5 or 6 membered monocyclic heteroaryl. In another such embodiment, R⁵ is R^{5A}-substituted 7-10 membered bicyclic heteroaryl where at least one of the rings is a heteroaryl moiety.

In one embodiment, R⁵ is R^{5A}-substituted or unsubstituted cyclopentapyridinyl, R^{5A}-substituted or unsubstituted pyrrolopyridinyl, pyrazolopyridinyl, or imidazopyridinyl.

In one embodiment, R⁶ and R^{6A} are independently hydrogen or Re-substituted or unsubstituted C₁₋₆ alkyl. In another embodiment, R⁶ and R^{6A} are independently hydrogen, NR¹³R¹⁴, or Re-substituted or unsubstituted C₁₋₆ alkyl. In still another embodiment, R⁶ and R^{6A} are independently hydrogen, halogen, or Re-substituted or unsubstituted C₁₋₆ alkyl. In one embodiment, R⁶ is R^{6B}-substituted or unsubstituted C₁₋₃ alkyl. In one embodiment, R⁶ is R^{6B}-substituted C₁₋₃ alkyl. In one embodiment, R^{6A} is R^{6B}-substituted or unsubstituted C₁₋₃ alkyl. In one embodiment, R^{6A} is R^{6B}-substituted C₁₋₃ alkyl. In one embodiment, at least one of R⁶ and R^{6A} is independently hydrogen. In one embodiment, R⁶ is hydrogen. In another embodiment, at least one of R⁶ and R^{6A} is independently R^{6B}-substituted or unsubstituted C₁₋₃ alkyl, where Re is halogen, CN, or OH. In one such embodiment, one of R⁶ and R^{6A} is hydrogen and the other is R^{6B}-substituted or unsubstituted C₁₋₃ alkyl. In one such embodiment, R^{6B} is halogen, CN, or OH. In one embodiment, R⁶ is methyl, CH2CN, or CH2OH and R^{6A} is hydrogen. In one embodiment, R^{6A} is methyl, CH2CN, or CH2OH and R⁶ is hydrogen.

In one embodiment, R^{6B} is halogen, CN, OH, or OCH₃. In one embodiment, R^{6B} is CF₃, CHF₂, or CH₂F. In one embodiment, R^{6B} is or unsubstituted C₁₋₃ alkyl. In one embodiment, R^{6B} is CN.

In one such embodiment, R¹ is as described herein. In another such embodiment, R¹ is a moiety of formula (A1), (A2), or (B). In another such embodiment, R² is a moiety of formula (H), (J), (K), (L), (M), (N), (O), or (P).

In another such embodiment, the compound is a compound of formula (11) having formula;

In one embodiment, the compound of formula (I) has formula:

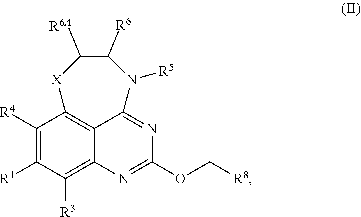

or a stereoisomer, atropisomer, tautomer, or pharmaceutically acceptable salt thereof, where R¹ is a moiety of formula (A1), (A2), or (B); R⁸ is a moiety of formula (D1), (D2), (D3), (E), (G), or (G1); and X is O. In another embodiment of the compound of formula (II), R¹ is a moiety of formula (A1) or (A2); R⁸ is a moiety of formula (D1), (D2), (D3), (E), (G), or (G1); and X is O. In still another embodiment, R¹ is a moiety of formula (B); R⁸ is a moiety of formula (D1), (D2), (D3), (E), (G), or (G1); and X is O. In some such embodiments, R⁵ is a moiety of (T1), (T2), (T3), (T4), (T5), or (T6).

In one embodiment, the compound of formula (I) has formula:

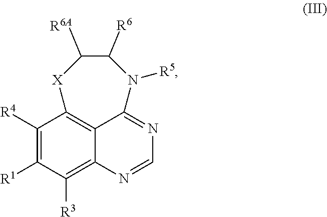

or a stereoisomer, atropisomer, tautomer, or pharmaceutically acceptable salt thereof, where R¹ is a moiety of formula (A1), (A2), or (B); and X is O. In another embodiment of the compound of formula (III), R¹ is a moiety of formula (A1) or (A2); and X is O. In still another embodiment, R¹ is a moiety of formula (B); and X is O. In some such embodiments, R⁵ is a moiety of (T1), (T2), (T3), (T4), (T5), or (T6).

In one embodiment, R^{e} of the compounds described herein is:

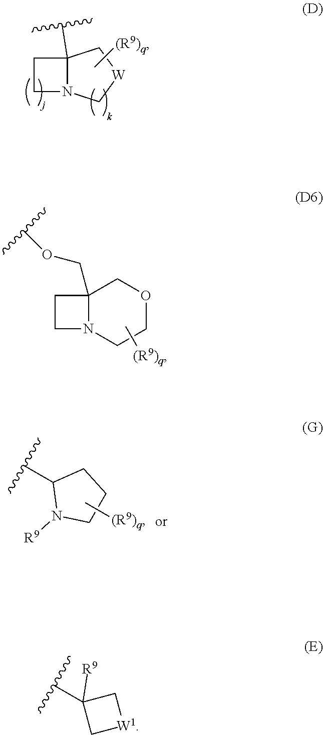

In one embodiment, R⁸ of the compounds described herein is:

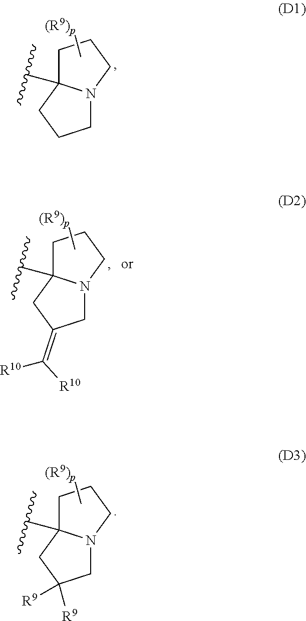

In another embodiment, R⁸ of the compounds described herein is:

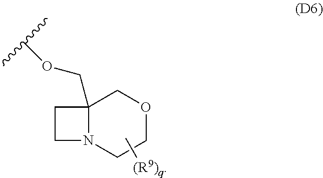

In one embodiment, R⁸ of the compounds described herein is:

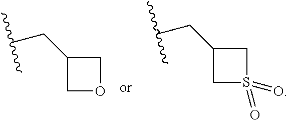 [SMILES: *CC1COC1 ; *CC1CS(=O)(=O)C1]

Further provided herein are compounds of formula:

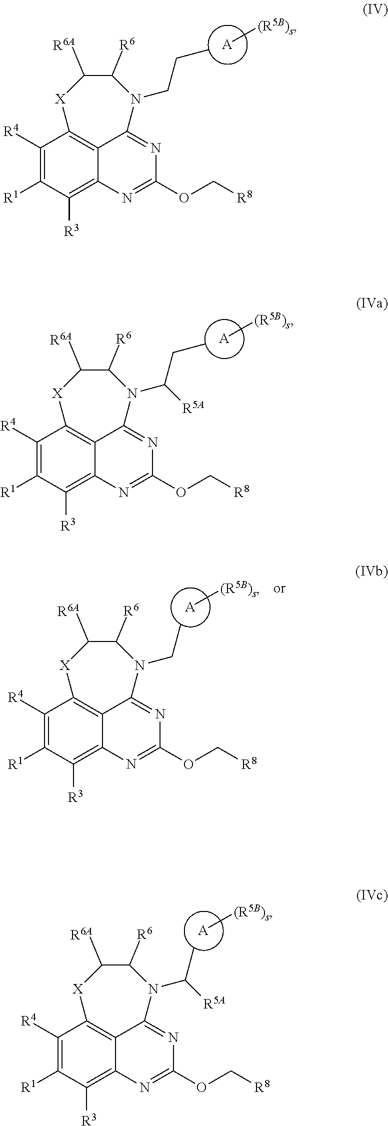

or a stereoisomer, atropisomer, tautomer, or pharmaceutically acceptable salt thereof wherein R¹, R³, R⁴, R⁵, R^{5A}, R^{5B}, R⁶, R^{6A}, R⁸, X, and Ring A are as defined herein. In one embodiment of the compounds of formula (II), (IIa), (IIb), (IIc), (IId), (IV), (IVa), (IVb), or (IVc), R⁸ is:

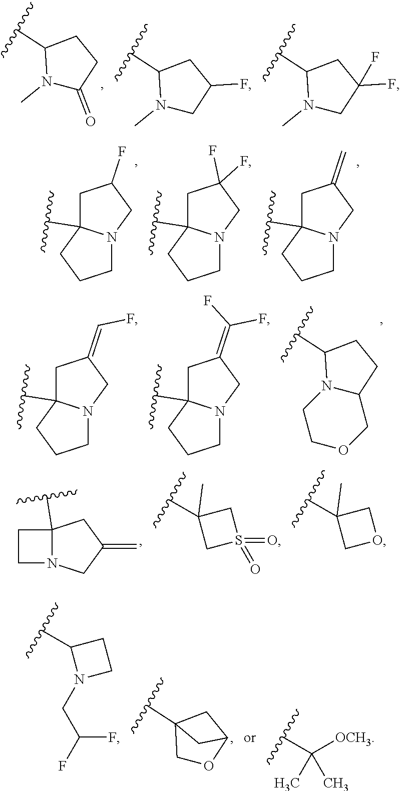

In another embodiment of the compounds of formula (I), (II), (IIa), (IIb), (IIc), (IId), (IV), (IVa), (IVb), or (IVc) R⁸ is:

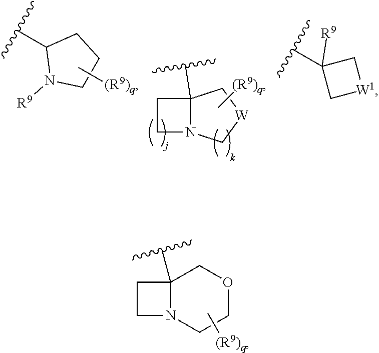

where R9, W, W1, q, j, and k are as described herein.

In another embodiment of the compounds of formula (I), (II), (IIa), (IIb), (IIc), (IId), (IV), (IVa), (IVb), or (IVc) R⁸ is:

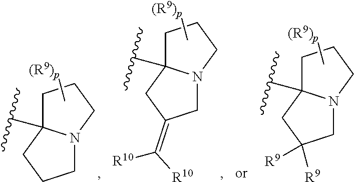

where R9 and R10 are as described herein.

In another embodiment of the compounds of formula (I), (II), (IIa), (IIb), (IIc), (IId), (IV), (IVa), (IVb), or (IVc) R⁸ is:

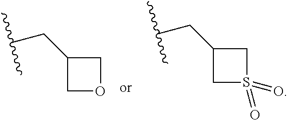 [SMILES: *CC1COC1 ; *CC1CS(=O)(=O)C1]

Further provided herein are compounds of formula:

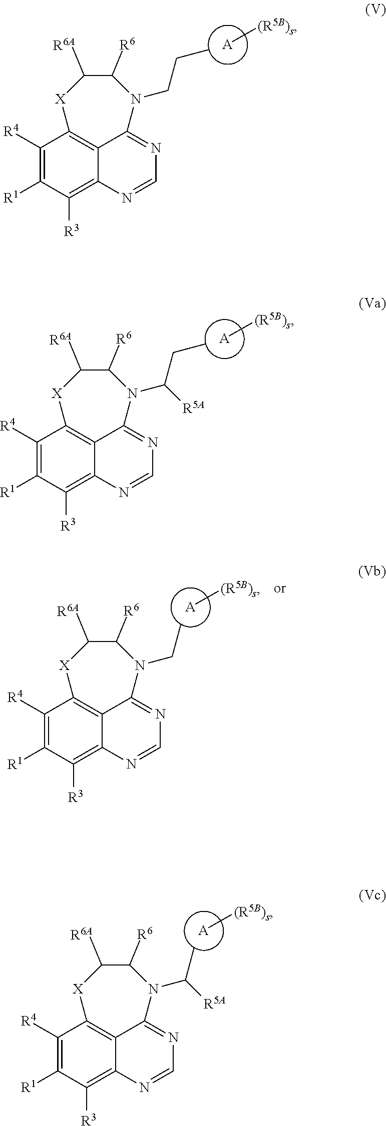

or a stereoisomer, atropisomer, tautomer, or pharmaceutically acceptable salt thereof, wherein R1, R³, R⁴, R⁵, R^{5A}, R^{5B}, R⁶, R^{6A}, R⁸, X, and Ring A are as defined herein.

In one embodiment, the compound of formula (I), (II), or (III) or a stereoisomer, atropisomer, tautomer, or pharmaceutically acceptable salt thereof is a compound of Table 1.

TABLE 1

| Ex. | |
|---|---|
| No. | Structure |
| 1 | 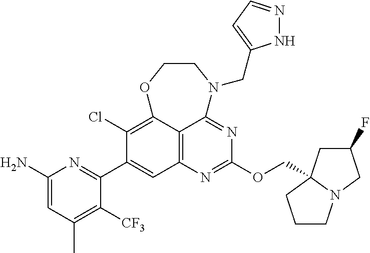 [SMILES: Cc1cc(N)nc(-c2cc3nc(OC[C@@]45CCCN4C[C@H](F)C5)nc4c3c(c2Cl)OCCN4Cc2ccn[nH]2)c1C(F)(F)F] |
| 2 | 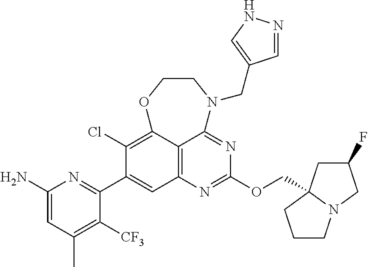 [SMILES: Cc1cc(N)nc(-c2cc3nc(OC[C@@]45CCCN4C[C@H](F)C5)nc4c3c(c2Cl)OCCN4Cc2cn[nH]c2)c1C(F)(F)F] |
| 3 | 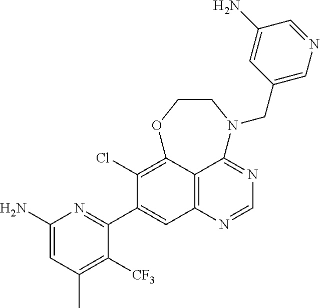 [SMILES: Cc1cc(N)nc(-c2cc3ncnc4c3c(c2Cl)OCCN4Cc2cncc(N)c2)c1C(F)(F)F] |
| 4 | 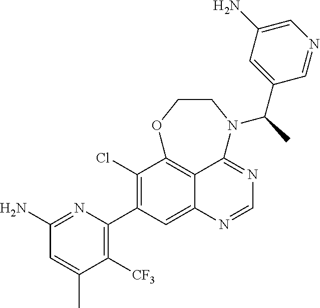 [SMILES: Cc1cc(N)nc(-c2cc3ncnc4c3c(c2Cl)OCCN4[C@H](C)c2cncc(N)c2)c1C(F)(F)F] |
| 5 | 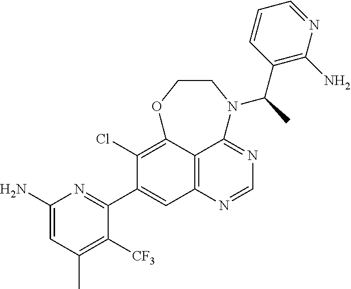 [SMILES: Cc1cc(N)nc(-c2cc3ncnc4c3c(c2Cl)OCCN4[C@H](C)c2cccnc2N)c1C(F)(F)F] |
| 6 | 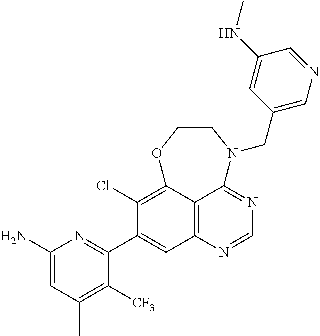 [SMILES: CNc1cncc(CN2CCOc3c(Cl)c(-c4nc(N)cc(C)c4C(F)(F)F)cc4ncnc2c34)c1] |
| 7 | 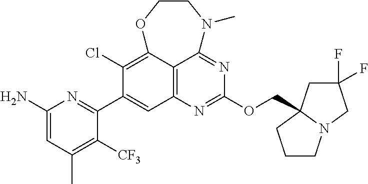 [SMILES: Cc1cc(N)nc(-c2cc3nc(OC[C@]45CCCN4CC(F)(F)C5)nc4c3c(c2Cl)OCCN4C)c1C(F)(F)F] |
| 8 | 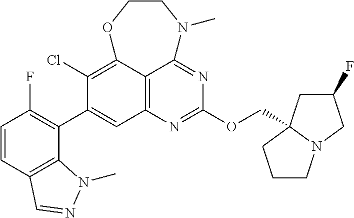 [SMILES: CN1CCOc2c(Cl)c(-c3c(F)ccc4cnn(C)c34)cc3nc(OC[C@@]45CCCN4C[C@H](F)C5)nc1c23] |
| 9 | 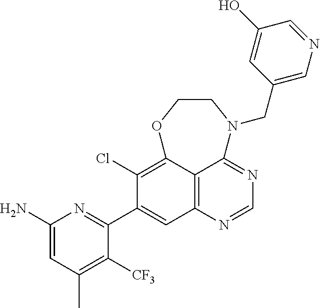 [SMILES: Cc1cc(N)nc(-c2cc3ncnc4c3c(c2Cl)OCCN4Cc2cncc(O)c2)c1C(F)(F)F] |
| 10 | 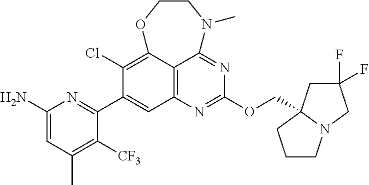 [SMILES: Cc1cc(N)nc(-c2cc3nc(OC[C@@]45CCCN4CC(F)(F)C5)nc4c3c(c2Cl)OCCN4C)c1C(F)(F)F] |
| 11 | 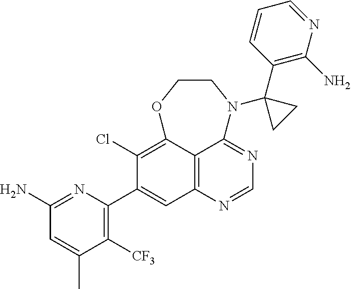 [SMILES: Cc1cc(N)nc(-c2cc3ncnc4c3c(c2Cl)OCCN4C2(c3cccnc3N)CC2)c1C(F)(F)F] |
| 12 | 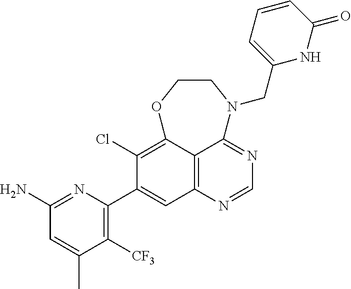 [SMILES: Cc1cc(N)nc(-c2cc3ncnc4c3c(c2Cl)OCCN4Cc2cccc(=O)[nH]2)c1C(F)(F)F] |
| 13 | 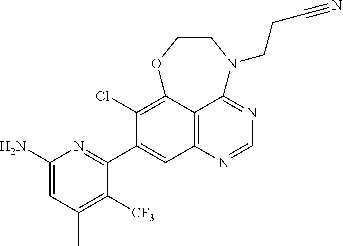 [SMILES: Cc1cc(N)nc(-c2cc3ncnc4c3c(c2Cl)OCCN4CCC#N)c1C(F)(F)F] |
| 14 | 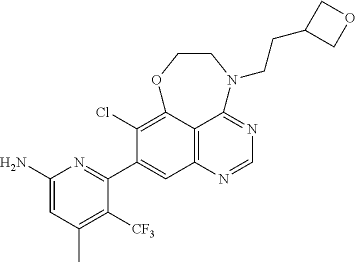 [SMILES: Cc1cc(N)nc(-c2cc3ncnc4c3c(c2Cl)OCCN4CCC2COC2)c1C(F)(F)F] |
| 15 | 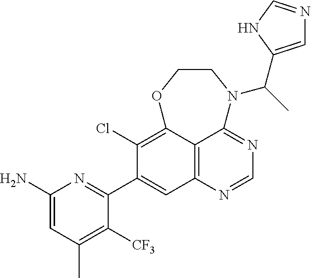 [SMILES: Cc1cc(N)nc(-c2cc3ncnc4c3c(c2Cl)OCCN4C(C)c2cnc[nH]2)c1C(F)(F)F] |
| 16 | 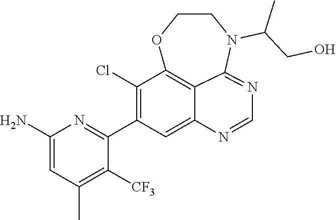 [SMILES: Cc1cc(N)nc(-c2cc3ncnc4c3c(c2Cl)OCCN4C(C)CO)c1C(F)(F)F] |
| 17 | 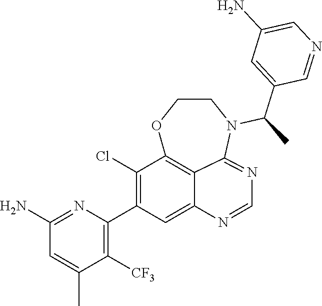 [SMILES: Cc1cc(N)nc(-c2cc3ncnc4c3c(c2Cl)OCCN4[C@H](C)c2cncc(N)c2)c1C(F)(F)F] |
| 18 | 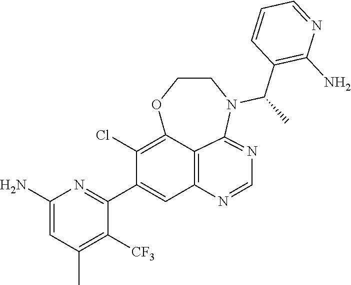 [SMILES: Cc1cc(N)nc(-c2cc3ncnc4c3c(c2Cl)OCCN4[C@@H](C)c2cccnc2N)c1C(F)(F)F] |
| 19 | 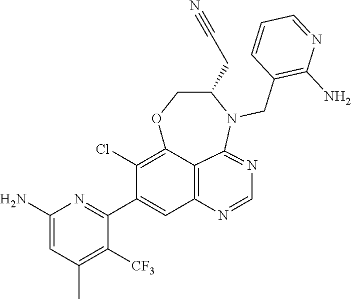 [SMILES: Cc1cc(N)nc(-c2cc3ncnc4c3c(c2Cl)OC[C@H](CC#N)N4Cc2cccnc2N)c1C(F)(F)F] |
| 20 | 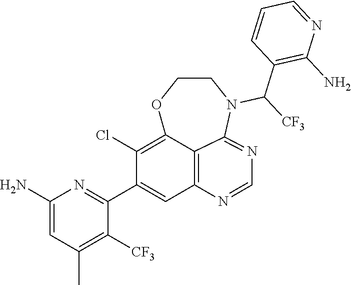 [SMILES: Cc1cc(N)nc(-c2cc3ncnc4c3c(c2Cl)OCCN4C(c2cccnc2N)C(F)(F)F)c1C(F)(F)F] |
| 21 | 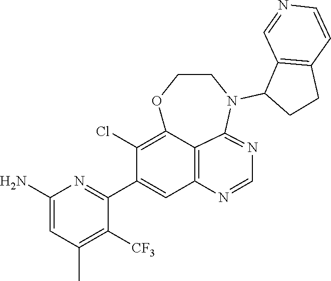 [SMILES: Cc1cc(N)nc(-c2cc3ncnc4c3c(c2Cl)OCCN4C2CCc3ccncc32)c1C(F)(F)F] |
| 22 | 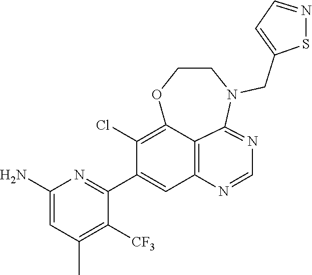 [SMILES: Cc1cc(N)nc(-c2cc3ncnc4c3c(c2Cl)OCCN4Cc2ccns2)c1C(F)(F)F] |
| 23 | 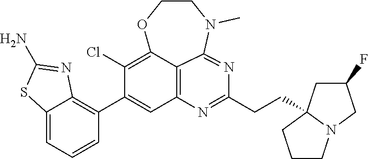 [SMILES: CN1CCOc2c(Cl)c(-c3cccc4sc(N)nc34)cc3nc(CC[C@@]45CCCN4C[C@H](F)C5)nc1c23] |
| 24 | 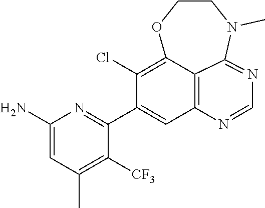 [SMILES: Cc1cc(N)nc(-c2cc3ncnc4c3c(c2Cl)OCCN4C)c1C(F)(F)F] |
| 25 | 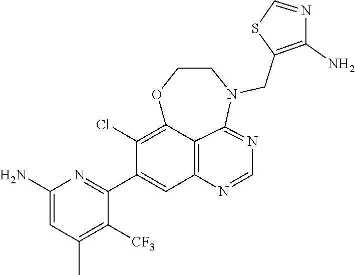 [SMILES: Cc1cc(N)nc(-c2cc3ncnc4c3c(c2Cl)OCCN4Cc2scnc2N)c1C(F)(F)F] |
| 26 | 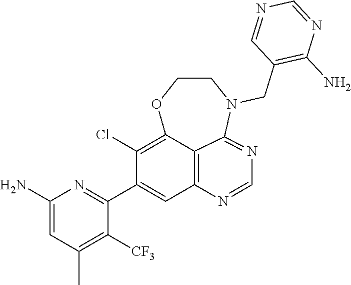 [SMILES: Cc1cc(N)nc(-c2cc3ncnc4c3c(c2Cl)OCCN4Cc2cncnc2N)c1C(F)(F)F] |
| 27 | 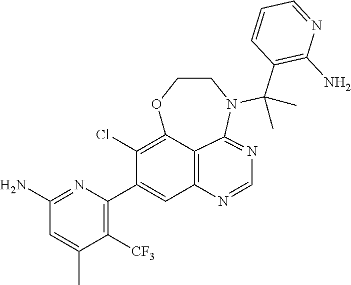 [SMILES: Cc1cc(N)nc(-c2cc3ncnc4c3c(c2Cl)OCCN4C(C)(C)c2cccnc2N)c1C(F)(F)F] |
| 28 | 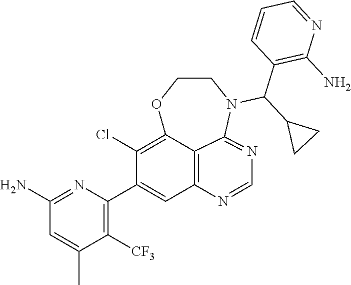 [SMILES: Cc1cc(N)nc(-c2cc3ncnc4c3c(c2Cl)OCCN4C(c2cccnc2N)C2CC2)c1C(F)(F)F] |
| 29 | 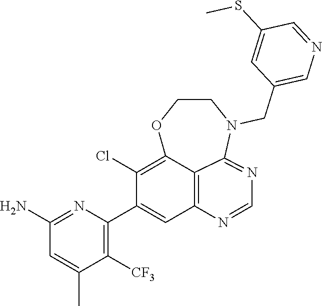 [SMILES: CSc1cncc(CN2CCOc3c(Cl)c(-c4nc(N)cc(C)c4C(F)(F)F)cc4ncnc2c34)c1] |
| 30 | 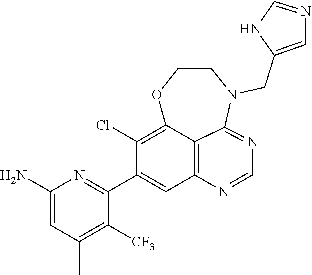 [SMILES: Cc1cc(N)nc(-c2cc3ncnc4c3c(c2Cl)OCCN4Cc2cnc[nH]2)c1C(F)(F)F] |
| 31 | 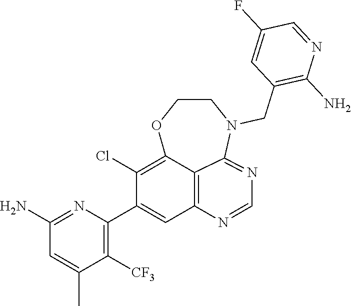 [SMILES: Cc1cc(N)nc(-c2cc3ncnc4c3c(c2Cl)OCCN4Cc2cc(F)cnc2N)c1C(F)(F)F] |
| 32 | 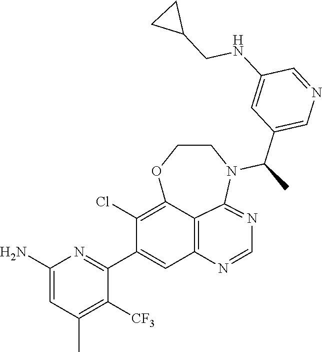 [SMILES: Cc1cc(N)nc(-c2cc3ncnc4c3c(c2Cl)OCCN4[C@H](C)c2cncc(NCC3CC3)c2)c1C(F)(F)F] |
| 33 | 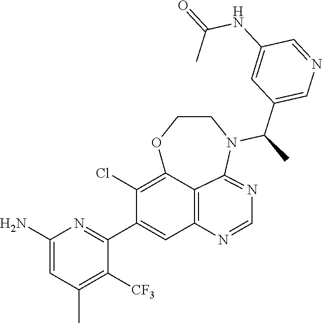 [SMILES: CC(=O)Nc1cncc([C@@H](C)N2CCOc3c(Cl)c(-c4nc(N)cc(C)c4C(F)(F)F)cc4ncnc2c34)c1] |
| 34 | 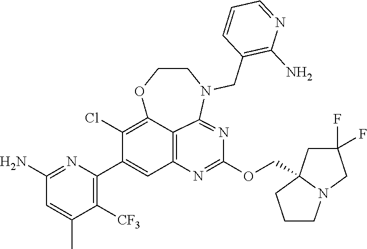 [SMILES: Cc1cc(N)nc(-c2cc3nc(OC[C@@]45CCCN4CC(F)(F)C5)nc4c3c(c2Cl)OCCN4Cc2cccnc2N)c1C(F)(F)F] |
| 35 | 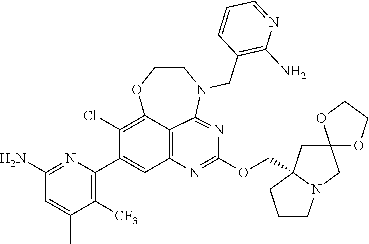 [SMILES: Cc1cc(N)nc(-c2cc3nc(OC[C@@]45CCCN4CC4(C5)OCCO4)nc4c3c(c2Cl)OCCN4Cc2cccnc2N)c1C(F)(F)F] |
| 36 | 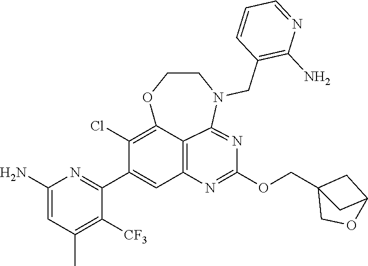 [SMILES: Cc1cc(N)nc(-c2cc3nc(OCC45COC(C4)C5)nc4c3c(c2Cl)OCCN4Cc2cccnc2N)c1C(F)(F)F] |
| 37 | 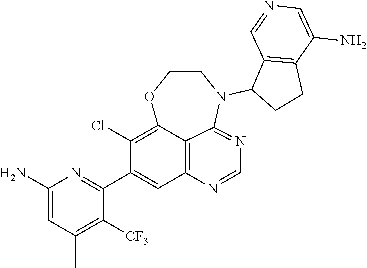 [SMILES: Cc1cc(N)nc(-c2cc3ncnc4c3c(c2Cl)OCCN4C2CCc3c(N)cncc32)c1C(F)(F)F] |
| 38 | 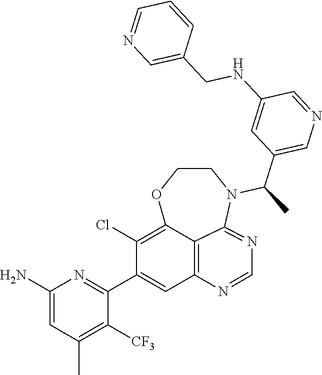 [SMILES: Cc1cc(N)nc(-c2cc3ncnc4c3c(c2Cl)OCCN4[C@H](C)c2cncc(NCc3cccnc3)c2)c1C(F)(F)F] |
| 39 | 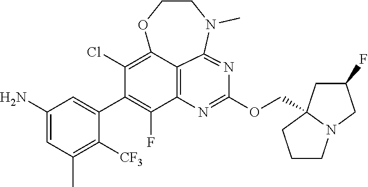 [SMILES: Cc1cc(N)cc(-c2c(Cl)c3c4c(nc(OC[C@@]56CCCN5C[C@H](F)C6)nc4c2F)N(C)CCO3)c1C(F)(F)F] |
| 40 40a 40b | 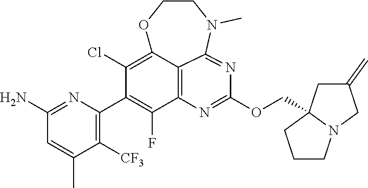 [SMILES: C=C1CN2CCC[C@@]2(COc2nc3c4c(c(Cl)c(-c5nc(N)cc(C)c5C(F)(F)F)c(F)c4n2)OCCN3C)C1] |
| | 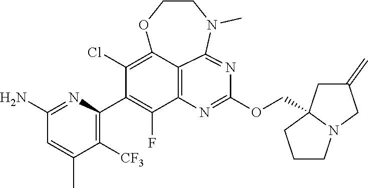 |
| | 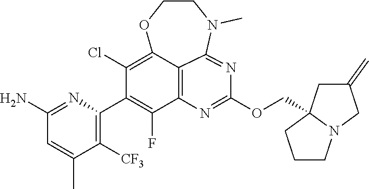 |
| 41 41a 41b | 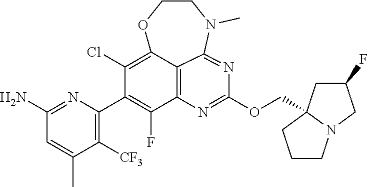 [SMILES: Cc1cc(N)nc(-c2c(Cl)c3c4c(nc(OC[C@@]56CCCN5C[C@H](F)C6)nc4c2F)N(C)CCO3)c1C(F)(F)F] |
| | 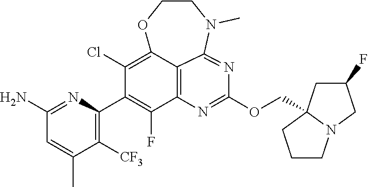 |
| | 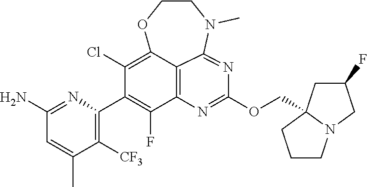 |
| 42 42a 42b | 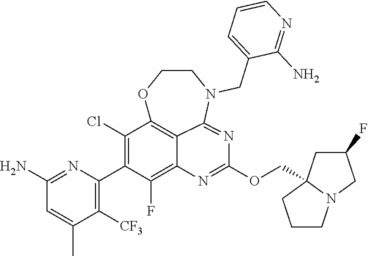 [SMILES: Cc1cc(N)nc(-c2c(Cl)c3c4c(nc(OC[C@@]56CCCN5C[C@H](F)C6)nc4c2F)N(Cc2cccnc2N)CCO3)c1C(F)(F)F] |
| | 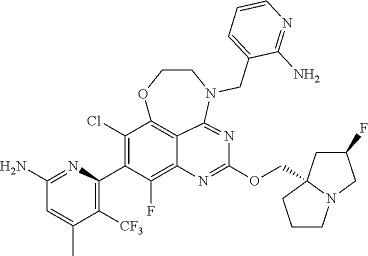 |
| | 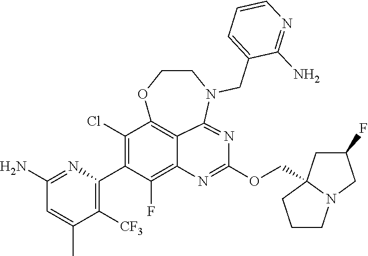 |
| 43 | 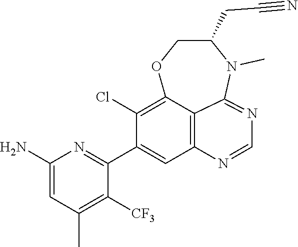 [SMILES: Cc1cc(N)nc(-c2cc3ncnc4c3c(c2Cl)OC[C@H](CC#N)N4C)c1C(F)(F)F] |
| 44 | 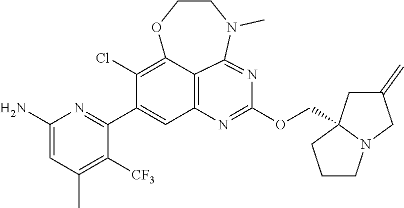 [SMILES: C=C1CN2CCC[C@@]2(COc2nc3c4c(c(Cl)c(-c5nc(N)cc(C)c5C(F)(F)F)cc4n2)OCCN3C)C1] |
| 45 | 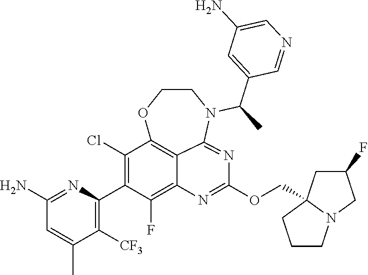 |
| 46 | 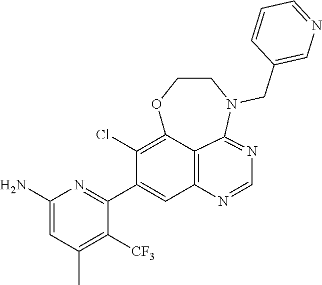 [SMILES: Cc1cc(N)nc(-c2cc3ncnc4c3c(c2Cl)OCCN4Cc2cccnc2)c1C(F)(F)F] |
| 47 47a 47b | 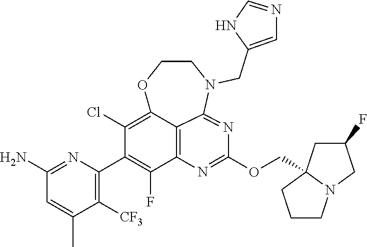 [SMILES: Cc1cc(N)nc(-c2c(Cl)c3c4c(nc(OC[C@@]56CCCN5C[C@H](F)C6)nc4c2F)N(Cc2cnc[nH]2)CCO3)c1C(F)(F)F] |
| | 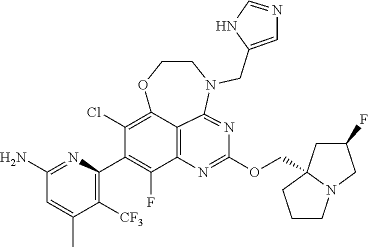 |
| | 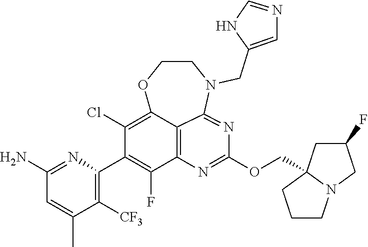 |
| 48 | 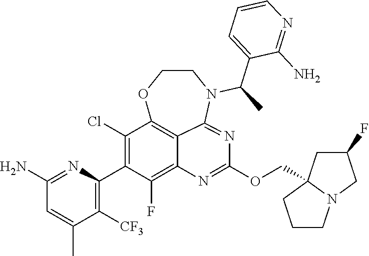 |
| 49 | 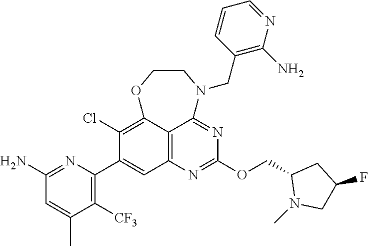 [SMILES: Cc1cc(N)nc(-c2cc3nc(OC[C@@H]4C[C@@H](F)CN4C)nc4c3c(c2Cl)OCCN4Cc2cccnc2N)c1C(F)(F)F] |
| 50 | 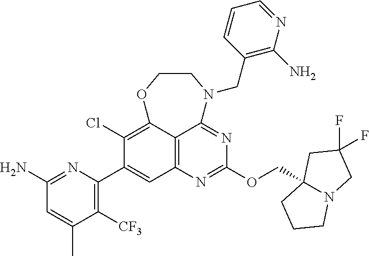 [SMILES: Cc1cc(N)nc(-c2cc3nc(OC[C@@]45CCCN4CC(F)(F)C5)nc4c3c(c2Cl)OCCN4Cc2cccnc2N)c1C(F)(F)F] |
| 51 | 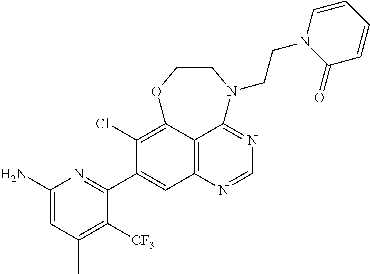 [SMILES: Cc1cc(N)nc(-c2cc3ncnc4c3c(c2Cl)OCCN4CCn2ccccc2=O)c1C(F)(F)F] |
| 52 | 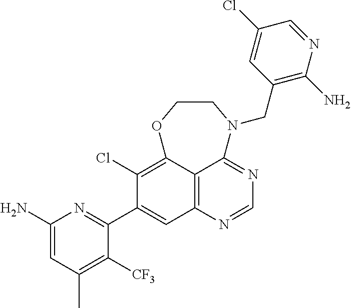 [SMILES: Cc1cc(N)nc(-c2cc3ncnc4c3c(c2Cl)OCCN4Cc2cc(Cl)cnc2N)c1C(F)(F)F] |
| 53 53a 53b | 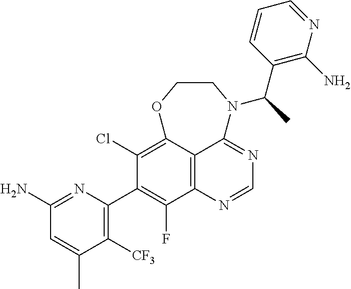 [SMILES: Cc1cc(N)nc(-c2c(Cl)c3c4c(ncnc4c2F)N([C@H](C)c2cccnc2N)CCO3)c1C(F)(F)F] |
| | 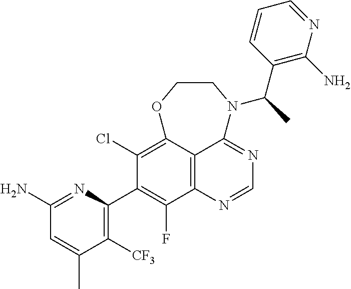 |
| | 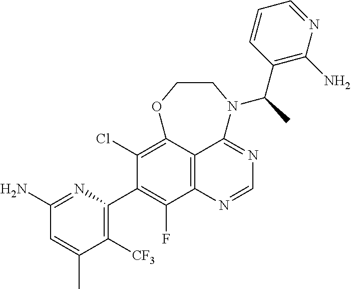 |
| 54 | 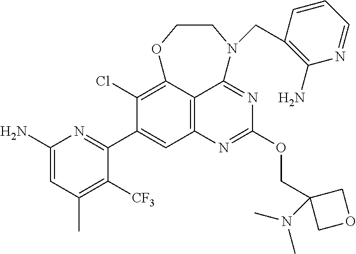 [SMILES: Cc1cc(N)nc(-c2cc3nc(OCC4(N(C)C)COC4)nc4c3c(c2Cl)OCCN4Cc2cccnc2N)c1C(F)(F)F] |
| 55 | 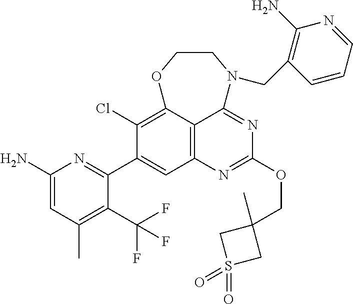 [SMILES: Cc1cc(N)nc(-c2cc3nc(OCC4(C)CS(=O)(=O)C4)nc4c3c(c2Cl)OCCN4Cc2cccnc2N)c1C(F)(F)F] |
| 56 | 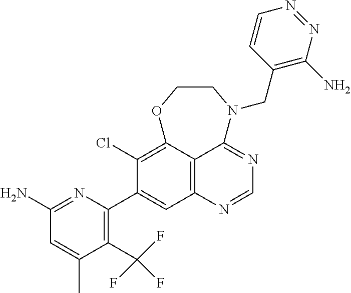 [SMILES: Cc1cc(N)nc(-c2cc3ncnc4c3c(c2Cl)OCCN4Cc2ccnnc2N)c1C(F)(F)F] |
| 57 | 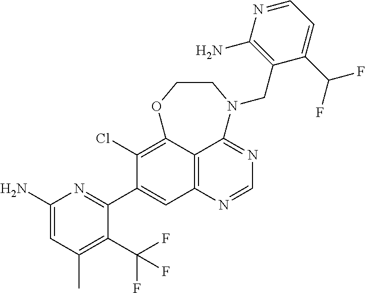 [SMILES: Cc1cc(N)nc(-c2cc3ncnc4c3c(c2Cl)OCCN4Cc2c(C(F)F)ccnc2N)c1C(F)(F)F] |
| 58 | 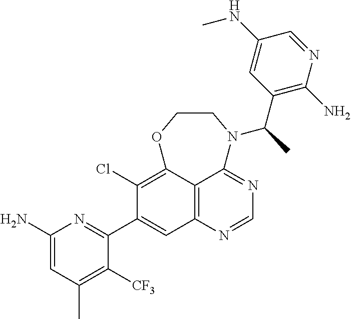 [SMILES: CNc1cnc(N)c([C@@H](C)N2CCOc3c(Cl)c(-c4nc(N)cc(C)c4C(F)(F)F)cc4ncnc2c34)c1] |
| 59 | 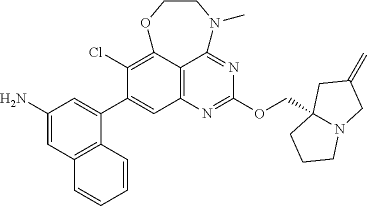 [SMILES: C=C1CN2CCC[C@@]2(COc2nc3c4c(c(Cl)c(-c5cc(N)cc6ccccc56)cc4n2)OCCN3C)C1] |
| 60 | 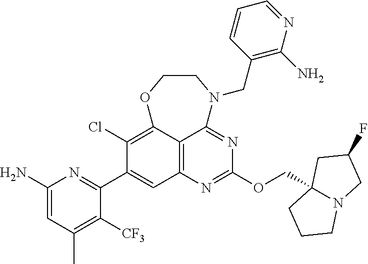 [SMILES: Cc1cc(N)nc(-c2cc3nc(OC[C@@]45CCCN4C[C@H](F)C5)nc4c3c(c2Cl)OCCN4Cc2cccnc2N)c1C(F)(F)F] |
| 61 | 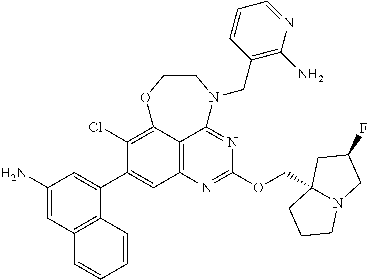 [SMILES: Nc1cc(-c2cc3nc(OC[C@@]45CCCN4C[C@H](F)C5)nc4c3c(c2Cl)OCCN4Cc2cccnc2N)c2ccccc2c1] |
| 62 | 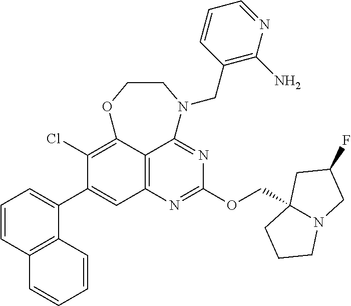 [SMILES: Nc1ncccc1CN1CCOc2c(Cl)c(-c3cccc4ccccc34)cc3nc(OC[C@@]45CCCN4C[C@H](F)C5)nc1c23] |
| 63 | 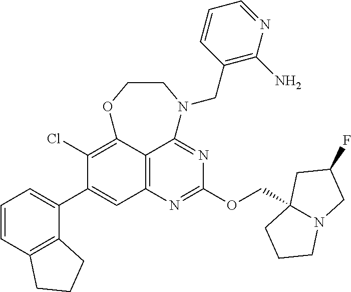 [SMILES: Nc1ncccc1CN1CCOc2c(Cl)c(-c3cccc4c3CCC4)cc3nc(OC[C@@]45CCCN4C[C@H](F)C5)nc1c23] |
| 64 | 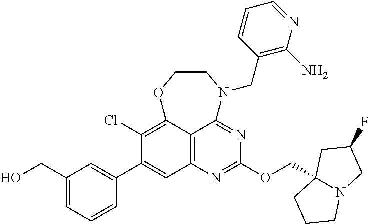 [SMILES: Nc1ncccc1CN1CCOc2c(Cl)c(-c3cccc(CO)c3)cc3nc(OC[C@@]45CCCN4C[C@H](F)C5)nc1c23] |
| 65a | 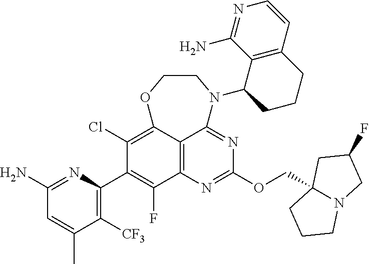 |
| 65b | 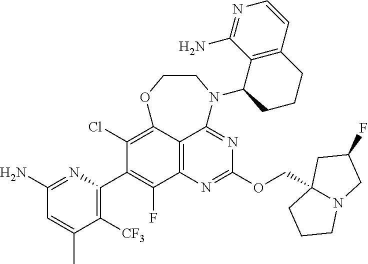 |
| 66a | 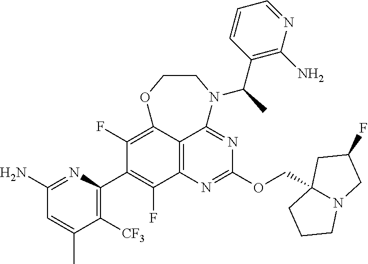 |
| 66b | 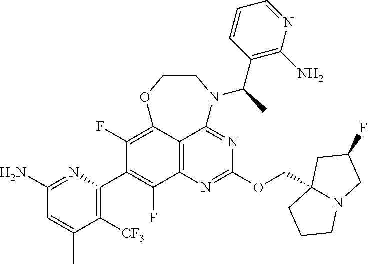 |
| 67 | 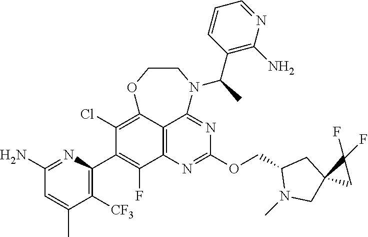 |
| 68 | 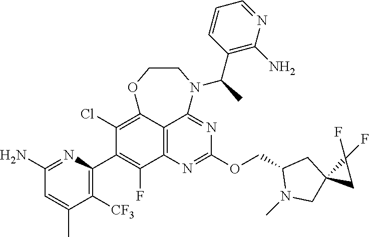 |
| 69a | 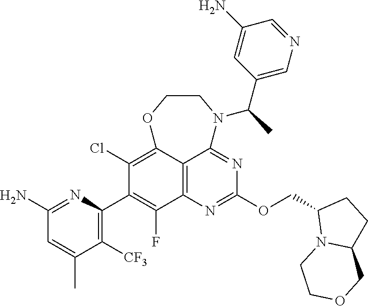 |
| 69b | 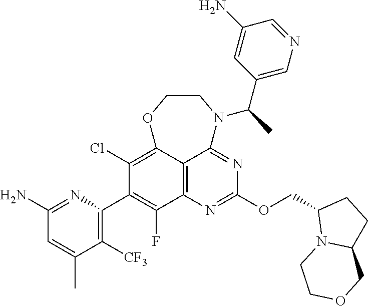 |
| 69c | 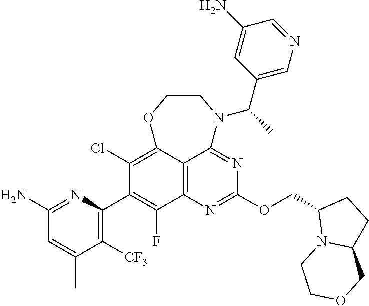 |
| 69d | 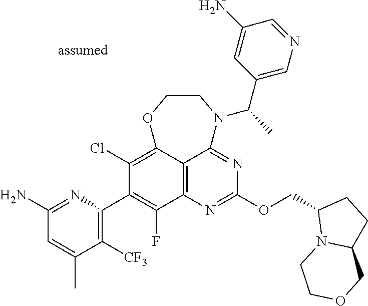 |
| 70a | 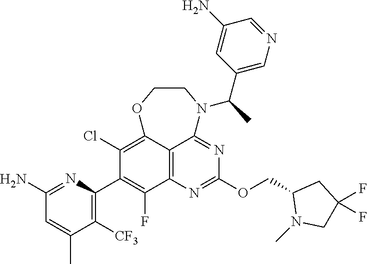 |
| 70b | 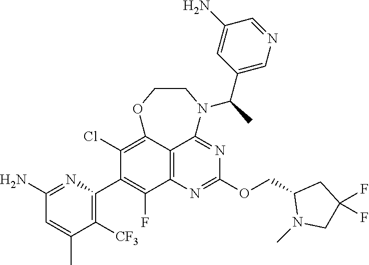 |
| 70c | 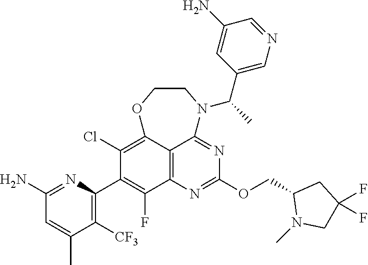 |
| 70d | 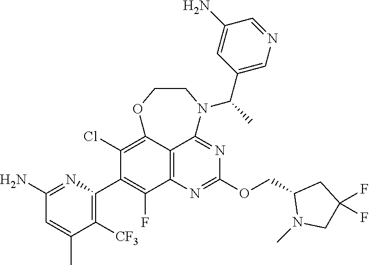 |
| 71 | 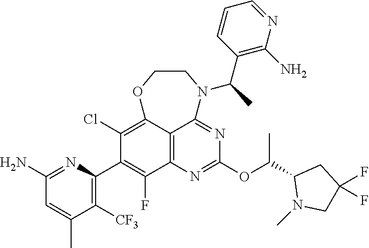 |
| 72 | 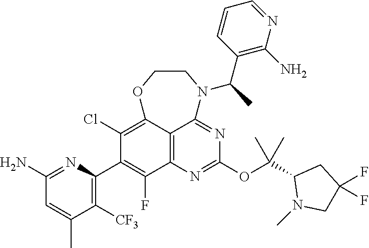 |
| 73a | 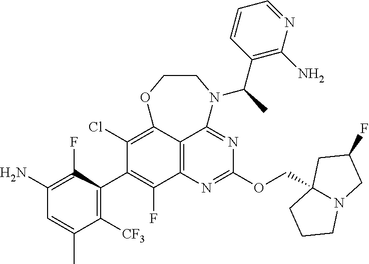 |
| 73b | 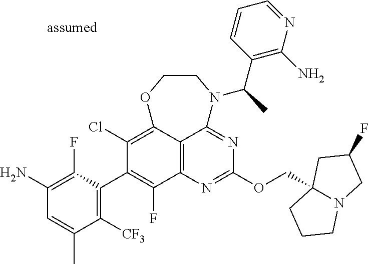 |
| 74a | 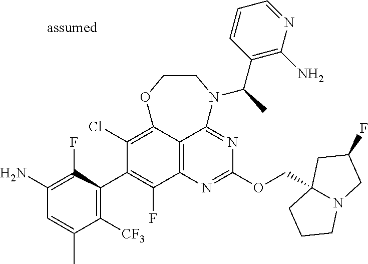 |
| 74b | 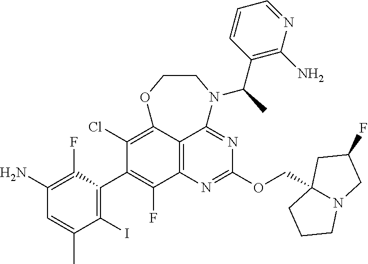 |
| 75a | 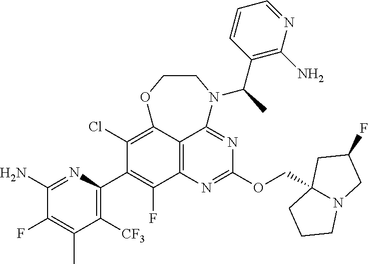 |
| 75b | 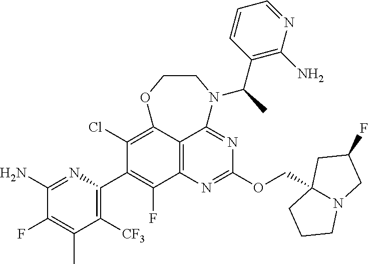 |
| 76a | 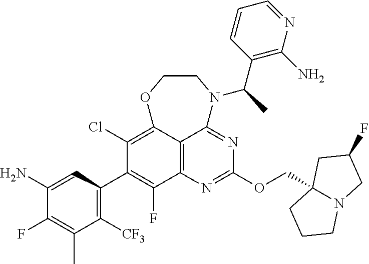 |
| 76b | 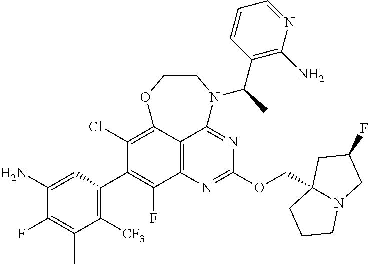 |
| 77a | 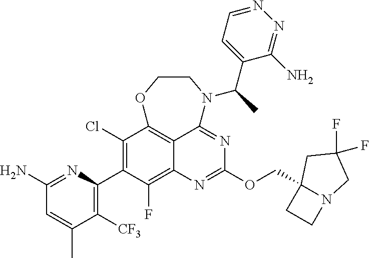 |
| 77b | 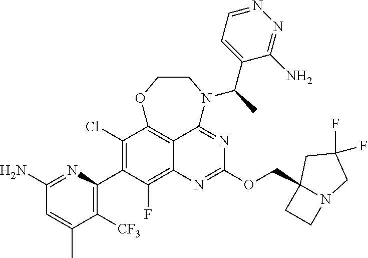 |
| 77c | 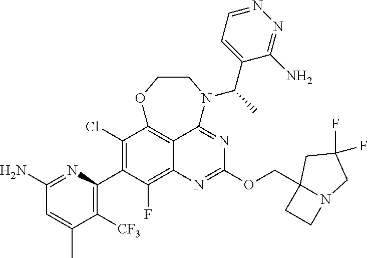 |
| 78 | 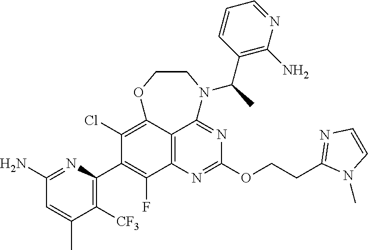 |
| 79 | 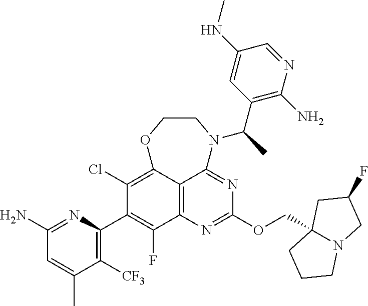 |
| 80 | 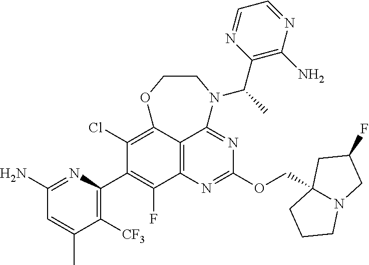 |
| 81 | 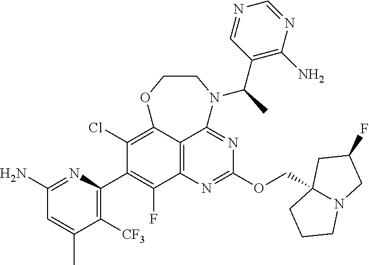 |
| 82a | 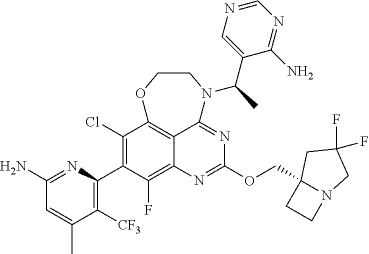 |
| 82b | 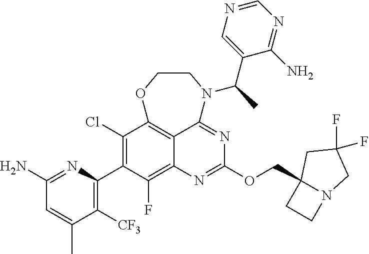 |
| 83a | 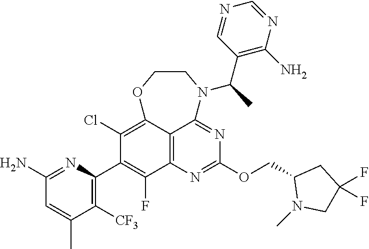 |
| 83b | 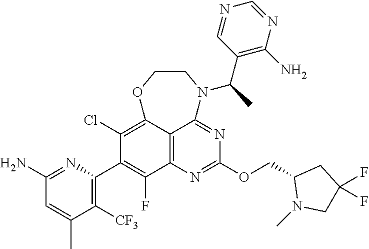 |
| 84a | 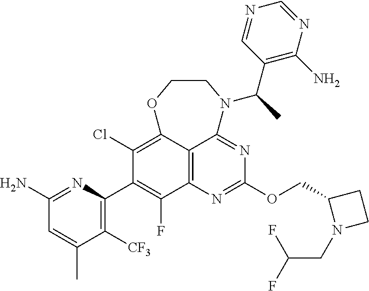 |
| 84b | 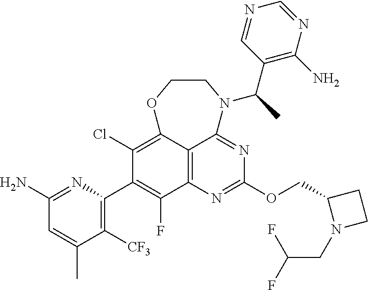 |
| 85 | 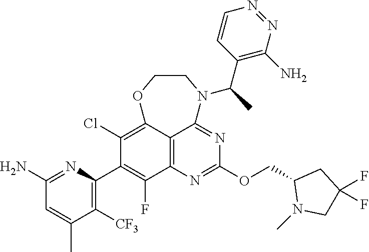 |
| 86 | 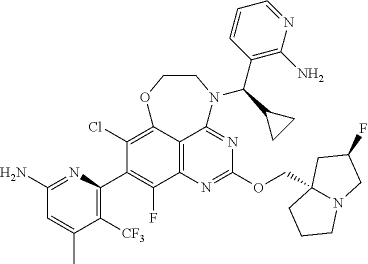 |
| 87a | 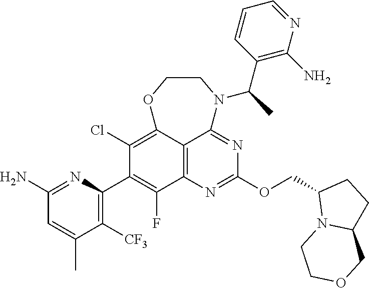 |
| 87b | 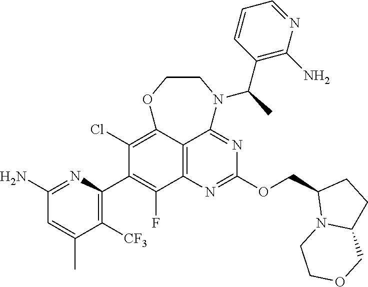 |
| 88 | 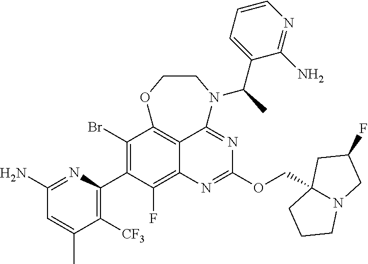 |
| 89 | 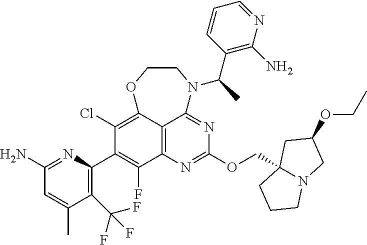 |
| 90 | 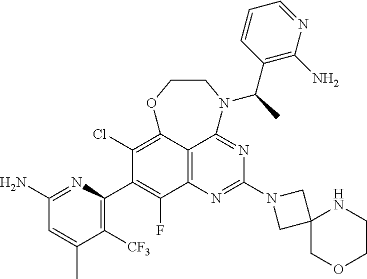 |
| 91a | 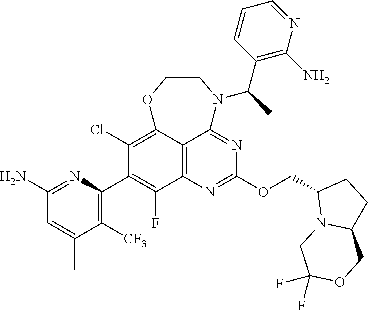 |
| 91b | 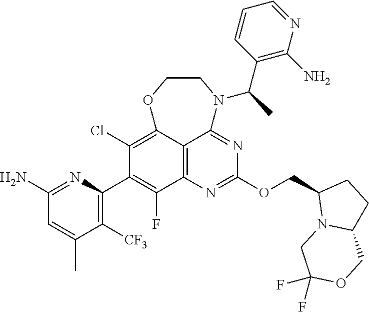 |
| 92a | 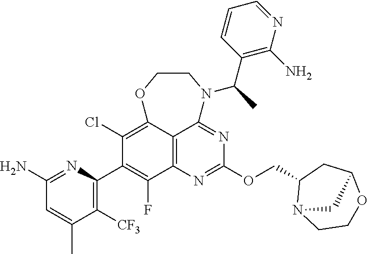 |
| 92b | 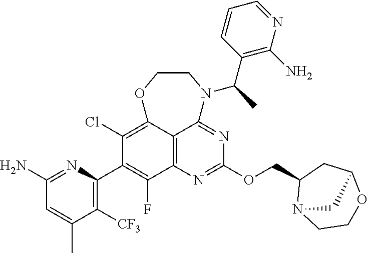 |
| 93a | 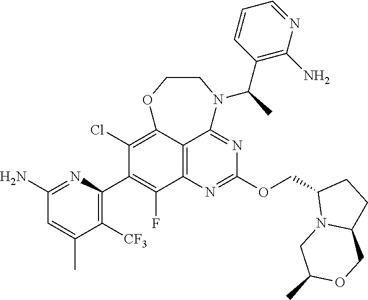 |
| 93b | 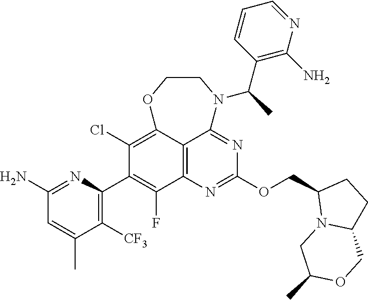 |
| 93c | 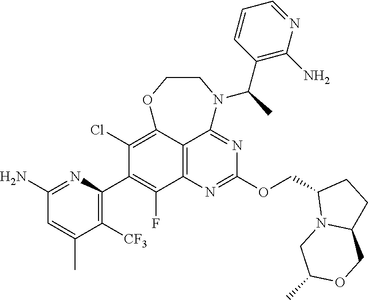 |
| 93d | 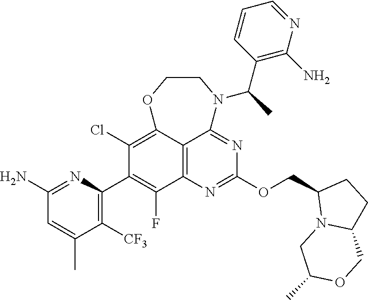 |
| 94 | 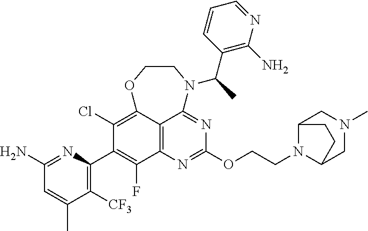 |
| 95a | 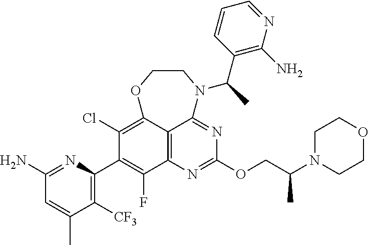 |
| 95b | 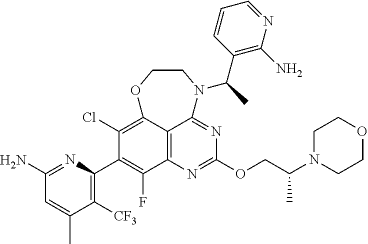 |
| 96 | 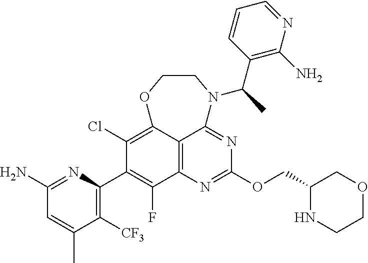 |
| 97 | 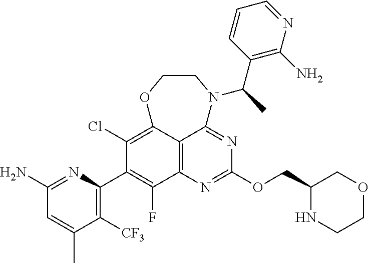 |
| 98a | 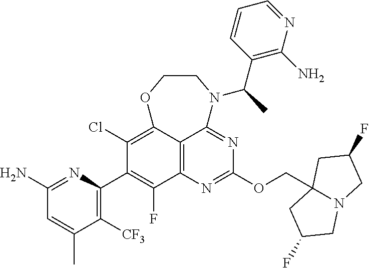 |
| 98b | 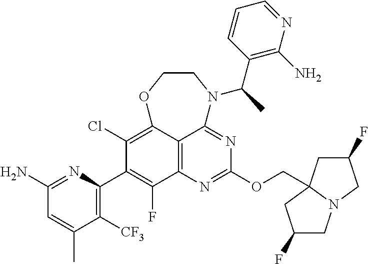 |
| 99a | 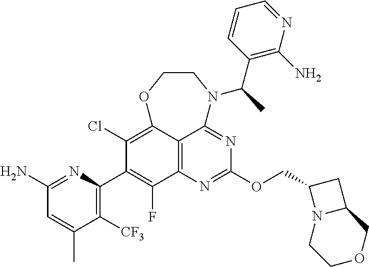 |
| 99b | 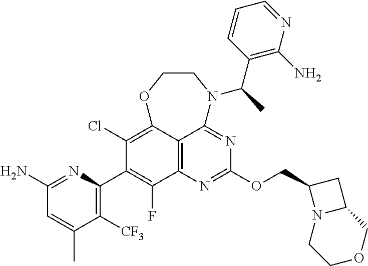 |
| 100a | 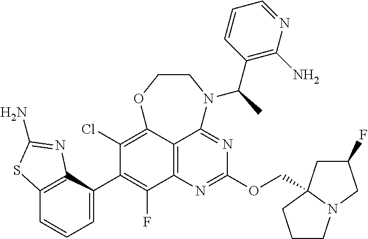 |
| 100b | 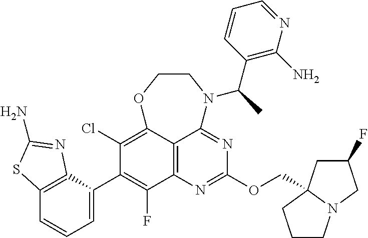 |
| 101 | 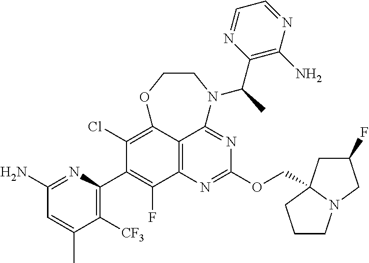 |
| 102a | 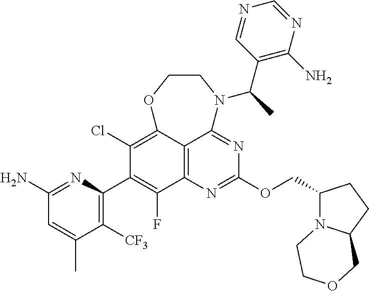 |
| 102b | 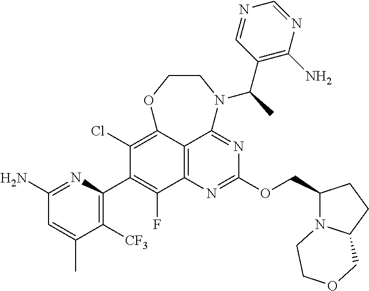 |
| 102c | 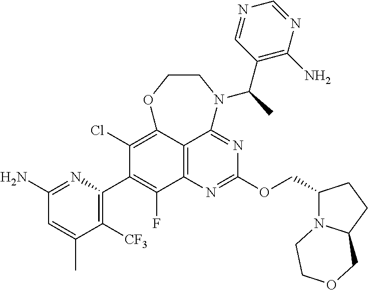 |
| 102d | 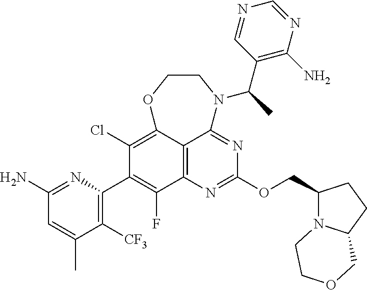 |
| 103a | 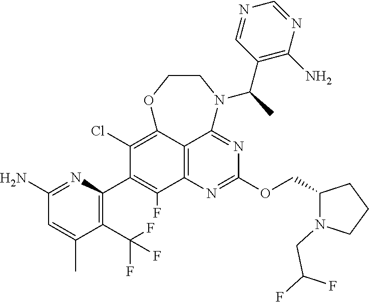 |
| 103b | 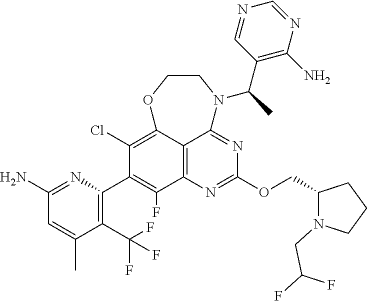 |
| 104a | 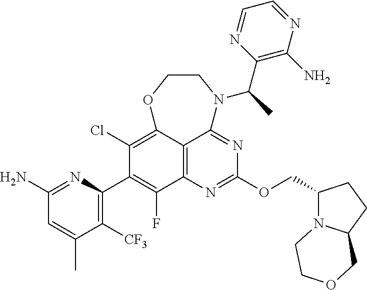 |
| 104b | 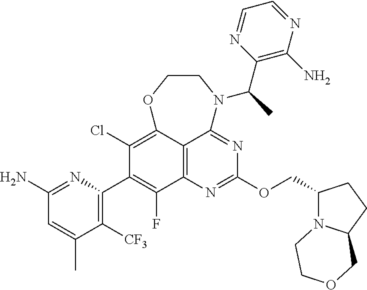 |
| 105a | 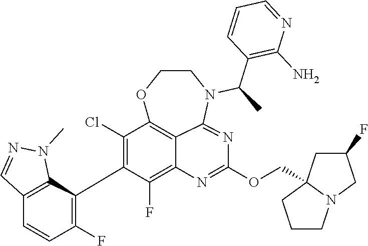 |
| 105b | 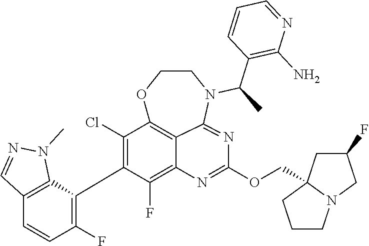 |
| 106 | 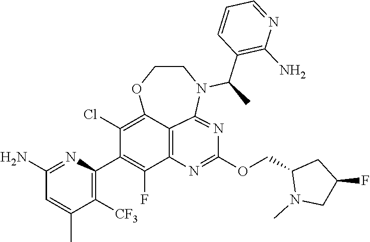 |
| 107 | 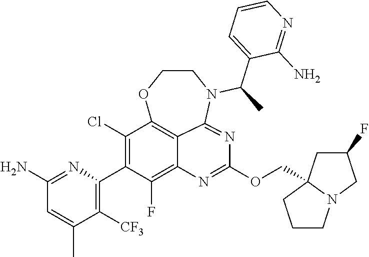 |
| 108a | 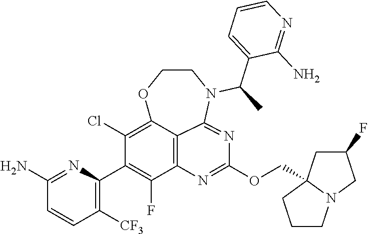 |
| 108b | 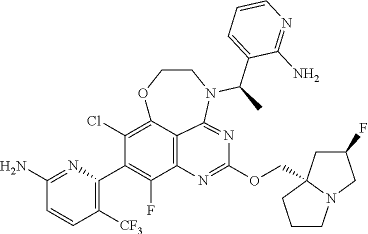 |
| 109 | 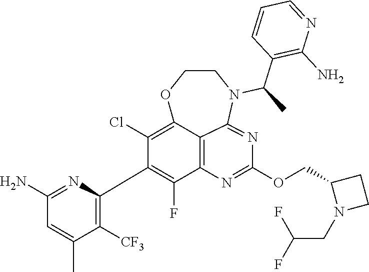 |
| 110a | 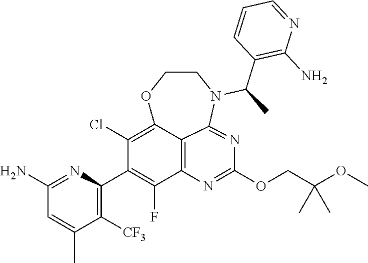 |
| 110b | 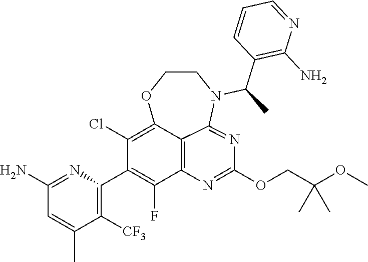 |
| 111a | 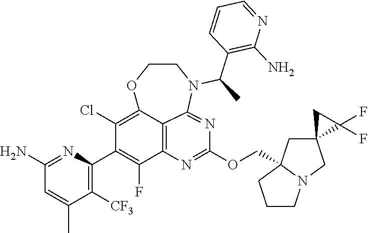 |
| 111b | 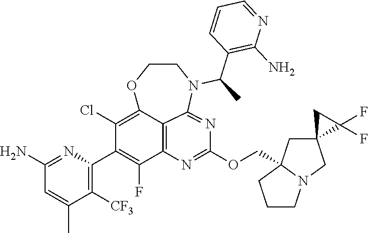 |
| 112 | 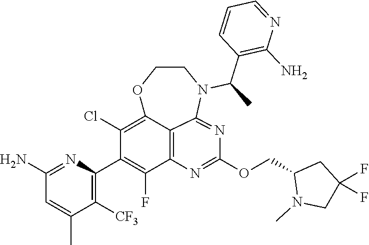 |
| 113a | 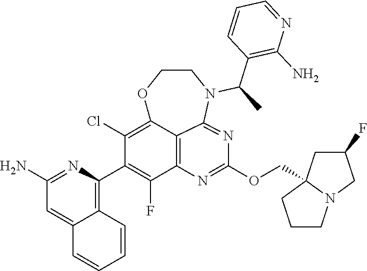 |
| 113b | 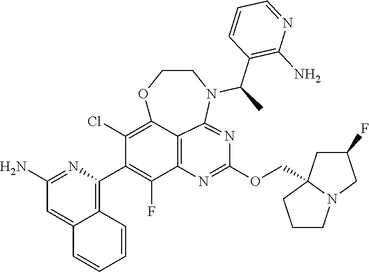 |
| 114a | 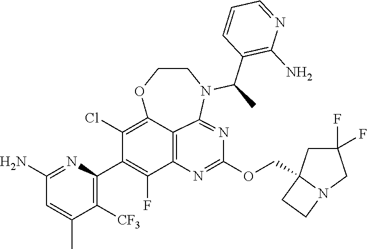 |
| 114b | 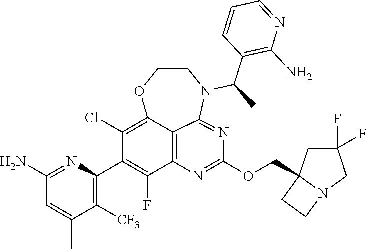 |
| 115 | 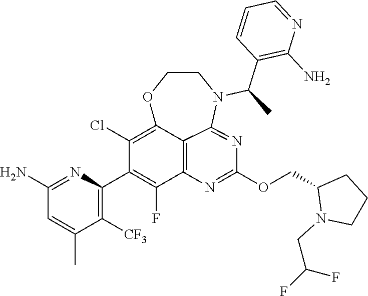 |
| 116 | 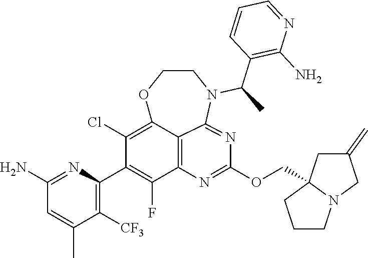 |
| 117 | 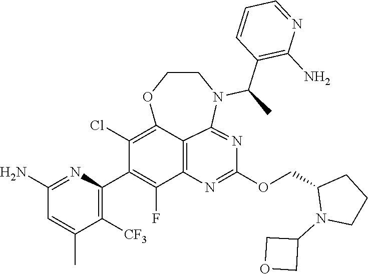 |
| 118 | 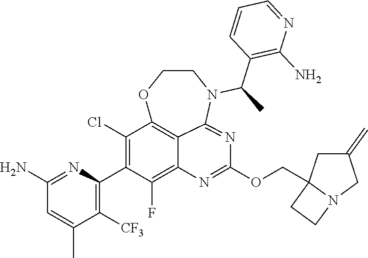 |
| 119 | 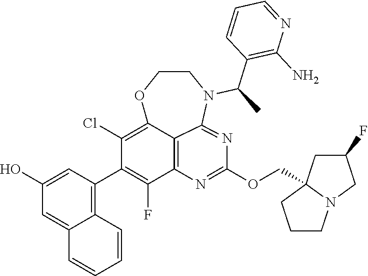 [SMILES: C[C@H](c1cccnc1N)N1CCOc2c(Cl)c(-c3cc(O)cc4ccccc34)c(F)c3nc(OC[C@@]45CCCN4C[C@H](F)C5)nc1c23] |
| 120a | 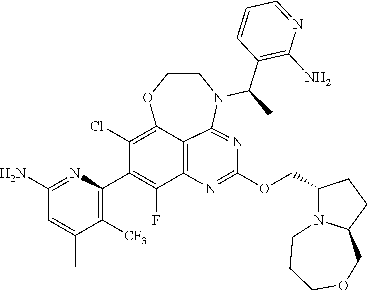 |
| 120b | 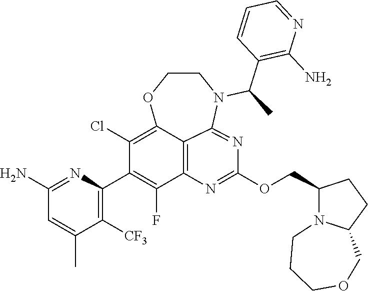 |
| 121 | 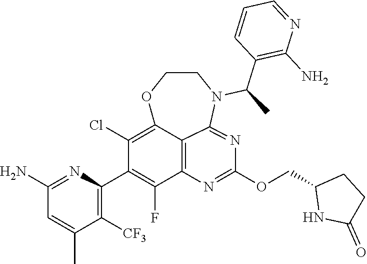 |
| 122a | 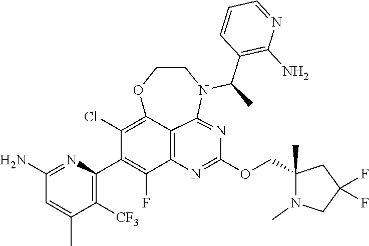 |
| 122b | 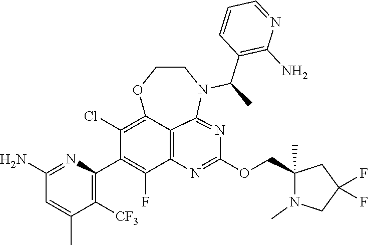 |
| 123a | 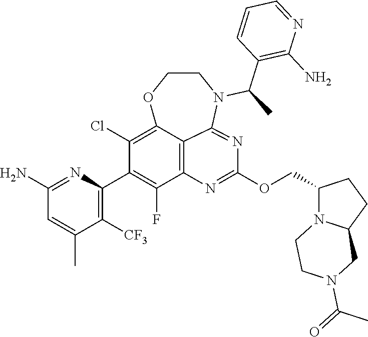 |
| 123 | 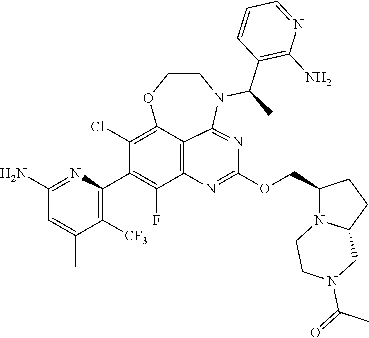 |
| 124 | 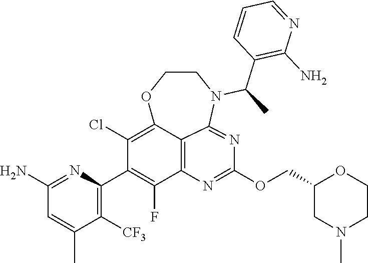 |
| 125a | 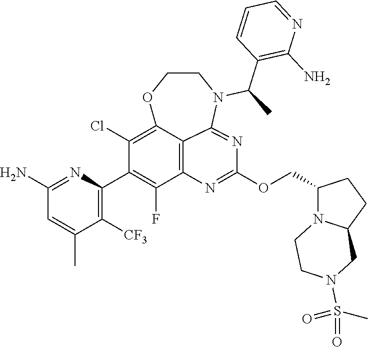 |
| 125b | 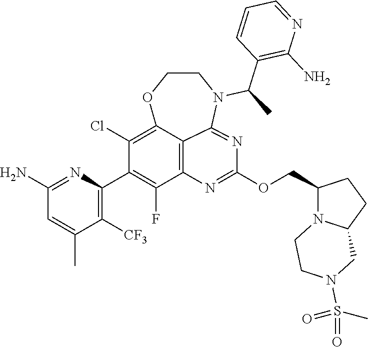 |
| 126 | 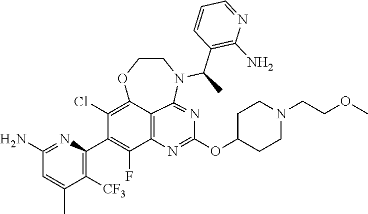 |
| 127 | 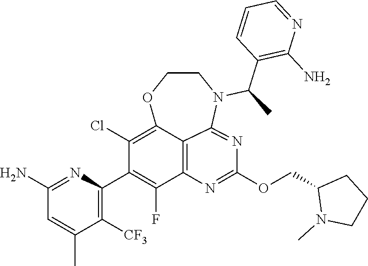 |
| 128a | 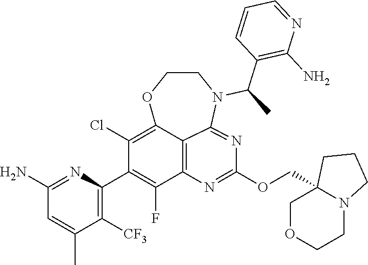 |
| 128b | 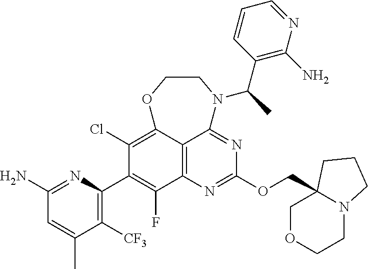 |
| 129a | 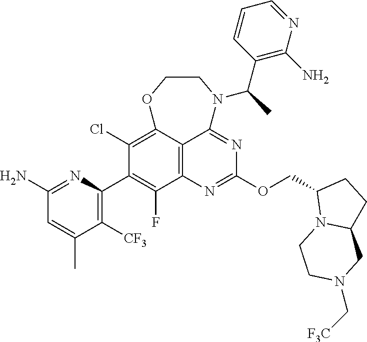 |
| 129b | 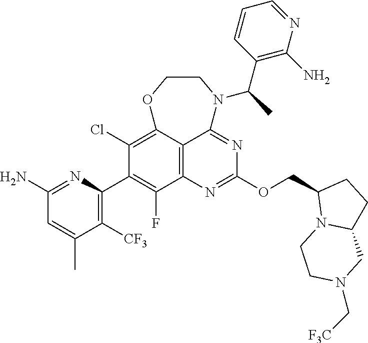 |
| 130a | 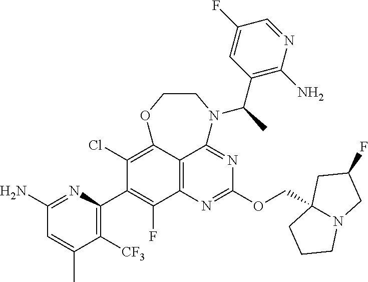 |
| 130b | 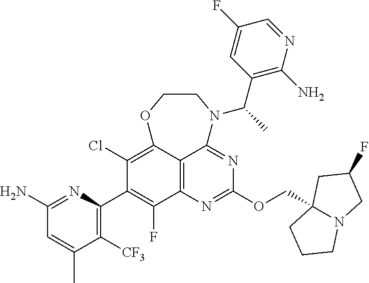 |
| 130c | 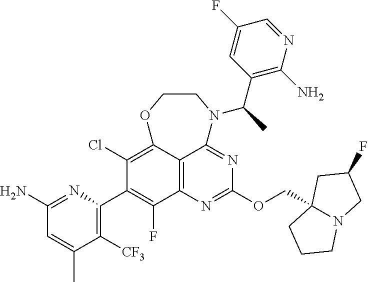 |
| 130d | 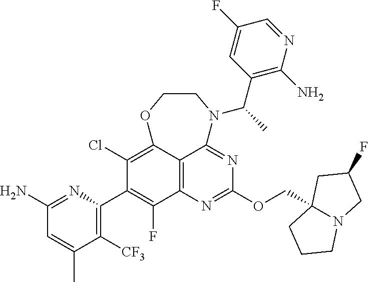 |
| 131 | 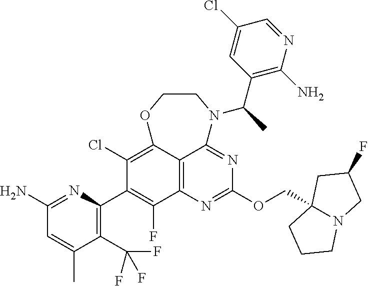 |
| 132 | 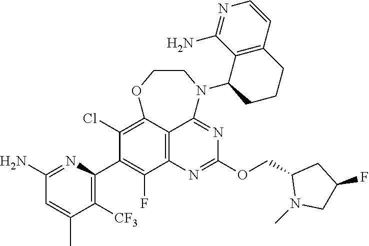 |
| 133a | 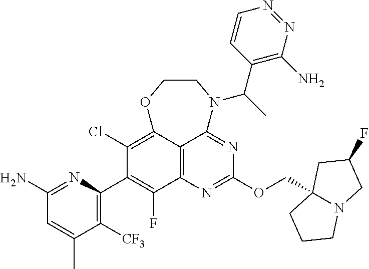 |
| 133b | 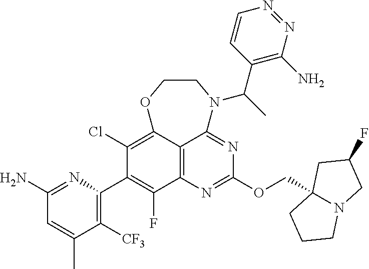 |
| 133c | 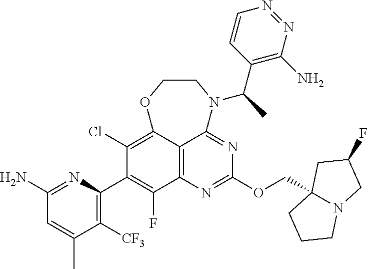 |
| 134 | 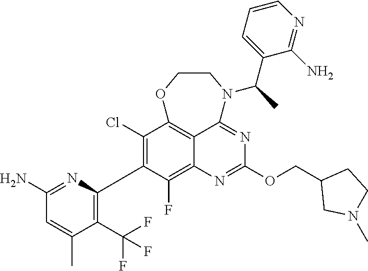 |
| 135 | 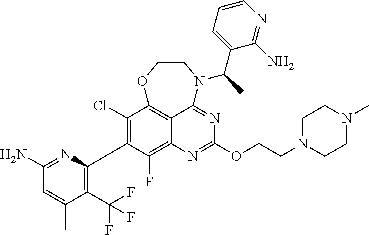 |
| 136 | 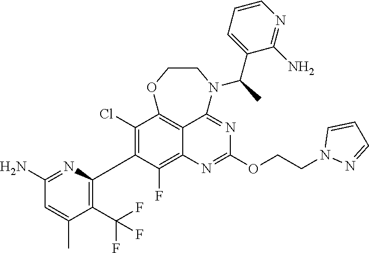 |
| 137 | 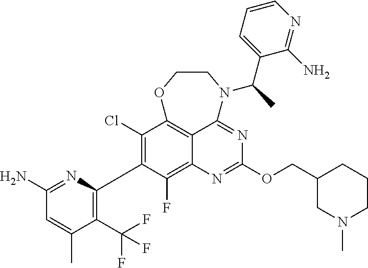 |
| 138 | 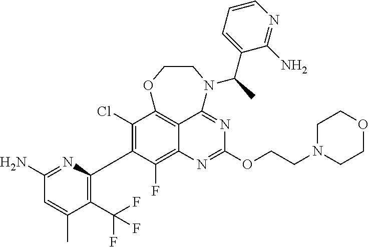 |
| 139 | 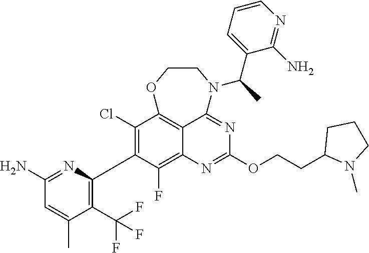 |
| 140 | 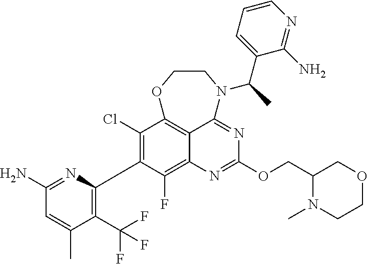 |
| 141 | 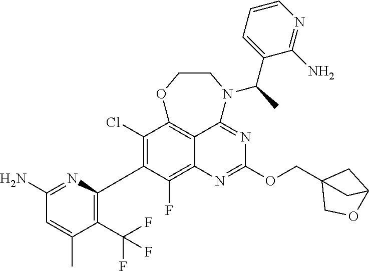 |
| 142 | 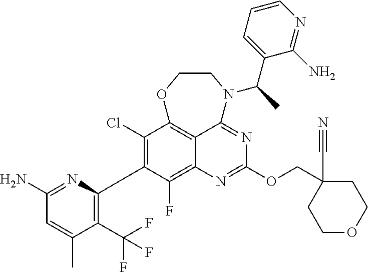 |
| 143 | 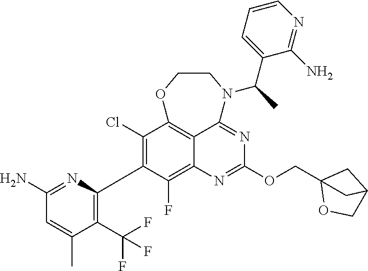 |
| 144 | 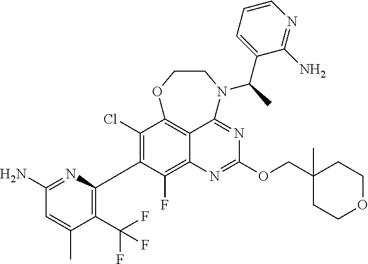 |
| 145 | 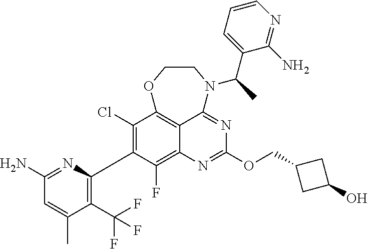 |
| 146 | 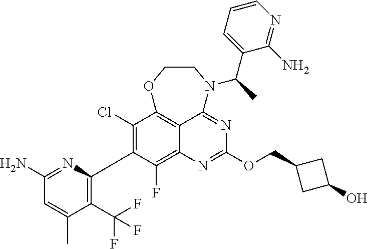 |
| 147 | 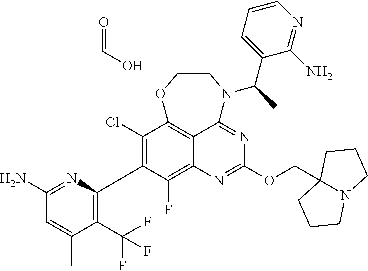 |
| 148 | 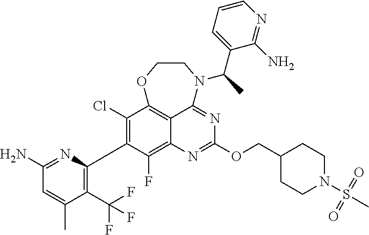 |
| 149 | 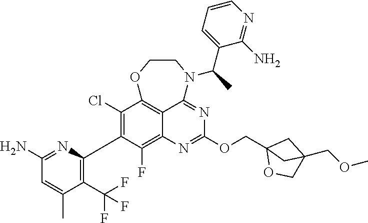 |
| 150 | 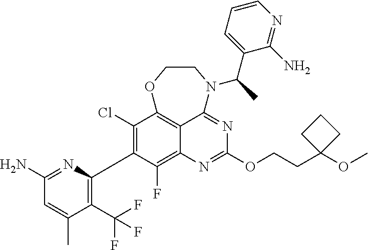 |
| 151 | 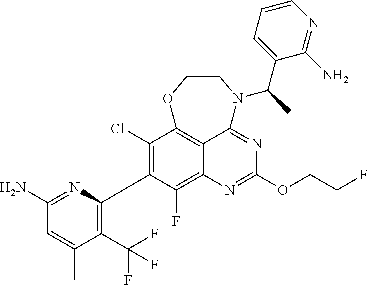 |
| 152 | 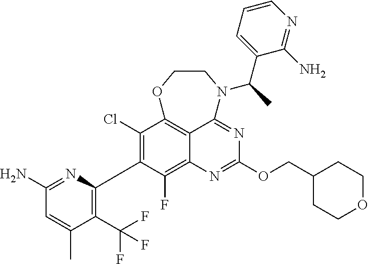 |
| 153 | 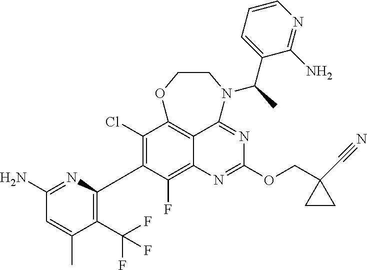 |
| 154 | 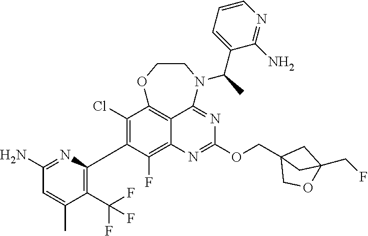 |
| 155 | 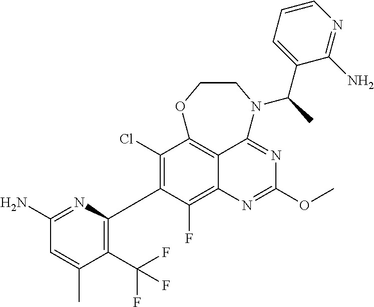 |
| 156 | 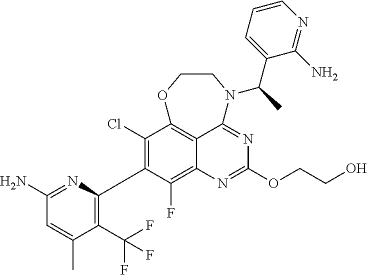 |
| 157 | 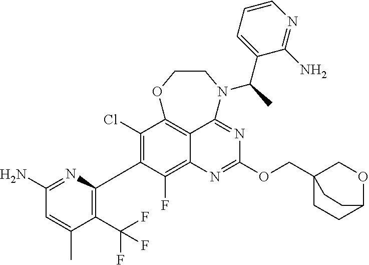 |
| 158a | 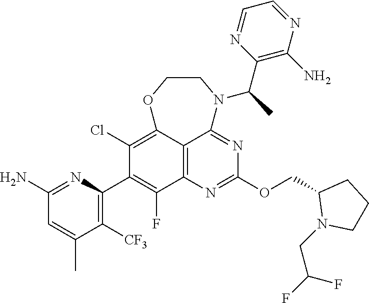 |
| 158b | 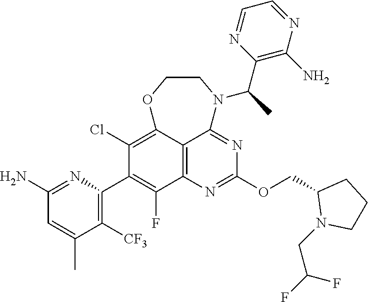 |
| 159a | 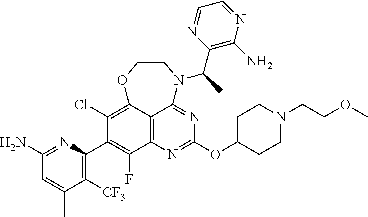 |
| 159b | 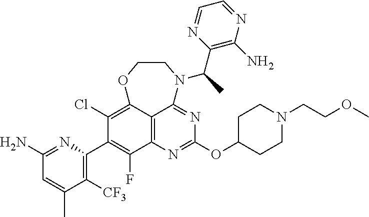 |
| 160a | 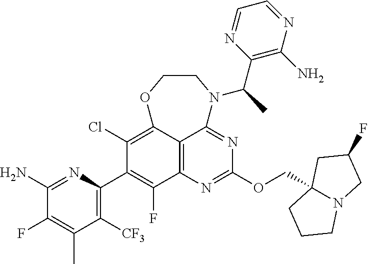 |
| 160b | 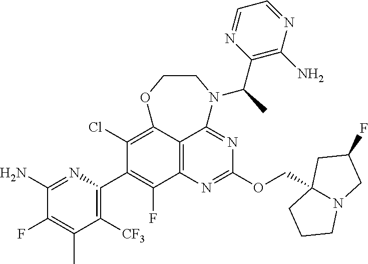 |
| 161a | 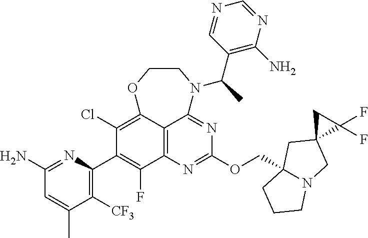 |
| 161b | 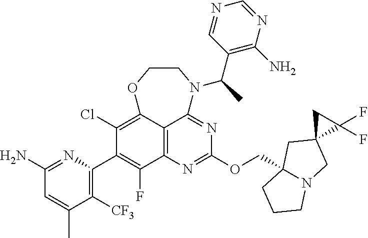 |
| 162a | 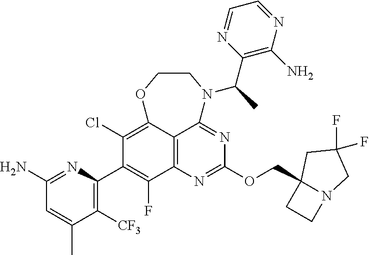 |
| 162b | 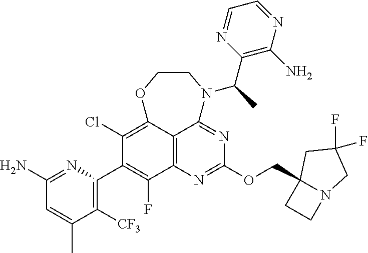 |
| 162c | 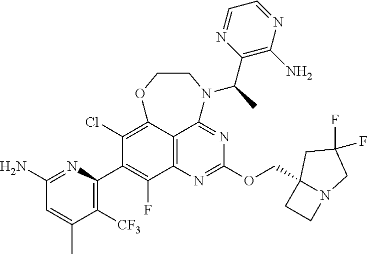 |
| 162d | 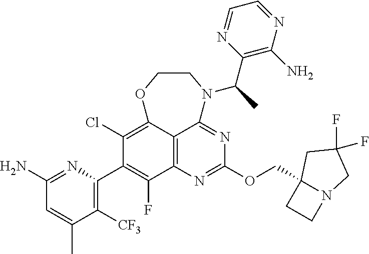 |
| 163a | 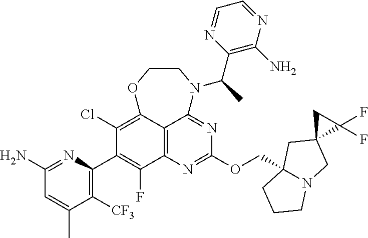 |
| 163b | 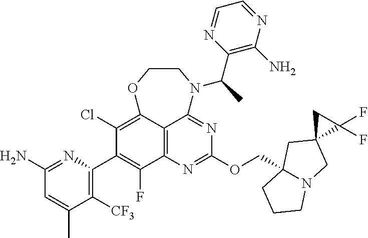 |
| 164a | 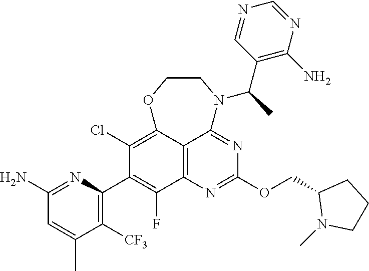 |
| 164b | 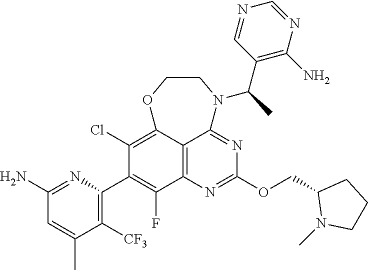 |
| 165a | 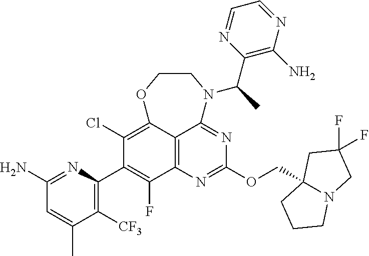 |
| 165b | 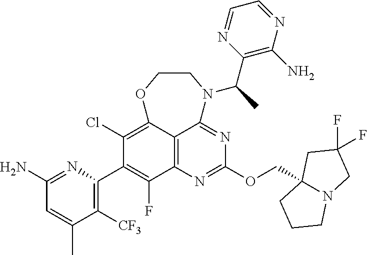 |
| 166a | 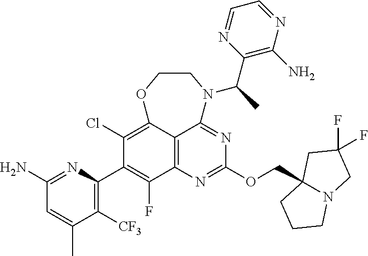 |
| 166b | 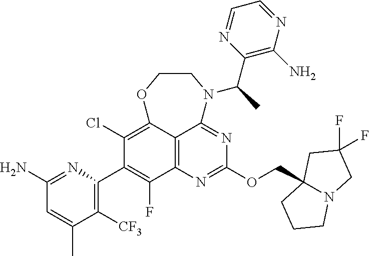 |
| 167a | 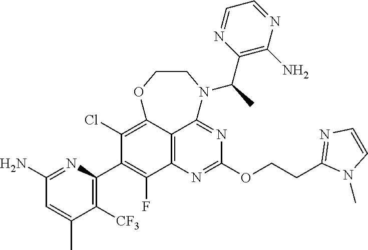 |
| 167b | 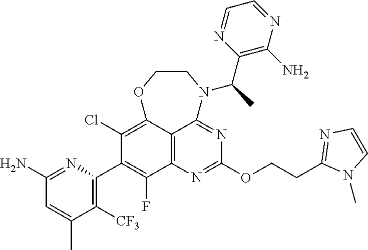 |
| 168a | 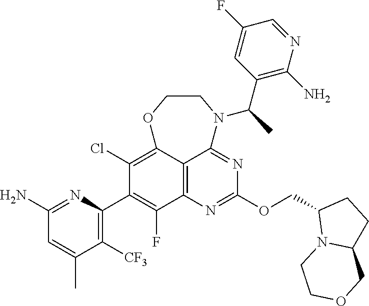 |
| 168b | 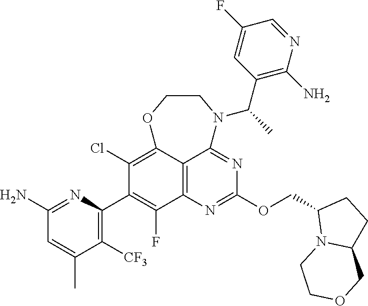 |
| 169a | 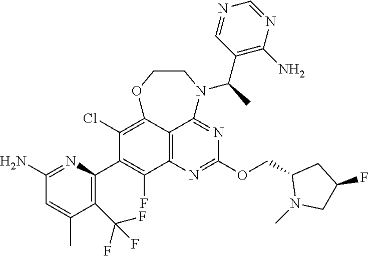 |
| 169b | 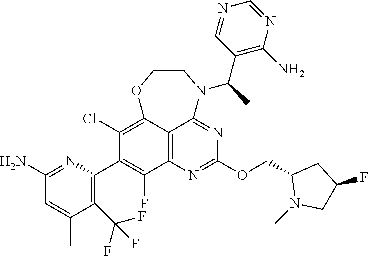 |
| 170a | 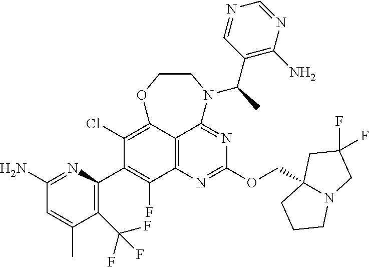 |
| 170b | 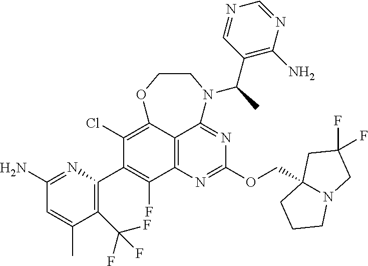 |
| 171a | 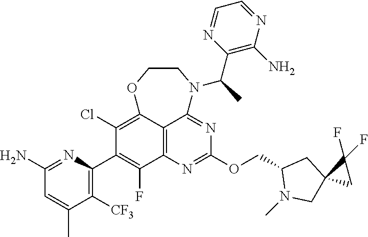 |
| 171b | 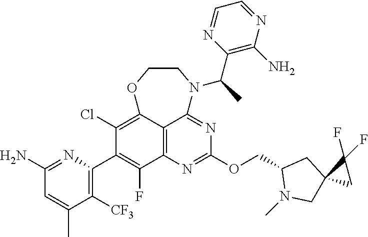 |
| 172a | 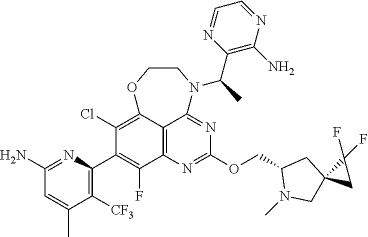 |
| 172b | 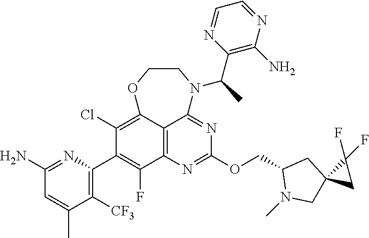 |
| 173a | 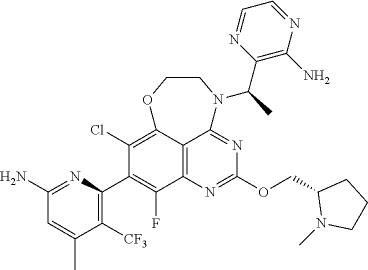 |
| 173b | 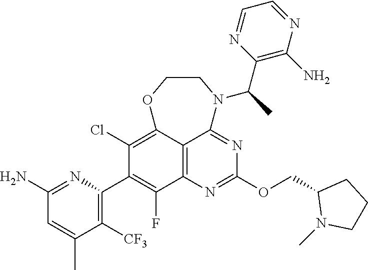 |
| 174a | 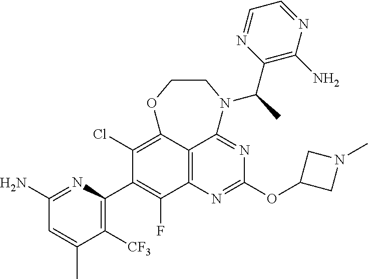 |
| 174b | 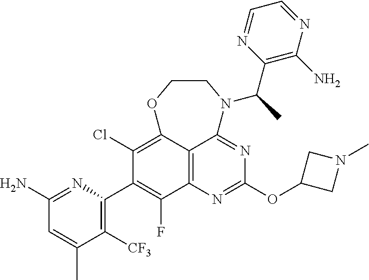 |
| 175 | 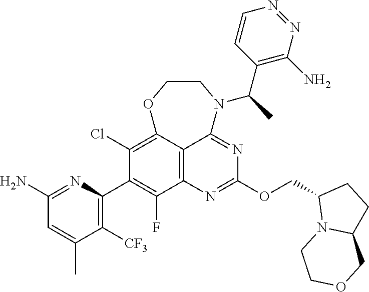 |
| 176 | 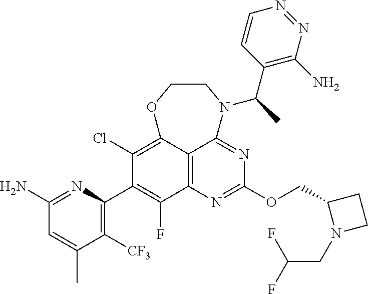 |

In one embodiment, the compound of formula (I), (II), or (III) or a stereoisomer, atropisomer, tautomer, or pharmaceutically acceptable salt thereof is a compound of Table 1.

TABLE 2
G12D Compounds

| Ex. | |
|---|---|
| No. | Structure |
| 501 | 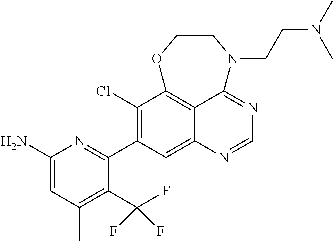 [SMILES: Cc1cc(N)nc(-c2cc3ncnc4c3c(c2Cl)OCCN4CCN(C)C)c1C(F)(F)F] |
| 502 | 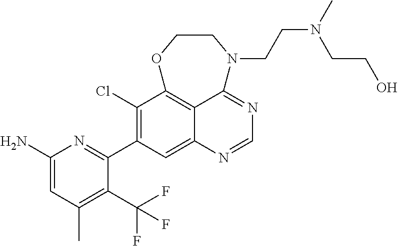 [SMILES: Cc1cc(N)nc(-c2cc3ncnc4c3c(c2Cl)OCCN4CCN(C)CCO)c1C(F)(F)F] |
| 503 | 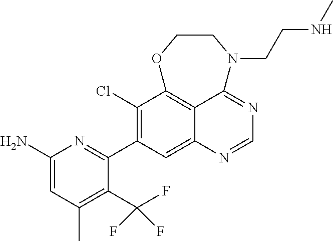 [SMILES: CNCCN1CCOc2c(Cl)c(-c3nc(N)cc(C)c3C(F)(F)F)cc3ncnc1c23] |
| 504 | 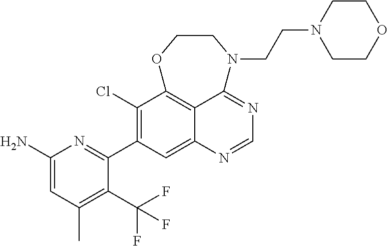 [SMILES: Cc1cc(N)nc(-c2cc3ncnc4c3c(c2Cl)OCCN4CCN2CCOCC2)c1C(F)(F)F] |
| 505 | 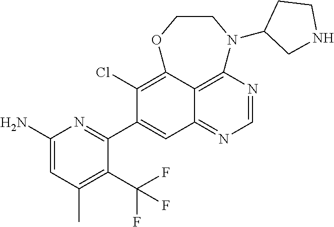 [SMILES: Cc1cc(N)nc(-c2cc3ncnc4c3c(c2Cl)OCCN4C2CCNC2)c1C(F)(F)F] |
| 506 | 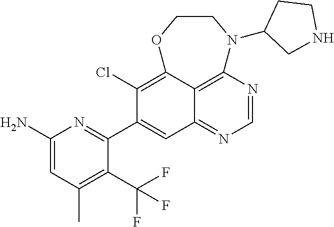 [SMILES: Cc1cc(N)nc(-c2cc3ncnc4c3c(c2Cl)OCCN4C2CCNC2)c1C(F)(F)F] |
| 507 | 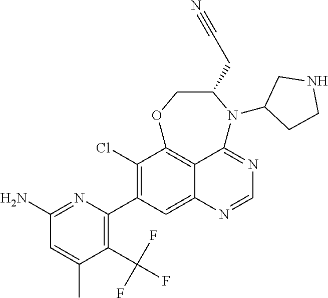 [SMILES: Cc1cc(N)nc(-c2cc3ncnc4c3c(c2Cl)OC[C@H](CC#N)N4C2CCNC2)c1C(F)(F)F] |
| 508 | 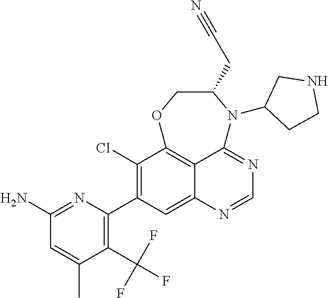 [SMILES: Cc1cc(N)nc(-c2cc3ncnc4c3c(c2Cl)OC[C@H](CC#N)N4C2CCNC2)c1C(F)(F)F] |
| 509 | 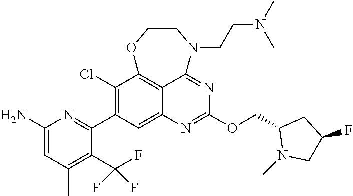 [SMILES: Cc1cc(N)nc(-c2cc3nc(OC[C@@H]4C[C@@H](F)CN4C)nc4c3c(c2Cl)OCCN4CCN(C)C)c1C(F)(F)F] |
| 510 | 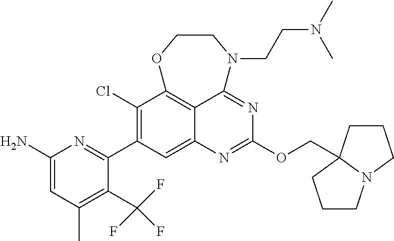 [SMILES: Cc1cc(N)nc(-c2cc3nc(OCC45CCCN4CCC5)nc4c3c(c2Cl)OCCN4CCN(C)C)c1C(F)(F)F] |
| 511 | 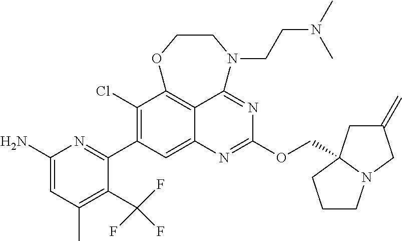 [SMILES: C=C1CN2CCC[C@@]2(COc2nc3c4c(c(Cl)c(-c5nc(N)cc(C)c5C(F)(F)F)cc4n2)OCCN3CCN(C)C)C1] |
| 512 | 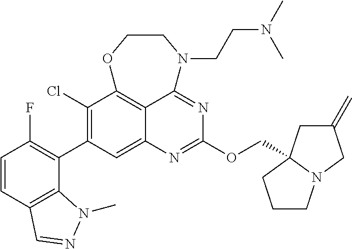 [SMILES: C=C1CN2CCC[C@@]2(COc2nc3c4c(c(Cl)c(-c5c(F)ccc6cnn(C)c56)cc4n2)OCCN3CCN(C)C)C1] |
| 513 | 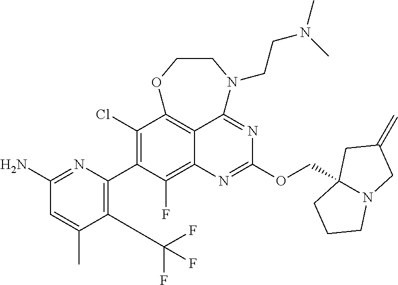 [SMILES: C=C1CN2CCC[C@@]2(COc2nc3c4c(c(Cl)c(-c5nc(N)cc(C)c5C(F)(F)F)c(F)c4n2)OCCN3CCN(C)C)C1] |
| 514 | 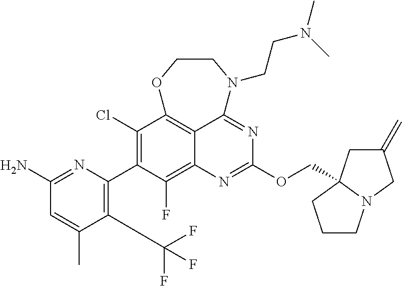 [SMILES: C=C1CN2CCC[C@@]2(COc2nc3c4c(c(Cl)c(-c5nc(N)cc(C)c5C(F)(F)F)c(F)c4n2)OCCN3CCN(C)C)C1] |
| 515 | 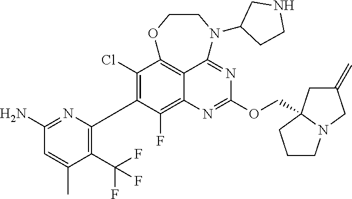 [SMILES: C=C1CN2CCC[C@@]2(COc2nc3c4c(c(Cl)c(-c5nc(N)cc(C)c5C(F)(F)F)c(F)c4n2)OCCN3C2CCNC2)C1] |
| 516 | 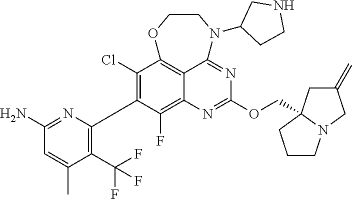 [SMILES: C=C1CN2CCC[C@@]2(COc2nc3c4c(c(Cl)c(-c5nc(N)cc(C)c5C(F)(F)F)c(F)c4n2)OCCN3C2CCNC2)C1] |
| 517 | 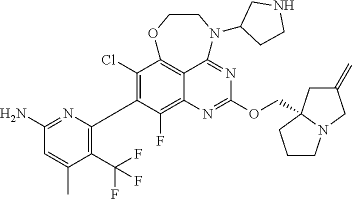 [SMILES: C=C1CN2CCC[C@@]2(COc2nc3c4c(c(Cl)c(-c5nc(N)cc(C)c5C(F)(F)F)c(F)c4n2)OCCN3C2CCNC2)C1] |
| 518 | 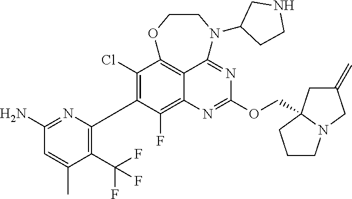 [SMILES: C=C1CN2CCC[C@@]2(COc2nc3c4c(c(Cl)c(-c5nc(N)cc(C)c5C(F)(F)F)c(F)c4n2)OCCN3C2CCNC2)C1] |
| 519 | 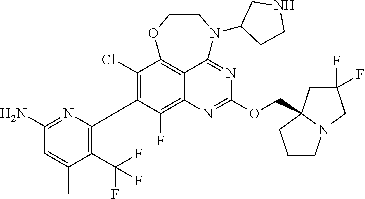 [SMILES: Cc1cc(N)nc(-c2c(Cl)c3c4c(nc(OC[C@]56CCCN5CC(F)(F)C6)nc4c2F)N(C2CCNC2)CCO3)c1C(F)(F)F] |
| 520 | 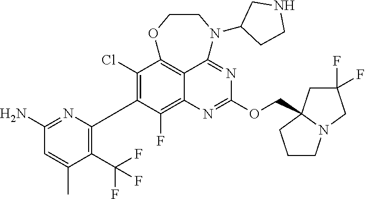 [SMILES: Cc1cc(N)nc(-c2c(Cl)c3c4c(nc(OC[C@]56CCCN5CC(F)(F)C6)nc4c2F)N(C2CCNC2)CCO3)c1C(F)(F)F] |
| 521 | 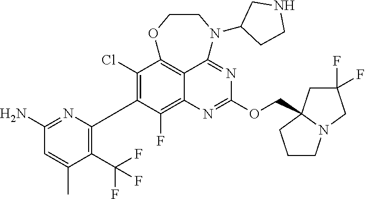 [SMILES: Cc1cc(N)nc(-c2c(Cl)c3c4c(nc(OC[C@]56CCCN5CC(F)(F)C6)nc4c2F)N(C2CCNC2)CCO3)c1C(F)(F)F] |
| 522 | 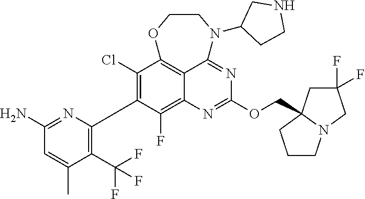 [SMILES: Cc1cc(N)nc(-c2c(Cl)c3c4c(nc(OC[C@]56CCCN5CC(F)(F)C6)nc4c2F)N(C2CCNC2)CCO3)c1C(F)(F)F] |
| 523 | 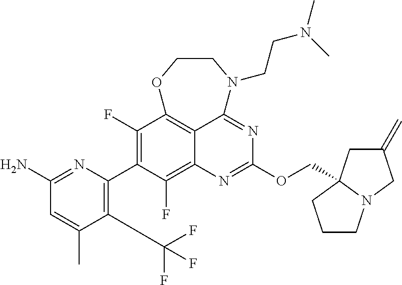 [SMILES: C=C1CN2CCC[C@@]2(COc2nc3c4c(c(F)c(-c5nc(N)cc(C)c5C(F)(F)F)c(F)c4n2)OCCN3CCN(C)C)C1] |
| 524 | 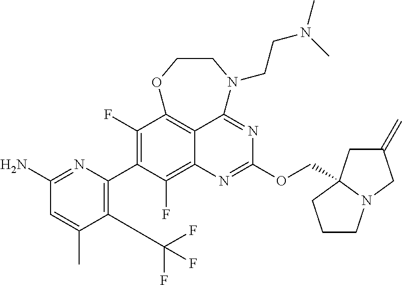 [SMILES: C=C1CN2CCC[C@@]2(COc2nc3c4c(c(F)c(-c5nc(N)cc(C)c5C(F)(F)F)c(F)c4n2)OCCN3CCN(C)C)C1] |
| 525 | 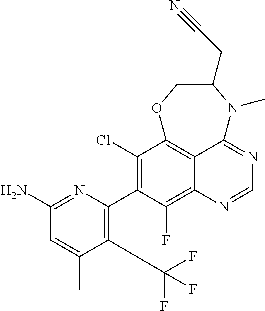 [SMILES: Cc1cc(N)nc(-c2c(Cl)c3c4c(ncnc4c2F)N(C)C(CC#N)CO3)c1C(F)(F)F] |
| 525a | 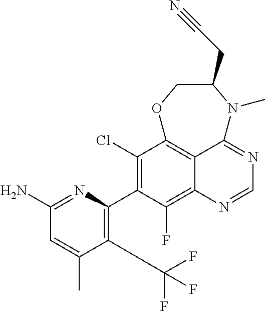 |
| 525b | 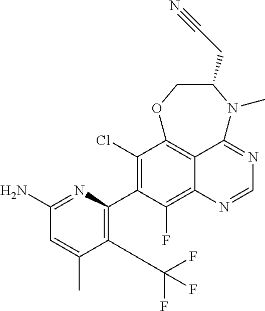 |
| 525c | 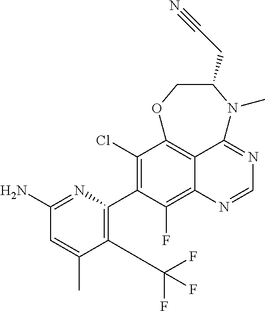 |
| 525d | 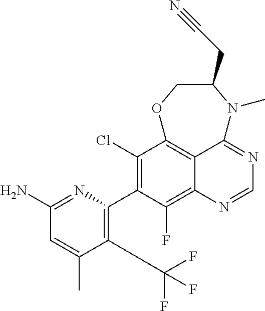 |

### Synthesis of Compounds

Compounds or a stereoisomer, atropisomer, tautomer, or pharmaceutically acceptable salt thereof as described herein of the present disclosure can be made by a variety of methods depicted in the illustrative synthetic reaction schemes shown and described below. The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, vol. 1-21; R. C. LaRock, Comprehensive Organic Transformations, 2nd edition Wiley-VCH, New York 1999; Comprehensive Organic Synthesis, B. Trost and I. Fleming (Eds.) vol. 1-9 Pergamon, Oxford, 1991; Comprehensive Heterocyclic Chemistry, A. R. Katritzky and C. W. Rees (Eds.) Pergamon, Oxford 1984, vol. 1-9; Comprehensive Heterocyclic Chemistry II, A. R. Katritzky and C. W. Rees (Eds) Pergamon, Oxford 1996, vol. 1-11; and Organic Reactions, Wiley & Sons: New York, 1991, vol. 1-40. The synthetic reaction schemes provided herein are merely illustrative of some methods by which the compounds or pharmaceutical acceptable salts thereof described herein can be synthesized, and various modifications to these synthetic reaction schemes can be made and will be suggested to one skilled in the art having referred to the disclosure contained herein.

Synthetic chemistry transformations and protecting group methodologies (protection and deprotection) useful in synthesizing compounds described herein and necessary reagents and intermediates include, for example, those described in R. Larock, Comprehensive Organic Transformations, VCH Publishers (1989); T. W. Greene and P. G. M. Wuts, Protective Groups in Organic Synthesis, 3^{r} Ed., John Wiley and Sons (1999); and L. Paquette, ed., Encyclopedia of Reagents for Organic Synthesis, John Wiley and Sons (1995) and subsequent editions thereof.

Compounds or a stereoisomer, atropisomer, tautomer, or pharmaceutically acceptable salt thereof as described herein described herein can be prepared singly or as compound libraries comprising at least 2, for example 5 to 1,000 compounds, or 10 to 100 compounds. Libraries of compounds or a stereoisomer, atropisomer, tautomer, or pharmaceutically acceptable salt thereof as described herein of the formulae described herein can be prepared by a combinatorial split and mix approach or by multiple parallel syntheses using, for example, either solution phase or solid phase chemistry. Thus according to a further aspect provided herein is a compound library comprising at least 2 compounds or a stereoisomer, atropisomer, tautomer, or pharmaceutically acceptable salt thereof as described herein.

The Examples provide exemplary methods for preparing compounds or a stereoisomer, atropisomer, tautomer, or pharmaceutically acceptable salt thereof as described herein. Those skilled in the art will appreciate that other synthetic routes can be used to synthesize the compounds or a stereoisomer, atropisomer, tautomer, or pharmaceutically acceptable salt thereof as described herein described herein. Although specific starting materials and reagents are depicted and discussed in the Examples, other starting materials and reagents can be substituted to provide a variety of derivatives and/or reaction conditions. In addition, many of the exemplary compounds prepared by the described methods can be further modified in light of this disclosure using conventional chemistry.

In preparing compounds or a stereoisomer, atropisomer, tautomer, or pharmaceutically acceptable salt thereof as described herein protection of remote functionality (e.g., primary or secondary amine) of intermediates can be necessary. The need for such protection will vary depending on the nature of the remote functionality and the conditions of the preparation methods. Suitable amino-protecting groups include acetyl, trifluoroacetyl, t-butoxycarbonyl (BOC), benzyloxycarbonyl (CBz) and 9-fluorenylmethyleneoxycarbonyl (Fmoc). The need for such protection can be readily determined. For a general description of protecting groups and their use, see T. W. Greene, Protective Groups in Organic Synthesis, John Wiley & Sons, New York, 1991.

In the methods of preparing compounds or a stereoisomer, atropisomer, tautomer, or pharmaceutically acceptable salt thereof as described herein, it can be advantageous to separate reaction products from one another and/or from starting materials. The desired products of each step or series of steps is separated and/or purified to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (SMB) and preparative thin or thick layer chromatography, as well as techniques of small scale thin layer and flash chromatography.

Another class of separation methods involves treatment of a mixture with a reagent selected to bind to or render otherwise separable a desired product, unreacted starting material, reaction by product, or the like. Such reagents include adsorbents or absorbents such as activated carbon, molecular sieves, ion exchange media, or the like. Alternatively, the reagents can be acids in the case of a basic material, bases in the case of an acidic material, binding reagents such as antibodies, binding proteins, selective chelators such as crown ethers, liquid/liquid ion extraction reagents (LIX), or the like. Selection of appropriate methods of separation depends on the nature of the materials involved, such as, boiling point and molecular weight in distillation and sublimation, presence or absence of polar functional groups in chromatography, stability of materials in acidic and basic media in multiphase extraction, and the like.

Diastereomeric mixtures can be separated into their individual diastereomers on the basis of their physical chemical differences by methods such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Also, some of the compounds or a stereoisomer, atropisomer, tautomer, or pharmaceutically acceptable salt thereof as described herein described herein can be atropisomers (e.g., substituted biaryls). Enantiomers can also be separated by use of a chiral HPLC column.

A single stereoisomer, e.g., an enantiomer, substantially free of its stereoisomer can be obtained by resolution of the racemic mixture using a method such as formation of diastereomers using optically active resolving agents (Eliel, E. and Wilen, S. “Stereochemistry of Organic Compounds,” John Wiley & Sons, Inc., New York, 1994: Lochmuller, C. H., (1975) J. Chromatogr., 113(3):283-302). Racemic mixtures of chiral compounds or pharmaceutically acceptable salts thereof described herein can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: “Drug Stereochemistry, Analytical Methods and Pharmacology,” Irving W. Wainer, Ed., Marcel Dekker, Inc., New York (1993).

Under method (1), diastereomeric salts can be formed by reaction of enantiomerically pure chiral bases such as brucine, quinine, ephedrine, strychnine, α-methyl-β-phenylethylamine (amphetamine), and the like with asymmetric compounds bearing acidic functionality, such as carboxylic acid and sulfonic acid. The diastereomeric salts can be induced to separate by fractional crystallization or ionic chromatography. For separation of the optical isomers of amino compounds, addition of chiral carboxylic or sulfonic acids, such as camphorsulfonic acid, tartaric acid, mandelic acid, or lactic acid can result in formation of the diastereomeric salts.

Alternatively, by method (2), the substrate to be resolved is reacted with one enantiomer of a chiral compound to form a diastereomeric pair (E. and Wilen, S. “Stereochemistry of Organic Compounds”, John Wiley & Sons, Inc., 1994, p. 322). Diastereomeric compounds can be formed by reacting asymmetric compounds with enantiomerically pure chiral derivatizing reagents, such as menthyl derivatives, followed by separation of the diastereomers and hydrolysis to yield the pure or enriched enantiomer. A method of determining optical purity involves making chiral esters, such as a menthyl ester, e.g., (−) menthyl chloroformate in the presence of base, or Mosher ester, α-methoxy-α-(trifluoromethyl)phenyl acetate (Jacob III. J. Org. Chem. (1982) 47:4165), of the racemic mixture, and analyzing the ¹H NMR spectrum for the presence of the two atropisomeric enantiomers or diastereomers. Stable diastereomers of atropisomeric compounds can be separated and isolated by normal- and reverse-phase chromatography following methods for separation of atropisomeric naphthyl-isoquinolines (WO 96/15111). By method (3), a racemic mixture of two enantiomers can be separated by chromatography using a chiral stationary phase (“Chiral Liquid Chromatography” (1989) W. J. Lough, Ed., Chapman and Hall, New York; Okamoto, J. Chromatogr., (1990) 513:375-378). Enriched or purified enantiomers can be distinguished by methods used to distinguish other chiral molecules with asymmetric carbon atoms, such as optical rotation and circular dichroism.

The chemical reactions described herein may be readily adapted to prepare other compounds and pharmaceutically acceptable salts thereof described herein. For example, the synthesis of non-exemplified compounds and pharmaceutically acceptable salts thereof described herein may be successfully performed by modifications apparent to those skilled in the art, e.g., by appropriately protecting interfering groups, by utilizing other suitable reagents known in the art other than those described, or by making routine modifications of reaction conditions. Alternatively, other reactions disclosed herein or known in the art will be recognized as having applicability for preparing other compounds and pharmaceutically acceptable salts thereof described herein.

### Pharmaceutical Formulations

Also provided herein are pharmaceutical compositions comprising compound or a stereoisomer, atropisomer, tautomer, or pharmaceutically acceptable salt thereof as described herein and one or more pharmaceutically acceptable excipients.

Compounds or a stereoisomer, atropisomer, tautomer, or pharmaceutically acceptable salt thereof as described herein as described herein can be formulated in accordance with standard pharmaceutical practice as a pharmaceutical composition. Thus, further provided herein is a pharmaceutical composition comprising a compound or a stereoisomer, atropisomer, tautomer, or pharmaceutically acceptable salt thereof as described herein as described herein and one or more pharmaceutically acceptable excipients.

A typical formulation is prepared by mixing a compound or pharmaceutically acceptable salt thereof as described herein and an excipient. Suitable carriers, diluents and excipients include, but are not limited to, materials such as carbohydrates, waxes, water soluble and/or swellable polymers, hydrophilic or hydrophobic materials, gelatin, oils, solvents, water and the like. The particular excipient used will depend upon the means and purpose for which the compound or pharmaceutically acceptable salt thereof as described herein is being applied. Solvents are generally selected based on solvents recognized as safe (GRAS) to be administered to a mammal. In general, safe solvents are non-toxic aqueous solvents such as water and other non-toxic solvents that are soluble or miscible in water. Suitable aqueous solvents include water, ethanol, propylene glycol, polyethylene glycols (e.g., PEG 400, PEG 300), etc. and mixtures thereof. The formulations can also include one or more buffers, stabilizing agents, surfactants, wetting agents, lubricating agents, emulsifiers, suspending agents, preservatives, antioxidants, opaquing agents, glidants, processing aids, colorants, sweeteners, perfuming agents, flavoring agents and other known additives to provide an elegant presentation of the drug (i.e., a compound described herein or pharmaceutical composition thereof) or aid in the manufacturing of the pharmaceutical product (i.e., medicament).

The formulations can be prepared using conventional dissolution and mixing procedures. For example, the bulk drug substance (i.e., compound or pharmaceutically acceptable salt thereof as described herein or stabilized form thereof (e.g., complex with a cyclodextrin derivative or other known complexation agent) is dissolved in a suitable solvent in the presence of one or more of the excipients described above. The compound or a stereoisomer, atropisomer, tautomer, or pharmaceutically acceptable salt thereof as described herein as described herein is typically formulated into pharmaceutical dosage forms to provide an easily controllable dosage of the drug and to enable patient compliance with the prescribed regimen.

The pharmaceutical composition (or formulation) for application can be packaged in a variety of ways depending upon the method used for administering the drug. Generally, an article for distribution includes a container having deposited therein the pharmaceutical formulation in an appropriate form. Suitable containers include materials such as bottles (plastic and glass), sachets, ampoules, plastic bags, metal cylinders, and the like. The container can also include a tamper-proof assemblage to prevent indiscreet access to the contents of the package. In addition, the container has deposited thereon a label that describes the contents of the container. The label can also include appropriate warnings.

Pharmaceutical formulations of the compound or a stereoisomer, atropisomer, tautomer, or pharmaceutically acceptable salt thereof as described herein can be prepared for various routes and types of administration. For example, a compound or a stereoisomer, atropisomer, tautomer, or pharmaceutically acceptable salt thereof having the desired degree of purity can optionally be mixed with one or more pharmaceutically acceptable excipients (Remington's Pharmaceutical Sciences (1980) 16th edition, Osol, A. Ed.), in the form of a lyophilized formulation, milled powder, or an aqueous solution. Formulation can be conducted by mixing at ambient temperature at the appropriate pH, and at the desired degree of purity, with physiologically acceptable carriers, i.e., carriers that are non-toxic to recipients at the dosages and concentrations employed. The pH of the formulation depends mainly on the particular use and the concentration of compound, but can range from about 3 to about 8. For example, formulation in an acetate buffer at pH 5 can be a suitable embodiment.

The pharmaceutical composition ordinarily can be stored as a solid composition, a lyophilized formulation or as an aqueous solution.

The pharmaceutical compositions described herein can be formulated, dosed and administered in a fashion, i.e., amounts, concentrations, schedules, course, vehicles and route of administration, consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The effective amount of the compound or a stereoisomer, atropisomer, tautomer, or pharmaceutically acceptable salt thereof to be administered will be governed by such considerations, and is the minimum amount necessary to ameliorate, or treat the hyperproliferative disorder.

As a general proposition, the initial pharmaceutically effective amount of the compound or a stereoisomer, atropisomer, tautomer, or pharmaceutically acceptable salt thereof administered parenterally per dose will be in the range of about 0.01-100 mg/kg, namely about 0.1 to 20 mg/kg of patient body weight per day, with the typical initial range of compound used being 0.3 to 15 mg/kg/day. In another embodiment, a pharmaceutical composition described herein comprises an effective amount of a compound or a stereoisomer, atropisomer, tautomer, or pharmaceutically acceptable salt thereof described herein in an amount of about: 1 mg-10 mg; 10 mg-25 mg; 20 mg-50 mg; 50 mg-75 mg; 70 mg-100 mg; 100 mg-150 mg; 100 mg-200 mg; 100 mg-500 mg; 200 mg-500 mg; 250 mg-500 mg; 500 mg-1000 mg; or 750 mg-1000 mg.

Acceptable pharmaceutically acceptable excipients are nontoxic to recipients at the dosages and concentrations employed, and include buffers such as phosphate, citrate and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides and other carbohydrates including glucose, mannose, or dextrins; chelating agents such as EDTA; sugars such as sucrose, mannitol, trehalose or sorbitol; salt-forming counter-ions such as sodium: metal complexes (e.g., Zn-protein complexes); and/or non-ionic surfactants such as TWEEN™, PLURONICS™ or polyethylene glycol (PEG). The active pharmaceutical ingredients can also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations of compounds or pharmaceutically acceptable salts thereof as described herein may be prepared. Suitable examples of sustained-release preparations include semipermeable matrices of solid hydrophobic polymers containing a compound or pharmaceutically acceptable salt thereof as described herein, which matrices are in the form of shaped articles, e.g., films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and gamma-ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate) and poly-D-(−)-3-hydroxybutyric acid.

The formulations include those suitable for the administration routes detailed herein. The formulations can conveniently be presented in unit dosage form and can be prepared by any methods. Techniques and formulations generally are found in Remington's Pharmaceutical Sciences (Mack Publishing Co., Easton, PA). Such methods include the step of bringing into association the active ingredient with the carrier which constitutes one or more accessory ingredients. In general the formulations are prepared by uniformly and intimately bringing into association the active ingredient with liquid carriers or finely divided solid carriers or both, and then, if necessary, shaping the product.

Formulations of a compound or a stereoisomer, atropisomer, tautomer, or pharmaceutically acceptable salt thereof as described herein suitable for oral administration can be prepared as discrete units such as pills, capsules, cachets or tablets each containing a predetermined amount of such compound or a stereoisomer, atropisomer, tautomer, or pharmaceutically acceptable salt thereof. Compressed tablets can be prepared by compressing in a suitable machine the active ingredient in a free-flowing form such as a powder or granules, optionally mixed with a binder, lubricant, inert diluent, preservative, surface active or dispersing agent. Molded tablets can be made by molding in a suitable machine a mixture of the powdered active ingredient moistened with an inert liquid diluent. The tablets can optionally be coated or scored and optionally are formulated so as to provide slow or controlled release of the active ingredient therefrom. Tablets, troches, lozenges, aqueous or oil suspensions, dispersible powders or granules, emulsions, hard or soft capsules, e.g., gelatin capsules, syrups or elixirs can be prepared for oral use. Formulations of compounds or pharmaceutically acceptable salts thereof as described herein intended for oral use can be prepared according to any method for the manufacture of pharmaceutical compositions and such compositions can contain one or more agents including sweetening agents, flavoring agents, coloring agents and preserving agents, in order to provide a palatable preparation. Tablets containing the active ingredient in admixture with non-toxic pharmaceutically acceptable excipient which are suitable for manufacture of tablets are acceptable. These excipients can be, for example, inert diluents, such as calcium or sodium carbonate, lactose, calcium or sodium phosphate; granulating and disintegrating agents, such as maize starch, or alginic acid; binding agents, such as starch, gelatin or acacia; and lubricating agents, such as magnesium stearate, stearic acid or talc. Tablets can be uncoated or can be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax can be employed.

For treatment of the eye or other external tissues, e.g., mouth and skin, the formulations are preferably applied as a topical ointment or cream containing the active ingredient(s) in an amount of, for example, 0.075 to 20% w/w. When formulated in an ointment, the active ingredients can be employed with either a paraffinic or a water-miscible ointment base. Alternatively, the active ingredients can be formulated in a cream with an oil-in-water cream base. If desired, the aqueous phase of the cream base can include a polyhydric alcohol, i.e., an alcohol having two or more hydroxyl groups such as propylene glycol, butane 1,3-diol, mannitol, sorbitol, glycerol and polyethylene glycol (including PEG 400) and mixtures thereof. The topical formulations can desirably include a compound which enhances absorption or penetration of the active ingredient through the skin or other affected areas. Examples of such dermal penetration enhancers include dimethyl sulfoxide and related analogs. The oily phase of the emulsions of compositions provided herein can be constituted from known ingredients in a known manner. While the phase can comprise merely an emulsifier, it desirably comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and fat make up the so-called emulsifying ointment base which forms the oily dispersed phase of the cream formulations. Emulsifiers and emulsion stabilizers suitable for use in the formulation of described herein include Tween®60, Span®80, cetostearyl alcohol, benzyl alcohol, myristyl alcohol, glyceryl mono-stearate and sodium lauryl sulfate.

Aqueous suspensions comprising a compound or a stereoisomer, atropisomer, tautomer, or pharmaceutically acceptable salt thereof as described herein can contain the active materials in admixture with excipients suitable for the manufacture of aqueous suspensions. Such excipients include a suspending agent, such as sodium carboxymethylcellulose, croscarmellose, povidone, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension can also contain one or more preservatives such as ethyl or n-propyl p-hydroxybenzoate, one or more coloring agents, one or more flavoring agents and one or more sweetening agents, such as sucrose or saccharin.

The pharmaceutical compositions of a compound or a stereoisomer, atropisomer, tautomer, or pharmaceutically acceptable salt thereof as described herein can be in the form of a sterile injectable preparation, such as a sterile injectable aqueous or oleaginous suspension. This suspension can be formulated using suitable dispersing or wetting agents and suspending agents which have been mentioned above. The sterile injectable preparation can also be a sterile injectable solution or suspension in a non-toxic parenterally acceptable diluent or solvent, such as a solution in 1,3-butanediol or prepared as a lyophilized powder. Among the acceptable vehicles and solvents that can be employed are water, Ringers solution and isotonic sodium chloride solution. In addition, sterile fixed oils can conventionally be employed as a solvent or suspending medium. For this purpose any bland fixed oil can be employed including synthetic mono- or diglycerides. In addition, fatty acids such as oleic acid can likewise be used in the preparation of injectables.

The amount of active ingredient that can be combined with the carrier material to produce a single dosage form will vary depending upon the host treated and the particular mode of administration. For example, a time-release formulation intended for oral administration to humans can contain approximately 1 to 1000 mg of active material compounded with an appropriate and convenient amount of carrier material which can vary from about 5 to about 95% of the total compositions (weight:weight). The pharmaceutical composition can be prepared to provide easily measurable amounts for administration. For example, an aqueous solution intended for intravenous infusion can contain from about 3 to 500 μg of the active ingredient per milliliter of solution in order that infusion of a suitable volume at a rate of about 30 mL/hr can occur.

Formulations suitable for parenteral administration include aqueous and non-aqueous sterile injection solutions which can contain anti-oxidants, buffers, bacteriostats and solutes which render the formulation isotonic with the blood of the intended recipient; and aqueous and non-aqueous sterile suspensions which can include suspending agents and thickening agents.

Formulations suitable for topical administration to the eye also include eye drops wherein the active ingredient is dissolved or suspended in a suitable carrier, especially an aqueous solvent for the active ingredient. The active ingredient is preferably present in such formulations in a concentration of about 0.5 to 20% w/w, for example about 0.5 to 10% w/w, for example about 1.5% w/w.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier.

Formulations for rectal administration can be presented as a suppository with a suitable base comprising for example cocoa butter or a salicylate.

Formulations suitable for intrapulmonary or nasal administration have a particle size for example in the range of 0.1 to 500 microns (including particle sizes in a range between 0.1 and 500 microns in increments microns such as 0.5, 1, 30 microns, 35 microns, etc.), which is administered by rapid inhalation through the nasal passage or by inhalation through the mouth so as to reach the alveolar sacs. Suitable formulations include aqueous or oily solutions of the active ingredient. Formulations suitable for aerosol or dry powder administration can be prepared according to conventional methods and can be delivered with other therapeutic agents such as compounds heretofore used in the treatment or prophylaxis disorders as described below.

Formulations suitable for vaginal administration can be presented as pessaries, tampons, creams, gels, pastes, foams or spray formulations containing in addition to the active ingredient such carriers considered to be appropriate.

The formulations can be packaged in unit-dose or multi-dose containers, for example sealed ampoules and vials, and can be stored in a freeze-dried (lyophilized) condition requiring only the addition of the sterile liquid carrier, for example water, for injection immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

In one embodiment, the compound or a stereoisomer, atropisomer, tautomer, or pharmaceutically acceptable salt thereof are formulated as a prodrug. The term prodrug as used herein refers to a derivative of a compound that can be hydrolyzed, oxidized, or cleaved under biological conditions to provide the compound or a stereoisomer, atropisomer, tautomer, or pharmaceutically acceptable salt thereof. A prodrug as defined herein includes derivatives comprising one or more moieties that modulate or improve one or more physical, physiological or pharmaceutical property such as, but not limited to, solubility, permeability, uptake, biodistribution, metabolic stability, onset of action or some other druglike property, and is transformed to the bioactive or more biologically active substance as provided herein. In one embodiment, a prodrug herein has no biological activity until release of the compound or pharmaceutically acceptable salt thereof.

### Methods of Administration

Compounds or a stereoisomer, atropisomer, tautomer, or pharmaceutically acceptable salt thereof described herein can be administered by any route appropriate to the condition to be treated. Suitable routes include oral, parenteral (including subcutaneous, intramuscular, intravenous, intraarterial, intradermal, intrathecal and epidural), transdermal, rectal, nasal, topical (including buccal and sublingual), vaginal, intraperitoneal, intrapulmonary and intranasal. For local immunosuppressive treatment, the compounds can be administered by intralesional administration, including perfusing or otherwise contacting the graft with the inhibitor before transplantation. It will be appreciated that the preferred route can vary with for example the condition of the recipient. Where the compound or a stereoisomer, atropisomer, tautomer, or pharmaceutically acceptable salt thereof is administered orally, it can be formulated as a pill, capsule, tablet, etc. with a pharmaceutically acceptable carrier or excipient. Where the compound or a stereoisomer, atropisomer, tautomer, or pharmaceutically acceptable salt thereof is administered parenterally, it can be formulated with a pharmaceutically acceptable parenteral vehicle and in a unit dosage injectable form, as detailed below.

Thus, in one aspect provided herein is a pharmaceutical composition comprising a compound or pharmaceutically acceptable salt thereof as described herein and one or more pharmaceutically acceptable excipients. In one embodiment, compounds or a stereoisomer, atropisomer, tautomer, or pharmaceutically acceptable salt thereof described herein are administered as pharmaceutical compositions capable of being administered to a subject orally or parenterally. The compounds or a stereoisomer, atropisomer, tautomer, or pharmaceutically acceptable salt thereof described herein can be formulated for topical or parenteral use where the compound or a stereoisomer, atropisomer, tautomer, or pharmaceutically acceptable salt thereof is dissolved or otherwise suspended in a solution suitable for injections, suspensions, syrups, creams, ointments, gels, sprays, solutions and emulsions.

Oral administration can promote patient compliance in taking the compound (e.g. formulated as a pharmaceutical composition), thereby increasing compliance and efficacy. Oral pharmaceutical compositions comprising a compound described herein include, but are not limited to, tablets (e.g. coated, non-coated and chewable) and capsules (e.g. hard gelatin capsules, soft gelatin capsules, enteric coated capsules, and sustained release capsules). Tablets can be prepared by direct compression, by wet granulation, or by dry granulation. Oral pharmaceutical compositions comprising a compound described herein can be formulated for delayed or prolonged release.

A dose to treat human patients can range from about 10 mg to about 1000 mg of a compound described herein. A typical dose can be about 100 mg to about 300 mg of the compound. A dose can be administered once a day (QID), twice per day (BID), or more frequently, depending on the pharmacokinetic and pharmacodynamic properties, including absorption, distribution, metabolism, and excretion of the particular compound. Administration as used herein refers to the frequency of dosing and not, for example, the number of individual units a patient described herein must take for a dose. Thus, in some embodiments, a patient may take two or more dosage units (e.g. two or more pills/tablets/capsules) QD. In addition, toxicity factors can influence the dosage and administration regimen. When administered orally, the pill, capsule, or tablet can be ingested daily or less frequently for a specified period of time. The regimen can be repeated for a number of cycles of therapy.

### Methods of Treating and Uses

The compounds or a stereoisomer, atropisomer, tautomer, or pharmaceutically acceptable salt thereof described herein are useful as Ras inhibitors. In one aspect, the compounds or a stereoisomer, atropisomer, tautomer, or pharmaceutically acceptable salt thereof described herein are useful as KRas inhibitors. In another embodiment, the compounds or a stereoisomer, atropisomer, tautomer, or pharmaceutically acceptable salt thereof described herein are useful as KRasG12V inhibitors. In still another embodiment, the compounds or a stereoisomer, atropisomer, tautomer, or pharmaceutically acceptable salt thereof described herein are useful as pan-KRas inhibitors (i.e. compounds that inhibit the activity of a mutant KRas protein). In one embodiment, the compounds of Table 2 or a stereoisomer, atropisomer, tautomer, or pharmaceutically acceptable salt thereof described herein are useful as KRasG12D inhibitors. In such embodiments, such compounds are useful in the methods described herein where such cancer or disease is mediated by KRasG12D.

Provided herein are methods of contacting a cell, such as an ex vivo cell, with a compound or a stereoisomer, atropisomer, tautomer, or pharmaceutically acceptable salt thereof described herein, to inhibit KRas activity in the cell. In another embodiment, the activity is mutant KRasG12V activity. In another embodiment, the activity is mutant KRas activity (e.g. mutant pan-KRas activity).

As used herein, inhibition of the activity of more than one KRas mutant is referred to as pan-KRas inhibition. In such instances, a compound or pharmaceutically acceptable salt thereof as described herein inhibits the activity of more than one mutant KRas protein.

In certain instances, such compounds or pharmaceutically acceptable salts thereof selectively inhibit more than one mutant KRas protein relative to the wildtype (WT) KRas protein activity. In one such embodiment, a pan-KRas inhibitor as described herein and used in the methods provided herein inhibits more than one mutant KRas protein at least 5×, 8×, 10×, 12×, 15×, 20×, 24×, 27×, 50×, 100×, 500×, 700×, 1000×, 1300×, 1700×, 2000×, 5000×, or more greater than WT KRas protein. In one embodiment, such a KRas mutation is in the SWII domain. In one embodiment, such a KRas mutation corresponds to a change in the natural amino acid at the position corresponding to G12, G13, Q61, or A146. In some embodiments, the mutation corresponds to G12A, G12C, G12D, G12R, G12S, G12V, G13A, G13C, G13D, G13R, G13S, G13V, Q61E, Q61H, Q61K, Q61L, Q61P, Q61R, A146T, A146P, A146V, or A146T.

Further provided herein are methods of treating a cancer comprising a KRas mutation, the method comprising administering to a patient having such cancer, an effective amount of a compound or a stereoisomer, atropisomer, tautomer, or pharmaceutically acceptable salt thereof or a pharmaceutical composition as described herein. In one embodiment, the KRas mutation is a KRas^{G12V} mutation. In still another embodiment, the mutation is a known KRas mutation (e.g. treating with a compound or a stereoisomer, atropisomer, tautomer, or pharmaceutically acceptable salt thereof or a pharmaceutical composition as described herein that demonstrates pan-KRas inhibition).

In one embodiment, the methods further comprise testing a sample (e.g. as set forth herein) from the patient before administration of a compound of pharmaceutically acceptable salt thereof described herein for the absence or presence of a KRas^{G12V} mutation. In one such embodiment, a compound or stereoisomer, atropisomer, tautomer, or pharmaceutically acceptable salt thereof or pharmaceutical composition described herein is administered to the patient after the patient sample is determined to be positive for (e.g. the presence of) a KRas mutation. In one embodiment, the methods further comprise testing a sample (e.g. as set forth herein) from the patient before administration of a compound of pharmaceutically acceptable salt thereof described herein for the absence or presence of a KRas mutation, wherein the compound or stereoisomer, atropisomer, tautomer, or pharmaceutically acceptable salt thereof or pharmaceutical composition described herein is administered to the patient after the patient sample is determined to be positive for (e.g. the presence of) such KRas mutation.

The methods of treating a cancer described herein relate to the treatment of cancer such as acute myeloid leukemia, cancer in adolescents, childhood adrenocortical carcinoma, AIDS-related cancers (e.g. lymphoma and Kaposi's sarcoma), anal cancer, appendix cancer, astrocytomas, atypical teratoid rhabdoid tumor, basal cell carcinoma, bile duct cancer, bladder cancer, bone cancer, brain stem glioma, brain tumor, breast cancer, bronchial tumors, Burkitt lymphoma, carcinoid tumor, embryonal tumors, germ cell tumor, primary lymphoma, cervical cancer, childhood cancers, chordoma, cardiac tumors, chronic lymphocytic leukemia (CLL), chronic myelogenous leukemia (CML), chronic myeloproliferative disorders, colon cancer, colorectal cancer, craniopharyngioma, cutaneous T-cell lymphoma, extrahepatic ductal carcinoma in situ (DCIS), embryonal tumors, CNS cancer, endometrial cancer, ependymoma, esophageal cancer, esthesioneuroblastoma, Ewing sarcoma, extracranial germ cell tumor, extragonadal germ cell tumor, eye cancer, fibrous histiocytoma of bone, gall bladder cancer, gastric cancer, gastrointestinal carcinoid tumor, gastrointestinal stromal tumors (GIST), germ cell tumor, gestational trophoblastic tumor, hairy cell leukemia, head and neck cancer, heart cancer, liver cancer, Hodgkin's lymphoma, hypopharyngeal cancer, intraocular melanoma, islet cell tumors, pancreatic neuroendocrine tumors, kidney cancer, laryngeal cancer, lip and oral cavity cancer, lobular carcinoma in situ (LCIS), lung cancer, lymphoma, metastatic squamous neck cancer with occult primary, midline tract carcinoma, mouth cancer, multiple endocrine neoplasia syndromes, multiple myeloma/plasma cell neoplasm, mycosis fungoides, myelodysplastic syndromes, myelodysplastic/myeloproliferative neoplasms, multiple myeloma, Merkel cell carcinoma, malignant mesothelioma, malignant fibrous histiocytoma of bone and osteosarcoma, nasal cavity and paranasal sinus cancer, nasopharyngeal cancer, neuroblastoma, non-Hodgkin's lymphoma, non-small cell lung cancer (NSCLC), oral cancer, oropharyngeal cancer, ovarian cancer, pancreatic cancer, papillomatosis, paraganglioma, parathyroid cancer, penile cancer, pharyngeal cancer, pleuropulmonary blastoma, primary central nervous system (CNS) lymphoma, prostate cancer, rectal cancer, transitional cell cancer, retinoblastoma, rhabdomyosarcoma, salivary gland cancer, skin cancer, small cell lung cancer, small intestine cancer, soft tissue sarcoma, T-Cell lymphoma, testicular cancer, throat cancer, thymoma and thymic carcinoma, thyroid cancer, transitional cell cancer of the renal pelvis and ureter, trophoblastic tumor, unusual cancers of childhood, urethral cancer, uterine sarcoma, vaginal cancer, vulvar cancer, or viral-induced cancer.

In some embodiments, the cancer is a hematological cancer, pancreatic cancer, MYH associated polyposis, colorectal cancer or lung cancer. In one embodiment, the cancer is lung cancer, colorectal cancer, appendiceal cancer, or pancreatic cancer. In one embodiment, the cancer is pancreatic cancer, lung cancer, or colon cancer. The lung cancer can be adenocarcinoma, non-small cell lung cancer (NSCLC), or small cell lung cancer (SCLC). In one embodiment, the cancer is colorectal cancer. In another embodiment, the cancer is pancreatic cancer. In one embodiment, the cancer is lung adenocarcinoma.

The methods provided herein can also comprise testing a sample from the patient before administration of a compound or stereoisomer, atropisomer, tautomer, or pharmaceutically acceptable salt thereof described herein for the absence or presence of a KRas mutation corresponding to the 12 position of KRas (e.g. Gly12). In one embodiment, a compound, stereoisomer, atropisomer, tautomer, or pharmaceutically acceptable salt thereof or pharmaceutical composition is administered to the patient after the patient sample shows the presence of a KRas mutation corresponding to the 12 position of KRas (e.g. Gly12). In one embodiment, a compound or stereoisomer, atropisomer, tautomer, or pharmaceutically acceptable salt thereof described herein is not administered unless a patient sample comprises a KRas mutation corresponding to the 12 position of KRas (e.g. Gly12).

The methods provided herein can also comprise testing a sample from the patient before administration of a compound or stereoisomer, atropisomer, tautomer, or pharmaceutically acceptable salt thereof described herein for the absence or presence of a KRas^{G12V} mutation. In one embodiment, a compound, stereoisomer, atropisomer, tautomer, or pharmaceutically acceptable salt thereof or pharmaceutical composition is administered to the patient after the patient sample shows the presence of a KRas^{G12V} mutation. In one embodiment, a compound or stereoisomer, atropisomer, tautomer, or pharmaceutically acceptable salt thereof described herein is not administered unless a patient sample comprises a KRas^{G12V} mutation.

The methods provided herein can further comprise testing a sample from the patient before administration of a compound or stereoisomer, atropisomer, tautomer, or pharmaceutically acceptable salt thereof described herein for the absence or presence of a KRas mutation, where the compound or stereoisomer, atropisomer, tautomer, or pharmaceutically acceptable salt thereof described herein has pan-KRas inhibition. In one embodiment, a compound, stereoisomer, atropisomer, tautomer, or pharmaceutically acceptable salt thereof or pharmaceutical composition is administered to the patient after the patient sample shows the presence of a KRas mutation, where the compound or stereoisomer, atropisomer, tautomer, or pharmaceutically acceptable salt thereof described herein has pan-KRas inhibition. In one embodiment, a compound or stereoisomer, atropisomer, tautomer, or pharmaceutically acceptable salt thereof described herein is not administered unless a patient sample comprises a KRas mutation, where the compound or stereoisomer, atropisomer, tautomer, or pharmaceutically acceptable salt thereof described herein has pan-KRas inhibition.

In one embodiment, the cancer is pancreatic cancer, lung cancer, or colorectal cancer. In another embodiment, the pancreatic cancer, lung cancer, or colorectal cancer comprises a KRas^{G12V} mutation. In still another embodiment, the cancer is tissue agnostic but comprises a KRas^{G12V} mutation.

In another embodiment, the pancreatic cancer, lung cancer, or colorectal cancer comprises a KRas mutation. In one such embodiment, the cancer is tissue agnostic but comprises a KRas mutation. In such embodiments, the cancer can be treated as described herein with a compound or stereoisomer, atropisomer, tautomer, or pharmaceutically acceptable salt thereof described herein having pan-KRas inhibition.

Further provided herein are methods of treating lung cancer comprising a KRas^{G12V} mutation in a patient having such a lung cancer, the method comprising administering to the patient an effective amount of a compound or a stereoisomer, atropisomer, tautomer, or pharmaceutically acceptable salt thereof (or a pharmaceutical composition comprising the same) described herein to the patient. In one embodiment, the lung cancer is non-small cell lung carcinoma (NSCLC). The NSCLC can be, for example, adenocarcinoma, squamous-cell lung carcinoma or large-cell lung carcinoma.

In another embodiment, the lung cancer is small cell lung carcinoma. In still another embodiment, the lung cancer is glandular tumors, carcinoid tumors or undifferentiated carcinomas. The lung cancer can be stage I or II lung cancer. In one embodiment, the lung cancer is stage III or IV lung cancer. The methods provided herein include administration of the compound as a 1L therapy.

Still further provided herein are methods of treating lung cancer comprising a KRas mutation (e.g. corresponding to position Gly12) in a patient having such a lung cancer, the method comprising administering to the patient an effective amount of a compound or a stereoisomer, atropisomer, tautomer, or pharmaceutically acceptable salt thereof (or a pharmaceutical composition comprising the same) described herein, where the compound or stereoisomer, atropisomer, tautomer, or pharmaceutically acceptable salt thereof described herein has pan-KRas inhibition, to the patient. In one embodiment, the lung cancer is non-small cell lung carcinoma (NSCLC). The NSCLC can be, for example, adenocarcinoma, squamous-cell lung carcinoma or large-cell lung carcinoma.

In another embodiment, the lung cancer is small cell lung carcinoma. In still another embodiment, the lung cancer is glandular tumors, carcinoid tumors or undifferentiated carcinomas. The lung cancer can be stage I or II lung cancer. In one embodiment, the lung cancer is stage III or IV lung cancer. The methods provided herein include administration of the compound as a 1L therapy.

Further provided herein are methods of treating pancreatic cancer comprising a KRas^{G12V} mutation in a patient having such pancreatic cancer, the method comprising administering to the patient an effective amount of a compound or stereoisomer, atropisomer, tautomer, or pharmaceutically acceptable salt thereof described herein to the patient. In one embodiment, the patient has been previously treated with radiation and one or more chemotherapy agents. In one embodiment, the pancreatic cancer is stage 0, I, or II. In another embodiment, the pancreatic cancer is stage III or stage IV.

Further provided herein are methods of treating pancreatic cancer comprising a KRas mutation (e.g. corresponding to position Gly12) in a patient having such pancreatic cancer, the method comprising administering to the patient an effective amount of a compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition, to the patient. In one embodiment, the patient has been previously treated with radiation and one or more chemotherapy agents. In one embodiment, the pancreatic cancer is stage 0, I, or II. In another embodiment, the pancreatic cancer is stage III or stage IV.

Still further provided herein are methods of treating colon cancer comprising a KRas^{G12V} mutation in a patient having such colon cancer, the method comprising administering to the patient an effective amount of a compound or stereoisomer, atropisomer, tautomer, or pharmaceutically acceptable salt thereof described herein to the patient. In one embodiment, the colon cancer is stage I or II. In another embodiment, the colon cancer is stage III or stage IV.

Still further provided herein are methods of treating colon cancer comprising a KRas mutation (e.g. corresponding to position Gly12) in a patient having such colon cancer, the method comprising administering to the patient an effective amount of a compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition, to the patient. In one embodiment, the colon cancer is stage I or II. In another embodiment, the colon cancer is stage III or stage IV.

Further provided herein are methods of treating tissue agnostic cancer comprising a KRas^{G12V} mutation. In one embodiment of such methods, the method (Ag2) comprises:
(a) determining the absence or presence of a KRas^{G12V} mutation in a sample taken from a patient with a suspected diagnosed cancer; and
(b) administering to the patient an effective amount of a compound or stereoisomer, atropisomer, tautomer, or pharmaceutically acceptable salt thereof described herein.

Further provided herein are methods of treating tissue agnostic cancer comprising a KRas mutation (e.g. corresponding to position Gly12). In one embodiment of such methods, the method (Ag3) comprises:
(a) determining the absence or presence of a KRas mutation in a sample taken from a patient with a suspected diagnosed cancer; and
(b) administering to the patient an effective amount of a compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition.

In one embodiment of the methods of Ag1, Ag2, and Ag3, the patient is diagnosed with a cancer described herein. In another embodiment of the methods of Ag1, Ag2, and Ag3, the sample is a tumor sample taken from the subject. In one such embodiment, the sample is taken before administration of any therapy. In another such embodiment, the sample is taken before administration of a compound or stereoisomer, atropisomer, tautomer, or pharmaceutically acceptable salt thereof described herein and after administration of another chemotherapeutic agent. In another embodiment of the methods of Ag1, Ag2, and Ag3, the compound or stereoisomer, atropisomer, tautomer, or pharmaceutically acceptable salt thereof described herein is administered as provided herein (e.g. orally).

Also provided herein is a compound or stereoisomer, atropisomer, tautomer, or pharmaceutically acceptable salt thereof for use as a therapeutically active substance. In another such embodiment, the compound or stereoisomer, atropisomer, tautomer, or pharmaceutically acceptable salt thereof can be for the therapeutic treatment of a cancer comprising a KRas^{G12V} mutation. In still another such embodiment, the compound or stereoisomer, atropisomer, tautomer, or pharmaceutically acceptable salt thereof can be for the therapeutic treatment of a cancer comprising a KRas mutation (e.g. corresponding to position Gly12) where the compound or stereoisomer, atropisomer, tautomer, or pharmaceutically acceptable salt thereof described herein has pan-KRas inhibition.

Further provided herein a compound or stereoisomer, atropisomer, tautomer, or pharmaceutically acceptable salt thereof for the therapeutic and/or prophylactic treatment of a cancer comprising a KRas^{G12V} mutation. Still further provided herein is a compound or stereoisomer, atropisomer, tautomer, or pharmaceutically acceptable salt thereof for the therapeutic and/or prophylactic treatment of a cancer comprising a KRas mutation (e.g. corresponding to position Gly12) where the compound or stereoisomer, atropisomer, tautomer, or pharmaceutically acceptable salt thereof described herein has pan-KRas inhibition.

In one embodiment, a compound or stereoisomer, atropisomer, tautomer, or pharmaceutically acceptable salt thereof described herein is used in the preparation of a medicament for the therapeutic treatment of a cancer comprising a KRas^{G12V} mutation. In one embodiment, a compound or stereoisomer, atropisomer, tautomer, or pharmaceutically acceptable salt thereof described herein is used in the preparation of a medicament for the therapeutic treatment of a cancer comprising a KRas mutation (e.g. corresponding to position Gly12) where the compound or stereoisomer, atropisomer, tautomer, or pharmaceutically acceptable salt thereof described herein has pan-KRas inhibition.

Still further provided herein are uses of a compound or stereoisomer, atropisomer, tautomer, or pharmaceutically acceptable salt thereof as described herein in the manufacture of a medicament for inhibiting tumor metastasis.

Further provided herein are methods for inhibiting tumor metastasis, the method comprising administering to a patient having a tumor a therapeutically effective amount of a compound or stereoisomer, atropisomer, tautomer, or pharmaceutically acceptable salt thereof described herein. In one embodiment, the inhibition is of a tumor comprising a KRas^{G12V} mutation. In one embodiment, the inhibition is of a tumor comprising a KRas mutation (e.g. corresponding to position Gly12) where the compound or stereoisomer, atropisomer, tautomer, or pharmaceutically acceptable salt thereof described herein has pan-KRas inhibition. In another embodiment, inhibiting tumor metastasis in a patient described herein results in reduction of tumor size. In another embodiment, inhibiting tumor metastasis in a patient described herein results in stabilizing (e.g. no further growth) of tumor size. In another embodiment, inhibiting tumor metastasis in a patient described herein results in remission of the cancer and/or its symptoms.

Further provided herein are methods for inhibiting proliferation of a cell population, the method comprising contacting the cell population with a compound or stereoisomer, atropisomer, tautomer, or pharmaceutically acceptable salt thereof described herein. In one embodiment, the cell population is in a human patient. In another embodiment, the cell population comprises a KRas^{G12V} mutation. In another embodiment, the cell population comprises a KRas mutation (e.g. corresponding to position Gly12) where the compound or stereoisomer, atropisomer, tautomer, or pharmaceutically acceptable salt thereof described herein has pan-KRas inhibition.

Further provided herein are methods of inhibiting KRas in a patient in need of therapy, comprising administering to the patient a therapeutically effective amount of a compound or stereoisomer, atropisomer, tautomer, or pharmaceutically acceptable salt thereof described herein. In one embodiment, the KRas inhibited is KRas^{G12V}. In one embodiment, the KRas inhibited is a mutant KRas protein (e.g. corresponding to position Gly12) where the compound or stereoisomer, atropisomer, tautomer, or pharmaceutically acceptable salt thereof described herein has pan-KRas inhibition. In another embodiment, inhibiting KRas results in decreased tumor size. In another embodiment, inhibiting KRas results in remission of the cancer and/or its symptoms.

Further provided herein are methods for regulating activity of a KRas mutant protein, the method comprising reacting the mutant protein with a compound or stereoisomer, atropisomer, tautomer, or pharmaceutically acceptable salt thereof described herein. In one embodiment, the mutant protein comprises a KRas^{G12V} mutation. In one embodiment, the mutant protein comprises a KRas mutation where the compound or stereoisomer, atropisomer, tautomer, or pharmaceutically acceptable salt thereof described herein has pan-KRas inhibition. In one embodiment, the activity of KRas is decreased after contacting with a compound or stereoisomer, atropisomer, tautomer, or pharmaceutically acceptable salt thereof described herein. In another embodiment, the downregulation of activity of the KRas mutant protein treats a cancer described herein in a patient described herein. In another embodiment, the downregulation of activity of the KRas mutant protein results in decreased tumor size. In another embodiment, the downregulation of activity of the KRas mutant protein results in remission of a cancer described herein and/or its symptoms.

In some embodiments, the methods provided herein comprise inhibiting KRas^{G12V} activity in a cell by contacting said cell with an amount of a compound or stereoisomer, atropisomer, tautomer, or pharmaceutically acceptable salt thereof described herein sufficient to inhibit the activity of KRas^{G12V} in said cell. In some embodiments, the methods provided herein comprise inhibiting KRas^{G12V} activity in a tissue by contacting said tissue with an amount of a compound or stereoisomer, atropisomer, tautomer, or pharmaceutically acceptable salt thereof described herein sufficient to inhibit the activity of KRas^{G12V} in said tissue. In some embodiments, the methods provided herein comprise inhibiting KRas^{G12V} activity in a patient described herein by contacting said patient with an amount of a compound or stereoisomer, atropisomer, tautomer, or pharmaceutically acceptable salt thereof described herein sufficient to inhibit the activity of KRas^{G12V} in said patient.

In some embodiments, the methods provided herein comprise inhibiting mutant KRas (e.g. mutation at Gly12) activity in a cell by contacting said cell with an amount of a compound or stereoisomer, atropisomer, tautomer, or pharmaceutically acceptable salt thereof described herein sufficient to inhibit the activity of mutant KRas (e.g. mutation at Gly12) in said cell. In some embodiments, the methods provided herein comprise inhibiting mutant KRas (e.g. mutation at Gly12) activity in a tissue by contacting said tissue with an amount of a compound or stereoisomer, atropisomer, tautomer, or pharmaceutically acceptable salt thereof described herein sufficient to inhibit the activity of mutant KRas (e.g. mutation at Gly12) in said tissue. In some embodiments, the methods provided herein comprise inhibiting mutant KRas (e.g. mutation at Gly12) activity in a patient described herein by contacting said patient with an amount of a compound or stereoisomer, atropisomer, tautomer, or pharmaceutically acceptable salt thereof described herein sufficient to inhibit the activity of mutant KRas (e.g. mutation at Gly12) in said patient. In such embodiments, it is understood that the compound or stereoisomer, atropisomer, tautomer, or pharmaceutically acceptable salt thereof described herein has pan-KRas inhibition.

Further provided herein are methods for preparing a labeled KRas^{G12V} mutant protein, the method comprising reacting a KRas^{G12V} mutant protein with a labeled compound or stereoisomer, atropisomer, tautomer, or pharmaceutically acceptable salt thereof described herein to result in the labeled KRas^{G12V} mutant protein. In one embodiment, the label is an imaging agent. In one embodiment, the labeled KRas^{G12V} can be used to detect the absence or presence of KRas^{G12V} mutant protein in a patient sample, thereby detecting the presence or absence of a cancer mediated by mutant KRas.

Further provided herein are methods for preparing a labeled KRas mutant protein (e.g. mutation at Gly12), the method comprising reacting a KRas mutant protein with a labeled compound or stereoisomer, atropisomer, tautomer, or pharmaceutically acceptable salt thereof described herein, where the compound or stereoisomer, atropisomer, tautomer, or pharmaceutically acceptable salt thereof described herein has pan-KRas inhibition, to result in the labeled KRas mutant protein. In one embodiment, the label is an imaging agent. In one embodiment, the labeled mutant KRas protein can be used to detect the absence or presence of mutant KRas in a patient sample, thereby detecting the presence or absence of a cancer mediated by mutant KRas.

Still further provided herein are methods of inhibiting Ras-mediated cell signaling. In one embodiment, the methods comprise contacting a cell with an effective amount of one or more compounds or a stereoisomer, atropisomer, tautomer, or pharmaceutically acceptable salt thereof disclosed herein thereof. Inhibition of Ras-mediated signal transduction can be assessed and demonstrated by a wide variety of ways known in the art. Non-limiting examples include a showing of (a) a decrease in GTPase activity of Ras; (b) a decrease in GTP binding affinity or an increase in GDP binding affinity; (c) an increase in K off of GTP or a decrease in K off of GDP; (d) a decrease in the levels of signaling transduction molecules downstream in the Ras pathway, such as a decrease in pMEK level; and/or (e) a decrease in binding of Ras complex to downstream signaling molecules including but not limited to Raf. Kits and commercially available assays can be utilized for determining one or more of the above.

KRas mutations have also been identified in hematological malignancies (e.g., cancers that affect blood, bone marrow, and/or lymph nodes). Accordingly, certain embodiments are directed to administration of a disclosed compound or stereoisomer, atropisomer, tautomer, or pharmaceutically acceptable salt thereof (e.g., in the form of a pharmaceutical composition) as described herein to a patient in need of treatment of a hematological malignancy. Such malignancies include, but are not limited to leukemias and lymphomas. For example, the presently disclosed compounds can be used for treatment of diseases such as acute lymphoblastic leukemia (ALL), acute myelogenous leukemia (AML), chronic lymphocytic leukemia (CLL), small lymphocytic lymphoma (SLL), chronic myelogenous leukemia (CML), acute monocytic leukemia (AMoL) and/or other leukemias. In other embodiments, the compounds or a pharmaceutically acceptable salt thereof described herein are useful for treatment of lymphomas such as all subtypes of Hodgkin's lymphoma or non-Hodgkin's lymphoma.

Determining whether a tumor or cancer comprises a KRas mutation as described here can be undertaken by assessing the nucleotide sequence encoding the KRas protein, by assessing the amino acid sequence of the KRas protein, or by assessing the characteristics of a putative KRas mutant protein. The sequence of wild-type human KRas (e.g. Accession No. NP203524) is known in the art.

Methods for detecting a mutation in a KRas nucleotide sequence are known by those of skill in the art. These methods include, but are not limited to, polymerase chain reaction-restriction fragment length polymorphism (PCR-RFLP) assays, polymerase chain reaction-single strand conformation polymorphism (PCR-SSCP) assays, real-time PCR assays, PCR sequencing, mutant allele-specific PCR amplification (MASA) assays, direct sequencing, primer extension reactions, electrophoresis, oligonucleotide ligation assays, hybridization assays, TaqMan assays, SNP genotyping assays, high resolution melting assays and microarray analyses. In some embodiments, samples are evaluated for KRas mutations described herein by real-time PCR. In real-time PCR, fluorescent probes specific for the KRas mutation are used. When a mutation is present, the probe binds and fluorescence is detected. In some embodiments, the KRas mutation is identified using a direct sequencing method of specific regions (e.g., exon 2 and/or exon 3) in the KRas gene. This technique will identify all possible mutations in the region sequenced.

Methods for determining whether a tumor or cancer comprises a KRas mutation described herein can use a variety of samples. In some embodiments, the sample is taken from a subject having a tumor or cancer. In some embodiments, the sample is a fresh tumor/cancer sample. In some embodiments, the sample is a frozen tumor/cancer sample. In some embodiments, the sample is a formalin-fixed paraffin-embedded sample. In some embodiments, the sample is processed to a cell lysate. In some embodiments, the sample is processed to DNA or RNA.

Further provided herein are uses of a compound or stereoisomer, atropisomer, tautomer, or pharmaceutically acceptable salt thereof described herein, in the manufacture of a medicament for treating cancer. In some embodiments, the medicament is formulated for oral administration. In some embodiments, the medicament is formulated for injection. In some embodiments, the cancer comprises a KRas^{G12V} mutation. In some embodiments, the cancer comprises a KRas mutation (e.g. mutation at Gly12) where the compound or stereoisomer, atropisomer, tautomer, or pharmaceutically acceptable salt thereof described herein has pan-KRas inhibition. In some embodiments, the cancer is a hematological cancer, pancreatic cancer, MYH associated polyposis, colorectal cancer or lung cancer. In one embodiment, the cancer is lung cancer, colorectal cancer, or pancreatic cancer. In one embodiment, the cancer is colorectal cancer. In another embodiment, the cancer is pancreatic cancer. In some embodiments, the cancer is lung adenocarcinoma. In some embodiments, are uses of a compound or stereoisomer, atropisomer, tautomer, or pharmaceutically acceptable salt thereof described herein, in the manufacture of a medicament for inhibiting tumor metastasis.

Further provided herein is a compound or a pharmaceutically acceptable salt thereof described herein, for use in a method of treating cancer. In one embodiment, the cancer comprises a KRas^{G12V} mutation. In one embodiment, the cancer comprises a KRas mutation (e.g. mutation at Gly12) where the compound or stereoisomer, atropisomer, tautomer, or pharmaceutically acceptable salt thereof described herein has pan-KRas inhibition In one such embodiment, the cancer is a hematological cancer, pancreatic cancer, MYH associated polyposis, colorectal cancer or lung cancer. In one such embodiment, the cancer is lung cancer, colorectal cancer, or pancreatic cancer. In one such embodiment, the cancer is colorectal cancer. In one such embodiment, the cancer is pancreatic cancer. In one such embodiment, the cancer is lung adenocarcinoma.

### Combination Therapies

The compounds or a stereoisomer, atropisomer, tautomer, or pharmaceutically acceptable salt thereof described herein may be employed alone or in combination with other therapeutic agents for the treatment of a disease or disorder described herein. The second compound of the pharmaceutical combination formulation or dosing regimen preferably has complementary activities to the compound or a pharmaceutically acceptable salt thereof described herein such that they do not adversely affect each other. The combination therapy may provide “synergy” and prove “synergistic”, i.e., the effect achieved when the active ingredients used together is greater than the sum of the effects that results from using the compounds separately.

The combination therapy may be administered as a simultaneous or sequential regimen. When administered sequentially, the combination may be administered in two or more administrations. The combined administration includes co-administration, using separate formulations or a single pharmaceutical formulation, and consecutive administration in either order, wherein preferably there is a time period while both (or all) active agents simultaneously exert their biological activities.

Combination therapies herein comprise the administration of a compound or stereoisomer, atropisomer, tautomer, or pharmaceutically acceptable salt thereof described herein, and the use of at least one other treatment method. The amounts of the compound or stereoisomer, atropisomer, tautomer, or pharmaceutically acceptable salt thereof described herein and the other pharmaceutically active agent(s) and the relative timings of administration will be selected in order to achieve the desired combined therapeutic effect.

In various embodiments of the method, the additional therapeutic agent is an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor (such as irinotecan, or such as etoposide, or such as doxorubicin), a taxane (such as anti-microtubule agents including paclitaxel and docetaxel), an anti-metabolite agent (such as 5-FU or such as gemcitabine), or an alkylating agent (such as cisplatin or such as cyclophosphamide), or a taxane.

In some embodiments, the additional therapeutic agent is an epidermal growth factor receptor (EGFR) inhibitor, such as Erlotinib or such as Afatinib. In some embodiments the additional therapeutic agent is gefitinib, osimertinib, or dacomitinib. In some embodiments the additional therapeutic agent is a monoclonal antibody such as cetuximab (Erbitux) or panitumumab (Vectibix). In some embodiments the EGFR inhibitor is a dual or pan-HER inhibitor. In other embodiments, the additional therapeutic agent is a phosphatidylinositol-3-kinase (PI3K) inhibitor, such as GDC-0077, GDC-0941, MLN1117, BYL719 (Alpelisib) or BKM120 (Buparlisib). GDC-0941 refers to 2-(1H-indazol-4-yl)-6-(4-methanesulfonyl-piperazin-1-ylmethyl)-4-morpholin-4-yl-thieno[3,2-d]pyrimidine or a salt thereof (e.g., bismesylate salt).

In still other embodiments, the additional therapeutic agent is an insulin-like growth factor receptor (IGF1R) inhibitor. For example, in some embodiments the insulin-like growth factor receptor (IGF1R) inhibitor is NVP-AEW541. In other embodiments, the additional therapeutic agent is IGOSI-906 (Linsitinib), BMS-754807, or in other embodiments the additional therapeutic agent is a neutralizing monoclonal antibody specific to IGF1R such as AMG-479 (ganitumab), CP-751,871 (figitumumab), IMC-A12 (cixutumumab), MK-0646 (dalotuzumab), or R-1507 (robatumumab).

In some other embodiments, the additional therapeutic agent is a Janus kinase (JAK) inhibitor. In some embodiments, the additional therapeutic agent is CYT387, GLPG0634, Baricitinib, Lestaurtinib, momelotinib, Pacritinib, Ruxolitinib, or TG101348.

In some other embodiments, the additional therapeutic agent is an anti-glypican 3 antibody. In some embodiments, the anti-glypican 3 antibody is codrituzumab.

In some other embodiments, the additional therapeutic agent is an antibody drug conjugate (ADC). In some embodiments, the ADC is polatuzumab vedotin, RG7986, RG7882, RG6109, or RO7172369.

In some other embodiments, the additional therapeutic agent is an MDM2 antagonist. In some embodiments, the MDM2 antagonist is idasanutlin.

In some other embodiments, the additional therapeutic agent is an agonistic antibody against CD40. In some embodiments, the agonistic antibody against CD40 is selicrelumab (RG7876).

In some other embodiments, the additional therapeutic agent is a bispecific antibody. In some embodiments, the bispecific antibody is RG7828 (BTCT4465A), RG7802, RG7386 (FAP-DR5), RG6160, RG6026, ERY974, or anti-HER2/CD3.

In some other embodiments, the additional therapeutic agent is a targeted immunocytokine. In some embodiments, the targeted immunocytokine is RG7813 or RG7461.

In some other embodiments, the additional therapeutic agent is an antibody targeting colony stimulating factor-1 receptor (CSF-1R). In some embodiments, the CSF-1R antibody is emactuzumab.

In some other embodiments, the additional therapeutic agent is a personalised cancer vaccine. In some embodiments, the personalised cancer vaccine is RG6180.

In some other embodiments, the additional therapeutic agent is an inhibitor of BET (bromodomain and extraterminal family) proteins (BRD2/3/4/T). In some embodiments, the BET inhibitor is RG6146.

In some other embodiments, the additional therapeutic agent is an antibody designed to bind to TIGIT. In some embodiments, the anti-TIGIT antibody is RG6058 (MTIG7192A).

In some other embodiments, the additional therapeutic agent is a selective estrogen receptor degrader (SERD). In some other embodiments, the SERD is RG6047 (GDC-0927) or RG6171 (GDC-9545).

In some other embodiments the additional therapeutic agent is an MET kinase inhibitor, such as Crizotinib, tivantinib, AMG337, cabozantinib, or foretinib. In other embodiments the additional therapeutic agent is a neutralizing monoclonal antibody to MET such as onartuzumab.

In more embodiments, the additional therapeutic agent is a SRC family non-receptor tyrosine kinase inhibitor. For example, in some embodiments the additional therapeutic agent is an inhibitor of the subfamily of SRC family non-receptor tyrosine kinases. Exemplary inhibitors in this respect include Dasatinib. Other examples in this regard include Ponatinib, saracatinib, and bosutinib.

In yet other embodiments, the additional therapeutic agent is a mitogen-activated protein kinase (MEK) inhibitor. In some of these embodiments, the mitogen-activated protein kinase (MEK) inhibitor is trametinib, selumetinib, COTELLIC® (cobimetinib), PD0325901, or RO5126766. In other embodiments the MEK inhibitor is GSK-1120212, also known as trametinib.

In yet other embodiments, the additional therapeutic agent is an extracellular-signal-regulated kinase (ERK) inhibitor. In some of these embodiments, the mitogen-activated protein kinase (MEK) inhibitor is SCH722984 or GDC-0994.

In other embodiments the protein kinase inhibitor is taselisib, ipatasertib, GDC-0575, GDC-5573 (HM95573), RG6114 (GDC-0077), CK127, Afatinib, Axitinib, Atezolizumab, Bevacizumab, Bostutinib, Cetuximab, Crizotinib, Dasatinib, Erlotinib, Fostamatinib, Gefitinib, Imatinib, Lapatinib, Lenvatinib, Ibrutinib, Nilotinib, Panitumumab, Pazopanib, Pegaptanib, Ranibizumab, Ruxolitinib, Sorafenib, Sunitinib, SU6656, Trastuzumab, Tofacitinib, Vandetanib, or Vemurafenib. In still more embodiments, the additional therapeutic agent is a topoisomerase inhibitor. In some of these embodiments, the topoisomerase inhibitor is Irinotecan. In some more embodiments, the additional therapeutic agent is a taxane. Exemplary taxanes include Taxol and Docetaxel.

In addition to the above additional therapeutic agent, other chemotherapeutics are presently known in the art and can be used in combination with the compounds and pharmaceutically acceptable salts thereof described herein. In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adramycin as well as a host of chemotherapeutic agents. Non-limiting examples of chemotherapeutic agents include alkylating agents such as thiotepa and cyclosphosphamide (CYTOXAN™) alkyl sulfonates such as busulfan, improsulfan and piposulfan; aziridines such as benzodopa, carboquone, meturedopa, and uredopa; ethylenimines and methyl melamines including altretamine, triethylenemelamine, triethylenephosphoramide, triethylenethiophosphaoramide and trimethylol melamine; nitrogen mustards such as chlorambucil, chlomaphazine, cyclophosphamide, estramustine, ifosfamide, mechlorethamine, mechlorethamine oxide hydrochloride, melphalan, novembichin, phenesterine, prednimustine, trofosfamide, uracil mustard: nitrosoureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU): folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide K; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; Xeloda®; ibandronate; camptothecin-11 (CPT-11); topoisomerase inhibitor RFS 2000; and difluoromethylomithine (DMFO). Where desired, the compounds or pharmaceutical acceptable salts thereof or pharmaceutical composition as described herein can be used in combination with commonly prescribed anti-cancer drugs such as Herceptin®, Avastin®, Gazyva®, Tecentriq®, Alecensa®, Perjeta®, Venclexta™, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tanquidar, Tegafur-uracil, Temodar, Tesetaxel, Triplatin tetranitrate, Tris(2-chloroethyl)amine, Troxacitabine, Uramustine, Vadimezan, Vinflunine, ZD6126 or Zosuquidar.

The exact method for administering the compound and the additional therapeutic agent will be apparent to one of ordinary skill in the art. In some exemplary embodiments the compound and the additional therapeutic agent are co-administered. In other embodiments, the compound and the additional therapeutic agent are separately administered.

In some embodiments, the compound and the additional therapeutic agent are administered with the second agent simultaneously or separately. This administration in combination can include simultaneous administration of the two agents in the same dosage form, simultaneous administration in separate dosage forms, and separate administration. That is, the compound and any of the additional therapeutic agents described herein can be formulated together in the same dosage form and administered simultaneously. Alternatively, the compound and any of the additional therapeutic agents described herein can be simultaneously administered, wherein both the agents are present in separate formulations. In another alternative, the compound can be administered just followed by any of the additional therapeutic agents described herein, or vice versa. In some embodiments of the separate administration protocol, the compound and any of the additional therapeutic agents described herein are administered a few minutes apart, or a few hours apart, or a few days apart.

### Articles of Manufacture

Also provided herein are articles of manufacture, or “kit”, containing materials useful for the treatment of a cancer provided herein. In one embodiment, the kit comprises a container comprising compound or stereoisomer, atropisomer, tautomer, or pharmaceutically acceptable salt thereof described herein. The kit may further comprise a label or package insert on or associated with the container. Suitable containers include, for example, bottles, vials, syringes, blister pack, etc. The container may be formed from a variety of materials such as glass or plastic. The container may hold a compound or a pharmaceutically acceptable salt thereof described herein or a formulation thereof which is effective for treating the condition and may have a sterile access port (for example, the container may be an intravenous solution bag or a vial having a stopper pierceable by a hypodermic injection needle). At least one active agent in the composition is a compound or a pharmaceutically acceptable salt thereof described herein. Alternatively, or additionally, the article of manufacture may further comprise a second container comprising a pharmaceutical diluent, such as bacteriostatic water for injection (BWFI), phosphate-buffered saline, Ringer's solution or dextrose solution. It may further include other materials desirable from a commercial and user standpoint, including other buffers, diluents, filters, needles, and syringes.

In another embodiment, the kits are suitable for the delivery of solid oral forms of a compound or a pharmaceutically acceptable salt thereof described herein, such as tablets or capsules. Such a kit can include a number of unit dosages. An example of such a kit is a “blister pack”. Blister packs are well known in the packaging industry and are widely used for packaging pharmaceutical unit dosage forms.

### Embodiments

Embodiment No. 1: A compound having formula (I):

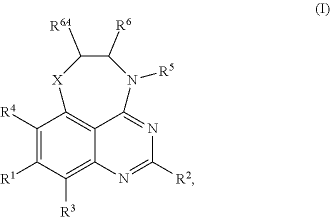

or a stereoisomer, atropisomer, tautomer, or pharmaceutically acceptable salt thereof, wherein;
X is NR¹³, O, C(R^{x})₂, C(O), SO, SO₂, or S;
u is 1 or 2;
each R^{x} is independently hydrogen, halogen, unsubstituted C₁₋₃ alkyl or unsubstituted C₁₋₃ haloalkyl;
or wherein two R^{x} together form a cyclopropyl together with the carbon to which they are bound;
R¹ is R⁷-substituted or unsubstituted indolyl, R⁷-substituted or unsubstituted benzofuranyl, R⁷-substituted or unsubstituted napthyl, R⁷-substituted or unsubstituted indazolyl, R⁷-substituted or unsubstituted indenyl, R⁷-substituted or unsubstituted benzothiazolyl, R^{7A} substituted or unsubstituted phenyl, or R^{7A}-substituted or unsubstituted pyridinyl;
each R⁷ is independently hydrogen, halogen, CN, CH₂OH, —OH, NH₂, N(Me)₂, unsubstituted C₁₋₃ alkyl, unsubstituted C₂₋₅ alkynyl, unsubstituted C₁₋₃ haloalkyl, or unsubstituted cyclopropyl;
each R^{7A} is independently hydrogen, halogen, NH₂, N(Me)₂, unsubstituted C₁₋₃ alkyl, unsubstituted C₁₋₃ haloalkyl, or unsubstituted cyclopropyl; R² is hydrogen, O-L¹-R⁸, R^{8A}-substituted or unsubstituted C₁₋₃ alkyl, or R^{8B}-substituted or unsubstituted 4-10 membered heterocycle;
L¹ is a bond or R^{L1}-substituted or unsubstituted C₁₋₃ alkylene;
R^{L1} is halogen or unsubstituted C₁₋₃ alkyl;
R⁸ is R⁹-substituted or unsubstituted 4-10 membered heterocycle comprising N, S, or O;
each R⁹ is independently halogen, oxo, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle;
   or wherein two R⁹ together form a C₃₋₅ cycloalkyl or 3-5 membered heterocycle; R¹⁰ is hydrogen or halogen;
each R^{8A} is independently R^{9A}-substituted or unsubstituted C₁₋₃ alkyl, R^{9A}-substituted or unsubstituted C₁₋₃ alkoxy, R^{9A}-substituted or unsubstituted C₃₋₄ cycloalkyl, or R^{9A}-substituted or unsubstituted 4-6 membered heterocycle;
each R^{9A} is independently halogen, oxo, unsubstituted C₁₋₃ alkyl, unsubstituted C₁₋₃ haloalkyl, unsubstituted C₁₋₃ alkoxy, unsubstituted C₁₋₃ alkylidene, R⁹-substituted or unsubstituted C₃₋₄ cycloalkyl, or R⁹-substituted or unsubstituted 4-10 membered heterocycle comprising N, S, or O;
R^{8B} is independently halogen, oxo, —NH₂, unsubstituted C₁₋₃ alkyl, unsubstituted C₁₋₃ haloalkyl, unsubstituted C₁₋₃ alkoxy, or unsubstituted C₁₋₃ alkylidene;
R³ and R⁴ are each independently hydrogen, —CN, halogen, unsubstituted C₁₋₃ alkyl, or unsubstituted cyclopropyl;
R⁵ is R^{5A}-substituted or unsubstituted C₁₋₆ alkyl, R^{5A}-substituted or unsubstituted C₁₋₆ haloalkyl, R^{5A}-substituted or unsubstituted C₃₋₁₀ cycloalkyl, R^{5A}-substituted or unsubstituted 3-10 membered heterocycle, or R^{5A}-substituted or unsubstituted 5-10 membered heteroaryl;
each R^{5A} is independently halogen, oxo, CN, OR¹¹, SR¹², SO₂R¹², NR¹³R¹⁴, C(O)N(R¹¹)₂, C(O)R¹¹, R^{5B}-substituted or unsubstituted C₁₋₆ alkyl, R^{5B}-substituted or unsubstituted C₁₋₆ haloalkyl, R^{5B}-substituted or unsubstituted C₃₋₆ cycloalkyl, R^{5B}-substituted or unsubstituted 3-6 membered heterocycle, R^{5B}-substituted or unsubstituted C₅₋₈ aryl, or R^{5B}-substituted or unsubstituted 5-9 membered heteroaryl;
   or wherein two R^{5A} together form a C₃₋₆ cycloalkyl or 3-6 membered heterocycle;
each R^{5B} is independently halogen, oxo, CN, OR¹, NR¹³R¹⁴, SR¹², SO₂R¹², C(O)N(R¹¹)₂, C(O)R¹¹, R^{5C}-substituted or unsubstituted C₁₋₃ alkyl, R^{5C}-substituted or unsubstituted C₁₋₃ haloalkyl, R^{5C}-substituted or unsubstituted C₃₋₆ cycloalkyl, R^{5C}-substituted or unsubstituted 3-6 membered heterocycle, R^{5C}-substituted or unsubstituted phenyl, or R^{5C}-substituted or unsubstituted 5-6 membered heteroaryl;
   or wherein two R^{5B} together form a C₃₋₄ cycloalkyl or 3-6 membered heterocycle;
each R^{5C} is independently halogen, oxo, CN, C(O)CH₃, C(O)NH₂, OH, OCH₃, CF₃, CHF₂, CH₂F, NR¹³R¹⁴, SCH₃, SO₂NH₂, SO₂CH₃, unsubstituted C₁₋₃ alkyl, unsubstituted C₁₋₃ haloalkyl, unsubstituted C₃₋₄ cycloalkyl, or unsubstituted 3-4 membered heterocycle;
each R¹¹ is independently hydrogen, unsubstituted C₁₋₃ alkyl, unsubstituted C₁₋₃ haloalkyl, unsubstituted C₃₋₄ cycloalkyl, or unsubstituted 3-4 membered heterocycle;
each R¹² is independently NH₂ or unsubstituted C₁₋₃ alkyl;
each R¹³ and R¹⁴ are independently hydrogen, C(O)R¹¹, C(O)N(R¹¹)₂, R¹⁵-substituted or unsubstituted C₁₋₆ alkyl, R¹⁵-substituted or unsubstituted C₃₋₆ cycloalkyl, or R¹⁵-substituted or unsubstituted 3-6 membered heterocycle;
each R¹⁵ is halogen, CN, C(O)CH₃, C(O)NH₂, OH, OCH₃, CF₃, CHF₂, CH₂F, NH₂, NHCH₃, N(CH₃)₂, SO₂NH₂, SO₂CH₃, R¹⁰-substituted or unsubstituted C₁₋₃ alkyl, R¹⁶-substituted or unsubstituted C₃₋₄ cycloalkyl, R¹⁶-substituted or unsubstituted 3-6 membered heterocycle, R¹⁶-substituted or unsubstituted 5-9 membered aryl, or R¹⁶-substituted or unsubstituted 5-9 membered heteroaryl;
each R¹⁶ is independently halogen, CN, C(O)CH₃, C(O)NH₂, OH, OCH₃, CF₃, CHF₂, CH₂F, NH₂, NHCH₃, N(CH₃)₂, SO₂NH₂, SO₂CH₃, R¹⁷-substituted or unsubstituted C₁₋₃ alkyl, R¹⁷-substituted or unsubstituted C₃₋₆ cycloalkyl, R¹⁷-substituted or unsubstituted 3-6 membered heterocycle, R¹⁷-substituted or unsubstituted 5-9 membered aryl, or R¹⁷-substituted or unsubstituted 5-9 membered heteroaryl;
each R¹⁷ is independently halogen, CN, C(O)CH₃, C(O)NH₂, OH, OCH₃, CF₃, CHF₂, CH₂F, NH₂, NHCH₃, N(CH₃)₂, SO₂NH₂, SO₂CH₃, or unsubstituted C₁₋₃ alkyl;
R⁶ and R^{6A} are independently hydrogen, halogen, NR¹³R¹⁴, or R^{6B}-substituted or unsubstituted C₁₋₆ alkyl; and
R⁶⁸ is halogen, CN, OH, OCH₃, CF₃, CHF₂, CH₂F, or unsubstituted C₁₋₃ alkyl.
Embodiment No. 2: The compound of embodiment 1, wherein R¹ is R^{7A}-substituted or unsubstituted phenyl, R⁷-substituted or unsubstituted indazolyl, or R^{7A}-substituted or unsubstituted pyridinyl.
Embodiment No. 3: The compound of embodiment 1, wherein R¹ is R^{7A}-substituted or unsubstituted phenyl.
Embodiment No. 4: The compound of embodiment 1, wherein R¹ is R⁷-substituted or unsubstituted indazolyl.
Embodiment No. 5: The compound of embodiment 1, wherein R¹ is R^{7A}-substituted or unsubstituted pyridinyl.
Embodiment No. 6: The compound of any one of embodiments 1-5, wherein each R^{7A} is independently halogen, NH₂, unsubstituted C₁₋₃ alkyl, or unsubstituted C₁₋₃ haloalkyl.
Embodiment No. 7: The compound of embodiment 1 or embodiment 2, wherein R¹ is

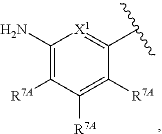

wherein,
X¹ is N, CH, or CF; and
R^{7A} is hydrogen, halogen, unsubstituted C₁₋₃ alkyl, or unsubstituted C₁₋₃ haloalkyl.
Embodiment No. 8: The compound of any one of embodiments 1, 2, 5, or 7, wherein R¹ is

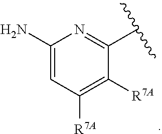

Embodiment No. 9: The compound of any one of embodiments 1, 2, 5, 7, or 8, wherein R¹ is

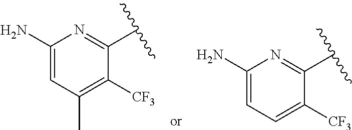 [SMILES: *c1nc(N)cc(C)c1C(F)(F)F ; *c1nc(N)ccc1C(F)(F)F]

Embodiment No. 10: The compound of any one of embodiments 1-3 or 7, wherein R¹ is

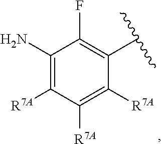

wherein R^{7A} is hydrogen, halogen, unsubstituted C₁₋₃ alkyl or unsubstituted C₁₋₃ haloalkyl.
Embodiment No. 11: The compound of any one of embodiments 1-4, 8, or 11, wherein R¹ is

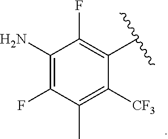 [SMILES: *c1c(F)c(N)c(F)c(C)c1C(F)(F)F]

Embodiment No. 12: The compound of embodiment 1, wherein R¹ is

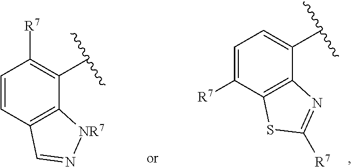

wherein each R⁷ is independently halogen, NH₂, N(Me)₂, unsubstituted C₁₋₃ alkyl, or unsubstituted C₁₋₃ haloalkyl.
Embodiment No. 13: The compound of any one of embodiments 1-12, wherein R² is O-L¹-R⁸, R^{8B}-substituted or unsubstituted C₁₋₃ alkyl, or R^{8B}-substituted or unsubstituted 4-6 membered heterocycle.
Embodiment No. 14: The compound of any one of embodiments 1-13, wherein R² is O-L¹-R⁸.
Embodiment No. 15: The compound of any one of embodiments 13-14, wherein L¹ is unsubstituted C₁₋₃ alkylene.
Embodiment No. 16: The compound of any one of embodiments 13-15, wherein R⁸ is 4-10 membered heterocycle comprising one N heteroatom.
Embodiment No. 17: The compound of any one of embodiments 13-16, wherein R⁸ is

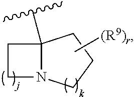

wherein,
R⁹ is halogen or R¹⁰-substituted or unsubstituted C₁₋₃ alkylidene
r is an integer of 0-12;
j is 1, 2, or 3; and
k is 1 or 2.
Embodiment No. 18: The compound of embodiment 17, wherein r is 0, 1, 2, or 3.
Embodiment No. 19: The compound of anyone of embodiments 13-18, wherein R⁸ is

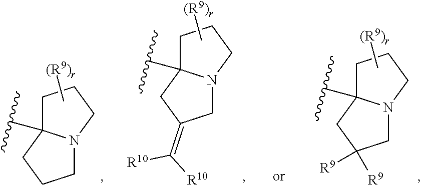

wherein,
R⁹ is independently halogen or R¹⁰-substituted or unsubstituted C₁₋₃ alkylidene;
each R¹⁰ is independently hydrogen or halogen; and
r is 1 or 2.
Embodiment No. 20: The compound of any one of embodiments 13-16, wherein R⁸ is

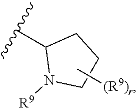

wherein,
R⁹ is independently halogen, oxo, or unsubstituted C₁₋₃ alkyl;
or wherein two R⁹ together form a C₃₋₅ cycloalkyl or 3-5 membered heterocycle; and
r is 1 or 2.
Embodiment No. 21: The compound of any one of embodiments 13-16, wherein R⁸ is

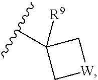

wherein
R⁹ is hydrogen or unsubstituted C₁₋₃ alkyl;
W is O, SO₂, or NR¹²; and
R¹² is hydrogen, unsubstituted C₁₋₃ alkyl, or unsubstituted C₁₋₃ haloalkyl.
Embodiment No. 22: The compound of any one of embodiments 13-16 or 21, wherein R^{a} is azetidinyl, oxetanyl, or thietanedioxide.
Embodiment No. 23: The compound of any one of embodiments 1-22, wherein R² is

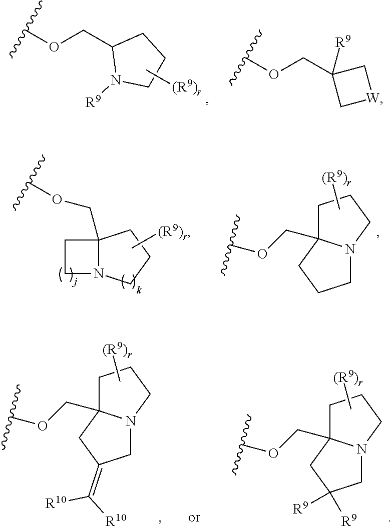

Embodiment No. 24: The compound of embodiment 23, wherein R⁹ is halogen or R¹⁰-substituted or unsubstituted C₁₋₃ alkylidene.
Embodiment No. 25: The compound of any one of embodiments 1-12, wherein R² is hydrogen.
Embodiment No. 26: The compound of any one of embodiments 1-25, wherein R³ is hydrogen or halogen.
Embodiment No. 27: The compound of any one of embodiments 1-26, wherein R⁴ is halogen.
Embodiment No. 28: The compound of any one of embodiments 1-27, wherein R⁵ is R^{5A}-substituted or unsubstituted C₁₋₄ alkyl.
Embodiment No. 29: The compound of any one of embodiments 1-28, wherein R⁵ is

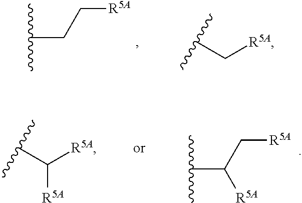

Embodiment No. 30: The compound of any one of embodiments 1-29, wherein R⁵ is

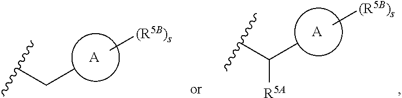

wherein
Ring A is a 3-6 membered heterocycle or 5-9 membered heteroaryl comprising at least one N heteroatom; and
s is 0, 1, 2, or 3.
Embodiment No. 31: The compound of embodiment 30, wherein Ring A is azetidinyl, thietanyl 1,1-dioxide, imidazolyl, thiazolyl, isothiazolyl, triazolyl, pyrazolyl, pyrazinyl, pyridonyl, pyridinyl, pyrimidinyl, pyridazinyl, pyrrolopyridinyl, or pyrazolopyridinyl.
Embodiment No. 32: The compound of embodiment 30 or 31, wherein Ring A is imidazolyl, isothiazolyl, or triazolyl.
Embodiment No. 33: The compound of embodiment 30 or 31, wherein Ring A is pyrazolyl, pyridonyl, pyridinyl, pyrimidinyl, or pyridazinyl.
Embodiment No. 34: The compound of embodiment 30 having the formula:

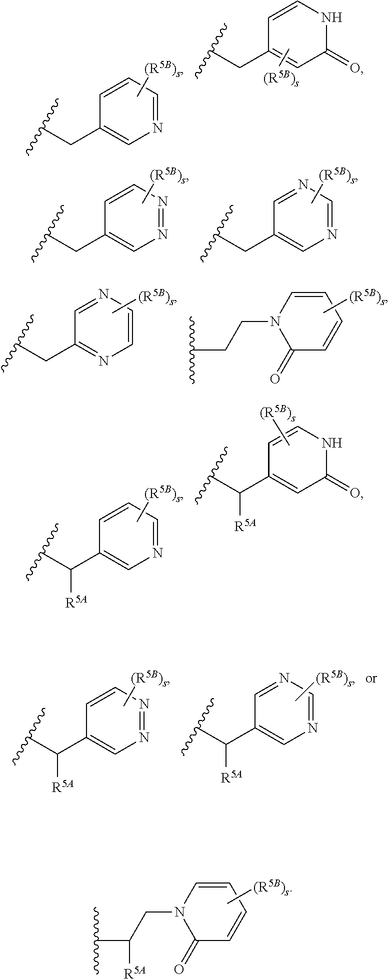

Embodiment No. 35: The compound of any one of embodiments 1-34, wherein two R^{5A} together form a C₃₋₄ cycloalkyl or 3-4 membered heterocycle.
Embodiment No. 36: The compound of any one of embodiments 1-29, wherein R⁵ is

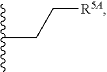

wherein
R^{5A} is CN, OH, COR¹¹, SO₂R¹², NR¹³R¹⁴, R^{5B}-substituted or unsubstituted azetidinyl, or R^{M}-substituted or unsubstituted oxetanyl.
Embodiment No. 37: The compound of any one of embodiments 1-27, wherein R⁵ is R^{5A}-substituted or unsubstituted 5-9 membered heteroaryl.
Embodiment No. 38: The compound of embodiment 1 having the formula:

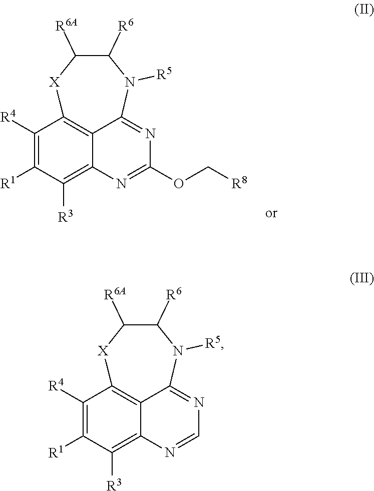

or a stereoisomer, atropisomer, tautomer, or pharmaceutically acceptable salt thereof.
Embodiment No. 39: The compound of embodiment 1 having the formula:

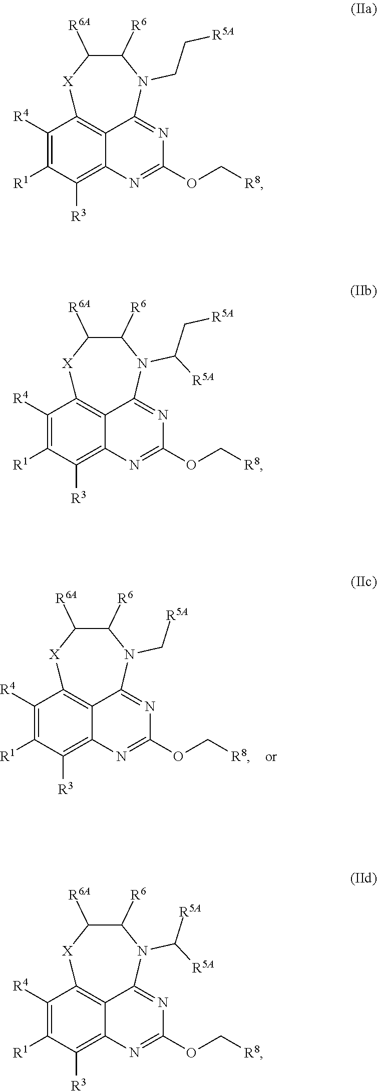

or a stereoisomer, atropisomer, tautomer, or pharmaceutically acceptable salt thereof.
Embodiment No. 40: The compound of embodiment 1 having the formula:

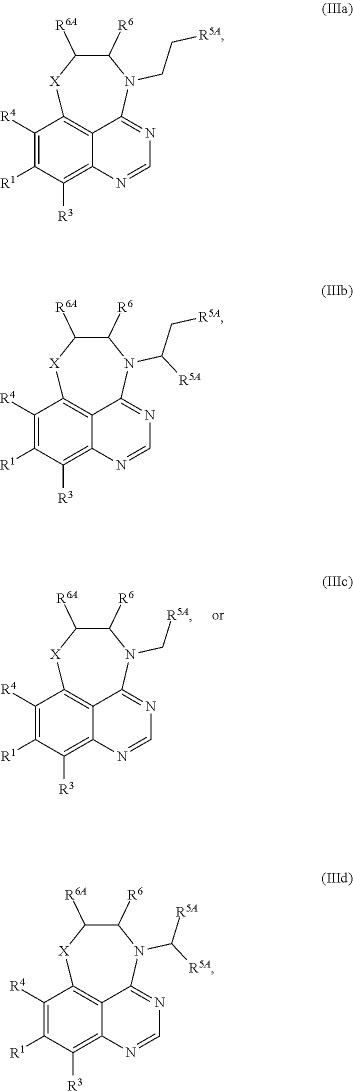

or a stereoisomer, atropisomer, tautomer, or pharmaceutically acceptable salt thereof.
Embodiment No. 41: The compound of embodiment 1 having the formula:

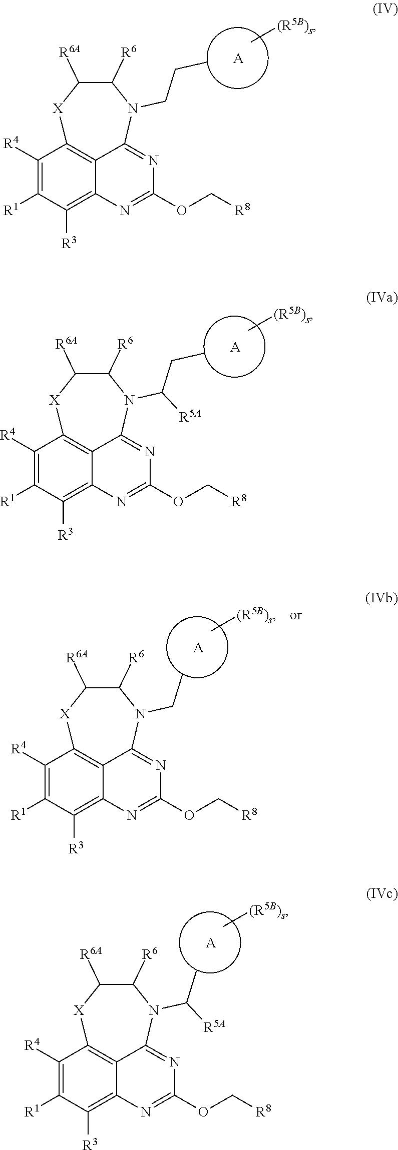

or a stereoisomer, atropisomer, tautomer, or pharmaceutically acceptable salt thereof.
Embodiment No. 42: The compound of embodiment 1 having the formula:

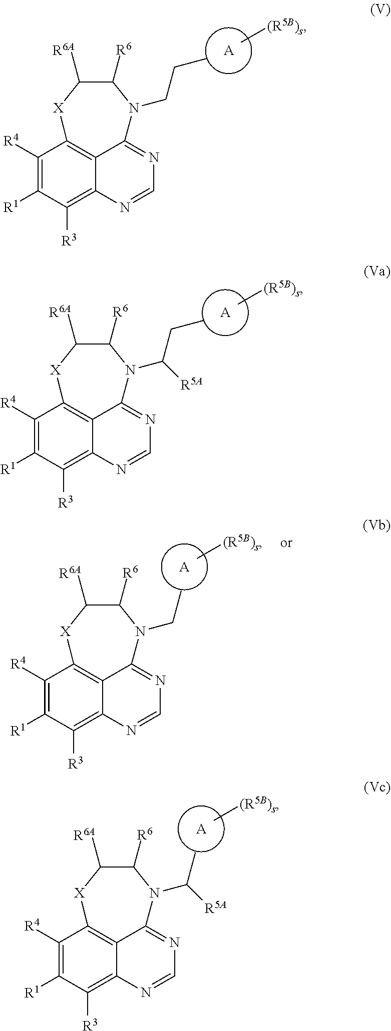

or a stereoisomer, atropisomer, tautomer, or pharmaceutically acceptable salt thereof.
Embodiment No. 43: The compound of any one of embodiments 1-42, wherein R⁸ is:

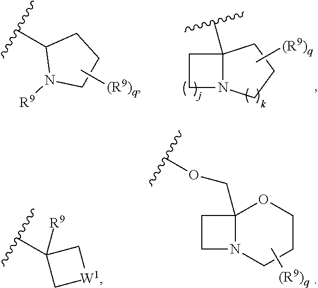

Embodiment No. 44: The compound of any one of embodiments 1-42, wherein R⁸ is:

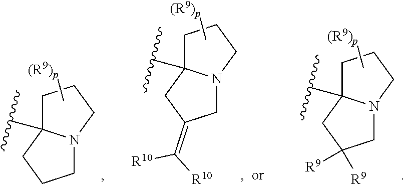

Embodiment No. 45: The compound of any one of embodiments 1-42, wherein R⁸ is:

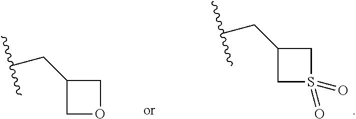 [SMILES: *CC1COC1 ; *CC1CS(=O)(=O)C1]

Embodiment No. 46: The compound of any one of embodiments 1-45, wherein X is O.
Embodiment No. 47: The compound of any one of embodiments 1-45, wherein X is C(R^{x})₂.
Embodiment No. 48: The compound any one of embodiments 1-47, wherein Re is R^{6A}-substituted or unsubstituted C₁₋₃ alkyl.
Embodiment No. 49: The compound any one of embodiments 1-47, wherein R⁶ is R^{6A}-substituted C₁₋₃ alkyl.
Embodiment No. 50: The compound of embodiment 48 or 49, wherein R^{6A} is halogen, CN, or OH.
Embodiment No. 51: The compound any one of embodiments 1-47, wherein R⁶ is hydrogen.
Embodiment No. 52: A compound of Table 1 or a stereoisomer, atropisomer, tautomer, or pharmaceutically acceptable salt thereof.
Embodiment No. 53: A compound of Table 2 or a stereoisomer, atropisomer, tautomer, or pharmaceutically acceptable salt thereof.
Embodiment No. 54: A pharmaceutical composition comprising a compound or a stereoisomer, atropisomer, tautomer, or pharmaceutically acceptable salt thereof of any one of embodiments 1-53 and one or more pharmaceutically acceptable excipients.
Embodiment No. 55: A method of treating cancer, the method comprising administering an effective amount of a compound or a stereoisomer, atropisomer, tautomer, or pharmaceutically acceptable salt thereof of any one of embodiments 1-53 or a pharmaceutical composition of embodiment 54.
Embodiment No. 56: The method of embodiment 55, wherein the cancer is characterized as comprising a KRas mutation.
Embodiment No. 57: The method of embodiment 56, wherein the KRas mutation corresponds to a KRas^{G12D} mutation or KRas^{G12V} mutation.
Embodiment No. 58: The method of embodiment 56, further comprising testing a sample from the patient before administration for the absence or presence of a KRas mutation.
Embodiment No. 59: The method of embodiment 58, wherein the compound, stereoisomer, atropisomer, tautomer, or pharmaceutically acceptable salt thereof or pharmaceutical composition is administered to the patient after the patient sample shows the presence of a KRas mutation.
Embodiment No. 60: The method of any one of embodiments 55-59, wherein the cancer is tissue agnostic.
Embodiment No. 61: The method of any one of embodiments 55-59, wherein the cancer is pancreatic cancer, lung cancer, or colorectal cancer.
Embodiment No. 62: The method of embodiment 61, wherein the lung cancer is lung adenocarcinoma, NSCLC, or SCLC.
Embodiment No. 63: The method of embodiment 61, wherein the cancer is pancreatic cancer.
Embodiment No. 64: The method of embodiment 61, wherein the cancer is colorectal cancer.
Embodiment No. 65: The method of any one of embodiments 55-64, further comprising administering at least one additional therapeutic agent.
Embodiment No. 66: The method of embodiment 65, wherein the additional therapeutic agent comprises an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor, a taxane, an anti-metabolite agent, or an alkylating agent.
Embodiment No. 67: A compound according to any one of embodiments 1-53, or a stereoisomer, atropisomer, tautomer, or pharmaceutically acceptable salt thereof, for use as therapeutically active substance.
Embodiment No. 68: The use of a compound according to any one of embodiments 1-53, or stereoisomer, atropisomer, tautomer, or pharmaceutically acceptable salt thereof, for the therapeutic treatment of a cancer comprising a KRas mutation.
Embodiment No. 69: The use of a compound according to any one of embodiments 1-53, or stereoisomer, atropisomer, tautomer, or pharmaceutically acceptable salt thereof, for the preparation of a medicament for the therapeutic treatment of a cancer comprising a KRas mutation.
Embodiment No. 70: Use of a compound of any one of embodiments 1-53, or stereoisomer, atropisomer, tautomer, or pharmaceutically salt thereof, in the manufacture of a medicament for inhibiting tumor metastasis.
Embodiment No. 71: A compound according to any one of embodiments 1-53, or stereoisomer, atropisomer, tautomer, or pharmaceutically salt thereof, for the therapeutic and/or prophylactic treatment of a cancer comprising a KRas mutation.
Embodiment No. 72: A method for regulating activity of a KRas mutant protein, the method comprising reacting the mutant protein with a compound of any one of embodiments 1-53, or stereoisomer, atropisomer, tautomer, or pharmaceutically acceptable salt thereof.
Embodiment No. 73: A method for inhibiting proliferation of a cell population, the method comprising contacting the cell population with the compound of any one of embodiments 1-53, or stereoisomer, atropisomer, tautomer, or pharmaceutically acceptable salt thereof.
Embodiment No. 74: The method of embodiment 73, wherein the inhibition of proliferation is measured as a decrease in cell viability of the cell population.
Embodiment No. 75: A method for preparing a labeled KRas mutant protein, the method comprising reacting a KRas mutant protein with a labeled compound of any one of embodiments 1-56, or stereoisomer, atropisomer, tautomer, or pharmaceutically acceptable salt thereof, to result in the labeled KRas mutant protein.
Embodiment No. 76: A method for inhibiting tumor metastasis comprising administering to an individual in need thereof a therapeutically effective amount of the compound of any one of embodiments 1-53, or stereoisomer, atropisomer, tautomer, or pharmaceutically acceptable salt thereof or a pharmaceutical composition of embodiment 54 to a subject in need thereof.
Embodiment No. 77: A process for synthesizing a compound of formula or (1) as set forth herein.

### EXAMPLES

The following examples illustrate the preparation and biological evaluation of compounds within the scope of the invention. These examples and preparations which follow are provided to enable those skilled in the art to more clearly understand and to practice the present invention. They should not be considered as limiting the scope of the invention, but merely as being illustrative and representative thereof.

### Intermediate 1A: ((2R,7aS)-2-fluorotetrahydro-1H-pyrrolizin-7a(5H)-yl)methanol

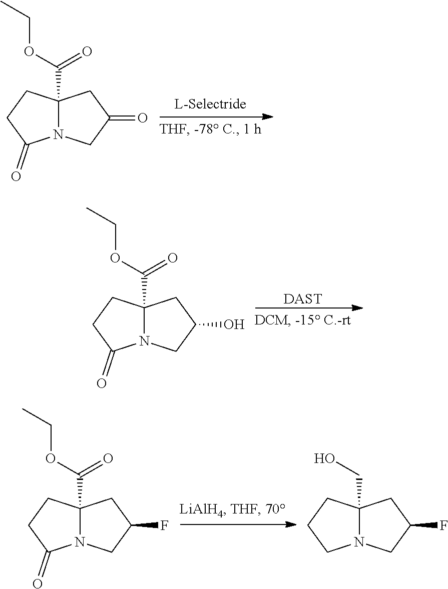

Step 1: ethyl (2S,7aS)-2-hydroxy-5-oxotetrahydro-1H-pyrrolizine-7a(5H)-carboxylate

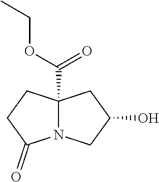

A solution of ethyl (8S)-3,6-dioxo-1,2,5,7-tetrahydropyrrolizine-8-carboxylate (10.00 g, 47.3 mmol) in Tetrahydrofuran (10 mL) was added L-selectride (1M in THF) (23.6 mL, 23.6 mmol) and the resulting mixture was stirred at −78° C. for 20 minutes. Then additional Lithium L-selectride (1M in THF) (23.6 mL, 23.6 mmol) was added and the resulting mixture was stirred at −78° C. for 40 minutes. The reaction was quenched with saturated sodium bicarbonate solution. The solution was concentrated under vacuum to remove THF. Then and the residue was diluted with dichloromethane/methanol (20/1). After filtration, the solids were removed and the filtrate was collected and concentrated under reduced pressure. The crude product was purified by flash chromatography on silica gel eluting with dichloromethane/methanol (97/3) to afford ethyl (2S,7aS)-2-hydroxy-5-oxotetrahydro-1H-pyrrolizine-7a(5H)-carboxylate (4 g, 18.759 mmol, 39.6% yield) as a yellow oil.

LC-MS: (ESI, m/z): [M+H]⁺=214.1.

Step 2: ethyl (2R,7aS)-2-fluoro-5-oxotetrahydro-1H-pyrrolizine-7a(5H)-carboxylate

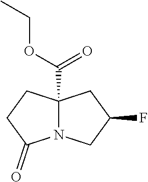

Under nitrogen, to a solution of ethyl ethyl (2S,7aS)-2-hydroxy-5-oxotetrahydro-1H-pyrrolizine-7a(5H)-carboxylate (4 g, 18.6 mmol) in dichloromethane (40 mL) was added diethylaminosulfurtrifluoride (4.2 mL, 37.2 mmol) at −15° C. The solution was stirred at room temperature for 16 hours. After completion, the reaction was quenched with ethanol and the solvent was concentrated under vacuum. The residue was diluted with ethyl acetate, washed with water, dried over anhydrous sodium sulfate and concentrated under vacuum. The residue was purified by flash chromatography on silica gel eluting with petroleum ether/ethyl acetate (7/3) to afford ethyl (2R,7aS)-2-fluoro-5-oxotetrahydro-1H-pyrrolizine-7a(5H)-carboxylate (2.2 g, 10.1 mmol, 54.3% yield) as a yellow oil. LC-MS: (ESI, m/z): [M+H]⁺=216.1.

Step 3: ((2R,7aS)-2-fluorotetrahydro-1H-pyrrolizin-7a(5H)-yl)methanol

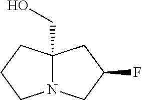 [SMILES: OC[C@@]12CCCN1C[C@H](F)C2]

A solution of ethyl (2R,7aS)-2-fluoro-5-oxotetrahydro-1H-pyrrolizine-7a(5H)-carboxylate (10.0 g, 46.2 mmol) in tetrahydrofuran (100 mL) was added lithium aluminum hydride (1M in THF) (138.6 mL, 138.6 mmol) at 0° C. Then the mixture was stirred for 30 minutes at 70° C. (extended reaction time will lead to the F-eliminated byproduct). After completion, the reaction was quenched with sodium sulfate decahydrate and diluted with tetrahydrofuran. After filtration, the filtrate was collected and the solid was washed with tetrahydrofuran for three times. The tetrahydrofuran in the filtrate was blew out by nitrogen gas (concentration under vacuum will cause loss of product with low boiling point) to afforded ((2R,7aS)-2-fluorotetrahydro-1H-pyrrolizin-7a(5H)-yl)methanol (5.3 g, 33.1 mmol, 71.6% yield) as a light yellow oil. LC-MS: (ESI, m/z): [M+H]⁺=160.1.

### Intermediate 2A: 7-bromo-6-chloro-5-fluoroquinazolin-4(3H)-one

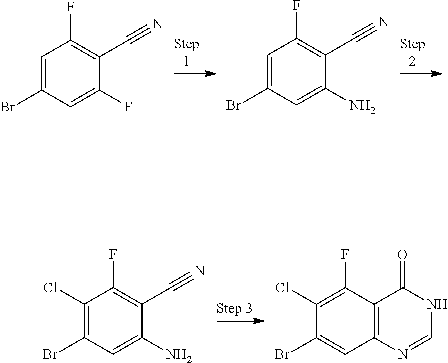 [SMILES: N#Cc1c(F)cc(Br)cc1F ; N#Cc1c(N)cc(Br)cc1F ; N#Cc1c(N)cc(Br)c(Cl)c1F ; O=c1[nH]cnc2cc(Br)c(Cl)c(F)c12]

Step 1: 2-amino-4-bromo-6-fluorobenzonitrile

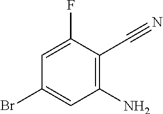 [SMILES: N#Cc1c(N)cc(Br)cc1F]

To a solution of 4-bromo-2,6-difluorobenzonitrile (4000.0 g, 435.7 mmol) in i-PrOH (40.0 L) was added NH₃·H₂O (20.0 L) and was stirred for 6 h at 80° C. in high pressure tank. The resulting solution was evaporated until 20 L remained. The solids were collected by filtration and dried to afford 3625 g (91%) title compound as a white solid. LCMS (ESI): [M−H]+=213.

Step 2: 6-amino-4-bromo-3-chloro-2-fluorobenzonitrile

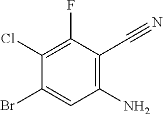 [SMILES: N#Cc1c(N)cc(Br)c(Cl)c1F]

To a solution of 2-amino-4-bromo-6-fluorobenzonitrile (450.0 g, 2102.8 mmol) in DMF (2.5 L) was added NCS (280.7 g, 2102.8 mmol) at 0° C. and was stirred for 2 h at 60° C. The resulting solution was cooled to room temperature and poured into 25 L of water. The solids were collected by filtration. The solid was added to 3.0 L of ethyl acetate/petroleum ether (1:5) and was stirred for 30 min at 25° C. The solids were collected by filtration to afford 350 g crude title compound. The 350 g crude compound was added to 1.5 L of ethyl acetate/petroleum ether (1:10) and was stirred for 30 min at 25° C. The solids were collected by filtration to afford 210 g (40% yield) title compound as a yellow solid. LCMS (ESI): [M−H]⁺=247.

Step 3: 7-bromo-6-chloro-5-fluoroquinazolin-4(3H)-one

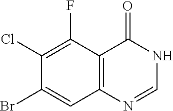 [SMILES: O=c1[nH]cnc2cc(Br)c(Cl)c(F)c12]

To a solution of 6-amino-4-bromo-3-chloro-2-fluorobenzonitrile (15.0 g, 150.5 mmol) in formic acid (75.0 mL) was added H₂SO₄ (7.5 mL) at 25° C. and was stirred for 30 min at 100° C. The resulting solution was cooled to room temperature and poured into 250 mL of ice/water. The solids were collected by filtration and dried to afford 12.24 g (73%) title compound as an off-white solid. LCMS (ESI): [M−H]⁺=277. ¹H NMR (300 MHz, DMSO-d₆) δ 12.55 (s, 1H), 8.14 (s, 1H), 7.92 (d, J=2.1 Hz, 1H).

### Intermediate 3A: 7-bromo-2,6-dichloro-5-fluoroquinazolin-4(3H)-one

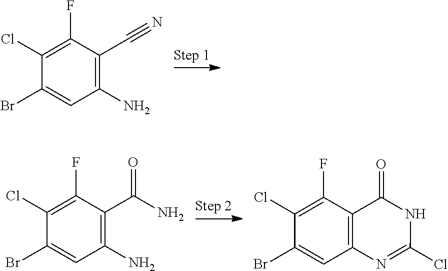 [SMILES: N#Cc1c(N)cc(Br)c(Cl)c1F ; NC(=O)c1c(N)cc(Br)c(Cl)c1F ; O=c1[nH]c(Cl)nc2cc(Br)c(Cl)c(F)c12]

Step 1: 6-amino-4-bromo-3-chloro-2-fluorobenzamide

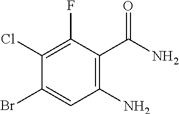 [SMILES: NC(=O)c1c(N)cc(Br)c(Cl)c1F]

To a solution of 6-amino-4-bromo-3-chloro-2-fluorobenzonitrile (600 g, 2405 mmol) in DMSO (3.0 L) was added K₂CO₃ (665 g, 4810 mmol). Then H₂O₂ (30%) (1091 g, 9620 mmol) was added dropwise at 15° C. and was stirred for 30 min at 25° C. The reaction was then quenched by the addition of 3 L of saturated sodium sulfite aqueous. The solids were collected by filtration and washed by water. The solid was dried to afford 512 g (79%) title compound as a yellow solid. LCMS (ESI): [M+H]⁺=267.

Step 2: 7-bromo-2,6-dichloro-5-fluoroquinazolin-4(3H)-one

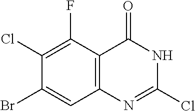 [SMILES: O=c1[nH]c(Cl)nc2cc(Br)c(Cl)c(F)c12]

To a solution of 6-amino-4-bromo-3-chloro-2-fluorobenzamide (16.5 g, 61.7 mmol) in dioxane (100.0 mL) was added thiophosgene (14.9 g, 129.6 mmol) dropwise at 0° C. and then stirred for 1 h at room temperature. Then the mixture was stirred for 50 min at 105° C. The reaction mixture was cooled to room temperature and was concentrated in vacuo. To the solid was added dioxane (40 mL) and MTBE (50 mL) and then stirred for 15 min. The solids were collected by filtration to afford 9.22 g (47%) title compound as an off white solid. LCMS (ESI): [M−H]⁺=309. ¹H NMR (300 MHz, DMSO-d₆) δ 7.90 (d, J=1.8 Hz, 1H).

### Intermediate 4A. 7-(6-(bis(4-methoxybenzyl)amino)-4-methyl-3-(trifluoromethyl)pyridin-2-yl)-6-chloro-5-fluoroquinazolin-4(3H)-one

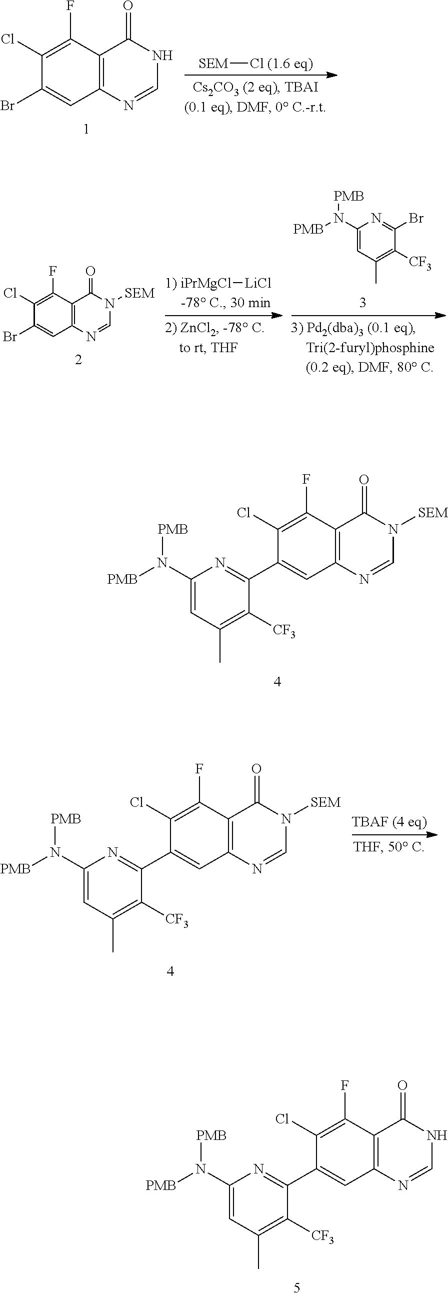

Step 1:7-bromo-6-chloro-5-fluoro-3-((2-(trimethylsilyl)ethoxy)methyl)quinazolin-4(3H)-one

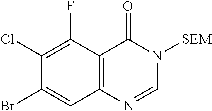 [SMILES: C[Si](C)(C)CCOCn1cnc2cc(Br)c(Cl)c(F)c2c1=O]

A solution of 7-bromo-6-chloro-5-fluoro-3H-quinazolin-4-one (20.00 g, 72.1 mmol), tetrabutylazanium iodide (2.66 g, 7.2 mmol) and cesium carbonate (46.97 g, 144.2 mmol) in N,N-dimethylformamide (160 mL) was stirred at 0° C. for 5 minutes. Then 2-2-(trimethylsilyl)ethoxymethyl chloride (20.4 mL, 115.3 mmol) was added and stirred at 25° C. for 1.5 hours. After completion, the reaction mixture was diluted with water (300 mL). The resulting solution was extracted with ethyl acetate (3×200 mL) and the organic layers were combined. The organic layers were washed with water (3×150 mL) again. The organic layer was dried over anhydrous sodium sulfate and concentrated under vacuum. The residue was purified by flash chromatography on silica gel eluting with ethyl acetate/petroleum ether (1/9) to afford 7-bromo-8-chloro-5-fluoro-3-(2-trimethylsilylethoxymethyl)quinazolin-4-one (24.00 g, 58.86 mmol, 81.7% yield) as a white solid. LC-MS: (ESI, m/z): 407.0 [M+H]⁺

Step 2: 7-(6-(bis(4-methoxybenzyl)amino)-4-methyl-3-(trifluoromethyl)pyridin-2-yl)-6-chloro-5-fluoro-3-((2-(trimethylsilyl)ethoxy)methyl)quinazolin-4(3H)-one

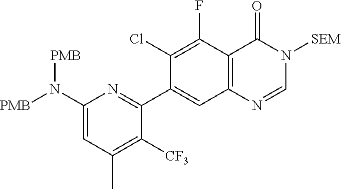

Under nitrogen, a solution of 7-bromo-6-chloro-5-fluoro-3-(2-trimethylsilylethoxymethyl)quinazolin-4-one (10.00 g, 24.5 mmol) in tetrahydrofuran (80 mL) was added isopropylmagnesium chloride lithium chloride complex (1.3 M in tetrahydrofuran) (22.6 mL, 29.4 mmol) at −78° C. and stirred at −78° C. for 0.5 hours. Then zinc chloride (2 M in tetrahydrofuran) (14.7 mL, 29.4 mmol) was added and stirred at 25° C. for 1 hour. The mixture was transferred into a degassed solution of 6-bromo-N,N-bis[(4-methoxyphenyl)methyl]-4-methyl-5-(trifluoromethyl)pyridin-2-amine (10.93 g, 22.1 mmol), tis(dibenzylideneacetone)dipalladium (2.25 g, 2.4 mmol) and tri(2-furyl)phosphine (1.14 g, 4.9 mmol) in N,N-dimethylformamide (20 mL). Then the solution was stirred at 80° C. for 1 hour. After completion, the reaction mixture was concentrated under reduced pressure and then diluted with water (100 mL). The resulting solution was extracted with ethyl acetate (3×200 mL) and the organic layers were combined. The organic layers were washed with water (3×50 mL) again. The organic layer was dried over anhydrous sodium sulfate and concentrated under vacuum. The residue was purified by flash chromatography on silica gel eluting with ethyl acetate/petroleum ether (1/8) to afford 7-[6-[bis[(4-methoxyphenyl)methyl]amino]-4-methyl-3-(trifluoromethyl)-2-pyridyl]-6-chloro-5-fluoro-3-(2-trimethylsilylethoxymethyl)quinazolin-4-one (7.00 g, 9.4 mmol) as a white solid. LC-MS: (ESI, m/z): 743.3 [M+H]⁺

Step 3: 7-(6-(bis(4-methoxybenzyl)amino)-4-methyl-3-(trifluoromethyl)pyridin-2-yl)-6-chloro-5-fluoroquinazolin-4(3H)-one

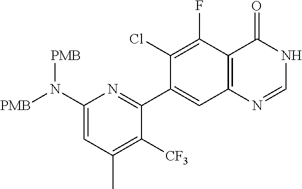

A solution of 7-[6-[bis[(4-methoxyphenyl)methyl]amino]-4-methyl-3-(trifluoromethyl)-2-pyridyl]-6-chloro-5-fluoro-3-(2-trimethylsilylethoxymethyl)quinazolin-4-one (14.00 g, 18.8 mmol) and tetrabutylammonium fluoride (19.70 g, 75.3 mmol) in tetrahydrofuran (90 mL) was stirred at 50° C. for 5 hours. After completion, the reaction mixture was concentrated under reduced pressure. The reaction mixture was diluted with ethyl acetate (300 mL). The resulting solution was washed with water (10×60 mL). The organic layer was dried over anhydrous sodium sulfate and concentrated under vacuum. The residue was purified by flash chromatography on silica gel eluting with ethyl acetate/dichloromethane (1/5) to afford 7-[6-[bis[(4-methoxyphenyl)methyl]amino]-4-methyl-3-(trifluoromethyl)-2-pyridyl]-6-chloro-5-fluoro-3H-quinazolin-4-one (7.50 g, 9.6 mmol) as a white solid. LC-MS: (ESI, m/z): 613.2 [M+H]⁺

### Intermediate 5A. 6-(8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

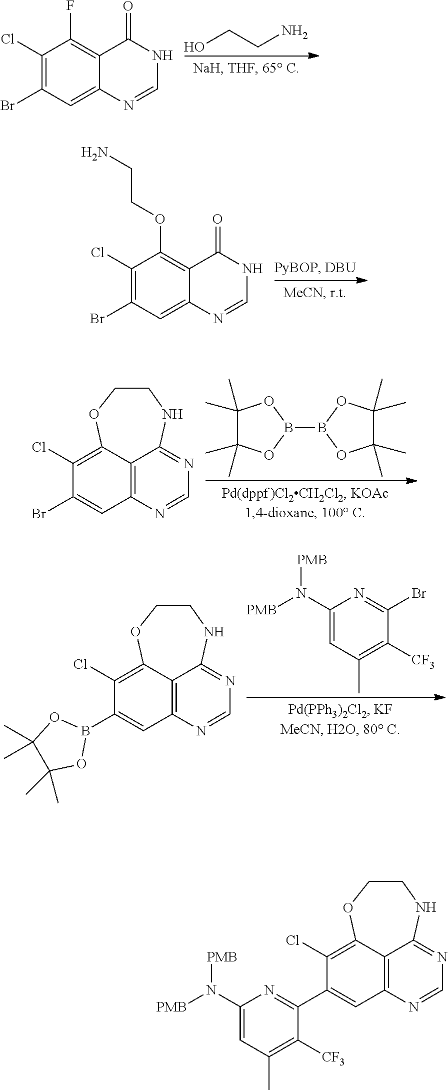

Step 1: 5-(2-aminoethoxy)-7-bromo-6-chloroquinazolin-4(3H)-one

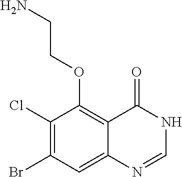 [SMILES: NCCOc1c(Cl)c(Br)cc2nc[nH]c(=O)c12]

A solution of 2-aminoethan-1-ol (2.20 g, 36.04 mmol) and NaH (60% purity) (2.88 g, 72.08 mmol) in Tetrahydrofuran (30 mL) was stirred at 0° C. for 5 minutes. Then 7-bromo-6-chloro-5-fluoro-3H-quinazolin-4-one (5.00 g, 18.02 mmol) was added and stirred at 65° C. for 1 hour. After completion, the reaction mixture was adjusted to PH=7-8 with 1N hydrochloric acid. The solvent was concentrated under vacuum. The residue was purified by reverse-phase chromatography eluting with acetonitrile/water (1:4) to afford 5-(2-aminoethoxy)-7-bromo-6-chloro-3H-quinazolin-4-one (5.70 g, 17.89 mmol, 99.3% yield) as a white solid. LC-MS: (ESI, m/z): 318.5 [M+H]⁺.

Step 2: 9-bromo-8-chloro-5,6-dihydro-4H-[1,4]oxazepino[5,6,7-de]quinazoline

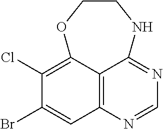 [SMILES: Clc1c(Br)cc2ncnc3c2c1OCCN3]

A solution of 5-(2-aminoethoxy)-7-bromo-6-chloro-3H-quinazolin-4-one (5.80 g, 18.21 mmol) in acetonitrile (70 mL) was added Benzotriazole-1-yl-oxytripyrrolidinophosphonium hexafluorophosphate (11.37 g, 21.85 mmol) and 1,8-Diazabicyclo[5.4.0]undec-7-ene (8.32 g, 54.62 mmol) and stirred at 25° C. for 2 hours. After completion, the reaction mixture was diluted with ethyl acetate, washed with water, dried over anhydrous sodium sulfate and concentrated under vacuum. The residue was purified by flash chromatography on silica gel eluting with dichloromethane/methanol (10:1) to afford 9-bromo-8-chloro-5,6-dihydro-4H-[1,4]oxazepino[5,6,7-de]quinazoline (2.80 g, 9.31 mmol, 51.2% yield) as a yellow solid. LC-MS: (ESI, m/z): 300.5 [M+H]⁺.

Step 3: 8-chloro-9-(4,4,5,5-tetramethyl-1,3,2-dioxaborolan-2-yl)-5,6-dihydro-4H-[1,4]oxazepino[5,6,7-de]quinazoline

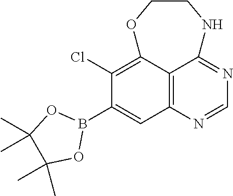 [SMILES: CC1(C)OB(c2cc3ncnc4c3c(c2Cl)OCCN4)OC1(C)C]

Under nitrogen, a solution of 9-bromo-8-chloro-5,6-dihydro-4H-[1,4]oxazepino[5,6,7-de]quinazoline (1.0 g, 3.33 mmol) 1,1′-Bis(diphenylphosphino)ferrocene-palladium(II)dichloride dichloromethane complex (271.7 mg, 0.33 mmol), potassium acetate (65.3 mg, 0.67 mmol) and Bis(pinacolato)diboron (2.53 g, 9.98 mmol) in 1,4-dioxane (25 mL) was stirred at 100° C. for 1.5 hours. After completion, the solvent was concentrated under vacuum. The reaction mixture was diluted with dichloromethane. After filtration, the reaction mixture was concentrated under vacuum to afford crude product that would be directly used in the next step without purification. LC-MS: (ESI, m/z): 347.6 [M+H]⁺.

Step 4: 6-(8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

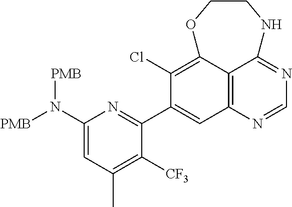

Under nitrogen, a solution of 8-chloro-9-(4,4,5,5-tetramethyl-1,3,2-dioxaborolan-2-yl)-5,6-dihydro-4H-[1,4]oxazepino[5,6,7-de]quinazoline (3.00 g, crude), potassium fluoride (703.6 mg, 12.12 mmol), Bis(triphenylphosphine)palladium(II) chloride (283.4 mg, 0.40 mmol) and 6-bromo-N,N-bis[(4-methoxyphenyl)methyl]-4-methyl-5-(trifluoromethyl)pyridin-2-amine (2.00 g, 4.04 mmol) in acetonitrile (25 mL) and water (5 mL) was stirred at 80° C. for 3 hours. After completion, the reaction mixture was diluted with ethyl acetate, wash with water, dried over anhydrous sodium sulfate and concentrated under vacuum. The residue was purified by flash chromatography on silica gel eluting with dichloromethane/methanol (10:1) to afford 6-(8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (1.04 g, 0.81 mmol, 40.5% yield) as a yellow solid. LC-MS: (ESI, m/z): 636.0 [M+H]⁺.

### Intermediate 6A: (S)-2-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-5-yl)acetonitrile

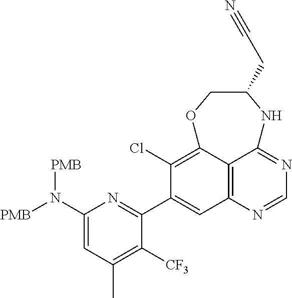

### Synthetic Route

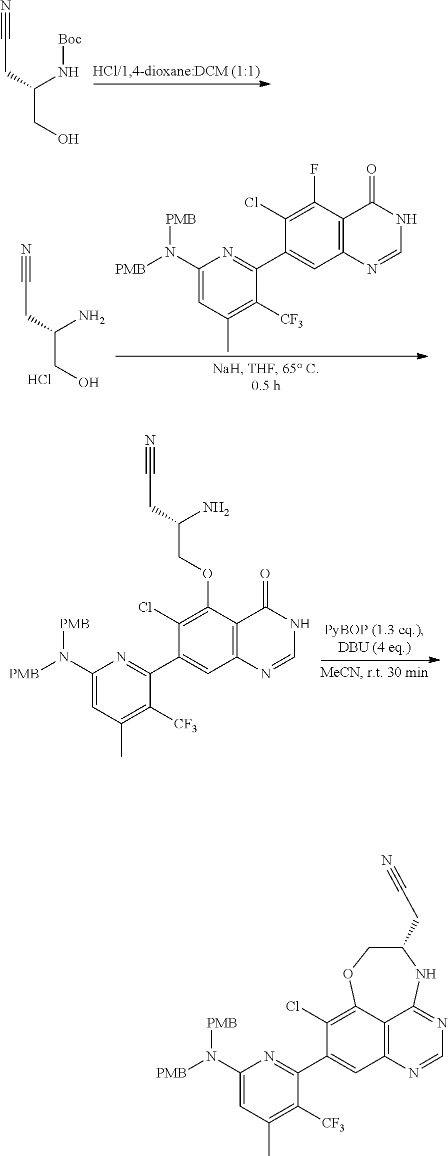

Step 1: (S)-3-amino-4-hydroxybutanenitrile hydrochloride

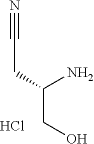

A solution of tert-butyl (S)-(1-cyano-3-hydroxypropan-2-yl)carbamate (2.30 g, 11.49 mmol) in hydrochloric acid (20.0 mL, 1 M in 1,4-dioxane) and dichloromethane (5.0 mL) was stirred at 25° C. for 4 hours. After completion, the solvent was concentrated under vacuum. The crude product would be directly used in the next step without purification. LC-MS: (ESI, m/z): 101.1 [M+H]⁺.

Step 2: (S)-3-amino-4-((7-(6-(bis(4-methoxybenzyl)amino)-4-methyl-3-(trifluoromethyl)pyridin-2-yl)-6-chloro-4-oxo-3,4-dihydroquinazolin-5-yl)oxy)butanenitrile

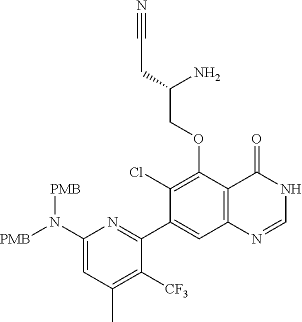

A solution of (S)-3-amino-4-hydroxybutanenitrile hydrochloride (2.5 g, crude) in tetrahydrofuran (25 mL) was added sodium hydride (812.5 mg, 20.3 mmol, 60% purity) at 0° C. Then 7-(6-(bis(4-methoxybenzyl)amino)-4-methyl-3-(trifluoromethyl)pyridin-2-yl)-6-chloro-5-fluoroquinazolin-4(3H)-one (2.5 g, 4.07 mmol) was added and stirred at 0° C. for 5 minutes. The resulting solution was stirred for 2 hours at 65° C. After completion, the residue was diluted with dichloromethane and the pH was adjusted to 7-8 with 2N hydrochloric acid. The solvent was concentrated under vacuum. The residue was purified by flash chromatography on silica gel eluting with dichloromethane/methanol (10/1) to afford (S)-3-amino-4-((7-(6-(bis(4-methoxybenzyl)amino)-4-methyl-3-(trifluoromethyl) pyridin-2-yl)-6-chloro-4-oxo-3,4-dihydroquinazolin-5-yl)oxy)butanenitrile (1.4 g, 2.02 mmol, 49.6% yield) as a yellow solid. LC-MS: (ESI, m/z): 693.2 [M+H]⁺.

Step 3: (S)-2-(9-(6-(bis(4-methoxybenzyl)amino)-4-methyl-3-(trifluoromethyl) pyridin-2-yl)-8-chloro-5,6-dihydro-4H-[1,4]oxazepino[5,6,7-de]quinazolin-5-yl)acetonitrile

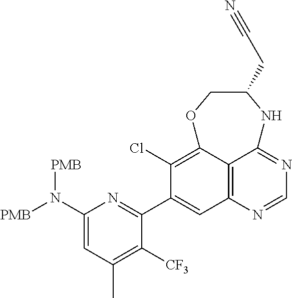

A solution of (S)-3-amino-4-((7-(6-(bis(4-methoxybenzyl)amino)-4-methyl-3-(trifluoromethyl)pyridin-2-yl)-6-chloro-4-oxo-3,4-dihydroquinazolin-5-yl)oxy)butanenitrile (1.4 g, 2.02 mmol), 1,8-diazabicyclo[5.4.0]undec-7-ene (1.54 g, 10.10 mmol) and benzotriazole-1-yl-oxytripyrrolidinophosphonium hexafluorophosphate (1.58 g, 3.03 mmol) in acetonitrile (14.0 mL) was stirred at 25° C. for 0.5 hours. After completion, the reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10:1) to afford (S)-2-(9-(6-(bis(4-methoxybenzyl)amino)-4-methyl-3-(trifluoromethyl) pyridin-2-yl)-8-chloro-5,6-dihydro-4H-[1,4]oxazepino[5,6,7-de]quinazolin-5-yl)acetonitrile (1 g, 1.48 mmol, 73.3% yield) as a yellow solid. LC-MS: (ESI, m/z): 675.2 [M+H]⁺.

### Intermediate 7A: (S)-(dihydro-1H,3H-spiro[pyrrolizine-2,2′-[1,3]dioxolan]-7a(5H)-yl)methanol

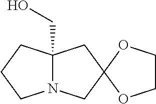 [SMILES: OC[C@@]12CCCN1CC1(C2)OCCO1]

### Synthetic Route

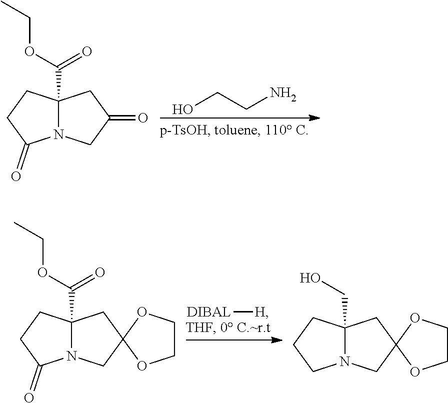

Step 1: ethyl (S)-5-oxodihydro-1H,3H-spiro[pyrrolizine-2,2′-[1,3]dioxolane]-7a(5H)-carboxylate

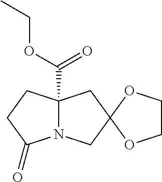 [SMILES: CCOC(=O)[C@@]12CCC(=O)N1CC1(C2)OCCO1]

A solution of ethyl (S)-2,5-dioxotetrahydro-1H-pyrrolizine-7a(5H)-carboxylate (1.00 g, 4.73 mmol), ethylene glycol (450.1 mg, 7.25 mmol) and p-toluenesulfonic acid (158.0 mg, 0.92 mmol) in toluene (50 mL) was stirred at 110° C. for 1 hour. After completion, the reaction was concentrated under vacuum, diluted with dichloromethane, washed with water and the organic layer was combined. The organic layer was dried over anhydrous sodium sulfate and concentrated under vacuum to afford ethyl (S)-5-oxodihydro-1H,3H-spiro[pyrrolizine-2,2′-[1,3]dioxolane]-7a(5H)-carboxylate (1.17 g, 4.58 mmol, 96.8% yield) as a brown oil. LC-MS: (ESI, m/z): 256.1 [M+H]⁺

Step 2: (S)-(dihydro-1H,3H-spiro[pyrrolizine-2,2′-[1,3]dioxolan]-7a(5H)-yl)methanol

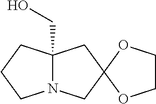 [SMILES: OC[C@@]12CCCN1CC1(C2)OCCO1]

Under nitrogen, a solution of ethyl (S)-5-oxodihydro-1H,3H-spiro[pyrrolizine-2,2′-[1,3]dioxolane]-7a(5H)-carboxylate (700.0 mg, 2.74 mmol) in tetrahydrofuran (35 mL) was added diisobutylaluminium hydride (8.23 mL, 8.23 mmol, 1 M in toluene) at 0° C. and stirred for 30 minutes at room temperature. After completion, the reaction was quenched with ammonium chloride solution, diluted with dichloromethane, washed with water and the organic layer was combined. The aqueous phase was concentrated under vacuum to afford (S)-(dihydro-1H,3H-spiro[pyrrolizine-2,2′-[1,3]dioxolan]-7a(5H)-yl)methanol (180.1 mg, crude) as a yellow oil. LC-MS: (ESI, m/z): 200.1 [M+H]⁺

### Intermediate 8A: 9-bromo-8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline

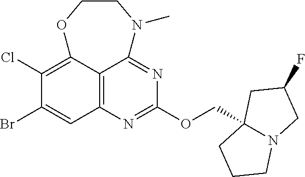 [SMILES: CN1CCOc2c(Cl)c(Br)cc3nc(OC[C@@]45CCCN4C[C@H](F)C5)nc1c23]

### Synthetic Route

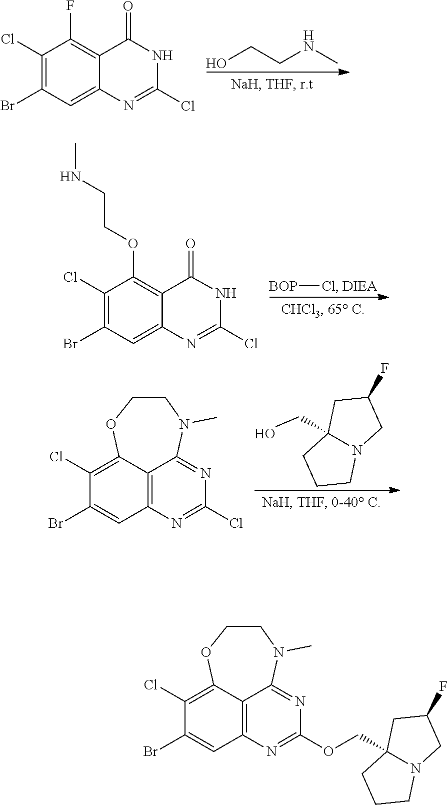

Step 1:7-bromo-2,6-dichloro-5-(2-(methylamino)ethoxy)quinazolin-4(3H)-one

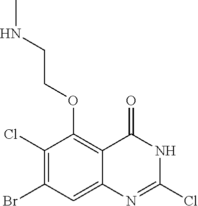 [SMILES: CNCCOc1c(Cl)c(Br)cc2nc(Cl)[nH]c(=O)c12]

To a solution of 2-(methylamino)ethanol (1.32 g, 17.63 mmol) in tetrahydrofuran (50 mL) was added sodium hydride (1.92 g, 48.09 mmol), the mixture was stirred at 0° C. for 1 hour. Then 7-bromo-2,6-dichloro-5-fluoro-3H-quinazolin-4-one (5.00 g, 16.03 mmol) was added and stirred at 25° C. for 1 hour. After completion, the reaction was quenched with 1N hydrochloric acid solution. After filtration, the solids were collected to afford 7-bromo-2,6-dichloro-5-(2-(methylamino)ethoxy)quinazolin-4(3H)-one (8.40 g crude) as a brown solid. LC-MS: (ESI, m/z): 366.0 [M+H]⁺

Step 2: 9-bromo-2,8-dichloro-4-methyl-5,6-dihydro-4H-[1,4]oxazepino[5,6,7-de]quinazoline

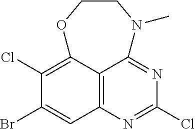 [SMILES: CN1CCOc2c(Cl)c(Br)cc3nc(Cl)nc1c23]

A mixture of 7-bromo-2,6-dichloro-5-[2-(methylamino)ethoxy]-3H-quinazolin-4-one (8.30 g, 22.61 mmol), N,N-diisopropylethylamine (5.84 g, 45.23 mmol) and Bis(2-oxo-3-oxazolidinyl)phosphinic chloride (6.89 g, 27.14 mmol) in chloroform (80 mL) was stirred at 65° C. for 1 hour. The resulting solution was diluted with water and extracted with Ethyl acetate. The organic layers were washed with brine, dried over anhydrous Sodium sulfate and concentrated. The residue was purified by flash chromatography on silica gel eluting with ethyl acetate/petroleum ether (1/1) to afford 9-bromo-2,8-dichloro-4-methyl-5,6-dihydro-4H-[1,4]oxazepino[5,6,7-de]quinazoline (3.74 g, 9.64 mmol, 42.6% yield) as a yellow solid. LC-MS: (ESI, m/z): 348.0 [M+H]⁺

Step 3: 9-bromo-8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline

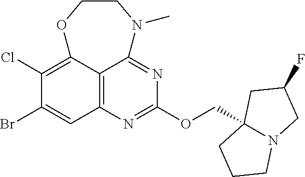 [SMILES: CN1CCOc2c(Cl)c(Br)cc3nc(OC[C@@]45CCCN4C[C@H](F)C5)nc1c23]

To a solution of [rac-(2R,8S)-2-fluoro-1,2,3,5,6,7-hexahydropyrrolizin-8-yl]methanol (638.6 mg, 4.0 mmol) in tetrahydrofuran (10 mL) was added sodium hydride (343.8 mg, 8.6 mmol), the mixture was stirred at 0° C. for 0.5 hour. Then 9-bromo-2,8-dichloro-4-methyl-5,6-dihydro-4H-[1,4]oxazepino[5,6,7-de]quinazoline (1.00 g, 2.87 mmol) was added and stirred at 40° C. for 1 hour. After completion, the reaction was quenched with 1N hydrochloric acid solution. The resulting solution was diluted with water and extracted with Ethyl acetate. The organic layers were washed with brine, dried over anhydrous Sodium sulfate and concentrated. The residue was purified by flash chromatography on silica gel eluting with methanol/dichloromethane (1/25) to afford 9-bromo-8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline (1.10 g, 2.19 mmol, 76.5% yield) as a yellow solid. LC-MS: (ESI, m/z): 471.1 [M+H]⁺

Example 1: 6-(4-((1H-pyrazol-5-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

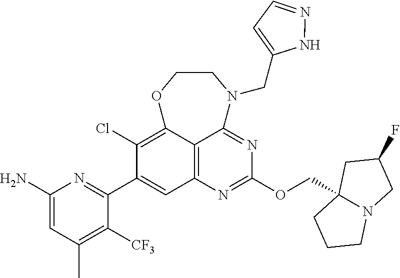 [SMILES: Cc1cc(N)nc(-c2cc3nc(OC[C@@]45CCCN4C[C@H](F)C5)nc4c3c(c2Cl)OCCN4Cc2ccn[nH]2)c1C(F)(F)F]

### Synthetic Route

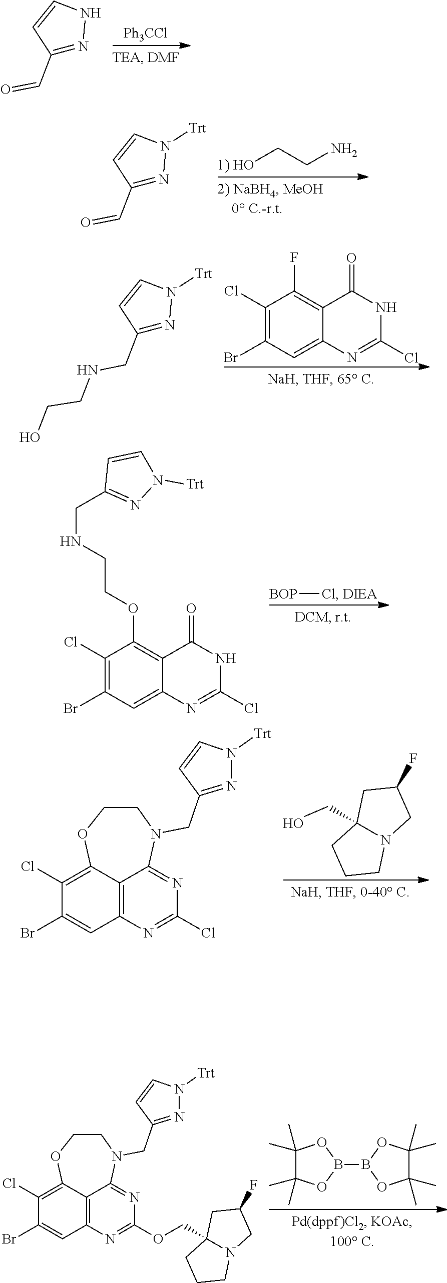

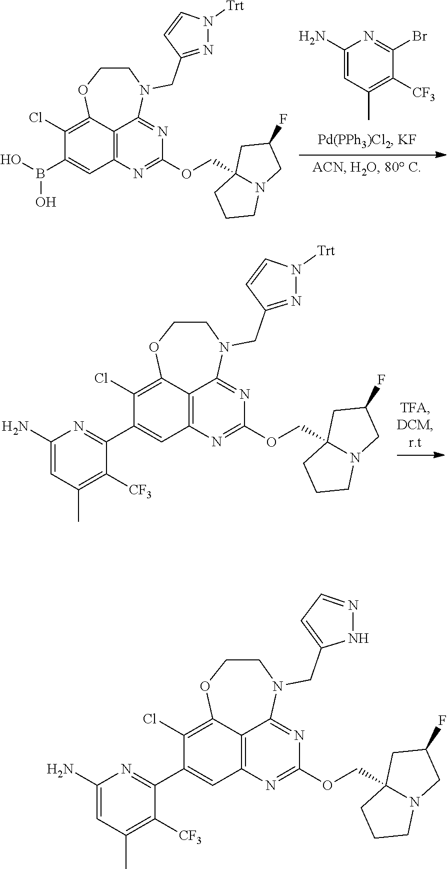

Step 1: 1-trityl-1H-pyrazole-3-carbaldehyde

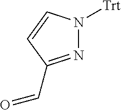

A solution of 1H-pyrazole-3-carbaldehyde (4.00 g, 41.62 mmol), tritylchloride (17.41 g, 62.40 mmol) and triethylamine (17.4 mL, 124.96 mmol) in N,N-dimethylformamide (40 mL) was stirred at 25° C. for 8 hours. After completion, the reaction mixture was diluted with water. The resulting solution was extracted with dichloromethane and the organic layers were combined. The organic layers were washed with water again. The organic layer was dried over anhydrous sodium sulfate and concentrated under vacuum. The residue was purified by flash chromatography on silica gel eluting with ethyl acetate/petroleum ether (1/30) to afford 1-tritylpyrazole-3-carbaldehyde (11.00 g, 32.55 mmol) as a white solid. LC-MS: (ESI, m/z): 339.1 [M+H]⁺

Step 2: 2-(((1-trityl-1H-pyrazol-3-yl)methyl)amino)ethan-1-ol

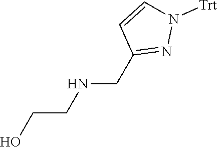

A solution of 1-tritylpyrazole-3-carbaldehyde (6.00 g, 17.70 mmol), 2-aminoethanol (3.2 mL, 53.20 mmol) and acetic acid (0.11 g, 1.87 mmol) in methyl alcohol (50 mL) was stirred at 25° C. for 3 hours. Then sodium cyanoborohydride (2.23 g, 35.53 mmol) was added and stirred at 25° C. for 4 hours. After completion, the reaction was quenched with water. The reaction mixture was concentrated under reduced pressure. The residue was purified by flash chromatography on silica gel eluting with methanol/dichloromethane (1/20) to afford 2-[(1-tritylpyrazol-3-yl)methylamino]ethanol (1.20 g, 3.12 mmol) as a colorless oil. LC-MS: (ESI, m/z): 384.2 [M+H]⁺

Step 3: 7-bromo-2,6-dichloro-5-(2-(((1-trityl-1H-pyrazol-3-yl)methyl)amino)ethoxy)quinazolin-4(3H)-one

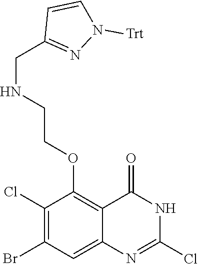

A solution of 2-[(1-tritylpyrazol-3-yl)methylamino]ethanol (1.12 g, 2.9 mmol) and sodium hydride (292.4 mg, 7.3 mmol) in tetrahydrofuran (10 mL) was stirred at 0° C. for 15 minutes. Then 7-bromo-2, 6-dichloro-5-fluoro-3H-quinazolin-4-one (760.0 mg, 2.4 mmol) was added and stirred at 65° C. for 1 hour. After completion, the reaction mixture was adjusted to pH=6 with hydrochloric acid (1N). The solution was concentrated under vacuum. The residue was purified by flash chromatography on silica gel eluting with methanol/dichloromethane (1/20) to afford 7-bromo-2,6-dichloro-5-(2-(((1-trityl-1H-pyrazol-3-yl)methyl)amino)ethoxy)quinazolin-4(3H)-one

(1.60 g, 2.37 mmol) as a white solid. LC-MS: (ESI, m/z): 674.1 [M+H]⁺

Step 4: 9-bromo-2, 8-dichloro-4-((1-trityl-1H-pyrazol-3-yl)methyl)-5,6-dihydro-4H-[1,4]oxazepino[5,6,7-de]quinazoline

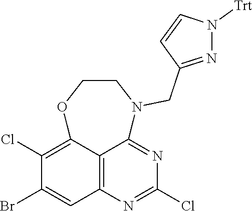

A solution of 7-bromo-2,6-dichloro-5-(2-(((1-trityl-1H-pyrazol-3-yl)methyl)amino)ethoxy)quinazolin-4(3H)-one (1.60 g, 2.37 mmol), bis(2-oxo-3-oxazolidinyl)phosphinic chloride (0.90 g, 3.55 mmol) and N,N-diisopropylethylamine (0.61 g, 4.75 mmol) in chloroform (15 mL) was stirred at 70° C. for 1 hour. After completion, the reaction mixture was diluted with dichloromethane. The resulting solution was washed with water. The organic layer was dried over anhydrous sodium sulfate and concentrated under vacuum. The residue was purified by flash chromatography on silica gel eluting with methanol/dichloromethane (1/50) to afford 7-bromo-3,8-dichloro-13-[(1-tritylpyrazol-3-yl)methyl]-10-oxa-2,4,13-triazatricyclo[7.4.1.05,14]tetradeca-1,3,5(14),6,8-pentaene (700.0 mg, 1.06 mmol) as a white solid. LC-MS: (ESI, m/z): 656.1 [M+H]⁺

Step 5: 9-bromo-8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazoline

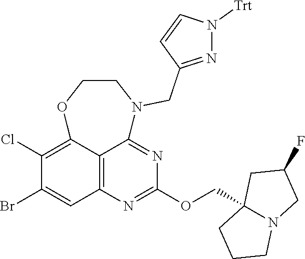

A solution of ((2R,7aS)-2-fluorotetrahydro-1H-pyrrolizin-7a(5H)-yl)methanol(181.6 mg, 1.12 mmol) and sodium hydride (152.1 mg, 3.81 mmol) in tetrahydrofuran (5 mL) was stirred at 0° C. for 15 minutes. Then 7-bromo-3,8-dichloro-13-[(1-tritylpyrazol-3-yl)methyl]-10-oxa-2,4,13-tiazatricyclo[7.4.1.05,14]tetradeca-1,3,5(14),6,8-pentaene (500.0 mg, 0.81 mmol) was added and stirred at 40° C. for 5 hours. After completion, the reaction mixture was adjusted to pH=6 with saturated ammonium chloride solution. The reaction mixture was diluted with ethyl acetate (60 mL). The resulting solution was washed with water. The organic layer was dried over anhydrous sodium sulfate and concentrated under vacuum. The residue was purified by flash chromatography on silica gel eluting with ethyl acetate/petroleum ether (1/8) to afford 9-bromo-8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazoline (400.0 mg, 0.51 mmol) as a white solid. LC-MS: (ESI, m/z): 779.3 [M+H]⁺

Step 6: (8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazolin-9-yl)boronic acid

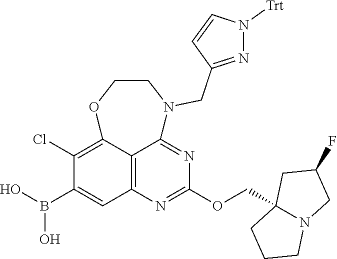

Under nitrogen, a solution of 9-bromo-8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazoline (380.0 mg, 0.51 mmol), bis(pinacolato)diboron (247.4 mg, 0.92 mmol), [1,1′-bis(diphenylphosphino)ferrocene]dichloropalladium(II) (35.6 mg, 0.051 mmol) and potassium acetate (95.6 mg, 0.92 mmol) in 1,4-dioxane (3 mL) was stirred at 100° C. for 2 hours. After completion, the reaction mixture was concentrated under reduced pressure. And then the reaction mixture was diluted with dichloromethane. After filtration, the organic was collected and concentrated under vacuum. The crude product (8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazolin-9-yl)boronic acid (600.0 mg, crude) was used in the next step directly without further purification. LC-MS: (ESI, m/z): 745.3 [M+H]⁺

Step 7: 6-(8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazolin-9-yl)-4-methyl-5-(trifluoromethyl)pyridin-2-amine

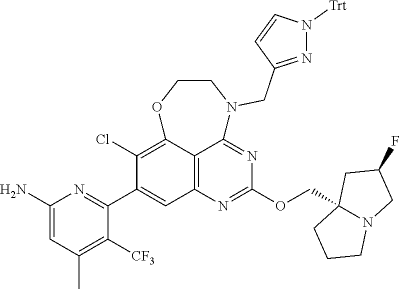

Under nitrogen, a solution of (8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazolin-9-yl)boronic acid (597.4 mg, crude), 6-bromo-4-methyl-5(trifluoromethyl)pyridin-2-amine (170.4 mg, 0.73 mmol), bis(triphenylphosphine)palladium(II) chloride (46.9 mg, 0.073 mmol) and sodium carbonate (141.6 mg, 1.36 mmol) in acetonitrile (4 mL) and water (1 mL) was stirred at 80° C. for 1 hour. After completion, the reaction mixture was concentrated under reduced pressure. Then the reaction mixture was diluted with dichloromethane. After filtration, the organic phase was collected and concentrated under vacuum. The residue was purified by flash chromatography on silica gel eluting with methanol/dichloromethane (1/20) to afford 6-(8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazolin-9-yl)-4-methyl-5-(trifluoromethyl)pyridin-2-amine (250.0 mg, 0.28 mmol) as a brown solid. LC-MS: (ESI, m/z): 875.4 [M+H]⁺

Step 8: 6-(4-((1H-pyrazol-5-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

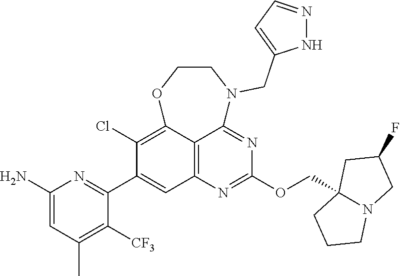 [SMILES: Cc1cc(N)nc(-c2cc3nc(OC[C@@]45CCCN4C[C@H](F)C5)nc4c3c(c2Cl)OCCN4Cc2ccn[nH]2)c1C(F)(F)F]

A solution of 6-(8-chloro-2-(((2R,7aS)-2-fluorotetrahydro-1H-pyrrolizin-7a(5H)-yl)methoxy)-4-((1-trityl-1H-pyrazol-3-yl)methyl)-5,6-dihydro-4H-[1,4]oxazepino[5,6,7-de]quinazolin-9-yl)-4-methyl-5-(trifluoromethyl)pyridin-2-amine (150.0 mg, 0.171 mmol) in 2,2,2-trifluoroacetic acid (0.5 mL) and dichloromethane (0.5 mL) was stirred at 25° C. for 2 hours. After completion, the reaction mixture was concentrated under reduced pressure. The crude product was purified by Prep-HPLC with the following conditions (Column: XBridge Prep OBD C18 Column, 30×150 mm 5 um: Mobile Phase A: Water (10 MMOL/L NH4HCO3), Mobile Phase B: ACN; Flow rate: 60 mL/min; Gradient: 39 B to 49 B in 8 min, 254/220 nm; RT1: 7.6; RT2;) to afford 6-(4-((1H-pyrazol-5-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (29.7 mg, 0.059 mmol, 34.5% yield). LC-MS: (ESI, m/z): 633.3 [M+H]⁺

Example 1:1H NMR (300 MHz, DMSO-d₆) δ 12.65 (s, 1H), 7.64 (s, 1H), 6.93 (s, 1H), 6.75 (s, 2H), 6.44 (s, 1H), 6.25 (d, J=2.2 Hz, 1H), 5.27 (d, J=54.1 Hz, 1H), 5.13-4.93 (m, 2H), 4.68-4.43 (m, 2H), 4.16-3.87 (m, 4H), 3.10 (s, 2H), 3.00 (s, 1H), 2.90-2.72 (m, 1H), 2.35 (d, J=2.3 Hz, 3H), 2.19-2.09 (m, 1H), 2.06-1.92 (m, 2H), 1.91-1.64 (m, 3H).

Example 2: 6-(4-((1H-pyrazol-4-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

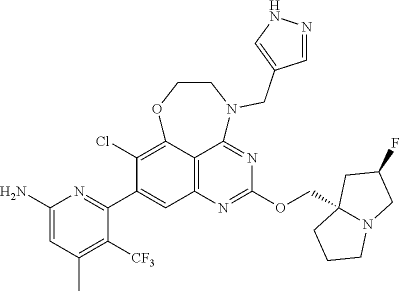 [SMILES: Cc1cc(N)nc(-c2cc3nc(OC[C@@]45CCCN4C[C@H](F)C5)nc4c3c(c2Cl)OCCN4Cc2cn[nH]c2)c1C(F)(F)F]

### Synthetic Route

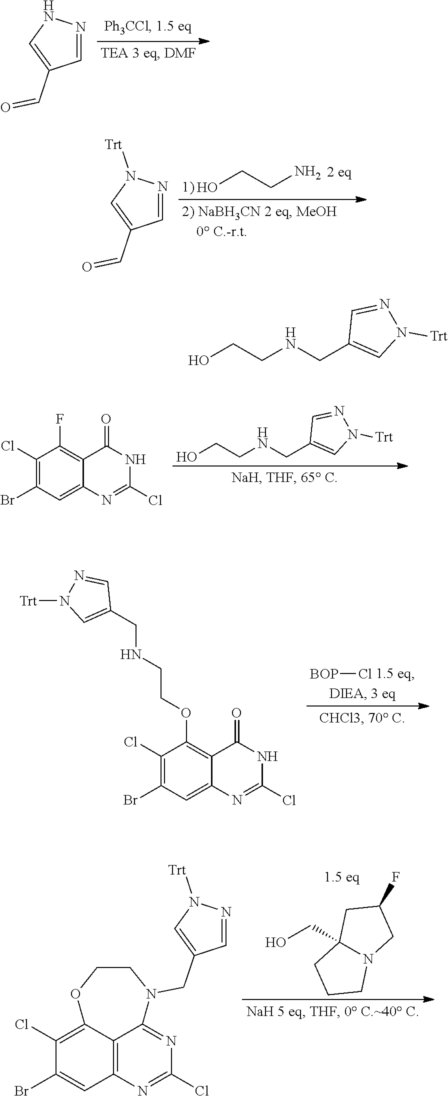

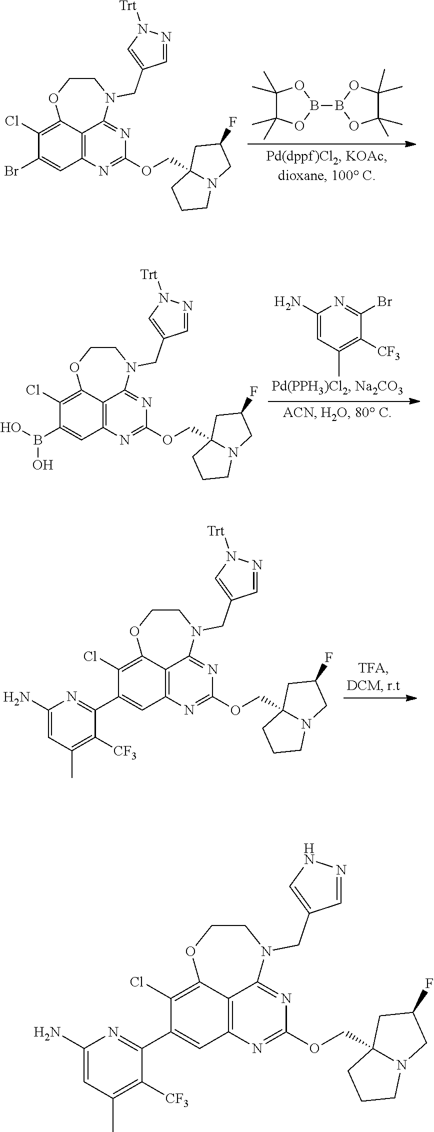

Step 1: 1-trityl-1H-pyrazole-4-carbaldehyde

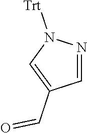

A solution of 1H-pyrazole-4-carbaldehyde (3.00 g, 31.22 mmol), triethylamine (13 mL, 93.66 mmol) and tritylchloride (13.10 g, 46.84 mmol) in N,N-dimethylformamide (30 mL) was stirred at 25° C. for 4 hours. After reaction completion, the solvent was concentrated under vacuum. The residue was purified by flash chromatography on silica gel eluting with petroleum ether/ethyl acetate (10/1) to afford 1-trityl-1H-pyrazole-4-carbaldehyde (2.40 g, 7.07 mmol) as a white solid. LC-MS: (ESI, m/z): 339.4 [M+H]⁺

Step 2: 2-(((1-trityl-1H-pyrazol-4-yl)methyl)amino)ethan-1-ol

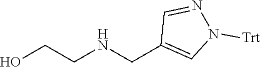

A solution of 1-tritylpyrazole-4-carbaldehyde (1.50 g, 4.42 mmol), 2-aminoethanol (0.54 mL, 8.84 mmol) and acetic acid (0.03 mL, 0.03 mmol) in methyl alcohol (1 mL) was stirred at 25° C. for 2 hours. Then sodium cyanoborohydride (0.56 g, 8.84 mmol) was added and stirred at 25° C. for 2 hours. After reaction completion, the solvent was quenched with water and concentrated under vacuum. The residue was purified by flash chromatography on silica gel eluting with dichloromethane/methanol (10/1) to afford 2-(((1-trityl-1H-pyrazol-4-yl)methyl)amino)ethan-1-ol (630.0 mg, 1.64 mmol) as a white solid. LC-MS: (ESI, m/z): 384.4 [M+H]⁺

Step 3: 7-bromo-2, 6-dichloro-5-(2-(((1-trityl-1H-pyrazol-4-yl)methyl)amino)ethoxy)quinazolin-4(3H)-one

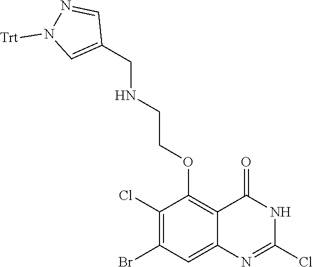

A solution 2-(((1-trityl-1H-pyrazol-4-yl)methyl)amino)ethan-1-ol (991.4 mg, 2.58 mmol) and sodium hydride (258.5 mg, 6.45 mmol, 60% purity) in tetrahydrofuran (10 mL) was stirred at 0° C. for 5 minutes. Then 7-bromo-2, 6-dichloro-5-fluoro-3H-quinazolin-4-one (10.0 mg, 0.03 mmol) was added and stirred at 65° C. for 4 hours. The solvent was quenched with 1 M hydrochloric acid and concentrated under vacuum. The residue was purified by flash chromatography on silica gel eluting with dichloromethane/methanol (10/1) to afford 7-bromo-2,6-dichloro-5-(2-(((1-trityl-1H-pyrazol-4-yl)methyl)amino)ethoxy)quinazolin-4(3H)-one (550 mg, 0.81 mmol) as a white solid. LC-MS: (ESI, m/z): 674.4 [M+H]⁺

Step 4: 9-bromo-2,8-dichloro-4-((1-trityl-1H-pyrazol-4-yl)methyl)-5,6-dihydro-4H-[1,4]oxazepino[5,6,7-de]quinazoline

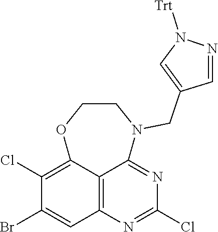

A solution of 7-bromo-2,6-dichloro-5-(2-(((1-trityl-1H-pyrazol-4-yl)methyl)amino)ethoxy)quinazolin-4(3H)-one (1.10 g, 1.53 mmol), bis(2-oxo-3-oxazolidinyl)phosphinic chloride (0.57 g, 2.24 mmol) and N,N-diisopropylethylamine (0.5 mL, 2.99 mmol) in chloroform (100 mL) was stirred at 70° C. for 4 hours. After reaction completion, the solvent was concentrated under vacuum. The residue was purified by flash chromatography on silica gel eluting with methanol/dichloromethane (1/10) to afford 9-bromo-2,8-dichloro-4-((1-trityl-1H-pyrazol-4-yl)methyl)-5,6-dihydro-4H-[1,4]oxazepino[5,6,7-de]quinazoline (300.0 mg, 0.45 mmol) as a white solid. LC-MS: (ESI, m/z): 656.1 [M+H]⁺

Step 5: 9-bromo-8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazoline

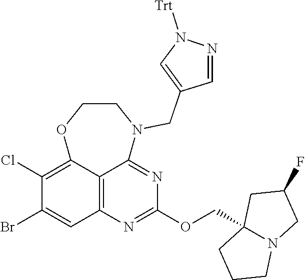

A solution of ((2R,7aS)-2-fluorotetrahydro-1H-pyrrolizin-7a(5H)-yl)methanol (108.9 mg, 0.6 mmol) and sodium hydride (91.2 mg, 2.3 mmol, 60% purity) in tetrahydrofuran (4 mL) was stirred at 0° C. for 10 minutes. Then 7-bromo-3,8-dichloro-13-[(1-tritylpyrazol-4-yl)methyl]-10-oxa-2,4,13-triazatricyclo[7.4.1.05,14]tetradeca-1,3,5(14),6,8-pentaene (300.0 mg, 0.4 mmol) was added and stirred at 40° C. for 2 hours. After completion, the reaction was quenched by dilute hydrochloric acid. The solvent was concentrated under vacuum. The residue was purified by flash chromatography on silica gel eluting with methanol/dichloromethane (1/50) to afford 9-bromo-8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazoline (200.0 mg, 0.25 mmol) as a white solid. LC-MS: (ESI, m/z): 779.2 [M+H]⁺

Step 6: (8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazolin-9-yl)boronic acid

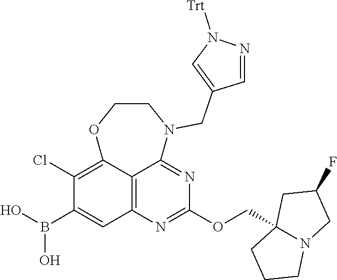

Under nitrogen, a solution of 9-bromo-8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazoline (165.0 mg, 0.23 mmol), bis(pinacolato)diboron (107.4 mg, 0.46 mmol), [1,1′-bis(diphenylphosphino)ferrocene]dichloropalladium(II) (15.48 mg, 0.02 mmol) and potassium acetate (0.03 mL, 0.46 mmol) in 1,4-dioxane (3 mL) was stirred at 100° C. for 1.5 hours. After completion, the reaction mixture was concentrated under reduced pressure. And then the reaction mixture was diluted with dichloromethane (20 mL). After filtration, the organic was collected and concentrated under vacuum. The crude product (8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazolin-9-yl)boronic acid (340.0 mg, crude) (brown oil) was used in the next step directly without further purification. LC-MS: (ESI, m/z): 745.4 [M+H]⁺

Step 7: 6-(8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazolin-9-yl)-4-methyl-5-(trifluoromethyl)pyridin-2-amine

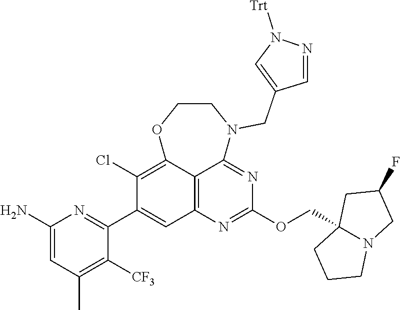

Under nitrogen, a solution of (8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazolin-9-yl)boronic acid (315.5 mg, crude), 6-bromo-4-methyl-5-(trifluoromethyl)pyridin-2-amine (90.0 mg, 0.46 mmol), bis(triphenylphosphine)palladium(II) chloride (24.8 mg, 0.04 mmol) and sodium carbonate (74.8 mg, 0.79 mmol) in acetonitrile (4 mL)/water (1 mL) was added and stirred at 80° C. for 1 hour. After completion, the reaction mixture was diluted with dichloromethane. The resulting solution was washed with water. The organic layer was dried over anhydrous sodium sulfate and concentrated under vacuum. The residue was purified by flash chromatography on silica gel eluting with methanol/dichloromethane (1/20) to afford 6-(8-chloro-2-(((2R,7aS)-2-fluorotetrahydro-1H-pyrrolizin-7a(5H)-yl)methoxy)-4-((1-trityl-1H-pyrazol-4-yl)methyl)-5,6-dihydro-4H-[1,4]oxazepino[5,6,7-de]quinazolin-9-yl)-4-methyl-5-(trifluoromethyl)pyridin-2-amine (130.0 mg, 0.15 mmol) as a brown solid. LC-MS: (ESI, m/z): 875.4 [M+H]⁺

Step 8: 6-(4-((1H-pyrazol-4-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

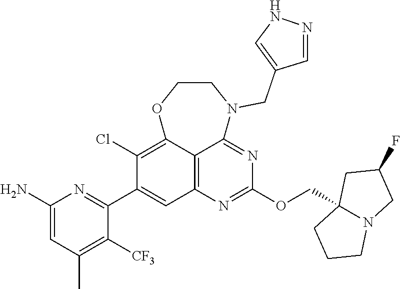 [SMILES: Cc1cc(N)nc(-c2cc3nc(OC[C@@]45CCCN4C[C@H](F)C5)nc4c3c(c2Cl)OCCN4Cc2cn[nH]c2)c1C(F)(F)F]

A solution of 6-(4-((1H-pyrazol-4-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (100.0 mg, 0.12 mmol) in 2,2,2-trifluoroacetic acid (0.5 mL)/dichloromethane (0.5 mL) was stirred at 25° C. for 2 hours. After completion, the reaction mixture was concentrated under reduced pressure. The crude product was purified by Prep-HPLC with the following conditions (Column: XBridge Prep OBD C18 Column, 30×150 mm 5 um; Mobile Phase A: Water (10 MMOL/L NH4HCO3), Mobile Phase B: ACN; Flow rate: 60 mL/min; Gradient: 37 B to 46 B in 9 min, 254/220 nm; RT1: 8.17; RT2;) to afford 6-(4-((1H-pyrazol-4-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (24.7 mg, 0.04 mmol, 33.3% yield). LC-MS: (ESI, m/z): 633.3 [M+H]⁺

Example 2: ¹H NMR (300 MHz, DMSO-d₆) δ 12.79 (s, 1H), 7.68 (s, 2H), 6.94 (s, 1H), 6.74 (s, 2H), 6.44 (s, 1H), 5.26 (d, J=53.7 Hz 1H), 4.96-4.76 (m, 2H), 4.64-4.40 (m, 2H), 4.18-3.95 (m, 2H), 3.95-3.78 (m, 2H), 3.16-2.91 (m, 3H), 2.89-2.72 (m, 1H), 2.34 (d, J=2.2 Hz, 3H), 2.20-2.08 (m, 1H), 2.08-1.90 (m, 2H), 1.90-1.62 (m, 3H).

Example 3: 6-(4-((5-aminopyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

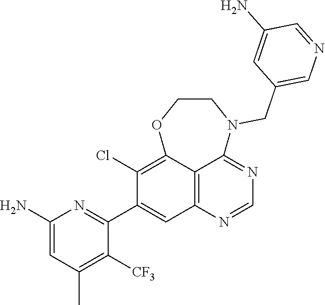 [SMILES: Cc1cc(N)nc(-c2cc3ncnc4c3c(c2Cl)OCCN4Cc2cncc(N)c2)c1C(F)(F)F]

### Synthetic Route

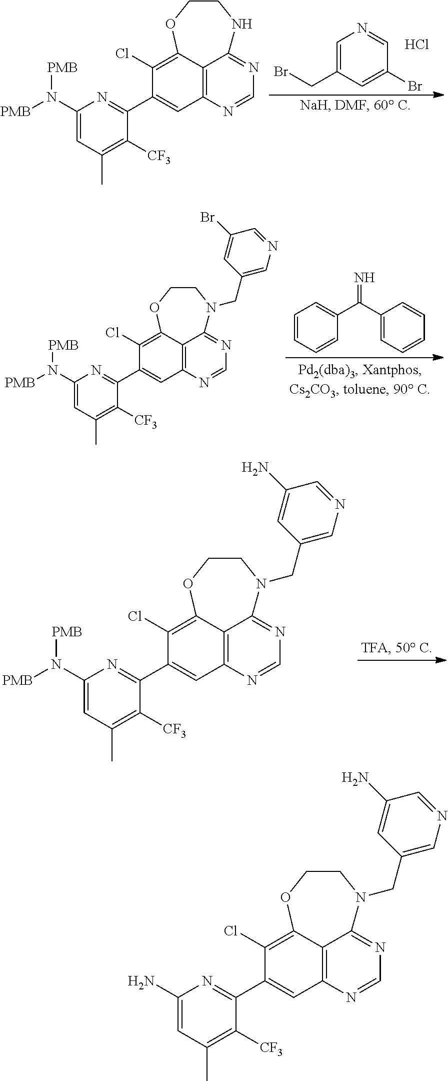

Step 1: 6-(4-((5-bromopyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

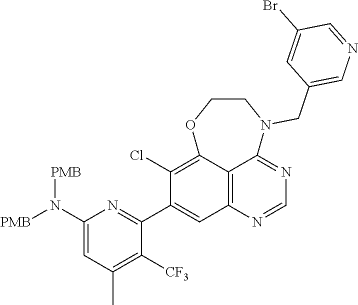

A solution of 3-bromo-5-(bromomethyl)pyridine (236.6 mg, 0.96 mmol) and sodium hydride (60% purity) (25.1 mg, 0.64 mmol) in N,N-dimethylacetamide (1 mL) was stirred at 25° C. for 10 min. Then 6-(8-chloro-10-oxa-2,4,13-triazatricyclo[7.4.1.05,14]tetradeca-1,3,5(14),6,8-pentaen-7-yl)-N,N-bis[(4-methoxyphenyl)methyl]-4-methyl-5-(trifluoromethyl)pyridin-2-amine (200.0 mg, 0.32 mmol) was added and stirred for 2 hours. After completion, the reaction mixture was quenched with saturated ammonium chloride solution, diluted with water and extracted with ethyl acetate. The combined organic layers were dried over anhydrous sodium sulfate and concentrated under vacuum to afford the crude product. The residue was purified by flash chromatography on silica gel eluting with dichloromethane/methanol (10/1) to afford 6-(4-((5-bromopyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (150.0 mg, 0.18 mmol, 51.5% yield) as a yellow solid. LC-MS: (ESI, m/z): 805.1 [M+H]⁺

Step 2: 6-(4-((5-aminopyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

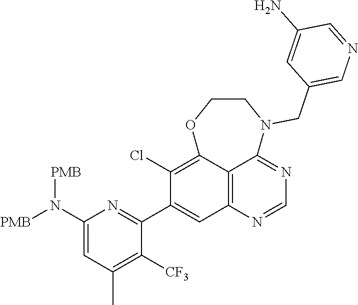

Under nitrogen, a solution of 6-(4-((5-bromopyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (150.0 mg, 0.18 mmol), tris(dibenzylideneacetone)dipalladium (17.0 mg, 0.02 mmol), 4,5-bis(diphenylphosphino)-9,9-dimethylxanthene (21.5 mg, 0.04 mmol), diphenylmethanimine (261.35 mg, 1.44 mmol) and cesium carbonate (121.2 mg, 0.36 mmol) in toluene (3 mL) was stirred for 16 h at 90° C. After completion, the reaction mixture was concentrated under reduced pressure and diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford 6-(4-((5-aminopyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (100.0 mg, 0.13 mmol, 73.8% yield) as a black solid. LC-MS: (ESI, m/z): 742.2 [M+H]⁺

Step 3: 6-(4-((5-aminopyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

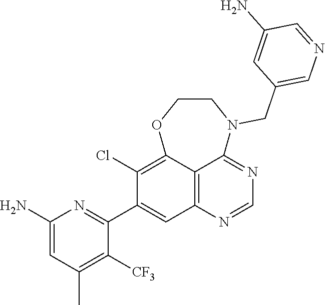 [SMILES: Cc1cc(N)nc(-c2cc3ncnc4c3c(c2Cl)OCCN4Cc2cncc(N)c2)c1C(F)(F)F]

A solution of 6-(4-((5-aminopyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (100.0 mg, 0.13 mmol) and trifluoroacetic acid (5 mL) was stirred at 50° C. for 1 hour. After completion, the reaction mixture was concentrated under reduced pressure to afford the crude product. The crude product was purified by Prep-HPLC with the following conditions: Column: XBridge Prep OBD C18 Column, 30×150 mm Sum; Mobile Phase A: Water (10 MMOL/L NH4HCO3), Mobile Phase B: ACN; Detector, UV 254 nm. RT: 8.5 to afford 6-(4-((5-aminopyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine (20.7 mg, 0.04 mmol, 30.3% yield). LC-MS: (ESI, m/z): 502.1 [M+H]⁺

Example 3: ¹H NMR (300 MHz, DMSO-d₆, ppm) δ 8.43 (s, 1H), 7.83 (d, J=2.6 Hz, 1H), 7.75 (d, J=1.9 Hz, 1H), 7.20 (s, 1H), 6.85 (t, J=2.3 Hz, 1H), 6.77 (s, 2H), 6.46 (s, 1H), 5.30 (brs, 2H), 5.11-4.93 (m, 2H), 4.61 (q, J=3.4 Hz, 2H), 3.95-3.86 (m, 2H), 2.36 (d, J=2.3 Hz, 3H).

Example 4: (S)-6-(4-(1-(5-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

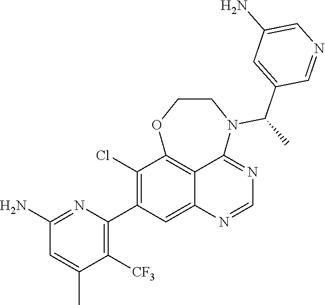 [SMILES: Cc1cc(N)nc(-c2cc3ncnc4c3c(c2Cl)OCCN4[C@@H](C)c2cncc(N)c2)c1C(F)(F)F]

### Synthetic Route

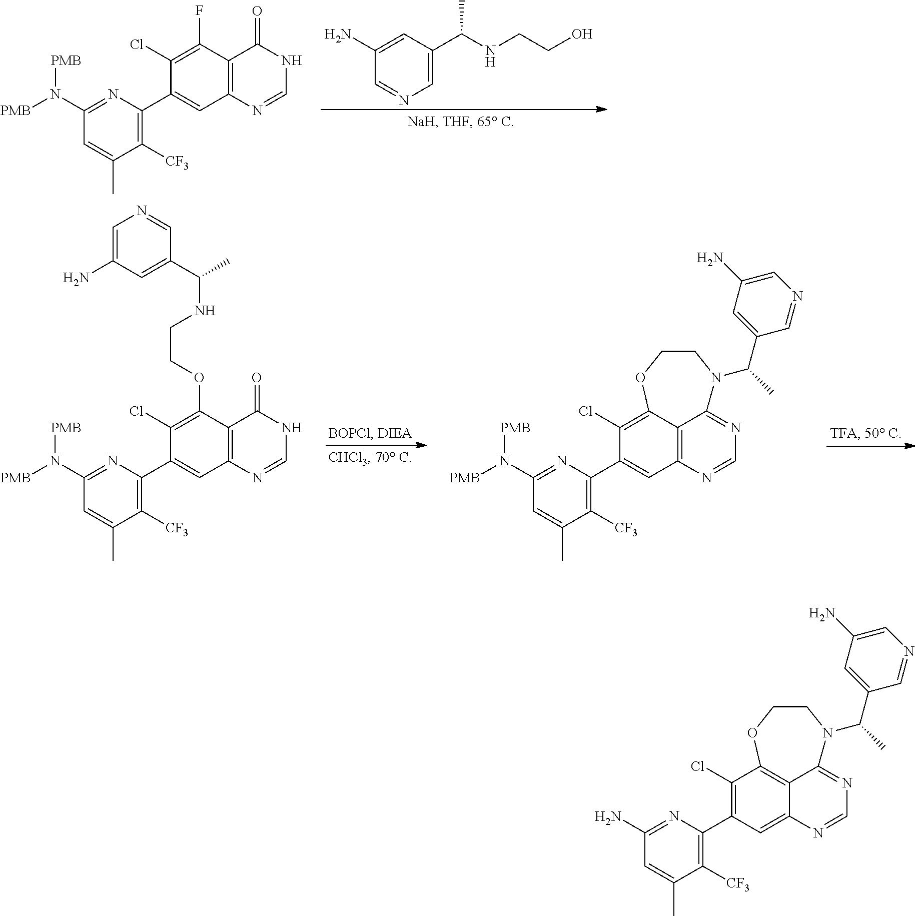

Step 1: (S)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one

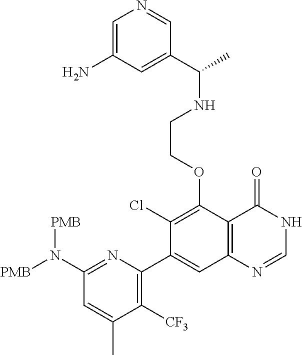

A solution of 2-[[rac-(1S)-1-(5-amino-3-pyridyl)ethyl]amino]ethanol (133.0 mg, 0.73 mmol) and sodium hydride (60% purity) (46.9 mg, 1.94 mmol) in tetrachloroethylene (5 mL) was stirred at 0° C. for 20 minutes. Then 7-(6-(bis(4-methoxybenzyl)amino)-4-methyl-3-(trifluoromethyl)pyridin-2-yl)-6-chloro-5-fluoroquinazolin-4(3H)-one (300.0 mg, 0.49 mmol) was added and stirred at 65° C. for 1 hour. After completion, the reaction mixture was quenched with saturated ammonium chloride, diluted with water and extracted with ethyl acetate. The combined organic layers were dried over anhydrous sodium sulfate and concentrated under vacuum to afford the crude product. The residue was purified by flash chromatography on silica gel eluting with dichloromethane/methanol (10/1) to afford (S)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (200.0 mg, 0.24 mmol, 48% yield) as a yellow solid. LC-MS: (ESI, m/z): 774.3 [M+H]⁺

Step 2: (S)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

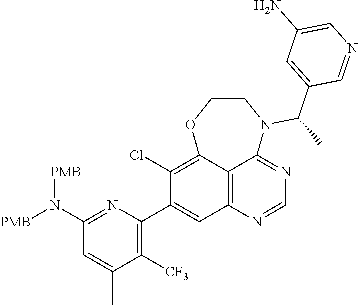

A solution of (S)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (200.0 mg, 0.24 mmol), N,N-diisopropylethylamine (66.7 mg, 0.51 mmol) and bis(2-oxo-3-oxazolidinyl)phosphinic chloride (85.4 mg, 0.32 mmol) in chloroform (3 mL) was stirred at 70° C. for 6 hours. After completion, the reaction mixture was diluted with dichloromethane. The resulting solution was washed with water, dried over anhydrous sodium sulfate and concentrated under vacuum. The residue was purified by flash chromatography on silica gel eluting with dichloromethane/methanol (10/1) to afford (S)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (90.0 mg, 0.11 mmol, 44.7% yield) as a yellow solid. LC-MS: (ESI, m/z): 756.3 [M+H]⁺

Step 3: (S)-6-(4-(1-(5-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

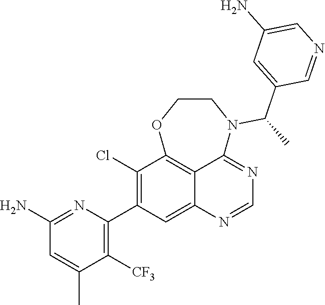 [SMILES: Cc1cc(N)nc(-c2cc3ncnc4c3c(c2Cl)OCCN4[C@@H](C)c2cncc(N)c2)c1C(F)(F)F]

A solution of (S)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (90.0 mg, 0.11 mmol) and trifluoroacetic acid (2 mL) was stirred at 25° C. for 24 hours. After completion, the reaction mixture was concentrated under reduced pressure to afford the crude product. The crude product was purified by Prep-HPLC with the following conditions: Column: YMC-Actus Triart C18 ExRS, 30*150 mm, 5 μm; Mobile Phase A: Water (10 mmol/L NH4HCO3), Mobile Phase B: ACN; Detector, UV 254 nm. RT: 8.32 to afford (S)-6-(4-(1-(5-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine (18.1 mg, 0.03 mmol, 29.5% yield). LC-MS: (ESI, m/z): 516.2 [M+H]⁺

Example 4: ¹H NMR (300 MHz, DMSO-d₆, ppm) δ 8.47 (d, J=1.7 Hz, 1H), 7.91-7.84 (m, 1H), 7.84-7.77 (m, 1H), 7.20 (d, J=1.1 Hz, 1H), 6.88 (d, J=8.4 Hz, 1H), 6.75 (s, 2H), 6.60-6.50 (m, 1H), 6.45 (s, 1H), 5.30 (s, 2H), 4.67-4.35 (m, 2H), 3.80-3.41 (m, 2H), 2.36 (d, J=2.3 Hz, 3H), 1.59 (dd, J=7.1, 2.4 Hz, 3H).

Example 5: (R)-6-(4-(1-(2-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

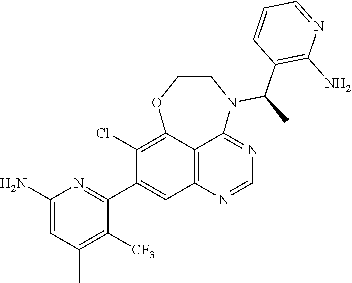 [SMILES: Cc1cc(N)nc(-c2cc3ncnc4c3c(c2Cl)OCCN4[C@H](C)c2cccnc2N)c1C(F)(F)F]

### Synthetic Route

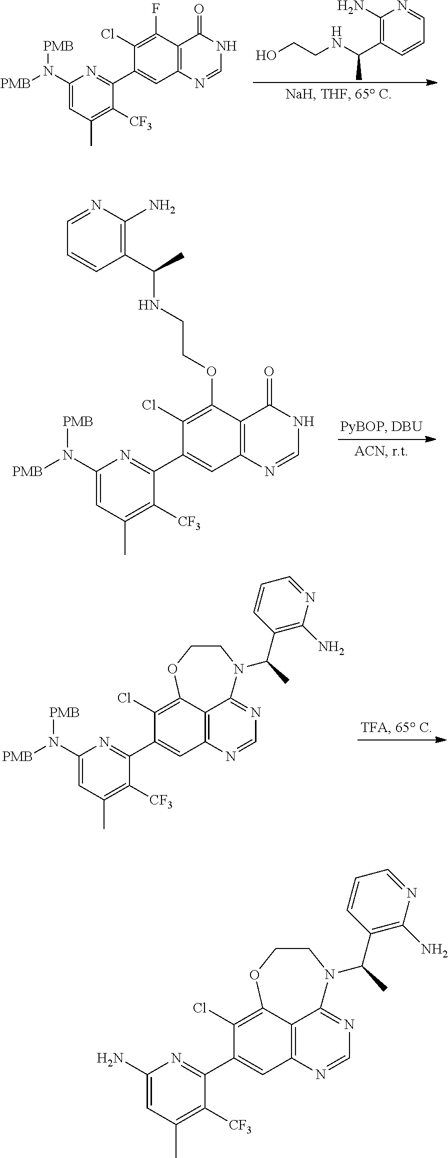

Step 1: (R)-5-(2-((1-(2-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one

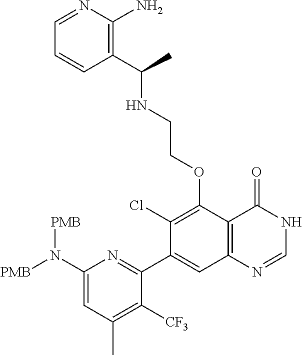

A solution of (R)-2-((1-(2-aminopyridin-3-yl)ethyl)amino)ethan-1-01 (133.0 mg, 0.73 mmol) and sodium hydride (46.9 mg, 1.92 mmol, 60% purity) in tetrachloroethylene (5 mL) was stirred at 0° C. for 20 minutes. Then 7-(6-(bis(4-methoxybenzyl)amino)-4-methyl-3-(trifluoromethyl)pyridin-2-yl)-6-chloro-5-fluoroquinazolin-4(3H)-one (300.0 mg, 0.48 mmol) was added and stirred at 65° C. for 1 hour. After completion, the reaction mixture was quenched with saturated ammonium chloride and concentrated under vacuum, diluted with water and extracted with ethyl acetate. The combined organic layers were dried over anhydrous sodium sulfate and concentrated under vacuum to afford the crude product. The residue was purified by flash chromatography on silica gel eluting with dichloromethane/methanol (10/1) to afford (R)-5-(2-((1-(2-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (200.0 mg, 0.23 mmol, 48% yield) as a yellow solid. LC-MS: (ESI, m/z): 774.3 [M+H]⁺

Step 2: (R)-6-(4-(1-(2-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

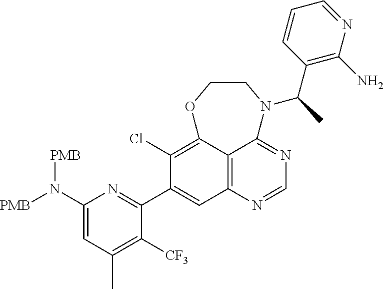

A solution of (R)-6-(4-(1-(2-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (250.0 mg, 0.32 mmol), 1,8-diazabicyclo[5.4.0]undec-7-ene (0.14 mL, 0.97 mmol) and benzotriazole-1-yl-oxytripyrrolidinophosphonium hexafluorophosphate (252.0 mg, 0.48 mmol) in Acetonitrile (3 mL) was stirred at for 1 hour. After completion, the reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford (R)-6-(4-(1-(2-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (90.0 mg, 0.12 mmol, 37.5% yield) as a yellow solid. LC-MS: (ESI, m/z): 756.3 [M+H]⁺

Step 3: (R)-6-(4-(1-(2-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

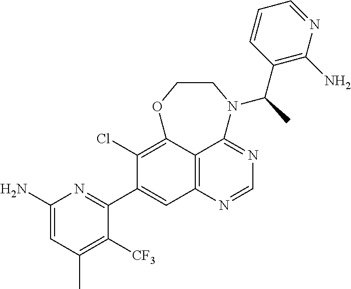 [SMILES: Cc1cc(N)nc(-c2cc3ncnc4c3c(c2Cl)OCCN4[C@H](C)c2cccnc2N)c1C(F)(F)F]

A solution of (R)-6-(4-(1-(2-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (90.0 mg, 0.1 mmol) and trifluoroacetic acid (2 mL) was stirred at 65° C. for 24 hours. After completion, the reaction mixture was concentrated under reduced pressure to afford the crude product. The crude product was purified by Prep-HPLC with the following conditions: Column: YMC-Actus Triart C18 ExRS, 30*150 mm, 5 μm; Mobile Phase A: Water (10 mmol/L NH4HCO3), Mobile Phase B: ACN; Detector, UV 254 nm. RT: 8.32 to afford (R)-6-(4-(1-(2-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine (18.1 mg, 0.03 mmol, 29.5% yield). LC-MS: (ESI, m/z): 516.2 [M+H]⁺

Example 5: ¹H NMR (400 MHz, DMSO-d₆, ppm) δ 8.51 (s, 1H), 7.99-7.92 (m, 1H), 7.67-7.60 (m, 1H), 7.19 (s, 1H), 6.75 (s, 2H), 6.70-6.62 (m, 1H), 6.45 (s, 2H), 5.80 (s, 1H), 5.73 (s, 1H), 4.59-4.45 (m, 1H), 4.36-4.24 (m, 1H), 3.93 (s, 1H), 3.74-3.60 (m, 1H), 2.35 (d, J=2.5 Hz, 3H), 1.59-1.51 (m, 3H).

Example 6: 6-(8-chloro-4-((5-(methylamino)pyridin-3-yl)methyl)-5,6-dihydro-4H-[1,4]oxazepino[5,6,7-de]quinazolin-9-yl)-4-methyl-5-(trifluoromethyl)pyridin-2-amine

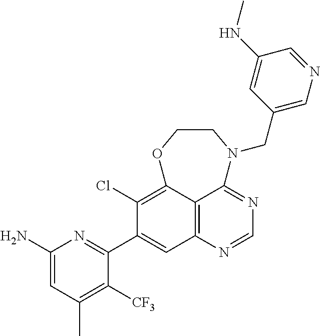 [SMILES: CNc1cncc(CN2CCOc3c(Cl)c(-c4nc(N)cc(C)c4C(F)(F)F)cc4ncnc2c34)c1]

### Synthetic Route

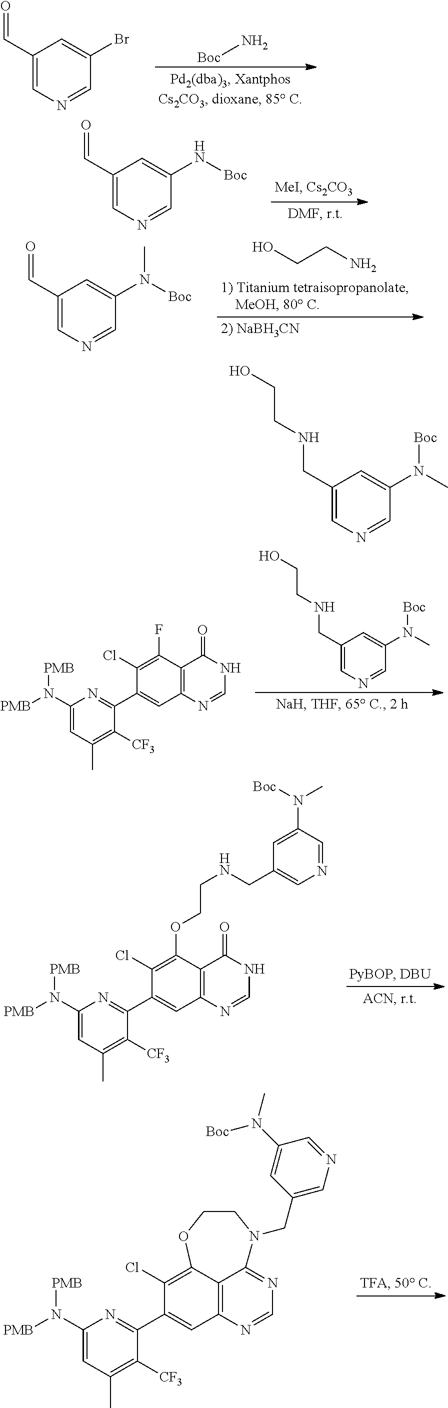

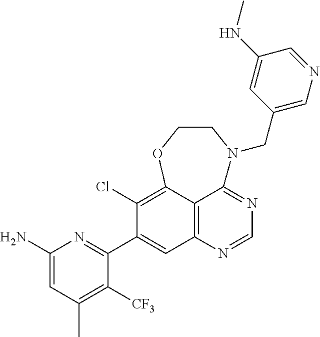 [SMILES: CNc1cncc(CN2CCOc3c(Cl)c(-c4nc(N)cc(C)c4C(F)(F)F)cc4ncnc2c34)c1]

Step 1: tert-butyl (5-formylpyridin-3-yl)carbamate

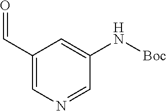 [SMILES: CC(C)(C)OC(=O)Nc1cncc(C=O)c1]

Under nitrogen, a solution of 5-bromonicotinaldehyde (2000.0 mg, 10.75 mmol), tert-butyl carbamate (1.89 g, 16.12 mmol), tris(dibenzylideneacetone)dipalladium-chloroform adduct (1.11 g, 1.08 mmol), 4,5-bis(diphenylphosphino)-9,9-dimethylxanthene (1.24 g, 2.150 mmol) and cesium carbonate (7.05 g, 21.50 mmol) in 1,4-dioxane (40 mL) was stirred at 85° C. for 3 hours. LC-MS showed the product formed and SM was consumed. After completion, the solution was concentrated under vacuum. The residue was purified by flash chromatography on silica gel eluting with petroleum ether/ethyl acetate (70/30) to afford tert-butyl N-(5-formyl-3-pyridyl)carbamate (1.88 g, 8.45 mmol, 78.7% yield) as a colorless solid. LC-MS: (ESI, m/z): 223.1 [M+H]⁺

Step 2: tert-butyl (5-formylpyridin-3-yl)(methyl)carbamate

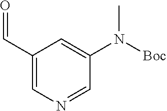 [SMILES: CN(C(=O)OC(C)(C)C)c1cncc(C=O)c1]

A solution of tert-butyl N-(5-formyl-3-pyridyl)carbamate (1.88 g, 8.46 mmol), iodomethane (1.32 g, 9.31 mmol) and cesium carbonate (5.54 g, 16.92 mmol) in N,N-dimethylformamide (20 mL) was stirred at 25° C. for 1 hour. After completion, the reaction mixture was diluted with ethyl acetate. The resulting solution was washed with water and dried over anhydrous sodium sulfate and concentrated under vacuum. The residue was purified by flash chromatography on silica gel eluting with petroleum ether/ethyl acetate (80/20) to afford tert-butyl (5-formylpyridin-3-yl)(methyl)carbamate (1.19 g, 5.03 mmol, 59.5% yield) as a yellow solid. LC-MS: (ESI, m/z): 237.1 [M+H]⁺

Step 3: tert-butyl (5-(((2-hydroxyethyl)amino)methyl)pyridin-3-yl)(methyl)carbamate

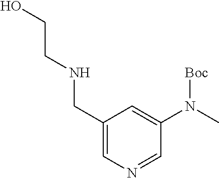 [SMILES: CN(C(=O)OC(C)(C)C)c1cncc(CNCCO)c1]

A solution of tert-butyl (5-formylpyridin-3-yl)(methyl)carbamate (1.19 g, 5.04 mmol), 2-aminoethanol (0.9 mL, 15.11 mmol) and sodiumcyanoborohydride (1.19 g, 18.94 mmol) in titanium(iv)isopropoxide (10.0 mL, 5.04 mmol) and methyl alcohol (10 mL) was stirred at 80° C. for 16 hours. After completion, the solvent was concentrated under vacuum. The residue was purified by flash chromatography on silica gel eluting with dichloromethane/methanol (95/5) to afford crude product. The residue was purified by flash chromatography on C18 gel eluting with methanol/water (25/75) to afford tert-butyl (5-(((2-hydroxyethyl)amino)methyl)pyridin-3-yl)(methyl)carbamate (1.10 g, 3.72 mmol, 73.9% yield) as a yellow oil. LC-MS: (ESI, m/z): 282.1 [M+H]⁺

Step 4: tert-butyl (5-(((2-((7-(6-(bis(4-methoxybenzyl)amino)-4-methyl-3-(trifluoromethyl)pyridin-2-yl)-6-chloro-4-oxo-3,4-dihydroquinazolin-5-yl)oxy)ethyl)amino)methyl)pyridin-3-yl)(methyl)carbamate

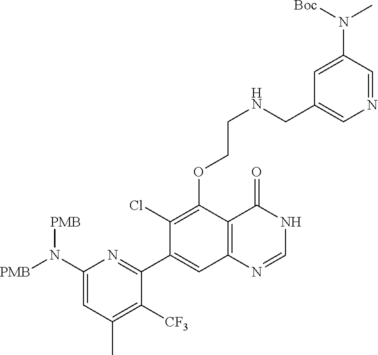

A solution of 7-(6-(bis(4-methoxybenzyl)amino)-4-methyl-3-(trifluoromethyl)pyridin-2-yl)-6-chloro-5-fluoroquinazolin-4(3H)-one (400.0 mg, 0.65 mmol), tert-butyl N-[5-[(2-hydroxyethylamino)methyl]-3-pyridyl]-N-methyl-carbamate (275.3 mg, 0.97 mmol) and sodium hydride (60%) (78.3 mg, 1.95 mmol) in tetrahydrofuran (3 mL) was stirred at 65° C. for 2 hours. After completion, the reaction mixture was quenched with saturated ammonium chloride, diluted with water and extracted with ethyl acetate. The combined organic layers were dried over anhydrous sodium sulfate and concentrated under vacuum. The residue was purified by flash chromatography on silica gel eluting with dichloromethane/methanol (10/1) to afford tert-butyl (5-(((2-((7-(6-(bis(4-methoxybenzyl)amino)-4-methyl-3-(trifluoromethyl)pyridin-2-yl)-6-chloro-4-oxo-3,4-dihydroquinazolin-5-yl)oxy)ethyl)amino)methyl)pyridin-3-yl)(methyl)carbamate (280.0 mg, 0.32 mmol, 49.1% yield) as a yellow solid. LC-MS: (ESI, m/z): 874.3 [M+H]⁺

Step 5: tert-butyl (5-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridin-3-yl)(methyl)carbamate

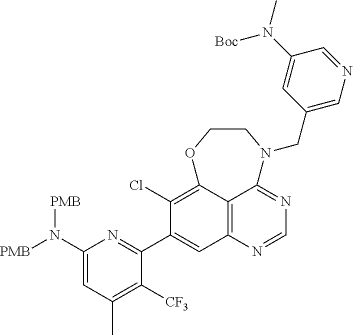

A solution of tert-butyl (5-(((2-((7-(6-(bis(4-methoxybenzyl)amino)-4-methyl-3-(trifluoromethyl)pyridin-2-yl)-6-chloro-4-oxo-3,4-dihydroquinazolin-5-yl)oxy)ethyl)amino)methyl)pyridin-3-yl)(methyl)carbamate (280.0 mg, 0.32 mmol), b1,8-diazabicyclo[5.4.0]undec-7-ene (0.1 mL, 1.28 mmol) and enzotriazole-1-yl-oxytripyrrolidinophosphonium hexafluorophosphate (1.0 mL, 1.44 mmol) in acetonitrile (3 mL) was stirred at 25° C. for 2 hours. After completion, the reaction mixture was concentrated under reduced pressure and diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford tert-butyl (5-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridin-3-yl)(methyl)carbamate (230.0 mg, 0.27 mmol, 85.5% yield) as a yellow solid. LC-MS: (ESI, m/z): 856.3 [M+H]⁺

Step 6: 6-(8-chloro-4-((5-(methylamino)pyridin-3-yl)methyl)-5,6-dihydro-4H-[1,4]oxazepino[5,6,7-de]quinazolin-9-yl)-4-methyl-5-(trifluoromethyl)pyridin-2-amine

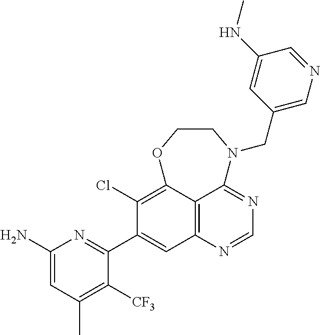 [SMILES: CNc1cncc(CN2CCOc3c(Cl)c(-c4nc(N)cc(C)c4C(F)(F)F)cc4ncnc2c34)c1]

A solution of tert-butyl (5-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridin-3-yl)(methyl)carbamate (230.0 mg, 0.27 mmol) and trifluoroacetic acid (3 mL) was stirred at 50° C. for 2 hours. After completion, the reaction mixture was concentrated under reduced pressure. The residue was purified by flash chromatography on silica gel eluting with dichloromethane/methanol (10/1) to afford the crude product. The crude product was purified by Prep-HPLC with the following conditions: Column: XBridge Prep OBD C18 Column, 30×150 mm 5 um; Mobile Phase A: Water (10 MMOL/L NH4HCO3), Mobile Phase B: ACN; Detector, UV 254 nm. RT: 6.5 to afford 6-(8-chloro-4-((5-(methylamino)pyridin-3-yl)methyl)-5,6-dihydro-4H-[1,4]oxazepino[5,6,7-de]quinazolin-9-yl)-4-methyl-5-(trifluoromethyl)pyridin-2-amine (27.8 mg, 0.05 mmol, 13% yield). LC-MS: (ESI, m/z): 516.1 [M+H]⁺

Example 6: ¹H NMR (300 MHz, DMSO-d₆, ppm) δ 8.44 (s, 1H), 7.84 (d, J=2.6 Hz, 1H), 7.79 (d, J=1.8 Hz, 1H), 7.19 (s, 1H), 6.89-6.81 (m, 1H), 6.78 (s, 2H), 6.46 (s, 1H), 5.91 (s, 1H), 5.14-4.98 (m, 2H), 4.71-4.55 (m, 2H), 3.97-3.88 (m, 2H), 2.67 (d, J=3.6 Hz, 3H), 2.36 (d, J=2.3 Hz, 3H).

Example 7: (R)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine

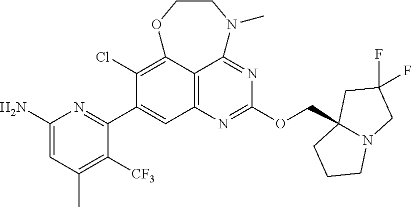 [SMILES: Cc1cc(N)nc(-c2cc3nc(OC[C@]45CCCN4CC(F)(F)C5)nc4c3c(c2Cl)OCCN4C)c1C(F)(F)F]

### Synthetic Route

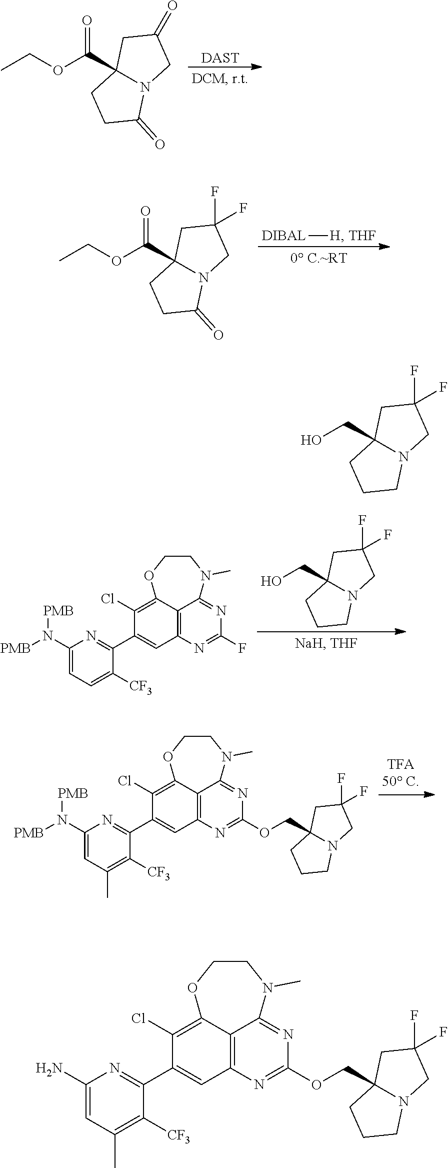

Step 1: ethyl (R)-2, 2-difluoro-5-oxotetrahydro-1H-pyrrolizine-7a(5H)-carboxylate

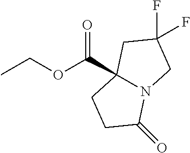 [SMILES: CCOC(=O)[C@]12CCC(=O)N1CC(F)(F)C2]

A solution of ethyl (R)-2, 5-dioxotetrahydro-1H-pyrrolizine-7a (5H)-carboxylate (20.00 g, 94.6 mmol) in dichloromethane (200 mL) was stirred at 0° C. for 5 minutes. Then diethylaminosulfur trifluoride (37.5 mL, 284.0 mmol) was added and stirred at room temperature for 6 hours. After reaction completion, the solvent was concentrated under vacuum. The residue was purified by flash chromatography on silica gel eluting with ethyl acetate/petroleum ether (1/10) to afford ethyl (R)-2, 2-difluoro-5-oxotetrahydro-1H-pyrrolizine-7a (5H)-carboxylate (15.70 g, 67.3 mmol, 71.1% yield) as a white solid. LC-MS: (ESI, m/z): 234.1 [M+H]⁺

Step 2: (R)-(2, 2-difluorotetrahydro-1H-pyrrolizin-7a (5H)-yl)methanol

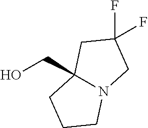 [SMILES: OC[C@]12CCCN1CC(F)(F)C2]

A solution of ethyl (R)-2,2-difluoro-5-oxotetrahydro-1H-pyrrolizine-7a(5H)-carboxylate (750.0 mg, 3.2 mmol) and lithium aluminum hydride (9.5 mL, 9.5 mol, 1 mol/L in THF) in tetrahydrofuran (8 mL) was stirred at 0° C. for 2 hours. After completion, the reaction mixture was quenched with sodium sulfate decahydrate and diluted with tetrahydrofuran. After filtration, the filtrate was concentrated under reduced pressure to afford (R)-(2, 2-difluorotetrahydro-1H-pyrrolizin-7a (5H)-yl)methanol (450 mg, crude). LC-MS: (ESI, m/z): 178.1 [M+H]⁺

Step 3: (R)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

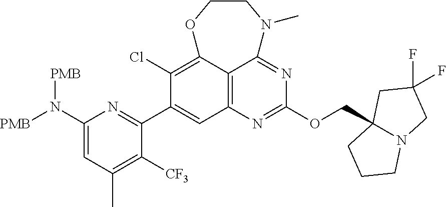

A solution of (R)-(2,2-difluorotetrahydro-1H-pyrrolizin-7a(5H)-yl)methanol (99.4 mg, crude) and sodium hydride (59.8 mg, 1.49 mmol, 60% purity) in tetrahydrofuran (3 mL) was stirred at 0° C. for 20 minutes. Then 6-(8-chloro-2-fluoro-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (250.0 mg, 0.37 mmol) was added and stirred at 25° C. for 2 hours. After completion, the reaction mixture was quenched with saturated ammonium chloride, diluted with water and extracted with ethyl acetate. The combined organic layers were dried over anhydrous sodium sulfate and concentrated under vacuum to afford (R)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (150.0 mg, 0.18 mmol, 48.6% yield) as a yellow solid. LC-MS: (ESI, m/z): 825.3 [M+H]⁺

Step 4: (R)-64(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine

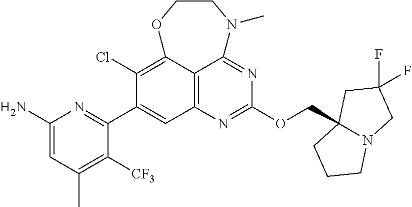 [SMILES: Cc1cc(N)nc(-c2cc3nc(OC[C@]45CCCN4CC(F)(F)C5)nc4c3c(c2Cl)OCCN4C)c1C(F)(F)F]

A solution of (R)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (150.0 mg, 0.18 mmol) in trifluoroacetic acid (2 mL) was stirred at 50° C. for 8 hours. After completion, the reaction mixture was concentrated under reduced pressure to afford the crude product. The crude product was purified by Prep-HPLC with the following conditions: Column: XBridge Prep OBD C18 Column, 30×150 mm 5 um; Mobile Phase A: Water (10 MMOL/L NH4HCO3), Mobile Phase B: ACN; Detector, UV 254 nm. RT: 6.5 to afford (R)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine (64.1 mg, 0.12 mmol, 60.3% yield). LC-MS: (ESI, m/z): 535.1 [M+H]⁺

Example 7: ¹H NMR (300 MHz, DMSO-d₆, ppm) δ 6.93 (s, 1H), 6.74 (d, J=2.1 Hz, 2H), 6.44 (s, 1H), 4.67-4.49 (m, 2H), 4.17-3.98 (m, 2H), 3.98-3.84 (m, 2H), 3.41 (s, 1H), 3.29 (s, 3H), 3.19-3.01 (m, 2H), 2.72 (d, J=8.6 Hz, 1H), 2.45-2.24 (m, 5H), 2.02 (d, J=5.1 Hz, 1H), 1.93-1.70 (m, 3H).

Example 8: 8-chloro-9-(6-fluoro-1-methyl-1H-indazol-7-yl)-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline

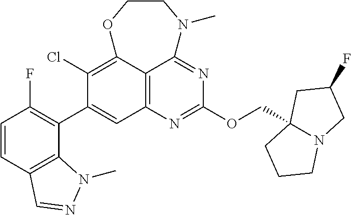 [SMILES: CN1CCOc2c(Cl)c(-c3c(F)ccc4cnn(C)c34)cc3nc(OC[C@@]45CCCN4C[C@H](F)C5)nc1c23]

### Synthesis Route

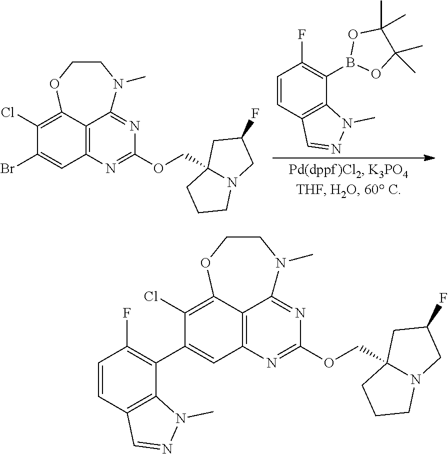

Step 1: 8-chloro-9-(6-fluoro-1-methyl-1H-indazol-7-yl)-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline

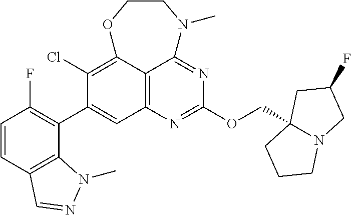 [SMILES: CN1CCOc2c(Cl)c(-c3c(F)ccc4cnn(C)c34)cc3nc(OC[C@@]45CCCN4C[C@H](F)C5)nc1c23]

Under nitrogen, a solution of 9-bromo-8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline (200.0 mg, 0.42 mmol), 6-fluoro-1-methyl-7-(4,4,5,5-tetramethyl-1,3,2-dioxaborolan-2-yl)indazole (117.0 mg, 0.42 mmol), potassium phosphate (179.7 mg, 0.84 mmol) and [1,1′-bis(diphenylphosphino)ferrocene]dichloropalladium(II) (31.4 mg, 0.042 mmol) in tetrahydrofuran (2.0 mL) and water (0.4 mL) was stirred at 60° C. for 1 hour. After completion, the solvent was concentrated under vacuum. The residue was purified by flash chromatography on silica gel eluting with dichloromethane/methanol (25/1) to afford 100 mg crude. The crude product was purified by Prep-HPLC with the following conditions: Column: XBridge Prep C18 OBD Column, 30*100 mm, 5 um; Mobile Phase A: Water (10 MMOL/L NH₄HCO₃), Mobile Phase B: ACN: Flow rate: 60 mL/min: Gradient: 50 B to 80 B in 7 min, 254/220 nm; RT1: 6.53 to afford 8-chloro-9-(6-fluoro-1-methyl-1H-indazol-7-yl)-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline (29.1 mg, 0.05 mmol, 12.7% yield). LC-MS: (ESI, m/z): 541.2 [M+H]⁺.

Example 8: ¹H NMR (300 MHz, DMSO-d6) δ 8.26 (s, 1H), 7.21 (d, J=8.2 Hz, 2H), 7.02 (dd, J=9.9, 7.9 Hz, 1H), 5.29 (d, J=54.3 Hz, 1H), 4.81-4.50 (m, 2H), 4.13-3.90 (m, 4H), 3.56 (s, 3H), 3.08 (d, J=28.7 Hz, 3H), 3.20-3.00 (m, 3H), 2.90-2.80 (m, 1H), 2.15 (d, J=5.2 Hz, 1H), 2.03 (d, J=11.7 Hz, 2H), 1.91-1.68 (m, 3H).

Example 9: 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridin-3-ol

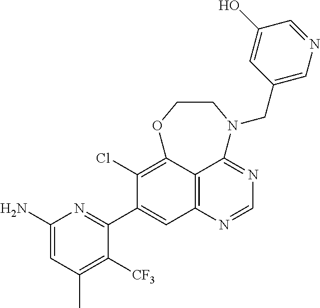 [SMILES: Cc1cc(N)nc(-c2cc3ncnc4c3c(c2Cl)OCCN4Cc2cncc(O)c2)c1C(F)(F)F]

### Synthesis Route

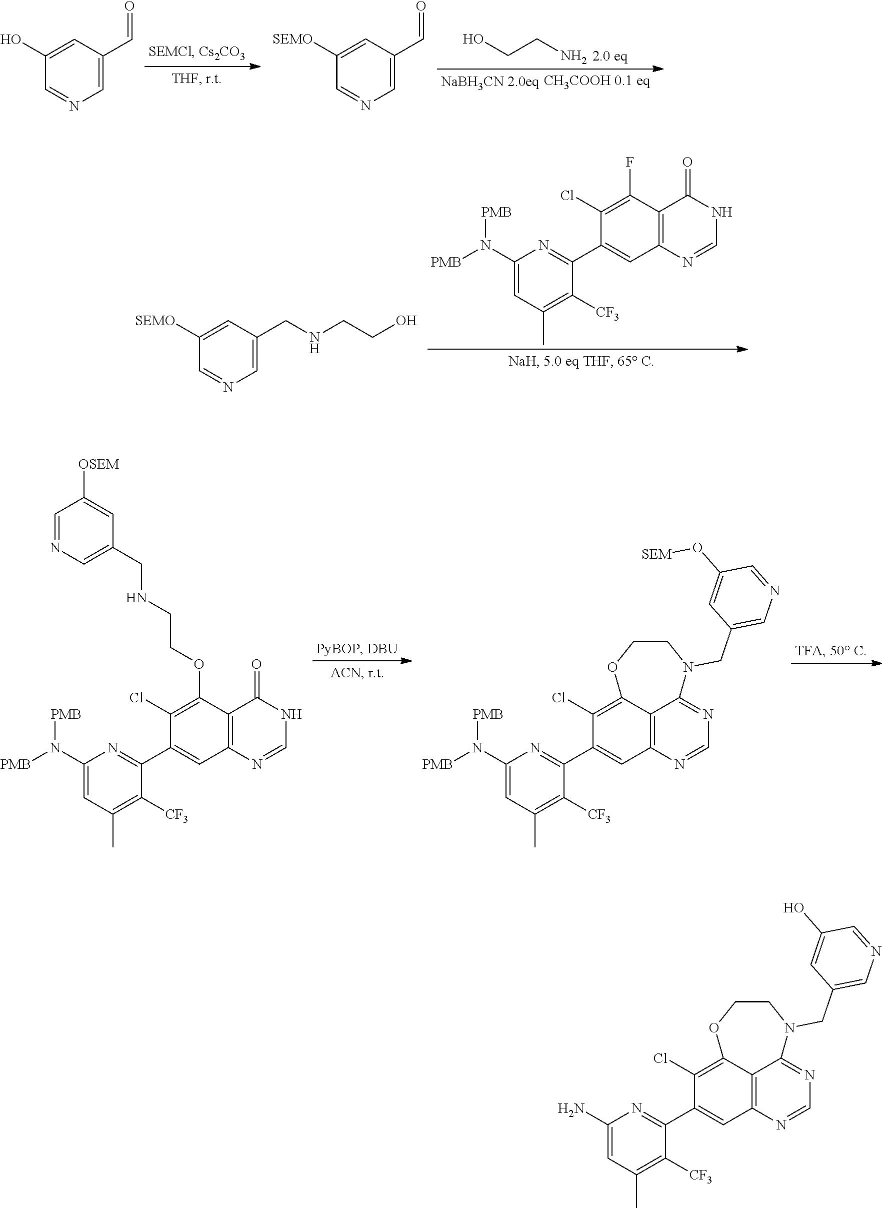

Step 1: 5-((2-(trimethylsilyl)ethoxy)methoxy)nicotinaldehyde

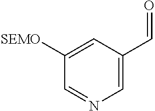

A solution of 5-hydroxynicotinaldehyde (2.00 g, 16.2 mmol) and cesium carbonate (10.6 g, 32.5 mmol) in tetrahydrofuran (20.0 mL) was stirred at 25° C. for 10 minutes. Then 2-(trimethylsilyl)ethoxymethyl chloride (2.8 mL, 16.2 mmol) was added and stirred at 25° C. for 3 hours. After reaction completion, the solvent was diluted by water and extracted with ethyl acetate. Then the organic layers were combined and dried over anhydrous sodium sulfate and concentrated under vacuum. The residue was purified by flash chromatography on silica gel eluting with methanol/dichloromethane (1/10) to afford 5-((2-(trimethylsilyl)ethoxy)methoxy)nicotinaldehyde (1.96 g, 7.2 mmol, 44.6% yield) as a yellow solid. LC-MS: (ESI, m/z): 254.1 [M+H]⁺

Step 2: 2-(((5-((2-(trimethylsilyl)ethoxy)methoxy)pyridin-3-yl)methyl)amino)ethan-1-ol

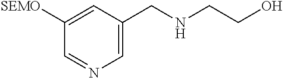

A solution of 2-aminoethanol (0.8 mL, 14.6 mmol) and acetic acid (0.1 mL, 0.7 mmol) in methyl alcohol (200.0 mL) was stirred at room temperature for 5 minutes. Then 5-((2-(trimethylsilyl)ethoxy)methoxy)nicotinaldehyde (1.86 g, 7.3 mmol) was added and stirred at room temperature for 2 hours. Then sodium cyanoborohydride (922.6 mg, 14.6 mmol) was added at 0° C. and stirred at room temperature for 2 hours. After reaction completion, the reaction was quenched by water. The solvent was concentrated under vacuum. The residue was purified by flash chromatography on silica gel eluting with methanol/dichloromethane (1/10) to afford 2-(((5-((2-(trimethylsilyl)ethoxy)methoxy)pyridin-3-yl)methyl)amino)ethan-1-ol (2.0 g, 6.4 mmol, 88.2% yield) as a colorless oil. LC-MS: (ESI, m/z): 299.2 [M+H]⁺

Step 3: 7-(6-(bis(4-methoxybenzyl)amino)-4-methyl-3-(trifluoromethyl)pyridin-2-yl)-6-chloro-5-(2-(((5-((2-(trimethylsilyl)ethoxy)methoxy)pyridin-3-yl)methyl)amino)ethoxy)quinazolin-4(3H)-one

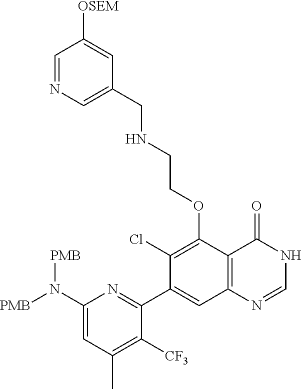

A solution of 2-(((5-((2-(trimethylsilyl)ethoxy)methoxy)pyridin-3-yl)methyl)amino)ethan-1-ol (400.0 mg, 1.30 mmol) and sodium hydride (64.3 mg, 2.60 mmol, 60% purity) in tetrahydrofuran (4.0 mL) was stirred at 0° C. for 5 minutes. Then 7-[6-[bis[(4-methoxyphenyl)methyl]amino]-4-methyl-3-(trifluoromethyl)-2-pyridyl]-6-chloro-5-fluoro-3H-quinazolin-4-one (410.7 mg, 0.65 mmol) was added and stirred at 65° C. for 1 hour. After reaction completion, the reaction was quenched by saturated ammonium chloride solution. The solvent was diluted by water and extracted with ethyl acetate. Then the organic layers were combined and dried over anhydrous sodium sulfate and concentrated under vacuum. The residue was purified by flash chromatography on silica gel eluting with methanol/dichloromethane (1/10) to afford 7-(6-(bis(4-methoxybenzyl)amino)-4-methyl-3-(trifluoromethyl)pyridin-2-yl)-6-chloro-5-(2-(((5-((2-(trimethylsilyl)ethoxy)methoxy)pyridin-3-yl)methyl)amino)ethoxy)quinazolin-4(3H)-one (464.0 mg, 0.52 mmol, 38.8% yield) as a white solid. LC-MS: (ESI, m/z): 891.5 [M+H]⁺

Step 4: 6-(8-chloro-4-((5-((2-(trimethylsilyl)ethoxy)methoxy)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

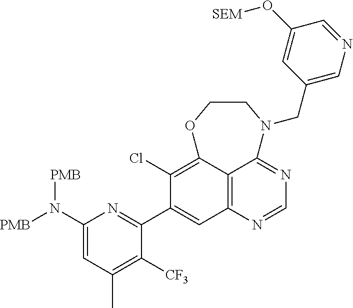

A solution of 7-(6-(bis(4-methoxybenzyl)amino)-4-methyl-3-(trifluoromethyl)pyridin-2-yl)-6-chloro-5-(2-(((5-((2-(trimethylsilyl)ethoxy)methoxy)pyridin-3-yl)methyl)amino)ethoxy)quinazolin-4(3H)-one (176.0 mg, 0.20 mmol), benzotriazole-1-yl-oxytripyrrolidinophosphonium hexafluorophosphate (154.1 mg, 0.30 mmol) and 1,8-diazabicycloundec-7-ene (0.1 mL, 0.57 mmol) in acetonitrile (2 mL) was stirred at room temperature for 1 hour. After completion, the solvent was concentrated under vacuum. The residue was purified by flash chromatography on silica gel eluting with methanol/dichloromethane (1/15) to afford 6-(8-chloro-4-((5-((2-(trimethylsilyl)ethoxy)methoxy)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (54.0 mg, 0.061 mmol, 28.9% yield) LC-MS: (ESI, m/z): 873.4 [M+H]⁺

Step 5: 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridin-3-ol

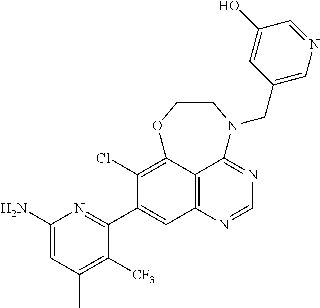 [SMILES: Cc1cc(N)nc(-c2cc3ncnc4c3c(c2Cl)OCCN4Cc2cncc(O)c2)c1C(F)(F)F]

A solution of 6-(8-chloro-4-((5-((2-(trimethylsilyl)ethoxy)methoxy)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.22 mmol) in 2,2,2-trifluoroacetic acid (3.0 mL) was stirred at 50° C. for 3 hours. After reaction completion, the solvent was concentrated under vacuum. The product was purified by Prep-HPLC with the following conditions (Column, XBridge Prep C18 OBD Column 19*15 mm 5 um C-0013: mobile phase, A: 1 mmol TFA in water, B: ACN and NH₄Cl % (51%-73% in 7 min); detector, UV 254 nm) to afford 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridin-3-ol (36.8 mg, 0.073 mmol, 32% yield). LC-MS: (ESI, m/z): 503.0 [M+H]⁺

Example 9: ¹H NMR (300 MHz, DMSO-d6, ppm) δ 10.03-9.70 (m, 1H), 8.60-8.35 (m, 1H), 8.15-7.84 (m, 2H), 7.19 (s, 1H), 7.16-7.09 (m, 1H), 6.76 (s, 2H), 6.44 (d, J=1.5 Hz, 1H), 5.08 (s, 2H), 4.79-4.48 (m, 2H), 3.94 (d, J=5.1 Hz, 2H), 2.40-2.30 (m, 3H).

Example 10: (S)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine

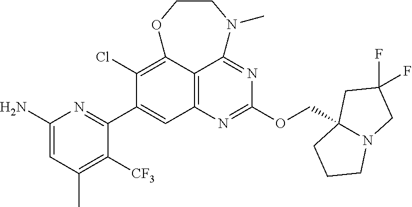 [SMILES: Cc1cc(N)nc(-c2cc3nc(OC[C@@]45CCCN4CC(F)(F)C5)nc4c3c(c2Cl)OCCN4C)c1C(F)(F)F]

### Synthesis Route:

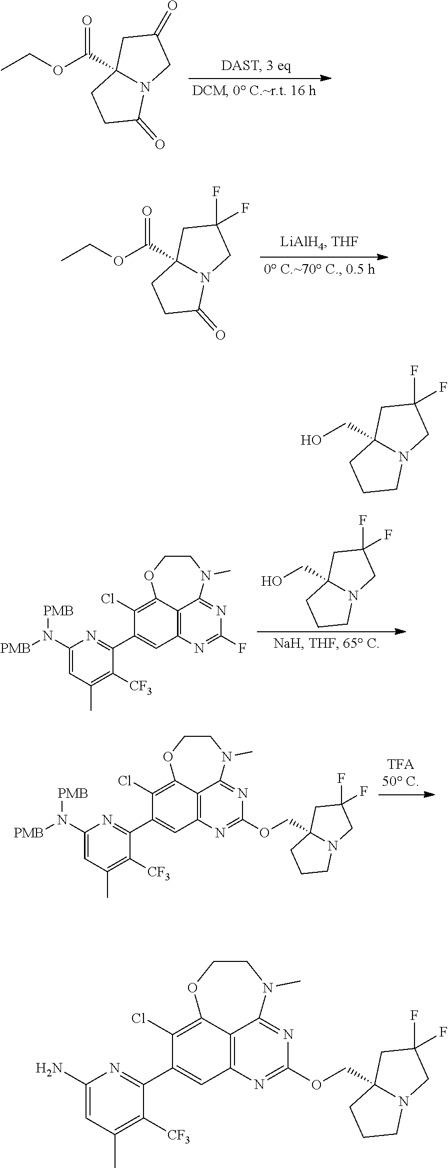

Step 1: ethyl (S)-2,2-difluoro-5-oxotetrahydro-1H-pyrrolizine-7a(5H)-carboxylate

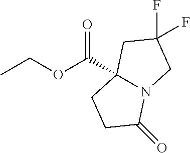 [SMILES: CCOC(=O)[C@@]12CCC(=O)N1CC(F)(F)C2]

Under nitrogen, a solution of ethyl (S)-2,5-dioxotetrahydro-1H-pyrrolizine-7a(5H)-carboxylate (15.00 g, 71.0 mmol) in dichloromethane (150.0 mL) was stirred at 0° C. for 10 minutes. Then diethylaminosulfur trifluoride (28.1 mL, 213.0 mmol) was added and stirred at 25° C. for 6 hours. After reaction completion, the reaction was quenched by ethanol, the solvent was concentrated under vacuum. The residue was purified by flash chromatography on silica gel eluting with ethyl acetate/petroleum ether (1/10) to afford ethyl (S)-2,2-difluoro-5-oxotetrahydro-1H-pyrrolizine-7a(5H)-carboxylate (7.29 g, 31.2 mmol, 44% yield) as a white solid. LC-MS: (ESI, m/z): 234.2 [M+H]⁺

Step 2: (S)-(2,2-difluorotetrahydro-1H-pyrrolizin-7a(5H)-yl)methanol

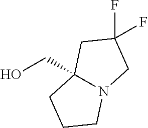 [SMILES: OC[C@@]12CCCN1CC(F)(F)C2]

A solution of ethyl (S)-2,2-difluoro-5-oxotetrahydro-1H-pyrrolizine-7a(5H)-carboxylate (7.2 g, 30.8 mmol) in tetrahydrofuran (100.0 mL) was stirred at 0° C. for 10 minutes. Then diisobutylaluminium hydride (13.10 g, 92.6 mmol, 1M in THF) was added and stirred at 70° C. for 30 minutes. After reaction completion, the reaction was quenched by sodium sulfate decahydrate (1.00 g). The resulting solution was filtrated, the filtrate was concentrated under reduced vacuum to afford (S)-(2,2-difluorotetrahydro-1H-pyrrolizin-7a(5H)-yl)methanol (4.70 g, crude) as a white oil. LC-MS: (ESI, m/z): 178.2 [M+H]⁺

Step 3: (S)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

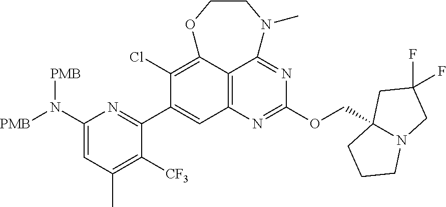

A solution of (S)-(2,2-difluorotetrahydro-1H-pyrrolizin-7a(5H)-yl)methanol (99.4 mg, 0.55 mmol) and sodium hydride (74.0 mg, 1.85 mmol, 60% purity) in tetrahydrofuran (4.0 mL) was stirred at room temperature for 5 minutes. Then 6-(8-chloro-3-fluoro-13-methyl-10-oxa-2,4,13-triazatricyclo[7.4.1.05,14]tetradeca-1,3,5(14),6,8-pentaen-7-yl)-N,N-bis[(4-methoxyphenyl)methyl]-4-methyl-5-(trifluoromethyl)pyridin-2-amine (250.0 mg, 0.35 mmol) was added and stirred at room temperature for 2 hours. After completion, the reaction was quenched by saturated ammonium chloride solution. The solvent was diluted by water and extracted with ethyl acetate. Then the organic layers were combined and dried over anhydrous sodium sulfate and concentrated under vacuum. The residue was purified by flash chromatography on silica gel eluting with methanol/dichloromethane (1/10) to afford (S)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (263.0 mg, 0.31 mmol, 88.5% yield) as a yellow solid. LC-MS: (ESI, m/z): 825.3 [M+H]⁺

Step 4: (S)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine

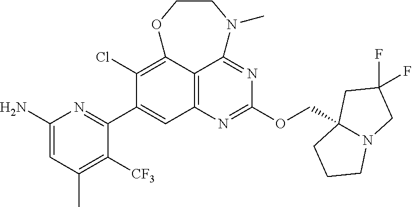 [SMILES: Cc1cc(N)nc(-c2cc3nc(OC[C@@]45CCCN4CC(F)(F)C5)nc4c3c(c2Cl)OCCN4C)c1C(F)(F)F]

A solution of (S)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (250.0 mg, 0.30 mmol) in 2,2,2-trifluoroacetic acid (4.0 mL) was stirred at 50° C. for 5 hours. After completion, the solvent was concentrated under vacuum. the resulting residue was purified by reverse phase chromatography (acetonitrile 0-40/0.1% NH₄Cl in water) to afford (S)-6-(8-chloro-2-((2,2-difluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine (93.0 mg, 0.15 mmol, 52.5% yield). LC-MS: (ESI, m/z): 585.0 [M+H]⁺

Example 10: ¹H NMR (300 MHz, DMSO-d₆) δ 6.80 (s, 1H), 6.62 (d, J=2.1 Hz, 2H), 6.39-6.21 (m, 1H), 4.57-4.32 (m, 2H), 4.08-3.68 (m, 4H), 3.28-3.17 (m, 4H), 3.08-2.84 (m, 2H), 2.66-2.49 (m, 1H), 2.36-2.09 (m, 5H), 1.96-1.83 (m, 1H), 1.71 (d, J=34.1 Hz, 3H).

Example 11: 6-(4-(1-(2-aminopyridin-3-yl)cyclopropyl)-8-chloro-5,6-dihydro-4H-[1,4]oxazepino[5,6,7-de]quinazolin-9-yl)-4-methyl-5-(trifluoromethyl)pyridin-2-amine

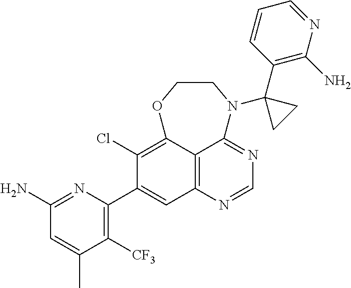 [SMILES: Cc1cc(N)nc(-c2cc3ncnc4c3c(c2Cl)OCCN4C2(c3cccnc3N)CC2)c1C(F)(F)F]

### Synthesis Route:

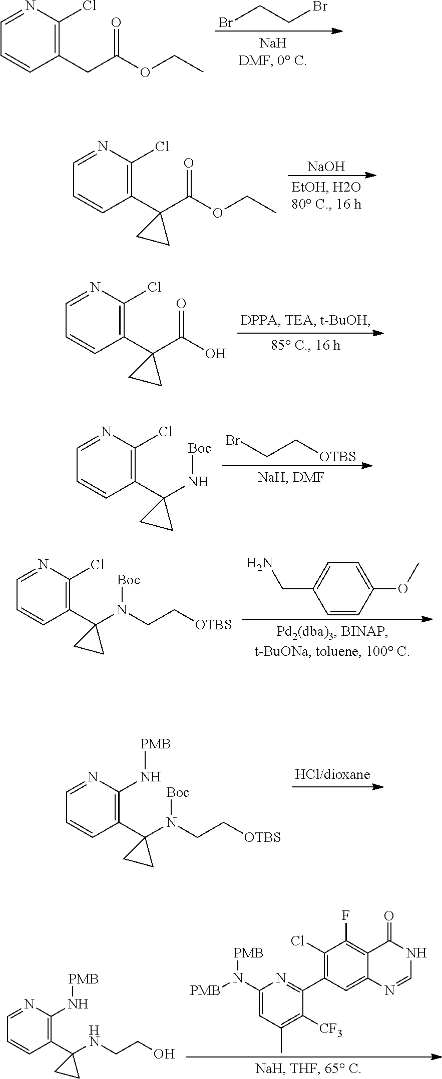

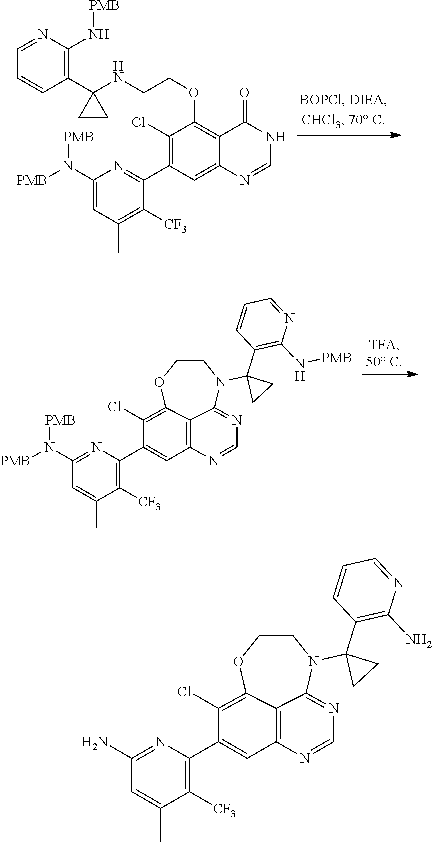

Step 1: ethyl 1-(2-chloropyridin-3-yl)cyclopropane-1-carboxylate

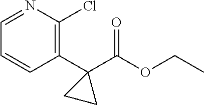

To a solution of ethyl 2-(2-chloro-3-pyridyl)acetate (1.00 g, 5.02 mmol) in N,N-dimethylformamide (25.0 mL) was added sodium hydride (800.0 mg, 20.08 mmol, 60% purity) and 1,2-dibromoethane (1.40 g, 7.48 mmol) at 0° C. And the solution was stirred for 2 h at 0° C. After completion, the reaction was quenched with saturated ammonium chloride. The resulting solution was diluted with water, extracted with ethyl acetate, washed with brine and concentrated. The residue was purified by flash chromatography on silica gel eluting with petroleum ether/ethyl acetate (4/1). This resulted in ethyl 1-(2-chloropyridin-3-yl)cyclopropane-1-carboxylate (660.0 mg, 2.92 mmol, 58.4% yield) as a colorless oil. LC-MS: (ESI, m/z): 226.1 [M+H]⁺

Step 2: 1-(2-chloropyridin-3-yl)cyclopropane-1-carboxylic acid

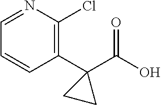 [SMILES: O=C(O)C1(c2cccnc2Cl)CC1]

A solution of ethyl 1-(2-chloropyridin-3-yl)cyclopropane-1-carboxylate (660.0 mg, 2.92 mmol) and sodium hydroxide (590.0 mg, 14.7 mmol) in ethanol (15.0 mL) and water (10.0 mL) was stirred at 80° C. for 24 h. After completion, the ethanol was removed under vacuum. The resulting solution was extracted with ethyl acetate, dried over anhydrous sodium sulfate and concentrated under vacuum to afford 1-(2-chloropyridin-3-yl)cyclopropane-1-carboxylic acid (570.0 mg, 2.87 mmol, 98.6% yield) as a white solid. LC-MS: (ESI, m/z): 198.0 [M+H]⁺

Step 3: tert-butyl (1-(2-chloropyridin-3-yl)cyclopropyl)carbamate

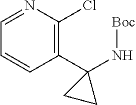 [SMILES: CC(C)(C)OC(=O)NC1(c2cccnc2Cl)CC1]

A mixture of 1-(2-chloropyridin-3-yl)cyclopropane-1-carboxylic acid (2.50 g, 12.62 mmol), triethylamine (4.00 g, 39.54 mmol) and diphenylphosphoryl azide (5.00 g, 18.16 mmol) in 2-methyl-2-propanol (50.0 mL) was stirred at 85° C. for 16 h. After completion, the resulting solution was diluted with water and extracted with ethyl acetate.

The organic layers were concentrated in vacuum. The residue was purified by flash chromatography on silica gel eluting with petroleum ether/ethyl acetate (7/3). This resulted in tert-butyl (1-(2-chloropyridin-3-yl)cyclopropyl)carbamate (3.00 g, 11.15 mmol, 88.2% yield) as a white solid. LC-MS: (ESI, m/z): 269.1 [M+H]⁺

Step 4: tert-butyl (2-((tert-butyldimethylsilyl)oxy)ethyl)(1-(2-chloropyridin-3-yl)cyclopropyl)carbamate

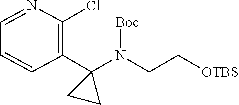 [SMILES: CC(C)(C)OC(=O)N(CCO[Si](C)(C)C(C)(C)C)C1(c2cccnc2Cl)CC1]

To a mixture of tert-butyl N-[1-(2-chloro-3-pyridyl)cyclopropyl]carbamate (3.20 g, 11.90 mmol) in N,N-dimethylformamide (50.0 mL) was added sodium hydride (1.50 g, 37.58 mmol, 60% purity) at room temperature and stirred for 1h, then (2-bromoethoxy)-tert-butyldimethylsilane (3.8 mL, 17.85 mmol) was added and stirred for 4 h. After completion, the reaction was quenched with saturated ammonium chloride. The resulting solution was extracted with ethyl acetate. The organic layers was washed with brine, dried over sodium sulfate and concentrated. The residue was purified by flash chromatography on silica gel eluting with petroleum ether/ethyl acetate (80/20). This resulted in tert-butyl (2-((tert-butyldimethylsilyl)oxy)ethyl)(1-(2-chloropyridin-3-yl)cyclopropyl)carbamate (4.00 g, 9.36 mmol, 78.7% yield) as a yellow oil. LC-MS: (ESI, m/z): 427.1 [M+H]⁺

Step 5: tert-butyl (2-((tert-butyldimethylsilyl)oxy)ethyl)(1-(2-((4-methoxybenzyl)amino)pyridin-3-yl)cyclopropyl)carbamate

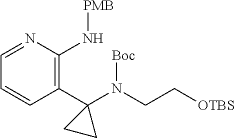

A mixture of 4-methoxybenzylamine (0.3 mL, 2.30 mmol), tert-butyl N-[2-[tert-butyl(dimethyl)silyl]oxyethyl]-n-[1-(2-chloro-3-pyridyl)cyclopropyl]carbamate (500.0 mg, 1.15 mmol), tris(dibenzylideneacetone)dipalladium (110.0 mg, 0.1 mmol), 1.1′-binaphthyl-2.2′-diphenyl phosphine (150.0 mg, 0.23 mmol) and sodium tert-butoxide (340.0 mg, 3.51 mmol) in toluene (8.0 mL) was stirred at 100° C. for 2 h. After completion, the resulting solution was diluted with water. The resulting solution was extracted with ethyl acetate. The organic layers was washed with brine, dried over sodium sulfate and concentrated. The residue was purified by flash chromatography on silica gel eluting with petroleum ether/ethyl acetate (4/1). This resulted in tert-butyl (2-((tert-butyldimethylsilyl)oxy)ethyl)(1-(2-((4-methoxybenzyl)amino)pyridin-3-yl)cyclopropyl)carbamate (560.0 mg, 1.06 mmol, 90.6% yield) as a yellow oil. LC-MS: (ESI, m/z): 528.3 [M+H]⁺

Step 6: 2-((1-(2-((4-methoxybenzyl)amino)pyridin-3-yl)cyclopropyl)amino)ethan-1-ol

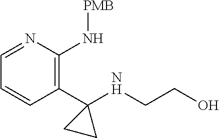

A solution of tert-butyl (2-((tert-butyldimethylsilyl)oxy)ethyl)(1-(2-((4-methoxybenzyl)amino)pyridin-3-yl)cyclopropyl)carbamate (500.0 mg, 1.06 mmol) and hydrochloric acid (1M in 1,4-dioxane) (6.0 mL) was stirred at room temperature for 4 hours. After completion, LC-MS showed the product formed and SM was consumed. The crude product (600 mg, crude) would be directly used in the next step without purification. LC-MS: (ESI, m/z): 314.2 [M+H]⁺

Step 7: 7-(6-(bis(4-methoxybenzyl)amino)-4-methyl-3-(trifluoromethyl)pyridin-2-yl)-6-chloro-5-(2-((1-(2-((4-methoxybenzyl)amino)pyridin-3-yl)cyclopropyl)amino)ethoxy)quinazolin-4(3H)-one

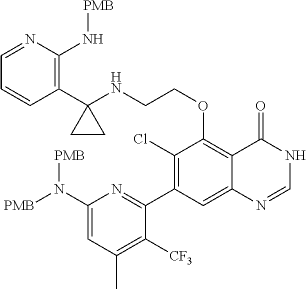

A solution of 7-[6-[bis[(4-methoxyphenyl)methyl]amino]-4-methyl-3-(trifluoromethyl)-2-pyridyl]-6-chloro-5-fluoro-3H-quinazolin-4-one (300.0 mg, 0.49 mmol), 2-((1-(2-((4-methoxybenzyl)amino)pyridin-3-yl)cyclopropyl)amino)ethan-1-ol (153.37 mg, 0.49 mmol) and Sodium hydride (58.7 mg, 2.45 mmol, 60% purity) in tetrahydrofuran (4.0 mL) was stirred at 65° C. for 5 hours. After completion, the solvent was concentrated under vacuum. The residue was purified by flash chromatography on silica gel eluting with methanol/dichloromethane (1/10) to afford 7-(6-(bis(4-methoxybenzyl)amino)-4-methyl-3-(trifluoromethyl)pyridin-2-yl)-6-chloro-5-(2-((1-(2-((4-methoxybenzyl)amino)pyridin-3-yl)cyclopropyl)amino)ethoxy)quinazolin-4(3H)-one (300.0 mg, 0.33 mmol, 51.4% yield) as a yellow solid. LC-MS: (ESI, m/z): 906.4 [M+H]⁺

Step 8: 6-(8-chloro-4-(1-(2-((4-methoxybenzyl)amino)pyridin-3-yl)cyclopropyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

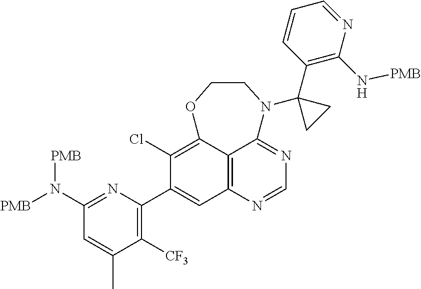

A solution of 7-(6-(bis(4-methoxybenzyl)amino)-4-methyl-3-(trifluoromethyl)pyridin-2-yl)-6-chloro-5-(2-((1-(2-((4-methoxybenzyl)amino)pyridin-3-yl)cyclopropyl)amino)ethoxy)quinazolin-4(3H)-one (400.0 mg, 0.44 mmol), bis(2-oxo-3-oxazolidinyl)phosphinic chloride (168.5 mg, 0.66 mmol) and N,N-diisopropylethylamine (171.1 mg, 1.32 mmol) in chloroform (2.0 mL) was stirred at 70° C. for 3 hours. After completion, the solvent was concentrated under vacuum. The residue was purified by flash chromatography on silica gel eluting with ethyl acetate/petroleum ether (2/1) to afford 6-(8-chloro-4-(1-(2-((4-methoxybenzyl)amino)pyridin-3-yl)cyclopropyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (230.0 mg, 0.26 mmol, 58.7% yield) as a yellow solid. LC-MS: (ESI, m/z): 888.4 [M+H]⁺

Step 9: 6-(4-(1-(2-aminopyridin-3-yl)cyclopropyl)-8-chloro-5,6-dihydro-4H-[1,4]oxazepino[5,6,7-de]quinazolin-9-yl)-4-methyl-5-(trifluoromethyl)pyridin-2-amine

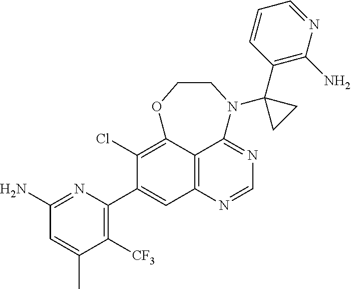 [SMILES: Cc1cc(N)nc(-c2cc3ncnc4c3c(c2Cl)OCCN4C2(c3cccnc3N)CC2)c1C(F)(F)F]

A solution of 6-(8-chloro-4-(1-(2-((4-methoxybenzyl)amino)pyridin-3-yl)cyclopropyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (220.0 mg, 0.2 mmol) in 2,2,2-trifluoroacetic acid (4.0 mL) was stirred at 70° C. for 4 hours. After completion, the solvent was concentrated under vacuum. The residue was purified by flash chromatography on silica gel eluting with methanol/dichloromethane (1/10) to afford 6-(4-(1-(2-aminopyridin-3-yl)cyclopropyl)-8-chloro-5,6-dihydro-4H-[1,4]oxazepino[5,6,7-de]quinazolin-9-yl)-4-methyl-5-(trifluoromethyl)pyridin-2-amine (46.3 mg, 0.087 mmol, 35.3% yield). LC-MS: (ESI, m/z): 528.1 [M+H]⁺.

Example 11: ¹H NMR (300 MHz, DMSO-d6) δ8.52 (s, 1H), 7.91 (dd, J=4.9, 1.8 Hz, 1H), 7.78 (dd, J=7.4, 1.9 Hz, 1H), 7.19 (s, 3H), 6.77 (s, 2H), 6.55 (dd, J=7.4, 4.8 Hz, 1H), 6.45 (s, 1H), 4.61 (dd, J=32.8, 11.4 Hz, 2H), 4.05 (d, J=21.5 Hz, 2H), 2.35 (q, J=2.1 Hz, 3H), 1.56 (s, 2H), 1.40-1.20 (m, 2H).

Example 12: 6-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridin-2(1H)-one

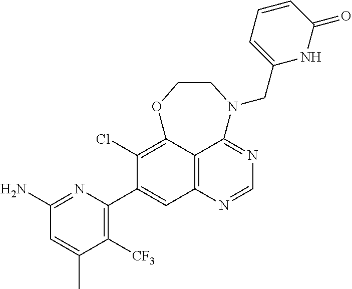 [SMILES: Cc1cc(N)nc(-c2cc3ncnc4c3c(c2Cl)OCCN4Cc2cccc(=O)[nH]2)c1C(F)(F)F]

### Synthesis Route:

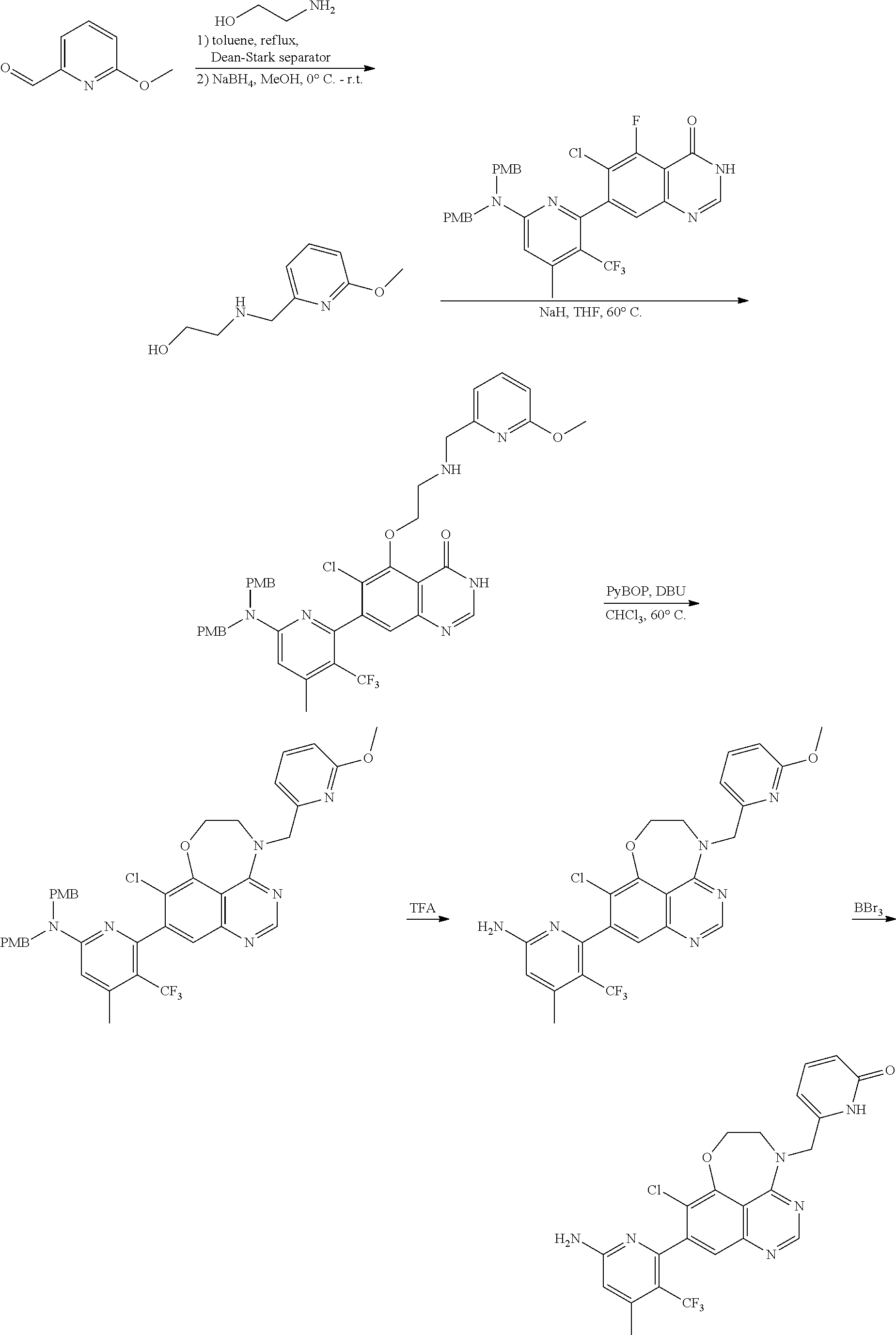

Step 1: 2-(((6-methoxypyridin-2-yl)methyl)amino)ethan-1-ol

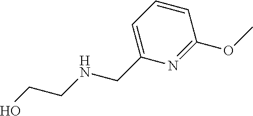

In dean-Stark separator, a solution of ethanolamine (1.3 mL, 21.78 mmol) and 6-methoxy-2-pyridinecarbaldehyde (1.7 mL, 14.54 mmol) in toluene (20.0 mL) was stirred at 120° C. for 6 hours. Then reaction solvent was concentrated under vacuum. Then sodium borohydride (1.9 g, 52.54 mmol) and methyl alcohol (20.0 mL) was added to reaction mixture at 0° C. stirred for 1 hour. After completion, the solvent was concentrated under vacuum. The residue was purified by flash chromatography on silica gel eluting with methanol/dichloromethane (1/10) to afford 2-(((6-methoxypyridin-2-yl)methyl)amino)ethan-1-ol (1.12 g, 6.12 mmol, 41.6% yield) as a white solid. LCMS (ESI, m/z): 183.2 [M+H]⁺.

Step 2: 7-(6-(bis(4-methoxybenzyl)amino)-4-methyl-3-(trifluoromethyl)pyridin-2-yl)-6-chloro-5-(2-(((6-methoxypyridin-2-yl)methyl)amino)ethoxy)quinazolin-4(3H)-one

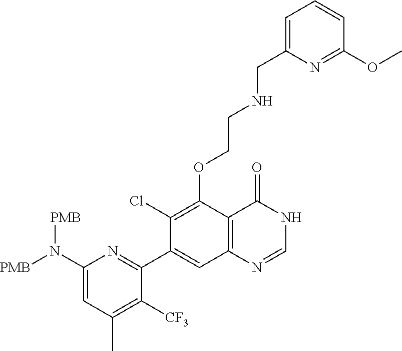

A solution of 2-(((6-methoxypyridin-2-yl)methyl)amino)ethan-1-ol (178.3 mg, 0.95 mmol) and sodium hydride (78.3 mg, 3.2 mmol, 60% purity) in tetrahydrofuran (5.0 mL) was stirred at 60° C. for 5 minutes. Then 7-[6-[bis[(4-methoxyphenyl)methyl]amino]-4-methyl-3-(trifluoromethyl)-2-pyridyl]-6-chloro-5-fluoro-3H-quinazolin-4-one (400.0 mg, 0.65 mmol) was added and stirred at 60° C. for 3 hours. After completion, the reaction was quenched by dilute hydrochloric acid. The solvent was diluted by water and extracted with ethyl acetate. Then the organic layers were combined and dried over anhydrous sodium sulfate and concentrated under vacuum. The residue was purified by flash chromatography on silica gel eluting with methanol/dichloromethane (1/30) to afford 7-(6-(bis(4-methoxybenzyl)amino)-4-methyl-3-(trifluoromethyl)pyridin-2-yl)-6-chloro-5-(2-(((6-methoxypyridin-2-yl)methyl)amino)ethoxy)quinazolin-4(3H)-one (430.0 mg, 0.55 mmol, 81.6% yield) as a white solid. LCMS (ESI, m/z): 775.2 [M+H]⁺.

Step 3: 6-(8-chloro-4-((6-methoxypyridin-2-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

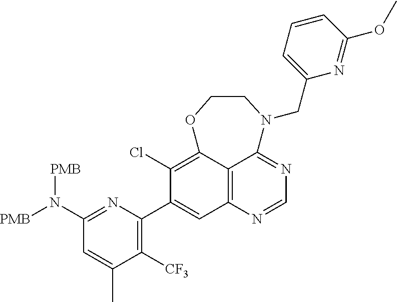

A solution of 7-(6-(bis(4-methoxybenzyl)amino)-4-methyl-3-(trifluoromethyl)pyridin-2-yl)-6-chloro-5-(2-(((6-methoxypyridin-2-yl)methyl)amino)ethoxy)quinazolin-4(3H)-one (420.0 mg, 0.52 mmol), benzotriazole-1-yl-oxytripyrrolidinophosphonium hexafluorophosphate (422.9 mg, 0.81 mmol) and 1,8-diazabicyclo[5.4.0]undec-7-ene (0.2 mL, 1.64 mmol) in chloroform (5.0 mL) was stirred at 60° C. for 2 hours. After completion, the solvent was concentrated under vacuum. The residue was purified by flash chromatography on silica gel eluting with ethyl acetate/petroleum ether (1/3) to afford 6-(8-chloro-4-((6-methoxypyridin-2-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (317.0 mg, 0.41 mmol, 76.5% yield) as a white solid. LCMS (ESI, m/z): 757.2 [M+H]⁺.

Step 4: 6-(8-chloro-4-((6-methoxypyridin-2-yl)methyl)-5,6-dihydro-4H-[1,4]oxazepino[5,6,7-de]quinazolin-9-yl)-4-methyl-5-(trifluoromethyl)pyridin-2-amine

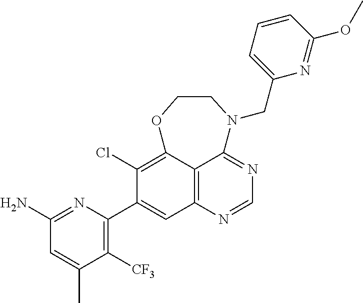 [SMILES: COc1cccc(CN2CCOc3c(Cl)c(-c4nc(N)cc(C)c4C(F)(F)F)cc4ncnc2c34)n1]

A solution of 6-(8-chloro-4-((6-methoxypyridin-2-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (300.0 mg, 0.39 mmol) in 2,2,2-trifluoroacetic acid (4.0 mL) was stirred at 50° C. for 3 hours. After completion, the solvent was concentrated under vacuum. The resulting residue was purified by reverse phase chromatography (acetonitrile 0-40/0.1% NH₄HCO₃ in water) to afford 6-(8-chloro-4-((6-methoxypyridin-2-yl)methyl)-5,6-dihydro-4H-[1,4]oxazepino[5,6,7-de]quinazolin-9-yl)-4-methyl-5-(trifluoromethyl)pyridin-2-amine (172.0 mg, 0.33 mmol, 84% yield) as a white solid. LCMS (ESI, m/z): 517.1 [M+H]⁺

Step 5: 6-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridin-2(1H)-one

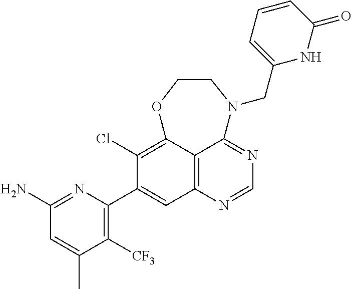 [SMILES: Cc1cc(N)nc(-c2cc3ncnc4c3c(c2Cl)OCCN4Cc2cccc(=O)[nH]2)c1C(F)(F)F]

A solution of 6-(8-chloro-4-((6-methoxypyridin-2-yl)methyl)-5,6-dihydro-4H-[1,4]oxazepino[5,6,7-de]quinazolin-9-yl)-4-methyl-5-(trifluoromethyl)pyridin-2-amine (150.0 mg, 0.29 mmol) and boron tribromide (726.9 mg, 2.92 mmol) in 1,2-dichloroethane (3.0 mL) was stirred at 80° C. for 10 hours. After completion, the reaction was quenched by water. The solvent was concentrated under vacuum. The resulting residue was purified by reverse phase chromatography (acetonitrile 0-40/0.1% NH₄Cl in water) to afford to afford 6-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridin-2(1H)-one (68.2 mg, 0.13 mmol, 46.3% yield). LCMS (ESI, m/z): 503.1 [M+H]⁺.

Example 12: ¹H NMR (300 MHz, DMSO-d5) δ 11.62 (s, 1H), 8.40 (s, 1H), 7.40-7.23 (m, 1H), 7.19 (s, 1H), 6.75 (s, 2H), 6.44 (s, 1H), 6.20 (d, J=9.1 Hz, 1H), 6.06 (s, 1H), 4.96-4.78 (m, 2H), 4.75-4.60 (m, 2H), 4.07-3.90 (m, 2H), 2.34 (d, J=2.4 Hz, 3H).

Example 13: 3-(9-(6-amino-4-methyl-3-(trifluoromethyl)pyridin-2-yl)-8-chloro-5,6-dihydro-4H-[1,4]oxazepino[5,6,7-de]quinazolin-4-yl)propanenitrile

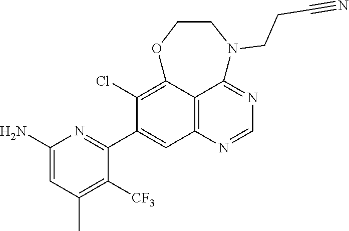 [SMILES: Cc1cc(N)nc(-c2cc3ncnc4c3c(c2Cl)OCCN4CCC#N)c1C(F)(F)F]

### Synthetic Route

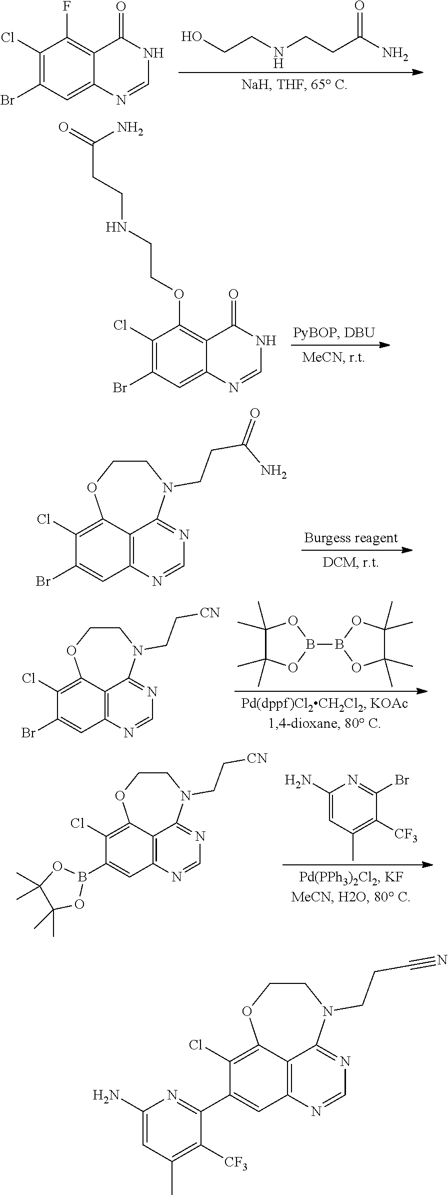

Step 1: 3-((2-((7-bromo-6-chloro-4-oxo-3,4-dihydroquinazolin-5-yl)oxy)ethyl)amino)propanamide

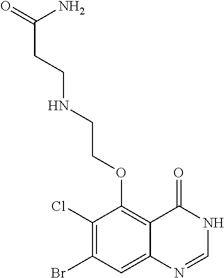 [SMILES: NC(=O)CCNCCOc1c(Cl)c(Br)cc2nc[nH]c(=O)c12]

A solution of 7-bromo-6-chloro-5-fluoro-3H-quinazolin-4-one (1.50 g, 5.41 mmol) in tetrahydrofuran (40 mL) was stirred at 65° C. for 5 minutes. Then sodium hydride (0.65 g, 16.22 mmol, 60% purity) and 3-(2-hydroxyethylamino)propanamide (1.43 g, 10.82 mmol) was added and stirred at 65° C. for 3 hours. After completion, the reaction mixture was adjusted to pH 7-8 with hydrochloric acid (1N). The solvent was concentrated under vacuum. The residue was purified by flash chromatography on silica gel eluting with acetonitrile/water (1/4) to afford 3-((2-((7-bromo-6-chloro-4-oxo-3,4-dihydroquinazolin-5-yl)oxy)ethyl)amino)propanamide (1.50 g, 3.85 mmol, 71.2% yield) as a white solid. LC-MS: (ESI, m/z): 389.6 [M+H]⁺.

Step 2: 3-(9-bromo-8-chloro-5,6-dihydro-4H-[1,4]oxazepino[5,6,7-de]quinazolin-4-yl)propanamide

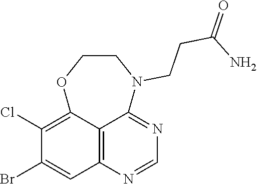 [SMILES: NC(=O)CCN1CCOc2c(Cl)c(Br)cc3ncnc1c23]

A solution of 3-((2-((7-bromo-6-chloro-4-oxo-3,4-dihydroquinazolin-5-yl)oxy)ethyl)amino)propanamide (1.50 g, 3.85 mmol) and 1,8-diazabicyclo[5.4.0]undec-7-ene (1.7 g, 11.11 mmol) in acetonitrile (20 mL) was stirred at 25° C. for 5 minutes. Then benzotriazole-1-yl-oxytripyrrolidinophosphonium hexafluorophosphate (2.40 g, 4.66 mmol) was added and stirred at 25° C. for 2 hours. After completion, the reaction mixture was diluted with ethyl acetate, washed with water and the organic layers were combined. The organic layer was dried over anhydrous sodium sulfate and concentrated under vacuum. The residue was purified by flash chromatography on silica gel eluting with dichloromethane/methanol (10/1) to afford 3-(9-bromo-8-chloro-5,6-dihydro-4H-[1,4]oxazepino[5,6,7-de]quinazolin-4-yl)propanamide (980.0 mg, 2.63 mmol, 68.5% yield) as a yellow solid. LC-MS: (ESI, m/z): 371.6 [M+H]⁺.

Step 3: 3-(9-bromo-8-chloro-5,6-dihydro-4H-[1,4]oxazepino[5,6,7-de]quinazolin-4-yl)propanenitrile

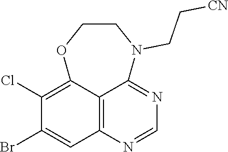 [SMILES: N#CCCN1CCOc2c(Cl)c(Br)cc3ncnc1c23]

Under nitrogen, a solution of 3-(9-bromo-8-chloro-5,6-dihydro-4H-[1,4]oxazepino[5,6,7-de]quinazolin-4-yl)propanamide (980.0 mg, 2.63 mmol) in dichloromethane (15 mL) was added Burgess reagent (1.25 g, 5.27 mmol) at 25° C. The resulting solution was stirred for time at 25° C. After completion, the solvent was concentrated under vacuum. The residue was purified by flash chromatography on silica gel eluting with dichloromethane/methanol (10/1) to afford 3-(9-bromo-8-chloro-5,6-dihydro-4H-[1,4]oxazepino[5,6,7-de]quinazolin-4-yl)propanenitrile (900.0 mg, 2.54 mmol, 96.5% yield) as a yellow solid. LC-MS: (ESI, m/z): 353.6 [M+H]⁺.

Step 4: 3-(8-chloro-9-(4,4,5,5-tetramethyl-1,3,2-dioxaborolan-2-yl)-5,6-dihydro-4H-[1,4]oxazepino[5,6,7-de]quinazolin-4-yl)propanenitrile

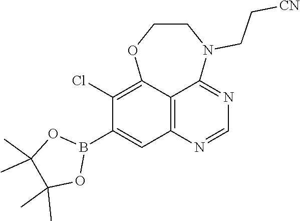 [SMILES: CC1(C)OB(c2cc3ncnc4c3c(c2Cl)OCCN4CCC#N)OC1(C)C]

Under nitrogen, a solution of 3-(9-bromo-8-chloro-5,6-dihydro-4H-[1,4]oxazepino[5,6,7-de]quinazolin-4-yl)propanenitrile (600.0 mg, 1.70 mmol), potassium acetate (333.0 mg, 3.3 mmol), [1,1′-bis(diphenylphosphino)ferrocene]dichloropalladium(II) (124.1 mg, 0.17 mmol) and bis(pinacolato)diboron (1292.6 mg, 5.09 mmol) in 1,4-dioxane (4 mL) was added at 80° C. for 12 hours. After completion, the solvent was concentrated under vacuum. The reaction mixture was diluted with dichloromethane. After filtration, the filtrate was concentrated under reduced pressure. The reaction mixture was diluted with petroleum ether. After filtration, the crude product (800 mg, crude) would be directly used in the next step without purification. LC-MS: (ESI, m/z): 400.7 [M+H]⁺.

Step 5: 3-(9-(6-amino-4-methyl-3-(trifluoromethyl)pyridin-2-yl)-8-chloro-5,6-dihydro-4H-[1,4]oxazepino[5,6,7-de]quinazolin-4-yl)propanenitrile

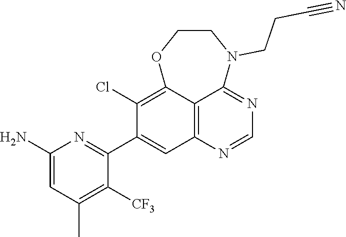 [SMILES: Cc1cc(N)nc(-c2cc3ncnc4c3c(c2Cl)OCCN4CCC#N)c1C(F)(F)F]

Under nitrogen, a solution of 3-(8-chloro-9-(4,4,5,5-tetramethyl-1,3,2-dioxaborolan-2-yl)-5,6-dihydro-4H-[1,4]oxazepino[5,6,7-de]quinazolin-4-yl)propanenitrile (800 mg, crude), bis(triphenylphosphine)palladium(11) chloride (2.7 mg, 0.01 mmol), potassium fluoride (6.8 mg, 0.12 mmol) and 6-bromo-4-methyl-5-(trifluoromethyl)pyridin-2-amine (135.0 mg, 0.53 mmol) in acetonitrile (10.0 mL) and water (2.0 mL) was added at 80° C. for 3 hours. After completion, the reaction mixture was diluted with ethyl acetate. The resulting solution was washed with water. The organic layer was dried over anhydrous sodium sulfate and concentrated under vacuum. The residue was purified by flash chromatography on silica gel eluting with dichloromethane methanol (10/1) to afford crude. The crude product was purified by Prep-HPLC with the following conditions: Column: XBridge Prep OBD C18 Column, 30×150 mm 5 um; Mobile Phase A: Water (10 MMOL/L NH4HCO3), Mobile Phase B: ACN; Flow rate: 60 mL/min; Gradient: 26 B to 56 B in 10 min; 254 nm; RT1: 9.50; to afford 3-(9-(6-amino-4-methyl-3-(trifluoromethyl)pyridin-2-yl)-8-chloro-5,6-dihydro-4H-[1,4]oxazepino[5,6,7-de]quinazolin-4-yl)propanenitrile (48.9 mg, 0.11 mmol, 20.6% yield). LC-MS: (ESI, m/z): 449.1 [M+H]⁺.

Example 13: ¹H NMR (300 MHz, DMSO-d₆, ppm) δ8.47 (s, 1H), 7.19 (s, 1H), 6.75 (s, 2H), 6.48-6.41 (m, 1H), 4.64 (d, J=12.6, 5.1, 2.6 Hz, 2H), 4.22-3.93 (m, 4H), 2.98 (t, J=6.7 Hz, 2H), 2.35 (d, J=2.1 Hz, 3H)

Example 14: 6-(8-chloro-4-(2-(oxetan-3-yl)ethyl)-5,6-dihydro-4H-[1,4]oxazepino[5,6,7-de]quinazolin-9-yl)-4-methyl-5-(trifluoromethyl)pyridin-2-amine

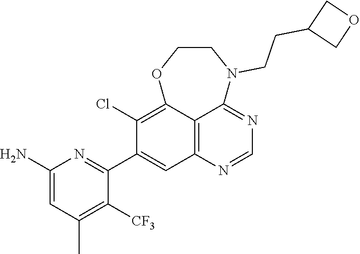 [SMILES: Cc1cc(N)nc(-c2cc3ncnc4c3c(c2Cl)OCCN4CCC2COC2)c1C(F)(F)F]

### Synthetic Route

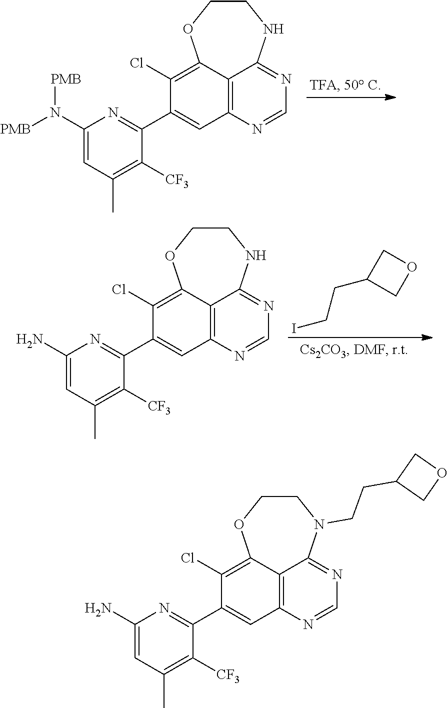

Step 1: 6-(8-chloro-5,6-dihydro-4H-[1,4]oxazepino[5,6,7-de]quinazolin-9-yl)-4-methyl-5-(trifluoromethyl)pyridin-2-amine

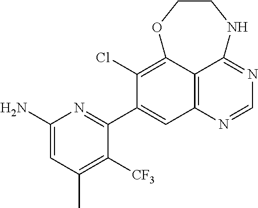 [SMILES: Cc1cc(N)nc(-c2cc3ncnc4c3c(c2Cl)OCCN4)c1C(F)(F)F]

A solution of 6-(8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.31 mmol) in 2,2,2-trifluoroacetic acid (2 mL) was stirred at 50° C. for 2 hours. The solvent was concentrated under vacuum. The residue was purified by flash chromatography on reverse phase with acetonitrile/water (50%) to afford 6-(8-chloro-5,6-dihydro-4H-[1,4]oxazepino[5,6,7-de]quinazolin-9-yl)-4-methyl-5-(trifluoromethyl)pyridin-2-amine (80.0 mg, 0.20 mmol, 64.3% yield) as a white solid. LC-MS: (ESI, m/z): 395.7 [M+H]⁺.

Step 2: 6-(8-chloro-4-(2-(oxetan-3-yl)ethyl)-5,6-dihydro-4H-[1,4]oxazepino[5,6,7-de]quinazolin-9-yl)-4-methyl-5-(trifluoromethyl)pyridin-2-amine

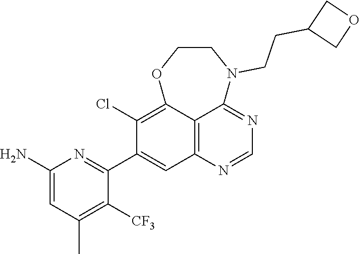 [SMILES: Cc1cc(N)nc(-c2cc3ncnc4c3c(c2Cl)OCCN4CCC2COC2)c1C(F)(F)F]

A solution of 6-(8-chloro-5,6-dihydro-4H-[1,4]oxazepino[5,6,7-de]quinazolin-9-yl)-4-methyl-5-(trifluoromethyl)pyridin-2-amine (50.0 mg, 0.12 mmol) and cesium carbonate (82.3 mg, 0.24 mmol) in N,N-dimethylformamide (1 mL) was stirred at 25° C. for 5 minutes. Then 3-(2-iodoethyl)oxetane (53.5 mg, 0.24 mmol) was added and stirred at 25° C. for 3 hours. After completion, The solvent was concentrated under vacuum, The crude product was purified by Prep-HPLC with the following conditions: Column: XBridge Prep OBD C18 Column, 30×150 mm 5 um; Mobile Phase A: Water (10 MMOL/L NH4HCO3), Mobile Phase B: ACN; Flow rate: 60 mL/min; Gradient: 23 B to 53 B in 7 min; 254 nm; RT1: 6.5 to afford 6-(8-chloro-4-(2-(oxetan-3-yl)ethyl)-5,6-dihydro-4H-[1,4]oxazepino[5,6,7-de]quinazolin-9-yl)-4-methyl-5-(trifluoromethyl)pyridin-2-amine (26.5 mg, 0.05 mmol, 43.7% yield). (ESI, m/z): 480.1 [M+H]⁺.

Example 14: ¹H NMR (400 MHz, DMSO-d₆, ppm) δ 8.40 (s, 1H), 7.14 (s, 1H), 6.76 (s, 2H), 6.48-6.43 (m, 1H), 4.69-4.53 (m, 4H), 4.32 (d, J=6.0, 1.6 Hz, 2H), 4.00-3.86 (m, 2H), 3.90-3.69 (m, 2H), 2.98 (d, J=8.1, 6.3 Hz, 1H), 2.36 (d, J=2.1 Hz, 3H), 2.10-1.99 (m, 2H).

Example 15: 6-(4-(1-(1H-imidazol-5-yl)ethyl)-8-chloro-5,6-dihydro-4H-[1,4]oxazepino[5,6,7-de]quinazolin-9-yl)-4-methyl-5-(trifluoromethyl)pyridin-2-amine

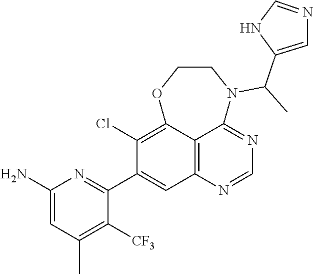 [SMILES: Cc1cc(N)nc(-c2cc3ncnc4c3c(c2Cl)OCCN4C(C)c2cnc[nH]2)c1C(F)(F)F]

### Synthetic Route

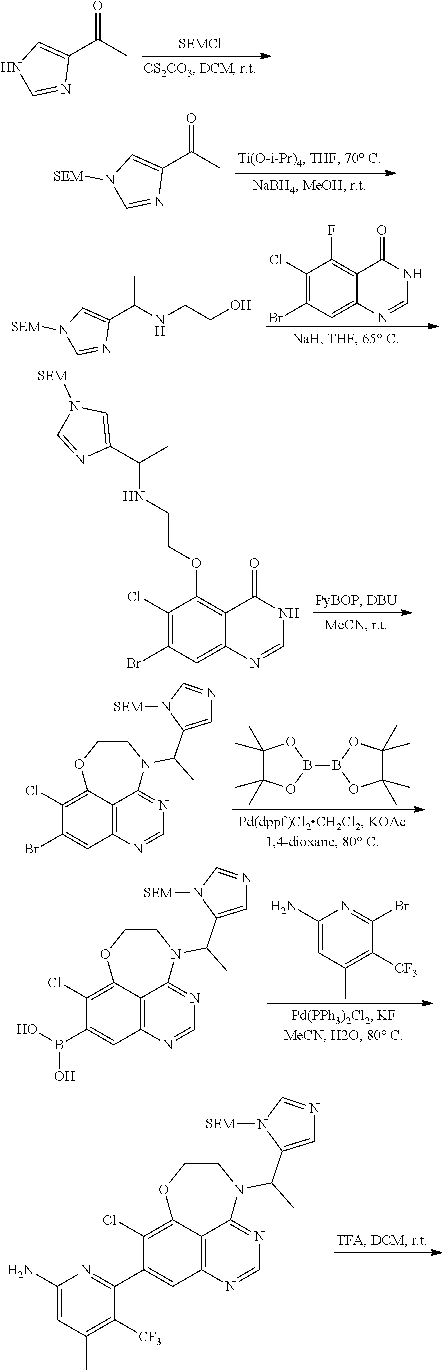

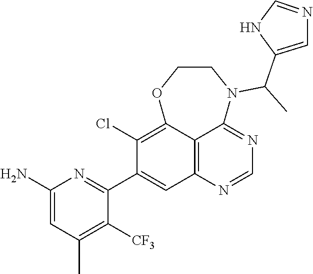 [SMILES: Cc1cc(N)nc(-c2cc3ncnc4c3c(c2Cl)OCCN4C(C)c2cnc[nH]2)c1C(F)(F)F]

Step 1: 1-(1-((2-(trimethylsilyl)ethoxy)methyl)-1H-imidazol-4-yl)ethan-1-one

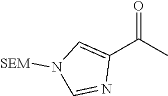 [SMILES: CC(=O)c1cn(COCC[Si](C)(C)C)cn1]

A solution of 1-(1H-imidazol-4-yl)ethanone (4.00 g, 36.30 mmol) and cesium carbonate (23.60 g, 72.60 mmol) in dichloromethane (50 mL) was stirred at 25° C. for 5 minute. Then 2-(trimethylsilyl)ethoxymethyl chloride (18.10 g, 108.90 mmol) was added and stirred at 25° C. for 12 hours. After completion, after filtration, the filtrate was concentrated under vacuum. The residue was purified by flash chromatography on silica gel eluting with dichloromethane/methanol (10:1) to afford 1-(1-((2-(trimethylsilyl)ethoxy)methyl)-1H-imidazol-4-yl)ethan-1-one (3.60 g, 14.93 mmol, 41.2% yield) as a yellow solid. LC-MS: (ESI, m/z): 241.4 [M+H]⁺.

Step 2: 2-((1-(1-((2-(trimethylsilyl)ethoxy)methyl)-1H-imidazol-4-yl)ethyl)amino)ethan-1-ol

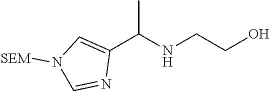 [SMILES: CC(NCCO)c1cn(COCC[Si](C)(C)C)cn1]

A solution of 1-(1-((2-(trimethylsilyl)ethoxy)methyl)-1H-imidazol-4-yl)ethan-1-one (3.60 g, 14.93 mmol) and 2-aminoethanol (1.8 mL, 29.8 mmol) and titanium isopropoxide (5.32 g, 18.76 mmol) in tetrahydrofuran (30 mL) was stirred at 70° C. for 12 hours. Then sodium borohydride (0.71 g, 18.76 mmol) was added and stirred at 25° C. for 1 hour. After completion, the reaction mixture was diluted with water t, extracted with ethyl acetate and the organic layers were combined. The organic layer was dried over anhydrous sodium sulfate and concentrated under vacuum. The residue was purified by flash chromatography on silica gel eluting with dichloromethane/methanol (1/1) to afford 2-((1-(1-((2-(trimethylsilyl)ethoxy)methyl)-1H-imidazol-4-yl)ethyl)amino)ethan-1-ol (1.00 g, 3.50 mmol, 56.1% yield) as a yellow oil. LC-MS: (ESI, m/z): 286.5 [M+H]⁺.

Step 3: 7-bromo-6-chloro-5-(2-((1-(1-((2-(trimethylsilyl)ethoxy)methyl)-1H-imidazol-4-yl)ethyl)amino)ethoxy)quinazolin-4(3H)-one

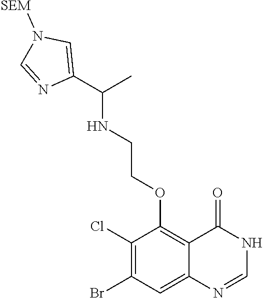 [SMILES: CC(NCCOc1c(Cl)c(Br)cc2nc[nH]c(=O)c12)c1cn(COCC[Si](C)(C)C)cn1]

A solution of 2-((1-(1-((2-(trimethylsilyl)ethoxy)methyl)-1H-imidazol-4-yl)ethyl)amino)ethan-1-ol (1.23 g, 4.30 mmol) in tetrahydrofuran (15 mL) was added sodium hydride (345.9 mg, 8.60 mmol, 60% purity) at 0° C. Then 7-bromo-6-chloro-5-fluoro-3H-quinazolin-4-one (600.0 mg, 2.10 mmol) was added and stirred at 0° C. for 5 minutes. The resulting solution was stirred for 2 hours at 65° C. After completion, the residue was dissolved with dichloromethane and the pH was adjusted to 7-8 with hydrochloric acid. The solvent was concentrated under vacuum. The residue was purified by flash chromatography reverse phase with acetonitrile/water (1/4) to afford 7-bromo-6-chloro-5-(2-((1-(1-((2-(trimethylsilyl)ethoxy)methyl)-1H-imidazol-4-yl)ethyl)amino)ethoxy)quinazolin-4(3H)-one (840.0 mg, 1.54 mmol, 71.6% yield) as a white solid. LC-MS: (ESI, m/z): 542.1 [M+H]⁺.

Step 4: 9-bromo-8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazoline

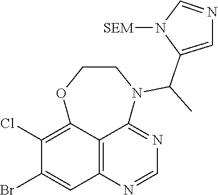 [SMILES: CC(c1cncn1COCC[Si](C)(C)C)N1CCOc2c(Cl)c(Br)cc3ncnc1c23]

A solution of 7-bromo-6-chloro-5-(2-((1-(1-((2-(trimethylsilyl)ethoxy)methyl)-1H-imidazol-4-yl)ethyl)amino)ethoxy)quinazolin-4(3H)-one (820.0 mg, 1.51 mmol) in acetonitrile (10 mL) was added 1,8-diazabicyclo[5.4.0]undec-7-ene (689.8 mg, 4.53 mmol) and benzotriazole-1-yl-oxytripyrrolidinophosphonium hexafluorophosphate (942.8 mg, 1.82 mmol) at 25° C. Then the solution was stirred at 25° C. for 2 hours. After completion, the reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford 9-bromo-8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazoline (520.0 mg, 0.99 mmol, 65.6% yield) as a yellow solid. LC-MS: (ESI, m/z): 524.9 [M+H]⁺.

Step 5: (8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazolin-9-yl)boronic acid

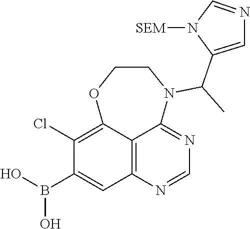 [SMILES: CC(c1cncn1COCC[Si](C)(C)C)N1CCOc2c(Cl)c(B(O)O)cc3ncnc1c23]

Under nitrogen, a solution of 9-bromo-8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazoline (300.0 mg, 0.53 mmol), potassium acetate (112.1 mg, 1.12 mmol), 1,1′-bis(diphenylphosphino)ferrocene-palladium(II)dichloride dichloromethane complex (46.67 mg, 0.06 mmol) and bis(pinacolato)diboron (435.4 mg, 1.73 mmol) in 1,4-dioxane (3 mL) was stirred at 80° C. for 2 hours. After completion, the solvent was concentrated under vacuum. The reaction mixture was diluted with dichloromethane. After filtration, the filtrate was concentrated under vacuum to afford the crude product (500 mg crude) which would be directly used in the next step without purification. LC-MS: (ESI, m/z): 489.8 [M+H]⁺.

Step 6: 6-(8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazolin-9-yl)-4-methyl-5-(trifluoromethyl)pyridin-2-amine

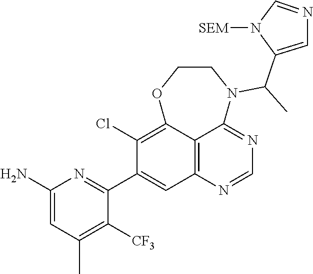 [SMILES: Cc1cc(N)nc(-c2cc3ncnc4c3c(c2Cl)OCCN4C(C)c2cncn2COCC[Si](C)(C)C)c1C(F)(F)F]

Under nitrogen, a solution of 6-bromo-4-methyl-5-(trifluoromethyl)pyridin-2-amine (160.0 mg, 0.6 mmol), bis(triphenylphosphine)palladium(II) chloride (44.0 mg, 0.06 mmol), potassium fluoride (72.9 mg, 1.22 mmol) and (8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazolin-9-yl)boronic acid (800.0 mg, crude) in acetonitrile (5 mL) and water (1 mL) was stirred at 80° C. for 2 hours. After completion, the reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford 6-(8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazolin-9-yl)-4-methyl-5-(trifluoromethyl)pyridin-2-amine (210.0 mg, 0.33 mmol, 54% yield) as a yellow solid. LC-MS: (ESI, m/z): 620.1 [M+H]⁺.

Step 7: 6-(4-(1-(1H-imidazol-5-yl)ethyl)-8-chloro-5,6-dihydro-4H-[1,4]oxazepino[5,6,7-de]quinazolin-9-yl)-4-methyl-5-(trifluoromethyl)pyridin-2-amine

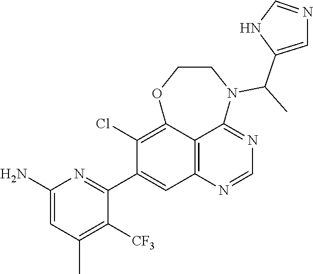 [SMILES: Cc1cc(N)nc(-c2cc3ncnc4c3c(c2Cl)OCCN4C(C)c2cnc[nH]2)c1C(F)(F)F]

A solution of 6-(8-chloro-4-(1-(1-((2-(trimethylsilyl)ethoxy)methyl)-1H-imidazol-5-yl)ethyl)-5,6-dihydro-4H-[1,4]oxazepino[5,6,7-de]quinazolin-9-yl)-4-methyl-5-(trifluoromethyl)pyridin-2-amine (200.0 mg, 0.31 mmol) in dichloromethane (1 mL) and trifluoroacetic acid (1 mL) was stirred at 25° C. 2 hours. After completion, the solvent was concentrated under vacuum. The reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford crude. The crude product was purified by Prep-HPLC with the following conditions: Column: Xselect CSH OBD Column 30*150 mm Sum, n; Mobile Phase A: Water (0.1% FA), Mobile Phase B: ACN; Flow rate: 60 mL/min; Gradient: 6 B to 19 B in 8 min; 254/220 nm; RT1: 7.15, 10.35 to afford 6-(4-(1-(1H-imidazol-5-yl)ethyl)-8-chloro-5,6-dihydro-4H-[1,4]oxazepino[5,6,7-de]quinazolin-9-yl)-4-methyl-5-(trifluoromethyl)pyridin-2-amine (9.4 mg, 0.01 mmol, 5.9% yield). LC-MS: (ESI, m/z): 490.1 [M+H]⁺.

Example 15: ¹H NMR (300 MHz, DMSO-d₆, ppm) δ 12.06 (s, 1H), 8.44 (s, 1H), 7.64 (s, 1H), 7.17 (d, J=6.5 Hz, 2H), 6.78 (s, 2H), 6.45 (d, 2H), 4.56-4.35 (m, 2H), 3.60 (d, 2H), 2.36 (d, J=2.3 Hz, 3H), 1.52 (d, J=7.0 Hz, 3H).

Example 16: 2-(9-(6-amino-4-methyl-3-(trifluoromethyl)pyridin-2-yl)-8-chloro-5,6-dihydro-4H-[1,4]oxazepino[5,6,7-de]quinazolin-4-yl)propan-1-ol

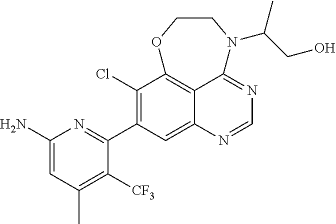 [SMILES: Cc1cc(N)nc(-c2cc3ncnc4c3c(c2Cl)OCCN4C(C)CO)c1C(F)(F)F]

### Synthetic Route

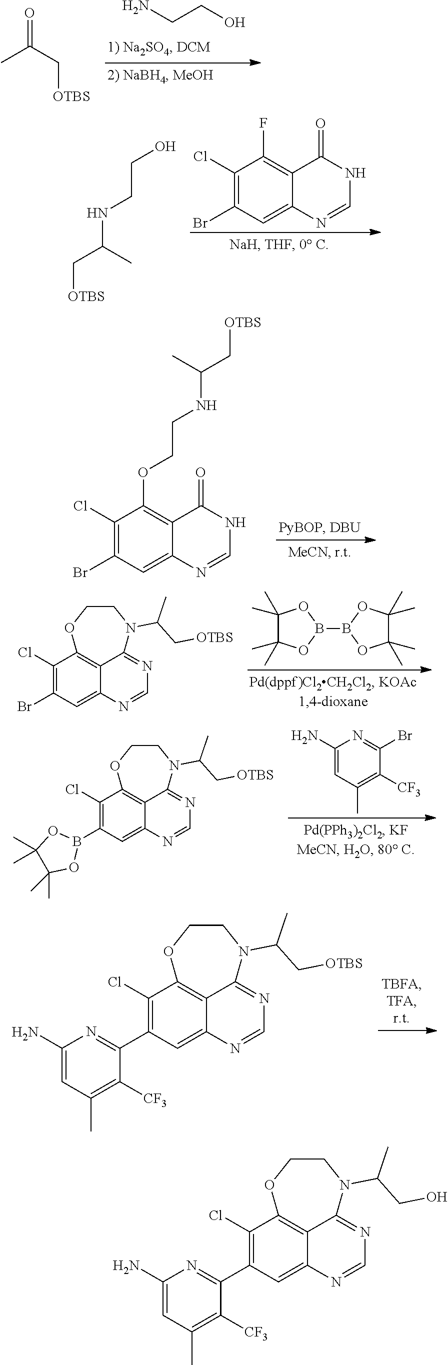

Step 1: 2-[[2-[tert-butyl(dimethyl)silyl]oxy-1-methyl-ethyl]amino]ethanol

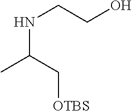

A solution of 1-(tert-butyldimethylsilyloxy)-2-propanone (4.50 g, 23.89 mmol) and sodium sulfate (6.79 g, 47.79 mmol) in dichloromethane (50 mL) was stirred at 25° C. for 5 minutes. Then 2-aminoethanol (1.46 g, 23.89 mmol) was added and stirred at 25° C. for 2 hours. After completion, the solvent was concentrated under vacuum. The crude product would be directly used in the next step without purification. Then the residue and sodium borohydride (0.81 g, 21.39 mmol) in methyl alcohol (0.5 mL) was stirred at 25° C. for 4 hours. After completion, the reaction mixture was adjusted to pH hydrochloric acid with 7-8. The solvent was concentrated under vacuum. The residue was purified by flash chromatography on silica gel eluting with dichloromethane/methanol (5/1) to afford 2-[[2-[tert-butyl(dimethyl)silyl]oxy-1-methyl-ethyl]amino]ethanol (2.9 g, 12.42 mmol, 63.9% yield) as a yellow oil.

Step 2: 7-bromo-5-(2-((1-((tert-butyldimethylsilyl)oxy)propan-2-yl)amino)ethoxy)-6-chloroquinazolin-4(3H)-one

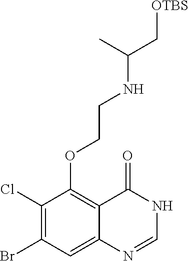 [SMILES: CC(CO[Si](C)(C)C(C)(C)C)NCCOc1c(Cl)c(Br)cc2nc[nH]c(=O)c12]

A solution of 2-[[2-[tert-butyl(dimethyl)silyl]oxy-1-methyl-ethyl]amino]ethanol (841.2 mg, 3.60 mmol) and sodium hydride (288.3 mg, 7.20 mmol, 60% purity) in tetrahydrofuran (5.0 mL) was stirred at 0° C. for 5 minutes. Then 7-bromo-6-chloro-5-fluoro-3H-quinazolin-4-one (500.0 mg, 1.80 mmol) was added and stirred at 0° C. for 2 hours. After completion, the residue was dissolved with dichloromethane and the pH was adjusted to 7-8 with hydrochloric acid. The solvent was concentrated under vacuum. The residue was purified by flash chromatography on silica gel eluting with acetonitrile/water (5:1) to afford 7-bromo-5-(2-((1-((tert-butyldimethylsilyl)oxy)propan-2-yl)amino)ethoxy)-6-chloroquinazolin-4(3H)-one (490.0 mg, 0.99 mmol, 55.4% yield) as a white solid. LC-MS: (ESI, m/z): 490.9 [M+H]⁺.

Step 3:9-bromo-4-(1-((tert-butyldimethylsilyl)oxy)propan-2-yl)-8-chloro-5,6-dihydro-4H-[1,4]oxazepino[5,6,7-de]quinazoline

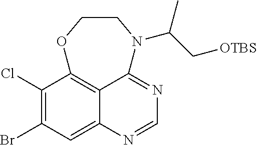 [SMILES: CC(CO[Si](C)(C)C(C)(C)C)N1CCOc2c(Cl)c(Br)cc3ncnc1c23]

A solution of 7-bromo-5-(2-((1-((tert-butyldimethylsilyl)oxy)propan-2-yl)amino)ethoxy)-6-chloroquinazolin-4(3H)-one (470.0 mg, 0.96 mmol) and 1,8-diazabicyclo[5.4.0]undec-7-ene (437.2 mg, 2.87 mmol) in acetonitrile (5.0 mL) was stirred at 25° C. for 5 minute. Then benzotriazole-1-yl-oxytripyrrolidinophosphonium hexafluorophosphate (597.7 mg, 1.15 mmol) was added and stirred at 25° C. for 2 hours. After completion, the reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford 9-bromo-4-(1-((tert-butyldimethylsilyl)oxy)propan-2-yl)-8-chloro-5,6-dihydro-4H-[1,4]oxazepino[5,6,7-de]quinazoline (340.0 mg, 0.71 mmol, 75.1% yield) as a yellow solid. LC-MS: (ESI, m/z): 472.9 [M+H]⁺.

Step 4: 4-(1-((ted-butyldimethylsilyl)oxy)propan-2-yl)-8-chloro-9-(4,4,5,5-tetramethyl-1,3,2-dioxaborolan-2-yl)-5,6-dihydro-4H-[1,4]oxazepino[5,6,7-de]quinazoline

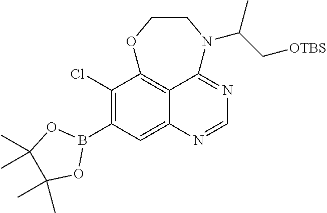 [SMILES: CC(CO[Si](C)(C)C(C)(C)C)N1CCOc2c(Cl)c(B3OC(C)(C)C(C)(C)O3)cc3ncnc1c23]

Under nitrogen, a solution of 9-bromo-4-(1-((tert-butyldimethylsilyl)oxy)propan-2-yl)-8-chloro-5,6-dihydro-4H-[1,4]oxazepino[5,6,7-de]quinazoline (340.0 mg, 0.72 mmol), bis(pinacolato)diboron (547.7 mg, 2.16 mmol), potassium acetate (141.1 mg, 1.4 mmol) and [1,1′-bis(diphenylphosphino)ferrocene]dichloropalladium(II) (59.4 mg, 0.07 mmol) in 1,4-dioxane (3.0 mL) was stirred at 80° C. for 4 hours. After completion, the solvent was concentrated under vacuum. The reaction mixture was diluted with dichloromethane. After filtration, the filtrate was concentrated under reduced pressure. The reaction mixture was diluted with petroleum ether. After filtration, the solid was the crude product (600 mg, crude) which would be directly used in the next step without purification. LC-MS: (ESI, m/z): 520.2 [M+H]⁺.

Step 5: 6-(4-(1-((tert-butyldimethylsilyl)oxy)propan-2-yl)-8-chloro-5,6-dihydro-4H-[1,4]oxazepino[5,6,7-de]quinazolin-9-yl)-4-methyl-5-(trifluoromethyl)pyridin-2-amine

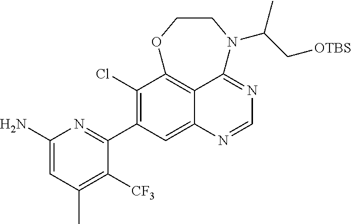 [SMILES: Cc1cc(N)nc(-c2cc3ncnc4c3c(c2Cl)OCCN4C(C)CO[Si](C)(C)C(C)(C)C)c1C(F)(F)F]

Under nitrogen, a solution of 4-(1-((tert-butyldimethylsilyl)oxy)propan-2-yl)-8-chloro-9-(4,4,5,5-tetramethyl-1,3,2-dioxaborolan-2-yl)-5,6-dihydro-4H-[1,4]oxazepino[5,6,7-de]quinazoline (600.0 mg, crude), 6-bromo-4-methyl-5-(trifluoromethyl)pyridin-2-amine (250.0 mg, 0.98 mmol), potassium fluoride (113.9 mg, 1.96 mmol) and bis(triphenylphosphine)palladium(II) chloride (68.8 mg, 0.1 mmol) in acetonitrile (0.5 mL) and water (0.1 mL) was stirred at 80° C. for 4 hours. After completion, the reaction mixture was diluted with ethyl acetate. The resulting solution was washed with water, dried over anhydrous sodium sulfate and concentrated under vacuum. The residue was purified by flash chromatography on silica gel eluting with petroleum ether/ethyl acetate (1/10) to afford 6-(4-(1-((tert-butyldimethylsilyl)oxy)propan-2-yl)-8-chloro-5,6-dihydro-4H-[1,4]oxazepino[5,6,7-de]quinazolin-9-yl)-4-methyl-5-(trifluoromethyl)pyridin-2-amine (280.0 mg, 0.49 mmol, 50.3% yield) as a yellow solid. LC-MS: (ESI, m/z): 568.1 [M+H]⁺.

Step 6: 2-(9-(6-amino-4-methyl-3-(trifluoromethyl)pyridin-2-yl)-8-chloro-5,6-dihydro-4H-[1,4]oxazepino[5,6,7-de]quinazolin-4-yl)propan-1-ol

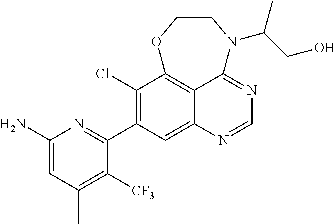 [SMILES: Cc1cc(N)nc(-c2cc3ncnc4c3c(c2Cl)OCCN4C(C)CO)c1C(F)(F)F]

A solution of 6-(4-(1-((tert-butyldimethylsilyl)oxy)propan-2-yl)-8-chloro-5,6-dihydro-4H-[1,4]oxazepino[5,6,7-de]quinazolin-9-yl)-4-methyl-5-(trifluoromethyl)pyridin-2-amine (240.0 mg, 0.43 mmol) and tetrabutylammonium fluoride (0.84 mL, 0.86 mmol) in tetrahydrofuran (3.0 mL) was stirred at 25° C. for 8 hours. After completion, the reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (5/1) to afford crude. The crude product was purified by Prep-HPLC with the following conditions: Column: XBridge Prep OBD C18 Column, 30×150 mm 5 um; Mobile Phase A: Water (10 mmol/L NH₄HCO₃), Mobile Phase B: ACN: Flow rate: 60 mL/min; Gradient: 23 B to 53 B in 9 min; 254 nm; RT1: 8.5 to afford 2-(9-(6-amino-4-methyl-3-(trifluoromethyl)pyridin-2-yl)-8-chloro-5,6-dihydro-4H-[1,4]oxazepino[5,6,7-de]quinazolin-4-yl)propan-1-ol (56.0 mg, 0.12 mmol, 29.2% yield). LC-MS: (ESI, m/z): 454.1 [M+H]⁺.

Example 16: ¹H NMR (300 MHz, DMSO-d₆, ppm) δ 8.37 (d, J=0.9 Hz, 1H), 7.12 (s, 1H), 6.75 (s, 2H), 6.46-6.39 (m, 1H), 5.33-5.16 (m, 1H), 4.84 (d, J=5.4, 1.9 Hz, 1H), 4.73-4.44 (m, 2H), 3.87-3.64 (m, 2H), 3.67-3.48 (m, 2H), 2.37-2.29 (m, 3H), 1.16 (d, J=6.8, 1.9 Hz, 3H).

Example 17: (R)-6-(4-(1-(5-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

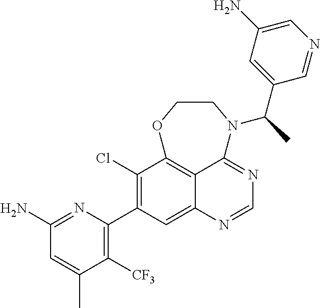 [SMILES: Cc1cc(N)nc(-c2cc3ncnc4c3c(c2Cl)OCCN4[C@H](C)c2cncc(N)c2)c1C(F)(F)F]

### Synthetic Route

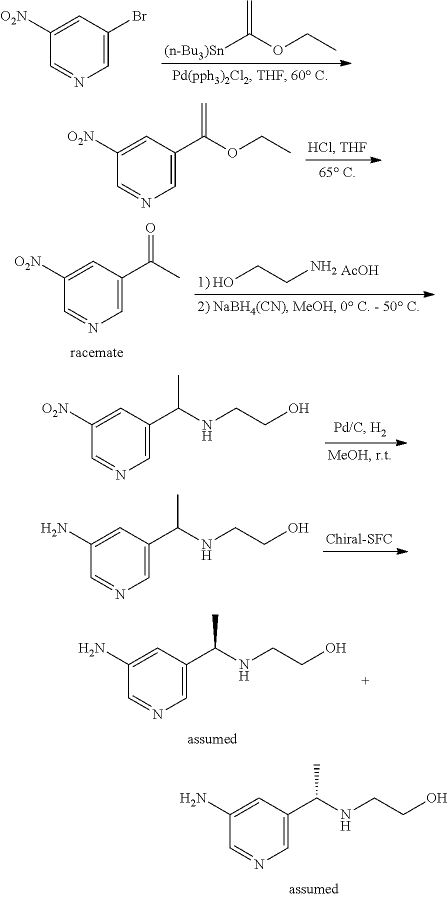

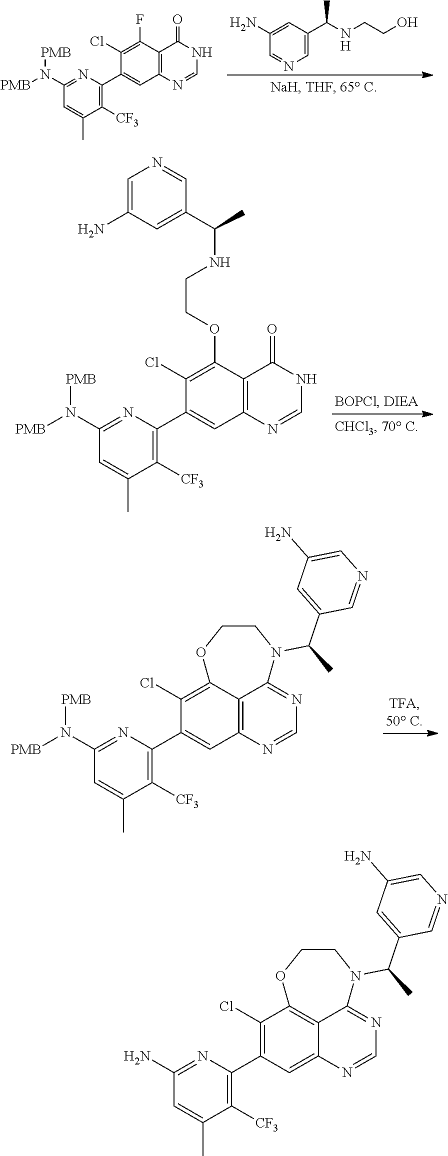

Step 1: 3-(1-ethoxyvinyl)-5-nitropyridine

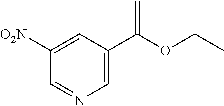

A solution of 3-bromo-5-nitropyridine (25.00 g, 123.16 mmol), tributyl(1-ethoxyvinyl)stannane (88.96 g, 246.32 mmol) and bis(triphenylphosphine)palladium(II) chloride (8.65 g, 12.32 mmol) in tetrahydrofuran (500 mL) was stirred at 60° C. for 6 hours. After completion, the solvent was concentrated under vacuum. The residue was purified by flash chromatography on silica gel eluting with ethyl acetate/petroleum ether (1/10) to afford 3-(1-ethoxyvinyl)-5-nitropyridine (130 g, 66.94 mmol, 54.4% yield) as a yellow solid. LC-MS: (ESI, m/z): 195.0 [M+H]⁺

Step 2: 3-(1-ethoxyvinyl)-5-nitropyridine

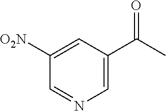

A solution of 3-(1-ethoxyvinyl)-5-nitro-pyridine (13.70 g, 70.55 mmol) and Hydrochloric acid (25.72 g, 705.49 mmol) in tetrahydrofuran (150 mL) was stirred at 50° C. for 3 hours. After completion, the solvent was concentrated under vacuum. The residue was purified by flash chromatography on silica gel eluting with ethyl acetate/petroleum ether (1/10) to afford 3-(1-ethoxyvinyl)-5-nitropyridine (10.00 g, 60.19 mmol, 85.3% yield) as a yellow solid. LC-MS: (ESI, m/z): 167.0 [M+H]⁺

Step 3: 2-((1-(5-nitropyridin-3-yl)ethyl)amino)ethan-1-ol

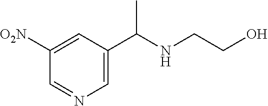

A solution of 2-aminoethanol (8.7 ml, 144.47 mmol), 1-(5-nitro-3-pyridyl)ethanone (20.0 g, 120.39 mmol) and acetic acid (0.69 ml, 12.04 mmol) in methyl alcohol (50 ml) was stirred at room temperature for 2 hours. Then sodium cyanoborohydride (22.70 g, 361.16 mmol) was added and stirred at 0° C. for 3 hours. After completion, the solvent was concentrated under vacuum. The residue was purified by flash chromatography on silica gel eluting with methanol/dichloromethane (1:50) to afford 2-((1-(5-nitropyridin-3-yl)ethyl)amino)ethan-1-ol (12.10 g, 57.28 mmol, 47.6% yield) as a yellow solid. LC-MS: (ESI, m/z): 212.1 [M+H]⁺

Step 4: (R)-2-((1-(5-aminopyridin-3-yl)ethyl)amino)ethan-1-01 and (S)-2-((1-(5-aminopyridin-3-yl)ethyl)amino)ethan-1-ol

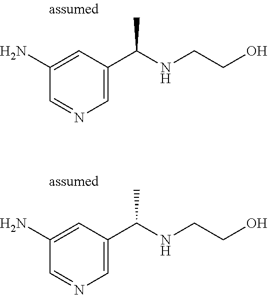

Under hydrogen, a solution of 2-((1-(5-nitropyridin-3-yl)ethyl)amino)ethan-1-ol (7.00 g, 33.14 mmol) and heavy distillate (10.00 g, 331.41 mmol) in Ethyl acetate (100 mL) was stirred for 3 hours at room temperature. After completion, the solvent was filtered, the filtrate was concentrated under vacuum. The residue was purified by flash chromatography on silica gel eluting with methanol/dichloromethane (1/10) to afford 4.2 g crude. The product was purified by Chiral-Prep-HPLC with the following conditions: Column: EnantioPak A1-5, 2.12*25 cm, 5 μm; Mobile Phase A: C02, Mobile Phase B: MEOH (0.1% 2M NH3-MEOH); Flow rate: 50 mL/min; Gradient: isocratic 17% B; Column Temperature (° C.): 35; Back Pressure (bar): 100: Wave Length: 220 nm; RT1(min): 4.54: RT2(min): 6.02; Sample Solvent: MeOH—Preparative; Injection Volume: 0.4 mL; Number Of Runs: 150 to afford (R)-2-((1-(5-aminopyridin-3-yl)ethyl)amino)ethan-1-01(1.70 g, 9.34 mmol, 28.2% yield) as a yellow oil and (S)-2-((1-(5-aminopyridin-3-yl)ethyl)amino)ethan-1-ol (1.50 g, 8.24 mmol, 24.8% yield) as a yellow oil.

Step 5: (R)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one

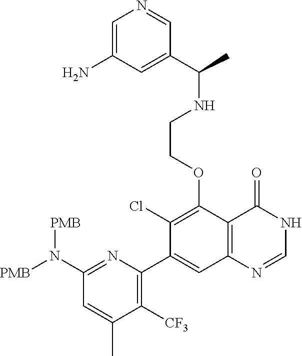

A solution of (R)-2-((1-(5-aminopyridin-3-yl)ethyl)amino)ethan-1-ol (106.4 mg, 0.58 mmol) and sodium hydride (58.7 mg, 1.44 mmol, 60% purity) in tetrahydrofuran (3.0 mL) was stirred at 0° C. for 5 minutes. Then 7-(6-(bis(4-methoxybenzyl)amino)-4-methyl-3-(trifluoromethyl)pyridin-2-yl)-6-chloro-5-fluoroquinazolin-4(3H)-one (300.0 mg, 0.58 mmol) was added and stirred at 65° C. for 1 hour. After completion, the residue was dissolved with dichloromethane and the pH was adjusted to 7-8 with 1N hydrochloric acid. The solvent was concentrated under vacuum. The residue was purified by flash chromatography on silica gel eluting with dichloromethane/methanol (10/1) to afford (R)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (280.0 mg, 0.36 mmol, 73.9% yield) as a white solid. LC-MS: (ESI, m/z): 774.2 [M+H]⁺.

Step 6: (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

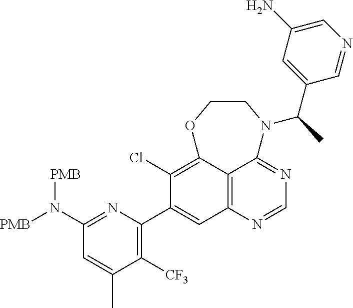

A solution of (R)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (270.0 mg, 0.35 mmol) and N,N-diisopropylethylamine (136.0 mg, 1.05 mmol), bis(2-oxo-3-oxazolidinyl)phosphinic chloride (115.4 mg, 0.41 mmol) in chloroform (3.0 mL) was stirred at 70° C. for 1 hour. After completion, the solvent was concentrated under vacuum. The reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (190.0 mg, 0.25 mmol, 72% yield) as a yellow solid. LC-MS: (ESI, m/z): 756.2 [M+H]⁺.

Step 7: (R)-6-(4-(1-(5-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

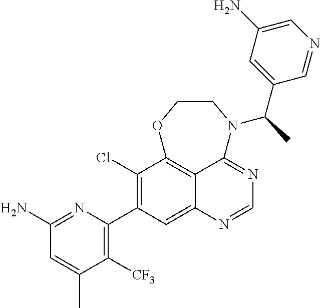 [SMILES: Cc1cc(N)nc(-c2cc3ncnc4c3c(c2Cl)OCCN4[C@H](C)c2cncc(N)c2)c1C(F)(F)F]

A solution of (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (180.0 mg, 0.24 mmol) in trifluoroacetic acid (2.0 mL) was stirred at 25° C. for 0.5 hours. After completion, the residue was dissolved with dichloromethane and the pH was adjusted to 7-8 with N,N-diisopropylethylamine. The solvent was concentrated under vacuum. The residue was purified by flash chromatography on reverse phase with acetonitrile/water (1/1) to afford crude. The crude product was purified by Prep-HPLC with the following conditions: Column: Xselect CSH OBD Column 30*150 mm Sum, n; Mobile Phase A: Water (0.1% FA), Mobile Phase B: ACN; Flow rate: 60 mL/min; Gradient: 11% B to 27% B in 8 min, 27% B; Wave Length: 254/220 nm; RT1(min): 6.12 to afford (R)-6-(4-(1-(5-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine (50.1 mg, 0.09 mmol, 40.8% yield). LC-MS: (ESI, m/z): 516.1 [M+H]⁺.

Example 17: ¹H NMR (300 MHz, DMSO-d6) δ 8.47 (d, J=1.7 Hz, 1H), 7.93-7.73 (m, 2H), 7.19 (d, J=1.2 Hz, 1H), 6.91 (d, J=9.8 Hz, 1H), 6.76 (s, 2H), 6.63-6.49 (m, 1H), 6.45 (s, 1H), 5.38 (s, 1H), 4.64-4.36 (m, 2H), 3.70 (dt, J=15.7, 7.7 Hz, 1H), 3.56-3.38 (m, 2H), 2.35 (d, J=2.3 Hz, 3H), 1.59 (dd, J=7.1, 2.3 Hz, 3H).

Example 18: (S)-6-(4-(1-(2-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

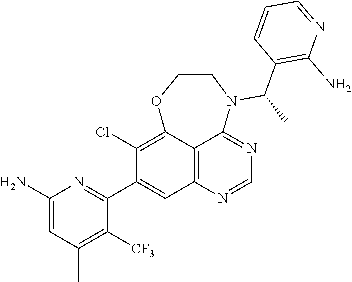 [SMILES: Cc1cc(N)nc(-c2cc3ncnc4c3c(c2Cl)OCCN4[C@@H](C)c2cccnc2N)c1C(F)(F)F]

### Synthetic Route

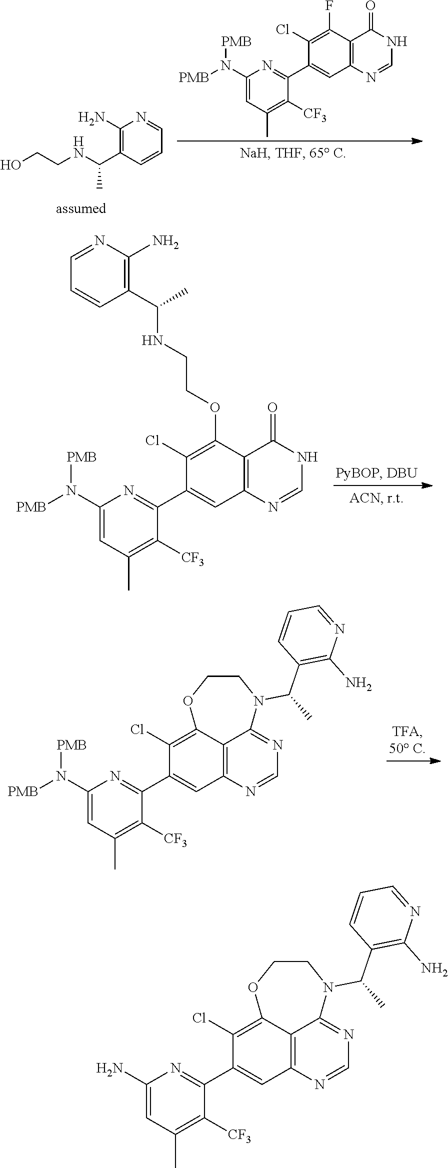

Step 1: (S)-5-(2-((1-(2-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one

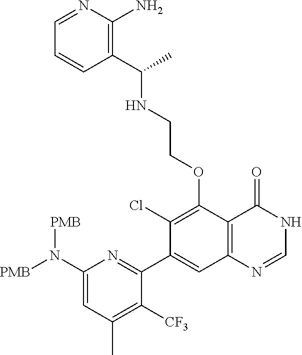

A solution of (S)-2-((1-(2-aminopyridin-3-yl)ethyl)amino)ethan-1-ol (266.0 mg, 1.45 mmol) and sodium hydride (97.8 mg, 2.40 mmol, 60% purity) in tetrahydrofuran (3.0 mL) was stirred at 0° C. for 5 minutes. Then 7-(6-(bis(4-methoxybenzyl)amino)-4-methyl-3-(trifluoromethyl)pyridin-2-yl)-6-chloro-5-fluoroquinazolin-4(3H)-one (300.0 mg, 0.59 mmol) was added and stirred at 65° C. for 2 hours. After completion, the residue was dissolved with dichloromethane and the pH was adjusted to 7-8 with hydrochloric acid. The solvent was concentrated under vacuum. The residue was purified by flash chromatography on silica gel eluting with dichloromethane/methanol (10:1) to afford (S)-5-(2-((1-(2-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (310.0 mg, 0.40 mmol, 81.8% yield) as a white solid. LC-MS: (ESI, m/z): 774.2 [M+H]⁺.

Step 2: (S)-6-(4-(1-(2-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

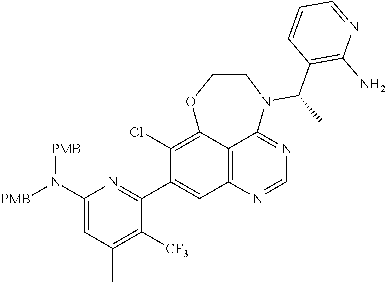

A solution of (S)-5-(2-((1-(2-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (310.0 mg, 0.4 mmol) and 1,8-diazabicyclo[5.4.0]undec-7-ene (182.8 mg, 1.23 mmol) in chloroform (3.0 mL) was stirred at 25° C. for 5 minute. Then benzotriazole-1-yl-oxytripyrrolidinophosphonium hexafluorophosphate (250.0 mg, 0.45 mmol) was added and stirred at 25° C. for 2 hours. After completion, the reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford (S)-6-(4-(1-(2-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (160.0 mg, 0.21 mmol, 52.8% yield) as a yellow solid. LC-MS: (ESI, m/z): 756.2 [M+H]⁺.

Step 3: (S)-6-(4-(1-(2-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine

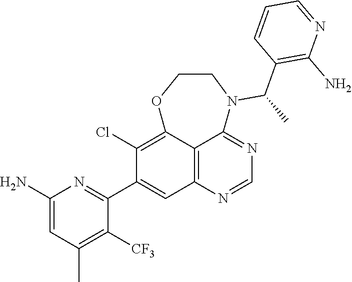 [SMILES: Cc1cc(N)nc(-c2cc3ncnc4c3c(c2Cl)OCCN4[C@@H](C)c2cccnc2N)c1C(F)(F)F]

A solution of (S)-6-(4-(1-(2-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (160.0 mg, 0.25 mmol) in trifluoroacetic acid (2.0 mL) was stirred at 50° C. for 6 hours. After completion, the residue was dissolved with dichloromethane and the pH was adjusted to 7-8 with N,N-diisopropylethylamine. The solvent was concentrated under vacuum. The residue was purified by flash chromatography on silica gel eluting with dichloromethane/methanol (1/1) to afford crude product. The crude product was purified by Prep-HPLC with the following conditions: Column: XBridge Prep OBD C18 Column, 19*250 mm, 5 um; Mobile Phase A: Water (10 MMOL/L NH4HCO3), Mobile Phase B: ACN; Flow rate: 25 mL/min; Gradient: 48 B to 60 B in 7 min; 254 nm; RT1: 6.57 to afford (S)-6-(4-(1-(2-aminopyridin-3-yl)ethyl)-8-chloro-5,6-dihydro-4H-[1,4]oxazepino[5,6,7-de]quinazolin-9-yl)-4-methyl-5-(trifluoromethyl)pyridin-2-amine (51.8 mg, 0.10 mmol, 47.5% yield). LC-MS: (ESI, m/z): 516.2 [M+H]⁺.

Example 18: ¹H NMR (300 MHz, DMSO-d₆, ppm) δ8.39 (s, 1H), 7.88-7.79 (m, 1H), 7.52 (d, J=7.4 Hz, 1H), 7.06 (s, 1H), 6.64 (s, 2H), 6.59-6.49 (m, 1H), 6.36-6.30 (m, 2H), 5.65 (d, J=21.6 Hz, 2H), 4.50-4.31 (m, 1H), 4.25-4.11 (m, 1H), 3.64-3.47 (m, 1H), 3.34-3.25 (m, 1H), 2.23 (d, J=2.3 Hz, 3H), 1.43 (d, J=6.0 Hz, 3H)

Example 19: (S)-2-(9-(6-amino-4-methyl-3-(trifluoromethyl)pyridin-2-yl)-4-((2-aminopyridin-3-yl)methyl)-8-chloro-5,6-dihydro-4H-[1,4]oxazepino[5,6,7-de]quinazolin-5-yl)acetonitrile

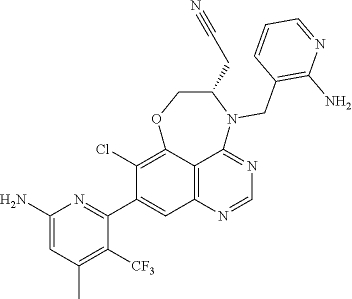 [SMILES: Cc1cc(N)nc(-c2cc3ncnc4c3c(c2Cl)OC[C@H](CC#N)N4Cc2cccnc2N)c1C(F)(F)F]

### Synthetic Route

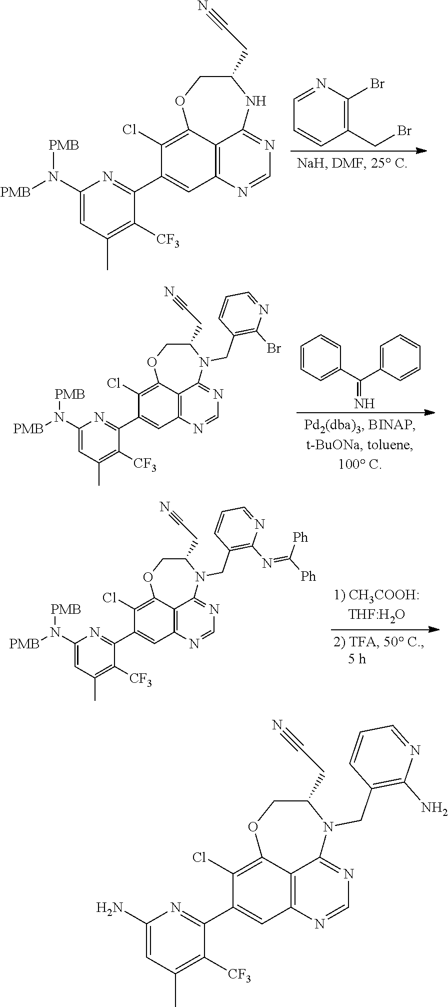

Step 1: (S)-2-(9-(6-(bis(4-methoxybenzyl)amino)-4-methyl-3-(trifluoromethyl)pyridin-2-yl)-4-((2-bromopyridin-3-yl)methyl)-8-chloro-5,6-dihydro-4H-[1,4]oxazepino[5,6,7-de]quinazolin-5-yl)acetonitrile

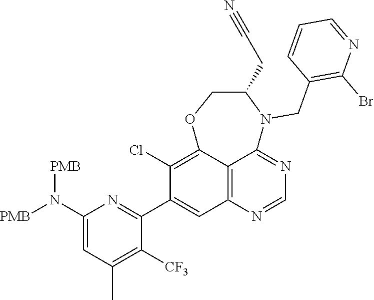

A solution of (S)-2-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-5-yl)acetonitrile (300.0 mg, 0.44 mmol) and sodium hydride (35.6 mg, 0.88 mmol, 60% purity) in N,N-dimethylformamide (3.0 mL) was stirred at 0° C. for 5 minutes. Then 2-bromo-3-(bromomethyl)pyridine (167.2 mg, 0.33 mmol) was added and stirred at 25° C. for 0.5 hours. After completion, the residue was dissolved with dichloromethane and the pH was adjusted to 7-8 with hydrochloric acid. The solvent was concentrated under vacuum. The residue was purified by flash chromatography on silica gel eluting with dichloromethane/methanol (10/1) to afford crude product. The crude product was purified by Prep-HPLC with the following conditions: Column: Xselect CSH OBD Column 30*150 mm Sum, n; Mobile Phase A: Water (0.1% FA), Mobile Phase B: ACN; Flow rate: 60 mL/min; Gradient: 65% B to 83% B in 10 min, 83% B; Wave Length: 254 nm; RT1(min): 7.55 to afford (S)-2-(9-(6-(bis(4-methoxybenzyl)amino)-4-methyl-3-(trifluoromethyl)pyridin-2-yl)-4-((2-bromopyridin-3-yl)methyl)-8-chloro-5,6-dihydro-4H-[1,4]oxazepino[5,6,7-de]quinazolin-5-yl)acetonitrile (170.0 mg, 0.55 mmol, 60% yield) as a white solid. LC-MS: (ESI, m/z): 845.1 [M+H]⁺.

Step 2: (S)-2-(9-(6-(bis(4-methoxybenzyl)amino)-4-methyl-3-(trifluoromethyl)pyridin-2-yl)-8-chloro-4-((2-((diphenylmethylene)amino)pyridin-3-yl)methyl)-5,6-dihydro-4H-[1,4]oxazepino[5,6,7-de]quinazolin-5-yl)acetonitrile

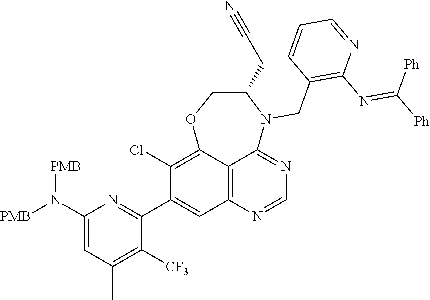

Under nitrogen, a solution of (S)-2-(9-(6-(bis(4-methoxybenzyl)amino)-4-methyl-3-(trifluoromethyl)pyridin-2-yl)-4-((2-bromopyridin-3-yl)methyl)-8-chloro-5,6-dihydro-4H-[1,4]oxazepino[5,6,7-de]quinazolin-5-yl)acetonitrile (150.0 mg, 0.18 mmol), diphenylmethanimine (0.04 mL, 0.27 mmol), 1.1′-binaphthyl-2.2′-diphenyl phosphine (22.1 mg, 0.04 mmol) and tris(dibenzylideneacetone)dipalladium (16.2 mg, 0.02 mmol) in toluene (3 mL) was added sodium tert-butoxide (34.1 mg, 0.35 mmol) at 100° C. The resulting solution was stirred for 1 h at 100° C. After completion, the solvent was concentrated under vacuum. The reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford (S)-2-(9-(6-(bis(4-methoxybenzyl)amino)-4-methyl-3-(trifluoromethyl)pyridin-2-yl)-8-chloro-4-((2-((diphenylmethylene)amino)pyridin-3-yl)methyl)-5,6-dihydro-4H-[1,4]oxazepino[5,6,7-de]quinazolin-5-yl)acetonitrile (65.0 mg, 0.15 mmol, 50.1% yield) as a white solid. LC-MS: (ESI, m/z): 945.4 [M+H]⁺.

Step 3: (S)-2-(9-(6-amino-4-methyl-3-(trifluoromethyl)pyridin-2-yl)-4-((2-aminopyridin-3-yl)methyl)-8-chloro-5,6-dihydro-4H-[1,4]oxazepino[5,6,7-de]quinazolin-5-yl)acetonitrile

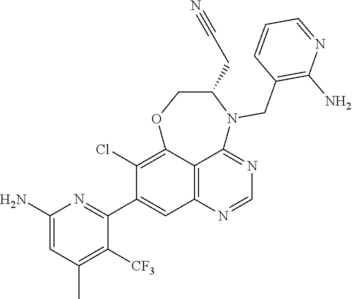 [SMILES: Cc1cc(N)nc(-c2cc3ncnc4c3c(c2Cl)OC[C@H](CC#N)N4Cc2cccnc2N)c1C(F)(F)F]

A solution of (S)-2-(9-(6-(bis(4-methoxybenzyl)amino)-4-methyl-3-(trifluoromethyl)pyridin-2-yl)-8-chloro-4-((2-((diphenylmethylene)amino)pyridin-3-yl)methyl)-5,6-dihydro-4H-[1,4]oxazepino[5,6,7-de]quinazolin-5-yl)acetonitrile (90.0 mg, 0.10 mmol) in acetic acid (0.5 mL), tetrahydrofuran (0.5 mL) and water (0.1 mL) was stirred at 50° C. for 1.5 hours. After completion, the solvent was concentrated under vacuum. The crude product would be directly used in the next step without purification. The crude product in trifluoroacetic acid (0.5 mL) was stirred at 50° C. for 5 hours. After completion, the solvent was concentrated under vacuum. The residue was purified by flash chromatography on silica gel eluting with dichloromethane/methanol (5/1) to afford crude. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min: Gradient: 34% B to 64% B in 7 min, 64% B; Wave Length: 254 nm; RT1(min): 6.5 to afford (S)-2-(9-(6-amino-4-methyl-3-(trifluoromethyl)pyridin-2-yl)-4-((2-aminopyridin-3-yl)methyl)-8-chloro-5,6-dihydro-4H-[1,4]oxazepino[5,6,7-de]quinazolin-5-yl)acetonitrile (11.1 mg, 0.02 mmol, 21.6% yield). LC-MS: (ESI, m/z): 541.1 [M+H]⁺.

Example 19: ¹H NMR (300 MHz, DMSO-d₆, ppm) δ8.16 (d, J=3.4 Hz, 1H), 7.88 (d, J=4.9, 1.5 Hz, 1H), 7.39-7.28 (m, 1H), 6.96 (d, J=3.3 Hz, 1H), 6.75 (d, J=3.6 Hz, 2H), 6.58-6.47 (m, 1H), 6.43 (s, 1H), 5.97 (s, 2H), 5.11-4.87 (m, 2H), 4.58-4.44 (m, 1H), 4.33-4.15 (m, 2H), 2.96-2.82 (m, 1H), 2.81-2.67 (m, 1H), 2.34 (d, J=2.3 Hz, 3H).

Example 20: 6-(4-(1-(2-aminopyridin-3-yl)-2,2,2-trifluoroethyl)-8-chloro-5,6-dihydro-4H-[1,4]oxazepino[5,6,7-de]quinazolin-9-yl)-4-methyl-5-(trifluoromethyl)pyridin-2-amine

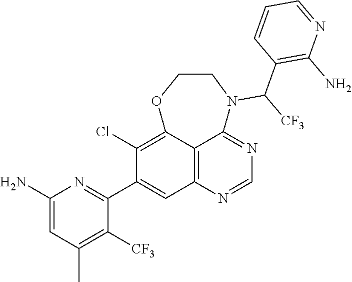 [SMILES: Cc1cc(N)nc(-c2cc3ncnc4c3c(c2Cl)OCCN4C(c2cccnc2N)C(F)(F)F)c1C(F)(F)F]

### Synthetic Route

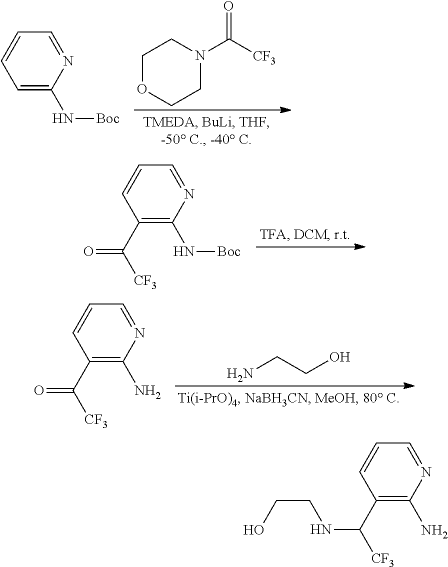

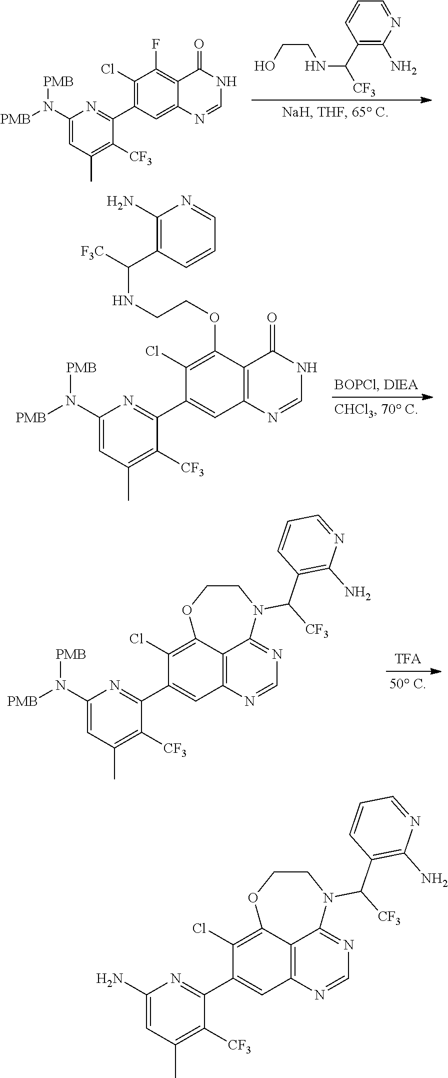

Step 1: tert-butyl (3-(2,2,2-trifluoroacetyl)pyridin-2-yl)carbamate

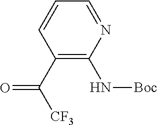 [SMILES: CC(C)(C)OC(=O)Nc1ncccc1C(=O)C(F)(F)F]

Under nitrogen, a solution of tert-butyl pyridin-2-ylcarbamate (1.00 g, 5.15 mmol), N,N,N′,N′-tetramethylethylenediamine (1.62 g, 13.90 mmol) in tetrahydrofuran (10.0 mL) was added n-butyllithium (5.12 mL, 12.87 mmol, 2.5M in hexane) at −50° C. The resulting solution was stirred for 2 h at 0° C. Then 2,2,2-trifluoro-1-morpholinoethan-1-one (1.88 g, 10.30 mmol) was added and stirred at −50° C. for 1 hour. The reaction was quenched with ammonia chloride solution. After completion, the reaction mixture was diluted with ethyl acetate. The resulting solution was washed with water, dried over anhydrous sodium sulfate and concentrated under vacuum. The residue was purified by flash chromatography on silica gel eluting with petroleum ether/ethyl acetate (10/1) to afford tert-butyl (3-(2,2,2-trifluoroacetyl)pyridin-2-yl)carbamate (660.0 mg, 2.27 mmol, 44.2% yield) as a yellow solid. LC-MS: (ESI, m/z): 291.2 [M+H]⁺.

Step 2: 1-(2-aminopyridin-3-yl)-2,2,2-trifluoroethan-1-one

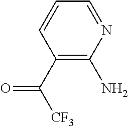 [SMILES: Nc1ncccc1C(=O)C(F)(F)F]

A solution of tert-butyl (3-(2,2,2-trifluoroacetyl)pyridin-2-yl)carbamate (600.0 mg, 2.07 mmol) in dichloromethane (5 mL) and trifluoroacetic acid (1 mL) was stirred at 25° C. for 1 hour. After completion, the reaction mixture was concentrated under vacuum, adjusted to pH 7-8 with sodium carbonate. the reaction mixture was diluted with ethyl acetate. The resulting solution was washed with water, dried over anhydrous sodium sulfate and concentrated under vacuum. The residue was purified by flash chromatography on silica gel eluting with petroleum ether/ethyl acetate (2/1) to afford (2-aminopyridin-3-yl)-2,2,2-trifluoroethan-1-one (390.0 mg, 1.78 mmol, 99.7% yield) as a yellow solid. LC-MS: (ESI, m/z): 191.1 [M+H]⁺.

Step 3: 2-((1-(2-aminopyridin-3-yl)-2,2,2-trifluoroethyl)amino)ethan-1-ol

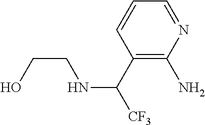 [SMILES: Nc1ncccc1C(NCCO)C(F)(F)F]

A solution of -(2-aminopyridin-3-yl)-2,2,2-trifluoroethan-1-one (2.00 g, 10.52 mmol), 2-aminoethan-1-ol (1.27 g, 21.04 mmol) and tetrapropyl titanate (8.46 g, 31.56 mmol) in methanol (20.00 mL) was stirred at 80° C. for 16 hours. Then sodium cyanoborohydride (1.32 g, 21.04 mmol) was added and stirred at 80° C. for 2 hours. After completion, the reaction was quenched with water. The solvent was concentrated under vacuum. The residue was purified by flash chromatography on silica gel eluting with dichloromethane/methanol (1/10) to afford 2-((1-(2-aminopyridin-3-yl)-2,2,2-trifluoroethyl)amino)ethan-1-ol (1.00 g, 4.25 mmol, 40.4% yield) as a white solid. LC-MS: (ESI, m/z): 235.2 [M+H]⁺.

Step 4: 5-(2-((1-(2-aminopyridin-3-yl)-2,2,2-trifluoroethyl)amino)ethoxy)-7-(6-(bis(4-methoxybenzyl)amino)-4-methyl-3-(trifluoromethyl)pyridin-2-yl)-6-chloroquinazolin-4(3H)-one

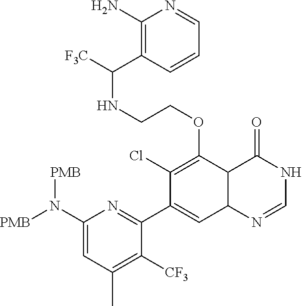

A solution of 2-((1-(2-aminopyridin-3-yl)-2,2,2-trifluoroethyl)amino)ethan-1-ol (383.7 mg, 1.63 mmol) and sodium hydride (97.8 mg, 2.45 mmol, 60% purity) in tetrahydrofuran (5 mL) was stirred at 0° C. for 5 minutes. Then 7-(6-(bis(4-methoxybenzyl)amino)-4-methyl-3-(trifluoromethyl)pyridin-2-yl)-6-chloro-5-fluoroquinazolin-4(3H)-one (500.0 mg, 0.82 mmol) was added and stirred at 65° C. for 1 hour. After completion, the reaction mixture was adjusted to pH 7-8 with hydrochloric acid. The solvent was concentrated under vacuum. The reaction mixture was diluted with ethyl acetate. The resulting solution was washed with water, dried over anhydrous sodium sulfate and concentrated under vacuum. The residue was purified by flash chromatography on silica gel eluting with dichloromethane/methanol (10/1) to afford 5-(2-((1-(2-aminopyridin-3-yl)-2,2,2-trifluoroethyl)amino)ethoxy)-7-(6-(bis(4-methoxybenzyl)amino)-4-methyl-3-(trifluoromethyl)pyridin-2-yl)-6-chloroquinazolin-4(3H)-one (300.0 mg, 0.36 mmol, 44.4% yield) as a yellow solid. LC-MS: (ESI, m/z): 828.2 [M+H]⁺.

Step 5: 6-(4-(1-(2-aminopyridin-3-yl)-2,2,2-trifluoroethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

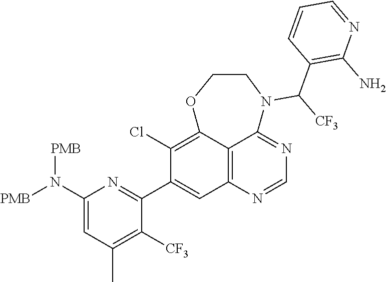

A solution of 5-(2-((1-(2-aminopyridin-3-yl)-2,2,2-trifluoroethyl)amino)ethoxy)-7-(6-(bis(4-methoxybenzyl)amino)-4-methyl-3-(trifluoromethyl)pyridin-2-yl)-6-chloroquinazolin-4(3H)-one (120.0 mg, 0.14 mmol) and N,N-diisopropylethylamine (56.5 mg, 0.43 mmol), bis(2-oxo-3-oxazolidinyl)phosphinic chloride (73.6 mg, 0.29 mmol) in chloroform (1.50 mL) was stirred at 70° C. for 48 hours. After completion, the reaction mixture was diluted with dichloromethane. The resulting solution was washed with water, dried over anhydrous sodium sulfate and concentrated under vacuum. The residue was purified by flash chromatography on silica gel eluting with dichloromethane/methanol (10/1) to afford 6-(4-(1-(2-aminopyridin-3-yl)-2,2,2-trifluoroethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (28.0 mg, 0.03 mmol, 23.9% yield) as a yellow solid. LC-MS: (ESI, m/z): 810.2 [M+H]⁺.

Step 6: 6-(4-(1-(2-aminopyridin-3-yl)-2,2,2-trifluoroethyl)-8-chloro-5,6-dihydro-4H-[1,4]oxazepino[5,6,7-de]quinazolin-9-yl)-4-methyl-5-(trifluoromethyl)pyridin-2-amine

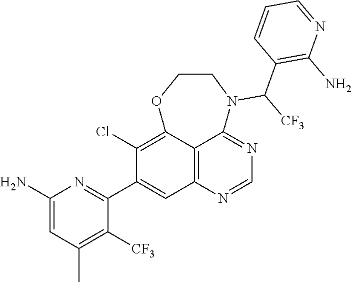 [SMILES: Cc1cc(N)nc(-c2cc3ncnc4c3c(c2Cl)OCCN4C(c2cccnc2N)C(F)(F)F)c1C(F)(F)F]

A solution of 6-(4-(1-(2-aminopyridin-3-yl)-2,2,2-trifluoroethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (28.0 mg, 0.03 mmol) in trifluoroacetic acid (0.5 mL) was stirred at 25° C. for 4 hours. After completion, the solvent was concentrated under vacuum. The residue was purified by flash chromatography on silica gel eluting with dichloromethane/methanol (12/1) to afford 27 mg crude. The crude product was purified by Prep-HPLC with the following conditions: Column: XBridge Prep OBD C18 Column, 19*250 mm, 5 um; Mobile Phase A: Water (10 mmoL/L NH₄HCO₃), Mobile Phase B: ACN; Flow rate: 25 mL/min; Gradient: 48 B to 60 B in 7 min; 254 nm; RT1: 6.57 to afford 6-(4-(1-(2-aminopyridin-3-yl)-2,2,2-trifluoroethyl)-8-chloro-5,6-dihydro-4H-[1,4]oxazepino[5,6,7-de]quinazolin-9-yl)-4-methyl-5-(trifluoromethyl)pyridin-2-amine (4.0 mg, 0.007 mmol, 0.5% yield). LC-MS: (ESI, m/z): 569.9 [M+H]⁺.

Example 20: ¹H NMR (300 MHz, DMSO-d6, ppm) δ 8.65 (d, J=3.6 Hz, 1H), 8.10 (dt, J=5.0, 1.6 Hz, 1H), 7.71 (s, 1H), 7.51-7.28 (m, 2H), 6.84-6.66 (m, 3H), 6.47 (s, 1H), 5.90 (d, J=19.1 Hz, 2H), 4.69-4.33 (m, 2H), 3.87-3.48 (m, 2H), 2.36 (s, 3H).

Example 21: 6-(8-chloro-4-(6,7-dihydro-5H-cyclopenta[c]pyridin-7-yl)-5,6-dihydro-4H-[1,4]oxazepino[5,6,7-de]quinazolin-9-yl)-4-methyl-5-(trifluoromethyl)pyridin-2-amine

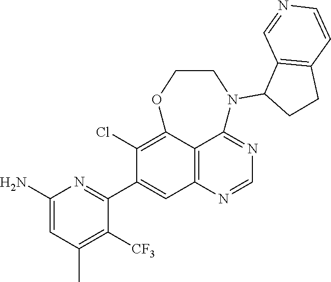 [SMILES: Cc1cc(N)nc(-c2cc3ncnc4c3c(c2Cl)OCCN4C2CCc3ccncc32)c1C(F)(F)F]

### Synthetic Route

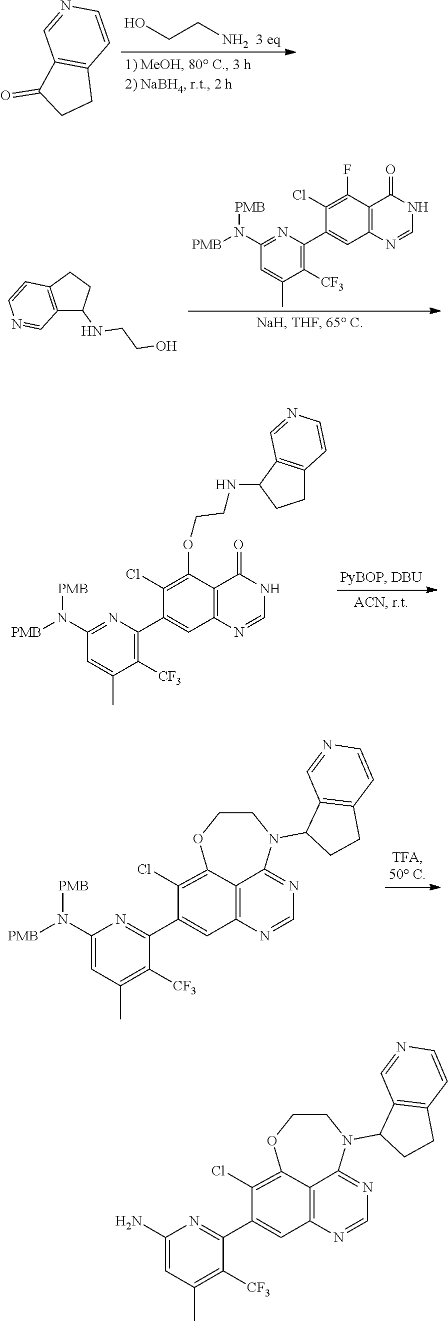

Step 1: 2-((6,7-dihydro-5H-cyclopenta[c]pyridin-7-yl)amino)ethan-1-ol

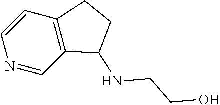 [SMILES: OCCNC1CCc2ccncc21]

A solution of 5,6-dihydrocyclopenta[c]pyridin-7-one (2.0 g, 15.02 mmol), 2-aminoethanol (2.7 mL, 45.06 mmol) and titanium tetraisopropanolate (12.8 g, 45.06 mmol) in methyl alcohol (20 mL) was stirred at 80° C. for 3 hours. Then sodium borohydride (1.1 g, 30.04 mmol) was added and stirred at 25° C. for 2 hours. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (1/1) to afford 2-(6,7-dihydro-5H-cyclopenta[c]pyridin-7-ylamino)ethanol (1.1 g, 6.12 mmol, 40.8% yield) as a black oil. LC-MS: (ESI, m/z): 179.1 [M+H]⁺.

Step 2: 7-(6-(bis(4-methoxybenzyl)amino)-4-methyl-3-(trifluoromethyl)pyridin-2-yl)-6-chloro-5-(2-((6,7-dihydro-5H-cyclopenta[c]pyridin-7-yl)amino)ethoxy)quinazolin-4(3H)-one

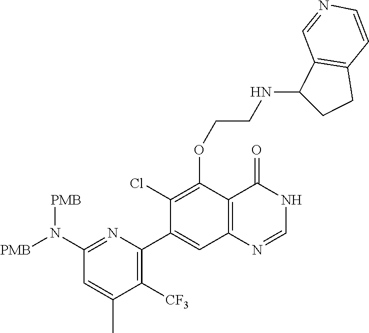

To a solution of 2-(6,7-dihydro-5H-cyclopenta[c]pyridin-7-ylamino)ethanol (232.6 mg, 1.31 mmol) in tetrahydrofuran (2 mL) was added sodium hydride (156.6 mg, 3.92 mmol, 60% purity) and stirred at 0° C. for 0.5 hour. Then 7-[6-[bis[(4-methoxyphenyl)methyl]amino]-4-methyl-3-(trifluoromethyl)-2-pyridyl]-6-chloro-5-fluoro-3H-quinazolin-4-one (400.0 mg, 0.65 mmol) was added and stirred at 65° C. for 0.5 hour. After completion, the reaction mixture was adjusted to pH=7 with hydrochloric acid and concentrated under vacuum. The residue was purified by flash chromatography on silica gel eluting with dichloromethane/methanol (10/1) to afford 7-(6-(bis(4-methoxybenzyl)amino)-4-methyl-3-(trifluoromethyl)pyridin-2-yl)-6-chloro-5-(2-((6,7-dihydro-5H-cyclopenta[c]pyridin-7-yl)amino)ethoxy)quinazolin-4(3H)-one (400.0 mg, 0.50 mmol, 77.9% yield) as a yellow solid. LC-MS: (ESI, m/z): 771.3 [M+H]⁺.

Step 3: 6-(8-chloro-4-(6,7-dihydro-5H-cyclopenta[c]pyridin-7-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

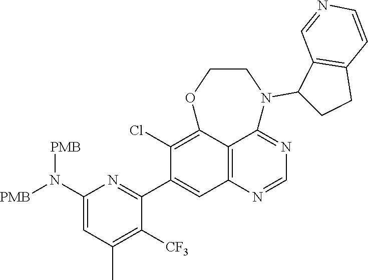

To a solution of 7-(6-(bis(4-methoxybenzyl)amino)-4-methyl-3-(trifluoromethyl)pyridin-2-yl)-6-chloro-5-(2-((6,7-dihydro-5H-cyclopenta[c]pyridin-7-yl)amino)ethoxy)quinazolin-4(3H)-one (350.0 mg, 0.45 mmol) and 1,8-diazabicyclo[5.4.0]undec-7-ene (0.2 mL, 1.82 mmol) in acetonitrile (3 mL) was added benzotriazol-1-yloxytris(dimethylamino)-phosphonium hexafluorophosphate (307.0 mg, 0.59 mmol), the mixture was stirred at room temperature for 1 hour. After completion, the reaction mixture was concentrated under vacuum and purified by reverse phase chromatography (acetonitrile 0-40/0.1% NH₄HCOs in water) to afford 6-(8-chloro-4-(6,7-dihydro-5H-cyclopenta[c]pyridin-7-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (280.0 mg, 0.31 mmol, 70.4% yield) as a yellow solid. LC-MS: (ESI, m/z): 753.2 [M+H]⁺.

Step 4: 6-(8-chloro-4-(6,7-dihydro-5H-cyclopenta[c]pyridin-7-yl)-5,6-dihydro-4H-[1,4]oxazepino[5,6,7-de]quinazolin-9-yl)-4-methyl-5-(trifluoromethyl)pyridin-2-amine

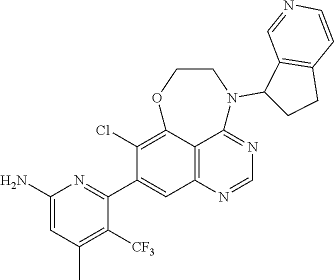 [SMILES: Cc1cc(N)nc(-c2cc3ncnc4c3c(c2Cl)OCCN4C2CCc3ccncc32)c1C(F)(F)F]

A solution of 6-(8-chloro-4-(6,7-dihydro-5H-cyclopenta[c]pyridin-7-yl)-5,6-dihydro-4H-[1,4]oxazepino[5,6,7-de]quinazolin-9-yl)-4-methyl-5-(trifluoromethyl)pyridin-2-amine (150.0 mg, 0.20 mmol) in 2,2,2-trifluoroacetic acid (4 mL) was stirred at 50° C. for 8 hours. After completion, the mixture was concentrated under vacuum and purified by Prep-HPLC with the following conditions: (Column: YMC-Actus Triart C18 ExRS, 30*250 mm, 5 μm; Mobile Phase A: water (10 mmol/L NH₄HCO₃), Mobile Phase B: acetonitrile; Flow rate: 60 mL/min; Gradient: 15% B to 33% B in 10 min; 254/220 nm; RT: 10.38 min) to afford 6-(8-chloro-4-(6,7-dihydro-5H-cyclopenta[c]pyridin-7-yl)-5,6-dihydro-4H-[1,4]oxazepino[5,6,7-de]quinazolin-9-yl)-4-methyl-5-(trifluoromethyl)pyridin-2-amine (9.5 mg, 0.017 mmol, 8.9% yield). LC-MS: (ESI, m/z): 513.2 [M+H]⁺.

Example 21: ¹H NMR (300 MHz, DMSO-d₆, ppm) δ 8.50-8.42 (m, 3H), 7.37 (d, J=5.0 Hz, 1H), 7.21 (s, 1H), 6.90-6.83 (m, 1H), 6.74 (s, 2H), 6.44 (s, 1H), 4.63-4.41 (m, 2H), 3.68-3.45 (m, 2H), 3.07-2.92 (m, 2H), 2.57-2.47 (m, 1H), 2.35 (s, 3H), 2.13-1.03 (m, 1H).

Example 22: 6-(8-chloro-4-(isothiazol-5-ylmethyl)-5,6-dihydro-4H-[1,4]oxazepino[5,6,7-de]quinazolin-9-yl)-4-methyl-5-(trifluoromethyl)pyridin-2-amine

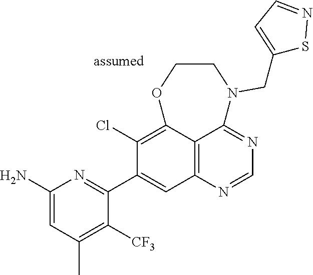 [SMILES: Cc1cc(N)nc(-c2cc3ncnc4c3c(c2Cl)OCCN4Cc2ccns2)c1C(F)(F)F]

### Synthetic Route

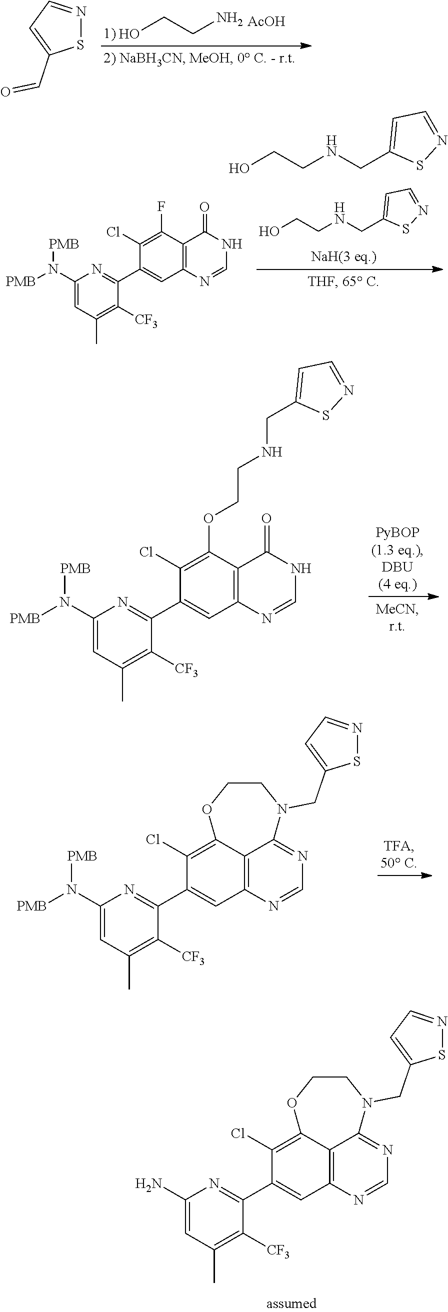

Step 1: 2-((isothiazol-5-ylmethyl)amino)ethan-1-ol

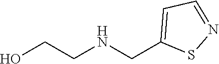

A solution of isothiazole-5-carbaldehyde (2.00 g, 17.68 mmol), 2-aminoethanol (2 mL, 35.35 mmol) and acetic acid (0.1 mL, 1.77 mmol) in methyl alcohol (10 mL) was stirred at 25° C. for 0.5 hour. Then sodium cyanoborohydride (2.22 g, 35.35 mmol) was added and stirred at 25° C. for 1 hour. After completion, the reaction was quenched with water (0.5 mL) and concentrated under vacuum. The residue was purified by flash chromatography on silica gel eluting with dichloromethane/methanol (10/1) to afford 2-((isothiazol-5-ylmethyl)amino)ethan-1-ol (870.3 mg, 5.10 mmol, 28.9% yield) as a yellow solid. LC-MS: (ESI, m/z): 159.1 [M+H]⁺.

Step 2: 7-(6-(bis(4-methoxybenzyl)amino)-4-methyl-3-(trifluoromethyl)pyridin-2-yl)-6-chloro-5-(2-((isothiazol-5-ylmethyl)amino)ethoxy)quinazolin-4(3H)-one

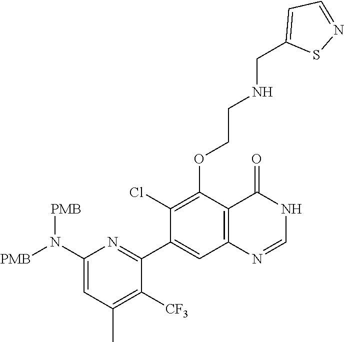

To a solution of 2-((isothiazol-5-ylmethyl)amino)ethan-1-ol (206.4 mg, 1.31 mmol) in tetrahydrofuran (4 mL) was added sodium hydride (156.6 mg, 3.92 mmol, 60% purity) and stirred at 25° C. for 0.5 hour. Then 7-(6-(bis(4-methoxybenzyl)amino)-4-methyl-3-(trifluoromethyl)pyridin-2-yl)-6-chloro-5-fluoroquinazolin-4(3H)-one (400.0 mg, 0.65 mmol) was added and stirred at 65° C. for 2 hours. The reaction was quenched with saturated ammonium chloride solution. The resulting solution diluted with water and extracted with ethyl acetate. The organic layers were washed with brine, dried over anhydrous sodium sulfate and concentrated. The residue was purified by flash chromatography on silica gel eluting with dichloromethane/methanol (10/1) to afford 7-(6-(bis(4-methoxybenzyl)amino)-4-methyl-3-(trifluoromethyl)pyridin-2-yl)-6-chloro-5-(2-((isothiazol-5-ylmethyl)amino)ethoxy)quinazolin-4(3H)-one (690.0 mg, 0.63 mmol, 96.8% yield) as a yellow solid. LC-MS: (ESI, m/z): 751.2 [M+H]⁺.

Step 3: 6-(8-chloro-4-(isothiazol-5-ylmethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

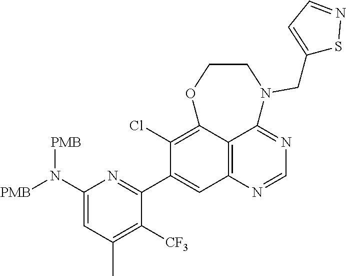

To a solution of 7-(6-(bis(4-methoxybenzyl)amino)-4-methyl-3-(trifluoromethyl)pyridin-2-yl)-6-chloro-5-(2-((isothiazol-5-ylmethyl)amino)ethoxy)quinazolin-4(3H)-one (660.0 mg, 0.88 mmol) and 1,8-diazabicyclo[5.4.0]undec-7-ene (0.5 mL, 3.51 mmol) in acetonitrile (6 mL) was added benzotriazol-1-yloxytris(dimethylamino)-phosphonium hexafluorophosphate (594.3 mg, 1.14 mmol) and stirred at 25° C. for 3 hours. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (10/1) to afford 6-(8-chloro-4-(isothiazol-5-ylmethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (229.0 mg, 0.29 mmol, 33.7% yield) as a yellow solid. LC-MS: (ESI, m/z): 733.2 [M+H]⁺.

Step 4: 6-(8-chloro-4-(isothiazol-5-ylmethyl)-5,6-dihydro-4H-[1,4]oxazepino[5,6,7-de]quinazolin-9-yl)-4-methyl-5-(trifluoromethyl)pyridin-2-amine

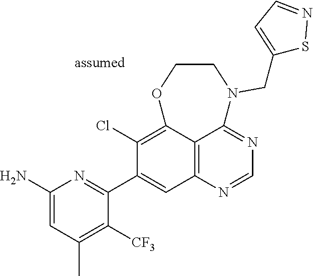 [SMILES: Cc1cc(N)nc(-c2cc3ncnc4c3c(c2Cl)OCCN4Cc2ccns2)c1C(F)(F)F]

A solution of 6-(8-chloro-4-(isothiazol-5-ylmethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (279.0 mg, 0.38 mmol) in 2,2,2-trifluoroacetic acid (0.5 mL) was stirred at 50° C. for 8 hours. The solvent was removed under vacuum. The crude product was purified by Prep-HPLC with the following conditions: (Column: XBridge Prep OBD C18 Column, 19×250 mm, 5 um; Mobile Phase A: water (10 mmol/L NH₄HCOs), Mobile Phase B: methanol; Flow rate: 25 mL/min; Gradient: 60% B to 85% B in 7 min; 254 nm; RT: 5.6 min) to afford 6-(8-chloro-4-(isothiazol-5-ylmethyl)-5,6-dihydro-4H-[1,4]oxazepino[5,6,7-de]quinazolin-9-yl)-4-methyl-5-(trifluoromethyl)pyridin-2-amine (23.7 mg, 0.04 mmol, 12.6% yield). LC-MS: (ESI, m/z): 493.1 [M+H]⁺.

Example 22: ¹H NMR (300 MHz, DMSO-d₆, ppm) δ 8.60 (s, 1H), 8.48 (d, J=1.6 Hz, 1H), 7.53 (d, J=1.7 Hz, 1H), 7.24 (s, 1H), 6.78 (s, 2H), 6.46 (s, 1H), 5.21 (s, 2H), 4.67-4.53 (m, 2H), 4.13-3.94 (m, 2H), 2.36 (d, J=1.5 Hz, 3H).

Example 23: 4-(8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)benzo[d]thiazol-2-amine

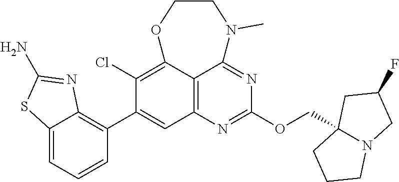 [SMILES: CN1CCOc2c(Cl)c(-c3cccc4sc(N)nc34)cc3nc(OC[C@@]45CCCN4C[C@H](F)C5)nc1c23]

### Synthetic Route

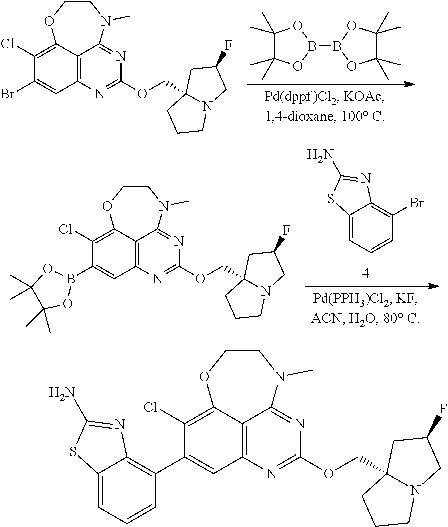

Step 1: 8-chloro-2-(((2R,7aS)-2-fluorotetrahydro-1H-pyrrolizin-7a(5H)-yl)methoxy)-4-methyl-9-(4,4,5,5-tetramethyl-1,3,2-dioxaborolan-2-yl)-5,6-dihydro-4H-[1,4]oxazepino[5,6,7-de]quinazoline

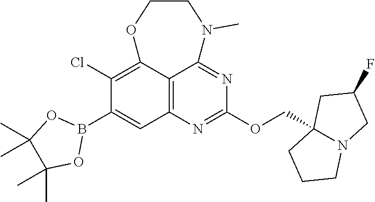 [SMILES: CN1CCOc2c(Cl)c(B3OC(C)(C)C(C)(C)O3)cc3nc(OC[C@@]45CCCN4C[C@H](F)C5)nc1c23]

Under nitrogen, a solution of 9-bromo-8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazoline (986.0 mg, 2.09 mmol), 4,4,4′,4′,5,5,5′,5′-octamethyl-2,2′-bi(1,3,2-dioxaborolane) (2653.7 mg, 10.40 mmol), potassium acetate (615.3 mg, 6.27 mmol) and [1,1′-bis(diphenylphosphino)ferrocene]dichloropalladium(II) (155.0 mg, 0.21 mmol) in 1,4-dioxane (6 mL) was stirred at 100° C. for 2 hours. After completion, the reaction mixture was concentrated under vacuum. The mixture was filtered and washed with dichloromethane. The filtrate was concentrated under reduced pressure. The crude (1.2 g, crude) was used directly without further purification. LC-MS: (ESI, m/z): 519.2 [M+H]⁺.

Step 2: 4-(8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)benzo[d]thiazol-2-amine

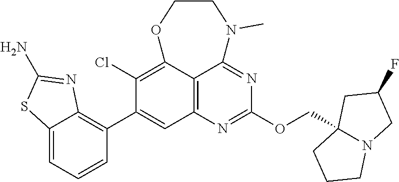 [SMILES: CN1CCOc2c(Cl)c(-c3cccc4sc(N)nc34)cc3nc(OC[C@@]45CCCN4C[C@H](F)C5)nc1c23]

Under nitrogen, a solution of 8-chloro-2-(((2R,7aS)-2-fluorotetrahydro-1H-pyrrolizin-7a(5H)-yl)methoxy)-4-methyl-9-(4,4,5,5-tetramethyl-1,3,2-dioxaborolan-2-yl)-5,6-dihydro-4H-[1,4]oxazepino[5,6,7-de]quinazoline (900.0 mg, crude), 4-bromo-1,3-benzothiazol-2-amine (139.1 mg, 0.61 mmol), potassium fluoride (70.5 mg, 1.21 mmol) and bis(triphenylphosphine)palladium(II) chloride (42.7 mg, 0.06 mmol) in acetonitrile (5 mL) and water (1 mL) was stirred at 80° C. for 2 hours. After completion, the reaction mixture was diluted with dichloromethane and washed with water. The organic layer was dried over anhydrous sodium sulfate and concentrated under vacuum. The residue was purified by preparative TLC with dichloromethane/methanol (20/1) to afford crude product. The crude product was purified by Prep-HPLC with the following conditions: (Column: XBridge Prep OBD C18 Column, 30×150 mm 5 um; Mobile Phase A: water (10 mmol/L NH₄HCO₃), Mobile Phase B: acetonitrile; Flow rate: 60 mL/min; Gradient: 32% B to 62% B in 7 min; 254 nm; RT1: 6.5 min) to afford 4-(8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)benzo[d]thiazol-2-amine (48.1 mg, 0.085 mmol, 14.1% yield). LC-MS: (ESI, m/z): 541.2 [M+H]⁺.

Example 23: ¹H NMR (300 MHz, DMSO-d₆, ppm) δ 7.70 (dd, J=6.7, 2.5 Hz, 1H), 7.59 (s, 2H), 7.13-7.06 (m, 3H), 5.26 (d, J=55.1 Hz, 1H), 4.57 (dd, J=5.8, 2.8 Hz, 2H), 4.05 (d, J=10.4 Hz, 1H), 3.93 (dd, J=9.7, 7.1 Hz, 3H), 3.29 (s, 3H), 3.11-3.08 (m, 2H), 3.00 (s, 1H), 2.86-2.78 (m, 1H), 2.14-1.99 (m, 3H), 1.84-1.76 (m, 3H).

Example 24: 6-(8-chloro-4-methyl-5,6-dihydro-4H-[1,4]oxazepino[5,6,7-de]quinazolin-9-yl)-4-methyl-5-(trifluoromethyl)pyridin-2-amine

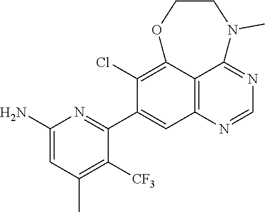 [SMILES: Cc1cc(N)nc(-c2cc3ncnc4c3c(c2Cl)OCCN4C)c1C(F)(F)F]

### Synthetic Route

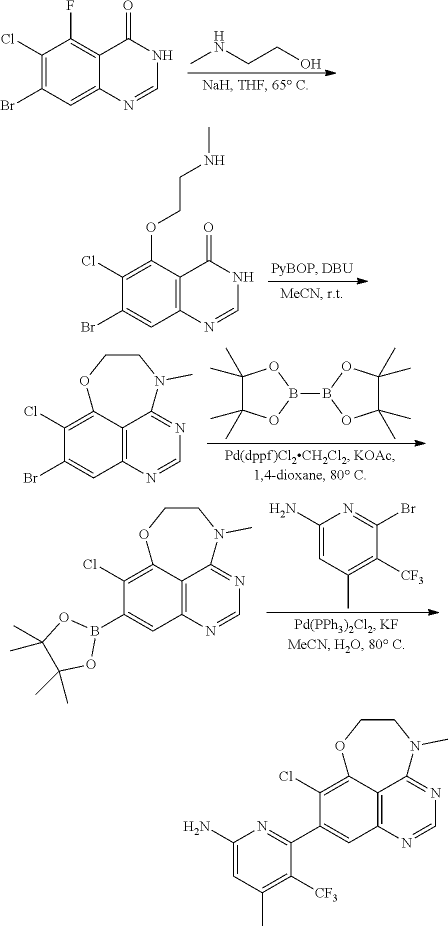

Step 1:7-bromo-6-chloro-5-(2-(methylamino)ethoxy)quinazolin-4(3H)-one

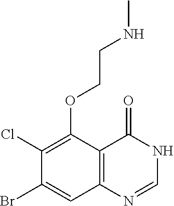 [SMILES: CNCCOc1c(Cl)c(Br)cc2nc[nH]c(=O)c12]

A solution of 2-(methylamino)ethanol (1.38 g, 18.38 mmol) and sodium hydride (735.1 mg, 18.3 mmol, 60% purity) in tetrahydrofuran (10 mL) was stirred at 25° C. for 0.5 hour. Then 7-bromo-6-chloro-5-fluoro-3H-quinazolin-4-one (1.7 g, 6.13 mmol) was added and stirred at 65° C. for 1 hour. After completion, the reaction was quenched with 1 M hydrochloric acid (5 mL). The solvent was concentrated under vacuum. The residue was purified by flash chromatography on silica gel eluting with methanol/dichloromethane (1/10) to afford 7-bromo-6-chloro-5-(2-(methylamino)ethoxy)quinazolin-4(3H)-one (2.10 g, 5.49 mmol, 89.7% yield) as a yellow solid. LC-MS: (ESI, m/z): 332.1 [M+H]⁺

Step 2: 9-bromo-8-chloro-4-methyl-5,6-dihydro-4H-[1,4]oxazepino[5,6,7-de]quinazoline

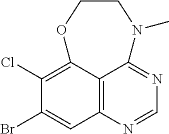 [SMILES: CN1CCOc2c(Cl)c(Br)cc3ncnc1c23]

To a solution of 7-bromo-6-chloro-5-(2-(methylamino)ethoxy)quinazolin-4(3H)-one (2.10 g, 6.31 mmol) and benzotriazol-1-yloxytris(dimethylamino)-phosphonium hexafluorophosphate (4.93 g, 9.47 mmol) in acetonitrile (10 mL) was added 1,8-diazabicyclo[5.4.0]undec-7-ene (2.88 g, 18.94 mmol), the mixture was stirred at 25° C. for 1 hour. After completion, the solvent was concentrated under vacuum. The resulting residue was purified by reverse phase chromatography (acetonitrile 50-54/0.1% FA in water) to afford 9-bromo-8-chloro-4-methyl-5,6-dihydro-4H-[1,4]oxazepino[5,6,7-de]quinazoline (1.50 g, 4.61 mmol, 73.1% yield) as a yellow solid. LC-MS: (ESI, m/z): 314.0 [M+H]⁺

Step 3: 8-chloro-4-methyl-9-(4,4,5,5-tetramethyl-1,3,2-dioxaborolan-2-yl)-5,6-dihydro-4H-[1,4]oxazepino[5,6,7-de]quinazoline

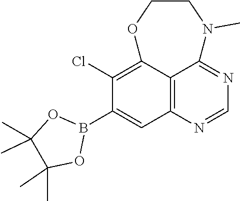

Under nitrogen, a mixture of 9-bromo-8-chloro-4-methyl-5,6-dihydro-4H-[1,4]oxazepino[5,6,7-de]quinazoline (800.0 mg, 2.5 mmol), bis(pinacolato)diboron (1.29 g, 5.09 mmol), [1,1′-bis(diphenylphosphino)ferrocene]dichloropalladium(II) (188.6 mg, 0.2 mmol) and potassium acetate (498.4 mg, 5.0 mmol) in 1,4-dioxane (8 mL) was stirred at 100° C. for 2 hours. After completion, the reaction mixture was filtered and washed with ethyl acetate. The filtrate was concentrated under reduced pressure. The crude product (1.2 g, crude) was used directly without further purification. LC-MS: (ESI, m/z): 362.1 [M+H]⁺

Step 4:6-(8-chloro-4-methyl-5,6-dihydro-4H-[1,4]oxazepino[5,6,7-de]quinazolin-9-yl)-4-methyl-5-(trifluoromethyl)pyridin-2-amine

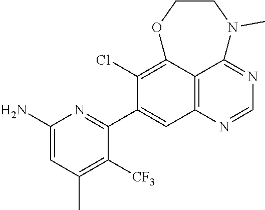 [SMILES: Cc1cc(N)nc(-c2cc3ncnc4c3c(c2Cl)OCCN4C)c1C(F)(F)F]

Under nitrogen, a mixture of 8-chloro-4-methyl-9-(4,4,5,5-tetramethyl-1,3,2-dioxaborolan-2-yl)-5,6-dihydro-4H-[1,4]oxazepino[5,6,7-de]quinazoline (2.20 g, crude), 6-bromo-4-methyl-5-(trifluoromethyl)pyridin-2-amine (620.6 mg, 2.4 mmol), bis(triphenylphosphine)palladium(II) chloride (427.0 mg, 0.6 mmol) and potassium fluoride (705.6 mg, 12.1 mmol) in acetonitrile (10 mL) and water (2 mL) was stirred at 80° C. for 1 hour. The resulting solution was diluted with water and extracted with ethyl acetate. The organic layers were washed with brine, dried over anhydrous sodium sulfate and concentrated. The resulting residue was purified by reverse phase chromatography (acetonitrile 40-45/0.1% TFA in water) to afford crude product. The crude product was purified by Prep-HPLC with the following conditions: (Column: XBridge Prep OBD C18 Column, 30×150 mm 5 um; Mobile Phase A: Water (10 mmol/L NH₄HCO₃), Mobile Phase B: ACN; Flow rate: 60 mL/min; Gradient: 33 B to 63 B in 7 min; 254 nm; RT: 6.33 min) to afford 6-(8-chloro-4-methyl-5,6-dihydro-4H-[1,4]oxazepino[5,6,7-de]quinazolin-9-yl)-4-methyl-5-(trifluoromethyl)pyridin-2-amine (77.6 mg, 0.1 mmol, 3.1% yield). LC-MS: (ESI, m/z): 410.1 [M+H]⁺

Example 24: ¹H NMR (300 MHz, DMSO-d₆, ppm) δ 8.43 (s, 1H), 7.16 (s, 1H), 6.78 (s, 2H), 6.48-6.42 (m, 1H), 4.72-4.52 (m, 2H), 4.06-3.85 (m, 2H), 3.31 (s, 3H), 2.40-2.32 (m, 3H).

Example 25: 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)thiazol-4-amine

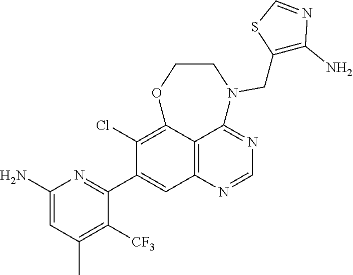 [SMILES: Cc1cc(N)nc(-c2cc3ncnc4c3c(c2Cl)OCCN4Cc2scnc2N)c1C(F)(F)F]

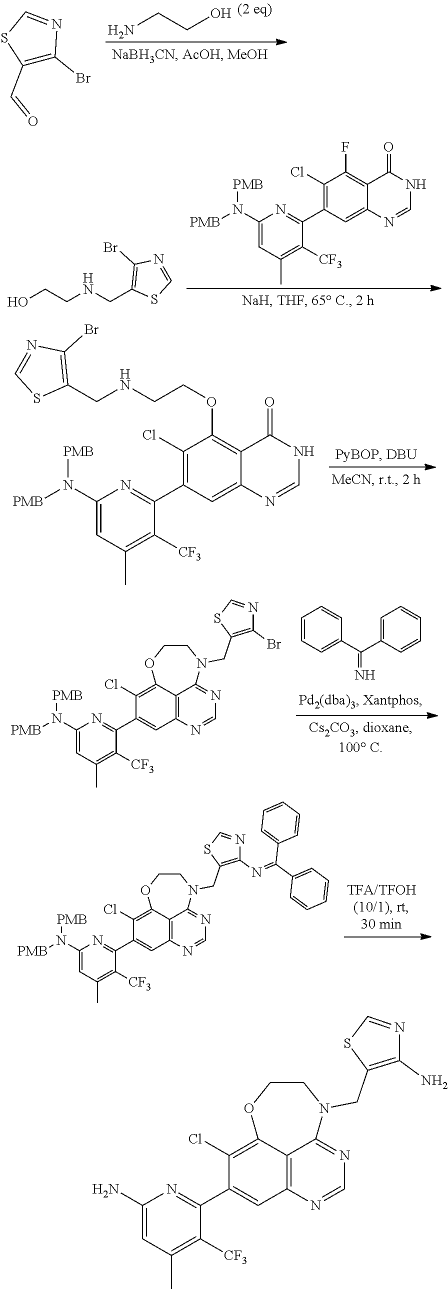

Step 1: 2-(((4-bromothiazol-5-yl)methyl)amino)ethan-1-ol

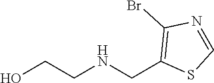 [SMILES: OCCNCc1scnc1Br]

To a mixture of 2-aminoethanol (200 mg, 3.27 mmol) in methyl alcohol (15 mL) was added acetic acid (2.00 g, 33.31 mmol) and 4-bromo-1,3-thiazole-5-carbaldehyde (500 mg, 2.60 mmol), the mixture was stirred for 1 h at 60° C. Then sodium cyanoborohydride (325 mg, 5.17 mmol) was added at room temperature and stirred for 1 h. The solvent was removed under vacuum. The residue was purified by flash chromatography on silica gel eluting with dichloromethane/methanol (93:7) to afford 2-(((4-bromothiazol-5-yl)methyl)amino)ethan-1-ol (550 mg, 2.31 mmol, 89.1% yield) as a yellow solid. LCMS (ESI, m/z): 236.8 [M+H]⁺

Step 2: 7-(6-(bis(4-methoxybenzyl)amino)-4-methyl-3-(trifluoromethyl)pyridin-2-yl)-5-(2-(((4-bromothiazol-5-yl)methyl)amino)ethoxy)-6-chloroquinazolin-4(3H)-one

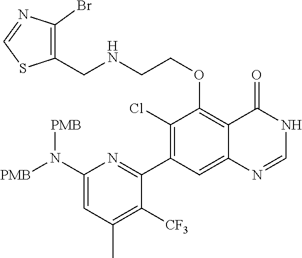

A mixture of 2-(((4-bromothiazol-5-yl)methyl)amino)ethan-1-ol (240 mg, 1.01 mmol), 7-[6-[bis[(4-methoxyphenyl)methyl]amino]-4-methyl-3-(trifluoromethyl)-2-pyridyl]-6-chloro-5-fluoro-3H-quinazolin-4-one (400 mg, 0.65 mmol) and sodium hydride (80 mg, 2.00 mmol, 60% purity) in tetrahydrofuran (10 mL) was stirred at 65° C. for 4 h. The resulting solution was quenched with water, extracted with ethyl acetate, washed with brine, dried over anhydrous sodium sulfate and concentrated. The residue was purified by flash chromatography on silica gel eluting with dichloromethane/methanol (10/1) to afford 7-(6-(bis(4-methoxybenzyl)amino)-4-methyl-3-(trifluoromethyl)pyridin-2-yl)-5-(2-(((4-bromothiazol-5-yl)methyl)amino)ethoxy)-6-chloroquinazolin-4(3H)-one (460 mg, 0.55 mmol, 84.9% yield) as a yellow solid. LCMS (ESI, m/z): 831.1 [M+H]⁺.

Step 3: 6-(4-((4-bromothiazol-5-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

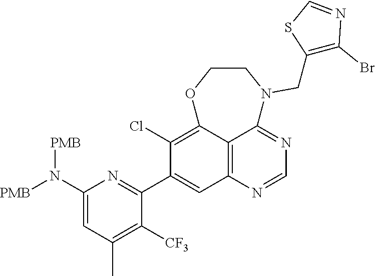

To a mixture of 7-(6-(bis(4-methoxybenzyl)amino)-4-methyl-3-(trifluoromethyl)pyridin-2-yl)-5-(2-(((4-bromothiazol-5-yl)methyl)amino)ethoxy)-6-chloroquinazolin-4(3H)-one (460 mg, 0.55 mmol) in acetonitrile (8 mL) was added 1,8-diazabicyclo[5.4.0]undec-7-ene (258 mg, 1.69 mmol) and benzotriazol-1-yloxytris(dimethylamino)-phosphonium hexafluorophosphate (450 mg, 0.86 mmol), the mixture was stirred for 1 h at room temperature. The resulting solution was diluted with water, extracted with ethyl acetate, washed with brine, dried over anhydrous sodium sulfate and concentrated. The residue was purified by flash chromatography on silica gel eluting with petroleum ether/ethyl acetate (1/1) to afford 6-(4-((4-bromothiazol-5-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.0 mg, 0.49 mmol, 88.9% yield) as a yellow solid. LCMS (ESI, m/z): 813.1 [M+H]⁺

Step 4: 6-(8-chloro-4-((4-((diphenylmethylene)amino)thiazol-5-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

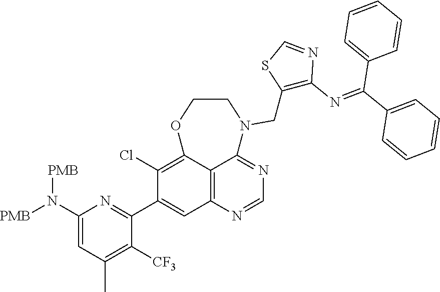

A mixture of 6-(4-((4-bromothiazol-5-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400 mg, 0.49 mmol), benzophenone imine (160 mg, 0.88 mmol), tris(dibenzylideneacetone)dipalladium (50 mg, 0.05 mmol), 1.1′-Binaphthyl-2.2′-diphenyl phosphine (62 mg, 0.10 mmol) and sodium tert-butoxide (100 mg, 1.04 mmol) in toluene (8 mL) was stirred at 100° C. for 2 h under nitrogen. The resulting solution was diluted with water, extracted with ethyl acetate, washed with brine, dried over anhydrous sodium sulfate and concentrated. The residue was purified by flash chromatography on silica gel eluting with petroleum ether/ethyl acetate (1/1) to afford 6-(8-chloro-4-((4-((diphenylmethylene)amino)thiazol-5-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (260.0 mg, 0.28 mmol, 57.9% yield) as a brown solid. LCMS (ESI, m/z): 912.3 [M+H]⁺

Step 5: 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)thiazol-4-amine

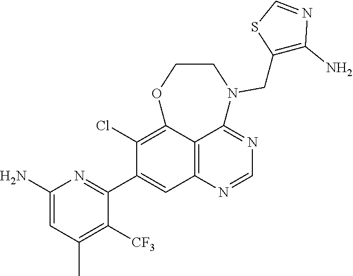 [SMILES: Cc1cc(N)nc(-c2cc3ncnc4c3c(c2Cl)OCCN4Cc2scnc2N)c1C(F)(F)F]

A mixture of 6-(8-chloro-4-((4-((diphenylmethylene)amino)thiazol-5-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200 mg, 0.22 mmol) in trifluoroacetic acid (5 mL) and trifluoromethanesulfonic acid (0.5 mL) was stirred at room temperature for 0.5h. The solvent was removed under vacuum. The reaction mixture was adjusted to pH 8 with saturated sodium bicarbonate solution. The resulting solution was extracted with ethyl acetate and concentrated. The resulting residue was purified by reverse phase chromatography (acetonitrile 0-40/0.1% NH₄HCO₃ in water) to afford the crude product. The crude product was purified by Prep-HPLC with the following conditions: Column: XBridge Prep OBD C18 Column, 30×150 mm Sum: Mobile Phase A: Water (10 mmol/L NH₄HCO₃), Mobile Phase B: ACN; Flow rate: 60 mL/min; Gradient: 31% B to 44% B in 8 min, 44% B; Wave Length: 220/254 nm; RT: 7.87 min. This resulted in 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)thiazol-4-amine (22.7 mg, 0.04 mmol, 20.4% yield). LCMS (ESI, m/z): 507.9 [M+H]⁺.

Example 25: ¹H NMR (300 MHz, DMSO-d₆, ppm) δ 8.64 (s, 1H), 8.52 (s, 1H), 7.19 (s, 1H), 6.78 (s, 2H), 6.45 (s, 1H), 5.65 (s, 2H), 5.04 (d, J=2.1 Hz, 2H), 4.61-4.59 (m, 1H), 4.56-4.55 (m, 1H), 3.95-3.91 (m, 2H), 2.36-2.35 (m, 3H).

Example 26: 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyrimidin-4-amine

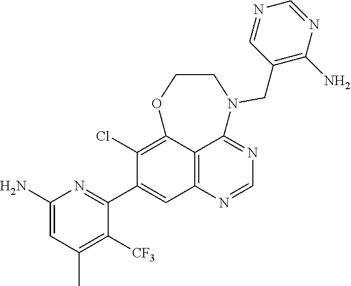 [SMILES: Cc1cc(N)nc(-c2cc3ncnc4c3c(c2Cl)OCCN4Cc2cncnc2N)c1C(F)(F)F]

### Synthetic Route

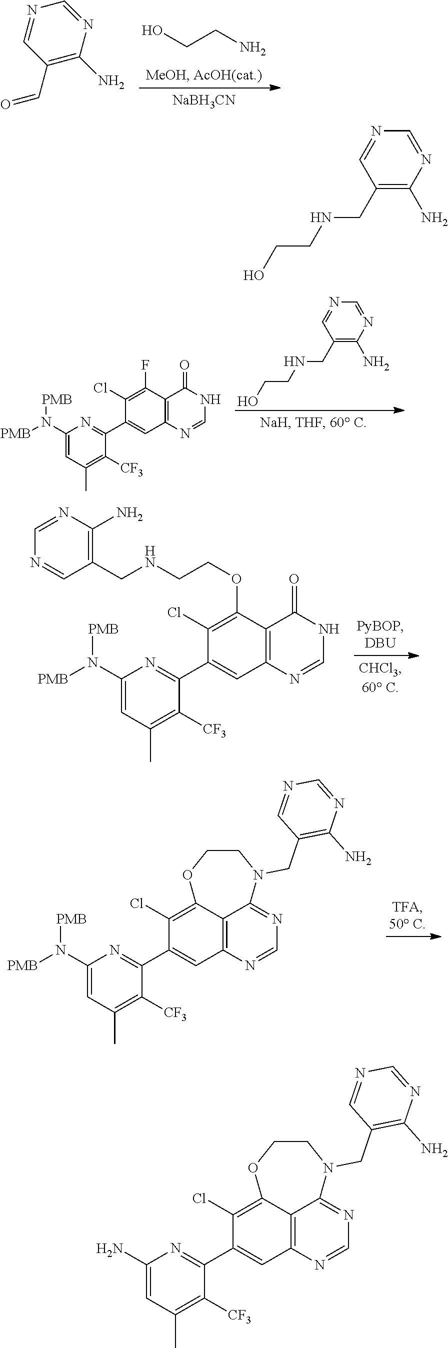

Step 1:2-(((4-aminopyrimidin-5-yl)methyl)amino)ethan-1-ol

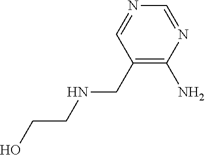 [SMILES: Nc1ncncc1CNCCO]

A solution of 4-aminopyrimidine-5-carbaldehyde (1.0 g, 8.12 mmol), 2-aminoethanol (1.0 g, 16.37 mmol) and acetic acid (100.0 mg, 1.67 mmol) in methyl alcohol (10 mL) was stirred at 25° C. for 1 hour. Then sodium cyanoborohydride (1.02 g, 16.23 mmol) was added and stirred at 25° C. for 1 hour. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (9/1) to afford 2-(((4-aminopyrimidin-5-yl)methyl)amino)ethan-1-ol (350.0 mg, 2.08 mmol, 25.6% yield) as a yellow oil. LC-MS: (ESI, m/z): 169.0 [M+H]⁺

Step 2: 5-(2-(((4-aminopyrimidin-5-yl)methyl)amino)ethoxy)-7-(6-(bis(4-methoxybenzyl)amino)-4-methyl-3-(trifluoromethyl)pyridin-2-yl)-6-chloroquinazolin-4(3H)-one

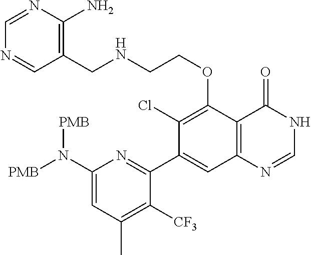

A solution of and 2-(((4-aminopyrimidin-5-yl)methyl)amino)ethan-1-ol (164.0 mg, 0.98 mmol) and sodium hydride (58.0 mg, 1.45 mmol, 60% purity) in tetrahydrofuran (5 mL) was stirred at 0° C. Then 7-(6-(bis(4-methoxybenzyl)amino)-4-methyl-3-(trifluoromethyl)pyridin-2-yl)-6-chloro-5-fluoroquinazolin-4(3H)-one (300.0 mg, 0.49 mmol) was added at 0° C. and stirred at 65° C. for 3 hours. After completion, the reaction was quenched with water and the solvent was concentrated under vacuum. The residue was purified by flash chromatography on silica gel eluting with dichloromethane/methanol (9/1) to afford 5-(2-(((4-aminopyrimidin-5-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (220 mg, 0.29 mmol, 59.1% yield) as a yellow oil. LC-MS: (ESI, m/z): 761.2 [M+H]⁺

Step 3: 5-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyrimidin-4-amine

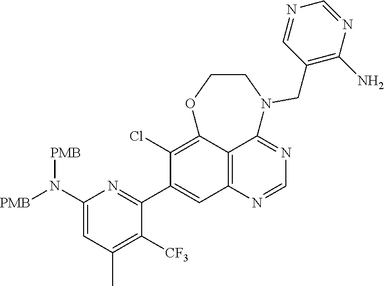

A solution of 5-(2-(((4-aminopyrimidin-5-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (130.0 mg, 0.17 mmol), benzotriazole-1-yl-oxytripyrrolidinophosphonium hexafluorophosphate (104.0 mg, 0.20 mmol) and 1,8-diazabicyclo[5.4.0]undecane-7-ene (78.0 mg, 0.51 mmol) in chloroform (3 mL) was stirred at 60° C. for 1 hour. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (9/1) to afford 5-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyrimidin-4-amine (80.0 mg, 0.11 mmol, 63% yield) as a yellow oil. LC-MS: (ESI, m/z): 743.2 [M+H]⁺

Step 4: 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyrimidin-4-amine

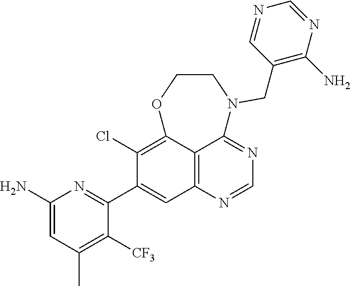 [SMILES: Cc1cc(N)nc(-c2cc3ncnc4c3c(c2Cl)OCCN4Cc2cncnc2N)c1C(F)(F)F]

A solution of 5-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyrimidin-4-amine (90.0 mg, 0.12 mmol) in trifluoroacetic acid (1 mL) was stirred at 60° C. for 5 hours. After completion, the solvent was concentrated under vacuum. The crude product was purified by Prep-HPLC with the following conditions: (Column: YMC-Actus Triart C18 ExRS, 30*150 mm, 5 μm; Mobile Phase A: Water (10 mmol/L NH₄HCO₃), Mobile Phase B: ACN; Flow rate: 60 mL/min; Gradient: 26% B to 42% B in 10 min, 42% B; Wave Length: 220/254 nm; RT: 9.35 min) to afford 5-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyrimidin-4-amine (29.1 mg, 0.05 mmol, 47.8% yield). LC-MS: (ESI, m/z): 503.1 [M+H]⁺

Example 26: ¹H NMR (400 MHz, DMSO-d₆, ppm) δ 8.44 (s, 1H), 8.34 (s, 1H), 8.02 (s, 1H), 7.20 (s, 1H), 7.09 (s, 2H), 6.77 (s, 2H), 6.45 (s, 1H), 4.89-4.76 (m, 2H), 4.70-4.56 (m, 2H), 3.97-3.82 (m, 2H), 2.36 (s, 3H).

Example 27: 6-(4-(2-(2-aminopyridin-3-yl)propan-2-yl)-8-chloro-5,6-dihydro-4H-[1,4]oxazepino[5,6,7-de]quinazolin-9-yl)-4-methyl-5-(trifluoromethyl)pyridin-2-amine

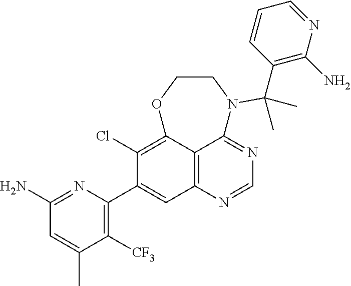 [SMILES: Cc1cc(N)nc(-c2cc3ncnc4c3c(c2Cl)OCCN4C(C)(C)c2cccnc2N)c1C(F)(F)F]

### Synthetic Route

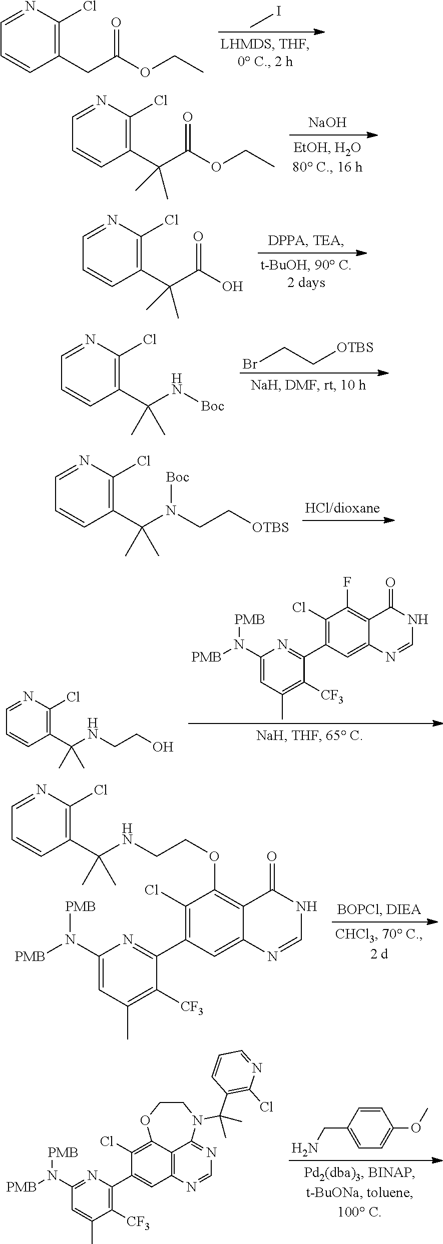

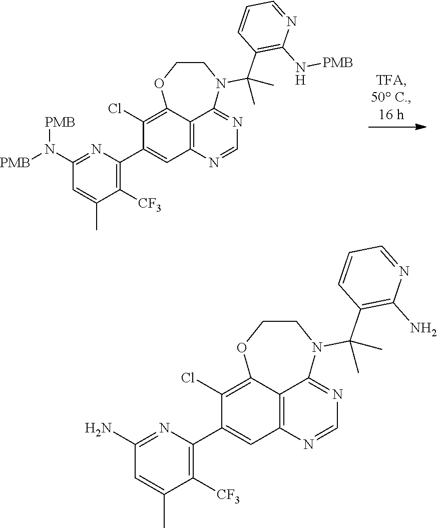

Step 1: ethyl 2-(2-chloropyridin-3-yl)-2-methylpropanoate

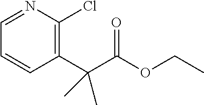

To a mixture of ethyl 2-(2-chloro-3-pyridyl)acetate (1.00 g, 5.01 mmol) and iodomethane (1.75 mg, 12.33 mmol) in tetrahydrofuran (25 mL) was added lithium bis(trimethylsilyl)amide (15.0 mL, 15 mmol) dropwise at 0° C., the mixture was stirred at 0° C. to room temperature for 2 h. The reaction was quenched with saturated ammonium chloride solution. The resulting solution was diluted with water, extracted with ethyl acetate, washed with brine and concentrated. The residue was purified by flash chromatography on silica gel eluting with petroleum ether/ethyl acetate (80:20) to afford ethyl 2-(2-chloropyridin-3-yl)-2-methylpropanoate (970 mg, 4.26 mmol, 85% yield) as a yellow solid. LCMS (ESI, m/z): 228.0 [M+H]⁺.

Step 2: 2-(2-chloropyridin-3-yl)-2-methylpropanoic acid

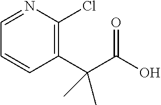

A mixture of ethyl 2-(2-chloropyridin-3-yl)-2-methylpropanoate (950.0 mg, 4.17 mmol) and sodium hydroxide (850.0 mg, 21.25 mmol) in ethanol (20 mL) and water (10 mL) was stirred at 80° C. for 24 h. The ethanol was removed under vacuum. The resulting solution was extracted with ethyl acetate and the aqueous layers were combined. The reaction mixture was adjusted to acid with 1M HCl. The resulting solution was extracted with ethyl acetate and the organic layer was concentrated in vacuum. This resulted in 2-(2-chloropyridin-3-yl)-2-methylpropanoic acid (800 mg, 4.00 mmol, 96% yield) as a white solid. LCMS (ESI, m/z): 199.9 [M+H]⁺.

Step 3: tert-butyl (2-(2-chloropyridin-3-yl)propan-2-yl)carbamate

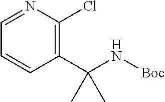 [SMILES: CC(C)(C)OC(=O)NC(C)(C)c1cccnc1Cl]

A mixture of 2-(2-chloropyridin-3-yl)-2-methylpropanoic acid (400.0 mg, 2.00 mmol), triethylamine (600.0 mg, 5.93 mmol) and diphenylphosphoryl azide (830.0 mg, 3.02 mmol) in 2-methyl-2-propanol (10 mL) was stirred at 85° C. for 48 h. The resulting solution was diluted with water and extracted with ethyl acetate. The organic layers were concentrated in vacuum. The residue was purified by flash chromatography on silica gel eluting with petroleum ether/ethyl acetate (7/3) to afford tert-butyl (2-(2-chloropyridin-3-yl)propan-2-yl)carbamate (430.0 mg, 1.58 mmol, 79.3% yield) as a white solid. LCMS (ESI, m/z): 271.2 [M+H]⁺.

Step 4: tert-butyl (2-((tert-butyldimethylsilyl)oxy)ethyl)(2-(2-chloropyridin-3-yl)propan-2-yl)carbamate

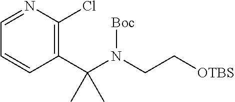 [SMILES: CC(C)(C)OC(=O)N(CCO[Si](C)(C)C(C)(C)C)C(C)(C)c1cccnc1Cl]

To a mixture of tert-butyl (2-(2-chloropyridin-3-yl)propan-2-yl)carbamate (600.0 mg, 2.22 mmol) in N,N-dimethylformamide (10 mL) was added sodium hydride (400.0 mg, 10 mmol) at room temperature and stirred for 1h, then (2-bromoethoxy)-tert-butyldimethylsilane (1.05 g, 4.39 mmol) was added and stirred for 16 h. The resulting solution was quenched with saturated ammonium chloride solution, diluted with water and extracted with ethyl acetate. The organic layers were washed with brine, dried over anhydrous sodium sulfate and concentrated. The residue was purified by flash chromatography on silica gel eluting with petroleum ether/ethyl acetate (80:20) to afford tert-butyl (2-((tert-butyldimethylsilyl)oxy)ethyl)(2-(2-chloropyridin-3-yl)propan-2-yl)carbamate (540 mg, 1.25 mmol, 56.8% yield) as a yellow oil. LCMS (ESI, m/z): 429.4 [M+H]⁺.

Step 5: 2-((2-(2-chloropyridin-3-yl)propan-2-yl)amino)ethan-1-ol

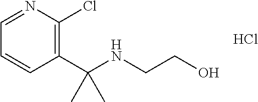

A mixture of tert-butyl (2-((tert-butyldimethylsilyl)oxy)ethyl)(2-(2-chloropyridin-3-yl)propan-2-yl)carbamate (300.0 mg, 0.70 mmol) in 4 M HCl in dioxane (5 mL) was stirred at room temperature for 1 h. The solvent was removed under vacuum. The crude product (300 mg, crude) would be directly used in the next step without purification. LCMS (ESI, m/z): 215.2 [M+H]⁺.

Step 6: 7-(6-(bis(4-methoxybenzyl)amino)-4-methyl-3-(trifluoromethyl)pyridin-2-yl)-6-chloro-5-(2-((2-(2-chloropyridin-3-yl)propan-2-yl)amino)ethoxy)quinazolin-4(3H)-one

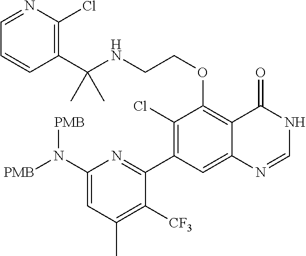

A mixture of 2-((2-(2-chloropyridin-3-yl)propan-2-yl)amino)ethan-1-ol (350.0 mg, 0.57 mmol) and sodium hydride (100.0 mg, 2.50 mmol, 60% purity) in tetrahydrofuran (4 mL) was stirred at 65° C. for 4h. Then 7-(6-(bis(4-methoxybenzyl)amino)-4-methyl-3-(trifluoromethyl)pyridin-2-yl)-6-chloro-5-fluoroquinazolin-4(3H)-one (350.0 mg, 0.65 mmol) was added and stirred for 2 h. The resulting solution was quenched with saturated ammonium chloride solution. The resulting solution was diluted with water and extracted with ethyl acetate. The organic layers were dried over anhydrous sodium sulfate and concentrated in vacuum. The residue was purified by flash chromatography on silica gel eluting with dichloromethane/methanol (93/7) to afford 7-(6-(bis(4-methoxybenzyl)amino)-4-methyl-3-(trifluoromethyl)pyridin-2-yl)-6-chloro-5-(2-((2-(2-chloropyridin-3-yl)propan-2-yl)amino)ethoxy)quinazolin-4(3H)-one (130.0 mg, 0.16 mmol, 28.2% yield) as a white solid. LCMS (ESI, m/z): 807.45 [M+H]⁺

Step 7: 6-(8-chloro-4-(2-(2-chloropyridin-3-yl)propan-2-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

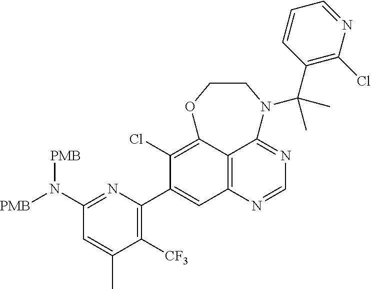

To a mixture of 7-(6-(bis(4-methoxybenzyl)amino)-4-methyl-3-(trifluoromethyl)pyridin-2-yl)-6-chloro-5-(2-((2-(2-chloropyridin-3-yl)propan-2-yl)amino)ethoxy)quinazolin-4(3H)-one (170.0 mg, 0.21 mmol) in chloroform (5 mL) was added N,N-diisopropylethylamine (0.2 mL, 1.16 mmol) and bis(2-oxo-3-oxazolidinyl)phosphinic chloride (100 mg, 0.39 mmol), the mixture was stirred for 2 days at 70° C. The resulting solution was diluted with water, extracted with ethyl acetate, dried over anhydrous sodium sulfate and concentrated. The residue was purified by flash chromatography on silica gel eluting with petroleum ether/ethyl acetate (1/4) to afford 6-(8-chloro-4-(2-(2-chloropyridin-3-yl)propan-2-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (105.0 mg, 0.13 mmol, 63.2% yield) as a yellow solid. LCMS (ESI, m/z): 791.5 [M+H]⁺

Step 8: 6-(8-chloro-4-(2-(2-((4-methoxybenzyl)amino)pyridin-3-yl)propan-2-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

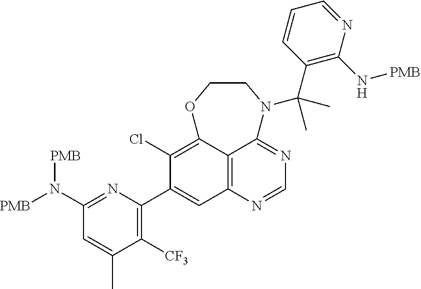

A mixture of 6-(8-chloro-4-(2-(2-chloropyridin-3-yl)propan-2-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (50.0 mg, 0.06 mmol), 4-methoxybenzylamine (20.0 mg, 0.15 mmol), tris(dibenzylideneacetone)dipalladium (7.0 mg, 0.01 mmol), 1.1′-Binaphthyl-2.2′-diphenyl phosphine (8.0 mg, 0.01 mmol) and sodium tert-butoxide (20.0 mg, 0.21 mmol) in 1,4-dioxane (2 mL) was stirred at 100° C. for 1 h under nitrogen. The resulting solution was diluted with water and extracted with ethyl acetate. The combined organic layers were dried over anhydrous sodium sulfate and concentrated under vacuum. The residue was purified by flash chromatography on silica gel eluting with petroleum ether/ethyl acetate (2/1) to afford 6-(8-chloro-4-(2-(2-((4-methoxybenzyl)amino)pyridin-3-yl)propan-2-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (30.0 mg, 0.03 mmol, 53.2% yield) as a yellow solid. LCMS (ESI, m/z): 890.3 [M+H]⁺

Step 9: 6-(4-(2-(2-aminopyridin-3-yl)propan-2-yl)-8-chloro-5,6-dihydro-4H-[1,4]oxazepino[5,6,7-de]quinazolin-9-yl)-4-methyl-5-(trifluoromethyl)pyridin-2-amine

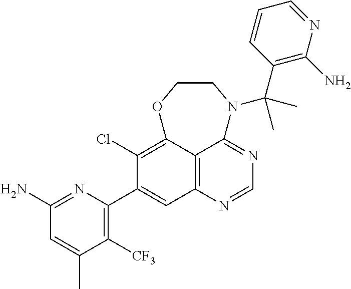 [SMILES: Cc1cc(N)nc(-c2cc3ncnc4c3c(c2Cl)OCCN4C(C)(C)c2cccnc2N)c1C(F)(F)F]

A mixture of 6-(8-chloro-4-(2-(2-((4-methoxybenzyl)amino)pyridin-3-yl)propan-2-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (30.0 mg, 0.03 mmol) in trifluoroacetic acid (2 mL) was stirred at 50° C. for 24 h. The solvent was removed under vacuum. The crude product was purified by Prep-HPLC with the following conditions: Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 30% B to 50% B in 10 min, 50% B; Wave Length: 254/220 nm; RT: 9.67 min. This resulted in 6-(4-(2-(2-aminopyridin-3-yl)propan-2-yl)-8-chloro-5,6-dihydro-4H-[1,4]oxazepino[5,6,7-de]quinazolin-9-yl)-4-methyl-5-(trifluoromethyl)pyridin-2-amine (1.6 mg, 0.003 mmol, 9% yield). LCMS (ESI, m/z): 530.0 [M+H]⁺.

Example 27: ¹H NMR (300 MHz, DMSO-cd, ppm) δ 8.22 (s, 1H), 7.80 (dd, J=4.8, 1.6 Hz, 1H), 7.60 (dd, J=7.7, 1.7 Hz, 1H), 7.30 (s, 1H), 6.77 (s, 2H), 6.64 (dd, J=7.6, 4.8 Hz, 1H), 6.46 (s, 1H), 5.76 (s, 2H), 4.73-4.48 (m, 2H), 3.97-3.65 (m, 2H), 2.36 (q, J=2.1 Hz, 3H), 1.84 (d, J=9.0 Hz, 6H).

Example 28: 6-(4-((2-aminopyridin-3-yl)(cyclopropyl)methyl)-8-chloro-5,6-dihydro-4H-[1,4]oxazepino[5,6,7-de]quinazolin-9-yl)-4-methyl-5-(trifluoromethyl)pyridin-2-amine

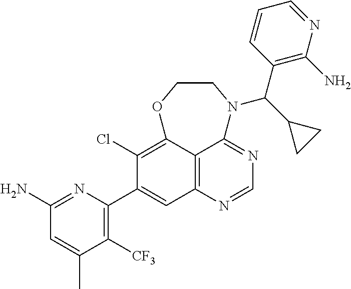 [SMILES: Cc1cc(N)nc(-c2cc3ncnc4c3c(c2Cl)OCCN4C(c2cccnc2N)C2CC2)c1C(F)(F)F]

### Synthetic Route

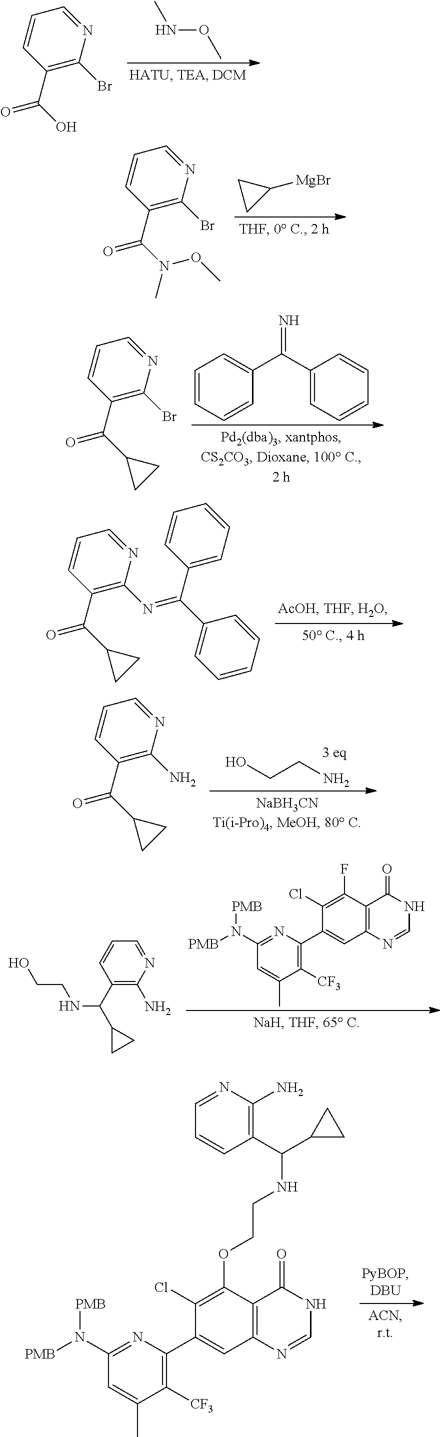

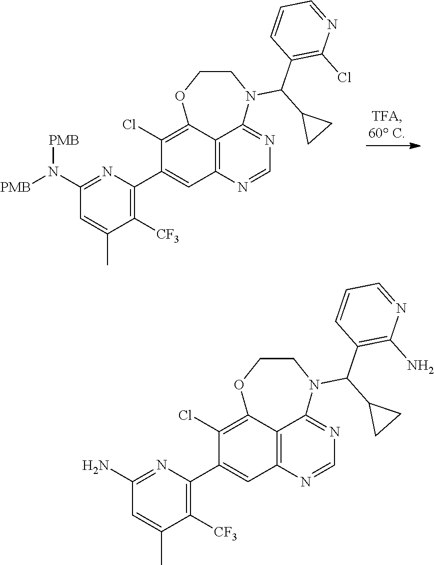

Step 1: 2-bromo-N-methoxy-N-methylnicotinamide

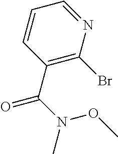

A mixture of 2-bromonicotinic acid (14.0 g, 69.3 mmol), N-methoxymethanamine (5.5 g, 90.05 mmol), 2-(7-Azabenzotrazol-1-yl)-N,N,N′,N′-tetramethyluronium hexafluorophosphate (28.0 g, 73.64 mmol) and N,N-diisopropylethylamine (36.39 mL, 208.91 mmol) in dichloromethane (150 mL) was stirred at room temperature for 2 h. The resulting solution was diluted with dichloromethane, washed with brine, dried over anhydrous sodium sulfate and concentrated. The residue was purified by flash chromatography on silica gel eluting with petroleum ether/ethyl acetate (7/3) to afford 2-bromo-N-methoxy-N-methylnicotinamide (15.00 g, 61.20 mmol, 88.3% yield) as a white solid. LCMS (ESI, m/z): 245.1 [M+H]⁺.

Step 2: (2-bromopyridin-3-yl)(cyclopropyl)methanone

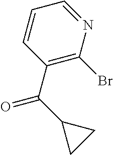 [SMILES: O=C(c1cccnc1Br)C1CC1]

To a mixture of 2-bromo-N-methoxy-N-methylnicotinamide (13.00 g, 53.05 mmol) in tetrahydrofuran (130 mL) was added cyclopropyl magnesium bromide (53.0 mL, 106.69 mmol, 2M) and stirred for 2 h at 0° C. The reaction was quenched with saturated ammonium chloride solution and concentrated under vacuum. The resulting solution was diluted with water and extracted with ethyl acetate. The combined organic layers were dried over anhydrous sodium sulfate and concentrated under vacuum. The residue was purified by flash chromatography on silica gel eluting with petroleum ether/ethyl acetate (3/7) to afford (2-bromopyridin-3-yl)(cyclopropyl)methanone (6.00 g, 26.54 mmol, 50% yield) as light yellow oil. LCMS (ESI, m/z): 226.1 [M+H]⁺.

Step 3: cyclopropyl(2-((diphenylmethylene)amino)pyridin-3-yl)methanone

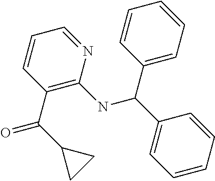 [SMILES: O=C(c1cccnc1NC(c1ccccc1)c1ccccc1)C1CC1]

A mixture of (2-bromopyridin-3-yl)(cyclopropyl)methanone (5.1 g, 22.56 mmol), benzophenone imine (5.74 mL, 34.21 mmol), tris(dibenzylideneacetone)dipalladium (2.1 g, 2.29 mmol), 4,5-bis(diphenylphosphino)-9,9-dimethylxanthene (1.32 g, 2.28 mmol) and cesium carbonate (22 g, 67.53 mmol) in 1,4-dioxane (60 mL) was stirred at 100° C. for 2 h under nitrogen. The resulting solution was diluted with ethyl acetate, washed with brine, dried over anhydrous sodium sulfate and concentrated. The residue was purified by flash chromatography on silica gel eluting with petroleum ether/ethyl acetate (1/1) to afford cyclopropyl(2-((diphenylmethylene)amino)pyridin-3-yl)methanone (3.00 g, 9.19 mmol, 40.7% yield) as a yellow solid. LCMS (ESI, m/z): 327.4 [M+H]⁺.

Step 4: (2-amino-3-pyridyl)-cyclopropyl-methanone

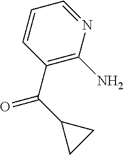 [SMILES: Nc1ncccc1C(=O)C1CC1]

To a mixture of cyclopropyl(2-((diphenylmethylene)amino)pyridin-3-yl)methanone (3.00 g, 9.19 mmol) in tetrahydrofuran (10 mL) was added acetic acid (10 mL) and water (1 mL), the mixture was stirred for 2 h at 50° C. The solvent was concentrated under vacuum, diluted with ethyl acetate, adjusted PH=7.0 with saturated sodium carbonate solution, washed with brine, dried over anhydrous sodium sulfate and concentrated. The residue was purified by flash chromatography on silica gel eluting with petroleum ether/ethyl acetate (1/1) to afford (2-amino-3-pyridyl)-cyclopropyl-methanone (1.40 g, 8.63 mmol, 93.9% yield) as a white solid. LCMS (ESI, m/z): 163.2 [M+H]⁺.

Step 5: 2-(((2-aminopyridin-3-yl)(cyclopropyl)methyl)amino)ethan-1-ol

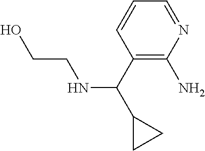 [SMILES: Nc1ncccc1C(NCCO)C1CC1]

To a mixture of (2-amino-3-pyridyl)-cyclopropyl-methanone (500.0 mg, 3.08 mmol), 2-aminoethanol (375.0 mg, 6.14 mmol) in methyl alcohol (6 mL) was added titanium tetraisopropanolate (875.0 mg, 3.08 mmol), the mixture was stirred for 3 h at 80° C. Then sodium cyanoborohydride (375.0 mg, 5.97 mmol) was added, the mixture was stirred for another 3 h at 80° C. The solvent was concentrated under vacuum. The resulting residue was purified by reverse phase chromatography (acetonitrile/0.1% NH₄HCO₃ in water) to afford 2-(((2-aminopyridin-3-yl)(cyclopropyl)methyl)amino)ethan-1-ol (200.0 mg, 0.96 mmol, 31.3% yield) as a white solid. LCMS (ESI, m/z): 208.3 [M+H]⁺.

Step 6: 5-(2-(((2-aminopyridin-3-yl)(cyclopropyl)methyl)amino)ethoxy)-7-(6-(bis(4-methoxybenzyl)amino)-4-methyl-3-(trifluoromethyl)pyridin-2-yl)-6-chloroquinazolin-4(3H)-one

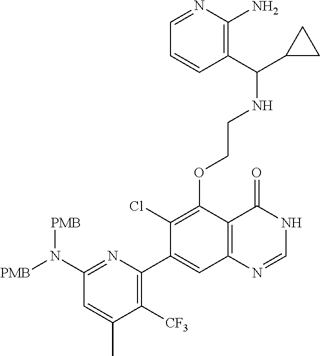

To a mixture of 2-(((2-aminopyridin-3-yl)(cyclopropyl)methyl)amino)ethan-1-ol (100.0 mg, 0.48 mmol) in tetrahydrofuran (2 mL) was added sodium hydride (66.8 mg, 1.67 mmol, 60% purity) and stirred for 5 min at 0° C. Then 7-[6-[bis[(4-methoxyphenyl)methyl]amino]-4-methyl-3-(trifluoromethyl)-2-pyridyl]-6-chloro-5-fluoro-3H-quinazolin-4-one (200.0 mg, 0.33 mmol) was added and stirred for 2 h at 70° C. The reaction was quenched with saturated ammonium chloride solution. The resulting solution diluted with water, extracted with ethyl acetate, dried over anhydrous sodium sulfate and concentrated. The residue was purified by flash chromatography on silica gel eluting with petroleum ether/ethyl acetate (1/4) to afford 5-(2-(((2-aminopyridin-3-yl)(cyclopropyl)methyl)amino)ethoxy)-7-(6-(bis(4-methoxybenzyl)amino)-4-methyl-3-(trifluoromethyl)pyridin-2-yl)-6-chloroquinazolin-4(3H)-one (80.0 mg, 0.1 mmol, 30.6% yield) as a light yellow solid. LCMS (ESI, m/z): 800.3[M+H]⁺.

Step 7: 6-(4((2-aminopyridin-3-yl)(cyclopropyl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

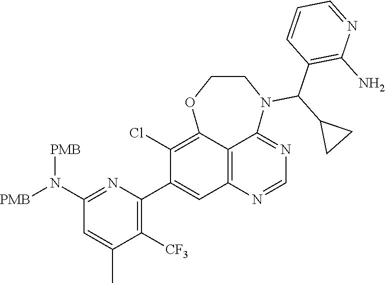

A mixture of 5-(2-(((2-aminopyridin-3-yl)(cyclopropyl)methyl)amino)ethoxy)-7-(6-(bis(4-methoxybenzyl)amino)-4-methyl-3-(trifluoromethyl)pyridin-2-yl)-6-chloroquinazolin-4(3H)-one (80 mg, 0.1 mmol), benzotriazole-1-yl-oxytripyrrolidinophosphonium hexafluorophosphate (56.0 mg, 0.11 mmol) and 1,8-diazabicyclo[5.4.0]undec-7-ene (0.05 mL, 0.32 mmol) in acetonitrile (2 mL) was stirred at room temperature for 2 h. The resulting solution was diluted with water, extracted with ethyl acetate, dried over anhydrous sodium sulfate and concentrated. The residue was purified by flash chromatography on silica gel eluting with dichloromethane/methanol (20/1) to afford 6-(4-((2-aminopyridin-3-yl)(cyclopropyl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (70.0 mg, 0.09 mmol, 89.5% yield) as a white solid. LC-MS: (ESI, m/z): 782.3 [M+H]⁺.

Step 8:6-(4-((2-aminopyridin-3-yl)(cyclopropyl)methyl)-8-chloro-5,6-dihydro-4H-[1,4]oxazepino[5,6,7-de]quinazolin-9-yl)-4-methyl-5-(trifluoromethyl)pyridin-2-amine

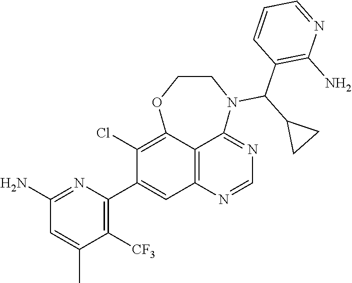 [SMILES: Cc1cc(N)nc(-c2cc3ncnc4c3c(c2Cl)OCCN4C(c2cccnc2N)C2CC2)c1C(F)(F)F]

A mixture of 6-(4-((2-aminopyridin-3-yl)(cyclopropyl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (60.0 mg, 0.08 mmol) in 2,2,2-trifluoroacetic acid (1 mL) was stirred at 50° C. for 4 h. The solvent was removed under vacuum. The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN: Flow rate: 60 mL/min; Gradient: 33% B to 63% B in 7 min; Wave Length: 254 nm; RT: 6.5 min) to afford 6-(4-((2-aminopyridin-3-yl)(cyclopropyl)methyl)-8-chloro-5,6-dihydro-4H-[1,4]oxazepino[5,6,7-de]quinazolin-9-yl)-4-methyl-5-(trifluoromethyl)pyridin-2-amine (10.3 mg, 0.019 mmol, 24.8% yield). LC-MS: (ESI, m/z): 542.4 [M+H]⁺.

Example 28: ¹H NMR (400 MHz, Methanol-d₄) δ 8.45 (s, 1H), 8.04 (dd, J=7.7, 1.7 Hz, 1H), 7.95 (dt, J=5.3, 1.6 Hz, 1H), 7.28 (s, 1H), 6.83-6.76 (m, 1H), 6.59 (s, 1H), 5.85 (d, J=10.2 Hz, 1H), 4.66 (td, J=12.2, 6.4 Hz, 1H), 4.40 (td, J=11.2, 6.8 Hz, 1H), 4.00-3.87 (m, 1H), 3.66 (dd, J=16.0, 6.6 Hz, 1H), 2.49-2.43 (m, 3H), 1.81-1.79 (m, 1H), 0.88-0.83 (m, 1H), 0.71-0.67 (s, 1H), 0.59-0.56 (m, 1H), 0.43-0.39 (m, 1H).

Example 29: 6-(8-chloro-4-((5-(methylthio)pyridin-3-yl)methyl)-5,6-dihydro-4H-[1,4]oxazepino[5,6,7-de]quinazolin-9-yl)-4-methyl-5-(trifluoromethyl)pyridin-2-amine

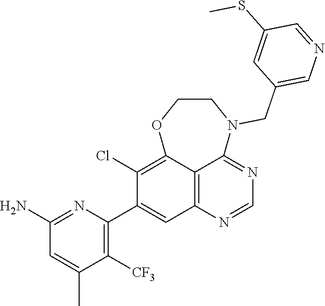 [SMILES: CSc1cncc(CN2CCOc3c(Cl)c(-c4nc(N)cc(C)c4C(F)(F)F)cc4ncnc2c34)c1]

### Synthetic Route

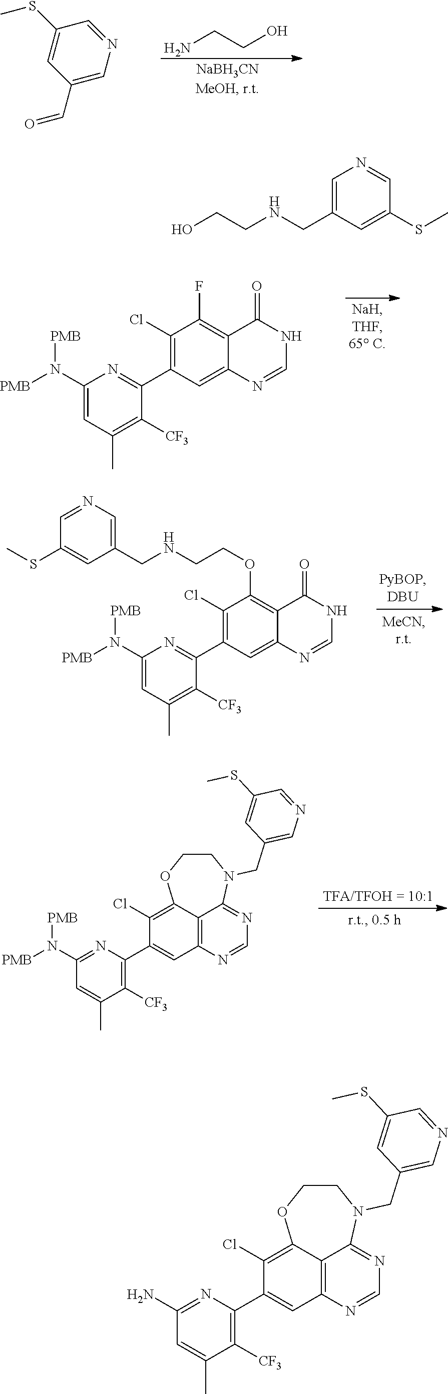

Step 1: 2-(((5-(methylthio)pyridin-3-yl)methyl)amino)ethan-1-ol

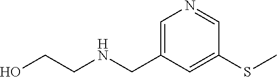

A solution of 5-(methylthio)nicotinaldehyde (400.0 mg, 2.61 mmol), 2-aminoethan-1-ol (287.1 mg, 4.7 mmol) and acetic acid (15.6 mg, 0.26 mmol) in methyl alcohol (6 mL) was stirred at 25° C. for 1 hour. Then sodium cyanoborohydride (492.2 mg, 7.83 mmol) was added and stirred at 25° C. for 21 hours. After completion, the reaction mixture was quenched with water, concentrated under vacuum and purified by flash chromatography on reverse-phase column eluting with water/acetonitrile (7/3) to afford 2-(((5-(methylthio)pyridin-3-yl)methyl)amino)ethan-1-ol (380.4 mg, 1.88 mmol, 71.9% yield) as a yellow oil. LC-MS: (ESI, m/z): 199.1 [M+H]⁺

Step 2: 7-(6-(bis(4-methoxybenzyl)amino)-4-methyl-3-(trifluoromethyl)pyridin-2-yl)-6-chloro-5-(2-(((5-(methylthio)pyridin-3-yl)methyl)amino)ethoxy)quinazolin-4(3H)-one

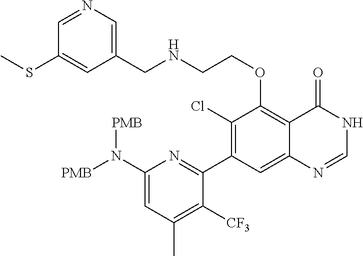

To a solution of 2-(((5-(methylthio)pyridin-3-yl)methyl)amino)ethan-1-ol (116.5 mg, 0.59 mmol) in tetrahydrofuran (3 mL) was added sodium hydride (78.3 mg, 1.96 mmol) at 0° C. and stirred at 25° C. for 30 min. Then 7-(6-(bis(4-methoxybenzyl)amino)-4-methyl-3-(trifluoromethyl)pyridin-2-yl)-6-chloro-5-fluoroquinazolin-4(3H)-one (300.0 mg, 0.49 mmol) was added at 25° C. and stirred at 65° C. for 1 hour. After completion, the reaction was quenched with 1M hydrochloric acid and concentrated under vacuum. The residue was purified by flash chromatography on reversed phase column eluting with water/acetonitrile (6/4) to afford 7-(6-(bis(4-methoxybenzyl)amino)-4-methyl-3-(trifluoromethyl)pyridin-2-yl)-6-chloro-5-(2-(((5-(methylthio)pyridin-3-yl)methyl)amino)ethoxy)quinazolin-4(3H)-one (250.3 mg, 0.27 mmol, 56.2% yield) as a yellow solid. LC-MS: (ESI, m/z): 791.2 [M+H]⁺

Step 3: 6-(8-chloro-4-((5-(methylthio)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

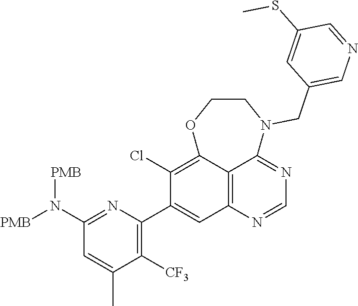

To a solution of 7-(6-(bis(4-methoxybenzyl)amino)-4-methyl-3-(trifluoromethyl)pyridin-2-yl)-6-chloro-5-(2-(((5-(methylthio)pyridin-3-yl)methyl)amino)ethoxy)quinazolin-4(3H)-one (230.0 mg, 0.25 mmol) and 1,8-diazabicyclo[5.4.0]undec-7-ene (153.8 mg, 1.01 mmol) in acetonitrile (3 mL) was added benzotriazole-1-yl-oxytripyrrolidinophosphonium hexafluorophosphate (171.1 mg, 0.33 mmol) and stirred at 25° C. for 1 hour. After completion, the solvent was concentrated under vacuum. The residue was purified by flash chromatography on reversed phase column eluting with water/acetonitrile (7/3) to afford 6-(8-chloro-4-((5-(methylthio)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (175.5 mg, 0.21 mmol, 83.2% yield) as an orange solid. LC-MS: (ESI, m/z): 773.3 [M+H]⁺

Step 4: 6-(8-chloro-4-((5-(methylthio)pyridin-3-yl)methyl)-5,6-dihydro-4H-[1,4]oxazepino[5,6,7-de]quinazolin-9-yl)-4-methyl-5-(trifluoromethyl)pyridin-2-amine

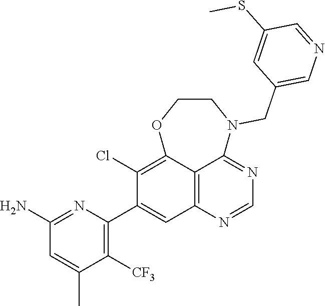 [SMILES: CSc1cncc(CN2CCOc3c(Cl)c(-c4nc(N)cc(C)c4C(F)(F)F)cc4ncnc2c34)c1]

A solution of 6-(8-chloro-4-((5-(methylthio)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (150.0 mg, 0.19 mmol) in trifluoroacetic acid (5 mL, 64.9 mmol) and trifluoromethanesulfonic acid (0.5 mL, 5.65 mmol) was stirred at 25° C. for 1 hour. After completion, the reaction mixture was concentrated under vacuum and diluted with N,N-dimethylformamide, adjusted to pH >7 with N,N-diisopropylethylamine and purified by flash chromatography on reverse-phase column eluting with water/acetonitrile (7/3) to afford crude product. The product was further purified by Prep-HPLC with the following conditions: (Column: XBridge Prep OBD C18 Column, 30*150 mm, 5 μm; Mobile Phase A: water (10 mmol/L NH₄HCO₃), Mobile Phase B: acetonitrile: Flow rate: 60 mL/min; Gradient: 22% B to 52% B in 7 min; Wave Length: 254 nm; RT: 6.5 min) to afford 6-(8-chloro-4-((5-(methylthio)pyridin-3-yl)methyl)-5,6-dihydro-4H-[1,4]oxazepino[5,6,7-de]quinazolin-9-yl)-4-methyl-5-(trifluoromethyl)pyridin-2-amine (24.0 mg, 0.04 mmol, 23.2% yield). LC-MS: (ESI, m/z): 533.2 [M+H]⁺

Example 29: ¹H NMR (400 MHz, Methanol-d4, ppm) δ 8.47 (s, 1H), 8.37 (s, 2H), 7.80 (s, 1H), 7.31 (s, 1H), 6.59 (s, 1H), 5.26-5.15 (m, 2H), 4.67-4.65 (m, 2H), 4.05-4.03 (m, 2H), 2.53 (s, 3H), 2.46 (d, J=1.6 Hz, 3H). LC-MS: (ESI, m/z): 533.2 [M+H]⁺

Example 30: 6-(4-((1H-imidazol-5-yl)methyl)-8-chloro-5,6-dihydro-4H-[1,4]oxazepino[5,6,7-de]quinazolin-9-yl)-4-methyl-5-(trifluoromethyl)pyridin-2-amine

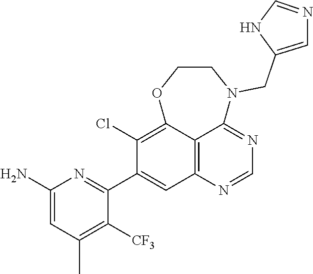 [SMILES: Cc1cc(N)nc(-c2cc3ncnc4c3c(c2Cl)OCCN4Cc2cnc[nH]2)c1C(F)(F)F]

### Synthetic Route

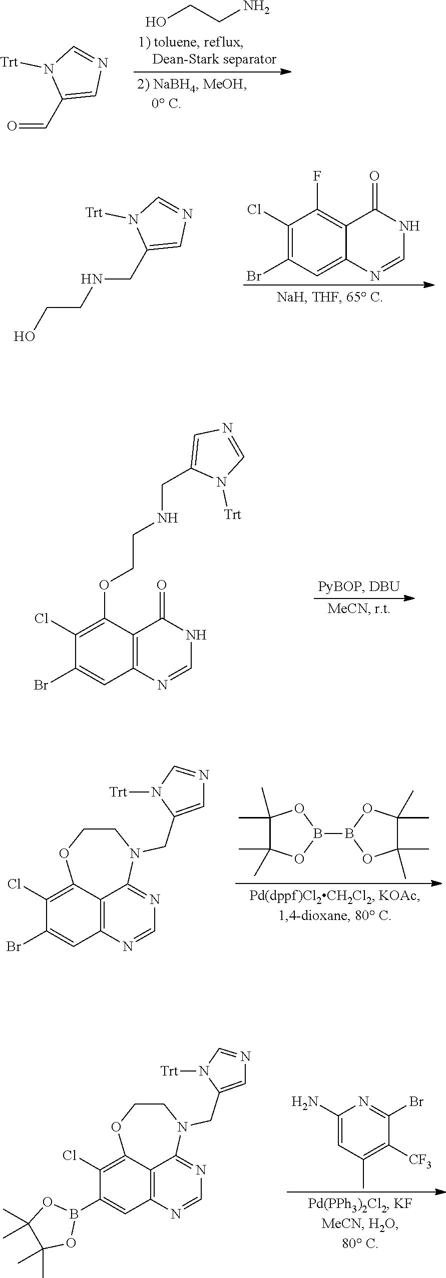

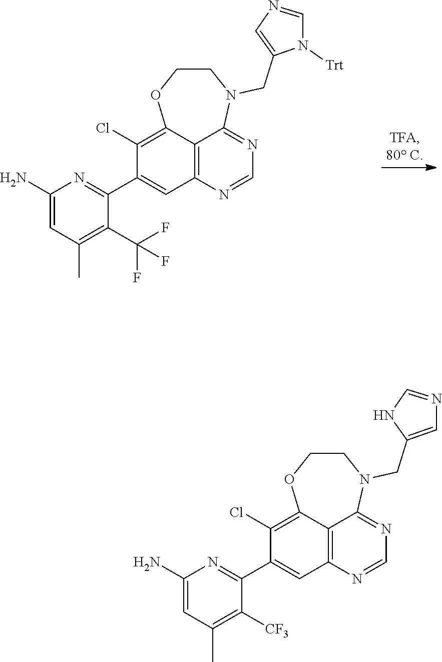

Step 1: 2-(((1-trityl-1H-imidazol-5-yl)methyl)amino)ethan-1-ol

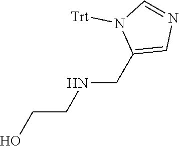

In Dean-Stark separator, a solution of 3-tritylimidazole-4-carbaldehyde (5.00 g, 14.78 mmol) and 2-aminoethanol (902.4 mg, 14.7 mmol) in toluene (200 mL) was stirred for 6 hours at 130° C. The solvent was concentrated under vacuum. The mixture was dissolved in methyl alcohol (150 mL), and sodium borohydride (1.12 g, 29.55 mmol) was added and stirred at 0° C. for 3 hours. The resulting solution was diluted with water and extracted with ethyl acetate. The organic layers were dried over anhydrous sodium sulfate and concentrated under vacuum to afford 2-(((1-trityl-1H-imidazol-5-yl)methyl)amino)ethan-1-ol (6.00 g, 14.0 mmol, 95.3% yield) as a yellow solid. LC-MS: (ESI, m/z): 384.2 [M+H]⁺.

Step 2: 7-bromo-6-chloro-5-(2-(((1-trityl-1H-imidazol-5-yl)methyl)amino)ethoxy)quinazolin-4(3H)-one

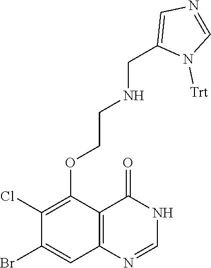

To a solution of 2-(((1-trityl-1H-imidazol-5-yl)methyl)amino)ethan-1-ol (5.53 g, 14.42 mmol) in tetrahydrofuran (10 mL) was added sodium hydride (1.15 g, 28.83 mmol, 60% purity) and stirred at 0° C. for 0.5 hour. Then 7-bromo-6-chloro-5-fluoro-3H-quinazolin-4-one (2.00 g, 7.21 mmol) was added and stirred at 65° C. for 1 hour. After completion, the reaction was quenched with saturated ammonium chloride solution. The combined organic layers were dried over anhydrous sodium sulfate and concentrated under vacuum. The residue was purified by flash chromatography on silica gel eluting with methanol/dichloromethane (1/10) to afford 7-bromo-6-chloro-5-(2-(((1-trityl-1H-imidazol-5-yl)methyl)amino)ethoxy)quinazolin-4(3H)-one (1.45 g, 1.85 mmol, 25.7% yield) as a brown solid. LC-MS: (ESI, m/z): 640.1 [M+H]⁺

Step 3: 9-bromo-8-chloro-4-((1-trityl-1H-imidazol-5-yl)methyl)-5,6-dihydro-4H-[1,4]oxazepino[5,6,7-de]quinazoline

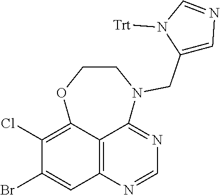

To a solution of 7-bromo-6-chloro-5-(2-(((1-trityl-1H-imidazol-5-yl)methyl)amino)ethoxy)quinazolin-4(3H)-one (1.40 g, 2.18 mmol) and 1,8-diazabicyclo[5.4.0]undec-7-ene (996.0 mg, 6.5 mmol) in acetonitrile (10 mL) was added benzotriazole-1-yl-oxytripyrrolidinophosphonium hexafluorophosphate (1.7 g, 3.28 mmol), the mixture was stirred at 25° C. for 1 hour. The resulting solution was diluted with ethyl acetate and washed with water. The organic layers were dried over anhydrous sodium sulfate and concentrated under vacuum. The residue was purified by flash chromatography on silica gel eluting with methanol/dichloromethane (1/40) to afford 9-bromo-8-chloro-4-((1-trityl-1H-imidazol-5-yl)methyl)-5,6-dihydro-4H-[1,4]oxazepino[5,6,7-de]quinazoline (590.0 mg, 0.8 mmol, 39.5% yield) as a brown solid. LC-MS: (ESI, m/z): 622.1 [M+H]⁺

Step 4: 8-chloro-9-(4,4,5,5-tetramethyl-1,3,2-dioxaborolan-2-yl)-4-((1-trityl-1H-imidazol-5-yl)methyl)-5,6-dihydro-4H-[1,4]oxazepino[5,6,7-de]quinazoline

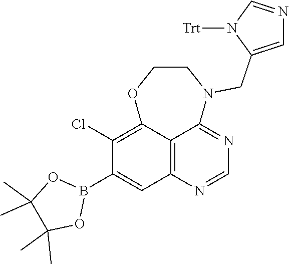

Under nitrogen, a mixture of 9-bromo-8-chloro-4-((1-trityl-1H-imidazol-5-yl)methyl)-5,6-dihydro-4H-[1,4]oxazepino[5,6,7-de]quinazoline (560.0 mg, 0.9 mmol), bis(pinacolato)diboron (456.5 mg, 1.8 mmol), [1,1-bis(diphenylphosphino)ferrocene]dichloropalladium(II) (66.6 mg, 0.09 mmol) and potassium acetate (176.2 mg, 1.8 mmol) in 1,4-dioxane (10 mL) was stirred at 80° C. for 3 hours. The reaction mixture was concentrated under vacuum. The mixture was filtered and washed with dichloromethane. The filtrate was concentrated under reduced pressure. The crude product (1.0 g, crude) was used directly without further purification. LC-MS: (ESI, m/z): 670.3 [M+H]⁺

Step 5: 6-(8-chloro-4-((1-trityl-1H-imidazol-5-yl)methyl)-5,6-dihydro-4H-[1,4]oxazepino[5,6,7-de]quinazolin-9-yl)-4-methyl-5-(trifluoromethyl)pyridin-2-amine

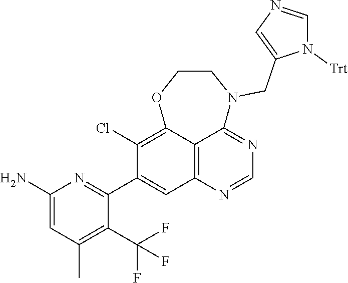

Under nitrogen, a solution of 8-chloro-9-(4,4,5,5-tetramethyl-1,3,2-dioxaborolan-2-yl)-4-((1-trityl-1H-imidazol-5-yl)methyl)-5,6-dihydro-4H-[1,4]oxazepino[5,6,7-de]quinazoline (1.0 g crude), 6-bromo-4-methyl-5-(trifluoromethyl)pyridin-2-amine (243.6 mg, 0.9 mmol), potassium fluoride (277.0 mg, 4.7 mmol) and bis(triphenylphosphine)palladium(II) chloride (167.6 mg, 0.2 mmol) in acetonitrile (10 mL) and water (2 mL) was stirred at 80° C. for 2 hours. After completion, the reaction mixture was diluted with water. The resulting solution was extracted with ethyl acetate and the organic layers were dried over anhydrous sodium sulfate and concentrated under vacuum. The residue was purified by flash chromatography on silica gel eluting with dichloromethane/methanol (1/15) to afford 6-(8-chloro-4-((1-trityl-1H-imidazol-5-yl)methyl)-5,6-dihydro-4H-[1,4]oxazepino[5,6,7-de]quinazolin-9-yl)-4-methyl-5-(trifluoromethyl)pyridin-2-amine (438.0 mg, 0.5 mmol, 24.5% yield) as a brown solid. LC-MS: (ESI, m/z): 718.2 [M+H]⁺

Step 6: 6-(4-((1H-imidazol-5-yl)methyl)-8-chloro-5,6-dihydro-4H-[1,4]oxazepino[5,6,7-de]quinazolin-9-yl)-4-methyl-5-(trifluoromethyl)pyridin-2-amine

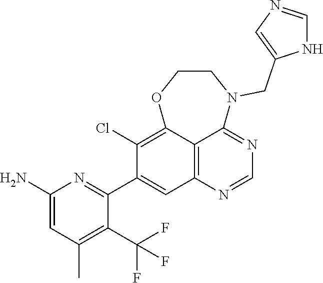 [SMILES: Cc1cc(N)nc(-c2cc3ncnc4c3c(c2Cl)OCCN4Cc2cnc[nH]2)c1C(F)(F)F]

A solution of 6-(8-chloro-4-((1-trityl-1H-imidazol-5-yl)methyl)-5,6-dihydro-4H-[1,4]oxazepino[5,6,7-de]quinazolin-9-yl)-4-methyl-5-(trifluoromethyl)pyridin-2-amine (400.0 mg, 0.5 mmol) in trifluoroacetic acid (6 mL, 0.5 mmol) was stirred at 60° C. for 1 hour. After completion, the solvent was concentrated under vacuum. The crude product was purified by Prep-HPLC with the following conditions: (Column: XBridge Prep OBD C18 Column, 30×150 mm 5 um; Mobile Phase A: Water (10 mmol/L NH₄HCO₃), Mobile Phase B: ACN; Flow rate: 60 mL/min; Gradient: 28 B to 58 B in 7 min; 254 nm; RT: 6.20 min) to afford 6-(4-((1H-imidazol-5-yl)methyl)-8-chloro-5,6-dihydro-4H-[1,4]oxazepino[5,6,7-de]quinazolin-9-yl)-4-methyl-5-(trifluoromethyl)pyridin-2-amine (38.5 mg, 0.08 mmol, 14.5% yield). LC-MS: (ESI, m/z): 476.2 [M+H]⁺

Example 30: ¹H NMR (300 MHz, DMSO-d₆, ppm) δ 11.9 (s, 1H), 8.42 (s, 1H), 7.57 (d, J=1.2 Hz, 1H), 7.14 (s, 1H), 7.05 (s, 1H), 6.74 (s, 2H), 6.43 (s, 1H), 5.01-4.91 (m, J=14.7 Hz, 2H), 4.64-4.45 (m, 2H), 3.98-3.89 (m, 2H), 2.33 (d, J=2.3 Hz, 3H).

Example 31: 6-(4-((2-amino-5-fluoropyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

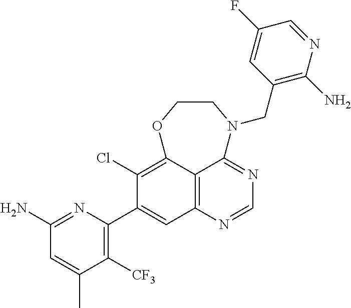 [SMILES: Cc1cc(N)nc(-c2cc3ncnc4c3c(c2Cl)OCCN4Cc2cc(F)cnc2N)c1C(F)(F)F]

### Synthetic Route

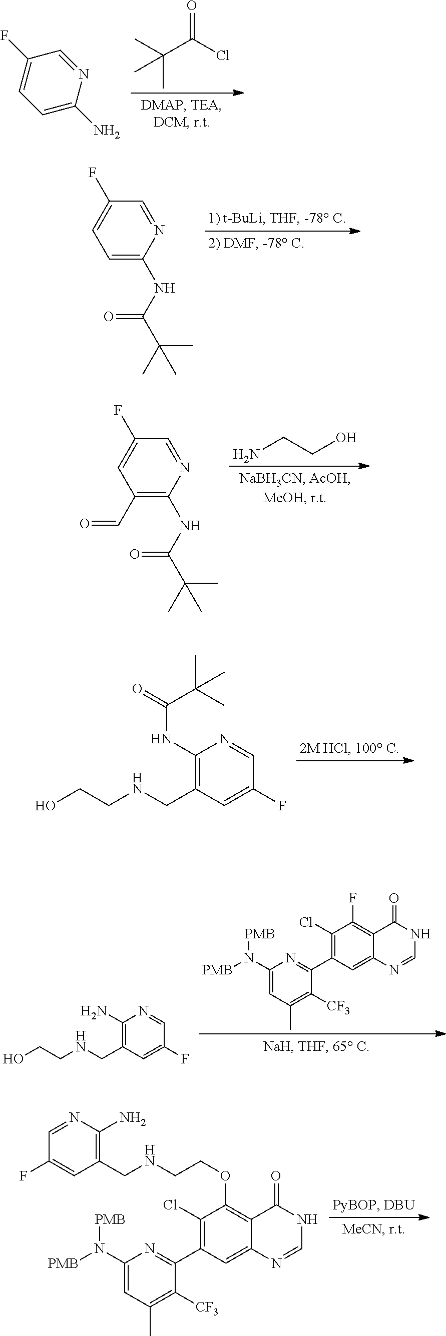

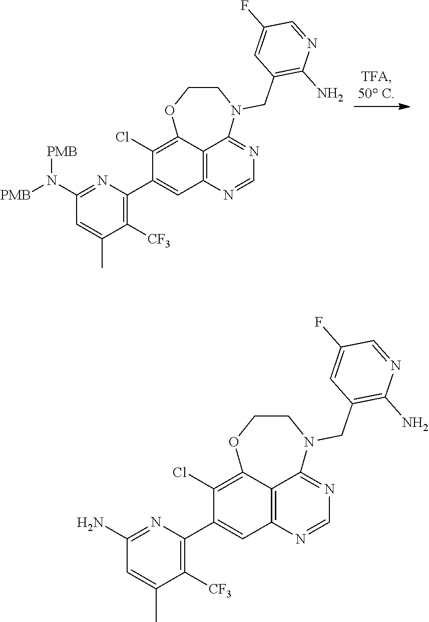

Step 1: N-(5-fluoropyridin-2-yl)pivalamide

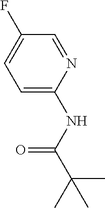

A solution of 5-fluoro-2-pyridinamine (10.00 g, 89.20 mmol), pivaloyl chloride (13.18 mL, 107.04 mmol) and triethylamine (27.03 g, 267.59 mmol) in dichloromethane (100 mL) was stirred at 25° C. for 5 hours. After completion, the reaction was quenched with saturated sodium bicarbonate solution. The resulting solution was extracted with dichloromethane and the organic layers were concentrated under vacuum. The residue was purified by flash chromatography on silica gel eluting with petroleum ether/ethyl acetate (311) to afford N-(5-fluoropyridin-2-yl)pivalamide (11.00 g, 56.0 mmol) as a yellow oil. LC-MS: (ESI, m/z): 197.1 [M+H]⁺

Step 2: N-(5-fluoro-3-formylpyridin-2-yl)pivalamide

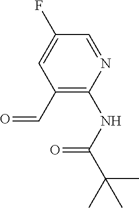 [SMILES: CC(C)(C)C(=O)Nc1ncc(F)cc1C=O]

To a solution of N-(5-fluoropyridin-2-yl)pivalamide (11.0 g, 56.0 mmol) in tetrahydrofuran (80 mL) was added tert-butyl lithium (37 ml, 56.0 mmol, 1.5M) at −78° C., the mixture was stirred at −78° C. for 1 hour. Then N,N-dimethylformamide (4.0 g, 56.0 mmol) was added and stirred at −78° C. for another 2 hours. After completion, the reaction was quenched with saturated ammonium chloride solution. Tetrahydrofuran was concentrated under vacuum, diluted with dichloromethane, washed with water, dried over anhydrous sodium sulfate and concentrated under vacuum. The residue was purified by flash chromatography on silica gel eluting with petroleum ether/ethyl acetate (2/1) to afford N-(5-fluoro-3-formylpyridin-2-yl)pivalamide (3.10 g, 13.7 mmol). LC-MS: (ESI, m/z): 225.1 [M+H]⁺

Step 3: N-(5-fluoro-3-(((2-hydroxyethyl)amino)methyl)pyridin-2-yl)pivalamide

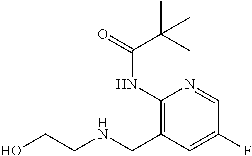 [SMILES: CC(C)(C)C(=O)Nc1ncc(F)cc1CNCCO]

A solution of N-(5-fluoro-3-formylpyridin-2-yl)pivalamide (1.00 g, 4.46 mmol), acetic acid (0.11 g, 1.87 mmol) and 2-aminoethanol (272.4 mg, 4.4 mmol) in methanol was stirred at 25° C. for 6 hours. Then sodium borohydride (254.2 mg, 6.6 mmol) was added stirred at 25° C. for 3 hours. After completion, the reaction was quenched with water and concentrated under vacuum. The residue was purified by flash chromatography on silica gel eluting with methanol/dichloromethane (1/25) to afford N-(5-fluoro-3-(((2-hydroxyethyl)amino)methyl)pyridin-2-yl)pivalamide (505.0 mg, 1.7 mmol, 41.6% yield) as a yellow oil. LC-MS: (ESI, m/z): 270.2 [M+H]⁺

Step 4: 2-(((2-amino-5-fluoropyridin-3-yl)methyl)amino)ethan-1-ol. hydrogen chloride

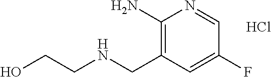 [SMILES: Nc1ncc(F)cc1CNCCO ; Cl]

A solution of N-(5-fluoro-3-(((2-hydroxyethyl)amino)methyl)pyridin-2-yl)pivalamide (500.0 mg, 1.6 mmol) in hydrochloric acid aqueous solution (10 mL, 2N) was stirred at 100° C. for 3 hours. After completion, the solvent was concentrated under vacuum. The residue was purified by flash chromatography on silica gel eluting with methanol/dichloromethane (1/15) to afford 2-(((2-amino-5-fluoropyridin-3-yl)methyl)amino)ethan-1-ol. hydrogen chloride (320 mg, 1.71 mmol, 91.1% yield) as a yellow solid. LC-MS: (ESI, m/z): 186.1 [M+H]⁺

Step 5: 5-(2-(((2-amino-5-fluoropyridin-3-yl)methyl)amino)ethoxy)-7-(6-(bis(4-methoxybenzyl)amino)-4-methyl-3-(trifluoromethyl)pyridin-2-yl)-6-chloroquinazolin-4(3H)-one

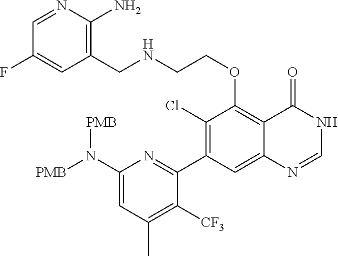

To a solution of 2-(((2-amino-5-fluoropyridin-3-yl)methyl)amino)ethan-1-ol. hydrogen chloride (271.9 mg, 1.4 mmol) in tetrahydrofuran (10 mL) was added sodium hydride (46.9 mg, 1.9 mmol, 60% purity) and stirred for 30 min at 0° C. Then 7-[6-[bis[(4-methoxyphenyl)methyl]amino]-4-methyl-3-(trifluoromethyl)-2-pyridyl]-6-chloro-5-fluoro-3H-quinazolin-4-one (300.0 mg, 0.49 mmol) was added and stirred at 65° C. for 5 hours. After completion, the reaction was quenched with 1N HCl and concentrated under vacuum. The residue was purified by flash chromatography on silica gel eluting with methanol/dichloromethane (1/10) to afford 5-(2-(((2-amino-5-fluoropyridin-3-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (160.0 mg, 0.2 mmol, 42.0% yield) as a yellow solid. LC-MS: (ESI, m/z): 778.2 [M+H]⁺

Step 6: 6-(4-((2-amino-5-fluoropyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

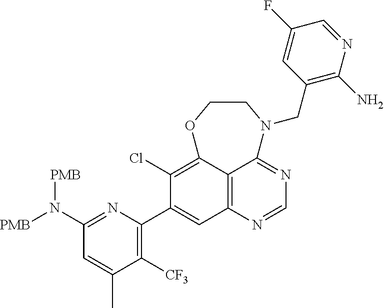

A solution of 5-(2-(((2-amino-5-fluoropyridin-3-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (140.0 mg, 0.1 mmol), benzotriazol-1-yloxytris(dimethylamino)-phosphonium hexafluorophosphate (112.3 mg, 0.2 mmol) and 1,8-diazabicyclo[5.4.0]undec-7-ene (82.0 mg, 0.5 mmol) in acetonitrile (5 mL) was stirred at 25° C. for 2 hours. After completion, the solvent was concentrated under vacuum. The resulting residue was purified by reverse phase chromatography (acetonitrile 66-70/0.1% TFA in water) to afford 6-(4-((2-amino-5-fluoropyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (50.0 mg, 0.06 mmol, 36.6% yield) as a yellow solid. LC-MS: (ESI, m/z): 760.2 [M+H]⁺

Step 7: 6-(4-((2-amino-5-fluoropyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

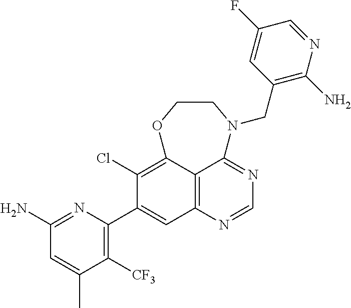 [SMILES: Cc1cc(N)nc(-c2cc3ncnc4c3c(c2Cl)OCCN4Cc2cc(F)cnc2N)c1C(F)(F)F]

A solution of 6-(4-((2-amino-5-fluoropyridin-3-yl)methyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (50.0 mg, 0.06 mmol) in trifluoroacetic acid (5 ml) was stirred at 50° C. for 8 hours. After completion, the solvent was concentrated under vacuum. The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min: Gradient: 31% B to 61% B in 7 min, 61% B: Wave Length: 254 nm; RT: 6.5 min) to afford 6-(4-((2-amino-5-fluoropyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine (7.6 mg, 0.01 mmol, 22.1% yield). LC-MS: (ESI, m/z): 520.2 [M+H]⁺

Example 31: ¹H NMR (400 MHz, DMSO-d₆, ppm) δ 8.41 (s, 1H), 7.86 (d, J=2.9 Hz, 1H), 7.30-7.23 (m, 1H), 7.21 (s, 1H), 6.76 (s, 2H), 6.46 (s, 1H), 5.94 (s, 2H), 4.83 (d, J=2.7 Hz, 2H), 4.81-4.63 (m, 2H), 3.94 (s, 2H), 2.37 (d, J=1.2 Hz, 3H).

Example 32: (R)-6-(8-chloro-4-(1-(5-((cyclopropylmethyl)amino)pyridin-3-yl)ethyl)-5,6-dihydro-4H-[1,4]oxazepino[5,6,7-de]quinazolin-9-yl)-4-methyl-5-(trifluoromethyl)pyridin-2-amine

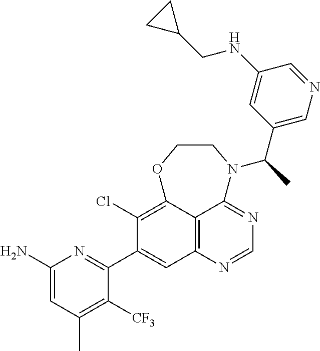 [SMILES: Cc1cc(N)nc(-c2cc3ncnc4c3c(c2Cl)OCCN4[C@H](C)c2cncc(NCC3CC3)c2)c1C(F)(F)F]

### Synthetic Route

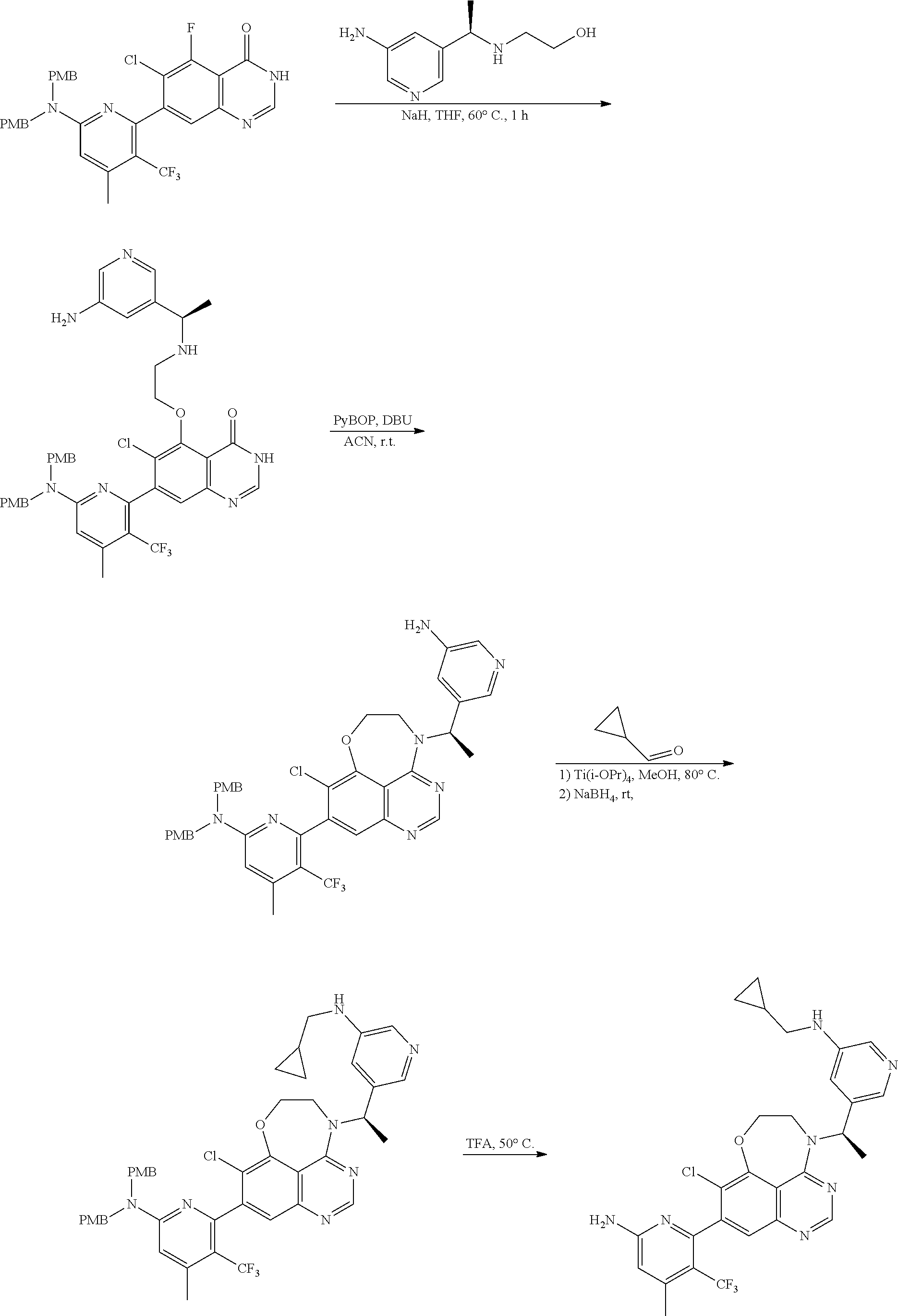

Step 1: (R)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one

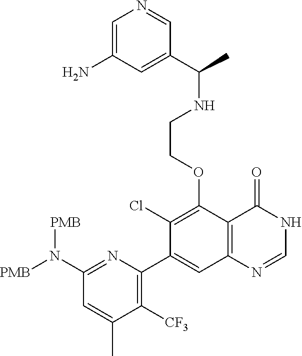

To a solution of 2-[[rac-(1R)-1-(5-amino-3-pyridyl)ethyl]amino]ethanol (325.2 mg, 1.7 mmol) in tetrahydrofuran (10 mL) was added sodium hydride (195.7 mg, 4.8 mmol, 60% purity) and stirred at 25° C. for 1 hour. Then 7-[6-[bis[(4-methoxyphenyl)methyl]amino]-4-methyl-3-(trifluoromethyl)-2-pyridyl]-6-chloro-5-fluoro-3H-quinazolin-4-one (1.00 g, 1.63 mmol) was added and stirred at 60° C. for 1 hour. After completion, the reaction was quenched with 1N HCl and concentrated under vacuum. The residue was purified by flash chromatography on silica gel eluting with dichloromethane/methanol (10/1) to afford (R)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (940 mg, 1.0 mmol, 63.3% yield) as a yellow solid. LC-MS: (ESI, m/z): 774.3 [M+H]⁺.

Step 2: (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

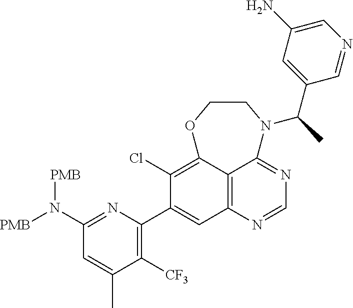

To a solution of (R)-5-(2-((1-(5-aminopyridin-3-yl)ethyl)amino)ethoxy)-7-(6-(bis(4-methoxybenzyl)amino)-4-methyl-3-(trifluoromethyl)pyridin-2-yl)-6-chloroquinazolin-4(3H)-one (920.0 mg, 1.1 mmol) and 1,8-diazabicyclo[5.4.0]undec-7-ene (542.7 mg, 3.5 mmol) in acetonitrile (10 mL) was added benzotriazol-1-yloxytris(dimethylamino)-phosphonium hexafluorophosphate (926.8 mg, 1.7 mmol), the mixture was stirred at 25° C. for 1 hour. After completion, the solvent was concentrated under vacuum. The resulting residue was purified by reverse phase chromatography (acetonitrile 76-80/0.1% TFA in water) to afford (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (720.0 mg, 0.8 mmol, 72.1% yield) as a yellow solid. LC-MS: (ESI, m/z): 756.3 [M+H]⁺

Step 3: (R)-6-(8-chloro-4-(1-(5-((cyclopropylmethyl)amino)pyridin-3-yl)ethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

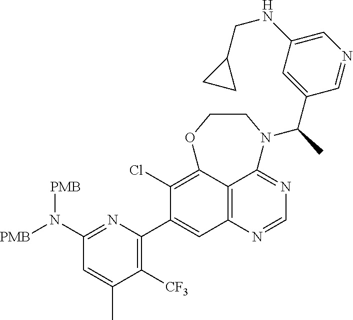

A mixture of (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (220.0 mg, 0.2 mmol), cyclopropanecarboxaldehyde (61.1 mg, 0.8 mmol) and titanium tetraisopropanolate (0.5 mL) in methyl alcohol (3 mL) was stirred for 3 hours at 80° C. Then sodium borohydride (22.1 mg, 0.5 mmol) was added and stirred at 25° C. for 1 hour. After completion, the reaction was quenched with water and concentrated under vacuum. The resulting residue was purified by reverse phase chromatography (acetonitrile 85-90/0.1% TFA in water) to afford (R)-6-(8-chloro-4-(1-(5-((cyclopropylmethyl)amino)pyridin-3-yl)ethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (129.0 mg, 0.1 mmol, 49.3% yield) as a yellow solid. LC-MS: (ESI, m/z): 810.3 [M+H]⁺

Step 4: (R)-6-(8-chloro-4-(1-(5-((cyclopropylmethyl)amino)pyridin-3-yl)ethyl)-5,6-dihydro-4H-[1,4]oxazepino[5,6,7-de]quinazolin-9-yl)-4-methyl-5-(trifluoromethyl)pyridin-2-amine

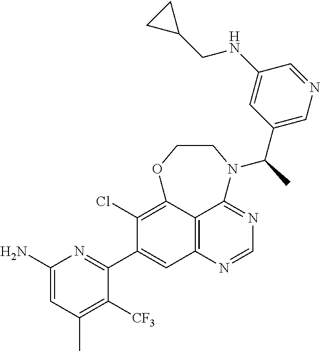 [SMILES: Cc1cc(N)nc(-c2cc3ncnc4c3c(c2Cl)OCCN4[C@H](C)c2cncc(NCC3CC3)c2)c1C(F)(F)F]

A solution of (R)-6-(8-chloro-4-(1-(5-((cyclopropylmethyl)amino)pyridin-3-yl)ethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (120.0 mg, 0.1 mmol) in trifluoroacetic acid (2 mL) was stirred at 50° C. for 8 hours. After completion, the solvent was concentrated under vacuum. The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 39% B to 69% B in 7 min; Wave Length: 254 nm; RT: 6.5 min) to afford (R)-6-(8-chloro-4-(1-(5-((cyclopropylmethyl)amino)pyridin-3-yl)ethyl)-5,6-dihydro-4H-[1,4]oxazepino[5,6,7-de]quinazolin-9-yl)-4-methyl-5-(trifluoromethyl)pyridin-2-amine (34.9 mg, 0.05 mmol, 39.4% yield). LC-MS: (ESI, m/z): 570.0 [M+H]

Example 32: ¹H NMR (300 MHz, Methanol-d4, ppm) δ 8.37 (d, J=1.1 Hz, 1H), 7.80-7.71 (m, 2H), 7.17 (s, 1H), 6.97 (s, 1H), 6.65 (s, 1H), 6.46 (s, 1H), 4.75-4.33 (m, 2H), 3.71-3.55 (m, 1H), 3.52-3.39 (m, 1H), 3.09-2.86 (m, 2H), 2.41 (d, J=2.5 Hz, 3H), 1.68 (d, J=6.9 Hz, 3H), 1.08-0.91 (m, 1H), 0.58-0.45 (m, 2H), 0.28-0.16 (m, 2H).

Example 33: (R)—N-(5-(1-(9-(6-amino-4-methyl-3-(trifluoromethyl)pyridin-2-yl)-8-chloro-5,6-dihydro-4H-[1,4]oxazepino[5,6,7-de]quinazolin-4-yl)ethyl)pyridin-3-yl)acetamide

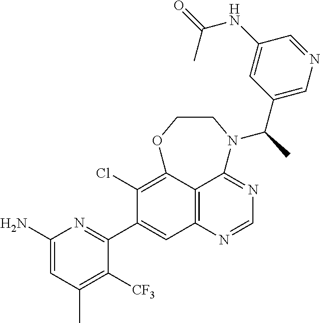 [SMILES: CC(=O)Nc1cncc([C@@H](C)N2CCOc3c(Cl)c(-c4nc(N)cc(C)c4C(F)(F)F)cc4ncnc2c34)c1]

### Synthetic Route

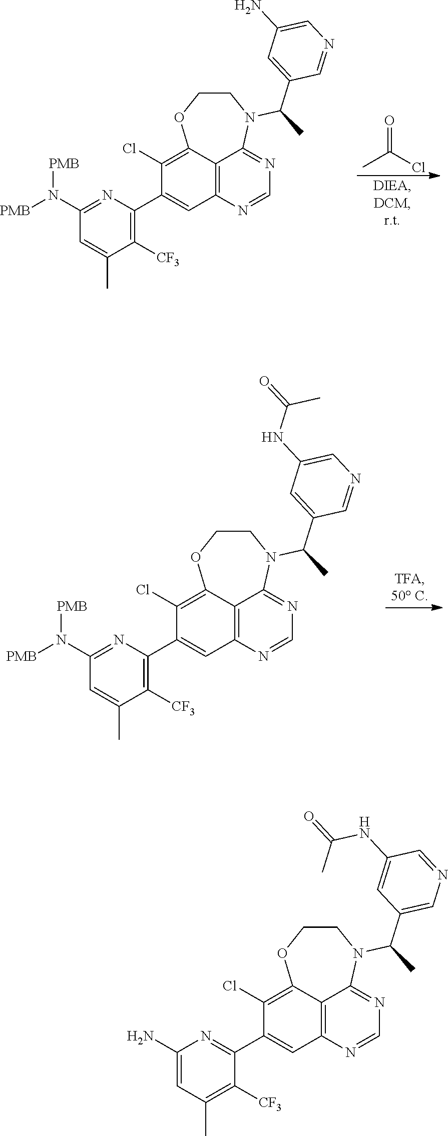

Step 1: (R)—N-(5-(1-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)ethyl)pyridin-3-yl)acetamide

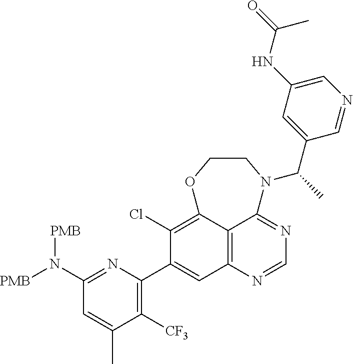

To a solution of (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (214.0 mg, 0.2 mmol) in dichloromethane (3 mL) was added triethylamine (85.9 mg, 0.8 mmol) and acetyl chloride (24.4 mg, 0.3 mmol), the mixture was stirred at 25° C. for 1 hour. After completion, the solvent was concentrated under vacuum. The resulting residue was purified by reverse phase chromatography (acetonitrile 62-70/0.1% TFA in water) to afford (R)—N-(5-(1-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)ethyl)pyridin-3-yl)acetamide (132.0 mg, 0.1 mmol, 56.7% yield) as a yellow solid. LC-MS: (ESI, m/z): 798.3 [M+H]⁺

Step 4: (R)—N-(5-(1-(9-(6-amino-4-methyl-3-(trifluoromethyl)pyridin-2-yl)-8-chloro-5,6-dihydro-4H-[1,4]oxazepino[5,6,7-de]quinazolin-4-yl)ethyl)pyridin-3-yl)acetamide

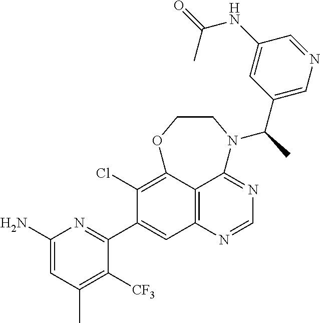 [SMILES: CC(=O)Nc1cncc([C@@H](C)N2CCOc3c(Cl)c(-c4nc(N)cc(C)c4C(F)(F)F)cc4ncnc2c34)c1]

A solution of (R)—N-(5-(1-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)ethyl)pyridin-3-yl)acetamide (125.0 mg, 0.1 mmol) in trifluoroacetic acid (357.1 mg, 3.1 mmol) was stirred at 50° C. for 8 hours. After completion, the solvent was concentrated under vacuum. The crude product was purified by Prep-HPLC with the following conditions: (Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 34% B to 50% B in 9 min, 50% B; Wave Length: 220/254 nm: RT: 7.73 min) to afford (R)—N-(5-(1-(9-(6-amino-4-methyl-3-(trifluoromethyl)pyridin-2-yl)-8-chloro-5,6-dihydro-4H-[1,4]oxazepino[5,6,7-de]quinazolin-4-yl)ethyl)pyridin-3-yl)acetamide (41.2 mg, 0.07 mmol, 46.3% yield). LC-MS: (ESI, m/z): 558.0 [M+H]⁺

Example 33: ¹H NMR (400 MHz, DMSO-d₆, ppm) δ 10.15-10.10 (m, 1H), 8.77-8.68 (m, 1H), 8.53-8.42 (m, 1H), 8.33-8.29 (m, 1H), 7.89 (d, J=17.3 Hz, 1H), 7.21-7.17 (m, 1H), 6.76 (d, J=16.9 Hz, 2H), 6.60 (s, 1H), 6.46-6.41 (m, 1H), 4.56-4.41 (m, 2H), 3.74-3.71 (m, 1H), 3.46-3.35 (m, 1H), 2.36-2.31 (m, 3H), 2.08-1.99 (m, 3H), 1.70-1.58 (m, 3H).

Example 34: (S)-6-(4-((2-aminopyridin-3-yl)methyl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

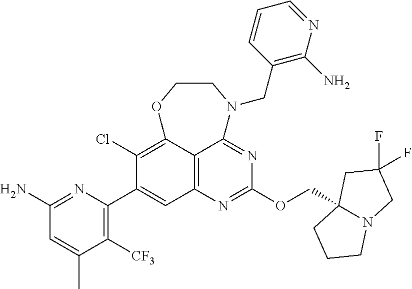 [SMILES: Cc1cc(N)nc(-c2cc3nc(OC[C@@]45CCCN4CC(F)(F)C5)nc4c3c(c2Cl)OCCN4Cc2cccnc2N)c1C(F)(F)F]

### Synthetic Route

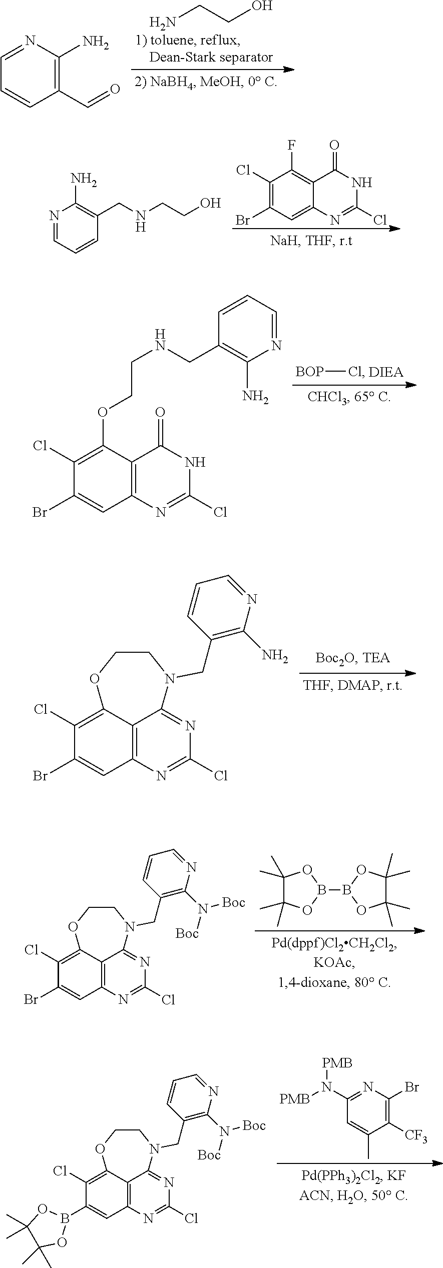

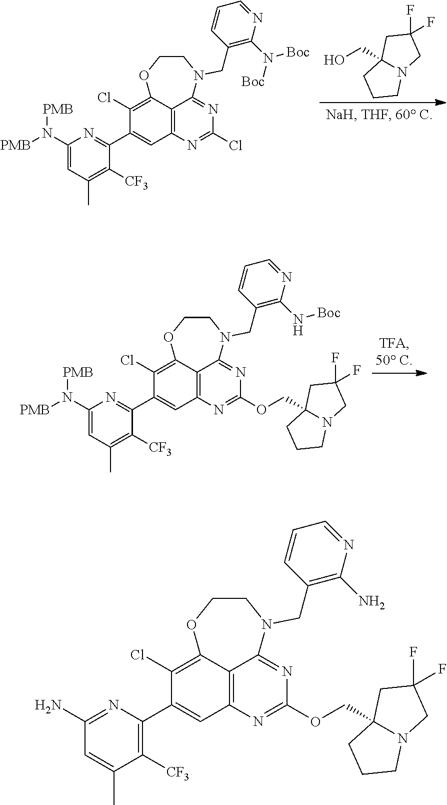

Step 1: 2-(((2-aminopyridin-3-yl)methyl)amino)ethan-1-ol

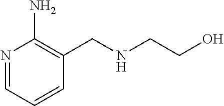 [SMILES: Nc1ncccc1CNCCO]

A solution of 2-aminonicotinaldehyde (10.00 g, 81.89 mmol) and 2-aminoethanol (5.00 g, 81.89 mmol) in toluene (200 mL) was stirred at 130° C. for 6 hours. The solvent was concentrated under vacuum. The mixture was dissolved in methyl alcohol (150 mL), sodium borohydride (6.19 g, 163.77 mmol) was added and stirred at 0° C. for 2 hours. The resulting solution was diluted with water and extracted with ethyl acetate. The organic layers were dried over anhydrous sodium sulfate and concentrated. The residue was purified by flash chromatography on silica gel eluting with methanol/dichloromethane (1/20) to afford 2-(((2-aminopyridin-3-yl)methyl)amino)ethan-1-ol (12.60 g, 74.60 mmol, 91.1% yield) as a yellow oil. LC-MS: (ESI, m/z): 168.1 [M+H]⁺

Step 2: 5-(2-(((2-aminopyridin-3-yl)methyl)amino)ethoxy)-7-bromo-2,6-dichloroquinazoline-4(3H)-one

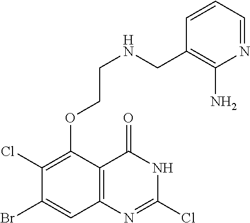 [SMILES: Nc1ncccc1CNCCOc1c(Cl)c(Br)cc2nc(Cl)[nH]c(=O)c12]

A solution of 2-(((2-aminopyridin-3-yl)methyl)amino)ethan-1-ol (900.0 mg, 5.3 mmol) and sodium hydride (538.2 mg, 13.4 mmol, 60% purity) in tetrahydrofuran (20 mL) was stirred at 0° C. for 1 hour. Then 7-bromo-2,6-dichloro-5-fluoro-3H-quinazolin-4-one (1.68 g, 5.38 mmol) was added and stirred at 25° C. for 1 hour. After completion, the reaction was quenched with 1N HCl and concentrated under vacuum to afford 5-(2-(((2-aminopyridin-3-yl)methyl)amino)ethoxy)-7-bromo-2,6-dichloroquinazoline-4(3H)-one (3.00 g crude) as a brown solid. LC-MS: (ESI, m/z): 458.0 [M+H]⁺

Step 3: 3-((9-bromo-2,8-dichloro-5,6-dihydro-4H-[1,4]oxazepino[5,6,7-de]quinazolin-4-yl)methyl)pyridin-2-amine

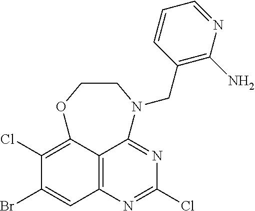 [SMILES: Nc1ncccc1CN1CCOc2c(Cl)c(Br)cc3nc(Cl)nc1c23]

A mixture of 5-(2-(((2-aminopyridin-3-yl)methyl)amino)ethoxy)-7-bromo-2,6-dichloroquinazoline-4(3H)-one (3.00 g crude), N,N-diisopropylethylamine (2.53 g, 19.60 mmol) and bis(2-oxo-3-oxazolidinyl)phosphinic chloride (2.66 g, 10.45 mmol) in chloroform (30 mL) was stirred at 65° C. for 1 hour. The resulting solution was diluted with dichloromethane, washed with brine, dried over anhydrous sodium sulfate and concentrated. The residue was purified by flash chromatography on silica gel eluting with ethyl acetate/dichloromethane (1/5) to afford 3-((9-bromo-2,8-dichloro-5,6-dihydro-4H-[1,4]oxazepino[5,6,7-de]quinazolin-4-yl)methyl)pyridin-2-amine (1.70 g, 3.66 mmol, 56% yield) as a yellow solid. LC-MS: (ESI, m/z): 440.0 [M+H]⁺

Step 4: tert-butyl N-[3-[7-bromo-3,8-dichloro-10-oxa-2,4,13-triazatricyclo[7.4.1.0^{5,14}]tetradeca-1,3,5(14),6,8-pentaen-13-yl)methyl]-2-pyridyl]-N-tert-butoxycarbonyl-carbamate

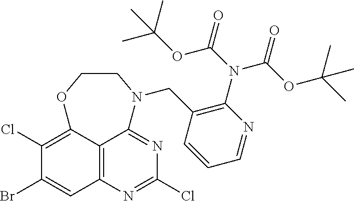 [SMILES: CC(C)(C)OC(=O)N(C(=O)OC(C)(C)C)c1ncccc1CN1CCOc2c(Cl)c(Br)cc3nc(Cl)nc1c23]

To a solution of 3-((9-bromo-2,8-dichloro-5,6-dihydro-4H-[1,4]oxazepino[5,6,7-de]quinazolin-4-yl)methyl)pyridin-2-amine (1.70 g, 3.85 mmol) and triethylamine (1.56 g, 15.42 mmol) in tetrahydrofuran (20 mL) was added 4-dimethylaminopyridine (47.0 mg, 0.3 mmol) and di-tert-butyl dicarbonate (2.94 g, 13.49 mmol), the mixture was stirred at 25° C. for 16 hours. The resulting solution was diluted with ethyl acetate, washed with brine, dried over anhydrous sodium sulfate and concentrated. The residue was purified by flash chromatography on silica gel eluting with ethyl acetate/dichloromethane (1/10) to afford tert-butyl N-[3-[(7-bromo-3,8-dichloro-10-oxa-2,4,13-triazatricyclo[7.4.1.0^{5,14}]tetradeca-1,3,5(14),6,8-pentaen-13-yl)methyl]-2-pyridyl]-N-tert-butoxycarbonyl-carbamate (1.40 g, 2.11 mmol, 54.9% yield) as a yellow solid. LC-MS: (ESI, m/z): 640.1 [M+H]⁺

Step 5: tert-butyl N-tert-butoxycarbonyl-N-[3-[[3,8-dichloro-7-(4,4,5,5-tetramethyl-1,3,2-dioxaborolan-2-yl)-10-oxa-2,4,13-triazatricyclo[7.4.1.0^{5,14}]tetradeca-1,3,5(14),6,8-pentaen-13-yl]methyl]-2-pyridyl]carbamate

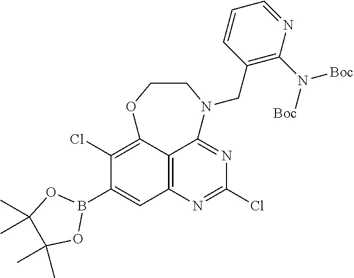 [SMILES: CC(C)(C)OC(=O)N(C(=O)OC(C)(C)C)c1ncccc1CN1CCOc2c(Cl)c(B3OC(C)(C)C(C)(C)O3)cc3nc(Cl)nc1c23]

Under nitrogen, a mixture of tert-butyl N-[3-[(7-bromo-3,8-dichloro-10-oxa-2,4,13-triazatricyclo[7.4.1.0^{5,14}]tetradeca-1,3,5(14),6,8-pentaen-13-yl)methyl]-2-pyridyl]-N-tert-butoxycarbonyl-carbamate (950.0 mg, 1.4 mmol), bis(pinacolato)diboron (752.3 mg, 2.9 mmol), potassium acetate (290.7 mg, 2.9 mmol) and [1,1′-bis(diphenylphosphino)ferrocene]dichloropalladium(II) (109.8 mg, 0.1 mmol) in 1,4-dioxane (12 mL) was stirred at 80° C. for 2 hours. The reaction mixture was filtered and washed with ethyl acetate. The filtrate was concentrated under reduced pressure. The crude (1.6 g crude) was used directly without further purification. LC-MS: (ESI, m/z): 688.2 [M+H]⁺

Step 6: tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.0^{5,14}]tetradeca-1,3,5(14),6,8-pentaen-13-yl]methyl]-2-pyridyl]-N-tert-butoxycarbonyl-carbamate

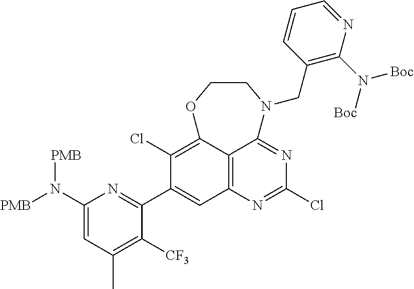

Under nitrogen, a solution of tert-butyl N-tert-butoxycarbonyl-N-[3-[[3,8-dichloro-7-(4,4,5,5-tetramethyl-1,3,2-dioxaborolan-2-yl)-10-oxa-2,4,13-triazatricyclo[7.4.1.0⁵¹4]tetradeca-1,3,5(14),6,8-pentaen-13-yl]methyl]-2-pyridyl]carbamate (1.60 g crude), 6-bromo-N,N-bis[(4-methoxyphenyl)methyl]-4-methyl-5-(trifluoromethyl)pyridin-2-amine (460.5 mg, 0.9 mmol), potassium fluoride (270.0 mg, 4.6 mmol) and bis(triphenylphosphine)palladium(II) chloride (163.1 mg, 0.2 mmol) in acetonitrile (10 mL) and water (2 mL) was stirred at 50° C. for 1 hour. The resulting solution was diluted with water and extracted with ethyl acetate. The organic layers were washed with brine, dried over anhydrous Sodium sulfate and concentrated. The residue was purified by flash chromatography on silica gel eluting with ethyl acetate/dichloromethane (1/5) to afford tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.0^{5,14}]tetradeca-1,3,5(14),6,8-pentaen-13-yl]methyl]-2-pyridyl]-N-tert-butoxycarbonyl-carbamate (1.20 g, 1.10 mmol, 47.6% yield) as a yellow solid. LC-MS: (ESI, m/z): 976.3 [M+H]⁺

Step 7: tert-butyl (S)-(3-((9-(6-(bis(4-methoxybenzyl)amino)-4-methyl-3-(trifluoromethyl)pyridin-2-yl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-4-yl)methyl)pyridin-2-yl)carbamate

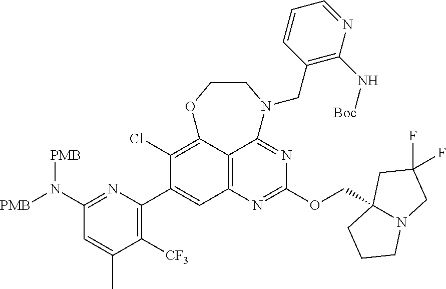

A solution of (S)-(2,2-difluorotetrahydro-1H-pyrrolizin-7a(5H)-yl)methanol (108.8 mg, 0.61 mmol) and sodium hydride (49.1 mg, 1.2 mmol, 60% purity) in tetrahydrofuran (1 mL) was stirred at 25° C. for 1 hour. Then tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.0^{5,14}]tetradeca-1,3,5(14),6,8-pentaen-13-yl]methyl]-2-pyridyl]-N-tert-butoxycarbonyl-carbamate (120.0 mg, 0.1 mmol) was added and stirred at 60° C. for 3 hours. After completion, the reaction was quenched with 1N HCl and concentrated under vacuum. The residue was purified by flash chromatography on silica gel eluting with ethyl acetate/dichloromethane (1/1) to afford tert-butyl (S)-(3-((9-(6-(bis(4-methoxybenzyl)amino)-4-methyl-3-(trifluoromethyl)pyridin-2-yl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-4-yl)methyl)pyridin-2-yl)carbamate (101.0 mg, 0.08 mmol, 69.9% yield) as a yellow solid. LC-MS: (ESI, m/z): 1017.4 [M+H]⁺

Step 8: (S)-6-(4-((2-aminopyridin-3-yl)methyl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

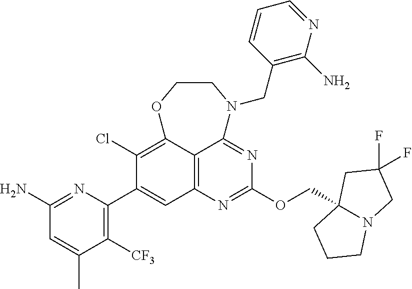 [SMILES: Cc1cc(N)nc(-c2cc3nc(OC[C@@]45CCCN4CC(F)(F)C5)nc4c3c(c2Cl)OCCN4Cc2cccnc2N)c1C(F)(F)F]

A solution of tert-butyl (S)-(3-((9-(6-(bis(4-methoxybenzyl)amino)-4-methyl-3-(trifluoromethyl)pyridin-2-yl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-4-yl)methyl)pyridin-2-yl)carbamate (95.0 mg, 0.09 mmol) in trifluoroacetic acid (2 mL) was stirred at 50° C. for 6 hours. After completion, the solvent was concentrated under vacuum. The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 39% B to 69% B in 7 min, 69% B: Wave Length: 245 nm; RT: 6.5 min) to afford (S)-6-(4-((2-aminopyridin-3-yl)methyl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (20.2 mg, 0.02 mmol, 31.7% yield). LC-MS: (ESI, m/z): 677.3 [M+H]⁺

Example 34: ¹H NMR (400 MHz, DMSO-d₆, ppm) δ 7.89 (d, J=4.9 Hz, 1H), 7.29 (d, J=7.3 Hz, 1H), 6.96 (d, J=1.6 Hz, 1H), 6.74 (s, 2H), 6.56-6.48 (m, 1H), 6.44 (s, 1H), 6.04 (s, 2H), 4.88-4.78 (m, 2H), 4.62-4.59 (m, 2H), 4.03-3.95 (m, 2H), 3.91-3.86 (m, 2H), 3.32-3.22 (m, 1H), 3.09-2.81 (m, 2H), 2.50-2.25 (m, 6H), 1.96-1.85 (m, 1H), 1.83-1.81 (m, 1H), 1.76-1.66 (m, 2H).

Example 35: (S)-6-(4-((2-aminopyridin-3-yl)methyl)-8-chloro-2-((dihydro-1H,3H-spiro[pyrrolizine-2,2′-[1,3]dioxolan]-7a(5H)-yl)methoxy)-5,6-dihydro-4H-[1,4]oxazepino[5,6,7-de]quinazolin-9-yl)-4-methyl-5-(trifluoromethyl)pyridin-2-amine

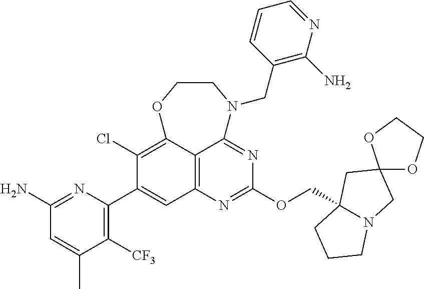 [SMILES: Cc1cc(N)nc(-c2cc3nc(OC[C@@]45CCCN4CC4(C5)OCCO4)nc4c3c(c2Cl)OCCN4Cc2cccnc2N)c1C(F)(F)F]

### Synthetic Route

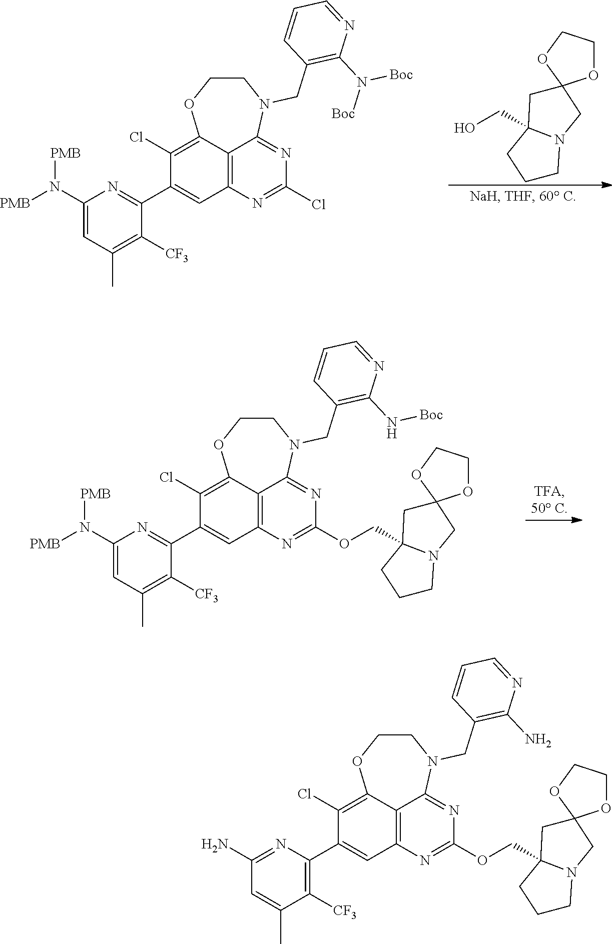

Step 1: tert-butyl (S)-(3-((9-(6-(bis(4-methoxybenzyl)amino)-4-methyl-3-(trifluoromethyl)pyridin-2-yl)-8-chloro-2-((dihydro-1H,3H-spiro[pyrrolizine-2,2′-[1,3]dioxolan]-7a(5H)-yl)methoxy)-5,6-dihydro-4H-[1,4]oxazepino[5,6,7-de]quinazolin-4-yl)methyl)pyridin-2-yl)carbamate

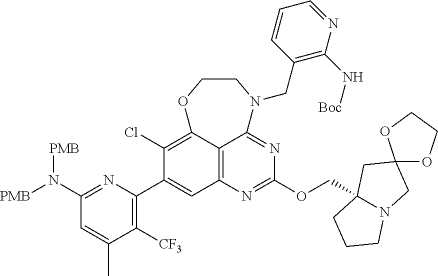

A solution of (S)-(dihydro-1H,3H-spiro[pyrrolizine-2,2′-[1,3]dioxolan]-7a(5H)-yl)methanol (203.9 mg, 1.0 mmol) and sodium hydride (81.8 mg, 2.0 mmol, 60% purity) in tetrahydrofuran (5 mL) was stirred at 25° C. for 1 hour. Then tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.0^{5,14}]tetradeca-1,3,5(14),6,8-pentaen-13-yl]methyl]-2-pyridyl]-N-tert-butoxycarbonyl-carbamate (200.0 mg, 0.2 mmol) was added and stirred at 60° C. for 1 hour. After completion, the reaction was quenched with 1N HCl and concentrated under vacuum. The residue was purified by flash chromatography on silica gel eluting with dichloromethane/methanol (15/1) to afford tert-butyl (S)-(3-((9-(6-(bis(4-methoxybenzyl)amino)-4-methyl-3-(trifluoromethyl)pyridin-2-yl)-8-chloro-2-((dihydro-1H,3H-spiro[pyrrolizine-2,2′-[1,3]dioxolan]-7a(5H)-yl)methoxy)-5,6-dihydro-4H-[1,4]oxazepino[5,6,7-de]quinazolin-4-yl)methyl)pyridin-2-yl)carbamate (159.0 mg, 0.1 mmol, 71% yield) as a yellow solid. LC-MS: (ESI, m/z): 1039.4 [M+H]⁺

Step 2: (S)-6-(4-((2-aminopyridin-3-yl)methyl)-8-chloro-2-((dihydro-1H,3H-spiro[pyrrolizine-2,2′-[1,3]dioxolan]-7a(5H)-yl)methoxy)-5,6-dihydro-4H-[1,4]oxazepino[5,6,7-de]quinazolin-9-yl)-4-methyl-5-(trifluoromethyl)pyridin-2-amine

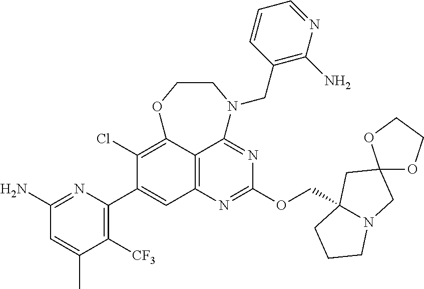 [SMILES: Cc1cc(N)nc(-c2cc3nc(OC[C@@]45CCCN4CC4(C5)OCCO4)nc4c3c(c2Cl)OCCN4Cc2cccnc2N)c1C(F)(F)F]

A solution of tert-butyl (S)-(3-((9-(6-(bis(4-methoxybenzyl)amino)-4-methyl-3-(trifluoromethyl)pyridin-2-yl)-8-chloro-2-((dihydro-1H,3H-spiro[pyrrolizine-2,2′-[1,3]dioxolan]-7a(5H)-yl)methoxy)-5,6-dihydro-4H-[1,4]oxazepino[5,6,7-de]quinazolin-4-yl)methyl)pyridin-2-yl)carbamate (150.0 mg, 0.1 mmol) in trifluoroacetic acid (5 ml) was stirred at 50° C. for 6 hours. The solvent was concentrated under vacuum. The crude product was purified by Prep-HPLC with the following conditions: (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 32% B to 62% B in 7 min, 62% B; Wave Length: 254 nm; RT: 6.5 min) to afford (S)-6-(4-((2-aminopyridin-3-yl)methyl)-8-chloro-2-((dihydro-1H,3H-spiro[pyrrolizine-2,2′-[1,3]dioxolan]-7a(5H)-yl)methoxy)-5,6-dihydro-4H-[1,4]oxazepino[5,6,7-de]quinazolin-9-yl)-4-methyl-5-(trifluoromethyl)pyridin-2-amine (15.8 mg, 0.02 mmol, 15.6% yield). LC-MS: (ESI, m/z): 699.1 [M+H]⁺

Example 35: ¹H NMR (400 MHz, DMSO-d₆, ppm) δ 7.91-7.90 (m, 1H), 7.33-7.31 (m, 1H), 6.97 (s, 1H), 6.74 (s, 2H), 6.56-6.52 (m, 1H), 6.45 (s, 1H), 6.04 (s, 2H), 4.92-4.80 (m, 2H), 4.66-4.60 (m, 2H), 4.12-3.95 (m, 2H), 3.83-3.68 (m, 6H), 3.02-2.98 (m, 2H), 2.74-2.71 (m, 2H), 2.36 (d, J=2.3 Hz, 3H), 2.08 (d, J=13.6 Hz, 1H), 1.88 (d, J=13.5 Hz, 3H), 1.69 (s, 2H).

Example 36: 6-(2-((2-oxabicyclo[2.1.1]hexan-4-yl)methoxy)-4-((2-aminopyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

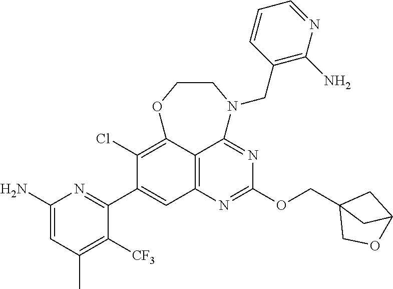 [SMILES: Cc1cc(N)nc(-c2cc3nc(OCC45COC(C4)C5)nc4c3c(c2Cl)OCCN4Cc2cccnc2N)c1C(F)(F)F]

### Synthetic Route

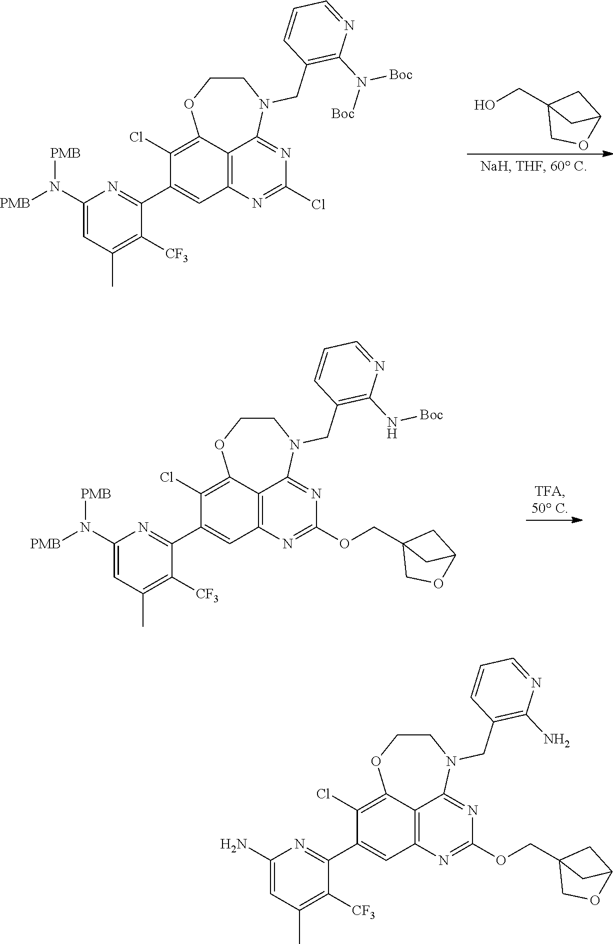

Step 1: tert-butyl (3-((2-((2-oxabicyclo[2.1.1]hexan-4-yl)methoxy)-9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridin-2-yl)carbamate

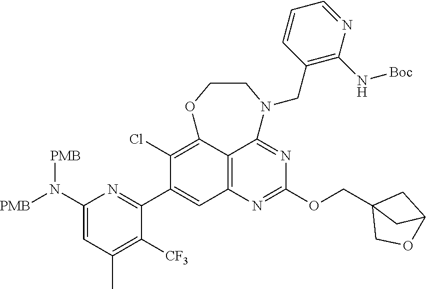

A solution of 2-oxabicyclo[2.1.1]hexan-4-ylmethanol (28.7 mg, 0.2 mmol) and sodium hydride (24 mg, 0.6 mmol, 60% purity) in tetrahydrofuran (2 mL) was stirred at 25° C. for 1 hour. Then tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.0^{5,14}]tetradeca-1,3,5(14),6,8-pentaen-13-yl]methyl]-2-pyridyl]-N-tert-butoxycarbonyl-carbamate (205.0 mg, 0.2 mmol) was added and stirred at 60° C. for 6 hours. After completion, the reaction was quenched with 1N HCl and concentrated under vacuum. The residue was purified by flash chromatography on silica gel eluting with ethyl acetate/dichloromethane (4/1) to afford tert-butyl (3-((2-((2-oxabicyclo[2.1.1]hexan-4-yl)methoxy)-9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridin-2-yl)carbamate (110.0 mg, 0.1 mmol, 52.2% yield) as a yellow solid. LC-MS: (ESI, m/z): 954.3 [M+H]⁺

Step 2: 6-(2-((2-oxabicyclo[2.1.1]hexan-4-yl)methoxy)-4-((2-aminopyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

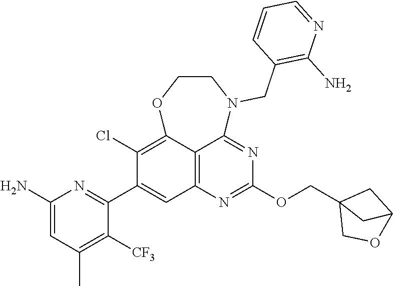 [SMILES: Cc1cc(N)nc(-c2cc3nc(OCC45COC(C4)C5)nc4c3c(c2Cl)OCCN4Cc2cccnc2N)c1C(F)(F)F]

A solution of tert-butyl (3-((2-((2-oxabicyclo[2.1.1]hexan-4-yl)methoxy)-9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridin-2-yl)carbamate (105.0 mg, 0.1 mmol) in trifluoroacetic acid (5 ml) was stirred at 50° C. for 8 hours. After completion, the solvent was concentrated under vacuum. The crude product was purified by Prep-HPLC with the following conditions: (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 23% B to 50% B in 9 min, 50% B; Wave Length: 254/220 nm; RT: 9.67 min) to afford 6-(2-((2-oxabicyclo[2.1.1]hexan-4-yl)methoxy)-4-((2-aminopyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine (29.0 mg, 0.04 mmol, 42.8% yield). LC-MS: (ESI, m/z): 614.2 [M+H]⁺

Example 36: ¹H NMR (300 MHz, DMSO-d₆, ppm) δ 7.94-7.86 (m, 1H), 7.33-7.24 (m, 1H), 6.97 (s, 1H), 6.74 (s, 2H), 6.58-6.48 (m, 1H), 6.45 (s, 1H), 6.04 (s, 2H), 4.84 (s, 2H), 4.71-4.42 (m, 5H), 3.98-3.80 (m, 2H), 3.51 (s, 2H), 2.36 (d, J=2.3 Hz, 3H), 1.77-1.67 (m, 2H), 1.49-1.35 (m, 2H).

Example 37: 7-(9-(6-amino-4-methyl-3-(trifluoromethyl)pyridin-2-yl)-8-chloro-5,6-dihydro-4H-[1,4]oxazepino[5,6,7-de]quinazolin-4-yl)-6,7-dihydro-5H-cyclopenta[c]pyridin-4-amine

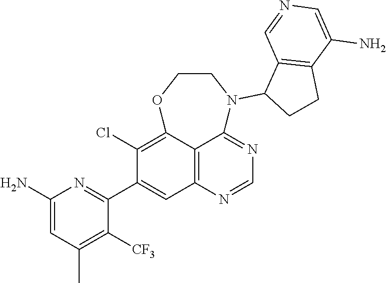 [SMILES: Cc1cc(N)nc(-c2cc3ncnc4c3c(c2Cl)OCCN4C2CCc3c(N)cncc32)c1C(F)(F)F]

### Synthetic Route

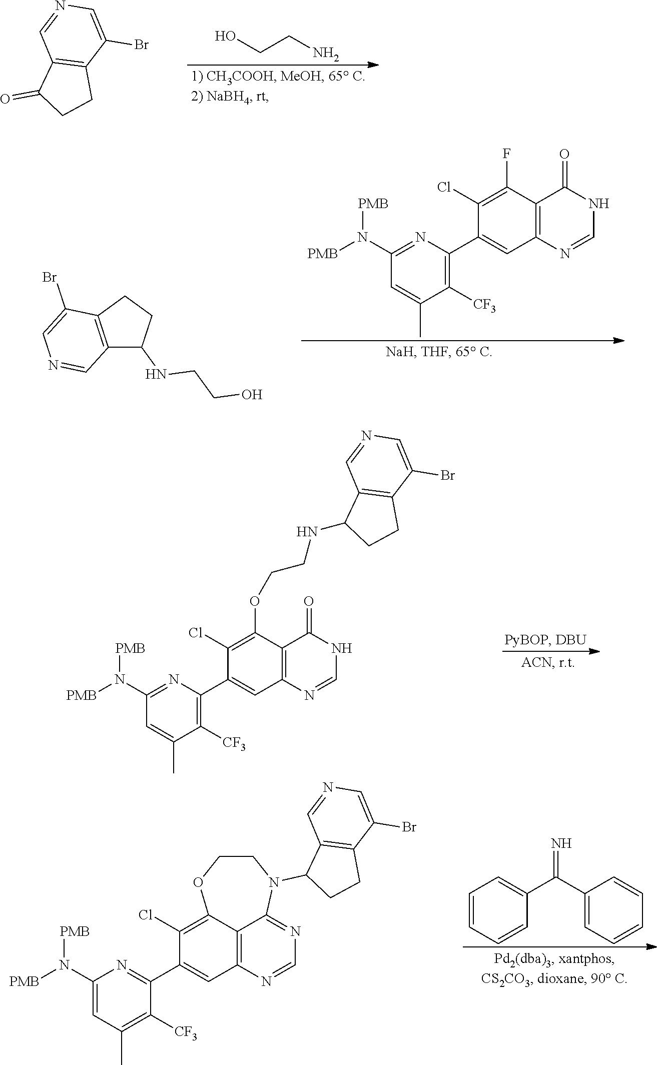

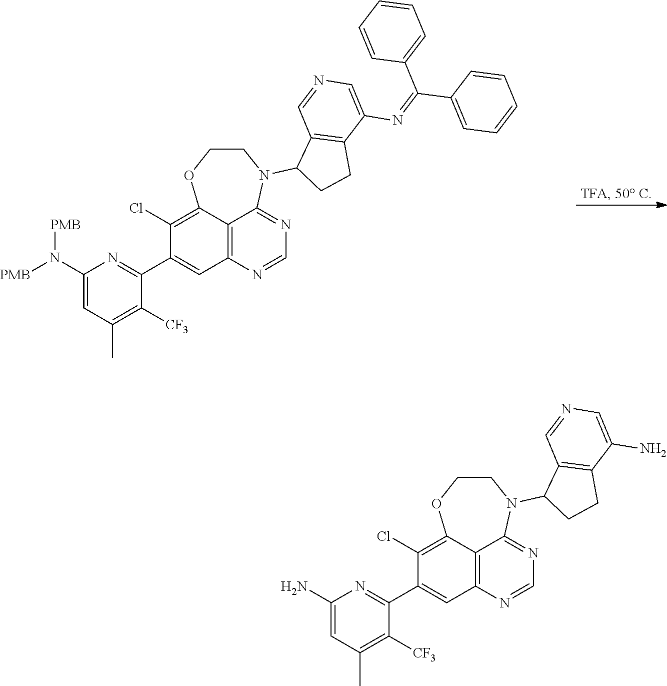

Step 1: 2-((4-bromo-6,7-dihydro-5H-cyclopenta[c]pyridin-7-yl)amino)ethan-1-ol

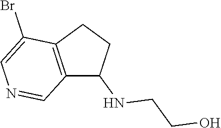 [SMILES: OCCNC1CCc2c(Br)cncc21]

A solution of 4-bromo-5,6-dihydrocyclopenta[c]pyridin-7-one (1.00 g, 4.72 mmol) and 2-aminoethanol (576.1 mg, 9.4 mmol), acetic acid (28.3 mg, 0.4 mmol) in methyl alcohol (10 mL) was stirred at 80° C. for 3 hours. Then sodium borohydride (358.4 mg, 9.4 mmol) was added and stirred at 25° C. for 1 hour. After completion, the reaction was quenched with water and the solvent was concentrated under vacuum. The residue was purified by flash chromatography on silica gel eluting with dichloromethane/methanol (20/1) to afford 2-((4-bromo-6,7-dihydro-5H-cyclopenta[c]pyridin-7-yl)amino)ethan-1-ol (946.0 mg, 3.5 mmol, 74.9% yield) as a brown oil. LC-MS: (ESI, m/z): 257.0 [M+H]⁺

Step 2: 7-(6-(bis(4-methoxybenzyl)amino)-4-methyl-3-(trifluoromethyl)pyridin-2-yl)-5-(2-((4-bromo-6,7-dihydro-5H-cyclopenta[c]pyridin-7-yl)amino)ethoxy)-6-chloroquinazolin-4(3H)-one

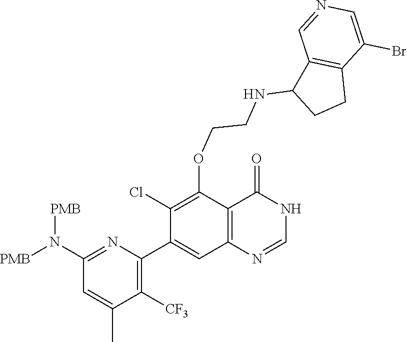

A solution of 2-((4-bromo-6,7-dihydro-5H-cyclopenta[c]pyridin-7-yl)amino)ethan-1-ol (251.6 mg, 0.9 mmol) and sodium hydride (97.8 mg, 2.4 mmol, 60% purity) in tetrahydrofuran (6 mL) was stirred at 25° C. for 1 hour. Then 7-[6-[bis[(4-methoxyphenyl)methyl]amino]-4-methyl-3-(trifluoromethyl)-2-pyridyl]-6-chloro-5-fluoro-3H-quinazolin-4-one (500.0 mg, 0.8 mmol) was added and stirred at 60° C. for 1 hour. After completion, the reaction was quenched with 1N HCl and concentrated under vacuum. The residue was purified by flash chromatography on silica gel eluting with methanol/dichloromethane (1/6) to afford 7-(6-(bis(4-methoxybenzyl)amino)-4-methyl-3-(trifluoromethyl)pyridin-2-yl)-5-(2-((4-bromo-6,7-dihydro-5H-cyclopenta[c]pyridin-7-yl)amino)ethoxy)-6-chloroquinazolin-4(3H)-one (534.0 mg, 0.5 mmol, 70.8% yield) as a brown solid. LC-MS: (ESI, m/z): 849.2 [M+H]⁺

Step 3: 6-(4-(4-bromo-6,7-dihydro-5H-cyclopenta[c]pyridin-7-yl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

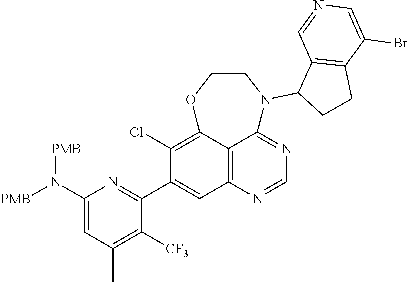

A solution of 7-(6-(bis(4-methoxybenzyl)amino)-4-methyl-3-(trifluoromethyl)pyridin-2-yl)-5-(2-((4-bromo-6,7-dihydro-5H-cyclopenta[c]pyridin-7-yl)amino)ethoxy)-6-chloroquinazolin-4(3H)-one (524.0 mg, 0.6 mmol), 1,8-diazabicyclo[5.4.0]undec-7-ene (281.5 mg, 1.8 mmol) and benzotriazol-1-yloxytris(dimethylamino)-phosphonium hexafluorophosphate (480.7 mg, 0.9 mmol) in acetonitrile (6 mL) was stirred at 25° C. for 1 hour. After completion, the solvent was concentrated under vacuum. The residue was purified by flash chromatography on silica gel eluting with methanol/dichloromethane (1/20) to afford 6-(4-(4-bromo-6,7-dihydro-5H-cyclopenta[c]pyridin-7-yl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (417.0 mg, 0.4 mmol, 76.4% yield) as a yellow solid. LC-MS: (ESI, m/z): 831.2 [M+H]⁺

Step 4: 6-(8-chloro-4-(4-((diphenylmethylene)amino)-6,7-dihydro-5H-cyclopenta[c]pyridin-7-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

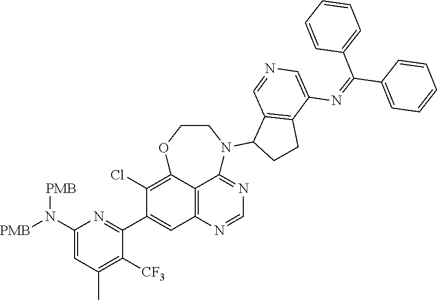

Under nitrogen, a solution of 6-(4-(4-bromo-6,7-dihydro-5H-cyclopenta[c]pyridin-7-yl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.0 mg, 0.4 mmol), benzophenone imine (261.3 mg, 1.4 mmol), tris(dibenzylideneacetone)dipalladium (43.9 mg, 0.05 mmol), 4,5-bis(diphenylphosphino)-9,9-dimethylxanthene (55.6 mg, 0.1 mmol) and cesium carbonate (313.4 mg, 0.9 mmol) in 1,4-dioxane (5 mL) was stirred at 90° C. for 16 hours. After completion, the solvent was concentrated under vacuum. The residue was purified by flash chromatography on silica gel eluting with dichloromethane/methanol (6/1) to afford 6-(8-chloro-4-(4-((diphenylmethylene)amino)-6,7-dihydro-5H-cyclopenta[c]pyridin-7-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.0 mg, 0.4 mmol, 88.3% yield) as a yellow solid. LC-MS: (ESI, m/z): 932.3 [M+H]⁺

Step 5: 7-(9-(6-amino-4-methyl-3-(trifluoromethyl)pyridin-2-yl)-8-chloro-5,6-dihydro-4H-[1,4]oxazepino[5,6,7-de]quinazolin-4-yl)-6,7-dihydro-5H-cyclopenta[c]pyridin-4-amine

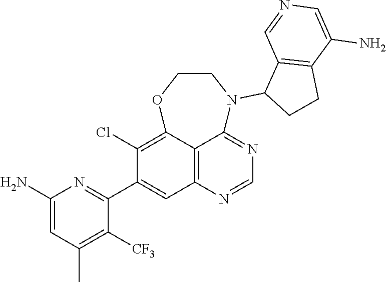 [SMILES: Cc1cc(N)nc(-c2cc3ncnc4c3c(c2Cl)OCCN4C2CCc3c(N)cncc32)c1C(F)(F)F]

A solution of 6-(8-chloro-4-(4-((diphenylmethylene)amino)-6,7-dihydro-5H-cyclopenta[c]pyridin-7-yl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.2 mmol) in trifluoroacetic acid (10 ml) was stirred at 50° C. for 8 hours. After completion, the solvent was concentrated under vacuum. The crude product was purified by Prep-HPLC with the following conditions: (Column: XBridge Prep OBD C18 Column, 19*250 mm, 5 μm; Mobile Phase A: Water (10 mmol/L NH₄HCOs), Mobile Phase B: ACN; Flow rate: 60 mL/min; Gradient: 18% B to 38% B in 10 min, 38% B; Wave Length: 254/220 nm) to afford 7-(9-(6-amino-4-methyl-3-(trifluoromethyl)pyridin-2-yl)-8-chloro-5,6-dihydro-4H-[1,4]oxazepino[5,6,7-de]quinazolin-4-yl)-6,7-dihydro-5H-cyclopenta[c]pyridin-4-amine (18.0 mg, 0.03 mmol, 15.8% yield). LC-MS: (ESI, m/z): 528.1 [M+H]⁺

Example 37: ¹H NMR (300 MHz, DMSO-d₆, ppm) δ 8.50 (s, 1H), 7.84 (s, 1H), 7.72 (d, J=12.7 Hz, 1H), 7.22 (s, 1H), 6.89-6.75 (m, 3H), 6.46 (s, 1H), 5.33 (s, 2H), 4.65-4.45 (m, 2H), 3.58-3.41 (m, 2H), 2.98-2.82 (m, 1H), 2.77-2.56 (m, 2H), 2.37 (d, J=2.5 Hz, 3H), 2.10-1.94 (m, 1H).

Example 38: (R)-6-(8-chloro-4-(1-(5-((pyridin-3-ylmethyl)amino)pyridin-3-yl)ethyl)-5,6-dihydro-4H-[1,4]oxazepino[5,6,7-de]quinazolin-9-yl)-4-methyl-5-(trifluoromethyl)pyridin-2-amine

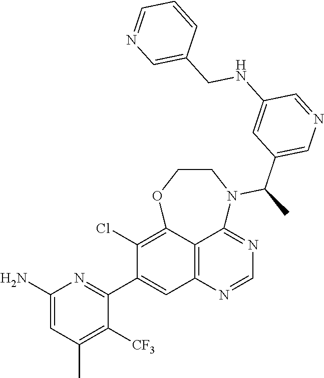 [SMILES: Cc1cc(N)nc(-c2cc3ncnc4c3c(c2Cl)OCCN4[C@H](C)c2cncc(NCc3cccnc3)c2)c1C(F)(F)F]

### Synthetic Route

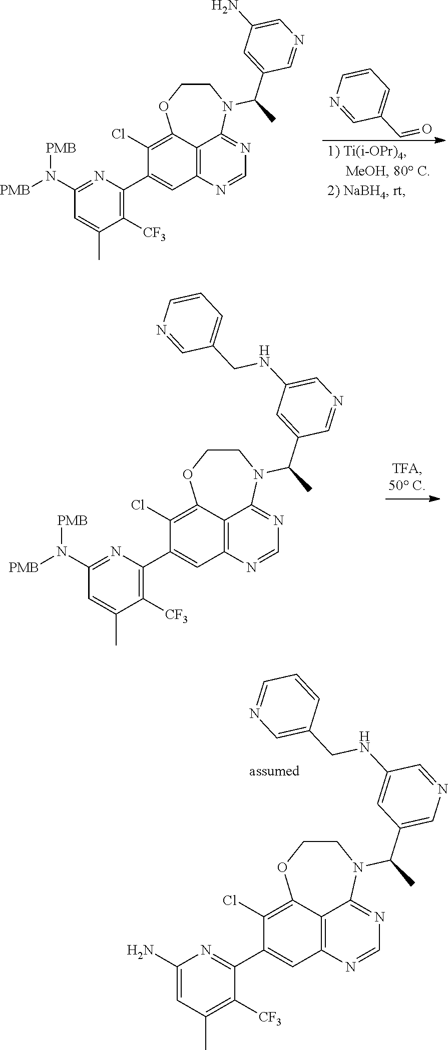

Step 1: (R)-6-(8-chloro-4-(1-(5-((pyridin-3-ylmethyl)amino)pyridin-3-yl)ethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

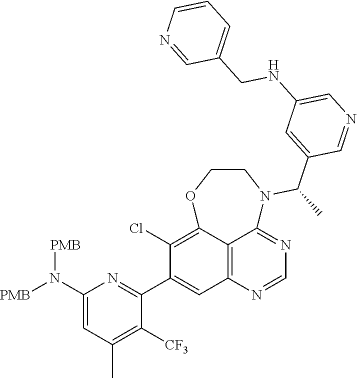

A solution of (R)-6-(4-(1-(5-aminopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (240.0 mg, 0.32 mmol), nicotinaldehyde (67.9 mg, 0.6 mmol) and titanium tetraisopropanolate (0.4 mL) in methyl alcohol (4 mL) was stirred for 3 hours at 80° C. Then sodium borohydride (24.1 mg, 0.6 mmol) was added and stirred at 25° C. for 1 hour. After completion, the reaction was quenched with water and the solvent was concentrated under vacuum. The resulting residue was purified by reverse phase chromatography (acetonitrile 70-75/0.1% TFA in water) to afford (R)-6-(8-chloro-4-(1-(5-((pyridin-3-ylmethyl)amino)pyridin-3-yl)ethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (81.0 mg, 0.07 mmol, 23.5% yield) as a yellow solid. LC-MS: (ESI, m/z): 847.3 [M+H]⁺

Step 2: (R)-6-(8-chloro-4-(1-(5-((pyridin-3-ylmethyl)amino)pyridin-3-yl)ethyl)-5,6-dihydro-4H-[1,4]oxazepino[5,6,7-de]quinazolin-9-yl)-4-methyl-5-(trifluoromethyl)pyridin-2-amine

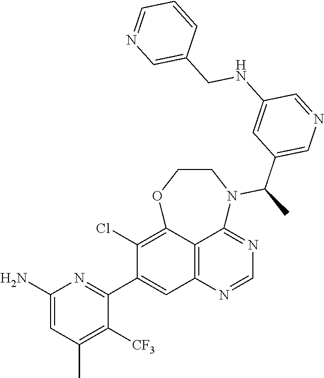 [SMILES: Cc1cc(N)nc(-c2cc3ncnc4c3c(c2Cl)OCCN4[C@H](C)c2cncc(NCc3cccnc3)c2)c1C(F)(F)F]

A solution of (R)-6-(8-chloro-4-(1-(5-((pyridin-3-ylmethyl)amino)pyridin-3-yl)ethyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (80.0 mg, 0.09 mmol) in trifluoroacetic acid (5 ml) was stirred at 50° C. for 8 hours. After completion, the solvent was concentrated under vacuum. The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 30% B to 60% B in 7 min: Wave Length: 254 nm; RT: 6.5 min) to afford (R)-6-(8-chloro-4-(1-(5-((pyridin-3-ylmethyl)amino)pyridin-3-yl)ethyl)-5,6-dihydro-4H-[1,4]oxazepino[5,6,7-de]quinazolin-9-yl)-4-methyl-5-(trifluoromethyl)pyridin-2-amine (26.8 mg, 0.04 mmol, 43.9% yield). LC-MS: (ESI, m/z): 607.0 [M+H]⁺

Example 38: ¹H NMR (400 MHz, DMSO-d₆, ppm) δ 8.56-8.49 (m, 1H), 8.43 (d, J=5.3 Hz, 1H), 8.41-8.33 (m, 1H), 7.97-7.90 (m, 1H), 7.84 (d, J=6.9 Hz, 1H), 7.70-7.62 (m, 1H), 7.30-7.15 (m, 2H), 6.84 (d, J=11.3 Hz, 1H), 6.77 (s, 2H), 6.61-6.53 (m, 1H), 6.56-6.47 (m, 1H), 6.45 (s, 1H), 4.57-4.45 (m, 1H), 4.41-4.15 (m, 3H), 3.71-3.57 (m, 1H), 3.34-3.32 (m, 1H), 2.35 (s, 3H), 1.60-1.53 (m, 3H).

Example 39: 3-(8-chloro-2-(((2R,7aS)-2-fluorotetrahydro-1H-pyrrolizin-7a(5H)-yl)methoxy)-4-methyl-5,6-dihydro-4H-[1, 4]oxazepino[5,6,7-de]quinazolin-9-yl)-5-methyl-4-(trifluoromethyl)aniline

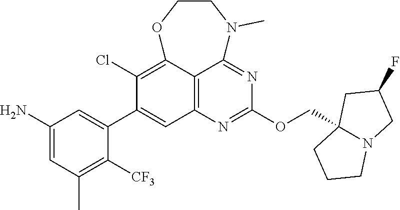 [SMILES: Cc1cc(N)cc(-c2cc3nc(OC[C@@]45CCCN4C[C@H](F)C5)nc4c3c(c2Cl)OCCN4C)c1C(F)(F)F]

### Synthetic Route

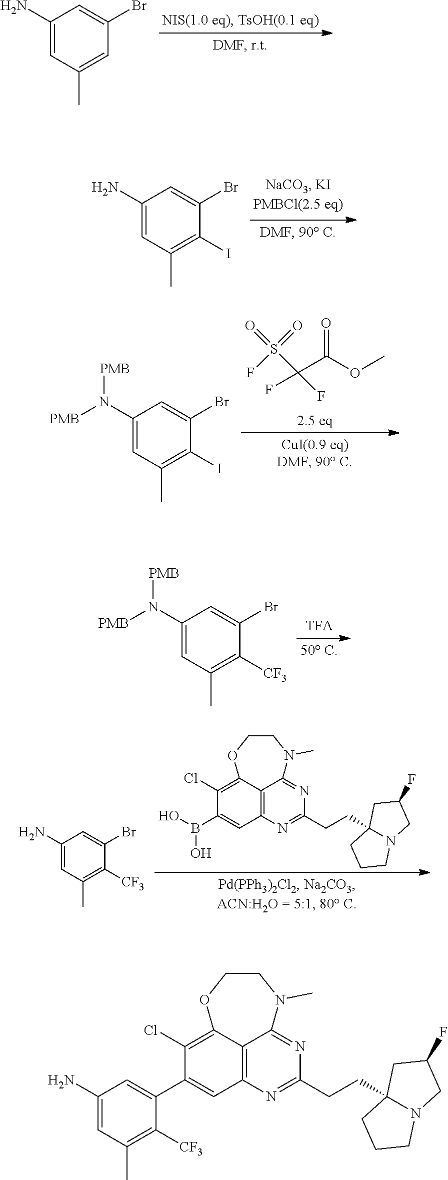

Step 1: 3-bromo-4-iodo-5-methylaniline

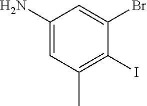

A mixture of 3-bromo-5-methylaniline (10.00 g, 53.74 mmol), N-iodosuccinimide (12.09 g, 53.74 mmol) and p-toluenesulfonic acid (0.93 g, 5.37 mmol) in N,N-dimethylformamide (50 mL) was stirred for 12 h at 25° C. The resulting solution was diluted with water and extracted with ethyl acetate. The organic layers were washed with brine, dried over anhydrous sodium sulfate and concentrated. The resulting residue was purified by reverse phase chromatography (acetonitrile/water) to afford 3-bromo-4-iodo-5-methylaniline (12.00 g, 38.59 mmol, 71.4% yield) as a white solid. LC-MS: (ESI, m/z): 311.0 [M+H]⁺

Step 2: 3-bromo-4-iodo-N,N-bis(4-methoxybenzyl)-5-methylaniline

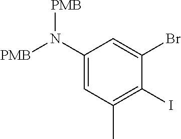

To a mixture of 3-bromo-4-iodo-5-methylaniline (12.00 g, 38.46 mmol) in N,N-dimethylformamide (50 mL) was added sodium carbonate (10.19 g, 96.17 mmol), potassium iodide (3.83 g, 23.08 mmol) and 4-methoxybenzylchloride (13.25 g, 84.62 mmol), the mixture was stirred for 6 h at 90° C. The reaction was then quenched by water. The resulting solution was extracted with ethyl acetate, washed with water, dried over anhydrous sodium sulfate and concentrated under vacuum. The resulting mixture was dissolved in ethanol and stirred for 30 min. The mixture was then filtered to afford of 3-bromo-4-iodo-N,N-bis(4-methoxybenzyl)-5-methylaniline (17.4 g, 31.5 mmol, 81.9% yield) as a white solid. LC-MS: (ESI, m/z): 552.3 [M+H]⁺

Step 3: 3-bromo-N,N-bis(4-methoxybenzyl)-5-methyl-4-(trifluoromethyl)aniline

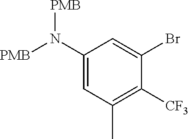

To a mixture of 3-bromo-4-iodo-N,N-bis[(4-methoxyphenyl)methyl]-5-methylaniline (10.00 g, 18.10 mmol) in N,N-dimethylformamide (50 mL) was added cuprous iodide (8.62 g, 45.26 mmol) and methyl 2,2-difluoro-2-sulfoacetate (3.13 g, 16.29 mmol). The mixture was stirred for 6 h at 90° C. The resulting solution was diluted with water and extracted with ethyl acetate. The organic layers were washed with brine, dried over anhydrous sodium sulfate and concentrated. The residue was purified by flash chromatography on silica gel eluting with ethyl acetate/petroleum ether (1/5) to afford 3-bromo-N,N-bis[(4-methoxyphenyl)methyl]-5-methyl-4-(trifluoromethyl)aniline (7.40 g, 14.97 mmol, 82.70% yield) as a light yellow solid. LC-MS: (ESI, m/z): 494.1 [M+H]⁺

Step 4: 3-bromo-5-methyl-4-(trifluoromethyl)aniline

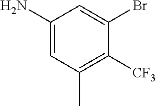

A mixture of 3-bromo-N,N-bis[(4-methoxyphenyl)methyl]-5-methyl-4-(trifluoromethyl)aniline (5.00 g, 10.11 mmol) in trifluoroacetic acid (20 mL) was stirred for 2 h at 50° C. The solvent was removed under vacuum. The resulting solution was diluted with water and adjusted to pH=7 with saturated sodium bicarbonate solution. The resulting solution was extracted with extracted with ethyl acetate, the organic layers were dried over anhydrous sodium sulfate and concentrated. The resulting residue was purified by reverse phase chromatography (acetonitrile/water) to afford 3-bromo-5-methyl-4-(trifluoromethyl)aniline (452.0 mg, 1.78 mmol, 17.6% yield) of as a yellow solid. LC-MS: (ESI, m/z): 254.1 [M+H]⁺

Step 5: 3-(8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-5-methyl-4-(trifluoromethyl)aniline

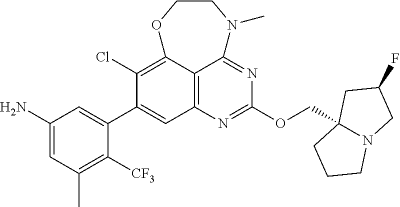 [SMILES: Cc1cc(N)cc(-c2cc3nc(OC[C@@]45CCCN4C[C@H](F)C5)nc4c3c(c2Cl)OCCN4C)c1C(F)(F)F]

Under nitrogen, a solution of (8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)boronic acid (932.3 mg, crude), 3-bromo-5-methyl-4-(trifluoromethyl)aniline (452.0 mg, 1.77 mmol), bis(triphenylphosphine)palladium(II) chloride (249.7 mg, 0.35 mmol) and sodium carbonate (377.1 mg, 3.55 mmol) in acetonitrile (15 mL) and water (3 mL) was added at 80° C. for 1 h. The solvent was removed under vacuum. The residue was purified by flash chromatography on silica gel eluting with dichloromethane/methanol (10/1). The crude product was purified by Prep-HPLC with the following conditions: (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 39% B to 5% B in 10 min, 5% B; Wave Length: 254/220 nm; RT: 8.75 min) to afford 3-(8-chloro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-5-methyl-4-(trifluoromethyl)aniline (32.3 mg, 0.06 mmol, 3.16%). LC-MS: (ESI, m/z): 566.25 [M+H]⁺

Example 39: ¹H NMR (300 MHz, Methanol-d4, ppm) δ 7.00 (s, 1H), 6.63 (s, 1H), 6.23 (s, 1H), 5.37-5.20 (d, J=52.8 Hz, 1H), 4.59-4.55 (m, 2H), 4.29-4.15 (m, 2H), 3.99-3.95 (m, 2H), 3.37 (s, 3H), 3.25-3.17 (m, 3H), 3.05-2.99 (m, 1H), 2.40 (d, J=2.4 Hz, 3H), 2.34-2.10 (m, 3H), 1.99-1.86 (m, 3H).

Example 40a: 6-((R)-8-chloro-10-fluoro-4-methyl-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

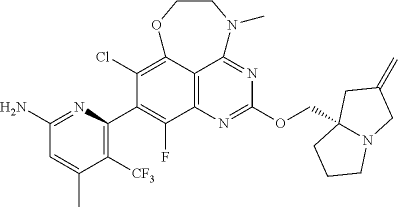

### Synthetic Route

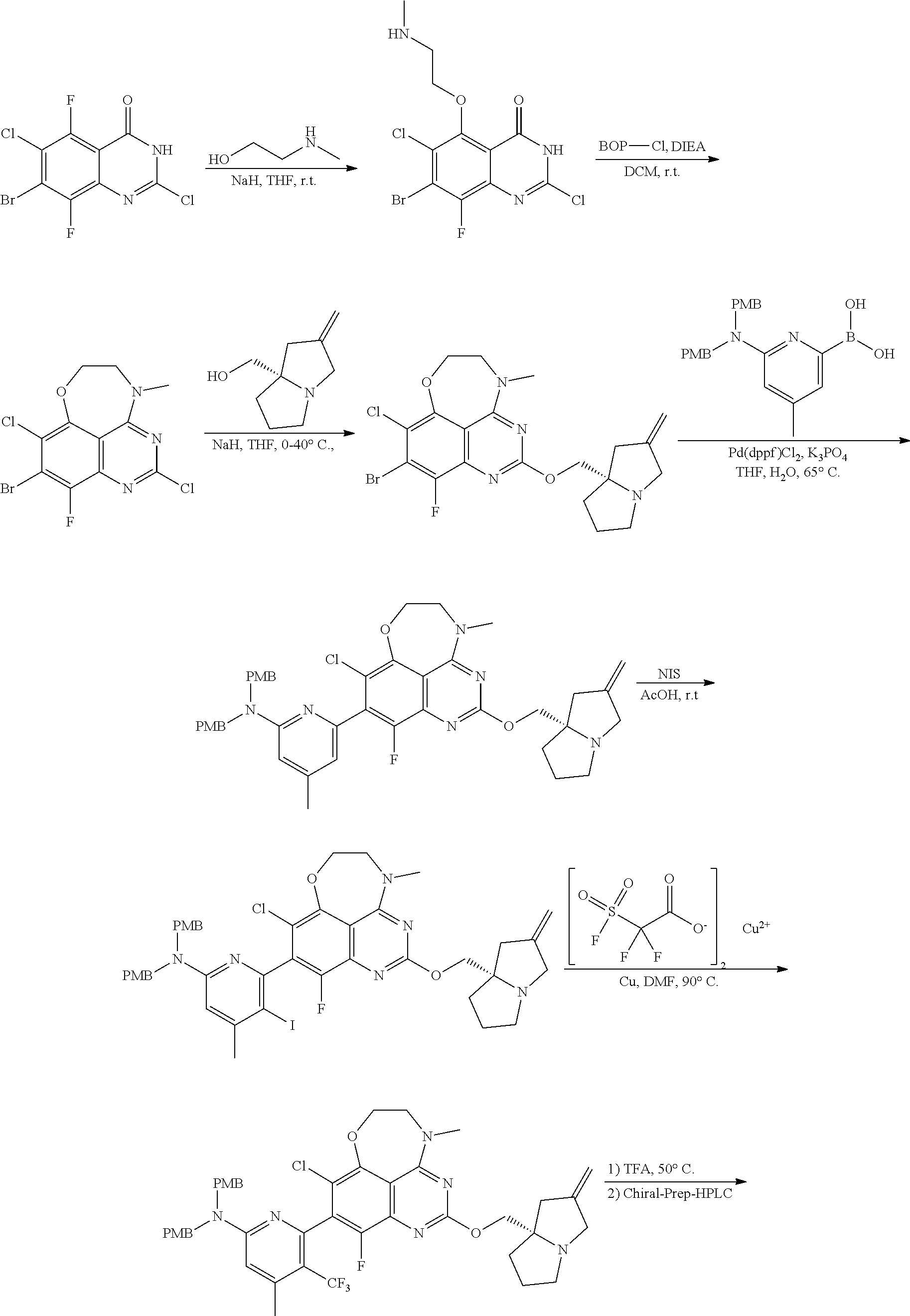

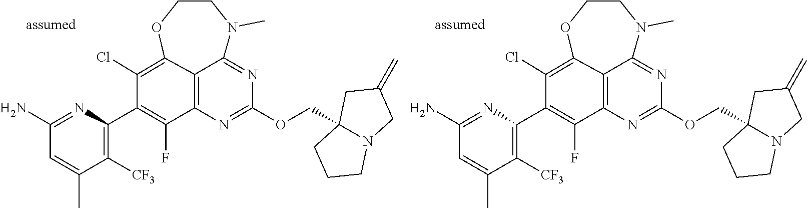

Step 1: 7-bromo-2,6-dichloro-8-fluoro-5-(2-(methylamino)ethoxy)quinazolin-4(3H)-one

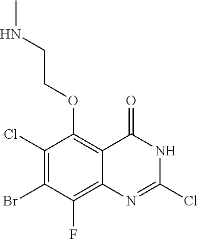 [SMILES: CNCCOc1c(Cl)c(Br)c(F)c2nc(Cl)[nH]c(=O)c12]

A solution of sodium hydride (0.20 g, 5.05 mmol, 60% purity) was added to 2-methylaminoethanol (0.15 g, 2.02 mmol) in tetrahydrofuran (5 mL) cooled to 0° C. Then 7-bromo-2,6-dichloro-5,8-difluoro-3H-quinazolin-4-one (0.74 g, 1.68 mmol) was added and stirred at 0° C. for 5 minutes. Then the mixture warmed to room temperature then stirred for 2 hours. After completion, the reaction was quenched by 1N hydrochloric acid solution. The solvent was concentrated under vacuum. The residue was purified by reverse phase eluting with water/acetonitrile (0-100%) to afford 7-bromo-2,6-dichloro-8-fluoro-5-[2-(methylamino)ethoxy]-3H-quinazolin-4-one (0.41 g, 1.06 mmol, 63.3% yield) as a white solid. LCMS (ESI, m/z): 384.05 [M+H]⁺.

Step 2: 9-bromo-2,8-dichloro-10-fluoro-4-methyl-5,6-dihydro-4H-[1,4]oxazepino[5,6,7-de]quinazoline

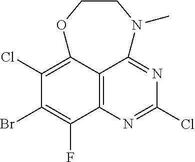 [SMILES: CN1CCOc2c(Cl)c(Br)c(F)c3nc(Cl)nc1c23]

A solution of bis(2-oxo-3-oxazolidinyl)phosphinic chloride (0.81 g, 3.19 mmol) was added to 7-bromo-2,6-dichloro-8-fluoro-5-(2-(methylamino)ethoxy)quinazolin-4(3H)-one (0.41 g, 1.06 mmol) and N,N-diisopropylethylamine (2.06 g, 15.97 mmol) in dichloromethane (3 mL) was stirred at room temperature for 2 hours. After completion, the reaction mixture was diluted with dichloromethane, washed with water, dried over anhydrous sodium sulfate and concentrated under vacuum. The residue was purified by reverse phase eluting with water/acetonitrile (0-100%) to afford 9-bromo-2,8-dichloro-10-fluoro-4-methyl-5,6-dihydro-4H-[1,4]oxazepino[5,6,7-de]quinazoline (0.15 g, 0.41 mmol, 38.4% yield) as a white solid. LCMS (ESI, m/z): 365.85 [M+H]⁺.

Step 3: (S)-9-bromo-8-chloro-10-fluoro-4-methyl-2-((2-methylene-hexahydro-1H-pyrrolizin-7a-yl)methoxy)-5,6-dihydro-4H-[1,4]oxazepino[5,6,7-de]quinazoline

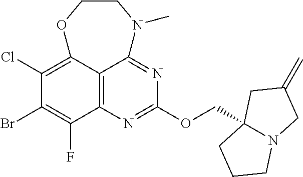 [SMILES: C=C1CN2CCC[C@@]2(COc2nc3c4c(c(Cl)c(Br)c(F)c4n2)OCCN3C)C1]

A solution of sodium hydride (0.65 g, 16.35 mmol, 60% purity) was added to (S)-(2-methylenetetrahydro-1H-pyrrolizin-7a(5H)-yl)methanol (0.42 g, 2.72 mmol) in tetrahydrofuran (5 mL) at 0° C. Then 9-bromo-2,8-dichloro-10-fluoro-4-methyl-5,6-dihydro-4H-[1,4]oxazepino[5,6,7-de]quinazoline (1.0 g, 2.72 mmol) was added and stirred at 0° C. for 5 minutes, warmed to 40° C. Then the mixture was stirred for 1 hour at room temperature. The reaction mixture was quenched with 1N hydrochloric acid solution, diluted with water, extracted with dichloromethane, dried over anhydrous sodium sulfate and concentrated under vacuum. The residue was purified by flash chromatography on silica gel eluting with dichloromethane/methanol (5/1) to afford (S)-9-bromo-8-chloro-10-fluoro-4-methyl-2-((2-methylene-hexahydro-1H-pyrrolizin-7a-yl)methoxy)-5,6-dihydro-4H-[1,4]oxazepino[5,6,7-de]quinazoline (430.0 mg, 0.89 mmol, 32.6% yield) as a white solid. LCMS (ESI, m/z): 483.0 [M+H]⁺.

Step 4: (S)-6-(8-chloro-10-fluoro-4-methyl-2-((2-methylene-hexahydro-1H-pyrrolizin-7a-yl)methoxy)-5,6-dihydro-4H-[1,4]oxazepino[5,6,7-de]quinazolin-9-yl)-N,N-bis(4-methoxybenzyl)-4-methylpyridin-2-amine

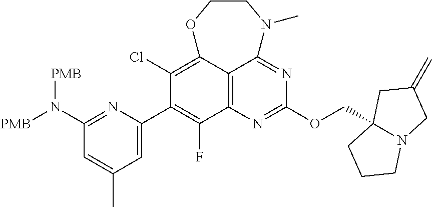

Under nitrogen, a solution of (S)-9-bromo-8-chloro-10-fluoro-4-methyl-2-((2-methylene-hexahydro-1H-pyrrolizin-7a-yl)methoxy)-5,6-dihydro-4H-[1,4]oxazepino[5,6,7-de]quinazoline (0.20 g, 0.41 mmol), [6-[bis[(4-methoxyphenyl)methyl]amino]-4-methyl-2-pyridyl]boronic acid (0.54 g, 0.83 mmol), 1,1′-bis(diphenylphosphino)ferrocene-palladium(II)dichloride dichloromethane complex (0.03 g, 0.04 mmol) and potassium phosphate (0.26 g, 1.24 mmol) in tetrahydrofuran (3 mL) and water (0.60 mL) was stirred for 60 minutes at 65° C. The reaction mixture was diluted with dichloromethane, washed with water and dried over anhydrous sodium sulfate and concentrated under vacuum. The residue was purified by flash chromatography on silica gel eluting with petroleum ether/ethyl acetate (1/1) to afford (S)-6-(8-chloro-10-fluoro-4-methyl-2-((2-methylene-hexahydro-1H-pyrrolizin-7a-yl)methoxy)-5,6-dihydro-4H-[1,4]oxazepino[5,6,7-de]quinazolin-9-yl)-N,N-bis(4-methoxybenzyl)-4-methylpyridin-2-amine (0.17 g, 0.23 mmol, 54.7% yield) as a yellow solid. LCMS (ESI, m/z): 751.25 [M+H]⁺.

Step 5: 6-(8-chloro-10-fluoro-4-methyl-2-(((S)-2-methylene-hexahydro-1H-pyrrolizin-7a-yl)methoxy)-5,6-dihydro-4H-[1,4]oxazepino[5,6,7-de]quinazolin-9-yl)-5-iodo-N,N-bis(4-methoxybenzyl)-4-methylpyridin-2-amine

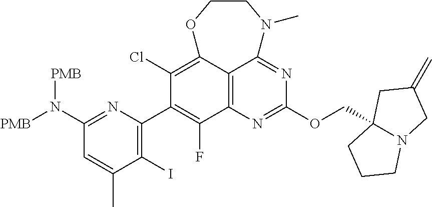

A solution of (S)-6-(8-chloro-10-fluoro-4-methyl-2-((2-methylene-hexahydro-1H-pyrrolizin-7a-yl)methoxy)-5,6-dihydro-4H-[1,4]oxazepino[5,6,7-de]quinazolin-9-yl)-N,N-bis(4-methoxybenzyl)-4-methylpyridin-2-amine (0.35 g, 0.47 mmol) and N-iodosuccinimide (0.10 g, 0.47 mmol) in acetic acid (5 mL) was stirred at room temperature for 0.5 hours. After completion, the reaction was quenched with saturated sodium thiosulfate solution. The reaction mixture was diluted with ethyl acetate, washed with water and dried over anhydrous sodium sulfate and concentrated under vacuum. The residue was purified by reverse phase eluting with water/acetonitrile to afford 6-(8-chloro-10-fluoro-4-methyl-2-(((S)-2-methylene-hexahydro-1H-pyrrolizin-7a-yl)methoxy)-5,6-dihydro-4H-[1,4]oxazepino[5,6,7-de]quinazolin-9-yl)-5-iodo-N,N-bis(4-methoxybenzyl)-4-methylpyridin-2-amine (0.26 g, 0.30 mmol, 63.6% yield) as a yellow solid. LCMS (ESI, m/z): 877.2 [M+H]⁺.

Step 5: 6-(8-chloro-10-fluoro-4-methyl-2-(((S)-2-methylene-hexahydro-1H-pyrrolizin-7a-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

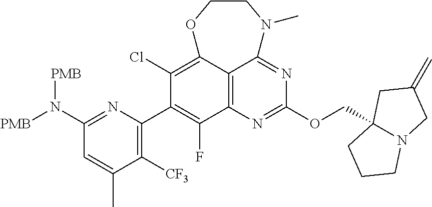

Under nitrogen, a solution of 6-(8-chloro-10-fluoro-4-methyl-2-(((S)-2-methylene-hexahydro-1H-pyrrolizin-7a-yl)methoxy)-5,6-dihydro-4H-[1,4]oxazepino[5,6,7-de]quinazolin-9-yl)-5-iodo-N,N-bis(4-methoxybenzyl)-4-methylpyridin-2-amine (250 mg, 0.29 mmol) and copper (54 mg, 0.86 mmol) in N,N-dimethylformamide (5 mL) was stirred 5 minutes at 0° C. Then bis[(2,2-difluoro-2-fluorosulfonyl-acetyl)oxy]copper (357 mg, 0.86 mmol) was added and stirred at 0° C. for 10 minutes. Then the solution was warmed to room temperature and then under 90° C. and stirred for 1 hour. After completion, the resulting reaction mixture was filtered, the filtrate was diluted with ethyl acetate, washed with water, dried over anhydrous sodium sulfate and concentrated under vacuum. The residue was purified by reverse phase with water (0.1% TFA)/acetonitrile to afford 6-(8-chloro-10-fluoro-4-methyl-2-(((S)-2-methylene-hexahydro-1H-pyrrolizin-7a-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (70.0 mg, 0.09 mmol, 30% yield) as a yellow solid. LCMS (ESI, m/z): 819.6[M+H]⁺.

Step 6: 6-((R)-8-chloro-10-fluoro-4-methyl-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

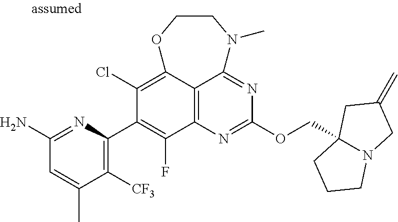

A solution of 6-(8-chloro-10-fluoro-4-methyl-2-(((S)-2-methylene-hexahydro-1H-pyrrolizin-7a-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (65 mg, 0.08 mmol) in trifluoroacetic acid (3 mL) was stirred at 50° C. for 12 hours. The solvent was concentrated under vacuum. The residue was purified by Prep-HPLC (Column: XBridge Prep OBD C18 Column, 30*150 mm, 5 μm; Mobile Phase A: Water (10 MMOL/L NH4HCO3), Mobile Phase B: ACN; Flow rate: 60 mL/min: Gradient: 44% B to 74% B in 7 min; Wave Length: 254 nm; RT1(min): 6.5; Number Of Runs: 0) and Chiral-Prep-HPLC (Column: CHIRAL ART Cellulose-SC, 2*25 cm, 5 μm; Mobile Phase A: Hex (0.5% 2M NH3-MeOH)-HPLC, Mobile Phase B: EtOH-HPLC; Flow rate: 20 mL/min; Gradient: 50% B to 50% B in 9 min; Wave Length: 220/254 nm; RT1(min): 5.666; RT2(min): 8.112; Sample Solvent: EtOH-HPLC; Injection Volume: 2 mL; Number Of Runs: 3) to afford 6-((R)-8-chloro-10-fluoro-4-methyl-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (6.5 mg, 0.01 mmol, 14.2% yield). LCMS: (ESI, m/z): 579.15 [M+H]⁺.

Example 40a: ¹H NMR (300 MHz, Methanol-d4, ppm) δ 6.59 (s, 1H), 5.00 (s, 2H), 4.75-4.60 (m, 2H), 4.36-4.21 (m, 2H), 4.04-3.92 (m, 2H), 3.82-3.70 (m, 1H), 3.41 (s, 3H), 3.38 (s, 1H), 3.22-3.15 (m, 1H), 2.88-2.70 (m, 2H), 2.55-2.39 (m, 4H), 2.22-2.10 (m, 1H), 2.05-1.78 (m, 3H)

Example 41a & 41b: 6-((R)-8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine & 6-((S)-8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine

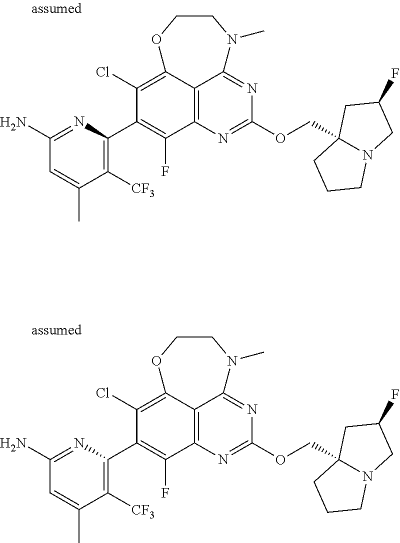

### Synthetic Route

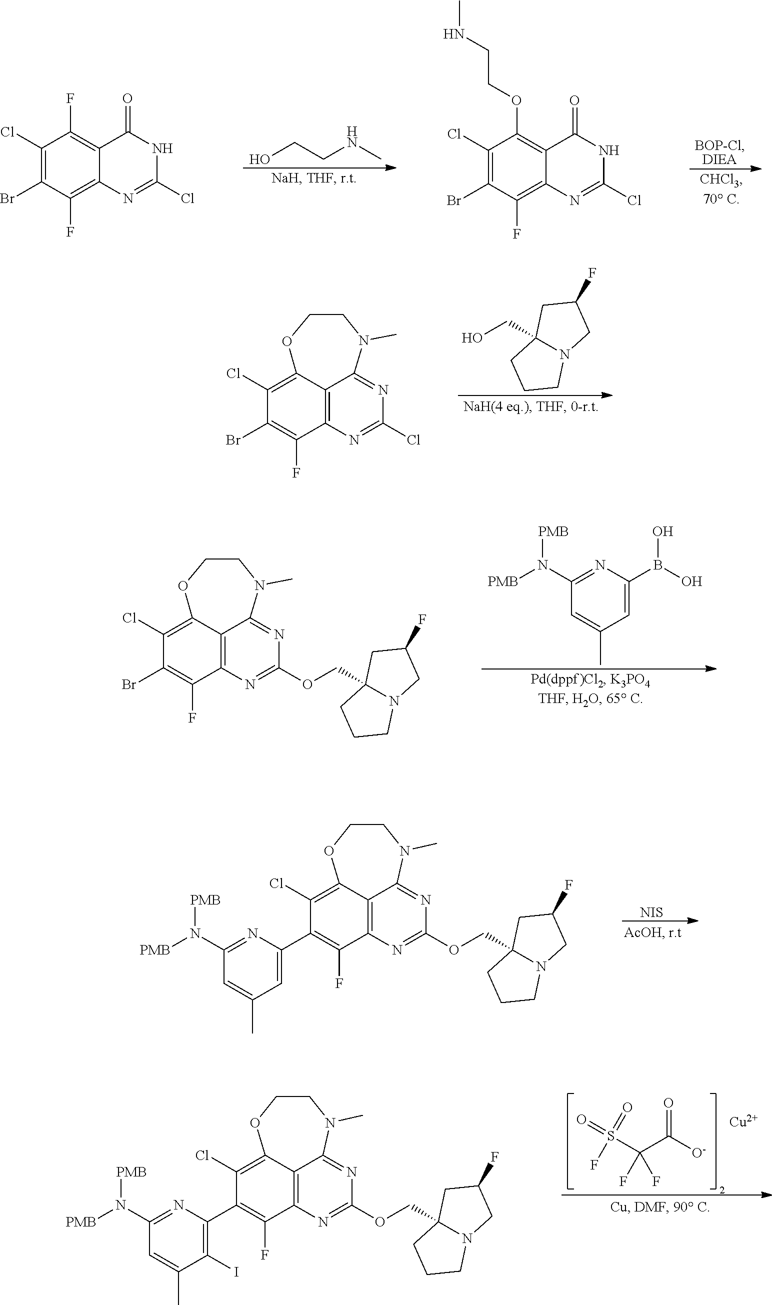

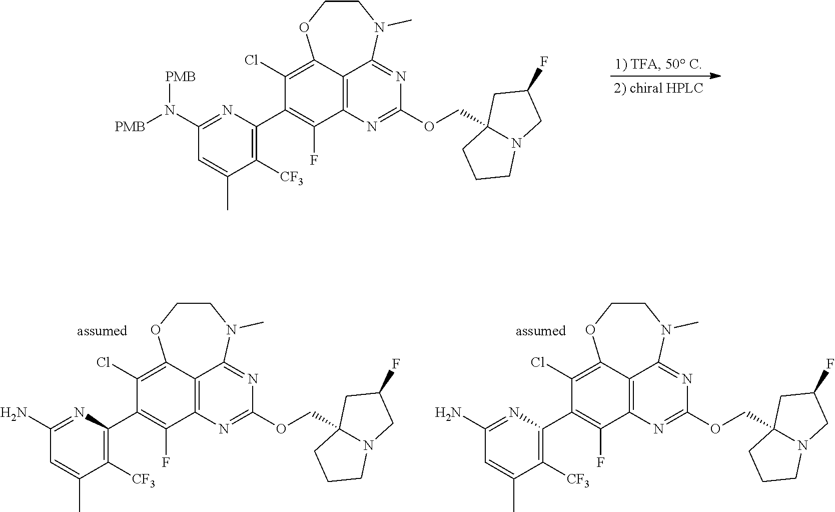

Step 1: 7-bromo-2,6-dichloro-8-fluoro-5-(2-(methylamino)ethoxy)quinazolin-4(3H)-one

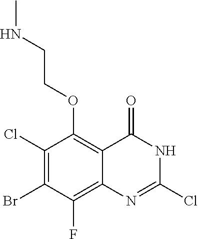 [SMILES: CNCCOc1c(Cl)c(Br)c(F)c2nc(Cl)[nH]c(=O)c12]

A solution of sodium hydride (0.20 g, 5.05 mmol, 60% purity) was added to 2-methylaminoethanol (0.15 g, 2.02 mmol) in tetrahydrofuran (5 mL) at 0° C. Then 7-bromo-2,6-dichloro-5,8-difluoro-3H-quinazolin-4-one (0.74 g, 1.68 mmol) was added and stirred at 0° C. for 5 minutes, allowed to warm to room temperature then stirred for 2 hours. The reaction was quenched by 1N hydrochloric acid solution. The solvent was concentrated under vacuum. The residue was purified by reverse phase eluting with water/acetonitrile (0-100%) to afford 7-bromo-2,6-dichloro-8-fluoro-5-(2-(methylamino)ethoxy)quinazolin-4(3H)-one (0.41 g, 1.06 mmol, 63.3% yield) as a white solid. LCMS (ESI, m/z): 384.05 [M+H]⁺.

Step 9-bromo-2,8-dichloro-10-fluoro-4-methyl-5,6-dihydro-4H-[1,4]oxazepino[5,6,7-de]quinazoline

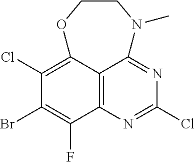 [SMILES: CN1CCOc2c(Cl)c(Br)c(F)c3nc(Cl)nc1c23]

A solution of bis(2-oxo-3-oxazolidinyl)phosphinic chloride (0.81 g, 3.19 mmol) was added to 7-bromo-2,6-dichloro-8-fluoro-5-(2-(methylamino)ethoxy)quinazolin-4(3H)-one (0.41 g, 1.06 mmol) and N,N-diisopropylethylamine (2.06 g, 15.97 mmol) in chloroform (3 mL) at 25° C. and stirred at 70° C. for 2 hours. After completion, the reaction mixture was diluted with dichloromethane, washed with water, dried over anhydrous sodium sulfate and concentrated under vacuum. The residue was purified by reverse phase eluting with water/acetonitrile (0-100%) to afford 9-bromo-2,8-dichloro-10-fluoro-4-methyl-5,6-dihydro-4H-[1,4]oxazepino[5,6,7-de]quinazoline (0.15 g, 0.41 mmol, 38.4% yield) as a white solid. LCMS (ESI, m/z): 365.85 [M+H]⁺.

Step 3: 9-bromo-8-chloro-10-fluoro-2-(((2R,7aS)-2-fluoro-hexahydro-1H-pyrrolizin-7a-yl)methoxy)-4-methyl-5,6-dihydro-4H-[1,4]oxazepino[5,6,7-de]quinazoline

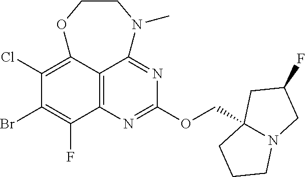 [SMILES: CN1CCOc2c(Cl)c(Br)c(F)c3nc(OC[C@@]45CCCN4C[C@H](F)C5)nc1c23]

A solution of sodium hydride (0.26 g, 6.54 mmol, 60% purity) was added to ((2R,7aS)-2-fluorotetrahydro-1H-pyrrolizin-7a(5H)-yl)methanol (0.19 g, 1.2 mmol) in tetrahydrofuran (10 mL) at 0° C. Then 9-bromo-2,8-dichloro-10-fluoro-4-methyl-5,6-dihydro-4H-[1,4]oxazepino[5,6,7-de]quinazoline (0.40 g, 1.09 mmol) was added and stirred at 0° C. for 5 minutes. Then the solution was allowed to warm to room temperature and stirred for 12 hours. The reaction mixture was diluted with 1N hydrochloric acid solution, extracted with dichloromethane, dried over anhydrous sodium sulfate and concentrated under vacuum. The residue was purified by flash chromatography on silica gel eluting with dichloromethane/methanol (10/1) to afford 9-bromo-8-chloro-10-fluoro-2-(((2R,7aS)-2-fluoro-hexahydro-1H-pyrrolizin-7a-yl)methoxy)-4-methyl-5,6-dihydro-4H-[1,4]oxazepino[5,6,7-de]quinazoline (250.0 mg, 0.51 mmol, 46.8% yield) as a white solid. LCMS (ESI, m/z): 491.05 [M+H]⁺.

Step 4: 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methylpyridin-2-amine

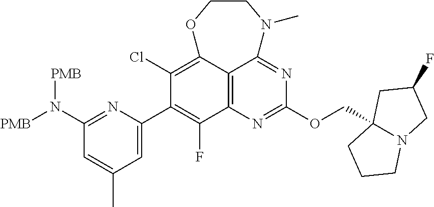

Under nitrogen, a solution of 9-bromo-8-chloro-10-fluoro-2-(((2R,7aS)-2-fluoro-hexahydro-1H-pyrrolizin-7a-yl)methoxy)-4-methyl-5,6-dihydro-4H-[1,4]oxazepino[5,6,7-de]quinazoline (0.30 g, 0.61 mmol), [6-[bis[(4-methoxyphenyl)methyl]amino]-4-methyl-2-pyridyl]boronic acid (0.80 g, 1.23 mmol), 1,1′-bis(diphenylphosphino)ferrocene-palladium(II)dichloride dichloromethane complex (0.05 g, 0.06 mmol) and potassium phosphate (0.39 g, 1.84 mmol) in tetrahydrofuran (3 mL) and water (0.6 mL) was stirred for 60 minutes at 65° C. After completion, the reaction mixture was diluted with dichloromethane, washed with water, dried over anhydrous sodium sulfate and concentrated under vacuum. The residue was purified by flash chromatography on silica gel eluting with dichloromethane/methanol (10/1) to afford 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methylpyridin-2-amine (0.30 g, 0.40 mmol, 64.7% yield) as a yellow solid. LCMS (ESI, m/z): 757.3 [M+H]⁺.

Step 5: 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-5-iodo-N,N-bis(4-methoxybenzyl)-4-methylpyridin-2-amine

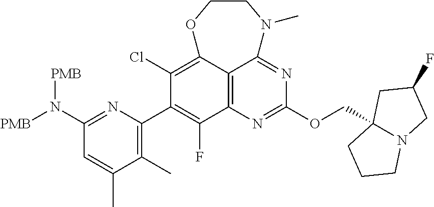

A solution of 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methylpyridin-2-amine (0.29 g, 0.38 mmol) and N-iodosuccinimide (0.09 g, 0.42 mmol) in acetic acid (5.0 mL) was stirred at 25° C. for 0.5 hours. After completion, the reaction was quenched with saturated sodium thiosulfate solution. The reaction mixture was diluted with ethyl acetate. The organic layer was washed with water. The organic layer was dried over anhydrous sodium sulfate and concentrated under vacuum. The residue was purified by flash chromatography on silica gel eluting with dichloromethane/methanol (20/1) to afford 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-5-iodo-N,N-bis(4-methoxybenzyl)-4-methylpyridin-2-amine (0.38 g, 0.34 mmol, 89.9% yield) as a yellow solid. LCMS (ESI, m/z): 883.35 [M+H]⁺.

Step 6: 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

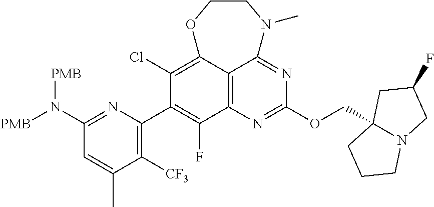

Under nitrogen, a solution of 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-5-iodo-N,N-bis(4-methoxybenzyl)-4-methylpyridin-2-amine (370 mg, 0.34 mmol) and copper (639 mg, 10.05 mmol) in N,N-dimethylformamide (10 mL) was stirred 5 minutes at 0° C. Then bis[(2,2-difluoro-2-fluorosulfonyl-acetyl)oxy]copper (4.20 g, 10.05 mmol) was added and stirred at 0° C. for 10 minutes. Then the solution was warmed to room temperature and then under 90° C. and stirred for 10 minutes. After completion, the resulting reaction mixture was filtered, the filtrate was diluted with ethyl acetate, washed with water, dried over anhydrous sodium sulfate and concentrated under vacuum. The residue was purified by flash chromatography on silica gel eluting with dichloromethane/methanol (20/1) to afford 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (280 mg, 0.24 mmol, 70.9% yield) as a yellow solid. LCMS (ESI, m/z): 825.4 [M+H]⁺.

Step 7: 6-((R)-8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine & 6-((S)-8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine

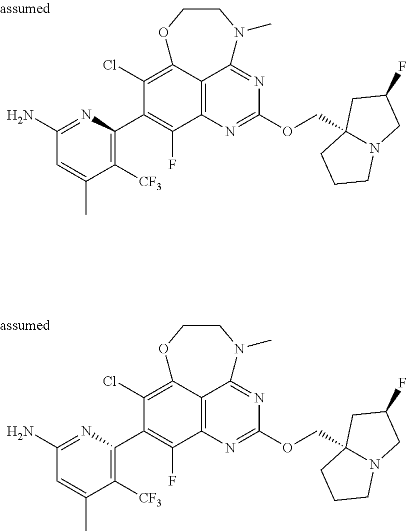

A solution of 6-(8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (270.0 mg, 0.23 mmol) in trifluoroacetic acid (5 mL) was stirred at 50° C. for 10 hours. After completion, the solvent was concentrated under vacuum. The residue was purified by reverse phase eluting with water/acetonitrile and Pre-HPLC (Column: XBridge Prep OBD C18 Column, 30*150 mm, 5 μm; Mobile Phase A: Water (10 MMOL/L NH4HCO3), Mobile Phase B: ACETONITRILE: Flow rate: 60 mL/min; Gradient: 36% B to 58% B in 9 min, 58% B: Wave Length: 254/220 nm; RT1(min): 8.8; Number Of Runs: 0) and CHIRAL_HPLC (Column: CHIRALPAK IE, 2*25 cm, 5 μm; Mobile Phase A: Hex (0.5% 2M NH3-MeOH)-HPLC, Mobile Phase B: EtOH-HPLC; Flow rate: 18 mL/min; Gradient: 50% B to 50% B in 23 min; Wave Length: 220/254 nm; RT1(min): 12.096; RT2(min): 17.544; Sample Solvent: EtOH-HPLC; Injection Volume: 1.5 mL; Number Of Runs: 5) to afford 6-((R)-8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine (17.8 mg, 0.03 mmol, 13.3% yield) and 6-((S)-8-chloro-10-fluoro-2-(((2R,7aS)-2-fluorotetrahydro-1H-pyrrolizin-7a(5H)-yl)methoxy)-4-methyl-5,6-dihydro-4H-[1,4]oxazepino[5,6,7-de]quinazolin-9-yl)-4-methyl-5-(trifluoromethyl)pyridin-2-amine (21.3 mg, 0.04 mmol, 15.9% yield). The stereo chemistry of title compounds was arbitrarily assigned

Example 41a: ¹H NMR (300 MHz, DMSO-d6, ppm) δ 6.81 (s, 2H), 6.47 (s, 1H), 5.29 (d, J=53.6 Hz, 1H), 4.70-4.45 (m, 2H), 4.11 (d, J=10.2 Hz, 1H), 4.01-3.83 (m, 3H), 3.29 (s, 3H) 3.16-3.02 (m, 2H), 3.00-2.97 (m, 1H), 2.91-2.73 (m, 1H), 2.36 (d, J=1.5 Hz, 3H), 2.23-2.11 (m, 1H), 2.11-1.93 (m, 2H), 1.93-1.67 (m, 3H). LCMS (ESI, m/z): 585.30 [M+H]⁺. Chiral HPLC: Column: CHIRALPAK IE-3, 4.6*50 mm 3 um; Mobile Phase A: Hex (0.1% DEA):EtOH=50:50; Flow rate: 1 mL/min; Injection Volume: 5 ul ml; Retention time: 2.930 min (First peak)

Example 41b: ¹H NMR (300 MHz, DMSO-d6, ppm) δ 6.81 (s, 2H), 6.47 (s, 1H), 5.28 (d, J=53.6 Hz, 1H), 4.67-4.41 (m, 2H), 4.21-3.99 (m, 2H), 3.99-3.87 (m, 2H), 3.35 (s, 3H), 3.15-3.03 (m, 2H), 3.06-2.97 (m, 1H), 2.94-2.76 (m, 1H), 2.35 (d, J=1.5 Hz, 3H), 2.20-2.12 (m, 1H), 2.12-1.92 (m, 2H), 1.92-1.70 (m, 3H). LCMS (ESI, m/z): 585.30 [M+H]⁺. Chiral HPLC: Column: CHIRALPAK IE-3, 4.6*50 mm 3 um; Mobile Phase A: Hex (0.1% DEA):EtOH=50:50; Flow rate: 1 mL/min; Injection Volume: 5 ul mL; Retention time: 5.530 min (Second peak)

Example 42a&42b: (R)-6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & (S)-6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

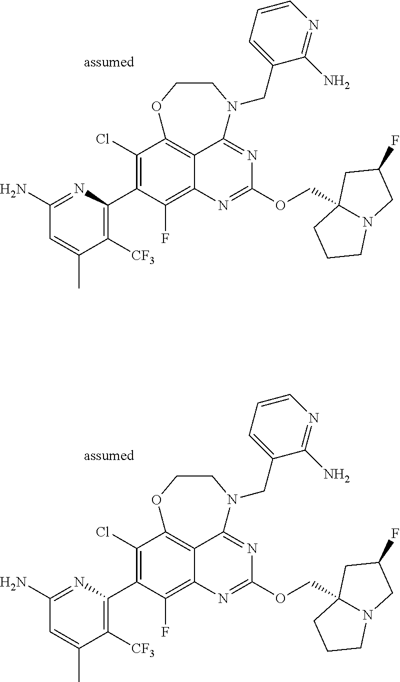

### Synthetic Route

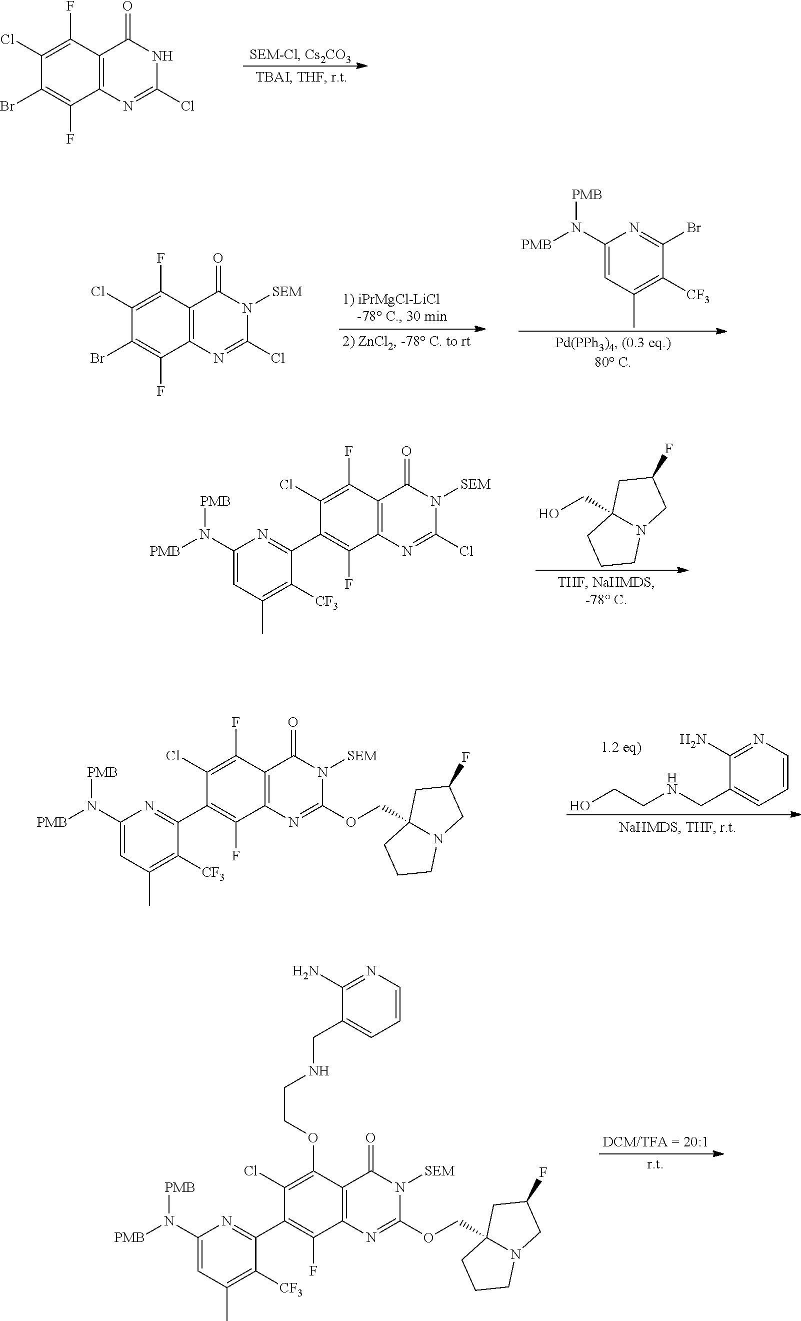

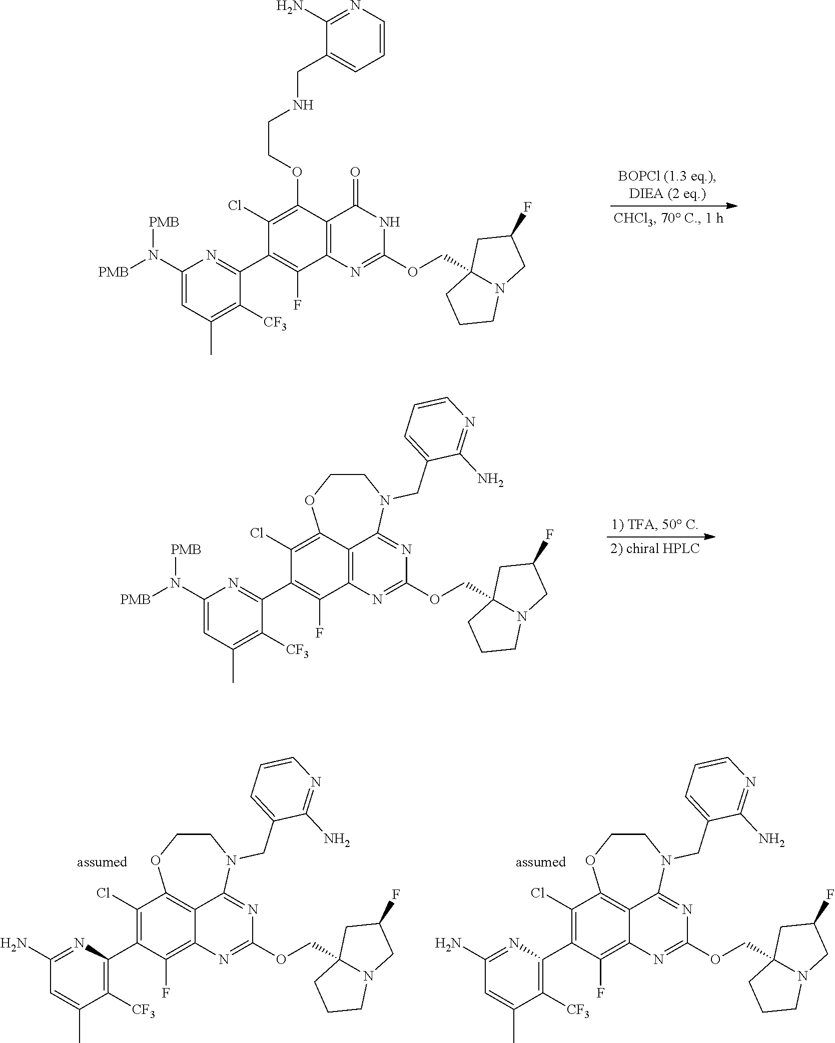

Step 1: 7-bromo-2,6-dichloro-5,8-difluoro-3-((2-(trimethylsilyl)ethoxy)methyl)quinazolin-4(3H)-one

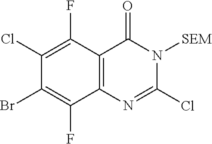 [SMILES: C[Si](C)(C)CCOCn1c(Cl)nc2c(F)c(Br)c(Cl)c(F)c2c1=O]

The solution of 7-bromo-2,6-dichloro-5,8-difluoro-3H-quinazolin-4-one (10.00 g, 30.30 mmol), cesium carbonate (19.90 g, 60.60 mmol) and tetrabutylammonium iodide (1.10 g, 3.00 mmol) in tetrahydrofuran (200 mL) was added 2-(trimethylsilyl)ethoxymethyl chloride (8.10 g, 48.50 mmol) at 0° C. for 5 minutes and stirred at room temperature for 3 hours. After completion, after filtration, the filtrate was concentrated under reduced pressure. The residue was purified by flash chromatography on silica gel eluting with petrol ether/ethyl acetate (10/1) to afford 7-bromo-2,6-dichloro-5,8-difluoro-3-((2-(trimethylsilyl)ethoxy)methyl)quinazolin-4(3H)-one (10.00 g, 21.73 mmol, 71.7% yield) as a white solid. LCMS (ESI, m/z): 401.2 [M+H−58]⁺.

Step 2: 7-(6-(bis(4-methoxybenzyl)amino)-4-methyl-3-(trifluoromethyl)pyridin-2-yl)-2,6-dichloro-5,8-difluoro-3-((2-(trimethylsilyl)ethoxy)methyl)quinazolin-4(3H)-one

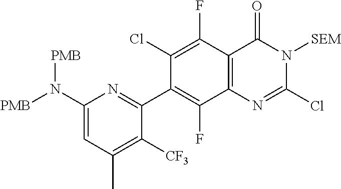

Under nitrogen, 7-bromo-2,6-dichloro-5,8-difluoro-3-(2-trimethylsilylethoxymethyl)quinazolin-4-one (1.00 g, 2.20 mmol) in tetrahydrofuran (10 mL) was added isopropylmagnesium chloride-lithium chloride complex (1.60 mL, 2.20 mmol, 1.3 M in THF) at −78° C. The reaction was stirred at −78° C. for 20 minutes. Then zinc chloride (3.20 mL, 6.50 mmol, 2 M in MeTHF) was added and stirred at −78° C. for 5 minutes and stirred at room temperature for 20 min. The mixture was transferred into a mixture of tetrakis(triphenylphosphine)palladium (0.75 g, 0.65 mmol) and 6-bromo-N,N-bis[(4-methoxyphenyl)methyl]-4-methyl-5-(trifluoromethyl)pyridin-2-amine (1.10 g, 2.20 mmol) in tetrahydrofuran (10 mL) and stirred at 80° C. for 1 h. After completion, the reaction was quenched by saturated ammonium chloride solution and extracted with dichloromethane. The combined organic layers were washed with saturated sodium chloride solution and dried over anhydrous sodium sulfate and concentrated under vacuum. The residue was purified by flash chromatography on silica gel eluting with petrol ether/ethyl acetate (5/1) to afford 7-(6-(bis(4-methoxybenzyl)amino)-4-methyl-3-(trifluoromethyl)pyridin-2-yl)-2,6-dichloro-5,8-difluoro-3-((2-(trimethylsilyl)ethoxy)methyl)quinazolin-4(3H)-one (0.32 g, 0.40 mmol, 18.5% yield) as a white solid. LCMS (ESI, m/z): 795.2 [M+H]⁺.

Step 3: 7-(6-(bis(4-methoxybenzyl)amino)-4-methyl-3-(trifluoromethyl)pyridin-2-yl)-6-chloro-5,8-difluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one

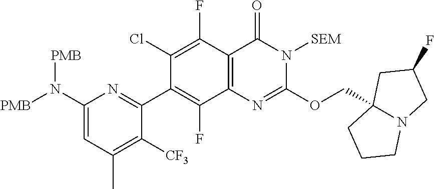

Under nitrogen, to a solution of ((2R,7aS)-2-fluorotetrahydro-1H-pyrrolizin-7a(5H)-yl)methanol (2.40 g, 15.08 mmol) in tetrahydrofuran (40 mL) was added sodium bis(trimethylsilyl)amide (15.08 mL, 15.08 mmol, 1 M in THF) at 0° C. The reaction was stirred at 0° C. for 10 minutes. The mixture was transferred into a mixture of 7-(6-(bis(4-methoxybenzyl)amino)-4-methyl-3-(trifluoromethyl)pyridin-2-yl)-2,6-dichloro-5,8-difluoro-3-((2-(trimethylsilyl)ethoxy)methyl)quinazolin-4(3H)-one (4.00 g, 5.03 mmol) in tetrahydrofuran (40 mL) and stirred at −78° C. for 0.5 h. The reaction was quenched by saturated ammonium chloride solution and extracted with dichloromethane. The combined organic layers were washed with saturated sodium chloride and dried over anhydrous sodium sulfate and concentrated under vacuum. The residue was purified by flash chromatography on silica gel eluting with dichloromethane/methanol (20/1) to afford 7-(6-(bis(4-methoxybenzyl)amino)-4-methyl-3-(trifluoromethyl)pyridin-2-yl)-6-chloro-5,8-difluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (2.40 g, 2.61 mmol, 52% yield) as a colorless solid. LCMS: (ESI, m/z): 918.45 [M+H]⁺.

Step 4: 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one

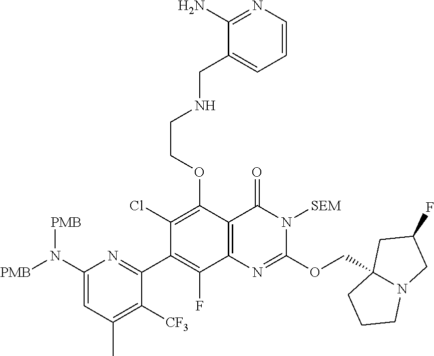

Under nitrogen, 2-(((2-aminopyridin-3-yl)methyl)amino)ethan-1-ol (0.54 g, 3.26 mmol)) in tetrahydrofuran (5 mL) was added sodium bis(trimethylsilyl)amide (3.26 mL, 3.26 mmol, 1 M in the THF) and stirred at 0° C. for 5 minutes. The mixture was transferred into a mixture of 7-(6-(bis(4-methoxybenzyl)amino)-4-methyl-3-(trifluoromethyl)pyridin-2-yl)-6-chloro-5,8-difluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (2.30 g, 2.50 mmol) in tetrahydrofuran (20 mL) and stirred at room temperature for 5 h. The reaction was quenched by saturated ammonium chloride solution and extracted with dichloromethane. The combined organic layers were washed with saturated sodium chloride and dried over anhydrous sodium sulfate and concentrated under vacuum. The residue was purified by flash chromatography on silica gel eluting with dichloromethane/methanol (20/1) to afford 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3h)-one (1.20 g, 1.13 mmol, 45% yield) as a white solid. LCMS: (ESI, m/z): 1065.3 [M+H]⁺.

Step 5: 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4(3H)-one

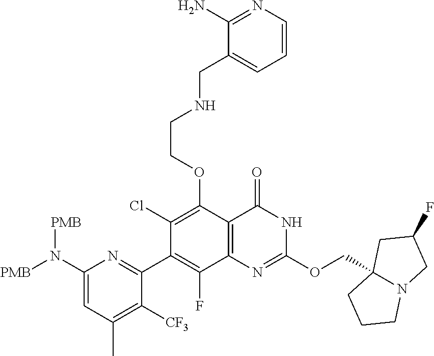

A solution of 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (1.00 g, 0.94 mmol) in dichloromethane (20 mL) and trifluoroacetic acid (1 mL) was stirred at room temperature for 0.5 hours. After completion, the solvent was concentrated under vacuum. The residue was purified by flash chromatography on silica gel eluting with dichloromethane/methanol (10/1) to afford 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4(3H)-one (700.0 mg, 0.75 mmol, 79.7% yield) as a yellow solid. LCMS: (ESI, m/z): 935.2 [M+H]⁺.

Step 6: 6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

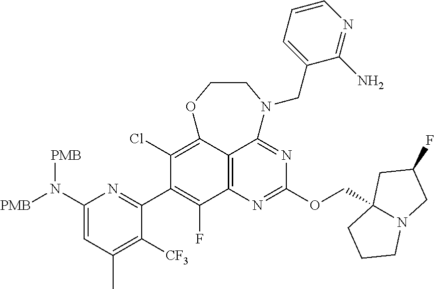

A solution of 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)quinazolin-4(3H)-one (0.65 g, 0.69 mmol), bis(2-oxo-3-oxazolidinyl)phosphinic chloride (0.23 g, 0.90 mmol) and N,N-diisopropylethylamine (0.18 g, 1.40 mmol) in chloroform (5 mL) was stirred at 70° C. for 5 hours. The solvent was concentrated under vacuum. The residue was purified by flash chromatography on silica gel eluting with dichloromethane/methanol (20/1) to afford 6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (280.0 mg, 0.31 mmol, 43.9% yield) as a yellow solid. LCMS: (ESI, m/z): 917.25 [M+H]⁺.

Step 7: (R)-6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & (S)-6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

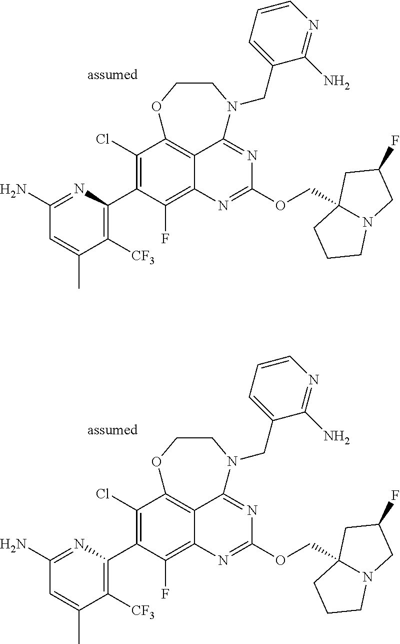

A solution of 6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (280.0 mg, 0.31 mmol) in trifluoroacetic acid (10 mL) was stirred at 50° C. for 5 hours. After completion, the solvent was concentrated under vacuum and adjusted to pH >7 with N,N-diisopropylethylamine. The residue was purified by reverse phase eluting with water/acetonitrile and CHIRAL_HPLC (Column: CHIRAL ART Cellulose-SC, 2*25 cm, 5 μm; Mobile Phase A: Hex (0.5% 2M NH3-MeOH)-HPLC, Mobile Phase B: EtOH-HPLC; Flow rate: 20 mL/min; Gradient: 50% B to 50% B in 8 min; Wave Length: 220/254 nm; RT1(min): 4.94: RT2(min): 6.83; Sample Solvent: EtOH-HPLC; Injection Volume: 1 mL; Number Of Runs: 3) to afford (R)-6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (46.7 mg, 0.069 mmol, 22.6% yield) and (S)-6-(4-((2-aminopyridin-3-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (43.5 mg, 0.064 mmol, 21% yield). The stereo chemistry of title compounds was arbitrarily assigned

Example 42a: ¹H NMR (300 MHz, Methanol-d4, ppm) δ 7.98-7.91 (m, 1H), 7.38-7.29 (m, 1H), 6.78-6.69 (m, 1H), 6.67 (s, 1H), 5.25 (d, J=53.6 Hz, 1H), 5.00 (s, 2H), 4.72-4.55 (m, 2H), 4.21-4.09 (m, 2H), 4.04-3.92 (m, 2H), 3.30-3.10 (m, 3H), 3.09-2.90 (m, 1H), 4.46 (d, J=1.5 Hz, 3H), 2.31-2.11 (m, 2H), 2.10-1.81 (m, 4H); LCMS: (ESI, m/z): 677.1 [M+H]⁺.

Chiral HPLC: Column: CHIRALPAK IC-3, 4.6*50 mm 3 um; Mobile Phase A: Hex (0.1% DEA):EtOH=50:50; Flow rate: 1 mL/min; Injection Volume: 5 ul mL; Retention time: 1.070 min (First peak).

Example 42b: ¹H NMR (300 MHz, Methanol-d4, ppm) δ 7.90 (dd, J=5.2, 1.8 Hz, 1H), 7.44 (dd, J=7.4, 1.8 Hz, 1H), 6.71-6.55 (m, 2H), 5.21 (d, J=53.6 Hz, 1H), 4.97 (s, 2H), 4.59 (dd, J=5.1, 2.6 Hz, 2H), 4.10 (d, J=2.3 Hz, 2H), 3.96 (dd, J=5.4, 2.7 Hz, 2H), 3.24-3.05 (m, 3H), 3.03-2.89 (m, 1H), 2.51-2.38 (m, 3H), 2.33-2.05 (m, 2H), 2.05-1.71 (m, 4H). LCMS: (ESI, m/z): 677.1 [M+H]⁺. Chiral HPLC: Column: CHIRALPAK IC-3, 4.6*50 mm 3 um; Mobile Phase A: Hex (0.1% DEA):EtOH=50:50; Flow rate: 1 mL/min; Injection Volume: 5 ul mL; Retention time: 1.880 min (Second peak).

Example 43: (S)-2-(9-(6-amino-4-methyl-3-(trifluoromethyl)pyridin-2-yl)-8-chloro-4-methyl-5,6-dihydro-4H-[1,4]oxazepino[5,6,7-de]quinazolin-5-yl)acetonitrile

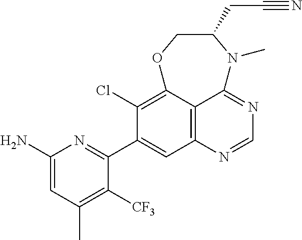 [SMILES: Cc1cc(N)nc(-c2cc3ncnc4c3c(c2Cl)OC[C@H](CC#N)N4C)c1C(F)(F)F]

### Synthetic Route

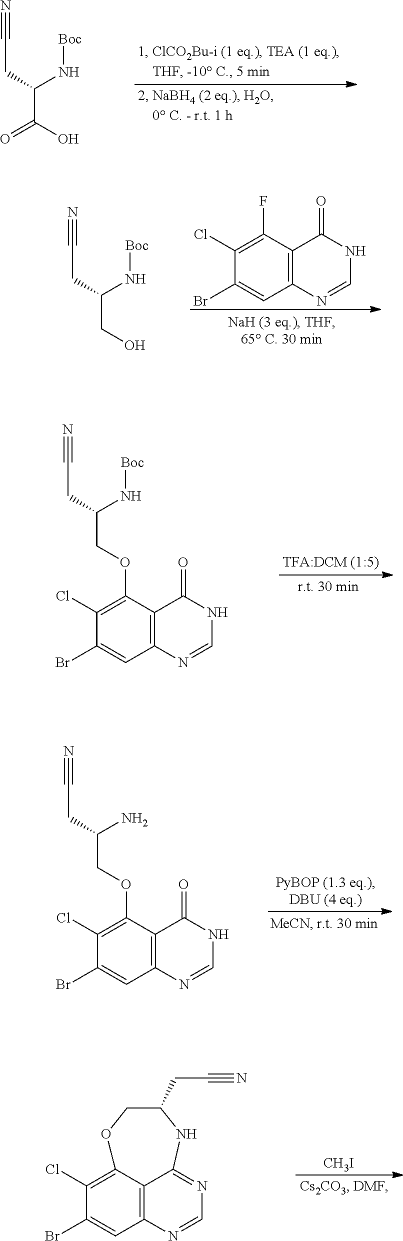

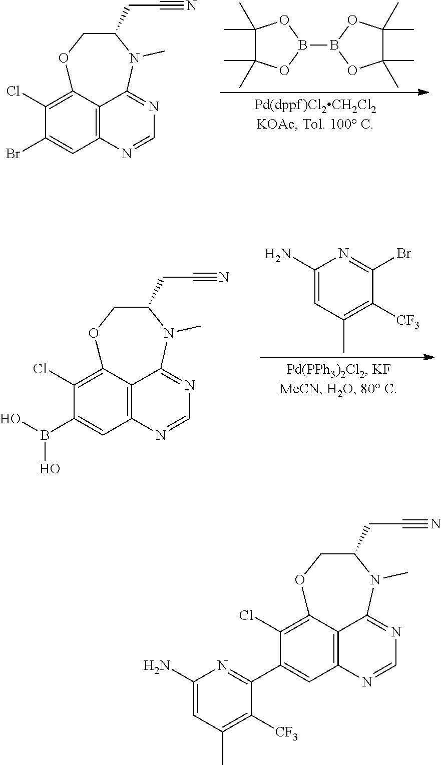

Step 1: tert-butyl (S)-(1-cyano-3-hydroxypropan-2-yl)carbamate

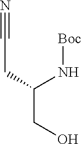 [SMILES: CC(C)(C)OC(=O)N[C@H](CO)CC#N]

A solution of (2S)-2-(tert-butoxycarbonylamino)-3-cyano-propanoic acid (1.00 g, 4.67 mmol) in tetrahydrofuran (5 mL) was treated with triethylamine (0.47 g, 4.67 mmol) at −10° C. and followed by dropwise addition of isobutyl chloroformate (0.67 g, 4.90 mmol). The reaction mixture was stirred for 4 minutes at −10° C. and filtered through a coarse scintered glass funnel. Meanwhile, in another flask, a solution of sodium borohydride (0.36 g, 9.57 mmol) in water (5 mL) was prepared and cooled in an ice water bath. The filtered solution of the mixed anhydride was added dropwise to the cold sodium borohydride solution and the resulting mixture was stirred for 1 hour. The tetrahydrofuran was removed on the rotary evaporator and the reaction was adjusted PH=3.0 with hydrochloric acid (1N) solution and diluted with ethyl acetate and water. The organic layer was washed twice with aqueous sodium bicarbonate solution and dried over anhydrous sodium sulfate. The solvent was removed to afford tert-butyl (S)-(1-cyano-3-hydroxypropan-2-yl)carbamate (0.68 g, 3.40 mmol, 72.8% yield) as a oil. LCMS (ESI, m/z): 201.2 [M+H]⁺.

Step 2: tert-butyl (S)-(1-((7-bromo-6-chloro-4-oxo-3,4-dihydroquinazolin-5-yl)oxy)-3-cyanopropan-2-yl)carbamate

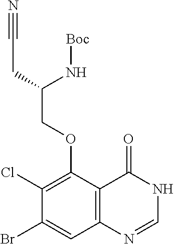 [SMILES: CC(C)(C)OC(=O)N[C@@H](CC#N)COc1c(Cl)c(Br)cc2nc[nH]c(=O)c12]

A solution of tert-butyl (S)-(1-cyano-3-hydroxypropan-2-yl)carbamate (0.72 g, 3.60 mmol) in tetrahydrofuran (5 mL) was added sodium hydride (0.29 g, 7.21 mmol, 60% purity) at 0° C. Then 7-bromo-6-chloro-5-fluoro-3H-quinazolin-4-one (0.50 g, 1.80 mmol) was added and stirred at 0° C. for 5 minutes. Then the solution was heated to 65° C. and stirred for 0.5 hours. After completion, the reaction was quenched by hydrochloric acid (1N) solution. The solvent was concentrated under vacuum. The residue was purified by flash chromatography on reverse phase eluting with water/acetonitrile (0-100%) to afford tert-butyl (S)-(1-((7-bromo-6-chloro-4-oxo-3,4-dihydroquinazolin-5-yl)oxy)-3-cyanopropan-2-yl)carbamate (0.70 g, 1.53 mmol, 84.9% yield) as a white solid. LCMS (ESI, m/z): 357.0 [M+H−100]⁺.

Step 3: (S)-3-amino-4-((7-bromo-6-chloro-4-oxo-3,4-dihydroquinazolin-5-yl)oxy)butanenitrile

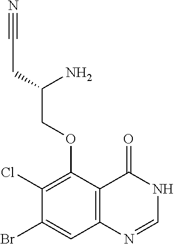 [SMILES: N#CC[C@H](N)COc1c(Cl)c(Br)cc2nc[nH]c(=O)c12]

A solution of tert-butyl (S)-(1-((7-bromo-6-chloro-4-oxo-3,4-dihydroquinazolin-5-yl)oxy)-3-cyanopropan-2-yl)carbamate (0.70 g, 1.53 mmol) in trifluoroacetic acid (1 mL) and dichloromethane (5 mL) was stirred at room temperature for 0.5 hours. After completion, the reaction mixture was adjusted to pH=7 with N,N-diisopropylethylamine. The solvent was concentrated under vacuum. The residue was purified by flash chromatography on silica gel eluting with dichloromethane/methanol (10/1) to afford (S)-3-amino-4-((7-bromo-6-chloro-4-oxo-3,4-dihydroquinazolin-5-yl)oxy)butanenitrile (0.48 mg, 1.34 mmol, 87.8% yield) as a colorless solid. LCMS (ESI, m/z): 357.0 [M+H]⁺.

Step 4: (S)-2-(9-bromo-8-chloro-5,6-dihydro-4H-[1,4]oxazepino[5,6,7-de]quinazolin-5-yl)acetonitrile

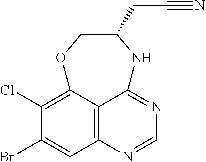 [SMILES: N#CC[C@H]1COc2c(Cl)c(Br)cc3ncnc(c23)N1]

A solution of (3S)-3-amino-4-[(7-bromo-6-chloro-4-oxo-3H-quinazolin-5-yl)oxy]butanenitrile (0.77 g, 2.15 mmol) in acetonitrile (5 mL) was added 1,8-diazabicyclo[5.4.0]undec-7-ene (1.31 g, 8.61 mmol) and benzotriazole-1-yl-oxytripyrrolidinophosphonium hexafluorophosphate (1.46 g, 2.80 mmol) and stirred at room temperature for 30 min. After completion, the solvent was concentrated under vacuum. The residue was purified by flash chromatography on silica gel eluting with dichloromethane/methanol (10/1) to afford (S)-2-(9-bromo-8-chloro-5,6-dihydro-4H-[1,4]oxazepino[5,6,7-de]quinazolin-5-yl)acetonitrile (0.53 g, 1.56 mmol, 72.5% yield) as a white solid. LCMS (ESI, m/z): 339.0 [M+H]⁺.

Step 4: (S)-2-(9-bromo-8-chloro-4-methyl-5,6-dihydro-4H-[1,4]oxazepino[5,6,7-de]quinazolin-5-yl)acetonitrile

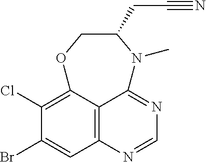 [SMILES: CN1c2ncnc3cc(Br)c(Cl)c(c23)OC[C@@H]1CC#N]

A solution of (S)-2-(9-bromo-8-chloro-5,6-dihydro-4H-[1,4]oxazepino[5,6,7-de]quinazolin-5-yl)acetonitrile (0.40 g, 1.18 mmol), iodomethane (0.20 g, 1.42 mmol) and cesium carbonate (0.77 g, 2.36 mmol) in N,N-dimethylformamide (5 mL) was stirred at 60° C. for 0.5 hours. The reaction mixture was diluted with water, extracted with ethyl acetate and washed with saturated brine. Then the organic layer was dried, filtered and evaporated to afford crude product. The residue was purified by flash chromatography on silica gel eluting with petrol ether/ethyl acetate (1/1) to afford (S)-2-(9-bromo-8-chloro-4-methyl-5,6-dihydro-4H-[1,4]oxazepino[5,6,7-de]quinazolin-5-yl)acetonitrile (0.18 g, 0.51 mmol, 43.2% yield) as a white solid. LCMS (ESI, m/z): 353.0 [M+H]⁺.

Step 4: (S)-(8-chloro-5-(cyanomethyl)-4-methyl-5,6-dihydro-4H-[1,4]oxazepino[5,6,7-de]quinazolin-9-yl)boronic acid

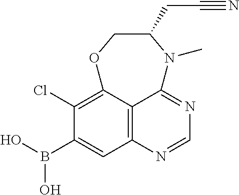 [SMILES: CN1c2ncnc3cc(B(O)O)c(Cl)c(c23)OC[C@@H]1CC#N]

Under nitrogen, a solution of (S)-2-(9-bromo-8-chloro-4-methyl-5,6-dihydro-4H-[1,4]oxazepino[5,6,7-de]quinazolin-5-yl)acetonitrile (0.15 g, 0.42 mmol), bis(pinacolato)diboron (1.08 g, 4.24 mmol), [1,1′-bis(diphenylphosphino)ferrocene]dichloropalladium(II) (0.06 g, 0.08 mmol) and potassium acetate (0.08 g, 1.27 mmol) in toluene (5 mL) was stirred at 100° C. for 3 hours. After completion, the solvent was concentrated under vacuum. The resulting solution was dissolved with dichloromethane. After filtration, the filtrate was concentrated under reduced pressure to afford 700 mg crude of the (S)-(8-chloro-5-(cyanomethyl)-4-methyl-5,6-dihydro-4H-[1,4]oxazepino[5,6,7-de]quinazolin-9-yl)boronic acid as a brown syrup. LCMS (ESI, m/z): 319.0 [M+H]⁺.

Step 4: (S)-2-(9-(6-amino-4-methyl-3-(trifluoromethyl)pyridin-2-yl)-8-chloro-4-methyl-5,6-dihydro-4H-[1,4]oxazepino[5,6,7-de]quinazolin-5-yl)acetonitrile

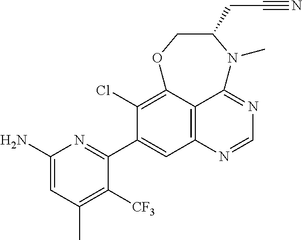 [SMILES: Cc1cc(N)nc(-c2cc3ncnc4c3c(c2Cl)OC[C@H](CC#N)N4C)c1C(F)(F)F]

Under nitrogen, a solution of (S)-(8-chloro-5-(cyanomethyl)-4-methyl-5,6-dihydro-4H-[1,4]oxazepino[5,6,7-de]quinazolin-9-yl)boronic acid (0.70 g, crude), 6-bromo-4-methyl-5-(trifluoromethyl)pyridin-2-amine (0.15 g, 0.57 mmol), bis(triphenylphosphine)palladium(II) chloride (0.06 g, 0.09 mmol) and potassium fluoride (0.08 g, 1.32 mmol) in acetonitrile (10 mL) and water (2 mL) was stirred at 80° C. for 2 hours. The reaction mixture was diluted with dichloromethane and washed with saturated brine and the organic layer was dried over anhydrous sodium sulfate and concentrated under vacuum. The crude product was purified by reverse phase chromatography and Pre-HPLC (Column: XBridge Prep OBD C18 Column, 30×150 mm 5 um; Mobile Phase A: Water (10 MMOL/L NH4HCO3), Mobile Phase B: ACETONITRILE; Flow rate: 60 mL/min; Gradient: 33 B to 43 B in 8 min, 43 B to B in min, B to B in min, B to B in min, B to B in min: 254/220 nm; RT1: 7.55; RT2: Injection Volume: ml; Number Of Runs:) to afford (S)-2-(9-(6-amino-4-methyl-3-(trifluoromethyl)pyridin-2-yl)-8-chloro-4-methyl-5,6-dihydro-4H-[1,4]oxazepino[5,6,7-de]quinazolin-5-yl)acetonitrile (21.8 mg, 0.049 mmol, 11.1% yield). LCMS (ESI, m/z): 449.3 [M+H]⁺.

Example 43: ¹H NMR (400 MHz, Chloroform-d, ppm) δ 8.60 (d, J=2.2 Hz, 1H), 7.47 (d, J=5.8 Hz, 1H), 6.41 (d, J=13.7 Hz, 1H), 4.84 (dt, J=13.3, 3.7 Hz, 1H), 4.45 (dd, J=48.6, 13.2 Hz, 1H), 4.24-4.12 (m, 1H), 3.46 (d, J=4.4 Hz, 3H), 3.03-2.75 (m, 2H), 2.44 (q, J=2.3 Hz, 3H).

Example 44: (S)-6-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

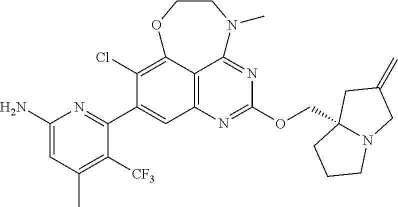 [SMILES: C=C1CN2CCC[C@@]2(COc2nc3c4c(c(Cl)c(-c5nc(N)cc(C)c5C(F)(F)F)cc4n2)OCCN3C)C1]

### Synthetic Route

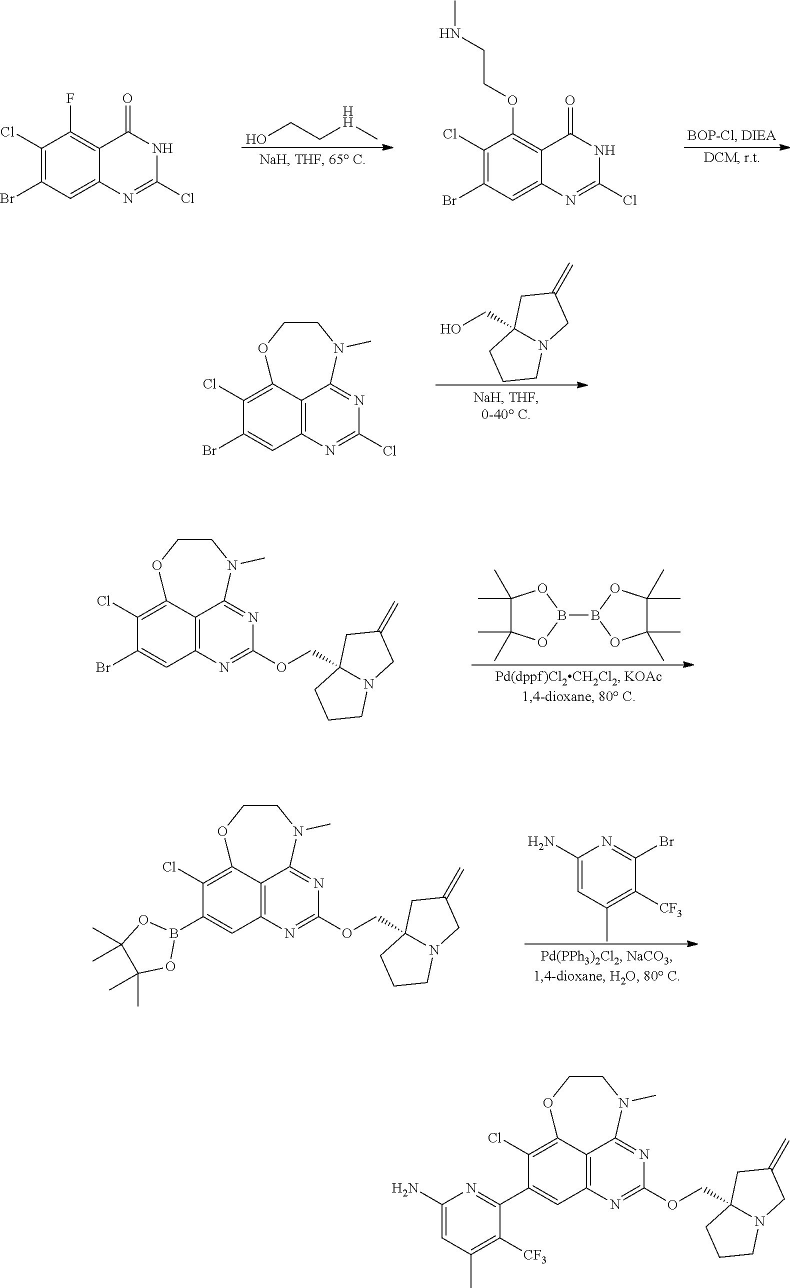

Step 1: 7-bromo-2,6-dichloro-5-(2-(methylamino)ethoxy)quinazolin-4(3H)-one

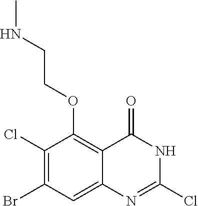 [SMILES: CNCCOc1c(Cl)c(Br)cc2nc(Cl)[nH]c(=O)c12]

A solution of sodium hydride (0.77 g, 19.24 mmol, 60% purity) was added to 2-methylaminoethanol (0.58 g, 7.69 mmol) in tetrahydrofuran (10 mL) cooled to 0° C. 7-bromo-2,6-dichloro-5-fluoro-3H-quinazolin-4-one (2.00 g, 6.41 mmol) was added and stirred at 0° C. for 5 minutes, allowed to warm to room temperature then heated to 65° C. and stirred for 0.5 hours. After completion, the reaction was quenched by ammonium chloride solution. The solvent was concentrated under vacuum. The residue was purified by flash chromatography on silica gel eluting with water/acetonitrile (0-100%) to afford 7-bromo-2,6-dichloro-5-(2-(methylamino)ethoxy)quinazolin-4(3H)-one (1.10 g, 3.00 mmol, 46.7% yield) as a white solid. LCMS (ESI, m/z): 365.95 [M+H]⁺.

Step 2: 9-bromo-2,8-dichloro-4-methyl-5,6-dihydro-4H-[1,4]oxazepino[5,6,7-de]quinazoline

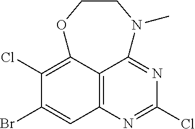 [SMILES: CN1CCOc2c(Cl)c(Br)cc3nc(Cl)nc1c23]

A solution of bis(2-oxo-3-oxazolidinyl)phosphinic chloride (2.29 g, 8.99 mmol) was added to 7-bromo-2,6-dichloro-5-(2-(methylamino)ethoxy)quinazolin-4(3H)-one (1.10 g, 3.00 mmol) and N,N-diisopropylethylamine (5.80 g, 44.96 mmol) in dichloromethane (20 mL) at room temperature and stirred for 12 hours. After completion, the reaction mixture was diluted with dichloromethane, washed with water, dried over anhydrous sodium sulfate and concentrated under vacuum. The residue was purified by reverse phase eluting with water/acetonitrile (0-100%) to afford 9-bromo-2,8-dichloro-4-methyl-5,6-dihydro-4H-[1,4]oxazepino[5,6,7-de]quinazoline (0.45 g, 1.29 mmol, 43% yield) as a white solid. LCMS (ESI, m/z): 348.1 [M+H]⁺.

Step 3: (S)-9-bromo-8-chloro-4-methyl-2-((2-methylenetetrahydro-1H-pyrrolizin-7a(5H)-yl)methoxy)-5,6-dihydro-4H-[1,4]oxazepino[5,6,7-de]quinazoline

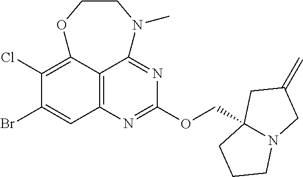 [SMILES: C=C1CN2CCC[C@@]2(COc2nc3c4c(c(Cl)c(Br)cc4n2)OCCN3C)C1]

A solution of (S)-(2-methylenetetrahydro-1H-pyrrolizin-7a(5H)-yl)methanol (2.63 g, 17.19 mmol) and sodium hydride (1.37 g, 34.38 mmol, 60% purity) in tetrahydrofuran (10 mL) was stirred at 40° C. for 5 minutes. Then 9-bromo-2,8-dichloro-4-methyl-5,6-dihydro-4H-[1,4]oxazepino[5,6,7-de]quinazoline (3.00 g, 8.60 mmol) was added and stirred at 40° C. for 2.5 hours. The solvent was concentrated under vacuum. The residue was purified by flash chromatography on silica gel eluting with dichloromethane/methanol (10:1) to afford (S)-9-bromo-8-chloro-4-methyl-2-((2-methylenetetrahydro-1H-pyrrolizin-7a(5H)-yl)methoxy)-5,6-dihydro-4H-[1,4]oxazepino[5,6,7-de]quinazoline (2.12 g, 3.90 mmol, 45.3% yield) as a brown solid. LCMS (ESI, m/z): 465.2 [M+H]⁺.

Step 4: (S)-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)boronic acid

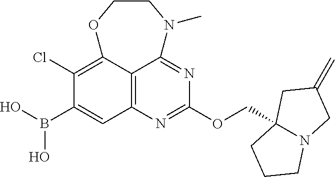 [SMILES: C=C1CN2CCC[C@@]2(COc2nc3c4c(c(Cl)c(B(O)O)cc4n2)OCCN3C)C1]

Under nitrogen, a solution of (S)-9-bromo-8-chloro-4-methyl-2-((2-methylenetetrahydro-1H-pyrrolizin-7a(5H)-yl)methoxy)-5,6-dihydro-4H-[1,4]oxazepino[5,6,7-de]quinazoline (0.40 g, 0.86 mmol), Pin₂B₂ (0.65 g, 2.58 mmol), [1,1′-bis(diphenylphosphino)ferrocene]dichloropalladium(II) (0.14 g, 0.17 mmol) and potassium acetate (0.25 g, 2.58 mmol) in 1,4-dioxane (5 mL) was refluxed at 120° C. for 1 h. The solvent was concentrated under vacuum. The resulting solution was dissolved with dichloromethane. After filtration, the filtrate was concentrated under reduced pressure to afford 1.2 g crude of (S)-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)boronic acid as a brown syrup. LCMS (ESI, m/z): 431.2 [M+H]⁺.

Step 4: (S)-6-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

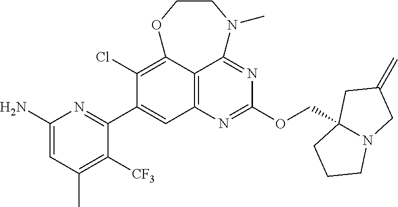 [SMILES: C=C1CN2CCC[C@@]2(COc2nc3c4c(c(Cl)c(-c5nc(N)cc(C)c5C(F)(F)F)cc4n2)OCCN3C)C1]

Under nitrogen, a solution of (S)-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)boronic (1.50 g, crude), 6-bromo-4-methyl-5-(trifluoromethyl)pyridin-2-amine (0.19 g, 0.76 mmol), bis(triphenylphosphine)palladium(11) chloride (0.08 g, 0.12 mmol) and sodium carbonate (0.12 g, 1.17 mmol) in 1,4-dioxane (10 mL) and water (2 mL) was stirred at 80° C. for 10 minutes. After completion, the reaction mixture was diluted with dichloromethane, washed with water, dried over anhydrous sodium sulfate and concentrated under vacuum. The residue was purified by reverse phase with water/acetonitrile and Pre-HPLC (Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 33% B to 63% B in 10 min, 63% B; Wave Length: 254 nm; RT1(min): 8.98; Number Of Runs: 0) to afford (S)-6-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (41.0 mg, 0.072 mmol, 12.2% yield). LCMS (ESI, m/z): 561.2 [M+H]⁺.

Example 44: ¹H NMR (300 MHz, DMSO-d6, ppm) δ 6.90 (s, 1H), 6.71 (s, 2H), 6.44 (s, 1H), 4.90 (s, 2H), 4.76-4.40 (m, 2H), 4.11-3.86 (m, 4H), 3.65-3.51 (m, 1H), 3.31-3.18 (m, 4H), 3.10-2.94 (m, 1H), 2.70-2.54 (m, 2H), 2.35 (s, 4H), 2.05-1.92 (m, 1H), 1.92-1.79 (m, 2H), 1.79-1.59 (m, 1H).

Example 45: (R)-6-(4-((S)-1-(5-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

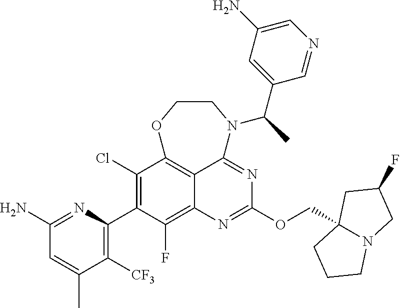

### Synthetic Route

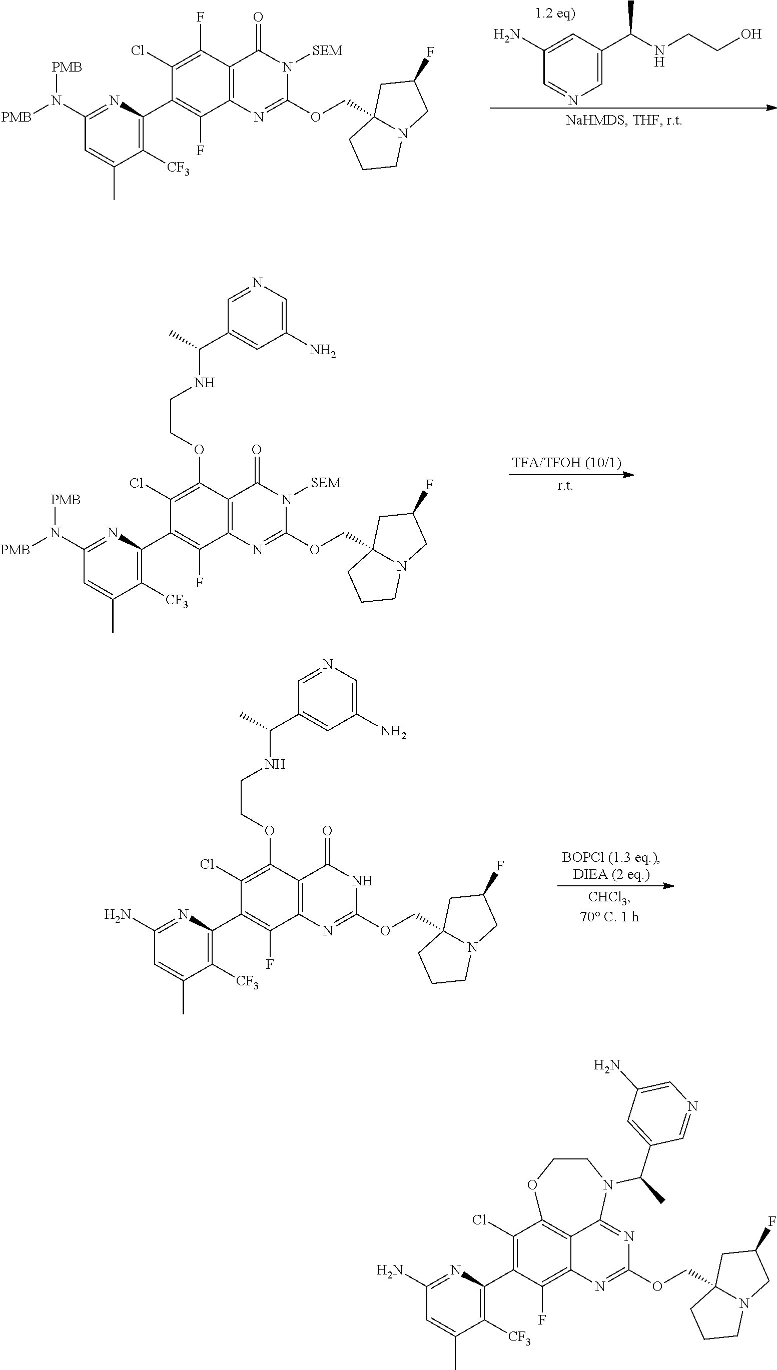

Step 1: 5-(2-(((R)-1-(5-aminopyridin-3-yl)ethyl)amino)ethoxy)-7-((R)-6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one

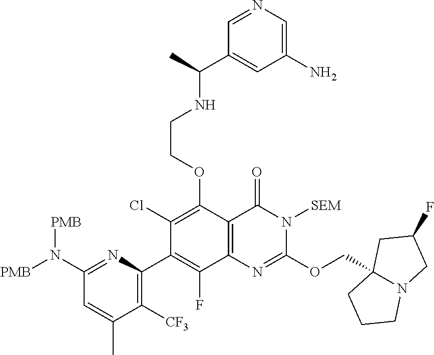

Under nitrogen, 2-[[(1R)-1-(5-amino-3-pyridyl)ethyl]amino]ethanol (0.36 g, 1.98 mmol)) in tetrahydrofuran (80 mL) was added sodium bis(trimethylsilyl)amide (1.67 mL, 1.67 mmol, 1 M in the THF) at 0° C. The reaction was stirred at 0° C. for 5 minutes. The mixture was transferred into a mixture of 7-((R)-6(bis(4-methoxybenzyl)amino)-4-methyl-3-(trifluoromethyl)pyridin-2-yl)-6-chloro-5,8-difluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (1.40 g, 1.52 mmol) in tetrahydrofuran (80 mL) and stirred at room temperature for 6 h. The reaction was quenched by saturated ammonium chloride solution and extracted with dichloromethane. The combined organic layers were washed with saturated sodium chloride and dried over anhydrous sodium sulfate and concentrated under vacuum. The residue was purified by flash chromatography on silica gel eluting with dichloromethane/methanol (20/1) to afford 5-(2-(((R)-1-(5-aminopyridin-3-yl)ethyl)amino)ethoxy)-7-((R)-6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (0.55 g, 0.51 mmol, 33.4% yield) as a colorless solid. LCMS (ESI, m/z): 1079.35 [M+H]⁺.

Step 2: 7-((R)-6-amino-4-methyl-3-(trifluoromethyl)pyridin-2-yl)-5-(2-(((R)-1-(5-aminopyridin-3-yl)ethyl)amino)ethoxy)-6-chloro-8-fluoro-2-(((2R,7aS)-2-fluorotetrahydro-1H-pyrrolizin-7a(5H)-yl)methoxy)quinazolin-4(3H)-one

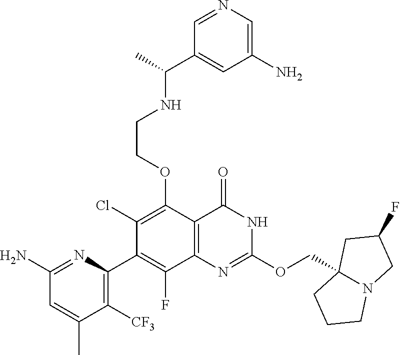

A solution of 5-(2-(((R)-1-(5-aminopyridin-3-yl)ethyl)amino)ethoxy)-7-((R)-6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (0.55 g, 0.51 mmol) in trifluoromethanesulfonic acid (0.5 mL) and trifluoroacetic acid (5 mL) was stirred at room temperature for 10 min. After completion, The solvent was concentrated under vacuum. The residue was purified by Strata-X-C to afford 7-((R)-6-amino-4-methyl-3-(trifluoromethyl)pyridin-2-yl)-5-(2-(((R)-1-(5-aminopyridin-3-yl)ethyl)amino)ethoxy)-6-chloro-8-fluoro-2-(((2R,7aS)-2-fluorotetrahydro-1H-pyrrolizin-7a(5H)-yl)methoxy)quinazolin-4(3H)-one (310 mg, 0.44 mmol, 85.8% yield) as a yellow solid. LCMS (ESI, m/z): 709.2 [M+H]⁺.

Step 3: (R)-6-(4-((R)-1-(5-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

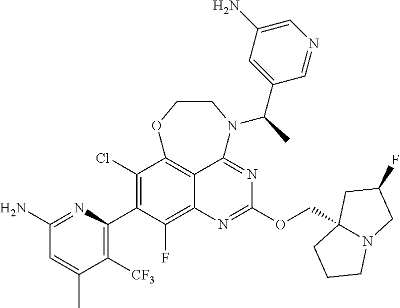

A solution of 7-((R)-6-amino-4-methyl-3-(trifluoromethyl)pyridin-2-yl)-5-(2-(((R)-1-(5-aminopyridin-3-yl)ethyl)amino)ethoxy)-6-chloro-8-fluoro-2-(((2R,7aS)-2-fluorotetrahydro-1H-pyrrolizin-7a(5H)-yl)methoxy)quinazolin-4(3H)-one (0.29 g, 0.41 mmol), bis(2-oxo-3-oxazolidinyl)phosphinic chloride (0.14 g, 0.53 mmol) and N,N-diisopropylethylamine (0.26 g, 2.05 mmol) in chloroform (5 mL) was stirred at 70° C. for 2 hours. The solvent was concentrated under vacuum. The residue was purified by flash chromatography on silica gel eluting with H₂O/acetonitrile and Pre-HPLC (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 36% B to 42% B in 10 min, 42% B; Wave Length: 254/220 nm; RT1(min): 8.67; Number Of Runs: 0) to afford (R)-6-(4-((R)-1-(5-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (84.2 mg, 0.12 mmol, 17.5% yield). LCMS (ESI, m/z): 691.1 [M+H]⁺.

Example 45: ¹H NMR (300 MHz, DMSO-d6, ppm) δ 8.00-7.75 (m, 2H), 7.00-6.72 (m, 3H), 6.51 (s, 1H), 6.49-6.33 (m, 1H), 5.41-5.23 (m, 2H), 5.13 (d, J=53.6 Hz, 1H), 4.60-4.28 (m, 2H), 4.18-4.03 (m, 1H), 4.02-3.92 (m, 1H), 3.80-3.63 (m, 1H), 3.59-3.42 (m, 1H), 3.20-2.95 (m, 3H), 2.90-2.70 (m, 1H), 2.45-2.30 (m, 3H), 2.22-2.12 (m, 1H), 2.08-1.91 (m, 2H), 1.90-1.68 (m, 3H), 1.61 (d, J=6.9 Hz, 3H).

Example 46: 6-(8-chloro-4-(pyridin-3-ylmethyl)-5,6-dihydro-4H-[1,4]oxazepino[5,6,7-de]quinazolin-9-yl)-4-methyl-5-(trifluoromethyl)pyridin-2-amine

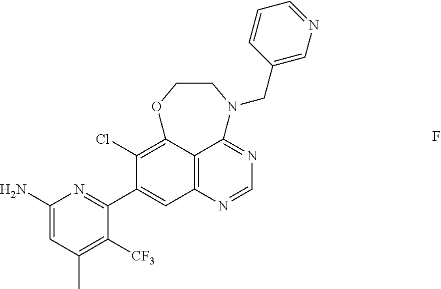

### Synthetic Route

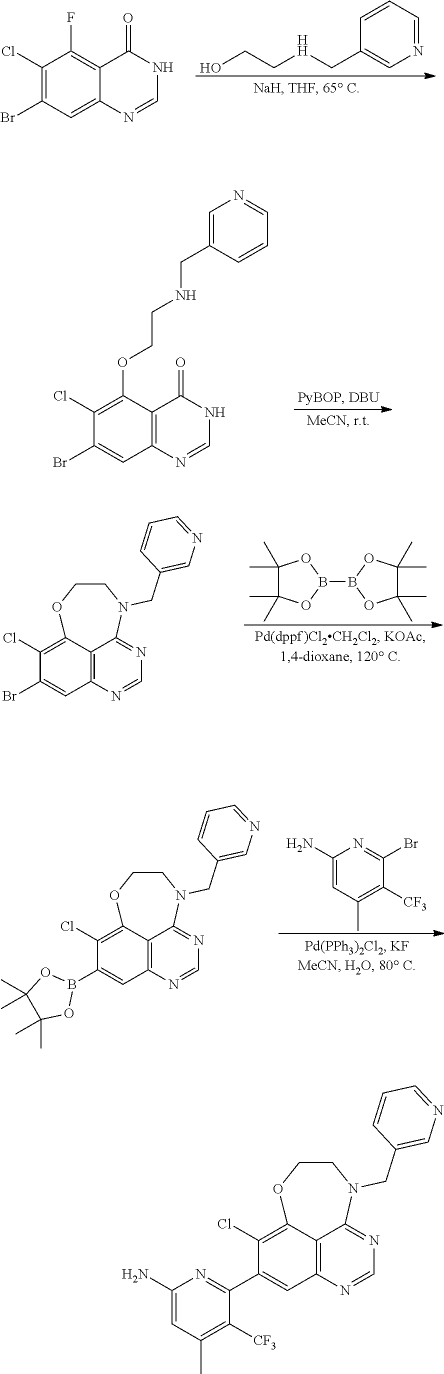

Step 1: 7-bromo-6-chloro-5-(2-((pyridin-3-ylmethyl)amino)ethoxy)quinazolin-4(3H)-one

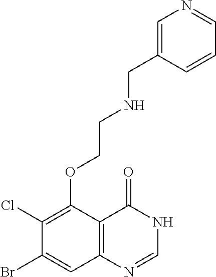 [SMILES: O=c1[nH]cnc2cc(Br)c(Cl)c(OCCNCc3cccnc3)c12]

A solution of sodium hydride (0.46 g, 11.53 mmol, 60% purity) was added to 2-(3-pyridylmethylamino)ethanol (0.53 g, 3.46 mmol) in tetrahydrofuran (10 mL) cooled to 0° C. 7-bromo-6-chloro-5-fluoro-3H-quinazolin-4-one (1.00 g, 2.88 mmol) was added and stirred at 0° C. for 5 minutes, allowed to warm to room temperature then heated to 65° C. and stirred for 2 hours. After completion, the reaction was quenched by ammonium chloride solution. The solvent was concentrated under vacuum. The residue was purified by flash chromatography on silica gel eluting with water/acetonitrile (0-100%) to afford 7-bromo-6-chloro-5-(2-((pyridin-3-ylmethyl)amino)ethoxy)quinazolin-4(3H)-one (700 mg, 1.71 mmol, 59.3% yield) as a white solid. LCMS (ESI, m/z): 409.1 [M+H]⁺.

Step 2: 9-bromo-8-chloro-4-(pyridin-3-ylmethyl)-5,6-dihydro-4H-[1,4]oxazepino[5,6,7-de]quinazoline

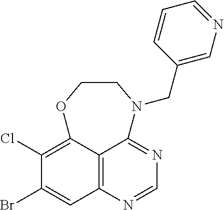 [SMILES: Clc1c(Br)cc2ncnc3c2c1OCCN3Cc1cccnc1]

A solution of benzotriazole-1-yl-oxytripyrrolidinophosphonium hexafluorophosphate (2.58 g, 4.96 mmol) was added to 7-bromo-6-chloro-5-(2-((pyridin-3-ylmethyl)amino)ethoxy)quinazolin-4(3H)-one (1.35 g, 3.3 mmol) and 1,8-diazabicyclo[5.4.0]undec-7-ene (1.51 g, 9.91 mmol) in acetonitrile (10 mL) at room temperature and stirred for 2 hours. After completion, the solvent was concentrated under vacuum. The residue was purified by flash chromatography on silica gel eluting with water/acetonitrile (0-100%) to afford 9-bromo-8-chloro-4-(pyridin-3-ylmethyl)-5,6-dihydro-4H-[1,4]oxazepino[5,6,7-de]quinazoline (1.10 g, 2.81 mmol, 85% yield) as a white solid. LCMS (ESI, m/z): 391.1 [M+H]⁺.

Step 3: (8-chloro-4-(pyridin-3-ylmethyl)-5,6-dihydro-4H-[1,4]oxazepino[5,6,7-de]quinazolin-9-yl)boronic acid

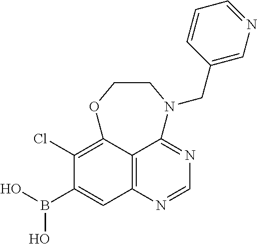 [SMILES: OB(O)c1cc2ncnc3c2c(c1Cl)OCCN3Cc1cccnc1]

Under nitrogen, a solution of 9-bromo-8-chloro-4-(pyridin-3-ylmethyl)-5,6-dihydro-4H-[1,4]oxazepino[5,6,7-de]quinazoline (1.10 g, 2.81 mmol), bis(pinacolato)diboron (1.43 g, 5.62 mmol), [1,1-bis(diphenylphosphino)ferrocene]dichloropalladium(II) (0.46 g, 0.56 mmol) and potassium acetate (0.83 g, 8.43 mmol) in 1,4-dioxane (8 mL) was refluxed for 16 hours. After completion, the solvent was concentrated under vacuum. The resulting solution was dissolved with dichloromethane. After filtration, the filtrate was concentrated under reduced pressure to afford 1.50 g crude of (8-chloro-4-(pyridin-3-ylmethyl)-5,6-dihydro-4H-[1,4]oxazepino[5,6,7-de]quinazolin-9-yl)boronic acid as a brown syrup. LCMS (ESI, m/z): 357.0 [M+H]⁺.

Step 4: 6-(8-chloro-4-(pyridin-3-ylmethyl)-5,6-dihydro-4H-[1,4]oxazepino[5,6,7-de]quinazolin-9-yl)-4-methyl-5-(trifluoromethyl)pyridin-2-amine

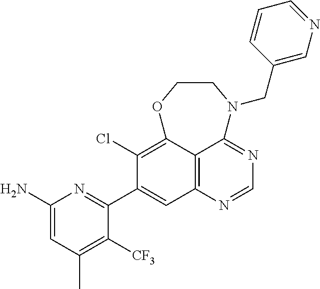 [SMILES: Cc1cc(N)nc(-c2cc3ncnc4c3c(c2Cl)OCCN4Cc2cccnc2)c1C(F)(F)F]

Under nitrogen, a solution of (8-chloro-4-(pyridin-3-ylmethyl)-5,6-dihydro-4H-[1,4]oxazepino[5,6,7-de]quinazolin-9-yl)boronic acid (1.50 g, crude), 6-bromo-4-methyl-5-(trifluoromethyl)pyridin-2-amine (0.28 g, 1.11 mmol), bis(triphenylphosphine)palladium(II) chloride (0.12 g, 0.17 mmol) and potassium fluoride (0.15 g, 2.57 mmol) in acetonitrile (10 mL) and water (2 mL) was stirred at 80° C. for 2 hours. After completion, the reaction mixture was diluted with dichloromethane, washed with water and the organic layers were combined. The organic layer was dried over anhydrous sodium sulfate and concentrated under vacuum. The residue was purified by flash chromatography on silica gel eluting with dichloromethane/methanol (5/1) and H₂O-acetonitrile and Pre-HPLC (Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 25% B to 55% B in 10 min, 55% B; Wave Length: 254 nm; RT1(min): 9.05; Number Of Runs: 0) to afford 6-(8-chloro-4-(pyridin-3-ylmethyl)-5,6-dihydro-4H-[1,4]oxazepino[5,6,7-de]quinazolin-9-yl)-4-methyl-5-(trifluoromethyl)pyridin-2-amine (102.9 mg, 0.21 mmol, 24.7% yield). LCMS (ESI, m/z): 487.3 [M+H]⁺.

Example 46: ¹H NMR (300 MHz, DMSO-d6, ppm) δ 8.61 (d, J=1.2 Hz, 1H), 8.47 (d, J=3.6 Hz, 1H), 8.43 (s, 1H), 7.85-7.75 (m, 1H), 7.43-7.30 (m, 1H), 7.20 (s, 1H), 6.76 (s, 2H), 6.46 (s, 1H), 5.28-5.02 (m, 2H), 4.78-4.53 (m, 2H), 4.06-3.88 (m, 2H), 2.38-2.30 (m, 3H).

Example 47a&47b: 6-((R)-4-((1H-imidazol-5-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((1H-imidazol-5-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

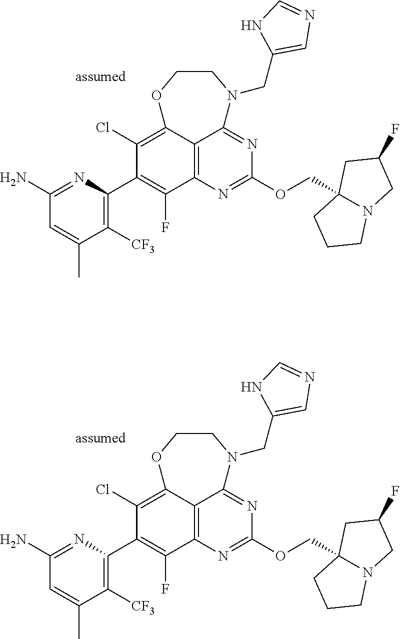

### Synthetic Route

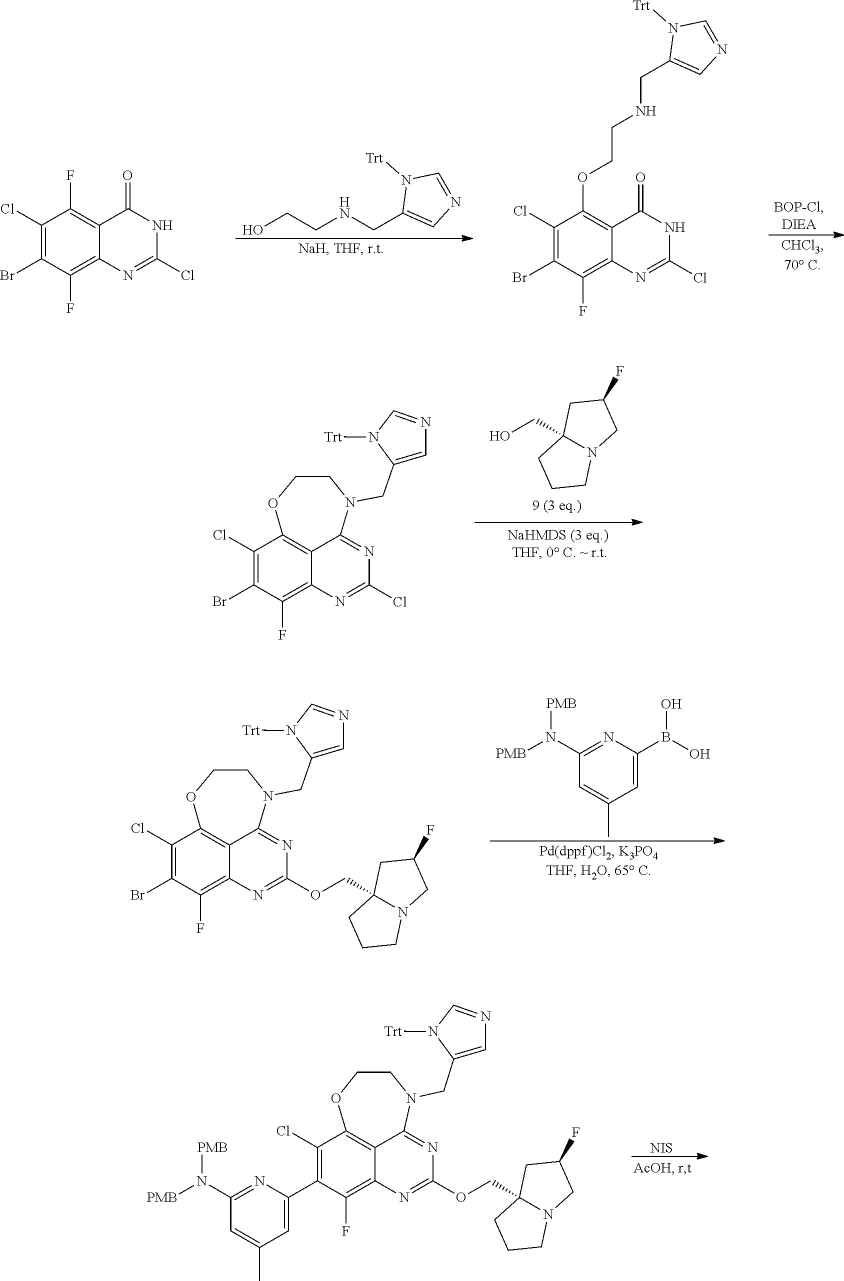

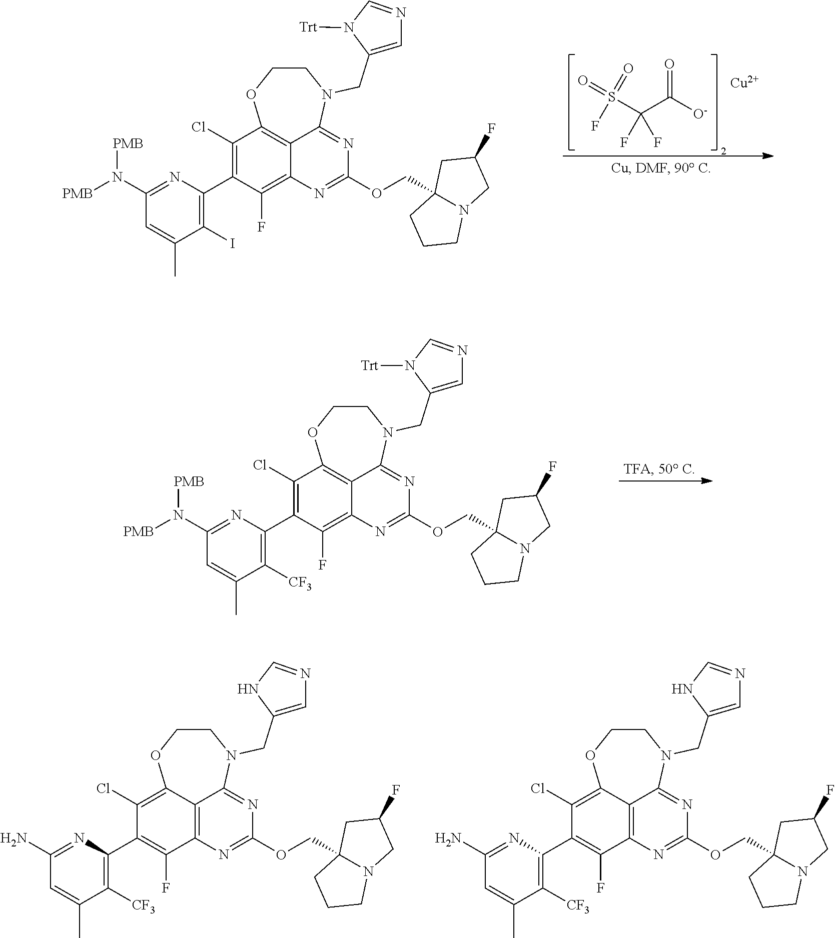

Step 1: 7-bromo-2,6-dichloro-8-fluoro-5-(2-(((1-trityl-1H-imidazol-5-yl)methyl)amino)ethoxy)quinazolin-4(3H)-one

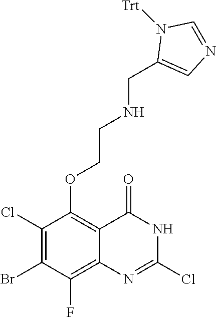

A solution of sodium hydride (0.58 g, 14.55 mmol, 60% purity) was added to 2-(((1-trityl-1H-imidazol-5-yl)methyl)amino)ethan-1-ol (1.02 g, 2.67 mmol) in tetrahydrofuran (10 mL) cooled to 0° C. Then 7-bromo-2,6-dichloro-5,8-difluoro-3H-quinazolin-4-one (0.80 g, 2.42 mmol) was added and stirred at 0° C. for 5 minutes, allowed to warm to room temperature then heated to room temperature and stirred for 2 hours. After completion, the reaction was quenched by ammonium chloride solution. The solvent was concentrated under vacuum. The residue was purified by flash chromatography on silica gel eluting with dichloromethane/methanol (10/1) to afford 7-bromo-2,6-dichloro-8-fluoro-5-(2-(((1-trityl-1H-imidazol-5-yl)methyl)amino)ethoxy)quinazolin-4(3H)-one (0.90 g, 1.30 mmol, 53.5% yield) as a white solid. LCMS (ESI, m/z): 692.0 [M+H]⁺.

Step 2: 9-bromo-2,8-dichloro-10-fluoro-4-((1-trityl-1H-imidazol-5-yl)methyl)-5,6-dihydro-4H-[1,4]oxazepino[5,6,7-de]quinazoline

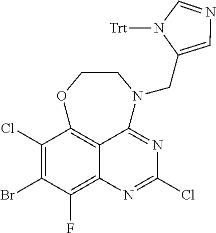

A solution of bis(2-oxo-3-oxazolidinyl)phosphinic chloride (0.43 g, 1.69 mmol) was added to 7-bromo-2,6-dichloro-8-fluoro-5-(2-(((1-trityl-1H-imidazol-5-yl)methyl)amino)ethoxy)quinazolin-4(3H)-one (0.90 g, 1.30 mmol) and N,N-diisopropylethylamine (0.33 g, 2.60 mmol) in chloroform (5 mL) at 70° C. and stirred for 2 hours. The reaction mixture was diluted with dichloromethane, washed with water, dried over anhydrous sodium sulfate and concentrated under vacuum. The residue was purified by flash chromatography on silica gel eluting with dichloromethane/methanol (20/1) to afford 9-bromo-2,8-dichloro-10-fluoro-4-((1-trityl-1H-imidazol-5-yl)methyl)-5,6-dihydro-4H-[1,4]oxazepino[5,6,7-de]quinazoline (0.48 g, 0.71 mmol, 54.8% yield) as a white solid. LCMS (ESI, m/z): 674.0 [M+H]⁺.

Step 3: 9-bromo-8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazoline

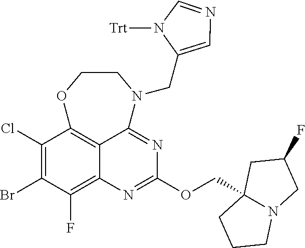

Under nitrogen, sodium bis(trimethylsilyl)amide (2.22 mL, 2.22 mmol, 1M in THF) was added to ((2R,7aS)-2-fluorotetrahydro-1H-pyrrolizin-7a(5H)-yl)methanol (0.35 g, 2.22 mmol) in tetrahydrofuran (5 mL) cooled to 0° C. Then 9-bromo-2,8-dichloro-10-fluoro-4-((1-trityl-1H-imidazol-5-yl)methyl)-5,6-dihydro-4H-[1,4]oxazepino[5,6,7-de]quinazoline (0.50 g, 0.74 mmol) was added and stirred at 0° C. for 5 minutes, allowed to warm to room temperature and stirred for 12 hours. After completion, the reaction mixture was diluted with 1N hydrochloric acid solution, extracted with dichloromethane, washed with water and the organic layers were combined. The organic layer was dried over anhydrous sodium sulfate and concentrated under vacuum. The residue was purified by flash chromatography on silica gel eluting with dichloromethane/methanol (10/1) to afford 9-bromo-8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazoline (380.0 mg, 0.48 mmol, 64.3% yield) as a colorless solid. LCMS (ESI, m/z): 797.2 [M+H]⁺.

Step 4: 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-N,N-bis(4-methoxybenzyl)-4-methylpyridin-2-amine

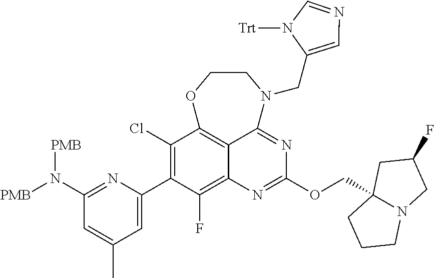

Under nitrogen, a solution of 9-bromo-8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazoline (0.63 g, 0.79 mmol), [6-[bis[(4-methoxyphenyl)methyl]amino]-4-methyl-2-pyridyl]boronic acid (1.03 g, 1.58 mmol), 1,1′-bis(diphenylphosphino)ferrocene-palladium(II)dichloride dichloromethane complex (0.06 g, 0.08 mmol) and potassium phosphate (0.50 g, 2.37 mmol) in tetrahydrofuran (3 mL) and Water (0.6 mL) was stirred for 60 minutes at 65° C. After completion, the reaction mixture was diluted with dichloromethane, washed with water, dried over anhydrous sodium sulfate and concentrated under vacuum. The residue was purified by flash chromatography on silica gel eluting with dichloromethane/methanol (10/1) to afford 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-N,N-bis(4-methoxybenzyl)-4-methylpyridin-2-amine (0.60 g, 0.56 mmol, 71.3% yield) as a yellow solid. LCMS (ESI, m/z): 1065.3 [M+H]⁺.

Step 5: 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-5-iodo-N,N-bis(4-methoxybenzyl)-4-methylpyridin-2-amine

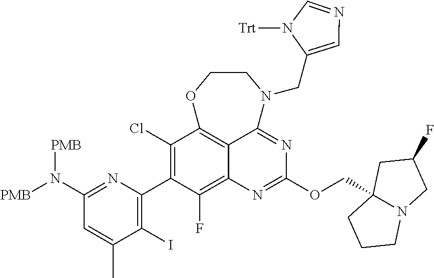

A solution of 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-N,N-bis(4-methoxybenzyl)-4-methylpyridin-2-amine (0.59 g, 0.55 mmol) and N-iodosuccinimide (0.14 g, 0.61 mmol) in acetic acid (10 mL) was stirred at room temperature for 0.5 hours. After completion, the reaction was quenched with saturated sodium thiosulfate solution. The reaction mixture was diluted with ethyl acetate, washed with water, dried over anhydrous sodium sulfate and concentrated under vacuum. The residue was purified by flash chromatography on silica gel eluting with dichloromethane/methanol (20/1) to afford 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-5-iodo-N,N-bis(4-methoxybenzyl)-4-methylpyridin-2-amine (0.45 g, 0.38 mmol, 68.2% yield) as a yellow solid. LCMS (ESI, m/z): 1191.5 [M+H]⁺.

Step 6: 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

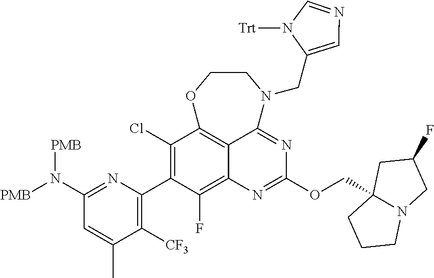

Under nitrogen, a solution of 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-5-iodo-N,N-bis(4-methoxybenzyl)-4-methylpyridin-2-amine (430 mg, 0.36 mmol) and Copper (687.97 mg, 10.83 mmol) in N,N-dimethylformamide (10 mL) was stirred 5 mins at 0° C. Then bis[(2,2-difluoro-2-fluorosulfonyl-acetyl)oxy]copper (4.52 g, 10.83 mmol) was added and stirred at 0° C. for 10 minutes. Then the solution was warmed at room temperature and then under 90° C. and stirred for 10 minutes. After completion, the resulting reaction mixture was filtered, the filtrate was diluted with ethyl acetate, washed with water, dried over anhydrous sodium sulfate and concentrated under vacuum. The residue was purified by flash chromatography on silica gel eluting with dichloromethane/methanol (20/1) to afford 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (230.0 mg, 0.15 mmol, 42.2% yield) as a yellow solid. LCMS (ESI, m/z): 1133.45 [M+H]⁺.

Step 7: 6-((R)-4-((1H-imidazol-5-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((1H-imidazol-5-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

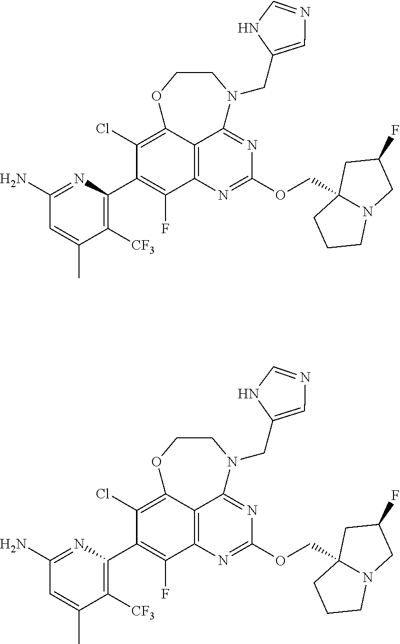

A solution of 6-(8-chloro-10-fluoro-2-(((2R,7aS)-2-fluorotetrahydro-1H-pyrrolizin-7a(5H)-yl)methoxy)-4-((1-trityl-1H-imidazol-5-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (290 mg, 0.20 mmol) in trifluoroacetic acid (5 mL) was stirred at 50° C. for 10 hours. After completion, the solvent was concentrated under vacuum. The residue was purified by flash chromatography on silica gel eluting with water/acetonitrile, Pre-HPLC (Column: XBridge Prep OBD C18 Column, 30*150 mm, 5 μm; Mobile Phase A: Water (10 MMOL/L NH4HCO3), Mobile Phase B: ACN; Flow rate: 60 mL/min; Gradient: 27% B to 57% B in 9 min; Wave Length: 254 nm; RT1(min): 8.5; Number Of Runs: 0) and CHIRAL_HPLC (Column: CHIRAL ART Cellulose-SB, 2*25 cm, 5 μm; Mobile Phase A: Hex (0.5% 2M NH3-MeOH)-HPLC, Mobile Phase B: EtOH-HPLC; Flow rate: 20 mL/min; Gradient: 50% B to 50% B in 19 min; Wave Length: 254/220 nm; RT1(min): 4.714; RT2(min): 13.597; Sample Solvent: EtOH-HPLC; Injection Volume: 2 mL; 1) to afford 6-((R)-4-((1H-imidazol-5-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (10.9 mg, 0.017 mmol, 8.2% yield) and 6-((S)-4-((1H-imidazol-5-yl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (11.2 mg, 0.017 mmol, 8.4% yield). The stereo chemistry of title compounds was arbitrarily assigned

Example 47a: ¹H NMR (300 MHz, Methanol-d4, ppm) δ 7.68 (s, 1H), 7.16 (s, 1H), 6.60 (s, 1H), 5.31 (d, J=53.6 Hz, 1H), 5.10 (s, 2H), 4.62-4.49 (m, 2H), 4.35-4.16 (m, 2H), 3.99 (d, J=3.6 Hz, 2H), 3.27-3.21 (m, 2H), 3.20-3.14 (m, 1H), 3.08-2.94 (m, 1H), 2.45 (d, J=1.5 Hz, 3H), 2.40-2.01 (m, 3H), 2.01-1.78 (m, 3H). LCMS (ESI, m/z): 651.2 [M+H]⁺. Chiral HPLC: Column: CHIRAL ART Cellulose-SB, 4.6*100 mm, 3 μm; Mobile Phase A: Hex (0.1% DEA):EtOH=50:50; Flow rate: 1 mL/min; Injection Volume: 5 ul mL; Retention time: 2.140 min (First peak).

Example 47 b: ¹H NMR (300 MHz, Methanol-d4, ppm) δ 7.68 (s, 1H), 7.16 (s, 1H), 6.60 (s, 1H), 5.31 (d, J=53.6 Hz, 1H), 5.10 (s, 2H), 4.59-4.50 (m, 2H), 4.33-4.18 (m, 2H), 4.04-3.96 (m, 2H), 3.30-3.20 (m, 2H), 3.20-3.16 (m, 1H), 3.11-2.95 (m, 1H), 2.50-2.40 (m, 3H), 2.40-2.05 (m, 3H), 2.05-1.85 (m, 3H). LCMS (ESI, m/z): 651.2 [M+H]⁺. Chiral HPLC: Column: CHIRAL ART Cellulose-SB, 4.6*100 mm, 3 μm; Mobile Phase A: Hex (0.1% DEA):EtOH=50:50; Flow rate: 1 mL/min; Injection Volume: 5 ul mL; Retention time: 6.450 min (Second peak).

Example 48: 6-(4-((R)-1-(2-aminopyridin-3-yl)ethyl)-8-chloro-10-fluoro-2-(((2R,7aS)-2-fluoro-hexahydro-1H-pyrrolizin-7a-yl)methoxy)-5,6-dihydro-4H-[1,4]oxazepino[5,6,7-de]quinazolin-9-yl)-4-methyl-5-(trifluoromethyl)pyridin-2-amine

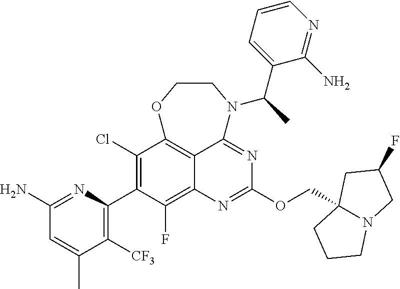

### Synthetic Route

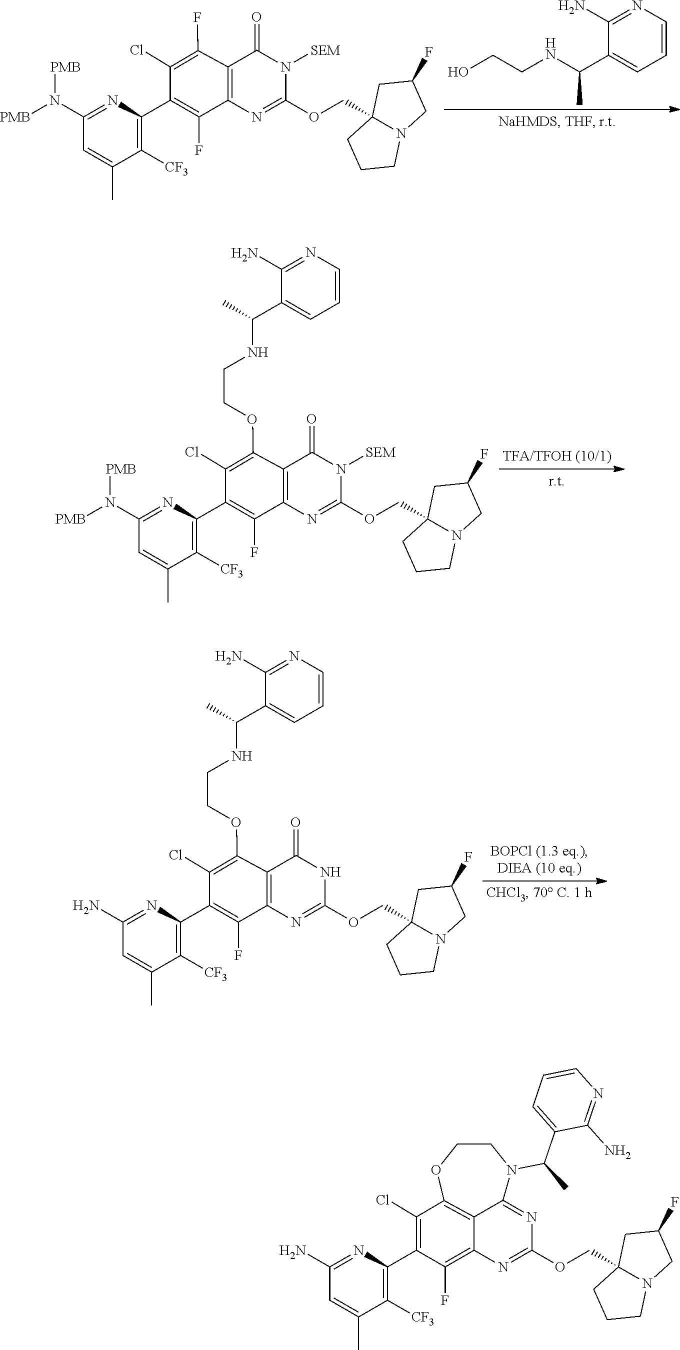

Step 1: 5-(2-((R)-1-(2-aminopyridin-3-yl)ethylamino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluoro-hexahydro-1H-pyrrolizin-7a-yl)methoxy)-3-((2-(trimethylsilyl)ethoxy)methyl)quinazolin-4(3H)-one

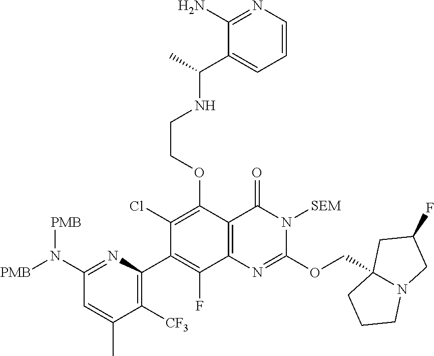

Under nitrogen, 2-[[(1R)-1-(2-amino-3-pyridyl)ethyl]amino]ethanol (0.39 g, 2.12 mmol)) in tetrahydrofuran (70 mL) was added sodium bis(trimethylsilyl)amide (1 M in the THF) (1.90 mL, 1.90 mmol) at 0° C. and stirred at 0° C. for 5 min. The mixture was transferred into a mixture of 7-((R)-6-(bis(4-methoxybenzyl)amino)-4-methyl-3-(trifluoromethyl)pyridin-2-yl)-6-chloro-5,8-difluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (1.50 g, 1.63 mmol) in tetrahydrofuran (70 mL) and stirred at room temperature for 5 h. The reaction was quenched by saturated ammonium chloride solution and extracted with dichloromethane. The combined organic layers dried over anhydrous sodium sulfate and concentrated under vacuum. The residue was purified by flash chromatography on silica gel eluting with dichloromethane/methanol (20/1) to afford 5-(2-((R)-1-(2-aminopyridin-3-yl)ethylamino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluoro-hexahydro-1H-pyrrolizin-7a-yl)methoxy)-3-((2-(trimethylsilyl)ethoxy)methyl)quinazolin-4(3H)-one (0.60 g, 0.47 mmol, 28.9% yield) as a colorless solid. LCMS (ESI, m/z): 1079.4 [M+H]⁺.

Step 2: 7-(6-amino-4-methyl-3-(trifluoromethyl)pyridin-2-yl)-5-(2-((R)-1-(2-aminopyridin-3-yl)ethylamino)ethoxy)-6-chloro-8-fluoro-2-(((2R,7aS)-2-fluoro-hexahydro-1H-pyrrolizin-7a-yl)methoxy)quinazolin-4(3H)-one

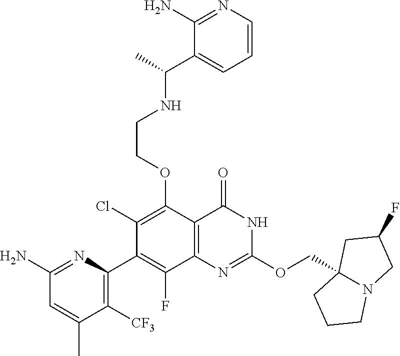

A solution of 5-(2-((R)-1-(2-aminopyridin-3-yl)ethylamino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluoro-hexahydro-1H-pyrrolizin-7a-yl)methoxy)-3-((2-(trimethylsilyl)ethoxy)methyl)quinazolin-4(3H)-one (0.60 g, 0.47 mmol) in trifluoromethanesulfonic (0.5 mL) and 2,2,2-trifluoroacetic acid (5 mL) was stirred at room temperature for 10 min. The solvent was concentrated under vacuum. The residue was purified by Strata-X-C column to afford 7-(6-amino-4-methyl-3-(trifluoromethyl)pyridin-2-yl)-5-(2-((R)-1-(2-aminopyridin-3-yl)ethylamino)ethoxy)-6-chloro-8-fluoro-2-(((2R,7aS)-2-fluoro-hexahydro-1H-pyrrolizin-7a-yl)methoxy)quinazolin-4(3H)-one (380.0 mg, 0.46 mmol, 96.4% yield) as a yellow solid. LCMS (ESI, m/z): 709.4 [M+H]⁺.

Step 3: 6-(4-((R)-1-(2-aminopyridin-3-yl)ethyl)-8-chloro-10-fluoro-2-(((2R,7aS)-2-fluoro-hexahydro-1H-pyrrolizin-7a-yl)methoxy)-5,6-dihydro-4H-[1,4]oxazepino[5,6,7-de]quinazolin-9-yl)-4-methyl-5-(trifluoromethyl)pyridin-2-amine

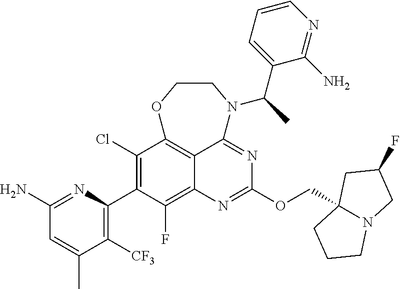

A solution of 7-(6-amino-4-methyl-3-(trifluoromethyl)pyridin-2-yl)-5-(2-((R)-1-(2-aminopyridin-3-yl)ethylamino)ethoxy)-6-chloro-8-fluoro-2-(((2R,7aS)-2-fluoro-hexahydro-1H-pyrrolizin-7a-yl)methoxy)quinazolin-4(3H)-one (0.38 g, 0.46 mmol), bis(2-oxo-3-oxazolidinyl)phosphinic chloride (0.15 g, 0.59 mmol) and N,N-diisopropylethylamine (0.59 g, 4.56 mmol) in chloroform (10 mL) was stirred at 70° C. for 5 hours. The solvent was concentrated under vacuum. The residue was purified by flash chromatography on reverse phase eluting with H₂O/acetonitrile (0-100) and Prep-HPLC (Column: Xselect CSH F-Phenyl OBD column, 19*250 mm, 5 μm; Mobile Phase A: Water (10 mmol/L NH4HCO3), Mobile Phase B: MeOH—Preparative; Flow rate: 25 mL/min; Gradient: 67% B to 85% B in 8 min, 85% B; Wave Length: 254 nm; RT1(min): 6.58; Number Of Runs: 0) to afford 6-(4-((R)-1-(2-aminopyridin-3-yl)ethyl)-8-chloro-10-fluoro-2-(((2R,7aS)-2-fluoro-hexahydro-1H-pyrrolizin-7a-yl)methoxy)-5,6-dihydro-4H-[1,4]oxazepino[5,6,7-de]quinazolin-9-yl)-4-methyl-5-(trifluoromethyl)pyridin-2-amine (68.1 mg, 0.099 mmol, 18.4% yield). LCMS (ESI, m/z): 691.25 [M+H]⁺.

Example 48: ¹H NMR (400 MHz, Methanol-d4, ppm) δ 7.94 (s, 1H), 7.75 (d, J=7.6 Hz, 1H), 6.77 (dd, J=7.5, 5.1 Hz, 1H), 6.60 (s, 1H), 6.53 (q, J=6.8 Hz, 1H), 5.28 (d, J=53.6 Hz, 1H), 5.51-5.35 (m, 1H), 5.35-5.20 (m, 3H), 3.80-3.43 (m, 2H), 3.26-3.09 (m, 3H), 3.09-2.90 (m, 1H), 2.43 (d, J=0.4 Hz, 3H), 2.30-2.06 (m, 3H), 2.06-1.80 (m, 3H), 1.75-1.55 (m, 3H).

Example 49: 6-(4-((2-aminopyridin-3-yl)methyl)-8-chloro-2-(((2S,4R)-4-fluoro-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

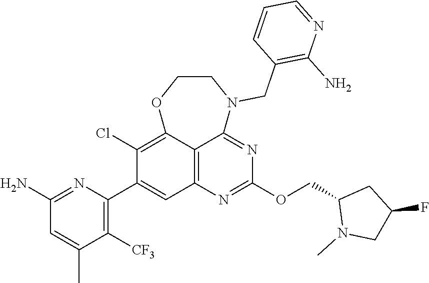 [SMILES: Cc1cc(N)nc(-c2cc3nc(OC[C@@H]4C[C@@H](F)CN4C)nc4c3c(c2Cl)OCCN4Cc2cccnc2N)c1C(F)(F)F]

### Synthetic Route

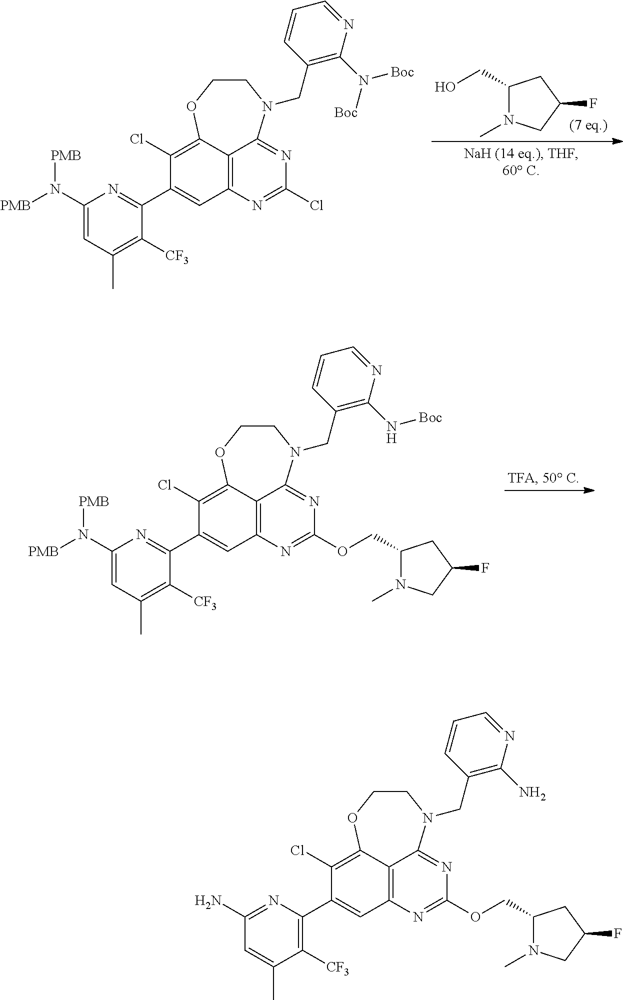

Step 1: tert-butyl 3-((9-(6-(bis(4-methoxybenzyl)amino)-4-methyl-3-(trifluoromethyl)pyridin-2-yl)-8-chloro-2-(((2S,4R)-4-fluoro-1-methylpyrrolidin-2-yl)methoxy)-5,6-dihydro-[1,4]oxazepino[5,6,7-de]quinazolin-4-yl)methyl)pyridin-2-ylcarbamate

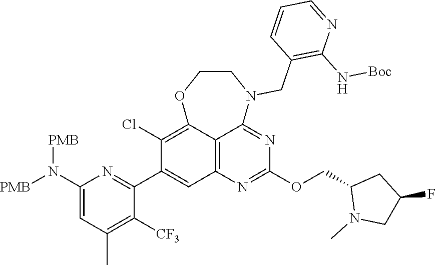

A solution of ((2S,4R)-4-fluoro-1-methylpyrrolidin-2-yl)methanol (163.3 mg, 0.92 mmol) and sodium hydride (73.7 mg, 1.84 mmol, 60% purity) in tetrahydrofuran (9 mL) was stirred at 0° C. for 15 min. Then tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.05,14]tetradeca-1,3,5(14),6,8-pentaen-13-yl]methyl]-2-pyridyl]-N-tert-butoxycarbonyl-carbamate (90.0 mg, 0.09 mmol) was added and stirred at 60° C. for 1 hour. The solvent was quenched with saturated ammonium chloride solution. The reaction mixture was diluted with dichloromethane. The resulting solution was washed with water. The organic layer was dried over anhydrous sodium sulfate and concentrated under vacuum. The residue was purified by flash chromatography on silica gel eluting with dichloromethane/methanol (20/1) to afford tert-butyl 3-((9-(6-(bis(4-methoxybenzyl)amino)-4-methyl-3-(trifluoromethyl)pyridin-2-yl)-8-chloro-2-(((2S,4R)-4-fluoro-1-methylpyrrolidin-2-yl)methoxy)-5,6-dihydro-[1,4]oxazepino[5,6,7-de]quinazolin-4-yl)methyl)pyridin-2-ylcarbamate (100.0 mg, 0.09 mmol, 96% yield) as a white solid. LCMS (ESI, m/z): 1017.5 [M+H]⁺.

Step 2: 6-(4-((2-aminopyridin-3-yl)methyl)-8-chloro-2-(((2S,4R)-4-fluoro-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

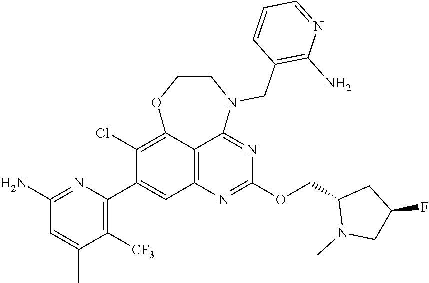 [SMILES: Cc1cc(N)nc(-c2cc3nc(OC[C@@H]4C[C@@H](F)CN4C)nc4c3c(c2Cl)OCCN4Cc2cccnc2N)c1C(F)(F)F]

A solution of tert-butyl 3-((9-(6-(bis(4-methoxybenzyl)amino)-4-methyl-3-(trifluoromethyl)pyridin-2-yl)-8-chloro-2-(((2S,4R)-4-fluoro-1-methylpyrrolidin-2-yl)methoxy)-5,6-dihydro-[1,4]oxazepino[5,6,7-de]quinazolin-4-yl)methyl)pyridin-2-ylcarbamate (60.0 mg, 0.06 mmol) in 2,2,2-trifluoroacetic acid (6.5 mL) was stirred at 50° C. for 6 hours. The solvent was concentrated under vacuum. The crude product was purified by reverse phase chromatography and Pre-HPLC with the following conditions. (Column: Xcelect CSH F-phenyl OBD Column, 19*250 mm, 5 μm; Mobile Phase A: Water (10 mmol/L NH₄HCO₃), Mobile Phase B: MeOH—Preparative; Flow rate: 25 mL/min; Gradient: 66% B to 78% B in 8 min, 78% B; Wave Length: 254 nm; RT1(min): 7.72) to afforded 6-(4-((2-aminopyridin-3-yl)methyl)-8-chloro-2-(((2S,4R)-4-fluoro-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine (6.5 mg, 0.01 mmol, 16.6% yield). LCMS (ESI, m/z): 633.3[M+H]+.

Example 49: ¹H NMR (300 MHz, DMSO-d₆. ppm) δ 7.87 (dd, J=4.9, 1.8 Hz, 1H), 7.25 (dd, J=7.3, 1.8 Hz, 1H), 6.95 (s, 1H), 6.72 (s, 2H), 6.50 (dd, J=7.3, 4.9 Hz, 1H), 6.42 (s, 1H), 6.00 (s, 2H), 5.12 (d, J=56.2 Hz, 1H), 4.81 (s, 2H), 4.60 (m, 2H), 4.26 (m, 1H), 4.12 (m, 1H), 3.85 (d, J=5.4 Hz, 2H), 3.47-3.33 (m, 2H), 2.80 (m, 1H), 2.38-2.30 (m, 3H), 2.29 (s, 3H), 2.11-1.94 (m, 1H), 1.90-1.68 (m, 1H)

Example 50: (R)-6-(4-((2-aminopyridin-3-yl)methyl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

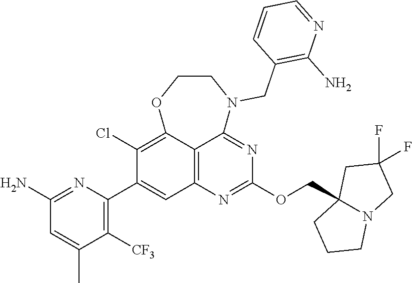 [SMILES: Cc1cc(N)nc(-c2cc3nc(OC[C@]45CCCN4CC(F)(F)C5)nc4c3c(c2Cl)OCCN4Cc2cccnc2N)c1C(F)(F)F]

### Synthetic Route

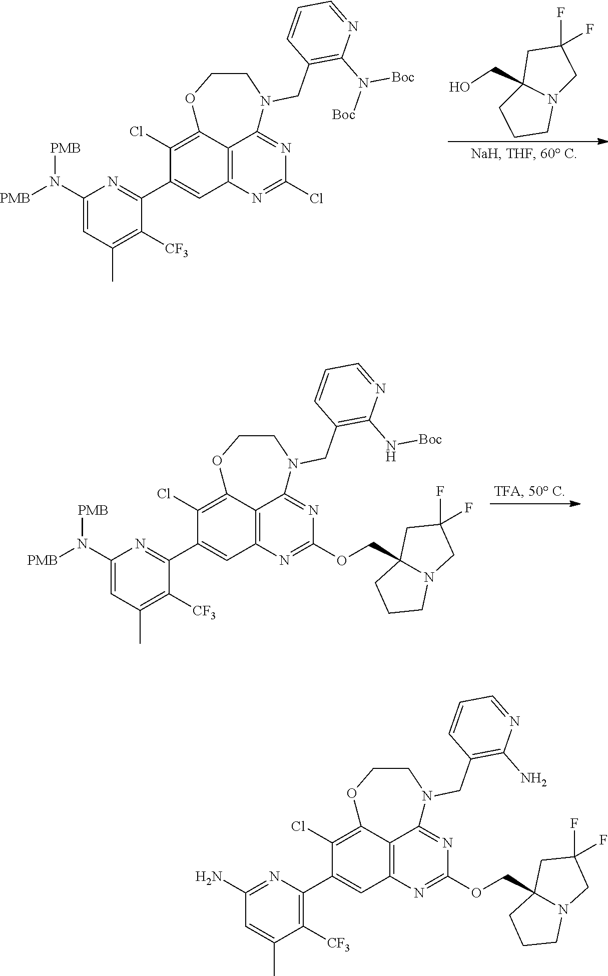

Step 1: tert-butyl (R)-(3-((9-(6-(bis(4-methoxybenzyl)amino)-4-methyl-3-(trifluoromethyl)pyridin-2-yl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-4-yl)methyl)pyridin-2-yl)carbamate

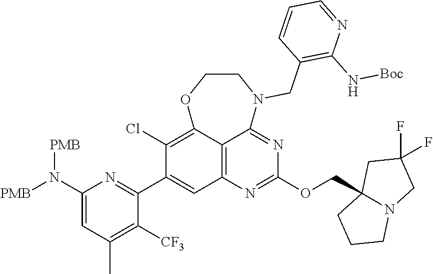

A solution of (R)-(2,2-difluorotetrahydro-1H-pyrrolizin-7a(5H)-yl)methanol (163.2 mg, 0.92 mmol) and sodium hydride (73.7 mg, 1.84 mmol, 60% purity) in tetrahydrofuran (9 mL) was stirred at 0° C. for 15 min. Then tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.05,14]tetradeca-1,3,5(14),6,8-pentaen-13-yl]methyl]-2-pyridyl]-N-tert-butoxycarbonyl-carbamate (90.0 mg, 0.09 mmol) was added and stirred at 60° C. for 1 hour. The solvent was quenched with saturated ammonium chloride solution. The reaction mixture was diluted with dichloromethane. The resulting solution was washed with water, dried over anhydrous sodium sulfate and concentrated under vacuum. The residue was purified by flash chromatography on silica gel eluting with dichloromethane/methanol (20/1) to afford tert-butyl (R)-(3-((9-(6-(bis(4-methoxybenzyl)amino)-4-methyl-3-(trifluoromethyl)pyridin-2-yl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-4-yl)methyl)pyridin-2-yl)carbamate (100.0 mg, 0.09 mmol, 96% yield) as a white solid. LCMS (ESI, m/z): 1017.2 [M+H]⁺.

Step 2: (R)-6-(4-((2-aminopyridin-3-yl)methyl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

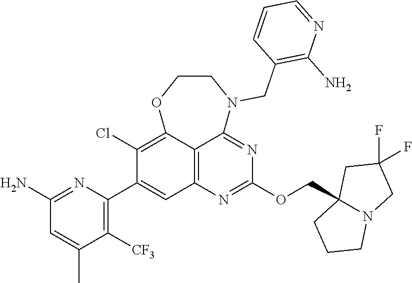 [SMILES: Cc1cc(N)nc(-c2cc3nc(OC[C@]45CCCN4CC(F)(F)C5)nc4c3c(c2Cl)OCCN4Cc2cccnc2N)c1C(F)(F)F]

A solution of tert-butyl (R)-(3-((9-(6-(bis(4-methoxybenzyl)amino)-4-methyl-3-(trifluoromethyl)pyridin-2-yl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-4-yl)methyl)pyridin-2-yl)carbamate (95.0 mg, 0.09 mmol) in 2,2,2-trifluoroacetic acid (3 mL) was stirred at 50° C. for 8 hours. The solvent was concentrated under vacuum to afford crude product. The crude product was purified by reverse phase chromatography and Pre-HPLC with the following conditions. (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 39% B to 69% B in 7 min, 69% B: Wave Length: 254 nm; RT1(min): 6.5; Number Of Runs: 0) to afforded (R)-6-(4-((2-aminopyridin-3-yl)methyl)-8-chloro-2-((2,2-difluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (11.9 mg, 0.018 mmol, 98.9% yield). LCMS (ESI, m/z): 677.3 [M+H]⁺.

Example 50: ¹H NMR (300 MHz, DMSO-d₆, ppm) δ 7.88 (dd, J=4.9, 1.7 Hz, 1H), 7.28 (dd, J=7.4, 1.8 Hz, 1H), 6.94 (s, 1H), 6.72 (s, 2H), 6.51 (dd, J=7.3, 4.9 Hz, 1H), 6.42 (s, 1H), 6.02 (s, 2H), 4.82 (s, 2H), 4.68-4.48 (m, 2H), 4.05-3.89 (m, 2H), 3.85 (d, J=4.7 Hz, 2H), 3.26-3.13 (m, 1H), 3.10-2.90 (m, 2H), 2.75-2.57 (m, 1H), 2.40-2.13 (m, 5H), 1.90 (m, 1H), 1.80 (m, 1H), 1.67 (m, 2H)

Example 51: 1-(2-(9-(6-amino-4-methyl-3-(trifluoromethyl)pyridin-2-yl)-8-chloro-5,6-dihydro-[1,4]oxazepino[5,6,7-de]quinazolin-4-yl)ethyl)pyridin-2(1H)-one

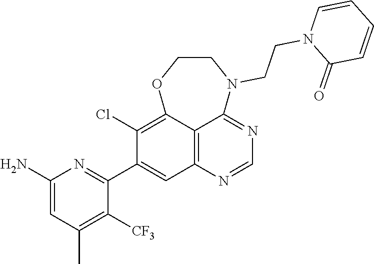 [SMILES: Cc1cc(N)nc(-c2cc3ncnc4c3c(c2Cl)OCCN4CCn2ccccc2=O)c1C(F)(F)F]

### Synthetic Route

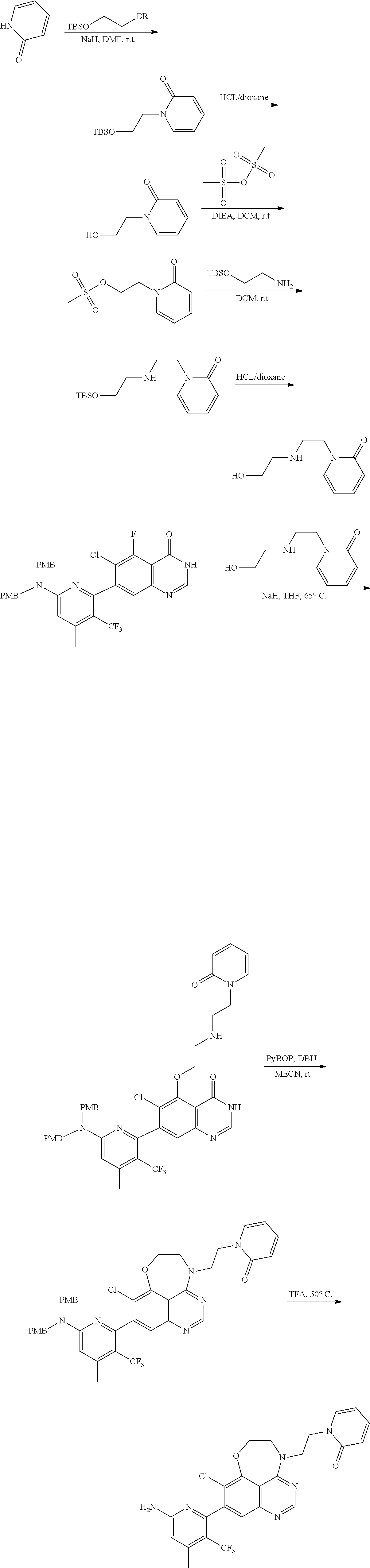

Step 1: 1-(2-(tert-butyldimethylsilyloxy)ethyl)pyridin-2(1H)-one

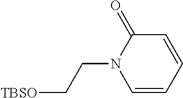 [SMILES: CC(C)(C)[Si](C)(C)OCCn1ccccc1=O]

A solution of 1H-pyridin-2-one (4.00 g, 42.06 mmol), (2-bromoethoxy)(tert-butyl)dimethylsilane (18 mL, 84.12 mmol) and sodium hydride (5.04 g, 126.18 mmol, 60% purity) in N,N-dimethylformamide (40 mL) was stirred at 25° C. for 2 hours. The reaction solution was quenched with saturated ammonium chloride solution and the reaction mixture was diluted with H₂O, extracted with ethyl acetate and washed with saturated brine. Then the organic layer was dried, filtered and evaporated. The residue was purified by flash chromatography on silica gel eluting with dichloromethane/methanol (60/1) to afford 1-[2-[tert-butyl(dimethyl)silyl]oxyethyl]pyridin-2-one (1.36 g, 4.15 mmol, 9.9% yield) as a yellow solid. LC-MS: (ESI, m/z): 254.2 [M+H]

Step 2: 1-(2-hydroxyethyl)pyridin-2(1H)-one. hydrogen chloride

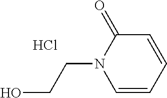

A solution of 1-[2-[tert-butyl(dimethyl)silyl]oxyethyl]pyridin-2-one (1.36 g, 5.37 mmol) in HCl/1,4-dioxane (40.8 mL) was stirred at 25° C. for 2 hours. The solvent was concentrated under vacuum to afford 1-(2-hydroxyethyl)pyridin-2(1H)-one. hydrogen chloride (1.00 g, crude). LC-MS: (ESI, m/z): 140.2 [M+H]

Step 3: 2-(2-oxopyridin-1(2H)-yl)ethyl methanesulfonate

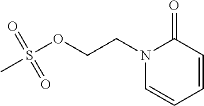

A solution of 1-(2-hydroxyethyl)pyridin-2-one (400.0 mg, crude), methanesulfonicanhydride (651.0 mg, 3.74 mmol) and N,N-diisopropylethylamine (1.6 mL, 9.20 mmol) in dichloromethane (9 mL) was stirred at 25° C. for 1 hour. The solvent was concentrated under vacuum to afford 2-(2-oxo-1-pyridyl)ethyl methanesulfonate (400.0 mg, crude).

Step 4: 1-(2-(2-(tert-butyldimethylsilyloxy)ethylamino)ethyl)pyridin-2(1H)-one

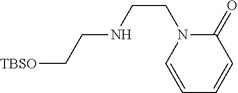 [SMILES: CC(C)(C)[Si](C)(C)OCCNCCn1ccccc1=O]

A solution of 2-[tert-butyl(dimethyl)silyl]oxyethanamine (322.9 mg, 1.84 mmol) and 2-(2-oxo-1-pyridyl)ethyl methanesulfonate (400.0 mg, crude) in dichloromethane (9 mL) was stirred at 25° C. for 1 hour. The solvent was concentrated under vacuum. The residue was purified by flash chromatography on silica gel eluting with dichloromethane/methanol (1/1) to afford 1-[2-[2-[tert-butyl(dimethyl)silyl]oxyethylamino]ethyl]pyridin-2-one (170.0 mg, 0.46 mmol) as a brown solid. LC-MS: (ESI, m/z): 297.2 [M+H]⁺.

Step 5: 1-(2-(2-hydroxyethylamino)ethyl)pyridin-2(1H)-one. hydrogen chloride

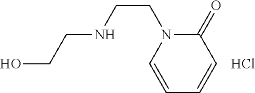 [SMILES: O=c1ccccn1CCNCCO ; Cl]

A solution of 1-[2-[2-[tert-butyl(dimethyl)silyl]oxyethylamino]ethyl]pyridin-2-one (270.0 mg, 0.91 mmol) in dimethyl sulfoxide (5 mL) and HCl in 1,4-dioxane (2.6 mL) was stirred at 25° C. for 0.5 hour. The solvent was concentrated under vacuum to afford 1-[2-(2-hydroxyethylamino)ethyl]pyridin-2-one. hydrogen chloride (165.9 mg, crude) as a brown solid. LC-MS: (ESI, m/z): 183.0[M+H]

Step 6: 7-(6-(bis(4-methoxybenzyl)amino)-4-methyl-3-(trifluoromethyl)pyridin-2-yl)-6-chloro-5-(2-(2-(2-oxopyridin-1(2H)-yl)ethylamino)ethoxy)quinazolin-4(3H)-one

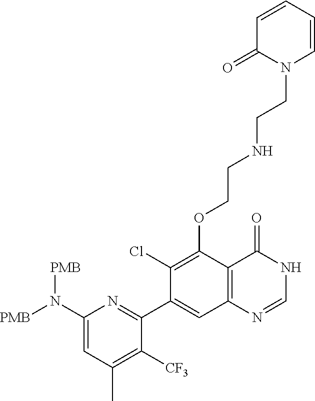

A solution of 1-[2-(2-hydroxyethylamino)ethyl]pyridin-2-one. hydrogen chloride (165.9 mg, crude) and sodium hydride (109.2 mg, 2.73 mmol, 60% purity) in tetrahydrofuran (8 mL) was stirred for 10 min at 0° C. Then 7-[6-[bis[(4-methoxyphenyl)methyl]amino]-4-methyl-3-(trifluoromethyl)-2-pyridyl]-6-chloro-5-fluoro-3H-quinazolin-4-one (279.0 mg, 0.46 mmol) was added and stirred for 2 h at 65° C. for 2 hours. The solvent was quenched with 1N HCl and concentrated under vacuum. The residue was purified by flash chromatography on silica gel eluting with dichloromethane/methanol (20/1) to afford 7-(6-(bis(4-methoxybenzyl)amino)-4-methyl-3-(trifluoromethyl)pyridin-2-yl)-6-chloro-5-(2-(2-(2-oxopyridin-1(2H)-yl)ethylamino)ethoxy)quinazolin-4(3H)-one (110.0 mg, 0.12 mmol, 26.8% yield) as a brown solid. LC-MS: (ESI, m/z): 775.2[M+H]

Step 7: 1-(2-(9-(6-(bis(4-methoxybenzyl)amino)-4-methyl-3-(trifluoromethyl)pyridin-2-yl)-8-chloro-5,6-dihydro-[1,4]oxazepino[5,6,7-de]quinazolin-4-yl)ethyl)pyridin-2(1H)-one

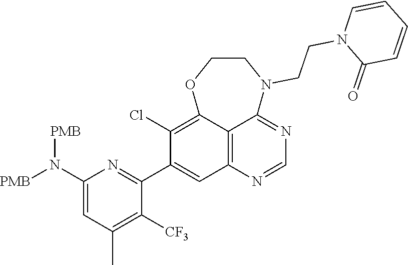

A solution of 7-(6-(bis(4-methoxybenzyl)amino)-4-methyl-3-(trifluoromethyl)pyridin-2-yl)-6-chloro-5-(2-(2-(2-oxopyridin-1(2H)-yl)ethylamino)ethoxy)quinazolin-4(3H)-one (115.0 mg, 0.15 mmol), benzotriazole-1-yl-oxytripyrrolidinophosphonium hexafluorophosphate (100.3 mg, 0.19 mmol) and 1,8-diazabicyclo[5.4.0]undec-7-ene (0.07 mL, 0.45 mmol) in acetonitrile (3.8 mL) was stirred at 25° C. for 0.5 hour. The solvent was concentrated under vacuum. The residue was purified by flash chromatography on silica gel eluting with dichloromethane/methanol (60/1) to afford 1-(2-(9-(6-(bis(4-methoxybenzyl)amino)-4-methyl-3-(trifluoromethyl)pyridin-2-yl)-8-chloro-5,6-dihydro-[1,4]oxazepino[5,6,7-de]quinazolin-4-yl)ethyl)pyridin-2(1H)-one (100.0 mg, 0.12 mmol, 83.7% yield) as a brown solid. LC-MS: (ESI, m/z): 757.2 [M+H]⁺

Step 8: 1-(2-(9-(6-amino-4-methyl-3-(trifluoromethyl)pyridin-2-yl)-8-chloro-5,6-dihydro-[1,4]oxazepino[5,6,7-de]quinazolin-4-yl)ethyl)pyridin-2(1H)-one

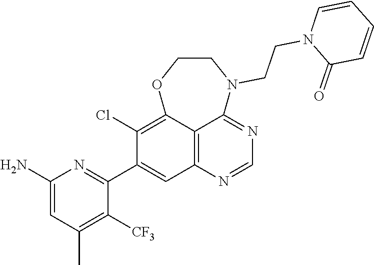 [SMILES: Cc1cc(N)nc(-c2cc3ncnc4c3c(c2Cl)OCCN4CCn2ccccc2=O)c1C(F)(F)F]

A solution of 1-[2-[7-[6-[bis[(4-methoxyphenyl)methyl]amino]-4-methyl-3-(trifluoromethyl)-2-pyridyl]-8-chloro-10-oxa-2,4,13-triazatricyclo[7.4.1.05,14]tetradeca-1,3,5(14),6,8-pentaen-13-yl]ethyl]pyridin-2-one (100.0 mg, 0.13 mmol) in 2,2,2-trifluoroacetic acid (5.6 mL) was stirred at 50° C. for 6 hours. The solvent was concentrated under vacuum to afford crude product. The crude product was purified by reverse phase chromatography and Pre-HPLC with the following conditions (Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 20% B to 42% B in 10 min, 42% B; Wave Length: 254/220 nm; RT1(min): 10.38; Number Of Runs: 0) to afford 1-[2-[7-[6-amino-4-methyl-3-(trifluoromethyl)-2-pyridyl]-8-chloro-10-oxa-2,4,13-triazatricyclo[7.4.1.05,14]tetradeca-1,3,5(14),6,8-pentaen-13-yl]ethyl]pyridin-2-one (23.6 mg, 0.05 mmol, 24.1% yield). LCMS (ESI, m/z): 517.2 [M+H]⁺.

Example 51: ¹H NMR (300 MHz, DMSO-d₆, ppm) δ 8.28 (s, 1H), 7.57 (dd, J=6.8, 2.1 Hz, 1H), 7.34 (m, 1H), 7.12 (s, 1H), 6.73 (s, 2H), 6.43 (s, 1H), 6.30 (d, J=10.0 Hz, 1H), 6.09 (m, 1H), 4.52 (m, 2H), 4.29-4.03 (m, 4H), 3.82 (m, 2H), 2.33 (d, J=2.3 Hz, 3H)

Example 52: 6-[13-[(2-amino-5-chloro-3-pyridyl)methyl]-8-chloro-10-oxa-2,4,13-triazatricyclo[7.4.1.05,14]tetradeca-1,3,5(14),6,8-pentaen-7-yl]-4-methyl-5-(trifluoromethyl)pyridin-2-amine

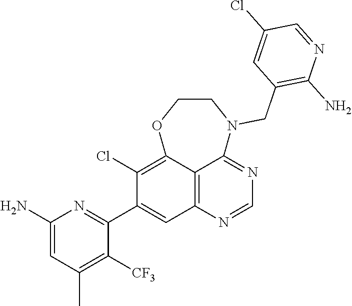 [SMILES: Cc1cc(N)nc(-c2cc3ncnc4c3c(c2Cl)OCCN4Cc2cc(Cl)cnc2N)c1C(F)(F)F]

### Synthetic Route

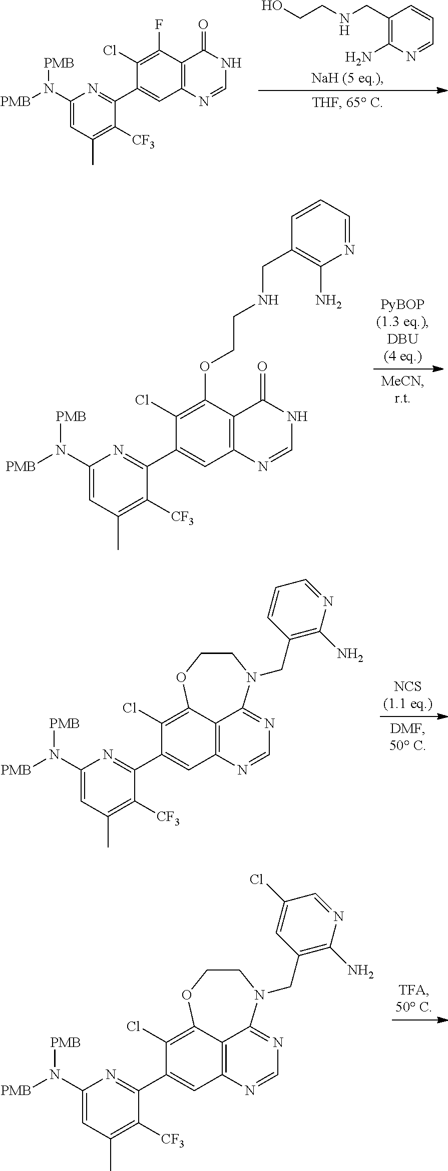

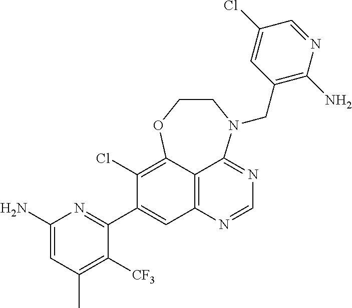 [SMILES: Cc1cc(N)nc(-c2cc3ncnc4c3c(c2Cl)OCCN4Cc2cc(Cl)cnc2N)c1C(F)(F)F]

Step 1: 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloroquinazolin-4(3H)-one

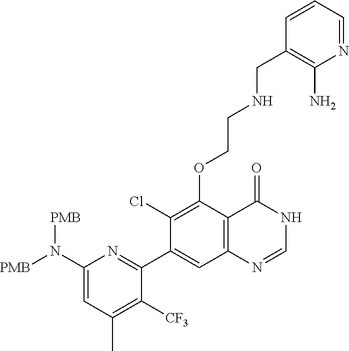

A solution of sodium hydride (117.5 mg, 2.94 mmol, 60% purity) and 2-(((2-aminopyridin-3-yl)methyl)amino)ethan-1-ol (354.8 mg, 1.96 mmol) in tetrahydrofuran (2 mL) was stirred at 0° C. for 5 minutes. Then 7-(6-(bis(4-methoxybenzyl)amino)-4-methyl-3-(trifluoromethyl)pyridin-2-yl)-6-chloro-5-fluoroquinazolin-4(3H)-one (300.0 mg, 0.49 mmol) was added and allowed to heated to 65° C. Then the mixture solution stirred for 2 hours. The reaction was quenched by saturated ammonium chloride solution. The solvent was concentrated under vacuum. The residue was purified by flash chromatography on silica gel eluting with Water/acetonitrile (0-100%) to afford 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (250.0 mg, 0.28 mmol, 58.1% yield) as a yellow solid. LCMS (ESI, m/z): 760.2 [M+H]⁺.

Step 2: 6-[13-[(2-amino-3-pyridyl)methyl]-8-chloro-10-oxa-2,4,13-triazatricyclo[7.4.1.05,14] tetradeca-1,3,5(14),6,8-pentaen-7-yl]-N,N-bis[(4-methoxyphenyl)methyl]-4-methyl-5-(trifluoromethyl)pyridin-2-amine

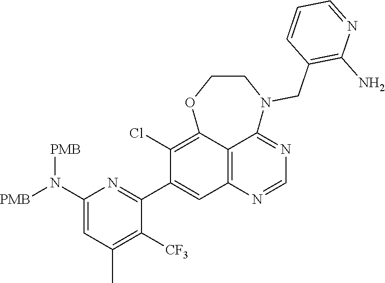

A solution of 5-(2-(((2-aminopyridin-3-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (235.9 mg, 1.55 mmol) in acetonitrile (10 mL) was stirred at 25° C. for 5 min. Then benzotriazol-1-yl-oxytripyrrolidinophosphonium hexafluorophosphate (262.1 mg, 0.50 mmol) was added and stirred at 25° C. for 0.5 hours. After completed, the solvent was concentrated under vacuum. The residue was purified by flash chromatography on silica gel eluting with dichloromethane/methanol (10/1) to afford 6-[13-[(2-amino-3-pyridyl)methyl]-8-chloro-10-oxa-2,4,13-triazatricyclo[7.4.1.05,14]tetradeca-1,3,5(14),6,8-pentaen-7-yl]-N, N-bis[(4-methoxyphenyl)methyl]-4-methyl-5-(trifluoromethyl)pyridin-2-amine (50.0 mg, 0.13 mmol, 32.4% yield) as a yellow solid. LCMS (ESI, m/z): 742.2 [M+H]⁺.

Step 3: 6-[13-[(2-amino-5-chloro-3-pyridyl)methyl]-8-chloro-10-oxa-2,4,13-triazatricyclo[7.4.1.05,14]tetradeca-1,3,5(14),6,8-pentaen-7-yl]-N,N-bis[(4-methoxyphenyl)methyl]-4-methyl-5-(trifluoromethyl)pyridin-2-amine

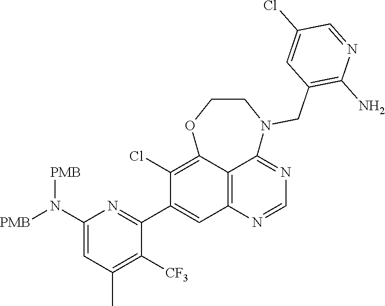

A solution of 6-[13-[(2-amino-3-pyridyl)methyl]-8-chloro-10-oxa-2,4,13-triazatricyclo[7.4.1.05,14]tetradeca-1,3,5(14),6,8-pentaen-7-yl]-N,N-bis[(4-methoxyphenyl)methyl]-4-methyl-5-(trifluoromethyl)pyridin-2-amine (30.0 mg, 0.04 mmol) and N-chlorosuccinimide (5.9 mg, 0.04 mmol) in N,N-dimethylformamide (5 mL) was stirred at 50° C. for 2 hours. After completion, the reaction solution was diluted with water. The resulting solution was extracted with ethyl acetate and the organic layers were combined and washed by water. The organic layer was dried over anhydrous sodium sulfate. The solvent was concentrated under vacuum. The residue was purified by flash chromatography on silica gel eluting with dichloromethane/methanol (10/1) to afford (22.0 mg, 0.039 mmol, 29.8% yield) as a yellow solid. LCMS (ESI, m/z): 776.2 [M+H]⁺.

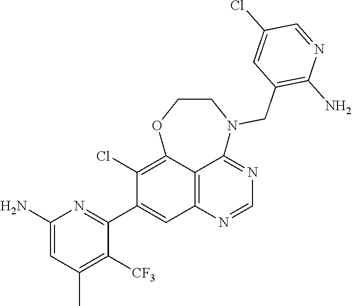 [SMILES: Cc1cc(N)nc(-c2cc3ncnc4c3c(c2Cl)OCCN4Cc2cc(Cl)cnc2N)c1C(F)(F)F]

Step 4: 6-[13-[(2-amino-5-chloro-3-pyridyl)methyl]-8-chloro-10-oxa-2,4,13-triazatricyclo[7.4.1.05,14]tetradeca-1,3,5(14),6,8-pentaen-7-yl]-4-methyl-5-(trifluoromethyl)pyridin-2-amine

A solution of 6-[13-[(2-amino-5-chloro-3-pyridyl)methyl]-8-chloro-10-oxa-2,4,13-triazatricyclo[7.4.1.05,14]tetradeca-1,3,5(14),6,8-pentaen-7-yl]-N,N-bis[(4-methoxyphenyl)methyl]-4-methyl-5-(trifluoromethyl)pyridin-2-amine (22.0 mg, 0.03 mmol) in dichloromethane (3.0 mL) and 2,2,2-trifluoroacetic acid (0.6 mL) was stirred at 50° C. for 3 hours. After completion, the solvent was concentrated under vacuum. The residue was purified by flash chromatography on silica gel eluting with dichloromethane/methanol (5/1) to afford crude 7.5 mg as a yellow solid. The crude product was purified by Prep-HPLC with the following conditions: Column: XBridge Prep OBD C18 Column, 30×150 mm 5 um; Mobile Phase A: Water (10 MMOL/L NH4HCO3), Mobile Phase B: ACN; Flow rate: 60 mL/min; Gradient: 33 B to 63 B in 7 min; 254 nm; RT1: 6.5; to afford 6-[13-[(2-amino-5-chloro-3-pyridyl)methyl]-8-chloro-10-oxa-2,4,13-triazatricyclo[7.4.1.05,14]tetradeca-1,3,5(14),6,8-pentaen-7-yl]-4-methyl-5-(trifluoromethyl)pyridin-2-amine (3.1 mg, 0.0057 mmol, 20.2% yield). LCMS (ESI, m/z): 536.3 [M+H]⁺.

Example 52: ¹H NMR (300 MHz, DMSO-d₆, ppm) δ 8.42 (s, 1H), 7.89 (d, J=2.5 Hz, 1H), 7.35 (d, J=2.5 Hz, 1H), 7.20 (s, 1H), 6.76 (s, 2H), 6.45 (s, 1H), 6.27 (s, 2H), 4.83 (s, 2H), 4.74-4.60 (m, 2H), 3.92 (d, J=4.1 Hz, 2H), 2.36 (s, 3H).

Example 53a & 53b: 6-((R)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine and 6-((S)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

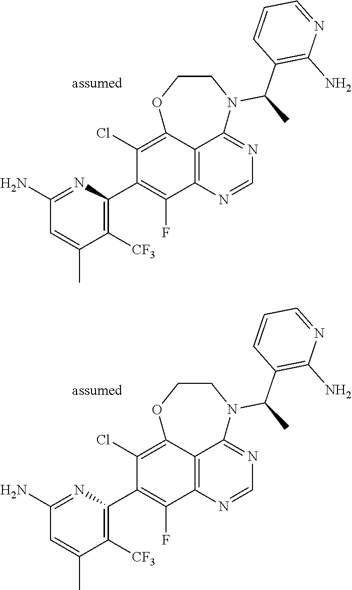

### Synthetic Route

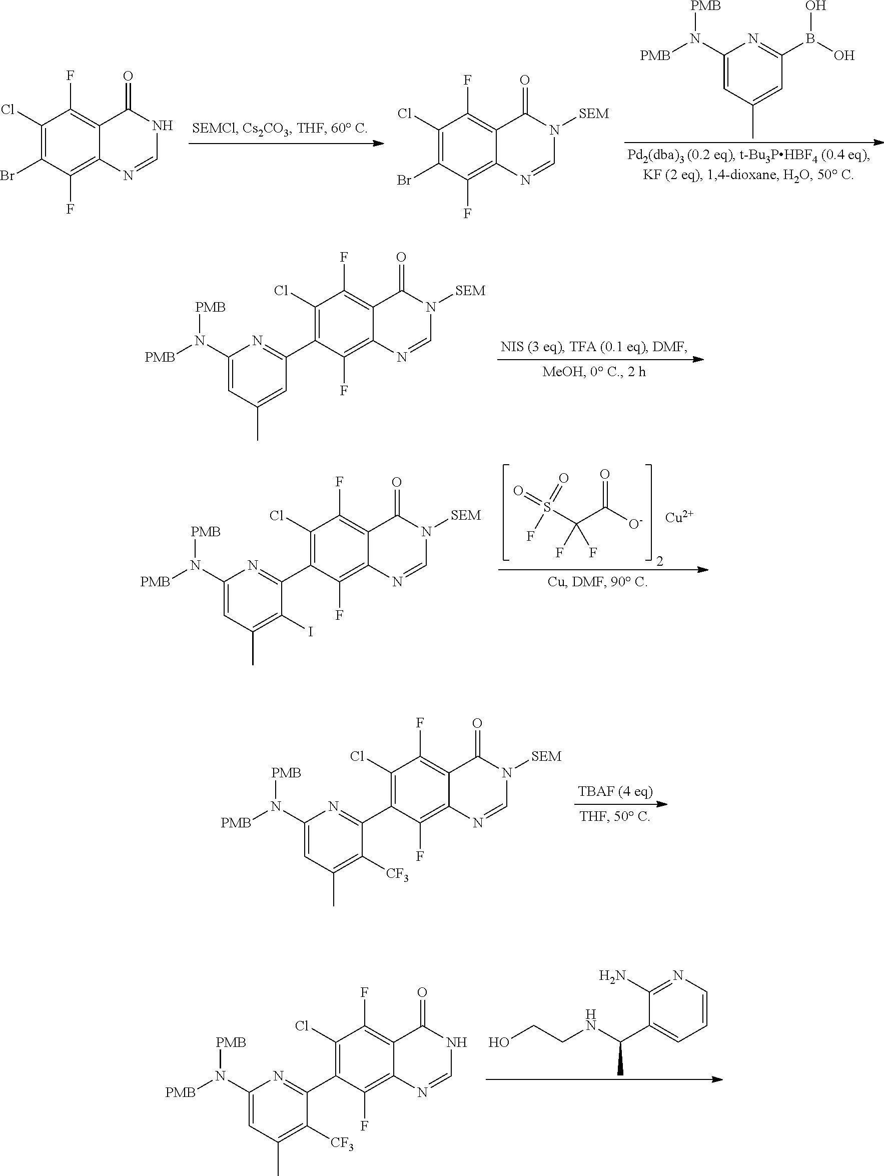

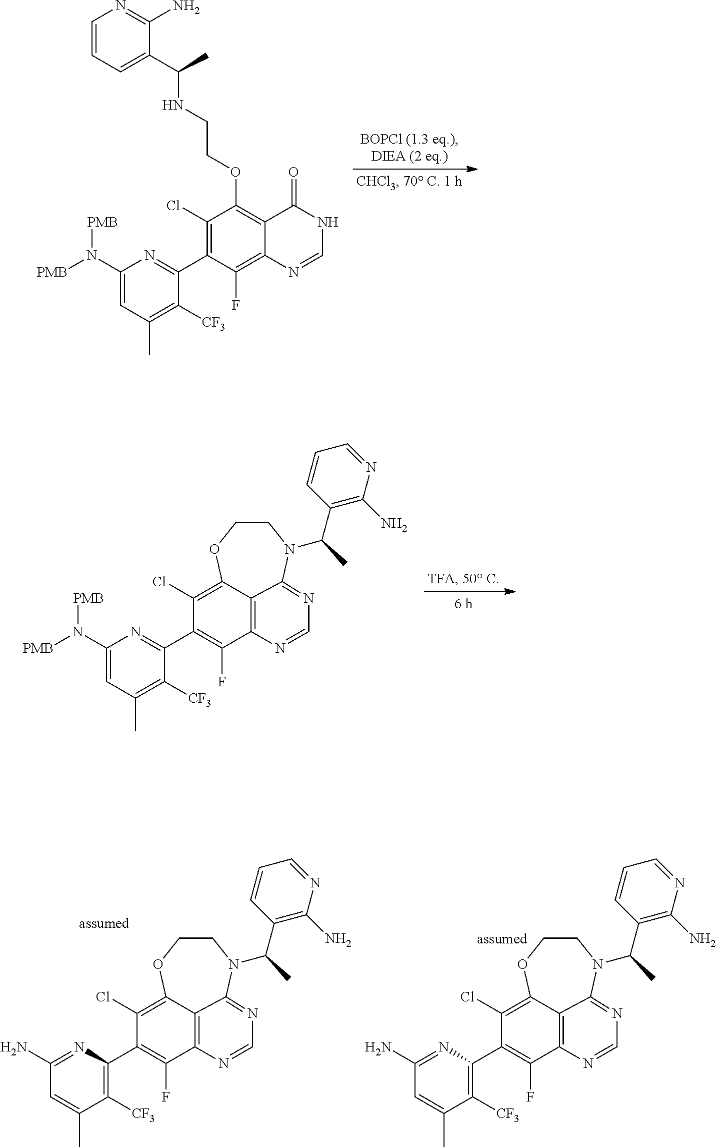

Step 1: 7-bromo-6-chloro-5, 8-difluoro-3-((2-(trimethylsilyl)ethoxy)methyl)quinazolin-4(3H)-one

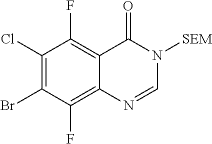 [SMILES: C[Si](C)(C)CCOCn1cnc2c(F)c(Br)c(Cl)c(F)c2c1=O]

The solution of 7-bromo-6-chloro-5,8-difluoroquinazolin-4(3H)-one (5.00 g, 16.92 mmol) and cesium carbonate (16.64 g, 50.77 mmol) in tetrahydrofuran (50 mL) was stirred at 0° C. for 5 minutes. Then 2-(trimethylsilyl)ethoxymethyl chloride (6.0 mL, 33.84 mmol) was added and stirred at 60° C. for 48 hours. After completion, the reaction was quenched with saturated ammonium chloride, extracted with ethyl acetate, dried over anhydrous sodium sulfate and concentrated under vacuum. The residue was purified by flash chromatography on silica gel eluting with petroleum ether/ethyl acetate (10/1) to afford 7-bromo-6-chloro-5,8-difluoro-3-(2-trimethylsilylethoxymethyl)quinazolin-4-one (5.00 g, 11.76 mmol, 69.5% yield) as a white solid. LCMS (ESI, m/z): 424.9 [M+H]⁺

Step 2: 7-(6-(bis(4-methoxybenzyl)amino)-4-methylpyridin-2-yl)-6-chloro-5,8-difluoro-3-((2-(trimethylsilyl)ethoxy)methyl)quinazolin-4(3H)-one

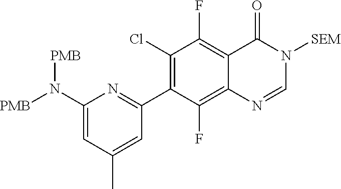

Under nitrogen, a solution of [6-[bis[(4-methoxyphenyl)methyl]amino]-4-methyl-2-pyridyl]boronic acid (3.31 g, 8.46 mmol), 7-bromo-6-chloro-5,8-difluoro-3-((2-(trimethylsilyl)ethoxy)methyl)quinazolin-4(3H)-one (1.80 g, 4.23 mmol), tri-tert-butylphosphine tetrafluoroborate (490.45 mg, 1.69 mmol), potassium fluoride (491.2 mg, 8.46 mmol) and tris(dibenzylideneacetone)dipalladium-chloroform adduct (875.2 mg, 0.85 mmol) in 1,4-dioxane (18.0 mL) and Water (3.6 mL) was stirred at 65° C. for 1 hour. After completion, the reaction was concentrated under vacuum. The residue was diluted with ethyl acetate, washed with water, dried over anhydrous sodium sulfate and concentrated under vacuum. The residue was purified by flash chromatography on silica gel eluting with petroleum ether/ethyl acetate (311) ta afford 7-(6-(bis(4-methoxybenzyl)amino)-4-methylpyridin-2-yl)-6-chloro-5,8-difluoro-3-((2-(trimethylsilyl)ethoxy)methyl)quinazolin-4(3H)-one (1.30 g, 1.87 mmol, 44.4% yield) as a yellow solid. LCMS (ESI, m/z): 693.3 [M+H]⁺

Step 3: 7-(6-(bis(4-methoxybenzyl)amino)-3-iodo-4-methylpyridin-2-yl)-6-chloro-5,8-difluoro-3-((2-(trimethylsilyl)ethoxy)methyl)quinazolin-4(3H)-one

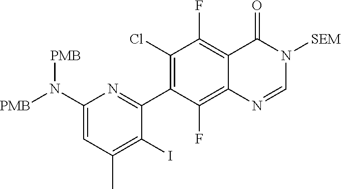

A solution of 7-(6-(bis(4-methoxybenzyl)amino)-4-methylpyridin-2-yl)-6-chloro-5,8-difluoro-3-((2-(trimethylsilyl)ethoxy)methyl)quinazolin-4(3H)-one (1.30 g, 1.88 mmol) and N-iodosuccinimide (1.27 g, 5.63 mmol) in N,N-dimethylformamide (6 mL) and Methyl alcohol (6 mL) was stirred at 0° C. for 2 minutes. Then trifluoroacetic acid (0.02 g, 0.19 mmol) was added and stirred at 0° C. for 2 hours. After completion, the reaction was quenched with saturated sodium sulfite. The resulting solution was extracted with ethyl acetate and the organic layers were combined. The organic layer was dried over anhydrous sodium sulfate and concentrated under vacuum. The residue was purified by flash chromatography on silica gel eluting with petroleum ether/ethyl acetate (3/1) to afford 7-(6-(bis(4-methoxybenzyl)amino)-3-iodo-4-methylpyridin-2-yl)-6-chloro-5,8-difluoro-3-((2-(trimethylsilyl)ethoxy)methyl)quinazolin-4(3H)-one (1.2 g, 1.46 mmol, 78.1% yield) as a yellow solid. LCMS (ESI, m/z): 819.1 [M+H]⁺

Step 4: 7-(6-(bis(4-methoxybenzyl)amino)-4-methyl-3-(trifluoromethyl)pyridin-2-yl)-6-chloro-5,8-difluoro-3-((2-(trimethylsilyl)ethoxy)methyl)quinazolin-4(3H)-one

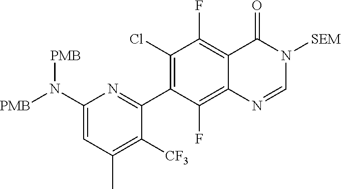

Under nitrogen, a solution of 7-(6-(bis(4-methoxybenzyl)amino)-3-iodo-4-methylpyridin-2-yl)-6-chloro-5,8-difluoro-3-((2-(trimethylsilyl)ethoxy)methyl)quinazolin-4(3H)-one (1.20 g, 1.46 mmol) and copper (0.28 g, 4.39 mmol) in N,N-dimethylformamide (12.0 mL) was stirred 5 minutes at 0° C. Then bis[(2,2-difluoro-2-fluorosulfonyl-acetyl)oxy]copper (1.84 g, 4.39 mmol) was added and stirred at 0° C. for 10 minutes. Then the solution was warmed at room temperature and then under 90° C. and stirred for 1 hour. After completion, the reaction was filtrated; the filtrate was diluted with water, extracted with ethyl acetate, dried over anhydrous sodium sulfate and concentrated under vacuum. The residue was purified by flash chromatography on silica gel eluting with petroleum ether/ethyl acetate (5/1) to afford 7-(6-(bis(4-methoxybenzyl)amino)-4-methyl-3-(trifluoromethyl)pyridin-2-yl)-6-chloro-5,8-difluoro-3-((2-(trimethylsilyl)ethoxy)methyl)quinazolin-4(3H)-one (1.10 g, 1.44 mmol, 98.6% yield) as a white solid. LCMS (ESI, m/z): 761.2 [M+H]⁺

Step 5: 7-(6-(bis(4-methoxybenzyl)amino)-4-methyl-3-(trifluoromethyl)pyridin-2-yl)-6-chloro-5,8-difluoroquinazolin-4(3H)-one

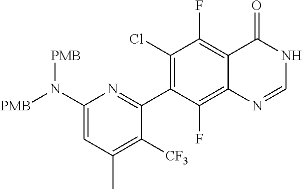

A solution of 7-(6-(bis(4-methoxybenzyl)amino)-4-methyl-3-(trifluoromethyl)pyridin-2-yl)-6-chloro-5,8-difluoro-3-((2-(trimethylsilyl)ethoxy)methyl)quinazolin-4(3H)-one (500.0 mg, 0.66 mmol) and tetrabutylammonium fluoride (685.7 mg, 2.63 mmol) in tetrahydrofuran (5 mL) was stirred at 50° C. for 5 hours. After completion, the reaction mixture was concentrated under reduced pressure. The reaction mixture was diluted with ethyl acetate, washed with water, dried over anhydrous sodium sulfate and concentrated under vacuum. The residue was purified by flash chromatography on silica gel eluting with ethyl acetate/dichloromethane (1/5) to afford 7-(6-(bis(4-methoxybenzyl)amino)-4-methyl-3-(trifluoromethyl)pyridin-2-yl)-6-chloro-5,8-difluoroquinazolin-4(3H)-one (270.0 mg, 0.42 mmol, 65.1% yield) as a yellow solid. LCMS (ESI, m/z): 631.2 [M+H]⁺.

Step 6: 5-(2-(((R)-1-(2-aminopyridin-3-yl)ethyl)amino)ethoxy)-7-(6-(bis(4-methoxybenzyl)amino)-4-methyl-3-(trifluoromethyl)pyridin-2-yl)-6-chloro-8-fluoroquinazolin-4(3H)-one

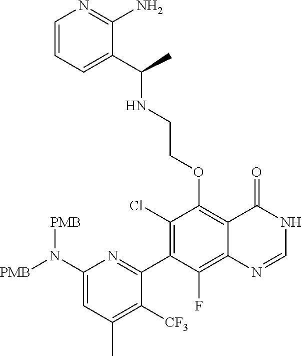

A solution of sodium hydride (85.5 mg, 2.14 mmol, 60% purity) was added to (R)-2-((1-(2-aminopyridin-3-yl)ethyl)amino)ethan-1-ol (155.1 mg, 0.86 mmol) in tetrahydrofuran (1.0 mL) cooled to 0° C. Then 7-(6-(bis(4-methoxybenzyl)amino)-4-methyl-3-(trifluoromethyl)pyridin-2-yl)-6-chloro-5,8-difluoroquinazolin-4(3H)-one (270.0 mg, 0.43 mmol) was added and stirred at 0° C. for 5 minutes, Then the reaction was stirred at 65° C. for 20 minutes. After completion, the reaction was quenched with hydrochloric acid. The solvent was concentrated under vacuum. The residue was purified by flash chromatography on silica gel eluting with acetonitrile/water (4/1) to afford 5-(2-(((R)-1-(2-aminopyridin-3-yl)ethyl)amino)ethoxy)-7-(6-(bis(4-methoxybenzyl)amino)-4-methyl-3-(trifluoromethyl)pyridin-2-yl)-6-chloro-8-fluoroquinazolin-4(3H)-one (200.0 mg, 0.25 mmol, 59% yield) as a yellow solid. LCMS (ESI, m/z): 792.3 [M+H]⁺.

Step 7: 6-(4-((R)-1-(2-aminopyridin-3-yl)ethyl)-8-chloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

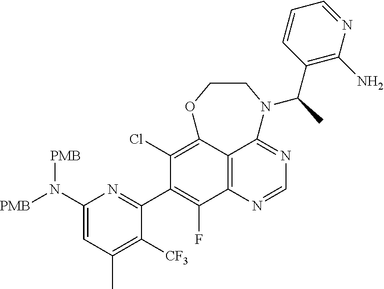

A solution of 5-(2-(((R)-1-(2-aminopyridin-3-yl)ethyl)amino)ethoxy)-7-(6-(bis(4-methoxybenzyl)amino)-4-methyl-3-(trifluoromethyl)pyridin-2-yl)-6-chloro-8-fluoroquinazolin-4(3H)-one (200.0 mg, 0.25 mmol), N,N-diisopropylethylamine (162.8 mg, 1.26 mmol) and in chloroform (3.0 mL) was stirred at 25° C. for 3 minutes. Then bis(2-oxo-3-oxazolidinyl)phosphinic chloride (128.2 mg, 0.50 mmol) was added and stirred at 70° C. for 1 hour. After completion, the reaction mixture was diluted with dichloromethane. The resulting solution was washed with water, dried over anhydrous sodium sulfate and concentrated under vacuum. The residue was purified by flash chromatography on silica gel eluting with dichloromethane/methanol (94/6) to afford 6-(4-((R)-1-(2-aminopyridin-3-yl)ethyl)-8-chloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (110.0 mg, 0.14 mmol, 56.3% yield) as a white solid. LCMS (ESI, m/z): 774.2 [M+H]⁺.

Step 8: 6-((R)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine and 6-((S)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

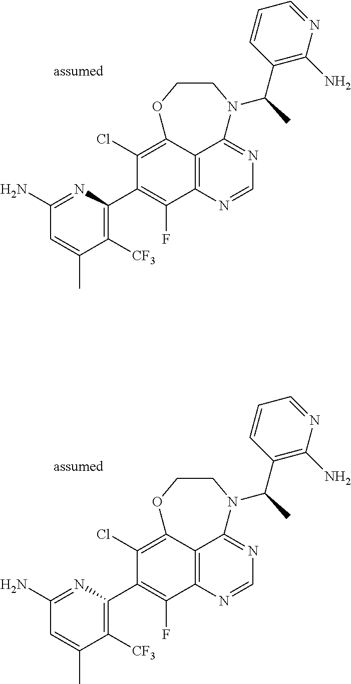

A solution of 6-(4-((R)-1-(2-aminopyridin-3-yl)ethyl)-8-chloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (100.0 mg, 0.13 mmol) in 2,2,2-trifluoroacetic acid (2.0 mL) was stirred at 50° C. for 8 hours. After completion, the solvent was concentrated under vacuum. The residue was purified by flash chromatography on silica gel eluting with acetonitrile/water (4/1) to afford 50 mg crude. The crude product was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 29% B to 59% B in 7 min; Wave Length: 254 nm; RT1(min): 6.5 to afford 6-((R)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (14.6 mg, 0.027 mmol, 21.2% yield) and 6-((S)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (15.2 mg, 0.028 mmol, 22% yield). The stereo chemistry of title compounds was arbitrarily assigned.

Example 53a: ¹H NMR (400 MHz, DMSO-d6, ppm) δ 8.57 (s, 1H), 7.97 (dd, J=4.9, 1.7 Hz, 1H), 7.70-7.56 (m, 1H), 6.83 (s, 2H), 6.72-6.61 (m, 1H), 6.49 (s, 1H), 6.37 (q, J=6.8 Hz, 1H), 5.69 (s, 2H), 4.49-4.38 (m, 1H), 4.29-4.15 (m, 1H), 3.64 (dd, J=15.6, 7.2 Hz, 1H), 3.39 (dd, J=15.4, 6.2 Hz, 1H), 2.36 (s, 3H), 1.56 (d, J=6.8 Hz, 3H). LCMS (ESI, m/z): 534.1 [M+H]⁺.

Example 53b: ¹H NMR (400 MHz, DMSO-d₆) δ 8.57 (s, 1H), 7.96 (dd, J=4.9, 1.7 Hz, 1H), 7.64 (dd, J=7.5, 1.9 Hz, 1H), 6.83 (s, 2H), 6.66 (dd, J=7.5, 4.9 Hz, 1H), 6.51-6.45 (m, 1H), 6.42-6.34 (m, 1H), 5.76 (s, 2H), 4.59-4.46 (m, 1H), 4.35-4.24 (m, 1H), 3.69 (dd, J=15.5, 6.6 Hz, 1H), 3.40-3.33 (m, 1H), 2.36 (s, 3H), 1.58 (d, J=6.8 Hz, 3H). LCMS (ESI, m/z): 534.1 [M+H]⁺.

Example 54: 6-(4-((2-aminopyridin-3-yl)methyl)-8-chloro-2-((3-(dimethylamino)oxetan-3-yl)methoxy)-5,6-dihydro-4H-[1,4]oxazepino[5,6,7-de]quinazolin-9-yl)-4-methyl-5-(trifluoromethyl)pyridin-2-amine

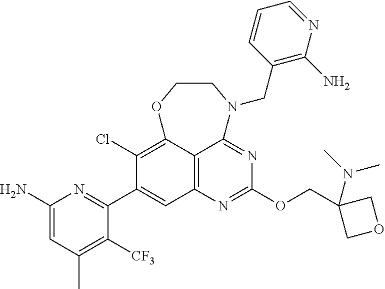 [SMILES: Cc1cc(N)nc(-c2cc3nc(OCC4(N(C)C)COC4)nc4c3c(c2Cl)OCCN4Cc2cccnc2N)c1C(F)(F)F]

### Synthetic Route

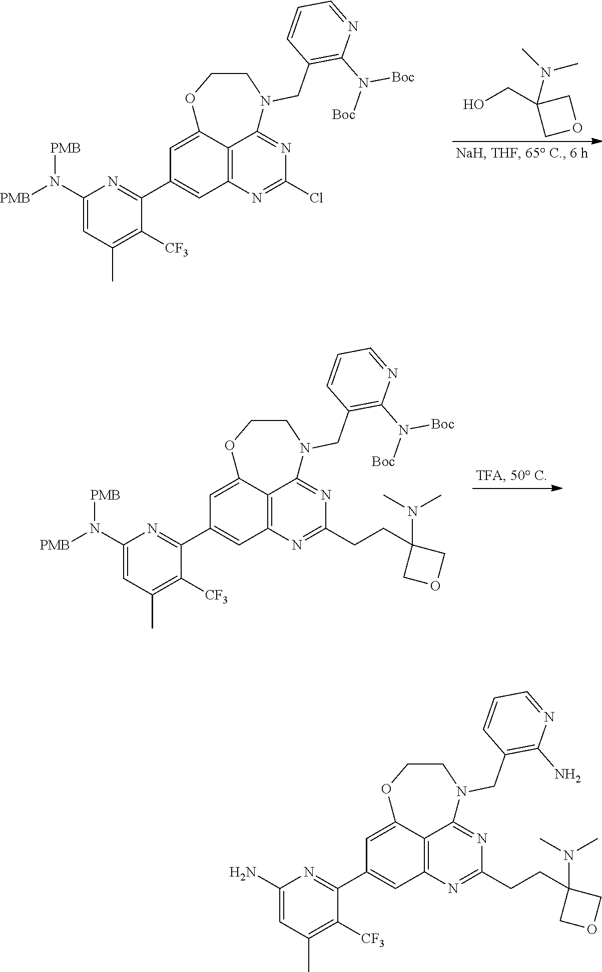

Step 1: tert-butyl N-[3-[[7-[6-[bis[(4-methoxyphenyl)methyl]amino]-4-methyl-3-(trifluoromethyl)-2-pyridyl]-8-chloro-3-[[3-(dimethylamino)oxetan-3-yl]methoxy]-10-oxa-2,4,13-triazatricyclo[7.4.1.05,14]tetradeca-1,3,5(14),6,8-pentaen-13-yl]methyl]-2-pyridyl]-N-tert-butoxycarbonyl-carbamate

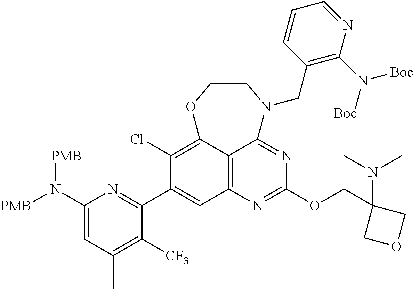

A solution of [3-(dimethylamino)oxetan-3-yl]methanol (40.2 mg, 0.30 mmol) and sodium hydride (20.4 mg, 0.51 mmol, 60% purity) in tetrahydrofuran (1.5 mL) was stirred at 0° C. for 10 minutes. Then tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.05,14]tetradeca-1,3,5(14),6,8-pentaen-13-yl]methyl]-2-pyridyl]-N-tert-butoxycarbonyl-carbamate (100.0 mg, 0.10 mmol) was added and stirred at 65° C. for 2 hours. After completion, the reaction was quenched with hydrochloric acid. The reaction mixture was concentrated under reduced pressure. The residue was purified by flash chromatography on silica gel eluting with methanol/dichloromethane (1/20) to afford tert-butyl N-[3-[[7-[6-[bis[(4-methoxyphenyl)methyl]amino]-4-methyl-3-(trifluoromethyl)-2-pyridyl]-8-chloro-3-[[3-(dimethylamino)oxetan-3-yl]methoxy]-10-oxa-2,4,13-triazatricyclo[7.4.1.05,14]tetradeca-1,3,5(14),6,8-pentaen-13-yl]methyl]-2-pyridyl]-N-tert-butoxycarbonyl-carbamate (60.0 mg, 0.056 mmol, 54.9% yield) as a light yellow solid. LC-MS: (ESI, m/z): 1071.3 [M+H]⁺

Step 2: 6-(4-((2-aminopyridin-3-yl)methyl)-8-chloro-2-((3-(dimethylamino)oxetan-3-yl)methoxy)-5,6-dihydro-4H-[1,4]oxazepino[5,6,7-de]quinazolin-9-yl)-4-methyl-5-(trifluoromethyl)pyridin-2-amine

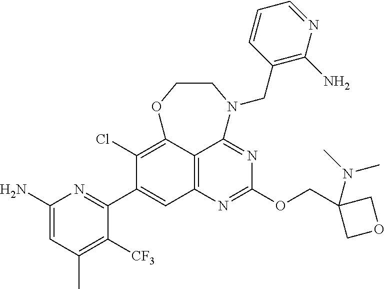 [SMILES: Cc1cc(N)nc(-c2cc3nc(OCC4(N(C)C)COC4)nc4c3c(c2Cl)OCCN4Cc2cccnc2N)c1C(F)(F)F]

A solution of tert-butyl N-[3-[[7-[6-[bis[(4-methoxyphenyl)methyl]amino]-4-methyl-3-(trifluoromethyl)-2-pyridyl]-8-chloro-3-[[3-(dimethylamino)oxetan-3-yl]methoxy]-10-oxa-2,4,13-triazatricyclo[7.4.1.05,14]tetradeca-1,3,5(14),6,8-pentaen-13-yl]methyl]-2-pyridyl]-N-tert-butoxycarbonyl-carbamate (60.0 mg, 0.056 mmol) in 2,2,2-trifluoroacetic acid (1 mL) was stirred at 50° C. for 6 hours. After completion, the reaction mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 27% B to 57% B in 7 min; Wave Length: 254 nm) to afford 6-(4-((2-aminopyridin-3-yl)methyl)-8-chloro-2-((3-(dimethylamino)oxetan-3-yl)methoxy)-5,6-dihydro-4H-[1,4]oxazepino[5,6,7-de]quinazolin-9-yl)-4-methyl-5-(trifluoromethyl)pyridin-2-amine (19.1 mg, 0.030 mmol, 53.5% yield). LC-MS: (ESI, m/z): 631.2 [M+H]⁺

Example 54: ¹H NMR (400 MHz, DMSO-d₆) δ 7.95-7.79 (m, 1H), 7.39-7.22 (m, 1H), 6.99 (s, 1H), 6.74 (s, 2H), 6.56-6.48 (m, 1H), 6.44 (s, 1H), 6.02 (s, 2H), 4.87-4.78 (m, 2H), 4.71-4.60 (m, 2H), 4.60-4.55 (m, 1H), 4.53-4.46 (m, 1H), 4.38 (d, J=6.1 Hz, 2H), 4.33 (d, J=6.1 Hz, 2H), 3.99-3.84 (m, 2H), 2.41-2.31 (m, 3H), 2.14 (s, 6H).

Example 55: 3-(((9-(6-amino-4-methyl-3-(trifluoromethyl)pyridin-2-yl)-4-((2-aminopyridin-3-yl)methyl)-8-chloro-5,6-dihydro-4H-[1,4]oxazepino[5,6,7-de]quinazolin-2-yl)oxy)methyl)-3-methylthietane 1,1-dioxide

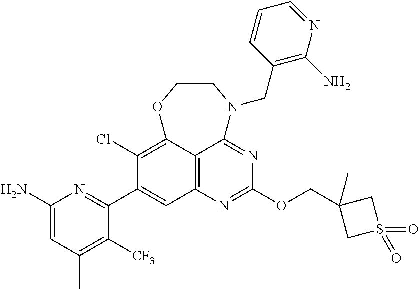 [SMILES: Cc1cc(N)nc(-c2cc3nc(OCC4(C)CS(=O)(=O)C4)nc4c3c(c2Cl)OCCN4Cc2cccnc2N)c1C(F)(F)F]

### Synthetic Route

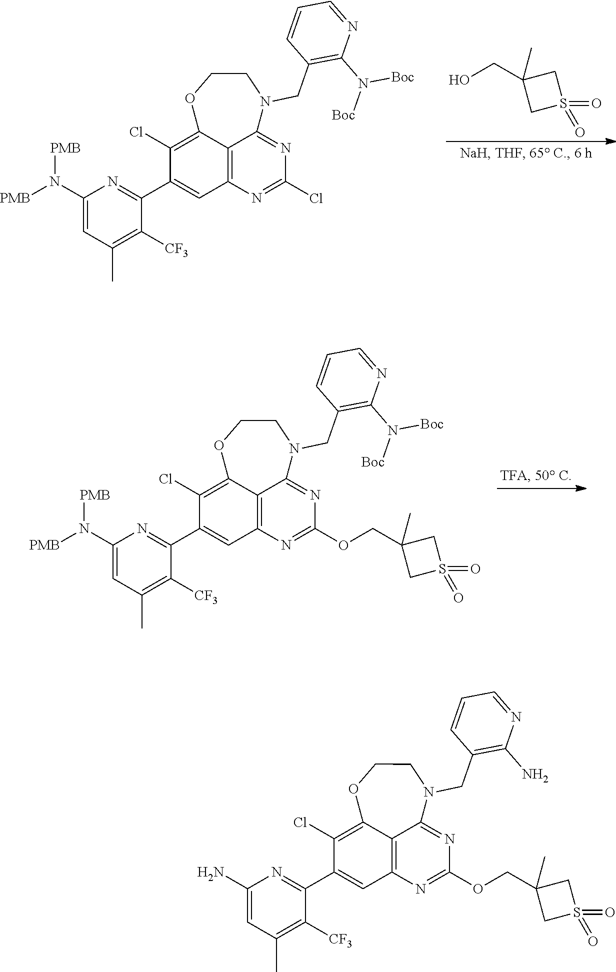

Step 1: 3-(((4-((2-aminopyridin-3-yl)methyl)-9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-2-yl)oxy)methyl)-3-methylthietane 1,1-dioxide

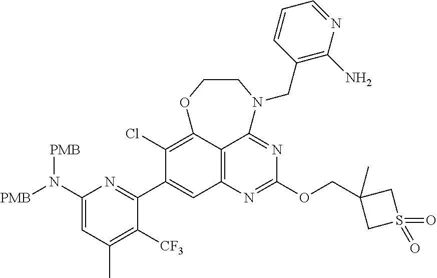

A solution of sodium hydride (14.7 mg, 0.61 mmol) and (3-methyl-1,1-dioxo-thietan-3-yl)methanol (46.1 mg, 0.31 mmol) in tetrahydrofuran (3 mL) was stirred at 0° C. for 20 minutes. Then tert-butyl N-[3-[[7-[6-[bis[(4-methoxyphenyl)methyl]amino]-4-methyl-3-(trifluoromethyl)-2-pyridyl]-3,8-dichloro-10-oxa-2,4,13-triazatricyclo[7.4.1.05,14]tetradeca-1,3,5(14),6,8-pentaen-13-yl]methyl]-2-pyridyl]-N-tert-butoxycarbonyl-carbamate (150.0 mg, 0.15 mmol) was added and stirred at 65° C. for 1 hour. After completion, the reaction mixture was quenched with saturated ammonium chloride, diluted with water and extracted with ethyl acetate. The combined organic layers were dried over anhydrous sodium sulfate and concentrated under vacuum to afford the crude product. The residue was purified by flash chromatography on silica gel eluting with dichloromethane/methanol (10/1) to afford 3-(((4-((2-aminopyridin-3-yl)methyl)-9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-2-yl)oxy)methyl)-3-methylthietane 1,1-dioxide (70.0 mg, 0.07 mmol, 51.2% yield) as a yellow solid. LC-MS: (ESI, m/z): 890.3 [M+H]⁺

Step 2: 3-(((9-(6-amino-4-methyl-3-(trifluoromethyl)pyridin-2-yl)-4-((2-aminopyridin-3-yl)methyl)-8-chloro-5,6-dihydro-4H-[1,4]oxazepino[5,6,7-de]quinazolin-2-yl)oxy)methyl)-3-methylthietane 1,1-dioxide

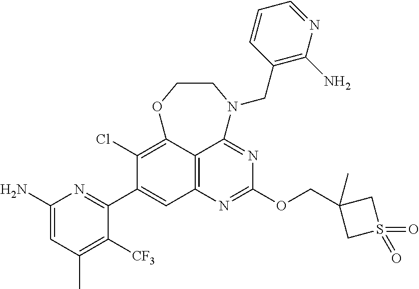 [SMILES: Cc1cc(N)nc(-c2cc3nc(OCC4(C)CS(=O)(=O)C4)nc4c3c(c2Cl)OCCN4Cc2cccnc2N)c1C(F)(F)F]

A solution of 3-(((4-((2-aminopyridin-3-yl)methyl)-9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-2-yl)oxy)methyl)-3-methylthietane 1,1-dioxide (70.0 mg, 0.07 mmol) and trifluoroacetic acid (2 mL) was stirred at 50° C. for 8 hours. After completion, the reaction mixture was concentrated under reduced pressure. The residue was purified by flash chromatography on silica gel eluting with acetonitrile/water (1/1) to afford 3-(((9-(6-amino-4-methyl-3-(trifluoromethyl)pyridin-2-yl)-4-((2-aminopyridin-3-yl)methyl)-8-chloro-5,6-dihydro-4H-[1,4]oxazepino[5,6,7-de]quinazolin-2-yl)oxy)methyl)-3-methylthietane 1,1-dioxide (21.7 mg, 0.03 mmol, 42.5% yield). LC-MS: (ESI, m/z): 650.2 [M+H]⁺

Example 55: ¹H NMR (300 MHz, DMSO-d6, ppm) δ 7.95-7.86 (m, 1H), 7.37-7.28 (m, 1H), 6.99 (d, J=0.7 Hz, 1H), 6.75 (s, 2H), 6.59-6.49 (m, 1H), 6.45 (s, 1H), 6.07 (s, 2H), 4.84 (s, 2H), 4.68-4.55 (m, 2H), 4.42-4.27 (m, 2H), 4.16 (d, J=14.3 Hz, 2H), 3.91 (d, J=14.3 Hz, 4H), 2.36 (d, J=2.3 Hz, 3H), 1.41 (s, 3H).

Example 56: 4-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridazin-3-amine

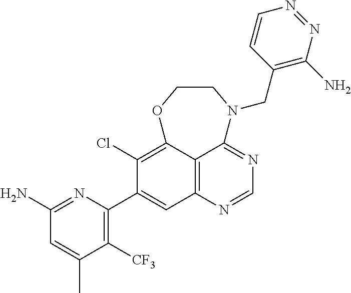 [SMILES: Cc1cc(N)nc(-c2cc3ncnc4c3c(c2Cl)OCCN4Cc2ccnnc2N)c1C(F)(F)F]

### Synthetic Route

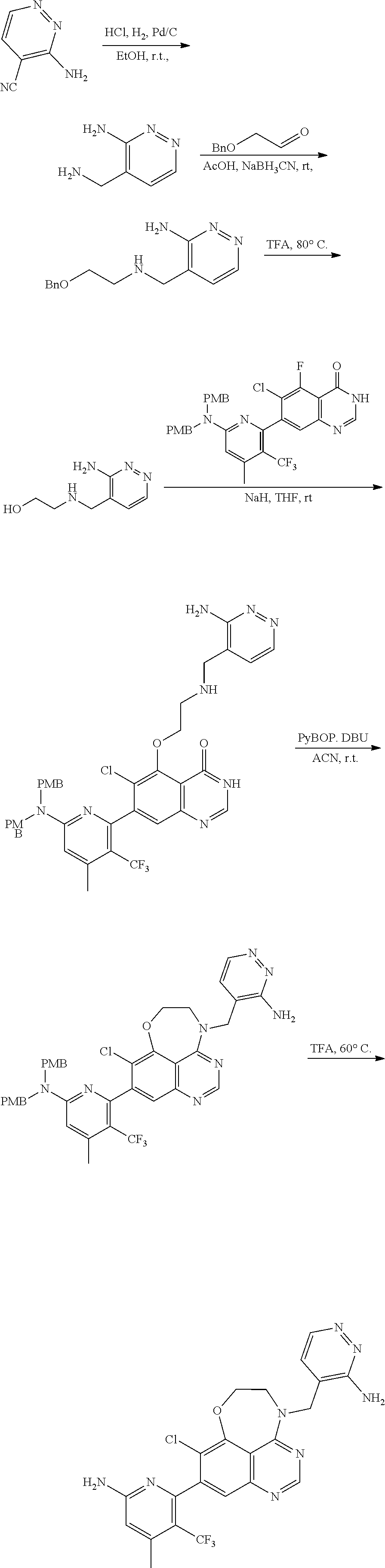

Step 1: 4-(aminomethyl)pyridazin-3-amine

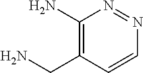

To a mixture of 3-aminopyridazine-4-carbonitrile (2.00 g, 16.65 mmol) in hydrochloric acid (0.1 mL, 0.2 mmol, 2 N) in ethanol (20 mL) was added Pd/C (2.00 g, 6.33 mmol), and the mixture was stirred for 4 hours at 25° C. under hydrogen. After filtration, the organic layer was concentrated under vacuum. The crude would be directly used in the next step without purification. This resulted in 4-(aminomethyl)pyridazin-3-amine (1.70 g, crude) as a brown solid. LCMS (ESI, m/z): 124.1 [M+H]⁺.

Step 2: 4-(((2-(benzyloxy)ethyl)amino)methyl)pyridazin-3-amine

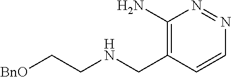 [SMILES: Nc1nnccc1CNCCOCc1ccccc1]

A solution of 4-(aminomethyl)pyridazin-3-amine (800.0 mg, crude), benzyloxyacetaldehyde (0.91 mL, 6.46 mmol), sodium cyanoborohydride (410.0 mg, 6.51 mmol) and acetic acid (0.07 mL, 1.17 mmol) in methyl alcohol (8 mL) was stirred at 25° C. for 2 hours. After completion, the resulting solution was diluted with water, extracted with ethyl acetate. The organic layers was washed with brine, dried over anhydrous sodium sulfate and concentrated. The residue was purified by flash chromatography on silica gel eluting with petroleum ether/ethyl acetate (1/1). This resulted in 4-(((2-(benzyloxy)ethyl)amino)methyl)pyridazin-3-amine (300.0 mg, 1.16 mmol, 18% yield) as a red oil. LCMS (ESI, m/z): 259.1 [M+H]⁺.

Step 3: 2-(((3-aminopyridazin-4-yl)methyl)amino)ethan-1-ol

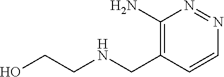 [SMILES: Nc1nnccc1CNCCO]

To a mixture of 4-(((2-(benzyloxy)ethyl)amino)methyl)pyridazin-3-amine (200.0 mg, 0.77 mmol) in 2,2,2-trifluoroacetic acid (2 mL), the mixture was stirred for overnight at 80° C. After completion, the organic layer was concentrated under vacuum. The crude would be directly used in the next step without purification. This resulted in 2-(((3-aminopyridazin-4-yl)methyl)amino)ethan-1-ol (110.0 mg, crude) as a red oil. LCMS (ESI, m/z): 169.1 [M+H]⁺.

Step 4: 5-(2-(((3-aminopyridazin-4-yl)methyl)amino)ethoxy)-7-(6-(bis(4-methoxybenzyl)amino)-4-methyl-3-(trifluoromethyl)pyridin-2-yl)-6-chloroquinazolin-4(3H)-one

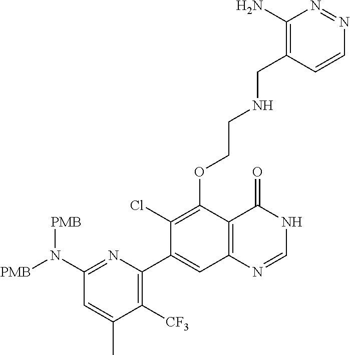

To a mixture of sodium hydride (18 mg, 0.75 mmol) and 2-(((3-aminopyridazin-4-yl)methyl)amino)ethan-1-ol (45 mg, 0.27 mmol) in tetrahydrofuran (2 mL) was stirred for min at 0° C. Then 7-(6-(bis(4-methoxybenzyl)amino)-4-methyl-3-(trifluoromethyl)pyridin-2-yl)-6-chloro-5-fluoroquinazolin-4(3H)-one (150.0 mg, 0.24 mmol) was added at 0° C., the mixture was stirred for 1 hour at 65° C. After completion, the reaction was quenched by saturated ammonium chloride solution. The resulting solution was diluted with water and extracted with ethyl acetate. The organic layer was washed with brine, dried over anhydrous sodium sulfate and concentrated. The residue was purified by flash chromatography on silica gel eluting with petroleum ether/ethyl acetate (1/1). This resulted in 5-(2-(((3-aminopyridazin-4-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (100.0 mg, 0.13 mmol, 53.7% yield) as a yellow solid. LCMS (ESI, m/z): 761.3 [M+H]⁺.

Step 5: 4-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridazin-3-amine

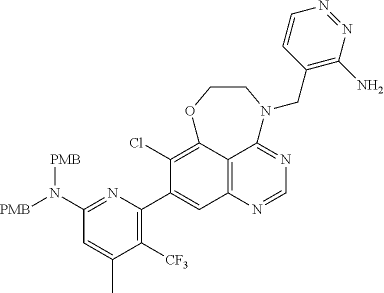

A mixture of 5-(2-(((3-aminopyridazin-4-yl)methyl)amino)ethoxy)-7-(6-(bis(4-methoxybenzyl)amino)-4-methyl-3-(trifluoromethyl)pyridin-2-yl)-6-chloroquinazolin-4(3H)-one (100.0 mg, 0.13 mmol), benzotriazole-1-yl-oxytripyrrolidinophosphonium hexafluorophosphate (102.0 mg, 0.2 mmol) and 1,8-diazabicyclo[5.4.0]undec-7-ene (0.06 mL, 0.39 mmol) in acetonitrile (1 mL) was stirred at 25° C. for 2 hours. After completion, the resulting solution was diluted with water and extracted with ethyl acetate. The organic layer was washed with brine, dried over anhydrous sodium sulfate and concentrated. The residue was purified by flash chromatography on silica gel eluting with petroleum ether/ethyl acetate (3/2). This resulted in 4-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridazin-3-amine (70.0 mg, 0.09 mmol, 71.7% yield) as a light yellow solid. LCMS (ESI, m/z): 743.2 [M+H]⁺.

Step 6: 4-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridazin-3-amine

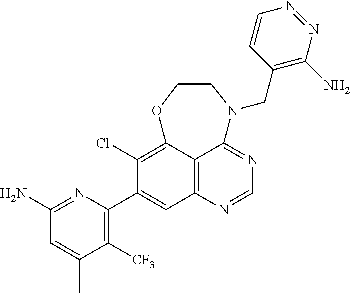 [SMILES: Cc1cc(N)nc(-c2cc3ncnc4c3c(c2Cl)OCCN4Cc2ccnnc2N)c1C(F)(F)F]

To a mixture of 4-((9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)methyl)pyridazin-3-amine (70.0 mg, 0.09 mmol) in 2,2,2-trifluoroacetic acid (1 mL), the mixture was stirred for 4 hours at 60° C. After completion, the solution was concentrated under vacuum. The crude product was purified by Prep-HPLC with the condition: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 29% B to 59% B in 7 min; Wave Length: 254 nm; RT1(min): 6.5. This resulted in 4-((9-(6-amino-4-methyl-3-(trifluoromethyl)pyridin-2-yl)-8-chloro-5,6-dihydro-4H-[1,4]oxazepino[5,6,7-de]quinazolin-4-yl)methyl)pyridazin-3-amine (12.2 mg, 0.02 mmol, 25.8% yield). LCMS (ESI, m/z): 503.00 [M+H]⁺.

Example 56: ¹H NMR (300 MHz, DMSO-de) 68.37 (s, 1H), 8.35 (d, J=4.7 Hz, 1H), 7.21 (s, 1H), 7.02 (d, J=4.8 Hz, 1H), 6.76 (s, 2H), 6.45 (s, 1H), 6.38 (s, 2H), 4.80 (s, 2H), 4.76-4.69 (dd, J=7.6, 4.0 Hz, 2H), 3.98 (d, J=5.4 Hz, 2H), 2.36 (d, J=1.5 Hz, 3H).

Example 57: 6-(4-((2-amino-4-(difluoromethyl)pyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

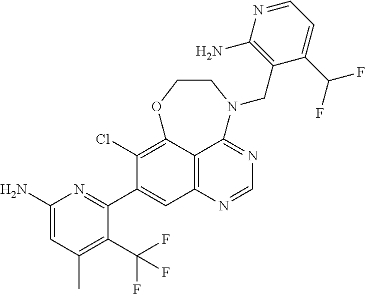 [SMILES: Cc1cc(N)nc(-c2cc3ncnc4c3c(c2Cl)OCCN4Cc2c(C(F)F)ccnc2N)c1C(F)(F)F]

### Synthetic Route

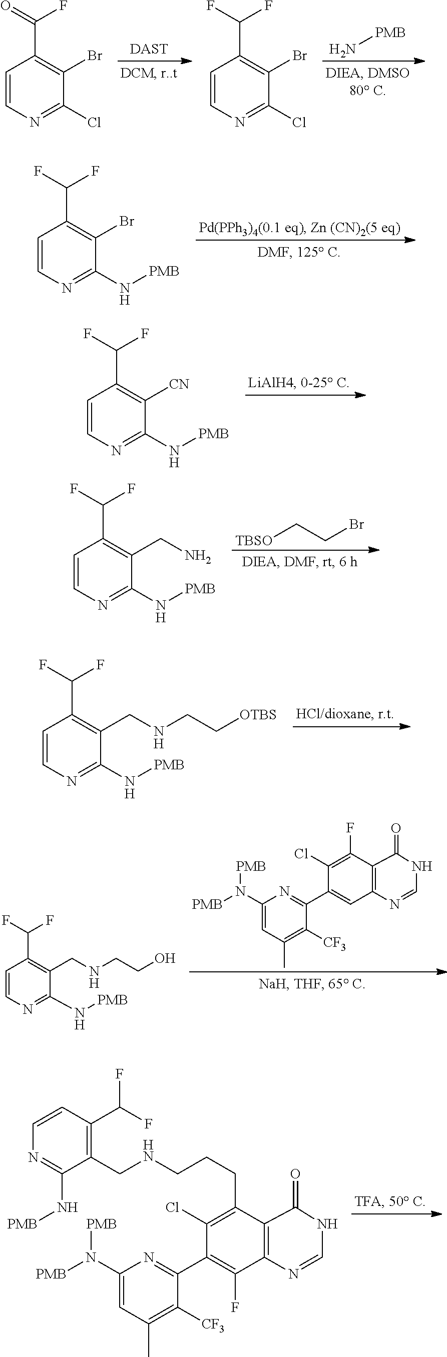

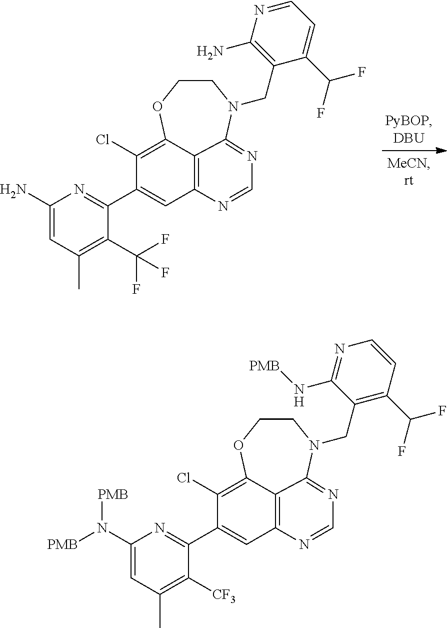

Step 1: 3-bromo-2-chloro-4-(difluoromethyl)pyridine

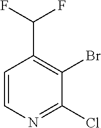 [SMILES: FC(F)c1ccnc(Cl)c1Br]

Under nitrogen, a solution of 3-bromo-2-chloro-pyridine-4-carbaldehyde (5.00 g, 22.68 mmol) in dichloromethane (50 mL) was added diethylamino trifluorosulfur (7.30 g, 45.34 mmol) at 0° C. The resulting solution was stirred at 25° C. for 2 hours. The reaction was quenched with saturated sodium bicarbonate aqueous solution. The reaction mixture was diluted with water. The resulting solution was extracted with dichloromethane and the organic layers were combined. The organic layer was dried over anhydrous sodium sulfate and concentrated under vacuum. The residue was purified by flash chromatography on silica gel eluting with petroleum ether/ethyl acetate (4/1) to afford 3-bromo-2-chloro-4-(difluoromethyl)pyridine (5.00 g, 20.62 mmol, 90.9% yield) as a white solid. LC-MS: (ESI, m/z): 243.9 [M+H]⁺

Step 2: 3-bromo-4-(difluoromethyl)-N-(4-methoxybenzyl)pyridin-2-amine

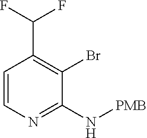

A solution of 3-bromo-2-chloro-4-(difluoromethyl)pyridine (850.0 mg, 3.51 mmol), 4-methoxybenzylamine (0.92 mL, 7.01 mmol) and N,N-diisopropylethylamine (1.35 g, 10.52 mmol) in dimethyl sulfoxide (5 mL) was stirred at 100° C. for 2 hours. After completion, the solvent was cooled to room temperature. The residue was purified by reverse chromatography on C18 gel eluting with acetonitrile/water (5%-95% in 30 min) to afford 3-bromo-4-(difluoromethyl)-N-(4-methoxybenzyl)pyridin-2-amine (800.0 mg, 2.33 mmol, 66.5% yield) as a yellow solid. LC-MS: (ESI, m/z): 345.1 [M+H]⁺

Step 3: 4-(difluoromethyl)-2-((4-methoxybenzyl)amino)nicotinonitrile

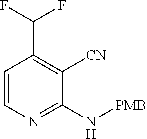

Under nitrogen, a solution of 3-bromo-4-(difluoromethyl)-N-(4-methoxybenzyl)pyridin-2-amine (3.00 g, 8.74 mmol), tetrakis(triphenylphosphine)palladium (1.20 g, 1.04 mmol) and zinc cyanide (5.10 g, 43.59 mmol) in N,N-dimethylformamide (25 mL) was added at 125° C. for 2 hours. After completion, the reaction was quenched with water. The reaction mixture was diluted with water. The resulting solution was extracted with acetate ethyl and the organic layers were combined. The organic layer was washed with water, dried over anhydrous sodium sulfate and concentrated under vacuum. The residue was purified by flash chromatography on silica gel eluting with petroleum ether/ethyl acetate (4/1) to afford 4-(difluoromethyl)-2-((4-methoxybenzyl)amino)nicotinonitrile (2.00 g, 6.91 mmol, 79.1% yield) as a yellow solid. LC-MS: (ESI, m/z): 290.1 [M+H]⁺

Step 4: 3-(aminomethyl)-4-(difluoromethyl)-N-(4-methoxybenzyl)pyridin-2-amine

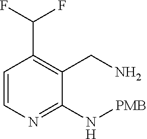

A solution of 4-(difluoromethyl)-2-((4-methoxybenzyl)amino)nicotinonitrile (300.0 mg, 1.04 mmol) and lithium aluminium hydride (120.0 mg, 3.16 mmol) in tetrahydrofuran (3 mL) was added at 0° C. and stirred at 0° C. for 2 hours. After completion, the reaction was quenched with water (0.1 ml). The reaction mixture was diluted with 15% sodium hydroxide solution (0.1 ml) and water (0.3 ml). After filtration, the filtrate was concentrated under reduced pressure. The residue was purified by reverse chromatography eluting with acetonitrile/water to afford 3-(aminomethyl)-4-(difluoromethyl)-N-(4-methoxybenzyl)pyridin-2-amine (80.0 mg, 0.27 mmol, 26.3% yield) as a yellow oil. LC-MS: (ESI, m/z): 294.1 [M+H]⁺

Step 5: 3-(((2-((tert-butyldimethylsilyl)oxy)ethyl)amino)methyl)-4-(difluoromethyl)-N-(4-methoxybenzyl)pyridin-2-amine

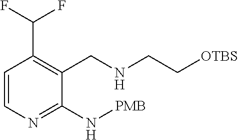

A solution of 3-(aminomethyl)-4-(difluoromethyl)-N-(4-methoxybenzyl)pyridin-2-amine (900.0 mg, 3.07 mmol), (2-bromoethoxy)-tert-butyldimethylsilane (0.97 mL, 4.51 mmol) and N,N-diisopropylethylamine (1.26 g, 9.75 mmol) in N,N-dimethylformamide (5 mL) was stirred at 90° C. for 2 hours. After completion, the reaction mixture was diluted with water. The resulting solution was extracted with ethyl acetate and the organic layers were combined. The organic layer was washed with water, dried over anhydrous sodium sulfate and concentrated under vacuum. The residue was purified by flash chromatography on silica gel eluting with dichloromethane/methanol (9/1) to afford 3-(((2-((tert-butyldimethylsilyl)oxy)ethyl)amino)methyl)-4-(difluoromethyl)-N-(4-methoxybenzyl)pyridin-2-amine (350.0 mg, 0.77 mmol, 25.3% yield) as a yellow oil. LC-MS: (ESI, m/z): 452.2 [M+H]⁺

Step 6: 2-(((4-(difluoromethyl)-2-((4-methoxybenzyl)amino)pyridin-3-yl)methyl)amino)ethan-1-ol

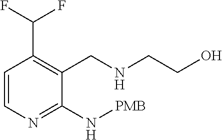

A solution of 3-(((2-((tert-butyldimethylsilyl)oxy)ethyl)amino)methyl)-4-(difluoromethyl)-N-(4-methoxybenzyl)pyridin-2-amine (300.0 mg, 0.66 mmol) in HCl/1,4-dioxane (3 mL, 12 mmol) was stirred at 25° C. for 3 hours. After completion, the solvent was concentrated under vacuum. The crude product would be directly used in the next step without purification. LC-MS: (ESI, m/z): 338.3 [M+H]⁺

Step 7: 7-(6-(bis(4-methoxybenzyl)amino)-4-methyl-3-(trifluoromethyl)pyridin-2-yl)-6-chloro-5-(2-(((4-(difluoromethyl)-2-((4-methoxybenzyl)amino)pyridin-3-yl)methyl)amino)ethoxy)quinazolin-4(3H)-one

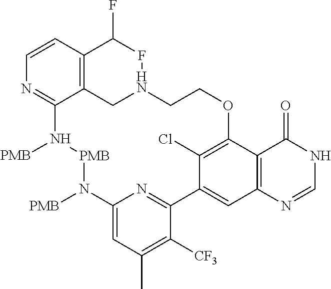

A solution of 7-(6-(bis(4-methoxybenzyl)amino)-4-methyl-3-(trifluoromethyl)pyridin-2-yl)-6-chloro-5-fluoroquinazolin-4(3H)-one (140.0 mg, 0.23 mmol) and 2-(((4-(difluoromethyl)-2-((4-methoxybenzyl)amino)pyridin-3-yl)methyl)amino)ethan-1-ol (100.0 mg, 0.30 mmol) in tetrahydrofuran (0.5 mL) was stirred at room temperature. Then sodium hydride (84.0 mg, 1.4 mmol, 60% dispersion in mineral oil) was added at 0° C. and stirred at 70° C. for 2 hours. After completion, the reaction was quenched with saturated ammonium chloride solution. The reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with acetonitrile/water (5-95% in 30 min) to afford 7-(6-(bis(4-methoxybenzyl)amino)-4-methyl-3-(trifluoromethyl)pyridin-2-yl)-6-chloro-5-(2-(((4-(difluoromethyl)-2-((4-methoxybenzyl)amino)pyridin-3-yl)methyl)amino)ethoxy)quinazolin-4(3H)-one (85.0 mg, 0.0914 mmol, 40% yield) as a yellow solid. LC-MS: (ESI, m/z): 930.3 [M+H]⁺

Step 8: 6-(8-chloro-4-((4-(difluoromethyl)-2-((4-methoxybenzyl)amino)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

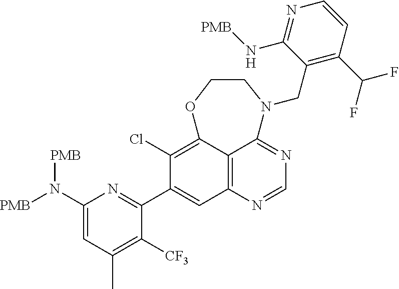

A solution of 7-(6-(bis(4-methoxybenzyl)amino)-4-methyl-3-(trifluoromethyl)pyridin-2-yl)-6-chloro-5-(2-(((4-(difluoromethyl)-2-((4-methoxybenzyl)amino)pyridin-3-yl)methyl)amino)ethoxy)quinazolin-4(3H)-one (300.0 mg, 0.32 mmol), benzotriazole-1-yl-oxytripyrrolidinophosphonium hexafluorophosphate (240.0 mg, 0.46 mmol) and 1,8-Diazabicyclo[5.4.0]undecane-7-ene (150.0 mg, 0.98 mmol) in acetonitrile (0.5 mL) was stirred at 25° C. for 2 hours. After completion, the reaction was quenched with water. The reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with acetonitrile/water (5-95% in 30 min) to afford 6-(8-chloro-4-((4-(difluoromethyl)-2-((4-methoxybenzyl)amino)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.21 mmol, 68% yield) as a yellow solid. LC-MS: (ESI, m/z): 912.3 [M+H]⁺

Step 9: 6-(4-((2-amino-4-(difluoromethyl)pyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine

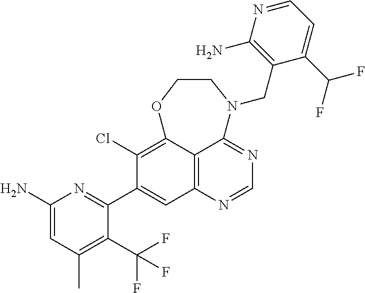 [SMILES: Cc1cc(N)nc(-c2cc3ncnc4c3c(c2Cl)OCCN4Cc2c(C(F)F)ccnc2N)c1C(F)(F)F]

A solution of 6-(8-chloro-4-((4-(difluoromethyl)-2-((4-methoxybenzyl)amino)pyridin-3-yl)methyl)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (100.0 mg, 0.11 mmol) in trifluoroacetic acid (2.0 mL) was stirred at 60° C. for 3 hours. After completion, the solvent was concentrated under vacuum. The product was purified by Prep-HPLC with the following conditions: Column: XBridge Prep OBD C18 Column, 19*250 mm, 5 μm; Mobile Phase A: Water (10 mmol/L NH4HCO3), Mobile Phase B: MeOH—Preparative; Flow rate: 25 mL/min; Gradient: 69% B to 78% B in 8 min, 78% B; Wave Length: 254 nm; RT1(min): 6.68; Number Of Runs: 0 to afford 6-(4-((2-amino-4-(difluoromethyl)pyridin-3-yl)methyl)-8-chloro-5,6-dihydro-4H-[1,4]oxazepino[5,6,7-de]quinazolin-9-yl)-4-methyl-5-(trifluoromethyl)pyridin-2-amine (42.5 mg, 0.07 mmol, 70.3% yield). LC-MS: (ESI, m/z): 552.2 [M+H]⁺

Example 57: ¹H NMR (400 MHz, DMSO-d₆) δ 8.54 (s, 1H), 8.13 (d, J=5.1 Hz, 1H), 7.48-7.14 (m, 2H), 6.87-6.67 (m, 3H), 6.43 (d, J=18.0 Hz, 3H), 5.11-5.06 (m, 2H), 4.57-4.46 (m, 2H), 3.68-3.34 (m, 2H), 2.36 (d, J=2.3 Hz, 3H)

Example 58: (R)-3-(1-(9-(6-amino-4-methyl-3-(trifluoromethyl)pyridin-2-yl)-8-chloro-5,6-dihydro-4H-[1,4]oxazepino[5,6,7-de]quinazolin-4-yl)ethyl)-N5-methylpyridine-2,5-diamine

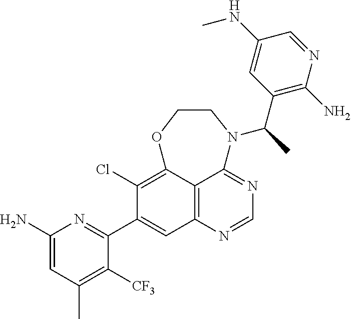 [SMILES: CNc1cnc(N)c([C@@H](C)N2CCOc3c(Cl)c(-c4nc(N)cc(C)c4C(F)(F)F)cc4ncnc2c34)c1]

### Synthetic Route

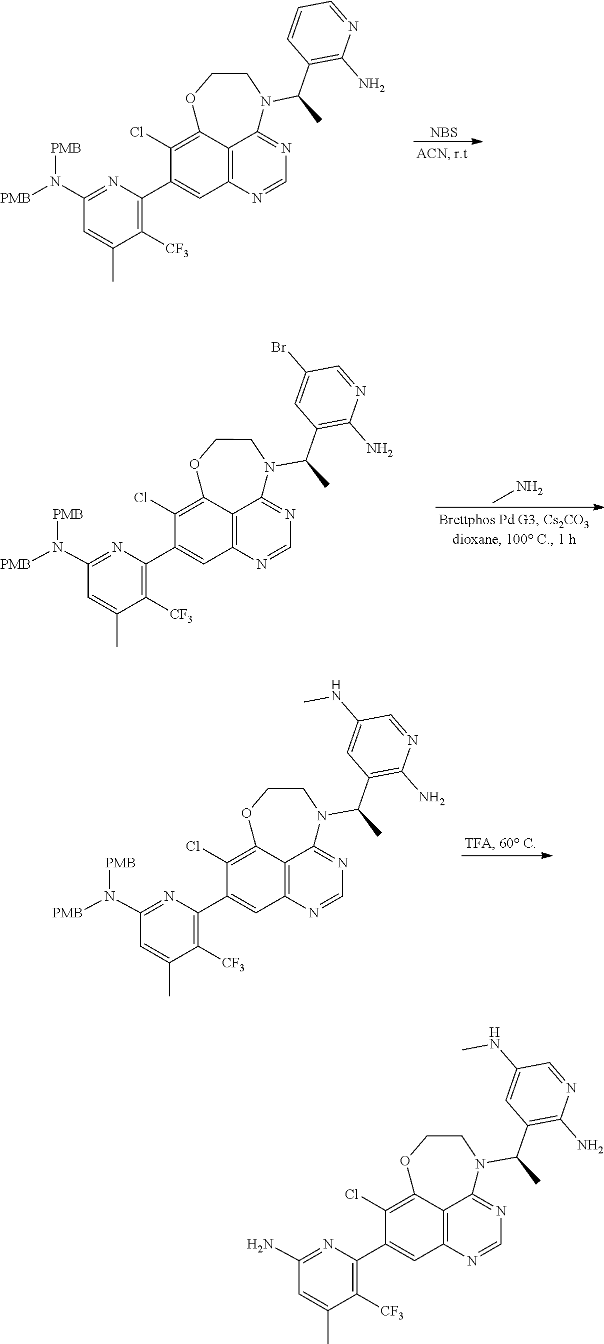

Step 1: (R)-6-(4-(I-(2-amino-5-bromopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

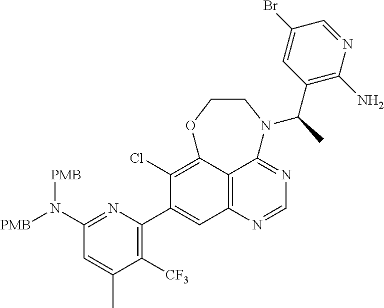

To a mixture of (R)-6-(4-(1-(2-aminopyridin-3-yl)ethyl)-8-chloro-5,6-dihydro-4H-[1,4]oxazepino[5,6,7-de]quinazolin-9-yl)-N, N-bis(4-methoxybenzyl)-4-methyl-5-(trifluoromethyl)pyridin-2-amine (270.0 mg, 0.36 mmol) in acetonitrile (1 mL) was added 1-bromo-2,5-pyrrolidinedione (68.0 mg, 0.38 mmol) and stirred for 1 hour at room temperature. After completion, the resulting solution was diluted with ethyl acetate, washed with brine, dried over sodium sulfate and concentrated. The residue was purified by flash chromatography on silica gel eluting with petroleum ether/ethyl acetate (1/4). This resulted in (R)-6-(4-(1-(2-amino-5-bromopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (260.0 mg, 0.31 mmol, 87.2% yield) as a yellow solid. LCMS (ESI, m/z): 834.2 [M+H]⁺.

Step 4: (R)-3-(1-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)ethyl)-N5-methylpyridine-2,5-diamine

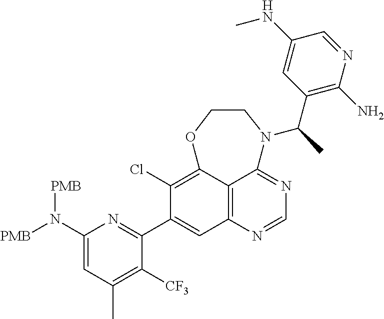

A mixture of (R)-6-(4-(1-(2-amino-5-bromopyridin-3-yl)ethyl)-8-chloro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (110.0 mg, 0.13 mmol), cesium carbonate (130.0 mg, 0.4 mmol), BrettPhos Pd G3 (15.0 mg, 0.02 mmol) and methanamine (0.6 mL, 1.2 mmol) in 1,4-dioxane (5 mL) was stirred at 100° C. for 1 hour under nitrogen. After completion, the organic layer was concentrated in vacuum. The residue was purified by flash chromatography on silica gel eluting with dichloromethane/methanol (47/3). This resulted in (R)-3-(1-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)ethyl)-N5-methylpyridine-2,5-diamine (90.0 mg, 0.11 mmol, 87% yield) as a yellow solid. LCMS (ESI, m/z): 785.3 [M+H]⁺.

Step 3: (R)-3-(1-(9-(6-amino-4-methyl-3-(trifluoromethyl)pyridin-2-yl)-8-chloro-5,6-dihydro-4H-[1,4]oxazepino[5,6,7-de]quinazolin-4-yl)ethyl)-N5-methylpyridine-2,5-diamine

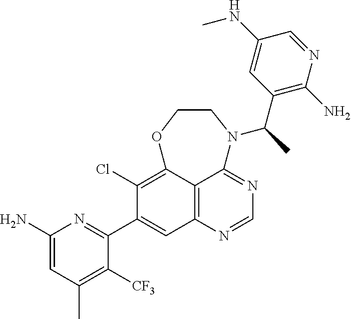 [SMILES: CNc1cnc(N)c([C@@H](C)N2CCOc3c(Cl)c(-c4nc(N)cc(C)c4C(F)(F)F)cc4ncnc2c34)c1]

A mixture of (R)-3-(1-(9-(6-(bis(4-methoxybenzyl)amino)-4-methyl-3-(trifluoromethyl)pyridin-2-yl)-8-chloro-5,6-dihydro-4H-[1,4]oxazepino[5,6,7-de]quinazolin-4-yl)ethyl)-N5-methylpyridine-2,5-diamine (90.0 mg, 0.11 mmol) in 2,2,2-trifluoroacetic acid (3 mL) was stirred at 60° C. for 3 hours. After completion, the mixture was concentrated under vacuum. The crude product was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 29% B to 59% B in 7 min; Wave Length: 254 nm; RT1(min): 6.5. This resulted in 3-[(1R)-1-[7-[6-amino-4-methyl-3-(trifluoromethyl)-2-pyridyl]-8-chloro-10-oxa-2,4,13-triazatricyclo[7.4.1.05,14]tetradeca-1,3,5(14),6,8-pentaen-13-yl]ethyl]-N5-methyl-pyridine-2,5-diamine (14.6 mg, 0.027 mmol, 23.4% yield). LCMS (ESI, m/z): 545.25 [M+H]⁺.

Example 58: ¹H NMR (400 MHz, Methanol-d₄) δ 8.51 (s, 1H), 7.46 (dd, J=2.8, 1.7 Hz, 1H), 7.27 (d, J=1.0 Hz, 1H), 7.26-7.24 (m, 1H), 6.76-6.65 (m, 1H), 6.58 (s, 1H), 4.56-4.46 (m, 1H), 4.39-4.29 (m, 1H), 3.75-3.64 (m, 1H), 3.61-3.51 (m, 1H), 2.80 (s, 3H), 2.44 (d, J=1.6 Hz, 3H), 1.63 (d, J=6.8 Hz, 3H).

Example 59: (S)-4-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)naphthalen-2-ol

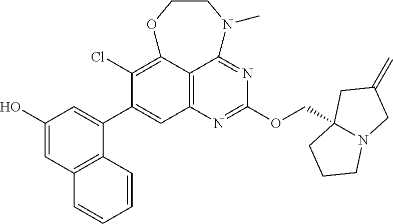 [SMILES: C=C1CN2CCC[C@@]2(COc2nc3c4c(c(Cl)c(-c5cc(O)cc6ccccc56)cc4n2)OCCN3C)C1]

### Synthetic Route

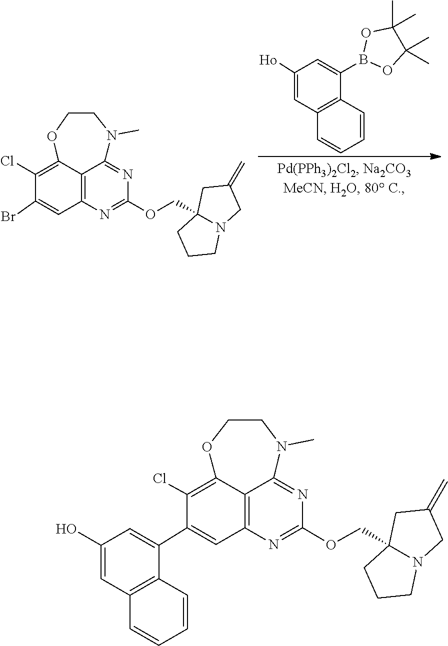

Step 1: (S)-4-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)naphthalen-2-ol

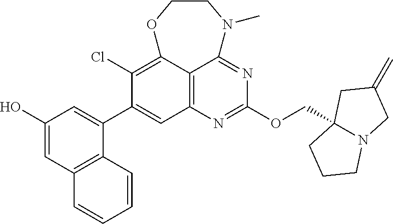 [SMILES: C=C1CN2CCC[C@@]2(COc2nc3c4c(c(Cl)c(-c5cc(O)cc6ccccc56)cc4n2)OCCN3C)C1]

A solution of (S)-9-bromo-8-chloro-4-methyl-2-((2-methylenetetrahydro-1H-pyrrolizin-7a(5H)-yl)methoxy)-5,6-dihydro-4H-[1,4]oxazepino[5,6,7-de]quinazoline (50.0 mg, 0.107 mmol), 4-(4,4,5,5-tetramethyl-1,3,2-dioxaborolan-2-yl)naphthalen-2-ol (58.0 mg, 0.215 mmol), bis(triphenylphosphine)palladium(II) dichloride (7.5 mg, 0.0107 mmol), and sodium carbonate (45.5 mg, 0.429 mmol) in acetonitrile (2.15 mL) and water (0.54 mL) was stirred at 80° C. for 60 minutes under nitrogen. After completion, the reaction mixture was cooled to room temperature and concentrated in vacuo. The residue was dissolved in DCM, filtered to remove solids, and concentrated in vacuo. The crude material was purified by Prep-HPLC with the following conditions: Column: XSelect CSH Prep C18, 50×30 mm, 5 μm; Mobile Phase A: 0.1% ammonium hydroxide in water, Mobile Phase B: acetonitrile; Flow rate: 60 mL/min; Gradient: 40% B to 80% B over 10 min; wavelength: 240 nm; column temp: 25° C. This afforded (S)-4-(8-chloro-4-methyl-2-((2-methylenetetrahydro-1H-pyrrolizin-7a(5H)-yl)methoxy)-5,6-dihydro-4H-[1,4]oxazepino[5,6,7-de]quinazolin-9-yl)naphthalen-2-ol (20.5 mg, 36% yield). LC-MS: (ESI, m/z): 529.1 [M+H]⁺

Example 59: ¹H NMR (400 MHz, DMSO-d₆) δ 9.91 (s, 1H), 7.76 (dd, J=8.3, 1.1 Hz, 1H), 7.40 (ddd, J=8.2, 6.3, 1.7 Hz, 1H), 7.31-7.11 (m, 3H), 7.07 (s, 1H), 6.97 (d, J=2.4 Hz, 1H), 4.92-4.86 (m, 2H), 4.72-4.55 (m, 2H), 4.07-3.88 (m, 4H), 3.58-3.50 (m, 1H), 3.31 (s, 3H), 3.21-3.14 (m, 1H), 3.05-2.92 (m, 1H), 2.64-2.50 (m, 2H), 2.38-2.30 (m, 1H), 2.02-1.57 (m, 4H).

Example 60: 6-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

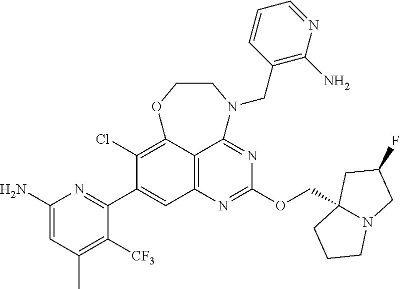 [SMILES: Cc1cc(N)nc(-c2cc3nc(OC[C@@]45CCCN4C[C@H](F)C5)nc4c3c(c2Cl)OCCN4Cc2cccnc2N)c1C(F)(F)F]

### Synthetic Route

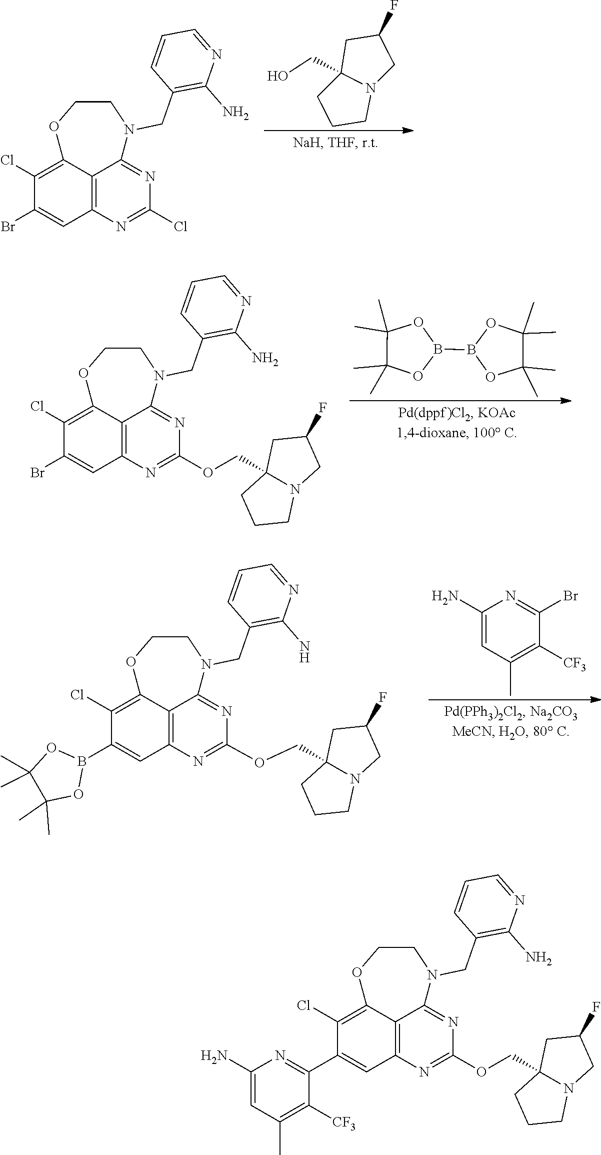

Step 1: 3-((9-bromo-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine

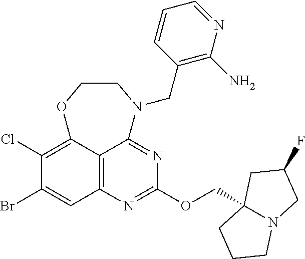 [SMILES: Nc1ncccc1CN1CCOc2c(Cl)c(Br)cc3nc(OC[C@@]45CCCN4C[C@H](F)C5)nc1c23]

A solution of ((2R,7aS)-2-fluorotetrahydro-1H-pyrrolizin-7a(5H)-yl)methanol (649.6 mg, 4.08 mmol) in tetrahydrofuran (10 mL) was added sodium hydride (326.4 mg, 8.16 mmol, 60% purity) at 0° C. and stirred at 0° C. for 30 min. Then 3-((9-bromo-2,8-dichloro-5,6-dihydro-4H-[1,4]oxazepino[5,6,7-de]quinazolin-4-yl)methyl)pyridin-2-amine (900.0 mg, 2.04 mmol) was added and stirred at 25° C. for 3 hours. After completion, the reaction was quenched with ammonium chloride saturated solution, diluted with dichloromethane, washed with water, dried over anhydrous sodium sulfate and concentrated under vacuum. The residue was purified by flash chromatography on silica gel eluting with dichloromethane/ethyl acetate (7/3) to afford 3-((9-bromo-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine (422.1 mg, 0.67 mmol, 33% yield) as a white solid. LC-MS: (ESI, m/z): 563.1 [M+H]⁺

Step 2: 3-((8-chloro-2-(((2R,7aS)-2-fluorotetrahydro-1H-pyrrolizin-7a(5H)-yl)methoxy)-9-(4,4,5,5-tetramethyl-1,3,2-dioxaborolan-2-yl)-5,6-dihydro-4H-[1,4]oxazepino[5,6,7-de]quinazolin-4-yl)methyl)pyridin-2-amine

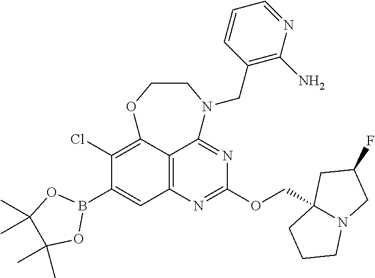 [SMILES: CC1(C)OB(c2cc3nc(OC[C@@]45CCCN4C[C@H](F)C5)nc4c3c(c2Cl)OCCN4Cc2cccnc2N)OC1(C)C]

A solution of 3-((9-bromo-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine (50.0 mg, 0.089 mmol), bis(pinacolato)diboron (45.0 mg, 0.177 mmol), 1,1′-bis(diphenylphosphino)ferrocene-palladium(II)dichloride (6.5 mg, 0.0089 mmol), and potassium acetate (17.4 mg, 0.177 mmol) in 1,4-dioxane (0.9 mL) was stirred at 100° C. for 3.5 hours under nitrogen. After completion, the reaction mixture was cooled to room temperature and concentrated in vacuo. The residue was dissolved in DCM, filtered to remove solids, and concentrated in vacuo to afford a brown oil which was used directly in the next step without purification (40 mg crude).

Step 3: 6-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

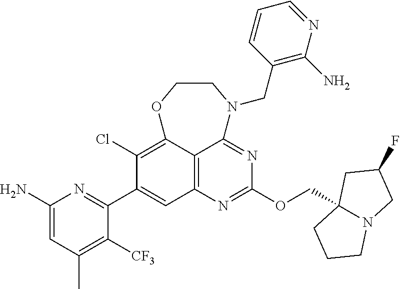 [SMILES: Cc1cc(N)nc(-c2cc3nc(OC[C@@]45CCCN4C[C@H](F)C5)nc4c3c(c2Cl)OCCN4Cc2cccnc2N)c1C(F)(F)F]

A solution of (3-((8-chloro-2-(((2R,7aS)-2-fluorotetrahydro-1H-pyrrolizin-7a(5H)-yl)methoxy)-9-(4,4,5,5-tetramethyl-1,3,2-dioxaborolan-2-yl)-5,6-dihydro-4H-[1,4]oxazepino[5,6,7-de]quinazolin-4-yl)methyl)pyridin-2-amine (40.0 mg, 0.065 mmol), 6-bromo-4-methyl-5-(trifluoromethyl)pyridine-2-amine (38.6 mg, 0.15 mmol), bis(triphenylphosphine)palladium(II) dichloride (5.3 mg, 0.0076 mmol), and sodium carbonate (32.1 mg, 0.303 mmol) in acetonitrile (1.5 mL) and water (0.38 mL) was stirred at 80° C. for 60 minutes under nitrogen. After completion, the reaction mixture was cooled to room temperature and concentrated in vacuo. The residue was dissolved in DCM, filtered to remove solids, and concentrated in vacuo. The crude material was purified by Prep-HPLC with the following conditions: Column: XSelect CSH Prep C18, 50×30 mm, 5 μm; Mobile Phase A: 0.1% formic acid in water, Mobile Phase B: acetonitrile; Flow rate: 60 mL/min; Gradient: 5% B to 50% B over 10 min; wavelength: 240 nm; column temp: 25° C. This afforded 6-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (7.4 mg, 15% yield). LC-MS: (ESI, m/z): 659.2 [M+H]⁺

Example 60: ¹H NMR (400 MHz, DMSO-d6) δ 7.89 (dd, J=4.9, 1.8 Hz, 1H), 7.28 (dd, J=7.4, 1.8 Hz, 1H), 6.95 (s, 1H), 6.72 (s, 2H), 6.52 (dd, J=7.3, 4.9 Hz, 1H), 6.44 (s, 1H), 6.03 (s, 2H), 5.20 (d, J=53.7 Hz, 1H), 4.89-4.76 (m, 2H), 4.68-4.54 (m, 2H), 4.09-3.76 (m, 4H), 3.07-2.92 (m, 2H), 2.82-2.65 (m, 2H), 2.35 (d, J=2.2 Hz, 3H), 2.13-1.57 (m, 6H).

Example 61: 4-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)naphthalen-2-ol

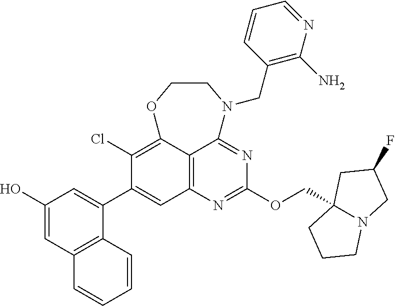 [SMILES: Nc1ncccc1CN1CCOc2c(Cl)c(-c3cc(O)cc4ccccc34)cc3nc(OC[C@@]45CCCN4C[C@H](F)C5)nc1c23]

### Synthetic Route

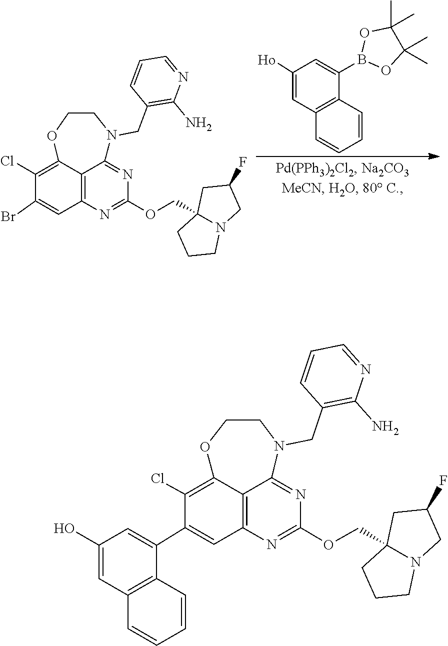

Step 1: 4-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)naphthalen-2-ol

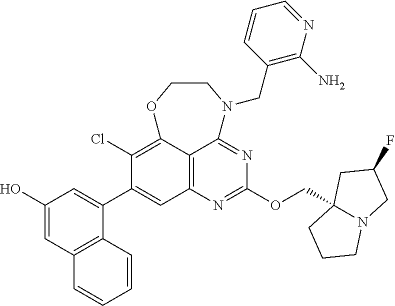 [SMILES: Nc1ncccc1CN1CCOc2c(Cl)c(-c3cc(O)cc4ccccc34)cc3nc(OC[C@@]45CCCN4C[C@H](F)C5)nc1c23]

A solution of 3-((9-bromo-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine (40 mg, 0.071 mmol), 4-(4,4,5,5-tetramethyl-1,3,2-dioxaborolan-2-yl)naphthalen-2-ol (38 mg, 0.14 mmol), bis(triphenylphosphine)palladium(II) dichloride (5.0 mg, 0.0071 mmol), and sodium carbonate (30 mg, 0.28 mmol) in acetonitrile (1.4 mL) and water (0.35 mL) was stirred at 80° C. for 60 minutes under nitrogen. After completion, the reaction mixture was cooled to room temperature and concentrated in vacuo. The residue was dissolved in DCM, filtered to remove solids, and concentrated in vacuo. The crude material was purified by SFC with the following conditions: Column: Synergi Polar RP, 150×21.2 mm, 5 μm; Mobile Phase: 0.1% ammonium hydroxide in methanol; Flow rate: 70 mL/min; Gradient: isocratic 30% over 5 min; wavelength: 225 nm; column temp: 40° C. This afforded 4-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)naphthalen-2-ol (26.0 mg, 58% yield). LC-MS: (ESI, m/z): 627.1 [M+H]⁺

Example 61: ¹H NMR (400 MHz, DMSO-d₆) δ 9.93 (s, 1H), 7.91 (dd, J=4.9, 1.8 Hz, 1H), 7.84-7.67 (m, 1H), 7.41 (ddd, J=8.2, 6.5, 1.5 Hz, 1H), 7.31 (dd, J=7.4, 1.8 Hz, 1H), 7.28-7.16 (m, 3H), 7.11 (s, 1H), 6.99-6.96 (m, 1H), 6.54 (dd, J=7.3, 4.9 Hz, 1H), 6.04 (s, 2H), 5.19 (d, J=53.1 Hz, 1H), 5.02-4.76 (m, 2H), 4.76-4.53 (m, 2H), 4.09-3.79 (m, 4H), 3.11-2.69 (m, 4H), 2.14-1.60 (m, 6H).

Example 62: 3-((8-chloro-2-(((2R,7aS)-2-fluorotetrahydro-1H-pyrrolizin-7a(5H)-yl)methoxy)-9-(naphthalen-1-yl)-5,6-dihydro-4H-[1,4]oxazepino[5,6,7-de]quinazolin-4-yl)methyl)pyridin-2-amine

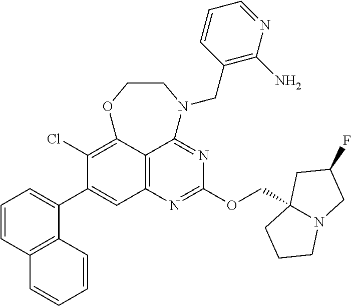 [SMILES: Nc1ncccc1CN1CCOc2c(Cl)c(-c3cccc4ccccc34)cc3nc(OC[C@@]45CCCN4C[C@H](F)C5)nc1c23]

### Synthetic Route

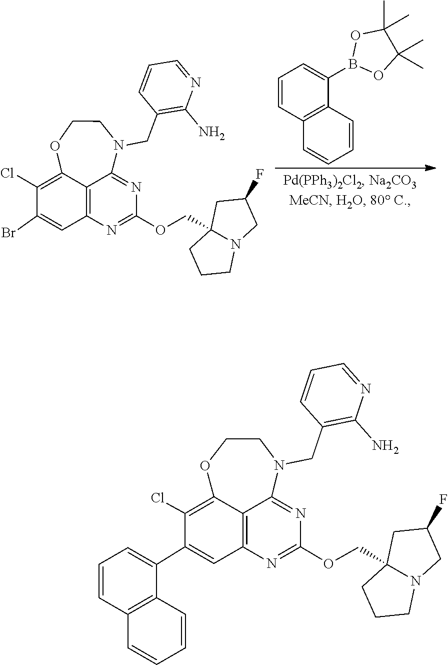 [SMILES: Nc1ncccc1CN1CCOc2c(Cl)c(Br)cc3nc(OC[C@@]45CCCN4C[C@H](F)C5)nc1c23 ; CC1(C)OB(c2cccc3ccccc23)OC1(C)C ; Nc1ncccc1CN1CCOc2c(Cl)c(-c3cccc4ccccc34)cc3nc(OC[C@@]45CCCN4C[C@H](F)C5)nc1c23]

Step 1: 3-((8-chloro-2-(((2R,7aS)-2-fluorotetrahydro-1H-pyrrolizin-7a(5H)-yl)methoxy)-9-(naphthalen-1-yl)-5,6-dihydro-4H-[1,4]oxazepino[5,6,7-de]quinazolin-4-yl)methyl)pyridin-2-amine

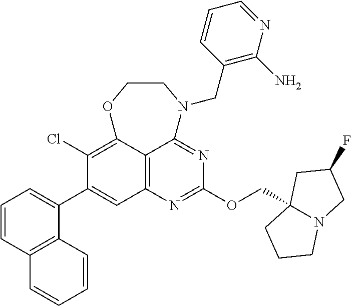 [SMILES: Nc1ncccc1CN1CCOc2c(Cl)c(-c3cccc4ccccc34)cc3nc(OC[C@@]45CCCN4C[C@H](F)C5)nc1c23]

A solution of 3-((9-bromo-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine (40 mg, 0.071 mmol), 4,4,5,5-tetramethyl-2-(naphthalen-1-yl)-1,3,2-dioxaborolane (36 mg, 0.14 mmol), bis(triphenylphosphine)palladium(II) dichloride (5.0 mg, 0.0071 mmol), and sodium carbonate (30 mg, 0.28 mmol) in acetonitrile (1.4 mL) and water (0.35 mL) was stirred at 80° C. for 60 minutes under nitrogen. After completion, the reaction mixture was cooled to room temperature and concentrated in vacuo. The residue was dissolved in DCM, filtered to remove solids, and concentrated in vacuo. The crude material was purified by Prep-HPLC with the following conditions: Column: XSelect CSH Prep C18, 50×30 mm, 5 μm; Mobile Phase A: 0.1% ammonium hydroxide in water, Mobile Phase B: acetonitrile; Flow rate: 60 mL/min; Gradient: 40% B to 80% B over 10 min; wavelength: 222 nm; column temp: 25° C. This afforded 3-((8-chloro-2-(((2R,7aS)-2-fluorotetrahydro-1H-pyrrolizin-7a(5H)-yl)methoxy)-9-(naphthalen-1-yl)-5,6-dihydro-4H-[1,4]oxazepino[5,6,7-de]quinazolin-4-yl)methyl)pyridin-2-amine (31.7 mg, 73% yield). LC-MS: (ESI, m/z): 611.2 [M+H]⁺

Example 62: ¹H NMR (400 MHz, DMSO-d₆) δ 8.13-7.96 (m, 2H), 7.91 (dd, J=4.9, 1.8 Hz, 1H), 7.62 (dd, J=8.3, 7.0 Hz, 1H), 7.55 (ddd, J=8.2, 6.6, 1.5 Hz, 1H), 7.51-7.38 (m, 3H), 7.31 (dd, J=7.4, 1.8 Hz, 1H), 7.14 (s, 1H), 6.54 (dd, J=7.3, 5.0 Hz, 1H), 6.04 (s, 2H), 5.19 (d, J=54.7 Hz, 1H), 4.99-4.77 (m, 2H), 4.77-4.54 (m, 2H), 4.03-3.78 (m, 4H), 3.10-2.70 (m, 4H), 2.17-1.46 (m, 6H).

Example 63: 3-((8-chloro-9-(2,3-dihydro-1H-inden-4-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine

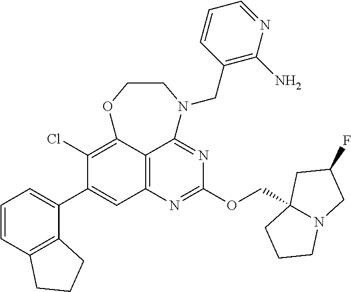 [SMILES: Nc1ncccc1CN1CCOc2c(Cl)c(-c3cccc4c3CCC4)cc3nc(OC[C@@]45CCCN4C[C@H](F)C5)nc1c23]

### Synthetic Route

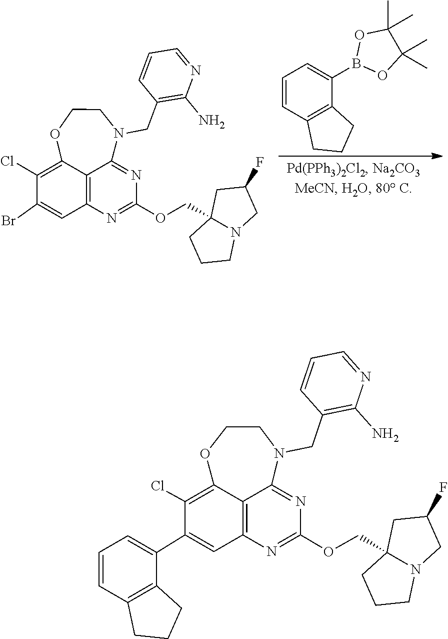 [SMILES: Nc1ncccc1CN1CCOc2c(Cl)c(Br)cc3nc(OC[C@@]45CCCN4C[C@H](F)C5)nc1c23 ; CC1(C)OB(c2cccc3c2CCC3)OC1(C)C ; Nc1ncccc1CN1CCOc2c(Cl)c(-c3cccc4c3CCC4)cc3nc(OC[C@@]45CCCN4C[C@H](F)C5)nc1c23]

Step 1: 3-((8-chloro-9-(2,3-dihydro-1H-inden-4-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine

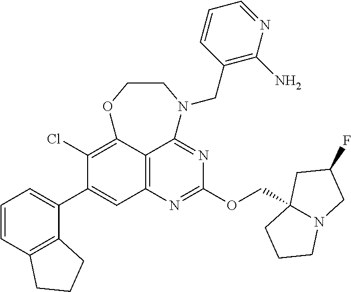 [SMILES: Nc1ncccc1CN1CCOc2c(Cl)c(-c3cccc4c3CCC4)cc3nc(OC[C@@]45CCCN4C[C@H](F)C5)nc1c23]

A solution of 3-((9-bromo-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine (40 mg, 0.071 mmol), 2-(2,3-dihydro-1H-inden-4-yl)-4,4,5,5-tetramethyl-1,3,2-dioxaborolane (34.6 mg, 0.14 mmol), bis(triphenylphosphine)palladium(II) dichloride (5.0 mg, 0.0071 mmol), and sodium carbonate (30 mg, 0.28 mmol) in acetonitrile (1.4 mL) and water (0.35 mL) was stirred at 80° C. for 1.5 hours under nitrogen. After completion, the reaction mixture was cooled to room temperature and concentrated in vacuo. The residue was dissolved in DCM, filtered to remove solids, and concentrated in vacuo. The crude material was purified by Prep-HPLC with the following conditions: Column: XSelect CSH Prep C18, 50×30 mm, 5 μm; Mobile Phase A: 0.1% formic acid in water, Mobile Phase B: acetonitrile; Flow rate: 60 mL/min; Gradient: 5% B to 50% B over 10 min; wavelength: 235 nm; column temp: 25° C. This afforded 3-((8-chloro-9-(2,3-dihydro-1H-inden-4-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine (40 mg, 94% yield). LC-MS: (ESI, m/z): 601.2 [M+H]⁺

Example 63: ¹H NMR (400 MHz, DMSO-de) b 7.89 (dd, J=4.9, 1.8 Hz, 1H), 7.33-7.26 (m, 2H), 7.23 (t, J=7.5 Hz, 1H), 7.03 (d, J=6.3 Hz, 2H), 6.52 (dd, J=7.3, 4.9 Hz, 1H), 6.02 (s, 2H), 5.20 (d, J=52.6 Hz, 1H), 4.93-4.77 (m, 2H), 4.68-4.59 (m, 2H), 4.08-3.78 (m, 4H), 3.11-2.88 (m, 5H), 2.85-2.59 (m, 3H), 2.12-1.59 (m, 8H).

Example 64: (3-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)phenyl)methanol

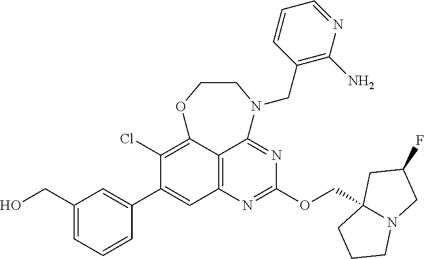 [SMILES: Nc1ncccc1CN1CCOc2c(Cl)c(-c3cccc(CO)c3)cc3nc(OC[C@@]45CCCN4C[C@H](F)C5)nc1c23]

### Synthetic Route

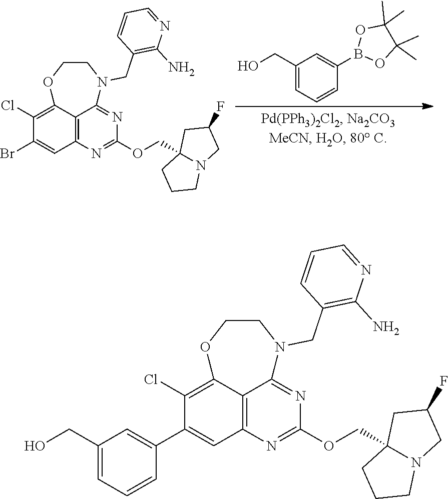 [SMILES: Nc1ncccc1CN1CCOc2c(Cl)c(Br)cc3nc(OC[C@@]45CCCN4C[C@H](F)C5)nc1c23 ; CC1(C)OB(c2cccc(CO)c2)OC1(C)C ; Nc1ncccc1CN1CCOc2c(Cl)c(-c3cccc(CO)c3)cc3nc(OC[C@@]45CCCN4C[C@H](F)C5)nc1c23]

Step 1: (3-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)phenyl)methanol

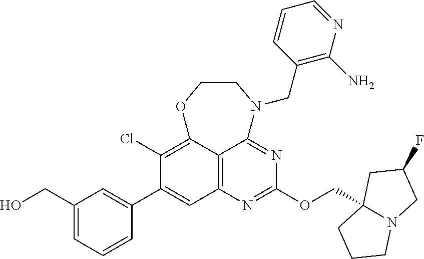 [SMILES: Nc1ncccc1CN1CCOc2c(Cl)c(-c3cccc(CO)c3)cc3nc(OC[C@@]45CCCN4C[C@H](F)C5)nc1c23]

A solution of 3-((9-bromo-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)methyl)pyridin-2-amine (40 mg, 0.071 mmol), (3-(4,4,5,5-tetramethyl-1,3,2-dioxaborolan-2-yl)phenyl)methanol (33 mg, 0.14 mmol), bis(triphenylphosphine)palladium(II) dichloride (5.0 mg, 0.0071 mmol), and sodium carbonate (30 mg, 0.28 mmol) in acetonitrile (1.4 mL) and water (0.35 mL) was stirred at 80° C. for 60 minutes under nitrogen. After completion, the reaction mixture was cooled to room temperature and concentrated in vacuo. The residue was dissolved in DCM, filtered to remove solids, and concentrated in vacuo. The crude material was purified by Prep-HPLC with the following conditions: Column: XSelect CSH Prep C18, 50×30 mm, 5 μm; Mobile Phase A: 0.1% ammonium hydroxide in water, Mobile Phase B: acetonitrile; Flow rate: 60 mL/min; Gradient: 20% B to 60% B over 10 min; wavelength: 254 nm; column temp: 25° C. This afforded (3-(4-((2-aminopyridin-3-yl)methyl)-8-chloro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)phenyl)methanol (29.9 mg, 71% yield). LC-MS: (ESI, m/z): 591.2 [M+H]⁺

Example 64: ¹H NMR (400 MHz, DMSO-d₆) δ 7.89 (dd, J=4.9, 1.8 Hz, 1H), 7.48-7.31 (m, 4H), 7.27 (dd. J=7.4, 1.8 Hz, 1H), 7.11 (s, 1H), 6.52 (dd, J=7.3, 4.9 Hz, 1H), 6.03 (s, 2H), 5.30-5.11 (m, 2H), 4.92-4.76 (m, 2H), 4.64 (dd, J=5.7, 2.8 Hz, 2H), 4.57 (d, J=5.7 Hz, 2H), 3.96 (q, J=10.4 Hz, 2H), 3.91-3.84 (m, 2H), 3.08-2.86 (m, 3H), 2.83-2.73 (m, 1H), 2.06-1.62 (m, 6H).

Example 65a & 65b: (R)-8-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine & (R)-8-((S)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine

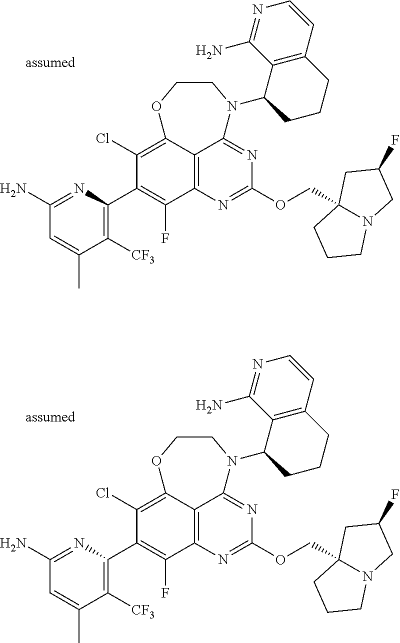

### Synthetic Route

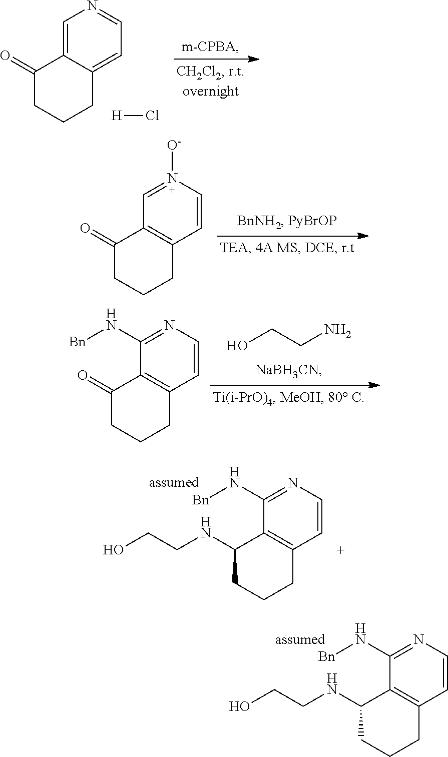

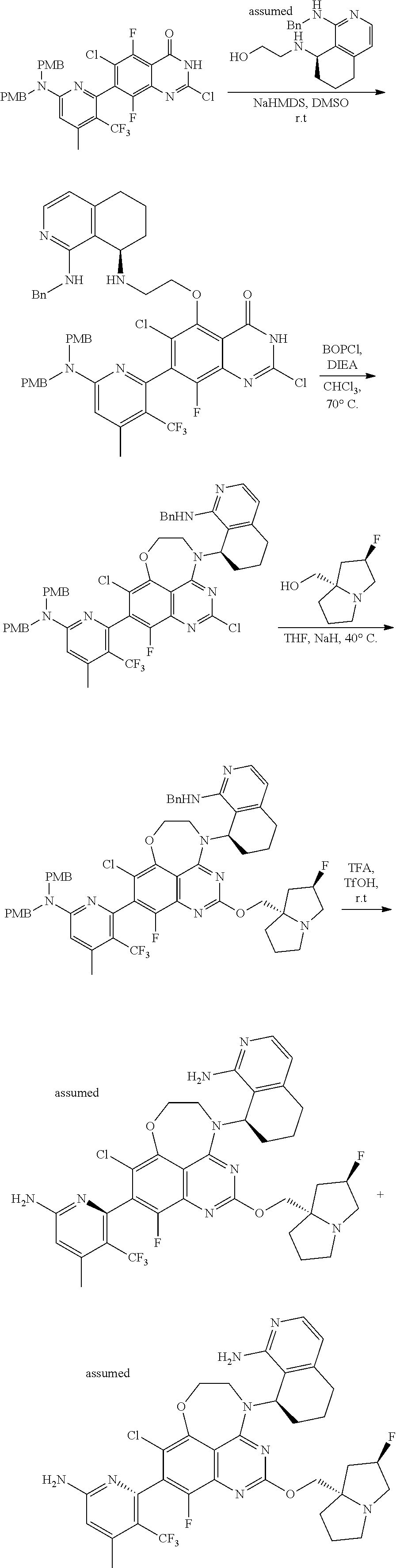

Step 1: 8-oxo-5,6,7,8-tetrahydroisoquinoline 2-oxide

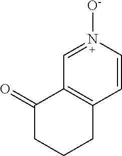

A solution of 6,7-dihydroisoquinolin-8(5H)-one hydrochloride (46.80 g, 254.88 mmol) and 3-chloroperoxybenzoic acid (74.76 g, 433.26 mmol) in dichloromethane (500 mL) was stirred at 25° C. for 18 hours. After completion, the resulting mixture quenched with saturated sodium sulfite and concentrated under vacuum. The residue was purified by flash chromatography on silica gel eluting with dichloromethane/methanol (1:1) to afford 8-oxo-5,6,7,8-tetrahydroisoquinoline 2-oxide (19.80 g, 116.52 mmol, 45.7% yield) as a brown oil. LC-MS: (ESI, m/z): 164.1 [M+H]⁺

Step 2: 1-(benzylamino)-6,7-dihydroisoquinolin-8(5H)-one

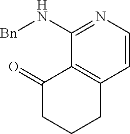 [SMILES: O=C1CCCc2ccnc(NCc3ccccc3)c21]

Triethylamine (29.02 g, 286.80 mmol), 4A molecular sieves (of the same mass as 8-oxo-5,6,7,8-tetrahydroisoquinoline 2-oxide (15.60 g)) and bromo[tri(1-pyrrolidinyl)]phosphonium hexafluorophosphate (57.94 g, 124.26 mmol) were sequentially added to a solution of 8-oxo-5,6,7,8-tetrahydroisoquinoline 2-oxide (15.60 g, 95.58 mmol) and benzylamine (25.61 g, 239.04 mmol) in dry 1,2-dichloroethane (500 mL). The resulting mixture was stirred at 25° C. for 1.5 hours. After completion, the reaction mixture was filtered, and the mother liquor was diluted with saturated ammonium chloride solution and extracted with dichloromethane. The combined organic phase was dried over anhydrous sodium sulfate, concentrated under vacuum and purified by flash chromatography on silica gel eluting with petroleum ether/ethyl acetate (95:5) to afford 1-(benzylamino)-6,7-dihydroisoquinolin-8(5H)-one (7.20 g, 28.26 mmol, 29.5% yield) as a light yellow solid. LC-MS: (ESI, m/z): 253.1 [M+H]⁺

Step 3: (R)-2-((1-(benzylamino)-5,6,7,8-tetrahydroisoquinolin-8-yl)amino)ethan-1-ol & (S)-2-((1-(benzylamino)-5,6,7,8-tetrahydroisoquinolin-8-yl)amino)ethan-1-ol

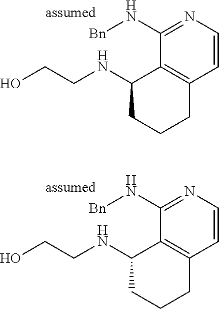 [SMILES: OCCN[C@@H]1CCCc2ccnc(NCc3ccccc3)c21 ; OCCN[C@H]1CCCc2ccnc(NCc3ccccc3)c21]

A solution of 1-(benzylamino)-6,7-dihydroisoquinolin-8(5H)-one (5.80 g, 22.99 mmol), 2-aminoethan-1-ol (3.51 g, 57.47 mmol) and titanium(IV)isopropoxide (20 mL, 67.55 mmol) in methyl alcohol (80 mL) was stirred at 25° C. for 30 min. Then sodiumcyanoborohydride (4.33 g, 68.96 mmol) was added and stirred at 80° C. for 67 hours. After completion, the reaction mixture was quenched with water and concentrated under vacuum. The residue was purified by flash chromatography on silica gel eluting with dichloromethane/methanol (96:4) to afford product. The product was further purified by Chiral-Prep-HPLC with following conditions: Column: CHIRAL ART Cellulose-SB, 7*25 cm, 10 μm; Mobile Phase A: CO₂ Mobile Phase B: methanol (0.1% 2M NH₃-MEOH); Flow rate: 250 mL/min; Gradient: isocratic 35% B; Column Temperature (° C.): 35; Back Pressure (bar): 100; Wave Length: 254 nm; RT1(min): 7.03; RT2(min): 8.08; Sample Solvent: methanol—Preparative; Injection Volume: 3 mL; Number Of Runs: 13 to afford (R)-2-((1-(benzylamino)-5,6,7,8-tetrahydroisoquinolin-8-yl)amino)ethan-1-ol (1.30 g, 4.07 mmol, 17.7% yield) and (S)-2-((1-(benzylamino)-5,6,7,8-tetrahydroisoquinolin-8-yl)amino)ethan-1-ol (1.50 g, 4.89 mmol, 21.3% yield) as a yellow oil. LC-MS: (ESI, m/z): 298.2 [M+H]⁺

Step 4: 5-(2-(((R)-1-(benzylamino)-5,6,7,8-tetrahydroisoquinolin-8-yl)amino)ethoxy)-7-(6-(bis(4-methoxybenzyl)amino)-4-methyl-3-(trifluoromethyl)pyridin-2-yl)-2,6-dichloro-8-fluoroquinazolin-4(3H)-one

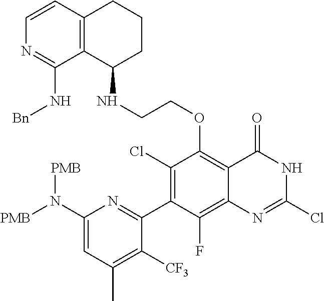

Similar to as described in General Procedure A. Under nitrogen, to a solution of (R)-2-((1-(benzylamino)-5,6,7,8-tetrahydroisoquinolin-8-yl)amino)ethan-1-ol (1.07 g, 3.61 mmol) in dimethyl sulfoxide (20 mL) was added sodium bis(trimethylsilyl)amide (9.02 mL, 9.02 mmol, 1 M in tetrahydrofuran) at 0° C. and stirred at 25° C. for 1 hour. Under nitrogen, the reaction mixture was added to a solution of 7-(6-(bis(4-methoxybenzyl)amino)-4-methyl-3-(trifluoromethyl)pyridin-2-yl)-2,6-dichloro-5,8-difluoroquinazolin-4(3H)-one (2.00 g, 3.01 mmol) in dimethyl sulfoxide (20 mL) at 25° C. and stirred at 25° C. for 1.5 hours. After completion, the reaction was quenched with ammonium chloride saturated solution, diluted with water, extracted with ethyl acetate, washed with water and the organic layer was combined. The organic layer was dried over anhydrous sodium sulfate and concentrated under vacuum to afford 5-(2-(((R)-1-(benzylamino)-5,6,7,8-tetrahydroisoquinolin-8-yl)amino)ethoxy)-7-(6-(bis(4-methoxybenzyl)amino)-4-methyl-3-(trifluoromethyl)pyridin-2-yl)-2,6-dichloro-8-fluoroquinazolin-4(3H)-one (3.02 g, 2.04 mmol, 67.8% yield) as a yellow solid. LC-MS: (ESI, m/z): 942.3 [M+H]⁺

Step 5: (8R)—N-benzyl-8-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)-5,6,7,8-tetrahydroisoquinolin-1-amine

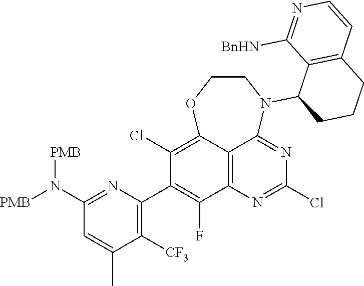

Similar to as described in General Procedure B. A solution of 5-(2-(((R)-1-(benzylamino)-5,6,7,8-tetrahydroisoquinolin-8-yl)amino)ethoxy)-7-(6-(bis(4-methoxybenzyl)amino)-4-methyl-3-(trifluoromethyl)pyridin-2-yl)-2,6-dichloro-8-fluoroquinazolin-4(3H)-one (2.90 g, 1.97 mmol), bis(2-oxo-3-oxazolidinyl)phosphinic chloride (601.3 mg, 2.36 mmol) and N,N-diisopropylethylamine (763.2 mg, 5.91 mmol) in chloroform (40 mL) was stirred at 70° C. for 1.5 hours. After completion, the reaction mixture was diluted with dichloromethane, washed with water and the organic layer was combined. The organic layer was dried over anhydrous sodium sulfate and concentrated under vacuum. The residue was purified by flash chromatography on silica gel eluting with petroleum ether/ethyl acetate (7:3) to afford (8R)—N-benzyl-8-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)-5,6,7,8-tetrahydroisoquinolin-1-amine (1.10 g, 1.15 mmol, 58.6% yield) as a yellow solid. LC-MS: (ESI, m/z): 924.3 [M+H]⁺

Step 6: (8R)—N-benzyl-8-(9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine

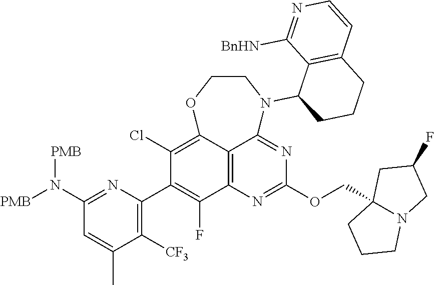

To a solution of ((2R,7aS)-2-fluorotetrahydro-1H-pyrrolizin-7a(5H)-yl)methanol (337.4 mg, 2.12 mmol) in tetrahydrofuran (15 mL) was added sodium hydride (169.5 mg, 4.24 mmol, 60% dispersion in mineral oil) at 0° C. and stirred at 25° C. for 30 min. Then (8R)—N-benzyl-8-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)-5,6,7,8-tetrahydroisoquinolin-1-amine (980.0 mg, 1.06 mmol) was added at 25° C. and stirred at 40° C. for 1.5 hours. After completion, the reaction was quenched with ammonium chloride saturated solution, concentrated under vacuum, diluted with dichloromethane, washed with water, dried over anhydrous sodium sulfate and concentrated under vacuum. The residue was purified by flash chromatography on silica gel eluting with petroleum ether/ethyl acetate (60:40) to afford (8R)—N-benzyl-8-(9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine (550.6 mg, 0.50 mmol, 47.1% yield). LC-MS: (ESI, m/z): 1047.4 [M+H]⁺

Step 7: (R)-8-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine & (R)-8-((S)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine

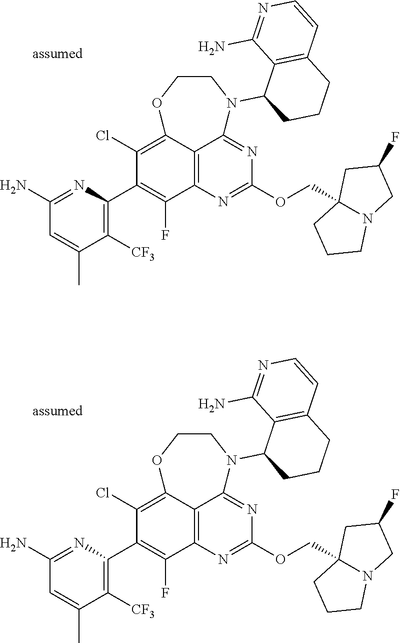

A solution of (8R)—N-benzyl-8-(9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine (530.0 mg, 0.48 mmol) in trifluoroacetic acid (10 mL) and trifluoromethanesulfonic acid (1 mL) was stirred for 5 hours at 25° C. After completion, the reaction mixture was adjusted to pH >7 with sodium bicarbonate saturated solution, extracted with dichloromethane, and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep C18 OBD Column, 30*100 mm, 5 μm; Mobile Phase A: water (10 mmol/L NH₄HCO₃), Mobile Phase B: acetonitrile; Flow rate: 60 mL/min; Gradient: 20% B to 50% B in 20 min, 50% B; Wave Length: 254/220 nm; RT1(min): 18.6 to afford (R)-8-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine (75.4 mg, 0.10 mmol, 21.1% yield) and (R)-8-((S)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)-5,6,7,8-tetrahydroisoquinolin-1-amine (44.2 mg, 0.06 mmol, 12.4% yield). LC-MS: (ESI, m/z): 717.3 [M+H]⁺

Example 65a: ¹H NMR (300 MHz, DMSO-d₆, ppm) δ 7.82 (d, J=5.1 Hz, 1H), 6.81 (s, 2H), 6.47 (d, J=5.4 Hz, 2H), 6.02 (t, J=5.7 Hz, 1H), 5.56-4.99 (m, 3H), 4.43-4.37 (m, 2H), 4.15 (d, J=10.2 Hz, 1H), 4.03 (d, J=10.5 Hz, 1H), 3.64-3.57 (m, 1H), 3.40-3.34 (m, 1H), 3.16-3.04 (m, 2H), 3.00 (s, 1H), 2.83-2.75 (m, 2H), 2.66-2.58 (m, 1H), 2.36 (d, J=1.2 Hz, 3H), 2.27-2.16 (m, 2H), 2.14-1.90 (m, 3H), 1.84-1.59 (m, 5H). LC-MS: (ESI, m/z): 717.3 [M+H]⁺

Example 65b: ¹H NMR (300 MHz, DMSO-d6, ppm) δ 7.82 (d, J=5.1 Hz, 1H), 6.81 (s, 2H), 6.47 (d, J=4.8 Hz, 2H), 6.05 (t, J=5.4 Hz, 1H), 5.36 (s, 2H), 5.19 (s, 1H), 4.52-4.37 (m, 2H), 4.13-4.05 (m, 2H), 3.74-3.60 (m, 1H), 3.38-3.31 (m, 1H), 3.16-3.03 (m, 2H), 3.00 (s, 1H), 2.83-2.75 (m, 2H), 2.67-2.60 (m, 1H), 2.36-2.32 (m, 3H), 2.21-2.09 (m, 2H), 2.08-1.96 (m, 3H), 1.93-1.59 (m, 5H). LC-MS: (ESI, m/z): 717.3 [M+H]⁺

Example 66a & 66b: 6-((R)-4-((R)-1-(2-aminopyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((R)-1-(2-aminopyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-aminium

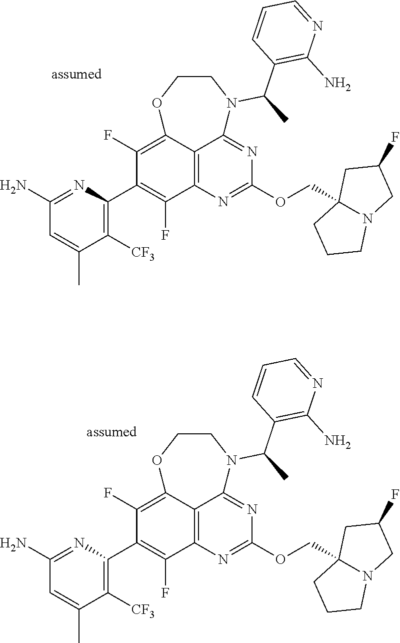

### Synthetic Route

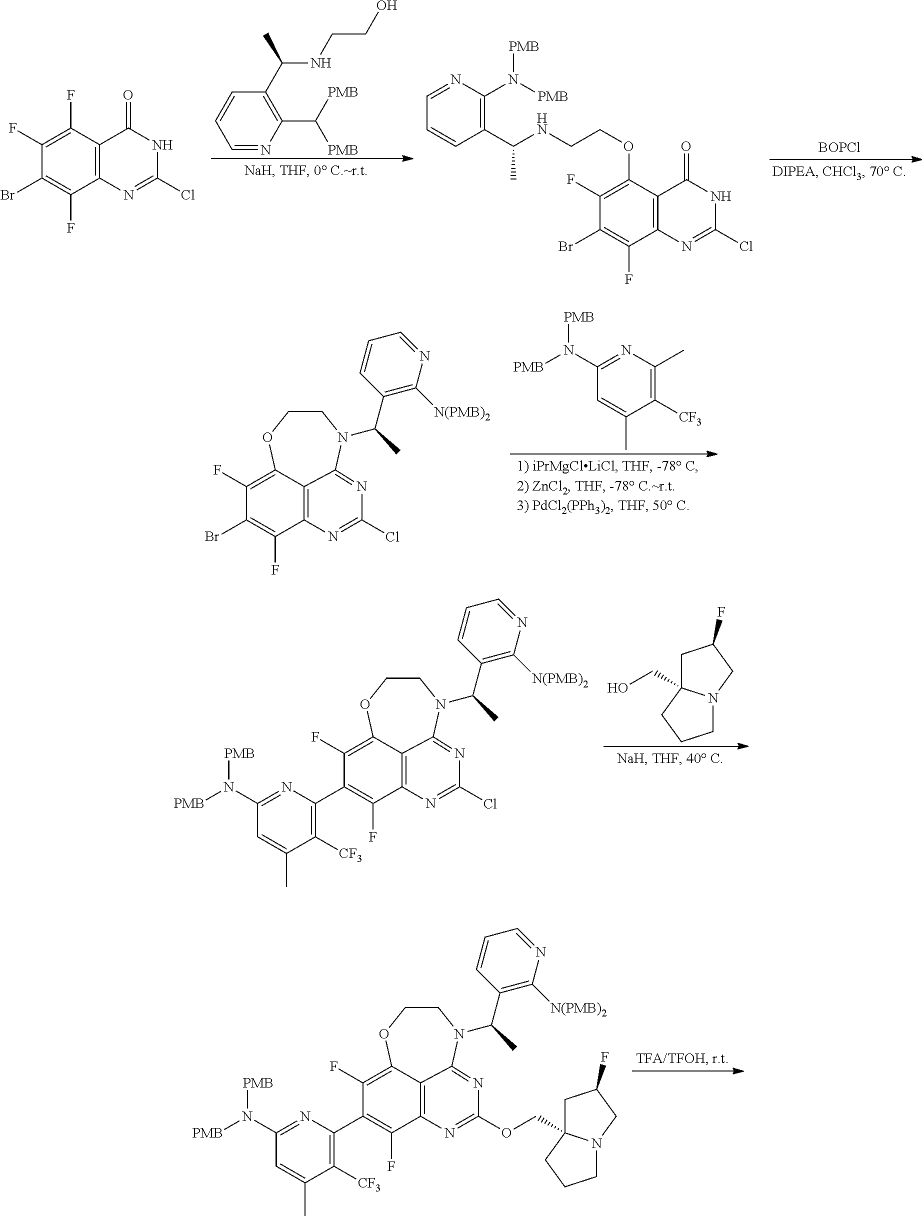

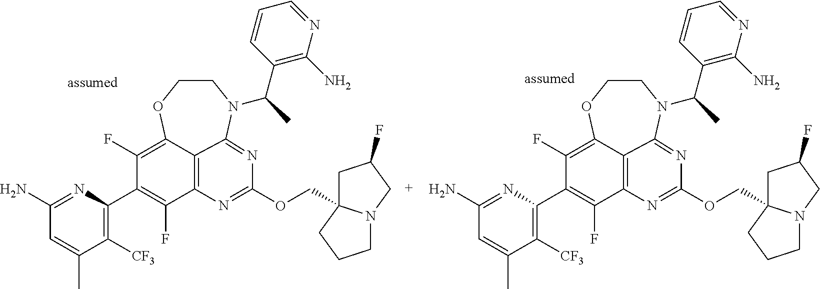

Step 1: (R)-5-(2-((1-(2-(bis(4-methoxybenzyl)amino)pyridin-3-yl)ethyl)amino)ethoxy)-7-bromo-2-chloro-6,8-difluoroquinazolin-4(3H)-one

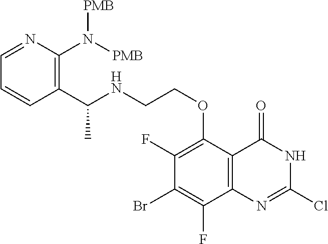

To a solution of (R)-2-((1-(2-(bis(4-methoxybenzyl)amino)pyridin-3-yl)ethyl)amino)ethan-1-ol (3.63 g, 8.61 mmol) in tetrahydrofuran (40 mL) was added sodium hydride (1.53 g, 38.28 mmol, 60% dispersion in mineral oil) at 0° C. and stirred at 0° C. for 30 min. Then the reaction mixture was added to a solution of 7-bromo-2-chloro-5,6,8-trifluoroquinazolin-4(3h)-one (3.00 g, 9.57 mmol) in tetrahydrofuran (10 mL) at 0° C. and stirred at 25° C. for 5 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with petroleum ether/ethyl acetate (60:40) to afford (R)-5-(2-((1-(2-(bis(4-methoxybenzyl)amino)pyridin-3-yl)ethyl)amino)ethoxy)-7-bromo-2-chloro-6,8-difluoroquinazolin-4(3H)-one (8.40 g, 4.93 mmol, 51.6% yield) as a yellow solid. LC-MS: (ESI, m/z): 714.1 [M+H]⁺

Step 2: (R)-3-(1-(9-bromo-2-chloro-8,10-difluoro-5,6-dihydro-4H-[1,4]oxazepino[5,6,7-de]quinazolin-4-yl)ethyl)-N,N-bis(4-methoxybenzyl)pyridin-2-amine

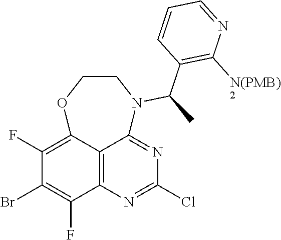

A solution of (R)-5-(2-((1-(2-(bis(4-methoxybenzyl)amino)pyridin-3-yl)ethyl)amino)ethoxy)-7-bromo-2-chloro-6,8-difluoroquinazolin-4(3H)-one (8.40 g, 4.93 mmol), bis(2-oxo-3-oxazolidinyl)phosphinic chloride (1.51 g, 5.92 mmol) and N,N-diisopropylethylamine (1.91 g, 14.8 mmol) in chloroform (25 mL) was stirred at 70° C. for 1.5 hours. After completion, the reaction mixture was diluted with dichloromethane, washed with water and the organic layer was combined. The organic layer was dried over anhydrous sodium sulfate and concentrated under vacuum. The residue was purified by flash chromatography on silica gel eluting with petroleum ether/ethyl acetate (7:3) to afford (R)-3-(1-(9-bromo-2-chloro-8,10-difluoro-5,6-dihydro-4H-[1,4]oxazepino[5,6,7-de]quinazolin-4-yl)ethyl)-N,N-bis(4-methoxybenzyl)pyridin-2-amine (1.50 g, 1.96 mmol, 39.7% yield) as a yellow solid. LC-MS: (ESI, m/z): 696.1 [M+H]⁺

Step 3: 6-(4-((R)-1-(2-(bis(4-methoxybenzyl)amino)pyridin-3-yl)ethyl)-2-chloro-8,10-difluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

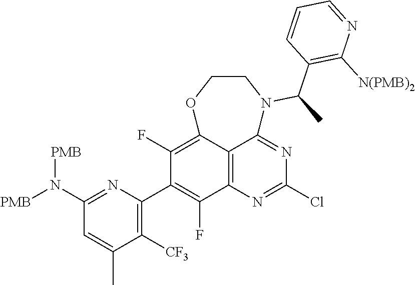

Under nitrogen, to a solution of (R)-3-(1-(9-bromo-2-chloro-8,10-difluoro-5,6-dihydro-4H-[1,4]oxazepino[5,6,7-de]quinazolin-4-yl)ethyl)-N,N-bis(4-methoxybenzyl)pyridin-2-amine (600.0 mg, 0.86 mmol) in tetrahydrofuran (5 mL) was added isopropylmagnesium chloride-lithium chloride complex (0.79 mL, 1.03 mmol, 1.3 M in tetrahydrofuran) at −78° C. and the reaction was stirred at −78° C. for 20 min. Then zinc chloride (1.29 mL, 2.58 mmol, 2 M in 2-methyltetrahydrofuran) was added at −78° C. The reaction was stirred at −78° C. for 5 min and stirred at 25° C. for 20 min. The mixture was transferred into a mixture of bis(triphenylphosphine)palladium(II) chloride (120.9 mg, 0.17 mmol) and 6-bromo-N,N-bis(4-methoxybenzyl)-4-methyl-5-(trifluoromethyl)pyridin-2-amine (469.1 mg, 0.95 mmol) in tetrahydrofuran (5 mL) and stirred at 50° C. for 1 hour. After completion, the reaction mixture was diluted with ethyl acetate, washed with water and the organic layer was combined. The organic layer was dried over anhydrous sodium sulfate and concentrated under vacuum. The residue was purified by flash chromatography on silica gel eluting with petroleum ether/ethyl acetate (7:3) to afford 6-(4-((R)-1-(2-(bis(4-methoxybenzyl)amino)pyridin-3-yl)ethyl)-2-chloro-8,10-difluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (410.4 mg, 0.31 mmol, 36% yield) as a white solid. LC-MS: (ESI, m/z): 1032.4 [M+H]⁺

Step 4: 6-(4-((R)-1-(2-(bis(4-methoxybenzyl)amino)pyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

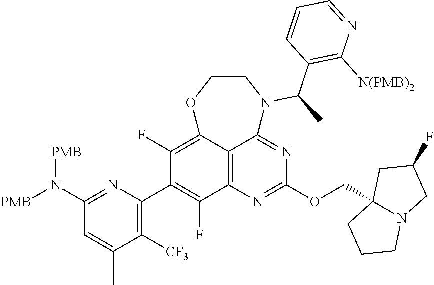

To a solution of ((2R,7aS)-2-fluorotetrahydro-1H-pyrrolizin-7a(5H)-yl)methanol (91.0 mg, 0.57 mmol) in tetrahydrofuran (5 mL) was added sodium hydride (57.2 mg, 1.43 mmol, 60% dispersion in mineral oil) at 0° C. and stirred at 25° C. for 30 min. Then 6-(4-((R)-1-(2-(bis(4-methoxybenzyl)amino)pyridin-3-yl)ethyl)-2-chloro-8,10-difluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (410.0 mg, 0.31 mmol) was added at 25° C. and stirred at 40° C. for 5 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with dichloromethane/methanol (93:7) to afford 6-(4-((R)-1-(2-(bis(4-methoxybenzyl)amino)pyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (180.3 mg, 0.15 mmol, 52.9% yield) as a white solid. LC-MS: (ESI, m/z): 1155.5 [M+H]⁺

Step 5: 6-((R)-4-((R)-1-(2-aminopyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((R)-1-(2-aminopyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-aminium

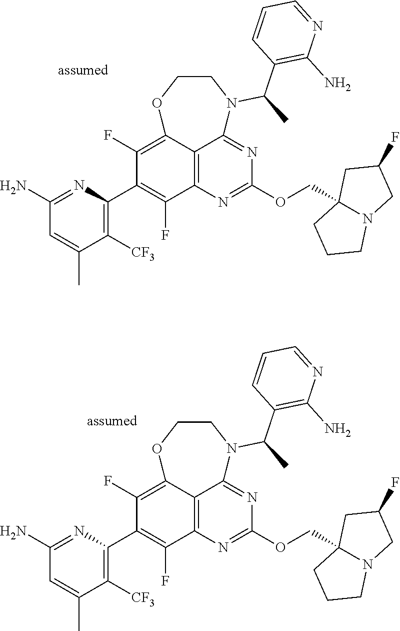

A solution of 6-(4-((R)-1-(2-(bis(4-methoxybenzyl)amino)pyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (76.0 mg, 0.06 mmol) in trifluoroacetic acid (3 mL) and trifluoromethanesulfonic acid (0.3 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 36% B to 61% B in 9 min, 61% B; Wave Length: 254/220 nm; RT1(min): 8.9 to afford 6-((R)-4-((R)-1-(2-aminopyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (21.4 mg, 0.03 mmol, 54.6% yield) and 6-((S)-4-((R)-1-(2-aminopyridin-3-yl)ethyl)-8,10-difluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-aminium (21.4 mg, 0.03 mmol, 54.6% yield). LC-MS: (ESI, m/z): 675.2 [M+H]⁺

Example 66a: ¹H NMR (300 MHz, DMSO-d₆, ppm) δ 7.96 (dd, J=4.8, 1.5 Hz, 1H), 7.63 (dd, J=7.8, 1.5 Hz, 1H), 6.86 (s, 2H), 6.66 (q, J=4.8 Hz, 1H), 6.49 (s, 1H), 6.31 (q, J=6.6 Hz, 1H), 5.73 (s, 2H), 5.28 (d, J=54.3 Hz, 1H), 4.48-4.18 (m, 2H), 4.15-3.98 (m, 2H), 3.73-3.57 (m, 1H), 3.46-3.41 (m, 1H), 3.17-3.03 (m, 2H), 3.00 (s, 1H), 2.91-2.74 (m, 1H), 2.36 (d, J=1.8 Hz, 3H), 2.28-2.12 (m, 1H), 2.11-1.94 (m, 2H), 1.94-1.66 (m, 3H), 1.56 (d, J=6.6 Hz, 3H). LC-MS: (ESI, m/z): 675.2 [M+H]⁺

Example 66b: ¹H NMR (300 MHz, DMSO-d₆, ppm) δ 7.96 (dd, J=5.1, 1.8 Hz, 1H), 7.64 (dd, J=7.5, 1.8 Hz, 1H), 6.86 (s, 2H), 6.65 (q, J=5.1 Hz, 1H), 6.49 (s, 1H), 6.32 (q, J=6.9 Hz, 1H), 5.82 (s, 2H), 5.28 (d, J=53.7 Hz, 1H), 4.54-4.38 (m, 1H), 4.33-4.19 (m, 1H), 4.07 (s, 2H), 3.79-3.63 (m, 1H), 3.47-3.37 (m, 1H), 3.18-3.03 (m, 2H), 3.00 (s, 1H), 2.92-2.74 (m, 1H), 2.37 (d, J=1.5 Hz, 3H), 2.23-2.12 (m, 1H), 2.11-1.93 (m, 2H), 1.92-1.70 (m, 3H), 1.57 (d, J=6.6 Hz, 3H). LC-MS: (ESI, m/z): 675.2 [M+H]⁺

Example 67: 6-((R)-4-((R)-1-(2-aminopyridin-3-yl)ethyl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-9-yl)-4-methyl-5-(trifluoromethyl)pyridin-2-amine

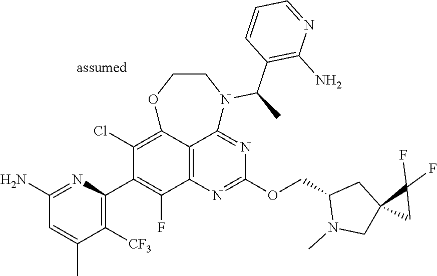

### Synthetic Route

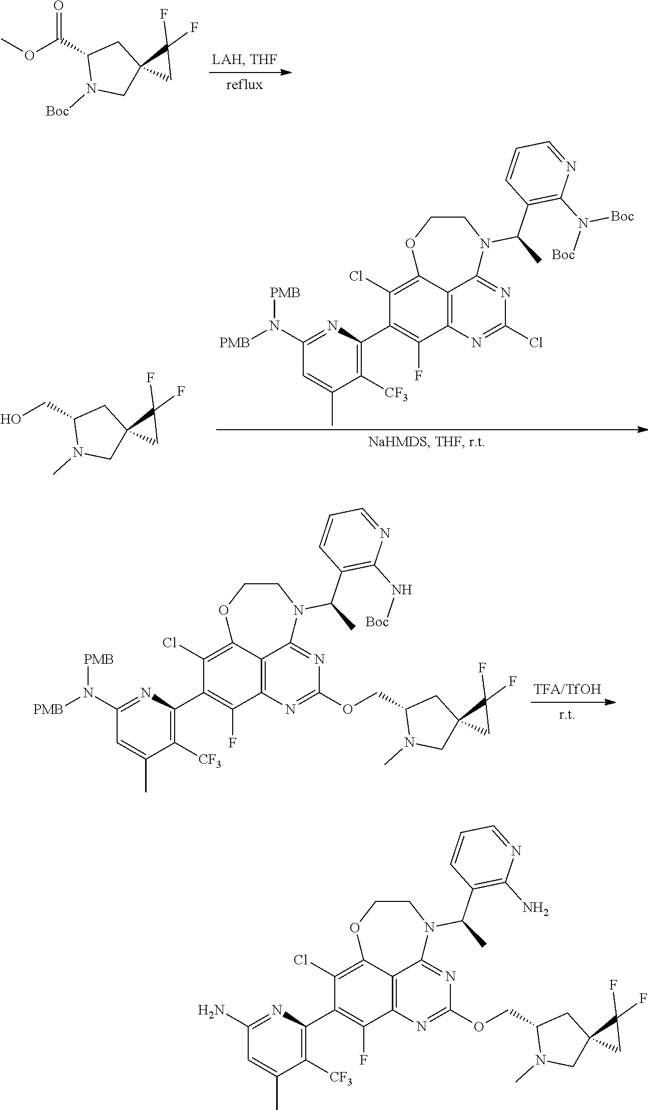

Step 1: ((3S,6S)-1,1-difluoro-5-methyl-5-azaspiro[2.4]heptan-6-yl)methanol

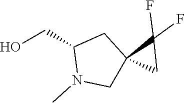

Lithiumaluminumhydride (97.7 mg, 2.57 mmol) was added to a solution of 5-(tert-butyl) 6-methyl (3S,6S)-1,1-difluoro-5-azaspiro[2.4]heptane-5,6-dicarboxylate (250.0 mg, 0.86 mmol) in tetrahydrofuran (5 mL) at 0° C. and stirred at 70° C. for 1 hour. After completion, the reaction was quenched by sodium sulfate decahydrate. After stirring at 25° C. for 20 min, the mixture was filtered. The filtrate was dried over sodium sulfate and concentrated under reduced pressure to afford ((3S,6S)-1,1-difluoro-5-methyl-5-azaspiro[2.4]heptan-6-yl)methanol (158.2 mg, 0.76 mmol, 88.3% yield) as a light yellow oil. LC-MS: (ESI, m/z): 178.1 [M+H]⁺

Step 2: tert-butyl (3-((R)-1-((R)-9-(6-(bis(4-methoxybenzyl)amino)-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyridin-2-yl)carbamate

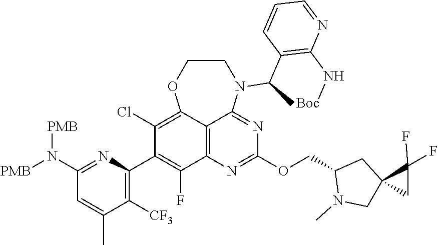

Under nitrogen, to a solution of ((3S,6S)-1,1-difluoro-5-methyl-5-azaspiro[2.4]heptan-6-yl)methanol (41.3 mg, 0.20 mmol) in tetrahydrofuran (2 mL) was added sodium bis(trimethylsilyl)amide (0.24 mL, 0.24 mmol, 1 M in tetrahydrofuran) at 0° C. and stirred at 25° C. for 15 min. Under nitrogen, the reaction mixture was added to a solution of tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (desired atropisomer) (80.0 mg, 0.08 mmol) in tetrahydrofuran (2 mL) at 25° C. and stirred at 25° C. for 2 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with petroleum ether/ethyl acetate (1:9) to afford tert-butyl (3-((R)-1-((R)-9-(6-(bis(4-methoxybenzyl)amino)-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyridin-2-yl)carbamate (70.3 mg, 0.06 mmol, 74.9% yield) as a white solid. LC-MS: (ESI, m/z): 1049.4 [M+H]⁺

Step 3: 6-((R)-4-((R)-1-(2-aminopyridin-3-yl)ethyl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-9-yl)-4-methyl-5-(trifluoromethyl)pyridin-2-amine

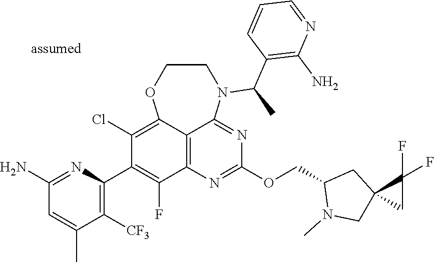

A solution of tert-butyl (3-((R)-1-((R)-9-(6-(bis(4-methoxybenzyl)amino)-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyridin-2-yl)carbamate (70.0 mg, 0.05 mmol) in trifluoroacetic acid (3 mL) and trifluoromethanesulfonic acid (0.3 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 58% B to 63% B in 9 min, 63% B: Wave Length: 254/220 nm; RT1(min): 8.01 to afford 6-((R)-4-((R)-1-(2-aminopyridin-3-yl)ethyl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-9-yl)-4-methyl-5-(trifluoromethyl)pyridin-2-amine (12.6 mg, 0.02 mmol, 32.6% yield). LC-MS: (ESI, m/z): 709.1 [M+H]⁺

Example 67: ¹H NMR (300 MHz, DMSO-d₆, ppm) δ 7.97 (dd, J=4.8, 1.5 Hz, 1H), 7.63 (dd, J=7.8, 1.5 Hz, 1H), 6.82 (s, 2H), 6.67 (q, J=4.9 Hz, 1H), 6.48 (s, 1H), 6.25 (q, J=6.9 Hz, 1H), 5.69 (s, 2H), 4.52-4.21 (m, 4H), 3.63-3.58 (m, 1H), 3.43-3.35 (m, 1H), 2.92-2.76 (m, 2H), 2.57 (d, J=9.9 Hz, 1H), 2.37 (s, 6H), 2.27-2.16 (m, 1H), 1.85-1.67 (m, 1H), 1.60-1.47 (m, 4H), 1.49-1.32 (m, 1H). LC-MS: (ESI, m/z): 709.1 [M+H]⁺

Example 68: 6-((R)-4-((R)-1-(2-aminopyridin-3-yl)ethyl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-9-yl)-4-methyl-5-(trifluoromethyl)pyridin-2-amine

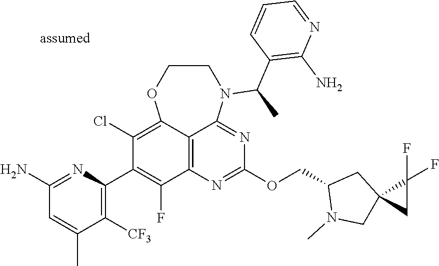

### Synthetic Route

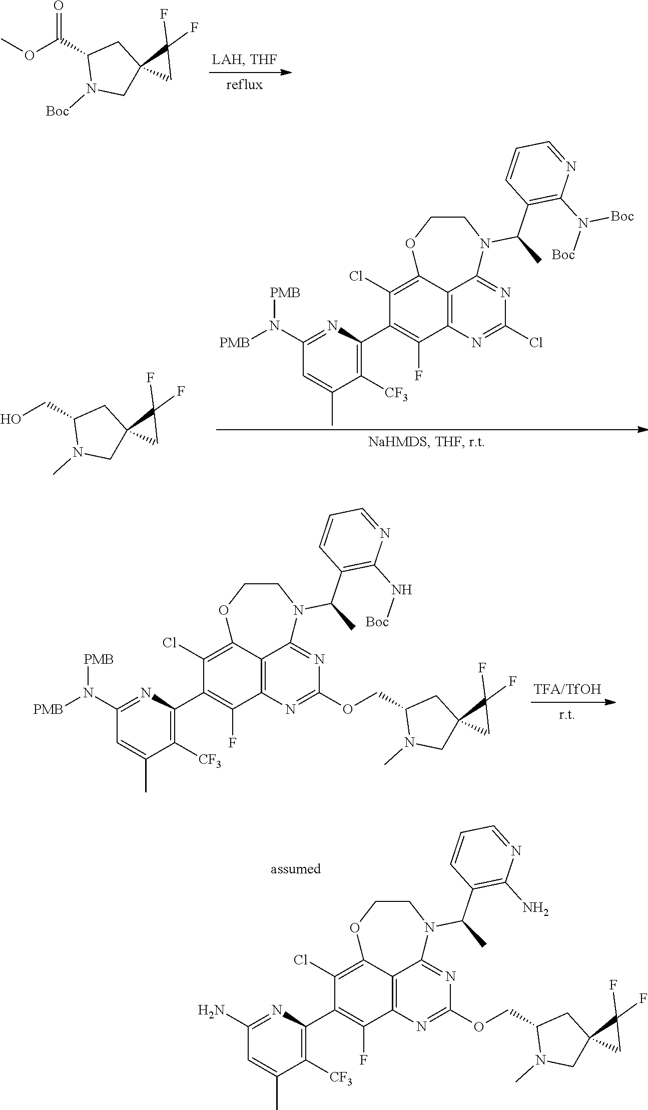

Step 1: ((3R,6S)-1,1-difluoro-5-methyl-5-azaspiro[2.4]heptan-6-yl)methanol

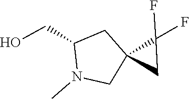

Lithiumaluminumhydride (97.7 mg, 2.57 mmol) was added to a solution of 5-(tert-butyl) 6-methyl (3R,6S)-1,1-difluoro-5-azaspiro[2.4]heptane-5,6-dicarboxylate (250.0 mg, 0.86 mmol) in tetrahydrofuran (4 mL) at 0° C. and stirred at 70° C. for 1 hour. After completion, the reaction was quenched by sodium sulfate decahydrate. After stirring at 25° C. for 20 min, the mixture was filtered. The filtrate was dried over sodium sulfate and concentrated under reduced pressure to afford ((3R,6S)-1,1-difluoro-5-methyl-5-azaspiro[2.4]heptan-6-yl)methanol (165.2 mg, 0.85 mmol, 98.7% yield) as a light yellow oil. LC-MS: (ESI, m/z): 178.1 [M+H]⁺

Step 2: tert-butyl (3-((R)-1-((R)-9-(6-(bis(4-methoxybenzyl)amino)-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyridin-2-yl)carbamate

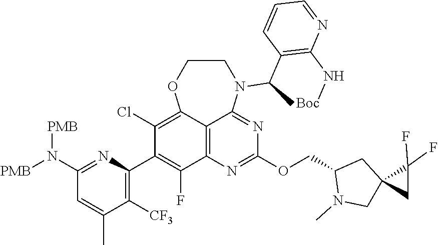

Under nitrogen, to a solution of ((3R,6S)-1,1-difluoro-5-methyl-5-azaspiro[2.4]heptan-6-yl)methanol (72.4 mg, 0.37 mmol) in tetrahydrofuran (2.5 mL) was added sodium bis(trimethylsilyl)amide (0.45 mL, 0.45 mmol, 1 M in tetrahydrofuran) at 0° C. and stirred at 25° C. for 1 hour. Under nitrogen, the reaction mixture was added to a solution of tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (desired atropisomer) (150.0 mg, 0.15 mmol) in tetrahydrofuran (2.5 mL) at 25° C. and stirred at 25° C. for 5 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with dichloromethane/ethyl acetate (7:3) to afford tert-butyl (3-((R)-1-((R)-9-(6-(bis(4-methoxybenzyl)amino)-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyridin-2-yl)carbamate (76.4 mg, 0.06 mmol, 42.9% yield) as a white solid. LC-MS: (ESI, m/z): 1049.4 [M+H]⁺

Step 3: 6-((R)-4-((R)-1-(2-aminopyridin-3-yl)ethyl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-9-yl)-4-methyl-5-(trifluoromethyl)pyridin-2-amine

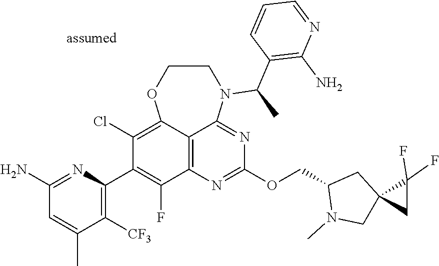

A solution of tert-butyl (3-((R)-1-((R)-9-(6-(bis(4-methoxybenzyl)amino)-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyridin-2-yl)carbamate (70.0 mg, 0.05 mmol) in trifluoroacetic acid (3 mL) and trifluoromethanesulfonic acid (0.3 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 36% B to 61% B in 9 min, 61% B; Wave Length: 254/220 nm; RT1(min): 8.6 to afford 6-((R)-4-((R)-1-(2-aminopyridin-3-yl)ethyl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-9-yl)-4-methyl-5-(trifluoromethyl)pyridin-2-amine (17.1 mg, 0.02 mmol, 44.5% yield). LC-MS: (ESI, m/z): 709.2 [M+H]⁺

Example 68: ¹H NMR (300 MHz, DMSO-d₆, ppm) δ 7.90 (dd, J=4.8, 1.5 Hz, 1H), 7.57 (dd, J=7.5, 1.2 Hz, 1H), 6.75 (s, 2H), 6.60 (q, J=4.9 Hz, 1H), 6.41 (s, 1H), 6.19 (q, J=6.6 Hz, 1H), 5.59 (s, 2H), 4.45-4.32 (m, 2H), 4.28-4.15 (m, 2H), 3.62-3.49 (m, 1H), 3.38-3.25 (m, 1H), 2.93-2.76 (m, 2H), 2.46-2.37 (m, 1H), 2.36-2.23 (m, 6H), 2.09-1.92 (m, 1H), 1.91-1.72 (m, 1H), 1.50 (d, J=6.6 Hz, 3H), 1.45-1.30 (m, 2H). LC-MS: (ESI, m/z): 709.2 [M+H]⁺

Example 69a & 69b & 69c & 69d: 6-((R)-4-((R)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((R)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((S)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((S)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

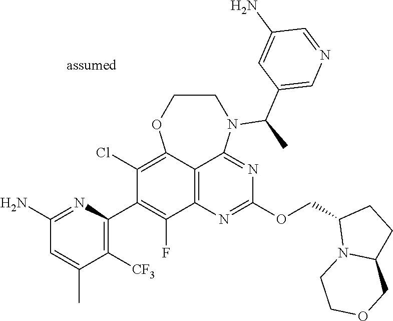

### Synthetic Route

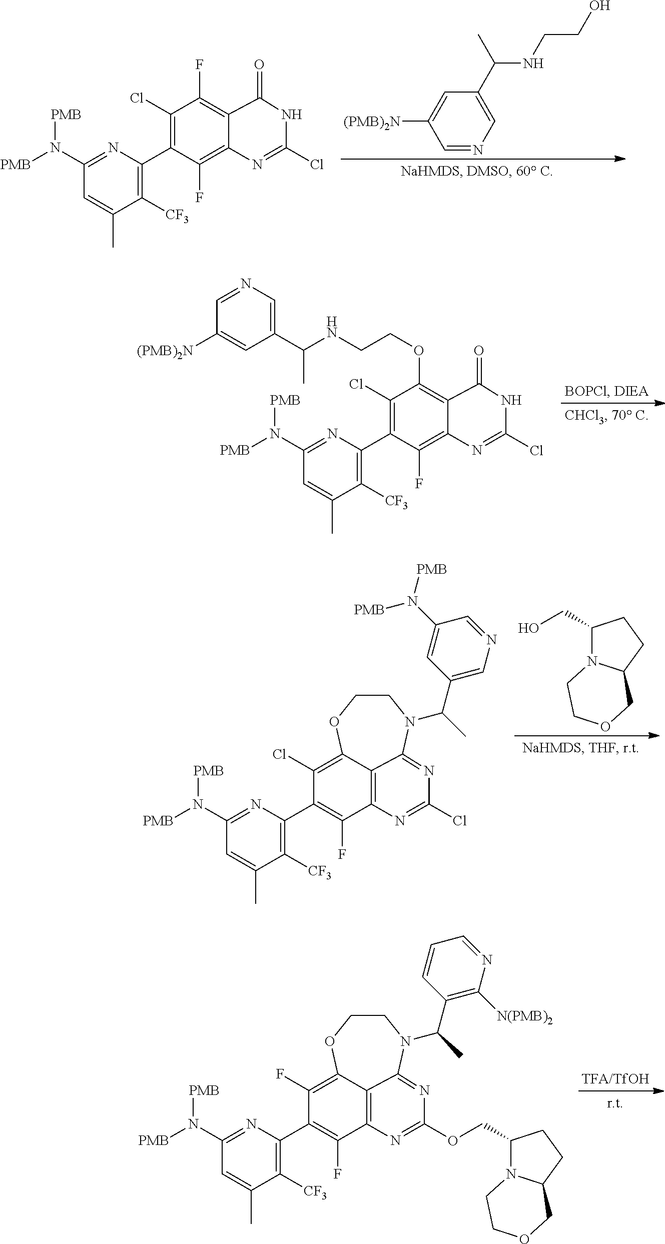

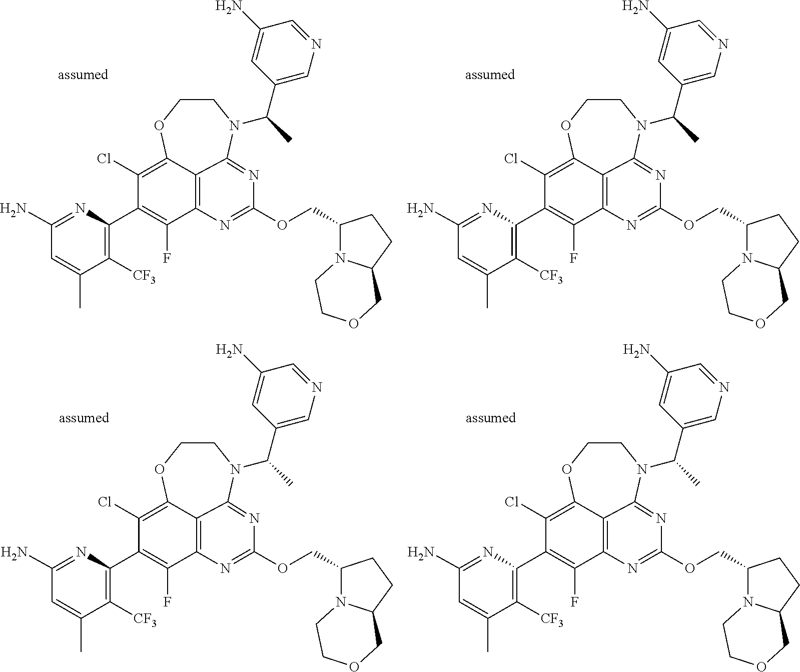

Step 1: 7-(6-(bis(4-methoxybenzyl)amino)-4-methyl-3-(trifluoromethyl)pyridin-2-yl)-5-(2-((1-(5-(bis(4-methoxybenzyl)amino)pyridin-3-yl)ethyl)amino)ethoxy)-2,6-dichloro-8-fluoroquinazolin-4(3H)-one

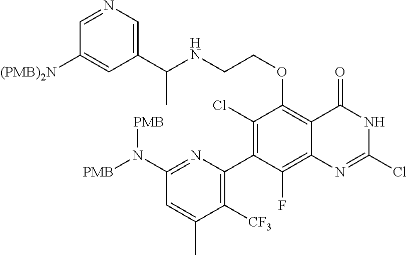

Similar to as described in General Procedure A. Under nitrogen, sodium bis(trimethylsilyl)amide (22.54 mL, 22.54 mmol, 1 M in tetrahydrofuran) was added to a solution of 2-((1-(5-(bis(4-methoxybenzyl)amino)pyridin-3-yl)ethyl)amino)ethan-1-ol (3.50 g, 8.30 mmol) in dimethyl sulfoxide (20 mL) and stirred for 20 min at 60° C. Then 7-(6-(bis(4-methoxybenzyl)amino)-4-methyl-3-(trifluoromethyl)pyridin-2-yl)-2,6-dichloro-5,8-difluoroquinazolin-4(3H)-one (5.00 g, 7.51 mmol) in dimethyl sulfoxide (20 mL) was added and stirred at 60° C. for 2 hours. After completion, the reaction was quenched with saturated ammonium chloride solution, diluted with water, extracted with ethyl acetate, washed with water and the organic layer was combined. The organic layer was dried over anhydrous sodium sulfate and concentrated under vacuum. The residue was purified by flash chromatography on silica gel eluting with dichloromethane/methanol (10:1) to afford 7-(6-(bis(4-methoxybenzyl)amino)-4-methyl-3-(trifluoromethyl)pyridin-2-yl)-5-(2-((1-(5-(bis(4-methoxybenzyl)amino)pyridin-3-yl)ethyl)amino)ethoxy)-2,6-dichloro-8-fluoroquinazolin-4(3H)-one (5.00 g, 4.69 mmol, 62.4% yield) as a yellow solid. LC-MS: (ESI, m/z): 1066.3 [M+H]⁺

Step 2: 6-(4-(1-(5-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

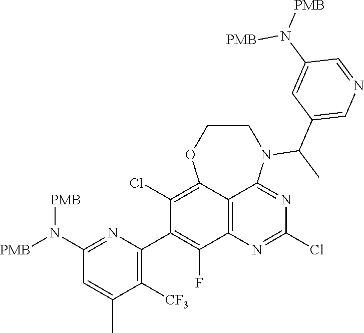

Similar to as described in General Procedure B. A solution of 7-(6-(bis(4-methoxybenzyl)amino)-4-methyl-3-(trifluoromethyl)pyridin-2-yl)-5-(2-((1-(5-(bis(4-methoxybenzyl)amino)pyridin-3-yl)ethyl)amino)ethoxy)-2,6-dichloro-8-fluoroquinazolin-4(3H)-one (1.20 g, 1.12 mmol), N,N-diisopropylethylamine (1.44 g, 11.16 mmol) and bis(2-oxo-3-oxazolidinyl)phosphinic chloride (360.0 mg, 1.41 mmol) in chloroform (10 mL) was stirred at 70° C. for 2 hours. After completion, the reaction mixture was diluted with dichloromethane, washed with water and the organic layer was combined. The organic layer was dried over anhydrous sodium sulfate and concentrated under vacuum. The residue was purified by flash chromatography on silica gel eluting with petroleum ether/ethyl acetate (1:1) to afford 6-(4-(1-(5-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (4.0 g, 3.81 mmol, 81.4% yield) as a yellow solid. LC-MS: (ESI, m/z): 1048.3 [M+H]⁺

Step 3: 6-(4-(1-(5-(bis(4-methoxybenzyl)amino)pyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

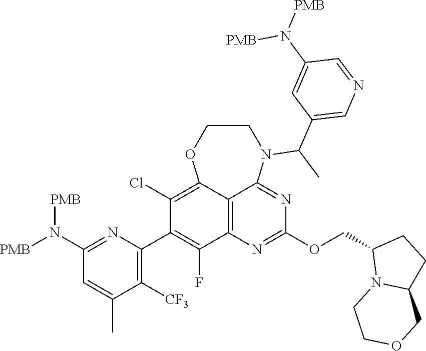

Under nitrogen, sodium bis(trimethylsilyl)amide (3.81 mL, 3.81 mmol, 1 M in tetrahydrofuran) was added to a solution of ((6S,8aS)-hexahydro-1H-pyrrolo[2,1-c][1,4]oxazin-6-yl)methanol (749.0 mg, 4.76 mmol) in tetrahydrofuran (10 mL) was stirred for 20 min at 25° C. Then 6-(4-(1-(5-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (1.00 g, 0.95 mmol) in tetrahydrofuran (10 mL) was added and stirred at 25° C. for 1 hour. After completion, the reaction was quenched with saturated ammonium chloride solution, diluted with ethyl acetate, washed with water and the organic layer was combined. The organic layer was dried over anhydrous sodium sulfate and concentrated under vacuum. The residue was purified by flash chromatography on silica gel eluting with dichloromethane/methanol (9:1) to afford 6-(4-(1-(5-(bis(4-methoxybenzyl)amino)pyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (600.0 mg, 0.51 mmol, 53.8% yield) as a yellow solid. LC-MS: (ESI, m/z): 1169.5 [M+H]⁺

Step 4: 6-((R)-4-((R)-1-(5-aminopyridin-3-yl)ethyl)-8-chloro-1 O-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((R)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((S)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((S)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6, 7-de]quinazolin-9-yl)-4-methyl-5-(trifluoromethyl)pyridin-2-amine

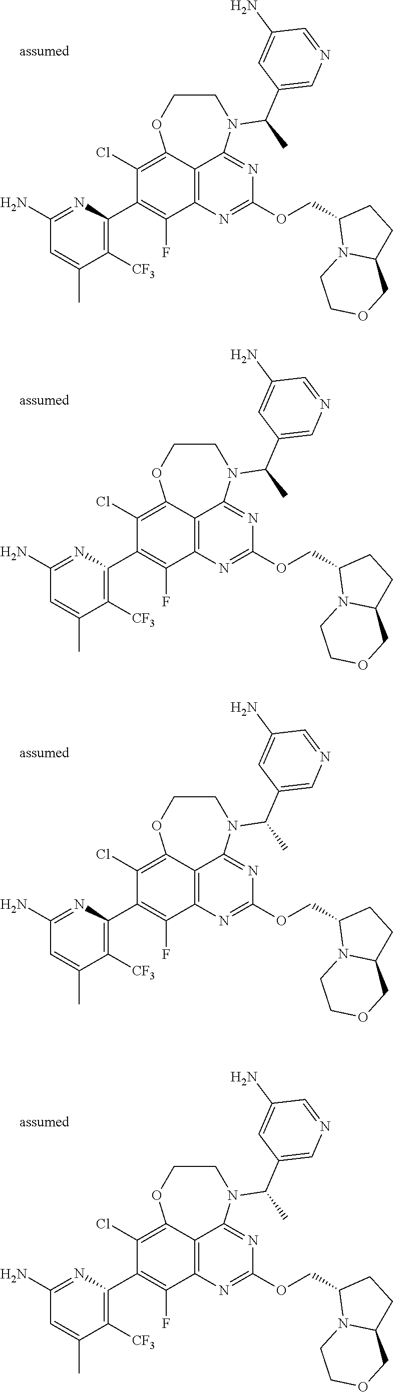

A solution of 6-(4-(I-(5-(bis(4-methoxybenzyl)amino)pyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (590.0 mg, 0.50 mmol) in trifluoroacetic acid (5 mL) and trifluoromethanesulfonic acid (0.5 mL) was stirred for 5 hours at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 35% B to 49% B in 10 min, 49% B; Wave Length: 2541220 nm; RT1(min): 8.9 to afford two diastereoisomers. The diastereoisomer (faster peak) was isolated by Prep-Chiral-HPLC with the following conditions: Column: CHIRAL ART Cellulose-SC, 2*25 cm, 5 μm; Mobile Phase A: Hex (0.5% 2M NH₃-MeOH)-HPLC, Mobile Phase B: EtOH-HPLC; Flow rate: 15 mL/min; Gradient: 50% B to 50% B in 23 min; Wave Length: 220/254 nm; RT1(min): 11.224; RT2(min): 18.727; Sample Solvent: EtOH-HPLC; Injection Volume: 0.8 mL to afford 6-((R)-4-((R)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (36.6 mg, 0.05 mmol, 10.1% yield) and 6-((S)-4-((R)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (34.2 mg, 0.05 mmol, 9.7% yield). The diastereoisomer (slower peak) was isolated by Prep-Chiral-HPLC with the following conditions: Column: CHIRAL ART Cellulose-SC, 2*25 cm, 5 μm; Mobile Phase A: Hex (0.5% 2M NH3-MeOH)-HPLC, Mobile Phase B: EtOH-HPLC; Flow rate: 20 mL/min; Gradient: 50% B to 50% B in 17 min; Wave Length: 220/254 nm; RT1(min): 8.377; RT2(min): 13.471; Sample Solvent: EtOH-HPLC; Injection Volume: 1 mL to afford 6-((R)-4-((S)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (28.6 mg, 0.04 mmol, 8.1% yield) and 6-((S)-4-((S)-1-(5-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (34.2 mg, 0.05 mmol, 9.6% yield). LC-MS: (ESI, m/z): 689.2 [M+H]⁺

Example 69a: ¹H NMR (300 MHz, DMSO-d₆, ppm) δ 7.88 (d, J=2.4 Hz, 1H), 7.82 (d, J=1.5 Hz, 1H), 6.90 (t, J=1.8 Hz, 1H), 6.82 (s, 2H), 6.49 (s, 1H), 6.40 (q, J=6.9 Hz, 1H), 5.31 (s, 2H), 4.60-4.44 (m, 1H), 4.43-4.29 (m, 2H), 4.15-3.98 (m, 1H), 3.75-3.63 (m, 1H), 3.63-3.56 (m, 1H), 3.54-3.48 (m, 2H), 3.48-3.38 (m, 2H), 3.21-3.11 (m, 1H), 3.04-2.90 (m, 1H), 2.89-2.78 (m, 2H), 2.37 (d, J=1.5 Hz, 3H), 2.18-1.95 (m, 1H), 1.84-1.69 (m, 1H), 1.61 (d, J=6.9 Hz, 4H), 1.42-1.22 (m, 1H). LC-MS: (ESI, m/z): 689.2 [M+H]⁺ CHIRALPAK IC-3, 4.6×50 mm, 3 um; detected at 254 nm; Hex (0.1% DEA):EtOH=50:50; Flow rate: 1 mL/min; Retention time: 2.426 min (faster peak)

Example 69b: ¹H NMR (300 MHz, DMSO-d₆, ppm) δ 7.88 (d, J=2.4 Hz, 1H), 7.82 (d, J=1.5 Hz, 1H), 6.90 (t, J=1.8 Hz, 1H), 6.82 (s, 2H), 6.49 (s, 1H), 6.40 (q, J=6.9 Hz, 1H), 5.31 (s, 2H), 4.58-4.46 (m, 1H), 4.43-4.39 (m, 1H), 4.37-4.28 (m, 1H), 4.18-4.08 (m, 1H), 3.79-3.63 (m, 1H), 3.63-3.55 (m, 1H), 3.55-3.45 (m, 3H), 3.45-3.37 (m, 1H), 3.19-3.06 (m, 1H), 3.02-2.91 (m, 1H), 2.90-2.79 (m, 2H), 2.37 (d, J=1.2 Hz, 3H), 2.07-1.96 (m, 1H), 1.83-1.67 (m, 1H), 1.61 (d, J=6.9 Hz, 3H), 1.55-1.42 (m, 1H), 1.39-1.22 (m, 1H). LC-MS: (ESI, m/z): 689.2 [M+H]⁺ CHIRALPAK IC-3, 4.6×50 mm, 3 um; detected at 254 nm; Hex (0.1% DEA):EtOH=50:50; Flow rate: 1 mL/min; Retention time: 4.440 min (slower peak)

Example 69c: ¹H NMR (300 MHz, DMSO-d₆, ppm) δ 7.88 (d, J=2.4 Hz, 1H), 7.80 (d, J=1.8 Hz, 1H), 6.87 (t, J=2.3 Hz, 1H), 6.81 (s, 2H), 6.49 (s, 1H), 6.40 (q, J=6.9 Hz, 1H), 5.31 (s, 2H), 4.68-4.50 (m, 1H), 4.46-4.40 (m, 1H), 4.39-4.32 (m, 1H), 4.18-4.01 (m, 1H), 3.84-3.66 (m, 1H), 3.66-3.51 (m, 1H), 3.51-3.37 (m, 4H), 3.22-3.03 (m, 1H), 3.01-2.90 (m, 1H), 2.90-2.78 (m, 2H), 2.37 (d, J=1.5 Hz, 3H), 2.07-1.92 (m, 1H), 1.81-1.67 (m, 1H), 1.60 (d, J=6.0 Hz, 3H), 1.53-1.38 (m, 1H), 1.36-1.21 (m, 1H). LC-MS: (ESI, m/z): 689.2 [M+H]⁺ CHIRALPAK IC-3, 4.6×50 mm, 3 um; detected at 254 nm; Hex (0.1% DEA):EtOH=50:50; Flow rate: 1 mL/min; Retention time: 2.188 min (faster peak)

Example 69d: ¹H NMR (300 MHz, DMSO-d6, ppm) δ 7.87 (d, J=2.4 Hz, 1H), 7.80 (d, J=1.8 Hz, 1H), 6.87 (t, J=2.4 Hz, 1H), 6.81 (s, 2H), 6.49 (s, 1H), 6.40 (q, J=6.8 Hz, 1H), 5.31 (s, 2H), 4.67-4.49 (m, 1H), 4.47-4.31 (m, 2H), 4.15-4.03 (m, 1H), 3.79-3.69 (m, 1H), 3.63-3.55 (m, 1H), 3.54-3.40 (m, 2H), 3.39-3.36 (m, 2H), 3.17-3.04 (m, 1H), 3.03-2.92 (m, 1H), 2.90-2.75 (m, 2H), 2.37 (d, J=1.5 Hz, 3H), 2.15-1.96 (m, 1H), 1.84-1.68 (m, 1H), 1.61 (d, J=6.9 Hz, 3H), 1.59-1.50 (m, 1H), 1.35-1.21 (m, 1H). LC-MS: (ESI, m/z): 689.1 [M+H]⁺ CHIRALPAK IC-3, 4.6×50 mm, 3 um; detected at 254 nm; Hex (0.1% DEA):EtOH=50:50; Flow rate: 1 mL/min: Retention time: 4.315 min (slower peak)

Example 70a & 70b & 70c & 70d: 6-((R)-4-((R)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((S)-4-((R)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((R)-4-((S)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((S)-4-((S)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

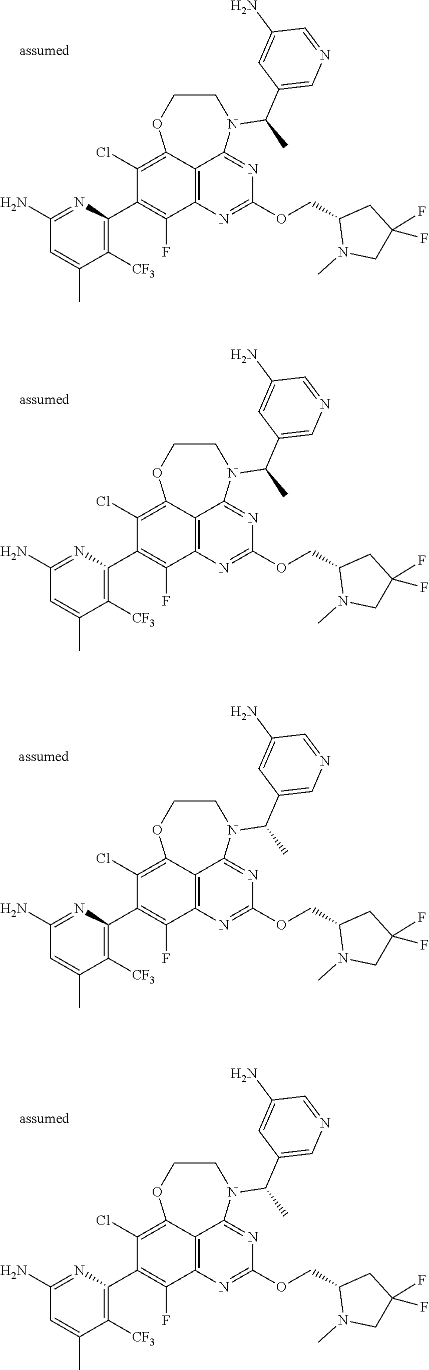

### Synthetic Route

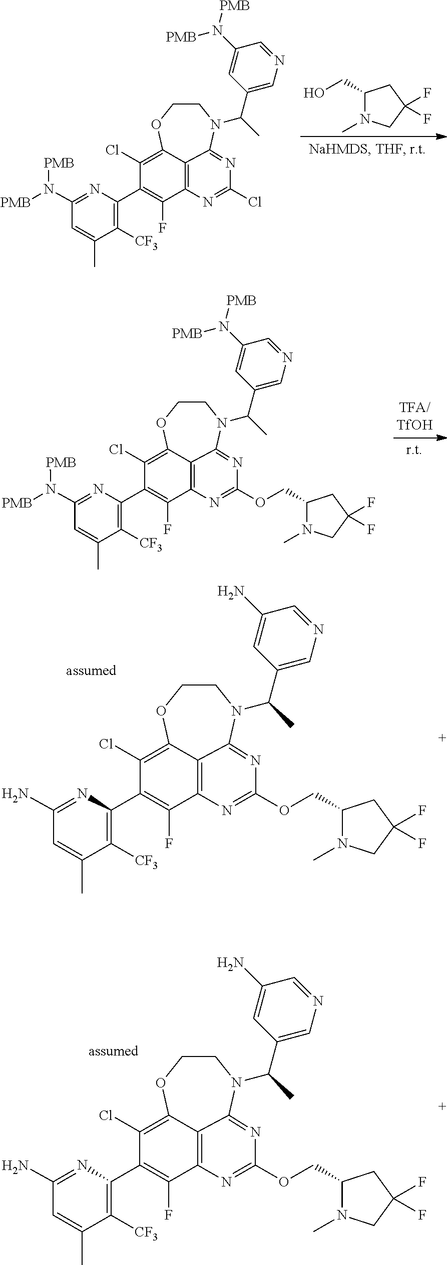

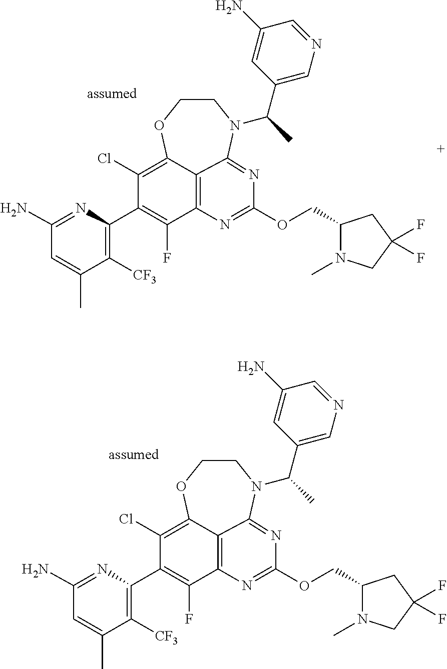

Step 1: 6-(4-(1-(5-(bis(4-methoxybenzyl)amino)pyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

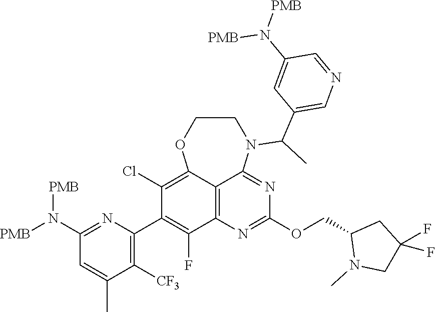

Under nitrogen, sodium bis(trimethylsilyl)amide (3.05 mL, 3.05 mmol, 1 M in tetrahydrofuran) was added to a solution of (S)-(4,4-difluoro-1-methylpyrrolidin-2-yl)methanol (580.0 mg, 3.84 mmol) in tetrahydrofuran (10 mL) was stirred for 20 min at 25° C. Then 6-(4-(1-(5-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (800.0 mg, 0.76 mmol) in tetrahydrofuran (10 mL) was added and stirred at 25° C. for 1 hour. After completion, the reaction was quenched with saturated ammonium chloride solution, diluted with ethyl acetate, washed with water and the organic layer was combined. The organic layer was dried over anhydrous sodium sulfate and concentrated under vacuum. The residue was purified by flash chromatography on silica gel eluting with dichloromethane/methanol (10:1) to afford 6-(4-(1-(5-(bis(4-methoxybenzyl)amino)pyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.3 mg, 0.34 mmol, 45.1% yield) as a yellow solid. LC-MS: (ESI, m/z): 1163.4 [M+H]⁺

Step 2: 6-((R)-4-((R)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((S)-4-((R)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((R)-4-((S)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((S)-4-((S)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

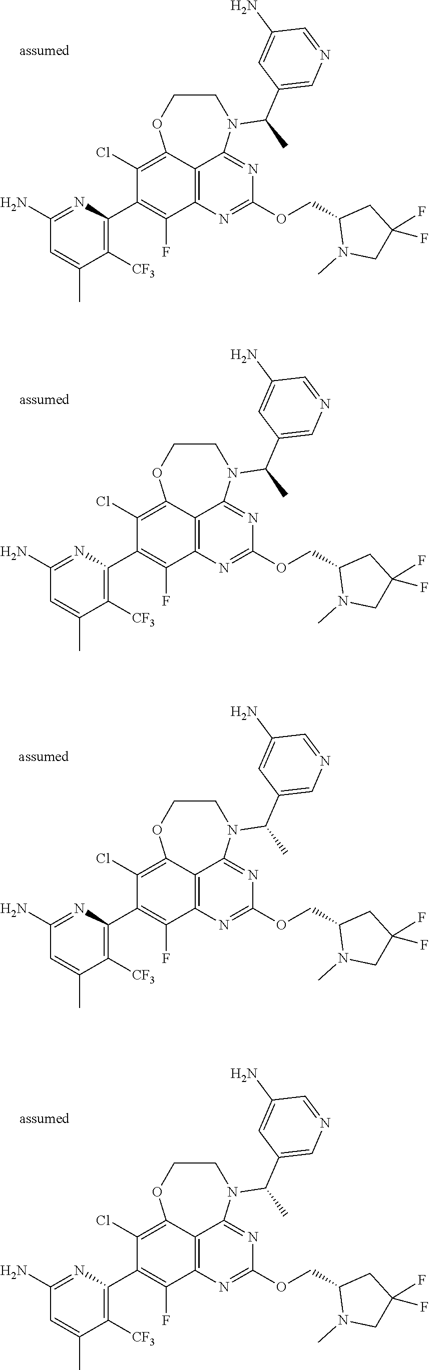

A solution of 6-(4-(1-(5-(bis(4-methoxybenzyl)amino)pyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (320.0 mg, 0.27 mmol) in trifluoroacetic acid (5 mL) and trifluoromethanesulfonic acid (0.5 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min: Gradient: 35% B to 49% B in 10 min, 49% B; Wave Length: 254/220 nm; RT1(min): 8.9 to afford two diastereoisomers. The diastereoisomer (faster peak) was isolated by Prep-Chiral-HPLC with the following conditions: Column: CHIRAL ART Cellulose-SC, 2*25 cm, 5 μm; Mobile Phase A: Hex:DCM=3:1 (0.5% 2M NH₃-MeOH)-HPLC, Mobile Phase B: EtOH-HPLC: Flow rate: 20 mL/min; Gradient: 30% B to 30% B in 9 min; Wave Length: 220/254 nm; RT1(min): 5.223; RT2(min): 7.249; Sample Solvent: EtOH-HPLC; Injection Volume: 0.6 mL to afford 6-((R)-4-((R)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine (25.0 mg, 0.03 mmol, 12.7% yield) and 6-((S)-4-((R)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine (23.0 mg, 0.03 mmol, 12.2% yield). The diastereoisomer (slower peak) was isolated by Prep-Chiral-HPLC with the following conditions: Column: CHIRAL ART Cellulose-SC, 2*25 cm, 5 μm; Mobile Phase A: Hex:DCM=3:1 (0.5% 2M NH₃-MeOH)-HPLC, Mobile Phase B: EtOH-HPLC; Flow rate: 20 mL/min; Gradient: 30% B to 30% B in 10 min; Wave Length: 220/254 nm; RT1(min): 5.393; RT2(min): 7.934; Sample Solvent: EtOH-HPLC: Injection Volume: 0.7 mL to afford 6-((R)-4-((S)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine (19.9 mg, 0.03 mmol, 10.3% yield) and 6-((S)-4-((S)-1-(5-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine (25.9 mg, 0.04 mmol, 13.7% yield). LC-MS: (ESI, m/z): 683.4 [M+H]⁺

Example 70a: 1H NMR (400 MHz, DMSO-d₆, ppm) δ 7.88 (d, J=2.4 Hz, 1H), 7.81 (d, J=1.6 Hz, 1H), 6.90 (t, J=2.5 Hz, 1H), 6.85 (s, 2H), 6.49 (s, 1H), 6.41 (q, J=7.0 Hz, 1H), 5.33 (s, 2H), 4.60-4.22 (m, 4H), 3.78-3.63 (m, 1H), 3.51-3.41 (m, 1H), 3.36-3.28 (m, 1H), 2.98-2.85 (m, 1H), 2.72-2.53 (m, 1H), 2.49-2.40 (m, 1H), 2.37 (d, J=6.8 Hz, 3H), 2.33 (s, 3H), 2.27-2.11 (m, 1H), 1.60 (d, J=6.8 Hz, 3H). LC-MS: (ESI, m/z): 683.4 [M+H]⁺ CHIRALPAK IC-3, 4.6×50 mm, 3 um; detected at 254 nm; (Hex:DCM=3:1) (0.1% DEA):EtOH=70:30; Flow rate: 1 mL/min; Retention time: 1.306 min (faster peak)

Example 70b: ¹H NMR (400 MHz, DMSO-d₆, ppm) δ 7.88 (d, J=2.4 Hz, 1H), 7.82 (d, J=1.2 Hz, 1H), 6.90 (t, J=2.5 Hz, 1H), 6.85 (s, 2H), 6.49 (s, 1H), 6.41 (q, J=7.0 Hz, 1H), 5.33 (s, 2H), 4.64-4.28 (m, 4H), 3.77-3.62 (m, 1H), 3.51-3.42 (m, 1H), 3.38-3.29 (m, 1H), 2.99-2.81 (m, 1H), 2.74-2.55 (m, 1H), 2.48-2.40 (m, 1H), 2.39-2.29 (m, 6H), 2.27-2.10 (m, 1H), 1.61 (d, J=7.2 Hz, 3H). LC-MS: (ESI, m/z): 683.3 [M+H]⁺ CHIRALPAK IC-3, 4.6×50 mm, 3 um; detected at 254 nm; (Hex:DCM=3:1) (0.1% DEA):EtOH=70:30; Flow rate: 1 mL/min; Retention time: 2.206 min (slower peak)

Example 70c: ¹H NMR (400 MHz, DMSO-d₆, ppm) δ 7.86 (d, J=2.4 Hz, 1H), 7.79 (d, J=1.6 Hz, 1H), 6.86-6.80 (m, 3H), 6.48 (s, 1H), 6.43 (q, J=7.0 Hz, 1H), 5.33 (s, 2H), 4.68-4.52 (m, 1H), 4.48-4.25 (m, 3H), 3.78-3.64 (m, 1H), 3.53-3.41 (m, 1H), 3.32-3.26 (m, 1H), 2.98-2.84 (m, 1H), 2.71-2.53 (m, 1H), 2.49-2.40 (m, 1H), 2.39-2.28 (m, 6H), 2.27-2.10 (m, 1H), 1.61 (d, J=6.8 Hz, 3H). LC-MS: (ESI, m/z): 683.3 [M+H]⁺ CHIRALPAK IC-3, 4.6×50 mm, 3 um; detected at 254 nm; (Hex:DCM=3:1) (0.1% DEA):EtOH=70:30; Flow rate: 1 mL/min; Retention time: 1.330 min (faster peak)

Example 70d: ¹H NMR (400 MHz, DMSO-d₆, ppm) δ 7.87 (d, J=2.4 Hz, 1H), 7.79 (d, J=1.6 Hz, 1H), 6.91-6.76 (m, 3H), 6.48 (s, 1H), 6.43 (q, J=6.9 Hz, 1H), 5.33 (s, 2H), 4.62-4.50 (m, 1H), 4.49-4.26 (m, 3H), 3.82-3.68 (m, 1H), 3.58-3.40 (m, 1H), 3.33-3.29 (m, 1H), 2.99-2.88 (m, 1H), 2.73-2.57 (m, 1H), 2.48-2.39 (m, 1H), 2.37 (d, J=2.2 Hz, 3H), 2.33 (s, 3H), 2.28-2.12 (m, 1H), 1.61 (d, J=7.0 Hz, 3H). LC-MS: (ESI, m/z): 683.4 [M+H]⁺ CHIRALPAK IC-3, 4.6×50 mm, 3 um; detected at 254 nm; (Hex:DCM=3:1) (0.1% DEA):EtOH=70:30; Flow rate: 1 mL/min; Retention time: 2.286 min (slower peak)

Example 71: 6-((R)-4-((R)-1-(2-aminopyridin-3-yl)ethyl)-8-chloro-2-((S)-1-((S)-4,4-difluoro-1-methylpyrrolidin-2-yl)ethoxy)-10-fluoro-5,6-dihydro-4H-[1,4]oxazepino[5,6,7-de]quinazolin-9-yl)-4-methyl-5-(trifluoromethyl)pyridin-2-amine

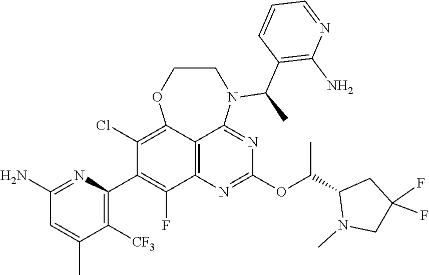

### Synthetic Route

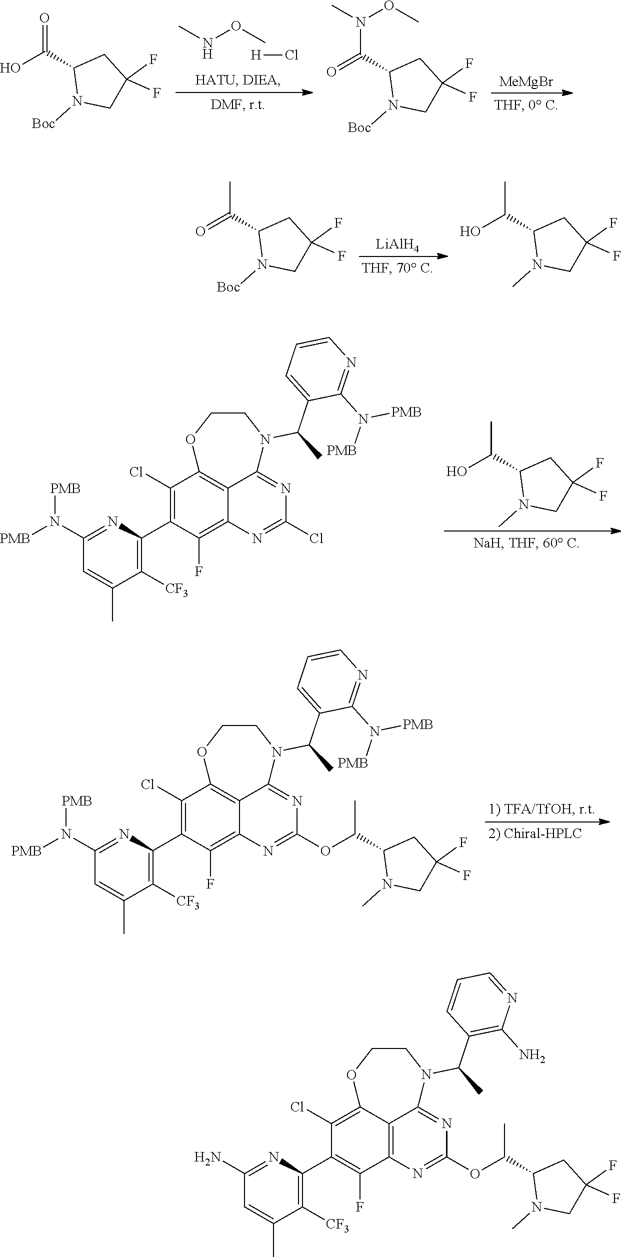

Step 1: tert-butyl (S)-4,4-difluoro-2-(methoxy(methyl)carbamoyl)pyrrolidine-1-carboxylate

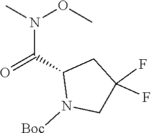 [SMILES: CON(C)C(=O)[C@@H]1CC(F)(F)CN1C(=O)OC(C)(C)C]

A solution of (S)-1-(tert-butoxycarbonyl)-4,4-difluoropyrrolidine-2-carboxylic acid (4.00 g, 15.92 mmol), N,N-diisopropylethylamine (12.35 g, 95.53 mmol) and N,O-dimethylhydroxylamine hydrochloride (4.66 g, 47.76 mmol) in N,N-dimethylformamide (25 mL) was stirred at 25° C. for 5 min. Then 2-(7-azabenzotriazol-1-yl)-N,N,N′,N′-tetramethyluronium hexafluorophosphate (7.26 g, 19.11 mmol) was added and stirred at 25° C. for 1 hour. After completion, the reaction mixture was diluted with water, extracted with ethyl acetate, washed with water and the organic layer was combined. The organic layer was dried over anhydrous sodium sulfate and concentrated under vacuum. The residue was purified by flash chromatography on reverse-phase column eluting with water/acetonitrile (7:3) to afford tert-butyl (S)-4,4-difluoro-2-(methoxy(methyl)carbamoyl)pyrrolidine-1-carboxylate (3.80 g, 11.10 mmol, 69.7% yield) as a light yellow oil. LC-MS: (ESI, m/z): 295.1 [M+H]⁺

Step 2: tert-butyl (S)-2-acetyl-4,4-difluoropyrrolidine-1-carboxylate

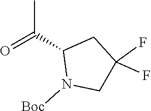 [SMILES: CC(=O)[C@@H]1CC(F)(F)CN1C(=O)OC(C)(C)C]

Under nitrogen, methylmagnesium bromide (41.62 mL, 41.62 mmol, 1 M in tetrahydrofuran) was added to a solution of tert-butyl (S)-4,4-difluoro-2-(methoxy(methyl)carbamoyl)pyrrolidine-1-carboxylate (3.50 g, 11.89 mmol) in tetrahydrofuran (40 mL) and stirred at 0° C. for 2 hours. After completion, the reaction was quenched with ammonium chloride saturated solution and concentrated under vacuum. The residue was purified by flash chromatography on reverse-phase column eluting with water/acetonitrile (1:1) to afford tert-butyl (S)-2-acetyl-4,4-difluoropyrrolidine-1-carboxylate (2.10 g, 7.92 mmol, 66.6% yield) as alight yellow oil. LC-MS: (ESI, m/z): 250.1 [M+H]⁺

Step 3: (S)-1-((S)-4,4-difluoro-1-methylpyrrolidin-2-yl)ethan-1-ol

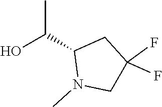

Lithiumaluminumhydride (502.5 mg, 13.24 mmol) was added to a solution of tert-butyl (S)-2-acetyl-4,4-difluoropyrrolidine-1-carboxylate (1.10 g, 4.41 mmol) in tetrahydrofuran (12 mL) at 0° C. and stirred at 70° C. for 1 hour. After completion, the reaction was quenched by sodium sulfate decahydrate. After stirring at 25° C. for 20 min, the mixture was filtered. The filtrate was dried over sodium sulfate and concentrated under reduced pressure to afford (S)-1-((S)-4,4-difluoro-1-methylpyrrolidin-2-yl)ethan-1-ol (300.1 mg, 1.62 mmol, 36.6% yield) as a light yellow oil. LC-MS: (ESI, m/): 166.1 [M+H]⁺

Step 4: (R)-6-(4-((R)-1-(2-(bis(4-methoxybenzyl)amino)pyridin-3-yl)ethyl)-8-chloro-2-((S)-1-((S)-4,4-difluoro-1-methylpyrrolidin-2-yl)ethoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

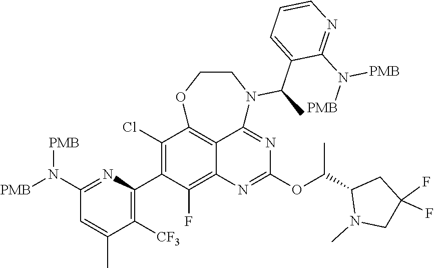

To a solution of (S)-1-((S)-4,4-difluoro-1-methylpyrrolidin-2-yl)ethan-1-ol (160.62 mg, 0.97 mmol) in tetrahydrofuran (10 mL) was added sodium hydride (114.4 mg, 2.86 mmol, 60% dispersion in mineral oil) at 0° C. and stirred at 25° C. for 30 min. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (600.0 mg, 0.57 mmol) was added at 25° C. and stirred at 60° C. for 1 hour. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with petroleum ether/ethyl acetate (4:1) to afford (R)-6-(4-((R)-1-(2-(bis(4-methoxybenzyl)amino)pyridin-3-yl)ethyl)-8-chloro-2-((S)-1-((S)-4,4-difluoro-1-methylpyrrolidin-2-yl)ethoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (180.2 mg, 0.13 mmol, 22.4% yield) as a light yellow solid. LC-MS: (ESI, m/z): 1177.4 [M+H]⁺

Step 5: 6-((R)-4-((R)-1-(2-aminopyridin-3-yl)ethyl)-8-chloro-2-((S)-1-((S)-4,4-difluoro-1-methylpyrrolidin-2-yl)ethoxy)-10-fluoro-5,6-dihydro-4H-[1,4]oxazepino[5,6,7-de]quinazolin-9-yl)-4-methyl-5-(trifluoromethyl)pyridin-2-amine

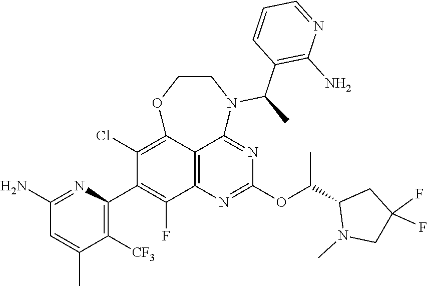

A solution of (R)-6-(4-((R)-1-(2-(bis(4-methoxybenzyl)amino)pyridin-3-yl)ethyl)-8-chloro-2-((S)-1-((S)-4,4-difluoro-1-methylpyrrolidin-2-yl)ethoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (160.0 mg, 0.12 mmol) in trifluoroacetic acid (5 mL) and trifluoromethanesulfonic acid (0.5 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 38% B to 63% B in 9 min, 63% B; Wave Length: 254/220 nm; RT1(min): 8.9 to afford 6-((R)-4-((R)-1-(2-aminopyridin-3-yl)ethyl)-8-chloro-2-((S)-1-((S)-4,4-difluoro-1-methylpyrrolidin-2-yl)ethoxy)-10-fluoro-5,6-dihydro-4H-[1,4]oxazepino[5,6,7-de]quinazolin-9-yl)-4-methyl-5-(trifluoromethyl)pyridin-2-amine (34.2 mg, 0.05 mmol, 36.1% yield). LC-MS: (ESI, m/z): 697.2 [M+H]⁺

Example 71: ¹H NMR (300 MHz, DMSO-d₆, ppm) δ 7.97 (dd, J=4.8, 1.6 Hz, 1H), 7.63 (dd, J=7.6, 1.6 Hz, 1H), 6.84 (s, 2H), 6.67 (q, J=4.8 Hz, 1H), 6.48 (s, 1H), 6.19 (d, J=6.8 Hz, 1H), 5.65 (s, 2H), 5.47-5.30 (m, 1H), 4.56-4.20 (m, 2H), 3.72-3.61 (m, 1H), 3.43-3.41 (m, 1H), 3.36-3.27 (m, 1H), 3.11-3.01 (m, 1H), 2.72-2.57 (m, 1H), 2.39-2.23 (m, 8H), 1.57 (d, J=6.4 Hz, 3H), 1.30 (d, J=6.4 Hz, 3H). LC-MS: (ESI, m/z): 697.2 [M+H]⁺

Example 72: 6-((R)-4-((R)-1-(2-aminopyridin-3-yl)ethyl)-8-chloro-2-((2-((S)-4,4-difluoro-1-methylpyrrolidin-2-yl)propan-2-yl)oxy)-10-fluoro-5,6-dihydro-4H-[1,4]oxazepino[5,6,7-de]quinazolin-9-yl)-4-methyl-5-(trifluoromethyl)pyridin-2-amine

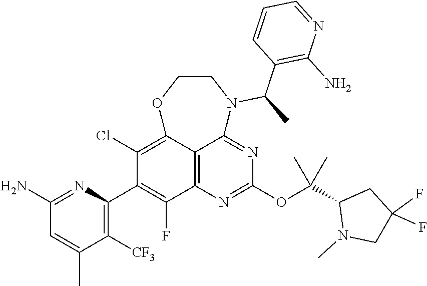

### Synthetic Route

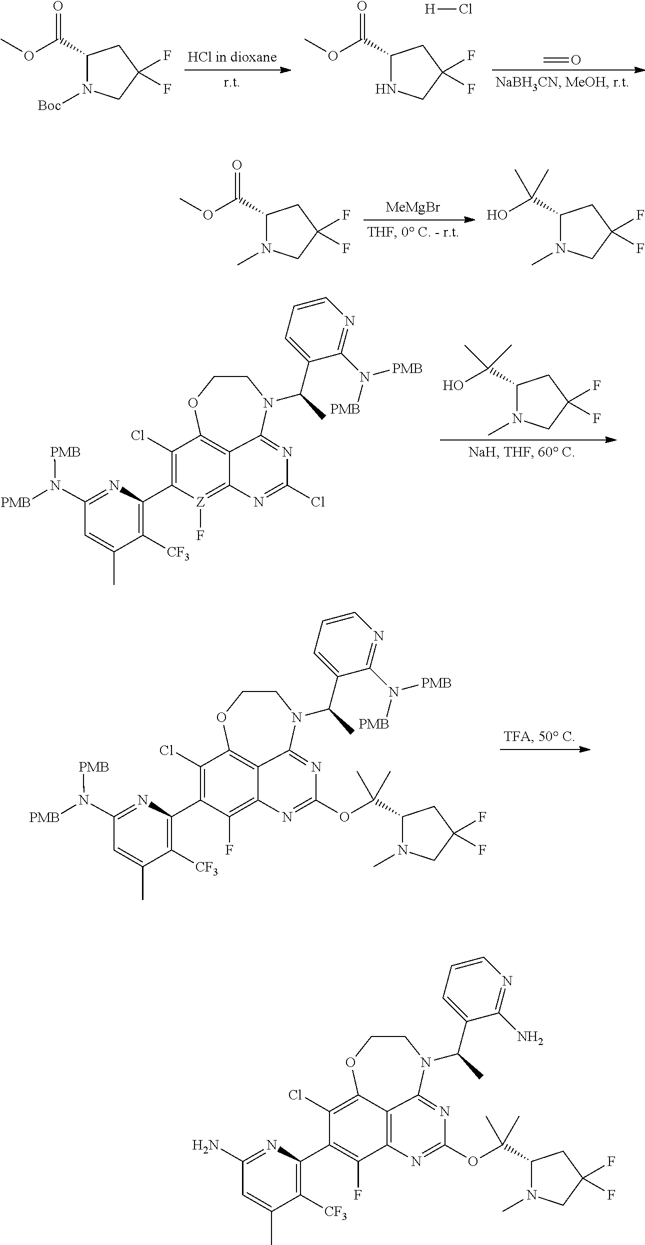

Step 1: methyl (S)-4,4-difluoropyrrolidine-2-carboxylate hydrochloride

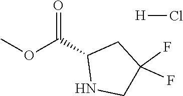

A solution of 1-(tert-butyl) 2-methyl (S)-4,4-difluoropyrrolidine-1,2-dicarboxylate (4.00 g, 15.08 mmol) in hydrogen chloride solution in 1,4-dioxane (30 mL, 120.00 mmol, 4.0 M) and was stirred for 1 hour at 25° C. After completion, the reaction mixture was concentrated under vacuum to afford methyl (S)-4,4-difluoropyrrolidine-2-carboxylate hydrochloride (3.00 g, 13.84 mmol, 91.8% yield) as a light brown solid. LC-MS: (ESI, m/z): 166.1 [M+H]⁺

Step 2: methyl (S)-4,4-difluoro-1-methylpyrrolidine-2-carboxylate

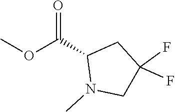

A solution of methyl (S)-4,4-difluoropyrrolidine-2-carboxylate hydrochloride (3.00 g, 13.84 mmol), formaldehyde in aqueous solution (6.74 g, 83.04 mmol, 37% purity) and acetic acid (166.2 mg, 2.77 mmol) in methyl alcohol (40 mL) was stirred at 25° C. for 10 min. Then sodiumcyanoborohydride (4.35 g, 69.20 mmol) was added and stirred at 25° C. for 1 hour. After completion, the reaction was quenched with water and concentrated under vacuum. The residue was purified by flash chromatography on silica gel eluting with petroleum ether/ethyl acetate (4:1) to afford methyl (S)-4,4-difluoro-1-methylpyrrolidine-2-carboxylate (2.40 g, 12.46 mmol, 90% yield) as a light yellow oil. LC-MS: (ESI, m/z): 180.1 [M+H]⁺

Step 3: (S)-2-(4,4-difluoro-1-methylpyrrolidin-2-yl)propan-2-ol

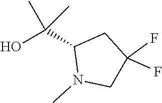

Under nitrogen, methylmagnesiumbromide in THE (15.63 mL, 15.63 mmol, 1 M in tetrahydrofuran) was added to a solution of methyl (S)-4,4-difluoro-1-methylpyrrolidine-2-carboxylate (800.0 mg, 4.47 mmol) in tetrahydrofuran (10 mL) and stirred at 25° C. for 2 hours. After completion, the reaction was quenched with ammonium chloride saturated solution and concentrated under vacuum. The residue was purified by flash chromatography on silica gel eluting with dichloromethane/methanol (10:1) to afford product to afford (S)-2-(4,4-difluoro-1-methylpyrrolidin-2-yl)propan-2-ol (400.5 mg, 2.03 mmol, 45.5% yield) as alight yellow oil. LC-MS: (ESI, m/z): 180.1 [M+H]⁺

Step 4: (R)-6-(4-((R)-1-(2-(bis(4-methoxybenzyl)amino)pyridin-3-yl)ethyl)-8-chloro-2-((2-((S)-4,4-difluoro-1-methylpyrrolidin-2-yl)propan-2-yl)oxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

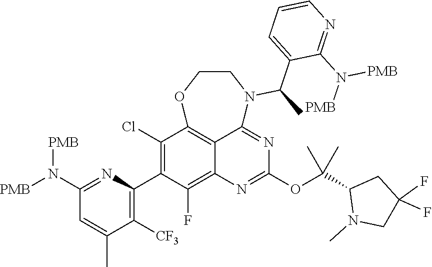

To a solution of (S)-2-(4,4-difluoro-1-methylpyrrolidin-2-yl)propan-2-ol (119.6 mg, 0.67 mmol) in tetrahydrofuran (3 mL) was added sodium hydride (133.5 mg, 3.34 mmol, 60% dispersion in mineral oil) at 0° C. and stirred at 25° C. for 30 min. Then (R)-6-(4-((R)-1-(2-(bis(4-methoxybenzyl)amino)pyridin-3-yl)ethyl)-8-chloro-2-((2-((S)-4,4-difluoro-1-methylpyrrolidin-2-yl)propan-2-yl)oxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (700.0 mg, 0.67 mmol) was added at 25° C. and stirred at 60° C. for 5 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with dichloromethane/methanol (93:7) to afford (R)-6-(4-((R)-1-(2-(bis(4-methoxybenzyl)amino)pyridin-3-yl)ethyl)-8-chloro-2-((2-((S)-4,4-difluoro-1-methylpyrrolidin-2-yl)propan-2-yl)oxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (580.2 mg, 0.37 mmol, 55.4% yield) as a white solid. LC-MS: (ESI, m/z): 1191.5 [M+H]⁺

Step 5: 6-((R)-4-((R)-1-(2-aminopyridin-3-yl)ethyl)-8-chloro-2-((2-((S)-4,4-difluoro-1-methylpyrrolidin-2-yl)propan-2-yl)oxy)-10-fluoro-5,6-dihydro-4H-[1,4]oxazepino[5,6,7-de]quinazolin-9-yl)-4-methyl-5-(trifluoromethyl)pyridin-2-amine

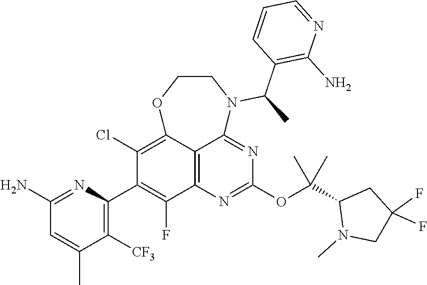

A solution of (R)-6-(4-((R)-1-(2-(bis(4-methoxybenzyl)amino)pyridin-3-yl)ethyl)-8-chloro-2-((2-((S)-4,4-difluoro-1-methylpyrrolidin-2-yl)propan-2-yl)oxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.17 mmol) in trifluoroacetic acid (5 mL) was stirred for 6 hours at 50° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 40% B to 65% B in 9 min, 65% B: Wave Length: 254/220 nm; RT1(min): 8.9 to afford 6-((R)-4-((R)-1-(2-aminopyridin-3-yl)ethyl)-8-chloro-2-((2-((S)-4,4-difluoro-1-methylpyrrolidin-2-yl)propan-2-yl)oxy)-10-fluoro-5,6-dihydro-4H-[1,4]oxazepino[5,6,7-de]quinazolin-9-yl)-4-methyl-5-(trifluoromethyl)pyridin-2-amine (37.6 mg, 0.05 mmol, 31.5% yield). LC-MS: (ESI, m/z): 711.3 [M+H]⁺

Example 72: ¹H NMR (300 MHz, DMSO-d₆, ppm) δ 7.97 (dd, J=5.1, 1.8 Hz, 1H), 7.63 (dd, J=7.5, 1.8 Hz, 1H), 6.81 (s, 2H), 6.66 (q, J=4.8 Hz, 1H), 6.48 (s, 1H), 6.18 (q, J=6.6 Hz, 1H), 5.71 (s, 2H), 4.50-4.39 (m, 1H), 4.37-4.22 (m, 1H), 3.77-3.48 (m, 2H), 3.44-3.39 (m, 1H), 3.35-3.26 (m, 1H), 2.87-2.65 (m, 1H), 2.51-2.32 (m, 7H), 2.28-2.02 (m, 1H), 1.75-1.47 (m, 9H). LC-MS: (ESI, m/z): 711.3 [M+H]⁺

Example 73a & 73b: 3-((R)-1-((R)-9-(3-amino-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-((S)-9-(3-amino-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

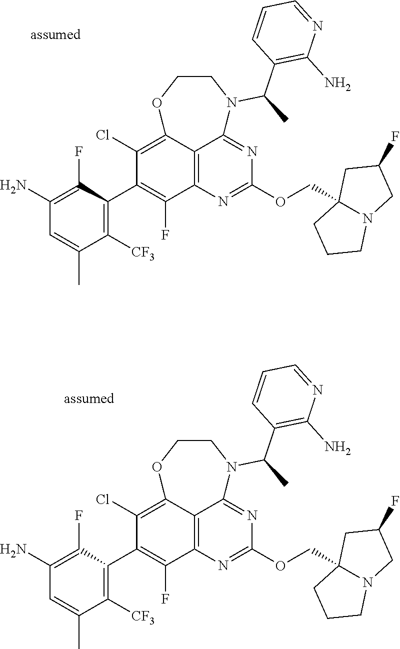

### Synthetic Route

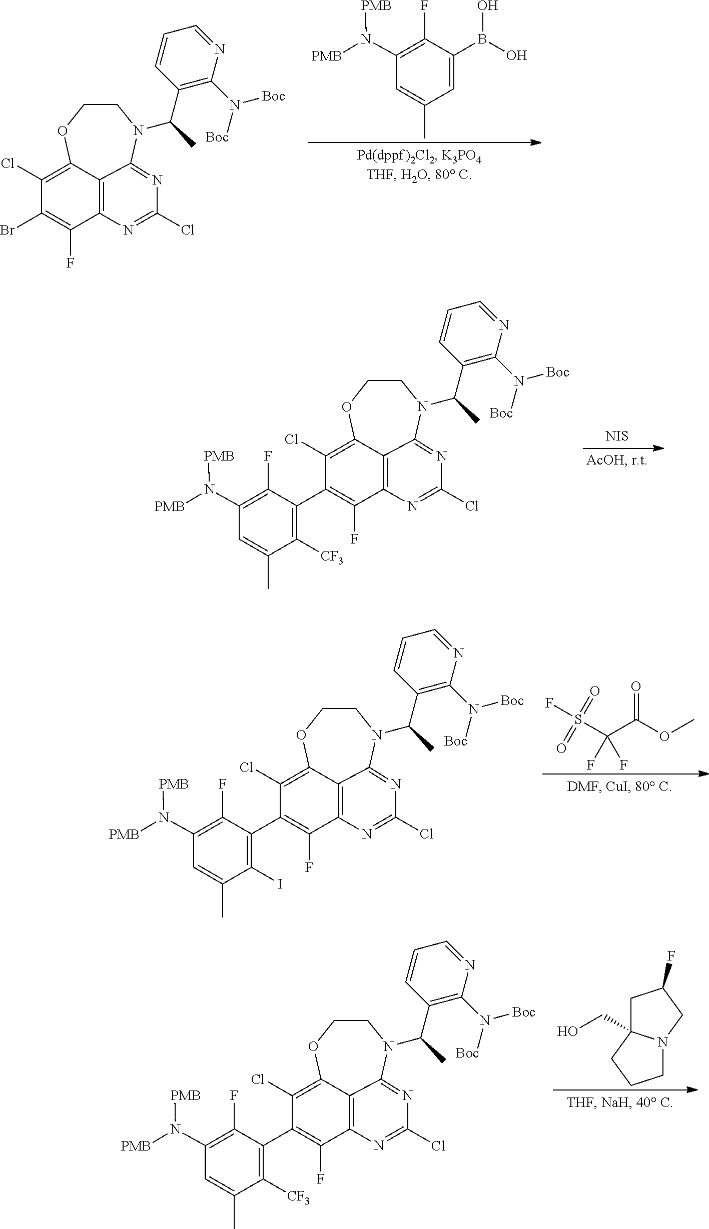

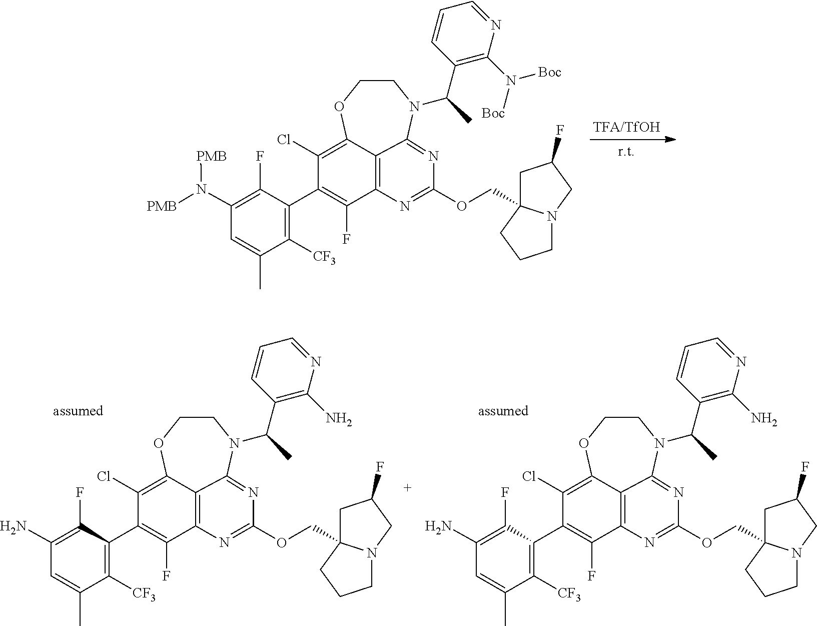

Step 1: tert-butyl N-[3-[(1R)-1-[7-[3-[bis[(4-methoxyphenyl)methyl]amino]-2-fluoro-5-methyl-phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate

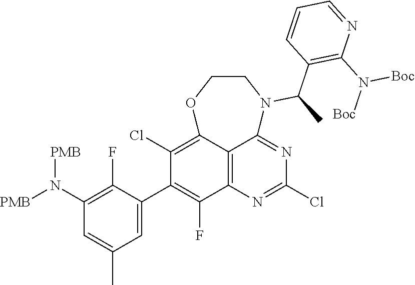

Under nitrogen, a solution of potassium phosphate tribasic (3.15 g, 14.85 mmol), [1,1′-bis(diphenylphosphino)ferrocene]dichloropalladium(II) (0.55 g, 0.74 mmol), tert-butyl N-[3-[(1R)-1-(7-bromo-3,8-dichloro-6-fluoro-10-oxa-2,4,13-triazatricyclo[7.4.1.05,14]tetradeca-1,3,5(14),6,8-pentaen-13-yl)ethyl]-2-pyridyl]-N-tert-butoxycarbonyl-carbamate (5.00 g, 7.43 mmol) and (3-(bis(4-methoxybenzyl)amino)-2-fluoro-5-methylphenyl)boronic acid (3.19 g, 7.80 mmol) in tetrahydrofuran (48 mL) and water (12 mL) was stirred at 80° C. for 1.5 hours. After completion, the reaction mixture was concentrated under vacuum, diluted with dichloromethane, washed with water and the organic layer was combined. The organic layer was dried over anhydrous sodium sulfate and concentrated under vacuum. The residue was purified by flash chromatography on silica gel eluting with petroleum ether/ethyl acetate (7:3) to afford tert-butyl N-[3-[(1R)-1-[7-[3-[bis[(4-methoxyphenyl)methyl]amino]-2-fluoro-5-methyl-phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (4.30 g, 3.73 mmol, 50.2% yield) as an orange solid. LC-MS: (ESI, m/z): 957.3 [M+H]⁺

Step 2: tert-butyl N-[3-[(1R)-1-[7-[3-[bis[(4-methoxyphenyl)methyl]amino]-2-fluoro-6-iodo-5-methyl-phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate

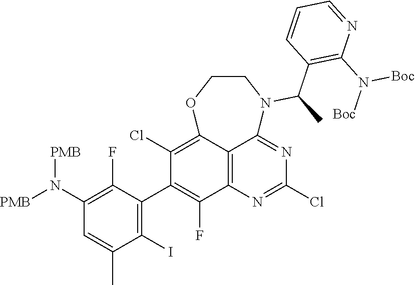

To a solution of tert-butyl N-[3-[(1R)-1-[7-[3-[bis[(4-methoxyphenyl)methyl]amino]-2-fluoro-5-methyl-phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (4.21 g, 3.64 mmol) in acetic acid (30 mL) was added N-iodosuccinimide (0.98 g, 4.37 mmol) at 0° C. and stirred at 25° C. for 30 min. After completion, the reaction mixture was quenched with sodium sulfite saturated solution, concentrated under vacuum, diluted with dichloromethane, washed with water, dried over anhydrous sodium sulfate and concentrated under vacuum. The residue was purified by flash chromatography on silica gel eluting with petroleum ether/ethyl acetate (7:3) to afford tert-butyl N-[3-[(1R)-1-[7-[3-[bis[(4-methoxyphenyl)methyl]amino]-2-fluoro-6-iodo-5-methyl-phenyl]-3,8-dichloro-6-fluoro-10-oxa-2,4,13-tiazatricyclo[7.4.1.05,14]tetradeca-1,3,5(14),6,8-pentaen-13-yl]ethyl]-2-pyridyl]-N-tert-butoxycarbonyl-carbamate (3.30 g, 2.62 mmol, 72% yield) as a yellow solid. LC-MS: (ESI, m/z): 1083.2 [M+H]⁺

Step 3: tert-butyl N-[3-[(1R)-1-[7-[3-[bis[(4-methoxyphenyl)methyl]amino]-2-fluoro-5-methyl-6-(trifluoromethyl)phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate

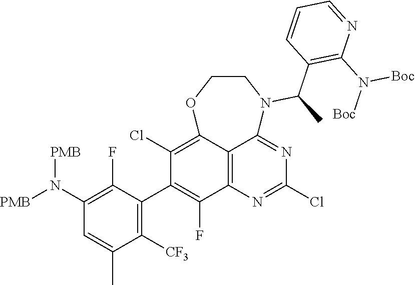

Under nitrogen, a solution of copper(I) iodide (4.99 g, 26.19 mmol), methyl 2,2-difluoro-2-(fluorosulfonyl)acetate (12.58 g, 65.47 mmol) and tert-butyl N-[3-[(1R)-1-[7-[3-[bis[(4-methoxyphenyl)methyl]amino]-2-fluoro-6-iodo-5-methyl-phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (3.30 g, 2.62 mmol) in N,N-dimethylformamide (60 mL) was stirred at 80° C. for 70 min. After completion, the reaction mixture was diluted with water, extracted with ethyl acetate, washed with water and the organic layer was combined. The organic layer was dried over anhydrous sodium sulfate and concentrated under vacuum. The residue was purified by flash chromatography on silica gel eluting with petroleum ether/ethyl acetate (1:1) to afford tert-butyl N-[3-[(1R)-1-[7-[3-[bis[(4-methoxyphenyl)methyl]amino]-2-fluoro-5-methyl-6-(trifluoromethyl)phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (1.50 g, 1.24 mmol, 47.5% yield) as a yellow oil. LC-MS: (ESI, m/z): 1025.3 [M+H]⁺

Step 4: tert-butyl (3-((1R)-1-(9-(3-(bis(4-methoxybenzyl)amino)-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate

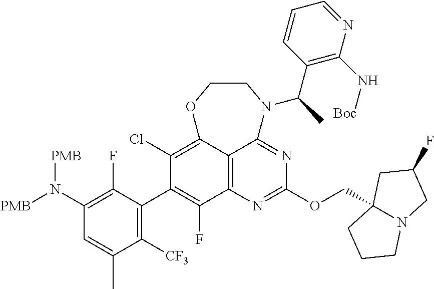

To a solution of ((2R,7aS)-2-fluorotetrahydro-1H-pyrrolizin-7a(5H)-yl)methanol (382.5 mg, 2.40 mmol) in tetrahydrofuran (20 mL) was added sodium hydride (96.1 mg, 2.40 mmol, 60% dispersion in mineral oil) at 0° C. and stirred at 25° C. for 30 min. Then tert-butyl N-[3-[(1R)-1-[7-[3-[bis[(4-methoxyphenyl)methyl]amino]-2-fluoro-5-methyl-6-(trifluoromethyl)phenyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (1.45 g, 1.20 mmol) was added at 25° C. and stirred at 40° C. for 1.5 hours. After completion, the reaction was quenched with ammonium chloride saturated solution at 0° C., concentrated under vacuum, diluted with dichloromethane, washed with water, dried over anhydrous sodium sulfate and concentrated under vacuum. The residue was purified by flash chromatography on silica gel eluting with dichloromethane/methanol (93:7) to afford tert-butyl (3-((1R)-1-(9-(3-(bis(4-methoxybenzyl)amino)-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (1.05 g, 0.77 mmol, 64.2% yield) as a white solid. LC-MS: (ESI, m/z): 1048.4 [M+H]⁺

Step 5: 3-((R)-1-((R)-9-(3-amino-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-((S)-9-(3-amino-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

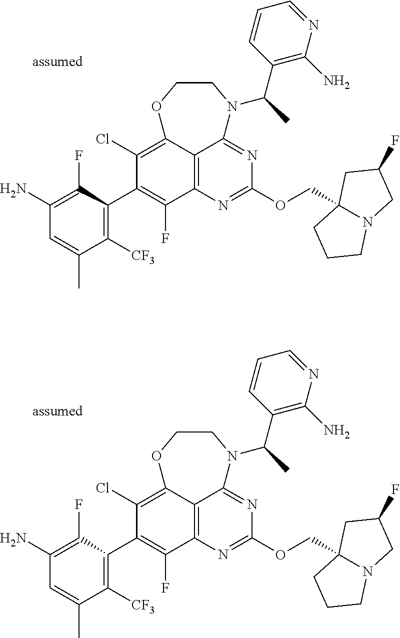

A solution of tert-butyl (3-((1R)-1-(9-(3-(bis(4-methoxybenzyl)amino)-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (1.05 g, 0.77 mmol) in trifluoroacetic acid (10 mL) and trifluoromethanesulfonic acid (1 mL) was stirred for 5 hours at 25° C. After completion, the reaction mixture was diluted with dichloromethane and adjusted to pH >7 with sodium bicarbonate saturated solution, extracted with dichloromethane, and the organic layer was combined. The organic layer was dried over anhydrous sodium sulfate and concentrated under vacuum. The residue was purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 45% B to 58% B in 12 min, 58% B; Wave Length: 220/254 nm; RT1(min): 8.38 to afford two diastereoisomers. The faster peak was further purified by Prep-HPLC with the following conditions: Column: XSelect CSH Fluoro Phenyl, 30*150 mm, 5 μm; Mobile Phase A: water (0.1% FA), Mobile Phase B: acetonitrile; Flow rate: 60 mL/min; Gradient: 5% B to 35% B in 7 min; Wave Length: 254/220 nm; RT1(min): 5.4 to afford 3-((R)-1-((R)-9-(3-amino-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine (35.2 mg, 0.05 mmol, 13.5% yield). The slower peak was further purified by Chiral-Prep-HPLC with the following conditions: Column: CHIRALPAK IF, 2*25 cm, 5 μm; Mobile Phase A: Hex:DCM=3:1(0.5% 2M NH₃-MeOH)-HPLC, Mobile Phase B: EtOH-HPLC; Flow rate: 20 mL/min; Gradient: 85% B to 85% B in 10.5 min; Wave Length: 220/254 nm; RT1(min): 6.316; Sample Solvent: EtOH-HPLC to afford 3-((R)-1-((S)-9-(3-amino-2-fluoro-5-methyl-6-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine (62.6 mg, 0.09 mmol, 23.8% yield). LC-MS: (ESI, m/z): 708.2 [M+H]⁺

Example 73a: ¹H NMR (300 MHz, Methanol-d₄, ppm) δ 7.95 (dd, J=4.8, 1.2 Hz, 1H), 7.75 (d, J=7.2 Hz, 1H), 6.86 (d, J=8.7 Hz, 1H), 6.78 (dd, J=7.2, 5.1 Hz, 1H), 6.51 (q, J=6.6 Hz, 1H), 5.36 (d, J=53.7 Hz, 1H), 4.46-4.32 (m, 1H), 4.38 (d, J=3.9 Hz, 2H), 4.34-4.20 (m, 1H), 3.70 (dd, J=15.6, 6.6 Hz, 1H), 3.54-3.41 (m, 2H), 3.38-3.32 (m, 2H), 3.18-3.05 (m, 1H), 2.45-2.31 (m, 4H), 2.28-2.20 (m, 2H), 2.14-1.93 (m, 3H), 1.65 (d, J=6.9 Hz, 3H). LC-MS: (ESI, m/z): 708.2 [M+H]⁺

Example 73b: ¹H NMR (300 MHz, DMSO-d₆, ppm) δ 7.97 (d, J=4.8, 1H), 7.63 (d, J=6.9 Hz, 1H), 6.83 (d, J=8.7 Hz, 1H), 6.66 (dd, J=7.2, 4.8 Hz, 1H), 6.29 (q, J=6.6 Hz, 1H), 6.00 (s, 2H), 5.75 (s, 2H), 5.28 (d, J=53.7 Hz, 1H), 4.55-4.39 (m, 1H), 4.35-4.17 (m, 1H), 4.09 (s, 2H), 3.69 (dd, J=15.9, 7.2 Hz, 1H), 3.42-3.33 (m, 1H), 3.15-3.03 (m, 2H), 2.99 (s, 1H), 2.85-2.78 (m, 1H), 2.38-2.33 (m, 3H), 2.21-2.09 (m, 1H), 2.08-1.94 (m, 2H), 1.85-1.77 (m, 3H), 1.57 (d, J=6.6 Hz, 3H). LC-MS: (ESI, m/z): 708.2 [M+H]⁺

Example 74a & 74b: 3-((R)-1-((S)-9-(3-amino-2-fluoro-6-iodo-5-methylphenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-((R)-9-(3-amino-2-fluoro-6-iodo-5-methylphenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

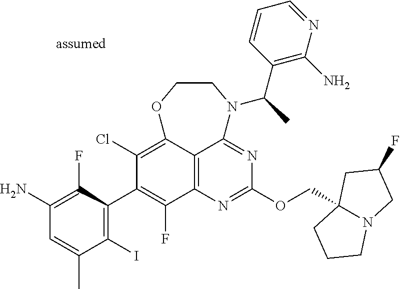

### Synthetic Route

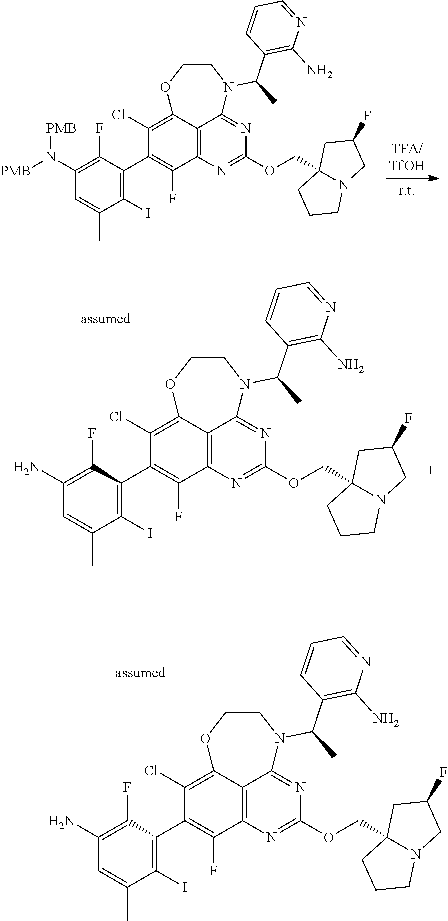

Step 1: 3-((R)-1-((S)-9-(3-amino-2-fluoro-6-iodo-5-methylphenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-((R)-9-(3-amino-2-fluoro-6-iodo-5-methylphenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

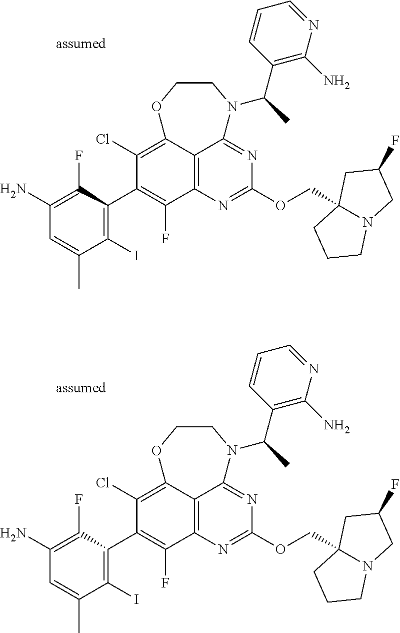

A solution of 3-((1R)-1-(9-(3-(bis(4-methoxybenzyl)amino)-2-fluoro-6-iodo-5-methylphenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine (150.0 mg, 0.15 mmol) in trifluoroacetic acid (5 mL) and trifluoromethanesulfonic acid (0.5 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 43% B to 73% B in 10 min, 73% B: Wave Length: 220/254 nm; RT1(min): 6.58 to afford 3-((R)-1-((S)-9-(3-amino-2-fluoro-6-iodo-5-methylphenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine (20.9 mg, 0.03 mmol, 17.3% yield) and 3-((R)-1-((R)-9-(3-amino-2-fluoro-6-iodo-5-methylphenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine (21.7 mg, 0.03 mmol, 18.1% yield). LC-MS: (ESI, m/z): 766.1 [M+H]⁺

Example 74a: ¹H NMR (300 MHz, DMSO-d₆, ppm) δ 7.97 (dd, J=4.8, 1.2 Hz, 1H), 7.64 (d, J=6.9 Hz, 1H), 6.91 (d, J=9.0 Hz, 1H), 6.67 (dd, J=7.5, 5.1 Hz, 1H), 6.33 (q, J=6.9 Hz, 1H), 5.75 (s, 2H), 5.52-5.11 (m, 3H), 4.58-4.19 (m, 2H), 4.18-4.03 (m, 2H), 3.82-3.61 (m, 1H), 3.58-3.53 (m, 1H), 3.19-2.93 (m, 3H), 2.95-2.72 (m, 1H), 2.32 (s, 3H), 2.25-1.95 (m, 3H), 1.94-1.73 (m, 3H), 1.57 (d, J=6.6 Hz, 3H). LC-MS: (ESI, m/z): 766.1 [M+H]⁺

Example 74b: ¹H NMR (300 MHz, DMSO-d₆, ppm) δ 7.97 (dd, J=4.9, 1.7 Hz, 1H), 7.63 (d, J=6.6 Hz, 1H), 6.91 (d, J=9.0 Hz, 1H), 6.67 (dd, J=7.5, 4.8 Hz, 1H), 6.32 (q, J=6.9 Hz, 1H), 5.76 (s, 2H), 5.50-5.14 (m, 3H), 4.59-4.24 (m, 2H), 4.15-4.00 (m, 2H), 3.82-3.61 (m, 1H), 3.53-3.40 (m, 1H), 3.17-3.02 (m, 2H), 3.00 (s, 1H), 2.91-2.74 (m, 1H), 2.32 (s, 3H), 2.21-2.13 (m, 1H), 2.12-1.96 (m, 2H), 1.93-1.70 (m, 3H), 1.58 (d, J=6.9 Hz, 3H). LC-MS: (ESI, m/z): 766.1 [M+H]⁺

Example 75a & 75b: 6-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-4-methyl-5-(trifluoromethyl)pyridin-2-amine & 6-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-4-methyl-5-(trifluoromethyl)pyridin-2-amine

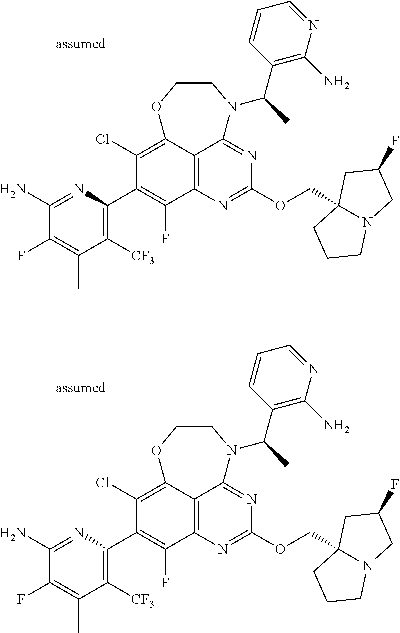

### Synthetic Route

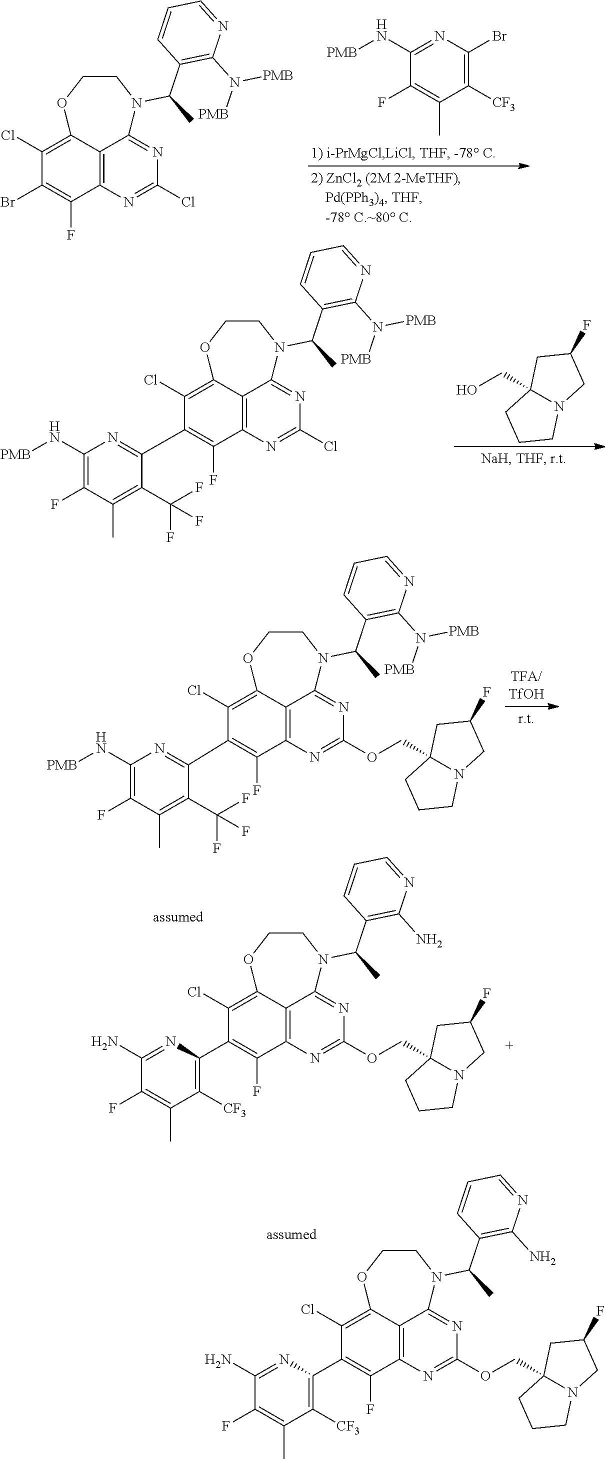

Step 1: 6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-3-fluoro-N-(4-methoxybenzyl)-4-methyl-5-(trifluoromethyl)pyridin-2-amine

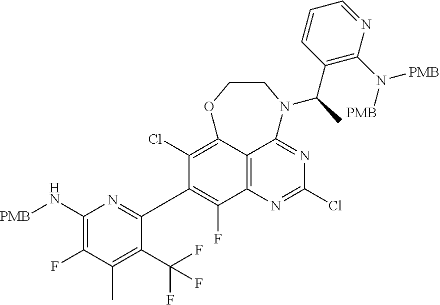

Under nitrogen, to a solution of (R)-3-(1-(9-bromo-2,8-dichloro-10-fluoro-5,6-dihydro-4H-[1,4]oxazepino[5,6,7-de]quinazolin-4-yl)ethyl)-N,N-bis(4-methoxybenzyl)pyridin-2-amine (800.0 mg, 1.12 mmol) in tetrahydrofuran (10 mL) was added isopropylmagnesium chloride-lithium chloride complex (1.04 mL, 1.35 mmol, 1.3 M in tetrahydrofuran) at −78° C. and the reaction was stirred at −78° C. for 20 min. Then zinc chloride (1.68 mL, 3.36 mmol, 2 M in 2-methyltetrahydrofuran) was added at −78° C. The reaction was stirred at −78° C. for 5 min and stirred at 25° C. for 20 min. The mixture was transferred into a solution of tetrakis(triphenylphosphine)palladium (194.42 mg, 0.17 mmol) and 6-bromo-3-fluoro-N-(4-methoxybenzyl)-4-methyl-5-(trifluoromethyl)pyridin-2-amine (441.0 mg, 1.12 mmol) in tetrahydrofuran (10 mL) and stirred at 80° C. for 1 hour. After completion, the reaction mixture was diluted with ethyl acetate, washed with water and the organic layer was combined. The organic layer was dried over anhydrous sodium sulfate and concentrated under vacuum. The residue was purified by flash chromatography on silica gel eluting with petroleum ether/ethyl acetate (7:3) to afford 6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-3-fluoro-N-(4-methoxybenzyl)-4-methyl-5-(trifluoromethyl)pyridin-2-amine (700.0 mg, 0.73 mmol, 65.9% yield) as a yellow solid. LC-MS: (ESI, m/z): 946.3 [M+H]⁺

Step 2: 6-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-N-(4-methoxybenzyl)-4-methyl-5-(trifluoromethyl)pyridin-2-amine

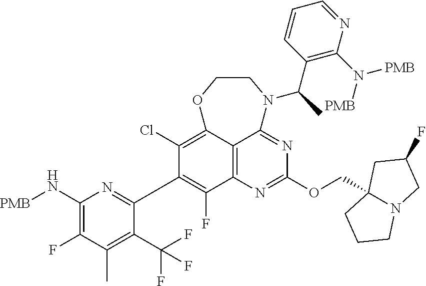

To a solution of ((2R,7aS)-2-fluorotetrahydro-1H-pyrrolizin-7a(5H)-yl)methanol (302.6 mg, 1.90 mmol) in tetrahydrofuran (10 mL) was added sodium hydride (76.1 mg, 3.17 mmol, 60% dispersion in mineral oil) at 0° C. and stirred at 0° C. for 30 min. Then the reaction mixture was added to a solution of 6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-3-fluoro-N-(4-methoxybenzyl)-4-methyl-5-(trifluoromethyl)pyridin-2-amine (600.0 mg, 0.63 mmol) in tetrahydrofuran (10 mL) at 0° C. and stirred at 25° C. for 4 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with petroleum ether/ethyl acetate (75:25) to afford 6-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-N-(4-methoxybenzyl)-4-methyl-5-(trifluoromethyl)pyridin-2-amine (500.3 mg, 0.47 mmol, 73.8% yield) as a yellow solid. LC-MS: (ESI, m/z): 1069.4 [M+H]⁺

Step 3: 6-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-4-methyl-5-(trifluoromethyl)pyridin-2-amine & 6-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-4-methyl-5-(trifluoromethyl)pyridin-2-amine

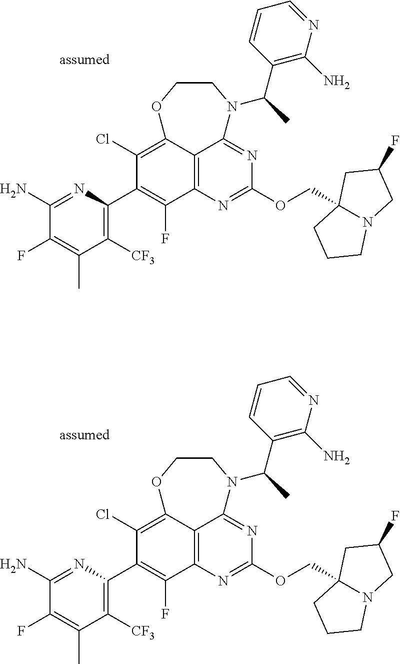

A solution of 6-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-N-(4-methoxybenzyl)-4-methyl-5-(trifluoromethyl)pyridin-2-amine (400.0 mg, 0.37 mmol) in trifluoroacetic acid (5 mL) and trifluoromethanesulfonic acid (0.5 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 17% B to 60% B in 9 min, 60% B; Wave Length: 254/220 nm; RT1(min): 8.9 to afford 6-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-4-methyl-5-(trifluoromethyl)pyridin-2-amine (60.3 mg, 0.08 mmol, 22.7% yield) and 6-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-3-fluoro-4-methyl-5-(trifluoromethyl)pyridin-2-amine (50.3 mg, 0.07 mmol, 19% yield). LC-MS: (ESI, m/z): 709.2 [M+H]⁺

Example 75a: ¹H NMR (300 MHz, DMSO-d₆, ppm) δ 7.98 (dd, J=4.9, 1.7 Hz, 1H), 7.63 (dd, J=7.6, 1.8 Hz, 1H), 7.19 (s, 2H), 6.67 (q, J=4.9 Hz, 1H), 6.26 (q, J=6.6 Hz, 1H), 5.73 (s, 2H), 5.29 (d, J=54.0 Hz, 1H), 4.56-4.36 (m, 1H), 4.35-4.18 (m, 1H), 4.08 (q, J=10.3 Hz, 2H), 3.75-3.55 (m, 1H), 3.46-3.34 (m, 1H), 3.24-3.02 (m, 2H), 3.06-2.97 (m, 1H), 2.92-2.70 (m, 1H), 2.44-2.25 (m, 3H), 2.23-2.09 (m, 1H), 2.07-2.01 (m, 2H), 1.93-1.66 (m, 3H), 1.57 (d, J=6.8 Hz, 3H). LC-MS: (ESI, m/z): 709.2 [M+H]⁺

Example 76b: ¹H NMR (300 MHz, DMSO-d₆, ppm) δ 7.96 (dd, J=4.9, 1.7 Hz, 1H), 7.63 (dd, J=7.5 Hz, 1H), 7.19 (s, 2H), 6.66 (q, J=4.9 Hz, 1H), 6.28 (q, J=6.8 Hz, 1H), 5.80 (s, 2H), 5.28 (d, J=54.4 Hz, 1H), 4.55-4.42 (m, 1H), 4.36-4.21 (m, 1H), 4.07 (s, 2H), 3.76-3.59 (m, 1H), 3.44-3.34 (m, 1H), 3.18-3.01 (m, 2H), 3.00 (s, 1H), 2.87-2.71 (m, 1H), 2.34 (s, 3H), 2.19-2.10 (m, 1H), 2.05-1.89 (m, 2H), 1.89-1.71 (m, 3H), 1.58 (d, J=6.8 Hz, 3H). LC-MS: (ESI, m/z): 709.2 [M+H]⁺

Example 76a & 76b: 3-((R)-1-(9-((S)-5-amino-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-(9-((R)-5-amino-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

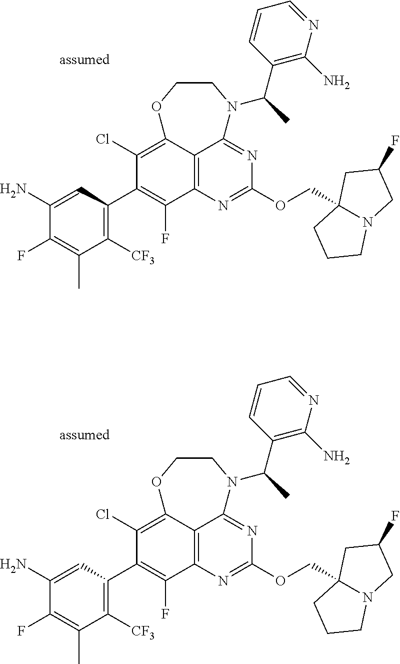

### Synthetic Route

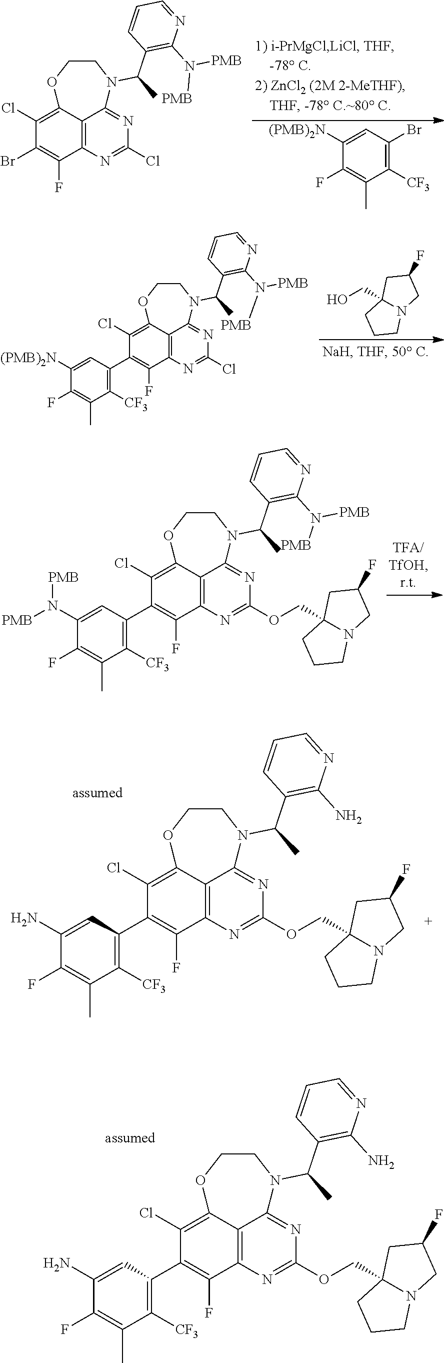

Step 1: 3-((1R)-1-(9-(5-(bis(4-methoxybenzyl)amino)-4-fluoro-3-methyl-2-(trifluoromethyl)phenyl)-2,8-dichloro-10-fluoro-5,6-dihydro-4H-[1,4]oxazepino[5,6,7-de]quinazolin-4-yl)ethyl)-N,N-bis(4-methoxybenzyl)pyridin-2-amine

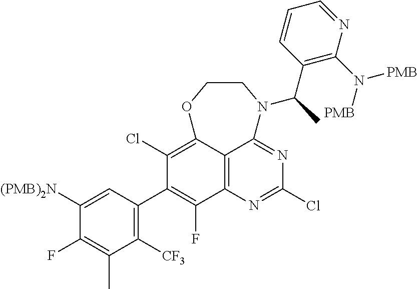

Under nitrogen, to a solution of (R)-3-(1-(9-bromo-2,8-dichloro-10-fluoro-5,6-dihydro-4H-[1,4]oxazepino[5,6,7-de]quinazolin-4-yl)ethyl)-N,N-bis(4-methoxybenzyl)pyridin-2-amine (2.40 g, 3.36 mmol) in tetrahydrofuran (25 mL) was added isopropylmagnesium chloride-lithium chloride complex (3.11 mL, 4.04 mmol, 1.3 M in tetrahydrofuran) at −78° C. and the reaction was stirred at −78° C. for 20 min. Then zinc chloride (5.05 mL, 10.09 mmol, 2 M in 2-methyltetrahydrofuran) was added at −78° C. The reaction was stirred at −78° C. for 5 min and stirred at 25° C. for 20 min. The mixture was transferred into a solution of tetrakis(triphenylphosphine) palladium (0.58 g, 0.50 mmol) and 5-bromo-2-fluoro-N,N-bis(4-methoxybenzyl)-3-methyl-4-(trifluoromethyl)aniline (1.72 g, 3.36 mmol) in tetrahydrofuran (10 mL) and stirred at 80° C. for 1 hour. After completion, the reaction mixture was diluted with ethyl acetate, washed with water and the organic layer was combined. The organic layer was dried over anhydrous sodium sulfate and concentrated under vacuum. The residue was purified by flash chromatography on silica gel eluting with petroleum ether/ethyl acetate (7:3) to afford 3-((1R)-1-(9-(5-(bis(4-methoxybenzyl)amino)-4-fluoro-3-methyl-2-(trifluoromethyl)phenyl)-2,8-dichloro-10-fluoro-5,6-dihydro-4H-[1,4]oxazepino[5,6,7-de]quinazolin-4-yl)ethyl)-N,N-bis(4-methoxybenzyl)pyridin-2-amine (620.4 mg, 0.58 mmol, 17.3% yield) as a yellow solid. LC-MS: (ESI, m/z): 1064.3 [M+H]⁺.

Step 2: 3-((1R)-1-(9-(5-(bis(4-methoxybenzyl)amino)-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N,N-bis(4-methoxybenzyl)pyridin-2-amine

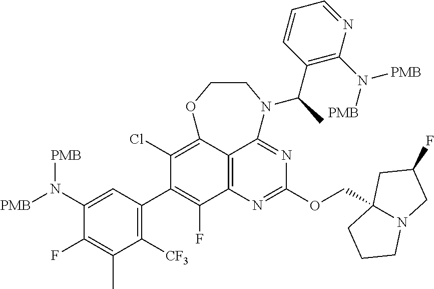

To a solution of ((2R,7aS)-2-fluorotetrahydro-1H-pyrrolizin-7a(5H)-yl)methanol (277.8 mg, 1.74 mmol) in tetrahydrofuran (10 mL) was added sodium hydride (83.7 mg, 3.49 mmol, 60% dispersion in mineral oil) at 0° C. and stirred at 0° C. for 30 min. Then the reaction mixture was added to a solution of 3-((1R)-1-(9-(5-(bis(4-methoxybenzyl)amino)-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N,N-bis(4-methoxybenzyl)pyridin-2-amine (620.0 mg, 0.58 mmol) in tetrahydrofuran (5 mL) at 0° C. and stirred at 50° C. for 5 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with petroleum ether/ethyl acetate (70:30) to afford 3-((1R)-1-(9-(5-(bis(4-methoxybenzyl)amino)-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N,N-bis(4-methoxybenzyl)pyridin-2-amine (311.4 mg, 0.26 mmol, 45.1% yield) as a yellow solid. LC-MS: (ESI, m/z): 1188.5 [M+H]⁺

Step 3: 3-((R)-1-(9-((S)-5-amino-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-(9-((R)-5-amino-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

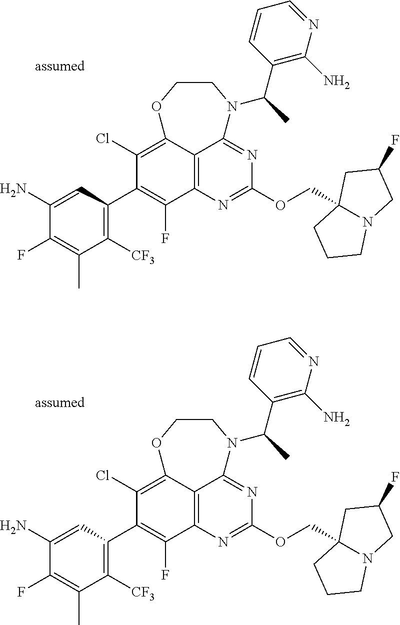

A solution of 3-((1R)-1-(9-(5-(bis(4-methoxybenzyl)amino)-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N,N-bis(4-methoxybenzyl)pyridin-2-amine (300.0 mg, 0.25 mmol) in trifluoroacetic acid (5 mL) and trifluoromethanesulfonic acid (0.5 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with petroleum ether/ethyl acetate (1:1) to afford product. The product was further purified by Prep-HPLC with the following conditions: Column: XBridge Prep OBD C18 Column, 30*150 mm, 5 μm; Mobile Phase A: water (10 mmol/L NH₄HCO₃), Mobile Phase B: ACN; Flow rate: 60 mL/min; Gradient: 41% B to 66% B in 9 min, 66% B; Wave Length: 254/220 nm; RT1(min): 8.6; Number Of Runs: 0 to afford 3-((R)-1-(9-((S)-5-amino-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine (20.4 mg, 0.02 mmol, 11.4% yield) and 3-((R)-1-(9-((R)-5-amino-4-fluoro-3-methyl-2-(trifluoromethyl)phenyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine (24.3 mg, 0.03 mmol, 13.6% yield). LC-MS: (ESI, m/z): 708.2 [M+H]⁺

Example 76a: ¹H NMR (300 MHz, DMSO-d₆, ppm) δ 7.96 (dd, J=4.9, 1.7 Hz, 1H), 7.61 (dd, J=7.5, 1.8 Hz, 1H), 6.65 (q, J=4.9 Hz, 1H), 6.37 (d, J=8.5 Hz, 1H), 6.27 (q, J=6.7 Hz, 1H), 6.01 (s, 2H), 5.74 (s, 2H), 5.27 (d, J=54.4 Hz, 1H), 4.51-4.39 (m, 1H), 4.31-4.19 (m, 1H), 4.08 (q, J=8.5 Hz, 2H), 3.73-3.60 (m, 1H), 3.43-3.32 (m, 1H), 3.16-3.08 (m, 2H), 3.04-2.90 (m, 1H), 2.88-2.74 (m, 1H), 2.40-2.23 (m, 3H), 2.23-1.97 (m, 3H), 1.90-1.71 (m, 3H), 1.54 (d, J=6.8 Hz, 3H). LC-MS: (ESI, m/z): 708.2 [M+H]⁺

Example 76b: ¹H NMR (300 MHz, DMSO-d₆, ppm) δ 7.95 (dd, J=4.9, 1.7 Hz, 1H), 7.61 (dd, J=7.5, 1.8 Hz, 1H), 6.65 (q, J=7.5, 4.9 Hz, 1H), 6.37 (d, J=8.6 Hz, 1H), 6.28 (q, J=6.8 Hz, 1H), 6.01 (s, 2H), 5.77 (s, 2H), 5.31 (d, J=54.1 Hz, 1H), 4.59-4.42 (m, 1H), 4.34-4.22 (m, 1H), 4.14 (s, 2H), 3.76-3.57 (m, 1H), 3.50-3.35 (m, 1H), 3.24-3.00 (m, 3H), 2.88 (s, 1H), 2.42-2.27 (m, 3H), 2.26-2.14 (m, 1H), 2.14-1.98 (m, 2H), 1.90-1.72 (m, 3H), 1.56 (d, J=6.8 Hz, 3H). LC-MS: (ESI, m/z): 708.2 [M+H]⁺

Example 77a & 77b & 77c & 77d: 4-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine & 4-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine & 4-((1S)-1-((9R)-9-(6-amino-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine

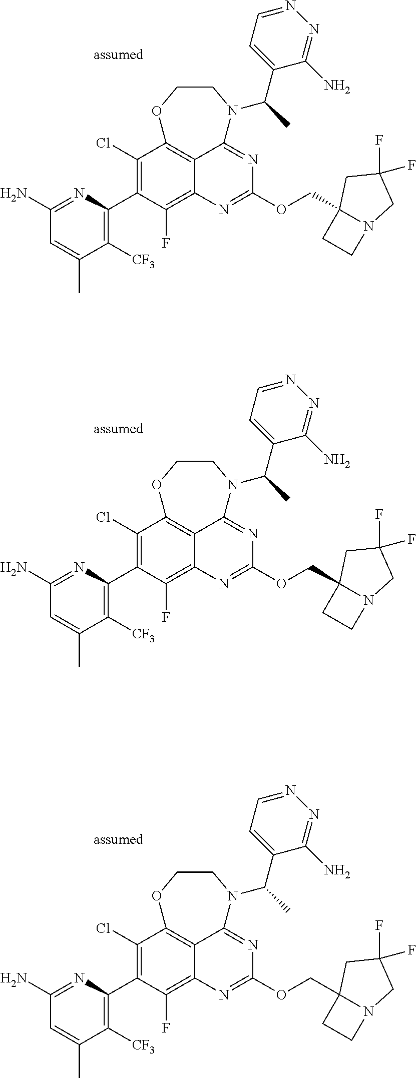

### Synthetic Route

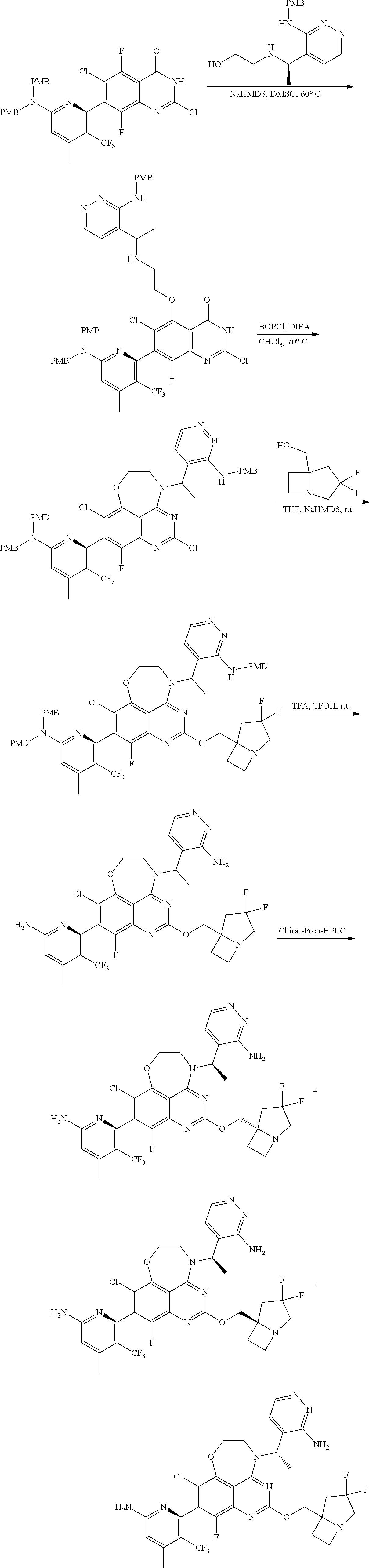

Step 1: (7R)-7-(6-(bis(4-methoxybenzyl)amino)-4-methyl-3-(trifluoromethyl)pyridin-2-yl)-2,6-dichloro-8-fluoro-5-(2-((1-(3-((4-methoxybenzyl)amino)pyridazin-4-yl)ethyl)amino)ethoxy)quinazolin-4(3H)-one

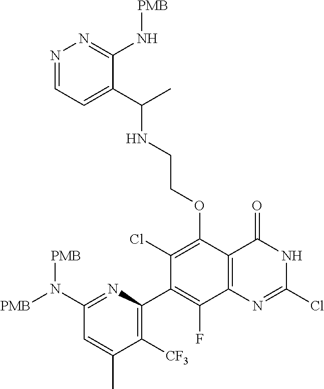

Similar to as described in General Procedure A. Under nitrogen, sodium bis(trimethylsilyl)amide (13.5 mL, 13.52 mmol, 1 M in tetrahydrofuran) was added to a solution of (R)-2-((1-(3-((4-methoxybenzyl)amino)pyridazin-4-yl)ethyl)amino)ethan-1-ol (1.36 g, 2.57 mmol) in dimethyl sulfoxide (10 mL) and stirred for 20 min at 60° C. Then the reaction mixture was added to a solution of (R)-7-(6-(bis(4-methoxybenzyl)amino)-4-methyl-3-(trifluoromethyl)pyridin-2-yl)-2,6-dichloro-5,8-difluoroquinazolin-4(3H)-one (3.00 g, 1.43 mmol) in dimethyl sulfoxide (20 mL) and stirred at 60° C. for 2 hours. After completion, the reaction was quenched with saturated ammonium chloride solution, diluted with water, extracted with ethyl acetate, washed with water and the organic layer was combined. The organic layer was dried over anhydrous sodium sulfate and concentrated under vacuum. The residue was purified by a reversed-phase chromatography directly with the following conditions: Column, C18 silica gel; mobile phase, A: water, B: acetonitrile, B % (45%-50% in 15 min): Detector, UV 254 nm to afford (7R)-7-(6-(bis(4-methoxybenzyl)amino)-4-methyl-3-(trifluoromethyl)pyridin-2-yl)-2,6-dichloro-8-fluoro-5-(2-((1-(3-((4-methoxybenzyl)amino)pyridazin-4-yl)ethyl)amino)ethoxy)quinazolin-4(3H)-one (3.00 g, 3.16 mmol, 70.2% yield) as a yellow solid. LC-MS: (ESI, m/z): 947.3 [M+H]⁺

Step 2: 4-(1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine

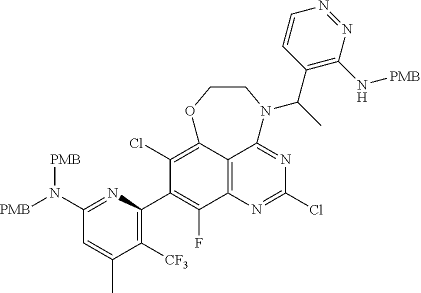

Similar to as described in General Procedure B. A solution of (7R)-7-(6-(bis(4-methoxybenzyl)amino)-4-methyl-3-(trifluoromethyl)pyridin-2-yl)-2,6-dichloro-8-fluoro-5-(2-((1-(3-((4-methoxybenzyl)amino)pyridazin-4-yl)ethyl)amino)ethoxy)quinazolin-4(3H)-one (2.80 g, 2.95 mmol), N,N-diisopropylethylamine (1.90 g, 14.73 mmol) and bis(2-oxo-3-oxazolidinyl)phosphinic chloride (1.10 g, 4.42 mmol) in chloroform (30 mL) was stirred at 70° C. for 2 hours. After completion, the reaction mixture was diluted with dichloromethane, washed with water and the organic layer was combined. The organic layer was dried over anhydrous sodium sulfate and concentrated under vacuum. The residue was purified by flash chromatography on silica gel eluting with dichloromethane/methanol (95:5) to afford 4-(1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine (1.90 g, 2.04 mmol, 69.2% yield) as a brown solid. LC-MS: (ESI, m/z): 929.3 [M+H]⁺

Step 3: 4-(1-(9-((R)-6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyridazin-3-amine

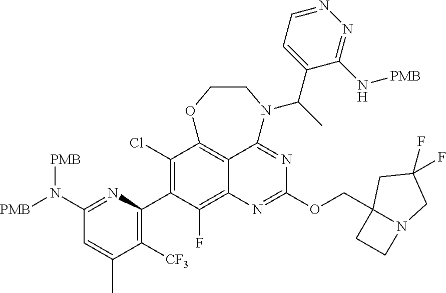

Under nitrogen, sodium bis(trimethylsilyl)amide (2.15 mL, 2.15 mmol, 1 M in tetrahydrofuran) was added to a solution of (3,3-difluoro-1-azabicyclo[3.2.0]heptan-5-yl)methanol (263.2 mg, 1.61 mmol) in tetrahydrofuran (5 mL) and stirred for 20 min at 25° C. Then the reaction mixture was added to a solution of 4-(1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine (500.0 mg, 0.54 mmol) in tetrahydrofuran (5 mL) and stirred at 25° C. for 1 hour. After completion, the reaction was quenched with saturated ammonium chloride solution, diluted with ethyl acetate, washed with water and the organic layer was combined. The organic layer was dried over anhydrous sodium sulfate and concentrated under vacuum. The residue was purified by flash chromatography on silica gel eluting with dichloromethane/methanol (95:5) to afford 4-(1-(9-((R)-6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyridazin-3-amine (160.3 mg, 0.15 mmol, 28.2% yield) as a yellow solid. LC-MS: (ESI, m/z): 1056.4 [M+H]⁺

Step 4: 4-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine & 4-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine & 4-((1S)-1-((9R)-9-(6-amino-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine

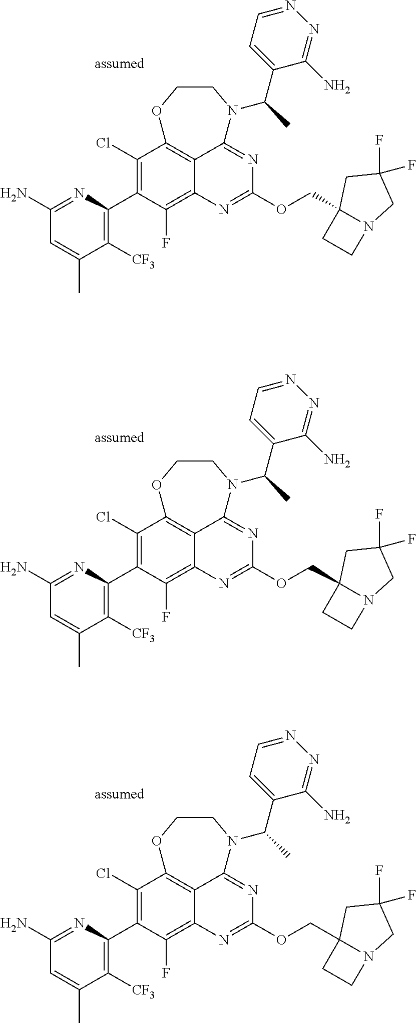

A solution of 4-(1-(9-((R)-6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyridazin-3-amine (150.0 mg, 0.14 mmol) in trifluoroacetic acid (5 mL) and trifluoromethanesulfonic acid (0.5 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm: Mobile Phase A: water (10 mmol/L NH₄HCO₃), Mobile Phase B: acetonitrile; Flow rate: 60 mL/min; Gradient: 24% B to 50% B in 10 min, 50% B; Wave Length: 254/220 nm; RT1(min): 7.98 to afford two diastereoisomers. The diastereoisomer (faster peak) was isolated by Prep-Chiral-HPLC with the following conditions: Column: CHIRALPAK IE, 2*25 cm, 5 μm; Mobile Phase A: Hex:DCM=3:1 (0.5% 2M NH₃-MeOH)-HPLC, Mobile Phase B: IPA-HPLC; Flow rate: 20 mL/min: Gradient: 40% B to 40% B in 26 min: Wave Length: 220/254 nm; RT1(min): 18.69; RT2(min): 23.25; Sample Solvent: EtOH-HPLC to afford 4-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine (3.6 mg, 0.0052 mmol, 3.6% yield) and 4-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine (4.1 mg, 0.0059 mmol, 4.1% yield). The diastereoisomer (slower peak) was submitted as 4-((1S)-1-((9R)-9-(6-amino-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridazin-3-amine (3.6 mg, 0.0052 mmol, 3.6% yield). LC-MS: (ESI, m/z): 696.2 [M+H]⁺

Example 77a: ¹H NMR (300 MHz, Methanol-d₄, ppm) δ 8.55 (d, J=4.8 Hz, 1H), 7.55 (d, J=4.9 Hz, 1H), 6.61 (s, 1H), 6.42 (q, J=6.8 Hz, 1H), 4.68-4.51 (m, 1H), 4.48-4.25 (m, 3H), 3.95-3.77 (m, 2H), 3.74-3.53 (m, 1H), 3.50-3.37 (m, 1H), 3.27-2.95 (m, 2H), 2.84-2.52 (m, 3H), 2.51-2.25 (m, 4H), 1.72 (d, J=6.9 Hz, 3H). LC-MS: (ESI, m/z): 696.2 [M+H]⁺ CHIRALPAK IE-3, 4.6×50 mm, 3 um; detected at 254 nm: (Hex:DCM=3:1)(0.1% DEA):IPA=60:40: Flow rate: 1 mL/min; Retention time: 3.476 min (faster peak)

Example 77b: ¹H NMR (300 MHz, Methanol-d₄, ppm) δ 8.54 (d, J=4.9 Hz, 1H), 7.54 (d, J=5.0 Hz, 1H), 6.61 (s, 1H), 6.42 (q, J=6.9 Hz, 1H), 4.64-4.49 (m, 2H), 4.49-4.28 (m, 2H), 3.91-3.53 (m, 3H), 3.53-3.36 (m, 1H), 3.24-3.05 (m, 2H), 2.73-2.51 (m, 3H), 2.50-2.34 (m, 4H), 1.72 (d, J=6.9 Hz, 3H). LC-MS: (ESI, m/z): 696.2 [M+H]^{+ CHIRALPAK IE-}3, 4.6×50 mm, 3 um; detected at 254 nm; (Hex:DCM=3:1)(0.1% DEA):IPA=60:40; Flow rate: 1 mL/min; Retention time: 4.579 min (slower peak)

Example 77c: ¹H NMR (300 MHz, Methanol-d₄, ppm) δ 8.54 (dd, J=4.9, 2.5 Hz, 1H), 7.55 (dd, J=4.9, 2.3 Hz, 1H), 6.61 (s, 1H), 6.44 (q, J=6.9 Hz, 1H), 4.64-4.36 (m, 4H), 3.94-3.79 (m, 1H), 3.80-3.66 (m, 1H), 3.66-3.51 (m, 1H), 3.49-3.36 (m, 1H), 3.27-3.09 (m, 2H), 2.78-2.49 (m, 3H), 2.50-2.24 (m, 4H), 1.72 (d, J=7.0, 1.3 Hz, 3H). LC-MS: (ESI, W/z): 696.2 [M+H]⁺

Example 78: 6-((R)-4-((R)-1-(2-aminopyridin-3-yl)ethyl)-8-chloro-10-fluoro-2-(2-(1-methyl-1H-imidazol-2-yl)ethoxy)-5,6-dihydro-4H-[1,4]oxazepino[5,6,7-de]quinazolin-9-yl)-4-methyl-5-(trifluoromethyl)pyridin-2-amine

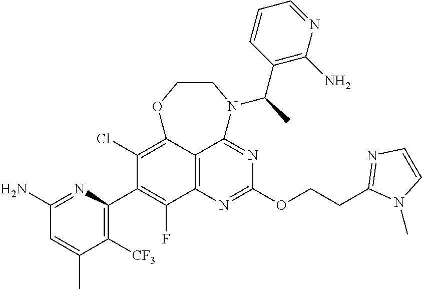

### Synthetic Route

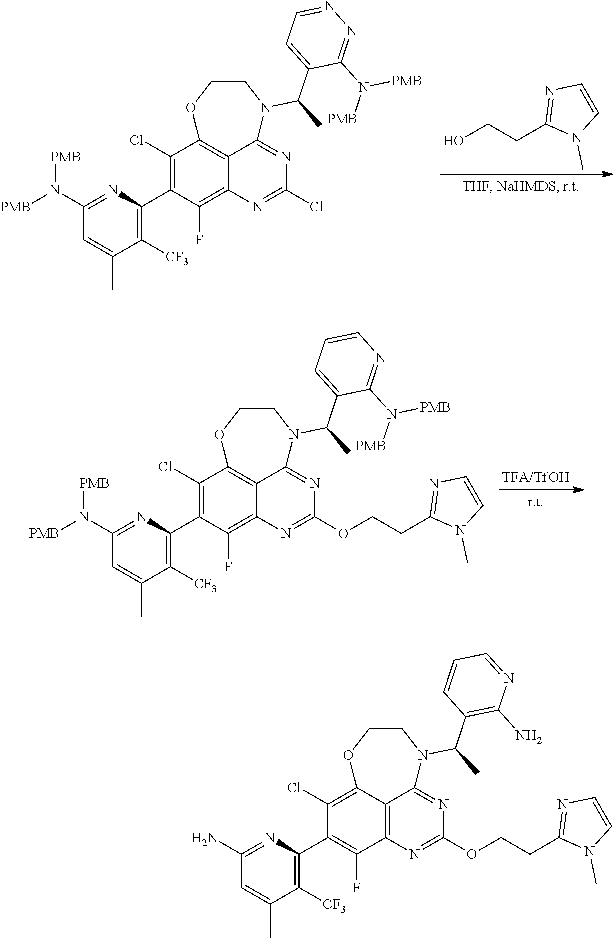

Step 1: (R)-6-(4-((R)-1-(2-(bis(4-methoxybenzyl)amino)pyridin-3-yl)ethyl)-8-chloro-10-fluoro-2-(2-(1-methyl-1H-imidazol-2-yl)ethoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

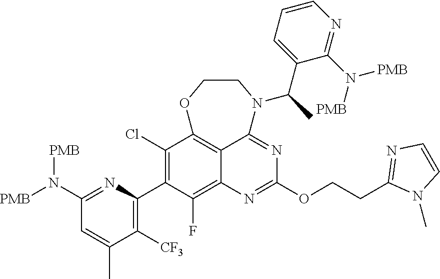

Under nitrogen, to a solution of 2-(1-methyl-1H-imidazol-2-yl)ethan-1-ol (84.2 mg, 0.52 mmol) in tetrahydrofuran (2 mL) was added sodium bis(trimethylsilyl)amide (1.29 mL, 1.29 mmol, 1 M in tetrahydrofuran) at 0° C. and stirred at 25° C. for 1 hour. Under nitrogen, the reaction mixture was added to a solution of (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.0 mg, 0.43 mmol) in tetrahydrofuran (3 mL) at 25° C. and stirred at 25° C. for 5 hours. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with dichloromethane/ethyl acetate (7:3) to afford (R)-6-(4-((R)-1-(2-(bis(4-methoxybenzyl)amino)pyridin-3-yl)ethyl)-8-chloro-10-fluoro-2-(2-(1-methyl-1H-imidazol-2-yl)ethoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (250.3 mg, 0.22 mmol, 51.2% yield). LC-MS: (ESI, m/z): 1138.4 [M+H]⁺

Step 2: 6-((R)-4-((R)-1-(2-aminopyridin-3-yl)ethyl)-8-chloro-10-fluoro-2-(2-(1-methyl-1H-imidazol-2-yl)ethoxy)-5,6-dihydro-4H-[1,4]oxazepino[5,6,7-de]quinazolin-9-yl)-4-methyl-5-(trifluoromethyl)pyridin-2-amine

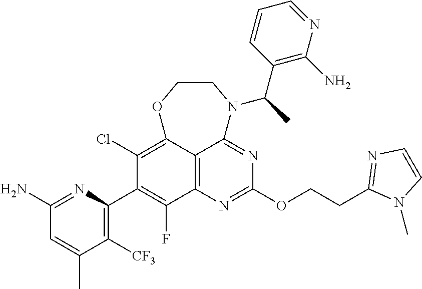

A solution of (R)-6-(4-((R)-1-(2-(bis(4-methoxybenzyl)amino)pyridin-3-yl)ethyl)-8-chloro-10-fluoro-2-(2-(1-methyl-1H-imidazol-2-yl)ethoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (230.0 mg, 0.20 mmol) in trifluoroacetic acid (5 mL) and trifluoromethanesulfonic acid (0.5 mL) was stirred for 1 hour at 25° C. After completion, the reaction mixture was diluted with dichloromethane and adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Shield RP18 OBD Column, 30*150 mm, 5 μm; Mobile Phase A: water (10 mmol/L NH₄HCO₃), Mobile Phase B: acetonitrile; Flow rate: 60 mL/min; Gradient: 34% B to 50% B in 9 min, 50% B; Wave Length: 254/220 nm; RT1(min): 9 to afford 6-((R)-4-((R)-1-(2-aminopyridin-3-yl)ethyl)-8-chloro-10-fluoro-2-(2-(1-methyl-1H-imidazol-2-yl)ethoxy)-5,6-dihydro-4H-[1,4]oxazepino[5,6,7-de]quinazolin-9-yl)-4-methyl-5-(trifluoromethyl)pyridin-2-amine (13.7 mg, 0.02 mmol, 10.3% yield). LC-MS: (ESI, m/z): 658.1 [M+H]⁺

Example 78: ¹H NMR (300 MHz, DMSO-d₆, ppm) δ 7.97 (dd, J=4.8, 1.8 Hz, 1H), 7.63 (dd, J=7.5, 1.7 Hz, 1H), 7.03 (d, J=1.2 Hz, 1H), 6.81 (s, 2H), 6.76 (d, J=1.2 Hz, 1H), 6.67 (q, J=4.8 Hz, 1H), 6.48 (s, 1H), 6.24 (q, J=6.7 Hz, 1H), 5.66 (s, 2H), 4.66 (t, J=7.2 Hz, 2H), 4.52-4.34 (m, 1H), 4.34-4.15 (m, 1H), 3.62 (s, 4H), 3.47-3.36 (m, 1H), 3.13 (t, J=7.2 Hz, 2H), 2.36 (d, J=2.4 Hz, 3H), 1.56 (d, J=6.8 Hz, 3H). LC-MS: (ESI, m/z): 658.1 [M+H]⁺

Example 79: 3-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N⁵-methylpyridine-2,5-diamine

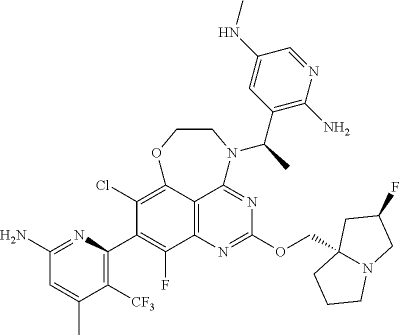

### Synthetic Route

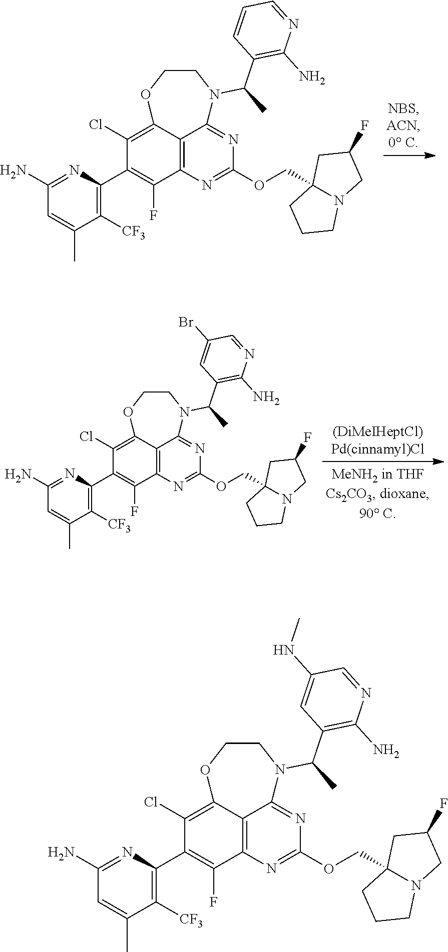

Step 1: 6-((R)-4-((R)-1-(2-amino-5-bromopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

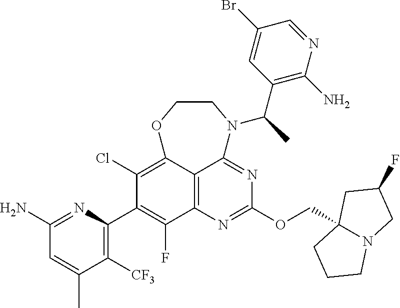

To a solution of 6-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (300.0 mg, 0.43 mmol) in acetonitrile (5 mL) was added N-bromosuccinimide (69.5 mg, 0.39 mmol) and stirred at 0° C. for 0.5 hour. After completion, the resulting solution was quenched by saturated sodium sulfite solution, diluted with ethyl acetate, washed with water and the organic layer was combined. The organic layer was dried over anhydrous sodium sulfate and concentrated under vacuum. The residue was purified by flash chromatography on silica gel eluting with dichloromethane/methanol (1:1) to afford 6-((R)-4-((R)-1-(2-amino-5-bromopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (160.0 mg, 0.19 mmol, 45.5% yield) as a yellow solid. LC-MS: (ESI, m/z): 769.1 [M+H]⁺.

Step 2: 3-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N⁵-methylpyridine-2,5-diamine

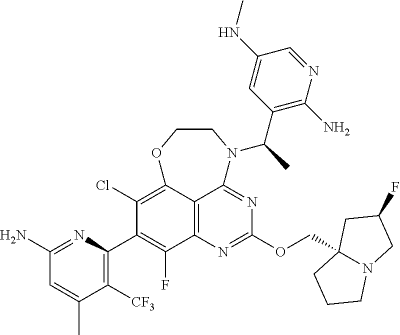

Under nitrogen, a solution of 6-((R)-4-((R)-1-(2-amino-5-bromopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (150.0 mg, 0.19 mmol), cesium carbonate (126.9 mg, 0.39 mmol) and (dimeiheptcl)pd(cinnamyl)cl (22.6 mg, 0.02 mmol) in 1,4-dioxane (5 mL) was stirred at 25° C. for 0.5 hour. Then methylamine (0.26 mL, 0.97 mmol, 1 M in tetrahydrofuran) was added and stirred at 90° C. for 3 hours. After completion, the resulting solution was concentrated under vacuum, diluted with ethyl acetate, washed with water and the organic layer was combined. The organic layer dried over anhydrous sodium sulfate and concentrated under vacuum. The residue was purified by flash chromatography on silica gel eluting with dichloromethane/methanol (7:1) to afford 3-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N5-methylpyridine-2,5-diamine. The crude product was purified by Prep-HPLC with the following conditions: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 30% B to 54% B in 8 min; Wave Length: 254/220 nm; RT1: 8 min to afford 3-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N⁵-methylpyridine-2,5-diamine (11.0 mg, 0.02 mmol, 7.8% yield). LC-MS: (ESI, m/z): 720.2 [M+H]⁺.

Example 79: ¹H NMR (300 MHz, DMSO-d₆, ppm) δ 7.42 (d, J=2.6 Hz, 1H), 7.01 (d, J=2.7 Hz, 1H), 6.82 (s, 2H), 6.48 (s, 1H), 6.24 (q, J=6.6 Hz, 1H), 5.28 (d, J=54.5 Hz, 1H), 5.02 (s, 1H), 4.79 (s, 2H), 4.50-4.34 (m, 1H), 4.32-4.18 (m, 1H), 4.18-3.95 (m, 2H), 3.72-3.55 (m, 1H), 3.20-3.02 (m, 3H), 3.00 (s, 1H), 2.91-2.77 (m, 1H), 2.68 (s, 3H), 2.46-2.29 (m, 3H), 2.28-2.10 (m, 1H), 2.12-1.96 (m, 2H), 1.94-1.70 (m, 3H), 1.54 (d, J=6.8 Hz, 3H). LC-MS: (ESI, m/z): 720.2 [M+H]⁺.

Example 80: 3-((S)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine

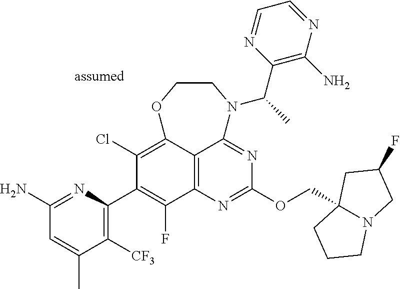

### Synthetic Route

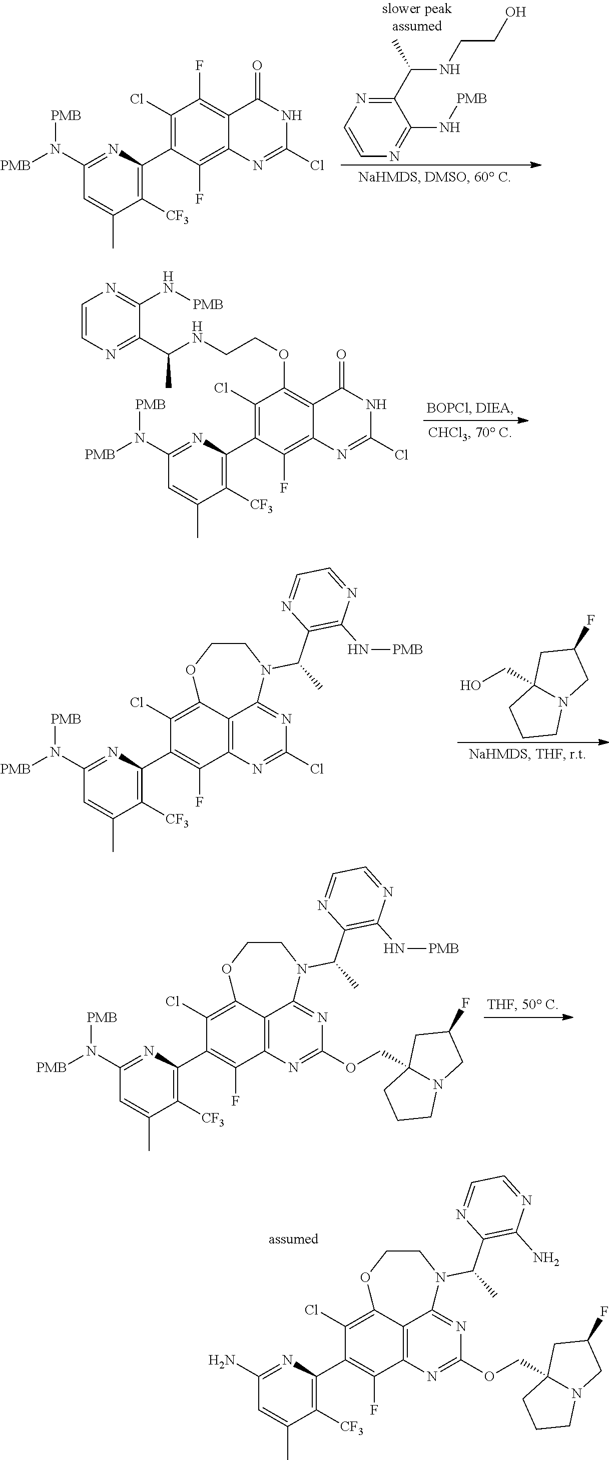

Step 1: 7-((R)-6-(bis(4-methoxybenzyl)amino)-4-methyl-3-(trifluoromethyl)pyridin-2-yl)-2,6-dichloro-8-fluoro-5-(2-(((S)-1-(3-((4-methoxybenzyl)amino)pyrazin-2-yl)ethyl)amino)ethoxy)quinazolin-4(3H)-one

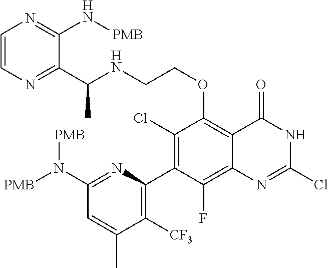

Similar to as described in General Procedure A. Under nitrogen, a solution of (S)-2-((1-(3-((4-methoxybenzyl)amino)pyrazin-2-yl)ethyl)amino)ethan-1-ol (272.6 mg, 0.90 mmol) in dimethyl sulfoxide (5 mL) was added sodium bis(trimethylsilyl)amide (2.25 mL, 2.25 mmol, 1 M in tetrahydrofuran) at 0° C. and stirred at 25° C. for 0.5 hour. Then (R)-7-(6-(bis(4-methoxybenzyl)amino)-4-methyl-3-(trifluoromethyl)pyridin-2-yl)-2,6-dichloro-5,8-difluoroquinazolin-4(3H)-one (500.0 mg, 0.75 mmol) in dimethyl sulfoxide (5 mL) was added and stirred at 60° C. for 1 hour. After completion, the resulting solution was quenched by saturated ammonium chloride solution, diluted with water, extracted with ethyl acetate, washed with water and the organic layer was combined. The organic layer was dried over anhydrous sodium sulfate and concentrated under vacuum to afford 7-((R)-6-(bis(4-methoxybenzyl)amino)-4-methyl-3-(trifluoromethyl)pyridin-2-yl)-2,6-dichloro-8-fluoro-5-(2-(((S)-1-(3-((4-methoxybenzyl)amino)pyrazin-2-yl)ethyl)amino)ethoxy)quinazolin-4(3H)-one (1.0 g, crude) as a black solid. LC-MS: (ESI, m/z): 947.3 [M+H]⁺.

Step 2: 3-((S)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine

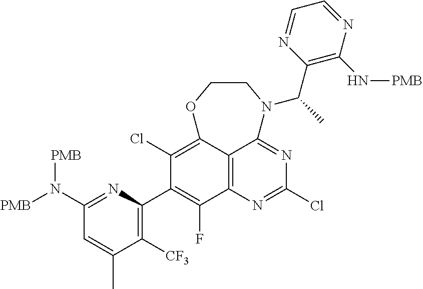

Similar to as described in General Procedure B. A solution of 7-((R)-6-(bis(4-methoxybenzyl)amino)-4-methyl-3-(trifluoromethyl)pyridin-2-yl)-2,6-dichloro-8-fluoro-5-(2-(((S)-1-(3-((4-methoxybenzyl)amino)pyrazin-2-yl)ethyl)amino)ethoxy)quinazolin-4(3H)-one (900.0 mg, crude), bis(2-oxo-3-oxazolidinyl)phosphinic chloride (157.1 mg, 0.62 mmol) and N,N-diisopropylethylamine (612.8 mg, 4.75 mmol) in chloroform (9 mL) was stirred at 70° C. for 1 hour. After completion, the resulting solution was diluted with dichloromethane, washed with water and the organic layer was combined. The organic layer was dried over anhydrous sodium sulfate and concentrated under vacuum. The residue was purified by flash chromatography on silica gel eluting with dichloromethane/methanol (8:1) to afford 3-((S)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (450.0 mg, 0.38 mmol, 81.5% yield) as a yellow solid. LC-MS: (ESI, m/z): 929.3 [M+H]⁺.

Step 3: 3-((S)-1-((R)-9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine

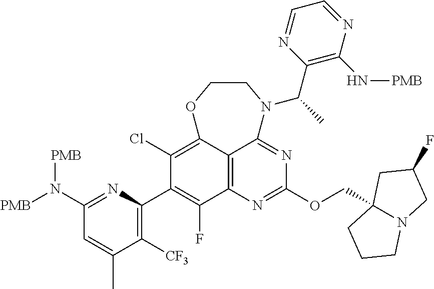

Under nitrogen, sodium bis(trimethylsilyl)amide (1.94 mL, 1.94 mmol, 1 M in tetrahydrofuran) was added to the solution of ((2R,7aS)-2-fluorotetrahydro-1H-pyrrolizin-7a(5H)-yl)methanol (269.6 mg, 1.69 mmol) in tetrahydrofuran (5 mL) and stirred at 25° C. for 0.5 hour. Then 3-((S)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (450.0 mg, 0.38 mmol) in tetrahydrofuran (5 mL) was added and stirred at 25° C. for 1 hour. After completion, the resulting solution was quenched by saturated ammonium chloride solution, diluted with ethyl acetate, washed with water and the organic layer was combined. The organic layer was dried over anhydrous sodium sulfate and concentrated under vacuum. The residue was purified by flash chromatography on silica gel eluting with dichloromethane/methanol (8:1) to afford 3-((S)-1-((R)-9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine (140.0 mg, 0.09 mmol, 19.2% yield) as a yellow solid. LC-MS: (ESI, m/z): 1052.4 [M+H]⁺.

Step 4: 3-((S)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine

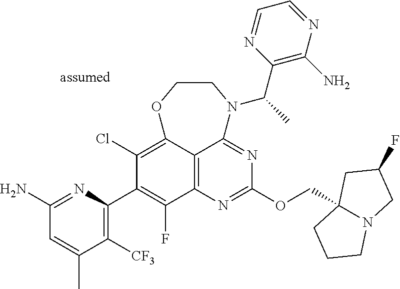

A solution of 3-((S)-1-((R)-9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine (140.0 mg, 0.09 mmol) in 2,2,2-trifluoroacetic acid (2 mL) was stirred at 50° C. for 36 hours. After completion, the resulting solution was diluted with dichloromethane and adjusted to pH >7 with sodium bicarbonate saturated solution, extracted with dichloromethane and the organic layer was combined.

The organic layer was dried over anhydrous sodium sulfate and concentrated under vacuum to afford product. The crude product was purified by Prep-HPLC with the following conditions: XSelect CSH Fluoro Phenyl, 30*150 mm, 5 μm; Mobile Phase A: water (10 mmol/L NH₄HCO₃), Mobile Phase B: acetonitrile; Flow rate: 60 mL/min; Gradient: 44% B to 69% B in 9 min; Wave Length: 254/220 nm; RT1: 7.63 min to afford 3-((S)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine (19.4 mg, 0.03 mmol, 21.1% yield). LC-MS: (ESI, m/z): 692.2 [M+H]⁺.

Example 80: ¹H NMR (300 MHz, DMSO-d₆, ppm)) δ 7.94 (d, J=2.7 Hz, 1H), 7.77 (d, J=2.7 Hz, 1H), 6.82 (s, 2H), 6.49 (d, J=6.5 Hz, 3H), 6.26 (q, J=6.8 Hz, 1H), 5.29 (d, J=54.6 Hz, 1H), 4.72-4.55 (m, 1H), 4.45-4.33 (m, 1H), 4.13 (d, J=10.4 Hz, 1H), 4.02-3.83 (m, 2H), 3.72-3.57 (m, 1H), 3.21-2.93 (m, 3H), 2.90-2.72 (m, 1H), 2.37 (d, J=2.3 Hz, 3H), 2.13 (d, J=6.3 Hz, 1H), 2.08-1.90 (m, 2H), 1.90-1.68 (m, 3H), 1.60 (d, J=6.8 Hz, 3H). LC-MS: (ESI, m/z): 692.2 [M+H]⁺.

Example 81: 5-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrimidin-4-amine

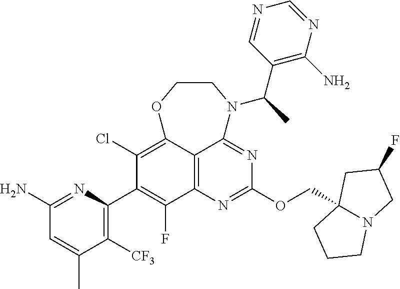

### Synthetic Route

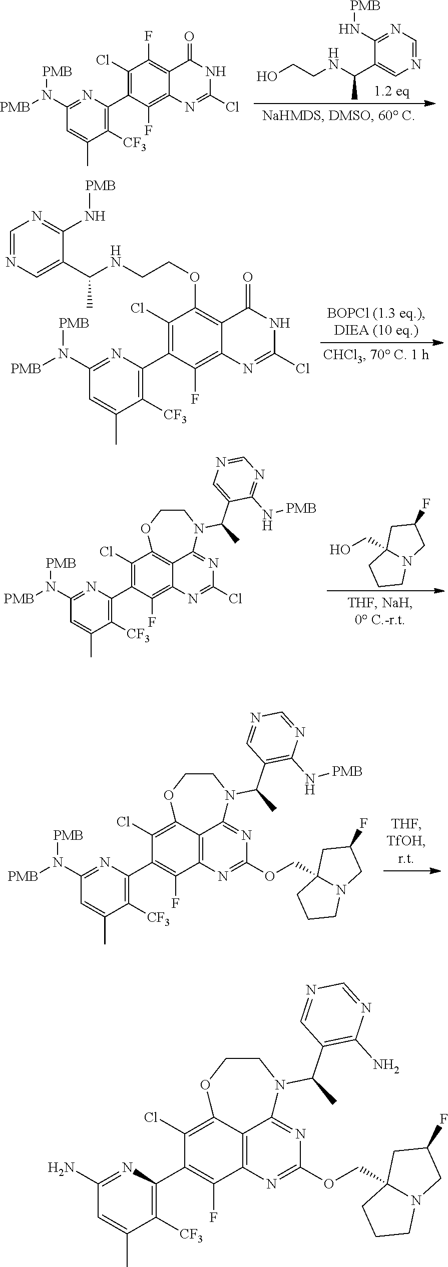

Step 1: 7-(6-(bis(4-methoxybenzyl)amino)-4-methyl-3-(trifluoromethyl)pyridin-2-yl)-2,6-dichloro-8-fluoro-5-(2-(((R)-1-(4-((4-methoxybenzyl)amino)pyrimidin-5-yl)ethyl)amino)ethoxy)quinazolin-4(3H)-one

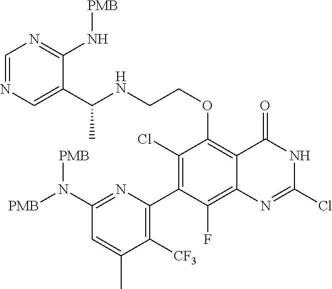

Similar to as described in General Procedure A. Under nitrogen, to a mixture of 2-[[(1R)-1-[4-[(4-methoxyphenyl)methylamino]pyrimidin-5-yl]ethyl]amino]ethanol (11.49 g, 38.02 mmol) in dimethyl sulfoxide (230 mL) was added sodium bis(trimethylsilyl)amide (103.69 mL, 103.69 mmol, 1M in tetrahydrofuran), the mixture was stirred for 10 minutes at room temperature. The mixture was transferred into a solution of 7-[6-[bis[(4-methoxyphenyl)methyl]amino]-4-methyl-3-(trifluoromethyl)-2-pyridyl]-2,6-dichloro-5,8-difluoro-3H-quinazolin-4-one (23.00 g, 34.56 mmol) in dimethyl sulfoxide (230 mL) and stirred at 60° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product was used in the next step without purification. LCMS (ESI, m/z): 947.3 [M+H]⁺.

Step 2: 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine

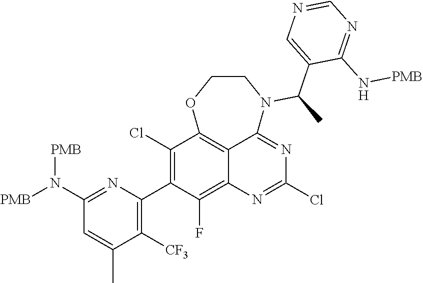

Similar to as described in General Procedure B. A mixture of 7-(6-(bis(4-methoxybenzyl)amino)-4-methyl-3-(trifluoromethyl)pyridin-2-yl)-2,6-dichloro-8-fluoro-5-(2-(((R)-1-(4-((4-methoxybenzyl)amino)pyrimidin-5-yl)ethyl)amino)ethoxy)quinazolin-4(3H)-one (34.00 g, 35.87 mmol), bis(2-oxo-3-oxazolidinyl)phosphinicchloride (13.60 g, 53.42 mmol) and N,N-diisopropylethylamine (23.12 g, 178.89 mmol) in chloroform (350 mL) was stirred at 70° C. for 1 hour. After completion, the reaction was diluted with water and extracted with dichloromethane. The organic layers were combined, washed with brine, dried over anhydrous sodium sulfate and concentrated. The residue was purified by flash chromatography on silica gel eluting with dichloromethane/methanol (95:5) to afford 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (25 g, 18.28 mmol, 51% yield) as a yellow solid.

LCMS (ESI, m/z): 929.3 [M+H]⁺.

Step 3: 5-((R)-1-((R)-9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyrimidin-4-amine

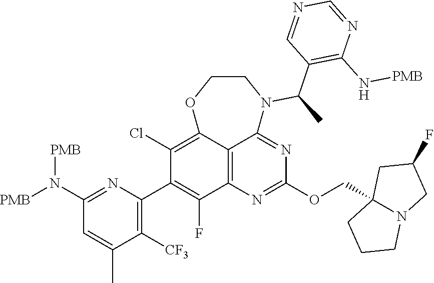

To a mixture of [(2R,8S)-2-fluoro-1,2,3,5,6,7-hexahydropyrrolizin-8-yl]methanol (2.05 g, 12.91 mmol) in tetrahydrofuran (40 mL) was added sodium hydride (688.3 mg, 17.21 mmol, 60% dispersion in mineral oil), the mixture was stirred for 10 minutes at 0° C. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (4.00 g, 4.30 mmol) in tetrahydrofuran (5 mL) was added, the mixture was stirred for 1 hour at room temperature. The reaction was quenched with 1M hydrochloric acid. The resulting solution was diluted with water and extracted with ethyl acetate. The organic layers were combined, washed with brine, dried over anhydrous sodium sulfate and concentrated. The crude product was used in the next step without purification. LCMS (ESI, m/z): 1052.4 [M+H]⁺.

Step 4: 5-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrimidin-4-amine

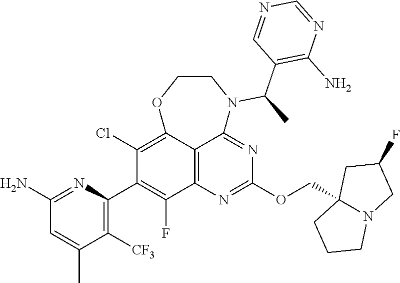

A mixture of 5-((R)-1-((R)-9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyrimidin-4-amine (5.00 g, 4.75 mmol) in trifluoroacetic acid (150 mL) and trifuoromethanesulfonic acid (15 mL) was stirred at room temperature for 16 hours. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The resulting residue was purified by reverse phase chromatography (acetonitrile/0.1% NH₄HCO₃ in water) to afford crude product. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 28% B to 48% B in 9 min, 48% B; Wave Length: 254/220 nm; RT1(min): 8.38 to afford 5-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrimidin-4-amine (494.4 mg, 0.7144 mmol, 15% yield). LCMS (ESI, m/z): 692.2 [M+H]⁺.

Example 81: ¹H NMR (300 MHz, DMSO-d₆, ppm) δ 8.37 (s, 1H), 8.28 (s, 1H), 6.80 (s, 4H), 6.46 (s, 1H), 6.15 (q, J=7.0 Hz, 1H), 5.26 (d, J=54.7 Hz, 1H), 4.47 (dd, J=12.1, 6.1 Hz, 1H), 4.31 (dd, J=12.0, 6.6 Hz, 1H), 4.05 (dd, 2H), 3.71 (dd, J=15.6, 6.8 Hz, 1H), 3.46 (dd, J=15.4, 6.2 Hz, 1H), 3.18-3.02 (m, 2H), 2.98 (s, 1H), 2.90-2.73 (m, 1H), 2.35 (d, J=2.2 Hz, 3H), 2.19-2.09 (m, 1H), 2.09-1.90 (m, 2H), 1.88-1.67 (m, 3H), 1.59 (d, J=6.8 Hz, 3H).

Example 82a & 82b: 5-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrimidin-4-amine & 5-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrimidin-4-amine

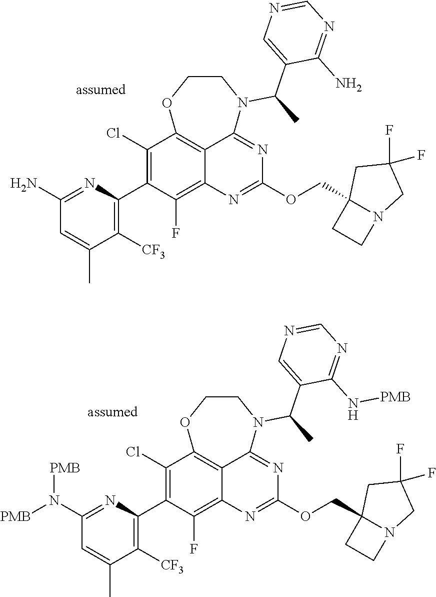

### Synthetic Route

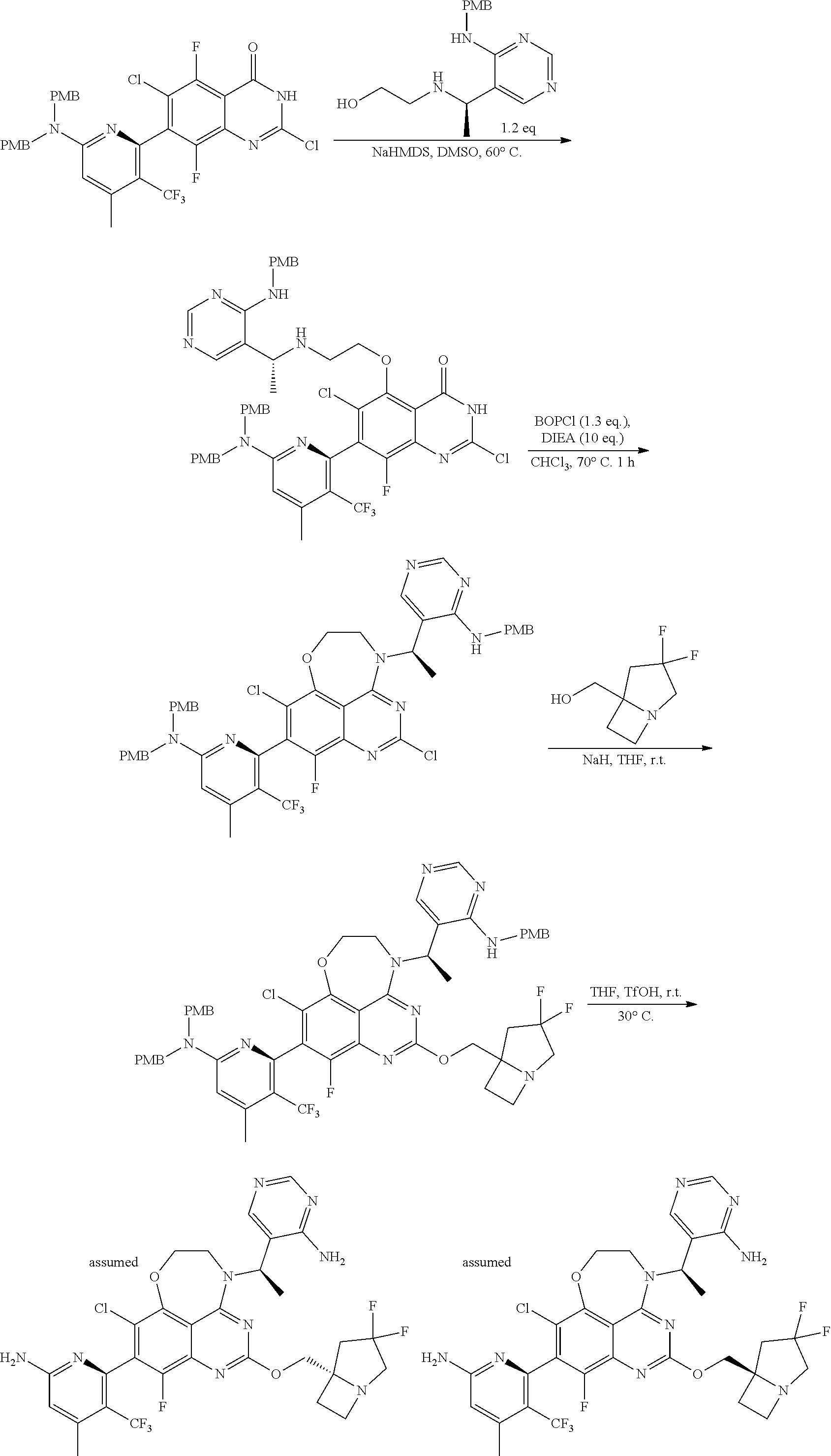

Step 1: (R)-7-(6-(bis(4-methoxybenzyl)amino)-4-methyl-3-(trifluoromethyl)pyridin-2-yl)-2,6-dichloro-8-fluoro-5-(2-(((R)-1-(4-((4-methoxybenzyl)amino)pyrimidin-5-yl)ethyl)amino)ethoxy)quinazolin-4(3H)-one

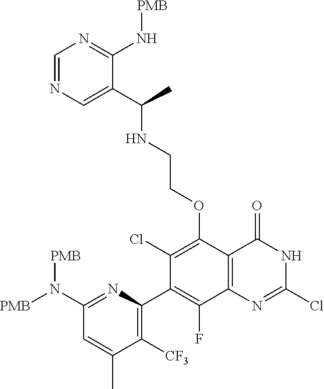

Similar to as described in General Procedure A. Under nitrogen, to a mixture of 2-[[(1R)-1-[4-[(4-methoxyphenyl)methylamino]pyrimidin-5-yl]ethyl]amino]ethanol (550.0 mg, 1.82 mmol) in Dimethyl sulfoxide (11 mL) was added sodium bis(trimethylsilyl)amide (4.96 mL, 4.96 mmol, 1M in tetrahydrofuran), the mixture was stirred for 10 min at room temperature. The mixture was added into a solution of 7-[6-[bis[(4-methoxyphenyl)methyl]amino]-4-methyl-3-(trifluoromethyl)-2-pyridyl]-2,6-dichloro-5,8-difluoro-3H-quinazolin-4-one (1.10 g, 1.65 mmol) in Dimethyl sulfoxide (11 mL) and stirred at 60° C. for 1 h. After completion, the reaction was quenched with saturated ammonium chloride solution. The resulting solution was diluted with water and extracted with ethyl acetate. The organic layers were washed with brine, dried over anhydrous Sodium sulfate and concentrated. The crude was used directly without further purification. LCMS (ESI, m/z): 947.3 [M+H]⁺.

Step 2: 5-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine

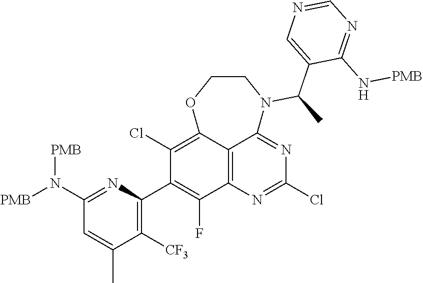

Similar to as described in General Procedure B. A mixture of (R)-7-(6-(bis(4-methoxybenzyl)amino)-4-methyl-3-(trifluoromethyl)pyridin-2-yl)-2,6-dichloro-8-fluoro-5-(2-(((R)-1-(4-((4-methoxybenzyl)amino)pyrimidin-5-yl)ethyl)amino)ethoxy)quinazolin-4(3H)-one (2.30 g, 2.43 mmol), bis(2-oxo-3-oxazolidinyl)phosphinicchloride (920.0 mg, 3.61 mmol) and N,N-diisopropylethylamine (1.56 g, 12.1 mmol) in chloroform (30 mL) was stirred at 70° C. for 1 h. The resulting solution was diluted with water and extracted with Ethyl acetate. The organic layers were washed with brine, dried over anhydrous Sodium sulfate and concentrated. The residue was purified by flash chromatography on silica gel eluting with dichloromethane/ethyl acetate (63:37) to afford 5-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (1.00 g, 1.07 mmol, 44.3% yield) as a yellow solid.

LCMS (ESI, m/z): 929.3 [M+H]⁺.

Step 3: 5-((1R)-1-((9R)-9-(6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine

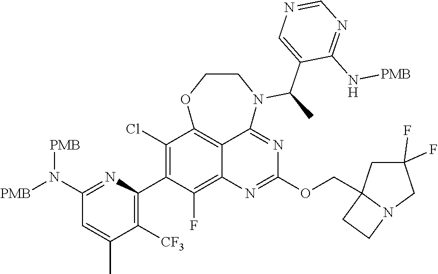

Under nitrogen, to a solution of (3,3-difluoro-1-azabicyclo[3.2.0]heptan-5-yl)methanol (526.4 mg, 3.23 mmol) in tetrahydrofuran (10 mL) was added sodium hydride (172.0 mg, 4.30 mmol, 60% dispersion in mineral oil), the mixture was stirred at 25° C. for 0.5 hour. Then 5-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (1.00 g, 1.08 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product was used in the next step without purification. LC-MS: (ESI, m/z): 1056.4 [M+H]⁺.

Step 4: 5-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrimidin-4-amine & 5-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrimidin-4-amine

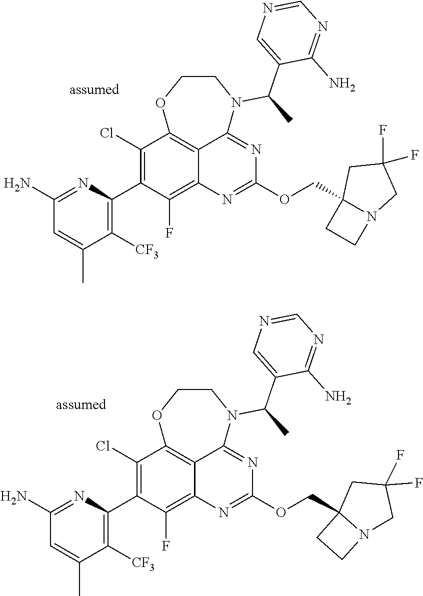

A solution of 5-((1R)-1-((9R)-9-(6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine (1.1 g, crude) in trifluoroacetic acid (20 mL) and trifuoromethanesulfonic acid (2 mL) was stirred at room temperature for 40 hours. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The resulting residue was purified by reverse phase chromatography (acetonitrile/0.1% NH₄HCOs in water) to afford crude product. The crude product was purified by Prep-HPLC with the following conditions: XSelect CSH Fluoro Phenyl, 30*150 mm, 5 μm; Mobile Phase A: water (0.1% FA), Mobile Phase B: acetonitrile; Flow rate: 60 mL/min; Gradient: 5% B to 13% B in 11 min; 254/220 nm; RT1: 9.6 min to afford product. The product was further purified by Prep-CHIRAL-HPLC with the following conditions: Column: CHIRALPAK ID, 2*25 cm, 5 μm; Mobile Phase A: Hex:DCM=3:1(0.5% 2M NH3-MeOH)-HPLC, Mobile Phase B: IPA-HPLC; Flow rate: 20 mL/min; Gradient: 20% B to 20% B in 25 min; Wave Length: 220/254 nm; RT1(min): 7.996; RT2(min): 17.538; Sample Solvent: EtOH-HPLC; Injection Volume: 0.6 mL; Number Of Runs: 6 to afford 5-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrimidin-4-amine (34.6 mg, 0.05 mmol, 4.8% yield) and 5-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrimidin-4-amine (36.3 mg, 0.05 mmol, 5% yield). The stereo chemistry of title compounds was arbitrarily assigned.

Example 82a: ¹H NMR (300 MHz, DMSO-d₆, ppm) δ 8.39 (s, 1H), 8.31 (s, 1H), 6.81 (s, 4H), 6.48 (s, 1H), 6.18 (d, J=6.9 Hz, 1H), 4.56-4.43 (m, 1H), 4.39-4.25 (m, 3H), 3.73 (dd, J=15.2, 7.0 Hz, 1H), 3.61-3.44 (m, 2H), 3.27-3.04 (m, 3H), 2.88-2.58 (m, 1H), 2.48-2.20 (m, 6H), 1.61 (d, J=6.9 Hz, 3H). LC-MS: (ESI, m/z): 696.1 [M+H]⁺, Flow: 1.0 mL/min Chiral HPLC: Column: CHIRALPAK ID-3, 4.6*50 mm, 3 um; detected at 254 nm; (Hex:DCM=3:1)(0.1% DEA):IPA=80:20; Flow rate: 1 mL/min; Retention time: 1.889 min (faster peak)

Example 82b: ¹H NMR (300 MHz, DMSO-d₆, ppm) δ 8.39 (s, 1H), 8.30 (s, 1H), 6.81 (s, 2H), 6.75 (s, 2H), 6.48 (s, 1H), 6.17 (q, J=6.9 Hz, 1H), 4.58-4.42 (m, 1H), 4.44-4.25 (m, 3H), 3.73 (dd, J=15.8, 6.6 Hz, 1H), 3.60-3.43 (m, 2H), 3.27-3.02 (m, 3H), 2.73-2.54 (m, 1H), 2.48-2.23 (m, 6H), 1.62 (d, J=6.8 Hz, 3H). LC-MS: (ESI, m/z): 696.1 [M+H]⁺, Flow: 1.0 mL/min Chiral HPLC: Column: CHIRALPAK ID-3, 4.6*50 mm, 3 um; detected at 254 nm; (Hex:DCM=3:1)(0.1% DEA):IPA=80:20; Flow rate: 1 mL/min; Retention time: 2.887 min (slower peak)

Example 83a & 83b: 5-((R)-1-((R)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyrimidin-4-amine

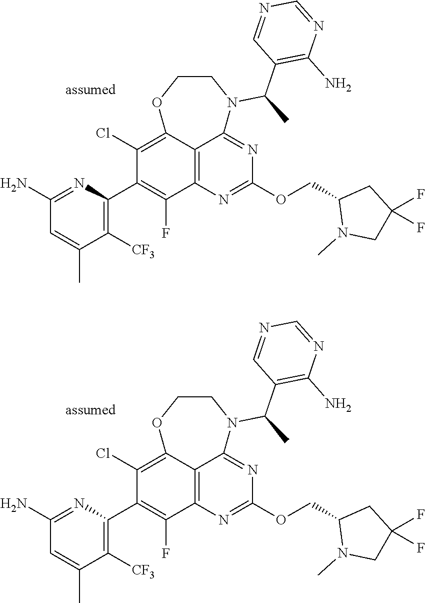

### Synthetic Route

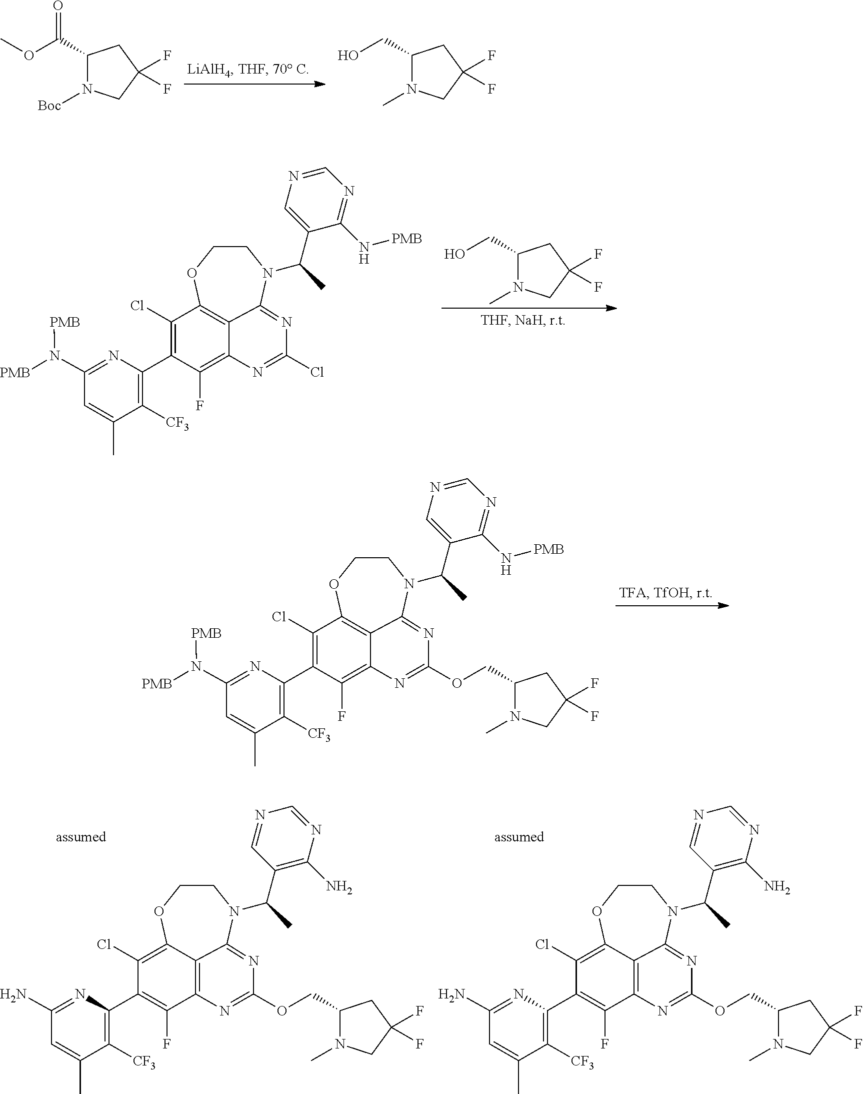

Step 1: (S)-(4,4-difluoro-1-methylpyrrolidin-2-yl)methanol

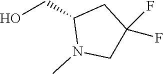

To a solution of 1-(tert-butyl) 2-methyl (S)-4,4-difluoropyrrolidine-1,2-dicarboxylate (5.00 g, 18.85 mmol) in tetrahydrofuran (50 mL) was added lithium aluminium hydride (2.15 g, 56.68 mmol), the mixture was stirred at 70° C. for 1 hour. After completion, the reaction was quenched with sodium sulfate decahydrate. The solids were filtered out. After filtration, the filtrate was concentrated under reduced pressure. The crude product was used in the next step without purification. LC-MS: (ESI, m/z): 152.1 [M+H]⁺

Step 2: 5-((1R)-1-(9-(6-(bis(4-methoxybenzyl)amino)-4-methyl-3-(trifluoromethyl)pyridin-2-yl)-8-chloro-2-(((S)-4,4-<difluoro-1-methylpyrrolidin-2-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine

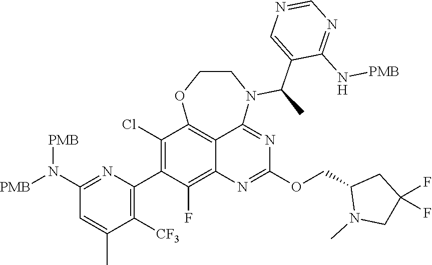

To a solution of [(2S)-4,4-difluoro-1-methyl-pyrrolidin-2-yl]methanol (650.0 mg, 4.3 mmol) in tetrahydrofuran (10 mL) was added sodium hydride (215.0 mg, 5.38 mmol, 60% dispersion in mineral oil), the mixture was stirred at 0° C. for 10 min. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (1.00 g, 1.08 mmol) was added and stirred at 25° C. for 1 hour. After completion, the resulting solution was quenched with saturated ammonium chloride solution and extracted with ethyl acetate. Then the organic layers were combined, washed with brine, dried over anhydrous sodium sulfate and concentrated under vacuum. The crude product was used in the next step without purification. LC-MS: (ESI, m/z): 1044.4 [M+H]⁺

Step 3: 5-((R)-1-((R)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyrimidin-4-amine

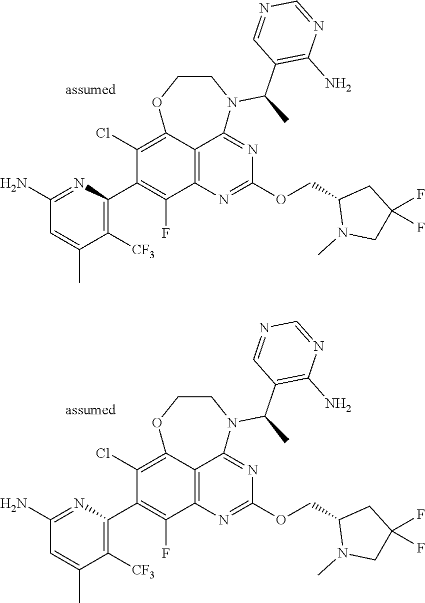

A solution of 5-((1R)-1-(9-(6-(bis(4-methoxybenzyl)amino)-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine (1.60 g, 1.53 mmol) in trifluoroacetic acid (16 mL) and trifuoromethanesulfonic acid (1.6 mL) was stirred at room temperature for 16 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min: Gradient: 31% B to 56% B in 9 min, 56% B; Wave Length: 254/220 nm; RT1(min): 8.7 to afford 5-((R)-1-((R)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyrimidin-4-amine (60.6 mg, 0.09 mmol, 5.7% yield) and 5-((R)-1-((S)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyrimidin-4-amine (64.8 mg, 0.09 mmol, 5.8% yield).

Example 83a: LC-MS: (ESI, m/z): 684.2 [M+H]⁺, ¹H NMR (300 MHz, DMSO-d₆, ppm) δ 8.39 (s, 1H), 8.30 (s, 1H), 6.81 (s, 2H), 6.75 (s, 2H), 6.48 (s, 1H), 6.15 (q, J=6.8 Hz, 1H), 4.57-4.40 (m, 2H), 4.40-4.28 (m, 2H), 3.72 (dd, J=15.8, 6.1 Hz, 1H), 3.55-3.35 (m, 2H), 3.07-2.88 (m, 1H), 2.78-2.54 (m, 2H), 2.43-2.31 (m, 6H), 2.30-2.10 (m, 1H), 1.61 (d, J=6.8 Hz, 3H).

Example 83b: LC-MS: (ESI, m/z): 684.1 [M+H]⁺, ¹H NMR (300 MHz, DMSO-d₆, ppm) δ 8.38 (s, 1H), 8.30 (s, 1H), 6.81 (s, 4H), 6.48 (s, 1H), 6.18 (q, J=6.8 Hz, 1H), 4.63-4.50 (m, 1H), 4.46-4.29 (m, 3H), 3.79 (dd, J=15.6, 6.3 Hz, 1H), 3.60-3.33 (m, 2H), 3.07-2.86 (m, 1H), 2.76-2.54 (m, 2H), 2.42-2.32 (m, 6H), 2.30-2.09 (m, 1H), 1.63 (d, J=6.9 Hz, 3H).

Example 84a & 84b: 5-((R)-1-((R)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyrimidin-4-amine

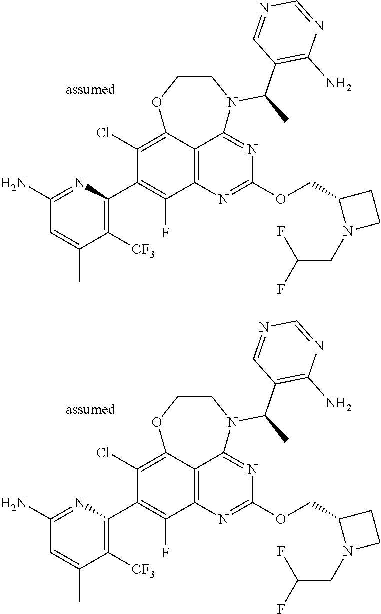

### Synthetic Route

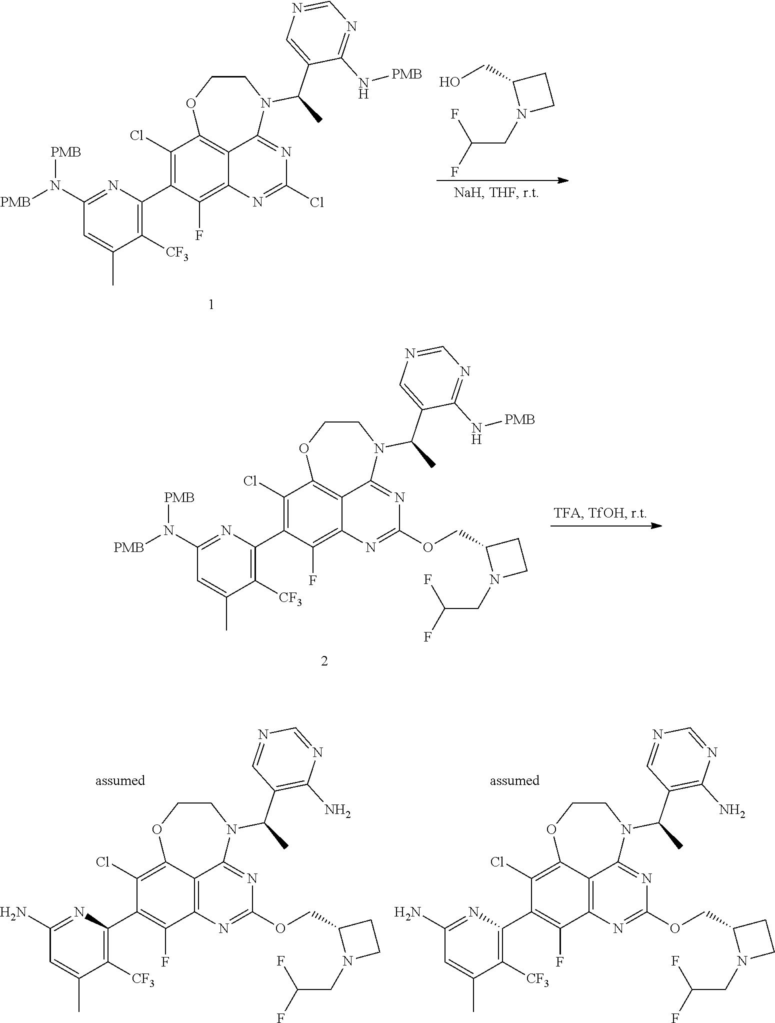

Step 1: 5-((1R)-1-(9-(6-(bis(4-methoxybenzyl)amino)-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine

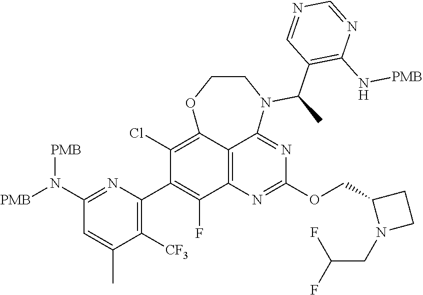

To a solution of [(2S)-1-(2,2-difluoroethyl)azetidin-2-yl]methanol (366.9 mg, 2.43 mmol) in tetrahydrofuran (8 mL) was added sodium hydride (87.4 mg, 3.64 mmol, 60% dispersion in mineral oil), the mixture was stirred at 0° C. for 5 mins. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (566.0 mg, 0.61 mmol) was added and stirred at 25° C. for 10 hours. After completion, the solvent was quenched with dilute hydrochloric acid and extracted with ethyl acetate. The organic layers were combined, dried over anhydrous sodium sulfate and concentrated under vacuum. The crude product was directly used in the next step without purification. LC-MS: (ESI, m/z): 1044.3[M+H]⁺

Step 2: 5-((R)-1-((R)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyrimidin-4-amine

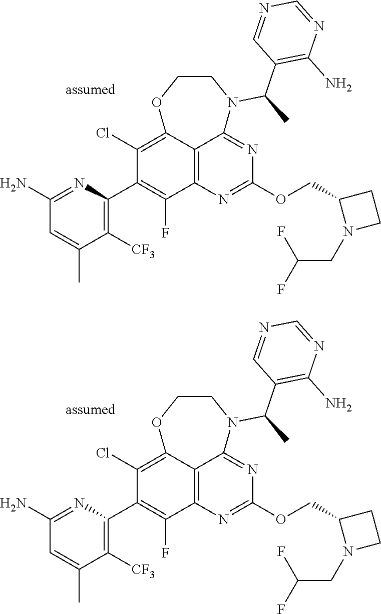

A solution of 5-((1R)-1-(9-(6-(bis(4-methoxybenzyl)amino)-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine (556.0 mg, 0.53 mmol) in trifluoroacetic acid (7 mL) and trifuoromethanesulfonic acid (0.7 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (90:10) to afford crude product. The crude product was purified by Prep-HPLC with the following conditions: Column: X select CSH C18 OBD Column 30*150 mm 5 μm, n; Mobile Phase A: Water (0.1% FA), Mobile Phase B: ACN; Flow rate: 60 mL/min; Gradient: 8% B to 27% B in 10 min, 27% B; Wave Length: 254/220 nm; RT1(min): 9, RT2(min): 10 to afford 5-((R)-1-((R)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyrimidin-4-amine (11.1 mg, 0.02 mmol, 3% yield) and 5-((R)-1-((S)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyrimidin-4-amine (10.5 mg, 0.02 mmol, 2.9% yield).

Example 84a: ¹H NMR (300 MHz, DMSO-d₆, ppm) δ 8.39 (s, 1H), 8.30 (s, 1H), 6.81 (s, 2H), 6.74 (s, 2H), 6.48 (s, 1H), 6.16-5.75 (m, 2H), 4.53-4.26 (m, 4H), 3.77-3.55 (m, 2H), 3.47 (dd, J=15.0, 6.2 Hz, 2H), 3.17-2.93 (m, 2H), 2.87-2.65 (m, 1H), 2.37 (s, 3H), 2.13-1.97 (m, 2H), 1.60 (d, J=6.9 Hz, 3H). LC-MS: (ESI, m/z): 684.1 [M+H]⁺

Example 84b: ¹H NMR (300 MHz, DMSO-d₆, ppm) δ 8.38 (s, 1H), 8.29 (s, 1H), 6.81 (s, 4H), 6.48 (s, 1H), 6.20-5.77 (m, 2H), 4.55 (dd, J=12.3, 6.6 Hz, 1H), 4.44-4.25 (m, 3H), 3.78 (dd, J=15.6, 6.3 Hz, 1H), 3.67-3.57 (m, 1H), 3.53-3.40 (m, 2H), 3.17-2.93 (m, 2H), 2.87-2.65 (m, 1H), 2.37 (s, 3H), 2.13-1.96 (m, 2H), 1.62 (d, J=6.8 Hz, 3H). LC-MS: (ESI, m/z): 684.1 [M+H]⁺

Example 85: 4-((R)-1-((R)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyridazin-3-amine

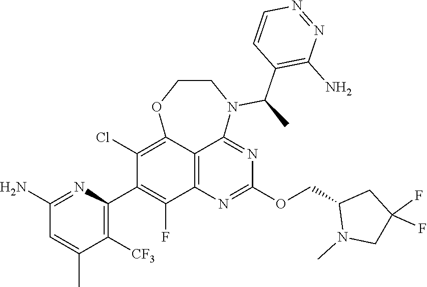

### Synthetic Route

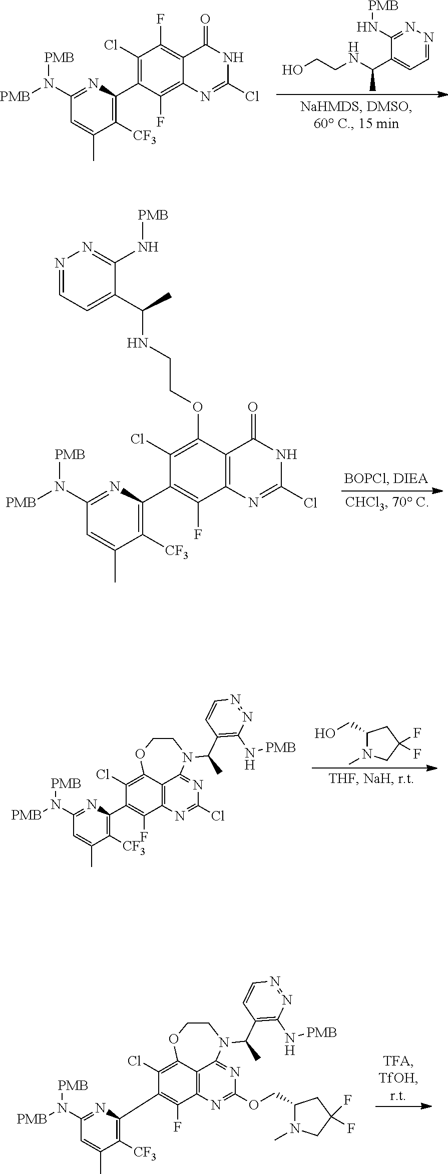

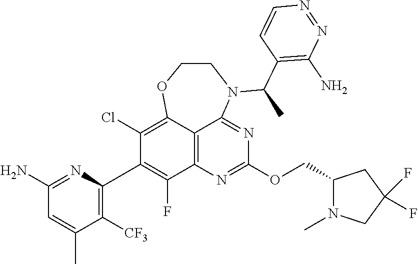

Step 1: (R)-7-(6-(bis(4-methoxybenzyl)amino)-4-methyl-3-(trifluoromethyl)pyridin-2-yl)-2,6-dichloro-8-fluoro-5-(2-(((R)-1-(3-((4-methoxybenzyl)amino)pyridazin-4-yl)ethyl)amino)ethoxy)quinazolin-4(3H)-one

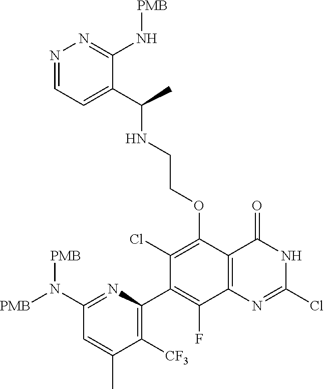

Similar to as described in General Procedure A. Under nitrogen, to a mixture of 2-[[(1R)-1-[3-[(4-methoxyphenyl)methylamino]pyridazin-4-yl]ethyl]amino]ethanol (500.0 mg, 1.65 mmol) in dimethyl sulfoxide (10 mL) was added sodium bis(trimethylsilyl)amide (4.51 mL, 4.51 mmol, 1M in tetrahydrofuran), the mixture was stirred for 10 min at room temperature. The mixture was transferred into a mixture of 7-[6-[bis[(4-methoxyphenyl)methyl]amino]-4-methyl-3-(trifluoromethyl)-2-pyridyl]-2,6-dichloro-5,8-difluoro-3H-quinazolin-4-one (1.00 g, 1.50 mmol) in dimethyl sulfoxide (10 mL) and stirred at 60° C. for 15 min. The reaction was quenched with saturated ammonium chloride solution. The resulting solution was diluted with water and extracted with ethyl acetate. The organic layers were washed with brine, dried over anhydrous sodium sulfate and concentrated. The crude product was used in the next step without purification. LCMS (ESI, m/z): 947.3 [M+H]⁺.

Step 2: 4-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine

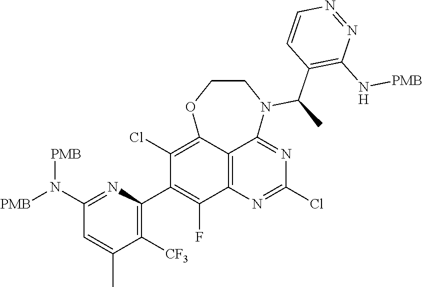

Similar to as described in General Procedure B. A mixture of (R)-7-(6-(bis(4-methoxybenzyl)amino)-4-methyl-3-(trifluoromethyl)pyridin-2-yl)-2,6-dichloro-8-fluoro-5-(2-(((R)-1-(3-((4-methoxybenzyl)amino)pyridazin-4-yl)ethyl)amino)ethoxy)quinazolin-4(3H)-one (1.40 g, 1.48 mmol), bis(2-oxo-3-oxazolidinyl)phosphinicchloride (560.0 mg, 2.20 mmol) and N,N-diisopropylethylamine (952.0 mg, 7.37 mmol) and in chloroform (20 mL) was stirred at 70° C. for 1 hour. The resulting solution was diluted with water and extracted with dichloromethane. The organic layers were washed with brine, dried over anhydrous sodium sulfate and concentrated. The residue was purified by flash chromatography on silica gel eluting with dichloromethane/methanol (95:5) to afford 4-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine (750 mg, 0.80 mmol, 54.6% yield) as a yellow solid.

LCMS (ESI, m/z): 929.3 [M+H]⁺.

Step 3: 4-((R)-1-((R)-9-(6-(bis(4-methoxybenzyl)amino)-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)-N-(4-methoxybenzyl)pyridazin-3-amine

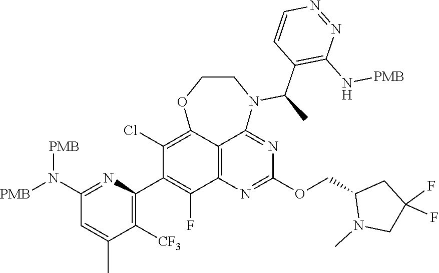

To a mixture of [(2S)-4,4-difluoro-1-methyl-pyrrolidin-2-yl]methanol (192.0 mg, 1.27 mmol) in tetrahydrofuran (10 mL) was added sodium hydride (80.0 mg, 2.0 mmol, 60% dispersion in mineral oil), the mixture was stirred for 10 min at 0° C. Then 4-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine (400.0 mg, 0.43 mmol) in tetrahydrofuran (1 mL) was added, the mixture was stirred for 1 hour at room temperature. The reaction was quenched with saturated ammonium chloride solution. The resulting solution was diluted with water and extracted with ethyl acetate. The organic layers were washed with brine, dried over anhydrous sodium sulfate and concentrated. The crude was used in the next step without purification. LCMS (ESI, m/z): 1044.4 [M+H]⁺.

Step 4: 4-((R)-1-((R)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyridazin-3-amine

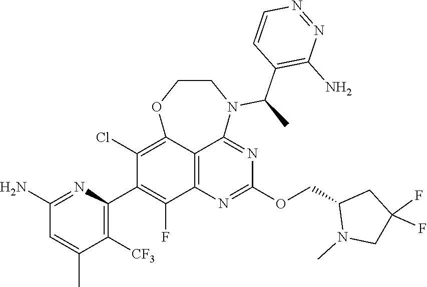

A mixture of 4-((R)-1-((R)-9-(6-(bis(4-methoxybenzyl)amino)-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)-N-(4-methoxybenzyl)pyridazin-3-amine (400.0 mg, 0.38 mmol) in trifluoroacetic acid (1 mL) and trifuoromethanesulfonic acid (0.1 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous Sodium sulfate and concentrated. The crude product was purified by Prep-HPLC with the following conditions: Xselect CSH C18 OBD Column 30*150 mm 5 μm, n; Mobile Phase A: water (0.1% FA), Mobile Phase B: acetonitrile; Flow rate: 60 mL/min; Gradient: 8% B to 34% B in 9 min; 254/220 nm; RT1: 9 min to afford 4-((R)-1-((R)-9-(6-amino-4-methyl-3-(trifluoromethyl)pyridin-2-yl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-4-yl)ethyl)pyridazin-3-amine (35.7 mg, 0.05 mmol, 13.6% yield). LCMS (ESI, m/z): 684.2 [M+H]⁺.

Example 85: ¹H NMR (300 MHz, Methanol-d₄, ppm) δ 8.55 (d, J=4.8 Hz, 1H), 7.64 (d, J=4.6 Hz, 1H), 6.62 (s, 1H), 6.44 (q, J=6.8 Hz, 1H), 4.63-4.40 (m, 4H), 3.87-3.80 (m, 1H), 3.65-3.58 (in, 1H), 3.48-3.34 (m, 1H), 3.23-3.03 (in, 1H), 2.96-2.70 (in, 1H), 2.68-2.58 (m, 4H), 2.46 (s, 3H), 2.45-2.24 (in, 1H), 1.72 (d, J=6.9 Hz, 3H).

Example 86: 6-((R)-4-((R)-(2-aminopyridin-3-yl)(cyclopropyl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

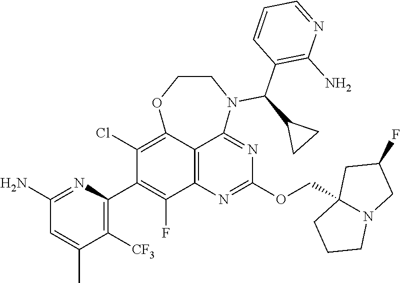

### Synthetic Route

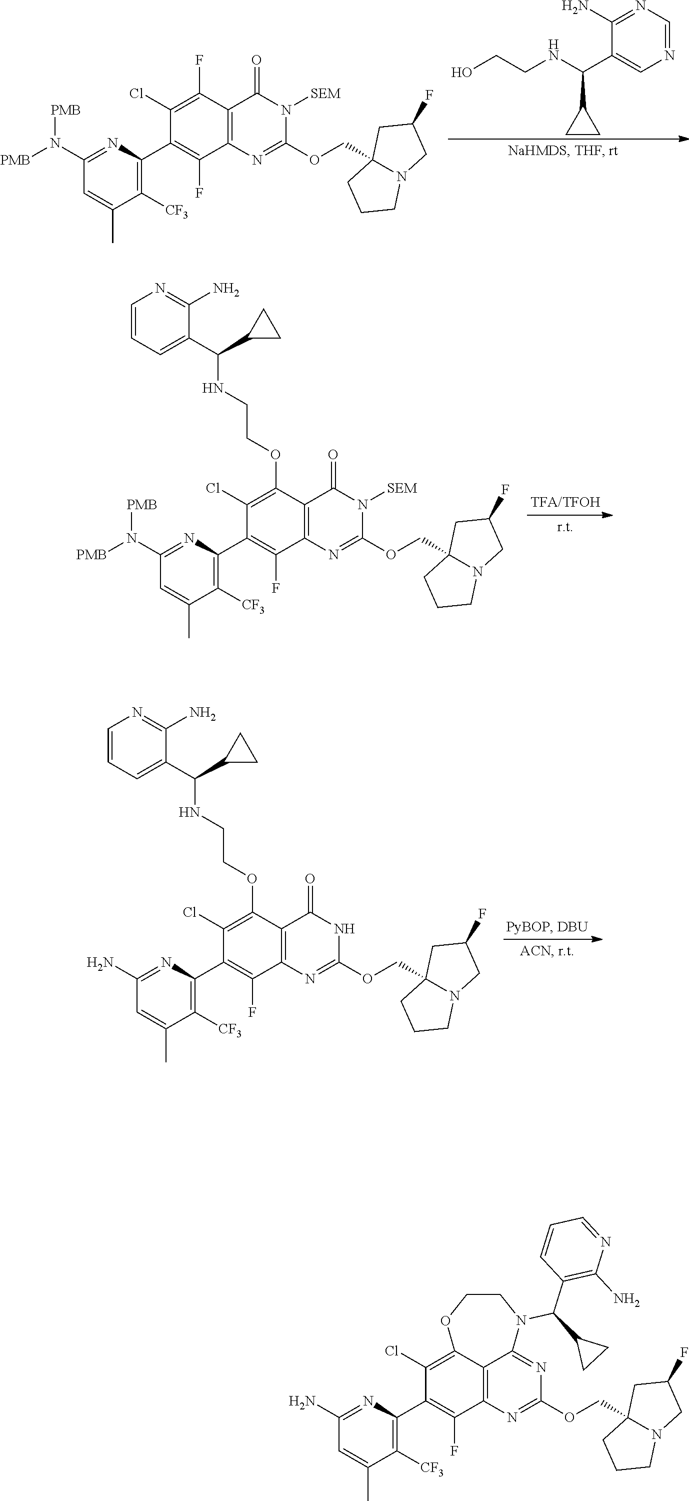

Step 1: (R)-5-(2-(((R)-(2-aminopyridin-3-yl)(cyclopropyl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one

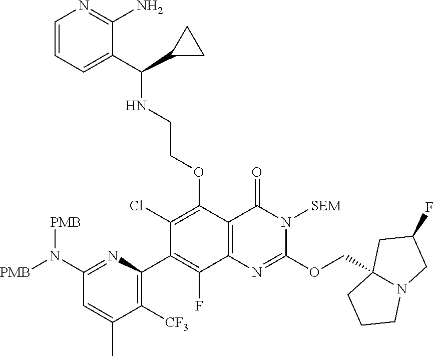

Under nitrogen, (R)-2-(((4-aminopyrimidin-5-yl)(cyclopropyl)methyl)amino)ethan-1-ol (294 mg, 1.42 mmol)) in tetrahydrofuran (10 mL) was added sodium bis(trimethylsilyl)amide (1.42 mL, 1.42 mmol, 1M in tetrahydrofuran) at 0° C. The reaction was stirred at 0° C. for 5 min. The mixture was transferred into a mixture of (R)-7-(6-(bis(4-methoxybenzyl)amino)-4-methyl-3-(trifluoromethyl)pyridin-2-yl)-6-chloro-5,8-difluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (1.00 g, 1.09 mmol) in tetrahydrofuran (10 mL) and stirred at room temperature for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (20:1) to afford (R)-5-(2-(((R)-(2-aminopyridin-3-yl)(cyclopropyl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (250 mg, 0.23 mmol, 21.1% yield) as a white solid. LCMS (ESI, m/z): 1105.4 [M+H]⁺.

Step 2: (R)-7-(6-amino-4-methyl-3-(trifluoromethyl)pyridin-2-yl)-5-(2-(((R)-(2-aminopyridin-3-yl)(cyclopropyl)methyl)amino)ethoxy)-6-chloro-8-fluoro-2-(((2R,7aS)-2-fluorotetrahydro-1H-pyrrolizin-7a(5H)-yl)methoxy)quinazolin-4(3H)-one

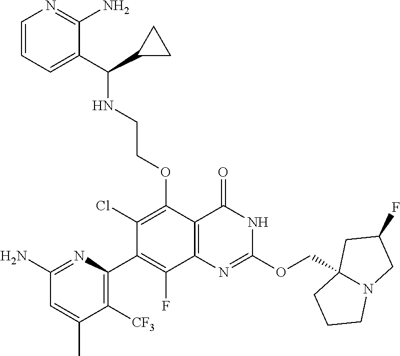

A mixture of (R)-5-(2-(((R)-(2-aminopyridin-3-yl)(cyclopropyl)methyl)amino)ethoxy)-7-(6-(bis(4-methoxybenzyl)amino)-4-methyl-3-(trifluoromethyl)pyridin-2-yl)-6-chloro-8-fluoro-2-(((2R,7aS)-2-fluorotetrahydro-1H-pyrrolizin-7a(5H)-yl)methoxy)-3-((2-(trimethylsilyl)ethoxy)methyl)quinazolin-4(3H)-one (250.0 mg, 0.23 mmol) in trifluoroacetic acid (5 mL) and trifuoromethanesulfonic acid (0.5 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The resulting residue was purified by reverse phase chromatography (acetonitrile/0.1% NH₄HCO₃ in water) to afford (R)-7-(6-amino-4-methyl-3-(trifluoromethyl)pyridin-2-yl)-5-(2-(((R)-(2-aminopyridin-3-yl)(cyclopropyl)methyl)amino)ethoxy)-6-chloro-8-fluoro-2-(((2R,7aS)-2-fluorotetrahydro-1H-pyrrolizin-7a(5H)-yl)methoxy)quinazolin-4(3H)-one (160 mg, 0.22 mmol, 96.3% yield) as a white solid. LCMS (ESI, m/z): 735.2 [M+H]⁺.

Step 3: 6-((R)-4-((R)-(2-aminopyridin-3-yl)(cyclopropyl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

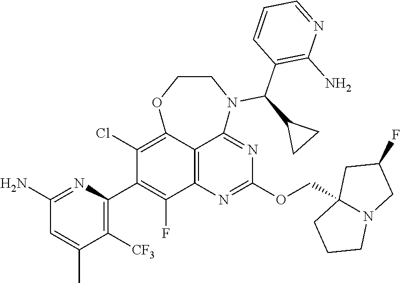

A solution of (R)-7-(6-amino-4-methyl-3-(trifluoromethyl)pyridin-2-yl)-5-(2-(((R)-(2-aminopyridin-3-yl)(cyclopropyl)methyl)amino)ethoxy)-6-chloro-8-fluoro-2-(((2R,7aS)-2-fluorotetrahydro-1H-pyrrolizin-7a(5H)-yl)methoxy)quinazolin-4(3H)-one (150.0 mg, 0.20 mmol), bis(2-oxo-3-oxazolidinyl)phosphinicchloride (150.0 mg, 0.59 mmol) and N,N-diisopropylethylamine (135.0 mg, 1.04 mmol) in chloroform (5 mL) was stirred at 70° C. for 2 hours. The solvent was removed under vacuum. The resulting residue was purified by reverse phase chromatography (acetonitrile/0.1% NH₄HCO₃ in water) to afford crude product. The crude product was purified by Prep-HPLC with the following conditions: Column: XBridge Prep C18 OBD Column, 30*100 mm, 5 μm; Mobile Phase A: Water (10 mmol/L NH₄HCO₃), Mobile Phase B: ACN: Flow rate: 60 mL/min; Gradient: 34% B to 62% B in 8 min, 62% B; Wave Length: 254/220 nm; RT1(min): 6.32 to afford 6-((R)-4-((R)-(2-aminopyridin-3-yl)(cyclopropyl)methyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (47.5 mg, 0.07 mmol, 32.5% yield). LCMS (ESI, m/z): 717.2 [M+H]⁺.

Example 86: ¹H NMR (400 MHz, DMSO-d₆, ppm) δ 7.97 (dd, J=4.8, 1.8 Hz, 1H), 7.88 (dd, J=7.5, 1.8 Hz, 1H), 6.81 (s, 2H), 6.68 (dd, J=7.5, 4.9 Hz, 1H), 6.47 (s, 1H), 5.79 (s, 2H), 5.44 (d, J=10.1 Hz, 1H), 5.27 (d, J=54.4 Hz, 1H), 4.66-4.47 (m, 1H), 4.38-4.24 (m, 1H), 4.16-3.94 (m, 2H), 3.94-3.79 (m, 1H), 3.64-3.48 (m, 1H), 3.15-2.91 (m, 3H), 2.91-2.72 (m, 1H), 2.36 (s, 3H), 2.20-1.92 (m, 3H), 1.90-1.65 (m, 4H), 0.80-0.27 (m, 4H).

Example 87a: 6-((R)-4-((R)-1-(2-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

Example 87b: 6-((R)-4-((R)-1-(2-aminopyridin-3-yl)ethyl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

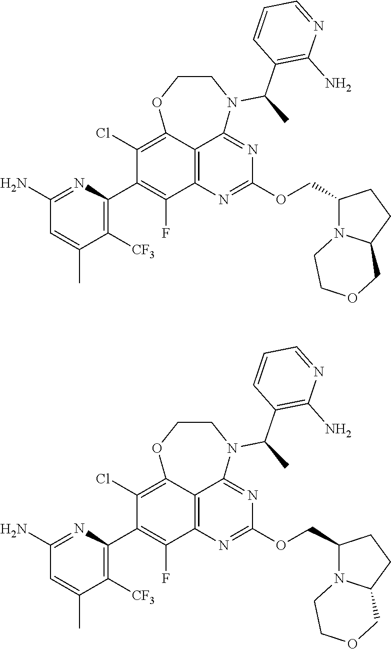

### Synthetic Route

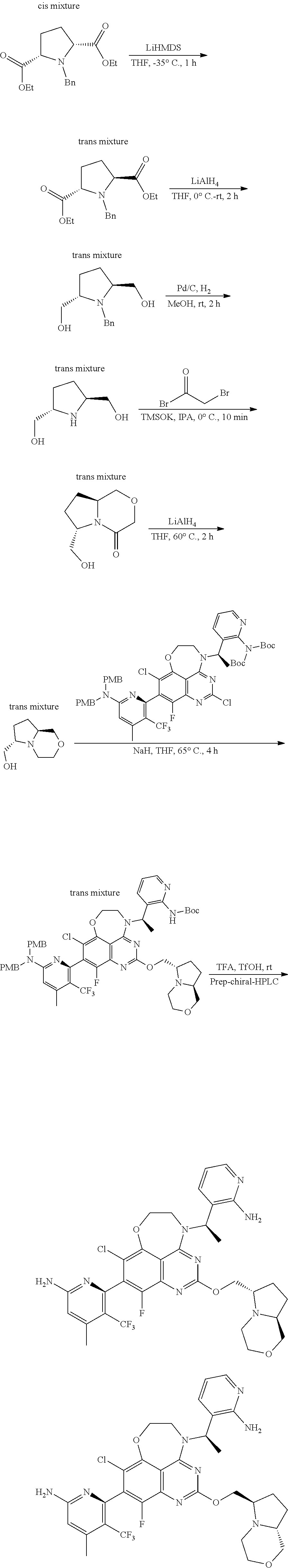

Step 1: diethyl (2S,5S)-1-benzylpyrrolidine-2,5-dicarboxylate (Trans Mixture)

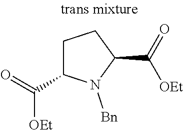 [SMILES: CCOC(=O)[C@@H]1CC[C@@H](C(=O)OCC)N1Cc1ccccc1]

To a mixture of diethyl (2S,5R)-1-benzylpyrrolidine-2,5-dicarboxylate hydrochloride (cis mixture) (50.0 g, 146.27 mmol) in tetrahydrofuran (500 mL) was added lithium bis(trimethylsilyl)amide (290 mL, 290 mmol, 1M in tetrahydrofuran), the mixture was stirred for 1 hour at −40° C., then lithium bis(trimethylsilyl)amide (146 mL, 146 mmol, 1M in tetrahydrofuran) was added and stirred for another 1 hour at −40° C. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (97:3) to afford diethyl (2S,5S)-1-benzylpyrrolidine-2,5-dicarboxylate (trans mixture) (4.0 g, 13.07 mmol, 9% yield) as a colorless oil. LC-MS: (ESI, m/z): [M+H]⁺⁼306.0.

Step 2: ((2S,5S)-1-benzylpyrrolidine-2,5-diyl)dimethanol (Trans Mixture)

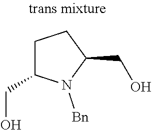 [SMILES: OC[C@@H]1CC[C@@H](CO)N1Cc1ccccc1]

To a mixture of diethyl (2S,5S)-1-benzylpyrrolidine-2,5-dicarboxylate (trans mixture) (17.7 g, 57.96 mmol) in tetrahydrofuran (200 mL) was added lithium aluminumhydride (115 mL, 230 mmol, 2M in tetrahydrofuran), the mixture was stirred for 1 hour at 0° C. The reaction was quenched with sodium sulfate decahydrate. The solids were filtered out. After filtration, the filtrate was concentrated under reduced pressure. The residue was purified by flash chromatography on silica gel eluting with dichloromethane/methanol (90:10) to afford ((2S,5S)-1-benzylpyrrolidine-2,5-diyl)dimethanol (trans mixture) (11.3 g, 51.06 mmol, 88.1% yield) as a yellow oil. LC-MS: (ESI, m/z): [M+H]⁺=222.3.

Step 3: ((2S,5S)-pyrrolidine-2,5-diyl)dimethanol (Trans Mixture)

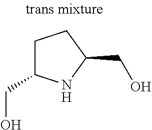

A mixture of ((2S,5S)-1-benzylpyrrolidine-2,5-diyl)dimethanol (trans mixture) (4.0 g, 18.08 mmol) and Palladium carbon (2.0 g, 1.89 mmol) in methyl alcohol (60 mL) was stirred at room temperature for 1 hour under hydrogen. The solids were filtered out. After filtration, the filtrate was concentrated under reduced pressure. The crude product was used in the next step without purification. LC-MS: (ESI, m/z): [M+H]⁺=132.1.

Step 4: (6S,8aS)-6-(hydroxymethyl)tetrahydro-1H-pyrrolo[2,1-c][1,4]oxazin-4(3H)-one (Trans Mixture)

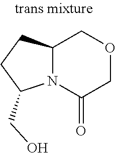 [SMILES: O=C1COC[C@@H]2CC[C@@H](CO)N12]

To a mixture of ((2S,5S)-pyrrolidine-2,5-diyl)dimethanol (trans mixture) (2.50 g, 19.06 mmol) in 2-propanol (3 mL) was added potassium trimethylsilanolate (4.90 g, 38.19 mmol) and bromoacetyl bromide (3.81 g, 19.07 mmol), the mixture was stirred for 10 min at 0° C. After completion, the solvent was removed under vacuum. The residue was purified by flash chromatography on silica gel eluting with dichloromethane/methanol (94:6) to afford (6S,8aS)-6-(hydroxymethyl)tetrahydro-1H-pyrrolo[2,1-c][1,4]oxazin-4(3H)-one (trans mixture) (1.50 g, 8.76 mmol, 46% yield) as a yellow oil. LCMS (ESI, m/z): 172.0 [M+H]⁺.

Step 5: ((6S,8aS)-hexahydro-1H-pyrrolo[2,1-c][1,4]oxazin-6-yl)methanol (Trans Mixture)

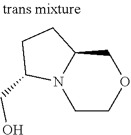 [SMILES: OC[C@@H]1CC[C@H]2COCCN12]

A mixture of (6S,8aS)-6-(hydroxymethyl)tetrahydro-1H-pyrrolo[2,1-c][1,4]oxazin-4(3H)-one (trans mixture) (1.30 g, 7.59 mmol) and lithium aluminumhydride (3.00 g, 79.04 mmol) in tetrahydrofuran (40 mL) was stirred at 60° C. for 2 hours. The reaction was quenched with 3 ml water, 3 ml 10% sodium hydroxide solution and 9 ml water. The solids were filtered out. After filtration, the filtrate was concentrated under reduced pressure. The residue was purified by flash chromatography on silica gel eluting with dichloromethane/methanol (80:20) to afford ((6S,8aS)-hexahydro-1H-pyrrolo[2,1-c][1,4]oxazin-6-yl)methanol (trans mixture) (692 mg, 4.40 mmol, 58% yield) as a brown oil.

Step 6: tert-butyl (3-((R)-1-((R)-9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyridin-2-yl)carbamate (Trans Mixture)

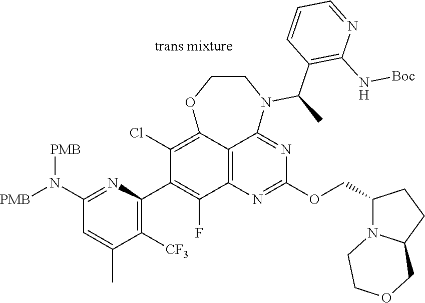

To a mixture of ((6S,8aS)-hexahydro-1H-pyrrolo[2,1-c][1,4]oxazin-6-yl)methanol (trans mixture) (650.0 mg, 4.13 mmol) in tetrahydrofuran (7 mL) was added sodium bis(trimethylsilyl)amide (5 mL, 5.0 mmol, 1M in tetrahydrofuran), the mixture was stirred for 10 min at room temperature. Then tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]carbamate (desired atropisomer) (750.0 mg, 0.83 mmol) in tetrahydrofuran (7 mL) was added, stirred for 1 hour at room temperature. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (90:10) to afford tert-butyl (3-((R)-1-((R)-9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyridin-2-yl)carbamate (trans mixture) (620 mg, 0.60 mmol, 73% yield) as a yellow solid.

Step 7: 6-((R)-4-((R)-1-(2-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

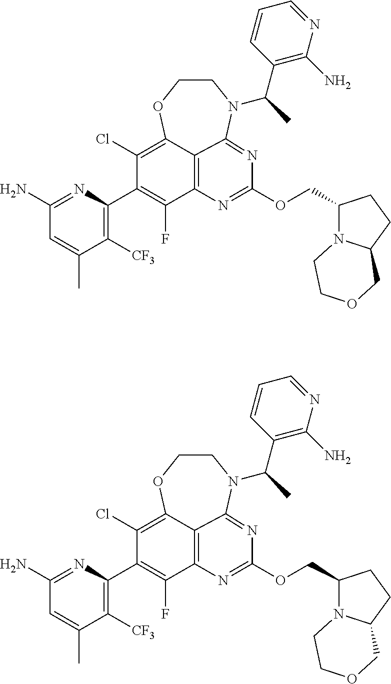

A solution of tert-butyl (3-((R)-1-((R)-9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyridin-2-yl)carbamate (trans mixture) (800.0 mg, 0.78 mmol) in trifluoroacetic acid (10 mL) and trifuoromethanesulfonic acid (1 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 27% B to 54% B in 10 min, 54% B; Wave Length: 254/220 nm; RT1(min): 9.18. The product was purified by Chiral-Prep-HPLC with the following conditions: Column: CHIRALPAK ID, 2*25 cm, 5 μm, Mobile Phase A: Hex:DCM=3:1(0.5% 2M NH₃-MeOH)-HPLC, Mobile Phase B: IPA-HPLC; Flow rate: 20 mL/min; Gradient: 85% B to 85% B in 22 min; Wave Length: 220/254 nm; RT1(min): 14.911; RT2(min): 18.469; Sample Solvent: IPA-HPLC; Injection Volume: 1.2 mL; Number of Runs: 2 to afford 6-((R)-4-((R)-1-(2-aminopyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (87.7 mg, 0.13 mmol, 16.4% yield) and 6-((R)-4-((R)-1-(2-aminopyridin-3-yl)ethyl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (101.5 mg, 0.15 mmol, 19% yield). The stereo chemistry of title compounds was arbitrarily assigned.

Example 87a: LCMS (ESI, m/z): 689.2 [M+H]⁺. ¹H NMR (300 MHz, DMSO-d₆, ppm) δ 7.97 (dd, J=4.9, 1.7 Hz, 1H), 7.63 (dd, J=7.6, 1.8 Hz, 1H), 6.82 (s, 2H), 6.67 (dd, J=7.5, 4.9 Hz, 1H), 6.48 (s, 1H), 6.24 (q, J=6.8 Hz, 1H), 5.70 (s, 2H), 4.50-4.37 (m, 2H), 4.27 (dd, J=11.9, 6.7 Hz, 1H), 4.16 (dd, J=10.9, 5.8 Hz, 1H), 3.72-3.55 (m, 3H), 3.54-3.40 (m, 3H), 3.14 (t, J=10.3 Hz, 1H), 3.04-2.78 (m, 3H), 2.36 (s, 3H), 2.18-1.99 (m, 1H), 1.85-1.70 (m, 1H), 1.69-1.59 (m, 1H), 1.57 (d, J=6.7 Hz, 3H), 1.39-1.22 (m, 1H). Chiral HPLC: Column: CHIRALPAK ID-3, 4.6*50 mm, 3 um; (Hex:DCM=3:1)(0.1% DEA):IPA=85:15; Flow rate: 1 mL/min; Retention time: 2.804 (faster peak).

Example 87b: LCMS (ESI, m/z): 689.3 [M+H]⁺. ¹H NMR (300 MHz, DMSO-d₆, ppm) δ 7.97 (dd, J=4.9, 1.7 Hz, 1H), 7.63 (d, J=7.0 Hz, 1H), 6.82 (s, 2H), 6.67 (dd, J=7.5, 4.9 Hz, 1H), 6.48 (s, 1H), 6.25 (q, J=6.9 Hz, 1H), 5.73 (s, 2H), 4.51-4.35 (m, 2H), 4.27 (dd, J=11.8, 6.7 Hz, 1H), 4.14 (dd, J=10.9, 5.7 Hz, 1H), 3.74-3.56 (m, 3H), 3.56-3.39 (m, 3H), 3.14 (t, J=10.4 Hz, 1H), 3.05-2.77 (m, 3H), 2.36 (s, 3H), 2.17-1.98 (m, 1H), 1.85-1.69 (m, 1H), 1.68-1.60 (m, 1H), 1.56 (d, J=6.9 Hz, 3H), 1.41-1.18 (m, 1H). Chiral HPLC: Column: CHIRALPAK ID-3, 4.6*50 mm, 3 um. (Hex:DCM=3:1)(0.1% DEA):IPA=85:15; Flow rate: 1 mL/min; Retention time: 3.674 (slower peak).

Example 88: (R)-6-(4-((R)-1-(2-aminopyridin-3-yl)ethyl)-8-bromo-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

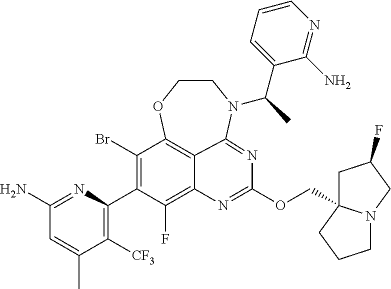

### Synthetic Route

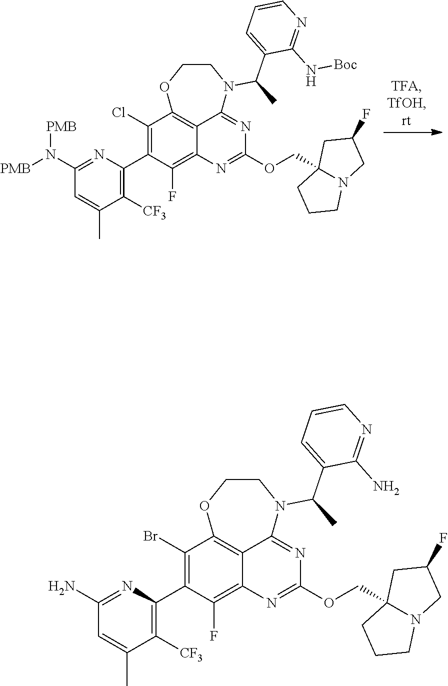

Step 1: (R)-6-(4-((R)-1-(2-aminopyridin-3-yl)ethyl)-8-bromo-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

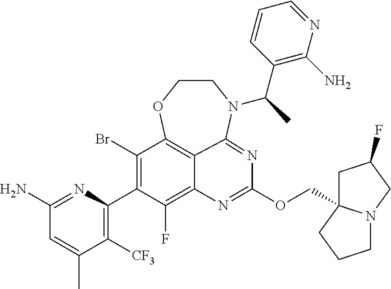

A solution of tert-butyl N-[3-[(1R)-1-[7-[6-[bis[(4-methoxyphenyl)methyl]amino]-4-methyl-3-(trifluoromethyl)-2-pyridyl]-8-chloro-6-fluoro-3-[[(2R,8S)-2-fluoro-1,2,3,5,6,7-hexahydropyrrolizin-8-yl]methoxy]-10-oxa-2,4,13-triazatricyclo[7.4.1.05,14]tetradeca-1,3,5,7,9(14)-pentaen-13-yl]ethyl]-2-pyridyl]carbamate (100.0 g, 96.95 mmol) in trifluoroacetic acid (700 mL) and trifuoromethanesulfonic acid (70 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (90:10) to afford (R)-6-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (24.6 g, 35.60 mmol, 36.7% yield).

(R)-6-(4-((R)-1-(2-aminopyridin-3-yl)ethyl)-8-bromo-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (36.3 mg, 0.05 mmol) was isolated. LC-MS: (ESI, m/z): 660.3[M+H]⁺

Example 88: ¹H NMR (300 MHz, Methanol-d₄, ppm) δ 7.85 (dd, J=5.1, 1.7 Hz, 1H), 7.69-7.61 (m, 1H), 6.67 (dd, J=7.5, 5.1 Hz, 1H), 6.48 (s, 1H), 6.38 (q, J=6.8 Hz, 1H), 5.20 (d, J=53.8 Hz, 1H), 4.40-4.27 (m, 1H), 4.24-4.10 (m, 3H), 3.63-3.50 (m, 1H), 3.46-3.33 (m, 1H), 3.17-3.03 (m, 3H), 2.97-2.82 (m, 1H), 2.33 (d, J=2.1, 3H), 2.30-1.99 (m, 3H), 1.96-1.72 (m, 3H), 1.55 (d, J=6.9 Hz, 3H).

Example 89: 6-((R)-4-((R)-1-(2-aminopyridin-3-yl)ethyl)-8-chloro-2-(((2R,7aS)-2-ethoxytetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine

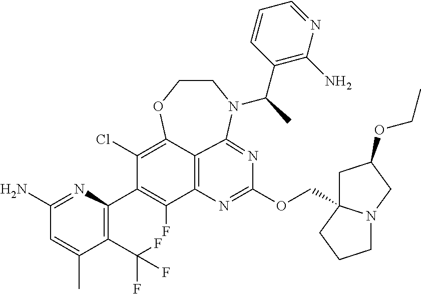

### Synthetic Route

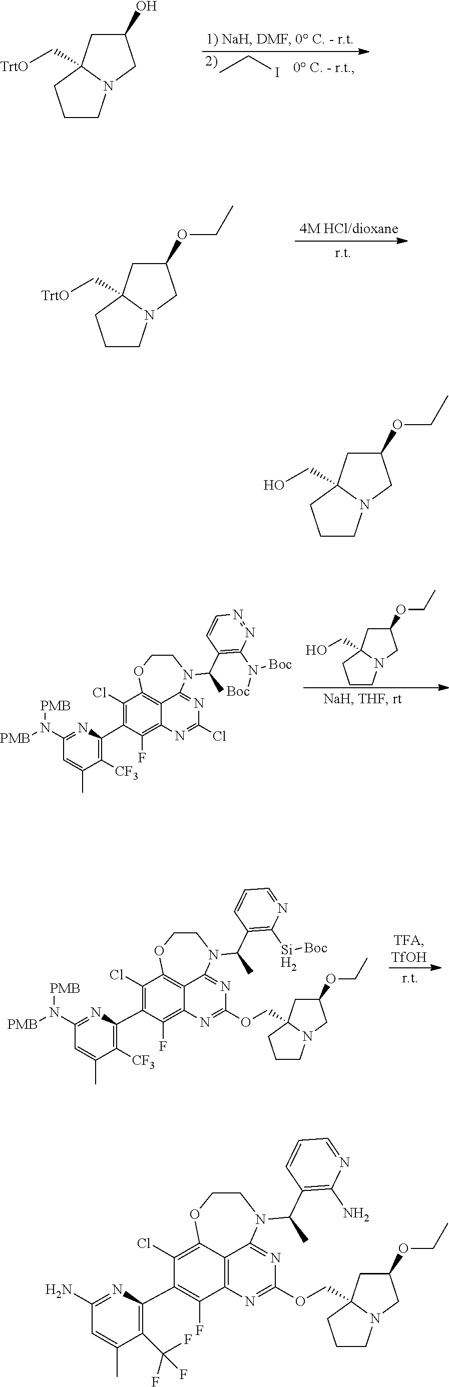

Step 1: (2R,7aS)-2-ethoxy-7a-((trityloxy)methyl)hexahydro-1H-pyrrolizine

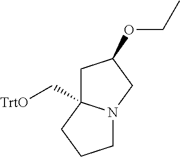

To a solution of (2R,7aS)-7a-((trityloxy)methyl)hexahydro-1H-pyrrolizin-2-ol (800.0 mg, 2 mmol) in N,N-dimethylformamide (8 mL) was added sodium hydride (400.0 mg, 10 mmol, 60% dispersion in mineral oil), the mixture was stirred at 0° C. for 10 minutes. Then iodoethane (937.0 mg, 6.0 mmol) was added and stirred at 25° C. for 2 hours. After completion, the resulting solution was quenched with saturated ammonium chloride solution and extracted with ethyl acetate. The organic layers were combined, washed with brine, dried over anhydrous sodium sulfate and concentrated under vacuum. The residue was purified by flash chromatography on silica gel eluting with petroleum ether/ethyl acetate (80:20) to afford (2R,7aS)-2-ethoxy-7a-((trityloxy)methyl)hexahydro-1H-pyrrolizine (800 mg, 1.76 mmol, 87.8% yield) as a colorless oil. LC-MS: (ESI, m/z): 428.2 [M+H]⁺

Step 2: ((2R,7aS)-2-ethoxytetrahydro-1H-pyrrolizin-7a(5H)-yl)methanol

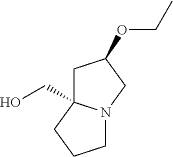

A solution of (2R,7aS)-2-ethoxy-7a-((trityloxy)methyl)hexahydro-1H-pyrrolizine (790.0 mg, 1.85 mmol) and 4M hydrochloric acid in 1,4-dioxane (4 mL) was stirred at 25° C. for 30 minutes. After completion, the solvent was removed under vacuum. The residue was purified by flash chromatography on silica gel eluting with dichloromethane/methanol (90:10) to afford ((2R,7aS)-2-ethoxytetrahydro-1H-pyrrolizin-7a(5H)-yl)methanol (140 mg, 0.76 mmol, 40.9% yield) as a white solid. LC-MS: (ESI, m/z): 186.1 [M+H]⁺

Step 3: tert-butyl (3-((R)-1-(9-((R)-6-(bis(4-methoxybenzyl)amino)-4-methyl-3-(trifluoromethyl)pyridin-2-yl)-8-chloro-2-(((2R,7aS)-2-ethoxytetrahydro-1H-pyrrolizin-7a(5H)-yl)methoxy)-10-fluoro-5,6-dihydro-4H-[1,4]oxazepino[5,6,7-de]quinazolin-4-yl)ethyl)pyridin-2-yl)silanecarboxylate

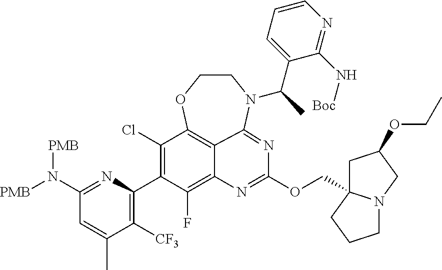

To a mixture of ((2R,7aS)-2-ethoxytetrahydro-1H-pyrrolizin-7a(5H)-yl)methanol (60.0 mg, 0.3200 mmol) in tetrahydrofuran (3 mL) was added Sodium hydride (12.0 mg, 0.50 mmol, 60% dispersion in mineral oil), the mixture was stirred for 10 minutes at 0° C. Then tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]-N-tert-butoxycarbonyl-carbamate (desired atropisomer) (100.0 mg, 0.10 mmol) in tetrahydrofuran (0.5 mL) was added, the mixture was stirred for 1 hour 65° C. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (92:8) to afford tert-butyl (3-((R)-1-(9-((R)-6-(bis(4-methoxybenzyl)amino)-4-methyl-3-(trifluoromethyl)pyridin-2-yl)-8-chloro-2-(((2R,7aS)-2-ethoxytetrahydro-1H-pyrrolizin-7a(5H)-yl)methoxy)-10-fluoro-5,6-dihydro-4H-[1,4]oxazepino[5,6,7-de]quinazolin-4-yl)ethyl)pyridin-2-yl)silanecarboxylate (77 mg, 0.07 mmol, 66.2% yield) as a white solid. LC-MS: (ESI, m/z): 1157.4 [M+H]⁺

Step 4: 6-((R)-4-((R)-1-(2-aminopyridin-3-yl)ethyl)-8-chloro-2-(((2R,7aS)-2-ethoxytetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine

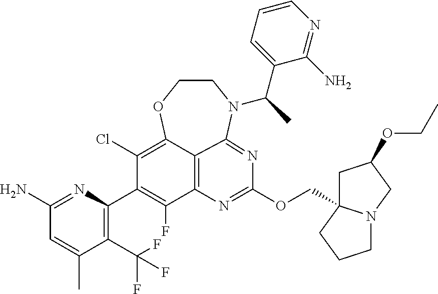

A solution of tert-butyl (3-((R)-1-(9-((R)-6-(bis(4-methoxybenzyl)amino)-4-methyl-3-(trifluoromethyl)pyridin-2-yl)-8-chloro-2-(((2R,7aS)-2-ethoxytetrahydro-1H-pyrrolizin-7a(5H)-yl)methoxy)-10-fluoro-5,6-dihydro-4H-[1,4]oxazepino[5,6,7-de]quinazolin-4-yl)ethyl)pyridin-2-yl)silanecarboxylate (70.0 mg, 0.06 mmol) in trifluoroacetic acid (5 mL) and trifuoromethanesulfonic acid (0.5 mL) was stirred at room temperature for 10 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 33% B to 58% B in 9 min, 58% B: Wave Length: 254/220 nm; RT1(min): 7.52 to afford 6-((R)-4-((R)-1-(2-aminopyridin-3-yl)ethyl)-8-chloro-2-(((2R,7aS)-2-ethoxytetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine (14.2 mg, 0.02 mmol, 32.4% yield). LC-MS: (ESI, m/z): 717.2[M+H]⁺

Example 89: ¹H NMR (300 MHz, DMSO-d₆, ppm) δ 7.98 (dd, J=4.9, 1.7 Hz, 1H), 7.68-7.59 (m, 1H), 6.81 (s, 2H), 6.67 (dd, J=7.5, 4.9 Hz, 1H), 6.48 (s, 1H), 6.33-6.18 (m, 1H), 5.72 (s, 2H), 4.43 (dd, J=11.9, 6.1 Hz, 1H), 4.25 (dd, J=11.9, 6.8 Hz, 1H), 4.15-3.97 (m, 3H), 3.64 (dd, J=15.9, 6.9 Hz, 1H), 3.49-3.34 (m, 3H), 3.11 (dd, J=10.5, 4.9 Hz, 1H), 3.00-2.88 (m, 1H), 2.81-2.69 (m, 1H), 2.63 (dd, J=10.5, 5.3 Hz, 1H), 2.36 (d, J=2.2 Hz, 3H), 2.18 (dd, J=13.0, 5.9 Hz, 1H), 1.96-1.88 (m, 2H), 1.87-1.62 (m, 3H), 1.57 (d, J=6.8 Hz, 3H), 1.08 (t, J=7.0 Hz, 3H).

Example 90: 6-((R)-4-((R)-1-(2-aminopyridin-3-yl)ethyl)-8-chloro-10-fluoro-2-(8-oxa-2,5-diazaspiro[3.5]nonan-2-yl)-5,6-dihydro-4H-[1,4]oxazepino[5,6,7-de]quinazolin-9-yl)-4-methyl-5-(trifluoromethyl)pyridin-2-amine

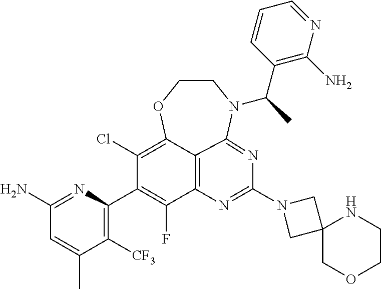

### Synthetic Route

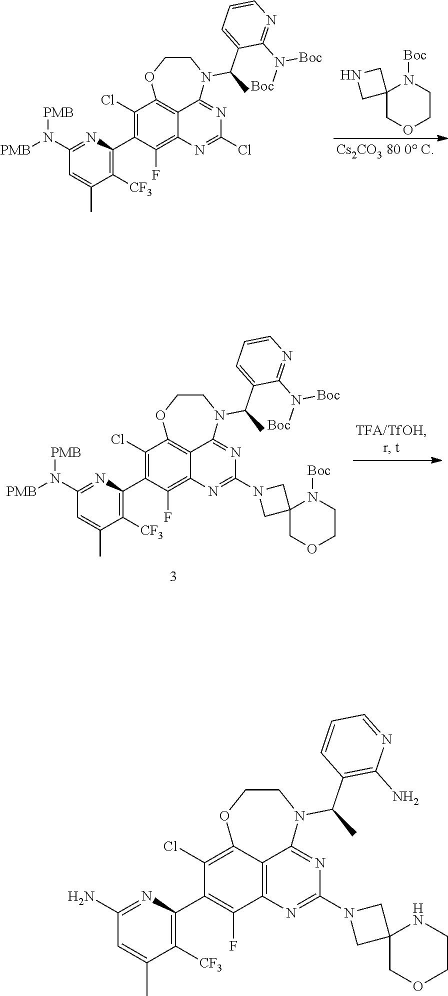

Step 1: tert-butyl 2-[13-[(1R)-1-[2-[bis(tert-butoxycarbonyl)amino]-3-pyridyl]ethyl]-7-[6-[bis[(4-methoxyphenyl)methyl]amino]-4-methyl-3-(trifluoromethyl)-2-pyridyl]-8-chloro-6-fluoro-10-oxa-2,4,13-triazatricyclo[7.4.1.05,14]tetredeca-1,3,5,7,9(14)-pentaen-3-yl]-8-oxa-2,5-diazaspiro[3.5]nonane-5-carboxylate

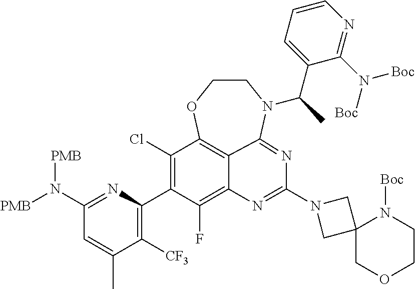

Under nitrogen, a mixture of tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]-N-tert-butoxycarbonyl-carbamate (150.0 mg, 0.15 mmol), tert-butyl 8-oxa-2,5-diazaspiro[3.5]nonane-5-carboxylate (51.0 mg, 0.22 mmol) and cesium carbonate (145.0 mg, 0.45 mmol) in dimethyl sulfoxide (5 mL) was stirred at 80° C. for 1 hour. After completion, the resulting solution was diluted with water and extracted with ethyl acetate. The organic layers were combined, washed with brine, dried over anhydrous sodium sulfate and concentrated under vacuum. The residue was purified by flash chromatography on silica gel eluting with petroleum ether/ethyl acetate (1:1) to afford tert-butyl 2-[13-[(1R)-1-[2-[bis(tert-butoxycarbonyl)amino]-3-pyridyl]ethyl]-7-[6-[bis[(4-methoxyphenyl)methyl]amino]-4-methyl-3-(trifluoromethyl)-2-pyridyl]-8-chloro-6-fluoro-10-oxa-2,4,13-triazatricyclo[7.4.1.05,14]tetradeca-1,3,5,7,9(14)-pentaen-3-yl]-8-oxa-2,5-diazaspiro[3.5]nonane-5-carboxylate (140 mg, 0.11 mmol, 75.1% yield) as a yellow solid. LC-MS: (ESI, m/z): 1201.0 [M+H]⁺

Step 2: 6-((R)-4-((R)-1-(2-aminopyridin-3-yl)ethyl)-8-chloro-10-fluoro-2-(8-oxa-2,5-diazaspiro[3.5]nonan-2-yl)-5,6-dihydro-4H-[1,4]oxazepino[5,6,7-de]quinazolin-9-yl)-4-methyl-5-(trifluoromethyl)pyridin-2-amine

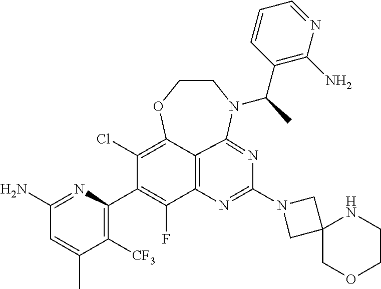

A solution of tert-butyl 2-[13-[(1R)-1-[2-[bis(tert-butoxycarbonyl)amino]-3-pyridyl]ethyl]-7-[6-[bis[(4-methoxyphenyl)methyl]amino]-4-methyl-3-(trifluoromethyl)-2-pyridyl]-8-chloro-6-fluoro-10-oxa-2,4,13-triazatricyclo[7.4.1.05,14]tetradeca-1,3,5,7,9(14)-pentaen-3-yl]-8-oxa-2,5-diazaspiro[3.5]nonane-5-carboxylate (140.0 mg, 0.12 mmol) in trifluoroacetic acid (5 mL) and trifuoromethanesulfonic acid (0.5 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 19*250 mm, 5 μm; Mobile Phase A: Water (10 mmol/L NH₄HCO₃), Mobile Phase B: ACN; Flow rate: 25 mL/min; Gradient: 70% B to 85% B in 10 min, 85% B; Wave Length: 254/220 nm: RT1(min): 5.23 to afford 6-((R)-4-((R)-1-(2-aminopyridin-3-yl)ethyl)-8-chloro-10-fluoro-2-(8-oxa-2,5-diazaspiro[3.5]nonan-2-yl)-5,6-dihydro-4H-[1,4]oxazepino[5,6,7-de]quinazolin-9-yl)-4-methyl-5-(trifluoromethyl)pyridin-2-amine (14.6 mg, 0.02 mmol, 18.7% yield). LC-MS: (ESI, m/z): 660.3[M+H]⁺

Example 90: ¹H NMR (300 MHz, DMSO-d₆, ppm) δ 7.94 (dd, J=4.9, 1.7 Hz, 1H), 7.60 (dd, J=7.5, 1.8 Hz, 1H), 6.75 (s, 2H), 6.64 (dd, J=7.4, 4.9 Hz, 1H), 6.47-6.40 (m, 1H), 6.26 (q, J=6.7 Hz, 1H), 5.81 (s, 2H), 4.47-4.26 (m, 1H), 4.21-4.03 (m, 1H), 3.98-3.74 (m, 4H), 3.62-3.37 (m, 5H), 3.29-3.15 (m, 2H), 2.73-2.72 (m, 2H), 2.33 (s, 3H), 1.51 (d, J=6.8 Hz, 3H).

Example 91a: 6-((R)-4-((R)-1-(2-aminopyridin-3-yl)ethyl)-8-chloro-2-(((6S,8aS)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-9-yl)-4-methyl-5-(trifluoromethyl)pyridin-2-amine

Example 91b: 6-((R)-4-((R)-1-(2-aminopyridin-3-yl)ethyl)-8-chloro-2-(((6R,8aR)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-9-yl)-4-methyl-5-(trifluoromethyl)pyridin-2-amine

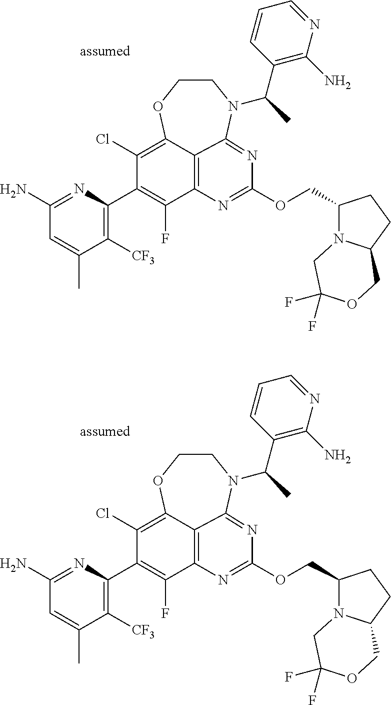

### Synthetic Route

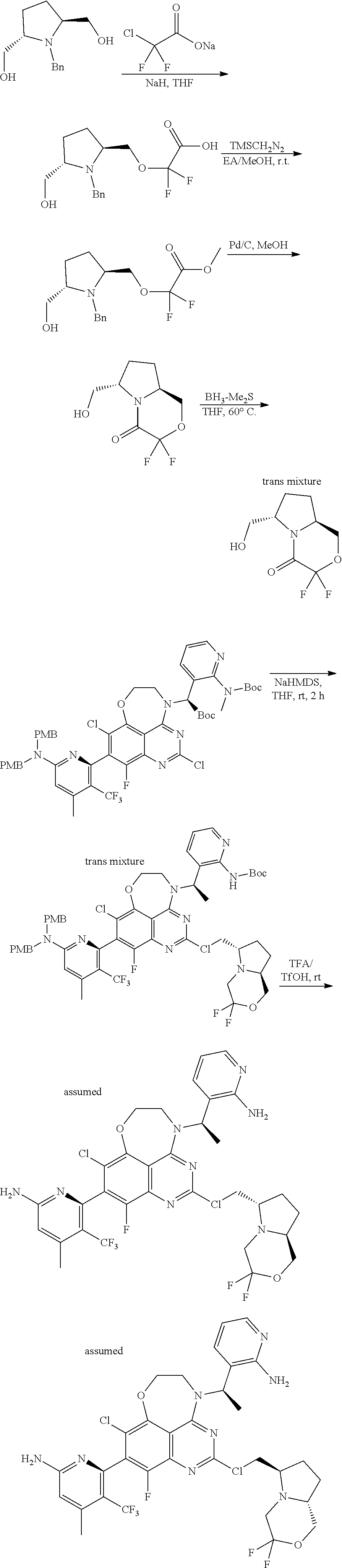

Step 1: 2-(((2S,5S)-1-benzyl-5-(hydroxymethyl)pyrrolidin-2-yl)methoxy)-2,2-difluoroacetic acid

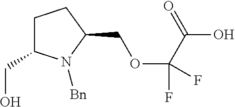 [SMILES: O=C(O)C(F)(F)OC[C@@H]1CC[C@@H](CO)N1Cc1ccccc1]

To a mixture of [(2S,5S)-1-benzyl-5-(hydroxymethyl)pyrrolidin-2-yl]methanol (3.00 g, 13.56 mmol) in tetrahydrofuran (50 mL) was added sodium hydride (2.16 g, 54.01 mmol, 60% dispersion in mineral oil), the mixture was stirred for 10 min at 0° C. Then (2-chloro-2,2-difluoro-acetyl)oxysodium (2.40 g, 15.74 mmol) was added and stirred at 60° C. for 1 hour. The reaction was quenched with 1M hydrochloric acid. The residue was purified by flash chromatography on silica gel eluting with dichloromethane/methanol (85:15) to afford 2-(((2S,5S)-1-benzyl-5-(hydroxymethyl)pyrrolidin-2-yl)methoxy)-2,2-difluoroacetic acid (800 mg, 2.54 mmol, 18.7% yield) as a brown oil. LCMS (ESI, m/z): 316.1 [M+H]⁺.

Step 2: methyl 2-(((2S,5S)-1-benzyl-5-(hydroxymethyl)pyrrolidin-2-yl)methoxy)-2,2-difluoroacetate

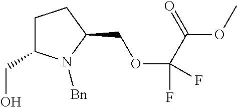 [SMILES: COC(=O)C(F)(F)OC[C@@H]1CC[C@@H](CO)N1Cc1ccccc1]

To a mixture of 2-(((2S,5S)-1-benzyl-5-(hydroxymethyl)pyrrolidin-2-yl)methoxy)-2,2-difluoroacetic acid (800.0 mg, 2.54 mmol) in methyl alcohol (5 mL) and dichloromethane (5 mL) was added (trimethylsilyl)diazomethane (4.0 mL, 25.14 mmol), the mixture was stirred for 1 hour at room temperature. After completion, the solvent was removed under vacuum. The crude product was used in the next step without purification.

LCMS (ESI, m/z): 330.1 [M+H]⁺.

Step 3: (6S,8aS)-3,3-difluoro-6-(hydroxymethyl)tetrahydro-1H-pyrrolo[2,1-c][1,4]oxazin-4(3H)-one

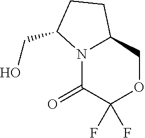 [SMILES: O=C1N2[C@H](CO)CC[C@H]2COC1(F)F]

To a solution of methyl 2-(((2S,5S)-1-benzyl-5-(hydroxymethyl)pyrrolidin-2-yl)methoxy)-2,2-difluoroacetate (800.0 mg, 2.43 mmol) in methanol (10 mL) was added palladium carbon (500.0 mg, 0.47 mmol), the mixture was stirred at room temperature for 2 hours under hydrogen. The solids were filtered out. After filtration, the filtrate was concentrated under reduced pressure. The residue was purified by flash chromatography on silica gel eluting with dichloromethane/methanol (95:5) to afford (6S,8aS)-3,3-difluoro-6-(hydroxymethyl)tetrahydro-1H-pyrrolo[2,1-c][1,4]oxazin-4(3H)-one (230 mg, 1.11 mmol, 45.7% yield) as a colorless oil. LCMS (ESI, m/z): 208.1 [M+H]⁺.

Step 4: ((6S,8aS)-3,3-difluorohexahydro-1H-pyrrolo[2,1-c][1,4]oxazin-6-yl)methanol

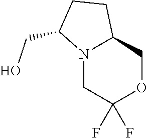 [SMILES: OC[C@@H]1CC[C@H]2COC(F)(F)CN12]

To a mixture of (6S,8aS)-3,3-difluoro-6-(hydroxymethyl)tetrahydro-1H-pyrrolo[2,1-c][1,4]oxazin-4(3H)-one (190.0 mg, 0.92 mmol) in tetrahydrofuran (5 mL) was added borane dimethylsulfide complex (210.0 mg, 2.76 mmol), the mixture was stirred for 1 h at 60° C. The reaction was quenched with methanol. The solvent was removed under vacuum. The crude was used directly without further purification. LCMS (ESI, m/z): 194.1 [M+H]⁺.

Step 5: tert-butyl (3-((R)-1-((R)-9-(6-(bis(4-methoxybenzyl)amino)-4-methyl-3-(trifluoromethyl)pyridin-2-yl)-8-chloro-2-(((6S,8aS)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-4-yl)ethyl)pyridin-2-yl)carbamate

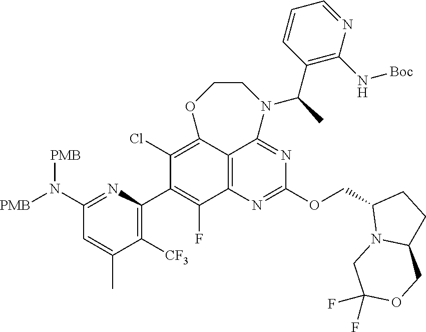

Under nitrogen, to a solution of ((6S,8aS)-3,3-difluorohexahydro-1H-pyrrolo[2,1-c][1,4]oxazin-6-yl)methanol (200.0 mg, 1.04 mmol) in tetrahydrofuran (8 mL) was added sodium bis(trimethylsilyl)amide (1.2 mL, 1.2 mmol, 1M in tetrahydrofuran), the mixture was stirred for 10 min at room temperature. Then tert-butyl N-[3-[(1R)-1-[7-[6-[bis[(4-methoxyphenyl)methyl]amino]-4-methyl-3-(trifluoromethyl)-2-pyridyl]-8-chloro-6-fluoro-3-[[(6S,8S)-4-oxa-1-azabicyclo[4.2.0]octan-8-yl]methoxy]-10-oxa-2,4,13-triazatricyclo[7.4.1.05,14]tetradeca-1,3,5(14),6,8-pentaen-13-yl]ethyl]-2-pyridyl]carbamate (400.0 mg, 0.40 mmol) was added and stirred at room temperature for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with petroleum ether/ethyl acetate (30:70) to afford tert-butyl (3-((R)-1-((R)-9-(6-(bis(4-methoxybenzyl)amino)-4-methyl-3-(trifluoromethyl)pyridin-2-yl)-8-chloro-2-(((6S,8aS)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-4-yl)ethyl)pyridin-2-yl)carbamate (220 mg, 0.22 mmol, 54.6% yield) as a yellow solid. LCMS (ESI, m/z): 1065.4 [M+H]⁺.

Step 6: 6-((R)-4-((R)-1-(2-aminopyridin-3-yl)ethyl)-8-chloro-2-(((6S,8aS)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-2-(((6R,8aR)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-9-yl)-4-methyl-5-(trifluoromethyl)pyridin-2-amine

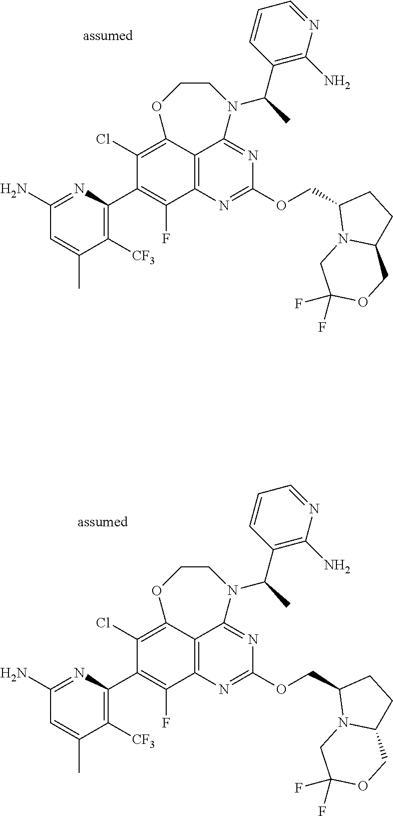

A mixture of tert-butyl (3-((R)-1-((R)-9-(6-(bis(4-methoxybenzyl)amino)-4-methyl-3-(trifluoromethyl)pyridin-2-yl)-8-chloro-2-(((6S,8aS)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-4-yl)ethyl)pyridin-2-yl)carbamate (200.0 mg, 0.1900 mmol) in in trifluoroacetic acid (5 mL) and trifuoromethanesulfonic acid (0.5 mL) was stirred at room temperature for 5 min. After completion, the resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water (10 mmol/L NH₄HCOs), Mobile Phase B: ACN; Flow rate: 60 mL/min; Gradient: 39% B to 64% B in 9 min, 64% B: Wave Length: 254/220 nm; RT(min): 8.6 to afford crude product. The product was purified by Chiral-Prep-HPLC with the following conditions: Column: CHIRALPAK IE, 2*25 cm, 5 um; Mobile Phase A: Hex:DCM=3:1(0.5% 2M NH3-MeOH)-HPLC, Mobile Phase B: EtOH-HPLC; Flow rate: 20 mL/min; Gradient: 30% B to 30% B in 17 min; Wave Length: 220/254 nm; RT1(min): 12.05; RT2(min): 14.89; Sample Solvent: EtOH-HPLC; Injection Volume: 0.3 mL; Number Of Runs: 8 to afford 6-((R)-4-((R)-1-(2-aminopyridin-3-yl)ethyl)-8-chloro-2-(((6S,8aS)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-9-yl)-4-methyl-5-(trifluoromethyl)pyridin-2-amine (22 mg, 0.03 mmol, 16.2% yield) and 6-((R)-4-((R)-1-(2-aminopyridin-3-yl)ethyl)-8-chloro-2-(((6R,8aR)-3,3-difluorohexahydro-1H-pyrrolo[2,1-c][1,4]oxazin-6-yl)methoxy)-10-fluoro-5,6-dihydro-4H-[1,4]oxazepino[5,6,7-de]quinazolin-9-yl)-4-methyl-5-(trifluoromethyl)pyridin-2-amine (26.7 mg, 0.04 mmol, 19.6% yield). The stereo chemistry of title compounds was arbitrarily assigned.

Example 91a: LCMS (ESI, m/z): 725.2 [M+H]⁺. ¹H NMR (300 MHz, Methanol-d₄, ppm) δ 7.96 (d, J=4.9 Hz, 1H), 7.75 (d, J=7.5 Hz, 1H), 6.85-6.70 (m, 1H), 6.59 (s, 1H), 6.50 (q, J=6.8 Hz, 1H), 4.58-4.36 (m, 3H), 4.31-4.22 (m, 1H), 4.02-3.79 (m, 2H), 3.79-3.34 (m, 4H), 3.28-3.10 (m, 2H), 2.44 (d, J=2.5 Hz, 3H), 2.31-2.14 (m, 1H), 2.08-1.92 (m, 1H), 1.85-1.70 (m, 1H), 1.66 (d, J=6.9 Hz, 3H), 1.61-1.44 (m, 1H). Chiral HPLC: Column: CHIRALPAK IE-3, 4.6*50 mm, 3 um; (Hex:DCM=3:1)(0.1% DEA):EtOH=70:30; Flow rate: 1 mL/min; Retention time: 3.022 (faster peak).

Example 91b: LCMS (ESI, m/z): 725.2 [M+H]⁺. ¹H NMR (300 MHz, Methanol-d₄, ppm) δ 7.95 (d, J=5.0 Hz, 1H), 7.74 (d, J=7.5 Hz, 1H), 6.85-6.70 (m, 1H), 6.59 (s, 1H), 6.51 (q, J=6.8 Hz, 1H), 4.55-4.36 (m, 3H), 4.36-4.21 (m, 1H), 4.04-3.79 (m, 2H), 3.79-3.34 (m, 5H), 3.29-3.12 (m, 1H), 2.44 (s, 3H), 2.30-2.13 (m, 1H), 2.07-1.91 (m, 1H), 1.84-1.69 (m, 1H), 1.66 (d, J=6.8 Hz, 3H), 1.61-1.45 (m, 1H). Chiral HPLC: Column: CHIRALPAK IE-3, 4.6*50 mm, 3 um; (Hex:DCM=3:1)(0.1% DEA):EtOH=70:30; Flow rate: 1 mL/min; Retention time: 3.961 (slower peak).

Example 92a: 6-((R)-2-(((1R,5R,7S)-4-oxa-1-azabicyclo[3.2.1]octan-7-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

Example 92b: 6-((R)-2-(((1R,5R,7R)-4-oxa-1-azabicyclo[3.2.1]octan-7-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

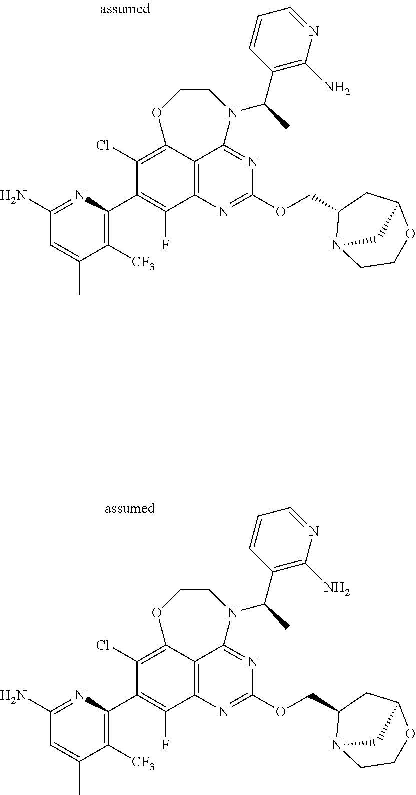

### Synthetic Route

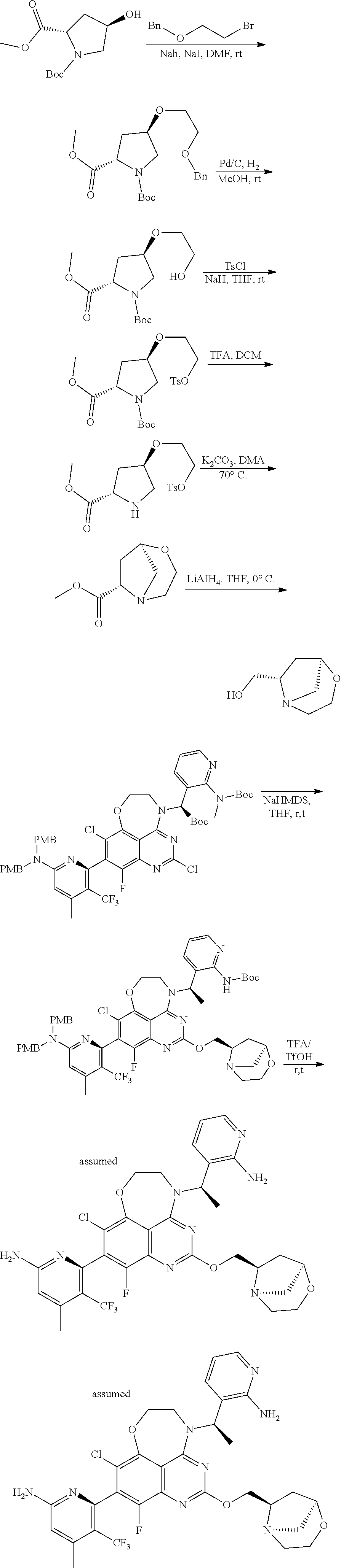

Step 1: 1-(tert-butyl) 2-methyl (2S,4R)-4-(2-(benzyloxy)ethoxy)pyrrolidine-1,2-dicarboxylate

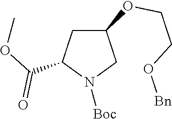 [SMILES: COC(=O)[C@@H]1C[C@@H](OCCOCc2ccccc2)CN1C(=O)OC(C)(C)C]

To a solution of 1-(tert-butyl) 2-methyl (2S,4R)-4-hydroxypyrrolidine-1,2-dicarboxylate (30.0 g, 122.31 mmol) in N,N-dimethylformamide (300 mL) was added sodium hydride (5.9 g, 147.5 mmol, 60% dispersion in mineral oil), the mixture was stirred at 0° C. for 10 minutes, then sodium iodide (1.83 g, 12.23 mmol) and ((2-bromoethoxy)methyl)benzene (26.3 g, 122.27 mmol) was added into the mixture and stirred at 25° C. for 1 h. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (76:24) to afford 1-(tert-butyl) 2-methyl (2S,4R)-4-(2-(benzyloxy)ethoxy)pyrrolidine-1,2-dicarboxylate (17.10 g, 45.06 mmol, 36.8% yield) as a colorless oil. LC-MS: (ESI, m/z): 380.2 [M+H]⁺

Step 2: 1-(tert-butyl) 2-methyl (2S,4R)-4-(2-hydroxyethoxy)pyrrolidine-1,2-dicarboxylate

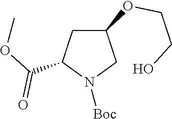 [SMILES: COC(=O)[C@@H]1C[C@@H](OCCO)CN1C(=O)OC(C)(C)C]

To a solution of 1-(tert-butyl) 2-methyl (2S,4R)-4-(2-(benzyloxy)ethoxy)pyrrolidine-1,2-dicarboxylate (17.1 g, 44.8 mmol) in methyl alcohol (170 mL) was added palladium on active carbon (9.5 g, 89.27 mmol), the mixture was stirred under atmospheric pressure of hydrogen at room temperature 15 hour. After completion, the solids were filtered out. After filtration, the filtrate was concentrated under reduced pressure to afford 1-(tert-butyl) 2-methyl (2S,4R)-4-(2-hydroxyethoxy)pyrrolidine-1,2-dicarboxylate (12.8 g, 44.24 mmol, 98.3% yield) as a colorless oil. LC-MS: (ESI, m/z): 290.2 [M+H]⁺

Step 3: 1-(tert-butyl) 2-methyl (2S,4R)-4-(2-(tosyloxy)ethoxy)pyrrolidine-1,2-dicarboxylate

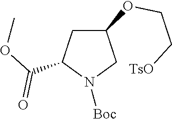 [SMILES: COC(=O)[C@@H]1C[C@@H](OCCOS(=O)(=O)c2ccc(C)cc2)CN1C(=O)OC(C)(C)C]

To a solution of 1-(tert-butyl) 2-methyl (2S,4R)-4-(2-hydroxyethoxy)pyrrolidine-1,2-dicarboxylate (13.6 g, 47.01 mmol) in tetrahydrofuran (128 mL) was added sodium hydride (5.6 g, 140 mmol, 60% dispersion in mineral oil), the mixture was stirred at 0° C. for 10 minutes. Then 4-methylbenzenesulfonyl chloride (18.00 g, 94.41 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product was used in the next step without purification. The residue was purified by flash chromatography on silica gel eluting with petroleum ether/ethyl acetate (1:1) to afford 1-(tert-butyl) 2-methyl (2S,4R)-4-(2-(tosyloxy)ethoxy)pyrrolidine-1,2-dicarboxylate (9.4 g, 18.86 mmol, 40.1% yield) as a colorless oil. LC-MS: (ESI, m/z): 444.2 [M+H]⁺

Step 4: methyl (2S,4R)-4-(2-(tosyloxy)ethoxy)pyrrolidine-2-carboxylate

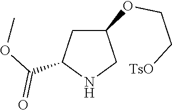 [SMILES: COC(=O)[C@@H]1C[C@@H](OCCOS(=O)(=O)c2ccc(C)cc2)CN1]

A solution of 1-(tert-butyl) 2-methyl (2S,4R)-4-(2-(tosyloxy)ethoxy)pyrrolidine-1,2-dicarboxylate (9.30 g, 20.97 mmol) in 2,2,2-trifluoroacetic acid (20 mL) and dichloromethane (20 mL) was stirred at 25° C. for 20 minutes. After completion, the solvent was removed under vacuum. The crude product was used in the next step without purification. LC-MS: (ESI, m/z): 344.1 [M+H]⁺

Step 5: methyl (5R,7S)-4-oxa-1-azabicyclo[3.2.1]octane-7-carboxylate

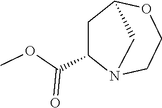

A solution of methyl (2S,4R)-4-(2-(tosyloxy)ethoxy)pyrrolidine-2-carboxylate (6.40 g, 18.64 mmol) and potassium carbonate (15.54 g, 112.63 mmol) in N,N-dimethylacetamide (15 mL) was stirred at 70° C. for 1 h. After completion, the solids were filtered out. The resulting residue was purified by reverse phase chromatography (acetonitrile/0.1% NH₄HCO₃ in water) to afford methyl (5R,7S)-4-oxa-1-azabicyclo[3.2.1]octane-7-carboxylate (450 mg, 2.63 mmol, 14.1% yield) as a colorless oil. LC-MS: (ESI, m/z): 172.1 [M+H]⁺

Step 6: ((5R,7S)-4-oxa-1-azabicyclo[3.2.1]octan-7-yl)methanol

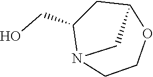

To a solution of methyl (5R,7S)-4-oxa-1-azabicyclo[3.2.1]octane-7-carboxylate (420.0 mg, 2.45 mmol) in tetrahydrofuran (5 mL) was added lithium aluminium hydride (280.0 mg, 7.37 mmol), the mixture was stirred at 0° C. for 0.5 hour. After completion, the reaction was quenched with sodium sulfate decahydrate. The solids were filtered out. After filtration, the filtrate was concentrated under reduced pressure to afford ((5R,7S)-4-oxa-1-azabicyclo[3.2.1]octan-7-yl)methanol (160 mg, 1.12 mmol, 45.5% yield) as a colorless oil. LC-MS: (ESI, m/z): 144.1 [M+H]⁺

Step 7: tert-butyl (3-((1R)-1-(2-(((5R,7S)-4-oxa-1-azabicyclo[3.2.1]octan-7-yl)methoxy)-9-((R)-6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate

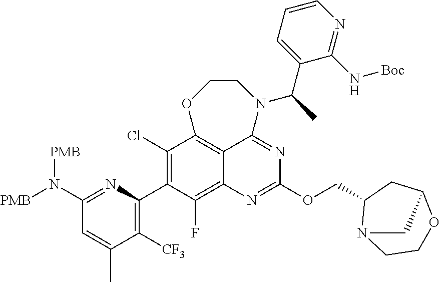

Under nitrogen, to a solution of ((5R,7S)-4-oxa-1-azabicyclo[3.2.1]octan-7-yl)methanol (260.0 mg, 1.82 mmol) in tetrahydrofuran (9 mL) was added sodium bis(trimethylsilyl)amide (2.7 mL, 2.7 mmol, 1M in tetrahydrofuran), the mixture was stirred at 25° C. for 10 minutes. Then tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]-N-tert-butoxycarbonyl-carbamate (desired atropisomer) (900.0 mg, 0.89 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (96:4) to afford tert-butyl (3-((1R)-1-(2-(((5R,7S)-4-oxa-1-azabicyclo[3.2.1]octan-7-yl)methoxy)-9-((R)-6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate (500 mg, 0.45 mmol, 50.2% yield). LC-MS: (ESI, m/z): 1015.4 [M+H]⁺.

Step 8: 6-((R)-2-(((1R,5R,7S)-4-oxa-1-azabicyclo[3.2.1]octan-7-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine & 6-((R)-2-(((1R,5R,7R)-4-oxa-1-azabicyclo[3.2.1]octan-7-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

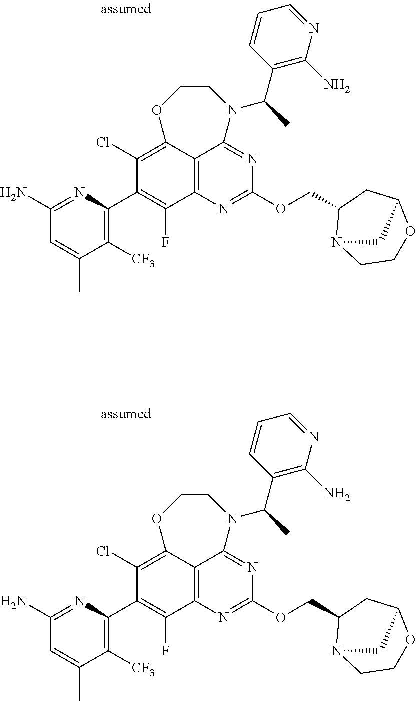

A solution of tert-butyl (3-((1R)-1-(2-(((5R,7S)-4-oxa-1-azabicyclo[3.2.1]octan-7-yl)methoxy)-9-((R)-6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate (480.0 mg, 0.43 mmol) in trifluoroacetic acid (4 mL) and trifuoromethanesulfonic acid (0.4 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 19*250 mm, 5 μm; Mobile Phase A: Water (10 mmol/L NH₄HCOs), Mobile Phase B: MeOH-HPLC; Flow rate: 25 mL/min; Gradient: 20% B to 50% B in 7 min, 50% B; Wave Length: 254/220 nm; RT1(min): 5.5 to afford 6-((R)-2-(((1R,5R,7S)-4-oxa-1-azabicyclo[3.2.1]octan-7-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (16.9 mg, 0.03 mmol, 5.8% yield) and 6-((R)-2-(((1R,5R,7R)-4-oxa-1-azabicyclo[3.2.1]octan-7-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (5.8 mg, 0.01 mmol, 2% yield). The stereo chemistry of title compounds was arbitrarily assigned.

Example 92a: LC-MS: (ESI, m/z): 675.3 [M+H]⁺, ¹H NMR (300 MHz, DMSO-d₆, ppm) δ 7.95 (dd, J=4.9, 1.7 Hz, 1H), 7.61 (dd, J=7.5, 1.8 Hz, 1H), 6.80 (s, 2H), 6.65 (dd, J=7.5, 4.9 Hz, 1H), 6.46 (s, 1H), 6.24 (q, J=6.8 Hz, 1H), 5.65 (s, 2H), 4.41 (dd, J=12.0, 6.1 Hz, 1H), 4.35-4.16 (m, 3H), 4.02 (dd, J=10.8, 6.5 Hz, 1H), 3.86-3.71 (m, 1H), 3.68-3.53 (m, 2H), 3.45-3.35 (m, 3H), 3.00-2.90 (m, 1H), 2.88-2.80 (m, 1H), 2.58-2.52 (m, 1H), 2.34 (d, J=1.2 Hz, 3H), 2.27-2.15 (m, 1H), 1.72-1.58 (m, 1H), 1.55 (d, J=6.8 Hz, 3H). LC-MS: (ESI, m/z): 675.3 [M+H]⁺

Example 92b: LC-MS: (ESI, m/z): 675.3 [M+H]⁺, ¹H NMR (300 MHz, Methanol-d₈, ppm) δ 7.96 (dd, J=5.1, 1.7 Hz, 1H), 7.80 (dd, J=7.6, 1.4 Hz, 1H), 6.79 (dd, J=7.6, 5.1 Hz, 1H), 6.59 (s, 1H), 5.95 (q, J=7.0 Hz, 1H), 5.70 (q, J=5.9 Hz, 1H), 4.54 (dd, J=11.5, 7.8 Hz, 1H), 4.46-4.31 (m, 2H), 4.10-3.86 (m, 2H), 3.66 (dd, J=12.6, 5.3 Hz, 1H), 3.29-3.18 (m, 1H), 3.17-3.09 (m, 1H), 3.00-2.90 (m, 1H), 2.83 (dd, J=13.3, 3.9 Hz, 1H), 2.51-2.38 (m, 4H), 2.00-1.87 (m, 1H), 1.77 (d, J=7.1 Hz, 3H), 1.57 (d, J=6.0 Hz, 3H).

Example 93a & 93b & 93c & 93d: 6-((R)-4-((R)-1-(2-aminopyridin-3-yl)ethyl)-8-chloro-10-fluoro-2-(((3S,6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((3S,6R,8aR)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((3R,6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((3R,6R,8aR)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

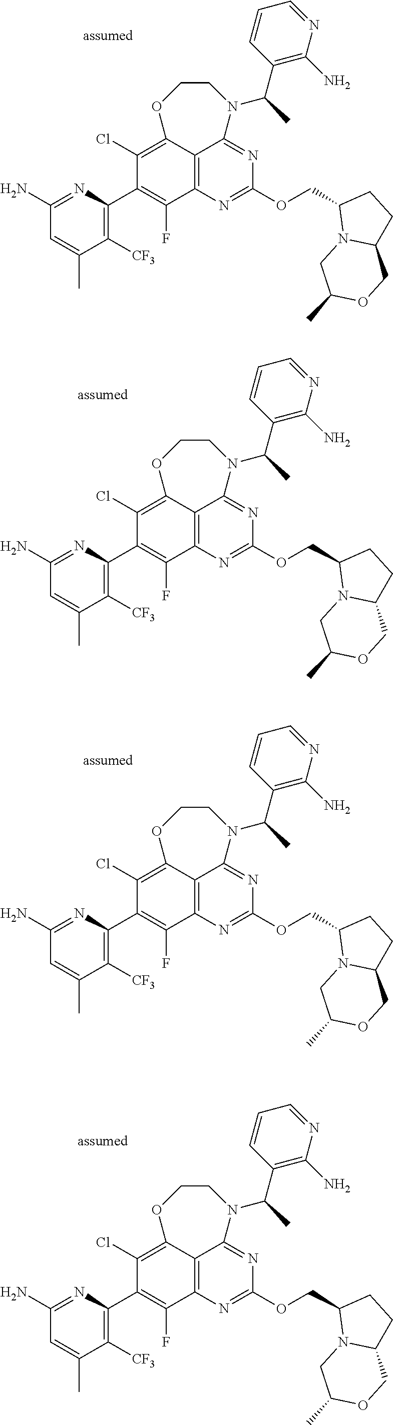

### Synthetic Route

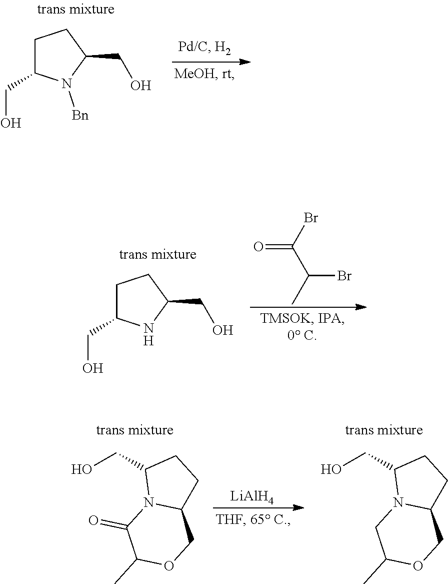

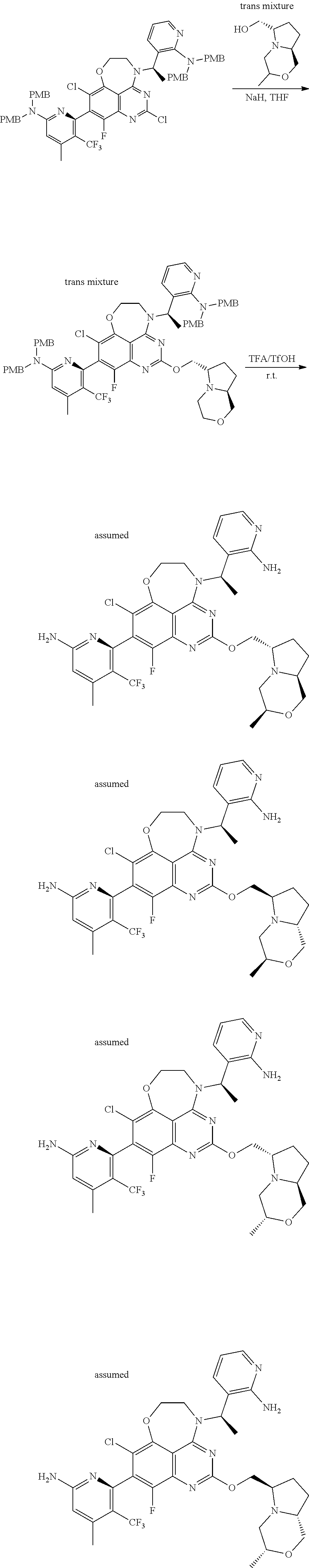

Step 1: ((2S,5S)-pyrrolidine-2,5-diyl)dimethanol (Trans Mixture)

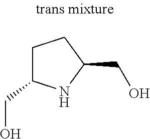

A mixture of ((2S,5S)-1-benzylpyrrolidine-2,5-diyl)dimethanol (trans mixture) (4.0 g, 18.08 mmol) and Palladium carbon (2.0 g, 1.89 mmol) in methyl alcohol (60 mL) was stirred at room temperature for 1 hour under hydrogen. The solids were filtered out. After filtration, the filtrate was concentrated under reduced pressure. The crude product was used in the next step without purification. LC-MS: (ESI, m/z): [M+H]⁺=132.1.

Step 2: (6S,8aS)-6-(hydroxymethyl)-3-methyltetrahydro-1H-pyrrolo[2,1-c][1,4]oxazin-4(3H)-one (Trans Mixture)

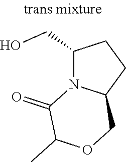

To a solution of ((2S,5S)-pyrrolidine-2,5-diyl)dimethanol (trans mixture) (2.30 g, 17.53 mmol) in 2-propanol (23 mL) was added potassium trimethylsilanolate (4.5 g, 35.07 mmol) and 2-bromopropanoyl bromide (4.2 g, 19.29 mmol), the mixture was stirred at 0° C. to room temperature for 16 hours. After completion, the solvent was removed under vacuum. The residue was purified by flash chromatography on silica gel eluting with dichloromethane/methanol (96:4) to afford (6S,8aS)-6-(hydroxymethyl)-3-methyltetrahydro-1H-pyrrolo[2,1-c][1,4]oxazin-4(3H)-one (trans mixture) (900 mg, 4.86 mmol, 27.7% yield) as a colorless clear oil. LC-MS: (ESI, m/z): 186.1 [M+H]⁺

Step 3: ((6S,8aS)-3-methylhexahydro-1H-pyrrolo[2,1-c][1,4]oxazin-6-yl)methanol (Trans Mixture)

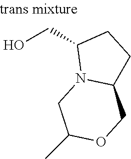

To a solution of (6S,8aS)-6-(hydroxymethyl)-3-methyltetrahydro-1H-pyrrolo[2,1-c][1,4]oxazin-4(3H)-one (trans mixture) (1.2 g, 6.48 mmol) in tetrahydrofuran (12 mL) was added lithium aluminium hydride (1.5 g, 38.87 mmol) was stirred at 65° C. for 1 hour. The reaction was quenched with sodium sulfate decahydrate. The solids were filtered out. After filtration, the filtrate was concentrated under reduced pressure to afford ((6S,8aS)-3-methylhexahydro-1H-pyrrolo[2,1-c][1,4]oxazin-6-yl)methanol (trans mixture) (900 mg, 5.26 mmol, 81.1% yield) as a colorless oil. LC-MS: (ESI, m/z): 172.1 [M+H]⁺

Step 4: 6-((9R)-4-((R)-1-(2-(bis(4-methoxybenzyl)amino)pyridin-3-yl)ethyl)-8-chloro-10-fluoro-2-(((6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (trans mixture)

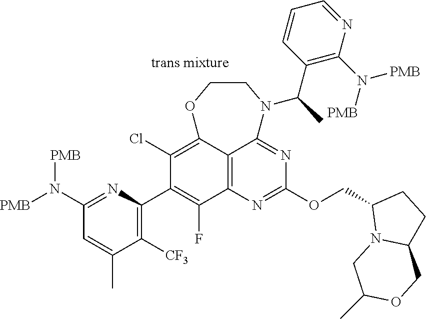

To a solution of ((6S,8aS)-3-methylhexahydro-1H-pyrrolo[2,1-c][1,4]oxazin-6-yl)methanol (trans mixture) (588.0 mg, 3.43 mmol) in tetrahydrofuran (12 mL) was added sodium hydride (180.0 mg, 4.5 mmol, 60% dispersion in mineral oil), the mixture was stirred at 0° C. for 10 minutes. Then 6-((R)-4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (1.20 g, 1.14 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (96:4) to afford 6-((9R)-4-((R)-1-(2-(bis(4-methoxybenzyl)amino)pyridin-3-yl)ethyl)-8-chloro-10-fluoro-2-(((6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (trans mixture) (1.02 g, 1.00 mmol, 87.5% yield) as a yellow solid.

Step 5: 6-((R)-4-((R)-1-(2-aminopyridin-3-yl)ethyl)-8-chloro-10-fluoro-2-(((3S,6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((3S,6R,8aR)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((3R,6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((3R,6R,8aR)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

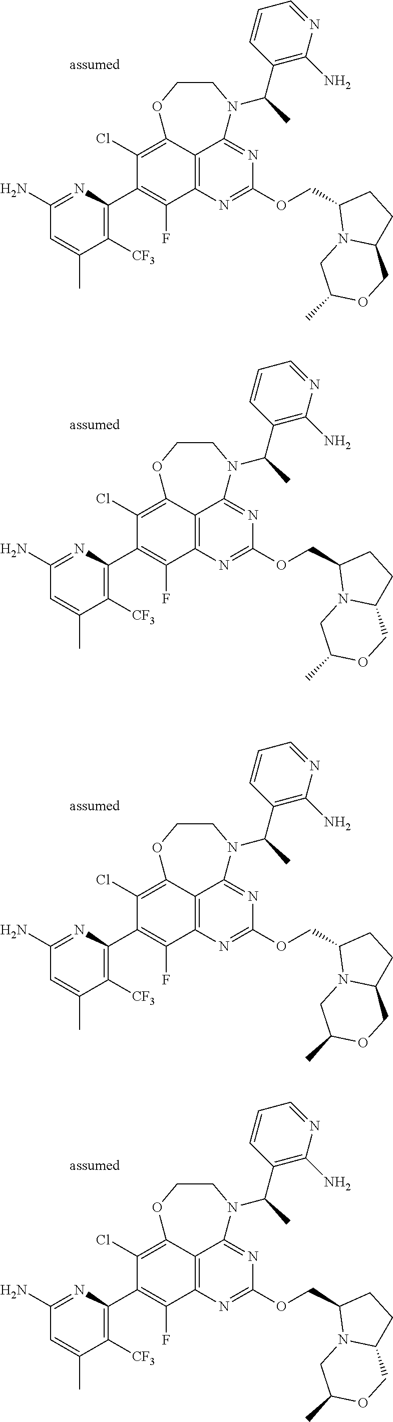

A solution of 6-((9R)-4-((R)-1-(2-(bis(4-methoxybenzyl)amino)pyridin-3-yl)ethyl)-8-chloro-10-fluoro-2-(((6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (trans mixture) (600.0 mg, 0.51 mmol) in trifluoroacetic acid (5 mL) and trifuoromethanesulfonic acid (0.5 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9 to afford two diastereoisomers. The first diastereoisomer was isolated by Prep-Chiral-HPLC with the following conditions: Column: CHIRALPAK IF, 2*25 cm, 5 μm; Mobile Phase A: MtBE (0.5% 2M NH₃-MeOH)-HPLC, Mobile Phase B: MeOH:DCM=1:1-HPLC; Flow rate: 20 mL/min; Gradient: 5% B to 5% B in 22 min; Wave Length: 220/254 nm; RT1(min): 8.885; RT2(min): 11.376; Sample Solvent: EtOH-HPLC; Injection Volume: 0.6 mL; Number Of Runs: 9 to afford 6-((R)-4-((R)-1-(2-aminopyridin-3-yl)ethyl)-8-chloro-10-fluoro-2-(((3S,6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (19.6 mg, 0.03 mmol, 5.3% yield) and 6-((R)-4-((R)-1-(2-aminopyridin-3-yl)ethyl)-8-chloro-10-fluoro-2-(((3S,6R,8aR)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (19.8 mg, 0.03 mmol, 5.4% yield). The second diastereoisomer was isolated by Prep-Chiral-HPLC with the following conditions: Column: CHIRALPAK IE-3, 4.6*50 mm, 3 um; Mobile Phase A: Hex:DCM=3:1) (0.1% DEA) EtOH=80:20; Flow rate: 1 mL/min; Gradient: 0% B to 0% B; Injection Volume: 5 ul mL to afford 6-((R)-4-((R)-1-(2-aminopyridin-3-yl)ethyl)-8-chloro-10-fluoro-2-(((3R,6S,8aS)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (48 mg, 0.07 mmol, 13.5% yield) and 6-((R)-4-((R)-1-(2-aminopyridin-3-yl)ethyl)-8-chloro-10-fluoro-2-(((3R,6R,8aR)-3-methylhexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (49.1 mg, 0.07 mmol, 13.7% yield). The stereo chemistry of title compounds was arbitrarily assigned.

Example 93a: ¹H NMR (300 MHz, DMSO-d₆, ppm) δ 7.98 (dd, J=4.9, 1.7 Hz, 1H), 7.64 (dd, J=7.5, 1.8 Hz, 1H), 6.80 (s, 2H), 6.67 (dd, J=7.5, 4.9 Hz, 1H), 6.48 (s, 1H), 6.24 (q, J=6.7 Hz, 1H), 5.68 (s, 2H), 4.42 (dd, J=10.7, 5.0 Hz, 2H), 4.33-4.21 (m, 1H), 4.14 (dd, J=10.8, 6.2 Hz, 1H), 3.68-3.48 (m, 4H), 3.42-3.33 (m, 1H), 3.15 (t, J=10.9 Hz, 1H), 3.06-2.87 (m, 2H), 2.49-2.41 (m, 1H), 2.37 (d, J=2.2 Hz, 3H), 2.17-1.96 (m, 1H), 1.88-1.62 (m, 2H), 1.57 (d, J=6.8 Hz, 3H), 1.37-1.19 (m, 1H), 0.94 (d, J=6.2 Hz, 3H). LC-MS: (ESI, m/z): 703.2 [M+H]⁺, Chiral HPLC: Column: CHIRALPAK ID-3, 4.6*50 mm, 3 um; detected at 254 nm; (Hex:DCM=3:1)(0.1% DEA):IPA=80:20; Flow rate: 1 mL/min; Retention time: 1.716 min (faster peak)

Example 93b: ¹H NMR (300 MHz, DMSO-d₆, ppm) δ 7.97 (dd, J=4.9, 1.7 Hz, 1H), 7.63 (dd, J=7.5, 1.8 Hz, 1H), 6.80 (s, 2H), 6.67 (dd, J=7.5, 4.9 Hz, 1H), 6.48 (s, 1H), 6.26 (q, J=6.7 Hz, 1H), 5.70 (s, 2H), 4.51-4.36 (m, 2H), 4.32-4.20 (m, 1H), 4.11 (dd, J=10.8, 6.1 Hz, 1H), 3.74-3.44 (m, 4H), 3.44-3.36 (m, 1H), 3.16 (t, J=10.9 Hz, 1H), 3.09-2.91 (m, 2H), 2.63-2.53 (m, 1H), 2.37 (d, J=2.3 Hz, 3H), 2.18-2.01 (m, 1H), 1.88-1.71 (m, 1H), 1.71-1.60 (m, 1H), 1.57 (d, J=6.8 Hz, 3H), 1.37-1.19 (m, 1H), 0.97 (d, J=6.2 Hz, 3H). LC-MS: (ESI, m/z): 703.2 [M+H]⁺, Chiral HPLC: Column: CHIRALPAK ID-3, 4.6*50 mm, 3 um; detected at 254 nm; (Hex:DCM=3:1)(0.1% DEA):IPA=80:20; Flow rate: 1 mL/min; Retention time: 2.023 min (slower peak).

Example 93c: ¹H NMR (300 MHz, DMSO-d₆, ppm) δ 7.95 (dd, J=4.9, 1.7 Hz, 1H), 7.60 (dd, J=7.5, 1.8 Hz, 1H), 6.79 (s, 2H), 6.65 (dd, J=7.5, 4.9 Hz, 1H), 6.46 (s, 1H), 6.22 (q, J=6.8 Hz, 1H), 5.66 (s, 2H), 4.41 (dd, J=12.2, 5.8 Hz, 1H), 4.33-4.17 (m, 2H), 4.05 (dd, J=10.8, 6.1 Hz, 1H), 3.73 (d, J=2.8 Hz, 2H), 3.67-3.59 (m, 1H), 3.56-3.43 (m, 1H), 3.42-3.32 (m, 1H), 3.27-3.14 (m, 1H), 2.98-2.81 (m, 1H), 2.65-2.53 (m, 1H), 2.35 (d, J=2.3 Hz, 3H), 2.29-2.04 (m, 2H), 1.88-1.70 (m, 1H), 1.68-1.42 (m, 5H), 1.03 (d, J=6.2 Hz, 3H). LC-MS: (ESI, m/z): 703.2 [M+H]⁺. Chiral HPLC: Column: CHIRAL Cellulose-SB, 4.6*100 mm, 3 μm; detected at 254 nm; Hex (0.1% DEA):EtOH=75:25; Flow rate: 1 mL/min; Retention time: 7.850 min (faster peak)

Example 93d: ¹H NMR (300 MHz, DMSO-d₈, ppm) δ 7.95 (dd, J=4.9, 1.7 Hz, 1H), 7.61 (dd, J=7.6, 1.8 Hz, 1H), 6.79 (s, 2H), 6.65 (dd, J=7.5, 4.9 Hz, 1H), 6.46 (s, 1H), 6.23 (d, J=6.8 Hz, 1H), 5.66 (s, 2H), 4.41 (dd, J=11.9, 6.1 Hz, 1H), 4.33-4.16 (m, 2H), 4.05 (dd, J=10.8, 6.1 Hz, 1H), 3.74 (d, J=2.8 Hz, 2H), 3.62 (dd, J=15.7, 6.8 Hz, 1H), 3.56-3.44 (m, 1H), 3.43-3.33 (m, 1H), 3.28-3.14 (m, 1H), 2.93-2.80 (m, 1H), 2.64-2.55 (m, 1H), 2.35 (d, J=2.3 Hz, 3H), 2.30-2.01 (m, 2H), 1.88-1.70 (m, 1H), 1.68-1.44 (m, 5H), 1.03 (d, J=6.2 Hz, 3H). LC-MS: (ESI, m/z): 703.2 [M+H]⁺. Chiral HPLC: Column: CHIRAL Cellulose-SB, 4.6*100 mm, 3 μm; detected at 254 nm; Hex (0.1% DEA):EtOH=75:25; Flow rate: 1 mL/min; Retention time: 9.376 min (slower peak)

Example 94: 6-((9R)-4-((R)-1-(2-aminopyridin-3-yl)ethyl)-8-chloro-10-fluoro-2-(2-(3-methyl-3,8-diazabicyclo[3.2.1]octan-8-yl)ethoxy)-5,6-dihydro-4H-[1,4]oxazepino[5,6,7-de]quinazolin-9-yl)-4-methyl-5-(trifluoromethyl)pyridin-2-amine

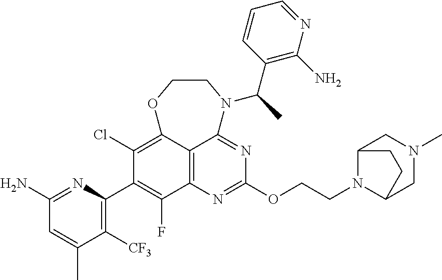

### Synthetic Route

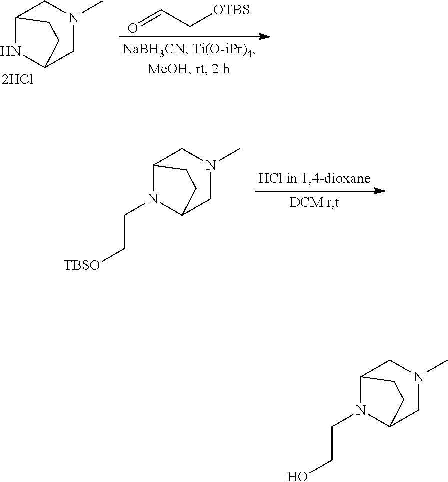

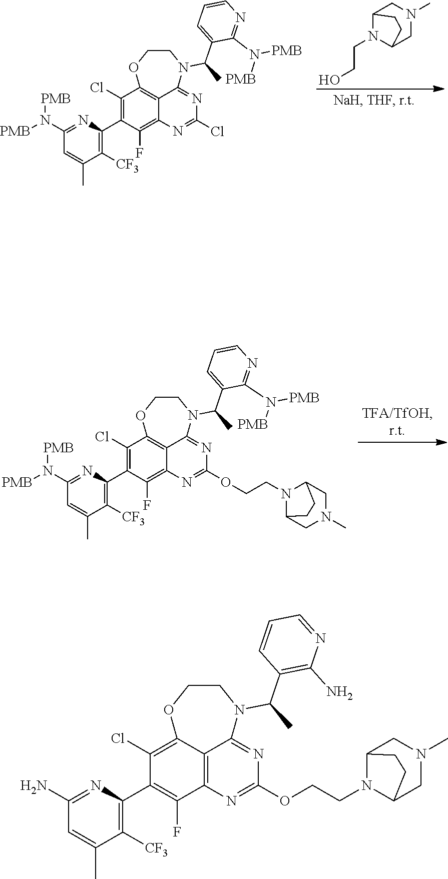

Step 1: 8-(2-((tert-butyldimethylsilyl)oxy)ethyl)-3-methyl-3,8-diazabicyclo[3.2.1]octane

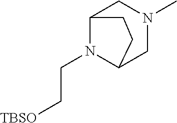 [SMILES: CN1CC2CCC(C1)N2CCO[Si](C)(C)C(C)(C)C]

A solution of 3-methyl-3,8-diazabicyclo[3.2.1]octane (1.20 g, 6.00 mmol), 2-((tert-butyldimethylsilyl)oxy)acetaldehyde (3.10 g, 18.1 mmol), sodium cyanoborohydride (1.1 g, 18.1 mmol) and titanium tetraisopropanolate (2.4 mL) in methyl alcohol (12 mL) was stirred at room temperature for 1 hour. The solvent was removed under vacuum. The residue was purified by flash chromatography on silica gel eluting with dichloromethane/methanol (96:4) to afford 88-(2-((tert-butyldimethylsilyl)oxy)ethyl)-3-methyl-3,8-diazabicyclo[3.2.1]octane (1.70 g, 5.98 mmol, 99.1% yield) as a colorless oil. LC-MS: (ESI, m/z): 285.2 [M+H]⁺

Step 2: 2-(3-methyl-3,8-diazabicyclo[3.2.1]octan-8-yl)ethan-1-ol

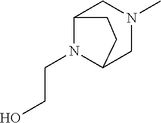

A solution of 8-(2-((tert-butyldimethylsilyl)oxy)ethyl)-3-methyl-3,8-diazabicyclo[3.2.1]octane (2.3 g, 8.1 mmol) in 4M hydrochloric acid in 1,4-dioxane (10 mL) and dichloromethane (10 mL) was stirred at room temperature for 20 mins. After completion, the solvent was removed under vacuum. The residue was purified by flash chromatography on silica gel eluting with dichloromethane/methanol (90:10) to afford 2-(3-methyl-3,8-diazabicyclo[3.2.1]octan-8-yl)ethan-1-ol (450 mg, 2.64 mmol, 32.7% yield) as a colorless oil. LC-MS: (ESI, m/z): 171.1 [M+H]⁺

Step 3: (6R)-6-(4-((R)-1-(2-(bis(4-methoxybenzyl)amino)pyridin-3-yl)ethyl)-8-chloro-10-fluoro-2-(2-(3-methyl-3,8-diazabicyclo[3.2.1]octan-8-yl)ethoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

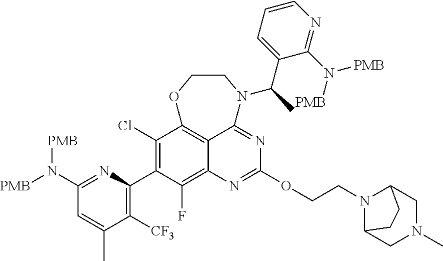

A solution of 2-(3-methyl-3,8-diazabicyclo[3.2.1]octan-8-yl)ethan-1-ol (100.0 mg, 0.59 mmol) and sodium hydride (31.0 mg, 0.78 mmol, 60% dispersion in mineral oil) in tetrahydrofuran (2 mL) was stirred at 0° C. for 10 minutes. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.19 mmol) was added and stirred at room temperature for 3 hours. After completion, the resulting solution was quenched with saturated ammonium chloride solution and extracted with ethyl acetate. The organic layers were combined, washed with brine, dried over anhydrous sodium sulfate and concentrated under vacuum. The residue was purified by flash chromatography on silica gel eluting with dichloromethane/methanol (96:4) to afford (6R)-6-(4-((R)-1-(2-(bis(4-methoxybenzyl)amino)pyridin-3-yl)ethyl)-8-chloro-10-fluoro-2-(2-(3-methyl-3,8-diazabicyclo[3.2.1]octan-8-yl)ethoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (190 mg, 0.16 mmol, 84.1% yield) as a yellow solid. LC-MS: (ESI, m/z): 171.0 [M+H]⁺

Step 4: 6-((9R)-4-((R)-1-(2-aminopyridin-3-yl)ethyl)-8-chloro-10-fluoro-2-(2-(3-methyl-3,8-diazabicyclo[3.2.1]octan-8-yl)ethoxy)-5,6-dihydro-4H-[1,4]oxazepino[5,6,7-de]quinazolin-9-yl)-4-methyl-5-(trifluoromethyl)pyridin-2-amine

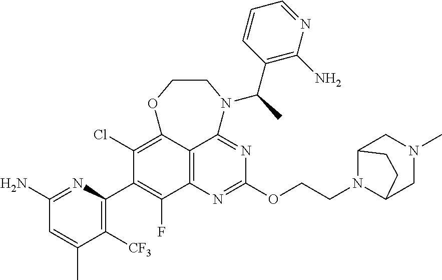

A solution of (6R)-6-(4-((R)-1-(2-(bis(4-methoxybenzyl)amino)pyridin-3-yl)ethyl)-8-chloro-10-fluoro-2-(2-(3-methyl-3,8-diazabicyclo[3.2.1]octan-8-yl)ethoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (170.0 mg, 0.14 mmol) trifluoroacetic acid (2 mL) and trifuoromethanesulfonic acid (0.2 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 23% B to 48% B in 10 min, 48% B; Wave Length: 254/220 nm; RT1(min): 9.6; to afford 6-((9R)-4-((R)-1-(2-aminopyridin-3-yl)ethyl)-8-chloro-10-fluoro-2-(2-(3-methyl-3,8-diazabicyclo[3.2.1]octan-8-yl)ethoxy)-5,6-dihydro-4H-[1,4]oxazepino[5,6,7-de]quinazolin-9-yl)-4-methyl-5-(trifluoromethyl)pyridin-2-amine (59.1 mg, 0.08 mmol, 58.5% yield). LC-MS: (ESI, m/z): 702.4 [M+H]⁺

Example 94: ¹H NMR (300 MHz, DMSO-da, ppm) δ 7.97 (dd, J=4.9, 1.7 Hz, 1H), 7.63 (dd, J=7.4, 1.8 Hz, 1H), 6.80 (s, 2H), 6.67 (dd, J=7.5, 4.9 Hz, 1H), 6.48 (s, 1H), 6.25 (q, J=6.7 Hz, 1H), 5.66 (s, 2H), 4.48-4.35 (m, 3H), 4.31-4.18 (m, 1H), 3.74-3.49 (m, 2H), 3.32-3.12 (m, 2H), 2.66 (t, J=6.4 Hz, 2H), 2.49-2.41 (m, 2H), 2.36 (s, 3H), 2.11 (d, J=10.0 Hz, 2H), 2.07 (s, 3H), 1.84-1.70 (m, 2H), 1.70-1.59 (m, 2H), 1.56 (d, J=6.8 Hz, 3H).

Example 95a & 95b: 6-((R)-4-((R)-1-(2-aminopyridin-3-yl)ethyl)-8-chloro-10-fluoro-2-((S)-2-morpholinopropoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-((R)-2-morpholinopropoxy)-5,6-dihydro-4H-[1,4]oxazepino[5,6,7-de]quinazolin-9-yl)-4-methyl-5-(trifluoromethyl)pyridin-2-amine

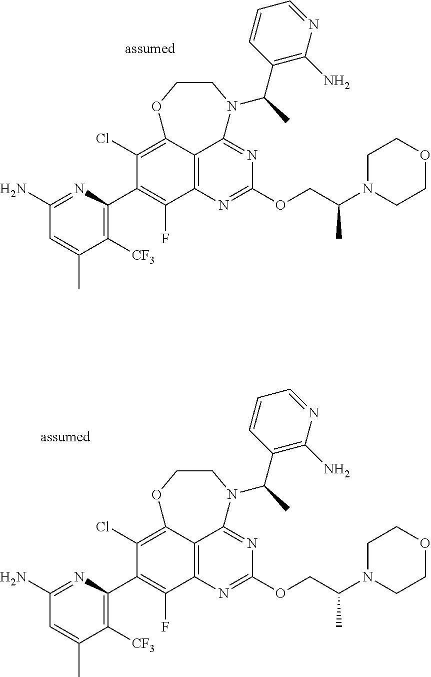

### Synthetic Route

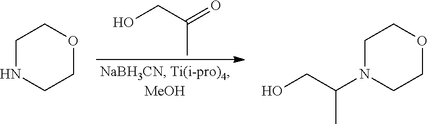

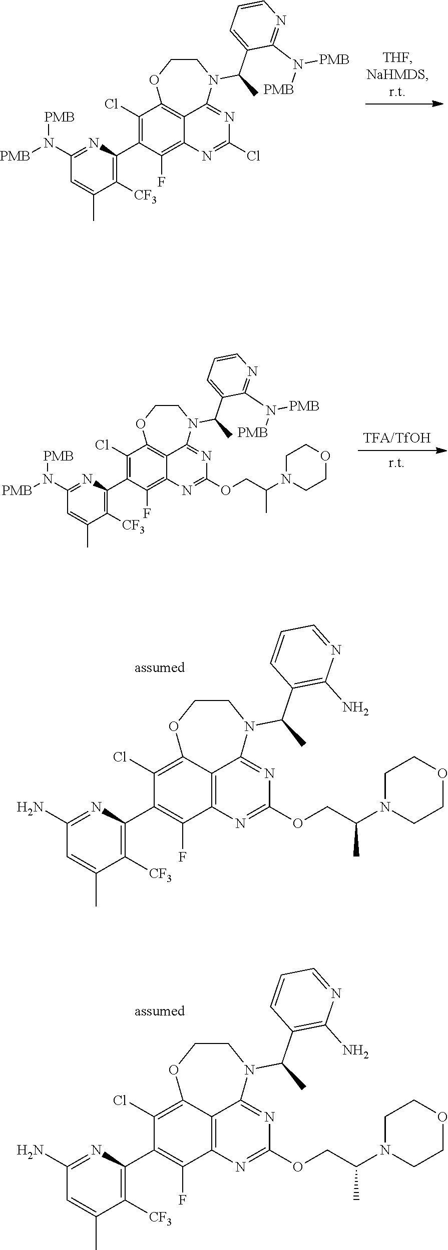

Step 1: 2-morpholinopropan-1-ol

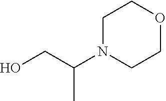

A mixture of morpholine (500 mg, 5.74 mmol) and 1-hydroxypropan-2-one (850.3 mg, 11.48 mmol) in tetrapropyl titanate (1.0 mL) and methyl alcohol (5.0 mL) was stirred at 25° C. for 0.5 hour. Then sodium cyanoborohydride (721.3 mg, 11.48 mmol) was added and stirred at 80° C. for 1 hour. After completion, the solvent was removed under vacuum. The residue was purified by flash chromatography on silica gel eluting with dichloromethanetmethanol (8:1) to afford 2-morpholinopropan-1-ol (500.0 mg, 3.44 mmol, 60% yield) as a yellow solid. LC-MS: (ESI, m/z): 146.1 [M+H]⁺.

Step 2: (6R)-6-(4-((R)-1-(2-(bis(4-methoxybenzyl)amino)pyridin-3-yl)ethyl)-8-chloro-10-fluoro-2-(2-morpholinopropoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

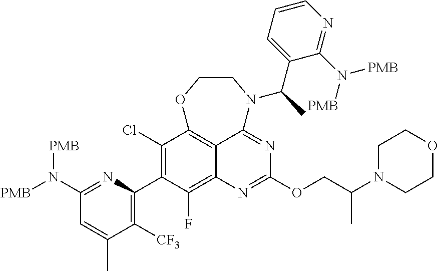

Under nitrogen, to a solution of 2-morpholinopropan-1-ol (276.8 mg, 1.91 mmol) in tetrahydrofuran (4 mL) was added sodium bis(trimethylsilyl)amide (2.28 mL, 2.28 mmol, 1 M in tetrahydrofuran), the mixture was stirred at 25° C. for 0.5 hour. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.0 mg, 0.38 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/ethyl acetate (1:5) to afford (6R)-6-(4-((R)-1-(2-(bis(4-methoxybenzyl)amino)pyridin-3-yl)ethyl)-8-chloro-10-fluoro-2-(2-morpholinopropoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (250.0 mg, 0.21 mmol, 56.6% yield) as a white solid. LC-MS: (ESI, m/z): 1157.5 [M+H]⁺.

Step 3: 6-((R)-4-((R)-1-(2-aminopyridin-3-yl)ethyl)-8-chloro-10-fluoro-2-((S)-2-morpholinopropoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-((R)-2-morpholinopropoxy)-5,6-dihydro-4H-[1,4]oxazepino[5,6,7-de]quinazolin-9-yl)-4-methyl-5-(trifluoromethyl)pyridin-2-amine

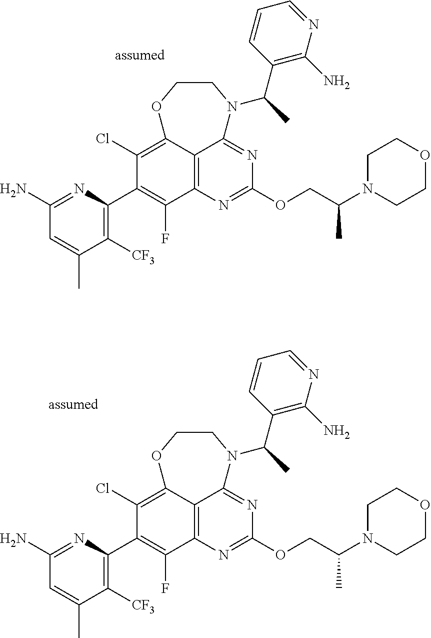

A solution of (6R)-6-(4-((R)-1-(2-(bis(4-methoxybenzyl)amino)pyridin-3-yl)ethyl)-8-chloro-10-fluoro-2-(2-morpholinopropoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (240.0 mg, 0.21 mmol) in trifluoroacetic acid (5 mL) and trifuoromethanesulfonic acid (0.5 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (8:1) to afford crude product. The crude product was purified by Prep-HPLC with the following conditions: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 27% B to 52% B in 9 min, 52%; 254/220 nm; RT1: 8.9 min to afford product. The product was further purified by Prep-CHIRAL-HPLC with the following conditions: CHIRALPAK IE, 2*25 cm, 5 μm; Mobile Phase A: Hex:Dichloromethane=3:1(0.5% 2M NH₃-Methanol)-HPLC, Mobile Phase B: Ethanol-HPLC; Flow rate: 20 mL/min; Gradient: 20% B to 20% B in 22 min; Wave Length: 220/254 nm; RT1: 16.244 min RT2(min): 19.107; Sample Solvent: EtOH; Injection Volume: 0.3 mL; Number Of Runs: 9 to afford 6-((R)-4-((R)-1-(2-aminopyridin-3-yl)ethyl)-8-chloro-10-fluoro-2-((S)-2-morpholinopropoxy)-5,6-dihydro-4H-[1,4]oxazepino[5,6,7-de]quinazolin-9-yl)-4-methyl-5-(trifluoromethyl)pyridin-2-amine (22.8 mg, 0.03 mmol, 16.2% yield) and 6-((R)-4-((R)-1-(2-aminopyridin-3-yl)ethyl)-8-chloro-10-fluoro-2-((R)-2-morpholinopropoxy)-5,6-dihydro-4H-[1,4]oxazepino[5,6,7-de]quinazolin-9-yl)-4-methyl-5-(trifluoromethyl)pyridin-2-amine (25.9 mg, 0.04 mmol, 18.4% yield). The stereo chemistry of title compounds was arbitrarily assigned.

Example 95a: ¹H NMR (300 MHz, DMSO-d₆, ppm) δ 7.97 (dd, J=4.9, 1.7 Hz, 1H), 7.74-7.55 (m, 1H), 6.81 (s, 2H), 6.67 (dd, J=7.5, 4.9 Hz, 1H), 6.48 (s, 1H), 6.26 (q, J=7.1 Hz, 1H), 5.71 (s, 2H), 4.53 (dd, J=11.1, 6.3 Hz, 1H), 4.49-4.38 (m, 1H), 4.35-4.13 (m, 2H), 3.65 (dd, J=15.5, 6.6 Hz, 1H), 3.57-3.45 (m, 4H), 3.45-3.33 (m, 1H), 3.03-2.88 (m, 1H), 2.60-2.53 (m, 4H), 2.37 (d, J=2.3 Hz, 3H), 1.57 (d, J=6.8 Hz, 3H), 1.06 (d, J=6.7 Hz, 3H). LC-MS: (ESI, m/z): 677.4 [M+H]⁺. Chiral HPLC: Column: CHIRALPAK IE-3, 4.6*50 mm, 3 um; detected at 254 nm; (Hex:DCM=3:1)(0.1% DEA):EtOH=70:30; Flow rate: 1 mL/min; Retention time: 2.981 min (faster peak).

Example 95b: ¹H NMR (300 MHz, DMSO-d₆, ppm) δ 7.97 (dd, J=4.9, 1.7 Hz, 1H), 7.63 (dd, J=7.5, 1.8 Hz, 1H), 6.81 (s, 2H), 6.67 (dd, J=7.5, 4.9 Hz, 1H), 6.48 (s, 1H), 6.39-6.14 (m, 1H), 5.69 (s, 2H), 4.60-4.37 (m, 2H), 4.34-4.08 (m, 2H), 3.65 (dd, J=15.7, 6.9 Hz, 1H), 3.61-3.47 (m, 4H), 3.44-3.35 (m, 1H), 3.09-2.86 (m, 1H), 2.62-2.53 (m, 4H), 2.37 (d, J=2.3 Hz, 3H), 1.57 (d, J=6.8 Hz, 3H), 1.08 (d, J=6.7 Hz, 3H). Chiral HPLC: Column: CHIRALPAK IE-3, 4.6*50 mm, 3 um; detected at 254 nm; (Hex:DCM=3:1)(0.1% DEA):EtOH=70:30; Flow rate: 1 mL/min; Retention time: 3.535 min (slower peak).

Example 96: 6-((R)-4-((R)-1-(2-aminopyridin-3-yl)ethyl)-8-chloro-10-fluoro-2-(((S)-morpholin-3-yl)methoxy)-5,6-dihydro-4H-[1,4]oxazepino[5,6,7-de]quinazolin-9-yl)-4-methyl-5-(trifluoromethyl)pyridin-2-amine

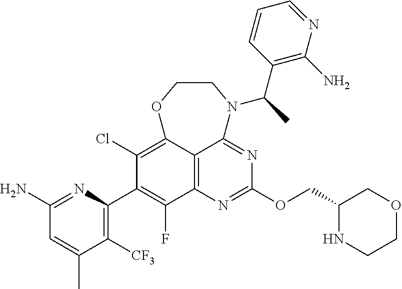

### Synthetic Route

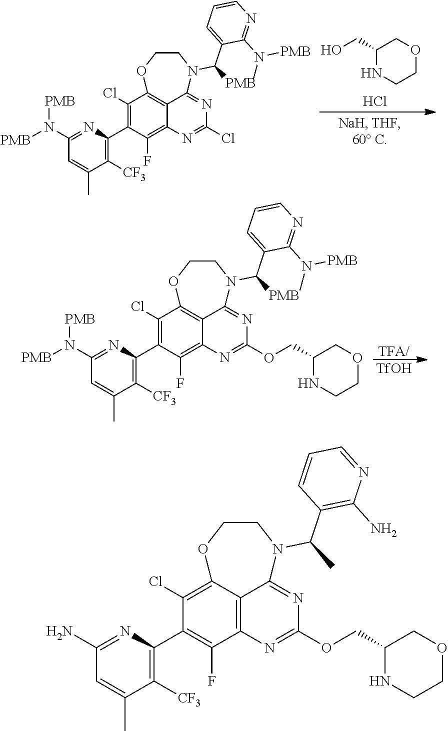

Step 1: (R)-6-(4-((R)-1-(2-(bis(4-methoxybenzyl)amino)pyridin-3-yl)ethyl)-8-chloro-10-fluoro-2-(((S)-morpholin-3-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

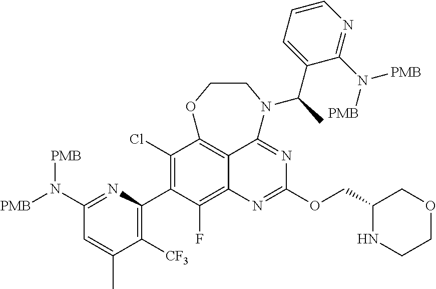

To a mixture of [(3R)-morpholin-3-yl]methanol hydrochloride (58.58 mg, 0.38 mmol) in tetrahydrofuran (3 mL) was added sodium hydride (45.76 mg, 1.14 mmol, 60% dispersion in mineral oil), the mixture was stirred for 10 min at 0° C. Then 6-[13-[(1R)-1-[2-[bis[(4-methoxyphenyl)methyl]amino]-3-pyridyl]ethyl]-3,8-dichloro-6-fluoro-10-oxa-2,4,13-triazatricyclo[7.4.1.05,14]tetradeca-1,3,5,7,9(14)-pentaen-7-yl]-N,N-bis[(4-methoxyphenyl)methyl]-4-methyl-5-(trifluoromethyl)pyridin-2-amine (200.0 mg, 0.19 mmol) in tetrahydrofuran (3 mL) was added, the mixture was stirred for 2 h at 60° C. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (95:5) to afford (R)-6-(4-((R)-1-(2-(bis(4-methoxybenzyl)amino)pyridin-3-yl)ethyl)-8-chloro-10-fluoro-2-(((S)-morpholin-3-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (120 mg, 0.11 mmol, 55.7% yield) as a yellow solid. LCMS (ESI, m/z): 1129.4 [M+H]⁺.

Step 2: 6-((R)-4-((R)-1-(2-aminopyridin-3-yl)ethyl)-8-chloro-10-fluoro-2-(((S)-morpholin-3-yl)methoxy)-5,6-dihydro-4H-[1,4]oxazepino[5,6,7-de]quinazolin-9-yl)-4-methyl-5-(trifluoromethyl)pyridin-2-amine

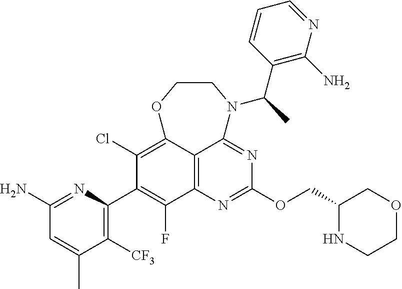

A mixture of (R)-6-(4-((R)-1-(2-(bis(4-methoxybenzyl)amino)pyridin-3-yl)ethyl)-8-chloro-10-fluoro-2-(((S)-morpholin-3-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (130.0 mg, 0.12 mmol) in trifluoroacetic acid (2 mL) and trifuoromethanesulfonic acid (0.2 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 29% B to 49% B in 8 min, 49% B; Wave Length: 254/220 nm; RT1(min): 8 to afford 6-((R)-4-((R)-1-(2-aminopyridin-3-yl)ethyl)-8-chloro-10-fluoro-2-(((S)-morpholin-3-yl)methoxy)-5,6-dihydro-4H-[1,4]oxazepino[5,6,7-de]quinazolin-9-yl)-4-methyl-5-(trifluoromethyl)pyridin-2-amine (10.8 mg, 0.0166 mmol, 14.5% yield). LCMS (ESI, m/z): 649.1 [M+H]⁺.

Example 96: ¹H NMR (300 MHz, DMSO-d₆, ppm) δ 7.97 (dd, J=4.9, 1.7 Hz, 1H), 7.63 (dd, J=7.6, 1.8 Hz, 1H), 6.82 (s, 2H), 6.66 (dd, J=7.5, 4.9 Hz, 1H), 6.47 (s, 1H), 6.24 (q, J=6.8 Hz, 1H), 5.69 (s, 2H), 4.51-4.34 (m, 1H), 4.28-4.12 (m, 3H), 3.81 (dd, J=10.7, 2.9 Hz, 1H), 3.68-3.55 (m, 2H), 3.44-3.34 (m, 2H), 3.28-3.18 (m, 1H), 3.14-3.00 (m, 1H), 2.85-2.68 (m, 2H), 2.64-2.55 (m, 1H), 2.36 (s, 3H), 1.56 (d, J=6.8 Hz, 3H).

Example 97: 6-((R)-4-((R)-1-(2-aminopyridin-3-yl)ethyl)-8-chloro-10-fluoro-2-(((R)-morpholin-3-yl)methoxy)-5,6-dihydro-4H-[1,4]oxazepino[5,6,7-de]quinazolin-9-yl)-4-methyl-5-(trifluoromethyl)pyridin-2-amine

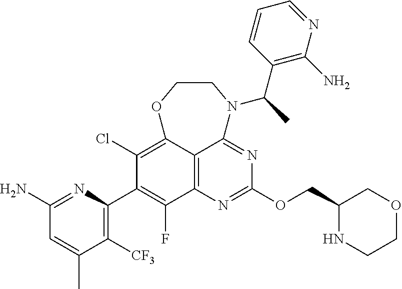

### Synthetic Route

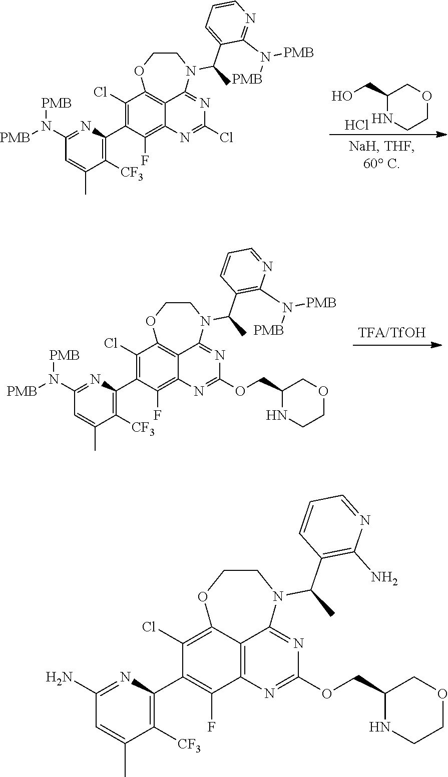

Step 1: (R)-6-(4-((R)-1-(2-(bis(4-methoxybenzyl)amino)pyridin-3-yl)ethyl)-8-chloro-10-fluoro-2-(((R)-morpholin-3-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

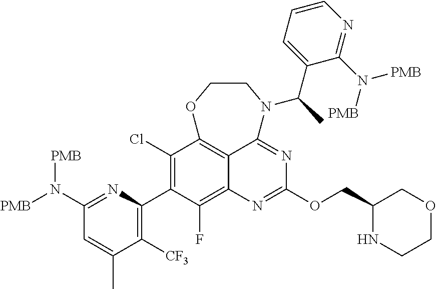

To a mixture of [(3S)-morpholin-3-yl]methanol hydrochloride (58.6 mg, 0.38 mmol) in tetrahydrofuran (3 mL) was added sodium hydride (45.8 mg, 1.14 mmol, 60% dispersion in mineral oil), the mixture was stirred for 10 min at 0° C. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.19 mmol) in tetrahydrofuran (3 mL) was added, the mixture was stirred for 2 hours at 60° C. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (95:5) to afford (R)-6-(4-((R)-1-(2-(bis(4-methoxybenzyl)amino)pyridin-3-yl)ethyl)-8-chloro-10-fluoro-2-(((R)-morpholin-3-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (130 mg, 0.12 mmol, 60.4% yield). LCMS (ESI, m/z): 1129.4 [M+H]⁺.

Step 2: 6-((R)-4-((R)-1-(2-aminopyridin-3-yl)ethyl)-8-chloro-10-fluoro-2-(((R)-morpholin-3-yl)methoxy)-5,6-dihydro-4H-[1,4]oxazepino[5,6,7-de]quinazolin-9-yl)-4-methyl-5-(trifluoromethyl)pyridin-2-amine

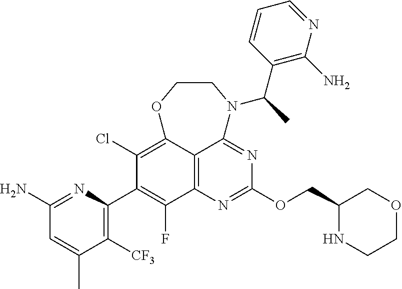

A mixture of (R)-6-(4-((R)-1-(2-(bis(4-methoxybenzyl)amino)pyridin-3-yl)ethyl)-8-chloro-10-fluoro-2-(((R)-morpholin-3-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (130 mg, 0.12 mmol) in trifluoroacetic acid (3 mL) and trifuoromethanesulfonic acid (0.3 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Shield RP18 OBD Column, 30*150 mm, 5 μm; Mobile Phase A: Water (10 mmol/L NH₄HCO₃), Mobile Phase B: ACN; Flow rate: 60 mL/min; Gradient: 29% B to 49% B in 8 min, 49% B; Wave Length: 254/220 nm; RT1(min): 8 to afford 6-((R)-4-((R)-1-(2-aminopyridin-3-yl)ethyl)-8-chloro-10-fluoro-2-(((R)-morpholin-3-yl)methoxy)-5,6-dihydro-4H-[1,4]oxazepino[5,6,7-de]quinazolin-9-yl)-4-methyl-5-(trifluoromethyl)pyridin-2-amine (15.3 mg, 0.02 mmol, 13.3% yield). LCMS (ESI, m/z): 649.3 [M+H]⁺.

Example 97: ¹H NMR (400 MHz, Methanol-d₄, ppm) δ 7.96 (dd, J=5.1, 1.6 Hz, 1H), 7.75 (d, 1H), 6.78 (dd, J=7.5, 5.1 Hz, 1H), 6.58 (s, 1H), 6.50 (q, J=6.8 Hz, 1H), 4.51-4.32 (m, 3H), 4.32-4.18 (m, 1H), 3.95 (dd, J=11.3, 3.1 Hz, 1H), 3.88-3.71 (m, 1H), 3.71-3.61 (m, 1H), 3.58-3.41 (m, 3H), 3.29-3.20 (m, 1H), 2.95-2.87 (m, 2H), 2.43 (s, 3H), 1.65 (d, J=6.9 Hz, 3H).

Example 98a & 98b: 6-((R)-4-((R)-1-(2-aminopyridin-3-yl)ethyl)-8-chloro-2-(((2R,6R)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((9R)-4-((R)-1-(2-aminopyridin-3-yl)ethyl)-8-chloro-2-(((2R,6S)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine

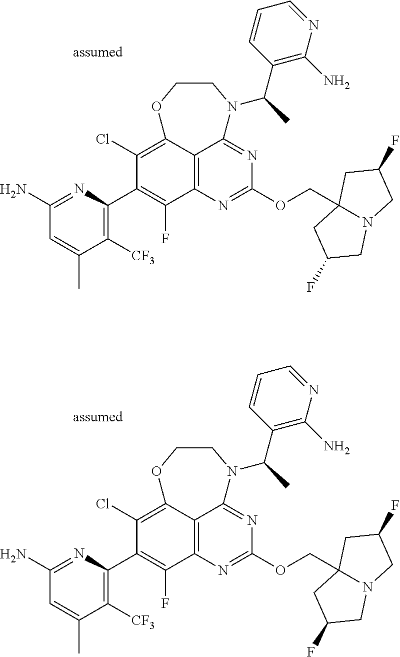

### Synthetic Route

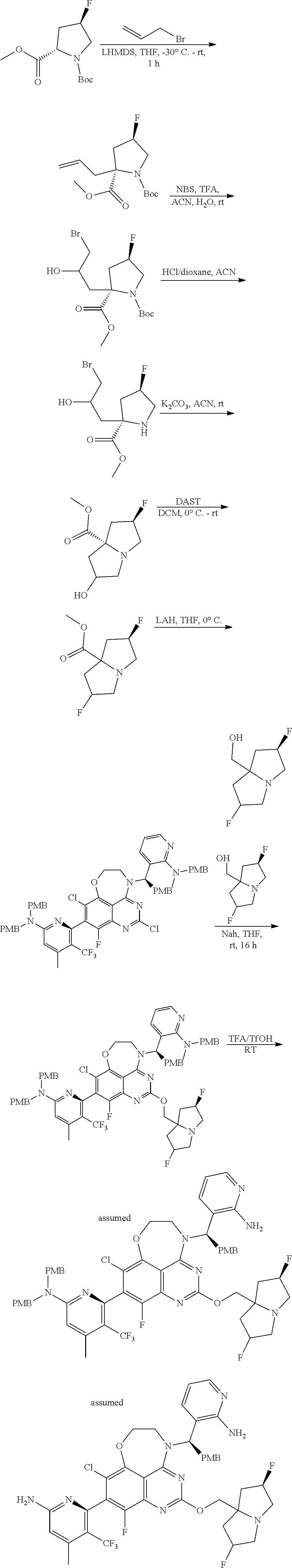

Step 1: 1-(tert-butyl) 2-methyl (2S,4R)-2-allyl-4-fluoropyrrolidine-1,2-dicarboxylate

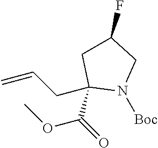 [SMILES: C=CC[C@@]1(C(=O)OC)C[C@@H](F)CN1C(=O)OC(C)(C)C]

To a solution of 1-(tert-butyl) 2-methyl (2S,4R)-4-fluoropyrrolidine-1,2-dicarboxylate (20.0 g, 80.89 mmol) in tetrahydrofuran (20 mL) was added lithium bis(trimethylsilyl)amide (161.77 mL, 161.77 mmol, 1M in tetrahydrofuran), the mixture was stirred at −78° C. for 3 min, then allyl bromide (14 mL, 161.77 mmol) was added and stirred at room temperature for 30 min. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by reverse phase chromatography (acetonitrile 0-60/0.1% NH₄HCO₃ in water) to afford 1-(tert-butyl) 2-methyl (2S,4R)-2-allyl-4-fluoropyrrolidine-1,2-dicarboxylate (25 g, 73.96 mmol, 91.4% yield). LCMS (ESI, m/z): 288.2 [M+H]⁺.

Step 2: 1-(tert-butyl) 2-methyl (2S,4R)-2-(3-bromo-2-hydroxypropyl)-4-fluoropyrrolidine-1,2-dicarboxylate

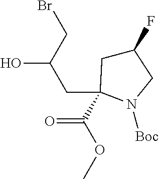 [SMILES: COC(=O)[C@]1(CC(O)CBr)C[C@@H](F)CN1C(=O)OC(C)(C)C]

To a solution of 1-(tert-butyl) 2-methyl (2S,4S)-2-allyl-4-fluoro-pyrrolidine-1,2-dicarboxylate (5.00 g, 17.41 mmol) in acetonitrile (25 mL) and Water (25 mL) was added Trifluoroacetic acid (198.5 mg, 1.74 mmol) and N-Bromosuccinimide (6.20 g, 35.83 mmol) at 0° C., the mixture was stirred at 0° C. for 1 hour. After completion, the resulting solution was adjusted PH to 7 with saturated sodium bicarbonate solution. The resulting solution was extracted with ethyl acetate. The organic layers were washed with brine, dried over anhydrous sodium sulfate and concentrated. The residue was purified by reverse phase chromatography (acetonitrile 0-60/0.1% NH₄HCO₃ in water) to afford 1-(tert-butyl) 2-methyl (2S,4R)-2-(3-bromo-2-hydroxypropyl)-4-fluoropyrrolidine-1,2-dicarboxylate (900 mg, 2.34 mmol, 13% yield) as a red oil. LCMS (ESI) [M+H]⁺=384.1/386.1.

Step 3: methyl (2S,4R)-2-(3-bromo-2-hydroxypropyl)-4-fluoropyrrolidine-2-carboxylate

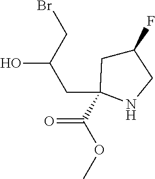

A mixture of 1-(tert-butyl) 2-methyl (2S,4R)-2-(3-bromo-2-hydroxypropyl)-4-fluoropyrrolidine-1,2-dicarboxylate (3.80 g, 9.89 mmol) and 4 M hydrochloric acid in dioxane (8 mL, 32 mmol) in acetonitrile (40 mL) was stirred at room temperature for 1 hour. The solvent was removed under vacuum. The crude was used in the next step without purification. LCMS (ESI, m/z): 284.0 [M+H]⁺.

Step 4: methyl (2R,7aS)-2-fluoro-6-hydroxytetrahydro-1H-pyrrolizine-7a(5H)-carboxylate

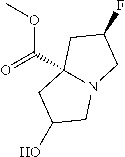

A mixture of methyl (2S,4R)-2-(3-bromo-2-hydroxypropyl)-4-fluoropyrrolidine-2-carboxylate (2.80 g, 9.85 mmol) and potassium carbonate (4.09 g, 29.57 mmol) in acetonitrile (30 mL) was stirred at room temperature for 1 hour. After completion, the solids were filtered out. After filtration, the filtrate was concentrated under reduced pressure. The residue was purified by flash chromatography on silica gel eluting with dichloromethane/methanol (90:10) to afford methyl (2R,7aS)-2-fluoro-6-hydroxytetrahydro-1H-pyrrolizine-7a(5H)-carboxylate (827 mg, 4.07 mmol, 41.3% yield) as a yellow solid. LCMS (ESI, m/z): 204.1 [M+H]⁺.

Step 5: methyl (2R)-2,6-difluorotetrahydro-1H-pyrrolizine-7a(5H)-carboxylate

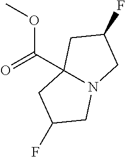

To a mixture of methyl (2R,7aS)-2-fluoro-6-hydroxytetrahydro-1H-pyrrolizine-7a(5H)-carboxylate (700.0 mg, 3.44 mmol) in dichloromethane (10 mL) was added diethylaminosulfur trifluoride (1.66 g, 10.33 mmol), the mixture was stirred for 1 hour at room temperature. The reaction was quenched with methanol. The solvent was removed under vacuum. The residue was purified by flash chromatography on silica gel eluting with dichloromethane/methanol (97:3) to afford methyl (2R)-2,6-difluorotetrahydro-1H-pyrrolizine-7a(5H)-carboxylate (300 mg, 1.46 mmol, 42.4% yield) as yellow oil. LCMS (ESI, m/z): 206.1 [M+H]⁺.

Step 6: ((2R)-2,6-difluorotetrahydro-1H-pyrrolizin-7a(5H)-yl)methanol

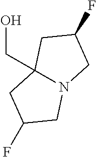 [SMILES: OCC12CC(F)CN1C[C@H](F)C2]

To a mixture of methyl methyl (2R)-2,6-difluorotetrahydro-1H-pyrrolizine-7a(5H)-carboxylate (300.0 mg, 1.46 mmol) in tetrahydrofuran (5 mL) was added lithiumaluminum hydride (150.0 mg, 3.95 mmol), the mixture was stirred for 1 h at 0° C. The reaction was quenched with sodium sulfate decahydrate. The solids were filtered out. After filtration, the filtrate was concentrated under reduced pressure. The crude was used in the next step without purification. LCMS (ESI, m/z): 178.1 [M+H]⁺.

Step 7: (6R)-6-(4-((R)-1-(2-(bis(4-methoxybenzyl)amino)pyridin-3-yl)ethyl)-8-chloro-2-(((2R)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

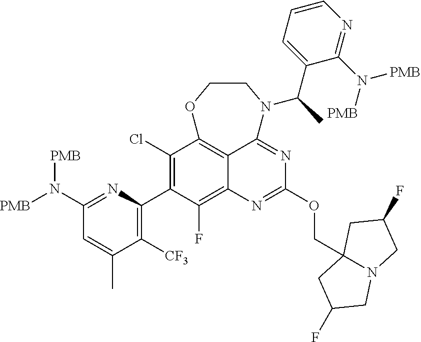

To a mixture of ((2R)-2,6-difluorotetrahydro-1H-pyrrolizin-7a(5H)-yl)methanol (192.6 mg, 1.09 mmol) in tetrahydrofuran (5 mL) was added sodium hydride (67.37 mg, 1.68 mmol, 60% dispersion in mineral oil), the mixture was stirred for 10 min at 0° C. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (380.0 mg, 0.36 mmol) in tetrahydrofuran (2 mL) was added, the mixture was stirred for 4 hours at room temperature. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (96:4) to afford (6R)-6-(4-((R)-1-(2-(bis(4-methoxybenzyl)amino)pyridin-3-yl)ethyl)-8-chloro-2-(((2R)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (270 mg, 0.23 mmol, 62.6% yield) as a yellow solid.

LCMS (ESI, m/z): 1189.45 [M+H]⁺.

Step 8: 6-((R)-4-((R)-1-(2-aminopyridin-3-yl)ethyl)-8-chloro-2-(((2R,6R)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((9R)-4-((R)-1-(2-aminopyridin-3-yl)ethyl)-8-chloro-2-(((2R,6S)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine

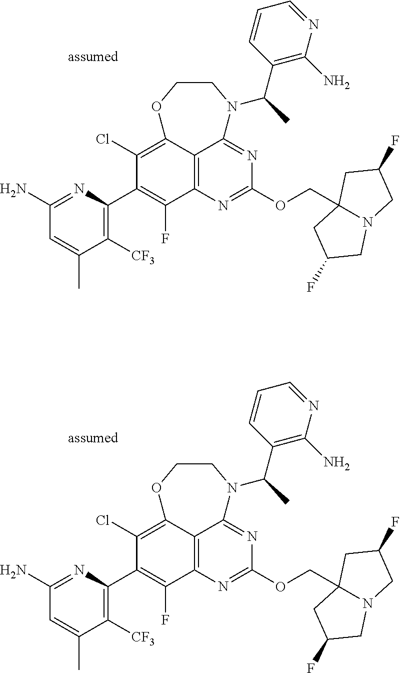

A mixture of (6R)-6-(4-((R)-1-(2-(bis(4-methoxybenzyl)amino)pyridin-3-yl)ethyl)-8-chloro-2-(((2R)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (270.0 mg, 0.23 mmol) trifluoroacetic acid (3 mL) and trifuoromethanesulfonic acid (0.3 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The resulting residue was purified by reverse phase chromatography (acetonitrile/0.1% NH₄HCO₃ in water) to afford crude product. The crude product was purified by Prep-HPLC with the following conditions: Column: Kinetex EVO prep C18, 30*150, 5 μm; Mobile Phase A: Water (10 mmol/L NH₄HCO₃), Mobile Phase B: ACN; Flow rate: 25 mL/min; Gradient: 31% B to 53% B in 10 min, 53% B; Wave Length: 220/254 nm; RT1(min): 12.08 to afford 6-((R)-4-((R)-1-(2-aminopyridin-3-yl)ethyl)-8-chloro-2-(((2R,6R)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine (27.7 mg, 0.04 mmol, 17.2% yield) and 6-((9R)-4-((R)-1-(2-aminopyridin-3-yl)ethyl)-8-chloro-2-(((2R,6S)-2,6-difluorotetrahydro-1H-pyrrolizin-7a(5H)-yl)methoxy)-10-fluoro-5,6-dihydro-4H-[1,4]oxazepino[5,6,7-de]quinazolin-9-yl)-4-methyl-5-(trifluoromethyl)pyridin-2-amine (5.3 mg, 0.007 mmol, 3.3% yield). The stereo chemistry of title compounds was arbitrarily assigned.

Example 98a: ¹H NMR (300 MHz, Methanol-d₄) δ 7.97 (dd, J=5.1, 1.7 Hz, 1H), 7.78 (d, J=7.5 Hz, 1H), 6.80 (dd, J=7.5, 5.1 Hz, 1H), 6.69-6.52 (m, 2H), 5.41 (dt, J=53.3, 3.8 Hz, 2H), 4.48-4.39 (m, 3H), 4.30-4.28 (m, 1H), 3.83-3.61 (m, 1H), 3.58-3.39 (m, 3H), 3.12-2.83 (m, 2H), 2.78-2.52 (m, 2H), 2.46 (s, 3H), 2.20-1.92 (m, 2H), 1.67 (d, J=6.9 Hz, 3H). LCMS (ESI, m/z): 709.2 [M+H]⁺.

Example 98b: ¹H NMR (300 MHz, Methanol-d₄) δ7.97 (dd, J=5.1, 1.7 Hz, 1H), 7.86-7.69 (m, 1H), 6.78 (dd, J=7.5, 5.1 Hz, 1H), 6.61 (s, 1H), 6.54 (q, J=6.8 Hz, 1H), 5.49-5.19 (m, 2H), 4.56-4.39 (m, 3H), 4.36-4.29 (m, 1H), 3.78-3.63 (m, 1H), 3.52-3.47 (m, 2H), 3.38-3.31 (m, 1H), 3.29-3.17 (m, 2H), 2.60-2.12 (m, 7H), 1.67 (d, J=6.9 Hz, 3H). LCMS (ESI, m/z): 709.2 [M+H]⁺.

Example 99a & 99b: 6-((R)-2-(((6S,8S)-4-oxa-1-azabicyclo[4.2.0]octan-8-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine & 6-((R)-2-(((6R,8R)-4-oxa-1-azabicyclo[4.2.0]octan-8-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

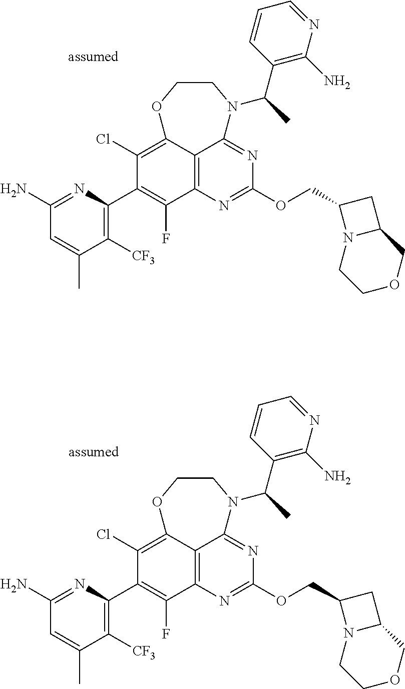

### Synthetic Route

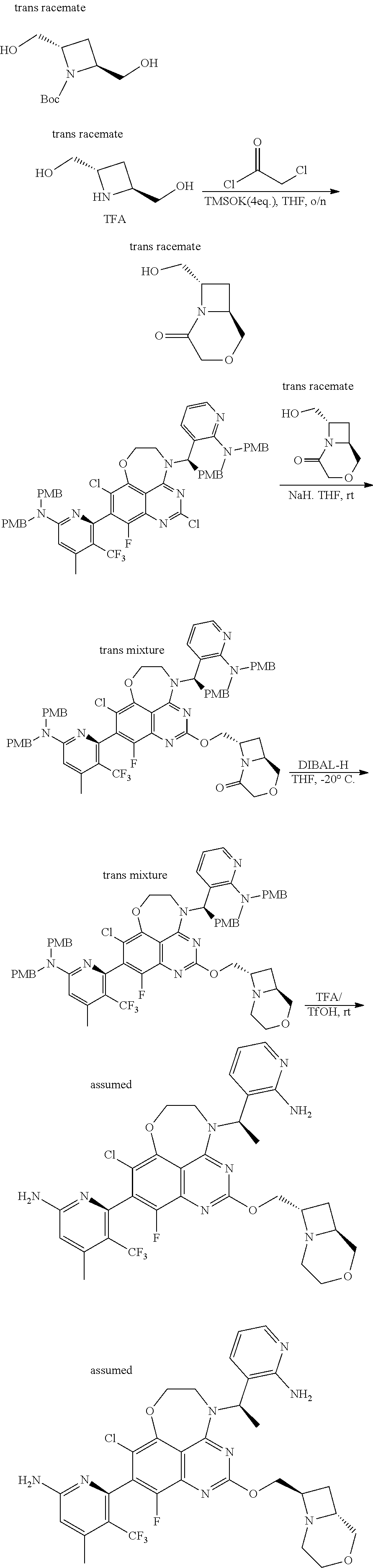

Step 1: ((2S,4S)-azetidine-2,4-diyl)dimethanol 2,2,2-trifluoroacetaldehyde (Trans Mixture)

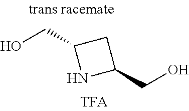

A mixture of tert-butyl (2S,4S)-2,4-bis(hydroxymethyl)azetidine-1-carboxylate (700.0 mg, 3.22 mmol) in dichloromethane (10 mL) and 2,2,2-trifluoroacetic acid (2 mL) was stirred at room temperature for 0.5 hour. After completion, the reaction mixture was concentrated under vacuum. The crude product was used in the next step without purification. LCMS (ESI, m/z): 118.1 [M+H]⁺.

Step 2: (6S,8S)-8-(hydroxymethyl)-4-oxa-1-azabicyclo[4.2.0]octan-2-one (Trans Mixture)

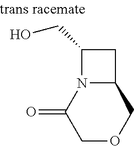 [SMILES: O=C1COC[C@@H]2C[C@@H](CO)N12]

To a mixture of ((2S,4S)-azetidine-2,4-diyl)dimethanol 2,2,2-trifluoroacetaldehyde (trans mixture) (380.0 mg, 3.25 mmol) in tetrahydrofuran (10 mL) was added potassium trimethylsilanolate (1.69 g, 13.17 mmol) and 2-chloroacetyl chloride (1.09 mg, 9.70 mmol), the mixture was stirred for 16 hours at room temperature. After completion, the solvent was removed under vacuum. The residue was purified by flash chromatography on silica gel eluting with dichloromethane/methanol (95:5) to afford (6S,8S)-8-(hydroxymethyl)-4-oxa-1-azabicyclo[4.2.0]octan-2-one (trans mixture) (250 mg, 1.59 mmol, 49% yield) as a yellow solid. LCMS (ESI, m/z): 158.1 [M+H]⁺.

Step 3: (6S,8S)-8-((((R)-9-(6-(bis(4-methoxybenzyl)amino)-4-methyl-3-(trifluoromethyl)pyridin-2-yl)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-2-yl)oxy)methyl)-4-oxa-1-azabicyclo[4.2.0]octan-2-one (Trans Mixture)

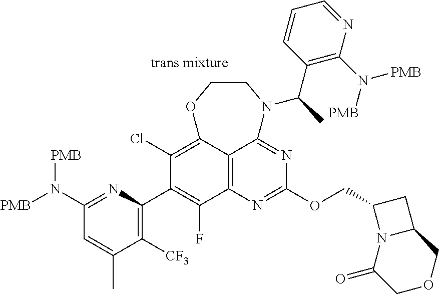

To a mixture of (6S,8S)-8-(hydroxymethyl)-4-oxa-1-azabicyclo[4.2.0]octan-2-one (trans mixture) (195 mg, 1.24 mmol) in tetrahydrofuran (10 mL) was added sodium hydride (130.0 mg, 3.25 mmol, 60% dispersion in mineral oil), the mixture was stirred for 10 min at 0° C. Then 6-[13-[(1R)-1-[2-[bis[(4-methoxyphenyl)methyl]amino]-3-pyridyl]ethyl]-3,8-dichloro-6-fluoro-10-oxa-2,4,13-triazatricyclo[7.4.1.05,14]tetradeca-1,3,5,7,9(14)-pentaen-7-yl]-N,N-bis[(4-methoxyphenyl)methyl]-4-methyl-5-(trifluoromethyl)pyridin-2-amine (650.0 mg, 0.62 mmol) in tetrahydrofuran (1 mL) was added, the mixture was stirred for 1 hour at room temperature. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (96:4) to afford (6S,8S)-8-((((R)-9-(6-(bis(4-methoxybenzyl)amino)-4-methyl-3-(trifluoromethyl)pyridin-2-yl)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-2-yl)oxy)methyl)-4-oxa-1-azabicyclo[4.2.0]octan-2-one (trans mixture) (690 mg, 0.59 mmol, 95.2% yield) as a yellow solid. LCMS (ESI, m/z): 1169.4 [M+H]⁺.

Step 4: 6-((R)-2-(((6S,8S)-4-oxa-1-azabicyclo[4.2.0]octan-8-yl)methoxy)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (trans mixture)

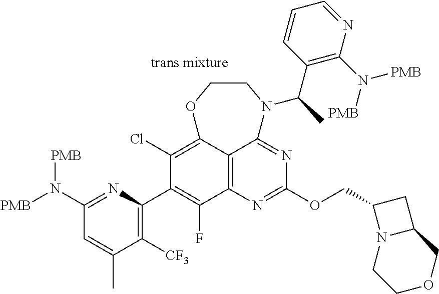

To a mixture of (6S,8S)-8-((((R)-9-(6-(bis(4-methoxybenzyl)amino)-4-methyl-3-(trifluoromethyl)pyridin-2-yl)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-2-yl)oxy)methyl)-4-oxa-1-azabicyclo[4.2.0]octan-2-one (trans mixture) (680.0 mg, 0.58 mmol) in tetrahydrofuran (10 mL) was added diisobutylaluminum hydride (1.74 mL, 1.74 mmol, 1M in toluene), the mixture was stirred for 2 hours at −20° C. The reaction was quenched with sodium sulfate decahydrate. The solids were filtered out. After filtration, the filtrate was concentrated under reduced pressure. The residue was purified by flash chromatography on silica gel eluting with dichloromethane/methanol (96:4) to afford 6-((R)-2-(((6S,8S)-4-oxa-1-azabicyclo[4.2.0]octan-8-yl)methoxy)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (trans mixture) (210 mg, 0.18 mmol, 31.3% yield) as a white solid. LCMS (ESI, m/z): 1155.4 [M+H]⁺.

Step 5: 6-((R)-2-(((6S,8S)-4-oxa-1-azabicyclo[4.2.0]octan-8-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine & 6-((R)-2-(((6R,8R)-4-oxa-1-azabicyclo[4.2.0]octan-8-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

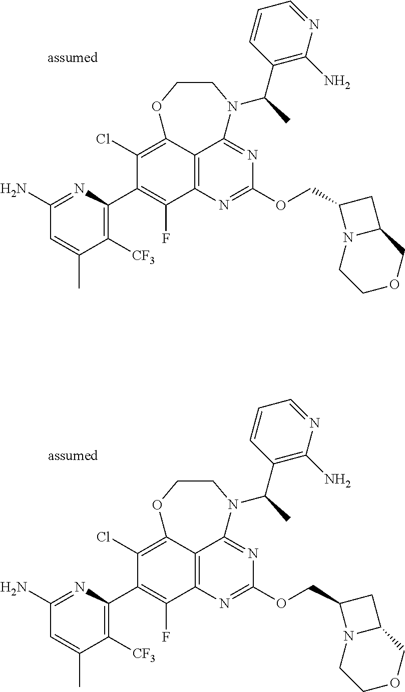

A mixture of 6-((R)-2-(((6S,8S)-4-oxa-1-azabicyclo[4.2.0]octan-8-yl)methoxy)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (trans mixture) (270.0 mg, 0.23 mmol) in trifluoroacetic acid (5 mL) and trifuoromethanesulfonic acid (0.5 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The resulting residue was purified by reverse phase chromatography (acetonitrile/0.1% NH₄HCO₃ in water) to afford crude product. The crude product was purified by Prep-HPLC with the following conditions: Column: XSelect CSH Fluoro Phenyl, 30*150 mm, 5 μm; Mobile Phase A: Water (10 mmol/L NH₄HCO₃), Mobile Phase B: ACN; Flow rate: 60 mL/min; Gradient: 35% B to 60% B in 9 min, 60% B; Wave Length: 254/220 nm; RT1(min): 7.22. The product was purified by Chiral-Prep-HPLC with the following conditions: Column: Lux Sum Cellulose-4, 3*25 cm, 5 μm; Mobile Phase A: CO₂, Mobile Phase B: MEOH (0.1% 2M NH₃-MEOH); Flow rate: 100 mL/min; Gradient: isocratic 55% B; Column Temperature (° C.): 35; Back Pressure (bar): 100: Wave Length: 220 nm; RT1(min): 6.52: RT2(min): 8.07; Sample Solvent: MEOH (0.1% 2M NH₃-MEOH); Injection Volume: 2.5 mL; Number of Runs: 10 to afford 6-((R)-2-(((6S,8S)-4-oxa-1-azabicyclo[4.2.0]octan-8-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (22.4 mg, 0.03 mmol, 14.2% yield) and 6-((R)-2-(((6R,8R)-4-oxa-1-azabicyclo[4.2.0]octan-8-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (21.5 mg, 0.03 mmol, 13.6% yield). The stereo chemistry of title compounds was arbitrarily assigned.

Example 99a: LCMS (ESI, m/z): 675.2 [M+H]⁺. ¹H NMR (300 MHz, Methanol-d₄, ppm) δ 7.98 (dd, J=5.1, 1.7 Hz, 1H), 7.77 (d, J=7.2 Hz, 1H), 6.80 (dd, J=7.5, 5.1 Hz, 1H), 6.61 (s, 1H), 6.59-6.45 (m, 1H), 4.64-4.39 (m, 4H), 4.39-4.22 (m, 1H), 4.05-3.91 (m, 1H), 3.91-3.78 (m, 2H), 3.78-3.59 (m, 3H), 3.59-3.47 (m, 1H), 3.12-2.77 (m, 2H), 2.46 (s, 3H), 2.37-2.21 (m, 1H), 1.93-1.80 (m, 1H), 1.68 (d, J=6.9 Hz, 3H). Chiral HPLC: Column: Lux 3 um Cellulose-4, 4.6*50 mm, 3 um; MeOH (0.1% DEA); Flow rate: 4 mL/min; Retention time: 0.962 (faster peak).

Example 99b: LCMS (ESI, m/z): 675.2 [M+H]⁺. ¹H NMR (300 MHz, Methanol-d₄, ppm) δ 7.98 (dd, J=5.0, 1.7 Hz, 1H), 7.78 (d, J=7.3 Hz, 1H), 6.80 (dd, J=7.5, 5.1 Hz, 1H), 6.61 (s, 1H), 6.53 (q, 1H), 4.64-4.36 (m, 4H), 4.36-4.26 (m, 1H), 4.02-3.79 (m, 3H), 3.79-3.60 (m, 3H), 3.60-3.45 (m, 1H), 3.15-2.76 (m, 2H), 2.46 (s, 3H), 2.39-2.23 (m, 1H), 2.00-1.81 (m, 1H), 1.68 (d, J=6.9 Hz, 3H). Chiral HPLC: Column: Lux 3 um Cellulose-4, 4.6*50 mm, 3 um; MeOH (0.1% DEA); Flow rate: 4 mL/min: Retention time: 1.424 (slower peak).

Example 100a & 100b: 4-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-amine & 4-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-

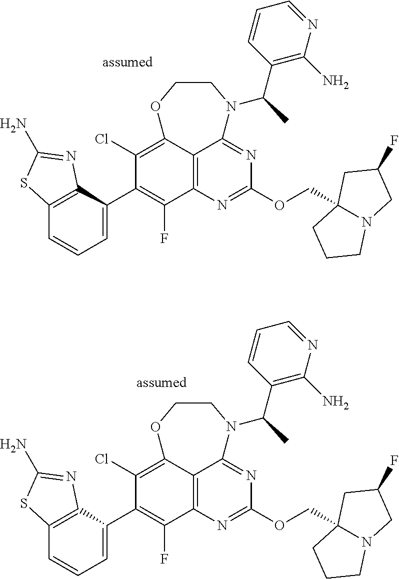

### Synthetic Route

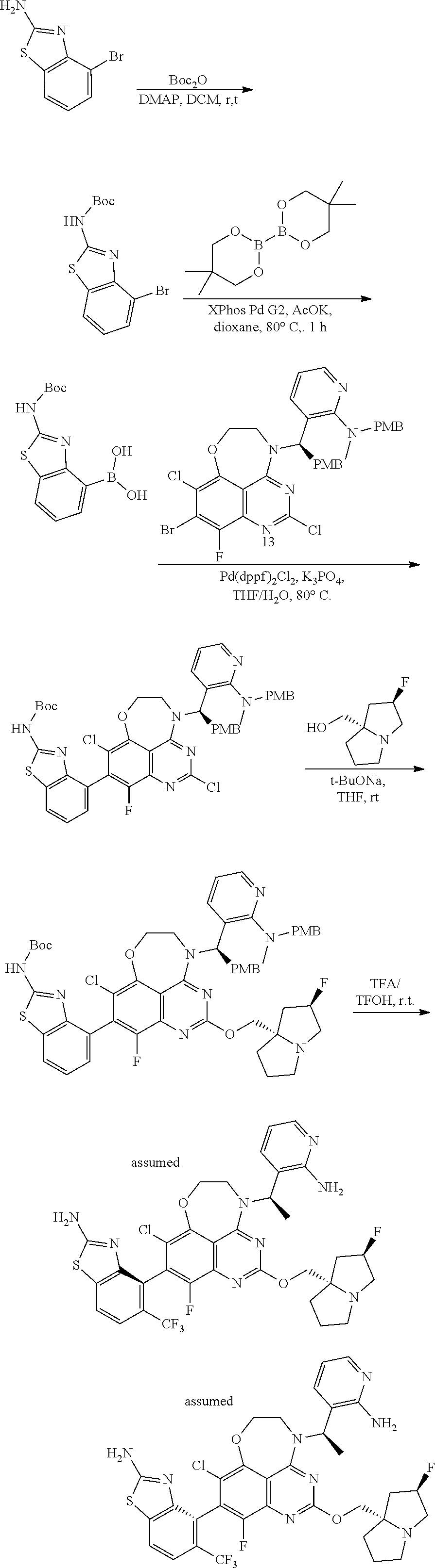

Step 1: tert-butyl (4-bromobenzo[d]Thiazol-2-yl)carbamate

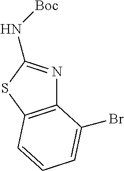 [SMILES: CC(C)(C)OC(=O)Nc1nc2c(Br)cccc2s1]

A mixture of 4-bromo-1,3-benzothiazol-2-amine (4.90 g, 21.39 mmol), di-tert-butyl dicarbonate (9.33 g, 42.78 mmol) and 4-dimethylaminopyridine (5.22 g, 42.78 mmol) in dichloromethane (50 mL) was stirred at 25° C. for 30 minutes. After completion, the resulting solution was diluted with water and extracted with dichloromethane. The organic layers were dried over anhydrous sodium sulfate and concentrated. The residue was purified by flash chromatography on silica gel eluting with petroleum ether/ethyl acetate (3:7) to afford tert-butyl (4-bromobenzo[d]thiazol-2-yl)carbamate (4.13 g, 12.56 mmol, 58.7% yield) as a white solid. LC-MS: (ESI, m/z): 328.9[M+H]⁺

Step 2: (2-((tert-butoxycarbonyl)amino)benzo[d]thiazol-4-yl)boronic acid

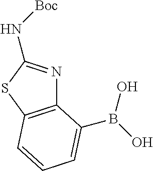 [SMILES: CC(C)(C)OC(=O)Nc1nc2c(B(O)O)cccc2s1]

Under nitrogen, the mixture of tert-butyl (4-bromobenzo[d]thiazol-2-yl)carbamate (1.00 g, 3.04 mmol), bis(neopentylglycolato)diboron (6.86 g, 30.38 mmol), potassium acetate (894.3 mg, 9.11 mmol) and XPhos Pd G2 (477.5 mg, 0.61 mmol) in 1,4-dioxane (10 mL) was stirred at 80° C. for 30 minutes. After completion, the solids were filtered out and washed with dichloromethane. After filtration, the filtrate was concentrated under reduced pressure. The resulting residue was purified by reverse phase chromatography (acetonitrile/0.1% NH₄HCO₃ in water) to afford (2-((tert-butoxycarbonyl)amino)benzo[d]thiazol-4-yl)boronic acid (425 mg, 1.44 mmol, 47.6% yield) as a white solid. LC-MS: (ESI, m/z): 295.1 [M+H]⁺

Step 3: tert-butyl (4-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)benzo[d]thiazol-2-yl)carbamate

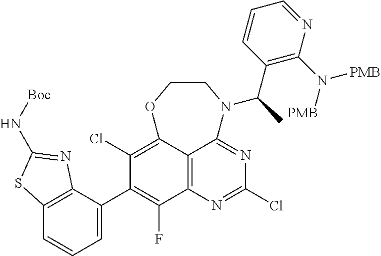

Under nitrogen, a mixture of 3-[(1R)-1-(7-bromo-3,8-dichloro-6-fluoro-10-oxa-2,4,13-triazatricyclo[7.4.1.05,14]tetradeca-1,3,5(14),6,8-pentaen-13-yl)ethyl]-N,N-bis[(4-methoxyphenyl)methyl]pyridin-2-amine (826.0 mg, 1.16 mmol), (2-((tert-butoxycarbonyl)amino)benzo[d]thiazol-4-yl)boronic acid (442.7 mg, 1.51 mmol), 1,1′-bis(diphenylphosphino)ferrocene-Palladium(II)dichloride dichloromethane complex (93.9 mg, 0.12 mmol) and potassium phosphate tribasic (737.4 mg, 3.47 mmol) in tetrahydrofuran (8 mL) and water (1.6 mL) was stirred at 80° C. for 1 hour. After completion, the reaction was diluted with water and extracted with ethyl acetate. The organic layers were washed with brine, dried over anhydrous sodium sulfate and concentrated. The residue was purified by flash chromatography on silica gel eluting with petroleum ether/ethyl acetate (81:19) to afford tert-butyl (4-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)benzo[d]thiazol-2-yl)carbamate (686 mg, 0.78 mmol, 67.24%) as a yellow solid. LC-MS: (ESI, m/z): 882.2 [M+H]⁺

Step 4: tert-butyl (4-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-yl)carbamate

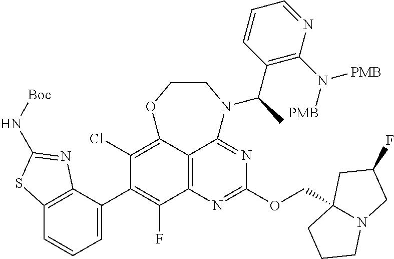

Under nitrogen, to a solution of [(2R,8S)-2-fluoro-1,2,3,5,6,7-hexahydropyrrolizin-8-yl]methanol (486.9 mg, 3.06 mmol) in tetrahydrofuran (10 mL) was added sodium tert-butoxide (293.9 mg, 3.06 mmol), the mixture was stirred at 0° C. for 5 minutes. Then tert-butyl (4-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)benzo[d]thiazol-2-yl)carbamate (900.0 mg, 1.02 mmol) was added and stirred at 25° C. for 30 minutes. After completion, the reaction was quenched with saturated ammonium chloride solution. The resulting solution was diluted with water and extracted with ethyl acetate. The organic layers were washed with brine, dried over anhydrous sodium sulfate and concentrated. The residue was purified by flash chromatography on silica gel eluting with dichloromethane/methanol (91:9) to afford tert-butyl (4-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-yl)carbamate (328 mg, 32.6 mmol, 32.01%) as a yellow solid. LC-MS: (ESI, m/z): 1005.3 [M+H]⁺

Step 5: 4-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-amine & 4-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-amine

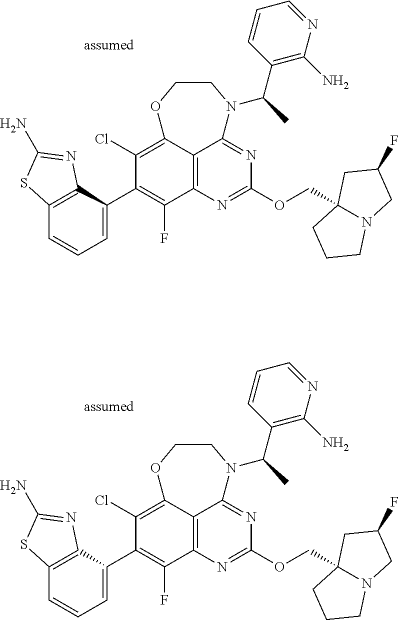

A mixture of tert-butyl (4-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-yl)carbamate (280.0 mg, 0.28 mmol) in 2,2,2-trifluoroacetic acid (2.7 mL) and trifluoromethanesulfonic acid (0.27 mL) was stirred at 25° C. for 15 minutes. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 37% B to 62% B in 9 min, 62% B; Wave Length: 254/220 nm; RT1(min): 8.6 to afford 4-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-amine (17.4 mg, 0.03 mmol, 9.1% yield) and 4-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)benzo[d]thiazol-2-amine (12.9 mg, 0.02 mmol, 6.5% yield).

Example 100a: LC-MS: (ESI, m/z): 665.2 [M+H]⁺, ¹H NMR (300 MHz, Methanol-d₄, ppm) δ 7.96 (dd, J=5.1, 1.7 Hz, 1H), 7.81-7.63 (m, 2H), 7.25-7.11 (m, 2H), 6.78 (dd, J=7.5, 5.1 Hz, 1H), 6.51 (q, J=6.9 Hz, 1H), 5.31 (d, J=52.9 Hz, 1H), 4.51-4.40 (m, 1H), 4.36-4.20 (m, 3H), 3.78-3.64 (m, 1H), 3.58-3.44 (m, 1H), 3.40-3.34 (m, 1H), 3.27-3.16 (m, 2H), 3.09-2.96 (m, 1H), 2.36-2.15 (m, 3H), 2.06-1.86 (m, 3H), 1.68 (d, J=6.9 Hz, 3H).

Example 100b: LC-MS: (ESI, m/z): 665.3 [M+H]⁺, ¹H NMR (300 MHz, Methanol-d₄, ppm) δ 7.97 (d, J=5.1, 1.6 Hz, 1H), 7.81-7.65 (m, 2H), 7.26-7.13 (m, 2H), 6.78 (dd, J=7.5, 5.1 Hz, 1H), 6.53 (q, J=6.8 Hz, 1H), 5.32 (d, J=53.7 Hz, 1H), 4.54-4.42 (m, 1H), 4.40-4.27 (m, 3H), 3.80-3.62 (m, 1H), 3.56-3.43 (m, 1H), 3.37-3.18 (m, 3H), 3.09-2.97 (m, 1H), 2.44-2.33 (m, 1H), 2.32-2.09 (m, 2H), 2.06-1.86 (m, 3H), 1.70 (d, J=6.9 Hz, 3H).

Example 101: 3-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine

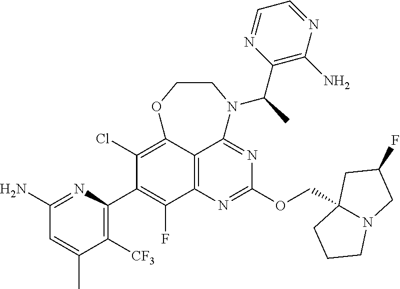

### Synthetic Route

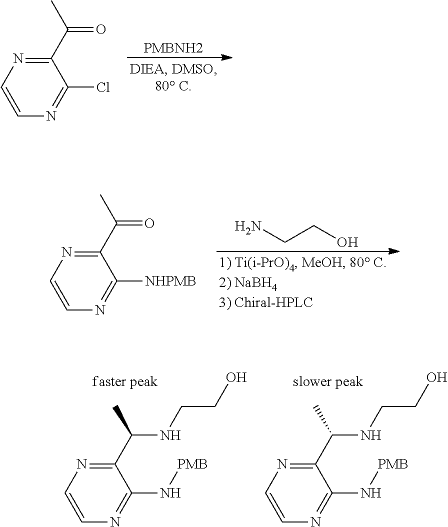

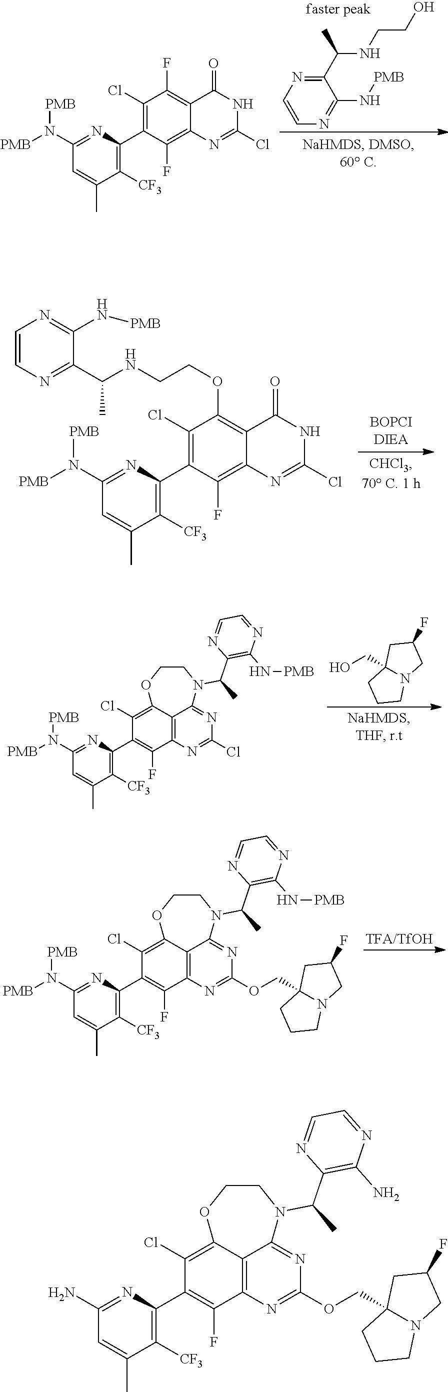

Step 1: 1-(3-((4-methoxybenzyl)amino)pyrazin-2-yl)ethan-1-one

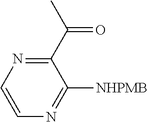

A solution of 1-(3-chloropyrazin-2-yl)ethan-1-one (160.00 g, 1021.90 mmol) and N,N-diisopropylethylamine (533.98 mL, 3065.7 mmol) in dimethyl sulfoxide (1 L). Then 4-methoxybenzylamine (280.37 g, 2043.80 mmol) was added and stirred at 80° C. for 2 hours. After completion, the reaction mixture was diluted with water, extracted with ethyl acetate, washed with water, dried over anhydrous sodium sulfate and concentrated under vacuum. The residue was purified by flash chromatography on silica gel eluting with ethyl acetate/petroleum ether (1:10) to afford the title compound (226.00 g, 852.03 mmol, 83.4% yield). LC-MS: (ESI, m/z): 258.1 [M+H]⁺

Step 2: (R)-2-((1-(3-((4-methoxybenzyl)amino)pyrazin-2-yl)ethyl)amino)ethan-1-ol & (S)-2-((1-(3-((4-methoxybenzyl)amino)pyrazin-2-yl)ethyl)amino)ethan-1-ol

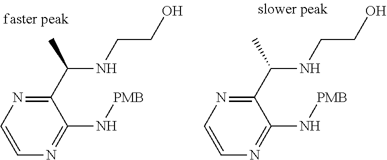

A solution of 1-(3-((4-methoxybenzyl)amino)pyrazin-2-yl)ethan-1-one (80.00 g, 310.93 mmol) and titanium tetraisopropanolate (176.61 g, 621.87 mmol) in methyl alcohol (500 mL) was added 2-aminoethanol (56.3 mL, 932.8 mmol) and stirred 6 hours at 80° C. Then the reaction solution was cooled to the room temperature. Then sodium borohydride (35.26 g, 932.80 mmol) was added and stirred at 25° C. for 12 hours. After completion, the solvent was concentrated under vacuum. The reaction mixture was diluted with dichloromethane and water. After filtration, the filtrate was concentrated under reduced pressure. The residue was purified by flash chromatography on silica gel eluting with methanol/dichloromethane (1:15) to afford the title compound (80.00 g, 261.93 mmol, 84.2% yield) as a yellow oil. The product was separated by Chiral-Prep-HPLC with the following conditions: Column: Lux Sum Cellulose-4, 5*25 cm, 10 μm; Flow rate: 200 mL/min; Gradient: 10% B to 10% B in 20 min; Wave Length: 220 nm to afford (R)-2-((1-(3-((4-methoxybenzyl)amino)pyrazin-2-yl)ethyl)amino)ethan-1-ol (32.2 g, 10.66 mmol, 34.0% yield) (desired isomer) and (S)-2-((1-(3-((4-methoxybenzyl)amino)pyrazin-2-yl)ethyl)amino)ethan-1-ol (35.0 g, 11.59 mmol, 37.2% yield). LC-MS: (ESI, m/z): 303.2 [M+H]⁺

Step 3: 7-((R)-6-(bis(4-methoxybenzyl)amino)-4-methyl-3-(trifluoromethyl)pyridin-2-yl)-2,6-dichloro-8-fluoro-5-(2-(((R)-1-(3-((4-methoxybenzyl)amino)pyrazin-2-yl)ethyl)amino)ethoxy)quinazolin-4(3H)-one

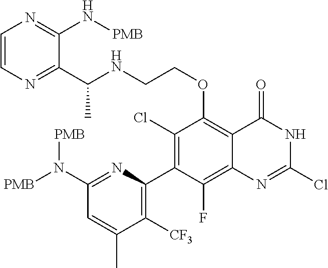

Similar to as described in General Procedure A. Under nitrogen, a solution of (R)-2-((1-(3-((4-methoxybenzyl)amino)pyrazin-2-yl)ethyl)amino)ethan-1-ol (136.3 mg, 0.45 mmol) in dimethyl sulfoxide (2 mL) was added sodium bis(trimethylsilyl)amide (1.2 mL, 1.2 mmol, 1M in tetrahydrofuran) and stirred at 25° C. for 15 min. Then the reaction solution was transferred into a solution of (R)-7-(6-(bis(4-methoxybenzyl)amino)-4-methyl-3-(trifluoromethyl)pyridin-2-yl)-2,6-dichloro-5,8-difluoroquinazolin-4(3H)-one (200.0 mg, 0.3 mmol) in dimethyl sulfoxide (2.0 mL) and stirred at 60° C. for 1 hour. After completion, the reaction solution was quenched with saturated ammonium chloride solution, and diluted with water, extracted with dichloromethane, washed with water, dried over anhydrous sodium sulfate and concentrated under vacuum to afford 7-[6-[bis[(4-methoxyphenyl)methyl]amino]-4-methyl-3-(trifluoromethyl)-2-pyridyl]-2,6-dichloro-8-fluoro-5-[2-[[(1R)-1-[3-[(4-methoxyphenyl)methylamino]pyrazin-2-yl]ethyl]amino]ethoxy]-3H-quinazolin-4-one (600.0 mg, crude) as yellow solid. LC-MS: (ESI, m/z): 947.8 [M+H]⁺

Step 4: 3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine

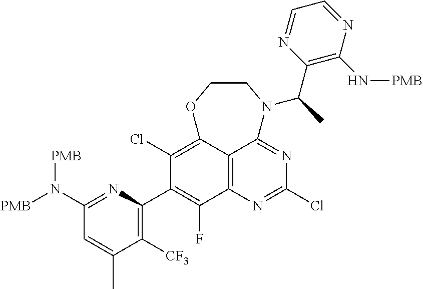

Similar to as described in General Procedure B. A solution of 7-((R)-6-(bis(4-methoxybenzyl)amino)-4-methyl-3-(trifluoromethyl)pyridin-2-yl)-2,6-dichloro-8-fluoro-5-(2-(((R)-1-(3-((4-methoxybenzyl)amino)pyrazin-2-yl)ethyl)amino)ethoxy)quinazolin-4(3H)-one (800.0 mg, crude) and N,N-diisopropylethylamine (447.5 mg, 3.47 mmol) in chloroform (10.0 mL) was added bis(2-oxo-3-oxazolidinyl)phosphinic chloride (330.4 mg, 1.3 mmol) and stirred at 70° C. for 1 hour. After completion, the reaction solution was diluted with dichloromethane, washed with water, dried over anhydrous sodium sulfate and concentrated under vacuum. The residue was purified by flash chromatography on silica gel eluting with dichloromethane/ethyl acetate (4:1) to afford the title compound (388.0 mg, 0.42 mmol, 48.1% yield) as a yellow solid. LC-MS: (ESI, m/z): 929.8 [M+H]⁺

Step 5: 3-((R)-1-((R)-9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine

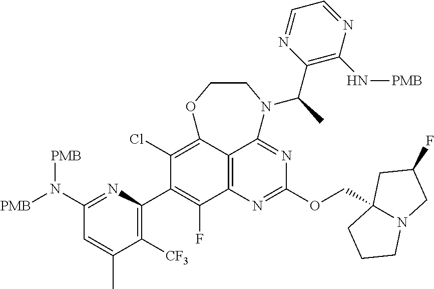

Under nitrogen, a solution of ((2R,7aS)-2-fluorotetrahydro-1H-pyrrolizin-7a(5H)-yl)methanol (197.8 mg, 1.24 mmol) in tetrahydrofuran (6.5 mL) was added sodium bis(trimethylsilyl)amide (1.6 mL, 1.6 mmol, 1M in tetrahydrofuran) and stirred 0.25 hours at 25° C. Then a solution of 3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (330.0 mg, 0.35 mmol) in tetrahydrofuran (6.5 mL) was added at 25° C. and stirred 1 hour at 25° C. After completion, the reaction solution was diluted with dichloromethane, washed with water, dried over anhydrous sodium sulfate and concentrated under vacuum. The residue was purified by flash chromatography on silica gel eluting with dichloromethane/methanol (20:1) to afford the title compound (360.0 mg, 0.32 mmol, 90.6% yield) as a yellow solid. LC-MS: (ESI, m/z): 1052.5 [M+H]⁺

Step 4: 3-((R)-1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine

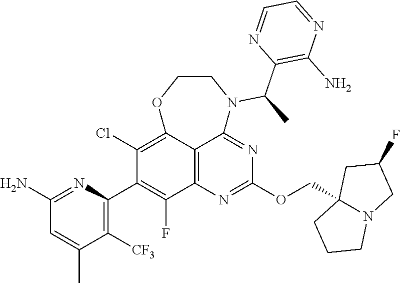

A solution of 3-((R)-1-((R)-9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine (360.0 mg, 0.34 mmol) in trifluoromethanesulfonic acid (0.5 mL) and trifluoroacetic acid (5.0 mL) was stirred at 25° C. for 30 min. After completion, the solution was concentrated under vacuum, diluted with dichloromethane and neutralized with saturated sodium carbonate solution. Then the reaction solution was washed with water, dried over anhydrous sodium sulfate and concentrated under vacuum. The crude product was further purified by Prep-HPLC with the following conditions. Column: Xselect CSH C18 OBD Column 30*150 mm 5 μm, n; Mobile Phase A: Water (10 mmol/L NH4HCO3), Mobile Phase B: ACN; Flow rate: 60 mL/min; Gradient: 33% B to 59% B in 9 min, 59% B; Wave Length: 254/220 nm; RT1(min): 8.18 to afford the title compound (45.1 mg, 0.06 mmol, 18.9% yield). LC-MS: (ESI, m/z): 692.2 [M+H]⁺

Example 101: ¹H NMR (300 MHz, DMSO-d₆, ppm) δ 7.94 (d, J=2.7 Hz, 1H), 7.77 (d, J=2.7 Hz, 1H), 6.81 (s, 2H), 6.47 (s, 1H), 6.40 (s, 2H), 6.27 (q, J=6.7 Hz, 1H), 5.27 (d, J=54.4 Hz, 1H), 4.54 (dd, J=11.5, 6.9 Hz, 1H), 4.35 (dd, J=12.1, 6.1 Hz, 1H), 4.03 (s, 2H), 3.86 (dd, J=15.5, 6.4 Hz, 1H), 3.62 (dd, J=15.6, 6.6 Hz, 1H), 3.20-3.02 (m, 2H), 2.98 (s, 1H), 2.87-2.69 (m, 1H), 2.35 (d, J=2.1 Hz, 3H), 2.16-2.08 (m, 1H), 2.07-1.90 (m, 2H), 1.88-1.67 (m, 3H), 1.57 (d, J=6.8 Hz, 3H).
Example 102a & 102b & 102c & 102d: 5-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine, 5-((R)-1-((R)-9-(6-amino-4-methyl-3-(trifluoromethyl)pyridin-2-yl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine, 5-((R)-1-((S)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine and 5-((R)-1-((S)-9-(6-amino-4-methyl-3-(trifluoromethyl)pyridin-2-yl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine

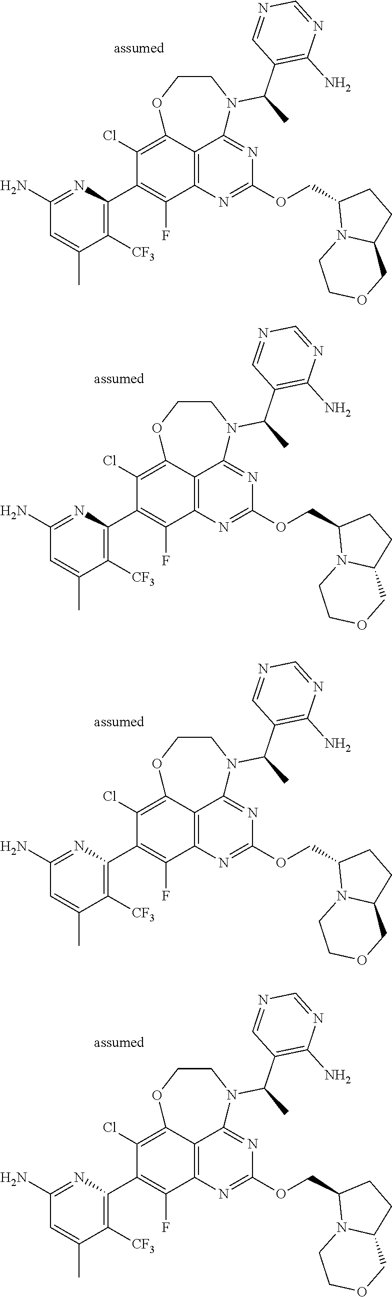

### Synthetic Route:

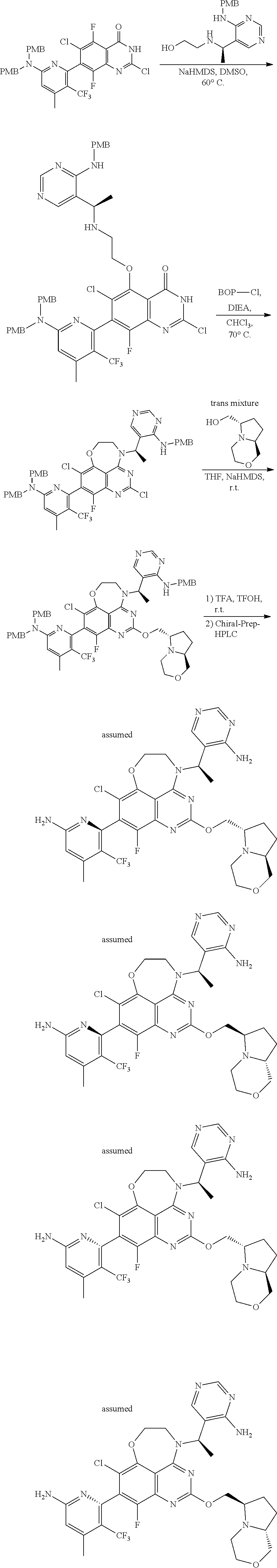

Step 1: 7-(6-(bis(4-methoxybenzyl)amino)-4-methyl-3-(trifluoromethyl)pyridin-2-yl)-2,6-dichloro-8-fluoro-5-(2-(((R)-1-(4-((4-methoxybenzyl)amino)pyrimidin-5-yl)ethyl)amino)ethoxy)quinazolin-4(3H)-one

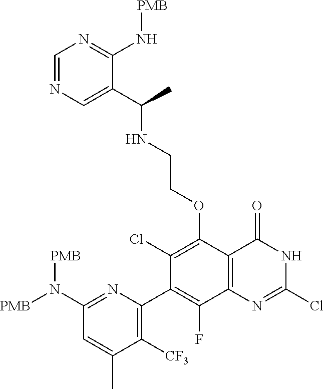

Similar to as described in General Procedure A. Under nitrogen, sodium bis(trimethylsilyl)amide (16 mL, 16 mmol, 1M in tetrahydrofuran) was added into the solution of (R)-2-((1-(4-((4-methoxybenzyl)amino)pyrimidin-5-yl)ethyl)amino)ethan-1-ol (1.50 g, 4.96 mmol) in dimethyl sulfoxide (35 mL) and stirred at room temperature for 0.5 hour. The resulting solution was added into the solution of 7-(6-(bis(4-methoxybenzyl)amino)-4-methyl-3-(trifluoromethyl)pyridin-2-yl)-2,6-dichloro-5,8-difluoroquinazolin-4(3H)-one (3.00 g, 4.51 mmol) in dimethyl sulfoxide (35 mL). Then the resulting solution was stirred for 1 hour at 60° C. After completion, the reaction mixture was diluted with water. The resulting solution was extracted with dichloromethane and washed with brine. The organic layer was dried over anhydrous sodium sulfate and concentrated under vacuum. The crude product was used in the next step without purification. LC-MS: (ESI, m/z): 947.8 [M+H]⁺

Step 2: 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine

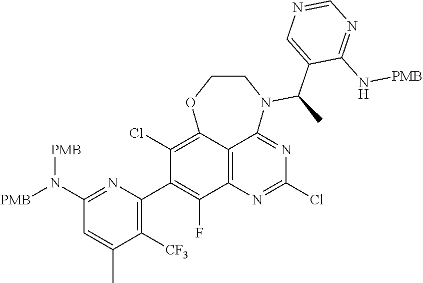

Similar to as described in General Procedure B. A solution of 7-(6-(bis(4-methoxybenzyl)amino)-4-methyl-3-(trifluoromethyl)pyridin-2-yl)-2,6-dichloro-8-fluoro-5-(2-(((R)-1-(4-((4-methoxybenzyl)amino)pyrimidin-5-yl)ethyl)amino)ethoxy)quinazolin-4(3H)-one (37.0 g, 23.42 mmol) and N,N-diisopropylethylamine (16.32 mL, 93.69 mmol) in chloroform (300 mL) was added bis(2-oxo-3-oxazolidinyl)phosphinic chloride (11.9 g, 46.85 mmol) and stirred at 60° C. for 1 hour. After completion, the reaction mixture was diluted with dichloromethane. The organic layer was washed with water and brine. The organic layer was dried over anhydrous sodium sulfate and concentrated under vacuum. The residue was purified by flash chromatography on silica gel eluting with dichloromethane/ethyl acetate (1:2) to afford the title compound (10.00 g, 10.43 mmol, 44.5% yield) as a yellow solid. LC-MS: (ESI, m/z): 929.8 [M+H]⁺

Step 3: 5-((1R)-1-(9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyrimidin-4-amine

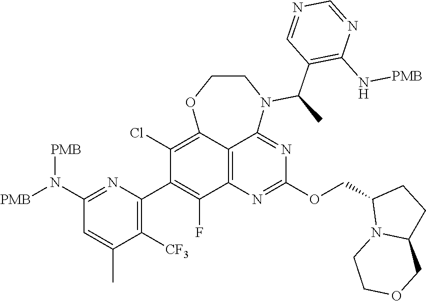

A solution of ((6S,8aS)-hexahydro-1H-pyrrolo[2,1-c][1,4]oxazin-6-yl)methanol (trans mixture) (45.0 mg, 0.29 mmol) in tetrahydrofuran (2 mL) was added sodium bis(trimethylsilyl)amide (0.4 mL, 0.4000 mmol, 1M in tetrahydrofuran) and stirred at 25° C. for 0.5 hour. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (190.0 mg, 0.20 mmol) was added and stirred at 25° C. for 1 hour. After completion, the solvent was concentrated under vacuum. The residue was purified by reverse phase eluting with acetonitrile/water (5-95% in 30 min) to afford the title compound (90.0 mg, 0.086 mmol, 41.9% yield). LC-MS: (ESI, m/z): 1050.5 [M+H]⁺

Step 4: 5-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine, 5-((R)-1-((R)-9-(6-amino-4-methyl-3-(trifluoromethyl)pyridin-2-yl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine, 5-((R)-1-((S)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine and 5-((R)-1-((S)-9-(6-amino-4-methyl-3-(trifluoromethyl)pyridin-2-yl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine

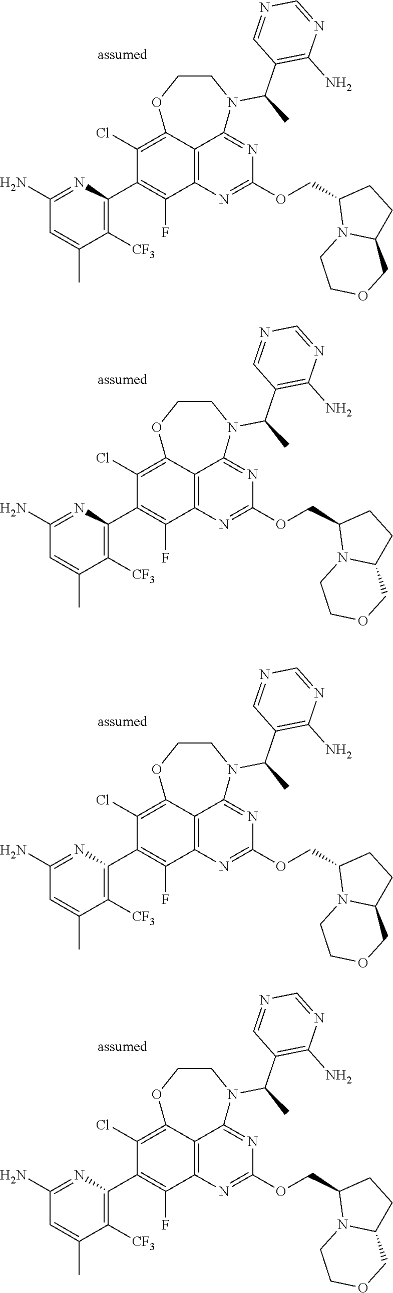

A solution of 5-((1R)-1-(9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyrimidin-4-amine (150.0 mg, 0.1400 mmol) in trifluoroacetic acid (3 mL) and trifluoromethanesulfonic acid (0.3 mL) was stirred at 25° C. for 5 hours. After completion, the solvent was concentrated under vacuum. The residue was purified by Prep-HPLC with the following conditions: Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 25% B to 45% B in 10 min, 45% B; Wave Length: 254/220 nm; RT1(min): 10.66 to afford the two products. The first product was separation by Chiral-Prep-HPLC with the following conditions: Column: CHIRALPAK IG, 2*25 cm, 5 μm; Mobile Phase A: Hex:DCM=3:1(0.5% 2M NH3-MeOH)-HPLC, Mobile Phase B: EtOH-HPLC; Flow rate: 20 mL/min; Gradient: 70% B to 70% B in 21 min; Wave Length: 220/254 nm; RT1(min): 11.369; RT2(min): 17.511; Sample Solvent: ETOH:DCM=1:1; Injection Volume: 1.6 mL; Number Of Runs: 5 to afford 5-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine (13.9 mg, 0.0201 mmol, 14.1% yield) and 5-((R)-1-((R)-9-(6-amino-4-methyl-3-(trifluoromethyl)pyridin-2-yl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine (15.2 mg, 0.0220 mmol, 15.4% yield). The second product was separated by Chiral-Prep-HPLC with the following conditions: Column: CHIRALPAK IE, 2*25 cm, 5 μm; Mobile Phase A: MtBE (0.5% 2M NH3-MeOH)-HPLC, Mobile Phase B: MeOH-HPLC; Flow rate: 20 mL/min; Gradient: 8% B to 8% B in 18 min; Wave Length: 220/254 nm; RT1(min): 10.961; RT2(min): 14.613; Sample Solvent: EtOH-HPLC; Injection Volume: 1 mL; Number Of Runs: 6 to afford 5-((R)-1-((S)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine (20.0 mg, 0.029 mmol, 20.3% yield) and 5-((R)-1-((S)-9-(6-amino-4-methyl-3-(trifluoromethyl)pyridin-2-yl)-8-chloro-10-fluoro-2-(((6R,8aR)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrimidin-4-amine (18.7 mg, 0.027 mmol, 19% yield).

Example 102a: ¹¹H NMR (400 MHz, DMSO-d₆, ppm) δ 8.38 (s, 1H), 8.29 (s, 1H), 6.98-6.60 (m, 4H), 6.48 (s, 1H), 6.14 (q, J=6.9 Hz, 1H), 4.52-4.44 (m, 1H), 4.46-4.34 (m, 2H), 4.14-4.12 (m, 1H), 3.80-3.62 (m, 1H), 3.61-3.34 (m, 5H), 3.13 (t, J=10.4 Hz, 1H), 3.00-2.79 (m, 3H), 2.36 (d, J=2.3 Hz, 3H), 2.13-2.00 (m, 1H), 1.76-1.61 (m, 1H), 1.67-1.48 (m, 4H), 1.35-1.26 (m, 1H). LC-MS: (ESI, m/z): 690.2 [M+H]^{+ Chiral HPLC: Column: XBridge Prep C}18 OBD Column, 30*100 mm, 5 μm; Mobile Phase A: Water (10 mmol/L NH₄HCO₃), Mobile Phase B: ACN; Flow: 60 mL/min; Retention time: 10.66 min (First peak).

Example 102b: ¹H NMR (400 MHz, DMSO-d₆, ppm) δ 8.38 (s, 1H), 8.29 (s, 1H), 6.98-6.60 (m, 4H), 6.48 (s, 1H), 6.14 (q, J=6.9 Hz, 1H), 4.52-4.44 (m, 1H), 4.46-4.34 (m, 2H), 4.14-4.12 (m, 1H), 3.80-3.62 (m, 1H), 3.61-3.34 (m, 5H), 3.13 (t, J=10.4 Hz, 1H), 3.00-2.79 (m, 3H), 2.36 (d, J=2.3 Hz, 3H), 2.13-2.00 (m, 1H), 1.76-1.61 (m, 1H), 1.67-1.48 (m, 4H), 1.35-1.26 (m, 1H). LC-MS: (ESI, m/z): 690.2 [M+H]^{+ Chiral HPLC: Column: XBridge Prep C}18 OBD Column, 30*100 mm, 5 μm; Mobile Phase A: Water (10 mmol/L NH₄HCOs), Mobile Phase B: ACN: Flow: 60 mL/min; Retention time: 10.66 min (second peak).

Example 102c: ¹H NMR (400 MHz, DMSO-da, ppm) δ 8.38 (s, 1H), 8.29 (s, 1H), 6.98-6.60 (m, 4H), 6.48 (s, 1H), 6.14 (q, J=6.9 Hz, 1H), 4.52-4.44 (m, 1H), 4.46-4.34 (m, 2H), 4.14-4.12 (m, 1H), 3.80-3.62 (m, 1H), 3.61-3.34 (m, 5H), 3.13 (t, J=10.4 Hz, 1H), 3.00-2.79 (m, 3H), 2.36 (d, J=2.3 Hz, 3H), 2.13-2.00 (m, 1H), 1.76-1.61 (m, 1H), 1.67-1.48 (m, 4H), 1.35-1.26 (m, 1H). LC-MS: (ESI, m/z): 690.2 [M+H]^{+ Chiral HPLC: Column: CHIRALPAK IE-}3, 4.6*50 mm, 3 μm; Mobile Phase A: MtBE (0.1% DEA):MeOH=92:8; Flow rate: 1 mL/min (first peak).

Example 102d: ¹H NMR (400 MHz, DMSO-d₆, ppm) δ 8.38 (s, 1H), 8.29 (s, 1H), 6.98-6.60 (m, 4H), 6.48 (s, 1H), 6.14 (q, J=6.9 Hz, 1H), 4.52-4.44 (m, 1H), 4.46-4.34 (m, 2H), 4.14-4.12 (m, 1H), 3.80-3.62 (m, 1H), 3.61-3.34 (m, 5H), 3.13 (t, J=10.4 Hz, 1H), 3.00-2.79 (m, 3H), 2.36 (d, J=2.3 Hz, 3H), 2.13-2.00 (m, 1H), 1.76-1.61 (m, 1H), 1.67-1.48 (m, 4H), 1.35-1.26 (m, 1H). LC-MS: (ESI, m/z): 690.2 [M+H]^{+ Chiral HPLC: Column: CHIRALPAK IE-}3, 4.6*50 mm, 3 μm; Mobile Phase A: MtBE (0.1% DEA):MeOH=92:8; Flow rate: 1 mL/min (second peak).

Example 103a & 103b: 5-((R)-1-((R)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrimidin-4-amine and 5-((R)-1-((S)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrimidin-4-amine

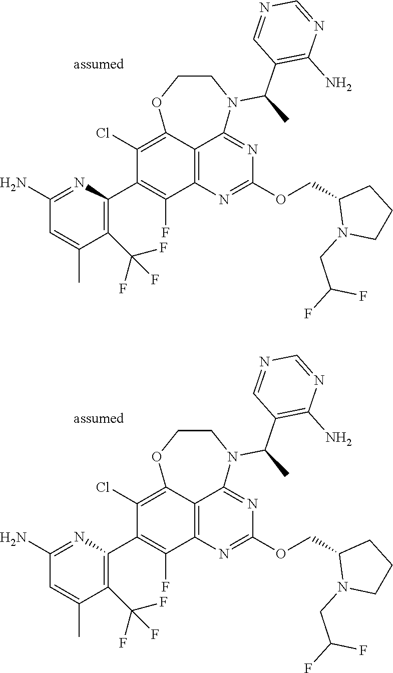

### Synthetic Route:

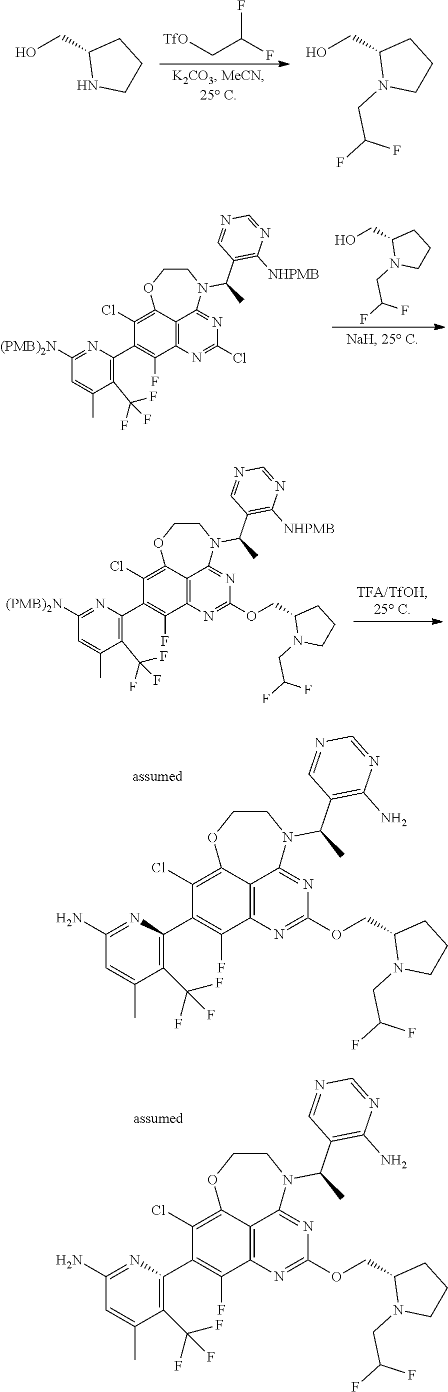

Step 1: (S)-(1-(2,2-difluoroethyl)pyrrolidin-2-yl)methanol

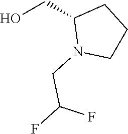 [SMILES: OC[C@@H]1CCCN1CC(F)F]

A solution of I-prolinol (0.2 mL, 1.98 mmol) and potassium carbonate (820.0 mg, 5.93 mmol) in acetonitrile (8 mL) was stirred at 25° C. for 10 minutes. Then 2,2-difluoroethyl trifluoromethanesulfonate (508.0 mg, 2.37 mmol) was added and stirred at 25° C. for 8 hours. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (9:1) to afford (S)-(1-(2,2-difluoroethyl)pyrrolidin-2-yl)methanol (847.0 mg, 5.13 mmol, 25.9% yield) as a yellow oil. LC-MS: (ESI, m/z): 166.2 [M+H]⁺

Step 2: 5-((1R)-1-(9-(6-(bis(4-methoxybenzyl)amino)-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine

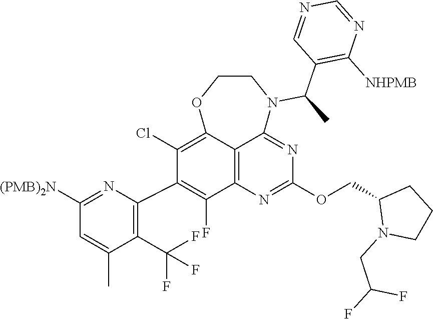

A solution of (S)-(1-(2,2-difluoroethyl)pyrrolidin-2-yl)methanol (320.0 mg, 1.94 mmol) in tetrahydrofuran (6 mL) was added sodium hydride (129.0 mg, 3.23 mmol, 60% dispersion in mineral oil) was stirred at 0° C. for 10 minutes. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (600.0 mg, 0.65 mmol) was added and stirred at 25° C. for 1 hour. After completion, the resulting solution was quenched with saturated ammonium chloride solution, diluted with ethyl acetate and washed with water. The organic layer was washed with brine and dried over anhydrous sodium sulfate and concentrated under vacuum to afford the title compound (669.0 mg, crude) as a brown oil. LC-MS: (ESI, m/z): 1058.6 [M+H]⁺

Step 3: 5-((R)-1-((R)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrimidin-4-amine and 5-((R)-1-((S)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrimidin-4-amine

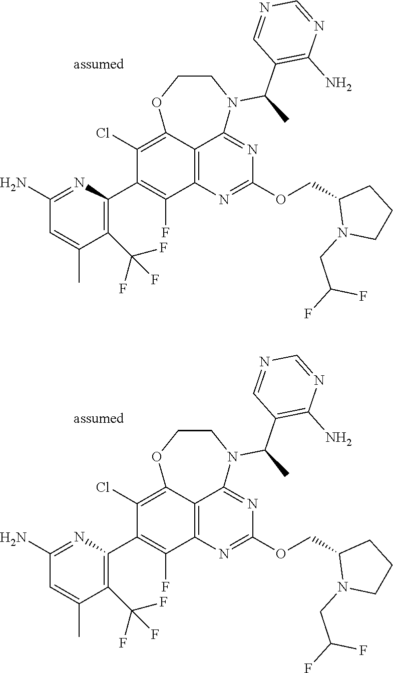

A solution of 5-((1R)-1-(9-(6-(bis(4-methoxybenzyl)amino)-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine (600.0 mg, 0.57 mmol) in trifluoroacetic acid (5 mL) and trifluoromethanesulfonic acid (0.5 mL) was stirred at 25° C. for 6 hours. After completion, the mixture was concentrated under vacuum. The residue was diluted with dichloromethane, adjusted to pH=7 by saturated sodium bicarbonate solution, washed with brine. The organic phase was dried over anhydrous sodium sulfate and concentrated under vacuum. The crude product was purified by Prep-HPLC with the following conditions: Column: Xselect CSH C18 OBD Column 30*150 mm 5 μm, n; Mobile Phase A: Water (0.1% FA), Mobile Phase B: ACN; Flow rate: 60 mL/min; Gradient: 6% B to 36% B in 8 min, 36% B; Wave Length: 254/220 nm; RT1(min): 8 to afford 5-((R)-1-((S)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrimidin-4-amine (77.6 mg, 0.11 mmol, 19.4% yield) and 5-((R)-1-((R)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrimidin-4-amine (79.5 mg, 0.11 mmol, 19.6% yield).

Example 103a: ¹H NMR (300 MHz, DMSO-d₆, ppm) δ 8.38 (s, 1H), 8.30 (s, 1H), 6.81 (s, 2H), 6.75 (s, 2H), 6.48 (s, 1H), 6.31-5.84 (m, 2H), 4.46 (dd, J=11.3, 6.6 Hz, 1H), 4.40-4.23 (m, 2H), 4.12 (dd, J=10.8, 6.9 Hz, 1H), 3.70 (dd, J=15.4, 6.7 Hz, 1H), 3.56-3.38 (m, 1H), 3.37-3.19 (m, 1H), 3.17-2.95 (m, 2H), 2.93-2.69 (m, 1H), 2.47-2.37 (m, 1H), 2.36 (s, 3H), 2.01-1.84 (m, 1H), 1.82-1.62 (m, 3H), 1.60 (d, J=6.8 Hz, 3H). LC-MS: (ESI, m/z): 698.3 [M+H]⁺

Example 103b: ¹H NMR (300 MHz, DMSO-d₆, ppm) δ 8.37 (s, 1H), 8.29 (s, 1H), 6.81 (s, 4H), 6.48 (s, 1H), 6.34-5.82 (m, 2H), 4.55 (dd, J=12.2, 6.5 Hz, 1H), 4.43-4.20 (m, 2H), 4.13 (dd, J=10.8, 7.0 Hz, 1H), 3.77 (dd, J=15.2, 6.2 Hz, 1H), 3.45 (dd, J=16.0, 7.0 Hz, 1H), 3.29-3.16 (m, 1H), 3.16-2.95 (m, 2H), 2.92-2.63 (m, 1H), 2.47-2.39 (m, 1H), 2.36 (s, 3H), 2.02-1.82 (m, 1H), 1.82-1.68 (m, 2H), 1.61 (d, J=6.8 Hz, 4H). LC-MS: (ESI, m/z): 698.2 [M+H]⁺

Example 104a & 104b: 3-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrazin-2-amine

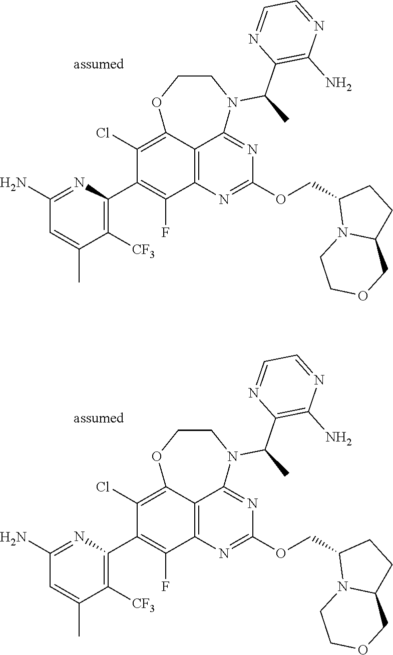

### Synthetic Route

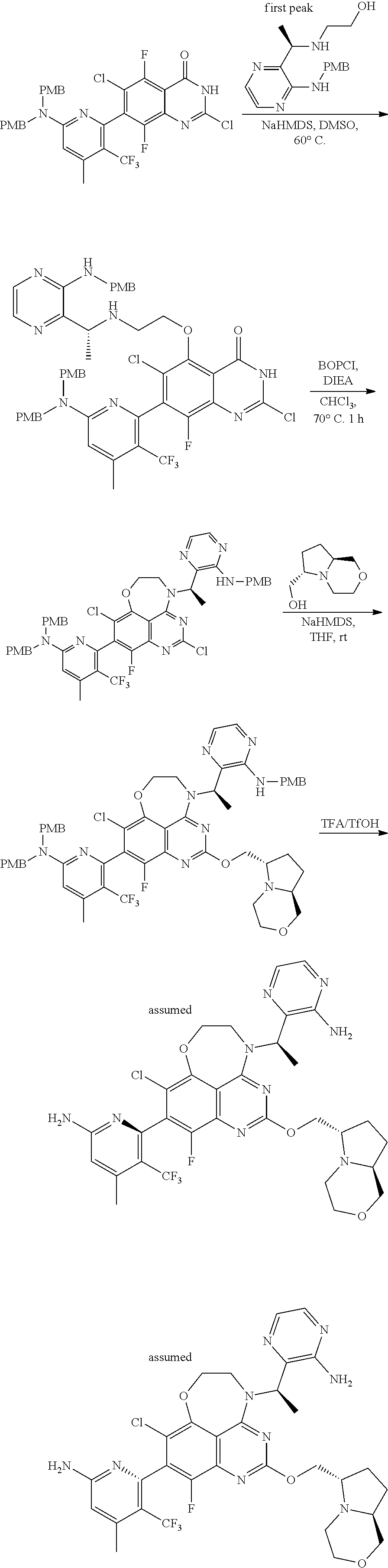

Step 1: 7-(6-(bis(4-methoxybenzyl)amino)-4-methyl-3-(trifluoromethyl)pyridin-2-yl)-2,6-dichloro-8-fluoro-5-(2-(((R)-1-(3-((4-methoxybenzyl)amino)pyrazin-2-yl)ethyl)amino)ethoxy)quinazolin-4(3H)-one

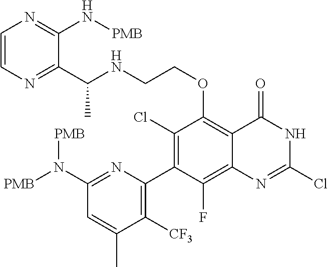

Similar to as described in General Procedure A. Under nitrogen, a solution of (R)-2-((1-(3-((4-methoxybenzyl)amino)pyrazin-2-yl)ethyl)amino)ethan-1-ol (654.3 mg, 2.16 mmol) in dimethyl sulfoxide (12.0 mL) was added sodium bis(trimethylsilyl)amide (7.2 mL, 7.2 mmol, 1M in tetrahydrofuran) and stirred for time at 25° C. for 15 minutes. Then a solution of 7-[6-[bis[(4-methoxyphenyl)methyl]amino]-4-methyl-3-(trifluoromethyl)-2-pyridyl]-2,6-dichloro-5,8-difluoro-3H-quinazolin-4-one (1.20 g, 1.80 mmol) in dimethyl sulfoxide (12.0 mL) was added and stirred at 60° C. for 1 hour. After completion, the reaction solution was quenched with saturated ammonium chloride solution, diluted with water, extracted with dichloromethane, washed with water, dried over anhydrous sodium sulfate and concentrated under vacuum to afford the title compound (1.6 g, crude) as a yellow oil. LC-MS: (ESI, m/z): 947.3 [M+H]⁺

Step 2: 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine

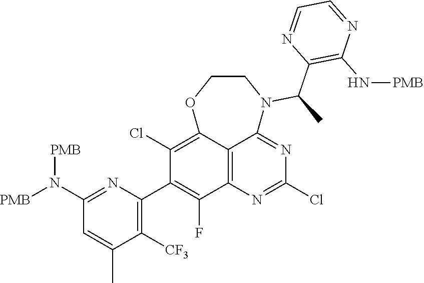

Similar to as described in General Procedure B. A solution of 7-(6-(bis(4-methoxybenzyl)amino)-4-methyl-3-(trifluoromethyl)pyridin-2-yl)-2,6-dichloro-8-fluoro-5-(2-(((R)-1-(3-((4-methoxybenzyl)amino)pyrazin-2-yl)ethyl)amino)ethoxy)quinazolin-4(3H)-one (1.60 g, crude) and N,N-diisopropylethylamine (872.7 mg, 6.77 mmol) in chloroform (20.0 mL) was added bis(2-oxo-3-oxazolidinyl)phosphinic chloride (644.4 mg, 2.54 mmol) and stirred at 70° C. for 1 hour. After completion, the reaction solution was diluted with dichloromethane, washed with water, dried over anhydrous sodium sulfate and concentrated under vacuum. The residue was purified by flash chromatography on silica gel eluting with dichloromethane/ethyl acetate (9:1) to afford the title compound (248.0 mg, 0.27 mmol, 15.8% yield). LC-MS: (ESI, m/z): 929.3 [M+H]⁺

Step 3: 3-((1R)-1-(9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyrazin-2-amine

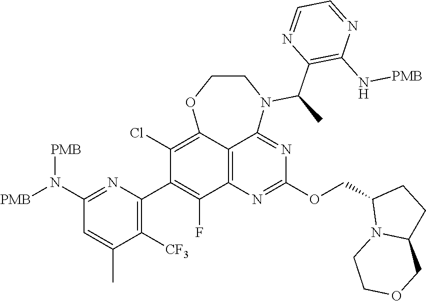

A solution of ((6S,8aS)-hexahydro-1H-pyrrolo[2,1-c][1,4]oxazin-6-yl)methanol (83.9 mg, 0.53 mmol) was added sodium hydride (42.7 mg, 1.07 mmol, 60% purity) in tetrahydrofuran (2.0 mL) and stirred at 25° C. for 10 minutes. Then a solution of N-[(4-methoxyphenyl)methyl]-3-[rac-(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5(14),6,8-pentaen-13-yl]ethyl]pyrazin-2-amine (248.0 mg, 0.27 mmol) in tetrahydrofuran (2.0 mL) was added and stirred at 25° C. for 3 hours. After completion, the reaction was quenched with saturated ammonium chloride solution. The reaction was diluted with ethyl acetate, washed with saturated brine, dried over anhydrous sodium sulfate and concentrated under vacuum to afford the title compound (404.0 mg, crude) as a yellow oil. LC-MS: (ESI, m/z): 1050.4 [M+H]⁺

Step 4: 3-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrazin-2-amine

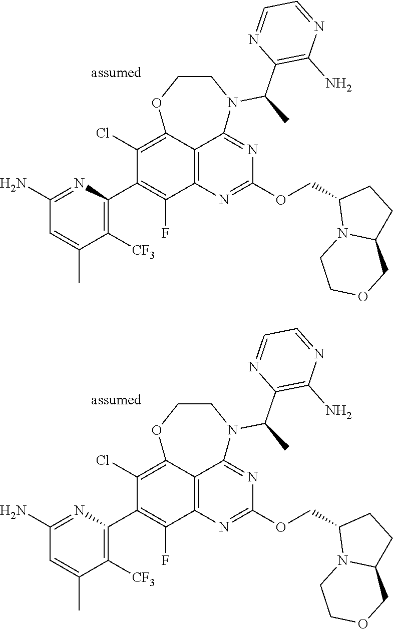

A solution of 3-((1R)-1-(9-(6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyrazin-2-amine (126.0 mg, 0.12 mmol) in trifluoroacetic acid (5.0 mL) and trifluoromethanesulfonic acid (0.5 mL) was stirred at 25° C. for 0.5 hour. After completion, the solution was concentrated under vacuum, diluted with ethyl acetate and neutralized by sodium carbonate solution. Then the reaction solution was washed with water, dried over anhydrous sodium sulfate and concentrated under vacuum. The residue was purified by Prep-HPLC with the following conditions. Column: Xselect CSH C18 OBD Column 30*150 mm 5 μm, n; Mobile Phase A: Water (10 mmol/L NH4HCO3), Mobile Phase B: ACN; Flow rate: 60 mL/min; Gradient: 33% B to 59% B in 9 min, 59% B; Wave Length: 254/220 nm; RT1(min): 8.18 to afford 3-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrazin-2-amine (8.3 mg, 0.01 mmol, 9.7% yield) and 3-((R)-1-((S)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyrazin-2-amine (7.3 mg, 0.01 mmol, 8.9% yield) as a light yellow oil.

Example 104a: ¹H NMR (300 MHz, DMSO-d₆, ppm) δ 7.94 (d, J=2.7 Hz, 1H), 7.77 (d, J=2.7 Hz, 1H), 6.81 (s, 2H), 6.47 (s, 1H), 6.36 (s, 2H), 6.26 (q, J=6.7 Hz, 1H), 4.54 (dd, J=12.0, 6.6 Hz, 1H), 4.46-4.24 (m, 2H), 4.14 (dd, J=10.9, 5.5 Hz, 1H), 3.86 (dd, J=15.7, 6.5 Hz, 1H), 3.71-3.34 (m, 5H), 3.12 (t, J=10.3 Hz, 1H), 3.01-2.75 (m, 3H), 2.36 (d, J=2.2 Hz, 3H), 2.17-1.95 (m, 1H), 1.85-1.67 (m, 1H), 1.65-1.46 (m, 4H), 1.42-1.18 (m, 1H). LC-MS: (ESI, m/z): 690.2 [M+H]⁺

Example 104b: ¹H NMR (300 MHz, DMSO-d₆, ppm) δ 7.93 (d, J=2.7 Hz, 1H), 7.76 (d, J=2.7 Hz, 1H), 6.81 (s, 2H), 6.46 (d, J=10.7 Hz, 3H), 6.23 (q, J=7.0 Hz, 1H), 4.71-4.54 (m, 1H), 4.50-4.11 (m, 3H), 4.10-3.85 (m, 2H), 3.83-3.41 (m, 5H), 3.21-2.75 (m, 3H), 2.36 (d, J=2.2 Hz, 3H), 2.29-2.01 (m, 1H), 1.99-1.72 (m, 2H), 1.59 (d, J=6.8 Hz, 4H). LC-MS: (ESI, m/z): 690.2 [M+H]⁺

Example 106a & 105b: 3-((R)-1-((S)-8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-((R)-8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

### Synthetic Route

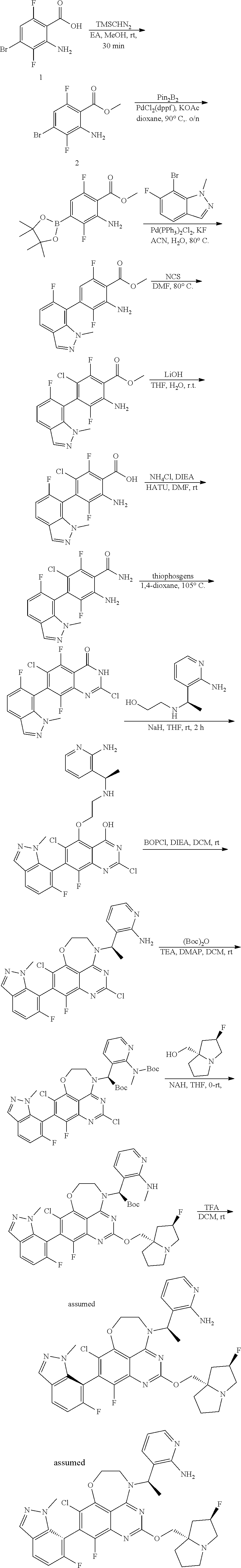

Step 1: methyl 2-amino-4-bromo-3,6-difluorobenzoate

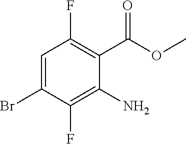

A solution of 2-amino-4-bromo-3,6-difluoro-benzoic acid (2.72 g, 10.78 mmol,) in ethyl acetate (13.5 mL) and methyl alcohol (13.5 mL) was added (trimethylsilyl)diazomethane (10.8 mL, 21.6 mmol, 2 mol/L in n-hexane) at 0° C. and stirred at 25° C. for 10 mins. The solvent was concentrated under vacuum to afford the title compound (2.8 g, crude) as a yellow solid. The crude was used for next step without further purification. LC-MS: (ESI, m/z): 266.0 [M+H]⁺

Step 2: methyl 2-amino-3,6-difluoro-4-(4,4,5,5-tetramethyl-1,3,2-dioxaborolan-2-yl)benzoate

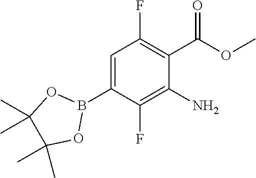

Under nitrogen, a solution of methyl 2-amino-4-bromo-3,6-difluoro-benzoate (2.8 g, crude), bis(pinacolato)diboron (4.11 g, 16.2 mmol), [1,1′-bis(diphenylphosphino)ferrocene]dichloropalladium(II) (787.8 mg, 1.08 mmol) and potassium acetate (3.17 g, 32.34 mmol) in 1,4-dioxane (72 mL) was stirred for 1 h at 100° C. After completion, the reaction was cooled to the room temperature and filtrated. After filtration, the filtrate was concentrated under reduced pressure. The residue was purified by flash chromatography on silica gel, eluted with petroleum ether/ethyl acetate (5:1) to afford crude product. The crude was dissolved in 10 mL petroleum ether and stirred for 10 mins. After filtration, the solid was collect (repeat three times) to yield 2.01 g (59% yield) of the title compound as a white solid. LC-MS: (ESI, m/z): 314.1 [M+H]⁺.

Step 3: methyl 2-amino-3,6-difluoro-4-(6-fluoro-1-methyl-1H-indazol-7-yl)benzoate

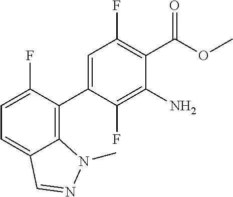 [SMILES: COC(=O)c1c(F)cc(-c2c(F)ccc3cnn(C)c23)c(F)c1N]

Under nitrogen, a solution of methyl 2-amino-3,6-difluoro-4-(4,4,5,5-tetramethyl-1,3,2-dioxaborolan-2-yl)benzoate (1.64 g, 5.24 mmol), 7-bromo-6-fluoro-1-methyl-indazole (1.44 g, 6.3 mmol), bis(triphenylphosphine)palladium(11) chloride (368.2 mg, 0.520 mmol) and potassium fluoride (913 mg, 15.7 mmol) in acetonitrile (20 mL) and water (4 mL) was stirred for 1 h at 80° C. After filtration, the filtrate was concentrated under reduced pressure. The residue was purified by flash chromatography on silica gel, eluted with petroleum ether/ethyl acetate (2:1) to afford 1.25 g (71% yield) of the title compound as a yellow solid. LC-MS: (ESI, m/z): 336.1 [M+H]⁺.

Step 4: methyl 2-amino-5-chloro-3,6-difluoro-4-(6-fluoro-1-methyl-1H-indazol-7-yl)benzoate

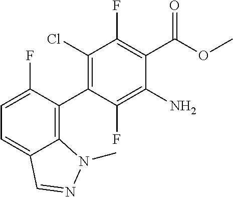 [SMILES: COC(=O)c1c(N)c(F)c(-c2c(F)ccc3cnn(C)c23)c(Cl)c1F]

Under nitrogen, a solution of methyl 2-amino-3,6-difluoro-4-(6-fluoro-1-methyl-indazol-7-yl)benzoate (1.21 g, 3.60 mmol) and N-chlorosuccinimide (574 mg, 4.32 mmol) in N,N-dimethylformamide (12 mL) was stirred at 80° C. for 1 h. The reaction was quenched with sodium thiosulfate (aq) and diluted with ethyl acetate (100 mL). The resulting solution was washed with water, dried over anhydrous sodium sulfate and concentrated under vacuum. The residue was purified by flash chromatography on silica gel, eluted with petroleum ether/ethyl acetate (2:1) to afford 1.1 g (83% yield) of the title compound as a yellow solid. LC-MS: (ESI, m/z): 370.0 [M+H]⁺.

Step 5: 2-amino-5-chloro-3,6-difluoro-4-(6-fluoro-1-methyl-1H-indazol-7-yl)benzoic acid

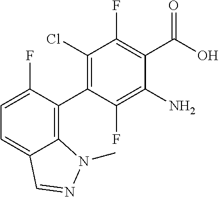 [SMILES: Cn1ncc2ccc(F)c(-c3c(F)c(N)c(C(=O)O)c(F)c3Cl)c21]

A solution of methyl 2-amino-5-chloro-3,6-difluoro-4-(6-fluoro-1-methyl-indazol-7-yl)benzoate (1.10 g, 3.08 mmol) in tetrahydrofuran (12 mL) was added lithium hydroxide (212 mg, 9.23 mmol) in water (4 mL) and stirred over night at 25° C. The reaction mixture was adjust pH to 5-6 with hydrochloric acid (1M). The resulting solution was extracted with ethyl acetate. The combined organic layers were dried over anhydrous sodium sulfate and concentrated under vacuum to afford 1.00 g (crude) of title compound. The crude was used for next step without further purification. LC-MS: (ESI, m/z): 356.0 [M+H]⁺

Step 6: 2-amino-5-chloro-3,6-difluoro-4-(6-fluoro-1-methyl-1H-indazol-7-yl)benzamide

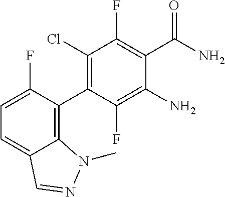 [SMILES: Cn1ncc2ccc(F)c(-c3c(F)c(N)c(C(N)=O)c(F)c3Cl)c21]

A solution of 2-amino-5-chloro-3,6-difluoro-4-(6-fluoro-1-methyl-1H-indazol-7-yl)benzoic acid (1.00 g, crude), ammonium chloride (830.0 mg, 15.4 mmol), 2-(7-Azabenzotriazol-1-yl)-N,N,N′,N′-tetramethyluronium hexafluorophosphate (1.75 g, 4.61 mmol) and N,N-diisopropylethylamine (2.78 g, 21.53 mmol) in N,N-dimethylformamide (15 mL) was stirred for 3 h at 25° C. The resulting solution was diluted with ethyl acetate and washed with ammonium chloride aq. The organic layers were dried over anhydrous sodium sulfate and concentrated under vacuum. The residue was purified by flash chromatography on silica gel, eluted with dichloromethane/methanol (20:1) to afford 950 mg (87% yield) of the title compound as a white solid. LC-MS: (ESI, m/z): [M+H]⁺=355.0.

Step 7: 2,6-dichloro-5,8-difluoro-7-(6-fluoro-1-methyl-1H-indazol-7-yl)quinazolin-4(3H)-one

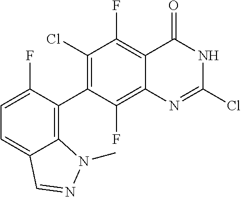 [SMILES: Cn1ncc2ccc(F)c(-c3c(Cl)c(F)c4c(=O)[nH]c(Cl)nc4c3F)c21]

A solution of 2-amino-5-chloro-3,6-difluoro-4-(6-fluoro-1-methyl-1H-indazol-7-yl)benzamide (1.01 g, 2.85 mmol) in 1,4-dioxane (20 mL) was dropwise added thiophosgene (0.65 mL, 8.55 mmol) and stirred at 25° C. for 1 h and 105° C. for 1 h. The solvent was concentrated under vacuum. The residue was purified by flash chromatography on silica gel, eluted with dichloromethane/methanol (20:1) to afford the title compound (1.27 g, crude, 76% purity) as a brown solid. LC-MS: (ESI, m/z): 399.0 [M+H]⁺

Step 8: 5-(2-(((R)-1-(2-aminopyridin-3-yl)ethyl)amino)ethoxy)-2,6-dichloro-8-fluoro-7-(6-fluoro-1-methyl-1H-indazol-7-yl)quinazolin-4-ol

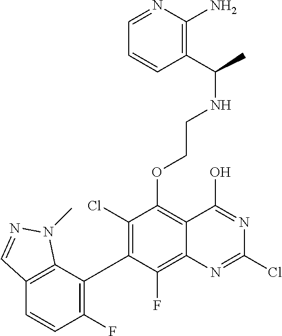 [SMILES: C[C@@H](NCCOc1c(Cl)c(-c2c(F)ccc3cnn(C)c23)c(F)c2nc(Cl)nc(O)c12)c1cccnc1N]

Similar to as described in General Procedure A. To a solution of (R)-2-((1-(2-aminopyridin-3-yl)ethyl)amino)ethan-1-ol (91.00 mg, 0.50 mmol, 1.00 equiv) in tetrahydrofuran (3 mL) was added sodium hydride (120.0 mg, 3.0 mmol, 60% purity) at 0° C. The mixture was stirred for 30 min at room temperature. Then the reaction solution was transferred into 2,6-dichloro-5,8-difluoro-7-(6-fluoro-1-methylindazol-7-yl)quinazolin-4-ol (200.41 mg, 0.502 mmol, 1.00 equiv) in tetrahydrofuran (3 mL) and stirred for 2 hours at rt. The reaction mixture was quenched by water and extracted with dichloromethane. The combined organic layers were dried over anhydrous sodium sulfate, and concentrated under vacuum. The residue was purified by flash chromatography on silica gel eluting with dichloromethane/methanol (20:1) to afford the title compound (73.0 mg, 25.94% yield) as a light yellow solid. LC-MS: (ESI, m/z): 560.1 [M+H]⁺

Step 9: 3-((1R)-1-(2,8-dichloro-10-fluoro-9-(6-fluoro-1-methyl-1H-indazol-7-yl)-5,6-dihydro-4H-[1,4]oxazepino[5,6,7-de]quinazolin-4-yl)ethyl)pyridin-2-amine

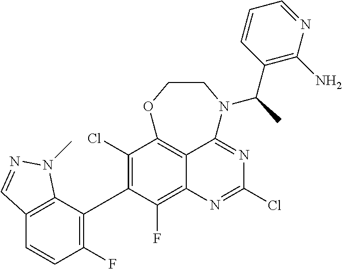 [SMILES: C[C@H](c1cccnc1N)N1CCOc2c(Cl)c(-c3c(F)ccc4cnn(C)c34)c(F)c3nc(Cl)nc1c23]

Similar to as described in General Procedure B. A solution of 5-(2-(((R)-1-(2-aminopyridin-3-yl)ethyl)amino)ethoxy)-2,6-dichloro-8-fluoro-7-(6-fluoro-1-methyl-1H-indazol-7-yl)quinazolin-4-ol (90.0 mg, 0.16 mmol), bis(2-oxo-3-oxazolidinyl)phosphinic chloride (43.1 mg, 0.17 mmol) and N,N-diisopropylethylamine (415.14 mg, 3.22 mmol) in dichloromethane (3 mL) was stirred for 6 h at room temperature under nitrogen atmosphere. After completion, the resulting mixture was concentrated under reduced pressure. The residue was purified by flash chromatography on silica gel eluting with dichloromethane/methanol (20:1) to afford the title compound (80.0 mg, 0.15 mmol, 92% yield) as a yellow solid. LC-MS: (ESI, m/z): 542.1 [M+H]⁺

Step 10: tert-butyl N-tert-butoxycarbonyl-N-[3-[(1R)-1-[3,8-dichloro-6-fluoro-7-(6-fluoro-1-methyl-indazol-7-yl)-10-oxa-2,4,13-tiazatricyclo[7.4.1.05,14]tetradeca-1,3,5,7,9(14)-pentaen-13-yl]ethyl]-2-pyridyl]carbamate

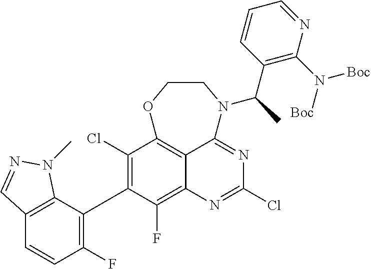 [SMILES: C[C@H](c1cccnc1N(C(=O)OC(C)(C)C)C(=O)OC(C)(C)C)N1CCOc2c(Cl)c(-c3c(F)ccc4cnn(C)c34)c(F)c3nc(Cl)nc1c23]

A solution of 3-((1R)-1-(2,8-dichloro-10-fluoro-9-(6-fluoro-1-methyl-1H-indazol-7-yl)-5,6-dihydro-4H-[1,4]oxazepino[5,6,7-de]quinazolin-4-yl)ethyl)pyridin-2-amine (90.0 mg, 0.17 mmol), triethylamine (167.9 mg, 1.66 mmol), 4-dimethylaminopyridine (12.2 mg, 0.10 mmol) and di-tert-butyl dicarbonate (543.2 mg, 2.49 mmol) in dichloromethane (5 mL) was stirred for 1 day at room temperature. After completion, the resulting mixture was concentrated under reduced pressure. The resulting mixture was diluted with dichloromethane, washed with water, dried over anhydrous sodium sulfate and concentrated under vacuum. The residue was purified by flash chromatography on silica gel eluting with dichloromethane/methanol (10:1) to afford the title compound (70.0 mg, 0.094 mmol, 56.8% yield). LC-MS: (ESI, m/z): 742.6 [M+H]⁺

Step 11: tert-butyl (3-((1R)-1-(8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate

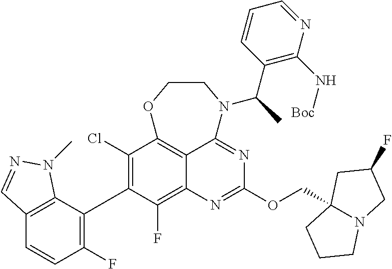 [SMILES: C[C@H](c1cccnc1NC(=O)OC(C)(C)C)N1CCOc2c(Cl)c(-c3c(F)ccc4cnn(C)c34)c(F)c3nc(OC[C@@]45CCCN4C[C@H](F)C5)nc1c23]

To a solution of ((2R,7aS)-2-fluorotetrahydro-1H-pyrrolizin-7a(5H)-yl)methanol (19.3 mg, 0.121 mmol) in tetrahydrofuran (3 mL) was added sodium hydride (12.8 mg, 0.32 mmol 60% purity) at 0° C. and stirred for 30 min. Then the reaction solution was transferred into the solution of tert-butyl N-tert-butoxycarbonyl-N-[3-[(1R)-1-[3,8-dichloro-6-fluoro-7-(6-fluoro-1-methyl-indazol-7-yl)-10-oxa-2,4,13-triazatricyclo[7.4.1.05,14]tetradeca-1,3,5,7,9(14)-pentaen-13-yl]ethyl]-2-pyridyl]carbamate (60.0 mg, 0.081 mmol,) in tetrahydrofuran (2 mL) and stirred at 25° C. for 1 h. After completion, the reaction mixture was quenched by saturated ammonium chloride solution and extracted with dichloromethane. The combined organic extracts were dried over anhydrous sodium sulfate, and concentrated under vacuum. The residue was purified by flash chromatography on silica gel eluting with dichloromethane/methanol (10:1) to afford the title compound (36.0 mg, 58.2% yield) as a yellow solid. LC-MS: (ESI, m/z): 765.0 [M+H]⁺.

Step 12: 3-((R)-1-((S)-8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine & 3-((R)-1-((R)-8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine

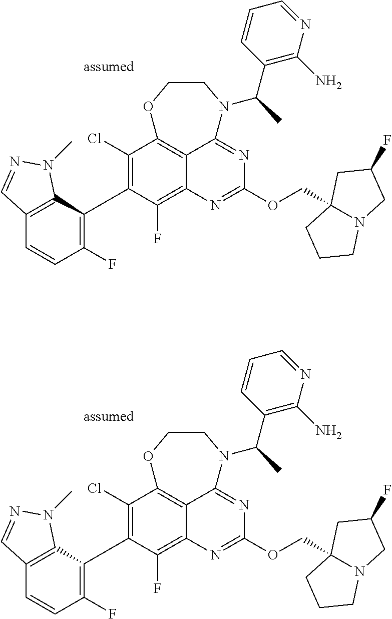

A solution of tert-butyl (3-((1R)-1-(8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (36.0 mg, 0.047 mmol, 1.00 equiv) and trifluoroacetic acid (0.25 mL) in dichloromethane (0.75 mL) was stirred for 10 min. After completion, the resulting mixture was concentrated under vacuum. Then residue was diluted with dichloromethane, washed with sodium carbonate solution, dried over anhydrous sodium sulfate, concentrated under vacuum and purified by flash chromatography on silica gel eluting with dichloromethane/methanol (10:1) to afford the product. The product was purified by Prep-HPLC with the following conditions (Column: XBridge Prep OBD C18 Column, 30*150 mm, 5 μm; Mobile phase, A: Water (10 mmol/L NH4HCO3), B: ACN (43%˜73% in 7 min)). The faster peak is 2.4 mg (0.0036 mmol, 7.7% yield) of 3-((R)-1-((S)-8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine and the slower peak is 2.5 mg (0.0037 mmol, 7.8% yield) of 3-((R)-1-((R)-8-chloro-10-fluoro-9-(6-fluoro-1-methyl-1H-indazol-7-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-amine. LC-MS: (ESI, m/z): 665.3 [M+H]⁺.

Example 105a: ¹H NMR (300 MHz, DMSO-d₆, ppm) δ 8.18 (s, 1H), 8.03-7.91 (m, 2H), 7.64 (d, J=7.2 Hz, 1H), 7.22 (t, J=9.3 Hz, 1H), 6.67 (dd, J=7.5, 4.8 Hz, 1H), 6.30 (d, J=6.9 Hz, 1H), 5.72 (s, 2H), 5.28 (d, J=54.3 Hz, 1H), 4.53 (dd, J=12.1, 6.0 Hz, 1H), 4.33 (dd, J=12.1, 6.7 Hz, 1H), 4.10 (d, J=2.0 Hz, 2H), 3.73 (dd, J=15.9, 6.8 Hz, 1H), 3.56 (s, 3H), 3.43 (dd, J=15.8, 6.4 Hz, 1H), 3.20-3.02 (m, 2H), 2.99 (s, 1H), 2.91-2.76 (m, 1H), 2.21-2.09 (m, 1H), 2.09-1.91 (m, 2H), 1.91-1.67 (m, 3H), 1.59 (d, J=6.8 Hz, 3H).

Example 105b: ¹H NMR (300 MHz, DMSO-d₆, ppm) δ 8.18 (s, 1H), 8.03-7.91 (m, 2H), 7.64 (d, J=7.2 Hz, 1H), 7.22 (t, J=9.3 Hz, 1H), 6.67 (dd, J=7.5, 4.8 Hz, 1H), 6.30 (d, J=6.9 Hz, 1H), 5.72 (s, 2H), 5.28 (d, J=54.3 Hz, 1H), 4.53 (dd, J=12.1, 6.0 Hz, 1H), 4.33 (dd, J=12.1, 6.7 Hz, 1H), 4.10 (d, J=2.0 Hz, 2H), 3.73 (dd, J=15.9, 6.8 Hz, 1H), 3.56 (s, 3H), 3.43 (dd, J=15.8, 6.4 Hz, 1H), 3.20-3.02 (m, 2H), 2.99 (s, 1H), 2.91-2.76 (m, 1H), 2.21-2.09 (m, 1H), 2.09-1.91 (m, 2H), 1.91-1.67 (m, 3H), 1.59 (d, J=6.8 Hz, 3H).

Example 106: 6-((R)-4-((R)-1-(2-aminopyridin-3-yl)ethyl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

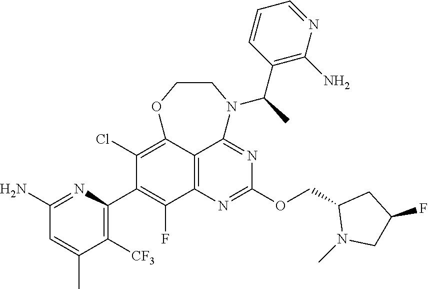

### Synthetic Route

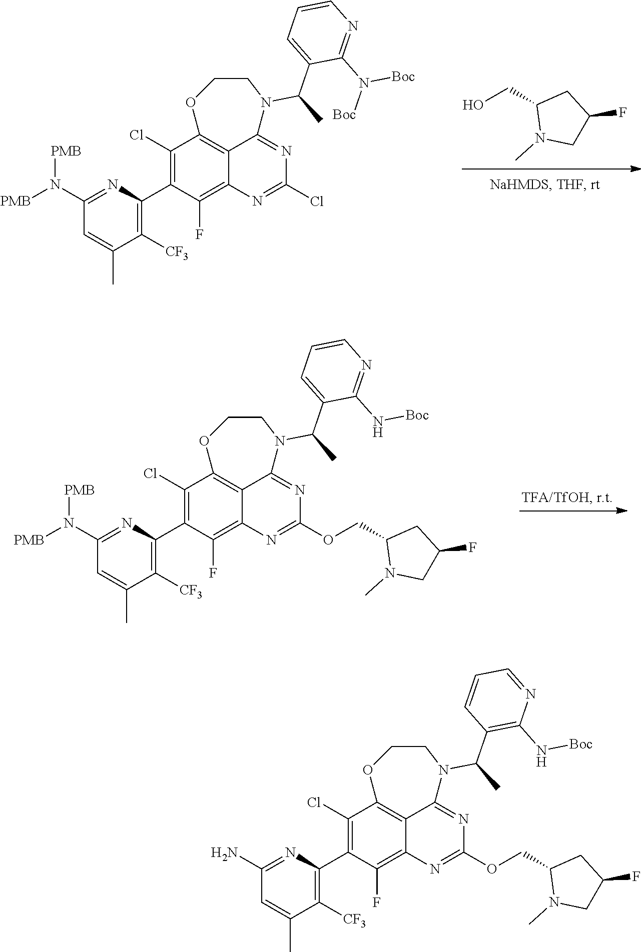

Step 1: tert-butyl (3-((R)-1-(9-((R)-6-(bis(4-methoxybenzyl)amino)-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyridin-2-yl)carbamate

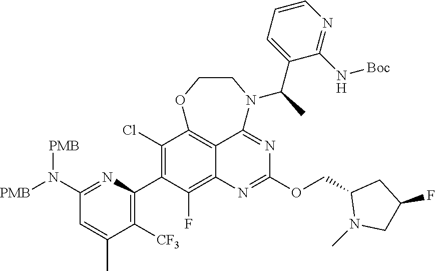

Under nitrogen, a solution of [(2 S,4R)-4-fluoro-1-methyl-pyrrolidin-2-yl]methanol (528.0 mg, 3.97 mmol) in tetrahydrofuran (8 mL) was added sodium bis(trimethylsilyl)amide (4.0 mL, 4.00 mmol, 1M in tetrahydrofuran) and stirred for 10 minutes at 25° C. Then tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]-N-tert-butoxycarbonyl-carbamate (desired atropisomer) (800.0 mg, 0.79 mmol) was added and stirred at 25° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with methanol/dichloromethane (8:1) to afford the title compound (560.0 mg, 0.45 mmol, 56.9% yield) as a yellow solid. LCMS (ESI, m/z): 1005.4 [M+H]⁺.

Step 2: 6-((R)-4-((R)-1-(2-aminopyridin-3-yl)ethyl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

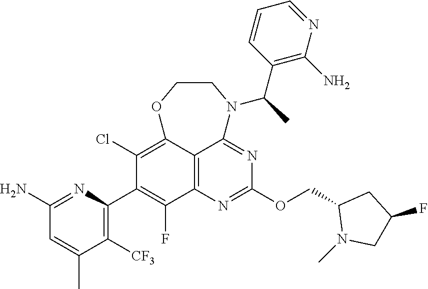

A solution of tert-butyl (3-((R)-1-(9-((R)-6-(bis(4-methoxybenzyl)amino)-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyridin-2-yl)carbamate (540.0 mg, 0.54 mmol) in trifluoroacetic acid (5 mL) and trifuoromethanesulfonic acid (0.5 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: Column: XBridge Prep OBD C18 Column, 30*150 mm, 5 μm; Mobile Phase A: Water (10 mmol/L NH₄HCOs), Mobile Phase B: ACN; Flow rate: 60 mL/min; Gradient: 33% B to 48% B in 10 min, 48% B; Wave Length: 254/220 nm; RT1(min): 9.25 to afford the title compound (112.2 mg, 0.16 mmol, 30.5% yield). LCMS (ESI, m/z): 665.3 [M+H]⁺.

Example 106: ¹H NMR (300 MHz, DMSO-d₆, ppm) δ 7.95 (d, J=4.6 Hz, 1H), 7.62 (d, J=7.5 Hz, 1H), 6.80 (s, 2H), 6.72-6.59 (m, 1H), 6.46 (s, 1H), 6.23 (q, J=6.8 Hz, 1H), 5.68 (s, 2H), 5.18 (d, J=56.1 Hz, 1H), 4.54-4.35 (m, 2H), 4.35-4.13 (m, 2H), 3.63 (dd, J=15.7, 6.9 Hz, 1H), 3.50-3.35 (m, 2H), 3.04-2.83 (m, 1H), 2.60-2.50 (m, 1H), 2.39 (s, 3H), 2.35 (s, 3H), 2.24-1.77 (m, 2H), 1.55 (d, J=6.8 Hz, 3H).

Example 107: (S)-6-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

### Synthetic Route

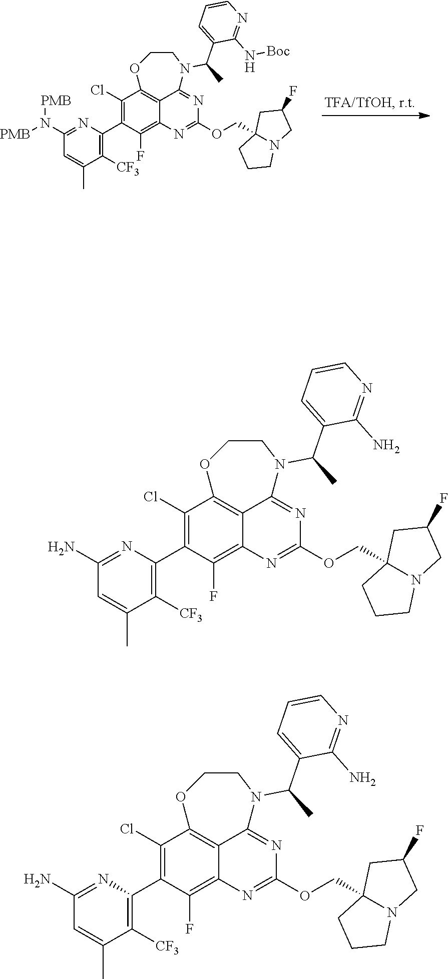

Step 1: (R)-6-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & (S)-6-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

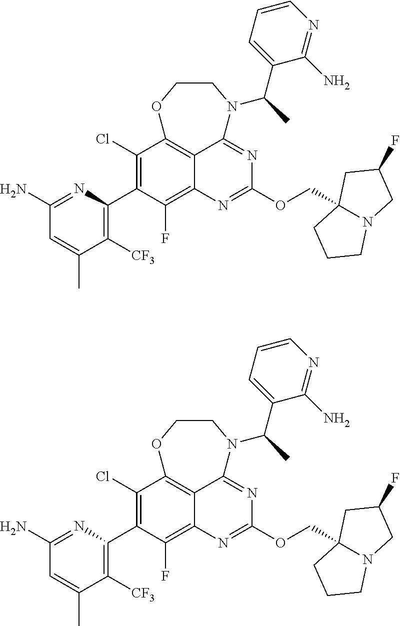

A solution of tert-butyl (3-((1R)-1-(9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (67.0 g, 65.04 mmol) in trifluoroacetic acid (60 mL) and trifluoromethanesulfonic acid (6 mL) was stirred at room temperature for 10 min. After completion, the solvent was concentrated under vacuum, diluted with ethyl acetate, washed with saturated sodium carbonate solution and dried over anhydrous sodium sulfate and concentrated under vacuum. The residue was purified by flash chromatography on silica gel eluting with dichloromethane/methanol (10:1). And the product was further purified by reverse phase with the following condition: water (0.1% ammonium bicarbonate) (A)/acetonitrile (B) Gradient: 5% B to 42% B in 30 min to afford (R)-6-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (20.4 g, 29.6 mmol, 45.5% yield) and (S)-6-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (3.0 g, 4.34 mmol, 6.67% yield). LCMS (ESI, m/z): 691.4 [M+H]⁺.

Example 107: ¹H NMR (400 MHz, Methanol-d₄, ppm) 7.95 (dd, J=5.1, 1.7 Hz, 1H), 7.75 (d, J=7.6 Hz, 1H), 6.77 (dd, J=7.5, 5.1 Hz, 1H), 6.63-6.57 (m, 1H), 6.53 (q, J=6.8 Hz, 1H), 5.37 (d, J=53.6 Hz, 1H), 4.48 (dd, J=12.3, 6.5 Hz, 1H), 4.39-4.24 (m, 3H), 3.73 (dd, J=15.8, 7.2 Hz, 1H), 3.55-3.44 (m, 1H), 3.44-3.33 (m, 2H), 3.28-3.20 (m, 1H), 3.15-3.01 (m, 1H), 2.44 (s, 3H), 2.41-2.13 (m, 3H), 2.12-1.87 (m, 3H), 1.66 (d, J=6.9 Hz, 3H)

Example 108a & 108b: 6-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-5-(trifluoromethyl)pyridin-2-amine & 6-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-5-(trifluoromethyl)pyridin-2-amine

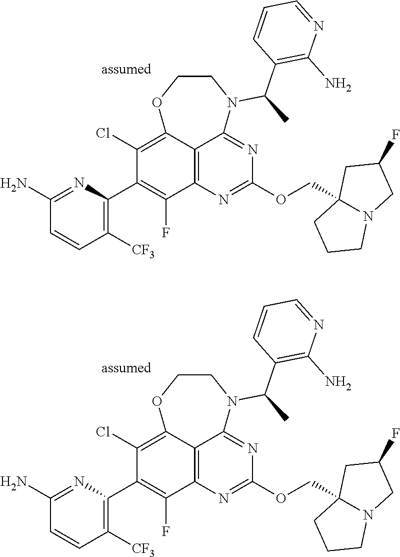

### Synthetic Route

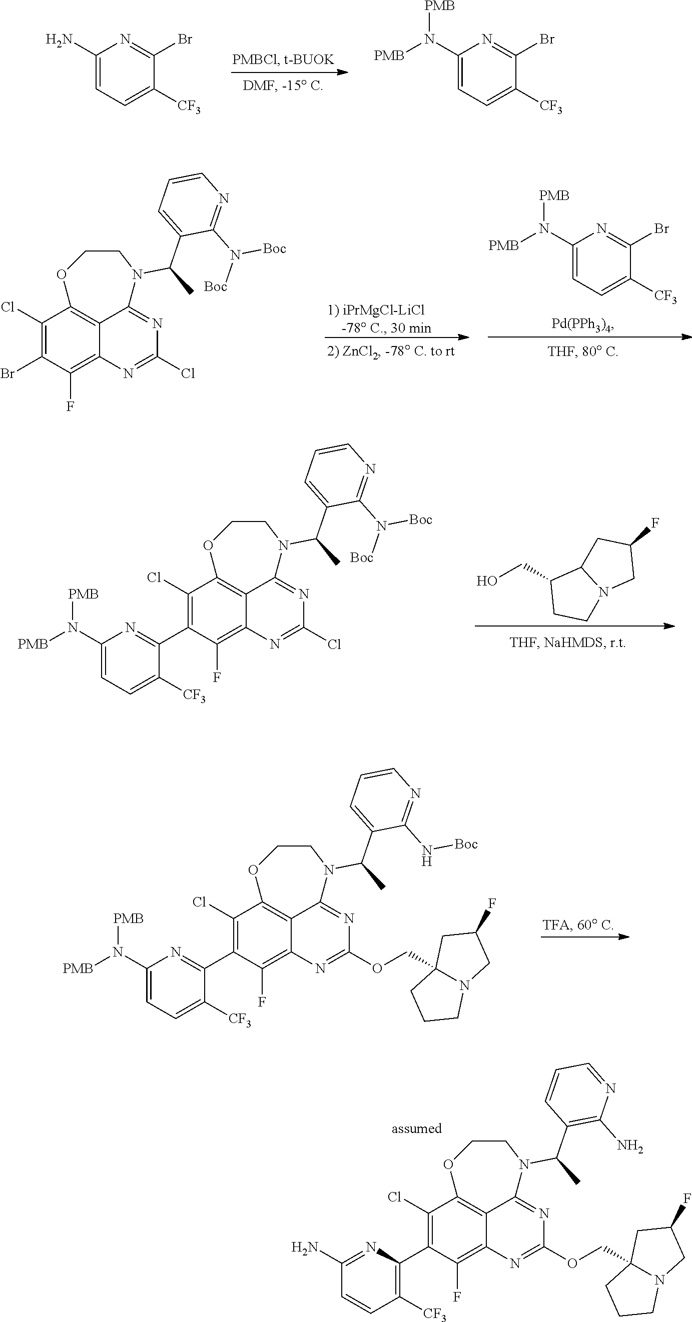

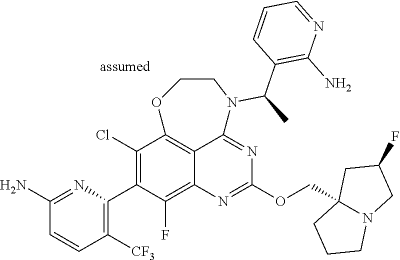

Step 1: 6-bromo-N,N-bis(4-methoxybenzyl)-5-(trifluoromethyl)pyridin-2-amine

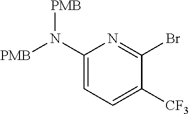

Under nitrogen, a solution of 6-bromo-5-(trifluoromethyl)pyridin-2-amine (1.80 g, 7.50 mmol) in N,N-dimethylformamide (18 mL) was added sodium hydride (0.9 g, 22.5 mmol, 60% dispersion in mineral oil) at 0° C. and stirred for 30 min at room temperature. Then 4-methoxybenzylchloride (2.93 g, 18.8 mmol) was dropwise added at 0° C. and stirred at room temperature for 1 hour. After completion, the reaction was quenched with saturated ammonium chloride solution, diluted with water, extracted with water, washed with water, dried over anhydrous sodium sulfate and concentrated under vacuum. The residue was purified by flash chromatography on silica gel eluting with dichloromethane/ethyl acetate (5:1) to afford the title compound (3.50 g, 7.40 mmol, 98.7% yield) as a white solid. LCMS (ESI, m/z): 481.1 [M+H]⁺.

Step 2: tert-butyl (3-((1R)-1-(9-(6-(bis(4-methoxybenzyl)amino)-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)carbamate

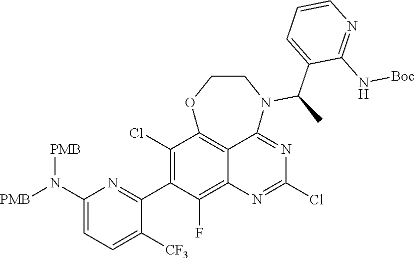

Under nitrogen, a solution of tert-butyl N-[3-[(1R)-1-(7-bromo-3,8-dichloro-6-fluoro-10-oxa-2,4,13-triazatricyclo[7.4.1.05,14]tetradeca-1,3,5,7,9(14)-pentaen-13-yl)ethyl]-2-pyridyl]-N-tert-butoxycarbonyl-carbamate (500.0 mg, 0.74 mmol) in tetrahydrofuran (15 mL) was added isopropyl magnesium chloride lithium chloride (1.15 mL, 1.49 mmol, 1.3M in tetrahydrofuran) at −78° C. and stirred at −78° C. for 60 min. Then zinc chloride (0.75 mL, 1.5 mmol, 1.0M in 2-MeTHF) was added at −78° C., stirred at −78° C. for 5 min and stirred at 40° C. for 30 min. To the mixture was added tetrakis(triphenylphosphine)palladium (250.0 mg, 0.22 mmol), 6-bromo-N,N-bis[(4-methoxyphenyl)methyl]-4-methyl-5-(trifluoromethyl)pyridin-2-amine (480.0 mg, 0.97 mmol) in tetrahydrofuran (15 mL) and stirred at 80° C. for 35 min. After completion, the reaction was quenched by saturated ammonium chloride solution, diluted with dichloromethane, washed with saturated sodium chloride and dried over anhydrous sodium sulfate and concentrated under vacuum. The residue was purified by flash chromatography on silica gel eluting with dichloromethane/ethyl acetate (5:1) to afford the title compound (200.0 mg, 0.22 mmol, 30.07% yield) as a yellow solid. LCMS (ESI, m/z): 894.2 [M+H]⁺.

Step 3: tert-butyl (3-((1R)-1-(9-(6-(bis(4-methoxybenzyl)amino)-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate

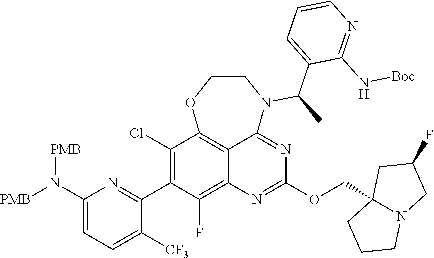

Under nitrogen, a solution of ((2R,7aS)-2-fluorotetrahydro-1H-pyrrolizin-7a(5H)-yl)methanol (99.6 mg, 0.63 mmol) in tetrahydrofuran (5 mL) was added sodium bis(trimethylsilyl)amide (0.78 mL, 0.78 mmol, 1M in tetrahydrofuran) at 0° C. and stirred for 20 min at 25° C. Then the reaction solution was transferred to a solution of tert-butyl N-[3-[(1R)-1-[7-[6-[bis[(4-methoxyphenyl)methyl]amino]-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]carbamate (140.0 mg, 0.16 mmol) in tetrahydrofuran (5 mL) and stirred for 40 min at 25° C. After completion, the reaction was diluted with ethyl acetate, washed with saturated brine, dried over anhydrous sodium sulfate and concentrated under vacuum. The residue was purified by flash chromatography on silica gel eluting with dichloromethane/methanol (10:1) to afford the title compound (80.0 mg, 0.08 mmol, 50.3% yield) as a yellow solid. LCMS (ESI, m/z): 1017.4 [M+H]⁺.

Step 4: 6-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-5-(trifluoromethyl)pyridin-2-amine & 6-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-5-(trifluoromethyl)pyridin-2-amine

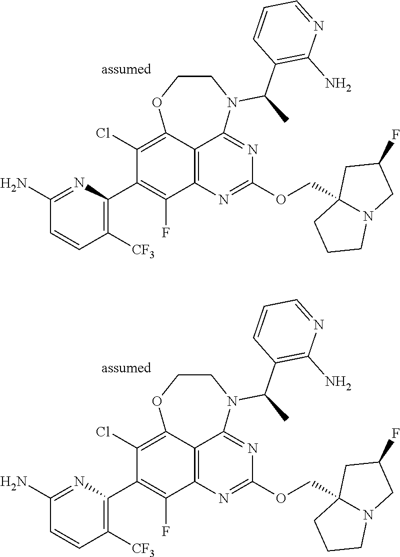

A solution of tert-butyl (3-((1R)-1-(9-(6-(bis(4-methoxybenzyl)amino)-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (70.0 mg, 0.07 mmol) in 2,2,2-trifluoroacetic acid (5 mL) was stirred at 60° C. for 3 hours. After completion, the reaction was concentrated under vacuum. The residue was diluted with ethyl acetate, adjusted PH=7.0 with saturated sodium carbonate solution, washed with saturated brine, dried over anhydrous sodium sulfate and concentrated under vacuum to afford 60 mg product. The product was purified and separated by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 41% B to 62% B in 10 min, 62% B; Wave Length: 254/220 nm; RT1(min): 9.22; This resulted in 6-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-5-(trifluoromethyl)pyridin-2-amine (2.5 mg, 0.0037 mmol, 5.3% yield) and 6-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-5-(trifluoromethyl)pyridin-2-amine (4.2 mg, 0.006 mmol, 9.0% yield).

Example 108a: ¹H NMR (400 MHz, Methanol-d₄, ppm) δ 7.95 (dd, J=5.1, 1.7 Hz, 1H), 7.81 (d, J=8.9 Hz, 1H), 7.75 (dd, J=7.6, 1.7 Hz, 1H), 6.78 (dd, J=7.5, 5.1 Hz, 1H), 6.72 (d, J=8.8 Hz, 1H), 6.53 (q, J=6.9 Hz, 1H), 5.32 (d, J=53.2 Hz, 1H), 4.54-4.40 (m, 1H), 4.35-4.17 (m, 3H), 3.73 (dd, J=15.9, 7.3, 1.6 Hz, 1H), 3.51 (dd, J=14.8, 5.8, 1.5 Hz, 1H), 3.28-3.19 (m, 2H), 3.08-2.98 (m, 1H), 2.45-2.30 (m, 1H), 2.28 (d, J=4.6 Hz, 1H), 2.26-2.11 (m, 2H), 2.07-1.83 (m, 3H), 1.66 (d, J=7.0 Hz, 3H). LCMS (ESI, m/z): 677.2 [M+H]⁺.

Example 108b: ¹H NMR (400 MHz, Methanol-d₄, ppm) δ 7.95 (dd, J=5.1, 1.7 Hz, 1H), 7.81 (d, J=8.9 Hz, 1H), 7.75 (dd, J=7.6, 1.7 Hz, 1H), 6.78 (dd, J=7.5, 5.1 Hz, 1H), 6.72 (d, J=8.8 Hz, 1H), 6.53 (q, J=6.9 Hz, 1H), 5.32 (d, J=53.2 Hz, 1H), 4.54-4.40 (m, 1H), 4.35-4.17 (m, 3H), 3.73 (dd, J=15.9, 7.3, 1.6 Hz, 1H), 3.51 (dd, J=14.8, 5.8, 1.5 Hz, 1H), 3.28-3.19 (m, 2H), 3.08-2.98 (m, 1H), 2.45-2.30 (m, 1H), 2.28 (d, J=4.6 Hz, 1H), 2.26-2.11 (m, 2H), 2.07-1.83 (m, 3H), 1.66 (d, J=7.0 Hz, 3H). LCMS (ESI, m/z): 677.2 [M+H]⁺.

Example 109: 6-((R)-4-((R)-1-(2-aminopyridin-3-yl)ethyl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

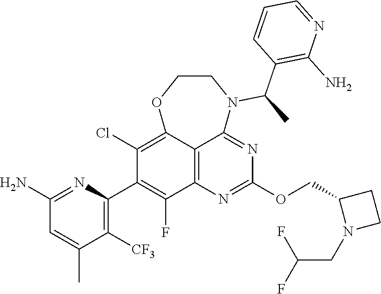

### Synthetic Route

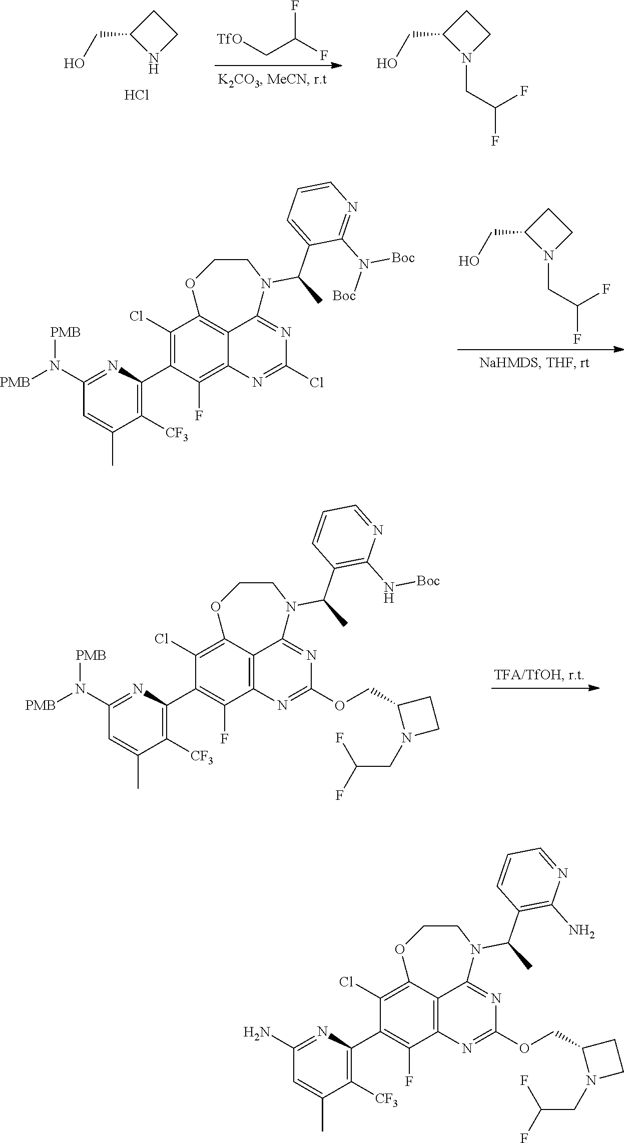

Step 1: (S)-(I-(2,2-difluoroethyl)azetidin-2-yl)methanol

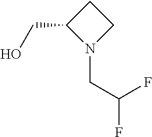

A solution of (S)-azetidin-2-ylmethanol hydrochloride (2.00 g, 22.96 mmol) and potassium carbonate (9.50 g, 68.87 mmol) in acetonitrile (20 mL). Then 2,2-difluoroethyl trifluoromethanesulfonate (5.41 g, 25.25 mmol) was added and stirred at 30° C. for overnight. After completion, the solvent was filtered and concentrated under vacuum. The residue was purified by flash chromatography on silica gel eluting with methanol/dichloromethane (1:20) to afford (S)-(1-(2,2-difluoroethyl)azetidin-2-yl)methanol (550.0 mg, 3.2 mmol, 14.3% yield) as a yellow oil. LC-MS: (ESI, m/z): 152.1 [M+H]⁺

Step 2: tert-butyl (3-((R)-1-((R)-9-(6-(bis(4-methoxybenzyl)amino)-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyridin-2-yl)carbamate

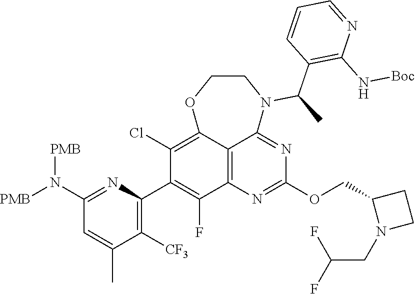

A solution of (S)-(1-(2,2-difluoroethyl)azetidin-2-yl)methanol (469.2 mg, 3.1 mmol) in tetrahydrofuran (16 mL) was added sodium bis(trimethylsilyl)amide [1.0 M in tetrahydrofuran] (3.5 mL, 3.5 mmol) and stirred at 25° C. for 30 minutes. Then tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (desired atropisomer) (783.0 mg, 0.7 mmol) was added and stirred at 25° C. for 1 hour. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with ethyl acetate/dichloromethane (1:6) to afford the title compound (485.0 mg, 0.4 mmol, 58% yield) as a yellow solid. LC-MS: (ESI, m/z): 1023.4 [M+H]⁺

Step 3: 6-((R)-4-((R)-1-(2-aminopyridin-3-yl)ethyl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

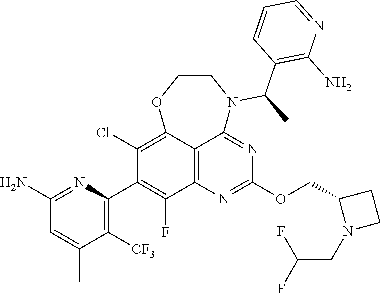

A solution of tert-butyl (3-((R)-1-((R)-9-(6-(bis(4-methoxybenzyl)amino)-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyridin-2-yl)carbamate (480.0 mg, 0.4 mmol) in trifluoromethanesulfonic acid (0.5 mL) and 2,2,2-trifluoroacetic acid (5 mL) was stirred at 25° C. for 1 hour. After completion, the solvent was concentrated under vacuum. The reaction mixture was diluted with ethyl acetate. The reaction mixture was adjusted to pH=8 with sodium carbonate aqueous solution. The organic layer was washed with water, dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min: Gradient: 17% B to 45% B in 9 min, 45% B; Wave Length: 254/220 nm; RT1(min): 8.5 to afford the title compound (260.0 mg, 0.3 mmol, 81.1% yield). LC-MS: (ESI, m/z): 683.3 [M+H]⁺

Example 109: ¹H NMR (300 MHz, DMSO-d₆, ppm) δ 7.98 (dd, J=4.9, 1.7 Hz, 1H), 7.64 (dd, J=7.4, 1.7 Hz, 1H), 6.82 (s, 2H), 6.67 (dd, J=7.5, 4.9 Hz, 1H), 6.48 (s, 1H), 6.23 (q, J=6.8 Hz, 1H), 5.96 (tdd, J=56.1, 5.2, 3.3 Hz, 1H), 5.67 (s, 2H), 4.49-4.18 (m, 4H), 3.71-3.56 (m, 2H), 3.42-3.35 (m, 2H), 3.17-2.95 (m, 2H), 2.85-2.67 (m, 1H), 2.36 (s, 3H), 2.13-1.99 (m, 2H), 1.56 (d, J=6.7 Hz, 3H).

Example 110a & 110b: 6-((R)-4-((R)-1-(2-aminopyridin-3-yl)ethyl)-8-chloro-10-fluoro-2-(2-methoxy-2-methylpropoxy)-5,6-dihydro-4H-[1,4]oxazepino[5,6,7-de]quinazolin-9-yl)-4-methyl-5-(trifluoromethyl)pyridin-2-amine, 6-((S)-4-((R)-1-(2-aminopyridin-3-yl)ethyl)-8-chloro-10-fluoro-2-(2-methoxy-2-methylpropoxy)-5,6-dihydro-4H-[1,4]oxazepino[5,6,7-de]quinazolin-9-yl)-4-methyl-5-(trifluoromethyl)pyridin-2-amine

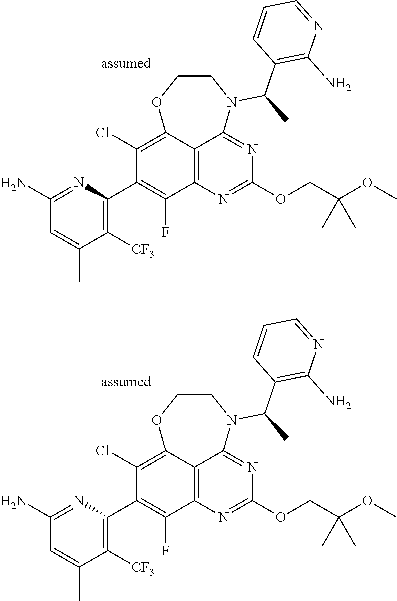

### Synthetic Route:

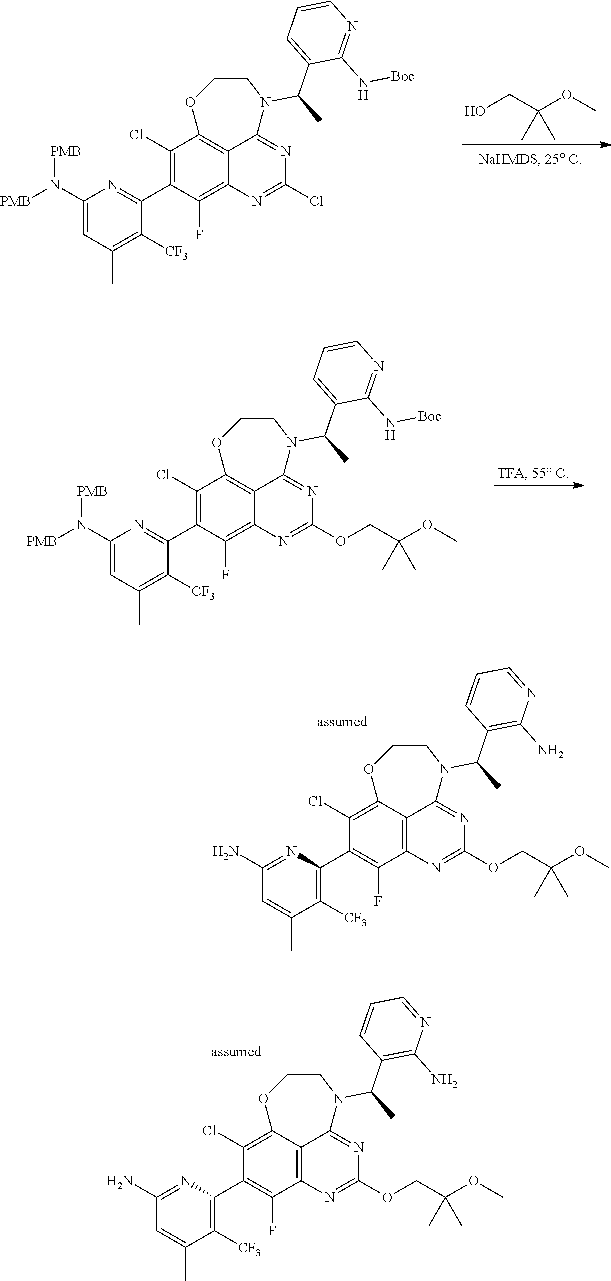

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-(2-methoxy-2-methylpropoxy)-5,6-dihydro-4H-[1,4]oxazepino[5,6,7-de]quinazolin-4-yl)ethyl)pyridin-2-yl)carbamate

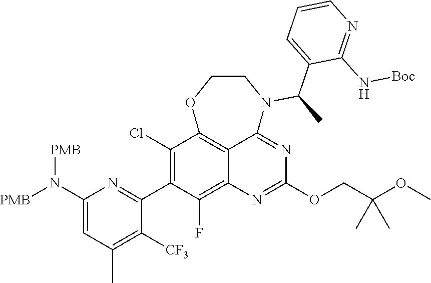

Under nitrogen, a solution of 2-methoxy-2-methylpropan-1-ol (37.0 mg, 0.36 mmol) in tetrahydrofuran (0.5 mL) was added sodium bis(trimethylsilyl)amide (0.55 mL, 0.55 mmol, 1 M in tetrahydrofuran) at 0° C. and stirred for 15 minutes at 25° C. Then tert-butyl (3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)carbamate (250.0 mg, 0.28 mmol) was added and stirred at 25° C. for 0.5 hour. After completion, the resulting solution was diluted with ethyl acetate and washed with water. The organic layer was washed with brine and dried over anhydrous sodium sulfate and concentrated under vacuum. The residue was purified by flash chromatography on silica gel eluting with petroleum ether/ethyl acetate (2:3) to afford the title compound (211.0 mg, 0.22 mmol, 78.6% yield) as a white solid. LC-MS: (ESI, m/z): 976.4 [M+H]⁺

Step 2: 6-((R)-4-((R)-1-(2-aminopyridin-3-yl)ethyl)-8-chloro-10-fluoro-2-(2-methoxy-2-methylpropoxy)-5,6-dihydro-4H-[1,4]oxazepino[5,6,7-de]quinazolin-9-yl)-4-methyl-5-(trifluoromethyl)pyridin-2-amine, 6-((S)-4-((R)-1-(2-aminopyridin-3-yl)ethyl)-8-chloro-10-fluoro-2-(2-methoxy-2-methylpropoxy)-5,6-dihydro-4H-[1,4]oxazepino[5,6,7-de]quinazolin-9-yl)-4-methyl-5-(trifluoromethyl)pyridin-2-amine

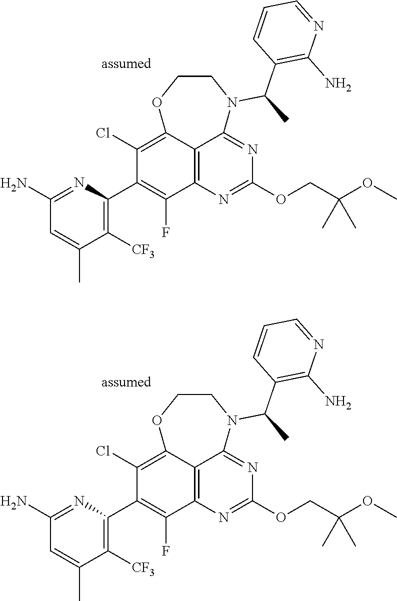

A solution of trifluoroacetic acid (9 mL, 116.82 mmol) and tert-butyl (3-((1R)-1-(9-(6-(bis(4-methoxybenzyl)amino)-4-methyl-3-(trifluoromethyl)pyridin-2-yl)-8-chloro-10-fluoro-2-(2-methoxy-2-methylpropoxy)-5,6-dihydro-4H-[1,4]oxazepino[5,6,7-de]quinazolin-4-yl)ethyl)pyridin-2-yl)carbamate (200.0 mg, 0.20 mmol) in dichloromethane (3 mL) was stirred at 55° C. for 16 hours. After completion, the mixture was concentrated under vacuum. The residue was diluted with dichloromethane, adjusted to pH=7 by saturated sodium bicarbonate solution, washed with brine. The organic layer was dried over anhydrous sodium sulfate and concentrated under vacuum. The crude product was purified by Prep-HPLC with the following conditions: Column: Xselect CSH C18 OBD Column 30*150 mm 5 μm, n; Mobile Phase A: Water (0.1% FA), Mobile Phase B: ACN: Flow rate: 60 mL/min; Gradient: 23% B to 39% B in 10 min, 39% B; Wave Length: 254/220 nm; RT1(min): 7 to afford 6-((R)-4-((R)-1-(2-aminopyridin-3-yl)ethyl)-8-chloro-10-fluoro-2-(2-methoxy-2-methylpropoxy)-5,6-dihydro-4H-[1,4]oxazepino[5,6,7-de]quinazolin-9-yl)-4-methyl-5-(trifluoromethyl)pyridin-2-amine (22.0 mg, 0.035 mmol, 16.9% yield) and 6-((S)-4-((R)-1-(2-aminopyridin-3-yl)ethyl)-8-chloro-10-fluoro-2-(2-methoxy-2-methylpropoxy)-5,6-dihydro-4H-[1,4]oxazepino[5,6,7-de]quinazolin-9-yl)-4-methyl-5-(trifluoromethyl)pyridin-2-amine (8.3 mg, 0.013 mmol, 6.4% yield).

Example 110a: ¹H NMR (300 MHz, DMSO-da, ppm) δ 7.97 (dd, J=5.1, 1.6 Hz, 1H), 7.70 (d, J=7.5 Hz, 1H), 6.81 (s, 2H), 6.71 (dd, J=7.5, 5.1 Hz, 1H), 6.47 (s, 1H), 6.23 (d, J=6.8 Hz, 1H), 6.05 (s, 2H), 4.45 (dd, J=11.8, 6.1 Hz, 1H), 4.36-4.25 (m, 1H), 4.25 (s, 2H), 3.68 (dd, J=15.6, 6.8 Hz, 1H), 3.51-3.36 (m, 1H), 3.16 (s, 3H), 2.36 (s, 3H), 1.58 (d, J=6.8 Hz, 3H), 1.21 (d, J=3.6 Hz, 6H). LC-MS: (ESI, m/z): 636.2 [M+H]⁺

Example 110b: ¹H NMR (300 MHz, DMSO-dg, ppm) δ 7.95 (dd, J=4.9, 1.7 Hz, 1H), 7.62 (d, J=7.5 Hz, 1H), 6.81 (s, 2H), 6.65 (dd, J=7.5, 4.9 Hz, 1H), 6.47 (s, 1H), 6.27 (q, J=6.9 Hz, 1H), 5.77 (s, 2H), 4.51 (dd, J=12.0, 6.5 Hz, 1H), 4.38-4.18 (m, 3H), 3.71 (dd, J=15.5, 6.4 Hz, 1H), 3.44-3.35 (m, 1H), 3.16 (s, 3H), 2.36 (s, 3H), 1.58 (d, J=6.8 Hz, 3H), 1.21 (d, J=3.4 Hz, 6H). LC-MS: (ESI, m/z): 636.2 [M+H]⁺

Example 111a & 111b: 6-((R)-4-((R)-1-(2-aminopyridin-3-yl)ethyl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((S)-4-((R)-1-(2-aminopyridin-3-yl)ethyl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-9-yl)-4-methyl-5-(trifluoromethyl)pyridin-2-amine

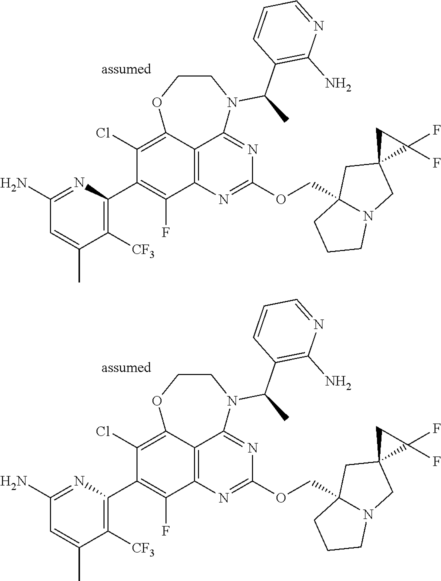

### Synthetic Route

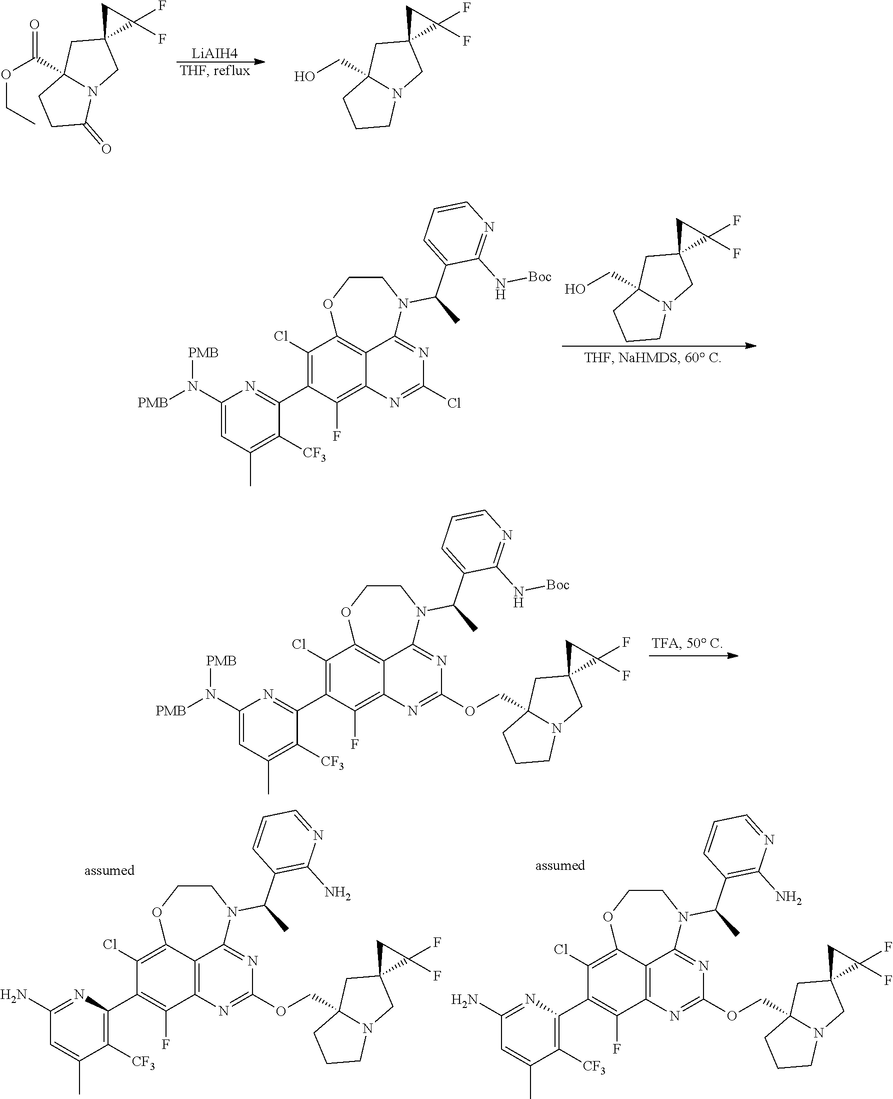

Step 1: ((1R,7a′S)-2,2-difluorodihydro-1′H,3′H-spiro[cyclopropane-1,2′-pyrrolizin]-7a′(5′H)-yl)methanol

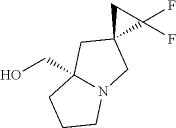 [SMILES: OC[C@@]12CCCN1C[C@]1(CC1(F)F)C2]

Under nitrogen, a solution of ethyl (1R,7a′S)-2,2-difluoro-5′-oxodihydro-1′H,3′H-spiro[cyclopropane-1,2′-pyrrolizine]-7a′(5′H)-carboxylate (400.0 mg, 1.54 mmol) in tetrahydrofuran (5 mL) was added lithium aluminium hydride (211.1 mg, 5.55 mmol) at 0° C. and stirred for 40 min at 65° C. After completion, the reaction was quenched with sodium sulfate decahydrate, diluted with tetrahydrofuran and filtered. Then the filter was concentrated under vacuum to afforded crude product (300 mg, crude). LCMS (ESI, m/z): 204.1 [M+H]⁺.

Step 2: tert-butyl (3-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyridin-2-yl)carbamate

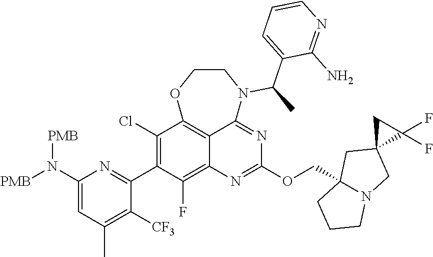

Under nitrogen, a solution of ((1R,7a′S)-2,2-difluorodihydro-1′H,3′H-spiro[cyclopropane-1,2′-pyrrolizin]-7a′(5′H)-yl)methanol (201.3 mg, 0.99 mmol) in tetrahydrofuran (15 mL) was added sodium bis(trimethylsilyl)amide (1.16 ml, 1.16 mmol, 1M in tetrahydrofuran) at room temperature for 20 min. Then the reaction solution was added into the solution of tert-butyl (3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)carbamate (300.00 mg, 0.33 mmol) in tetrahydrofuran (15 mL) was added and stirred at 60° C. for 1 hour. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with dichloromethane/methanol to afford the title compound (150.0 mg, 0.14 mmol, 38.6% yield). LCMS (ESI, m/z): 1075.4 [M+H]⁺.

Step 3: 6-((R)-4-((R)-1-(2-aminopyridin-3-yl)ethyl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((S)-4-((R)-1-(2-aminopyridin-3-yl)ethyl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-9-yl)-4-methyl-5-(trifluoromethyl)pyridin-2-amine

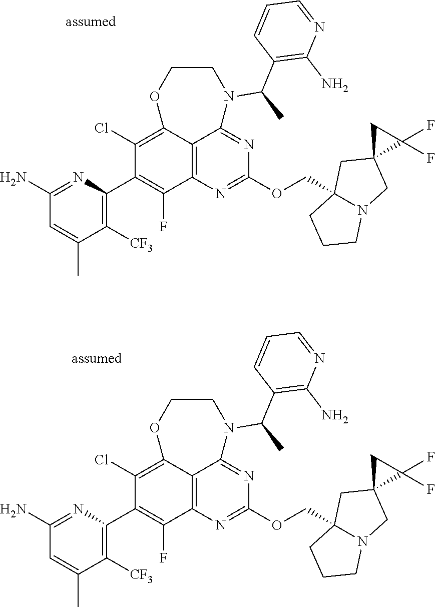

A solution of tert-butyl (3-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyridin-2-yl)carbamate (150.0 mg, 0.14 mmol) in 2,2,2-trifluoroacetic acid (10 mL) was stirred at 50° C. for 2 hours. After completion, the reaction solution was concentrated under vacuum. The residue was purified by Prep-HPLC with the following Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 41% B to 63% B in 8 min, 63% B; Wave Length: 254/220 nm; RT1(min): 8 to afford 6-((R)-4-((R)-1-(2-aminopyridin-3-yl)ethyl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-9-yl)-4-methyl-5-(trifluoromethyl)pyridin-2-amine (26.3 mg, 0.035 mmol, 25.3% yield) and 6-((S)-4-((R)-1-(2-aminopyridin-3-yl)ethyl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-9-yl)-4-methyl-5-(trifluoromethyl)pyridin-2-amine (12.2 mg, 0.016 mmol, 11.8% yield).

Example 111a: ¹H NMR (300 MHz, DMSO-d₆, ppm) δ 7.97 (dd, J=4.9, 1.7 Hz, 1H), 7.63 (d, J=7.4 Hz, 1H), 6.80 (s, 2H), 6.66 (dd, J=7.5, 4.9 Hz, 1H), 6.47 (s, 1H), 6.25 (q, J=7.0 Hz, 1H), 5.73 (s, 2H), 4.43 (dd, J=11.9, 6.0 Hz, 1H), 4.32-4.07 (m, 3H), 3.64 (dd, J=15.6, 6.9 Hz, 1H), 3.39 (d, J=6.7 Hz, 1H), 3.17-2.95 (m, 2H), 2.71 (d, J=11.9 Hz, 1H), 2.58-2.52 (m, 1H), 2.36 (s, 3H), 2.14-1.95 (m, 2H), 1.90 (d, J=13.4 Hz, 1H), 1.86-1.67 (m, 2H), 1.67-1.39 (m, 6H). LCMS (ESI, m/z): 735.2 [M+H]⁺.

Example 111b: ¹H NMR (300 MHz, DMSO-d₆, ppm) δ 7.95 (dd, J=4.9, 1.7 Hz, 1H), 7.63 (d, J=7.4 Hz, 1H), 6.80 (s, 2H), 6.65 (dd, J=7.5, 4.9 Hz, 1H), 6.47 (s, 1H), 6.28 (q, J=6.9 Hz, 1H), 5.80 (s, 2H), 4.50 (dd, J=12.2, 6.6 Hz, 1H), 4.38-4.28 (m, 1H), 4.27-4.07 (m, 2H), 3.70 (dd, J=15.6, 6.4 Hz, 1H), 3.27 (d, J=20.9 Hz, 1H), 3.14-2.94 (m, 2H), 2.71 (d, J=11.7 Hz, 1H), 2.60-2.51 (m, 1H), 2.36 (s, 3H), 2.12-1.94 (m, 2H), 1.89 (d, J=13.4 Hz, 1H), 1.86-1.69 (m, 2H), 1.68-1.35 (m, 6H). LCMS (ESI, m/z): 735.2 [M+H]⁺.

Example 112: 6-((R)-4-((R)-1-(2-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

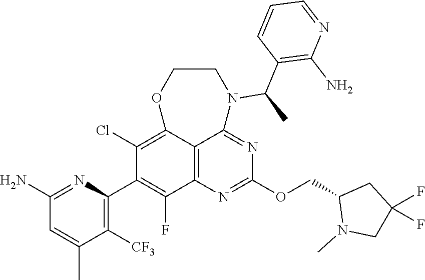

### Synthetic Route

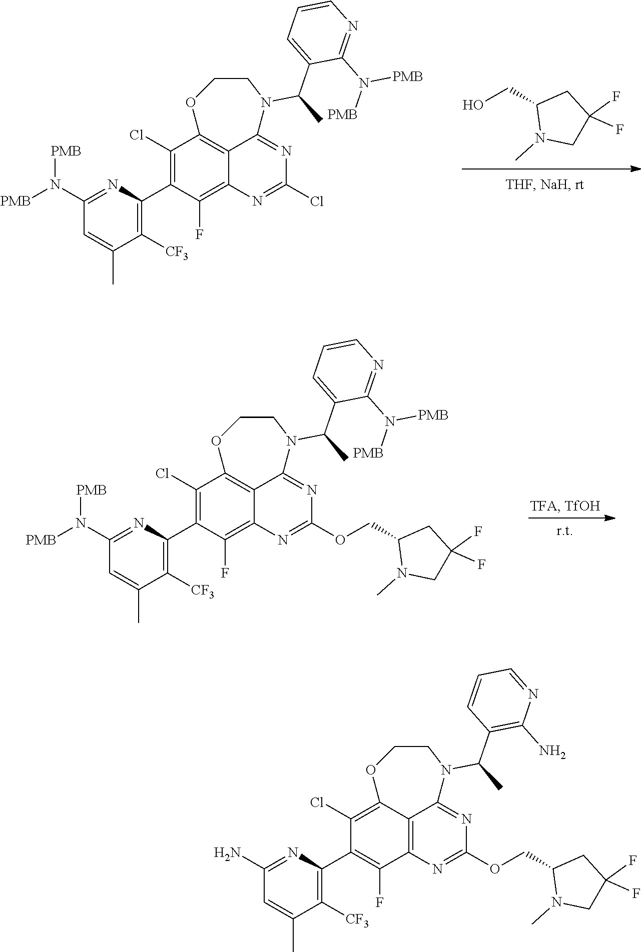

Step 1: (R)-6-(4-((R)-1-(2-(bis(4-methoxybenzyl)amino)pyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

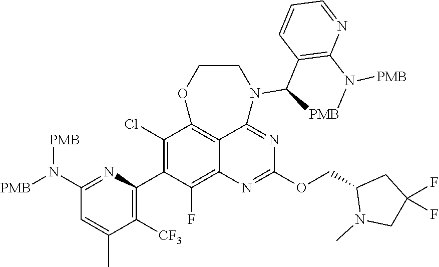

A solution of (S)-(4,4-difluoro-1-methylpyrrolidin-2-yl)methanol (1.90 g, 12.6 mmol) in tetrahydrofuran (30 mL) was added sodium hydride (1.26 g, 31.5 mmol, 60% purity) and stirred at 0° C. for 15 minutes. Then the reaction solution was transferred into a solution of (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (11.00 g, 10.5 mmol) in tetrahydrofuran (100 mL) was added and stirred at 25° C. for 6 hours. After completion, the reaction was quenched with saturated ammonium chloride solution and concentrated under vacuum to remove tetrahydrofuran. The reaction mixture was diluted with ethyl acetate, washed with water, dried over anhydrous sodium sulfate and concentrated under vacuum. The residue was purified by flash chromatography on silica gel eluting with ethyl acetate/petroleum ether (1:8) to afford the title compound (11.00 g, 9.4 mmol, 90.1% yield) as a white solid. LC-MS: (ESI, m/z): 1163.1 [M+H]⁺

Step 2: 6-((R)-4-((R)-1-(2-aminopyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

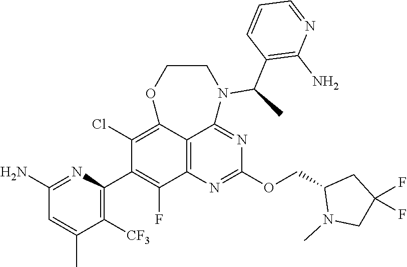

A solution of (R)-6-(4-((R)-1-(2-(bis(4-methoxybenzyl)amino)pyridin-3-yl)ethyl)-8-chloro-2-(((S)-4,4-difluoro-1-methylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (11.00 g, 9.4 mmol) in trifluoroacetic acid (80 mL) and trifluoromethanesulfonic acid (8 mL) was stirred at 25° C. for 1 hour. After completion, the reaction mixture was concentrated under vacuum. The residue was diluted with dichloromethane, adjusted to pH=7 with saturated sodium bicarbonate solution, washed with water and dried over anhydrous sodium sulfate and concentrated under vacuum. The residue was purified by flash chromatography on reversed-phase C18 column water (0.1% ammonium bicarbonate)(A)/acetonitrile(B) Gradient: 5% B to 42% B in 30 min to afford the title compound (3.41 g, 4.90 mmol, 52.8% yield). LC-MS: (ESI, m/z): 683.1 [M+H]⁺

Example 112: ¹H NMR (300 MHz, DMSO-d₆, ppm) δ 7.98 (dd, J=4.9, 1.7 Hz, 1H), 7.64 (dd, J=7.5, 1.8 Hz, 1H), 6.81 (s, 2H), 6.67 (dd, J=7.5, 4.9 Hz, 1H), 6.48 (s, 1H), 6.24 (q, J=6.7 Hz, 1H), 5.67 (s, 2H), 4.57-4.18 (m, 4H), 3.77-3.54 (m, 1H), 3.44-3.34 (m, 2H), 3.04-2.87 (m, 1H), 2.76-2.55 (m, 2H), 2.41-2.30 (m, 6H), 2.29-1.98 (m, 1H), 1.57 (d, J=6.8 Hz, 3H).

Example 113a & 113b: 1-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)isoquinolin-3-amine & 1-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)isoquinolin-3-amine

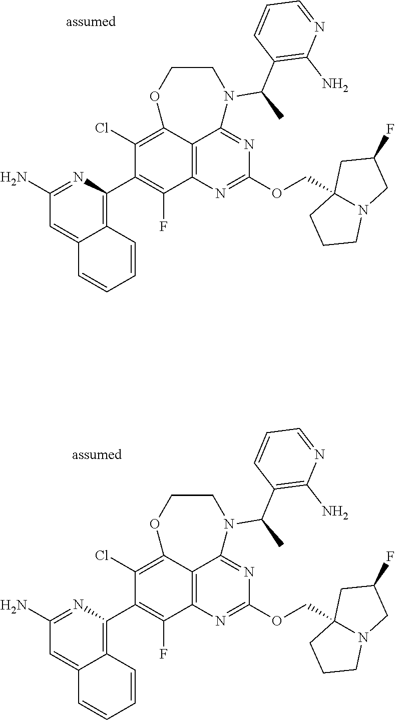

### Synthetic Route

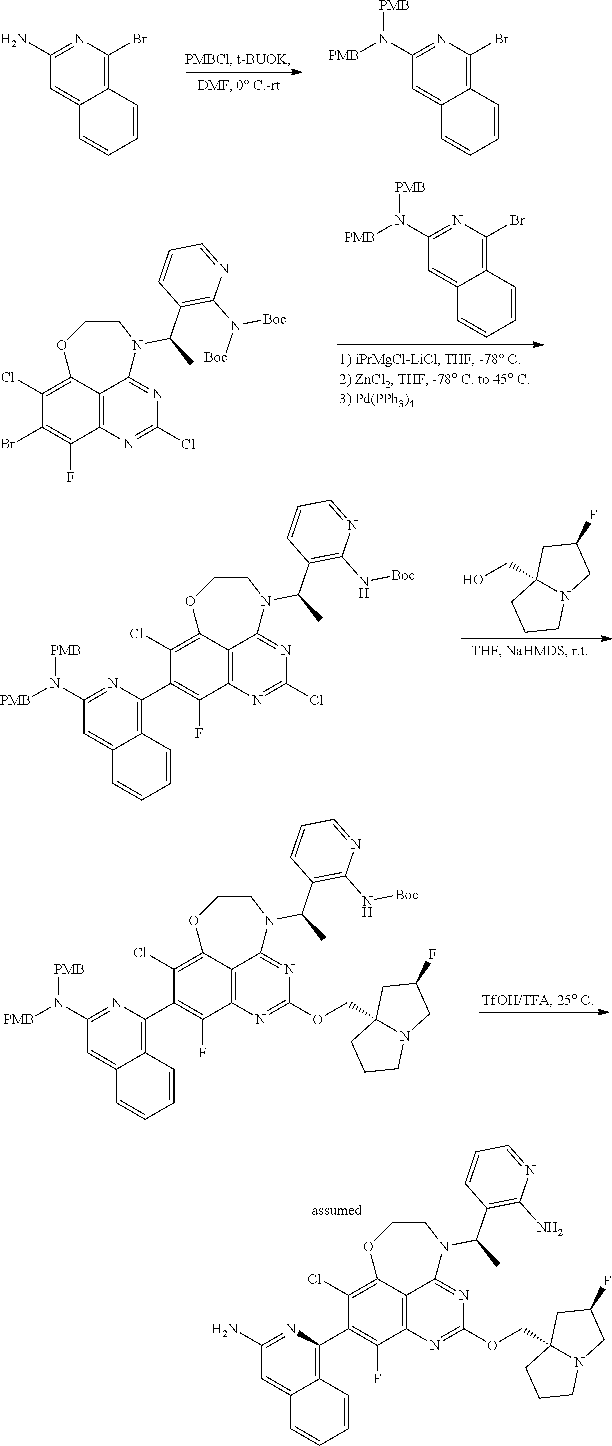

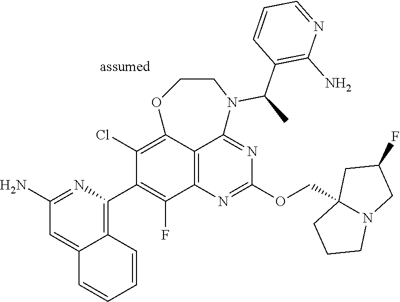

Step 1: 1-bromo-N,N-bis(4-methoxybenzyl)isoquinolin-3-amine

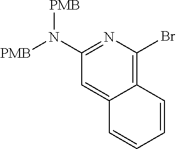

A solution of 1-bromoisoquinolin-3-amine (500.0 mg, 2.2 mmol) and potassium tert-butoxide (754.5 mg, 6.7 mmol) in N,N-dimethylformamide (6 mL) was stirred at 25° C. for 5 minutes. Then 4-methoxybenzyl chloride (0.7 mL, 5.6 mmol) was added and stirred at 25° C. for 2 hours. After completion, the reaction mixture was diluted with water. The resulting solution was extracted with ethyl acetate and the organic layers were combined. The organic layers were washed with water again. The organic layer was dried over anhydrous sodium sulfate and concentrated under vacuum. The residue was purified by flash chromatography on silica gel eluting with dichloromethane/petroleum ether (1:10) to afford the title compound (300.0 mg, 0.6 mmol, 28.9% yield). LC-MS: (ESI, m/z): 463.1 [M+H]⁺.

Step 2: tert-butyl N-[3-[(1R)-1-[7-[3-[bis[(4-methoxyphenyl)methyl]amino]-1-isoquinolyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]carbamate

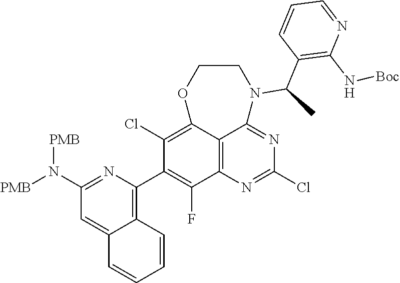

Under nitrogen, a solution of tert-butyl N-[3-[(1R)-1-(7-bromo-3,8-dichloro-6-fluoro-10-oxa-2,4,13-triazatricyclo[7.4.1.05,14]tetradeca-1,3,5(14),6,8-pentaen-13-yl)ethyl]-2-pyridyl]-N-tert-butoxycarbonyl-carbamate (200.0 mg, 0.3 mmol) in tetrahydrofuran (5 mL) was added isopropylmagnesium chloride lithium chloride complex (0.4 mL, 0.5 mmol, 1.3 M in tetrahydrofuran) and stirred at −78° C. for 10 minutes. Then zinc chloride (0.3 mL, 0.6 mmol, 2 M in tetrahydrofuran) was added at −78° C. and stirred at 45° C. for 40 minutes. Then 1-bromo-N,N-bis[(4-methoxyphenyl)methyl]isoquinolin-3-amine (151.4 mg, 0.3 mmol) and tetrakis(triphenylphosphine)palladium (102.9 mg, 0.1 mmol) was added and stirred at 80° C. for 15 minutes. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with ethyl acetate/petroleum ether (1:8) to afford the title compound (200.0 mg, 0.2 mmol, 76.8% yield) as a color solid. LC-MS: (ESI, m/z): 976.3 [M+H]⁺

Step 3: tert-butyl (3-((1R)-1-(9-(3-(bis(4-methoxybenzyl)amino)isoquinolin-1-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate

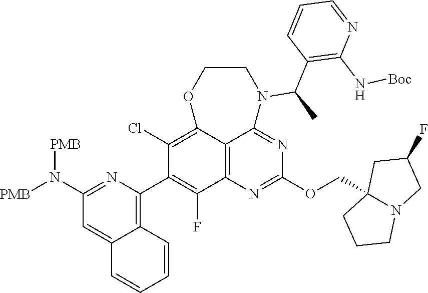

Under nitrogen, to a solution of ((2R,7aS)-2-fluorotetrahydro-1H-pyrrolizin-7a(5H)-yl)methanol (108.9 mg, 0.7 mmol) in tetrahydrofuran (3 mL) was added sodium bis(trimethylsilyl)amide (0.9 mL, 0.9 mmol, 1M in tetrahydrofuran) and stirred at 25° C. for 10 minutes. The mixture was transferred into a mixture of tert-butyl N-[3-[(1R)-1-[7-[3-[bis[(4-methoxyphenyl)methyl]amino]-1-isoquinolyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]carbamate (200.0 mg, 0.2 mmol) in tetrahydrofuran (3 mL) and stirred at 25° C. for 1 hour. After completion, the reaction was quenched with saturated ammonium chloride solution. The reaction mixture was diluted with ethyl acetate, washed with water, dried over anhydrous sodium sulfate and concentrated under vacuum. The residue was purified by flash chromatography on silica gel eluting with methanol/dichloromethane (1:20) to afford the title compound (100.0 mg, 0.1 mmol, 43.9% yield). LC-MS: (ESI, m/z): 1098.9 [M+H]⁺.

Step 4

### 1-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)isoquinolin-3-amine & 1-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)isoquinolin-3-amine

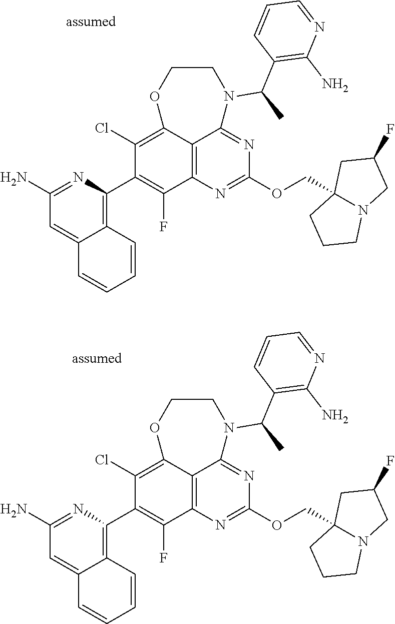

A solution of tert-butyl (3-((1R)-1-(9-(3-(bis(4-methoxybenzyl)amino)isoquinolin-1-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (100.0 mg, 0.1 mmol) in trifluoroacetic acid (1 mL) and trifluoromethanesulfonic acid (0.1 mL) was stirred at 25° C. for 15 minutes. After completion, the reaction mixture was concentrated under vacuum, diluted with dichloromethane, adjusted to pH=7 with saturated sodium bicarbonate solution, washed with water, dried over anhydrous sodium sulfate and concentrated under vacuum. The residue was purified by flash chromatography on reversed-phase C18 column: water (0.1% ammonium bicarbonate)(A)/acetonitrile(B) Gradient: 5% B to 60% B in 30 min to afford the product. The product was separated by Prep-HPLC with the following conditions (Column: XBridge Prep C18 OBD Column, 30*100 mm, 5 μm; Mobile Phase A: Water (10 mmol/L NH₄HCO₃), Mobile Phase B: ACN; Flow rate: 60 mL/min; Gradient: 35% B to 55% B in 10 min, 55% B; Wave Length: 254/220) to afford 1-((R)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)isoquinolin-3-amine (15.7 mg, 0.02 mmol, 23.8% yield) and 1-((S)-4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)isoquinolin-3-amine (15.5 mg, 0.02 mmol, 23.5% yield).

Example 113a: ¹H NMR (300 MHz, DMSO-d₆, ppm) δ 7.98 (dd, J=4.9, 1.7 Hz, 1H), 7.69-7.57 (m, 2H), 7.46 (dd, J=8.3, 6.6, Hz, 1H), 7.22 (d, J=8.5 Hz, 1H), 7.10-7.00 (m, 1H), 6.74 (d, J=0.9 Hz, 1H), 6.67 (dd, J=7.4, 4.9 Hz, 1H), 6.30 (q, J=6.9 Hz, 1H), 6.11 (s, 2H), 5.83 (s, 2H), 5.28 (d, J=54.4 Hz, 1H), 4.53 (dd, J=12.0, 6.5 Hz, 1H), 4.31 (dd, J=12.0, 6.5 Hz, 1H), 4.09 (s, 2H), 3.75 (dd, J=15.6, 6.6 Hz, 1H), 3.53-3.37 (m, 1H), 3.24-3.00 (m, 2H), 2.99 (s, 1H), 2.88-2.69 (m, 1H), 2.26-2.11 (m, 1H), 2.10-1.90 (m, 2H), 1.90-1.70 (m, 3H), 1.60 (d, J=6.8 Hz, 3H). LC-MS: (ESI, m/z): 659.4 [M+H]⁺.

Example 113b: ¹H NMR (300 MHz, DMSO-d₆, ppm) δ 7.98 (dd, J=4.9, 1.7 Hz, 1H), 7.69-7.57 (m, 2H), 7.46 (dd, J=8.3, 6.6, Hz, 1H), 7.22 (d, J=8.5 Hz, 1H), 7.10-7.00 (m, 1H), 6.74 (d, J=0.9 Hz, 1H), 6.67 (dd, J=7.4, 4.9 Hz, 1H), 6.30 (q, J=6.9 Hz, 1H), 6.10 (s, 2H), 5.78 (s, 2H), 5.28 (d, J=54.3 Hz, 1H), 4.49 (dd, J=12.0, 6.2 Hz, 1H), 4.30 (dd, J=11.9, 6.6 Hz, 1H), 4.09 (q, J=10.4 Hz, 2H), 3.71 (dd, J=15.7, 6.6 Hz, 1H), 3.43 (dd, J=15.8, 6.4 Hz, 1H), 3.17-3.01 (m, 2H), 2.99 (s, 1H), 2.88-2.70 (m, 1H), 2.31-2.12 (m, 1H), 2.11-1.95 (m, 2H), 1.92-1.67 (m, 3H), 1.59 (d, J=6.8 Hz, 3H). LC-MS: (ESI, m/z): 659.3 [M+H]⁺.

Example 114a & 114b: 6-((R)-4-((R)-1-(2-aminopyridin-3-yl)ethyl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-9-yl)-4-methyl-5-(trifluoromethyl)pyridin-2-amine

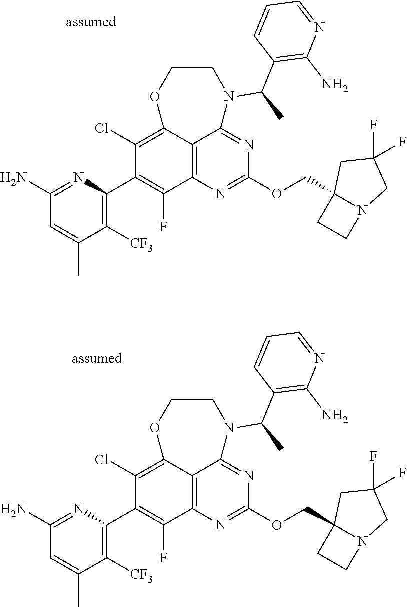

### Synthetic Route

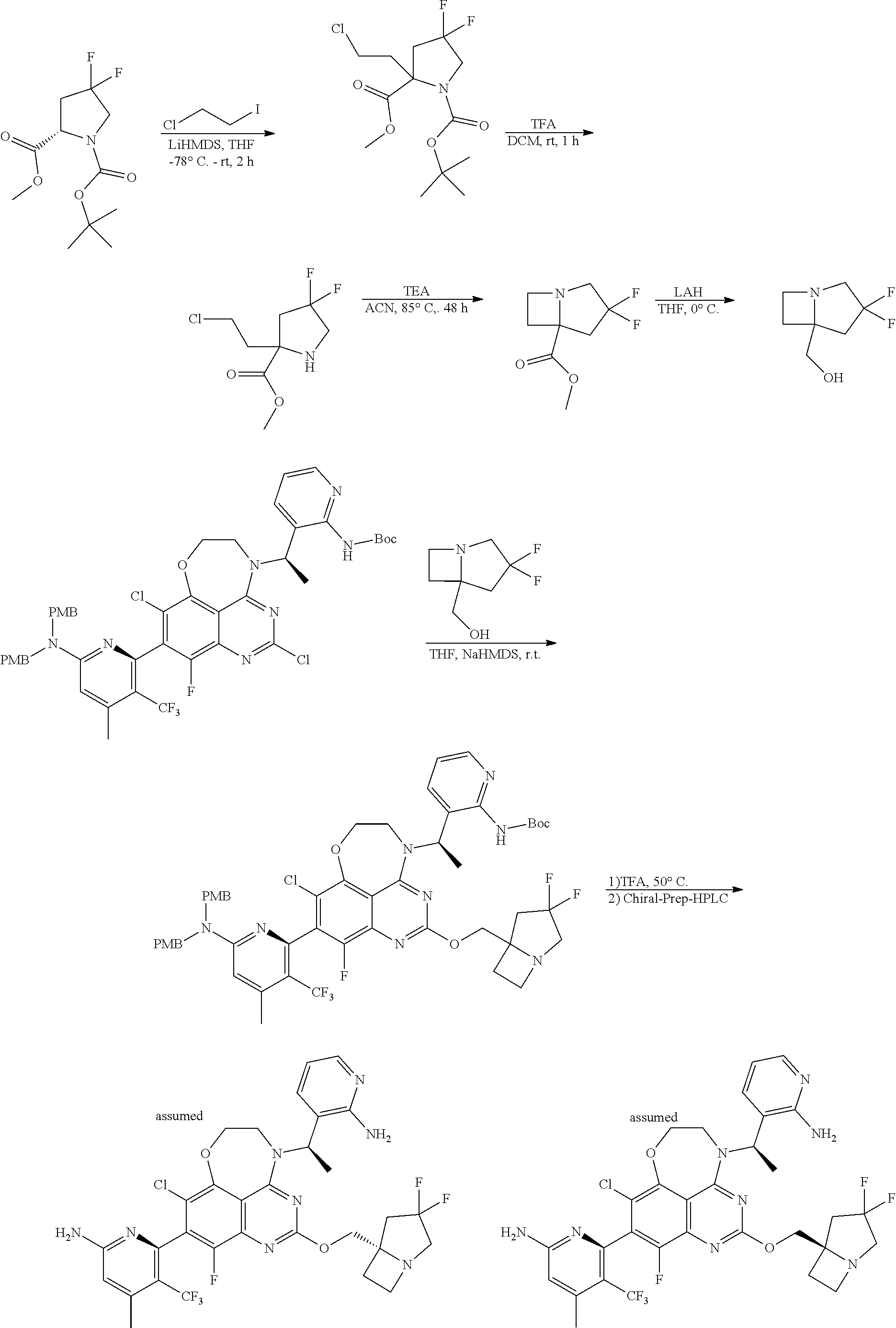

Step 1: 1-(tert-butyl) 2-methyl 2-(2-chloroethyl)-4,4-difluoropyrrolidine-1,2-dicarboxylate

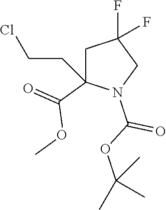 [SMILES: COC(=O)C1(CCCl)CC(F)(F)CN1C(=O)OC(C)(C)C]

A solution of 1-(tert-butyl) 2-methyl (S)-4,4-difluoropyrrolidine-1,2-dicarboxylate (3.00 g, 11.31 mmol) in tetrahydrofuran (30 mL) was dropwise lithium bis(trimethylsilyl)amide (33.9 mL, 33.93 mmol, 1M in THF) and stirred at −78° C. for 1 hour. Then 1-chloro-2-iodoethane (4.30 g, 22.62 mmol) was dropwise added and stirred at room temperature for 2 hours. After completion, the solvent was concentrated under vacuum. The residue was purified by flash chromatography on silica gel eluting with petroleum ether/ethyl acetate (1:9) to afford 1-(tert-butyl) 2-methyl 2-(2-chloroethyl)-4,4-difluoropyrrolidine-1,2-dicarboxylate (2.00 g, 6.10 mmol, 54% yield) as an oil. LCMS (ESI, m/z): 328.1 [M+H]⁺.

Step 2: methyl 2-(2-chloroethyl)-4,4-difluoropyrrolidine-2-carboxylate

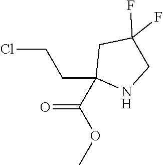

A solution of 1-(tert-butyl) 2-methyl 2-(2-chloroethyl)-4,4-difluoropyrrolidine-1,2-dicarboxylate (2.00 g, 6.10 mmol) in 2,2,2-trifluoroacetic acid (10 mL) and dichloromethane (10 mL) was stirred at 25° C. for 1 hour. After completion, the solvent was concentrated under vacuum to afford 1.9 g crude as black oil which was directly used without purification.

Step 3: methyl 3,3-difluoro-1-azabicyclo[3.2.0]heptane-5-carboxylate

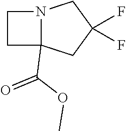

A solution of methyl 2-(2-chloroethyl)-4,4-difluoro-pyrrolidine-2-carboxylate (621.0 mg, 2.73 mmol) and triethylamine (1.02 g, 10.92 mmol) in acetonitrile (18 mL) was stirred at 85° C. for 48 hours. After completion, the solvent was concentrated under vacuum. The residue was purified by flash chromatography on silica gel eluting with petroleum ether/ethyl acetate (2:3) to afford methyl 3,3-difluoro-1-azabicyclo[3.2.0]heptane-5-carboxylate (450.0 mg, 2.35 mmol, 86.3% yield) as a red oil.

LCMS (ESI, m/z): 192.2 [M+H]⁺.

Step 4: (3,3-difluoro-1-azabicyclo[3.2.0]heptan-5-yl)methanol

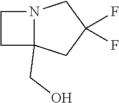 [SMILES: OCC12CCN1CC(F)(F)C2]

A solution of methyl 3,3-difluoro-1-azabicyclo[3.2.0]heptane-5-carboxylate (440.0 mg, 2.3 mmol) in tetrahydrofuran (12 mL) was dropwise added aluminum lithium hydride (2.7 mL, 6.9 mmol, 2.5M in tetrahydrofuran) and stirred at 25° C. for 0.5 hours. After completion, the reaction was quenched by sodium sulfate decahydrate and diluted with tetrahydrofuran. Then the reaction solution was filtered and the filtrate was blowed out by N₂ to afford (3,3-difluoro-1-azabicyclo[3.2.0]heptan-5-yl)methanol (350 mg, crude).

LCMS (ESI, m/z): 164.2 [M+H]⁺.

Step 5: tert-butyl (3-((1R)-1-(9-((R)-6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridin-2-yl)carbamate

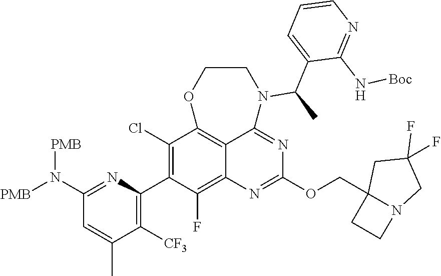

Under nitrogen, a solution of (3,3-difluoro-1-azabicyclo[3.2.0]heptan-5-yl)methanol (485.7 mg, 2.98 mmol) in tetrahydrofuran (6 mL) was dropwise added sodium bis(trimethylsilyl)amide (3.98 mL, 3.98 mmol, 1M in tetrahydrofuran) at 25° C. and stirred for 20 min at 25° C. Then the reaction solution was transferred into the solution of tert-butyl (3-((R)-1-(9-((R)-6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)carbamate (900.0 mg, 0.99 mmol) in tetrahydrofuran (12 mL) and stirred for 2 h at 25° C. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with petroleum ether/ethyl acetate (1:3) to afford the title compound (780.0 mg, 0.54 mmol, 41.1% yield) as a yellow solid. LCMS (ESI, m/z): 1035.3 [M+H]⁺.

Step 6: 6-((R)-4-((R)-1-(2-aminopyridin-3-yl)ethyl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-9-yl)-4-methyl-5-(trifluoromethyl)pyridin-2-amine

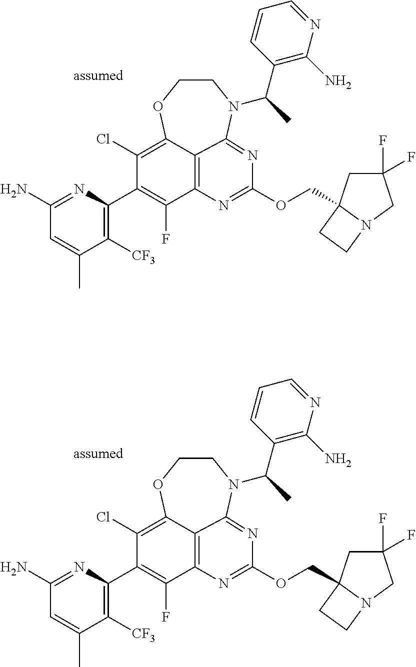

A solution of tert-butyl (3-((1R)-1-(9-((R)-6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyridin-2-yl)carbamate (1.00 g, 0.97 mmol) in trifluoromethanesulfonic acid (0.5 ml) and 1,1,2-trichlorotrifluoroethane (5 mL) was stirred at 25° C. for 0.5 hours. After completion, the reaction was concentrated under vacuum. The residue was diluted with ethyl acetate, adjusted PH=7.0 with saturated sodium carbonate solution, washed with saturated brine, dried over anhydrous sodium sulfate and concentrated under vacuum. The crude product was purified by flash chromatography on silica gel. The product was separated by Pre-Chiral-HPLC with conditions: Column: CHIRAL ART Cellulose-SB, 2*25 cm, 5 μm; Mobile Phase A: Hex (0.5% 2M NH3-MeOH)-HPLC, Mobile Phase B: EtOH-HPLC; Flow rate: 20 mL/min; Gradient: 50% B to 50% B in 22 min; Wave Length: 220/254 nm; RT1(min): 8.841; RT2(min): 15.431; Sample Solvent: EtOH-HPLC; Injection Volume: 0.75 mL: Number Of Runs: 6. This afforded to 6-((R)-4-((R)-1-(2-aminopyridin-3-yl)ethyl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-9-yl)-4-methyl-5-(trifluoromethyl)pyridin-2-amine (42.1 mg, 0.06 mmol, 6.3% yield) and 6-((R)-4-((R)-1-(2-aminopyridin-3-yl)ethyl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-9-yl)-4-methyl-5-(trifluoromethyl)pyridin-2-amine (38.0 mg, 0.05 mmol, 5.6% yield).

Example 114a: ¹H NMR (300 MHz, DMSO-da, ppm) δ 7.97 (dd, J=4.9, 1.7 Hz, 1H), 7.63 (dd, J=7.6, 1.8 Hz, 1H), 6.82 (s, 2H), 6.66 (dd, J=7.5, 4.9 Hz, 1H), 6.47 (s, 1H), 6.25 (q, J=6.8 Hz, 1H), 5.69 (s, 2H), 4.53-4.41 (m, 1H), 4.41-4.32 (m, 2H), 4.32-4.16 (m, 1H), 3.65 (dd, J=15.7, 6.7 Hz, 1H), 3.59-3.46 (m, 1H), 3.46-3.37 (m, 1H), 3.27-3.15 (m, 1H), 3.15-2.98 (m, 2H), 2.76-2.54 (m, 2H), 2.46-2.19 (m, 5H), 1.57 (d, J=6.8 Hz, 3H). LCMS (ESI, m/z): 695.30 [M+H]⁺. Chiral HPLC: Column: CHIRAL Cellulose-SB, 4.6*100 mm, 3 μm; Mobile Phase: Hex (0.1% DEA):EtOH=70:30; Flow: 1.0 mL/min: Retention time: 4.160 min (First peak).

Example 114b: ¹H NMR (300 MHz, DMSO-dg, ppm) δ 7.97 (dd, J=4.9, 1.7 Hz, 1H), 7.63 (dd, J=7.5, 1.7 Hz, 1H), 6.82 (s, 2H), 6.66 (dd, J=7.5, 4.9 Hz, 1H), 6.47 (s, 1H), 6.25 (q, J=6.8 Hz, 1H), 5.74 (s, 2H), 4.54-4.38 (m, 1H), 4.36 (s, 2H), 4.33-4.17 (m, 1H), 3.66 (dd, J=15.6, 6.8 Hz, 1H), 3.60-3.46 (m, 1H), 3.46-3.39 (m, 1H), 3.22-2.98 (m, 3H), 2.79-2.54 (m, 2H), 2.44-2.23 (m, 5H), 1.57 (d, J=6.8 Hz, 3H). LCMS (ESI, m/z): 695.30 [M+H]⁺. Chiral HPLC: Column: CHIRAL Cellulose-SB, 4.6*100 mm, 3 μm; Mobile Phase: Hex (0.1% DEA):EtOH=70:30; Flow: 1.0 mL/min; Retention time: 5.378 min (Second peak).

Example 115: 6-((R)-4-((R)-1-(2-aminopyridin-3-yl)ethyl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

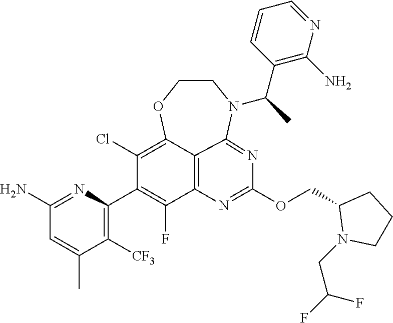

### Synthetic Route

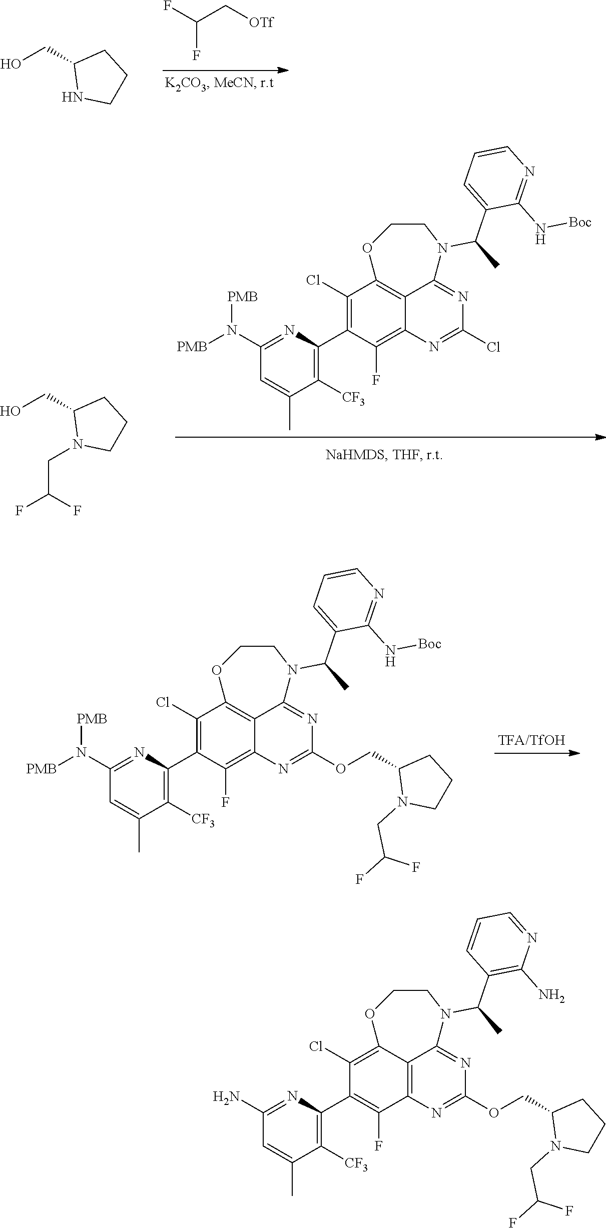

Step 1: (S)-(1-(2,2-difluoroethyl)pyrrolidin-2-yl)methanol

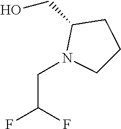 [SMILES: OC[C@@H]1CCCN1CC(F)F]

A solution of I-prolinol (0.49 mL, 4.94 mmol), 2,2-difluoroethyl trifluoromethanesulfonate (1.58 g, 7.41 mmol) and potassium carbonate (2.04 g, 14.83 mmol) in acetonitrile (12.5 mL) was stirred at 25° C. for 1 hour. After completion, the reaction was filtered. The filtration was concentrated under vacuum. The residue was purified by flash chromatography on silica gel eluting with petroleum ether/ethyl acetate (2:3) to afford (S)-(1-(2,2-difluoroethyl)pyrrolidin-2-yl)methanol (680.0 mg, 4.11 mmol, 83.3% yield) as a colorless oil. LC-MS: (ESI, m/z): 166.2 [M+H]

Step 2: tert-butyl (3-((R)-1-(9-((R)-6-(bis(4-methoxybenzyl)amino)-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyridin-2-yl)carbamate

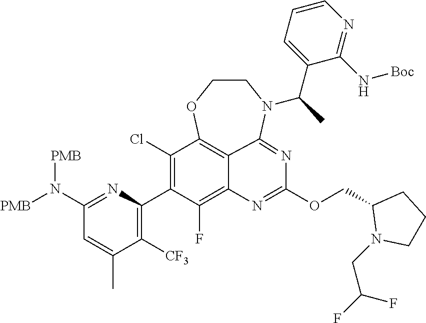

Under nitrogen, a solution of (S)-(1-(2,2-difluoroethyl)pyrrolidin-2-yl)methanol (49.12 mg, 0.30 mmol) in tetrahydrofuran (1 mL) was added sodium bis(trimethylsilyl)amide (0.6 mL, 0.60 mmol, 1M in tetrahydrofuran) and stirred for 10 min. Then the reaction solution was transferred to a solution of tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]carbamate (181.4 mg, 0.2 mmol) in in tetrahydrofuran (2 mL). The reaction was stirred for 1 hour. After completion, the reaction was quenched with saturated ammonium chloride solution. The reaction was diluted with ethyl acetate, washed with saturated brine, dried over anhydrous sodium sulfate and concentrated under vacuum to afford the title compound (90.0 mg, 0.07 mmol, 75.8% yield) as a yellow solid. LC-MS: (ESI, m/z): 1037.5[M+H]⁺

Step 3: 6-((R)-4-((R)-1-(2-aminopyridin-3-yl)ethyl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

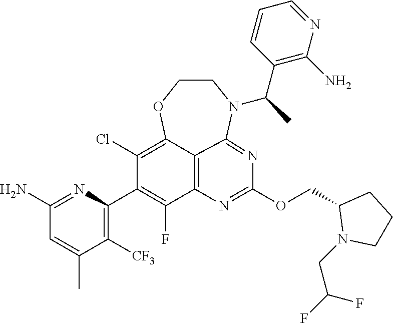

A solution of tert-butyl (3-((R)-1-(9-((R)-6-(bis(4-methoxybenzyl)amino)-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyridin-2-yl)carbamate (80.0 mg, 0.07 mmol) in trifluoromethanesulfonic acid (0.1 mL) and 1,1,2-trichlorotrifluoroethane (1 mL) was stirred at 25° C. for 0.5 hour. The solvent was concentrated under vacuum to afford the crude product. The crude product was purified by reverse phase chromatography and Pre-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 41% B to 62% B in 10 min, 62% B; Wave Length: 254/220 nm; RT1(min): 9.22; Number Of Runs: 0) to afforded the title compound (23.9 mg, 0.03 mmol, 48.6% yield). LCMS (ESI, m/z): 697.3 [M+H]⁺.

Example 115: ¹H NMR (300 MHz, DMSO-d₆, ppm) δ 7.96 (dd, J=4.9, 1.7 Hz, 1H), 7.63 (dd, J=7.5, 1.8 Hz, 1H), 6.82 (s, 2H), 6.66 (dd, J=7.5, 4.9 Hz, 1H), 6.53-6.41 (m, 1H), 6.34-6.22 (m, 1H), 6.22-5.82 (m, 1H), 5.67 (s, 2H), 4.52-4.31 (m, 2H), 4.30-4.18 (m, 1H), 4.13 (dd, J=10.8, 6.9 Hz, 1H), 3.63 (dd, J=15.5, 6.8 Hz, 1H), 3.51-3.36 (m, 1H), 3.34-3.18 (m, 2H), 3.18-2.95 (m, 1H), 2.94-2.68 (m, 1H), 2.47-2.38 (m, 1H), 2.36 (s, 3H), 2.07-1.83 (m, 1H), 1.83-1.60 (m, 2H), 1.55 (d, J=6.8 Hz, 3H), 1.23 (s, 1H).

Example 116: 6-((R)-4-((R)-1-(2-aminopyridin-3-yl)ethyl)-8-chloro-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

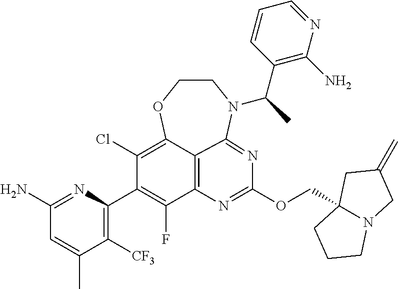

### Synthetic Route:

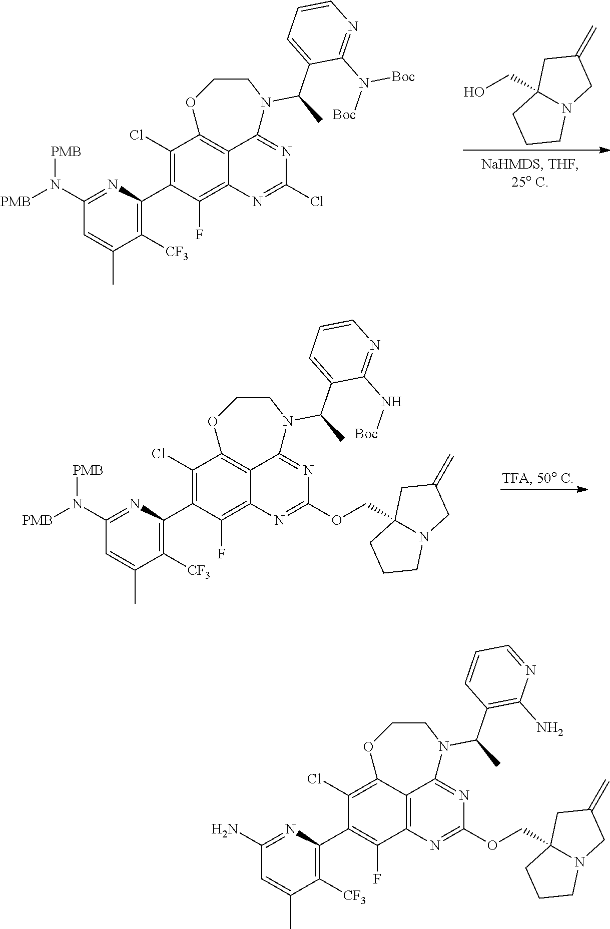

Step 1: tert-butyl (3-((R)-1-(9-((R)-6-(bis(4-methoxybenzyl)amino)-4-methyl-3-(trifluoromethyl)pyridin-2-yl)-8-chloro-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate

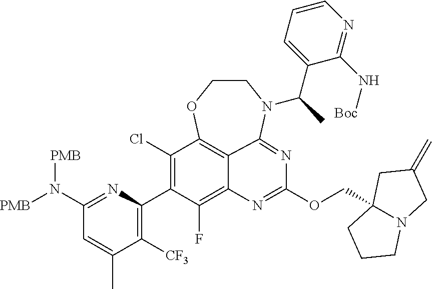

Under nitrogen, a solution of (S)-(2-methylenetetrahydro-1H-pyrrolizin-7a(5H)-yl)methanol (91.0 mg, 0.59 mmol) in tetrahydrofuran (2.8 mL) was added sodium bis(trimethylsilyl)amide (0.79 mL, 0.79 mmol, 1 M in tetrahydrofuran) at 25° C. and stirred for 15 minutes at 25° C. Then tert-butyl N-(3-((1R)-1-(7-(6-(bis((4-methoxyphenyl)methyl)amino)-4-methyl-3-(trifluoromethyl)-2-pyridyl)-3,8-dichloro-6-fluoro-10-oxa-2,4,13-triazatricyclo[7.4.1.05,14]tetradeca-1,3,5,7,9(14)-pentaen-13-yl)ethyl)-2-pyridyl)-N-tert-butoxycarbonyl-carbamate (desired atropisomer) (200.0 mg, 0.20 mmol) was added and stirred at 25° C. for 0.5 hour. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (9:1) to afford the title compound (75.0 mg, 0.073 mmol, 36.9% yield) as a yellow solid. LC-MS: (ESI, m/z): 1025.3 [M+H]⁺

Step 2: 6-((R)-4-((R)-1-(2-aminopyridin-3-yl)ethyl)-8-chloro-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

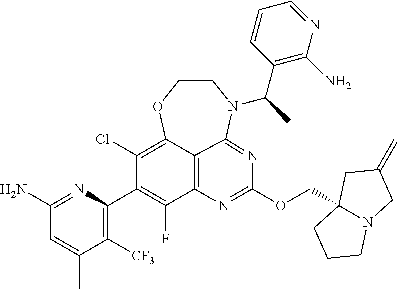

A solution of tert-butyl (3-((R)-1-(9-((R)-6-(bis(4-methoxybenzyl)amino)-4-methyl-3-(trifluoromethyl)pyridin-2-yl)-8-chloro-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (75.0 mg, 0.0700 mmol) in trifluoroacetic acid (1 mL, 12.98 mmol) was stirred at 50° C. for 2 hours. After completion, the mixture was concentrated under vacuum. The residue was diluted with dichloromethane, adjusted to pH=7 by saturated sodium bicarbonate solution, washed with brine. The organic layer was dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 30% B to 55% B in 9 min, 55% B; Wave Length: 254/220 nm; RT1(min): 9.6 to afford the title compound (8.0 mg, 0.012 mmol, 17.5% yield). LC-MS: (ESI, m/z): 685.4 [M+H]⁺

Example 116: ¹H NMR (300 MHz, Methanol-d₄, ppm) δ¹ 7.95 (dd, J=5.1, 1.6 Hz, 1H), 7.76 (d, J=9.0 Hz, 1H), 6.77 (dd, J=7.5, 5.1 Hz, 1H), 6.59 (s, 1H), 6.52 (q, J=6.8 Hz, 1H), 4.98 (s, 2H), 4.51-4.37 (m, 1H), 4.35-4.19 (m, 3H), 3.78-3.59 (m, 2H), 3.57-3.41 (m, 1H), 3.30-3.26 (m, 1H), 3.21-3.07 (m, 1H), 2.86-2.64 (m, 2H), 2.52-2.42 (m, 4H), 2.26-2.10 (m, 1H), 2.06-1.75 (m, 3H), 1.65 (d, J=6.9 Hz, 3H).

Example 117: 6-((R)-4-((R)-1-(2-aminopyridin-3-yl)ethyl)-8-chloro-10-fluoro-2-(((S)-1-(oxetan-3-yl)pyrrolidin-2-yl)methoxy)-5,6-dihydro-4H-[1,4]oxazepino[5,6,7-de]quinazolin-9-yl)-4-methyl-5-(trifluoromethyl)pyridin-2-amine

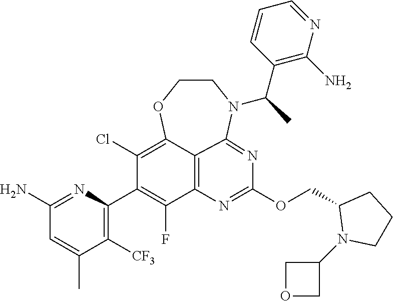

### Synthesis Route:

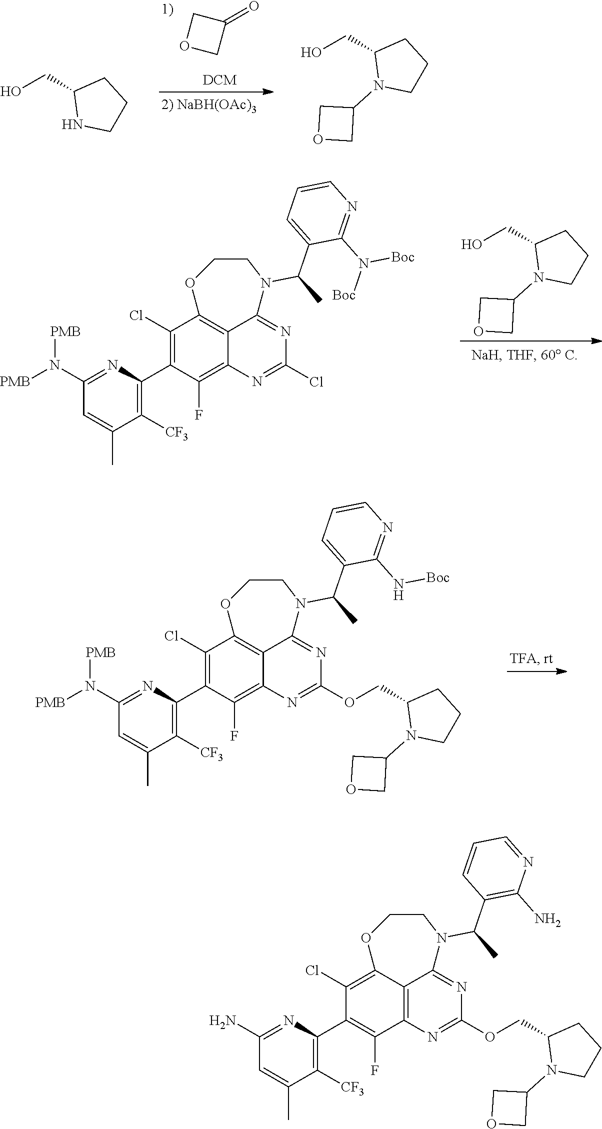

Step 1: (S)-(1-(oxetan-3-yl)pyrrolidin-2-yl)methanol

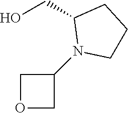 [SMILES: OC[C@@H]1CCCN1C1COC1]

A solution of L-prolinol (1.00 g, 9.89 mmol) and 3-oxetanone (1.00 g, 13.88 mmol) in dichloromethane (20 mL) was stirred at room temperature for 10 min. Then sodium triacetoxyborohydride (6.29 g, 29.68 mmol) was dropwise added and stirred at room temperature for 2 hours. After completion, the resulting solution was quenched with water, extracted with ethyl acetate. The organic layers were combined and concentrated under vacuum. The residue was purified by flash chromatography on silica gel eluting with dichloromethane/methanol to afford the title compound (400.0 mg, 25.7% yield) as colorless oil. LC-MS: (ESI, m/z): 158.1 [M+H]⁺

Step 2: tert-butyl (3-((R)-1-(9-((R)-6-(bis(4-methoxybenzyl)amino)-4-methyl-3-(trifluoromethyl)pyridin-2-yl)-8-chloro-10-fluoro-2-(((S)-1-(oxetan-3-yl)pyrrolidin-2-yl)methoxy)-5,6-dihydro-4H-[1,4]oxazepino[5,6,7-de]quinazolin-4-yl)ethyl)pyridin-2-yl)carbamate

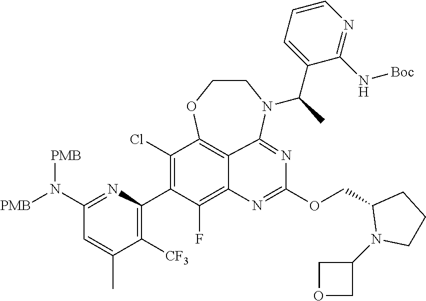

A solution of (S)-(1-(oxetan-3-yl)pyrrolidin-2-yl)methanol (47.1 mg, 0.3 mmol) in tetrahydrofuran (2 mL) was dropwise added sodium hydride (16.0 mg, 0.4 mmol, 60% purity) and stirred for 10 min at room temperature. Then the reaction solution was transferred into a solution of tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5(14),6,8-pentaen-13-yl]ethyl]-2-pyridyl]-N-tert-butoxycarbonyl-carbamate (desired atropisomer) (100.0 mg, 0.1 mmol) in tetrahydrofuran (3 mL) and stirred at 60° C. for 1 hour. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with dichloromethane/methanol (10:1) to afford the title compound (90.0 mg, 88.2% yield). LC-MS: (ESI, m/z): 1029.3 [M+H]⁺

Step 3: 6-((R)-4-((R)-1-(2-aminopyridin-3-yl)ethyl)-8-chloro-10-fluoro-2-(((S)-1-(oxetan-3-yl)pyrrolidin-2-yl)methoxy)-5,6-dihydro-4H-[1,4]oxazepino[5,6,7-de]quinazolin-9-yl)-4-methyl-5-(trifluoromethyl)pyridin-2-amine

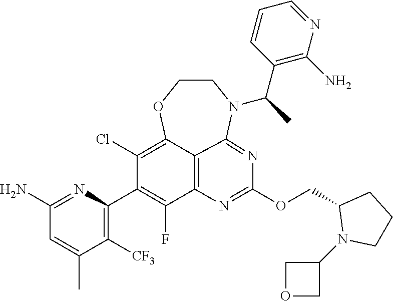

A solution of tert-butyl (3-((R)-1-(9-((R)-6-(bis(4-methoxybenzyl)amino)-4-methyl-3-(trifluoromethyl)pyridin-2-yl)-8-chloro-10-fluoro-2-(((S)-1-(oxetan-3-yl)pyrrolidin-2-yl)methoxy)-5,6-dihydro-4H-[1,4]oxazepino[5,6,7-de]quinazolin-4-yl)ethyl)pyridin-2-yl)carbamate (80.0 mg, 0.08 mmol) in trifluoroacetic acid (2 mL) was stirred at 25° C. for 2.6 days. After completion, the reaction solution was concentrated under vacuum, diluted with dichloromethane, washed with sodium carbonate solution, saturated brine, dried over anhydrous sodium sulfate and concentrated under vacuum. The residue was purified by flash chromatography on silica gel eluting with dichloromethane/methanol to afford the product. The product was further purified by Prep-HPLC with the following conditions (Column: XSelect CSH Fluoro Phenyl, 30*150 mm, 5 μm; Mobile Phase A: Water (10 mmol/L NH4HCO3), Mobile Phase B: ACN; Flow rate: 60 mL/min; Gradient: 33% B to 58% B in 9 min, 58% B: Wave Length: 220/254 nm; RT1(min): 7.92) to afford the title compound (16.7 mg, 0.024 mmol, 31.2% yield). LC-MS: (ESI, m/z): 689.3 [M+H]⁺

Example 117: ¹H NMR (300 MHz, DMSO-da, ppm) δ 7.97 (dd, J=4.9, 1.7 Hz, 1H), 7.63 (d, J=7.5 Hz, 1H), 6.80 (s, 2H), 6.67 (dd, J=7.5, 4.9 Hz, 1H), 6.47 (s, 1H), 6.24 (d, J=6.8 Hz, 1H), 5.67 (s, 2H), 4.67-4.47 (m, 4H), 4.42 (dd, J=12.0, 6.2 Hz, 1H), 4.34-4.16 (m, 2H), 4.08 (dd, J=10.8, 6.5 Hz, 1H), 4.03-3.82 (m, 1H), 3.63 (dd, J=15.6, 6.8 Hz, 1H), 3.42-3.35 (m, 1H), 3.03-2.83 (m, 2H), 2.43-2.32 (m, 4H), 2.00-1.82 (m, 1H), 1.81-1.61 (m, 3H), 1.56 (d, J=6.8 Hz, 3H).

Example 118: (6R)-6-(4-((R)-1-(2-aminopyridin-3-yl)ethyl)-8-chloro-10-fluoro-2-((3-methylene-1-azabicyclo[3.2.0]heptan-5-yl)methoxy)-5,6-dihydro-4H-[1,4]oxazepino[5,6,7-de]quinazolin-9-yl)-4-methyl-5-(trifluoromethyl)pyridin-2-amine

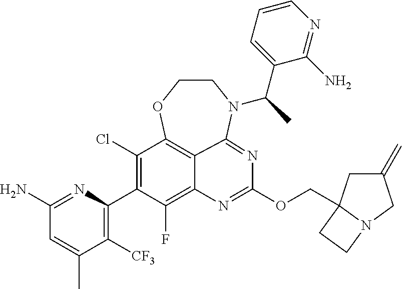

### Synthetic Route

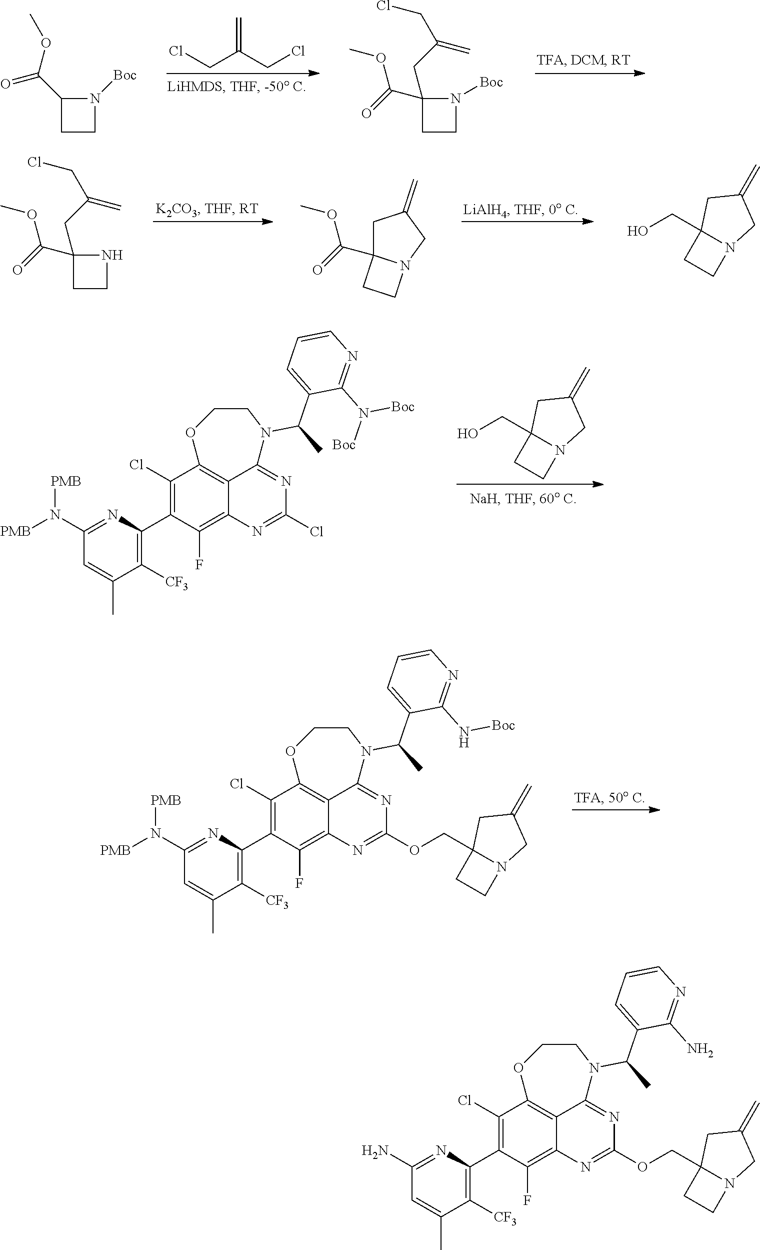

Step 1: 1-(tert-butyl) 2-methyl 2-(2-(chloromethyl)allyl)azetidine-1,2-dicarboxylate

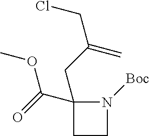 [SMILES: C=C(CCl)CC1(C(=O)OC)CCN1C(=O)OC(C)(C)C]

Under nitrogen, to a solution of 1-tert-butyl 2-methyl azetidine-1,2-dicarboxylate (9.00 g, 41.81 mmol) in tetrahydrofuran (65 mL) was added lithium bis(trimethylsilyl)amide (104 mL, 104 mmol, 1M) at −50° C. The mixture was stirred for 0.5 hour at −50° C. Then 3-chloro-2-(chloromethyl)prop-1-ene (20.00 g, 160 mmol) was added and stirred at −50° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with petroleum ether/ethyl acetate (4:1) to afford the title compound (1.30 g, 4.27 mmol, 10.2% yield) as a yellow oil. LC-MS: (ESI, m/z): 248.1 [M−55+H]⁺.

Step 2: methyl 2-(2-(chloromethyl)allyl)azetidine-2-carboxylate

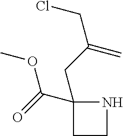

A solution of 1-(tert-butyl) 2-methyl 2-(2-(chloromethyl)allyl)azetidine-1,2-dicarboxylate (2.00 g, 6.58 mmol) in dichloromethane (20 mL) and trifluoroacetic acid (10 mL) was stirred at room temperature for 1 hour. After completion, the solvent was removed under vacuum. The crude product was used in the next step without purification. LC-MS: (ESI, m/z): 204.1 [M+H]⁺.

Step 3: methyl 3-methylene-1-azabicyclo[3.2.0]heptane-5-carboxylate

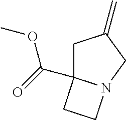

A solution of methyl 2-(2-(chloromethyl)allyl)azetidine-2-carboxylate (6.17 g, 30.29 mmol) and potassium carbonate (12.1 g, 87.68 mmol) in tetrahydrofuran (100 mL) was stirred at room temperature for 0.5 hour. After completion, the solvent was concentrated under vacuum. The resulting mixture was diluted with water and extracted with dichloromethane, the organic layers were combined, dried over anhydrous sodium sulfate and concentrated. The residue was purified by flash chromatography on silica gel eluting with dichloromethane/methanol (9:1) to afford methyl 3-methylene-1-azabicyclo[3.2.0]heptane-5-carboxylate (1.50 g, 29.6% yield) as a brown oil. LC-MS: (ESI, m/z): 168.1 [M+H]⁺.

Step 4: (3-methylene-1-azabicyclo[3.2.0]heptan-5-yl)methanol

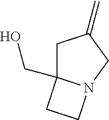

Under nitrogen, to a solution of methyl 3-methylene-1-azabicyclo[3.2.0]heptane-5-carboxylate (280 mg, 1.67 mmol) in tetrahydrofuran (4 mL) was added lithium aluminum hydride (130 mg, 3.42 mmol) at 0° C. The resulting solution was stirred for 2 hours at room temperature. After completion, the reaction was quenched with sodium sulfate decahydrate. The solids were filtered out. After filtration, the filtrate was concentrated under reduced pressure. The residue was purified by flash chromatography on silica gel eluting with dichloromethane/methanol (4:1) to afford (3-methylene-1-azabicyclo[3.2.0]heptan-5-yl)methanol (80.0 mg, 0.58 mmol, 34.3% yield) as a yellow solid. LC-MS: (ESI, m/z): 139.1 [M+H]⁺

Step 5: tert-butyl (3-((1R)-1-(9-((R)-6-(bis(4-methoxybenzyl)amino)-4-methyl-3-(trifluoromethyl)pyridin-2-yl)-8-chloro-10-fluoro-2-((3-methylene-1-azabicyclo[3.2.0]heptan-5-yl)methoxy)-5,6-dihydro-4H-[1,4]oxazepino[5,6,7-de]quinazolin-4-yl)ethyl)pyridin-2-yl)carbamate

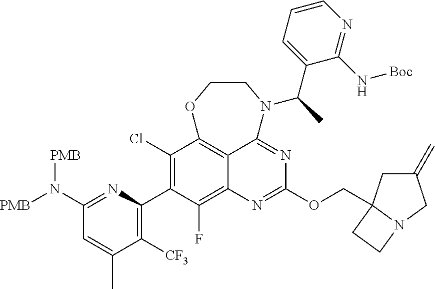

To a solution of (3-methylene-1-azabicyclo[3.2.0]heptan-5-yl)methanol (55.0 mg, 0.4 mmol) in tetrahydrofuran (3 mL) was added sodium hydride (16.0 mg, 0.67 mmol, 60% dispersion in mineral oil), the mixture was stirred at 0° C. for 10 minutes. Then tert-butyl N-[3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5,7,9(14)-pentaen-13-yl]ethyl]-2-pyridyl]-N-tert-butoxycarbonyl-carbamate (desired atropisomer) (150.0 mg, 0.13 mmol) was added and stirred at 60° C. for 1 hour. After completion, the reaction was quenched with saturated ammonium chloride solution. The resulting solution was diluted with water and extracted with ethyl acetate. The organic layers were combined, washed with brine, dried over anhydrous sodium sulfate and concentrated. The residue was purified by flash chromatography on silica gel eluting with dichloromethane/methanol (92:8) to afford the title compound (60.0 mg, 0.06 mmol, 45.4% yield) as a yellow solid. LC-MS: (ESI, m/z): 1011.4 [M+H]⁺

Step 6: (6R)-6-(4-((R)-1-(2-aminopyridin-3-yl)ethyl)-8-chloro-10-fluoro-2-((3-methylene-1-azabicyclo[3.2.0]heptan-5-yl)methoxy)-5,6-dihydro-4H-[1,4]oxazepino[5,6,7-de]quinazolin-9-yl)-4-methyl-5-(trifluoromethyl)pyridin-2-amine

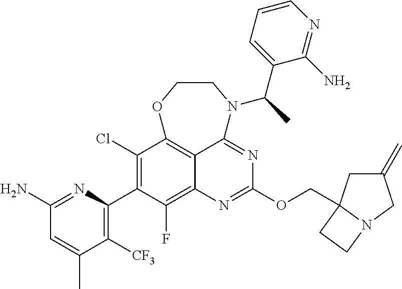

A solution of tert-butyl (3-((1R)-1-(9-((R)-6-(bis(4-methoxybenzyl)amino)-4-methyl-3-(trifluoromethyl)pyridin-2-yl)-8-chloro-10-fluoro-2-((3-methylene-1-azabicyclo[3.2.0]heptan-5-yl)methoxy)-5,6-dihydro-4H-[1,4]oxazepino[5,6,7-de]quinazolin-4-yl)ethyl)pyridin-2-yl)carbamate (50.0 mg, 0.05 mmol) in trifluoroacetic acid (1 mL) was stirred at 50° C. for 1 hour. After completion, the solvent was removed under vacuum. The resulting residue was purified by reverse phase chromatography (acetonitrile/0.1% ammonium bicarbonate in water) to afford the title compound (30.0 mg, 0.04 mmol, 84.1% yield). LC-MS: (ESI, m/z): 671.3 [M+H]⁺.

Example 118: ¹H NMR (300 MHz, DMSO-d₆, ppm) δ 7.98 (dd, J=4.9, 1.7 Hz, 1H), 7.74-7.58 (m, 1H), 6.82 (s, 2H), 6.67 (dd, J=7.5, 4.9 Hz, 1H), 6.48 (s, 1H), 6.33-6.18 (m, 1H), 5.84-5.60 (m, 2H), 5.20 (s, 1H), 5.12 (s, 1H), 4.55-4.34 (m, 3H), 4.32-4.17 (m, 1H), 3.79-3.55 (m, 2H), 3.54-3.39 (m, 2H), 3.30-3.13 (m, 1H), 3.11-2.90 (m, 1H), 2.67 (s, 2H), 2.47-2.40 (m, 1H), 2.37 (d, J=2.1 Hz, 3H), 2.13-1.97 (m, 1H), 1.58 (d, J=6.7 Hz, 3H).

Example 119: 4-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)naphthalen-2-ol

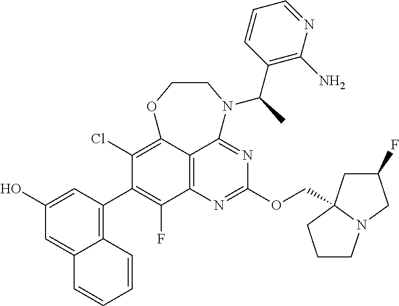 [SMILES: C[C@H](c1cccnc1N)N1CCOc2c(Cl)c(-c3cc(O)cc4ccccc34)c(F)c3nc(OC[C@@]45CCCN4C[C@H](F)C5)nc1c23]

### Synthetic Route

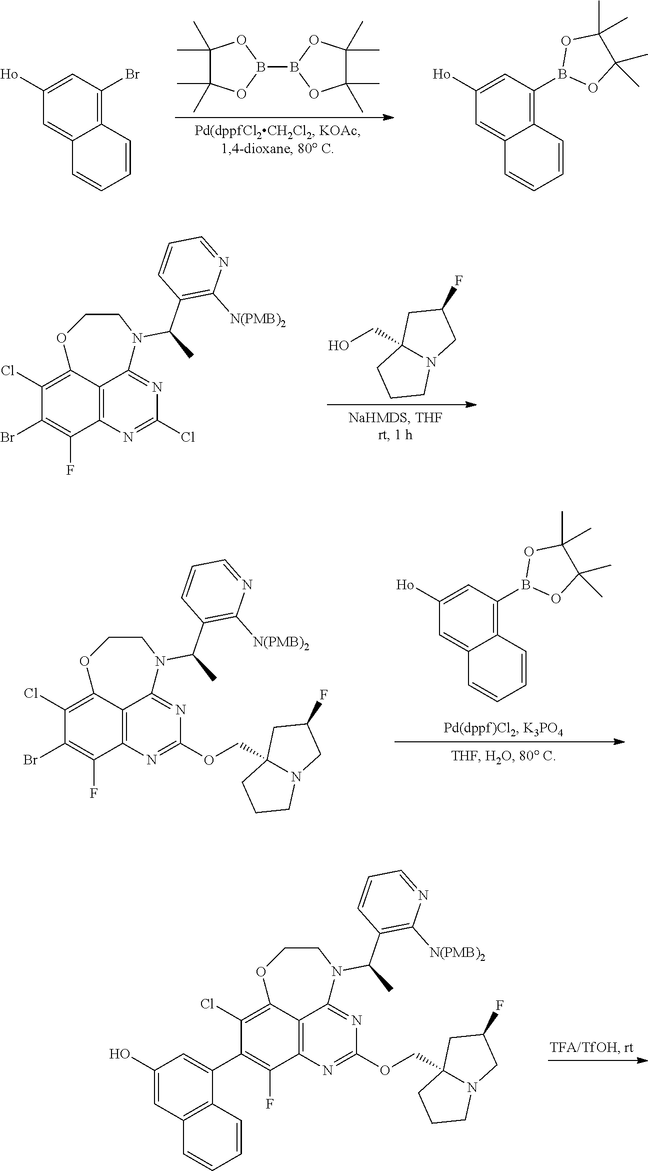

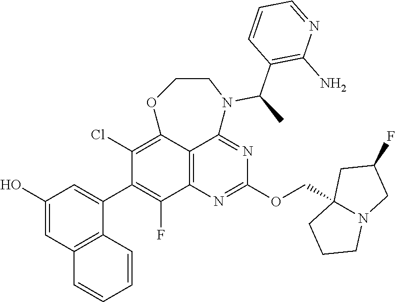 [SMILES: C[C@H](c1cccnc1N)N1CCOc2c(Cl)c(-c3cc(O)cc4ccccc34)c(F)c3nc(OC[C@@]45CCCN4C[C@H](F)C5)nc1c23]

Step 1: 4-(4,4,5,5-tetramethyl-1,3,2-dioxaborolan-2-yl)naphthalen-2-ol

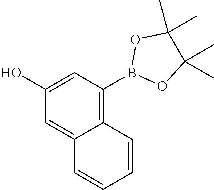 [SMILES: CC1(C)OB(c2cc(O)cc3ccccc23)OC1(C)C]

A mixture of 4-bromonaphthalen-2-ol (4.00 g, 18.0 mmol), bis(pinacolato)diboron (9.20 g, 36.0 mmol), 1,1′-bis(diphenylphosphino)ferrocene-palladium(II)dichloride dichloromethane complex (1.50 g, 1.8 mmol) and potassium acetate (5.3 g, 54.0 mmol) in 1,4-dioxane (40.0 mL) was stirred at 80° C. for 2 hours. After completion, the reaction mixture was concentrated in vacuum, diluted with water and extracted with dichloromethane. The combined organic layers were washed with brine, dried over anhydrous sodium sulfate and concentrated in vacuum. The residue was purified by flash chromatography on silica gel eluting with petroleum ether/ethyl acetate (20:1) to afford the title compound (5.0 g, 17.8 mmol, 98% yield) as a light yellow solid.

Step 2: 3-((R)-1-(9-bromo-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N,N-bis(4-methoxybenzyl)pyridin-2-amine

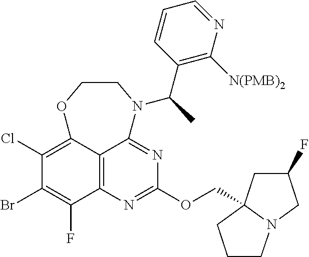

Under nitrogen, sodium bis(trimethylsilyl)amide (1.8 mL, 1.8 mmol, 1 M in tetrahydrofuran) was dropwise added to a solution of ((2R,7aS)-2-fluorotetrahydro-1H-pyrrolizin-7a(5H)-yl)methanol (224.0 mg, 1.4 mmol) in tetrahydrofuran (10 mL) at room temperature, and the mixture was stirred at room temperature for 0.5 hour. Then the mixture was added to a solution of 3-[(1R)-1-(7-bromo-3,8-dichloro-6-fluoro-10-oxa-2,4,13-triazatricyclo[7.4.1.05,14]tetradeca-1,3,5(14),6,8-pentaen-13-yl)ethyl]-N,N-bis[(4-methoxyphenyl)methyl]pyridin-2-amine (500.0 mg, 0.7 mmol) in tetrahydrofuran (10 mL) and stirred at room temperature for 1 hour. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with petroleum ether/ethyl acetate (4:1) to afford the title compound (510.0 mg, 0.61 mmol, 87% yield). LC-MS: (ESI, m/z): 835.2, [M+H]⁺

Step 3: 4-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)naphthalen-2-ol

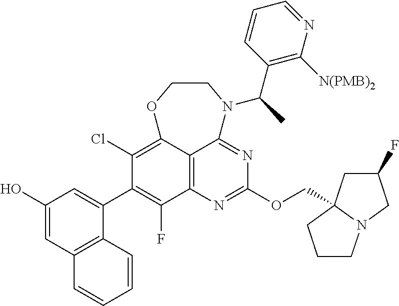

A mixture of 3-((R)-1-(9-bromo-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N,N-bis(4-methoxybenzyl)pyridin-2-amine (300.0 mg, 0.36 mmol), 4-(4,4,5,5-tetramethyl-1,3,2-dioxaborolan-2-yl)naphthalen-2-ol (146.0 mg, 0.54 mmol), [1,1′-Bis(diphenylphosphino)ferrocene]dichloropalladium(II) (26.0 mg, 0.04 mmol) and potassium phosphate (153.0 mg, 0.72 mmol) in tetrahydrofuran (8.0 mL) and water (2 ml) was stirred at 80° C. for 1 hour. After completion, the reaction mixture was diluted with water and extracted with dichloromethane. The combined organic layers were washed with brine, anhydrous sodium sulfate and concentrated in vacuum. The residue was purified by flash chromatography on silica gel eluting with dichloromethane/methanol (10:1) to afford the title compound (160.0 mg, 0.18 mmol, 49% yield) as a light yellow solid. LC-MS: (ESI, m/z): 900.0 [M+H]⁺

Step 4: 4-(4-((R)-1-(2-Aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)naphthalen-2-ol

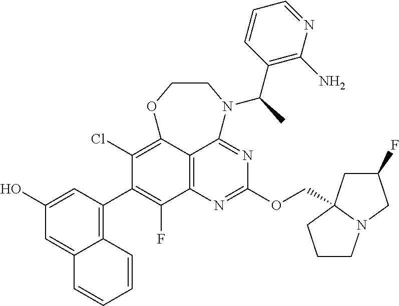 [SMILES: C[C@H](c1cccnc1N)N1CCOc2c(Cl)c(-c3cc(O)cc4ccccc34)c(F)c3nc(OC[C@@]45CCCN4C[C@H](F)C5)nc1c23]

A solution of 4-(4-((R)-1-(2-(bis(4-methoxybenzyl)amino)pyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)naphthalen-2-ol (140.0 mg, 0.16 mmol) in trifluoroacetic acid (2.0 mL) and trifluoromethanesulfonic acid (0.2 mL) was stirred at room temperature for 1 hour. After completion, the reaction mixture was diluted with dichloromethane, basified with sat. NaHCO₃ aq. to adjust pH to 10, extracted with dichloromethane, washed with brine, dried over anhydrous sodium sulfate and concentrated in vacuum. The residue was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 34% B to 56% B in 9 min, 56% B; Wave Length: 254/220 nm: RT1(min): 9.6 to afford the title compound (13.0 mg, 0.02 mmol, 12% yield). LC-MS: (ESI, m/z): 659.2[M+H]⁺

Example 119: ¹H NMR (300 MHz, Methanol-d₄, ppm) δ 7.96 (d, J=5.3 Hz, 1H), 7.81-7.69 (m, 2H), 7.43-7.35 (m, 1H), 7.30-7.12 (m, 3H), 7.00 (d, J=2.4 Hz, 1H), 6.79 (dd. J=7.4, 5.1 Hz, 1H), 6.55 (dd, J=13.8, 7.0 Hz, 1H), 5.30 (d, J=54.0 Hz, 1H), 4.53-4.44 (m, 1H), 4.43-4.17 (m, 3H), 3.80-3.70 (m, 1H), 3.59-3.50 (m, 1H), 3.28-3.13 (m, 3H), 3.05-2.96 (m, 1H), 2.27-2.09 (m, 3H), 2.04-1.87 (m, 3H), 1.69 (d, J=6.9 Hz, 3H)

Example 120a & 120b: 6-((R)-4-((R)-1-(2-aminopyridin-3-yl)ethyl)-8-chloro-10-fluoro-2-(((7S,9aS)-hexahydro-1H,3H-pyrrolo[2,1-c][1,4]oxazepin-7-yl)methoxy)-5,6-dihydro-4H-[1,4]oxazepino[5,6,7-de]quinazolin-9-yl)-4-methyl-5-(trifluoromethyl)pyridin-2-amine and 6-((R)-4-((R)-1-(2-aminopyridin-3-yl)ethyl)-8-chloro-10-fluoro-2-(((7R,9aR)-hexahydro-1H,3H-pyrrolo[2,1-c][1,4]oxazepin-7-yl)methoxy)-5,6-dihydro-4H-[1,4]oxazepino[5,6,7-de]quinazolin-9-yl)-4-methyl-5-(trifluoromethyl)pyridin-2-amine

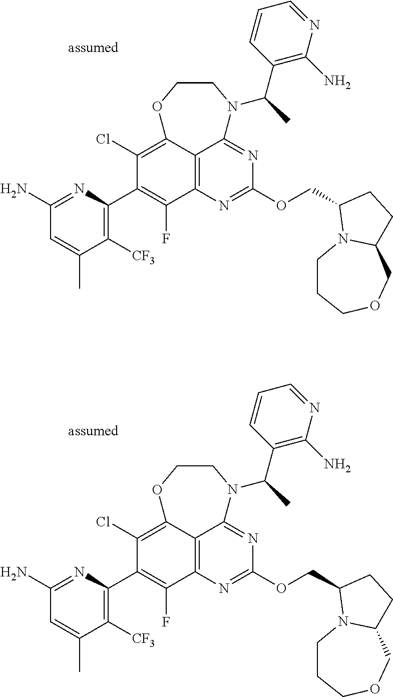

### Synthetic Route:

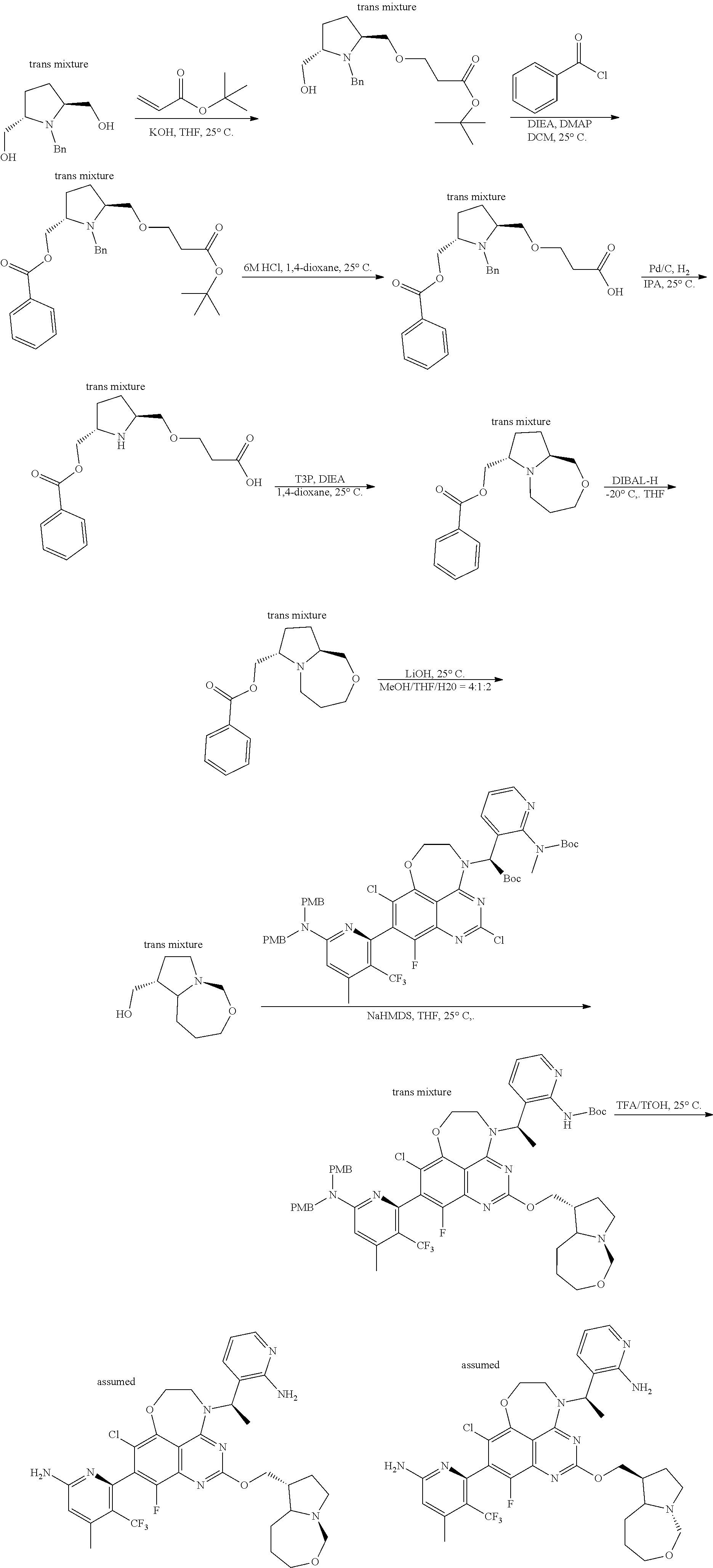

Step 1: tert-butyl 3-(((2S,5S)-1-benzyl-5-(hydroxymethyl)pyrrolidin-2-yl)methoxy)propanoate

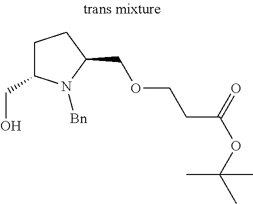 [SMILES: CC(C)(C)OC(=O)CCOC[C@@H]1CC[C@@H](CO)N1Cc1ccccc1]

A solution of ((2S,5S)-1-benzylpyrrolidine-2,5-diyl)dimethanol (5.00 g, 22.59 mmol, trans mixture) in tetrahydrofuran (50 mL) was added potassium hydroxide (250.0 mg, 4.46 mmol) and stirred for 20 minutes. Then tert-butyl acrylate (3.14 mL, 21.46 mmol) was added and stirred at 25° C. for 1 hour. After completion, the solvent was concentrated under vacuum. The residue was purified by reverse chromatography on C18 gel eluting with water (0.1% ammonium bicarbonate)(A)/acetonitrile(B) (5-95% in 30 min) to afford the title compound (2.20 g, 6.29 mmol, 27.9% yield, trans mixture) as a yellow oil. LC-MS: (ESI, m/z): 350.5 [M+H]⁺

Step 2: ((2S,5S)-1-benzyl-5-((3-(tert-butoxy)-3-oxopropoxy)methyl)pyrrolidin-2-yl)methyl benzoate (Trans Mixture)

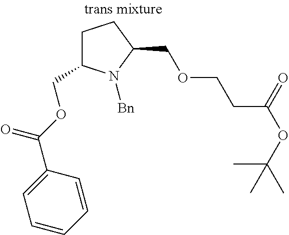 [SMILES: CC(C)(C)OC(=O)CCOC[C@@H]1CC[C@@H](COC(=O)c2ccccc2)N1Cc1ccccc1]

A solution of tert-butyl 3-(((2S,5S)-1-benzyl-5-(hydroxymethyl)pyrrolidin-2-yl)methoxy)propanoate (1.80 g, 5.15 mmol, trans mixture), N,N-diisopropylethylamine (2.0 g, 15.5 mmol) and 4-(dimethylamino)pyridine (125.0 mg, 1.02 mmol) in dichloromethane (3 mL) was added benzoyl chloride (1.2 mL, 10.32 mmol) and stirred at 25° C. for 1 hour. After completion, the solvent was concentrated under vacuum. The residue was purified by flash chromatography on silica gel eluting with petroleum ether/ethyl acetate (1:1) to afford the title compound (1.50 g, 3.31 mmol, 64.2% yield, trans mixture) as a yellow oil. LC-MS: (ESI, m/z): 454.7 [M+H]⁺

Step 3: 3-(((2S,5S)-5-((benzoyloxy)methyl)-1-benzylpyrrolidin-2-yl)methoxy)propanoic acid (Trans Mixture)

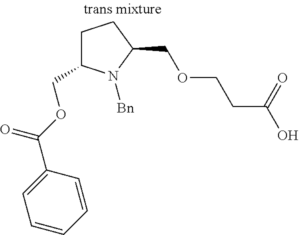 [SMILES: O=C(O)CCOC[C@@H]1CC[C@@H](COC(=O)c2ccccc2)N1Cc1ccccc1]

A solution of ((2S,5S)-1-benzyl-5-((3-(tert-butoxy)-3-oxopropoxy)methyl)pyrrolidin-2-yl)methyl benzoate (650.0 mg, 1.43 mmol, trans mixture) and hydrochloric acid (6.45 mL, 38.69 mmol, 6M) in 1,4-dioxane (8 mL) was stirred at 25° C. for 2 hours. After completion, the solvent was concentrated under vacuum. LC-MS showed the product formed. The crude product was used in the next step without purification. LC-MS: (ESI, m/z): 398.2 [M+H]⁺

Step 4: 3-(((2S,5S)-5-((benzoyloxy)methyl)pyrrolidin-2-yl)methoxy)propanoic acid (Trans Mixture)

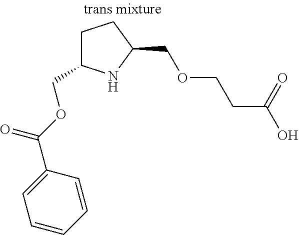 [SMILES: O=C(O)CCOC[C@@H]1CC[C@@H](COC(=O)c2ccccc2)N1]

A solution of 3-(((2S,5S)-5-((benzoyloxy)methyl)-1-benzylpyrrolidin-2-yl)methoxy)propanoic acid (700.0 mg, 1.76 mmol, trans mixture) and Pd on charcoal (0.7 g, ca. 10% Pd) in methyl alcohol (10 mL) was stirred at 25° C. for 1 hour. After completion, the reaction solution was filtered. The filtrate was concentrated under reduced pressure to afford the crude product which was directly used in the next step without purification. LC-MS: (ESI, m/z): 308.1 [M+H]⁺

Step 5: ((7S,9aS)-5-oxohexahydro-1H,3H-pyrrolo[2,1-c][1,4]oxazepin-7-yl)methyl benzoate (Trans Mixture)

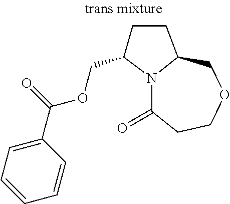 [SMILES: O=C(OC[C@@H]1CC[C@H]2COCCC(=O)N21)c1ccccc1]

A solution of 3-(((2S,5S)-5-((benzoyloxy)methyl)pyrrolidin-2-yl)methoxy)propanoic acid (700.0 mg, 2.28 mmol, trans mixture), propylphosphonic anhydride (2.17 g, 6.82 mmol) and N,N-diisopropylethylamine (980.0 mg, 7.6 mmol) in 1,4-dioxane (2 mL) was stirred at 25° C. for 1 hour. After completion, the reaction mixture was diluted with water. The resulting solution was extracted with ethyl acetate and washed with brine. The organic layer was dried over anhydrous sodium sulfate and concentrated under vacuum. The residue was purified by flash chromatography on silica gel eluting with dichloromethane/methanol (9:1) to afford the title compound (430.0 mg, 1.49 mmol, 65.3% yield, trans mixture) as a yellow solid. LC-MS: (ESI, m/z): 290.1 [M+H]⁺

Step 6: ((7S,9aS)-hexahydro-1H,3H-pyrrolo[2,1-c][1,4]oxazepin-7-yl)methyl benzoate (trans mixture)

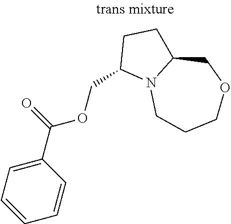 [SMILES: O=C(OC[C@@H]1CC[C@H]2COCCCN21)c1ccccc1]

A solution of ((7S,9aS)-5-oxohexahydro-1H,3H-pyrrolo[2,1-c][1,4]oxazepin-7-yl)methyl benzoate (350.0 mg, 1.21 mmol, trans mixture) in tetrahydrofuran (4 mL) was added and diisobutylaluminum hydride (3.63 mL, 3.63 mmol, 1M in toluene) and stirred at −20° C. for 2 hours. After completion, the reaction was quenched with sodium sulfate decahydrate and diluted with tetrahydrofuran. The resulted solution was filtrated and filtrate was concentrated under reduced pressure. The residue was purified by reverse chromatography on C18 gel eluting with water (0.1% ammonium bicarbonate)/acetonitrile (5-95% in 30 min) to afford the title compound (70.0 mg, 0.25 mmol, 21% yield, trans mixture) as a yellow solid. LC-MS: (ESI, m/z): 276.2 [M+H]⁺

Step 7: ((7S,9aS)-hexahydro-1H,3H-pyrrolo[2,1-c][1,4]oxazepin-7-yl)methanol (Trans Mixture)

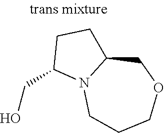 [SMILES: OC[C@@H]1CC[C@H]2COCCCN12]

A solution of ((7S,9aS)-hexahydro-1H,3H-pyrrolo[2,1-c][1,4]oxazepin-7-yl)methyl benzoate (70.0 mg, 0.25 mmol, trans mixture) and lithium hydroxide (21.0 mg, 0.88 mmol) in tetrahydrofuran (2 mL), water (0.8 mL) and methyl alcohol (0.4 mL) was stirred at 25° C. for 2 hours. After completion, the reaction mixture was adjusted PH=7.0 with HCl/dioxane and concentrated under vacuum. Then the residue was diluted with dichloromethane, filtrated and filtrate was concentrated under reduced pressure. The crude product was used in the next step without purification. LC-MS: (ESI, m/z): 172.2 [M+H]⁺

Step 8: tert-butyl (3-((R)-1-((R)-9-(6-(bis(4-methoxybenzyl)amino)-4-methyl-3-(trifluoromethyl)pyridin-2-yl)-8-chloro-10-fluoro-2-(((7S,9aS)-hexahydro-1H,3H-pyrrolo[2,1-c][1,4]oxazepin-7-yl)methoxy)-5,6-dihydro-4H-[1,4]oxazepino[5,6,7-de]quinazolin-4-yl)ethyl)pyridin-2-yl)carbamate (trans mixture)

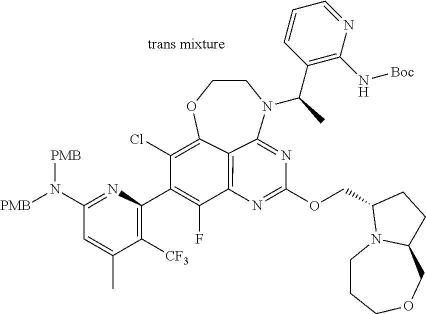

Under nitrogen, a solution of ((7S,9aS)-hexahydro-1H,3H-pyrrolo[2,1-c][1,4]oxazepin-7-yl)methanol (40.0 mg, 0.23 mmol, trans mixture) in tetrahydrofuran (0.5 mL) was added sodium bis(trimethylsilyl)amide (0.3 mL, 0.30 mmol, 1M tetrahydrofuran) at 0° C. for 30 minutes. Then tert-butyl N-tert-butoxycarbonyl-N-(3-(rac-(1R)-1-(7-(6-(bis((4-methoxyphenyl)methyl)amino)-4-methyl-3-(trifluoromethyl)-2-pyridyl)-3,8-dichloro-6-fluoro-10-oxa-2,4,13-triazatricyclo[7.4.1.05,14]tetradeca-1,3,5,7,9(14)-pentaen-13-yl)ethyl)-2-pyridyl)carbamate (desired atropisomer) (100.0 mg, 0.10 mmol) was added and stirred at 25° C. for 1 hour. After completion, the reaction mixture was diluted with water. The resulting solution was extracted with ethyl acetate and washed with brine. The organic layer was dried over anhydrous sodium sulfate and concentrated under vacuum. The residue was purified by flash chromatography on silica gel eluting with dichloromethane/methanol to afford the title compound (40.0 mg, 0.038 mmol, 38.7% yield, trans mixture) as a yellow solid. LC-MS: (ESI, m/z): 1043.5 [M+H]⁺

Step 9: 6-((R)-4-((R)-1-(2-aminopyridin-3-yl)ethyl)-8-chloro-10-fluoro-2-(((7S,9aS)-hexahydro-1H,3H-pyrrolo[2,1-c][1,4]oxazepin-7-yl)methoxy)-5,6-dihydro-4H-[1,4]oxazepino[5,6,7-de]quinazolin-9-yl)-4-methyl-5-(trifluoromethyl)pyridin-2-amine and 6-((R)-4-((R)-1-(2-aminopyridin-3-yl)ethyl)-8-chloro-10-fluoro-2-(((7R,9aR)-hexahydro-1H,3H-pyrrolo[2,1-c][1,4]oxazepin-7-yl)methoxy)-5,6-dihydro-4H-[1,4]oxazepino[5,6,7-de]quinazolin-9-yl)-4-methyl-5-(trifluoromethyl)pyridin-2-amine

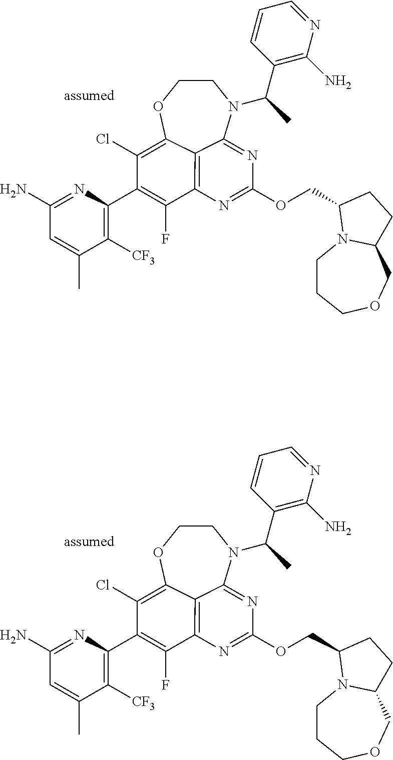

A solution of tert-butyl (3-((R)-1-((R)-9-(6-(bis(4-methoxybenzyl)amino)-4-methyl-3-(trifluoromethyl)pyridin-2-yl)-8-chloro-10-fluoro-2-(((7S,9aS)-hexahydro-1H,3H-pyrrolo[2,1-c][1,4]oxazepin-7-yl)methoxy)-5,6-dihydro-4H-[1,4]oxazepino[5,6,7-de]quinazolin-4-yl)ethyl)pyridin-2-yl)carbamate (50.0 mg, 0.050 mmol) in trifluoroacetic acid (1.0 mL) and trifluoromethanesulfonic acid (10.1 mL) was stirred at 25° C. for 30 minutes. After completion, the solvent was concentrated under vacuum. The residue was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 30% B to 55% B in 9 min, 55% B; Wave Length: 254/220 nm: RT1(min): 8.9; Number Of Runs: 0 and Chiral-Prep-HPLC with the following conditions: Column: CHIRALPAK IE-3, 4.6*50 mm, 3 μm; Mobile Phase A: Hex:DCM=3:1) (0.5% IPAmine):IPA=60:40; Flow rate: 1 mL/min: Gradient: 0% B to 0% B; Injection Volume: 5 ul mL to afford 6-((R)-4-((R)-1-(2-aminopyridin-3-yl)ethyl)-8-chloro-10-fluoro-2-(((7S,9aS)-hexahydro-1H,3H-pyrrolo[2,1-c][1,4]oxazepin-7-yl)methoxy)-5,6-dihydro-4H-[1,4]oxazepino[5,6,7-de]quinazolin-9-yl)-4-methyl-5-(trifluoromethyl)pyridin-2-amine (2.5 mg, 0.0036 mmol, 7.4% yield) and 6-((R)-4-((R)-1-(2-aminopyridin-3-yl)ethyl)-8-chloro-10-fluoro-2-(((7R,9aR)-hexahydro-1H,3H-pyrrolo[2,1-c][1,4]oxazepin-7-yl)methoxy)-5,6-dihydro-4H-[1,4]oxazepino[5,6,7-de]quinazolin-9-yl)-4-methyl-5-(trifluoromethyl)pyridin-2-amine (1.3 mg, 0.0018 mmol, 3.9% yield).

Example 120a: ¹H NMR (300 MHz, Methanol-d₄, ppm) δ 7.95 (dd, J=5.1, 1.7 Hz, 1H), 7.75 (d, J=7.5 Hz, 1H), 6.77 (dd, J=7.5, 5.1 Hz, 1H), 6.61-6.56 (m, 1H), 6.52 (q, J=6.8 Hz, 1H), 4.58 (dd, J=11.1, 5.5 Hz, 2H), 4.43 (dd, J=11.5, 5.6 Hz, 2H), 4.27 (dd, J=12.4, 6.8 Hz, 1H), 3.85-3.76 (m, 1H), 3.73-3.55 (m, 5H), 3.55-3.38 (m, 2H), 3.27-3.16 (m, 1H), 2.49-2.33 (m, 3H), 2.21-2.07 (m, 1H), 2.04-1.90 (m, 2H), 1.85-1.68 (m, 2H), 1.66 (d, J=6.9 Hz, 3H), 1.59-1.49 (d, J=10.6 Hz, 1H). LC-MS: (ESI, m/z): 703.1 [M+H]⁺

Example 120b: ¹H NMR (300 MHz, Methanol-d₄, ppm) δ 7.96 (dd, J=5.1, 1.7 Hz, 1H), 7.76 (d, J=7.5 Hz, 1H), 6.78 (dd, J=7.5, 5.1 Hz, 1H), 6.59 (s, 1H), 6.51 (q, J=6.9 Hz, 1H), 4.64-4.37 (m, 4H), 4.28 (dd, J=12.2, 6.3 Hz, 1H), 3.85-3.74 (m, 1H), 3.73-3.58 (m, 5H), 3.56-3.45 (m, 2H), 2.44 (s, 3H), 2.21-2.09 (m, 1H), 2.06-1.91 (m, 2H), 1.87-1.70 (m, 2H), 1.69-1.54 (d, J=6.9 Hz, 5H). LC-MS: (ESI, m/z): 703.1 [M+H]⁺

Example 121: (S)-5-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)pyrrolidin-2-one

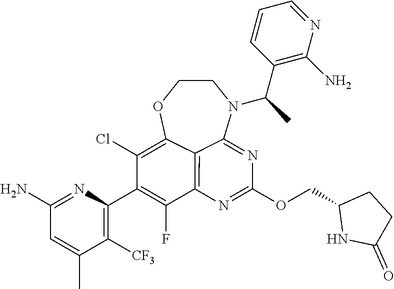

### Synthetic Route:

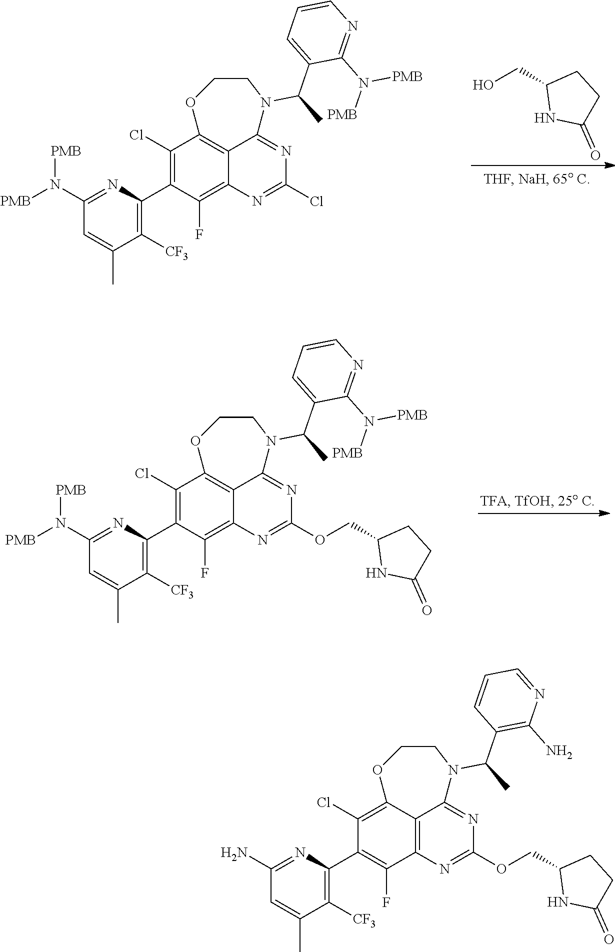

Step 1: (S)-5-(((9-((R)-6-(bis(4-methoxybenzyl)amino)-4-methyl-3-(trifluoromethyl)pyridin-2-yl)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-2-yl)oxy)methyl)pyrrolidin-2-one

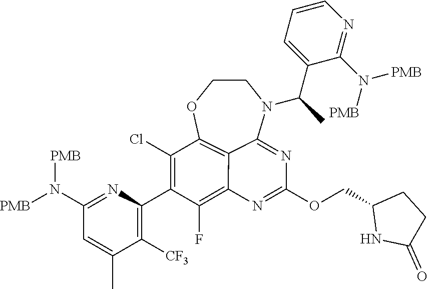

Similar to as described in General Procedure A. A solution of (5S)-5-(hydroxymethyl)-2-pyrrolidinone (88.0 mg, 0.7600 mmol) in tetrahydrofuran (4 mL) was added sodium hydride (76.0 mg, 1.9 mmol, 60% dispersion in mineral oil) was stirred at 0° C. for 5 minutes. Then (R)-6-(4-((R)-1-(2-(bis(4-methoxybenzyl)amino)pyridin-3-yl)ethyl)-2,8-dichloro-10-fluoro-5,6-dihydro-4H-[1, 4]oxazepino[5,6,7-de]quinazolin-9-yl)-N,N-bis(4-methoxybenzyl)-4-methyl-5-(trifluoromethyl)pyridin-2-amine (400.0 mg, 0.38 mmol) was added and stirred at 65° C. for 2 hours. After completion, the reaction was quenched with ammonium chloride solution. The reaction mixture was diluted with water.

The resulting solution was extracted with ethyl acetate and washed with brine. The organic layer was dried over anhydrous sodium sulfate and concentrated under vacuum. The residue was purified by flash chromatography on silica gel eluting with dichloromethane/methanol (10:1) to afford the title compound (150 mg, 0.1330 mmol, 34.8% yield) as a yellow solid. LC-MS: (ESI, m/z): 1127.6 [M+H]⁺

Step 2: (S)-5-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)pyrrolidin-2-one

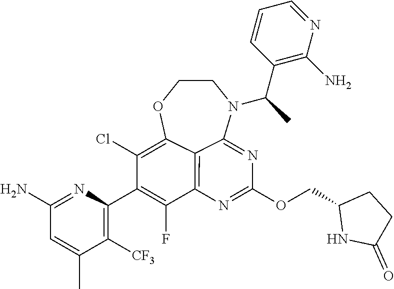

A solution of (S)-5-(((9-((R)-6-(bis(4-methoxybenzyl)amino)-4-methyl-3-(trifluoromethyl)pyridin-2-yl)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-2-yl)oxy)methyl)pyrrolidin-2-one (100.0 mg, 0.09 mmol) in trifluoroacetic acid (2 mL) and trifluoromethanesulfonic acid (0.2 mL) was stirred at 25° C. for 0.5 hour. After completion, the solvent was concentrated under vacuum. The residue was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 17% B to 42% B in 9 min, 42% B; Wave Length: 254/220 nm; RT1(min): 8.9 to afford the title compound (35.6 mg, 0.055 mmol, 62% yield). LC-MS: (ESI, m/z): 647.0 [M+H]⁺

Example 121: ¹H NMR (300 MHz, DMSO-d₆, ppm) δ 7.98 (s, 1H), 7.86 (s, 1H), 7.64 (d, J=8.5 Hz, 1H), 6.82 (s, 2H), 6.74-6.62 (m, 1H), 6.48 (s, 1H), 6.24 (q, J=6.2 Hz, 1H), 5.69 (s, 2H), 4.50-4.36 (m, 1H), 4.36-4.18 (m, 3H), 3.94 (s, 1H), 3.66 (dd. J=15.9, 6.8 Hz, 1H), 3.45-3.34 (m, 1H), 2.36 (s, 3H), 2.32-2.02 (m, 3H), 1.97-1.81 (m, 1H), 1.57 (d, J=6.1 Hz, 3H).

Example 122a & 122b: 6-((R)-4-((R)-1-(2-aminopyridin-3-yl)ethyl)-8-chloro-2-(((S)-4,4-difluoro-1,2-dimethylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-2-(((R)-4,4-difluoro-1,2-dimethylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

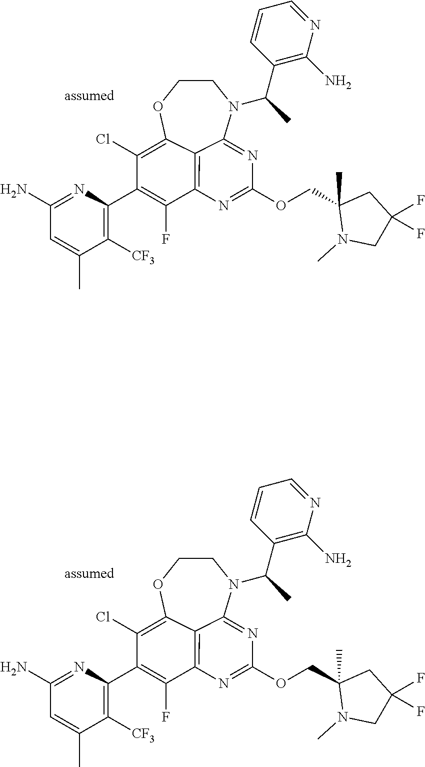

### Synthetic Route

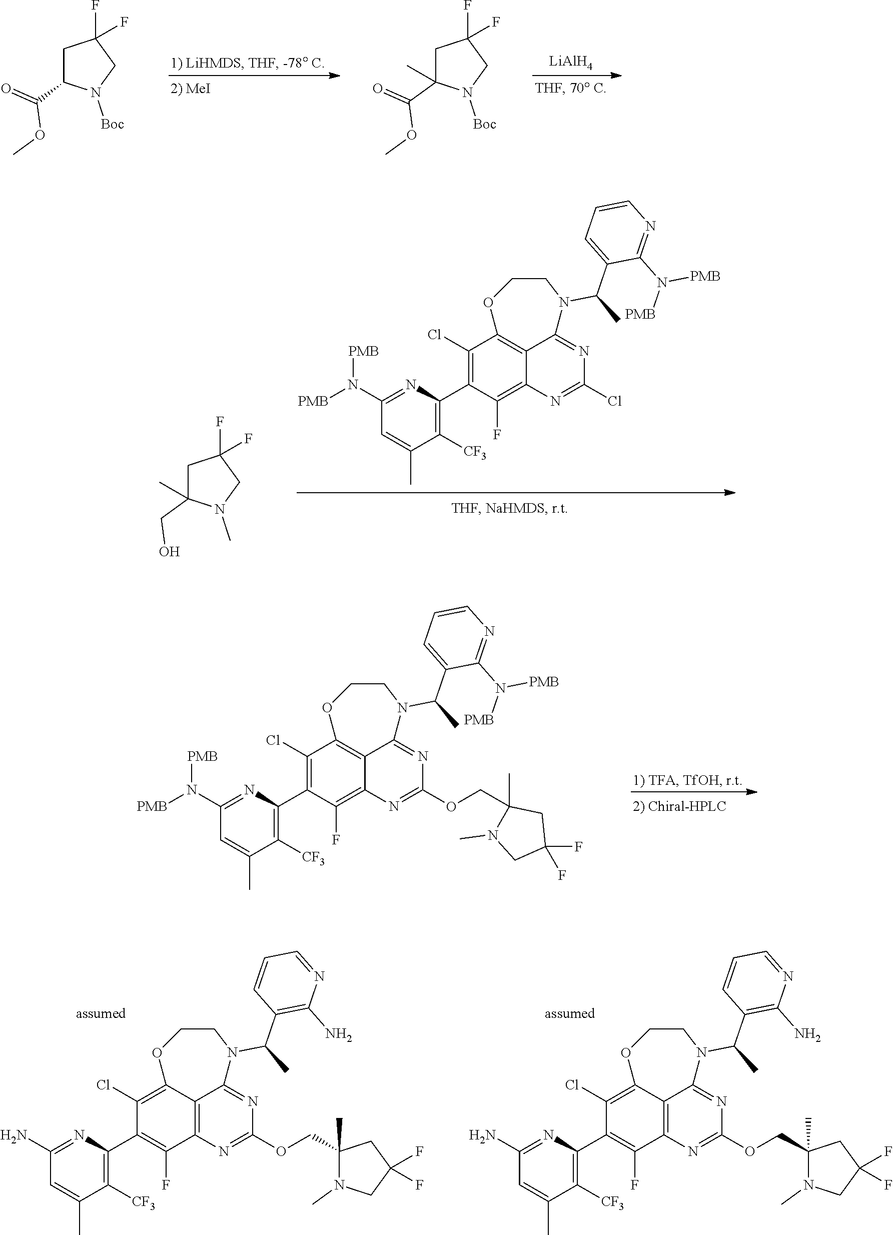

Step 1: 1-(tert-butyl) 2-methyl 4,4-difluoro-2-methylpyrrolidine-1,2-dicarboxylate

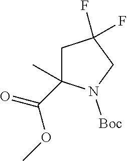 [SMILES: COC(=O)C1(C)CC(F)(F)CN1C(=O)OC(C)(C)C]

Under nitrogen, a solution of 1-tert-butyl 2-methyl (2S)-4,4-difluoropyrrolidine-1,2-dicarboxylate (5.00 g, 18.85 mmol) was dropwise added lithium bis(trimethylsilyl)amide (28.28 mL, 28.28 mmol, 1M in tetrahydrofuran) in tetrahydrofuran (50 mL) and stirred at −78° C. for 30 mins. Then Iodomethane (8.05 g, 56.71 mmol) was dropwise added and stirred at −78° C. for 1 hour. After completion, the reaction was quenched with saturated ammonium chloride solution. The reaction was diluted with ethyl acetate, washed with saturated brine, dried over anhydrous sodium sulfate and concentrated under vacuum to afford 1-(tert-butyl) 2-methyl 4,4-difluoro-2-methylpyrrolidine-1,2-dicarboxylate (5.00 g, 17.90 mmol, 95% yield) as a yellow oil. LC-MS: (ESI, m/z): 280.1 [M+H]⁺

Step 2: (4,4-difluoro-1,2-dimethyl-pyrrolidin-2-yl)methanol

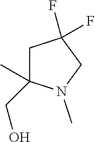

A solution of 1-(tert-butyl) 2-methyl 4,4-difluoro-2-methylpyrrolidine-1,2-dicarboxylate (1.00 g, 3.58 mmol) in tetrahydrofuran (10 mL) was added lithium aluminum hydride (250.0 mg, 6.58 mmol) at 0° C. and stirred at 70° C. for 2 hours. After completion, the reaction was cooled to room temperature, quenched with sodium sulfate decahydrate and filtered. After filtration, the filtrate was concentrated under reduced pressure to afford (4,4-difluoro-1,2-dimethyl-pyrrolidin-2-yl)methanol (400.0 mg, 2.4 mmol, 67.6% yield) as a yellow oil. LC-MS: (ESI, m/z): 165.2 [M+H]⁺

Step 3: 6-(4-((R)-1-(2-(bis(4-methoxybenzyl)amino)pyridin-3-yl)ethyl)-8-chloro-2-((4,4-difluoro-1,2-dimethylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

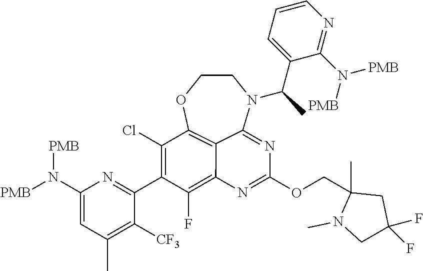

Under nitrogen, a solution of (4,4-difluoro-1,2-dimethyl-pyrrolidin-2-yl)methanol (150.0 mg, 0.91 mmol) in tetrahydrofuran (3 mL) was added sodium bis(trimethylsilyl)amide (0.95 mL, 0.95 mmol, 1M in tetrahydrofuran) at 25° C. and stirred for 20 min at 25° C. Then the reaction solution was transferred into a solution of 6-[13-[(1R)-1-[2-[bis[(4-methoxyphenyl)methyl]amino]-3-pyridyl]ethyl]-3,8-dichloro-6-fluoro-10-oxa-2,4,13-triazatricyclo[7.4.1.05,14]tetradeca-1,3,5,7,9(14)-pentaen-7-yl]-N,N-bis[(4-methoxyphenyl)methyl]-4-methyl-5-(trifluoromethyl)pyridin-2-amine (500.0 mg, 0.48 mmol) in tetrahydrofuran (5.0 mL) and stirred at 25° C. for 2 hours. After completion, the reaction was quenched with saturated ammonium chloride solution, diluted with ethyl acetate, washed with saturated brine, dried over anhydrous sodium sulfate and concentrated under vacuum. The residue was purified by flash chromatography on silica gel eluting with petroleum ether/ethyl acetate (3:1) to afford 6-(4-((R)-1-(2-(bis(4-methoxybenzyl)amino)pyridin-3-yl)ethyl)-8-chloro-2-((4,4-difluoro-1,2-dimethylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (500.0 mg, 0.42 mmol, 89.1% yield) as a yellow solid. LC-MS: (ESI, m/z): 1177.4 [M+H]⁺

Step 4: 6-((R)-4-((R)-1-(2-aminopyridin-3-yl)ethyl)-8-chloro-2-(((S)-4,4-difluoro-1,2-dimethylpyrrolidin-2-yl)methoxy)-10-fluoro-5,6-dihydro-4H-[1, 4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-2-(((R)-4,4-difluoro-1,2-dimethylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine

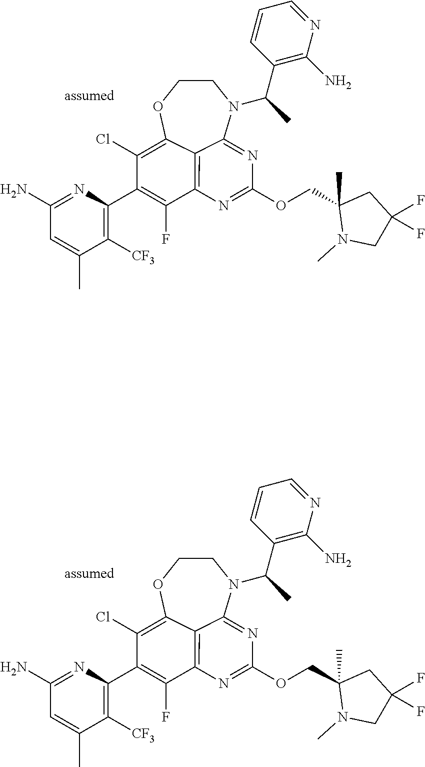

A solution of 6-(4-((R)-1-(2-(bis(4-methoxybenzyl)amino)pyridin-3-yl)ethyl)-8-chloro-2-((4,4-difluoro-1,2-dimethylpyrrolidin-2-yl)methoxy)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (500.0 mg, 0.4200 mmol) in trifluoromethanesulfonic acid (0.5 mL) and 2,2,2-trifluoroacetic acid (5 mL) was stirred at 25° C. for 10 min. After completion, the product was purified by Prep-HPLC with the following conditions (Column: Xselect CSH C18 OBD Column 30*150 mm 5 μm, n; Mobile Phase A: Water (0.1% FA), Mobile Phase B: ACN; Flow rate: 60 mL/min; Gradient: 10% B to 33% B in 8 min, 33% B; Wave Length: 254/220 nm) and Chiral-Prep-HPLC with the following conditions (Column: CHIRALPAK IF, 2*25 cm, 5 μm; Mobile Phase A: Hex:DCM=3:1(0.5% 2M NH3-MeOH)-HPLC, Mobile Phase B: EtOH-HPLC; Flow rate: 20 mL/min; Gradient: 25% B to 25% B in 9 min; Wave Length: 220/254 nm) to afford 6-((R)-4-((R)-1-(2-aminopyridin-3-yl)ethyl)-8-chloro-2-(((S)-4,4-difluoro-1,2-dimethylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine (54.7 mg, 0.078 mmol, 18.5% yield), 6-((R)-4-((R)-1-(2-aminopyridin-3-yl)ethyl)-8-chloro-2-(((R)-4,4-difluoro-1,2-dimethylpyrrolidin-2-yl)methoxy)-10-fluoro-5,6-dihydro-4H-[1,4]oxazepino[5,6,7-de]quinazolin-9-yl)-4-methyl-5-(trifluoromethyl)pyridin-2-amine (53.7 mg, 0.077 mmol, 18.1% yield).

Example 122a: ¹H NMR (300 MHz, DMSO-d₆, ppm) δ 7.97 (dd, J=5.2, 1.6 Hz, 1H), 7.71 (dd, J=7.5, 1.8 Hz, 1H), 6.81 (s, 2H), 6.72 (dd, J=7.5, 5.2 Hz, 1H), 6.47 (s, 1H), 6.32-5.95 (m, 3H), 4.47 (dd, J=12.1, 6.2 Hz, 1H), 4.39-4.20 (m, 3H), 3.71 (dd, J=15.8, 6.5 Hz, 1H), 3.54-3.39 (m, 1H), 3.39-3.33 (m, 1H), 3.06 (dd, J=14.7, 11.2 Hz, 1H), 2.63-2.51 (m, 1H), 2.36 (d, J=2.2 Hz, 3H), 2.28 (s, 3H), 2.24-2.06 (m, 1H), 1.58 (d, J=6.8 Hz, 3H), 1.14 (s, 3H). LC-MS: (ESI, m/z): 697.15 [M+H]⁺; Chiral HPLC: Column: CHIRALPAK IF-3 4.6*50 mm, 3 um; Mobile Phase: (Hex:DCM=3:1)(0.1% DEA):EtOH=80:20; Flow: 1.0 mL/min; Retention time: 1.249 min (First peak).

Example 122b: ¹H NMR (300 MHz, DMSO-d₆, ppm) δ 7.97 (dd, J=5.0, 1.7 Hz, 1H), 7.65 (dd, J=7.5, 1.8 Hz, 1H), 6.80 (s, 2H), 6.68 (dd, J=7.5, 5.0 Hz, 1H), 6.47 (s, 1H), 6.22 (q, J=6.8 Hz, 1H), 5.88 (s, 2H), 4.45 (dd, J=11.8, 6.1 Hz, 1H), 4.40-4.07 (m, 3H), 3.68 (dd, J=15.6, 6.5 Hz, 1H), 3.53-3.36 (m, 1H), 3.31-3.16 (m, 1H), 3.15-2.92 (m, 1H), 2.50-2.40 (m, 1H), 2.36 (d, J=2.2 Hz, 3H), 2.29 (s, 3H), 2.26-2.04 (m, 1H), 1.57 (d, J=6.8 Hz, 3H), 1.14 (s, 3H). LC-MS: (ESI, m/z): 697.15 [M+H]⁺ Chiral HPLC: Column: CHIRALPAK IF-3 4.6*50 mm, 3 um; Mobile Phase: (Hex:DCM=3:1)(0.1% DEA):EtOH=80:20; Flow: 1.0 mL/min; Retention time: 1.739 min (Second peak).

Example 123a & 123b: 1-((6S,8aS)-6-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one & 1-((6R,8aR)-6-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one

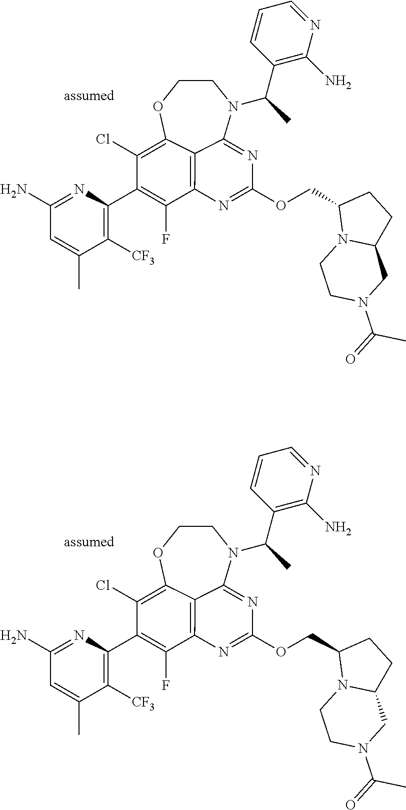

### Synthetic Route

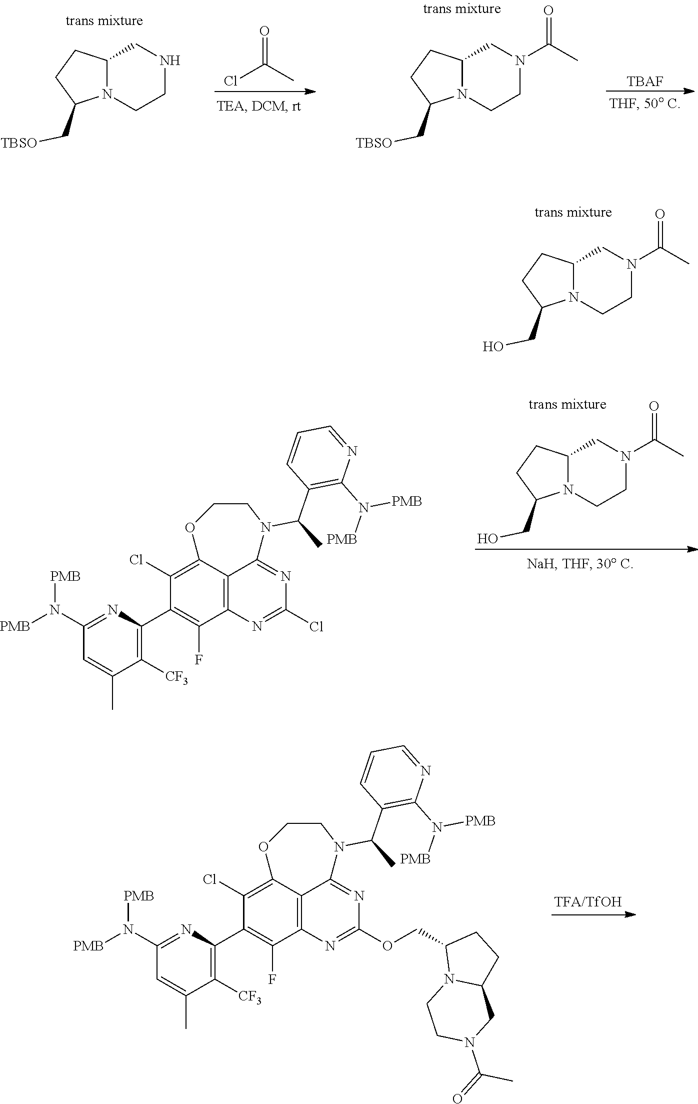

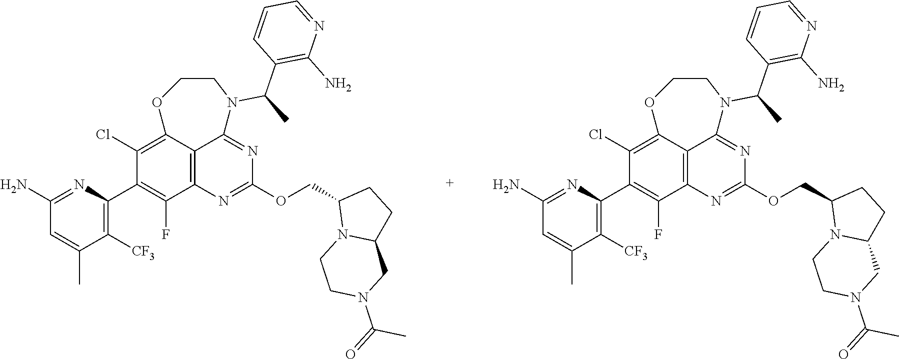

Step 1: 1-((6R,8aR)-6-(((tert-butyldimethylsilyl)oxy)methyl)hexahydropyrrolo[1,2-a]pyrazin-2(1H)-yl)ethan-1-one

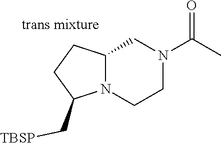

A solution of (6R,8aR)-6-(((tert-butyldimethylsilyl)oxy)methyl)octahydropyrrolo[1,2-a]pyrazine (900.0 mg, 2.0 mmol) and triethylamine (808.1 mg, 7.9 mmol) in dichloromethane (10 mL) was added acetyl chloride (156.7 mg, 2.0 mmol) and stirred at 25° C. for 2 hours. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with methanol/dichloromethane (1:30) to afford the title compound (900.0 mg, 2.0 mmol, 87.4% yield) as a white oil. LC-MS: (ESI, m/z): 313.1 [M+H]⁺.

Step 2: 1-((6R,8aR)-6-(hydroxymethyl)hexahydropyrrolo[1,2-a]pyrazin-2(1H)-yl)ethan-1-one

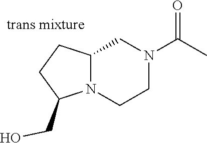

A solution of 1-((6R,8aR)-6-(((tert-butyldimethylsilyl)oxy)methyl)hexahydropyrrolo[1,2-a]pyrazin-2(1H)-yl)ethan-1-one (400.0 mg, 1.2 mmol) and tetrabutylammonium fluoride (624.0 mg, 1.5 mmol) in tetrahydrofuran (4 mL) was stirred at 50° C. for 2 hours. After completion, the residue was purified by flash chromatography on reversed-phase C18 column acetonitrile/water (0/100) to afford 1-((6R,8aR)-6-(hydroxymethyl)hexahydropyrrolo[1,2-a]pyrazin-2(1H)-yl)ethan-1-one (230.0 mg, 1.2 mmol, 90.6% yield) as a white oil. LC-MS: (ESI, m/z): 199.0 [M+H]⁺.

Step 3: 1-((6S,8aS)-6-((((R)-9-(6-(bis(4-methoxybenzyl)amino)-4-methyl-3-(trifluoromethyl)pyridin-2-yl)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one

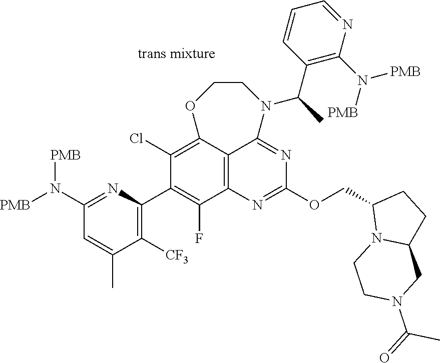

A solution of 1-[(6R,8aR)-6-(hydroxymethyl)-3,4,6,7,8,8a-hexahydro-1H-pyrrolo[1,2-a]pyrazin-2-yl]ethanone (204.1 mg, 1.0 mmol) in tetrahydrofuran (6 mL) was added sodium hydride (137.2 mg, 3.4 mmol, 60% purity) and stirred at 0° C. for 15 minutes. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (900.0 mg, 0.8 mmol) was added and stirred at 30° C. for 24 hours. After completion, the reaction was quenched with saturated ammonium chloride solution. The reaction mixture was diluted with ethyl acetate, washed with water, dried over anhydrous sodium sulfate and concentrated under vacuum. The residue was purified by flash chromatography on silica gel eluting with dichloromethane/methanol (10:1) to afford the title compound (1.00 g, 0.8 mmol, 96.3% yield) as a white solid. LC-MS: (ESI, m/z): 1210.5 [M+H]⁺.

Step 4

### 1-((6S,8aS)-6-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one & 1-((6R,8aR)-6-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one

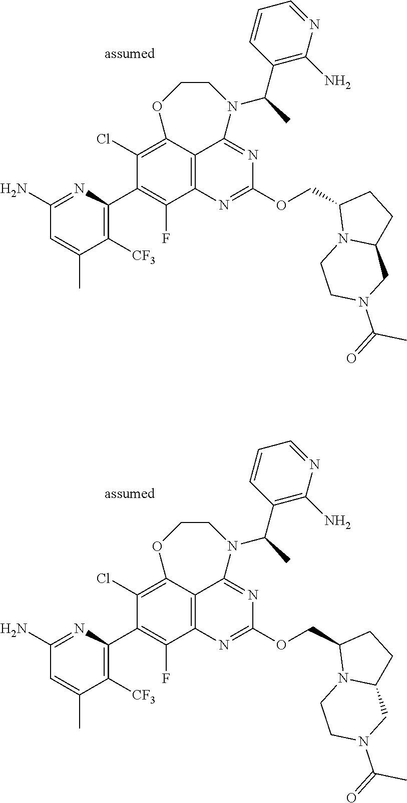

A solution of 1-((6S,8aS)-6-((((R)-9-(6-(bis(4-methoxybenzyl)amino)-4-methyl-3-(trifluoromethyl)pyridin-2-yl)-4-((R)-1-(2-(bis(4-methoxybenzyl)amino)pyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one (700.0 mg, 0.6 mmol) in trifluoroacetic acid (10 mL) and trifluoromethanesulfonic acid (1 mL) was stirred at 25° C. for 1 hour. After completion, the reaction mixture was adjusted to pH=7 with saturated sodium bicarbonate solution. The resulting solution was extracted with dichloromethane and the organic layers were combined. The organic layer was dried over anhydrous sodium sulfate and concentrated under vacuum. The residue was purified by flash chromatography on silica gel eluting with methanol/dichloromethane (1:5) to afford crude solid. The crude product was purified by Prep-HPLC with the following conditions (Column: XBridge Prep C18 OBD Column, 30*100 mm, 5 μm; Mobile Phase A: Water (10 mmol/L NH₄HCO3), Mobile Phase B: ACN; Flow rate: 60 mL/min; Gradient: 17% B to 42% B in 9 min, 42% B: Wave Length: 254/220 nm) to afford 50 mg product. The product was separated by Chiral-Prep-HPLC with the following conditions (Column: CHIRAL ART Cellulose-SC, 2*25 cm, 5 μm; Mobile Phase A: Hex:DCM=3:1(0.5% 2M NH3-MeOH)-HPLC, Mobile Phase B: IPA-HPLC; Flow rate: 20 mL/min; Gradient: 20% B to 20% B in 24 min; Wave Length: 220/254 nm) to afford 1-((6S,8aS)-6-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one (14.1 mg, 0.02 mmol, 3.3% yield) and 1-((6R,8aR)-6-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)hexahydropyrrolo[1,2-a]pyrazin-2(1H)-yl)ethan-1-one (21.0 mg, 0.03 mmol, 5% yield).

Example 123a: ¹H NMR (300 MHz, Methanol-d₄, ppm) δ ¹H NMR (300 MHz, Methanol-d₄, ppm) δ 7.96 (dd, J=5.1, 1.6 Hz, 1H), 7.75 (d, J=7.5 Hz, 1H), 6.78 (dd, J=7.5, 5.1 Hz, 1H), 6.59 (s, 1H), 6.50 (q, J=6.9 Hz, 1H), 4.53 (dd, J=11.1, 6.2 Hz, 1H), 4.48-4.32 (m, 2H), 4.34-4.06 (m, 2H), 3.88-3.59 (m, 3H), 3.59-3.34 (m, 2H), 3.29-2.95 (m, 3H), 2.95-2.56 (m, 1H), 2.44 (s, 3H), 2.31-2.15 (m, 1H), 2.10 (s, 3H), 2.01 (s, 1H), 1.88-1.71 (m, 1H), 1.66 (d, J=6.9 Hz, 3H), 1.60-1.40 (m, 1H). LC-MS: (ESI, m/z): 730.4 [M+H]⁺.

Chiral HPLC: Column: CHIRALPAK ID-3, 4.6*50 mm, 3 um; Mobile Phase: (Hex:DCM=3:1)(0.1% DEA):IPA=80:20; Flow: 1.0 mL/min: Retention time: 3.940 min (First peak).

Example 123b: ¹H NMR (300 MHz, Methanol-d₄, ppm) δ 7.96 (dd, J=5.1, 1.6 Hz, 1H), 7.75 (d, J=7.5 Hz, 1H), 6.78 (dd, J=7.5, 5.1 Hz, 1H), 6.59 (s, 1H), 6.50 (q, J=6.9 Hz, 1H), 4.53 (dd, J=11.1, 6.2 Hz, 1H), 4.48-4.32 (m, 2H), 4.34-4.06 (m, 2H), 3.88-3.59 (m, 3H), 3.59-3.34 (m, 2H), 3.29-2.95 (m, 3H), 2.95-2.56 (m, 1H), 2.44 (s, 3H), 2.31-2.15 (m, 1H), 2.10 (s, 3H), 2.01 (s, 1H), 1.88-1.71 (m, 1H), 1.66 (d, J=6.9 Hz, 3H), 1.60-1.40 (m, 1H). LC-MS: (ESI, m/z): 730.3 [M+H]⁺.

Chiral HPLC: Column: CHIRALPAK ID-3, 4.6*50 mm, 3 um; Mobile Phase: (Hex:DCM=3:1)(0.1% DEA):IPA=80:20; Flow: 1.0 mL/min; Retention time: 5.359 min (Second peak).

Example 124: 6-((R)-4-((R)-1-(2-aminopyridin-3-yl)ethyl)-8-chloro-10-fluoro-2-(((R)-4-methylmorpholin-2-yl)methoxy)-5,6-dihydro-4H-[1,4]oxazepino[5,6,7-de]quinazolin-9-yl)-4-methyl-5-(trifluoromethyl)pyridin-2-amine

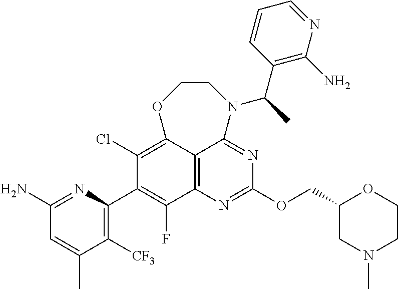

### Synthetic Route:

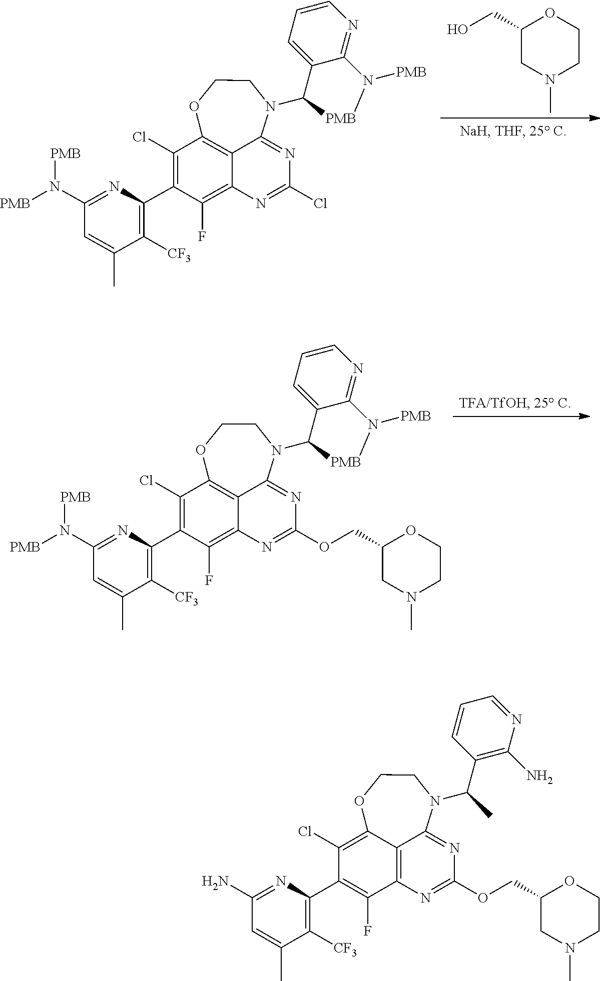

Step 1: (R)-6-(4-((R)-1-(2-(bis(4-methoxybenzyl)amino)pyridin-3-yl)ethyl)-8-chloro-10-fluoro-2-(((R)-4-methylmorpholin-2-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

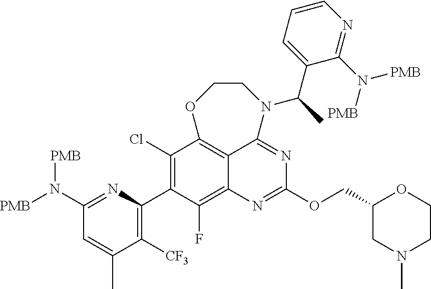

A solution of (R)-(4-methylmorpholin-2-yl)methanol (112.5 mg, 0.86 mmol) in tetrahydrofuran (4 mL) was added sodium hydride (46.0 mg, 1.15 mmol, 60% dispersion in mineral oil) at 0° C. and stirred at 25° C. for 10 minutes. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (300.0 mg, 0.29 mmol) was added and stirred at 25° C. for 8 hours. After completion, the resulting solution was diluted with ethyl acetate and washed with water. The organic layer was washed with brine and dried over anhydrous sodium sulfate and concentrated under vacuum. The residue was purified by flash chromatography on silica gel eluting with dichloromethane/methanol (9:1) to afford the title compound (112.0 mg, 0.10 mmol, 34.2% yield) as a yellow oil. LC-MS: (ESI, m/z): 1143.6 [M+H]⁺

Step 2: 6-((R)-4-((R)-1-(2-aminopyridin-3-yl)ethyl)-8-chloro-10-fluoro-2-(((R)-4-methylmorpholin-2-yl)methoxy)-5,6-dihydro-4H-[1,4]oxazepino[5,6,7-de]quinazolin-9-yl)-4-methyl-5-(trifluoromethyl)pyridin-2-amine

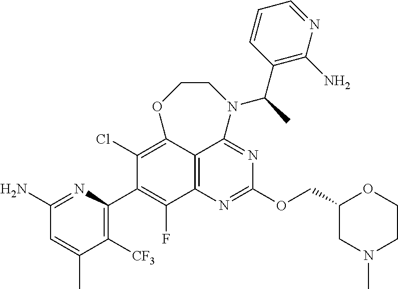

A solution of (R)-6-(4-((R)-1-(2-(bis(4-methoxybenzyl)amino)pyridin-3-yl)ethyl)-8-chloro-10-fluoro-2-(((R)-4-methylmorpholin-2-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (112.0 mg, 0.10 mmol) in trifluoromethanesulfonic acid (0.5 mL) and trifluoroacetic acid (5 mL) was stirred at 25° C. for 0.5 hour. After completion, the mixture was concentrated under vacuum. The residue was adjusted to pH=7 by saturated sodium bicarbonate solution. The mixture was diluted with dichloromethane and washed with water. The organic layer was washed with brine and dried over anhydrous sodium sulfate and concentrated under vacuum. The crude product was purified by Prep-HPLC with the following conditions: Column: YMC-Actus Triart C18 ExRS, 30*150 mm, 5 μm; Mobile Phase A: Water (10 mmol/L NH4HCO3), Mobile Phase B: ACN; Flow rate: 60 mL/min; Gradient: 17% B to 42% B in 9 min, 42% B: Wave Length: 254/220 nm; RT1(min): 8.6 to afford the title compound (40.8 mg, 0.062 mmol, 62.8% yield). LC-MS: (ESI, m/z): 663.1 [M+H]⁺

Example 124: ¹H NMR (300 MHz, DMSO-d₆, ppm) δ 7.97 (d, J=5.1, 1.6 Hz, 1H), 7.63 (d, J=7.3 Hz, 1H), 6.80 (s, 2H), 6.67 (dd, J=7.5, 4.9 Hz, 1H), 6.47 (s, 1H), 6.25 (d, J=6.8 Hz, 1H), 5.63 (s, 2H), 4.42 (dd, J=12.1, 6.4 Hz, 1H), 4.34 (d, J=5.1 Hz, 2H), 4.25 (dd, J=11.9, 7.3 Hz, 1H), 3.88-3.76 (m, 2H), 3.66-3.49 (m, 2H), 3.43-3.33 (m, 1H), 2.76 (d, J=11.3 Hz, 1H), 2.59 (d, J=11.3 Hz, 1H), 2.36 (s, 3H), 2.19 (s, 3H), 2.06-1.79 (m, 2H), 1.56 (d, J=6.7 Hz, 3H).

Example 125a & 125b: 6-((R)-4-((R)-1-(2-aminopyridin-3-yl)ethyl)-8-chloro-10-fluoro-2-(((6S,8aS)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((6R,8aR)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine

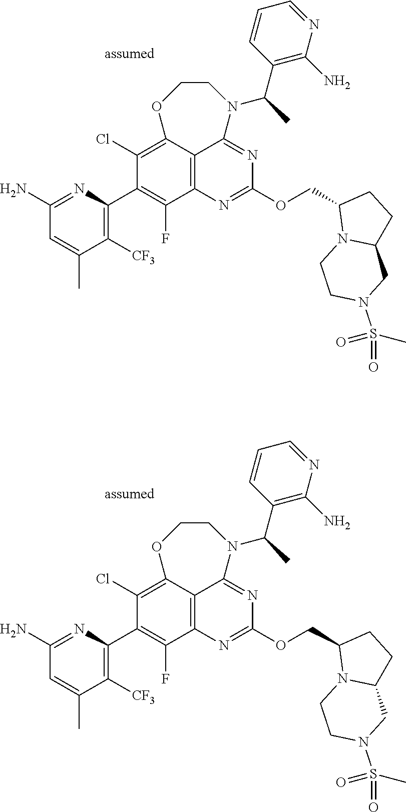

### Synthetic Route

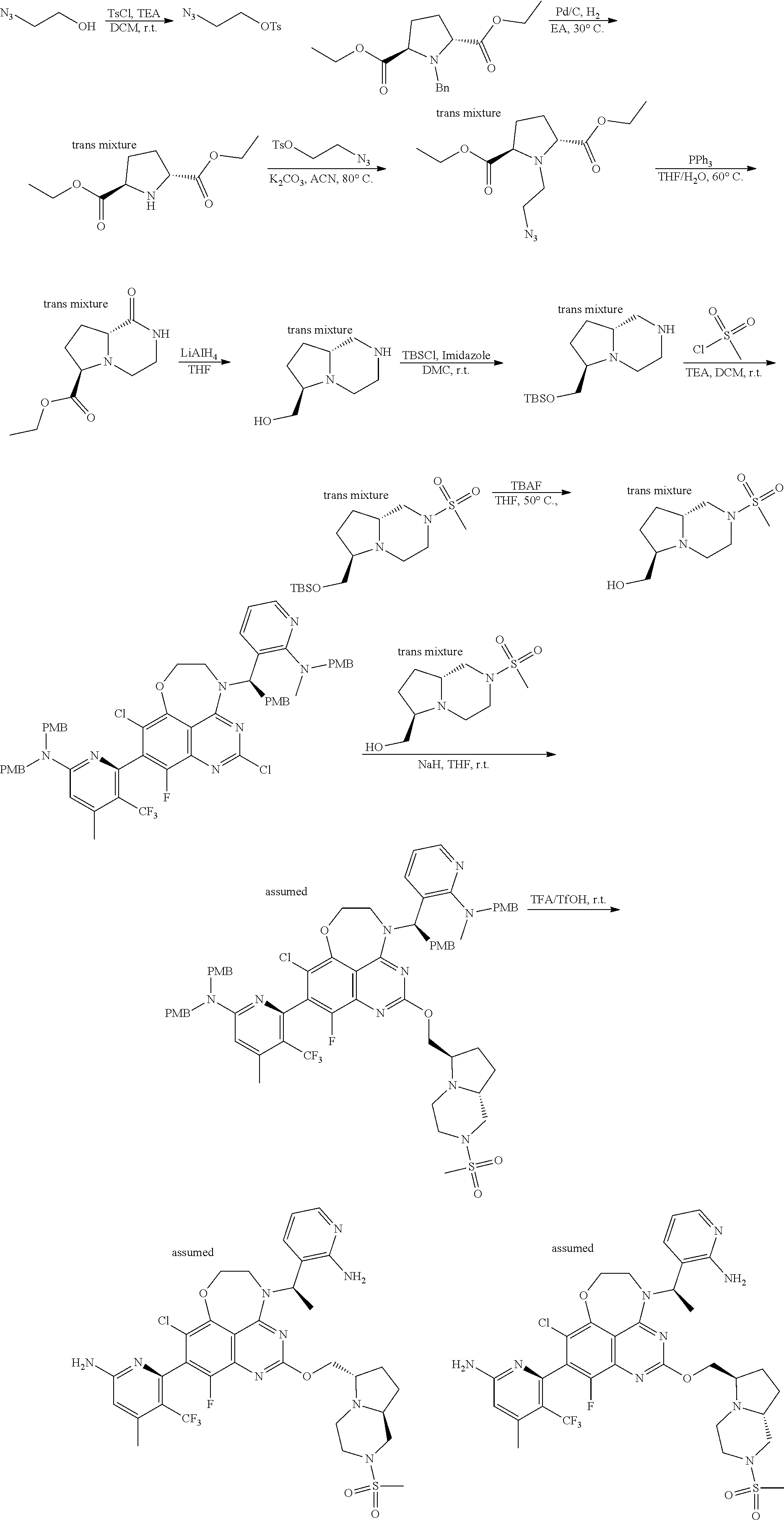

Step 1: 2-azidoethyl 4-methylbenzenesulfonate

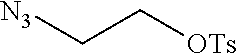 [SMILES: Cc1ccc(S(=O)(=O)OCCN=[N+]=[N-])cc1]

A solution of 2-azidoethanol (20.00 g, 229.6 mmol), tosyl chloride (66.00 g, 345.5 mmol) and triethylamine (46.00 g, 455.4 mmol) in dichloromethane (200 mL) was stirred at 25° C. for 2 hours. After completion, the reaction mixture was diluted with ethyl acetate, washed with saturated sodium chloride solution, dried over anhydrous sodium sulfate and concentrated under vacuum. The residue was purified by flash chromatography on silica gel eluting with petroleum ether/ethyl acetate (3:1) to afford 2-azidoethyl 4-methylbenzenesulfonate (25.00 g, 103.62 mmol, 45.1% yield) as a yellow oil.

Step 2: diethyl (2R,5R)-pyrrolidine-2,5-dicarboxylate

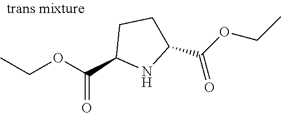

Under hydrogen, a solution of diethyl (2R,5R)-1-benzylpyrrolidine-2,5-dicarboxylate (20.00 g, 65.4 mmol) and Pd on charcoal (5 g, ca. 10% Pd)) in ethyl acetate (70 mL) was stirred at 30° C. for 5 hours at atmospheric pressure. After completion, the reaction mixture was filtered. The filtrate was collected and concentrated under vacuum. The product diethyl (2R,5R)-pyrrolidine-2,5-dicarboxylate (20.00 g, crude) was directly used in the next step without further purification. LC-MS: (ESI, m/z): 216.1 [M+H]⁺.

Step 3: diethyl (2R,5R)-1-(2-azidoethyl)pyrrolidine-2,5-dicarboxylate

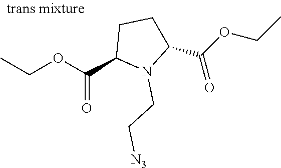 [SMILES: CCOC(=O)[C@H]1CC[C@H](C(=O)OCC)N1CCN=[N+]=[N-]]

A solution of diethyl (2R,5R)-pyrrolidine-2,5-dicarboxylate (4.01 g, 18.6 mmol), potassium carbonate (5.15 g, 37.3 mmol) and 2-azidoethyl 4-methylbenzenesulfonate (3.00 g, 12.4 mmol) in acetonitrile (30 mL) was stirred at 80° C. for 48 hours. After completion, the reaction mixture was filtered. The filtrate was collected and concentrated under vacuum. The residue was purified by flash chromatography on silica gel eluting with ethyl acetate/petroleum ether (1:10) to afford diethyl (2R,5R)-1-(2-azidoethyl)pyrrolidine-2,5-dicarboxylate (1.50 g, 5.2 mmol, 42.4% yield) as a colorless oil. LC-MS: (ESI, m/z): 285.1 [M+H]⁺.

Step 4: ethyl (6R,8aR)-1-oxooctahydropyrrolo[1,2-a]pyrazine-6-carboxylate

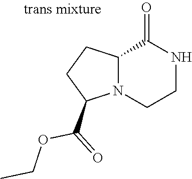

Under nitrogen, a solution of diethyl (2R,5R)-1-(2-azidoethyl)pyrrolidine-2,5-dicarboxylate (400.0 mg, 1.4 mmol), triphenylphosphine (369.0 mg, 1.4 mmol) and water (0.03 mL, 1.5 mmol) in tetrahydrofuran (4 mL) was stirred at 60° C. for 24 hours. After completion, the reaction mixture was filtered. The filtrate was collected and concentrated under vacuum. The residue was purified by flash chromatography on silica gel eluting with methanol/dichloromethane (1:30) to afford ethyl (6R,8aR)-1-oxooctahydropyrrolo[1,2-a]pyrazine-6-carboxylate (160.0 mg, 0.7 mmol, 53.6% yield) as a white solid. LC-MS: (ESI, m/z): 213.0 [M+H]⁺.

Step 5: ((6R,8aR)-octahydropyrrolo[1,2-a]pyrazin-6-yl)methanol

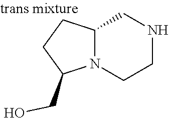 [SMILES: OC[C@H]1CC[C@@H]2CNCCN21]

A solution of ethyl (6R,8aR)-1-oxooctahydropyrrolo[1,2-a]pyrazine-6-carboxylate (150.0 mg, 0.7 mmol) in tetrahydrofuran (2 mL) was added lithium aluminum hydride (134.2 mg, 3.5 mmol) and stirred at 60° C. for 3 hours. After completion, the reaction was quenched with sodium sulfate decahydrate. The reaction mixture was diluted with tetrahydrofuran and filtered. The filtrate was collected and concentrated under vacuum to afford the title compound (120.0 mg, crude) which was directly used in the next step without further purification. LC-MS: (ESI, m/z): 157.1[M+H]⁺.

Step 6: (6R,8aR)-6-(((tert-butyldimethylsilyl)oxy)methyl)octahydropyrrolo[1,2-a]pyrazine

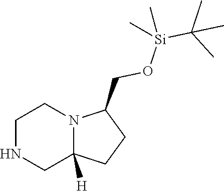 [SMILES: CC(C)(C)[Si](C)(C)OC[C@H]1CC[C@@H]2CNCCN21]

A solution of imidazole (915.0 mg, 13.4 mmol), ((6R,8aR)-octahydropyrrolo[1,2-a]pyrazin-6-yl)methanol (700.0 mg, 4.5 mmol) and tert-butyldimethylchlorosilane (1.35 g, 8.9 mmol) in dichloromethane (10 mL) was stirred at 25° C. for 2 hours. After completion, the reaction was diluted with ethyl acetate, washed with saturated brine, dried over anhydrous sodium sulfate and concentrated under vacuum to afford the title compound (1.40 g, crude) which was directly used in the next step without further purification. LC-MS: (ESI, m/z): 271.1 [M+H]⁺.

Step 7: (6R,8aR)-6-(((tert-butyldimethylsilyl)oxy)methyl)-2-(methylsulfonyl)octahydropyrrolo[1,2-a]pyrazine

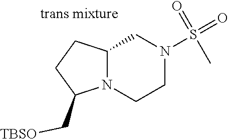 [SMILES: CC(C)(C)[Si](C)(C)OC[C@H]1CC[C@@H]2CN(S(C)(=O)=O)CCN12]

A solution of (6R,8aR)-6-(((tert-butyldimethylsilyl)oxy)methyl)octahydropyrrolo[1,2-a]pyrazine (490.0 mg, crude), methanesulfonyl chloride (124.5 mg, 1.1 mmol) and triethylamine (329.9 mg, 3.3 mmol) in dichloromethane (5 mL) was stirred at 25° C. for 2 hours. After completion, the reaction mixture was diluted with dichloromethane, washed with water, dried over anhydrous sodium sulfate and concentrated under vacuum. The residue was purified by flash chromatography on silica gel eluting with methanol/dichloromethane (1:30) to afford the title compound (300.0 mg, 0.9 mmol, 79.1% yield) as a yellow oil. LC-MS: (ESI, m/z): 349.0 [M+H]⁺.

Step 8: ((6R,8aR)-2-(methylsulfonyl)octahydropyrrolo[1,2-a]pyrazin-6-yl)methanol

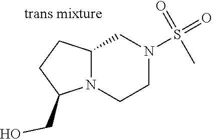

A solution of (6R,8aR)-6-(((tert-butyldimethylsilyl)oxy)methyl)-2-(methylsulfonyl)octahydropyrrolo[1,2-a]pyrazine (260.0 mg, 0.7 mmol) and tetrabutylammonium fluoride (390.0 mg, 1.5 mmol) in tetrahydrofuran (3 mL) was stirred at 50° C. for 2 hours. After completion, the reaction mixture was concentrated under reduced pressure. The residue was purified by flash chromatography on silica gel eluting with dichloromethane/methanol (30:1) to afford the title compound (150.0 mg, 0.6 mmol, 85.8% yield) as a colorless oil. LC-MS: (ESI, m/z): 235.0 [M+H]⁺.

Step 9: 6-((R)-4-((R)-1-(2-(bis(4-methoxybenzyl)amino)pyridin-3-yl)ethyl)-8-chloro-10-fluoro-2-(((6R,8aR)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

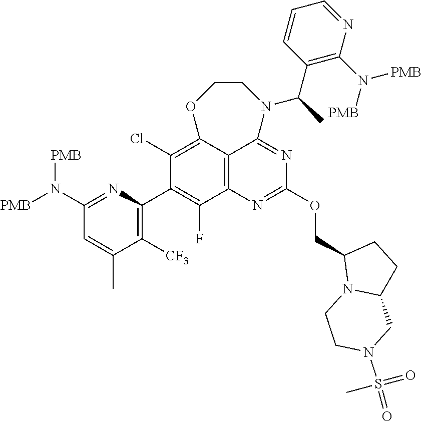

A solution of ((6R,8aR)-2-(methylsulfonyl)octahydropyrrolo[1,2-a]pyrazin-6-yl)methanol (150.1 mg, 0.6 mmol) in tetrahydrofuran (4 mL) was added sodium hydride (68.3 mg, 1.7 mmol, 60% purity) and stirred at 0° C. for 15 min. Then 6-((R)-4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (448.0 mg, 0.4 mmol) in tetrahydrofuran (4 mL) was added and stirred at 30° C. for 24 hours. After completion, the reaction was quenched with saturated ammonium chloride solution. The reaction mixture was diluted with ethyl acetate, washed with water, dried over anhydrous sodium sulfate and concentrated under vacuum. The residue was purified by flash chromatography on silica gel eluting with dichloromethane/methanol (10:1) to afford the title compound (190.0 mg, 0.2 mmol, 35.7% yield) as a white solid. LC-MS: (ESI, m/z): 1246.1 [M+H]⁺.

Step 10: 6-((R)-4-((R)-1-(2-aminopyridin-3-yl)ethyl)-8-chloro-10-fluoro-2-(((6S,8aS)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine& 6-((R)-4-((R)-1-(2-aminopyridin-3-yl)ethyl)-8-chloro-10-fluoro-2-(((6R,8aR)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine

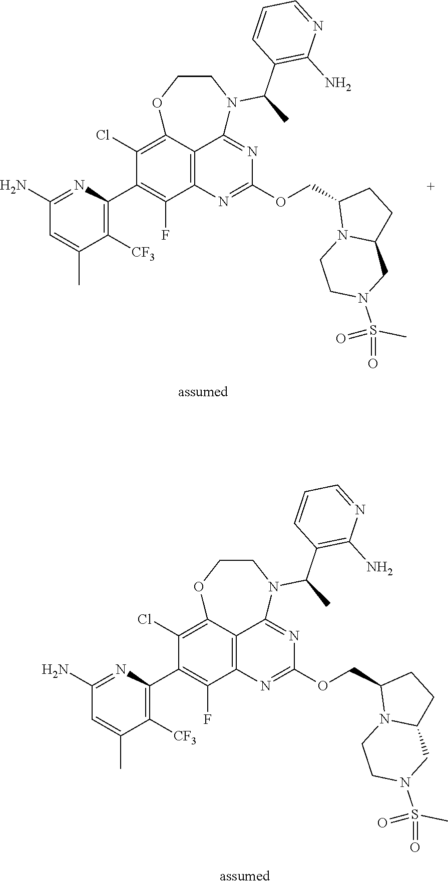

A solution of 6-((R)-4-((R)-1-(2-(bis(4-methoxybenzyl)amino)pyridin-3-yl)ethyl)-8-chloro-10-fluoro-2-(((6R,8aR)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (180.0 mg, 0.1 mmol) in trifluoroacetic acid (0.3 mL) and trifluoromethanesulfonic acid (0.03 mL) was stirred at 25° C. for 1 hour. After completion, the reaction mixture was concentrated under reduced pressure. The residue was purified by flash chromatography on reversed-phase C18 column acetonitrile/water (85:15) to afford crude product. The crude product was purified by Prep-HPLC with the following conditions (Column: XBridge Shield RP18 OBD Column, 30*150 mm, 5 μm; Mobile Phase A: Water (10 mmol/L NH4HCO₃), Mobile Phase B: ACN; Flow rate: 60 mL/min; Gradient: 36% B to 54% B in 8 min, 54% B; Wave Length: 254/220 nm;) to afford 70 mg product. The product was separated by Chiral-Prep-HPLC with the following conditions (Column: CHIRALPAK ID, 2*25 cm, 5 μm; Mobile Phase A: Hex:DCM=3:1(0.5% 2M NH₃-MeOH)-HPLC, Mobile Phase B: IPA-HPLC; Flow rate: 20 mL/min; Gradient: 30% B to 30% B in 26 min; Wave Length: 220/254 nm) to afford 6-((R)-4-((R)-1-(2-aminopyridin-3-yl)ethyl)-8-chloro-10-fluoro-2-(((6S,8aS)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine (22.8 mg, 0.03 mmol, 20.6% yield) and 6-((R)-4-((R)-1-(2-aminopyridin-3-yl)ethyl)-8-chloro-10-fluoro-2-(((6R,8aR)-2-(methylsulfonyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine (21.4 mg, 0.03 mmol, 19.3% yield).

Example 125a: ¹H NMR (300 MHz, DMSO-d₄, ppm) δ 7.98 (dd, J=4.9, 1.7 Hz, 1H), 7.64 (dd, J=7.4, 1.8 Hz, 1H), 6.80 (s, 2H), 6.71-6.57 (m, 1H), 6.48 (s, 1H), 6.24 (q, J=6.8 Hz, 1H), 5.70 (s, 2H), 4.53-4.34 (m, 2H), 4.32-4.12 (m, 2H), 3.74-3.46 (m, 2H), 3.45-3.37 (m, 1H), 3.26-3.13 (m, 2H), 3.09-2.95 (m, 1H), 2.94-2.72 (m, 5H), 2.49-2.42 (m, 1H), 2.41-2.31 (m, 3H), 2.15-1.85 (m, 2H), 1.74-1.62 (m, 1H), 1.57 (d, J=6.8 Hz, 3H), 1.48-1.32 (m, 1H), 1.21 (d, J=17.6 Hz, 1H). LC-MS: (ESI, m/z): 766.2 [M+H]⁺. Chiral HPLC: Column: CHIRALPAK ID-3, 4.6*50 mm, 3 um; Mobile Phase: (Hex:DCM=3:1)(0.1% DEA):IPA=70:30; Flow: 1.0 mL/min; Retention time: 2.406 min (First peak).

Example 125b: ¹H NMR (300 MHz, DMSO-d₆, ppm) δ 7.97 (dd, J=4.9, 1.7 Hz, 1H), 7.63 (dd, J=7.5, 1.8 Hz, 1H), 6.80 (s, 2H), 6.67 (dd, J=7.5, 4.9 Hz, 1H), 6.48 (s, 1H), 6.26 (q, J=6.6 Hz, 1H), 5.75 (s, 2H), 4.51-4.34 (m, 2H), 4.33-4.04 (m, 2H), 3.75-3.48 (m, 2H), 3.46-3.36 (m, 1H), 3.28-3.13 (m, 2H), 3.11-2.98 (m, 1H), 2.94-2.67 (m, 5H), 2.48-2.41 (m, 1H), 2.40-2.30 (m, 3H), 2.18-1.80 (m, 2H), 1.72-1.63 (m, 1H), 1, 57 (d, J=6.8 Hz, 3H), 1.46-1.30 (m, 1H), 1.29-1.17 (m, 1H). LC-MS: (ESI, m/z): 766.2 [M+H]⁺. Chiral HPLC: Column: CHIRALPAK ID-3, 4.6*50 mm, 3 um; Mobile Phase: (Hex:DCM=3:1)(0.1% DEA):IPA=70:30; Flow: 1.0 mL/min; Retention time: 3.016 min (Second peak).

Example 126: 6-((R)-4-((R)-1-(2-aminopyridin-3-yl)ethyl)-8-chloro-10-fluoro-2-((1-(2-methoxyethyl)piperidin-4-yl)oxy)-5,6-dihydro-4H-[1,4]oxazepino[5,6,7-de]quinazolin-9-yl)-4-methyl-5-(trifluoromethyl)pyridin-2-amine

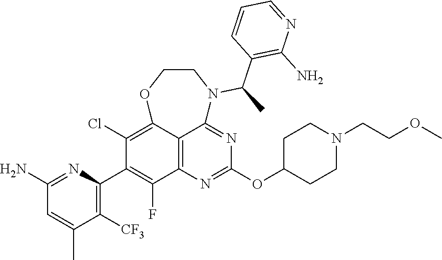

### Synthetic Route

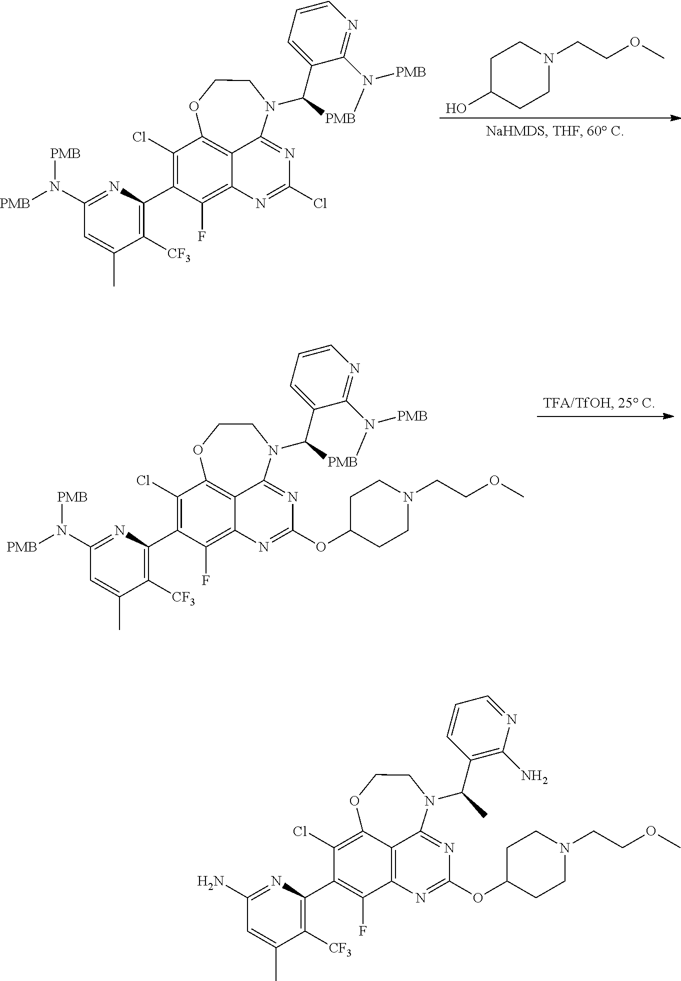

Step 1: (R)-6-(4-((R)-1-(2-(bis(4-methoxybenzyl)amino)pyridin-3-yl)ethyl)-8-chloro-10-fluoro-2-((1-(2-methoxyethyl)piperidin-4-yl)oxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

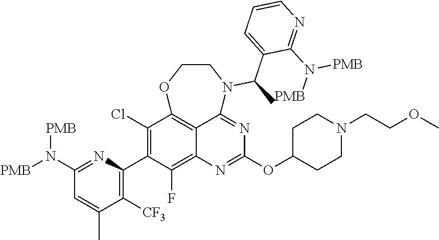

Under nitrogen, a solution of 1-(2-methoxyethyl)piperidin-4-ol (319.0 mg, 2 mmol) in tetrahydrofuran (3 mL) was added sodium bis(trimethylsilyl)amide (2.8 mL, 2.8 mmol, 1 M in tetrahydrofuran) and stirred at 25° C. for 10 minutes. Then (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (700.0 mg, 0.67 mmol) was added and stirred at 60° C. for 4 hours. The resulting solution was diluted with water and extracted with ethyl acetate. Then the organic layers were washed with brine and dried over anhydrous sodium sulfate and concentrated under vacuum. The residue was purified by flash chromatography on silica gel eluting with dichloromethane/methanol (10:1) to afford the title compound (310.0 mg, 0.26 mmol, 39.6% yield) as a yellow solid. LC-MS: (ESI, m/z): 1171.5 [M+H]⁺

Step 2: 6-((R)-4-((R)-1-(2-aminopyridin-3-yl)ethyl)-8-chloro-10-fluoro-2-((1-(2-methoxyethyl)piperidin-4-yl)oxy)-5,6-dihydro-4H-[1,4]oxazepino[5,6,7-de]quinazolin-9-yl)-4-methyl-5-(trifluoromethyl)pyridin-2-amine

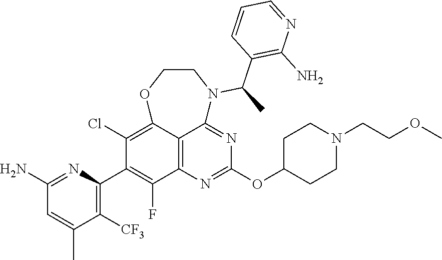

A solution of (R)-6-(4-((R)-1-(2-(bis(4-methoxybenzyl)amino)pyridin-3-yl)ethyl)-8-chloro-10-fluoro-2-((1-(2-methoxyethyl)piperidin-4-yl)oxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (270.0 mg, 0.23 mmol) in trifluoromethanesulfonic (0.3 mL) and trifluoroacetic acid (3.0 mL) was stirred at 25° C. for 0.5 hour. After completion, the solvent was concentrated under vacuum. The residue was purified by Prep-HPLC with the following conditions: Column: XSelect CSH Fluoro Phenyl, 30*150 mm, 5 μm; Mobile Phase A: Water (10 mmol/L NH4HCO₃), Mobile Phase B: ACN; Flow rate: 60 mL/min; Gradient: 46% B to 71% B in 9 min, 71% B; Wave Length: 254/220 nm; RT1(min): 6.52 to afford the title compound (74.3 mg, 0.10 mmol, 45.7% yield). LC-MS: (ESI, m/z): 691.2 [M+H]⁺

Example 126: ¹H NMR (400 MHz, DMSO-d6, ppm) δ 7.97 (dd, J=4.9, 1.7 Hz, 1H), 7.60 (dd, J=7.6, 1.8 Hz, 1H), 6.79 (s, 2H), 6.66 (dd, J=7.5, 4.9 Hz, 1H), 6.47 (s, 1H), 6.18 (q, J=6.8 Hz, 1H), 5.64 (s, 2H), 5.01 (dt, J=8.7, 4.5 Hz, 1H), 4.42-4.40 (m, 1H), 4.29-4.27 (m, 1H), 3.69-3.58 (m, 1H), 3.46-3.33 (m, 3H), 3.23 (s, 3H), 2.76-2.73 (m, 2H), 2.49-2.45 (m, 2H), 2.36 (s, 3H), 2.25 (q, J=10.7 Hz, 2H), 1.99-1.96 (m, 2H), 1.78-1.61 (m, 2H), 1.56 (d, J=6.8 Hz, 3H).

Example 127: 6-((R)-4-((R)-1-(2-aminopyridin-3-yl)ethyl)-8-chloro-10-fluoro-2-(((S)-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

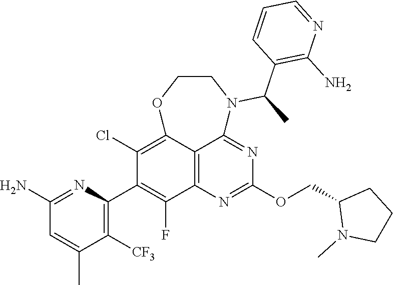

### Synthetic Route:

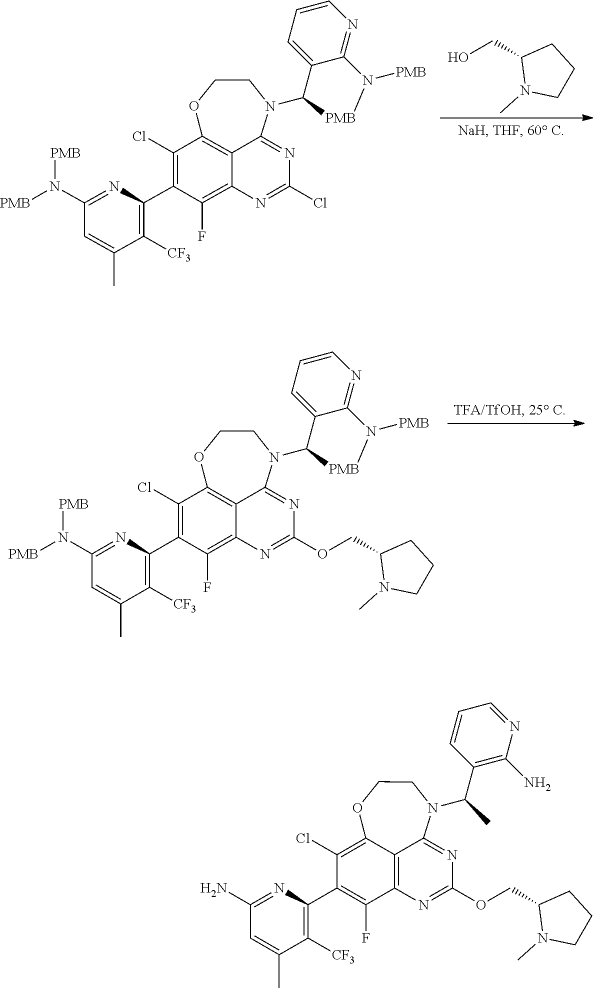

Step 1: (R)-6-(4-((R)-1-(2-(bis(4-methoxybenzyl)amino)pyridin-3-yl)ethyl)-8-chloro-10-fluoro-2-(((S)-1-methylpyrrolidin-2-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

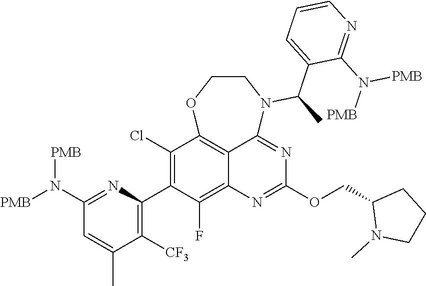

To a solution of (S)-(1-methylpyrrolidin-2-yl)methanol (62.0 mg, 0.5400 mmol) in tetrahydrofuran (2 mL) was added sodium hydride (38.0 mg, 0.95 mmol, 60% dispersion in mineral oil) and stirred at 0° C. for 10 minutes. Then a solution of (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (200.0 mg, 0.1900 mmol) was added and stirred at 60° C. for 1 hour. After completion, the reaction was quenched with saturated ammonium chloride solution at 0° C. The reaction mixture was diluted with water. The resulting solution was extracted with ethyl acetate and the organic layers were combined. The organic layer was dried over anhydrous sodium sulfate and concentrated under vacuum. The residue was purified by flash chromatography on silica gel eluting with dichloromethane/methanol (7:1) to afford the title compound (150 mg, 0.13 mmol, 69.8% yield) as a yellow solid. LC-MS: (ESI, m/z): 1127.7 [M+H]⁺

Step 2: 6-((R)-4-((R)-1-(2-aminopyridin-3-yl)ethyl)-8-chloro-10-fluoro-2-(((S)-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

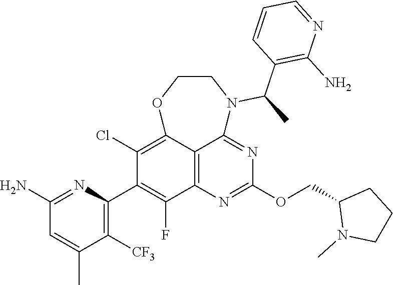

A solution of (R)-6-(4-((R)-1-(2-(bis(4-methoxybenzyl)amino)pyridin-3-yl)ethyl)-8-chloro-10-fluoro-2-(((S)-1-methylpyrrolidin-2-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (150.0 mg, 0.1300 mmol) in trifluoroacetic acid (3.0 mL) and trifluoromethanesulfonic (0.3 mL) was stirred at 25° C. for 0.5 hour. After completion, the solvent was concentrated under vacuum. The residue was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min: Gradient: 23% B to 43% B in 11 min, 43% B; Wave Length: 254/220 nm; RT1(min): 10.72 to afford the title compound (42.7 mg, 0.066 mmol, 49.6% yield). LC-MS: (ESI, m/z): 647.1 [M+H]⁺

Example 127: ¹H NMR (300 MHz, DMSO-d₆, ppm) δ 7.97 (dd, J=4.9, 1.7 Hz, 1H), 7.64 (dd, J=7.5, 1.8 Hz, 1H), 6.81 (s, 2H), 6.67 (dd, J=7.5, 4.9 Hz, 1H), 6.48 (s, 1H), 6.26 (d, J=6.9 Hz, 1H), 5.67 (s, 2H), 4.45-4.37 (m, 2H), 4.29-4.15 (m, 2H), 3.67 (dd, J=15.5, 6.8 Hz, 1H), 3.38 (d, J=6.8 Hz, 1H), 2.96 (dd, J=7.4, 3.8 Hz, 1H), 2.67-2.55 (m, 1H), 2.37 (s, 6H), 2.19 (q, J=8.4 Hz, 1H), 1.96-1.93 (m, 1H), 1.71-1.63 (m, 3H), 1.57 (d, J=6.8 Hz, 3H).

Example 128a & 128b: 6-((R)-4-((R)-1-(2-aminopyridin-3-yl)ethyl)-8-chloro-10-fluoro-2-(((R)-tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-4-methyl-5-(trifluoromethyl)pyridin-2-amine& 6-((R)-4-((R)-1-(2-aminopyridin-3-yl)ethyl)-8-chloro-10-fluoro-2-(((S)-tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-4-methyl-5-(trifluoromethyl)pyridin-2-amine

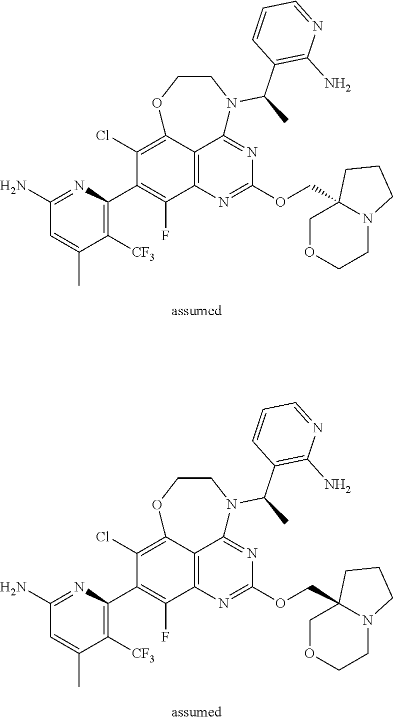

### Synthetic Route

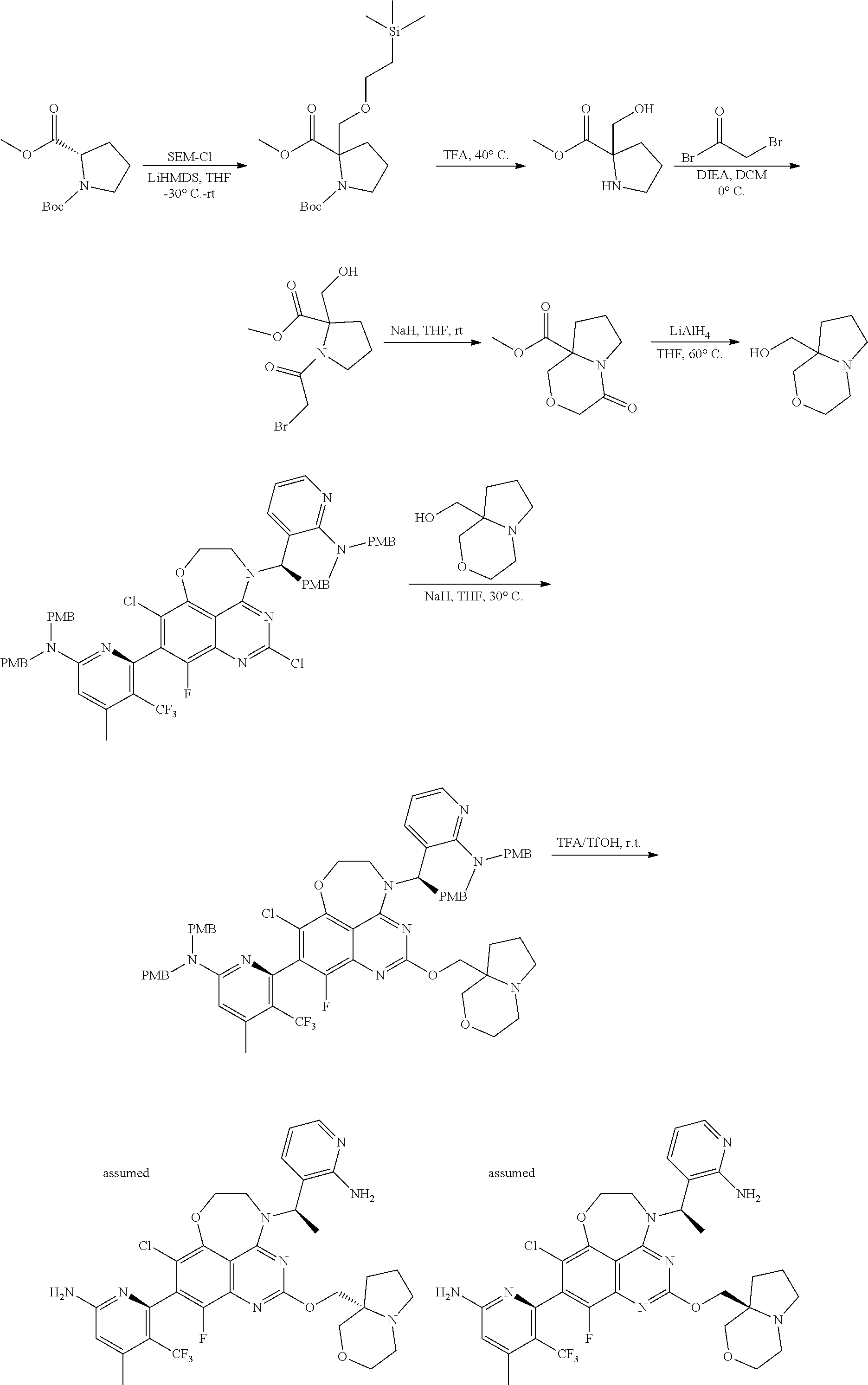

Step 1: 1-(tert-butyl) 2-methyl 2-((2-(trimethylsilyl)ethoxy)methyl)pyrrolidine-1,2-dicarboxylate

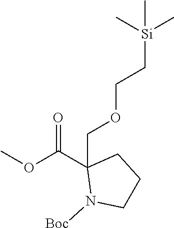

Under nitrogen, a solution of 1-(ted-butyl) 2-methyl (S)-pyrrolidine-1,2-dicarboxylate (20.00 g, 87.3 mmol) in tetrahydrofuran (150 mL) was added lithium bis(trimethylsilyl)amide (139 mL, 139.5 mmol, 1M in tetrahydrofuran) and stirred for 1 hour at −30° C. Then (2-(chloromethoxy)ethyl)trimethylsilane (21.85 g, 130.8 mmol) was dropwise added and stirred at 25° C. for 2 hours. After completion, the reaction was quenched with saturated ammonium chloride solution. The reaction mixture was diluted with ethyl acetate, washed with water, dried over anhydrous sodium sulfate and concentrated under vacuum. The residue was purified by flash chromatography on silica gel eluting with ethyl acetate/petroleum ether (1:8) to afford the title compound (30.00 g, 83.4 mmol, 95.7% yield) as a yellow oil. LC-MS: (ESI, m/z): 360.2 [M+H]⁺.

Step 2: methyl 2-(hydroxymethyl)pyrrolidine-2-carboxylate

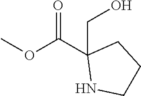

A solution of 1-(tert-butyl) 2-methyl 2-((2-(trimethylsilyl)ethoxy)methyl)pyrrolidine-1,2-dicarboxylate (3.00 g, 8.3 mmol) in trifluoroacetic acid (25 mL) was stirred at 40° C. for 20 hours. After completion, the reaction mixture was concentrated under reduced pressure. The residue was purified by reverse phase eluting with acetonitrile/water (0.1% ammonium bicarbonate) (30%) to afford methyl 2-(hydroxymethyl)pyrrolidine-2-carboxylate (2.00 g, crude) as a yellow oil. LC-MS: (ESI, m/z): 160.0 [M+H]⁺.

Step 3: methyl 1-(2-bromoacetyl)-2-(hydroxymethyl)pyrrolidine-2-carboxylate

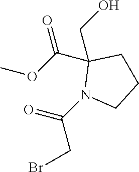

A solution of methyl 2-(hydroxymethyl)pyrrolidine-2-carboxylate (2.30 g, 14.4 mmol) and N,N-diisopropylethylamine (5.59 g, 43.3 mmol) in dichloromethane (20 mL) was stirred at 40° C. for 20 min. Then 2-bromoacetyl bromide (0.9 mL, 10.1 mmol) was dropwise added and stirred at 0° C. for 3 hours. After completion, the reaction mixture was concentrated under vacuum. The residue was purified by flash chromatography on silica gel eluting with ethyl acetate/petroleum ether (2:1) to afford the title compound (1.00 g, 3.5 mmol, 24.7% yield) as a yellow oil. LC-MS: (ESI, m/z): 280.0 [M+H]⁺.

Step 4: methyl 4-oxotetrahydro-1H-pyrrolo[2,1-c][1,4]oxazine-8a(6H)-carboxylate

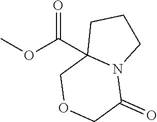

A solution of 1-(2-bromoacetyl)-2-(hydroxymethyl)pyrrolidine-2-carboxylate (1.00 g, 3.6 mmol) in tetrahydrofuran (8 mL) was added sodium hydride (285.6 mg, 7.1 mmol, 60% purity) and stirred at 0° C. for 1 hour. Then the reaction was stirred at 25° C. for 2 hours. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with dichloromethane/methanol (10:1) to afford methyl 4-oxo-1,6,7,8-tetrahydropyrrolo[2,1-c][1,4]oxazine-8a-carboxylate (300.0 mg, 1.5 mmol, 42.2% yield) as an yellow oil. LC-MS: (ESI, m/z): 200.2 [M+H]⁺.

Step 5: (tetrahydro-1H-pyrrolo[2,1-c][1,4]oxazin-8a(6H)-yl)methanol

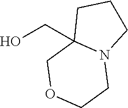 [SMILES: OCC12CCCN1CCOC2]

A solution of methyl 4-oxotetrahydro-1H-pyrrolo[2,1-c][1,4]oxazine-8a(6H)-carboxylate (300.0 mg, 1.5 mmol) in tetrahydrofuran (2 mL) was added lithium aluminum hydride (286.1 mg, 7.5 mmol) and stirred at 60° C. for 1 hour. After completion, the reaction was quenched with sodium sulfate decahydrate at 0° C. The resulting mixture was filtered, the filter cake was washed with dichloromethane. The filtrate was collected and concentrated under vacuum. The residue was purified by flash chromatography on silica gel eluting with methanol/dichloromethane (1:5) to afford the title compound (50.0 mg, 0.3 mmol, 21.1% yield) as a light yellow oil. LC-MS: (ESI, m/z): 158.1 [M+H]⁺.

Step 6: (6R)-6-(4-((R)-1-(2-(bis(4-methoxybenzyl)amino)pyridin-3-yl)ethyl)-8-chloro-10-fluoro-2-((tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

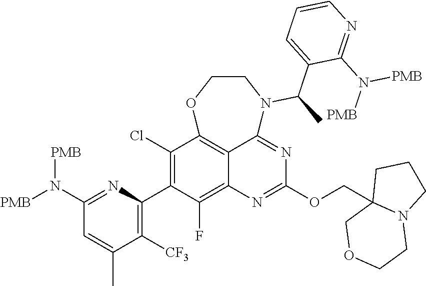

A solution of (tetrahydro-1H-pyrrolo[2,1-c][1,4]oxazin-8a(6H)-yl)methanol (48.5 mg, 0.3 mmol) in tetrahydrofuran (2 mL) was added sodium hydride (30.8 mg, 0.7 mmol, 60% purity) and stirred at 0° C. for 15 minutes. Then the reaction solution was transferred into a solution (R)-6-(4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (270.0 mg, 0.2 mmol) in tetrahydrofuran (5 mL) was added and stirred at 25° C. for 6 hours. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with ethyl acetate/petroleum ether (1:8) to afford the title compound (120.0 mg, 0.1 mmol, 39.9% yield) as a white solid. LC-MS: (ESI, m/z): 1169.5 [M+H]⁺.

Step 7

### 6-((R)-4-((R)-1-(2-aminopyridin-3-yl)ethyl)-8-chloro-10-fluoro-2-(((R)-tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-4-methyl-5-(trifluoromethyl)pyridin-2-amine &

### 6-((R)-4-((R)-1-(2-aminopyridin-3-yl)ethyl)-8-chloro-10-fluoro-2-(((S)-tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-4-methyl-5-(trifluoromethyl)pyridin-2-amine

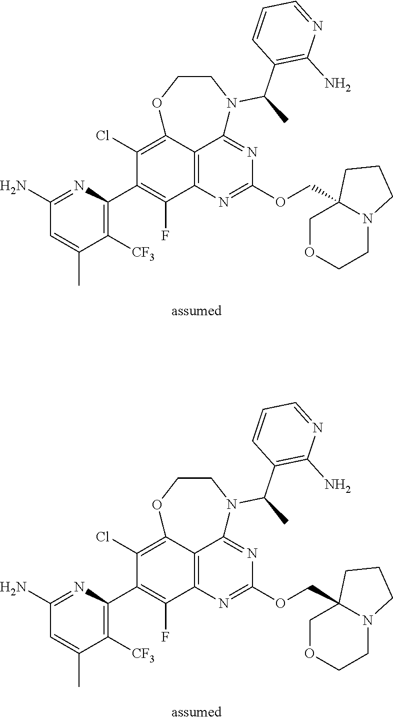

A solution of (6R)-6-(4-((R)-1-(2-(bis(4-methoxybenzyl)amino)pyridin-3-yl)ethyl)-8-chloro-10-fluoro-2-((tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (100.0 mg, 0.1 mmol) in trifluoroacetic acid (1 mL) and trifluoromethanesulfonic acid (0.1 mL) was stirred at 25° C. for 1 hour. After completion, the reaction mixture was adjusted to pH=7 with saturated sodium bicarbonate solution at 0° C. The resulting solution was extracted with dichloromethane and the organic layers were combined. The organic layer was dried over anhydrous sodium sulfate and concentrated under vacuum. The residue was purified by flash chromatography on silica gel eluting with methanol/dichloromethane (1:10) to afford crude solid. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 27% B to 52% B in 9 min, 52% B; Wave Length: 254/220 nm) to afford 13 mg product. The product was separated by Chiral-Prep-HPLC with the following conditions (Column: CHIRAL ART Amylose-SA, 2*25 cm, 5 μm; Mobile Phase A: Hex:DCM=3:1(0.5% 2M NH3-MeOH)-HPLC, Mobile Phase B: IPA-HPLC; Flow rate: 20 mL/min: Gradient: 10% B to 10% B in 21 min; Wave Length: 220/254 nm) to afford 6-((R)-4-((R)-1-(2-aminopyridin-3-yl)ethyl)-8-chloro-10-fluoro-2-(((R)-tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-4-methyl-5-(trifluoromethyl)pyridin-2-amine (6.4 mg, 0.009 mmol, 10.8% yield) and 6-((R)-4-((R)-1-(2-aminopyridin-3-yl)ethyl)-8-chloro-10-fluoro-2-(((S)-tetrahydro-1H-pyrrolo[2,1-c][1,4]oxazin-8a(6H)-yl)methoxy)-5,6-dihydro-4H-[1,4]oxazepino[5,6,7-de]quinazolin-9-yl)-4-methyl-5-(trifluoromethyl)pyridin-2-amine (1.0 mg, 0.001 mmol, 1.7% yield).

Example 128a: ¹H NMR (300 MHz, Methanol-d₄, ppm) δ 7.98 (dd, J=5.1, 1.7 Hz, 1H), 7.78 (d, J=7.6 Hz, 1H), 6.80 (dd, J=7.5, 5.1 Hz, 1H), 6.67-6.46 (m, 2H), 4.90-4.83 (s, 1H), 4.51-4.35 (m, 2H), 4.34-4.20 (m, 1H), 3.93-3.76 (m, 2H), 3.74-3.61 (m, 1H), 3.61-3.42 (m, 3H), 3.28-3.16 (m, 2H), 3.11-2.94 (m, 1H), 2.86-2.68 (m, 1H), 2.46 (s, 3H), 2.10-1.85 (m, 3H), 1.79-1.54 (m, 4H). LC-MS: (ESI, m/z): 689.4 [M+H]⁺. Chiral HPLC: Column: CHIRALPAK IA-3, 4.6*50 mm, 3 um; Mobile Phase: (Hex:DCM=3:1)(0.1% DEA):IPA=90:10; Flow: 1.0 mL/min; Retention time: 2.238 min (First peak).

Example 128b: ¹H NMR (300 MHz, Methanol-d₄, ppm) δ 7.98 (dd, J=5.1, 1.7 Hz, 1H), 7.78 (d, J=7.6 Hz, 1H), 6.80 (dd, J=7.5, 5.1 Hz, 1H), 6.67-6.46 (m, 2H), 4.90-4.83 (s, 1H), 4.51-4.35 (m, 2H), 4.34-4.20 (m, 1H), 3.93-3.76 (m, 2H), 3.74-3.61 (m, 1H), 3.61-3.42 (m, 3H), 3.28-3.16 (m, 2H), 3.11-2.94 (m, 1H), 2.86-2.68 (m, 1H), 2.46 (s, 3H), 2.10-1.85 (m, 3H), 1.79-1.54 (m, 4H). LC-MS: (ESI, m/z): 689.4 [M+H]⁺. Chiral HPLC: Column: CHIRALPAK IA-3, 4.6*50 mm, 3 um: Mobile Phase: (Hex:DCM=3:1)(0.1% DEA):IPA=90:10; Flow: 1.0 mL/min; Retention time: 3.624 min (Second peak).

Example 129a & 129b: 6-((R)-4-((R)-1-(2-aminopyridin-3-yl)ethyl)-8-chloro-10-fluoro-2-(((6S,8aS)-2-(2,2,2-trifluoroethyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((6R,8aR)-2-(2,2,2-trifluoroethyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine

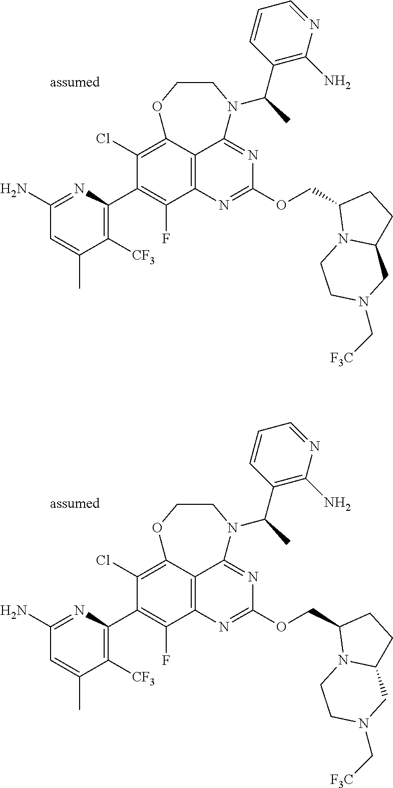

### Synthetic Route

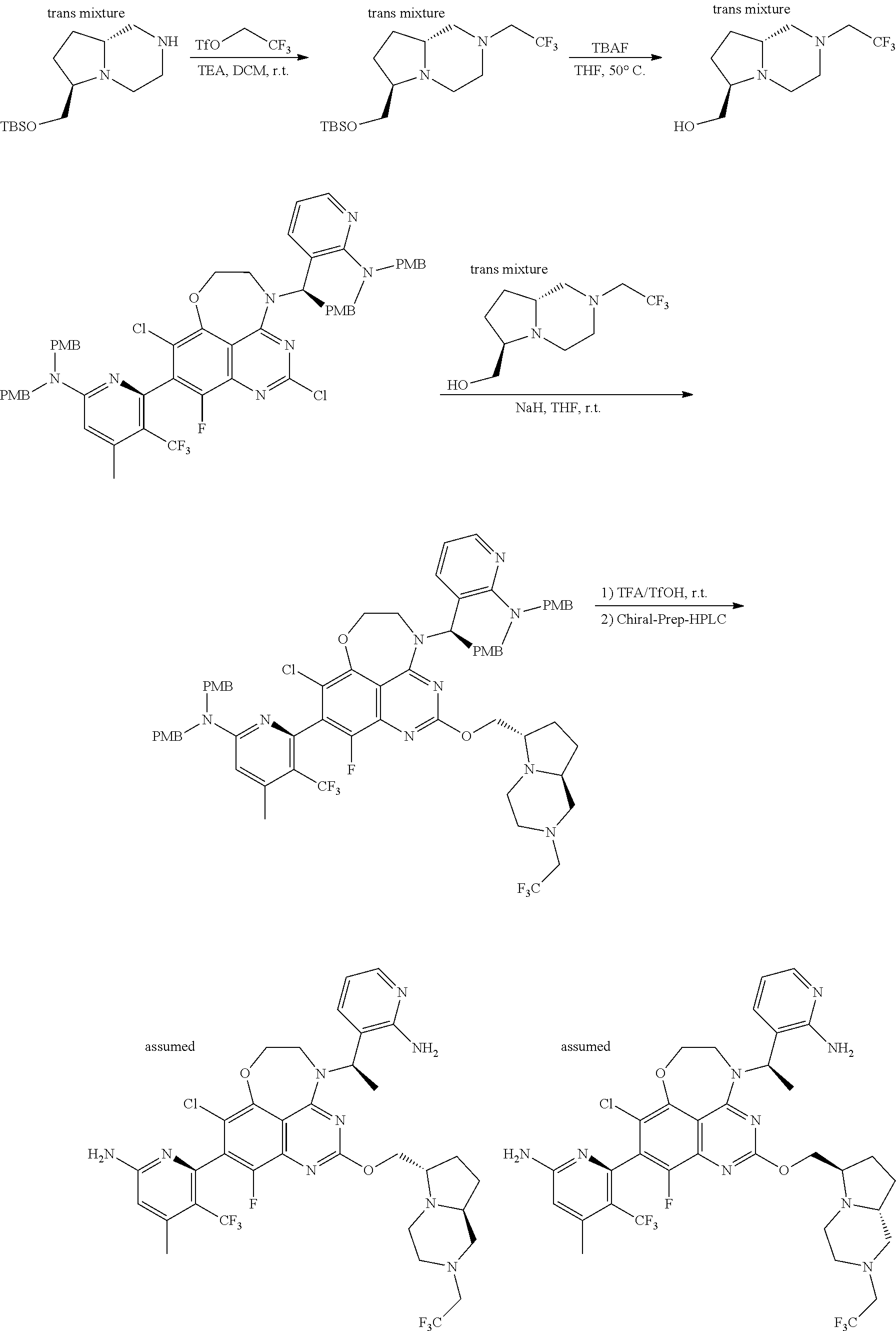

Step 1: (6R,8aR)-6-(((tert-butyldimethylsilyl)oxy)methyl)-2-(2,2,2-trifluoroethyl)octahydropyrrolo[1,2-a]pyrazine

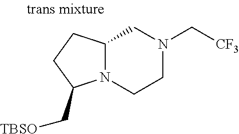 [SMILES: CC(C)(C)[Si](C)(C)OC[C@H]1CC[C@@H]2CN(CC(F)(F)F)CCN12]

A solution of (6R,8aR)-6-(((tert-butyldimethylsilyl)oxy)methyl)octahydropyrrolo[1,2-a]pyrazine (2.00 g, 4.4 mmol) and triethylamine (1.35 g, 13.3 mmol) in dichloromethane (15 mL) was added 2,2,2-trifluoroethyl trifluoromethanesulfonate (1.03 g, 4.4 mmol) and stirred at 25° C. for 2 hours. After completion, the reaction mixture was diluted with dichloromethane, washed with water, dried over anhydrous sodium sulfate and concentrated under vacuum. The residue was purified by flash chromatography on silica gel eluting with methanol/dichloromethane (1:30) to afford the title compound (500.0 mg, 1.4 mmol, 32% yield) as a yellow oil. LC-MS: (ESI, m/z): 353.1 [M+H]⁺.

Step 2: ((6R,8aR)-2-(2,2,2-trifluoroethyl)octahydropyrrolo[1,2-a]pyrazin-6-yl)methanol

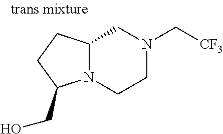 [SMILES: OC[C@H]1CC[C@@H]2CN(CC(F)(F)F)CCN21]

A solution of (6R,8aR)-6-(((tert-butyldimethylsilyl)oxy)methyl)-2-(2,2,2-trifluoroethyl)octahydropyrrolo[1,2-a]pyrazine (500.0 mg, 1.42 mmol) and tetrabutylammonium fluoride (741.7 mg, 2.8 mmol) in tetrahydrofuran (3 mL) was stirred at 50° C. for 8 hours. After completion, the reaction mixture was concentrated under reduced pressure. The residue was purified by flash chromatography on silica gel eluting with methanol/dichloromethane (1:30) to afford the title compound (300.0 mg, 1.3 mmol, 88.8% yield) as a colorless oil. LC-MS: (ESI, m/z): 239.0 [M+H]⁺.

Step 3: 6-((R)-4-((R)-1-(2-(bis(4-methoxybenzyl)amino)pyridin-3-yl)ethyl)-8-chloro-10-fluoro-2-(((6S,8aS)-2-(2,2,2-trifluoroethyl)octahydropyrrolo[1,2-a]pyrazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

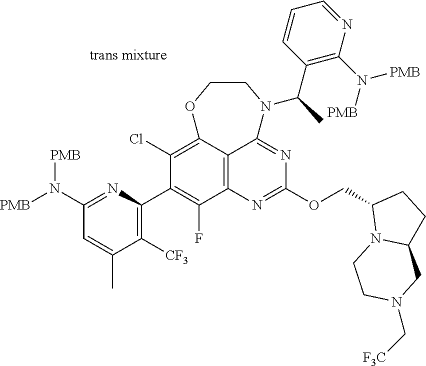

A solution of ((6R,8aR)-2-(2,2,2-trifluoroethyl)octahydropyrrolo[1,2-a]pyrazin-6-yl)methanol (119.9 mg, 0.5 mmol) in tetrahydrofuran (4 mL) was added sodium hydride (50.3 mg, 1.3 mmol, 60% purity) and stirred at 0° C. for 15 min. Then 6-((R)-4-((R)-1-(2-(bis(4-methoxybenzyl)amino)pyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (440.0 mg, 0.4 mmol) was added and stirred at 25° C. for 4 hours. After completion, the reaction was quenched with saturated ammonium chloride solution. The reaction mixture was diluted with ethyl acetate, washed with water, dried over anhydrous sodium sulfate and concentrated under vacuum. The residue was purified by flash chromatography on silica gel eluting with ethyl acetate/petroleum ether (1:8) to afford the title compound (300.0 mg, 0.2 mmol, 57.2% yield) as a white solid. LC-MS: (ESI, m/z): 1250.2 [M+H]⁺.

Step 4: 6-((R)-4-((R)-1-(2-aminopyridin-3-yl)ethyl)-8-chloro-10-fluoro-2-(((6S,8aS)-2-(2,22-trifluoroethyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((R)-1-(2-aminopyridin-3-yl)ethyl)-8-chloro-10-fluoro-2-(((6R,8aR)-2-(2,2,2-trifluoroethyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine

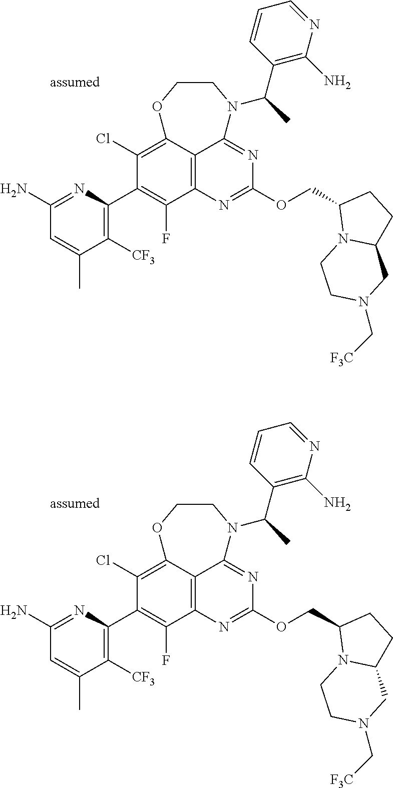

A solution of 6-((R)-4-((R)-1-(2-(bis(4-methoxybenzyl)amino)pyridin-3-yl)ethyl)-8-chloro-10-fluoro-2-(((6S,8aS)-2-(2,2,2-trifluoroethyl)octahydropyrrolo[1,2-a]pyrazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (150.0 mg, 0.1 mmol) in trifluoroacetic acid (2 mL) and trifluoromethanesulfonic acid (0.2 mL) was stirred at 25° C. for 1 hour. After completion, the reaction mixture was purified by flash chromatography on reversed-phase C18 column acetonitrile/water (60:40) to afford crude solid. The crude product was purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 17% B to 42% B in 9 min, 42% B; Wave Length: 254/220 nm) to afford 80 mg product. The product was separated by Chiral-Prep-HPLC with the following conditions (Column: XBridge Prep OBD C18 Column, 30*150 mm, 5 μm; Mobile Phase A: Water (10 mmol/L NH4HCO3), Mobile Phase B: ACN: Flow rate: 60 mL/min: Gradient: 17% B to 42% B in 9 min, 42% B: Wave Length: 254/220 nm) to afford 6-((R)-4-((R)-1-(2-aminopyridin-3-yl)ethyl)-8-chloro-10-fluoro-2-(((6S,8aS)-2-(2,2,2-trifluoroethyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine (18.3 mg, 0.02 mmol, 18.3% yield) and 6-((R)-4-((R)-1-(2-aminopyridin-3-yl)ethyl)-8-chloro-10-fluoro-2-(((6R,8aR)-2-(2,2,2-trifluoroethyl)octahydropyrrolo[1,2-a]pyrazin-6-yl)methoxy)-5,6-dihydro-4H-[1,4]oxazepino[5,6,7-de]quinazolin-9-yl)-4-methyl-5-(trifluoromethyl)pyridin-2-amine (14.1 mg, 0.02 mmol, 14.1% yield).

Example 129a: ¹H NMR (300 MHz, DMSO-d₆, ppm) δ 7.97 (dd, J=4.9, 1.7 Hz, 1H), 7.63 (d, J=7.5 Hz, 1H), 6.80 (s, 2H), 6.66 (dd, J=7.5, 4.9 Hz, 1H), 6.47 (s, 1H), 6.23 (q, J=6.7 Hz, 1H), 5.68 (s, 2H), 4.54-4.32 (m, 2H), 4.26 (dd, J=12.0, 6.7 Hz, 1H), 4.13 (dd, J=10.8, 6.0 Hz, 1H), 3.64 (dd, J=15.5, 6.9 Hz, 1H), 3.56-3.43 (m, 1H), 3.43-3.35 (m, 1H), 3.11 (q, J=10.2 Hz, 2H), 3.05-2.91 (m, 2H), 2.92-2.77 (m, 1H), 2.75-2.64 (m, 1H), 2.63-2.52 (m, 1H), 2.47-2.23 (m, 4H), 2.18-1.95 (m, 2H), 1.92-1.73 (m, 1H), 1.71-1.49 (m, 4H), 1.44-1.26 (m, 1H). LC-MS: (ESI, m/z): 770.4 [M+H]⁺. Chiral HPLC: Column: CHIRALPAK IF-3, 4.6*50 mm, 3 um; Mobile Phase: (Hex:DCM=3:1)(0.1% DEA):EtOH=90:10; Flow: 1.0 mL/min; Retention time: 2.185 min (First peak).

Example 129b: ¹H NMR (300 MHz, DMSO-d₆, ppm) δ 7.97 (dd, J=4.9, 1.7 Hz, 1H), 7.63 (d, J=7.5 Hz, 1H), 6.80 (s, 2H), 6.66 (dd, J=7.5, 4.9 Hz, 1H), 6.47 (s, 1H), 6.23 (q, J=6.7 Hz, 1H), 5.68 (s, 2H), 4.54-4.32 (m, 2H), 4.26 (dd, J=12.0, 6.7 Hz, 1H), 4.13 (dd, J=10.8, 6.0 Hz, 1H), 3.64 (dd, J=15.5, 6.9 Hz, 1H), 3.56-3.43 (m, 1H), 3.43-3.35 (m, 1H), 3.11 (q, J=10.2 Hz, 2H), 3.05-2.91 (m, 2H), 2.92-2.77 (m, 1H), 2.75-2.64 (m, 1H), 2.63-2.52 (m, 1H), 2.47-2.23 (m, 4H), 2.18-1.95 (m, 2H), 1.92-1.73 (m, 1H), 1.71-1.49 (m, 4H), 1.44-1.26 (m, 1H). LC-MS: (ESI, m/z): 770.4 [M+H]⁺. Chiral HPLC: Column: CHIRALPAK IF-3, 4.6*50 mm, 3 um; Mobile Phase: (Hex:DCM=3:1)(0.1% DEA):EtOH=90:10; Flow: 1.0 mL/min; Retention time: 3.048 min (Second peak).

Example 130a & 130b & 130c & 130d: 6-((R)-4-((R)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((S)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((R)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((S)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

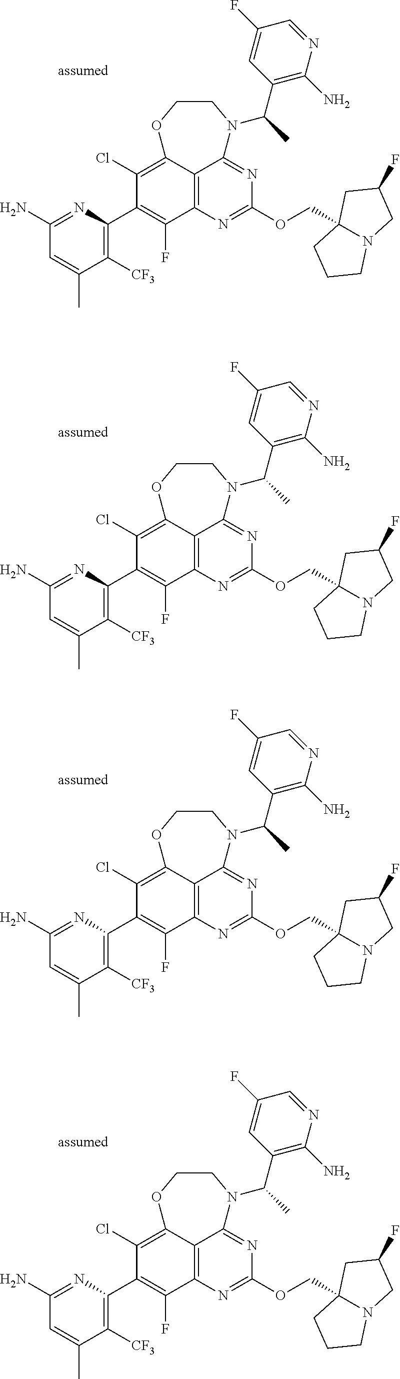

### Synthetic Route

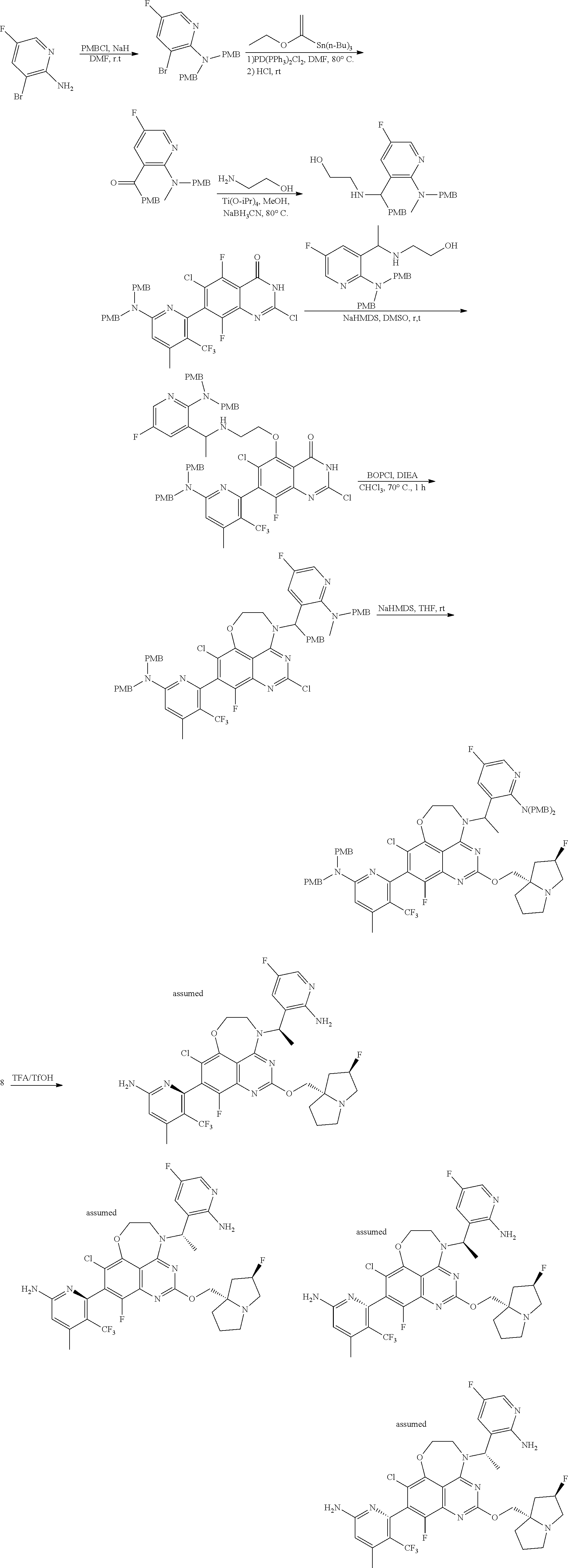

Step 1: 3-bromo-5-fluoro-N,N-bis[(4-methoxyphenyl)methyl]pyridin-2-amine

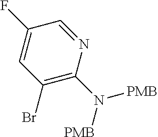

A solution of 3-bromo-5-fluoro-pyridin-2-amine (8.00 g, 41.88 mmol) and 4-methoxybenzylchloride (26.3 g, 167.54 mmol) in N,N-dimethylacetamide (80 mL) was added sodium hydride (5.9 g, 146.6 mmol, 60% purity) and stirred at 25° C. for 1 hour. After completion, the reaction was quenched with saturated ammonium chloride solution, diluted with water, extracted with ethyl acetate, washed with saturated brine, dried over anhydrous sodium sulfate and concentrated under vacuum. The residue was purified by flash chromatography on silica gel eluting with ethyl acetate/petroleum ether (25:1) to afford the title compound (14.3 g, 28.17 mmol, 67.3% yield) as a yellow oil. LC-MS: (ESI, m/z): 431.3 [M+H]⁺

Step 2:1-(2-(bis(4-methoxybenzyl)amino)-5-fluoropyridin-3-yl)ethan-1-one

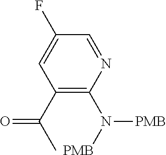

Under nitrogen, a solution of 3-bromo-5-fluoro-N,N-bis[(4-methoxyphenyl)methyl]pyridin-2-amine (13.0 g, 30.14 mmol), bis(triphenylphosphine)palladium(II) chloride (2.1 g, 3.01 mmol) and tributyl(1-ethoxyvinyl)tin (31.0 mL, 90.42 mmol) in N,N-dimethylformamide (100.0 mL) was stirred for 2 h at 80° C. The organic layer was washed with water. The residue was purified by flash chromatography on silica gel eluting with dichloromethane/methanol (9/1) to afford 1-[2-[bis[(4-methoxyphenyl)methyl]amino]-5-fluoro-3-pyridyl]ethanone (12.5 g, 24.16 mmol, 80.2% yield) as a yellow oil. LC-MS: (ESI, m/z): 395.4 [M+H]⁺

Step 2-[1-[2-[bis[(4-methoxyphenyl)methyl]amino]-5-fluoro-3-pyridyl]ethylamino]ethanol

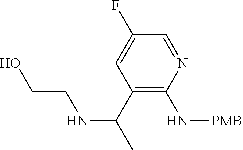

A solution of 1-[5-fluoro-2-[(4-methoxyphenyl)methylamino]-3-pyridyl]ethanone (12.0 g, 30.44 mmol), 2-aminoethanol (6 mL, 91.33 mmol) and titanium tetraisopropanolate (17.3 g, 60.89 mmol) in methyl alcohol (120 mL) was stirred at 80° C. for 2 hours. Then the reaction was cooled to room temperature. And the sodium borohydride (4.6 g, 121.77 mmol) was added and stirred at room temperature. After completion, the solution was diluted with water. Then dichloromethane and diatomite was added into the solution in sequence. The mixture was filtered. The filter was concentrated under vacuum. The residue was purified by flash chromatography on silica gel eluting with dichloromethane/ethyl acetate (1:4) to afford the title compound (1.80 g, 3.69 mmol, 8.4% yield) as a yellow oil. LC-MS: (ESI, m/z): 440.5 [M+H]⁺

Step 4: 7-(6-(bis(4-methoxybenzyl)amino)-4-methyl-3-(trifluoromethyl)pyridin-2-yl)-5-(2-((1-(2-(bis(4-methoxybenzyl)amino)-5-fluoropyridin-3-yl)ethyl)amino)ethoxy)-2,6-dichloro-8-fluoroquinazolin-4(3H)-one

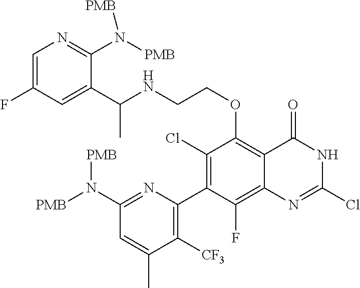

Similar to as described in General Procedure A. Under nitrogen, a solution of 2-[1-[2-[bis[(4-methoxyphenyl)methyl]amino]-5-fluoro-3-pyridyl]ethylamino]ethanol (1.80 g, 4.1 mmol) in dimethyl sulfoxide (10.0 mL) was added sodium bis(trimethylsilyl)amide (15.0 mL, 15 mmol, 1M in tetrahydrofuran) and stirred at 25° C. for 15 minutes. Then 7-[6-[bis[(4-methoxyphenyl)methyl]amino]-4-methyl-3-(trifluoromethyl)-2-pyridyl]-2,6-dichloro-5,8-difluoro-3H-quinazolin-4-one (2.5 g, 3.76 mmol) in dimethyl sulfoxide (10.0 mL) was added and stirred at 60° C. for 30 min. After completion, the reaction was quenched with saturated ammonium chloride solution, diluted with water, extracted with dichloromethane, washed with saturated brine, dried over anhydrous sodium sulfate and concentrated under vacuum to afford the title compound (3.9 g, crude) as a red solid. LC-MS: (ESI, m/z): 1084.3 [M+H]⁺

Step 5: 6-(4-(1-(2-(bis(4-methoxybenzyl)amino)-5-fluoropyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

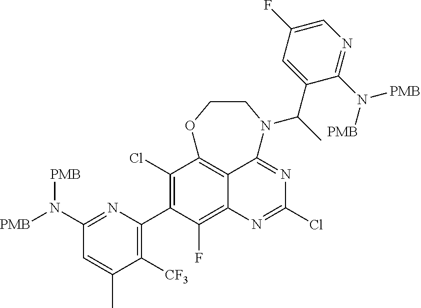

Similar to as described in General Procedure B. A solution of 7-(6-(bis(4-methoxybenzyl)amino)-4-methyl-3-(trifluoromethyl)pyridin-2-yl)-5-(2-((1-(2-(bis(4-methoxybenzyl)amino)-5-fluoropyridin-3-yl)ethyl)amino)ethoxy)-2,6-dichloro-8-fluoroquinazolin-4(3H)-one (4.00 g, crude), N,N-diisopropylethylamine (1.90 g, 14.56 mmol) and bis(2-oxo-3-oxazolidinyl)phosphinic chloride (1.40 g, 5.46 mmol) in chloroform (40 mL) was stirred at 70° C. for 1 hour. After completion, the reaction solution was diluted with ethyl acetate, washed with water, dried over anhydrous sodium sulfate and concentrated under vacuum. The residue was purified by flash chromatography on silica gel eluting with dichloromethane/ethyl acetate (9:1) to afford the title compound (1.20 g, 1.08 mmol, 29.7% yield) as a yellow solid. LC-MS: (ESI, m/z): 1066.3 [M+H]⁺

Step 6: 6-(4-(1-(2-(bis(4-methoxybenzyl)amino)-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

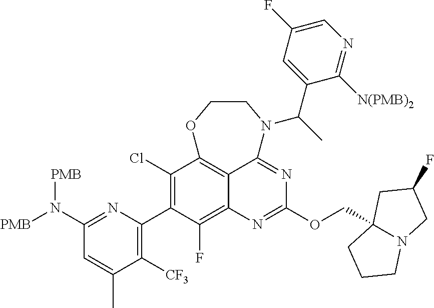

Under nitrogen, a solution of ((2R,7aS)-2-fluorotetrahydro-1H-pyrrolizin-7a(5H)-yl)methanol (365.6 mg, 2.3 mmol) in tetrahydrofuran (5.0 mL) was added sodium bis(trimethylsilyl)amide (3.0 mL, 3.0 mmol, 1M in tetrahydrofuran) and stirred at 25° C. for 15 minutes. Then a solution of 6-(4-(1-(2-(bis(4-methoxybenzyl)amino)-5-fluoropyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (700.0 mg, 0.66 mmol) in tetrahydrofuran (5.0 mL) was added and stirred at 25° C. for 4 hours. After completion, the reaction solution was quenched with saturated ammonium chloride solution and diluted with ethyl acetate, washed with water, dried over anhydrous sodium sulfate and concentrated under vacuum to afford the title compound (435.0 mg, 0.37 mmol, 55.7% yield) as a yellow solid. LC-MS: (ESI, m/z): 1189.5 [M+H]⁺

Step 7: 6-((R)-4-((R)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((S)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((R)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine & 6-((S)-4-((S)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

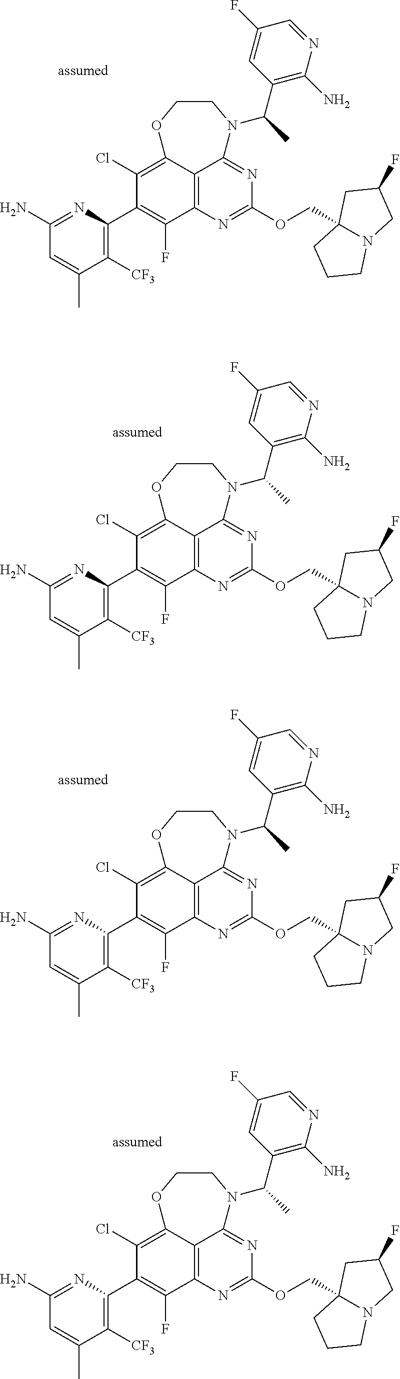

A solution of 6-(4-(1-(2-(bis(4-methoxybenzyl)amino)-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.0 mg, 0.34 mmol) in trifluoroacetic acid (3 mL) and trifluoromethanesulfonic acid (0.3 mL) was stirred at 25° C. for 1 hour. After completion, the reaction solution was concentrated under vacuum, diluted with dichloromethane, adjusted PH=7.0 with saturated sodium carbonate solution, washed with water. dried over anhydrous sodium sulfate and concentrated under vacuum.

The residue was purified by flash chromatography on silica gel eluting with dichloromethane/methanol (6/1) to afford the product. The product was purified by Prep-HPLC with the condition: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 48% B to 68% B in 10 min, 68% B; Wave Length: 254/220 nm; RT1(min): 7.5; to afford 6-((9R)-4-(1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine and 6-((9S)-4-(1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine. The 6-((9R)-4-(1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine was separated by Chiral-Prep-HPLC with condition: Column: CHIRAL ART Cellulose-SC, 2*25 cm, 5 μm; Mobile Phase A: Hex:DCM=3:1(0.5% 2M NH3-MeOH)-HPLC, Mobile Phase B: EtOH-HPLC; Flow rate: 20 mL/min; Gradient: 20% B to 20% B in 9 min; Wave Length: 220/254 nm; RT1(min): 4.52; RT2(min): 7.15; Sample Solvent: EtOH-HPLC; Injection Volume: 1.5 mL; Number Of Runs: 7 to afforded 6-((R)-4-((R)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (5.6 mg, 0.01 mmol, 2.3% yield) and 6-((R)-4-((S)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (4.2 mg, 0.0075 mmol, 1.7% yield).

The 6-((9S)-4-(1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine was was separated by Chiral-Prep-HPLC with condition: Column: CHIRAL ART Cellulose-SC, 2*25 cm, 5 μm; Mobile Phase A: Hex:DCM=3:1(0.5% 2M NH3-MeOH)-HPLC, Mobile Phase B: EtOH-HPLC; Flow rate: 20 mL/min; Gradient: 30% B to 30% B in 10 min; Wave Length: 220/254 nm; RT1(min): 4.32; RT2(min): 7.71; Sample Solvent: EtOH-HPLC; Injection Volume: 1.3 mL; Number Of Runs: 2 to afford 6-((S)-4-((R)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (4.0 mg, 0.0073 mmol, 1.6% yield) and 6-((S)-4-((S)-1-(2-amino-5-fluoropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (9.9 mg, 0.18 mmol, 4.1% yield).

Example 130a: ¹H NMR (300 MHz, Methanol-da, ppm) δ 7.88 (d, J=2.7 Hz, 1H), 7.63 (dd, J=9.1, 2.9 Hz, 1H), 6.59 (s, 1H), 6.49 (q, J=6.9 Hz, 1H), 5.30 (d, J=54.2 Hz, 1H), 4.45 (dd, J=12.8, 5.6 Hz, 1H), 4.38-4.30 (m, 1H), 4.27 (s, 2H), 3.70 (dd, J=15.6, 7.1 Hz, 1H), 3.52 (dd, J=15.8, 5.8 Hz, 1H), 3.27-3.12 (m, 2H), 3.11-2.92 (m, 1H), 2.44 (d, J=2.1 Hz, 3H), 2.41-2.31 (m, 1H), 2.29-2.09 (m, 3H), 2.06-1.82 (m, 3H), 1.66 (d, J=6.8 Hz, 3H). LC-MS: (ESI, m/z): 709.2 [M+H]⁺ Chiral HPLC: Column: CHIRALPAK IC-3, 4.6*50 mm, 3 um; Mobile Phase: (Hex:DCM=3:1)(0.1% DEA):EtOH=70:30; Flow: 1.0 mL/min; Retention time: 1.019 min(First peak).

Example 130b: ¹H NMR (300 MHz, Methanol-d₄, ppm) δ 7.88 (d, J=2.7 Hz, 1H), 7.63 (dd, J=9.1, 2.9 Hz, 1H), 6.59 (s, 1H), 6.49 (q, J=6.9 Hz, 1H), 5.30 (d, J=54.2 Hz, 1H), 4.45 (dd, J=12.8, 5.6 Hz, 1H), 4.37-4.19 (m, 3H), 3.70 (dd, J=15.6, 7.1 Hz, 1H), 3.52 (dd, J=15.8, 5.8 Hz, 1H), 3.27-3.12 (m, 2H), 3.11-2.92 (m, 1H), 2.44 (d, J=2.1 Hz, 3H), 2.41-2.31 (m, 1H), 2.29-2.09 (m, 3H), 2.06-1.82 (m, 3H), 1.66 (d, J=6.8 Hz, 3H). LC-MS: (ESI, m/z): 709.2 [M+H]⁺

Chiral HPLC: Column: CHIRALPAK IC-3, 4.6*50 mm, 3 um; Mobile Phase: (Hex:DCM=3:1)(0.1% DEA):EtOH=70:30; Flow: 1.0 mL/min; Retention time: 1.581 min (Second peak).

Example 130c: ¹H NMR (300 MHz, Methanol-da, ppm) δ 7.88 (d, J=2.7 Hz, 1H), 7.63 (dd, J=9.1, 2.9 Hz, 1H), 6.59 (s, 1H), 6.49 (q, J=6.9 Hz, 1H), 5.30 (d, J=54.2 Hz, 1H), 4.45 (dd, J=12.8, 5.6 Hz, 1H), 4.37-4.19 (m, 3H), 3.70 (dd, J=15.6, 7.1 Hz, 1H), 3.52 (dd, J=15.8, 5.8 Hz, 1H), 3.27-3.12 (m, 2H), 3.11-2.92 (m, 1H), 2.44 (d, J=2.1 Hz, 3H), 2.41-2.31 (m, 1H), 2.29-2.09 (m, 3H), 2.06-1.82 (m, 3H), 1.66 (d, J=6.8 Hz, 3H). LC-MS: (ESI, m/z): 709.2 [M+H]⁺. Chiral HPLC: CHIRALPAK IC-3, 4.6*50 mm, 3 um; Mobile Phase: (Hex:DCM=3:1)(0.1% DEA):EtOH=70:30; Flow: 1.0 mL/min: Retention time: 1.093 min (First peak).

Example 130d: ¹H NMR (300 MHz, Methanol-d₄, ppm) δ 7.88 (d, J=2.7 Hz, 1H), 7.63 (dd, J=9.1, 2.9 Hz, 1H), 6.59 (s, 1H), 6.49 (q, J=6.9 Hz, 1H), 5.30 (d, J=54.2 Hz, 1H), 4.45 (dd, J=12.8, 5.6 Hz, 1H), 4.38-4.30 (m, 1H), 4.27 (s, 2H), 3.70 (dd, J=15.6, 7.1 Hz, 1H), 3.52 (dd, J=15.8, 5.8 Hz, 1H), 3.27-3.12 (m, 2H), 3.11-2.92 (m, 1H), 2.44 (d, J=2.1 Hz, 3H), 2.41-2.31 (m, 1H), 2.29-2.09 (m, 3H), 2.06-1.82 (m, 3H), 1.66 (d, J=6.8 Hz, 3H). LC-MS: (ESI, m/z): 709.2 [M+H]⁺. Chiral HPLC: CHIRALPAK IC-3, 4.6*50 mm, 3 um; Mobile Phase: (Hex:DCM=3:1)(0.1% DEA):EtOH=70:30: Flow: 1.0 mL/min; Retention time: 2.390 (Second peak).

Example 131: 6-(4-((R)-1-(2-amino-5-chloropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

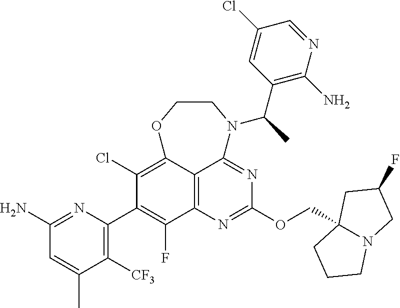 [SMILES: Cc1cc(N)nc(-c2c(Cl)c3c4c(nc(OC[C@@]56CCCN5C[C@H](F)C6)nc4c2F)N([C@H](C)c2cc(Cl)cnc2N)CCO3)c1C(F)(F)F]

### Synthetic Route:

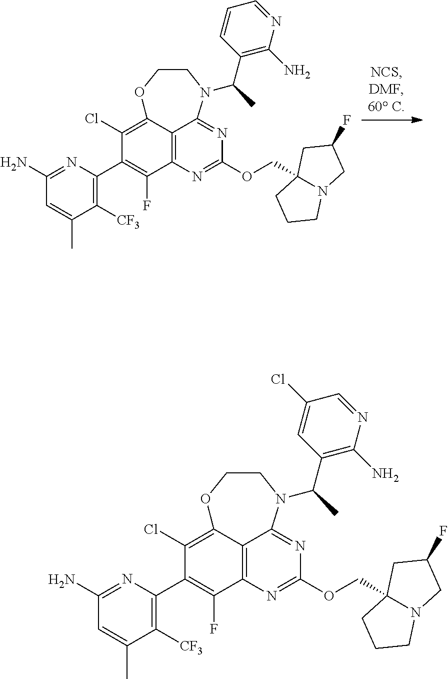 [SMILES: Cc1cc(N)nc(-c2c(Cl)c3c4c(nc(OC[C@@]56CCCN5C[C@H](F)C6)nc4c2F)N([C@H](C)c2cccnc2N)CCO3)c1C(F)(F)F ; Cc1cc(N)nc(-c2c(Cl)c3c4c(nc(OC[C@@]56CCCN5C[C@H](F)C6)nc4c2F)N([C@H](C)c2cc(Cl)cnc2N)CCO3)c1C(F)(F)F]

Step 1: 6-(4-((R)-1-(2-amino-5-chloropyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

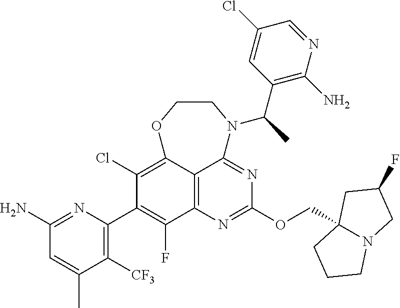 [SMILES: Cc1cc(N)nc(-c2c(Cl)c3c4c(nc(OC[C@@]56CCCN5C[C@H](F)C6)nc4c2F)N([C@H](C)c2cc(Cl)cnc2N)CCO3)c1C(F)(F)F]

A solution of 6-(4-((R)-1-(2-aminopyridin-3-yl)ethyl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (330.0 mg, 0.4800 mmol) and N-chlorosuccinimide (76.0 mg, 0.5700 mmol) in N,N-dimethylformamide (2 mL) was stirred at 60° C. for 1 hour. After completion, the resulting solution was diluted with water and extracted with ethyl acetate. Then the organic layers were washed with brine and concentrated under vacuum. The residue was purified by Prep-HPLC with the following conditions: Column: Xselect CSH C18 OBD Column 30*150 mm 5 μm, n; Mobile Phase A: Water (0.1% FA), Mobile Phase B: ACN; Flow rate: 60 mL/min; Gradient: 20% B to 42% B in 8 min, 42% B; Wave Length: 254/220 nm; RT1(min): 8 to afford the title compound (23.4 mg, 0.029 mmol, 6.2% yield).

Example 131: ¹H NMR (300 MHz, DMSO-d₆, ppm) δ 7.98 (d, J=2.4 Hz, 1H), 7.67 (d, J=2.5 Hz, 1H), 6.81 (s, 2H), 6.48 (s, 1H), 6.21 (d, J=6.9 Hz, 1H), 6.01 (s, 2H), 5.28 (d, J=54.4 Hz, 1H), 4.42-4.30 (m, 2H), 4.07 (q, J=10.3 Hz, 2H), 3.70 (dd, J=15.6, 6.7 Hz, 1H), 3.52-3.37 (m, 2H), 3.15-2.95 (m, 3H), 2.83-2.81 (m, 1H), 2.36 (d, J=2.2 Hz, 3H), 2.19-2.09 (m, 1H), 2.08-2.02 (m, 2H), 1.80-1.76 (m, 2H), 1.58 (d, J=6.8 Hz, 3H). LC-MS: (ESI, m/z): 725.1 [M+H]⁺

### Synthetic Route

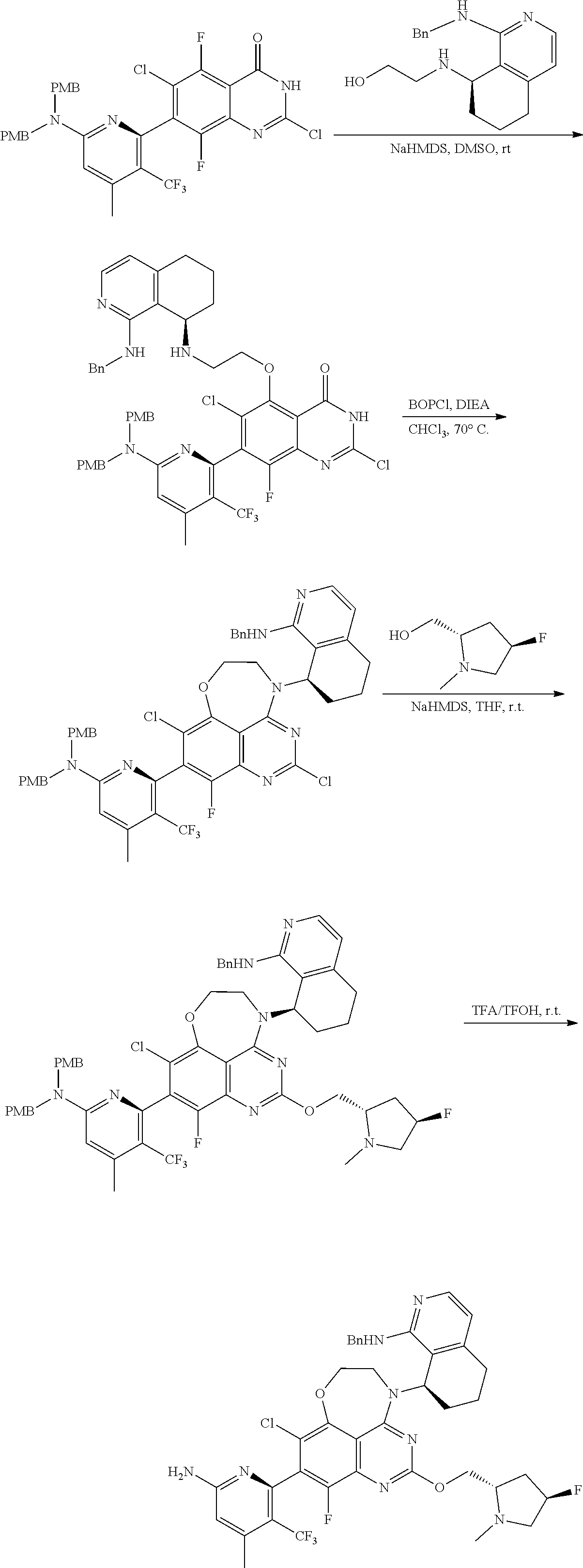

Example 132: (R)-8-((R)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)-5,6,7,8-tetrahydroisoquinolin-1-amine

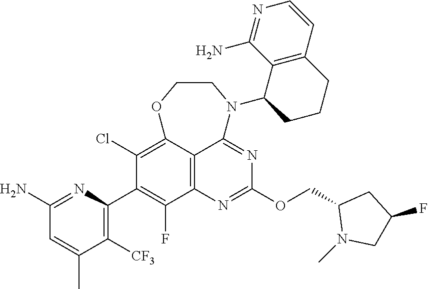

Step 1: 5-(2-(((R)-1-(benzylamino)-5,6,7,8-tetrahydroisoquinolin-8-yl)amino)ethoxy)-7-((R)-6-(bis(4-methoxybenzyl)amino)-4-methyl-3-(trifluoromethyl)pyridin-2-yl)-2,6-dichloro-8-fluoroquinazolin-4(3H)-one

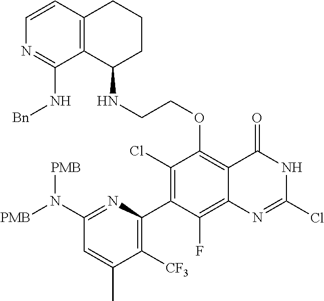

Similar to as described in General Procedure A. To a solution of (R)-2-((1-(benzylamino)-5,6,7,8-tetrahydroisoquinolin-8-yl)amino)ethan-1-ol (223.0 mg, 0.75 mmol) in dimethyl sulfoxide (5 mL) was dropwise added sodium bis(trimethylsilyl)amide (1.13 mL, 1.13 mmol, 1M in tetrahydrofuran) and stirred for 15 min, then (R)-7-(6-(bis(4-methoxybenzyl)amino)-4-methyl-3-(trifluoromethyl)pyridin-2-yl)-2,6-dichloro-5,8-difluoroquinazolin-4(3H)-one (500.0 mg, 0.75 mmol) was added and stirred for 6 hours at room temperature. After completion, the reaction was quenched with saturated ammonium chloride solution. The reaction was diluted with water, extracted with dichloromethane, washed with saturated brine, dried over anhydrous sodium sulfate and concentrated under vacuum. The residue was purified by flash chromatography on silica gel eluting with petroleum ether/ethyl acetate (1:2) to afford the title compound (650.0 mg, 91% yield) as a yellow solid. LC-MS: (ESI, m/z): 942 [M+H]⁺.

Step 2: (R)—N-benzyl-8-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)-5,6,7,8-tetrahydroisoquinolin-1-amine

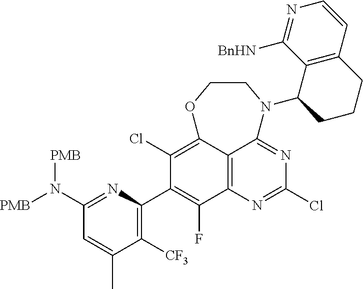

Similar to as described in General Procedure B. To a solution of 5-[2-[[(8R)-1-(benzylamino)-5,6,7,8-tetrahydroisoquinolin-8-yl]amino]ethoxy]-7-[6-[bis[(4-methoxyphenyl)methyl]amino]-4-methyl-3-(trifluoromethyl)-2-pyridyl]-2,6-dichloro-8-fluoro-3H-quinazolin-4-one (200.0 mg, 0.21 mmol), N,N-diisopropylethylamine (136.0 mg, 1.06 mmol) in 1,2-dichloroethane (3 mL) was added bis(2-oxo-3-oxazolidinyl)phosphinic chloride (65.0 mg, 0.26 mmol) and stirred for 2 hours at 70° C. After completion, the reaction was quenched with water, extracted with ethyl acetate, washed with brine, dried over anhydrous sodium sulfate and concentrated under vacuum. The residue was purified by flash chromatography on silica gel eluting with petroleum ether/ethyl acetate (1:2) to afford the title compound (120.0 mg, 61% yield) as a yellow solid. LC-MS: (ESI, m/z): 924 [M+H]⁺.

Step 3: (R)—N-benzyl-8-((R)-9-(6-(bis(4-methoxybenzyl)amino)-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)-5,6,7,8-tetrahydroisoquinolin-1-amine

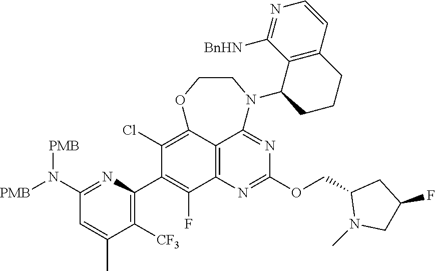

Under nitrogen, a solution of ((2S,4R)-4-fluoro-1-methylpyrrolidin-2-yl)methanol (30.0 mg, 0.23 mmol) in tetrahydrofuran (2 mL) was dropwise added sodium bis(trimethylsilyl)amide (0.16 mL, 0.32 mmol, 2M in tetrahydrofuran) and stirred for 15 minutes at 0° C. Then the reaction solution was transferred into a solution of (R)—N-benzyl-8-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)-5,6,7,8-tetrahydroisoquinolin-1-amine (150 mg, 0.16 mmol) in tetrahydrofuran (5 mL) and stirred for 2 hours at 0° C. After completion, the reaction was quenched with saturated ammonium chloride solution. The reaction was diluted with ethyl acetate, washed with saturated brine, dried over anhydrous sodium sulfate and concentrated under vacuum. The residue was purified by flash chromatography on silica gel eluting with dichloromethane/methanol to afford the title compound (90.0 mg, 54% yield). LC-MS: (ESI, m/z): 1021 [M+H]⁺.

Step 4: (R)-8-((R)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)-5,6,7,8-tetrahydroisoquinolin-1-amine

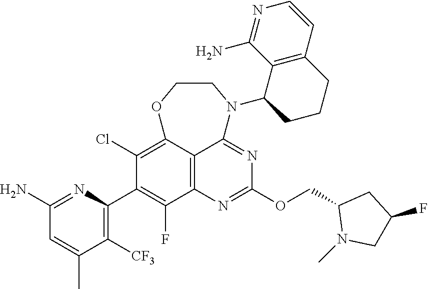

A mixture of (R)—N-benzyl-8-((R)-9-(6-(bis(4-methoxybenzyl)amino)-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)-5,6,7,8-tetrahydroisoquinolin-1-amine (90.0 mg, 0.09 mmol) in trifluoromethanesulfonic acid (0.1 mL) and 2,2,2-trifluoroacetic acid (1 mL) was stirred for 8 hours at 25° C. After completion, the residue was concentrated under vacuum and purified by Prep-HPLC with the following conditions (Column: XSelect CSH Fluoro Phenyl, 30*150 mm, 5 μm; Mobile Phase A: Water (10 mmol/L NH4HCO3), Mobile Phase B: ACN: Flow rate: 60 mL/min: Gradient: 33% B to 58% B in 9 min, 58% B; Wave Length: 220/254 nm) to afford the title compound (13.0 mg, 21.4% yield). LC-MS: (ESI, m/z): 691.2 [M+H]⁺

Example 132: ¹H NMR (300 MHz, Methanol-d₄, ppm) δ 7.81 (d, J=5.3 Hz, 1H), 6.58 (d, J=5.5 Hz, 2H), 6.34 (t, J=6.6 Hz, 1H), 5.18 (d, J=55.4 Hz, 1H), 4.52 (d, J=5.2 Hz, 2H), 4.49-4.36 (m, 2H), 3.80-3.32 (m, 3H), 3.25-3.06 (m, 1H), 2.99-2.59 (m, 3H), 2.55 (s, 3H), 2.44 (dd, J=2.1, 0.9 Hz, 3H), 2.38-2.19 (m, 2H), 2.20-1.53 (m, 4H).

Example 133a & 133b: 4-(1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine & 4-(1-((S)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine

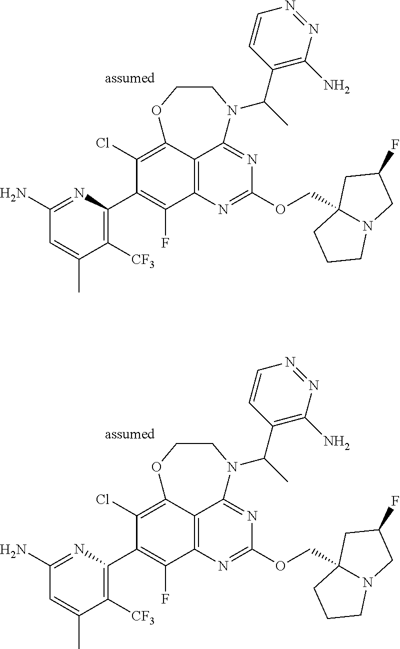

### Synthetic Route

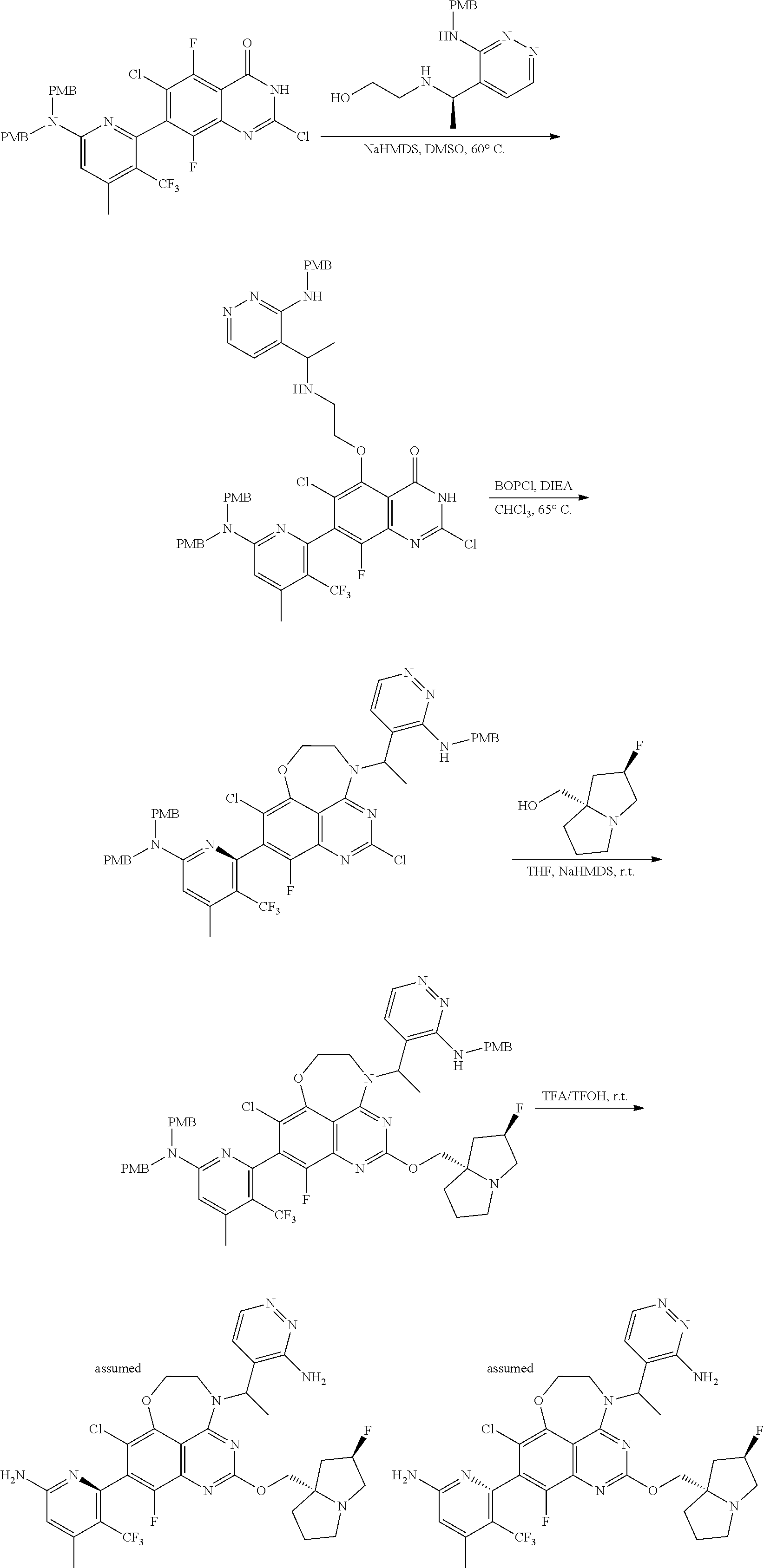

Step 1: 7-(6-(bis(4-methoxybenzyl)amino)-4-methyl-3-(trifluoromethyl)pyridin-2-yl)-2,6-dichloro-8-fluoro-5-(2-((1-(3-((4-methoxybenzyl)amino)pyridazin-4-yl)ethyl)amino)ethoxy)quinazolin-4(3H)-one

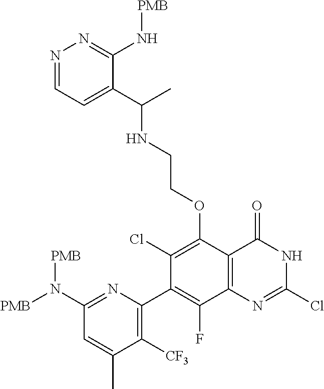

Similar to as described in General Procedure A. A solution of (R)-2-((1-(3-((4-methoxybenzyl)amino)pyridazin-4-yl)ethyl)amino)ethan-1-ol (3.01 g, 9.96 mmol) in dimethyl sulfoxide (70 mL) was added sodium bis(trimethylsilyl)amide[1.0 M in tetrahydrofuran](22.9 mL, 22.9 mmol) and stirred at 25° C. for 30 minutes. Then 7-(6-(bis(4-methoxybenzyl)amino)-4-methyl-3-(trifluoromethyl)pyridin-2-yl)-2,6-dichloro-5,8-difluoroquinazolin-4(3H)-one (5.10 g, 7.66 mmol) was added and stirred at 60° C. for 2 hours. After completion, the reaction was quenched by ammonium chloride aqueous solution. The reaction mixture was diluted with ethyl acetate. The reaction solution was washed with water, dried over anhydrous sodium sulfate and concentrated under vacuum to afford the title compound (9.40 g, crude) as a yellow solid. LC-MS: (ESI, m/z): 947.3 [M+H]⁺

Step 2: 4-(1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine

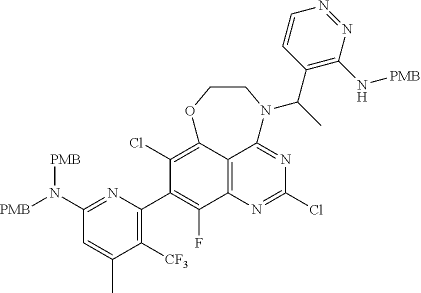

Similar to as described in General Procedure B. A solution of 7-(6-(bis(4-methoxybenzyl)amino)-4-methyl-3-(trifluoromethyl)pyridin-2-yl)-2,6-dichloro-8-fluoro-5-(2-((1-(3-((4-methoxybenzyl)amino)pyridazin-4-yl)ethyl)amino)ethoxy)quinazolin-4(3H)-one (8.40 g, 8.86 mmol) and N,N-diisopropylethylamine (3.43 g, 26.59 mmol) in chloroform (100 mL) was added bis(2-oxo-3-oxazolidinyl)phosphinic chloride (4.05 g, 15.95 mmol) and stirred at 65° C. for 1 hour. After completion, the reaction mixture was diluted with dichloromethane. The organic layer was washed with water, dried over anhydrous sodium sulfate and concentrated under vacuum. The residue was purified by flash chromatography on silica gel eluting with ethyl acetate/dichloromethane (1:5) to afford the title compound (4.00 g, 4.13 mmol, 46.6% yield) as a yellow solid. LC-MS: (ESI, m/z): 929.3 [M+H]⁺

Step 3: 4-(1-(9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyridazin-3-amine

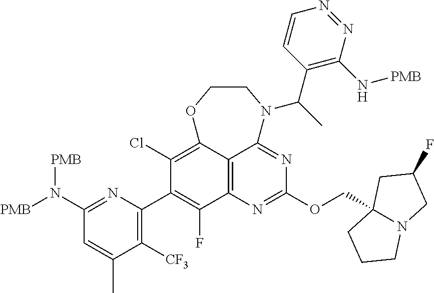

A solution of ((2R,7aS)-2-fluorotetrahydro-1H-pyrrolizin-7a(5H)-yl)methanol (924.6 mg, 5.8 mmol) in tetrahydrofuran (26 mL) was added sodium bis(trimethylsilyl)amide[1.0 M in tetrahydrofuran] (6.7 mL, 6.7 mmol) and stirred at 25° C. for 30 minutes. Then 4-(1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine (1.80 g, 1.94 mmol) was added and stirred at 25° C. for 2 hours. After completion, the reaction was quenched by ammonium chloride aqueous solution. The reaction mixture was diluted with ethyl acetate. The organic layer was washed with water, dried over anhydrous sodium sulfate and concentrated under vacuum. The residue was purified by flash chromatography on silica gel eluting with ethyl acetate/dichloromethane (3:1) to afford the title compound (2.50 g, 1.52 mmol, 78.5% yield) as a yellow solid. LC-MS: (ESI, m/z): 1052.4 [M+H]⁺

Step 4: 4-(1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine & 4-(1-((S)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine

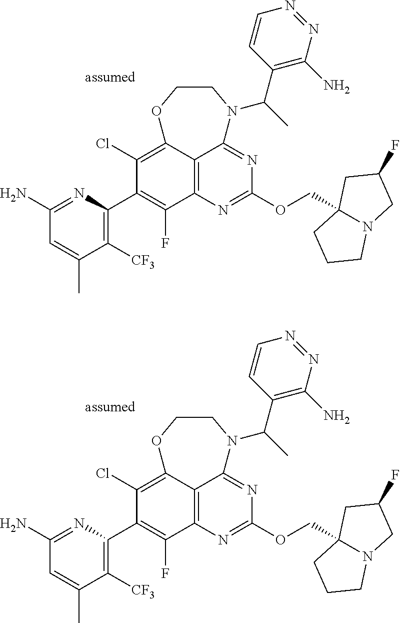

A solution of 4-(1-(9-(6-(bis(4-methoxybenzyl)amino)-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyridazin-3-amine (2.30 g, 1.40 mmol) in trifluoromethanesulfonic acid (2 mL) and 2,2,2-trifluoroacetic acid (20 mL) was stirred at 25° C. for 1 hour. After completion, the solvent was concentrated under vacuum. The reaction mixture was diluted with ethyl acetate. The reaction mixture was adjusted to pH=8 with aqueous sodium carbonate solution. The organic layer was washed with water. The organic layer was dried over anhydrous sodium sulfate and concentrated under vacuum. The crude product was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min: Gradient: 27% B to 57% B in 7 min; Wave Length: 254 nm; RT1(min): 6.5, to afford 4-(1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine (273.8 mg, 0.3 mmol, 28.2% yield) and 4-(1-((S)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine (255.8 mg, 0.3 mmol, 27.0% yield) as a yellow solid. The racemization of benzylic methyl was observed so the diastereomers were purified and separated by Prep SFC with the following conditions (Column: Chiralpak IJ column, 30*150 mm, 5 μm; Mobile Phase A: Carbon Dioxide, Mobile Phase B: Methanol w/0.1% Ammonium Hydroxide; Isocratic: 25% B 9 min, Cycle time: 8 min; Flow rate: 125 mL/min: Pressure: 100 bar; Temperature: 40° C.: Wave Length: 240 nm; RT1(min): 2.7 min. and RT2 (min): 4.6 min, to afford 4-(1-((R)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine & 4-(1-((S)-9-(6-amino-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyridazin-3-amine (121.5 mg, 0.14 mmol, 12.8% yield) as a single diastereomer.

Example 133a: ¹H NMR (400 MHz, DMSO-d₆, ppm) δ 8.52 (dd, J=4.9, 1.3 Hz, 1H), 7.37 (dd, J=5.0, 2.1 Hz, 1H), 6.82 (s, 2H), 6.48 (s, 1H), 6.27 (s, 2H), 6.10 (q, J=6.9, 1H), 5.27 (d, J=54.0 Hz, 1H), 4.50 (dd, J=12.3, 6.4 Hz, 1H), 4.39 (dd, J=12.5, 6.7 Hz, 1H), 4.13-3.92 (m, 2H), 3.77 (dd, J=15.6, 6.8 Hz, 1H), 3.52 (dd, J=15.7, 6.3 Hz, 1H), 3.13-2.95 (m, 3H), 2.88-2.68 (m, 1H), 2.36 (s, 3H), 2.17-2.05 (m, 1H), 2.05-1.90 (m, 2H), 1.88-1.68 (m, 3H), 1.58 (d, J=6.8 Hz, 3H). LC-MS: (ESI, m/z): 692.2 [M+H]⁺

Example 133b: ¹H NMR (400 MHz, DMSO-d₆, ppm) δ 8.52 (dd, J=4.9, 1.3 Hz, 1H), 7.37 (dd, J=5.0, 2.1 Hz, 1H), 6.82 (s, 2H), 6.48 (s, 1H), 6.27 (s, 2H), 6.10 (q, J=6.9, 1H), 5.27 (d, J=54.0 Hz, 1H), 4.50 (dd, J=12.3, 6.4 Hz, 1H), 4.39 (dd, J=12.5, 6.7 Hz, 1H), 4.13-3.92 (m, 2H), 3.77 (dd, J=15.6, 6.8 Hz, 1H), 3.52 (dd, J=15.7, 6.3 Hz, 1H), 3.13-2.95 (m, 3H), 2.88-2.68 (m, 1H), 2.36 (s, 3H), 2.17-2.05 (m, 1H), 2.05-1.90 (m, 2H), 1.88-1.68 (m, 3H), 1.58 (d, J=6.8 Hz, 3H). LC-MS: (ESI, m/z): 692.2 [M+H]⁺

Example 133c: ¹H NMR (400 MHz, DMSO-d₆, ppm) δ 8.51 (d, J=4.9 Hz, 1H), 7.36 (d, J=4.8 Hz, 1H), 6.79 (s, 2H), 6.47 (s, 1H), 6.24 (s, 2H), 6.09 (q, J=6.8 Hz, 1H), 5.36-5.16 (m, 1H), 4.54-4.33 (m, 2H), 4.11-3.92 (m, 2H), 3.81-3.69 (m, 1H), 3.56-3.47 (m, 1H), 3.14-2.95 (m, 3H), 2.85-2.76 (m, 1H), 2.39-2.30 (m, 3H), 2.14-1.92 (m, 3H), 1.86-1.67 (m, 3H), 1.58 (d, J=6.8 Hz, 3H). LC-MS: (ESI, m/z): 692.2 [M+H]⁺

LCMS analysis condition (for examples 134-140). Experiments were run using Method A: on an Agilent LCMS system equipped with DAD and ELSD detector with an Ion mode Positive. The column used was a Waters X-Bridge C18, 50*2.1 mm*5 μm or equivalent (Mobile Phase: A: water (0.04% trifluoroacetic acid); B: acetonitrile (0.02% trifluoroacetic acid)) using a gradient of 4.5 min gradient method, actual method would depend on clog P of compound and a Flow Rate: 0.6 mL/min or 0.8 mL/min. The Column Temp was 40° C. or 50° C. with detection performed at 220 nm.

Example 134: 6-((9R)-4-((R)-1-(2-aminopyridin-3-yl)ethyl)-8-chloro-10-fluoro-2-((1-methylpyrrolidin-3-yl)methoxy)-5,6-dihydro-4H-[1,4]oxazepino[5,6,7-de]quinazolin-9-yl)-4-methyl-5-(trifluoromethyl)pyridin-2-amine

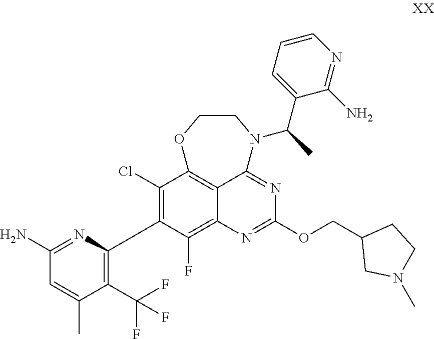

### Synthetic Route

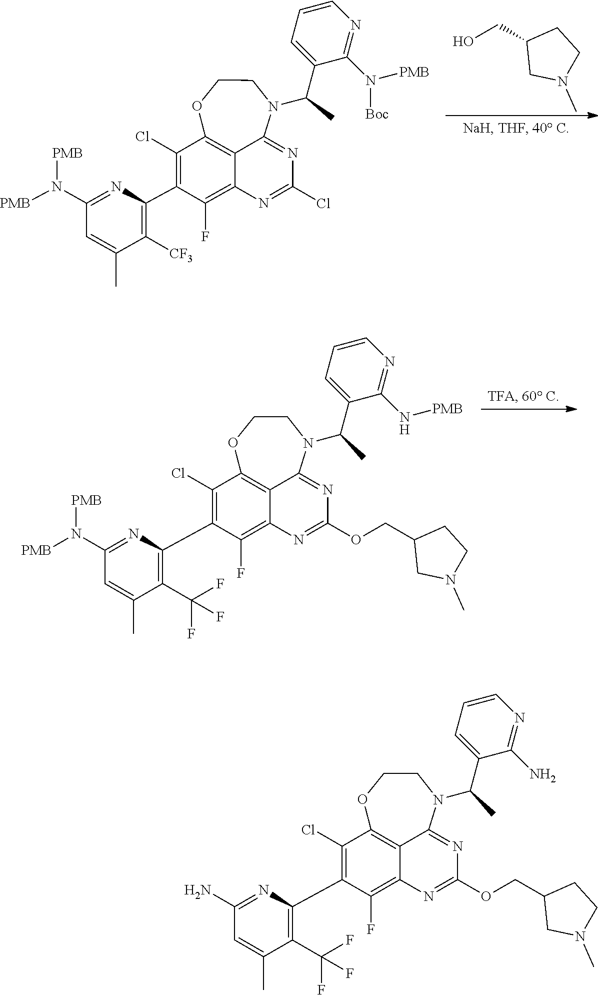

Step 1: tert-butyl (3-((1R)-1-((9R)-9-(6-(bis(4-methoxybenzyl)amino)-4-methyl-3-(trifluoromethyl)pyridin-2-yl)-8-chloro-10-fluoro-2-((1-methylpyrrolidin-3-yl)methoxy)-5,6-dihydro-4H-[1,4]oxazepino[5,6,7-de]quinazolin-4-yl)ethyl)pyridin-2-yl)carbamate

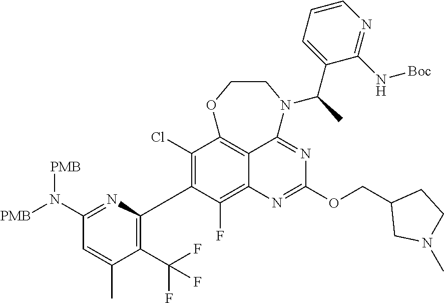

(1-methylpyrrolidin-3-yl)methanol (0.15 mmol) was reacted with Di-tert-butyl (3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (50.4 mg, 0.050 mmol) to give the crude title compound as a tan oil which was carried forward to the next step.

Step 2: 6-((9R)-4-((R)-1-(2-aminopyridin-3-yl)ethyl)-8-chloro-10-fluoro-2-((1-methylpyrrolidin-3-yl)methoxy)-5,6-dihydro-4H-[1,4]oxazepino[5,6,7-de]quinazolin-9-yl)-4-methyl-5-(trifluoromethyl)pyridin-2-amine

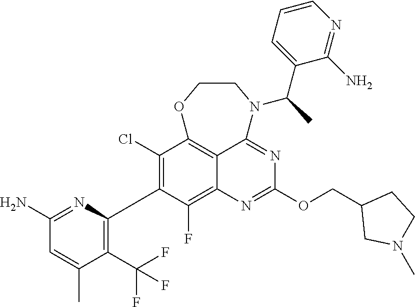

A solution of tert-butyl (3-((1R)-1-((9R)-9-(6-amino-4-methyl-3-(trifluoromethyl)pyridin-2-yl)-8-chloro-10-fluoro-2-((1-methylpyrrolidin-3-yl)methoxy)-5,6-dihydro-4H-[1,4]oxazepino[5,6,7-de]quinazolin-4-yl)ethyl)pyridin-2-yl)carbamate (˜0.03 mmol) in trifluoroacetic acid (1 mL) was stirred for 5 hours at 25° C. After completion, the reaction mixture was concentrated under vacuum. The product was purified by GILSON 281 and Shimadzu LCMS 2010A Prep-HPLC with the following conditions: Column: Xtimate C18 Column, 25*150 mm, 5 μm; Mobile Phase A: formic acid/water (0.225%), Mobile Phase B: acetonitrile; Flow rate: 25 mL/min; Gradient: 30% B to 70% B in 12.5 min; Wave Length: 254/220 nm to afford 6-((9R)-4-((R)-1-(2-aminopyridin-3-yl)ethyl)-8-chloro-10-fluoro-2-((1-methylpyrrolidin-3-yl)methoxy)-5,6-dihydro-4H-[1,4]oxazepino[5,6,7-de]quinazolin-9-yl)-4-methyl-5-(trifluoromethyl)pyridin-2-amine (16.1 mg). LC-MS: (ESI, m/z): 647.3 [M+H]⁺, RT1(min) 1.794

Example 134: ¹H NMR (400 MHz, DMSO-d₆, ppm) δ 7.98 (dd, J=5.0, 1.8 Hz, 1H), 7.64 (d, J=7.4 Hz, 1H), 6.79 (s, 2H), 6.68 (dd, J=7.5, 4.9 Hz, 1H), 6.48 (s, 1H), 6.22 (q, J=7.0 Hz, 1H), 5.74 (s, 2H), 4.50-4.30 (m, 3H), 4.30-4.21 (m, 1H), 3.90-3.48 (m, 3H), 3.45-3.36 (m, 2H), 3.01-2.63 (m, 5H), 2.39-2.33 (m, 3H), 2.29-2.05 (m, 1H), 2.01-1.65 (m, 1H), 1.56 (d, J=6.8 Hz, 3H). LC-MS: (ESI, m/z): 647.3 [M+H]⁺

Example 135: 6-((R)-4-((R)-1-(2-aminopyridin-3-yl)ethyl)-8-chloro-10-fluoro-2-(2-(4-methylpiperazin-1-yl)ethoxy)-5,6-dihydro-4H-[1,4]oxazepino[5,6,7-de]quinazolin-9-yl)-4-methyl-5-(trifluoromethyl)pyridin-2-amine

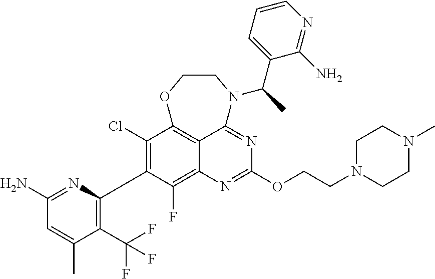

### Synthetic Route

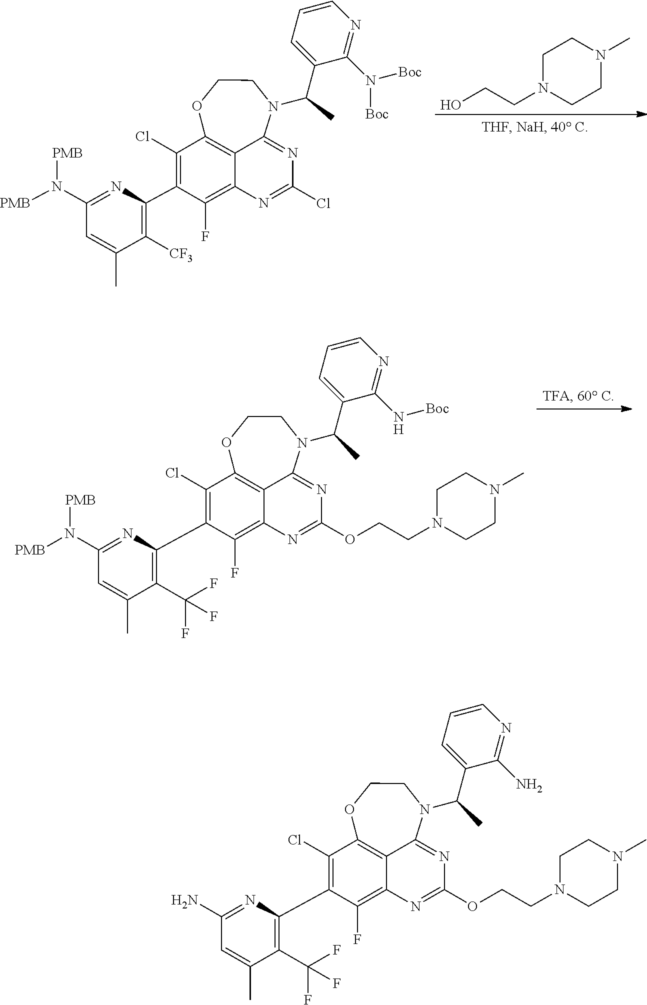

Step 1: tert-butyl (3-((R)-1-((R)-9-(6-(bis(4-methoxybenzyl)amino)-4-methyl-3-(trifluoromethyl)pyridin-2-yl)-8-chloro-10-fluoro-2-(2-(4-methylpiperazin-1-yl)ethoxy)-5,6-dihydro-4H-[1,4]oxazepino[5,6,7-de]quinazolin-4-yl)ethyl)pyridin-2-yl)carbamate

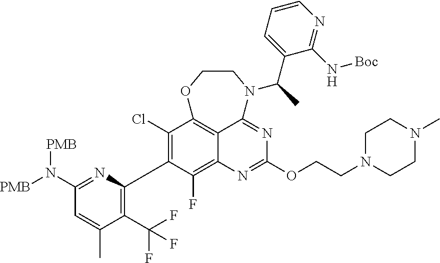

2-(4-methylpiperazin-1-yl)ethan-1-ol (0.15 mmol) was reacted with Di-tert-butyl (3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1, 4]oxazepino[5,6,7-de]quinazolin-4-yl)ethyl)pyridin-2-yl)iminocarboxylate (50.4 mg, 0.050 mmol) to give the crude title compound as a tan oil which was carried forward to the next step.

Step 2: 6-((R)-4-((R)-1-(2-aminopyridin-3-yl)ethyl)-8-chloro-10-fluoro-2-(2-(4-methylpiperazin-1-yl)ethoxy)-5,6-dihydro-4H-[1,4]oxazepino[5,6,7-de]quinazolin-9-yl)-4-methyl-5-(trifluoromethyl)pyridin-2-amine

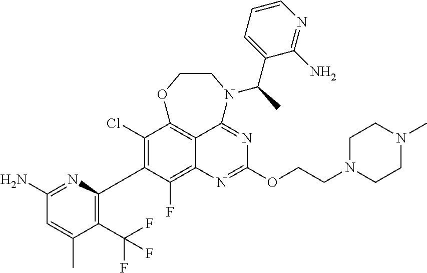

A solution of tert-butyl (3-((R)-1-((R)-9-(6-(bis(4-methoxybenzyl)amino)-4-methyl-3-(trifluoromethyl)pyridin-2-yl)-8-chloro-10-fluoro-2-(2-(4-methylpiperazin-1-yl)ethoxy)-5,6-dihydro-4H-[1,4]oxazepino[5,6,7-de]quinazolin-4-yl)ethyl)pyridin-2-yl)carbamate (˜0.03 mmol) in trifluoroacetic acid (1 mL) was stirred for 5 hours at 25° C. After completion, the reaction mixture was concentrated under vacuum. The product was purified by GILSON 281 and Shimadzu LCMS 2010A Prep-HPLC with the following conditions: Column: Xtimate C18 Column, 25*150 mm, 5 μm; Mobile Phase A: formic acid/water (0.225%), Mobile Phase B: acetonitrile; Flow rate: 25 mL/min; Gradient: 30% B to 70% B in 12.5 min; Wave Length: 254/220 nm to afford 6-((R)-4-((R)-1-(2-aminopyridin-3-yl)ethyl)-8-chloro-10-fluoro-2-(2-(4-methylpiperazin-1-yl)ethoxy)-5,6-dihydro-4H-[1,4]oxazepino[5,6,7-de]quinazolin-9-yl)-4-methyl-5-(trifluoromethyl)pyridin-2-amine (14.1 mg). LC-MS: (ESI, m/z): 676.3 [M+H]⁺, RT1(min) 1.744

Example 135: ¹H NMR (400 MHz, DMSO-d₆) δ 7.97 (dd, J=4.9, 1.8 Hz, 1H), 7.63 (d, J=7.5 Hz, 1H), 6.79 (s, 2H), 6.67 (dd, J=7.5, 4.9 Hz, 1H), 6.47 (s, 1H), 6.24 (q, J=6.8 Hz, 1H), 5.67 (s, 2H), 4.53-4.38 (m, 3H), 4.29-4.21 (m, 1H), 3.69-3.60 (m, 1H), 3.42-3.33 (m, 2H), 3.12-2.71 (m, 6H), 2.67-2.52 (m, 3H), 2.44-2.09 (m, 6H), 1.56 (d, J=6.8 Hz, 3H). LC-MS: (ESI, m/z): 676.3 [M+H]⁺

Example 136: 6-((R)-2-(2-(1H-pyrazol-1-yl)ethoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

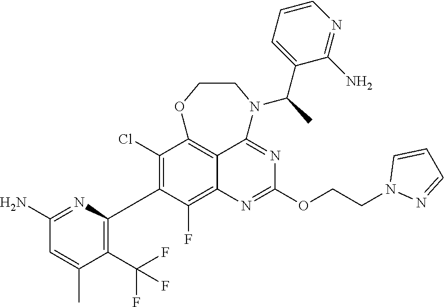

### Synthetic Route

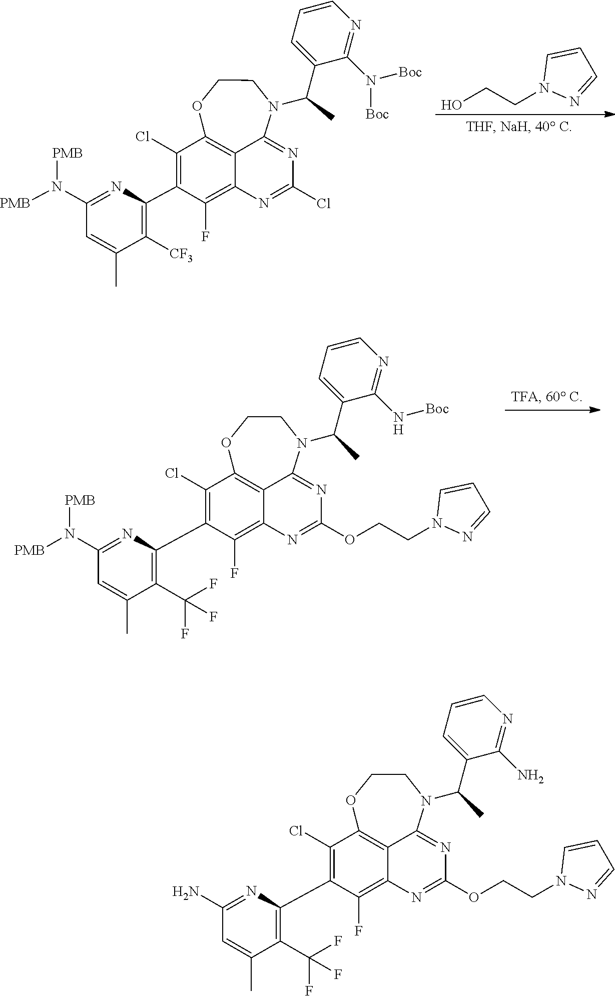

Step 1: tert-butyl (3-((R)-1-((R)-2-(2-(1H-pyrazol-1-yl)ethoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate

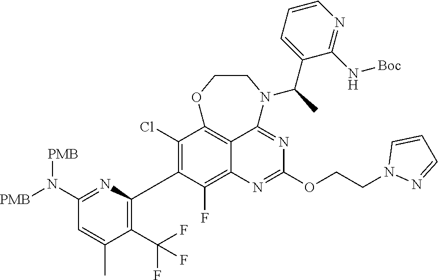

2-(1H-pyrazol-1-yl)ethan-1-ol (0.15 mmol) was reacted with Di-tert-butyl (3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (50.4 mg, 0.050 mmol) to give the crude title compound as a tan oil which was carried forward to the next step.

Step 2: 6-((R)-2-(2-(1H-pyrazol-1-yl)ethoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

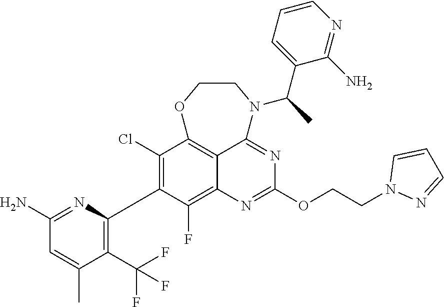

A solution of tert-butyl (3-((R)-1-((R)-2-(2-(1H-pyrazol-1-yl)ethoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate (˜0.03 mmol) in trifluoroacetic acid (1 mL) was stirred for 5 hours at 55° C. After completion, the reaction mixture was concentrated under vacuum. The product was purified by GILSON 281 and Shimadzu LCMS 2010A Prep-HPLC with the following conditions: Column: Xtimate C18 Column, 25*150 mm, 5 μm; Mobile Phase A: formic acid/water (0.225%), Mobile Phase B: acetonitrile; Flow rate: 25 mL/min; Gradient: 30% B to 70% B in 12.5 min; Wave Length: 254/220 nm to afford 6-((R)-2-(2-(1H-pyrazol-1-yl)ethoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (6.1 mg). LC-MS: (ESI, m/z): 644.2 [M+H]⁺, RT1(min) 1.979

Example 136: ¹H NMR (300 MHz, DMSO-d₆, ppm) δ ¹H NMR (400 MHz, DMSO-de, ppm) δ 8.01-7.95 (m, 1H), 7.82-7.67 (m, 2H), 7.48-7.43 (m, 1H), 6.77 (d, J=17.7 Hz, 3H), 6.53-6.44 (m, 2H), 6.25 (t, J=2.1 Hz, 1H), 6.22-6.14 (m, 1H), 4.76-4.67 (m, 2H), 4.55-4.48 (m, 2H), 4.48-4.39 (m, 1H), 4.30 (s, 1H), 3.73-3.61 (m, 1H), 3.45-3.36 (m, 2H), 2.38-2.34 (m, 3H), 1.57 (d, J=6.8 Hz, 3H). LC-MS: (ESI, m/z): 644.2 [M+H]⁺

Example 137: 6-((9R)-4-((R)-1-(2-aminopyridin-3-yl)ethyl)-8-chloro-10-fluoro-2-((1-methylpiperidin-3-yl)methoxy)-5,6-dihydro-4H-[1,4]oxazepino[5,6,7-de]quinazolin-9-yl)-4-methyl-5-(trifluoromethyl)pyridin-2-amine

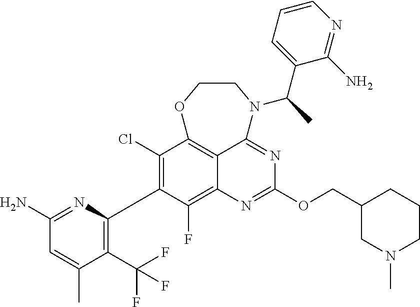

### Synthetic Route

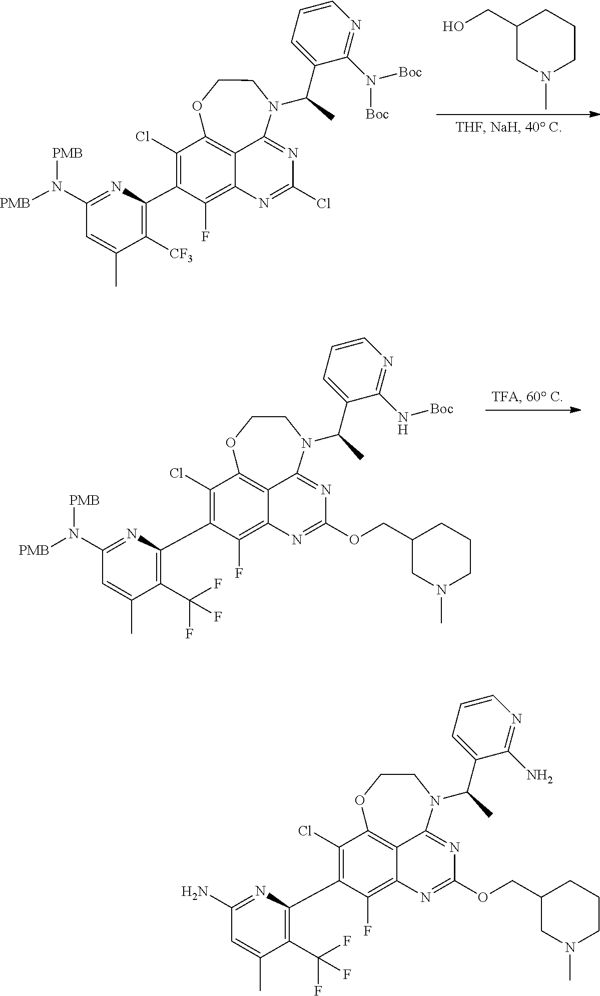

Step 1: tert-butyl (3-((1R)-1-((9R)-9-(6-(bis(4-methoxybenzyl)amino)-4-methyl-3-(trifluoromethyl)pyridin-2-yl)-8-chloro-10-fluoro-2-((1-methylpiperidin-3-yl)methoxy)-5,6-dihydro-4H-[1,4]oxazepino[5,6,7-de]quinazolin-4-yl)ethyl)pyridin-2-yl)carbamate

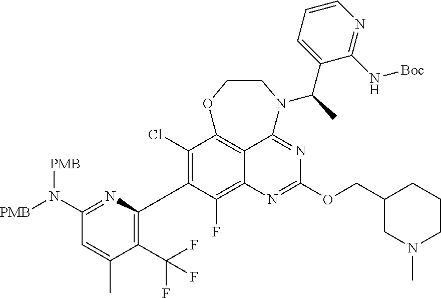

(1-methylpiperidin-3-yl)methanol (0.15 mmol) was reacted with Di-tert-butyl (3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (50.4 mg, 0.050 mmol) to give the crude title compound as a tan oil which was carried forward to the next step.

Step 2: 6-((9R)-4-((R)-1-(2-aminopyridin-3-yl)ethyl)-8-chloro-10-fluoro-2-((1-methylpyrrolidin-3-yl)methoxy)-5,6-dihydro-4H-[1,4]oxazepino[5,6,7-de]quinazolin-9-yl)-4-methyl-5-(trifluoromethyl)pyridin-2-amine

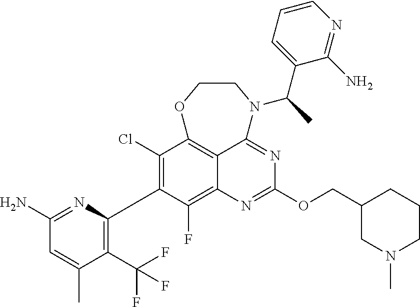

A solution of tert-butyl (3-((1R)-1-((9R)-9-(6-(bis(4-methoxybenzyl)amino)-4-methyl-3-(trifluoromethyl)pyridin-2-yl)-8-chloro-10-fluoro-2-((1-methylpiperidin-3-yl)methoxy)-5,6-dihydro-4H-[1,4]oxazepino[5,6,7-de]quinazolin-4-yl)ethyl)pyridin-2-yl)carbamate (˜0.03 mmol) in trifluoroacetic acid (1 mL) was stirred for 5 hours at 55° C. After completion, the reaction mixture was concentrated under vacuum. The product was purified by GILSON 281 and Shimadzu LCMS 2010A Prep-HPLC with the following conditions: Column: Xtimate C18 Column, 25*150 mm, 5 μm; Mobile Phase A: formic acid/water (0.225%), Mobile Phase B: acetonitrile; Flow rate: 25 mL/min; Gradient: 30% B to 70% B in 12.5 min; Wave Length: 254/220 nm to afford 6-((9R)-4-((R)-1-(2-aminopyridin-3-yl)ethyl)-8-chloro-10-fluoro-2-((1-methylpiperidin-3-yl)methoxy)-5,6-dihydro-4H-[1,4]oxazepino[5,6,7-de]quinazolin-9-yl)-4-methyl-5-(trifluoromethyl)pyridin-2-amine (10.4 mg). LC-MS: (ESI, m/z): 661.3 [M+H]⁺, RT1(min) 2.131

Example 137: ¹H NMR (400 MHz, DMSO-d₆, ppm) δ 7.97 (d, J=4.9 Hz, 1H), 7.63 (d, J=7.7 Hz, 1H), 6.79 (s, 2H), 6.67 (dd, J=7.4, 4.9 Hz, 1H), 6.47 (s, 1H), 6.22 (q, J=6.0, 5.3 Hz, 1H), 5.70 (s, 2H), 4.48-4.40 (m, 1H), 4.40-4.30 (m, 1H), 4.30-4.18 (m, 2H), 3.71-3.62 (m, 1H), 3.58-3.35 (m, 3H), 2.77 (s, 5H), 2.35 (s, 3H), 2.30-2.15 (m, 1H), 1.90-1.61 (m, 3H), 1.57 (d, J=6.8 Hz, 3H), 1.36-1.20 (m, 1H). LC-MS: (ESI, m/z): 661.3 [M+H]⁺

Example 138: 6-((R)-4-((R)-1-(2-aminopyridin-3-yl)ethyl)-8-chloro-10-fluoro-2-(2-morpholinoethoxy)-5,6-dihydro-4H-[1,4]oxazepino[5,6,7-de]quinazolin-9-yl)-4-methyl-5-(trifluoromethyl)pyridin-2-amine

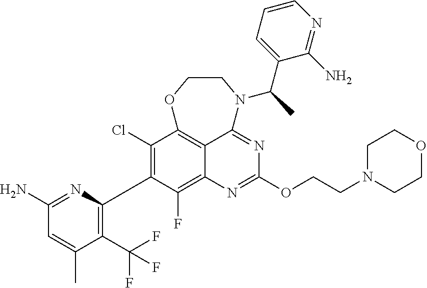

### Synthetic Route

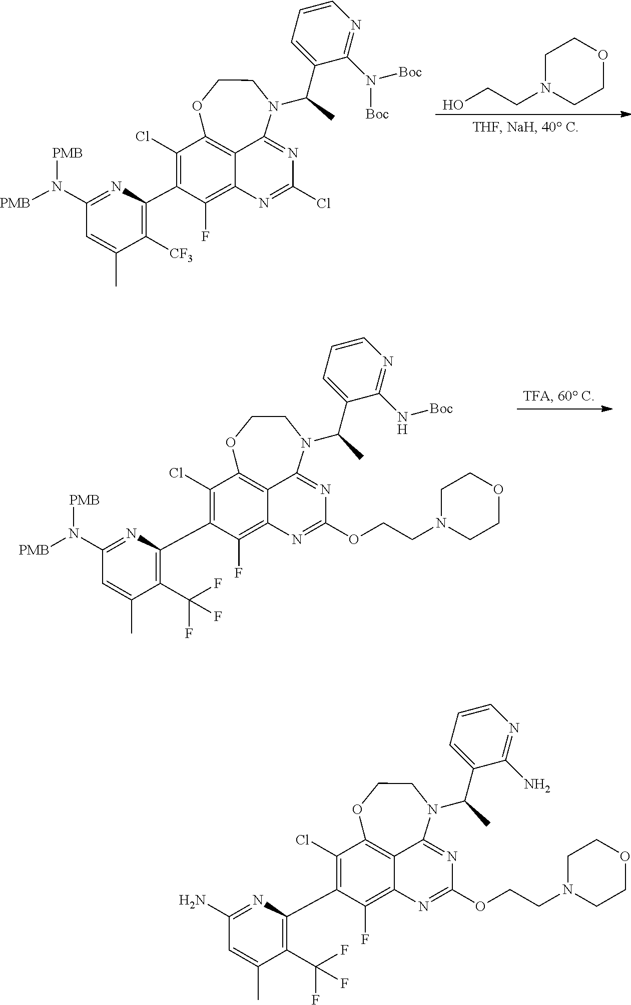

Step 1: tert-butyl (3-((R)-1-((R)-9-(6-(bis(4-methoxybenzyl)amino)-4-methyl-3-(trifluoromethyl)pyridin-2-yl)-8-chloro-10-fluoro-2-(2-morpholinoethoxy)-5,6-dihydro-4H-[1,4]oxazepino[5,6,7-de]quinazolin-4-yl)ethyl)pyridin-2-yl)carbamate

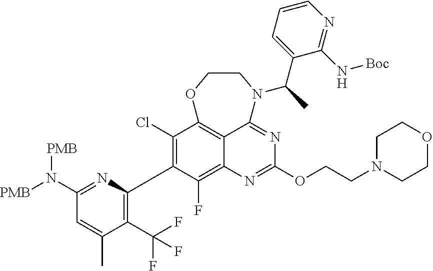

2-morpholinoethan-1-ol (0.15 mmol) was reacted with Di-tert-butyl (3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (50.4 mg, 0.050 mmol) to give the crude title compound as a tan oil which.

Step 2: 6-((9R)-4-((R)-1-(2-aminopyridin-3-yl)ethyl)-8-chloro-10-fluoro-2-((1-methylpyrrolidin-3-yl)methoxy)-5,6-dihydro-4H-[1,4]oxazepino[5,6,7-de]quinazolin-9-yl)-4-methyl-5-(trifluoromethyl)pyridin-2-amine

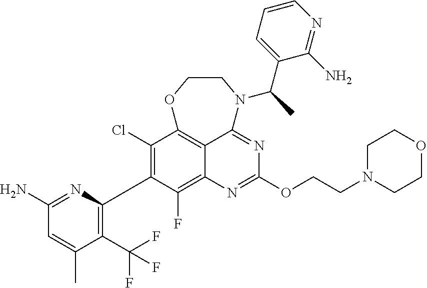

A solution of tert-butyl (3-((R)-1-((R)-9-(6-(bis(4-methoxybenzyl)amino)-4-methyl-3-(trifluoromethyl)pyridin-2-yl)-8-chloro-10-fluoro-2-(2-morpholinoethoxy)-5,6-dihydro-4H-[1,4]oxazepino[5,6,7-de]quinazolin-4-yl)ethyl)pyridin-2-yl)carbamate (˜0.03 mmol) in trifluoroacetic acid (1 mL) was stirred for 5 hours at 55° C. After completion, the reaction mixture was concentrated under vacuum. The product was purified by GILSON 281 and Shimadzu LCMS 2010A Prep-HPLC with the following conditions: Column: Xtimate C18 Column, 25*150 mm, 5 μm; Mobile Phase A: formic acid/water (0.225%), Mobile Phase B: acetonitrile; Flow rate: 25 mL/min; Gradient: 30% B to 70% B in 12.5 min; Wave Length: 254/220 nm to afford 6-((R)-4-((R)-1-(2-aminopyridin-3-yl)ethyl)-8-chloro-10-fluoro-2-(2-morpholinoethoxy)-5,6-dihydro-4H-[1,4]oxazepino[5,6,7-de]quinazolin-9-yl)-4-methyl-5-(trifluoromethyl)pyridin-2-amine (14.0 mg). LC-MS: (ESI, m/z): 663.3 [M+H]⁺, RT1(min) 1.78

Example 138: ¹H NMR (400 MHz, DMSO-d₆, ppm) δ 7.98 (dd, J=5.0, 1.8 Hz, 1H), 7.68 (d, J=7.5 Hz, 1H), 6.79 (s, 2H), 6.74-6.67 (m, 1H), 6.48 (s, 1H), 6.23 (q, J=6.9 Hz, 1H), 5.84 (s, 2H), 4.62 (s, 2H), 4.49-4.38 (m, 1H), 4.32-4.22 (m, 1H), 3.83-3.58 (m, 5H), 3.44-3.34 (m, 2H), 2.94 (d, J=36.4 Hz, 3H), 2.80-2.52 (m, 2H), 2.36 (d, J=2.1 Hz, 3H), 1.57 (d, J=6.9 Hz, 3H). LC-MS: (ESI, m/z): 663.3 [M+H]⁺

Example 139: 6-((9R)-4-((R)-1-(2-aminopyridin-3-yl)ethyl)-8-chloro-10-fluoro-2-(2-(1-methylpyrrolidin-2-yl)ethoxy)-5,6-dihydro-4H-[1,4]oxazepino[5,6,7-de]quinazolin-9-yl)-4-methyl-5-(trifluoromethyl)pyridin-2-amine

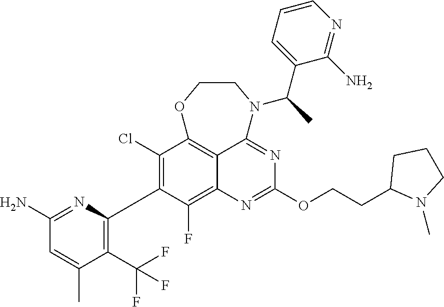

### Synthetic Route

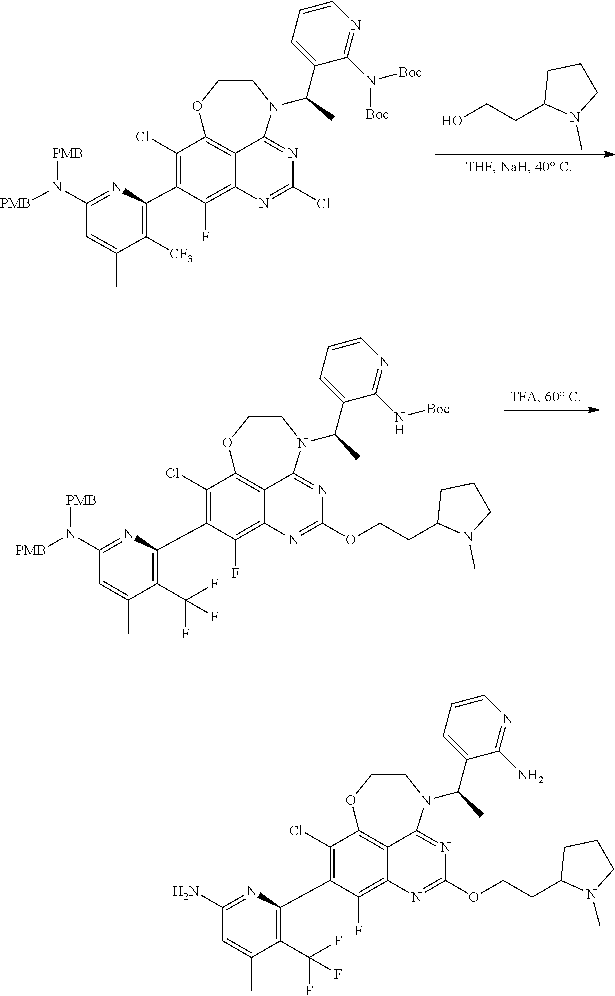

Step 1: tert-butyl (3-((1R)-1-((9R)-9-(6-(bis(4-methoxybenzyl)amino)-4-methyl-3-(trifluoromethyl)pyridin-2-yl)-8-chloro-10-fluoro-2-(2-(1-methylpyrrolidin-2-yl)ethoxy)-5,6-dihydro-4H-[1,4]oxazepino[5,6,7-de]quinazolin-4-yl)ethyl)pyridin-2-yl)carbamate

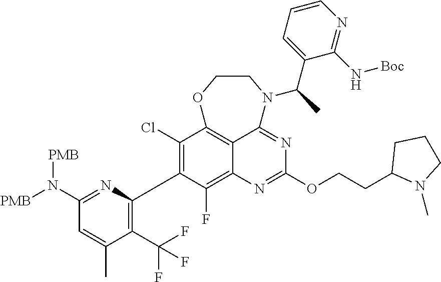

2-(1-methylpyrrolidin-2-yl)ethan-1-ol (0.15 mmol) was reacted with Di-tert-butyl (3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (50.4 mg, 0.050 mmol) to give the crude title compound as a tan oil which was carried forward to the next step.

Step 2: 6-((9R)-4-((R)-1-(2-aminopyridin-3-yl)ethyl)-8-chloro-10-fluoro-2-(2-(1-methylpyrrolidin-2-yl)ethoxy)-5,6-dihydro-4H-[1,4]oxazepino[5,6,7-de]quinazolin-9-yl)-4-, methyl-5-(trifluoromethyl)pyridin-2-amine

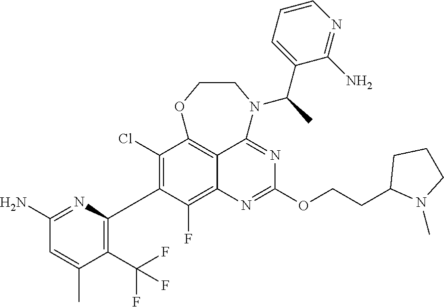

A solution of tert-butyl (3-((1R)-1-((9R)-9-(6-(bis(4-methoxybenzyl)amino)-4-methyl-3-(trifluoromethyl)pyridin-2-yl)-8-chloro-10-fluoro-2-(2-(1-methylpyrrolidin-2-yl)ethoxy)-5,6-dihydro-4H-[1,4]oxazepino[5,6,7-de]quinazolin-4-yl)ethyl)pyridin-2-yl)carbamate (˜0.03 mmol) in trifluoroacetic acid (1 mL) was stirred for 5 hours at 25° C. After completion, the reaction mixture was concentrated under vacuum. The product was purified by GILSON 281 and Shimadzu LCMS 2010A Prep-HPLC with the following conditions: Column: Xtimate C18 Column, 25*150 mm, 5 μm; Mobile Phase A: formic acid/water (0.225%), Mobile Phase B: acetonitrile; Flow rate: 25 mL/min: Gradient: 30% B to 70% B in 12.5 min; Wave Length: 254/220 nm to afford 6-((9R)-4-((R)-1-(2-aminopyridin-3-yl)ethyl)-8-chloro-10-fluoro-2-(2-(1-methylpyrrolidin-2-yl)ethoxy)-5,6-dihydro-4H-[1,4]oxazepino[5,6,7-de]quinazolin-9-yl)-4-methyl-5-(trifluoromethyl)pyridin-2-amine (14.5 mg). LC-MS: (ESI, m/z): 661.3 [M+H]⁺, RT1(min) 2.138

Example 139: ¹H NMR (400 MHz, DMSO-d₆, ppm) δ 7.97 (dd, J=4.9, 1.8 Hz, 1H), 7.64 (d, J=6.7 Hz, 1H), 6.79 (s, 2H), 6.67 (dd, J=7.5, 5.0 Hz, 1H), 6.48 (s, 1H), 6.24 (q, J=6.7 Hz, 1H), 5.70 (s, 2H), 4.53-4.37 (m, 3H), 4.30-4.21 (m, 1H), 3.71-3.60 (m, 1H), 3.42-3.34 (m, 2H), 2.88-2.65 (m, 3H), 2.41-2.14 (m, 6H), 2.02-1.81 (m, 3H), 1.80-1.65 (m, 1H), 1.56 (d, J=6.9 Hz, 3H). LC-MS: (ESI, m/z): 661.3 [M+H]⁺

Example 140: 6-((9R)-4-((R)-1-(2-aminopyridin-3-yl)ethyl)-8-chloro-10-fluoro-2-((4-methylmorpholin-3-yl)methoxy)-5,6-dihydro-4H-[1,4]oxazepino[5,6,7-de]quinazolin-9-yl)-4-methyl-5-(trifluoromethyl)pyridin-2-amine

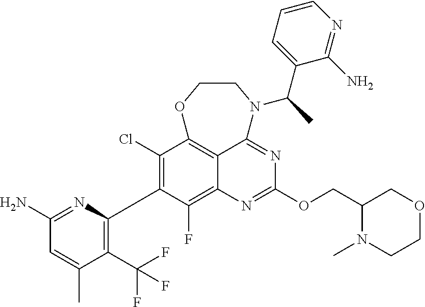

### Synthetic Route

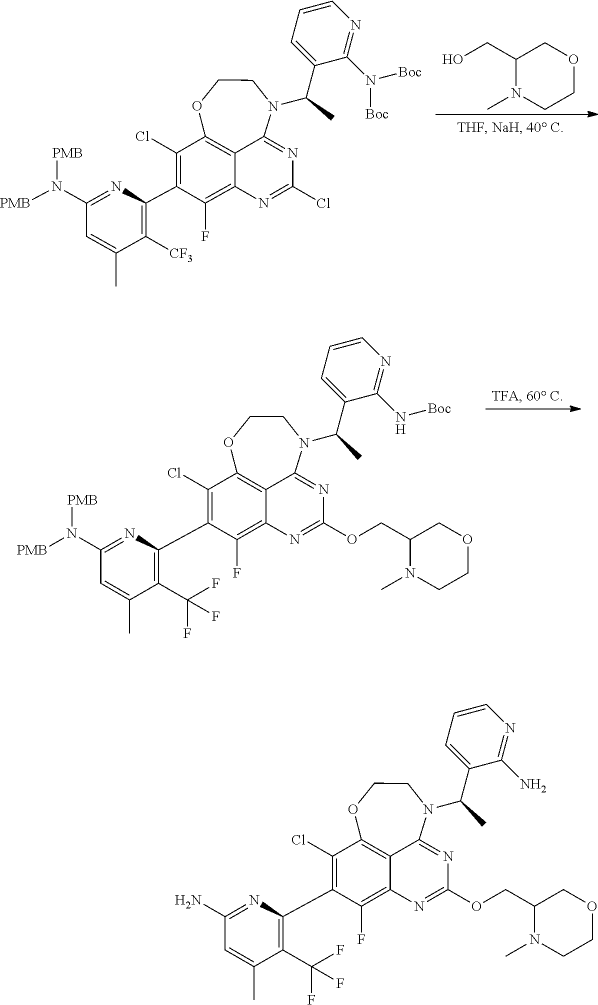

Step 1: tert-butyl (3-((1R)-1-((9R)-9-(6-(bis(4-methoxybenzyl)amino)-4-methyl-3-(trifluoromethyl)pyridin-2-yl)-8-chloro-10-fluoro-2-((4-methylmorpholin-3-yl)methoxy)-5,6-dihydro-4H-[1,4]oxazepino[5,6,7-de]quinazolin-4-yl)ethyl)pyridin-2-yl)carbamate

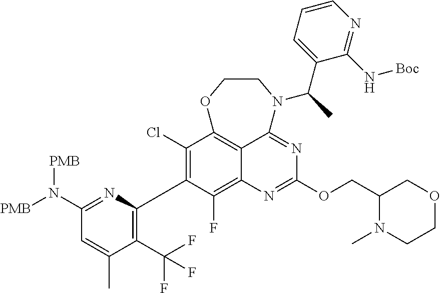

(4-methylmorpholin-3-yl)methanol (0.15 mmol) was reacted with Di-tert-butyl (3-((R)-1-((R)-9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (50.4 mg, 0.050 mmol) to give the crude title compound as a tan oil which was carried forward to the next step.

Step 2: 6-((9R)-4-((R)-1-(2-aminopyridin-3-yl)ethyl)-8-chloro-10-fluoro-2-((4-methylmorpholin-3-yl)methoxy)-5,6-dihydro-4H-[1,4]oxazepino[5,6,7-de]quinazolin-9-yl)-4-methyl-5-(trifluoromethyl)pyridin-2-amine

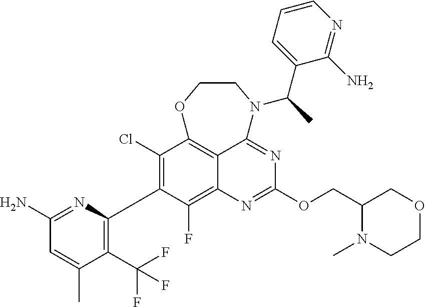

A solution of tert-butyl (3-((1R)-1-((9R)-9-(6-(bis(4-methoxybenzyl)amino)-4-methyl-3-(trifluoromethyl)pyridin-2-yl)-8-chloro-10-fluoro-2-((4-methylmorpholin-3-yl)methoxy)-5,6-dihydro-4H-[1,4]oxazepino[5,6,7-de]quinazolin-4-yl)ethyl)pyridin-2-yl)carbamate (˜0.03 mmol) in trifluoroacetic acid (1 mL) was stirred for 5 hours at 55° C. After completion, the reaction mixture was concentrated under vacuum. The product was purified by GILSON 281 and Shimadzu LCMS 2010A Prep-HPLC with the following conditions: Column: Xtimate C18 Column, 25*150 mm, 5 μm; Mobile Phase A: formic acid/water (0.225%), Mobile Phase B: acetonitrile; Flow rate: 25 mL/min; Gradient: 30% B to 70% B in 12.5 min; Wave Length: 254/220 nm to afford 6-((9R)-4-((R)-1-(2-aminopyridin-3-yl)ethyl)-8-chloro-10-fluoro-2-((4-methylmorpholin-3-yl)methoxy)-5,6-dihydro-4H-[1,4]oxazepino[5,6,7-de]quinazolin-9-yl)-4-methyl-5-(trifluoromethyl)pyridin-2-amine (14.2 mg). LC-MS: (ESI, m/z): 663.3[M+H]⁺, RT1(min) 1.78

Example 140: ¹H NMR (400 MHz, DMSO-d₆, ppm) δ 7.98 (dd, J=4.9, 1.8 Hz, 1H), 7.65 (d, J=9.4 Hz, 1H), 6.80 (s, 2H), 6.68 (dd, J=7.5, 5.0 Hz, 1H), 6.48 (s, 1H), 6.27-6.19 (m, 1H), 5.72 (s, 2H), 4.55 (dd, J=12.3, 4.0 Hz, 1H), 4.42 (dd, J=12.2, 4.8 Hz, 2H), 4.27 (dd, J=11.4, 8.0 Hz, 1H), 3.90 (s, 1H), 3.75 (s, 1H), 3.69-3.32 (m, 7H), 2.81 (s, 3H), 2.36 (d, J=2.1 Hz, 3H), 1.57 (d, J=6.9 Hz, 3H). LC-MS: (ESI, m/z): 663.3[M+H]⁺

Agilent 10-min LCMS method (for examples 141-157): Experiments were performed on an Agilent 1290 UHPLC coupled with Agilent MSD (6140) mass spectrometer using ESI as ionization source. The LC separation was done on a Phenomenex XB-C18, 1.7 um, 50×2.1 mm column at a flow rate of 0.4 ml/minute. MPA (mobile phase A) was water with 0.1% FA and MPB (mobile phase B) was acetonitrile with 0.1% FA. The gradient started at 2% MPB and ended at 98% MPB over 7 min and held at 98% MPB for 1.5 min following an equilibration for 1.5 min. LC column temperature was 40° C. UV absorbance was collected at 220 nm and 254 nm and mass spec full scan was applied to all experiments.

Example 141: 6-((R)-2-((2-oxabicyclo[2.1.1]hexan-4-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

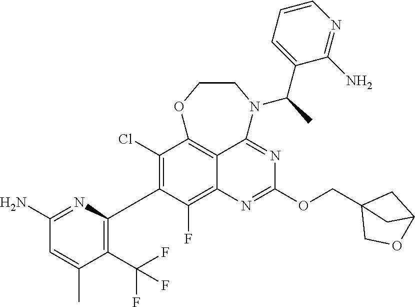

### Synthetic Route

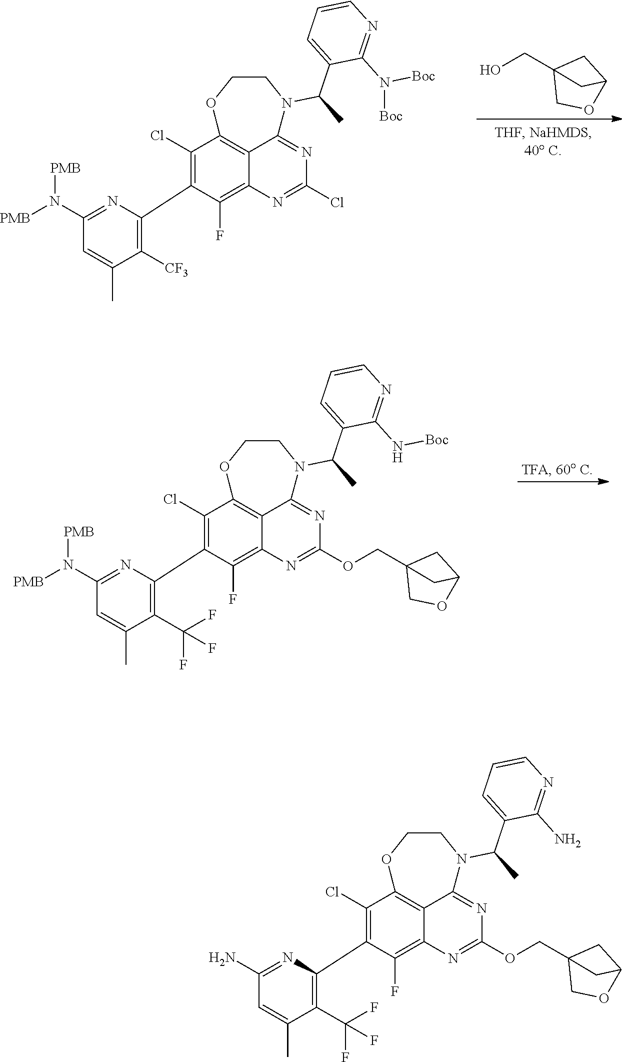

Step 1: tert-butyl (3-((1R)-1-(2-((2-oxabicyclo[2.1.1]hexan-4-yl)methoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate

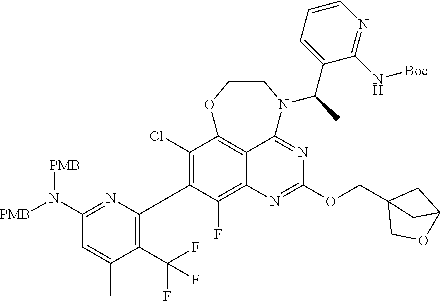

(2-oxabicyclo[2.1.1]hexan-4-yl)methanol (42 mg, 0.37 mmol) was reacted with Di-tert-butyl (3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (152 mg) as a tan oil which was carried forward to the next step. LC-MS: (ESI, m/z): 987.0 [M+H]⁺

Step 2: 6-((R)-2-((2-oxabicyclo[2.1.1]hexan-4-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

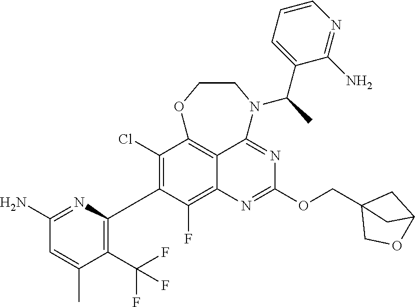

A solution tert-butyl (3-((1R)-1-(2-((2-oxabicyclo[2.1.1]hexan-4-yl)methoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate (100.0 mg, 0.101 mmol) in trifluoroacetic acid (2 mL) was stirred for 3 hours at 50° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Agilent 1290 Infinity II Autoscale Prep LC/MS with the following conditions: Column: XSelect CSH Prep C18, 50*30 mm, 5 μm; Sample Solvent: DMSO: Mobile Phase A: water (0.1% Ammonium hydroxide in Water), Mobile Phase B: acetonitrile; Flow rate: 60 mL/min: Gradient: 20% B to 60% B in 10 min; Wave Length: 240 nm; Column Temp: 25° C.; Agilent 10-min LCMS method retention time (min): 3.61 (undesired atropisomer 4.08) to afford 6-((R)-2-((2-oxabicyclo[2.1.1]hexan-4-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (22.7 mg, 0.035 mmol, 34.7% yield. LC-MS: (ESI, m/z): 646.2 [M+H]⁺.

Example 141: ¹H NMR (500 MHz, DMSO-d6, ppm) δ 7.97 (d, J=6.6 Hz, 1H), 7.62 (d, J=7.6 Hz, 1H), 6.79 (s, 2H), 6.68-6.62 (m, 1H), 6.47 (s, 1H), 6.25 (q, J=6.8 Hz, 1H), 5.69 (s, 2H), 4.67 (s, 2H), 4.50 (s, 1H), 4.43 (dd, J=11.9, 5.8 Hz, 1H), 4.26 (dd, J=11.9, 6.6 Hz, 1H), 3.69-3.61 (m, 1H), 3.60 (s, 2H), 3.42-3.34 (m, 1H), 2.36 (s, 3H), 1.87-1.79 (m, 2H), 1.56 (d, J=6.9 Hz, 3H), 1.52-1.46 (m, 2H). LC-MS: (ESI, m/z): 646.2 [M+H]⁺

Example 142: 4-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)tetrahydro-2H-pyran-4-carbonitrile

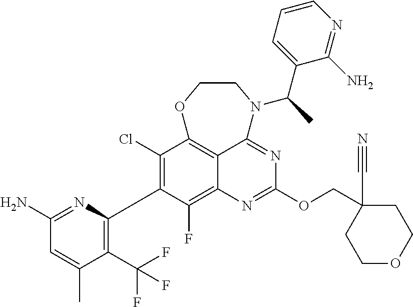

### Synthetic Route

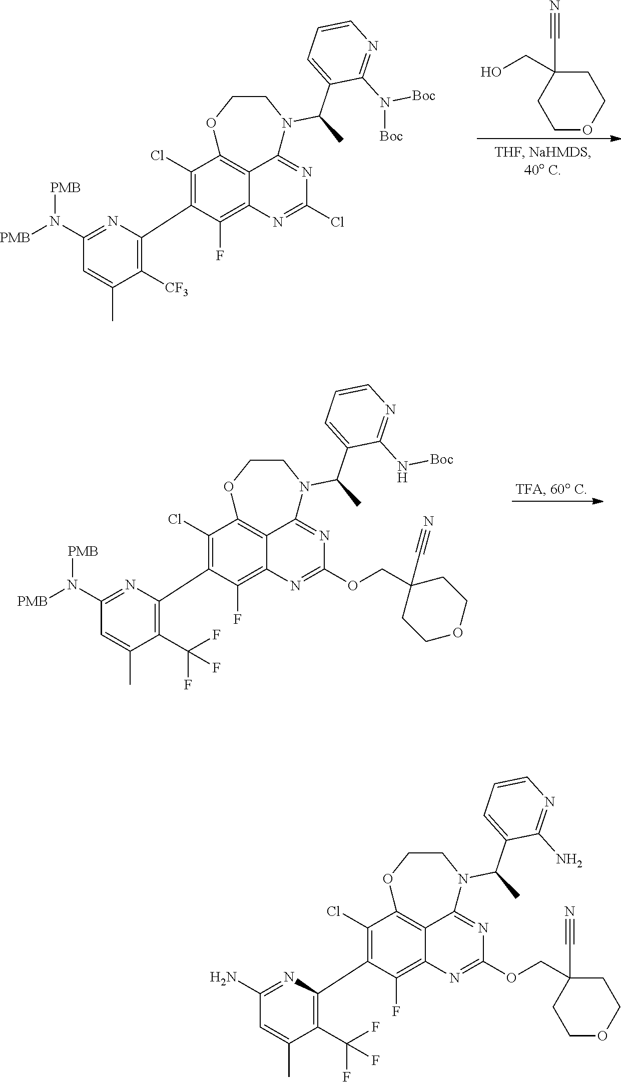

Step 1: tert-butyl (3-((1R)-1-(9-(6-(bis(4-methoxybenzyl)amino)-4-methyl-3-(trifluoromethyl)pyridin-2-yl)-8-chloro-2-((4-cyanotetrahydro-2H-pyran-4-yl)methoxy)-10-fluoro-5,6-dihydro-4H-[1,4]oxazepino[5,6,7-de]quinazolin-4-yl)ethyl)pyridin-2-yl)carbamate

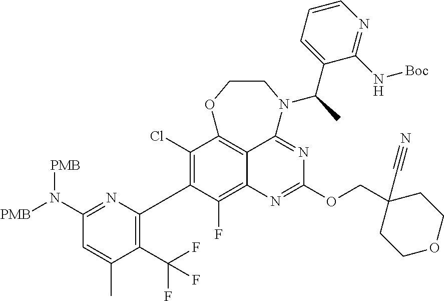

4-(hydroxymethyl)tetrahydro-2H-pyran-4-carbonitrile (52 mg, 0.5 mmol) was reacted with Di-tert-butyl (3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (630.7 mg, 0.57 mmol, 52.3% yield) as a tan oil which. LC-MS: (ESI, m/z): 1014.9 [M+H]⁺

Step 2: 4-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)tetrahydro-2H-pyran-4-carbonitrile

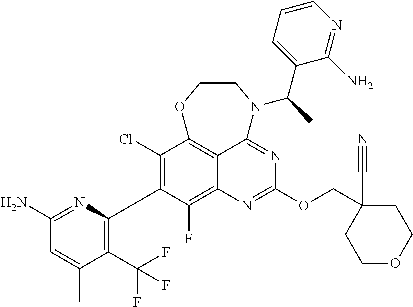

A solution tert-butyl (3-((1R)-1-(9-(6-(bis(4-methoxybenzyl)amino)-4-methyl-3-(trifluoromethyl)pyridin-2-yl)-8-chloro-2-((4-cyanotetrahydro-2H-pyran-4-yl)methoxy)-10-fluoro-5,6-dihydro-4H-[1,4]oxazepino[5,6,7-de]quinazolin-4-yl)ethyl)pyridin-2-yl)carbamate (100.0 mg, 0.099 mmol) in trifluoroacetic acid (2 mL) was stirred for 5 hours at 25° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: XSelect CSH Prep C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 20% B to 60% B in 10 min; Wave Length: 240 nm; Column Temp: 25° C.; Agilent 10-min LCMS method retention time Agilent 10-min LCMS method retention time (min): 3.69 (undesired atropisomer 4.14) to afford 4-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)tetrahydro-2H-pyran-4-carbonitrile (24.47 mg, 0.036 mmol, 36.8% yield). LC-MS: (ESI, m/z): 673.2 [M+H]⁺

Example 142: ¹H NMR (500 MHz, DMSO-da, ppm) δ 7.97 (d, J=4.9 Hz, 1H), 7.63 (d, J=7.6 Hz, 1H), 6.80 (s, 2H), 6.66 (dd, J=7.5, 4.9 Hz, 1H), 6.48 (s, 1H), 6.22 (q, J=7.0 Hz, 1H), 5.68 (s, 2H), 4.52-4.40 (m, 3H), 4.32-4.25 (m, 1H), 3.93 (d, J=12.2 Hz, 2H), 3.71-3.63 (m, 1H), 3.58-3.49 (m, 2H), 3.46-3.38 (m, 1H), 2.36 (s, 3H), 2.03-1.92 (m, 2H), 1.82-1.72 (m, 2H), 1.58 (d, J=6.9 Hz, 3H). LC-MS: (ESI, m/z): 673.2 [M+H]⁺

Example 143: 6-((R)-2-((2-oxabicyclo[2.1.1]hexan-1-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

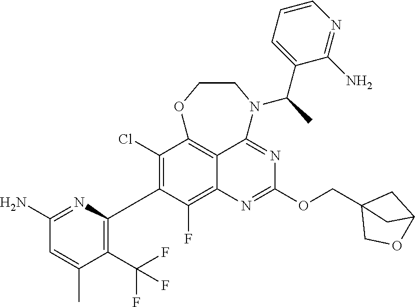

### Synthetic Route

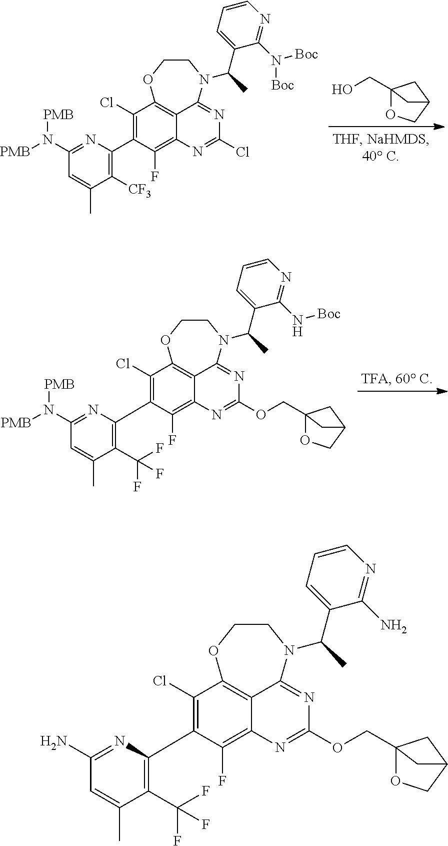

Step 1: tert-butyl (3-((1R)-1-(2-((2-oxabicyclo[2.1.1]hexan-1-yl)methoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate

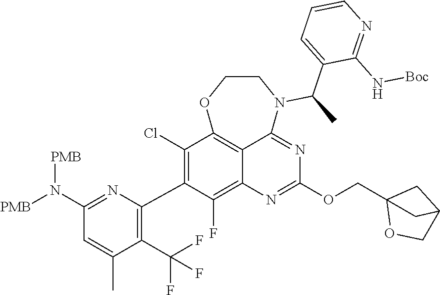

(2-oxabicyclo[2.1.1]hexan-1-yl)methanol (51 mg, 0.44 mmol) was reacted with Di-tert-butyl (3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (150 mg, 0.149 mmol) to give the crude title compound (129 mg) as a tan oil which was carried forward to the next step. LC-MS: (ESI, m/z): 986.9 [M+H]⁺

Step 2: 6-((R)-2-((2-oxabicyclo[2.1.1]hexan-1-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

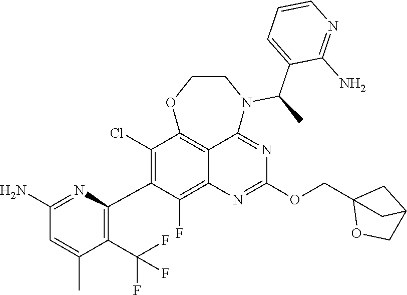

A solution tert-butyl (3-((1R)-1-(2-((2-oxabicyclo[2.1.1]hexan-1-yl)methoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate (100.0 mg, 0.101 mmol) in trifluoroacetic acid (2 mL) was stirred for 5 hours at 25° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: Triart C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 20% B to 60% B in 10 min; Wave Length: 240 nm; Column Temp: 25° C.; Agilent 10-min LCMS method retention time (min): 3.68 (undesired atropisomer 4.17) to afford 6-((R)-2-((2-oxabicyclo[2.1.1]hexan-1-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (21.38 mg, 0.0331 mmol, 32.65% yield. LC-MS: (ESI, m/z): 646.1 [M+H]⁺

Example 143: ¹H NMR (400 MHz, DMSO-d₆, ppm) δ 7.97 (dd, J=5.0, 1.8 Hz, 1H), 7.66 (d, J=7.5 Hz, 1H), 6.80 (s, 2H), 6.69 (dd, J=7.5, 5.0 Hz, 1H), 6.47 (s, 1H), 6.26 (q, J=7.0 Hz, 1H), 5.79 (s, 2H), 4.59 (q, J=12.0 Hz, 2H), 4.46-4.38 (m, 1H), 4.30-4.22 (m, 1H), 3.70 (s, 2H), 3.68-3.59 (m, 1H), 3.41-3.34 (m, 1H), 2.94-2.89 (m, 1H), 2.36 (s, 3H), 1.84 (p, J=3.3 Hz, 2H), 1.57 (d, J=6.9 Hz, 3H), 1.50-1.40 (m, 2H). LC-MS: (ESI, m/z): 646.1 [M+H]⁺

Example 144: 6-((R)-4-((R)-1-(2-aminopyridin-3-yl)ethyl)-8-chloro-10-fluoro-2-((4-methyltetrahydro-2H-pyran-4-yl)methoxy)-5,6-dihydro-4H-[1,4]oxazepino[5,6,7-de]quinazolin-9-yl)-4-methyl-5-(trifluoromethyl)pyridin-2-amine

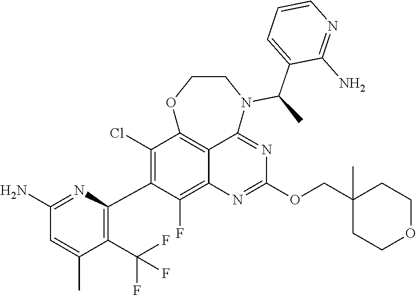

### Synthetic Route

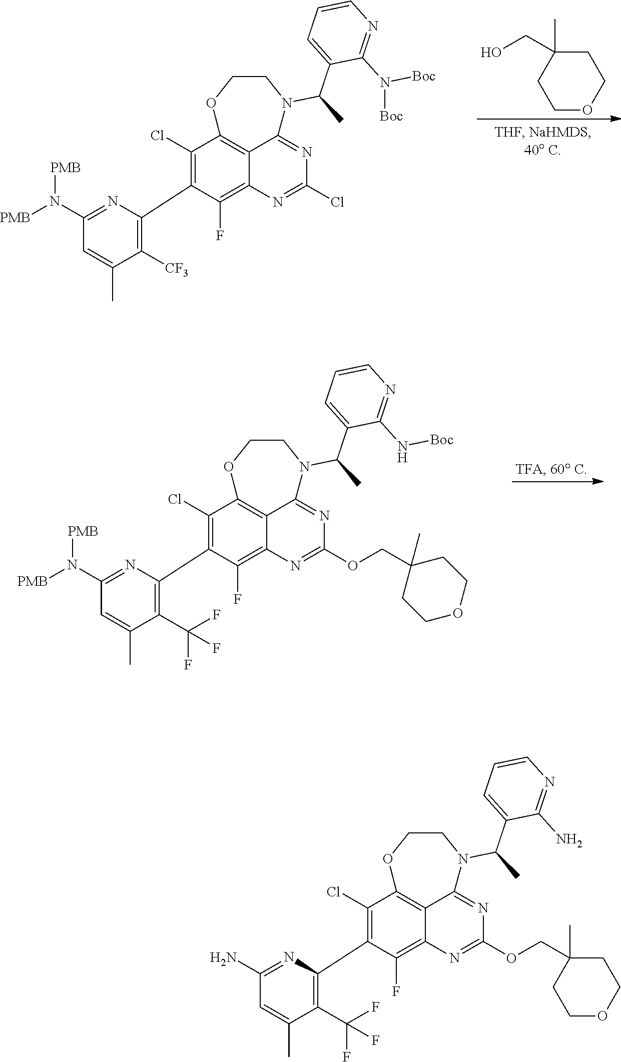

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-((4-methyltetrahydro-2H-pyran-4-yl)methoxy)-5,6-dihydro-4H-[1,4]oxazepino[5,6,7-de]quinazolin-4-yl)ethyl)pyridin-2-yl)carbamate

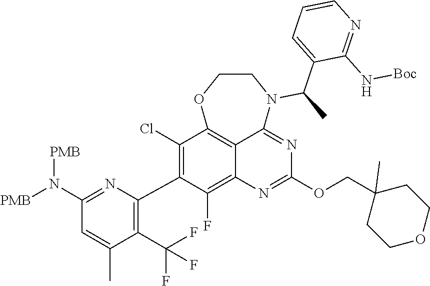

(4-methyltetrahydro-2H-pyran-4-yl)methanol (49 mg, 0.37 mmol) was reacted with Di-tert-butyl (3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg g, 0.124 mmol) to give the crude title compound (113 mg) as a tan oil which was used directly in the next step. LC-MS: (ESI, m/z): 1002.0 [M+H]⁺

Step 2: 6-((R)-4-((R)-1-(2-aminopyridin-3-yl)ethyl)-8-chloro-10-fluoro-2-((4-methyltetrahydro-2H-pyran-4-yl)methoxy)-5,6-dihydro-4H-[1,4]oxazepino[5,6,7-de]quinazolin-9-yl)-4-methyl-5-(trifluoromethyl)pyridin-2-amine

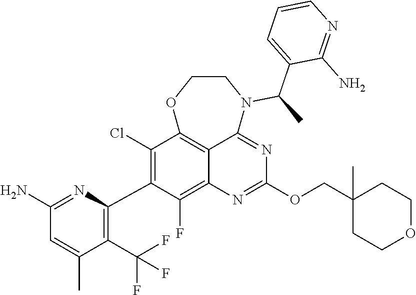

A solution tert-butyl (3-((1R)-1-(9-(6-(bis(4-methoxybenzyl)amino)-4-methyl-3-(trifluoromethyl)pyridin-2-yl)-8-chloro-10-fluoro-2-((4-methyltetrahydro-2H-pyran-4-yl)methoxy)-5,6-dihydro-4H-[1,4]oxazepino[5,6,7-de]quinazolin-4-yl)ethyl)pyridin-2-yl)carbamate (100.0 mg, 0.125 mmol) in trifluoroacetic acid (2 mL) was stirred for 5 hours at 25° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Agilent 1290 Infinity II Autoscale Prep LC/MS with the following conditions: Column: Triart C18, 50*30 mm, 5 μm; Sample Solvent: DMSO: Mobile Phase A: water (0.1% Ammonium hydroxide in Water), Mobile Phase B: acetonitrile; Flow rate: 60 mL/min; Gradient: 30% B to 70% B in 10 min; Wave Length: 254 nm; Column Temp: 25° C.; Agilent 10-min LCMS method retention time (min): 3.80 (undesired atropisomer 4.32) to afford 6-((R)-4-((R)-1-(2-aminopyridin-3-yl)ethyl)-8-chloro-10-fluoro-2-((4-methyltetrahydro-2H-pyran-4-yl)methoxy)-5,6-dihydro-4H-[1,4]oxazepino[5,6,7-de]quinazolin-9-yl)-4-methyl-5-(trifluoromethyl)pyridin-2-amine (30.65 mg, 0.046 mmol, 37.1% yield. LC-MS: (ESI, m/z): 662.2 [M+H]⁺

Example 144: ¹H NMR (500 MHz, DMSO-d₆, ppm) δ 7.96 (d, J=4.9 Hz, 1H), 7.62 (d, J=7.5 Hz, 1H), 6.79 (s, 2H), 6.68-6.62 (m, 1H), 6.47 (s, 1H), 6.24 (q, J=6.9 Hz, 1H), 5.74 (s, 2H), 4.47-4.40 (m, 1H), 4.31-4.24 (m, 1H), 4.20-4.13 (m, 2H), 3.70-3.62 (m, 3H), 3.60-3.52 (m, 2H), 3.43-3.36 (m, 1H), 2.39-2.34 (m, 3H), 1.67-1.59 (m, 2H), 1.57 (d, J=6.9 Hz, 3H), 1.36-1.30 (m, 2H), 1.10 (s, 3H). LC-MS: (ESI, m/z): 662.2 [M+H]⁺

Example 145: (1R,3r)-3-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclobutan-1-ol

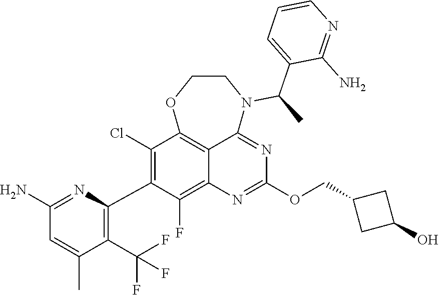

### Synthetic Route

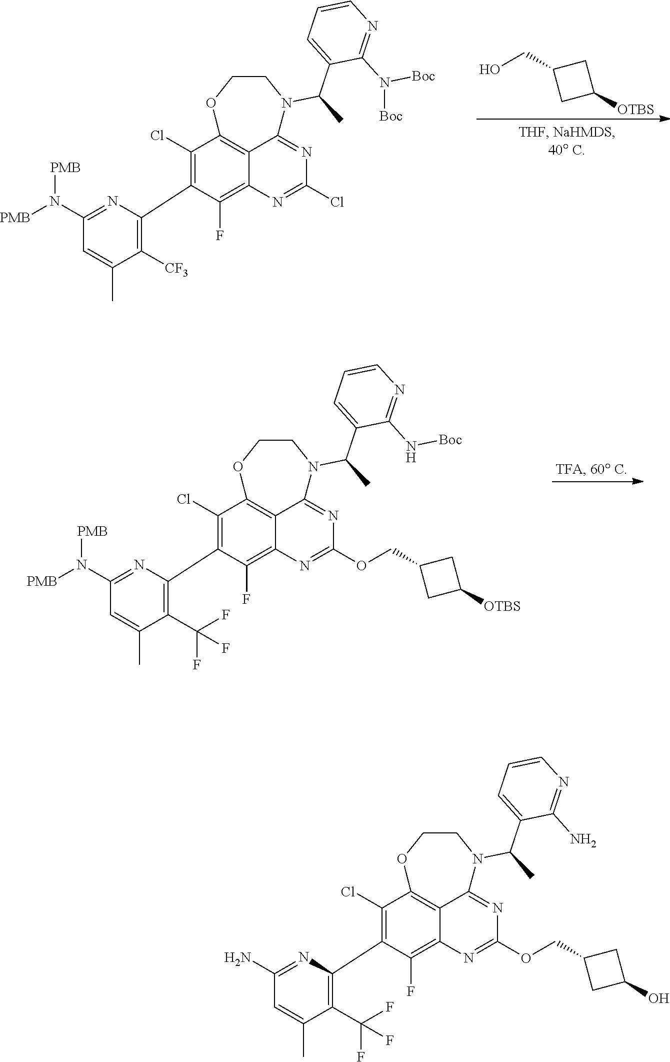

Step 1: tert-butyl (3-((1R)-1-(9-(6-(bis(4-methoxybenzyl)amino)-4-methyl-3-(trifluoromethyl)pyridin-2-yl)-2-(((1r,3R)-3-((tert-butyldimethylsilyl)oxy)cyclobutyl)methoxy)-8-chloro-10-fluoro-5,6-dihydro-4H-[1,4]oxazepino[5,6,7-de]quinazolin-4-yl)ethyl)pyridin-2-yl)carbamate

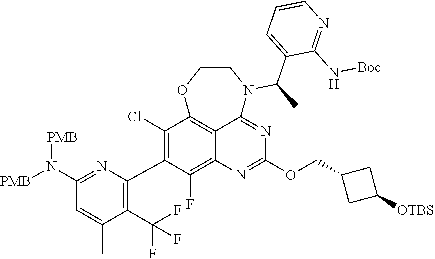

((1r,3r)-3-((tert-butyldimethylsilyl)oxy)cyclobutyl)methanol (81 mg, 0.37 mmol) was reacted with Di-tert-butyl (3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (119 mg) as a tan oil which was used directly in the next step. LC-MS: (ESI, m/z): 1087.95 [M+H]⁺

Step 2: (1R,3r)-3-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclobutan-1-ol

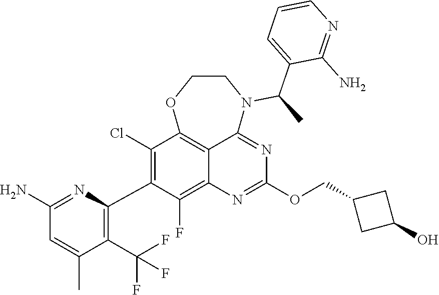

A solution tert-butyl (3-((1R)-1-(9-(6-(bis(4-methoxybenzyl)amino)-4-methyl-3-(trifluoromethyl)pyridin-2-yl)-2-(((1r,3R)-3-((tert-butyldimethylsilyl)oxy)cyclobutyl)methoxy)-8-chloro-10-fluoro-5,6-dihydro-4H-[1,4]oxazepino[5,6,7-de]quinazolin-4-yl)ethyl)pyridin-2-yl)carbamate (100.0 mg, 0.092 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: Triart C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 5% B to 50% B in 10 min; Wave Length: 260 nm; Column Temp: 25° C.; Agilent 10-min LCMS method retention time (min): 3.42 (undesired atropisomer 3.88) to afford (1R,3r)-3-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclobutan-1-ol (14.33 mg, 0.023 mmol, 24.6% yield. LC-MS: (ESI, m/z): 634.2[M+H]⁺

Example 145: ¹H NMR (400 MHz, DMSO-A, ppm) δ 7.97 (dd, J=5.1, 1.9 Hz, 1H), 7.70 (d, J=7.7 Hz, 1H), 6.79 (s, 2H), 6.71 (dd, J=7.5, 5.1 Hz, 1H), 6.47 (s, 1H), 6.26-6.18 (m, 1H), 6.04 (s, 2H), 5.00 (d, J=6.8 Hz, 1H), 4.47-4.40 (m, 1H), 4.36-4.22 (m, 4H), 3.67 (dd, J=15.4, 6.3 Hz, 1H), 3.40 (dd, J=14.2, 6.7 Hz, 1H), 2.39-2.33 (m, 3H), 2.18-1.89 (m, 5H), 1.57 (d, J=6.8 Hz, 3H). LC-MS: (ESI, m/z): 634.2[M+H]⁺

Example 146: (1S,3s)-3-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclobutan-1-ol

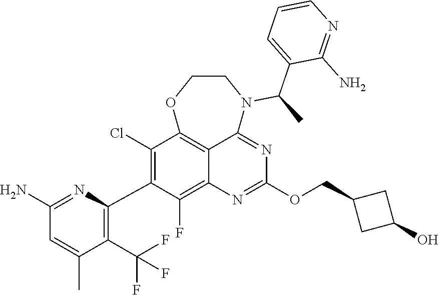

### Synthetic Route

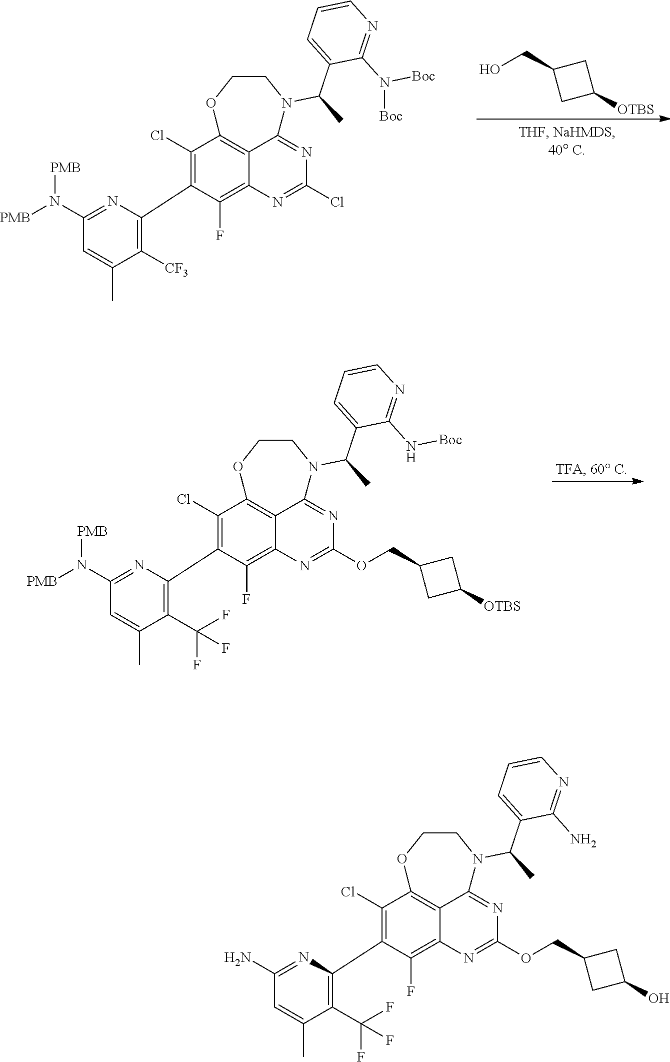

Step 1: tert-butyl (3-((1R)-1-(9-(6-(bis(4-methoxybenzyl)amino)-4-methyl-3-(trifluoromethyl)pyridin-2-yl)-2-(((1s,3S)-3-((tert-butyldimethylsilyl)oxy)cyclobutyl)methoxy)-8-chloro-10-fluoro-5,6-dihydro-4H-[1,4]oxazepino[5,6,7-de]quinazolin-4-yl)ethyl)pyridin-2-yl)carbamate

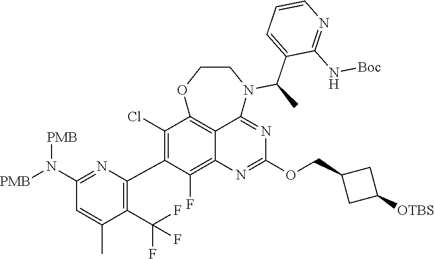

((1s,3s)-3-((tert-butyldimethylsilyl)oxy)cyclobutyl)methanolmethanol (81 mg, 0.374 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (116 mg) as a tan oil which was used directly in the next step. LC-MS: (ESI, m/z): 1087.95 [M+H]⁺

Step 2: (1S,3s)-3-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclobutan-1-ol

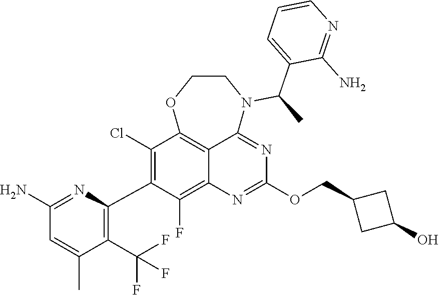

A solution tert-butyl (3-((1R)-1-(9-(6-(bis(4-methoxybenzyl)amino)-4-methyl-3-(trifluoromethyl)pyridin-2-yl)-2-(((1s,3S)-3-((tert-butyldimethylsilyl)oxy)cyclobutyl)methoxy)-8-chloro-10-fluoro-5,6-dihydro-4H-[1,4]oxazepino[5,6,7-de]quinazolin-4-yl)ethyl)pyridin-2-yl)carbamate (100.0 mg, 0.092 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by purified by Interchim PuriFlash 4250 HPLC with the following conditions: Column: XSelect CSH Prep C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 20% B to 60% B in 10 min; Wave Length: 240 nm; Column Temp: 25° C.; Agilent 10-min LCMS method retention time (min): 3.42 (undesired atropisomer 3.90) to afford (1S,3s)-3-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclobutan-1-ol (13.25 mg, 0.021 mmol, 18.2% yield. LC-MS: (ESI, m/z): 634.1 [M+H]⁺

Example 146: ¹H NMR (500 MHz, DMSO-d₆, ppm) δ 7.98 (d, J=5.3 Hz, 1H), 7.74 (d, J=7.6 Hz, 1H), 6.79 (s, 2H), 6.76-6.69 (m, 1H), 6.47 (s, 1H), 6.27-5.99 (m, 3H), 5.02 (s, 1H), 4.47-4.39 (m, 1H), 4.29 (d, J=6.4 Hz, 2H), 4.02-3.93 (m, 1H), 3.73-3.62 (m, 1H), 3.44-3.38 (m, 1H), 2.36 (s, 3H), 2.34-2.27 (m, 2H), 2.19-2.04 (m, 2H), 1.70-1.60 (m, 2H), 1.57 (d, J=6.9 Hz, 3H). LC-MS: (ESI, m/z): 634.1 [M+H]⁺

Example 147: 6-((R)-4-((R)-1-(2-aminopyridin-3-yl)ethyl)-8-chloro-10-fluoro-2-((tetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

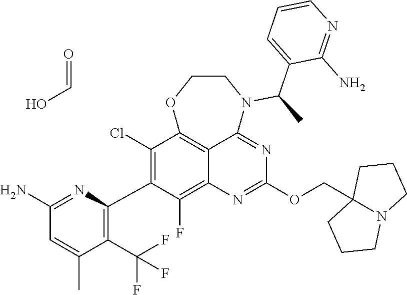

### Synthetic Route

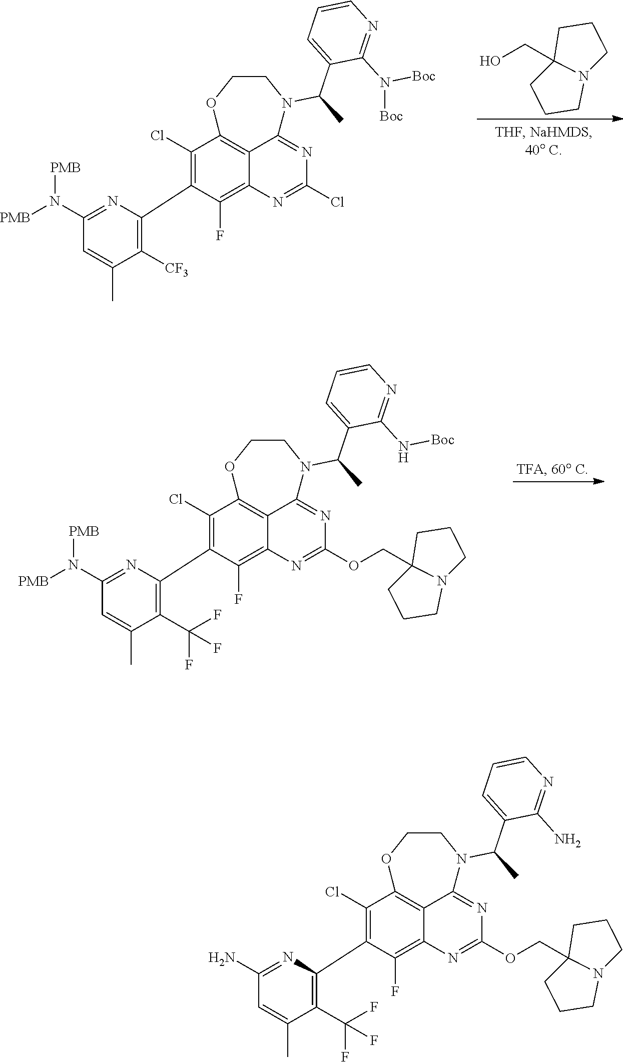

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-((tetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate

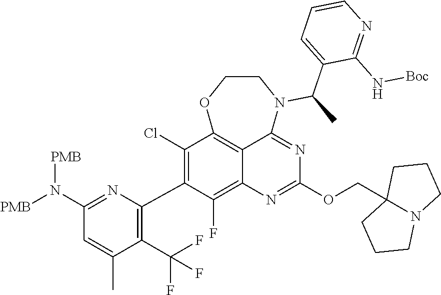

(tetrahydro-1H-pyrrolizin-7a(5H)-yl)methanol (63 mg, 0.45 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (150 mg, 0.149 mmol) to give the crude title compound (121 mg) as a tan oil which was carried forward to the next step. LC-MS: (ESI, m/z): 1012.95 [M+H]⁺

Step 2: 6-((R)-4-((R)-1-(2-aminopyridin-3-yl)ethyl)-8-chloro-10-fluoro-2-((tetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

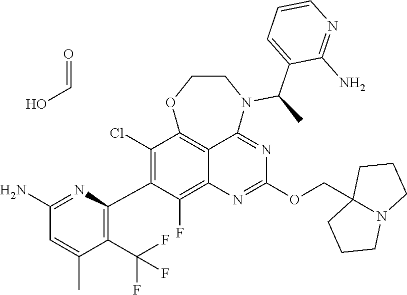

A solution tert-butyl (3-((1R)-1-(9-(6-(bis(4-methoxybenzyl)amino)-4-methyl-3-(trifluoromethyl)pyridin-2-yl)-8-chloro-10-fluoro-2-((tetrahydro-1H-pyrrolizin-7a(5H)-yl)methoxy)-5,6-dihydro-4H-[1,4]oxazepino[5,6,7-de]quinazolin-4-yl)ethyl)pyridin-2-yl)carbamate (100.0 mg, 0.099 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by purified by Interchim PuriFlash 4250 HPLC with the following conditions: Column: XSelect CSH Prep C18, 50*30 mm, 5 μm; Sample Solvent: DMSO; Mobile Phase A: water (0.1% Formic acid in Water), Mobile Phase B: acetonitrile; Flow rate: 60 mL/min; Gradient: 5% B to 50% B in 10 min; Wave Length: 240 nm; Column Temp: 25° C.; Agilent 10-min LCMS method retention time (min): 2.89 (undesired atropisomer 3.30) to afford 6-((R)-4-((R)-1-(2-aminopyridin-3-yl)ethyl)-8-chloro-10-fluoro-2-((tetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (24.84 mg, 0.037 mmol, 37.4% yield a formic acid salt. LC-MS: (ESI, m/z): 673.2[M+H]⁺

Example 147: ¹H NMR (400 MHz, DMSO-d₆, ppm) δ 8.18 (s, 1H), 7.97 (dd, J=5.0, 1.8 Hz, 1H), 7.63 (d, J=7.5 Hz, 1H), 6.79 (s, 2H), 6.67 (dd, J=7.5, 4.9 Hz, 1H), 6.47 (s, 1H), 6.26 (q, J=6.6 Hz, 1H), 5.71 (s, 2H), 4.43 (dd, J=10.8, 6.4 Hz, 1H), 4.25 (dd, J=10.5, 7.2 Hz, 1H), 4.15 (s, 2H), 3.68-3.61 (m, 1H), 3.42-3.32 (m, 2H), 3.09-3.02 (m, 2H), 2.73-2.64 (m, 2H), 2.36 (s, 3H), 2.02-1.92 (m, 2H), 1.91-1.75 (m, 4H), 1.70-1.61 (m, 2H), 1.56 (d, J=6.9 Hz, 3H). LC-MS: (ESI, m/z): 673.2[M+H]⁺

Example 148: 6-((R)-4-((R)-1-(2-aminopyridin-3-yl)ethyl)-8-chloro-10-fluoro-2-((1-(methylsulfonyl)piperidin-4-yl)methoxy)-5,6-dihydro-4H-[1,4]oxazepino[5,6,7-de]quinazolin-9-yl)-4-methyl-5-(trifluoromethyl)pyridin-2-amine

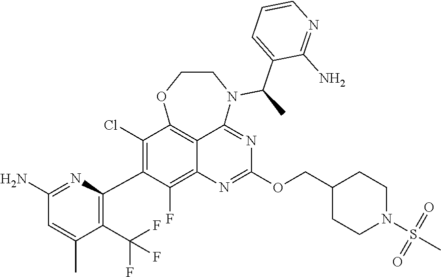

### Synthetic Route

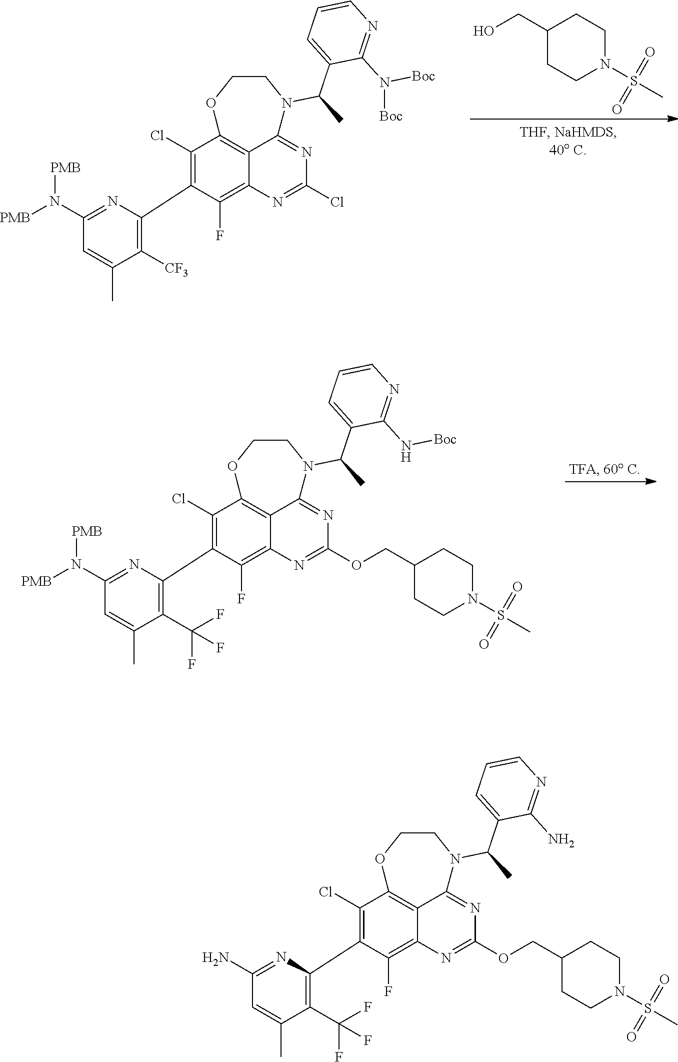

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-((1-(methylsulfonyl)piperidin-4-yl)methoxy)-5,6-dihydro-4H-[1,4]oxazepino[5,6,7-de]quinazolin-4-yl)ethyl)pyridin-2-yl)carbamate

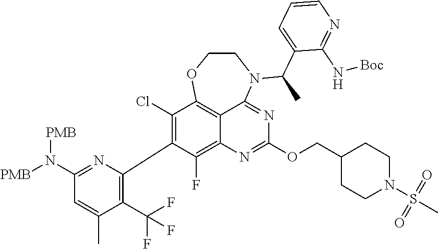

(1-(methylsulfonyl)piperidin-4-yl)methanol (72 mg, 0.37 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg g, 0.124 mmol) to give the crude title compound (119 mg) as a tan oil which was carried forward to the next step. LC-MS: (ESI, m/z): 1064.90 [M+H]⁺

Step 2: 6-((R)-4-((R)-1-(2-aminopyridin-3-yl)ethyl)-8-chloro-10-fluoro-2-((1-(methylsulfonyl)piperidin-4-yl)methoxy)-5,6-dihydro-4H-[1,4]oxazepino[5,6,7-de]quinazolin-9-yl)-4-methyl-5-(trifluoromethyl)pyridin-2-amine

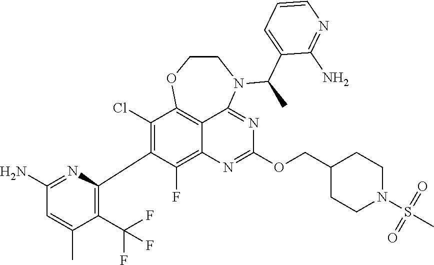

A solution tert-butyl (3-((1R)-1-(9-(6-(bis(4-methoxybenzyl)amino)-4-methyl-3-(trifluoromethyl)pyridin-2-yl)-8-chloro-10-fluoro-2-((1-(methylsulfonyl)piperidin-4-yl)methoxy)-5,6-dihydro-4H-[1,4]oxazepino[5,6,7-de]quinazolin-4-yl)ethyl)pyridin-2-yl)carbamate (100.0 mg, 0.094 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: Triart C18, 50*30 mm, 5 μm; Sample Solvent: DMSO; Mobile Phase A: water (0.1% Formic acid in Water), Mobile Phase B: acetonitrile; Flow rate: 60 mL/min; Gradient: 20% B to 60% B in 10 min; Wave Length: 240 nm; Column Temp: 25° C.; Agilent 10-min LCMS method retention time (min): 3.72 (undesired atropisomer 4.18) to afford 6-((R)-4-((R)-1-(2-aminopyridin-3-yl)ethyl)-8-chloro-10-fluoro-2-((1-(methylsulfonyl)piperidin-4-yl)methoxy)-5,6-dihydro-4H-[1,4]oxazepino[5,6,7-de]quinazolin-9-yl)-4-methyl-5-(trifluoromethyl)pyridin-2-amine (33.98 mg, 0.047 mmol, 49.9% yield. LC-MS: (ESI, m/z): 725.2 [M+H]⁺

Example 148: ¹H NMR (500 MHz, DMSO-d₆, ppm) δ 7.97 (d, J=5.2 Hz, 1H), 7.70 (d, J=8.1 Hz, 1H), 6.80 (s, 2H), 6.74-6.69 (m, 1H), 6.47 (s, 1H), 6.22 (q, J=7.0 Hz, 1H), 6.09 (s, 2H), 4.48-4.40 (m, 1H), 4.34-4.26 (m, 1H), 4.25-4.19 (m, 2H), 3.72-3.65 (m, 1H), 3.63-3.56 (m, 2H), 3.46-3.39 (m, 1H), 2.85 (s, 3H), 2.75-2.69 (m, 2H), 2.36 (s, 3H), 1.95-1.83 (m, 3H), 1.57 (d, J=6.9 Hz, 3H), 1.41-1.29 (m, 2H). LC-MS: (ESI, m/z): 725.2 [M+H]⁺

Example 149: 6-((R)-4-((R)-1-(2-aminopyridin-3-yl)ethyl)-8-chloro-10-fluoro-2-((4-(methoxymethyl)-2-oxabicyclo[2.1.1]hexan-1-yl)methoxy)-5,6-dihydro-4H-[1,4]oxazepino[5,6,7-de]quinazolin-9-yl)-4-methyl-5-(trifluoromethyl)pyridin-2-amine

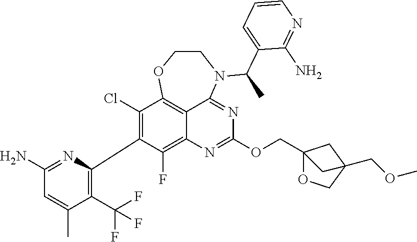

### Synthetic Route

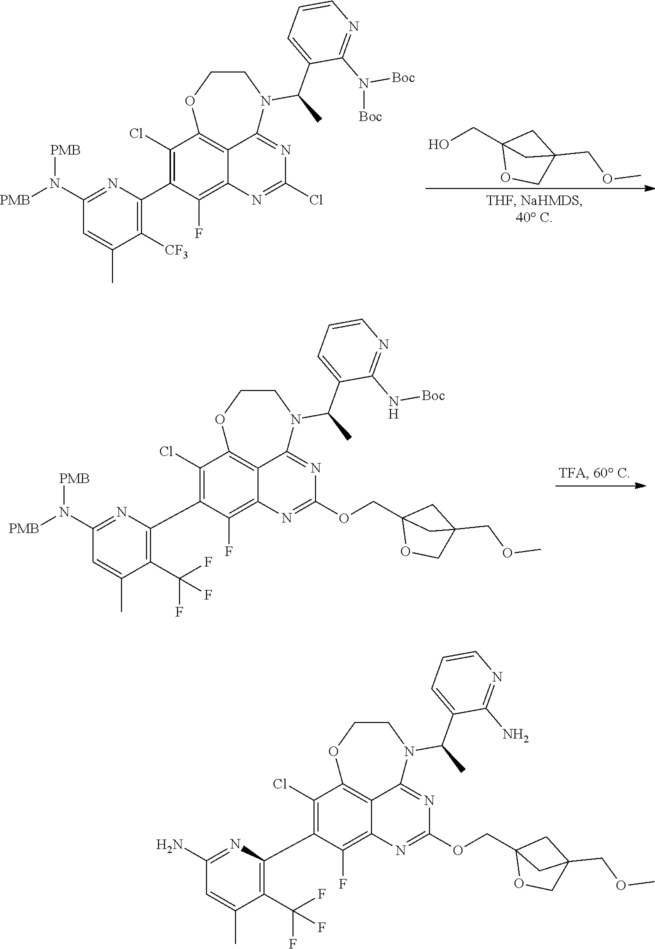

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-((4-(methoxymethyl)-2-oxabicyclo[2.1.1]hexan-1-yl)methoxy)-5,6-dihydro-4H-[1,4]oxazepino[5,6,7-de]quinazolin-4-yl)ethyl)pyridin-2-yl)carbamate

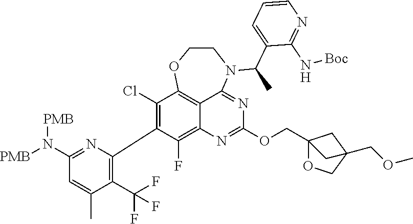

(4-(methoxymethyl)-2-oxabicyclo[2.1.1]hexan-1-yl)methanol (60 mg, 0.38 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (107 mg) as a tan oil which was carried forward to the next step. LC-MS: (ESI, m/z): 1029.85 [M+H]⁺

Step 2: 6-((R)-4-((R)-1-(2-aminopyridin-3-yl)ethyl)-8-chloro-10-fluoro-2-((4-(methoxymethyl)-2-oxabicyclo[2.1.1]hexan-1-yl)methoxy)-5,6-dihydro-4H-[1,4]oxazepino[5,6,7-de]quinazolin-9-yl)-4-methyl-5-(trifluoromethyl)pyridin-2-amine

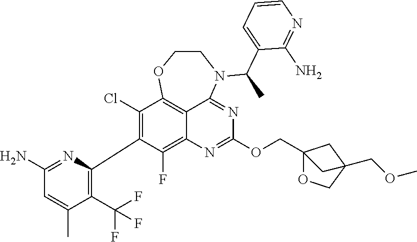

A solution tert-butyl (3-((1R)-1-(9-(6-(bis(4-methoxybenzyl)amino)-4-methyl-3-(trifluoromethyl)pyridin-2-yl)-8-chloro-10-fluoro-2-((4-(methoxymethyl)-2-oxabicyclo[2.1.1]hexan-1-yl)methoxy)-5,6-dihydro-4H-[1,4]oxazepino[5,6,7-de]quinazolin-4-yl)ethyl)pyridin-2-yl)carbamate (100.0 mg, 0.097 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: XSelect CSH Prep C18, 50*30 mm, 5 μm; Sample Solvent: DMSO; Mobile Phase A: water (0.1% Formic acid in Water), Mobile Phase B: acetonitrile; Flow rate: 60 mL/min; Gradient: 5% B to 50% B in 10 min; Wave Length: 210 nm; Column Temp: 25° C.; Agilent 10-min LCMS method retention time (min): 3.72 (undesired atropisomer 4.19) to afford 6-((R)-4-((R)-1-(2-aminopyridin-3-yl)ethyl)-8-chloro-10-fluoro-2-((4-(methoxymethyl)-2-oxabicyclo[2.1.1]hexan-1-yl)methoxy)-5,6-dihydro-4H-[1,4]oxazepino[5,6,7-de]quinazolin-9-yl)-4-methyl-5-(trifluoromethyl)pyridin-2-amine (23.77 mg, 0.034 mmol, 35.5% yield). LC-MS: (ESI, m/z): 690.2 [M+H]⁺

Example 149: ¹H NMR (400 MHz, DMSO-d₆, ppm) δ 7.99 (d, J=5.5 Hz, 1H), 7.82 (d, J=8.2 Hz, 1H), 6.85-6.73 (m, 3H), 6.59-6.39 (m, 2H), 6.23 (q, J=6.8 Hz, 1H), 4.57 (q, J=12.0 Hz, 2H), 4.49-4.41 (m, 1H), 4.36-4.29 (m, 1H), 3.74-3.65 (m, 1H), 3.63-3.58 (m, 4H), 3.44 (dd, J=15.3, 5.8 Hz, 1H), 3.28-3.25 (m, 4H), 2.36 (s, 3H), 1.79-1.74 (m, 2H), 1.61-1.48 (m, 5H). LC-MS: (ESI, m/z): 690.2 [M+H]⁺

Example 150: 6-((R)-4-((R)-1-(2-aminopyridin-3-yl)ethyl)-8-chloro-10-fluoro-2-(2-(1-methoxycyclobutyl)ethoxy)-5,6-dihydro-4H-[1,4]oxazepino[5,6,7-de]quinazolin-9-yl)-4-methyl-5-(trifluoromethyl)pyridin-2-amine

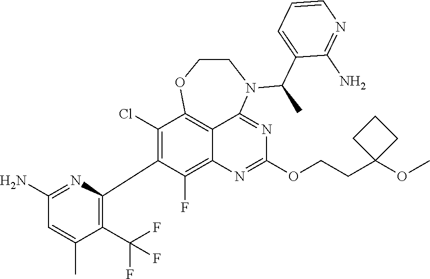

### Synthetic Route

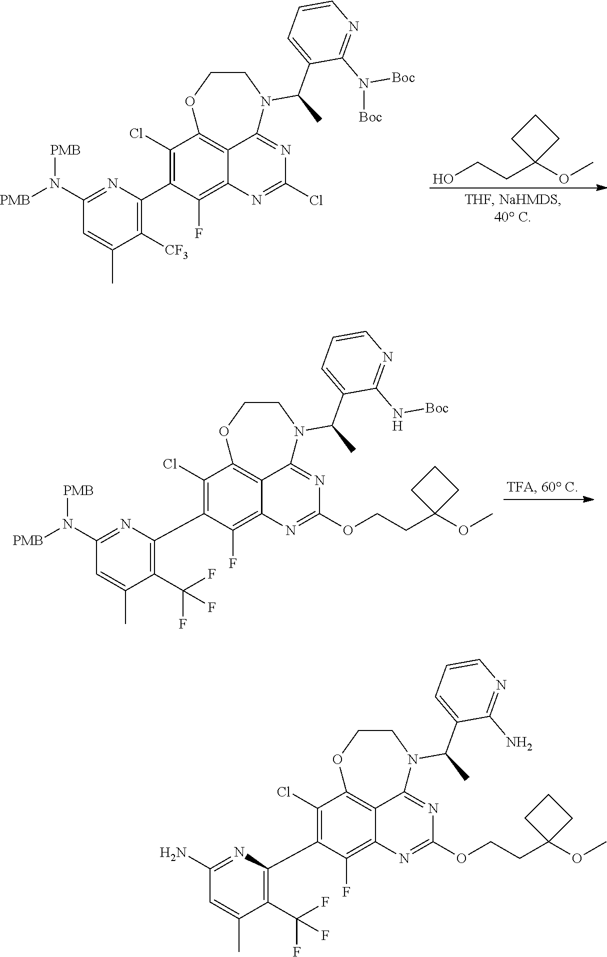

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-(2-(1-methoxycyclobutyl)ethoxy)-5,6-dihydro-4H-[1,4]oxazepino[5,6,7-de]quinazolin-4-yl)ethyl)pyridin-2-yl)carbamate

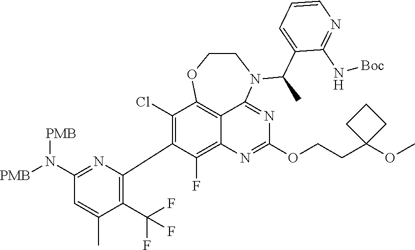

2-(1-methoxycyclobutyl)ethan-1-ol (49 mg, 0.38 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (115 mg) as a tan oil which was carried forward to the next step. LC-MS: (ESI, m/z): 1001.95 [M+H]⁺

Step 2: 6-((R)-4-((R)-1-(2-aminopyridin-3-yl)ethyl)-8-chloro-10-fluoro-2-(2-(1-methoxycyclobutyl)ethoxy)-5,6-dihydro-4H-[1,4]oxazepino[5,6,7-de]quinazolin-9-yl)-4-methyl-5-(trifluoromethyl)pyridin-2-amine

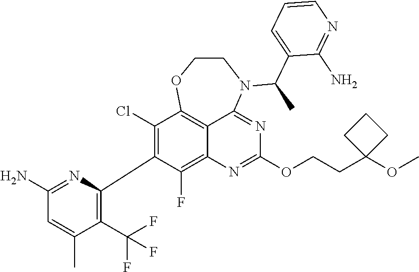

A tert-butyl (3-((1R)-1-(9-(6-(bis(4-methoxybenzyl)amino)-4-methyl-3-(trifluoromethyl)pyridin-2-yl)-8-chloro-10-fluoro-2-(2-(1-methoxycyclobutyl)ethoxy)-5,6-dihydro-4H-[1,4]oxazepino[5,6,7-de]quinazolin-4-yl)ethyl)pyridin-2-yl)carbamate (100.0 mg, 0.10 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Agilent 1290 Infinity II Autoscale Prep LC/MS with the following conditions: Column: XSelect CSH Prep C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 30% B to 70% B in 10 min; Wave Length: 240 nm; Column Temp: 25° C.; Agilent 10-min LCMS method retention time (min): 4.03 (undesired atropisomer 4.56) to afford 6-((R)-4-((R)-1-(2-aminopyridin-3-yl)ethyl)-8-chloro-10-fluoro-2-(2-(1-methoxycyclobutyl)ethoxy)-5,6-dihydro-4H-[1,4]oxazepino[5,6,7-de]quinazolin-9-yl)-4-methyl-5-(trifluoromethyl)pyridin-2-amine (21.0 mg, 0.032 mmol, 31.8% yield). LC-MS: (ESI, m/z): 662.2 [M+H]⁺

Example 150: ¹H NMR (500 MHz, DMSO-d6) δ 7.97 (d, J=4.9 Hz, 1H), 7.63 (d, J=7.5 Hz, 1H), 6.79 (s, 2H), 6.69-6.64 (m, 1H), 6.47 (s, 1H), 6.26 (q, J=6.9 Hz, 1H), 5.67 (s, 2H), 4.46-4.32 (m, 3H), 4.28-4.21 (m, 1H), 3.66-3.58 (m, 1H), 3.39-3.32 (m, 1H), 3.10 (s, 3H), 2.36 (s, 3H), 2.21-2.11 (m, 2H), 2.10-2.00 (m, 2H), 2.00-1.89 (m, 2H), 1.72-1.50 (m, 5H). LC-MS: (ESI, m/z): 662.2 [M+H]⁺

Example 151: 6-((R)-4-((R)-1-(2-aminopyridin-3-yl)ethyl)-8-chloro-10-fluoro-2-(2-fluoroethoxy)-5,6-dihydro-4H-[1,4]oxazepino[5,6,7-de]quinazolin-9-yl)-4-methyl-5-(trifluoromethyl)pyridin-2-amine

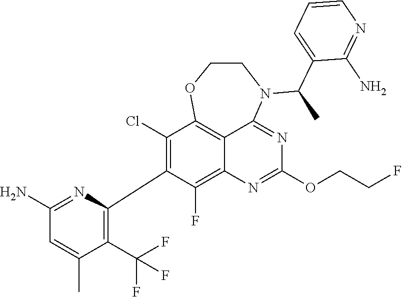

### Synthetic Route

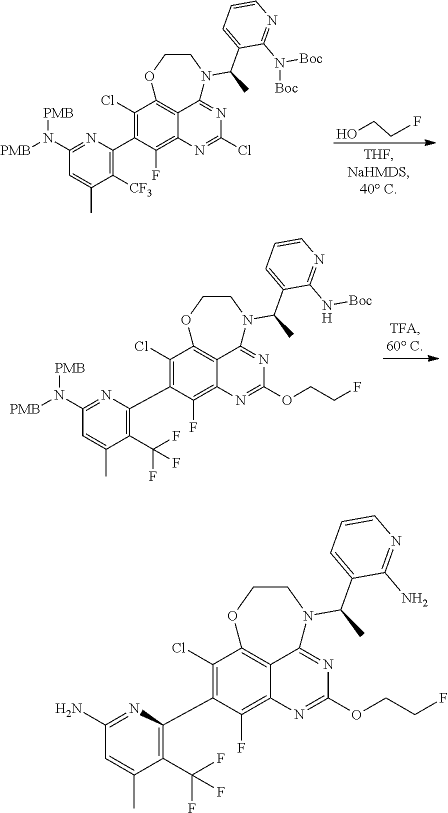

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-(2-fluoroethoxy)-5,6-dihydro-4H-[1,4]oxazepino[5,6,7-de]quinazolin-4-yl)ethyl)pyridin-2-yl)carbamate

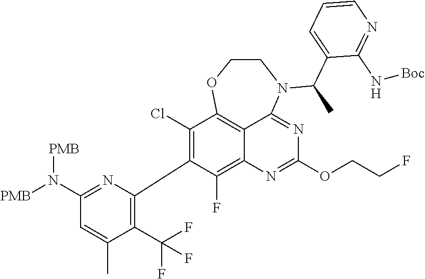

2-fluoroethan-1-ol (24 mg, 0.37 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (105.0 mg) as a tan oil which was carried forward to the next step. LC-MS: (ESI, m/z): 936.00 [M+H]⁺

Step 2: 6-((R)-4-((R)-1-(2-aminopyridin-3-yl)ethyl)-8-chloro-10-fluoro-2-(2-(1-methoxycyclobutyl)ethoxy)-5,6-dihydro-4H-[1,4]oxazepino[5,6,7-de]quinazolin-9-yl)-4-methyl-5-(trifluoromethyl)pyridin-2-amine

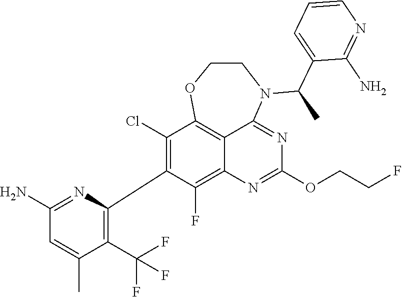

A tert-butyl (3-((1R)-1-(9-(6-(bis(4-methoxybenzyl)amino)-4-methyl-3-(trifluoromethyl)pyridin-2-yl)-8-chloro-10-fluoro-2-(2-fluoroethoxy)-5,6-dihydro-4H-[1,4]oxazepino[5,6,7-de]quinazolin-4-yl)ethyl)pyridin-2-yl)carbamate (100.0 mg, 0.101 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: XSelect CSH Prep C18, 50*30 mm, 5 μm; Sample Solvent: DMSO; Mobile Phase A: water (0.1% Formic acid in Water), Mobile Phase B: acetonitrile; Flow rate: 60 mL/min: Gradient: 5% B to 50% B in 10 min: Wave Length: 240 nm; Column Temp: 25° C.; Agilent 10-min LCMS method retention time (min): 3.57 (undesired atropisomer 4.05) to afford 6-((R)-4-((R)-1-(2-aminopyridin-3-yl)ethyl)-8-chloro-10-fluoro-2-(2-(1-methoxycyclobutyl)ethoxy)-5,6-dihydro-4H-[1,4]oxazepino[5,6,7-de]quinazolin-9-yl)-4-methyl-5-(trifluoromethyl)pyridin-2-amine (27.23 mg, 0.043 mmol, 42.8% yield). LC-MS: (ESI, m/z): 596.1 [M+H]⁺

Example 151: ¹H NMR (400 MHz, DMSO-d₆, ppm) δ 7.97 (dd, J=4.9, 1.8 Hz, 1H), 7.62 (dd, J=7.6, 2.2 Hz, 1H), 6.80 (s, 2H), 6.67 (dd, J=7.5, 4.9 Hz, 1H), 6.47 (s, 1H), 6.24 (q, J=6.4 Hz, 1H), 5.63 (s, 2H), 4.85-4.69 (m, 2H), 4.67-4.54 (m, 2H), 4.45-4.39 (m, 1H), 4.30-4.22 (m, 1H), 3.68-3.59 (m, 1H), 3.41-3.34 (m, 1H), 2.38-2.34 (m, 3H), 1.56 (d, J=6.8 Hz, 3H). LC-MS: (ESI, m/z): 596.1 [M+H]⁺

Example 152: 6-((R)-4-((R)-1-(2-aminopyridin-3-yl)ethyl)-8-chloro-10-fluoro-2-((tetrahydro-2H-pyran-4-yl)methoxy)-5,6-dihydro-4H-[1,4]oxazepino[5,6,7-de]quinazolin-9-yl)-4-methyl-5-(trifluoromethyl)pyridin-2-amine

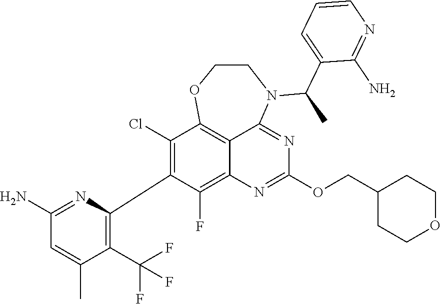

### Synthetic Route

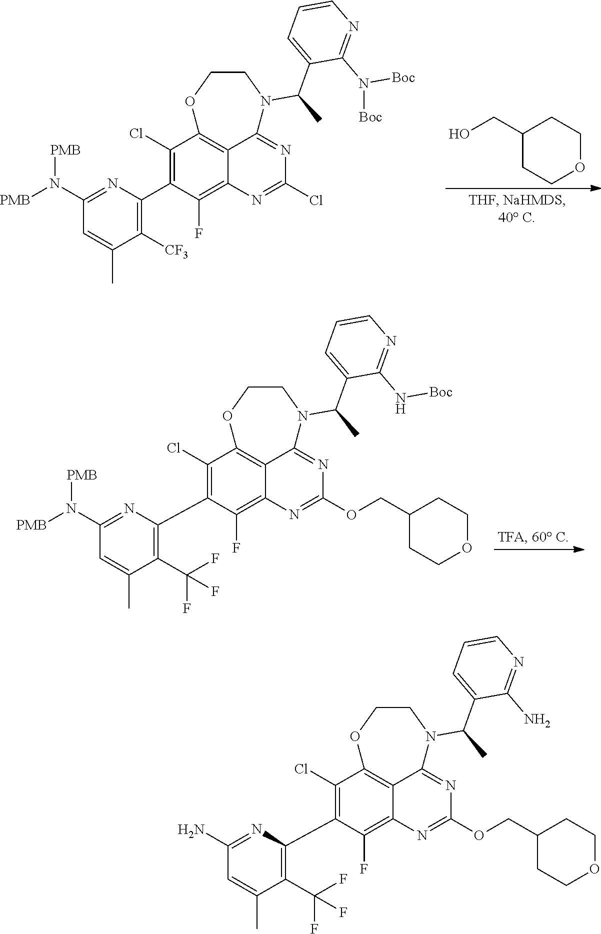

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-((tetrahydro-2H-pyran-4-yl)methoxy)-5,6-dihydro-4H-[1,4]oxazepino[5,6,7-de]quinazolin-4-yl)ethyl)pyridin-2-yl)carbamate

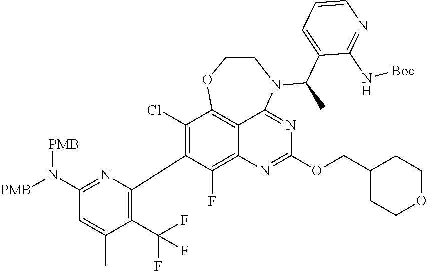

(tetrahydro-2H-pyran-4-yl)methanol (44 mg, 0.38 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (109 mg) as a tan oil which. LC-MS: (ESI, m/z): 987.9 [M+H]⁺

Step 2: 6-((R)-4-((R)-1-(2-aminopyridin-3-yl)ethyl)-8-chloro-10-fluoro-2-((tetrahydro-2H-pyran-4-yl)methoxy)-5,6-dihydro-4H-[1,4]oxazepino[5,6,7-de]quinazolin-9-yl)-4-methyl-5-(trifluoromethyl)pyridin-2-amine

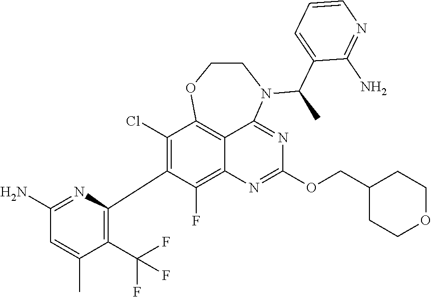

A tert-butyl (3-((1R)-1-(9-(6-(bis(4-methoxybenzyl)amino)-4-methyl-3-(trifluoromethyl)pyridin-2-yl)-8-chloro-10-fluoro-2-((tetrahydro-2H-pyran-4-yl)methoxy)-5,6-dihydro-4H-[1,4]oxazepino[5,6,7-de]quinazolin-4-yl)ethyl)pyridin-2-yl)carbamate (100.0 mg, 0.10 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: Triart C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 30% B to 70% B in 10 min; Wave Length: 240 nm; Column Temp: 25° C.; Agilent 10-min LCMS method retention time (min): 3.66 (undesired atropisomer 4.15) to afford 6-((R)-4-((R)-1-(2-aminopyridin-3-yl)ethyl)-8-chloro-10-fluoro-2-((tetrahydro-2H-pyran-4-yl)methoxy)-5,6-dihydro-4H-[1,4]oxazepino[5,6,7-de]quinazolin-9-yl)-4-methyl-5-(trifluoromethyl)pyridin-2-amine (28.33 mg, 0.044 mmol, 43.2% yield). LC-MS: (ESI, m/z): 648.1 [M+H]⁺

Example 152: ¹H NMR (400 MHz, DMSO-dg, ppm) δ 7.96 (dd, J=4.9, 1.8 Hz, 1H), 7.61 (d, J=7.1 Hz, 1H), 6.79 (s, 2H), 6.66 (dd, J=7.5, 4.9 Hz, 1H), 6.47 (s, 1H), 6.24 (q, J=7.1 Hz, 1H), 5.69 (s, 2H), 4.46-4.39 (m, 1H), 4.30-4.17 (m, 3H), 3.91-3.83 (m, 2H), 3.69-3.60 (m, 1H), 3.42-3.32 (m, 3H), 2.38-2.34 (m, 3H), 2.06-1.97 (m, 1H), 1.71-1.63 (m, 2H), 1.56 (d, J=6.9 Hz, 3H), 1.33 (qd, J=12.3, 4.6 Hz, 2H). LC-MS: (ESI, m/z): 648.1 [M+H]⁺

Example 153: 1-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclopropane-1-carbonitrile

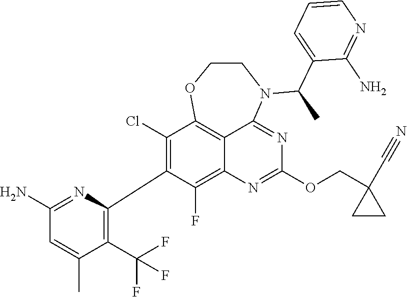

### Synthetic Route

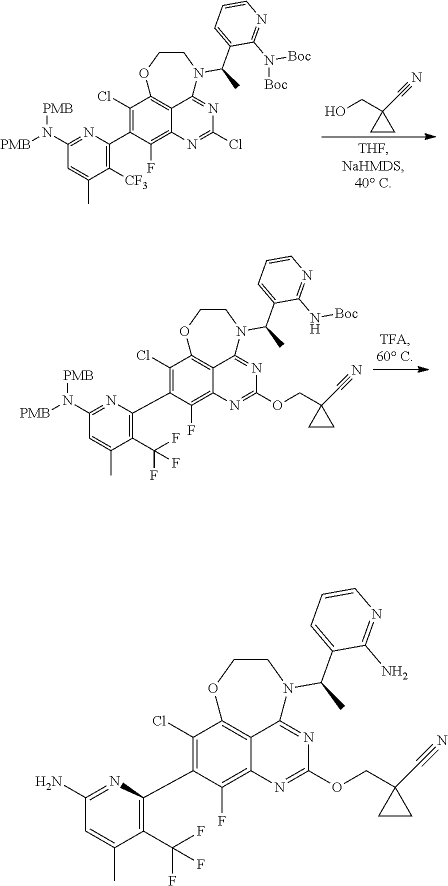

Step 1: tert-butyl (3-((1R)-1-(9-(6-(bis(4-methoxybenzyl)amino)-4-methyl-3-(trifluoromethyl)pyridin-2-yl)-8-chloro-2-((1-cyanocyclopropyl)methoxy)-10-fluoro-5,6-dihydro-4H-[1,4]oxazepino[5,6,7-de]quinazolin-4-yl)ethyl)pyridin-2-yl)carbamate

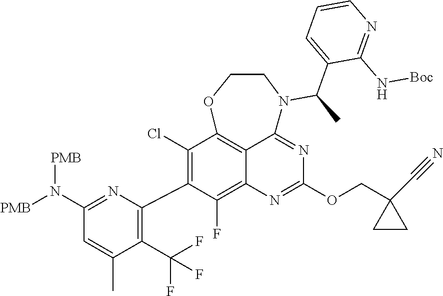

1-(hydroxymethyl)cyclopropane-1-carbonitrile (37 mg, 0.381 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (119 mg) as a tan oil which. LC-MS: (ESI, m/z): 968.9 [M+H]⁺

Step 2: 1-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclopropane-1-carbonitrile

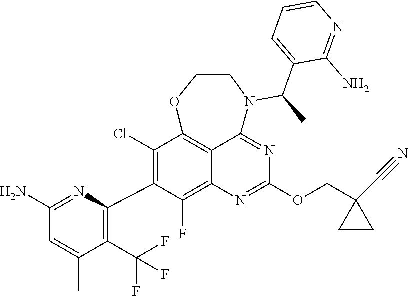

A tert-butyl (3-((1R)-1-(9-(6-(bis(4-methoxybenzyl)amino)-4-methyl-3-(trifluoromethyl)pyridin-2-yl)-8-chloro-2-((1-cyanocyclopropyl)methoxy)-10-fluoro-5,6-dihydro-4H-[1,4]oxazepino[5,6,7-de]quinazolin-4-yl)ethyl)pyridin-2-yl)carbamate (100.0 mg, 0.103 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: XSelect CSH Prep C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 20% B to 60% B in 10 min; Wave Length: 240 nm; Column Temp: 25° C.; Agilent 10-min LCMS method retention time (min): 3.76 (undesired atropisomer 4.21) to afford 1-((((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)methyl)cyclopropane-1-carbonitrile (26.8 mg, 0.043 mmol, 41.3% yield). LC-MS: (ESI, m/z): 629.1 [M+H]⁺

Example 153: ¹H NMR (400 MHz, DMSO-d6, ppm) δ 7.97 (dd, J=4.9, 1.7 Hz, 1H), 7.66-7.59 (m, 1H), 6.81 (s, 2H), 6.67 (dd, J=7.5, 4.9 Hz, 1H), 6.50-6.45 (m, 1H), 6.23 (q, J=6.8 Hz, 1H), 5.61 (s, 2H), 4.47-4.32 (m, 3H), 4.26 (dd, J=11.2, 7.3 Hz, 1H), 3.64 (dd, J=15.5, 6.9 Hz, 1H), 3.44-3.34 (m, 1H), 2.36 (tt, J=2.1, 1.2 Hz, 3H), 1.57 (d, J=6.8 Hz, 3H), 1.41-1.28 (m, 2H), 1.28-1.21 (m, 2H). LC-MS: (ESI, m/z): 629.1 [M+H]⁺

Example 154: 6-((R)-4-((R)-1-(2-aminopyridin-3-yl)ethyl)-8-chloro-10-fluoro-2-((1-(fluoromethyl)-2-oxabicyclo[2.1.1]hexan-4-yl)methoxy)-5,6-dihydro-4H-[1,4]oxazepino[5,6,7-de]quinazolin-9-yl)-4-methyl-5-(trifluoromethyl)pyridin-2-amine

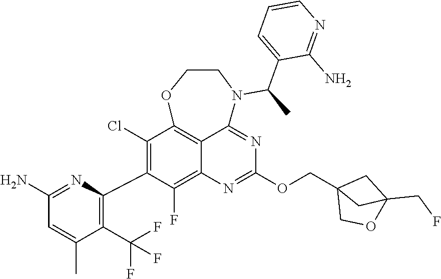

### Synthetic Route

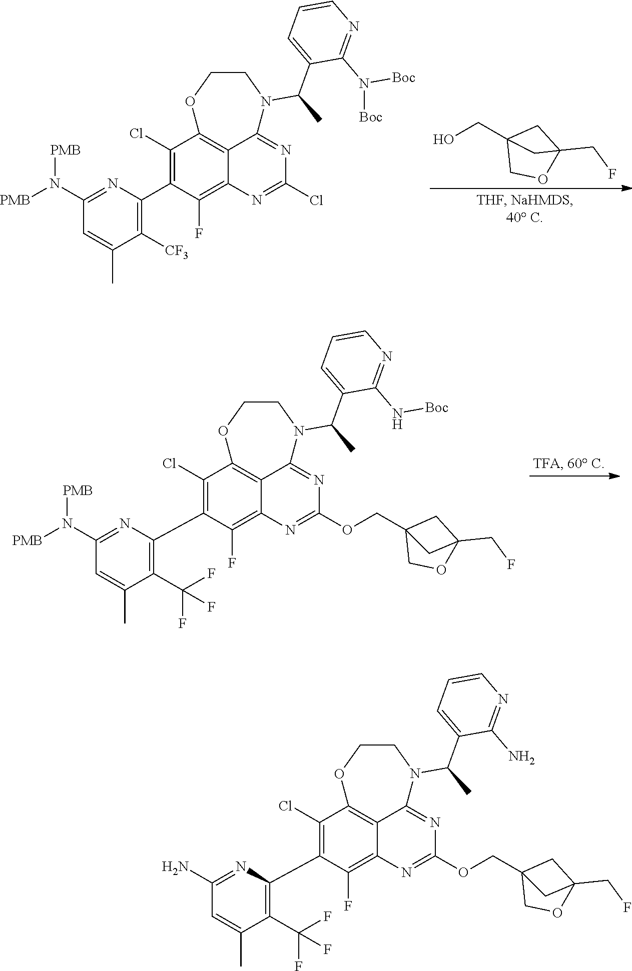

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-((1-(fluoromethyl)-2-oxabicyclo[2.1.1]hexan-4-yl)methoxy)-5,6-dihydro-4H-[1,4]oxazepino[5,6,7-de]quinazolin-4-yl)ethyl)pyridin-2-yl)carbamate

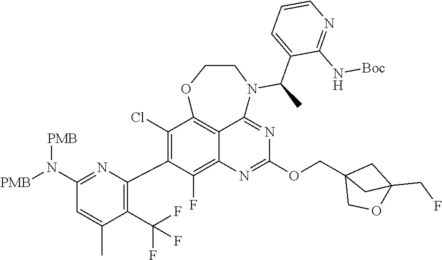

(1-(fluoromethyl)-2-oxabicyclo[2.1.1]hexan-4-yl)methanol (55 mg, 0.38 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (119 mg) as a tan oil which. LC-MS: (ESI, m/z): 1017.95 [M+H]⁺

Step 2: 6-((R)-4-((R)-1-(2-aminopyridin-3-yl)ethyl)-8-chloro-10-fluoro-2-((1-(fluoromethyl)-2-oxabicyclo[2.1.1]hexan-4-yl)methoxy)-5,6-dihydro-4H-[1,4]oxazepino[5,6,7-de]quinazolin-9-yl)-4-methyl-5-(trifluoromethyl)pyridin-2-amine

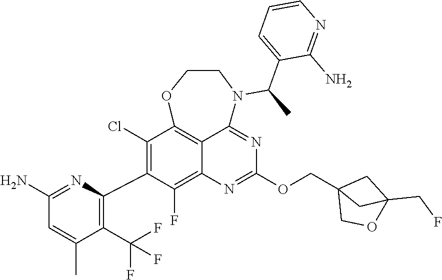

A tert-butyl (3-((1R)-1-(9-(6-(bis(4-methoxybenzyl)amino)-4-methyl-3-(trifluoromethyl)pyridin-2-yl)-8-chloro-10-fluoro-2-((1-(fluoromethyl)-2-oxabicyclo[2.1.1]hexan-4-yl)methoxy)-5,6-dihydro-4H-[1,4]oxazepino[5,6,7-de]quinazolin-4-yl)ethyl)pyridin-2-yl)carbamate (100.0 mg, 0.098 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Agilent 1290 Infinity II Autoscale Prep LC/MS with the following conditions: Column: XSelect CSH Prep C18, 50*30 mm, 5 μm; Sample Solvent: DMSO; Mobile Phase A: water (0.1% Ammonium hydroxide in Water), Mobile Phase B: acetonitrile; Flow rate: 60 mL/min; Gradient: 30% B to 70% B in 10 min; Wave Length: 240 nm; Column Temp: 25° C.; Agilent 10-min LCMS method retention time (min): 3.73 (undesired atropisomer 4.20) to afford 6-((R)-4-((R)-1-(2-aminopyridin-3-yl)ethyl)-8-chloro-10-fluoro-2-((1-(fluoromethyl)-2-oxabicyclo[2.1.1]hexan-4-yl)methoxy)-5,6-dihydro-4H-[1,4]oxazepino[5,6,7-de]quinazolin-9-yl)-4-methyl-5-(trifluoromethyl)pyridin-2-amine (55.9 mg, 0.0824 mmol, 84.0% yield). LC-MS: (ESI, m/z): 678.1 [M+H]⁺

Example 154: ¹H NMR (500 MHz, DMSO-d₆, ppm) δ 7.96 (d, J=4.9 Hz, 1H), 7.62 (d, J=7.5 Hz, 1H), 6.79 (s, 2H), 6.66 (dd, J=7.5, 4.9 Hz, 1H), 6.47 (s, 1H), 6.24 (q, J=6.9 Hz, 1H), 5.70 (s, 2H), 4.67 (s, 3H), 4.57 (s, 1H), 4.46-4.40 (m, 1H), 4.30-4.22 (m, 1H), 3.72 (s, 2H), 3.69-3.60 (m, 1H), 3.43-3.36 (m, 1H), 2.36 (s, 3H), 1.91-1.81 (m, 2H), 1.66-1.50 (m, 5H). LC-MS: (ESI, m/z): 678.1 [M+H]⁺

Example 155: 6-((R)-4-((R)-1-(2-aminopyridin-3-yl)ethyl)-8-chloro-10-fluoro-2-methoxy-5,6-dihydro-4H-[1,4]oxazepino[5,6,7-de]quinazolin-9-yl)-4-methyl-5-(trifluoromethyl)pyridin-2-amine

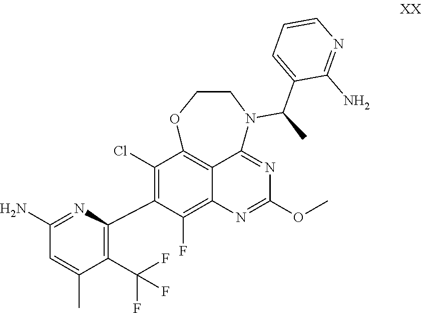

### Synthetic Route

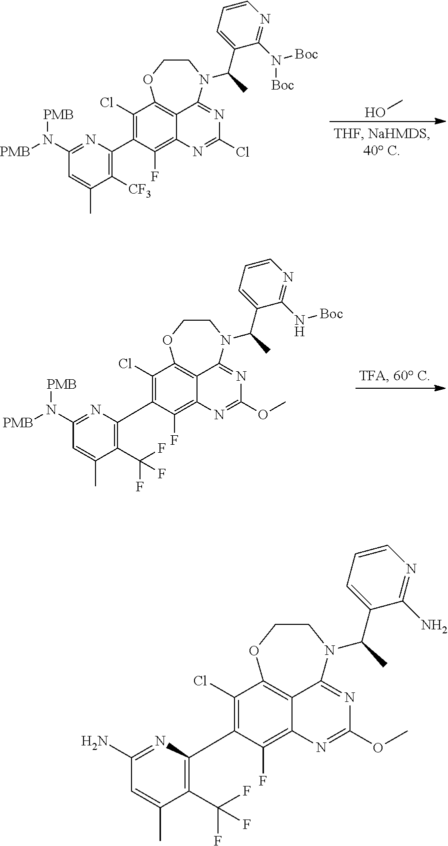

Step 1: tert-butyl (3-((1R)-1-(9-(6-(bis(4-methoxybenzyl)amino)-4-methyl-3-(trifluoromethyl)pyridin-2-yl)-8-chloro-10-fluoro-2-methoxy-5,6-dihydro-4H-[1,4]oxazepino[5,6,7-de]quinazolin-4-yl)ethyl)pyridin-2-yl)carbamate

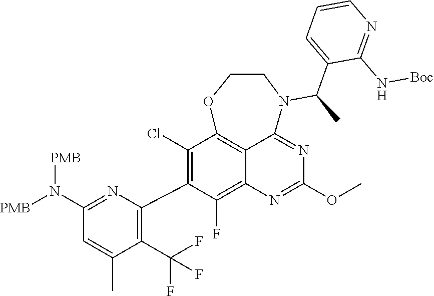

methanol (12 mg, 0.37 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (108 mg) as a tan oil which was carried forward to the next step. LC-MS: (ESI, m/z): 903.95 [M+H]⁺

Step 2:6-((R)-4-((R)-1-(2-aminopyridin-3-yl)ethyl)-8-chloro-10-fluoro-2-methoxy-5,6-dihydro-4H-[1,4]oxazepino[5,6,7-de]quinazolin-9-yl)-4-methyl-5-(trifluoromethyl)pyridin-2-amine

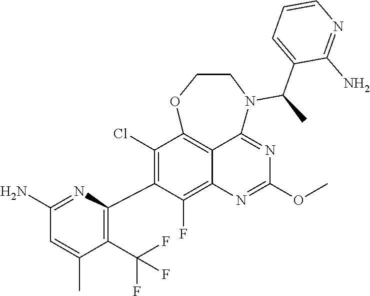

A tert-butyl (3-((1R)-1-(9-(6-(bis(4-methoxybenzyl)amino)-4-methyl-3-(trifluoromethyl)pyridin-2-yl)-8-chloro-10-fluoro-2-methoxy-5,6-dihydro-4H-[1,4]oxazepino[5,6,7-de]quinazolin-4-yl)ethyl)pyridin-2-yl)carbamate (100 mg, 0.11 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Agilent 1290 Infinity II Autoscale Prep LC/MS with the following conditions: Column: XSelect CSH Prep C18, 50*30 mm, 5 μm; Sample Solvent: DMSO: Mobile Phase A: water (0.1% Formic acid in Water), Mobile Phase B: acetonitrile; Flow rate: 60 mL/min; Gradient: 5% B to 50% B in 10 min; Wave Length: 240 nm; Column Temp: 25° C.; Agilent 10-min LCMS method retention time (min): 3.41 (undesired atropisomer 4.91) to afford 6-((R)-4-((R)-1-(2-aminopyridin-3-yl)ethyl)-8-chloro-10-fluoro-2-methoxy-5,6-dihydro-4H-[1,4]oxazepino[5,6,7-de]quinazolin-9-yl)-4-methyl-5-(trifluoromethyl)pyridin-2-amine (15.47 mg, 0.027 mmol, 24.8% yield). LC-MS: (ESI, m/z): 564.1 [M+H]⁺

Example 155: ¹H NMR (400 MHz, DMSO-d₆, ppm) δ 7.96 (dd, J=4.9, 1.8 Hz, 1H), 7.63 (dd, J=7.9, 1.8 Hz, 1H), 6.79 (s, 2H), 6.66 (dd, J=7.4, 4.9 Hz, 1H), 6.47 (s, 1H), 6.27 (q, J=6.9 Hz, 1H), 5.69 (s, 2H), 4.43 (dd, J=12.3, 6.4 Hz, 1H), 4.25 (dd, J=12.1, 7.0 Hz, 1H), 3.94 (s, 3H), 3.68-3.59 (m, 1H), 3.40-3.36 (m, 1H), 2.38-2.34 (m, 3H), 1.55 (d, J=6.9 Hz, 3H). LC-MS: (ESI, m/z): 564.1 [M+H]⁺

Example 156: 2-(((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)ethan-1-ol

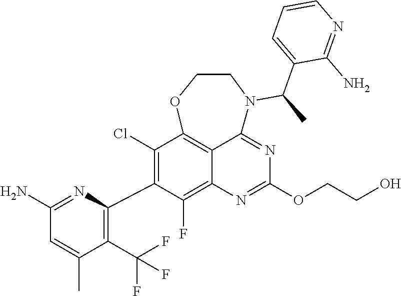

### Synthetic Route

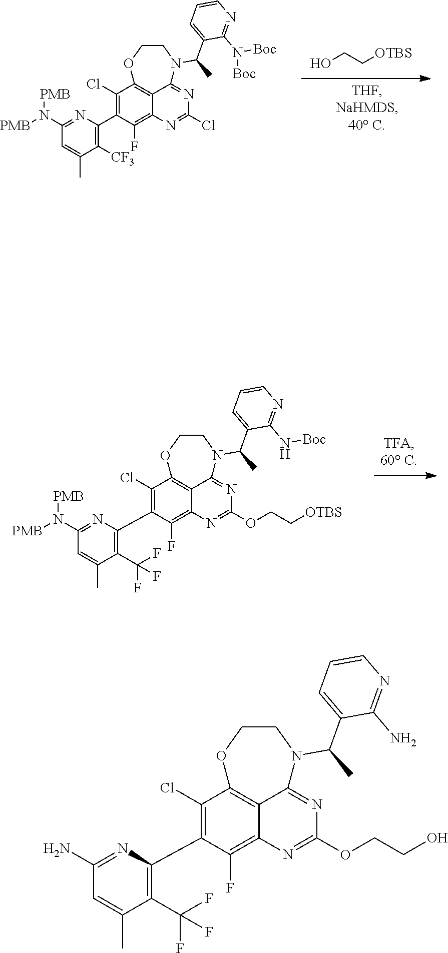

Step 1: tert-butyl (3-((1R)-1-(9-(6-(bis(4-methoxybenzyl)amino)-4-methyl-3-(trifluoromethyl)pyridin-2-yl)-2-(2-((tert-butyldimethylsilyl)oxy)ethoxy)-8-chloro-10-fluoro-5,6-dihydro-4H-[1,4]oxazepino[5,6,7-de]quinazolin-4-yl)ethyl)pyridin-2-yl)carbamate

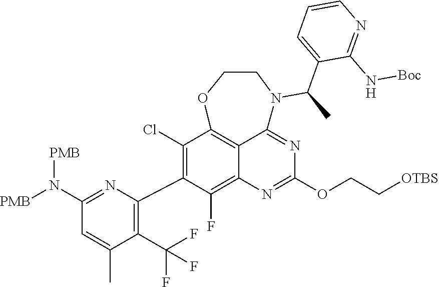

2-((tert-butyldimethylsilyl)oxy)ethan-1-ol (65 mg, 0.36 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (125 mg, 0.124 mmol) to give the crude title compound (129 mg) as a tan oil which. LC-MS: (ESI, m/z): 1047.9 [M+H]⁺

Step 2: 2-(((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)ethan-1-ol

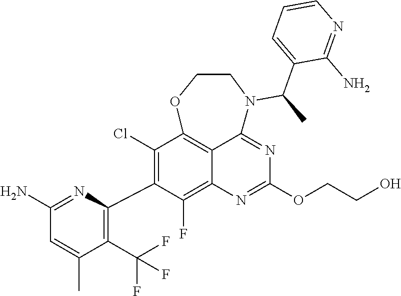

A tert-butyl (3-((1R)-1-(9-(6-(bis(4-methoxybenzyl)amino)-4-methyl-3-(trifluoromethyl)pyridin-2-yl)-2-(2-((tert-butyldimethylsilyl)oxy)ethoxy)-8-chloro-10-fluoro-5,6-dihydro-4H-[1,4]oxazepino[5,6,7-de]quinazolin-4-yl)ethyl)pyridin-2-yl)carbamate

(100.0 mg, 0.095 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Agilent 1290 Infinity II Autoscale Prep LC/MS with the following conditions: Column: Triart C18, 50*30 mm, 5 μm; Sample Solvent: DMSO: Mobile Phase A: water (0.1% Formic acid in Water), Mobile Phase B: acetonitrile; Flow rate: 60 mL/min; Gradient: 5% B to 50% B in 10 min; Wave Length: 254 nm; Column Temp: 25° C.; Agilent 10-min LCMS method retention time (min): 3.24 (undesired atropisomer 4.66) to afford 2-(((R)-9-(6-amino-4-methyl-3-(trifluoromethyl)pyridin-2-yl)-4-((R)-1-(2-aminopyridin-3-yl)ethyl)-8-chloro-10-fluoro-5,6-dihydro-4H-[1,4]oxazepino[5,6,7-de]quinazolin-2-yl)oxy)ethan-1-ol (14.8 mg, 0.025 mmol, 26.1% yield). LC-MS: (ESI, m/z): 594.1 [M+H]⁺

Example 156: ¹H NMR (500 MHz, DMSO-d₆, ppm) δ 7.97 (d, J=5.0 Hz, 1H), 7.63 (d, J=7.6 Hz, 1H), 6.79 (s, 2H), 6.67 (dd, J=7.6, 4.8 Hz, 1H), 6.47 (s, 1H), 6.27 (q, J=6.9 Hz, 1H), 5.63 (s, 2H), 4.89 (t, J=5.6 Hz, 1H), 4.45-4.32 (m, 3H), 4.27-4.21 (m, 1H), 3.74 (q, J=5.3 Hz, 2H), 3.66-3.58 (m, 1H), 3.40-3.32 (m, 2H), 2.36 (s, 3H), 1.56 (d, J=6.7 Hz, 3H). LC-MS: (ESI, m/z): 594.1 [M+H]⁺

Example 157: 6-((R)-2-((2-oxabicyclo[2.2.2]octan-4-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

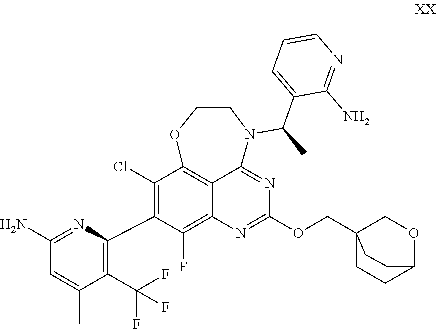

### Synthetic Route

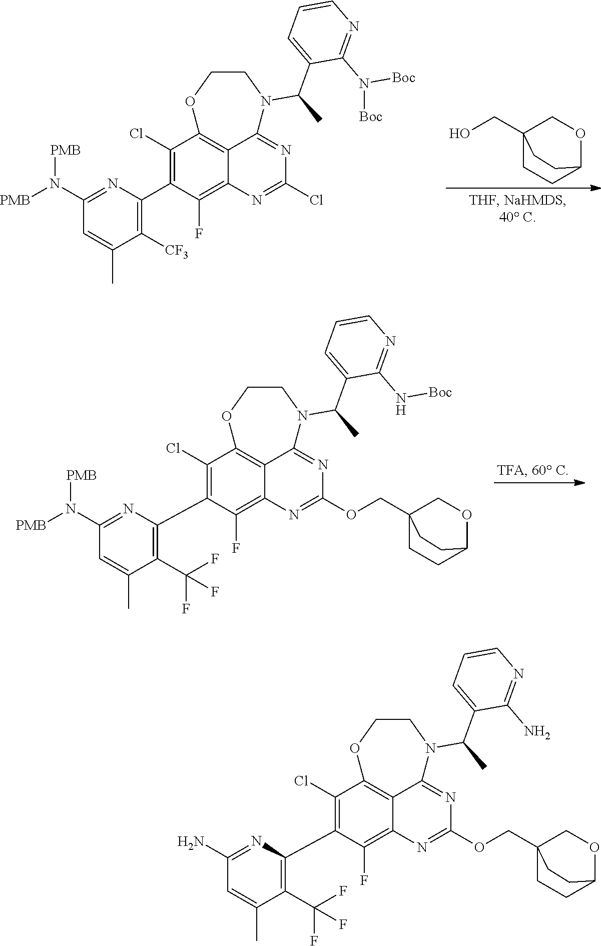

Step 1: tell-butyl (3-((1R)-1-(2-((2-oxabicyclo[2.2.2]octan-4-yl)methoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate

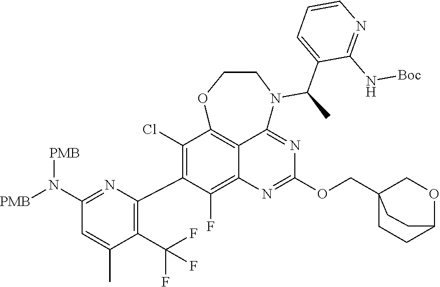

(2-oxabicyclo[2.2.2]octan-4-yl)methanol (63 mg, 0.443 mmol) was reacted with Di-tert-butyl(3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)pyridin-2-yl)iminocarboxylate (150 mg, 0.149 mmol) to give the crude title compound (131 mg) as a tan oil which. LC-MS: (ESI, m/z): 1013.9 [M+H]⁺

Step 2: 6-((R)-2-((2-oxabicyclo[2.2.2]octan-4-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine

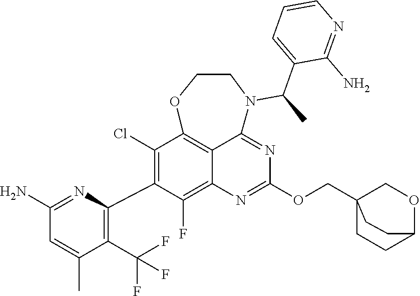

A tert-butyl (3-((1R)-1-(2-((2-oxabicyclo[2.2.2]octan-4-yl)methoxy)-9-(6-(bis(4-methoxybenzyl)amino)-4-methyl-3-(trifluoromethyl)pyridin-2-yl)-8-chloro-10-fluoro-5,6-dihydro-4H-[1,4]oxazepino[5,6,7-de]quinazolin-4-yl)ethyl)pyridin-2-yl)carbamate (100 mg, 0.099 mmol) in trifluoroacetic acid (2 mL) was stirred for 5 hours at 55° C. After completion, the reaction mixture was adjusted to pH >7 with sodium bicarbonate saturated solution, extracted with ethyl acetate and the organic layer was combined. The organic layer was dried over anhydrous sodium sulfate and concentrated under vacuum. The product was purified by Interchim PuriFlash 4250 HPLC with the following conditions: Column: XSelect CSH Prep C18, 50*30 mm, 5 μm; Sample Solvent: DMSO: Mobile Phase A: water (0.1% Ammonium Hydroxide in Water), Mobile Phase B: acetonitrile; Flow rate: 60 mL/min; Gradient: 30% B to 70% B in 10 min; Wave Length: 240 nm; Column Temp: 25° C.: Agilent 10-min LCMS method retention time (min): 3.82 (undesired atropisomer 4.31) to afford 6-((R)-2-((2-oxabicyclo[2.2.2]octan-4-yl)methoxy)-4-((R)-1-(2-aminopyridin-3-yl)ethyl)-8-chloro-10-fluoro-5,6-dihydro-4H-[1,4]oxazepino[5,6,7-de]quinazolin-9-yl)-4-methyl-5-(trifluoromethyl)pyridin-2-amine (27.32 mg, 0.041 mmol, 41.1% yield). LC-MS: (ESI, m/z): 674.2 [M+H]⁺

Example 157: ¹H NMR (400 MHz, DMSO-d₆, ppm) δ 7.96 (dd, J=4.9, 1.8 Hz, 1H), 7.61 (d, J=7.5 Hz, 1H), 6.79 (s, 2H), 6.65 (dd, J=7.4, 4.9 Hz, 1H), 6.47 (s, 1H), 6.21 (q, J=7.0 Hz, 1H), 5.73 (s, 2H), 4.48-4.39 (m, 1H), 4.31-4.22 (m, 1H), 4.04 (s, 2H), 3.72-3.61 (m, 4H), 3.44-3.35 (m, 1H), 2.35 (s, 3H), 1.98-1.84 (m, 2H), 1.69-1.51 (m, 9H). LC-MS: (ESI, m/z): 674.2 [M+H]⁺

Example 158a and 158b: 3-((R)-1-((R)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrazin-2-amine& 3-((R)-1-((S)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrazin-2-amine

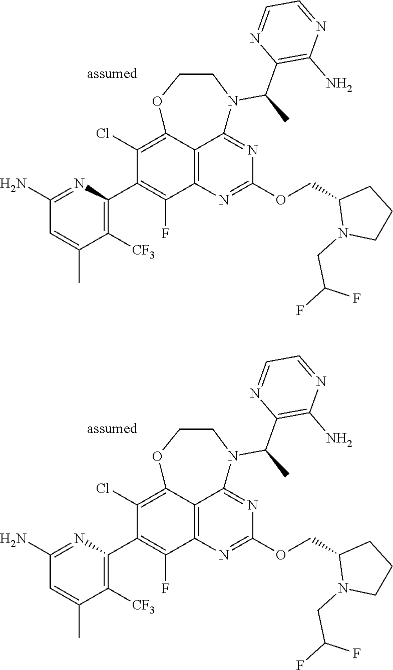

### Synthetic Route

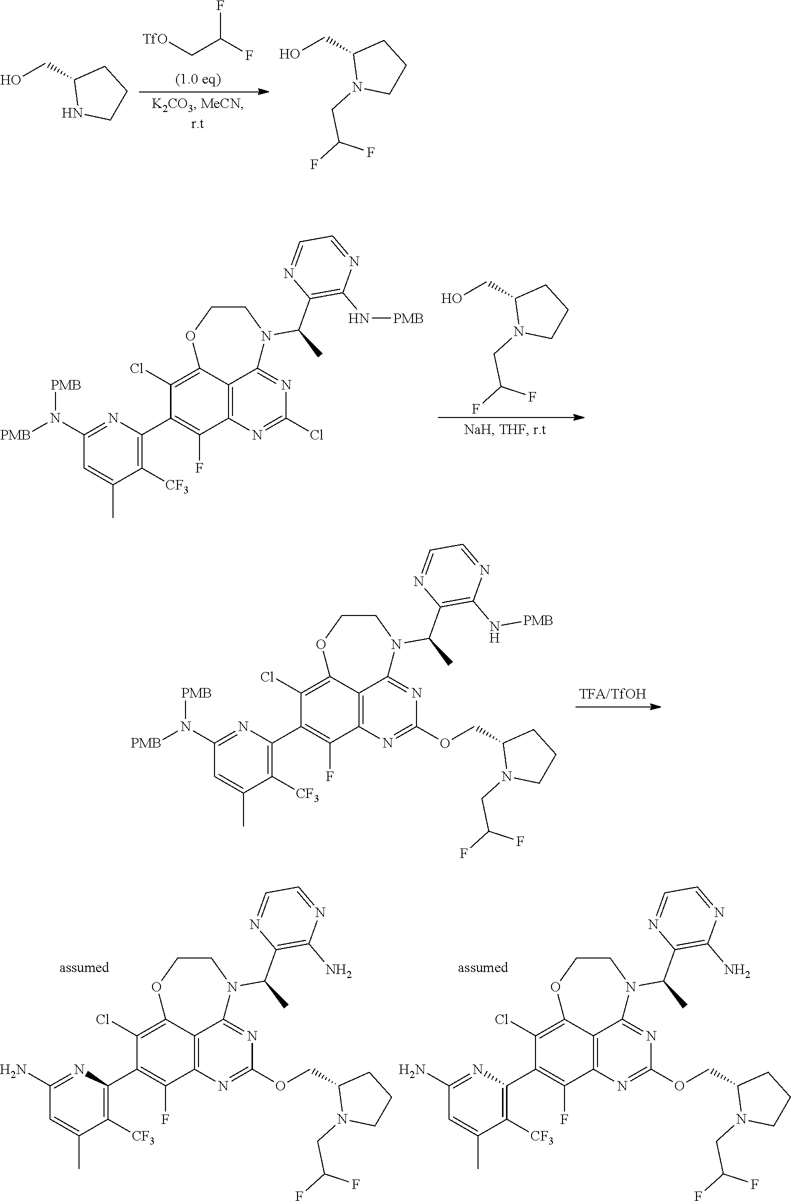

Step 1: (S)-(1-(2,2-difluoroethyl)pyrrolidin-2-yl)methanol

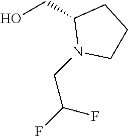 [SMILES: OC[C@@H]1CCCN1CC(F)F]

A solution of I-prolinol (4.8 mL, 49.4 mmol), 2,2-difluoroethyl trifluoromethanesulfonate (12.70 g, 59.3 mmol) and potassium carbonate (20.50 g, 148.3 mmol) in acetone (50 mL) was stirred at 25° C. for 8 hours. After completion, the reaction mixture was filtered. The filter was concentrated under vacuum. The residue was purified by flash chromatography on silica gel eluting with methanol/dichloromethane (1:8) to afford (S)-(1-(2,2-difluoroethyl)pyrrolidin-2-yl)methanol (6.00 g, 36.3 mmol, 73.5% yield) as a yellow oil. LC-MS: (ESI, m/z): 166.1 [M−H]⁺

Step 2: 3-((1R)-1-(9-(6-(bis(4-methoxybenzyl)amino)-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)-N-(4-methoxybenzyl)pyrazin-2-amine

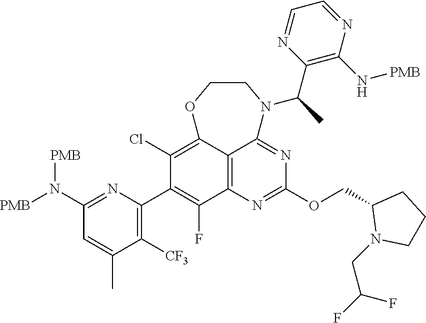

A solution of (S)-(1-(2,2-difluoroethyl)pyrrolidin-2-yl)methanol (142.1 mg, 0.8 mmol) in tetrahydrofuran (4 mL) was added sodium hydride (30.9 mg, 1.3 mmol, 60% purity) and stirred at 0° C. for 15 minutes. Then 3-[(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5(14),6,8-pentaen-13-yl]ethyl]-N-[(4-methoxyphenyl)methyl]pyrazin-2-amine (400.0 mg, 0.4 mmol) was added and stirred at 25° C. for 2 hours. After completion, the reaction was quenched with saturated ammonium chloride solution, diluted with water, extracted with ethyl acetate. The organic layers were combined, washed with water, dried over anhydrous sodium sulfate and concentrated under vacuum. The residue was purified by flash chromatography on silica gel eluting with methanol/dichloromethane (1:8) to afford 3-((1R)-1-(9-(6-(bis(4-methoxybenzyl)amino)-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (390.0 mg, 0.4 mmol, 85.6% yield) as a yellow solid.

Step 3: 3-((R)-1-((R)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrazin-2-amine

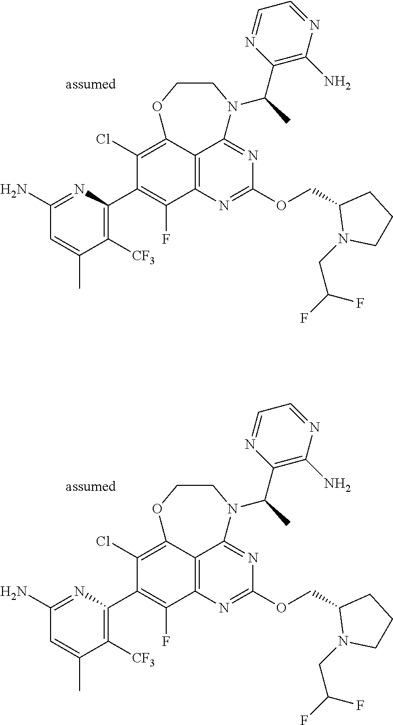

A solution of 3-[(1R)-1-[7-[6-[bis[(4-methoxyphenyl)methyl]amino]-4-methyl-3-(trifluoromethyl)-2-pyridyl]-8-chloro-3-[[(2S)-1-(2,2-difluoroethyl)pyrrolidin-2-yl]methoxy]-6-fluoro-10-oxa-2,4,13-triazatricyclo[7.4.1.05,14]tetradeca-1,3,5(14),6,8-pentaen-13-yl]ethyl]-N-[(4-methoxyphenyl)methyl]pyrazin-2-amine (300.0 mg, 0.28 mmol) in trifluoroacetic acid (3 mL) and trifluoromethanesulfonic acid (0.3 mL) was stirred at 25° C. for 1 hour. After completion, the reaction mixture was concentrated under reduced pressure. The residue was diluted with dichloromethane, adjusted to pH=7 with saturated sodium bicarbonate solution. The resulting solution was extracted with dichloromethane, dried over anhydrous sodium sulfate and concentrated under vacuum. The residue was purified by flash chromatography on silica gel eluting with dichloromethane/methanol (10:1) to afford crude product. The crude product was further purified by Prep-HPLC with the following conditions (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 46% B to 65% B in 9.5 min, 65% B: Wave Length: 254/220 nm) to afford 3-((R)-1-((R)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrazin-2-amine (39.6 mg, 0.06 mmol, 19.7% yield) and 3-((R)-1-((S)-9-(6-amino-4-methyl-3-(trifluoromethyl)pyridin-2-yl)-8-chloro-2-(((S)-1-(2,2-difluoroethyl)pyrrolidin-2-yl)methoxy)-10-fluoro-5,6-dihydro-4H-[1,4]oxazepino[5,6,7-de]quinazolin-4-yl)ethyl)pyrazin-2-amine (30.6 mg, 0.04 mmol, 15.4% yield).

Example 158a: ¹H NMR (300 MHz, DMSO-d₆, ppm) δ 7.96 (d, J=2.7 Hz, 1H), 7.80 (d, J=2.7 Hz, 1H), 6.81 (s, 2H), 6.49 (s, 1H), 6.39-5.85 (m, 4H), 4.53 (dd, J=12.4, 6.1 Hz, 1H), 4.42-4.24 (m, 2H), 4.14 (dd, J=10.8, 6.7 Hz, 1H), 3.84 (dd, J=15.3, 6.3 Hz, 1H), 3.66-3.52 (m, 1H), 3.41-3.22 (m, 1H), 3.15-3.06 (m, 1H), 3.05-2.98 (m, 1H), 2.95-2.71 (m, 1H), 2.48-2.41 (m, 4H), 2.00-1.85 (m, 1H), 1.80-1.62 (m, 3H), 1.58 (d, J=6.8 Hz, 3H). LCMS (ESI, m/z): 698.1 [M+H]⁺.

Example 158b: ¹H NMR (300 MHz, DMSO-d₆, ppm) δ 7.95 (d, J=2.7 Hz, 1H), 7.78 (d, J=2.7 Hz, 1H), 6.82 (s, 2H), 6.48 (s, 1H), 6.42 (s, 2H), 6.33-5.86 (m, 2H), 4.70-4.55 (m, 1H), 4.43-4.32 (m, 1H), 4.26 (dd, J=10.8, 5.1 Hz, 1H), 4.15 (dd, J=10.8, 6.7 Hz, 1H), 3.99-3.85 (m, 1H), 3.70-3.57 (m, 1H), 3.38-3.20 (m, 1H), 3.16-2.96 (m, 2H), 2.90-2.69 (m, 1H), 2.48-2.33 (m, 4H), 2.09-1.85 (m, 1H), 1.81-1.70 (m, 2H), 1.69-1.53 (m, 4H). LCMS (ESI, m/z): 698.1 [M+H]⁺.

Example 159a & 159b: 3-((R)-1-((R)-9-(6-amino-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)pyrazin-2-amine and 3-((R)-1-((S)-9-(6-amino-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)pyrazin-2-amine

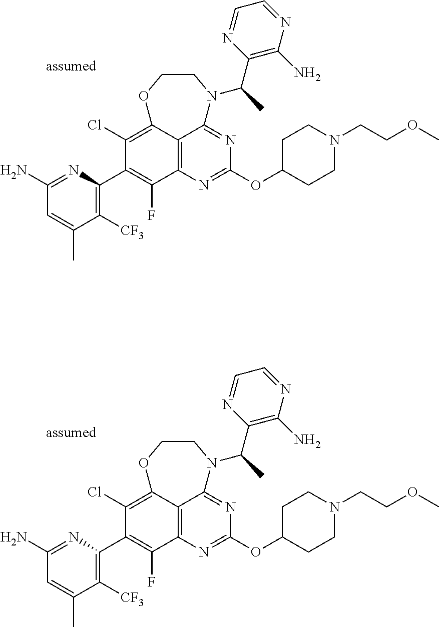

### Synthetic Route:

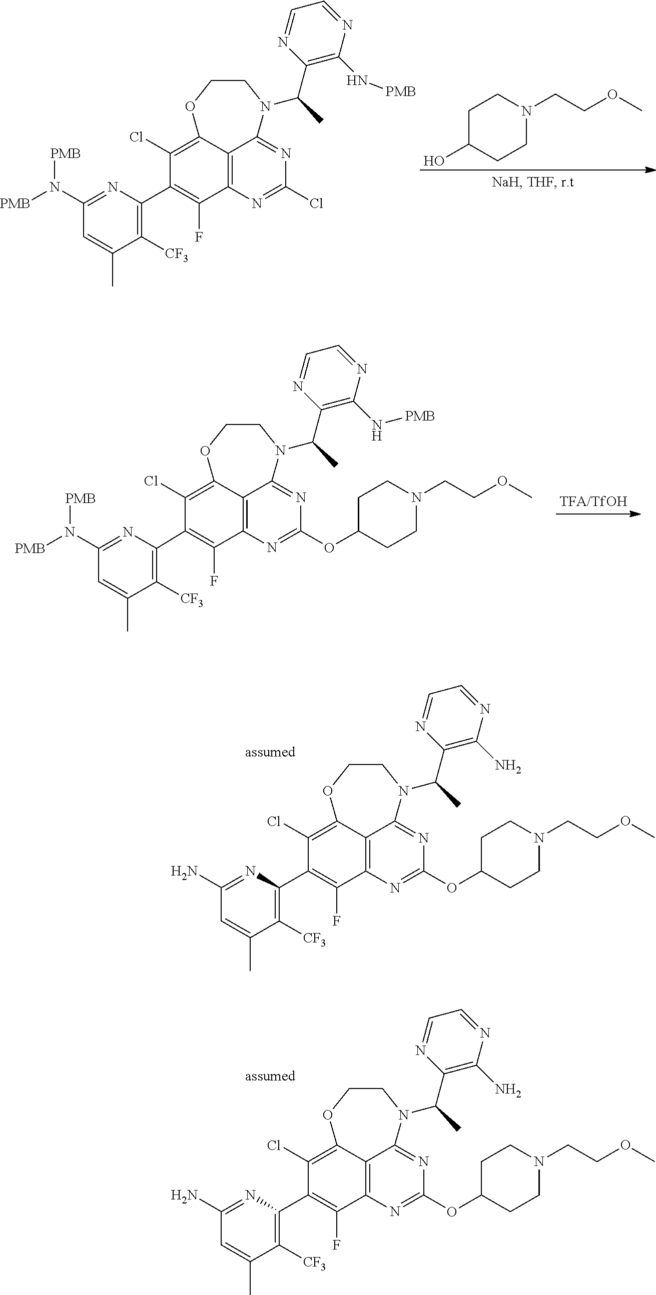

Step 1: 3-((1R)-1-(9-(6-(bis(4-methoxybenzyl)amino)-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)-N-(4-methoxybenzyl)pyrazin-2-amine

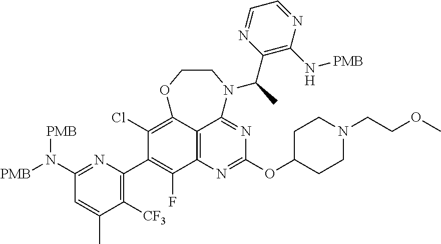

A solution of 1-(2-methoxyethyl)piperidin-4-ol (128.0 mg, 0.80 mmol) in tetrahydrofuran (3 mL) was added sodium hydride (75.0 mg, 1.88 mmol in 60% dispersion in mineral oil) was stirred at 0° C. for 0.5 hours. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (250.0 mg, 0.27 mmol) was added and stirred at 25° C. for 1 hour. After completion, the reaction was quenched with saturated ammonium chloride solution. The reaction mixture was diluted with water. The resulted solution was extracted with ethyl acetate and washed with brine. The residue was purified by flash chromatography on silica gel eluting with methylene chloride/methanol (3:1) to afford 3-((1R)-1-(9-(6-(bis(4-methoxybenzyl)amino)-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)-N-(4-methoxybenzyl)pyrazin-2-amine (90.0 mg, 0.085 mmol, 31.8% yield) as a yellow solid. LC-MS: (ESI, m/z): 1152.5 [M+H]⁺

Step 2: 3-((R)-1-((R)-9-(6-amino-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)pyrazin-2-amine and 3-((R)-1-((S)-9-(6-amino-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)pyrazin-2-amine

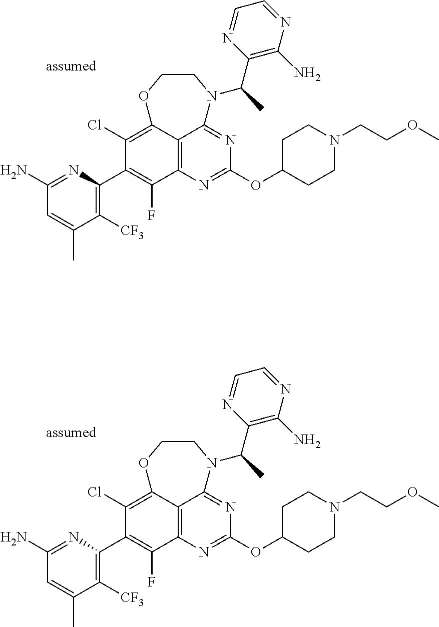

A solution of 3-((1R)-1-(9-(6-(bis(4-methoxybenzyl)amino)-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)-N-(4-methoxybenzyl)pyrazin-2-amine (90.0 mg, 0.09 mmol) in trifluoroacetic acid (1 mL) and trifluoromethanesulfonic acid (0.1 mL) was stirred at 25° C. for 0.5 hour. After completion, the solvent was concentrated under vacuum. The product was purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min: Gradient: 17% B to 42% B in 9 min, 42% B; Wave Length: 254/220 nm; RT1(min): 8.9 to afford 3-((R)-1-((R)-9-(6-amino-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)pyrazin-2-amine (6.8 mg, 0.0098 mmol, 11.5% yield) and 3-((R)-1-((S)-9-(6-amino-4-methyl-3-(trifluoromethyl)pyridin-2-yl)-8-chloro-10-fluoro-2-((1-(2-methoxyethyl)piperidin-4-yl)oxy)-5,6-dihydro-4H-[1,4]oxazepino[5,6,7-de]quinazolin-4-yl)ethyl)pyrazin-2-amine (5.1 mg, 0.0074 mmol, 8.6% yield).

Example 159a: ¹H NMR (300 MHz, DMSO-da, ppm) δ 7.94 (d, J=2.7 Hz, 1H), 7.76 (d, J=2.7 Hz, 1H), 6.81 (s, 2H), 6.48 (s, 1H), 6.36 (s, 2H), 6.22 (q, J=6.7 Hz, 1H), 5.07-4.93 (m, 1H), 4.55 (dd, J=11.9, 6.9 Hz, 1H), 4.39 (dd, J=11.9, 6.2 Hz, 1H), 3.87 (dd, J=15.5, 6.2 Hz, 1H), 3.62 (dd, J=15.6, 6.9 Hz, 1H), 3.43 (t, J=5.9 Hz, 2H), 3.31-3.30 (m, 1H), 3.24 (s, 3H), 2.81-2.59 (m, 2H), 2.49-2.47 (m, 1H), 2.37 (d, J=1.6 Hz, 3H), 2.32-2.17 (m, 2H), 2.05-1.90 (m, 1H), 1.87-1.75 (m, 1H), 1.74-1.63 (m, 1H), 1.62-1.46 (m, 4H). LC-MS: (ESI, m/z): 692.1 [M+H]⁺

Example 159b: ¹H NMR (300 MHz, DMSO-d₆, ppm) δ 7.94 (d, J=2.7 Hz, 1H), 7.76 (d, J=2.7 Hz, 1H), 6.81 (s, 2H), 6.48 (s, 1H), 6.36 (s, 2H), 6.22 (q, J=6.7 Hz, 1H), 5.07-4.93 (m, 1H), 4.55 (dd, J=11.9, 6.9 Hz, 1H), 4.39 (dd, J=11.9, 6.2 Hz, 1H), 3.87 (dd, J=15.5, 6.2 Hz, 1H), 3.62 (dd, J=15.6, 6.9 Hz, 1H), 3.43 (t, J=5.9 Hz, 2H), 3.31-3.30 (m, 1H), 3.24 (s, 3H), 2.81-2.59 (m, 2H), 2.49-2.47 (m, 1H), 2.37 (d, J=1.6 Hz, 3H), 2.32-2.17 (m, 2H), 2.05-1.90 (m, 1H), 1.87-1.75 (m, 1H), 1.74-1.63 (m, 1H), 1.62-1.46 (m, 4H). LC-MS: (ESI, m/z): 692.1 [M+H]⁺

Example 160a & 160b: 3-((R)-1-((R)-9-(6-amino-5-fluoro-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine and 3-((R)-1-((S)-9-(6-amino-5-fluoro-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine

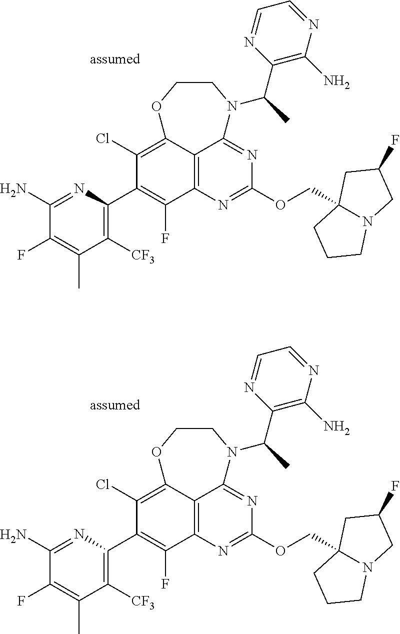

### Synthetic Route:

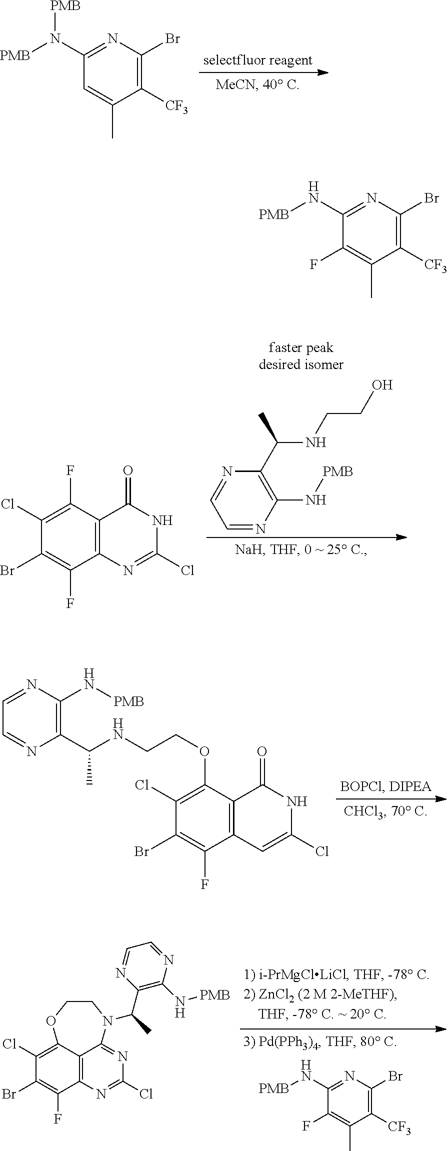

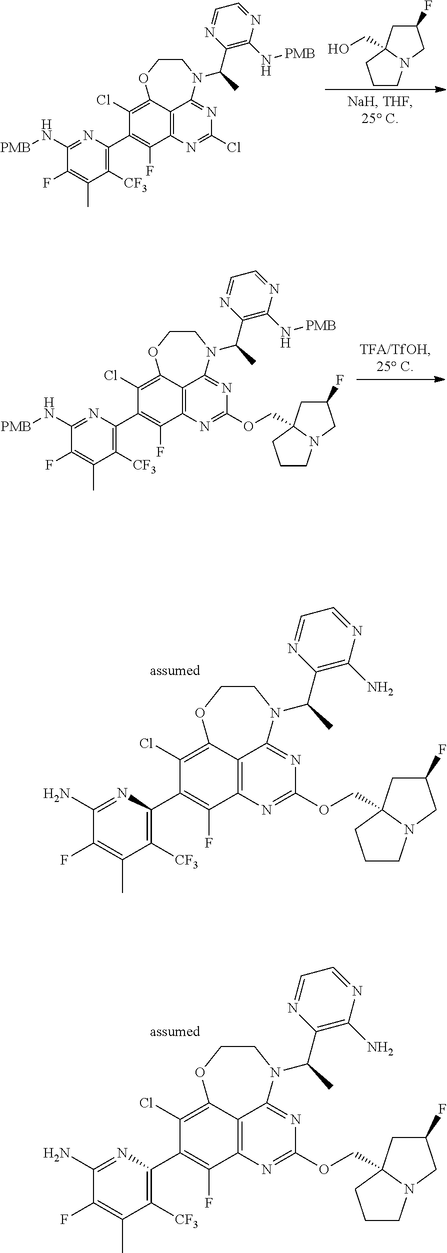

Step 1: 6-bromo-3-fluoro-N-(4-methoxybenzyl)-4-methyl-5-(trifluoromethyl)pyridin-2-amine

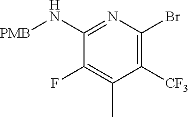

A solution of 6-bromo-N,N-bis(4-methoxybenzyl)-4-methyl-5-(trifluoromethyl)pyridin-2-amine (8.0 g, 16.15 mmol) in acetonitrile (150 mL) was added 1-chloromethyl-4-fluoro-1,4-diazoniabicyclo[2.2.2]octanebis(tetrafluoroborate) (1.14 g, 32.3 mmol) and stirred at 40° C. for 17 hours. After completion, the reaction was quenched with saturated sodium metabisulfite solution. The reaction mixture was diluted with dichloromethane, washed with water and the organic layer was combined. The organic layer was dried over anhydrous sodium sulfate and concentrated under vacuum. The residue was purified by flash chromatography on reverse-phase column eluting with water/acetonitrile (3:2) to afford 6-bromo-3-fluoro-N-(4-methoxybenzyl)-4-methyl-5-(trifluoromethyl)pyridin-2-amine (750 mg, 1.6405 mmol, 10.2% yield) as an orange solid. LC-MS: (ESI, m/z): 393.1 [M+H]⁺

Step 2: (R)-7-bromo-2,6-dichloro-8-fluoro-5-(2-((1-(3-((4-methoxybenzyl)amino)pyrazin-2-yl)ethyl)amino)ethoxy)quinazolin-4(3H)-one

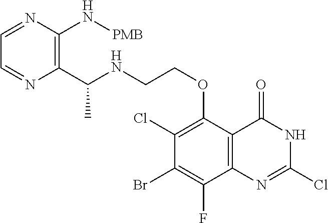

Similar to as described in General Procedure A. A solution of (R)-2-((1-(3-((4-methoxybenzyl)amino)pyrazin-2-yl)ethyl)amino)ethan-1-ol (1.44 g, 4.77 mmol) in tetrahydrofuran (15 mL) was added sodium hydride (0.33 g, 13.65 mmol, 60% dispersion in mineral oil) and stirred at 0° C. for 15 minutes. Then the solution was added into a solution of 7-bromo-2,6-dichloro-5,8-difluoroquinazolin-4(3H)-one (1.5 g, 4.55 mmol) in tetrahydrofuran (15 mL) and stirred at 0° C. for 2 hours. After completion, the reaction was quenched with saturated ammonium chloride solution. The reaction mixture was diluted with ethyl acetate. The resulting solution was extracted with water and washed with brine. The organic layer was dried over anhydrous sodium sulfate and concentrated under vacuum. The residue was purified by flash chromatography on silica gel eluting with methanol/dichloromethane (1:25) to afford (R)-7-bromo-2,6-dichloro-8-fluoro-5-(2-((1-(3-((4-methoxybenzyl)amino)pyrazin-2-yl)ethyl)amino)ethoxy)quinazolin-4(3H)-one (1.10 g, 1.80 mmol, 39.5% yield) as a yellow solid. LC-MS: (ESI, m/z): 612.2 [M+H]⁺

Step 3: (R)-3-(1-(9-bromo-2,8-dichloro-10-fluoro-5,6-dihydro-4H-[1,4]oxazepino[5,6,7-de]quinazolin-4-yl)ethyl)-N-(4-methoxybenzyl)pyrazin-2-amine

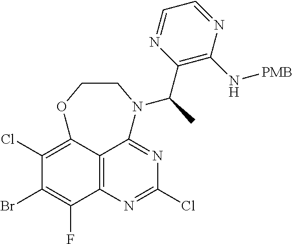

Similar to as described in General Procedure B. A solution of (R)-7-bromo-2,6-dichloro-8-fluoro-5-(2-((1-(3-((4-methoxybenzyl)amino)pyrazin-2-yl)ethyl)amino)ethoxy)quinazolin-4(3H)-one (1.24 g, 2.03 mmol) in chloroform (15 mL) was added bis(2-oxo-3-oxazolidinyl)phosphinic chloride (1.03 g, 4.05 mmol) and N,N-diisopropylethylamine (1.06 mL, 6.08 mmol) and stirred at 70° C. for 2 hours. After completion, the reaction mixture was diluted with water. The resulting solution was extracted with ethyl acetate and washed with brine. The organic layer was dried over anhydrous sodium sulfate and concentrated under vacuum. The residue was purified by flash chromatography on silica gel eluting with ethyl acetate/petroleum ether (3:2) to afford (R)-3-(1-(9-bromo-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (800.0 mg, 1.35 mmol, 66.5% yield) as a yellow solid. LC-MS: (ESI, m/z): 594.2 [M+H]⁺

Step 4: 3-((1R)-1-(2,8-dichloro-10-fluoro-9-(5-fluoro-6-((4-methoxybenzyl)amino)-4-methyl-3-(trifluoromethyl)pyridin-2-yl)-5,6-dihydro-4H-[1,4]oxazepino[5,6,7-de]quinazolin-4-yl)ethyl)-N-(4-methoxybenzyl)pyrazin-2-amine

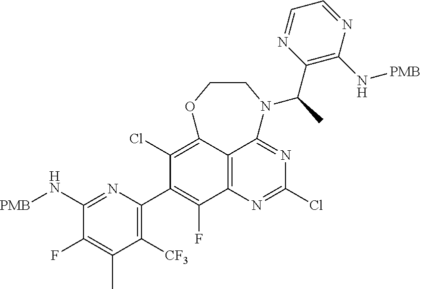

Under nitrogen, a solution of (R)-3-(1-(9-bromo-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (300.0 mg, 0.5 mmol) in tetrahydrofuran (3.0 mL) was stirred at −78° C. Then isopropyl magnesium chloride lithium chloride (0.51 mL, 0.66 mmol, 1.3 M in tetrahydrofuran) was added and stirred at −78° C. for 30 minutes. Then zinc chloride (0.76 mL, 1.51 mmol, 2 M in 2-methyltetrahydrofuran) was added at −78° C. and stirred at 25° C. for 30 minutes. Under nitrogen, the solution was added into the solution of 6-bromo-3-fluoro-N-(4-methoxybenzyl)-4-methyl-5-(trifluoromethyl)pyridin-2-amine (216.0 mg, 0.55 mmol) and tetrakis(triphenylphosphine)palladium (90.0 mg, 0.08 mmol) in tetrahydrofuran (2 mL) at 25° C. The resulting solution was stirred at 80° C. for 1 hour. After completion, the reaction was quenched with water. The reaction mixture was diluted with water. The resulting solution was extracted with ethyl acetate and washed with brine. The organic layer was dried over anhydrous sodium sulfate and concentrated under vacuum. The residue was purified by flash chromatography on C18 gel eluting with acetonitrile/water (6:1) to afford 3-((1R)-1-(2,8-dichloro-10-fluoro-9-(5-fluoro-6-((4-methoxybenzyl)amino)-4-methyl-3-(trifluoromethyl)pyridin-2-yl)-5,6-dihydro-4H-[1,4]oxazepino[5,6,7-de]quinazolin-4-yl)ethyl)-N-(4-methoxybenzyl)pyrazin-2-amine (100.0 mg, 0.12 mmol, 23.9% yield) as a yellow solid. LC-MS: (ESI, m/z): 827.6 [M+H]⁺

Step 5: 3-((1R)-1-(8-chloro-10-fluoro-9-(5-fluoro-6-((4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine

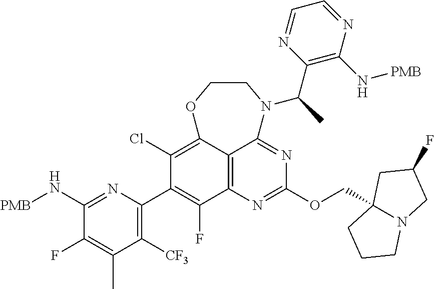

A solution of ((2R,7aS)-2-fluorotetrahydro-1H-pyrrolizin-7a(5H)-yl)methanol (40.0 mg, 0.2500 mmol) in tetrahydrofuran (1.0 mL) was added sodium hydride (25.0 mg, 0.63 mmol, 60% dispersion in mineral oil) and stirred at 25° C. for 30 minutes. Then 3-((1R)-1-(2,8-dichloro-10-fluoro-9-(5-fluoro-6-((4-methoxybenzyl)amino)-4-methyl-3-(trifluoromethyl)pyridin-2-yl)-5,6-dihydro-4H-[1,4]oxazepino[5,6,7-de]quinazolin-4-yl)ethyl)-N-(4-methoxybenzyl)pyrazin-2-amine (100.0 mg, 0.1200 mmol) was added and stirred at 25° C. for 1 hour. After completion, the reaction was quenched with saturated ammonium chloride solution. The reaction mixture was diluted with water. The resulting solution was extracted with ethyl acetate and washed with brine. The organic layer was dried over anhydrous sodium sulfate and concentrated under vacuum. The residue was purified by flash chromatography on silica gel eluting with dichloromethane/methanol (9:1) to afford 3-((1R)-1-(8-chloro-10-fluoro-9-(5-fluoro-6-((4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine (80.0 mg, 0.08 mmol, 69.7% yield) as a yellow solid. LC-MS: (ESI, m/z): 950.3 [M+H]⁺

Step 6: 3-((R)-1-((R)-9-(6-amino-5-fluoro-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine and 3-((R)-1-((S)-9-(6-amino-5-fluoro-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine

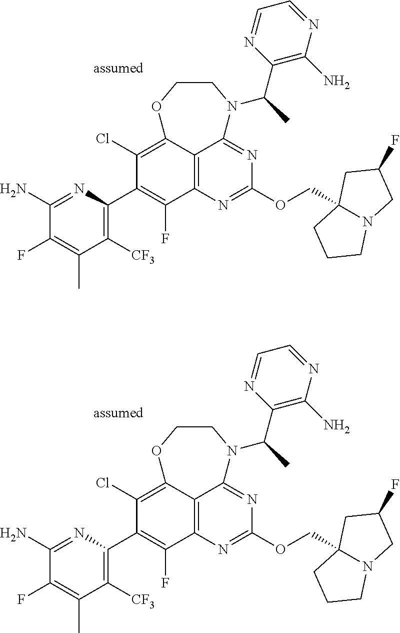

A solution of 3-((1R)-1-(8-chloro-10-fluoro-9-(5-fluoro-6-((4-methoxybenzyl)amino)-4-methyl-3-(trifluoromethyl)pyridin-2-yl)-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)-N-(4-methoxybenzyl)pyrazin-2-amine (60.0 mg, 0.06 mmol) in trifluoroacetic acid (1.0 mL) and trifluoromethanesulfonic (0.1 mL) was stirred at 25° C. for 30 minutes. After completion, the solvent was concentrated under vacuum. The product was purified by Prep-HPLC with the following conditions: Column: XSelect CSH Fluoro Phenyl, 30*150 mm, 5 μm; Mobile Phase A: Water (10 mmol/L NH4HCO3), Mobile Phase B: ACN; Flow rate: 60 mL/min; Gradient: 44% B to 64% B in 10 min, 64% B; Wave Length: 254/220 nm; RT1(min): 7.35 to afford 3-((R)-1-((R)-9-(6-amino-5-fluoro-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine (2.7 mg, 0.004 mmol, 6% yield) and 3-((R)-1-((S)-9-(6-amino-5-fluoro-4-methyl-3-(trifluoromethyl)pyridin-2-yl)-8-chloro-10-fluoro-2-(((2R,7aS)-2-fluorotetrahydro-1H-pyrrolizin-7a(5H)-yl)methoxy)-5,6-dihydro-4H-[1,4]oxazepino[5,6,7-de]quinazolin-4-yl)ethyl)pyrazin-2-amine (7.4 mg, 0.01 mmol, 16.5% yield).

Example 160a: ¹H NMR (300 MHz, DMSO-da, ppm) δ 7.95 (d, J=2.7 Hz, 1H), 7.78 (d, J=2.7 Hz, 1H), 7.19 (s, 2H), 6.40 (s, 2H), 6.28 (q, J=6.8 Hz, 1H), 5.28 (d, J=54.3 Hz, 1H), 4.55 (dd, J=12.2, 6.4 Hz, 1H), 4.37 (dd, J=11.9, 6.3 Hz, 1H), 4.04 (s, 2H), 3.87 (dd, J=15.8, 6.4 Hz, 1H), 3.63 (dd, J=15.5, 6.7 Hz, 1H), 3.08 (d, J=7.6 Hz, 2H), 2.98 (s, 1H), 2.82 (dd, J=15.2, 8.3 Hz, 1H), 2.35 (s, 3H), 2.16-2.08 (m, 1H), 2.06-1.92 (m, 2H), 1.89-1.68 (m, 3H), 1.58 (d, J=6.8 Hz, 3H). LC-MS: (ESI, m/z): 710.2 [M+H]⁺

Example 160b: ¹H NMR (300 MHz, DMSO-d₆, ppm) δ 7.94 (d, J=2.6 Hz, 1H), 7.77 (d, J=2.7 Hz, 1H), 7.20 (s, 2H), 6.49 (s, 2H), 6.26 (q, J=6.7 Hz, 1H), 5.28 (d, J=54.4 Hz, 1H), 4.63 (dd, J=11.7, 6.9 Hz, 1H), 4.40 (dd, J=11.9, 6.1 Hz, 1H), 4.10-3.89 (m, 3H), 3.67 (dd, J=15.6, 7.0 Hz, 1H), 3.15-3.03 (m, 2H), 2.98 (s, 1H), 2.87-2.76 (m, 1H), 2.35 (s, 3H), 2.19-2.08 (m, 1H), 2.07-1.92 (m, 2H), 1.89-1.71 (m, 3H), 1.60 (d, J=6.8 Hz, 3H). LC-MS: (ESI, m/z): 710.2 [M+H]⁺

Example 161a & 161b: 5-((R)-1-((R)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrimidin-4-amine

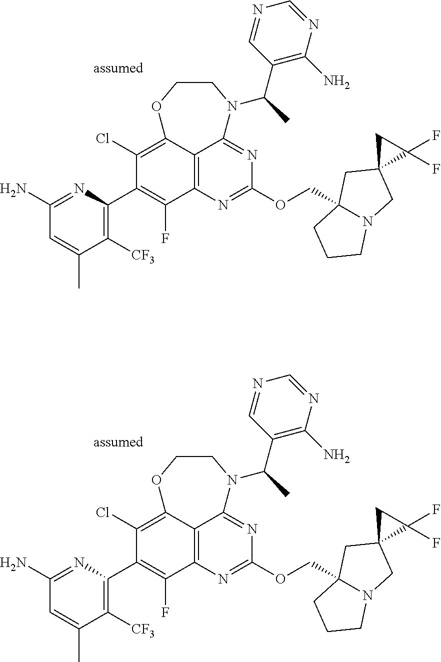

### Synthetic Route

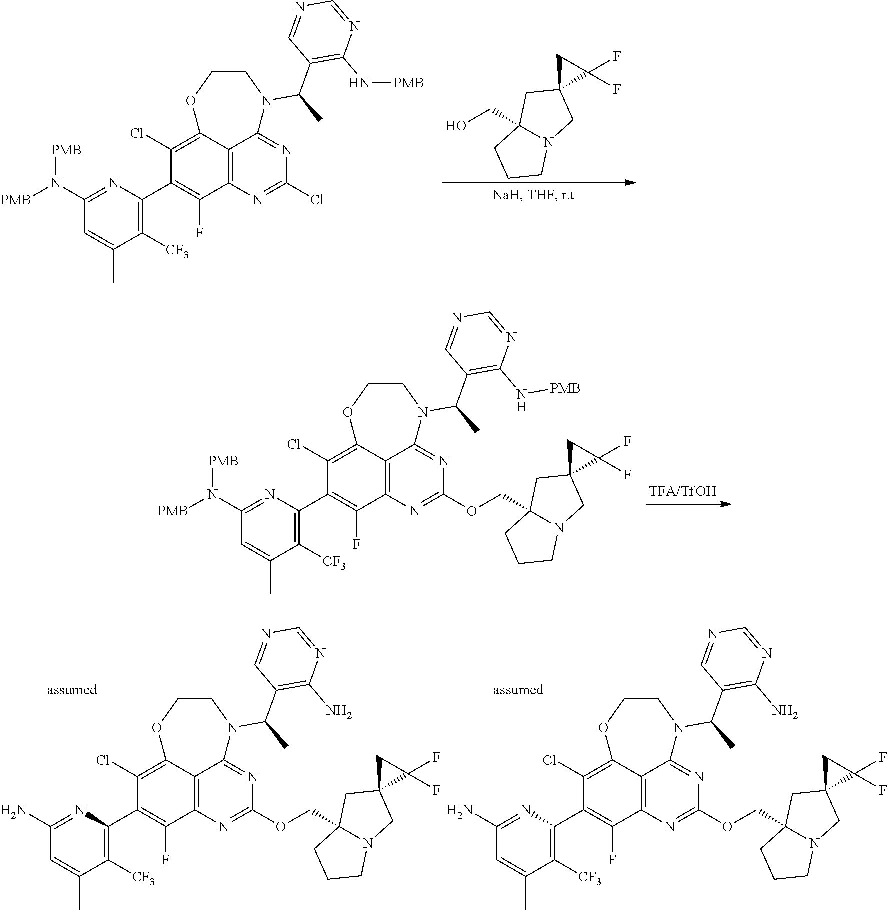

Step 1: 5-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine

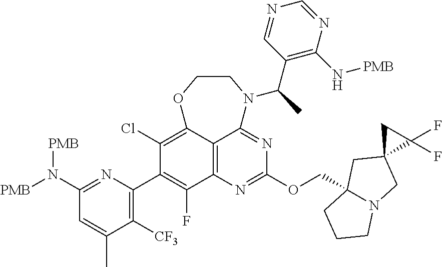

A solution of ((1R,7a′S)-2,2-difluorodihydro-1′H,3′H-spiro[cyclopropane-1,2′-pyrrolizin]-7a′(5′H)-yl)methanol (226.0 mg, crude) in tetrahydrofuran (4.0 mL) was added sodium hydride (44.0 mg, 0.5 mmol, 60% purity) and stirred at 25° C. for 15 minutes. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (343.0 mg, 0.4 mmol) was added and stirred at 25° C. for 1 hour. After completion, the resulting solution was quenched with ammonium chloride aqueous solution and extracted with ethyl acetate. Then the organic layers were combined, washed with brine, dried over anhydrous sodium sulfate and concentrated under vacuum to afford 5-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine (586.0 mg, 0.5 mmol, crude) as a yellow solid. LC-MS: (ESI, m/z): 1096.4 [M+H]⁺

Step 3: 5-((R)-1-((R)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrimidin-4-amine

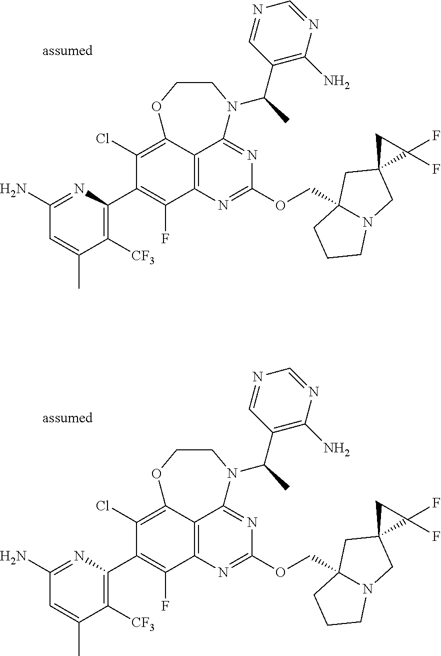

A solution of 5-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine (586.0 mg, 0.5 mmol, crude) in trifluoromethanesulfonic acid (1.0 mL) and 2,2,2-trifluoroacetic acid (10.0 mL) was stirred at 25° C. for 10 minutes. After completion, the solvent was concentrated under vacuum. The reaction mixture was diluted with ethyl acetate. The reaction mixture was adjusted to pH=8 with sodium carbonate aqueous solution, washed with sodium chloride aqueous solution and dried over anhydrous sodium sulfate and concentrated under vacuum. The residue was purified by flash chromatography on silica gel eluting with dichloromethane/methanol (11:1) to afford the product. The product was further purified by Prep-HPLC with the following conditions: Column: Xselect CSH C18 OBD Column 30*150 mm 5 μm, n; Mobile Phase A: Water (10 mmol/L NH4HCO3), Mobile Phase B: ACN; Flow rate: 60 mL/min; Gradient: 11% B to 26% B in 8 min, 26% B; Wave Length: 254/220 nm; RT1(min): 8 to afford 5-((R)-1-((R)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrimidin-4-amine (20.1 mg, 0.1 mmol, 13.9% yield) and 5-((R)-1-((S)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrimidin-4-amine (23.6 mg, 0.1 mmol, 11.8% yield).

Example 161a: ¹H NMR (400 MHz, Methanol-d₆, ppm) δ 8.42 (s, 1H), 8.33 (s, 1H), 6.60 (s, 1H), 6.46 (q, J=7.0 Hz, 1H), 4.81-4.62 (m, 2H), 4.56 (dd, J=12.5, 6.3, 1.5 Hz, 1H), 4.46-4.34 (m, 1H), 3.95 (dd, J=12.6, 5.8 Hz, 1H), 3.89-3.73 (m, 2H), 3.71-3.62 (m, 1H), 3.45 (d, J=12.5 Hz, 1H), 3.25-3.11 (m, 1H), 2.55 (dd, J=14.0, 6.2 Hz, 1H), 2.48-2.38 (m, 4H), 2.31-2.19 ((m, 2H), 2.18-2.02 (m, 2H), 1.79-1.67 (m, 5H). LC-MS: (ESI, m/z): 736.3 [M+H]⁺

Example 161b: ¹H NMR (300 MHz, DMSO-d₆, ppm) δ 8.37 (s, 1H), 8.29 (s, 1H), 6.91 (s, 2H), 6.82 (s, 2H), 6.47 (s, 1H), 6.20 (d, J=6.9 Hz, 1H), 4.56 (dd, J=12.0, 6.7 Hz, 1H), 4.36 (dd, J=12.0, 6.2 Hz, 1H), 4.26-4.04 (m, 2H), 3.79 (dd, J=15.4, 6.2 Hz, 1H), 3.46 (dd, J=15.5, 6.9 Hz, 1H), 3.15-2.90 (m, 2H), 2.76-2.65 (m, 1H), 2.59-2.52 (m, 1H), 2.36 (s, 3H), 2.13-1.95 (m, 2H), 1.89 (d, J=13.5 Hz, 1H), 1.84-1.67 (m, 2H), 1.67-1.37 (m, 6H). LC-MS: (ESI, m/z): 736.3 [M+H]⁺

Example 162a & 162b & 162c & 162d: 3-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine & 3-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine

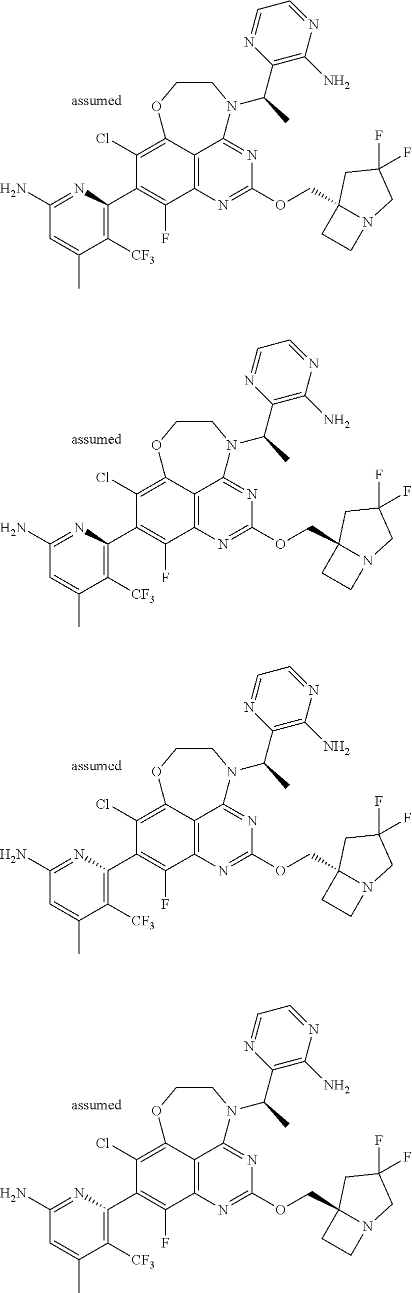

### Synthetic Route

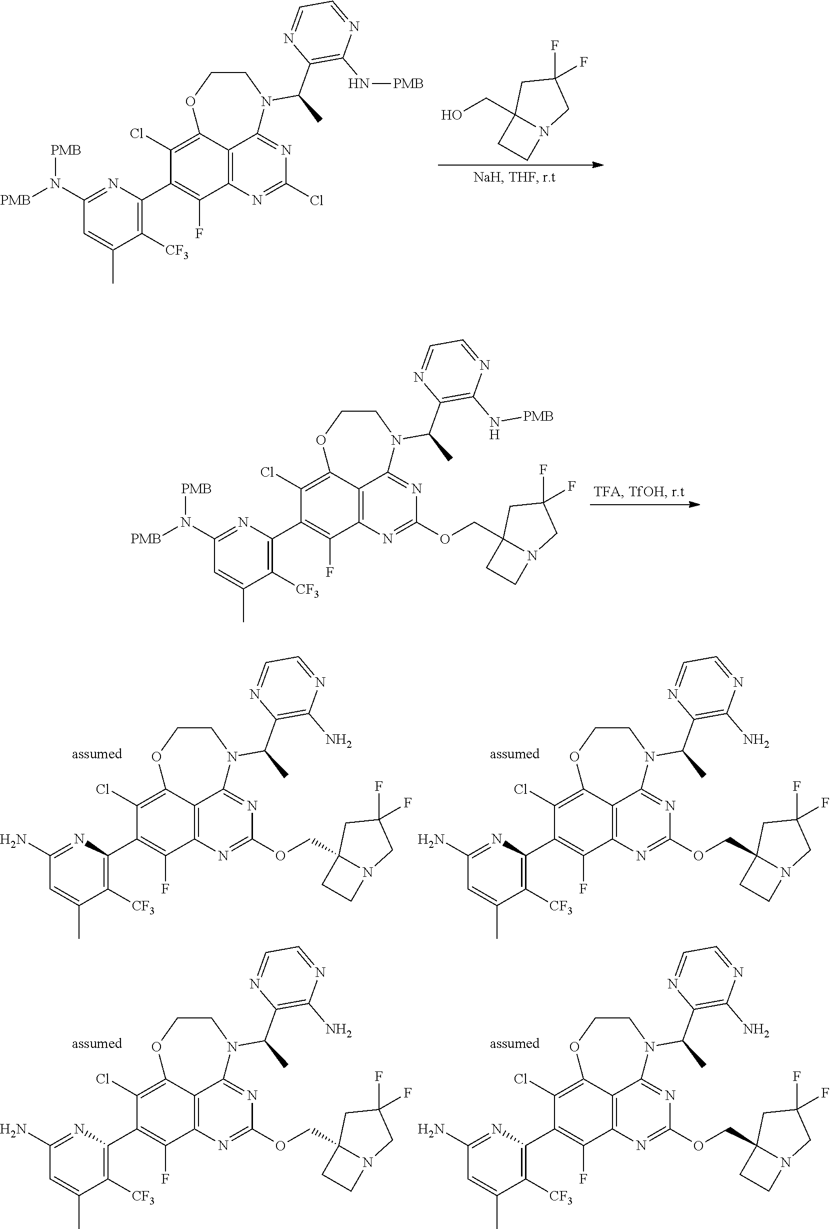

Step 1: 3-((1R)-1-(9-(6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyrazin-2-amine

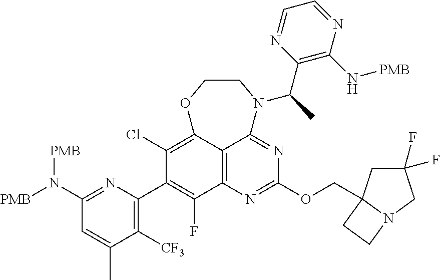

A solution of (3,3-difluoro-1-azabicyclo[3.2.0]heptan-5-yl)methanol (263.2 mg, 1.6 mmol) in tetrahydrofuran (6 mL) was added sodium hydride (64.5 mg, 2.6 mmol, 60% purity) and stirred at 25° C. for 15 minutes. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (500.0 mg, 0.5 mmol) was added and stirred at 25° C. for 1 hour. After completion, the reaction was quenched with saturated ammonium chloride solution, diluted with dichloromethane, washed with water, dried over anhydrous sodium sulfate and concentrated under vacuum to afford 3-((1R)-1-(9-(6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (900.0 mg, 0.8 mmol, crude) as a yellow solid. LC-MS: (ESI, m/z): 1056.4 [M+H]⁺

Step 2: 3-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine & 3-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine

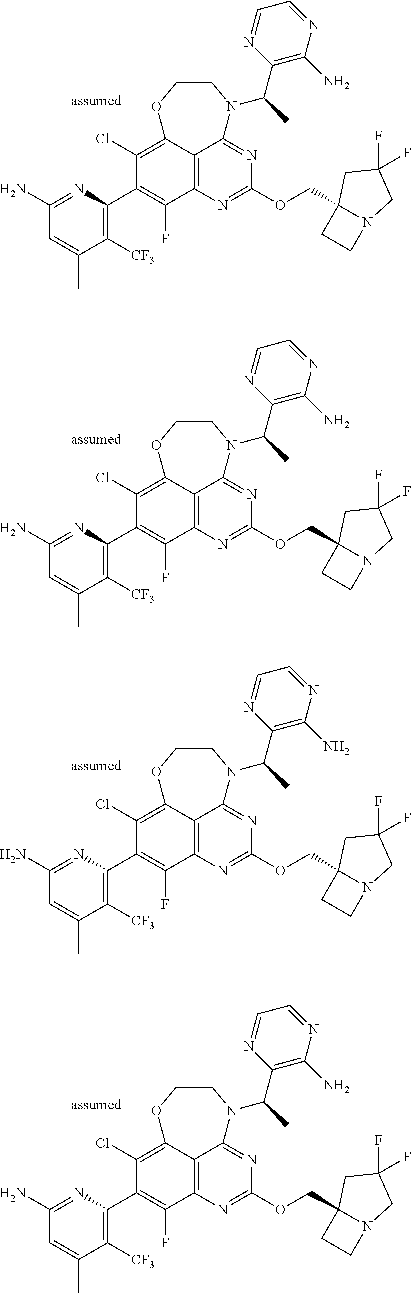

A solution of 3-((1R)-1-(9-(6-(bis(4-methoxybenzyl)amino)-4-methyl-3-(trifluoromethyl)pyridin-2-yl)-8-chloro-2-((3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (900.0 mg, 0.8 mmol, crude) in trifluoromethanesulfonic acid (1.0 mL) and 2,2,2-trifluoroacetic acid (10.0 mL) was stirred at 25° C. for 1 hour. After completion, the solvent was concentrated under vacuum. The residue was diluted with ethyl acetate and adjusted pH=8 with saturated sodium bicarbonate solution. The solution was diluted with dichloromethane, washed with water dried over anhydrous sodium sulfate and concentrated under vacuum. The residue was purified by flash chromatography on silica gel eluting with dichloromethane/methanol (11:1) to afford the product. The product was further purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 38% B to 55% B in 9 min, 55% B; Wave Length: 254/220 nm; RT1(min): 8.83 to afford faster peak (78.0 mg) and slower peak (161.5 mg) as a yellow solid. Then the faster peak (78.0 mg) was purified by Chiral-Prep-HPLC with the following conditions: Column: CHIRALPAK ID, 2*25 cm, 5 μm; Mobile Phase A: Hex:DCM=3:1(0.5% 2M NH3-MeOH)-HPLC, Mobile Phase B: IPA-HPLC: Flow rate: 20 mL/min; Gradient: 20% B to 20% B in 22 min; Wave Length: 220/254 nm; RT1(min): 14.983; RT2(min): 17.898; Sample Solvent: EtOH-HPLC; Injection Volume: 1.1 mL; Number Of Runs: 4 to afford 3-((R)-1-((R)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine (10.5 mg, 0.1 mmol, 2.7% yield) and 3-((R)-1-((R)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine (17.9 mg, 0.1 mmol, 4.3% yield). The slower peak (161.5 mg) was purified by Chiral-Prep-HPLC with the following conditions: Column: CHIRALPAK IE, 2*25 cm, 5 μm; Mobile Phase A: Hex:DCM=3:1(0.5% 2M NH3-MeOH)-HPLC, Mobile Phase B: EtOH-HPLC; Flow rate: 20 mL/min; Gradient: 20% B to 20% B in 15 min; Wave Length: 220/254 nm; RT1(min): 17.046; RT2(min): 20.554; Sample Solvent: EtOH-HPLC; Injection Volume: 0.5 mL; Number Of Runs: 9 to afford 3-((R)-1-((S)-9-(6-amino-4-methyl-3-(trifluoromethyl)pyridin-2-yl)-8-chloro-2-(((S)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine (25.8 mg, 0.1 mmol, 6.2% yield) and 3-((R)-1-((S)-9-(6-amino-4-methyl-3-(trifluoromethyl)pyridin-2-yl)-8-chloro-2-(((R)-3,3-difluoro-1-azabicyclo[3.2.0]heptan-5-yl)methoxy)-10-fluoro-5,6-dihydro-4H-[1,4]oxazepino[5,6,7-de]quinazolin-4-yl)ethyl)pyrazin-2-amine (27.0 mg, 0.1 mmol, 6.5% yield).

Example 162a: ¹H NMR (300 MHz, DMSO-d₆, ppm) δ 7.94 (d, J=2.7 Hz, 1H), 7.77 (d, J=2.7 Hz, 1H), 6.80 (s, 2H), 6.47 (s, 1H), 6.40 (s, 2H), 6.29 (q, J=6.7 Hz, 1H), 4.55 (dd, J=12.0, 6.5 Hz, 1H), 4.43-4.22 (m, 3H), 3.87 (dd, J=15.5, 6.5 Hz, 1H), 3.64 (dd, J=15.7, 6.5 Hz, 1H), 3.59-3.47 (m, 1H), 3.21-2.97 (m, 4H), 2.47-2.20 (m, 6H), 1.58 (d, J=6.8 Hz, 3H). LC-MS: (ESI, m/z): 696.2 [M+H]⁺. Chiral HPLC: Column: FCHIRALPAK ID-34.6*50 mm, 3 um, 4.6*50 mm, 3 um: Mobile Phase: (Hex:DCM=3:1)(0.1% DEA):IPA=90:10; Flow rate: 1.0 mL/min, Retention time: 2.846 min (First peak).

Example 162b: ¹H NMR (300 MHz, DMSO-d₆, ppm) δ 7.94 (d, J=2.7 Hz, 1H), 7.77 (d, J=2.7 Hz, 1H), 6.80 (s, 2H), 6.47 (s, 1H), 6.40 (s, 2H), 6.29 (q, J=6.7 Hz, 1H), 4.55 (dd, J=12.0, 6.5 Hz, 1H), 4.43-4.22 (m, 3H), 3.87 (dd, J=15.5, 6.5 Hz, 1H), 3.64 (dd, J=15.7, 6.5 Hz, 1H), 3.59-3.47 (m, 1H), 3.21-2.97 (m, 4H), 2.47-2.20 (m, 6H), 1.58 (d, J=6.8 Hz, 3H). LC-MS: (ESI, m/z): 696.2 [M+H]⁺. Chiral HPLC: Column: FCHIRALPAK ID-34.6*50 mm, 3 um, 4.6*50 mm, 3 um: Mobile Phase: (Hex:DCM=3:1)(0.1% DEA):IPA=90:10; Flowrate: 1.0 mL/min, Retention time: 4.641 min (Second peak).

Example 162c: ¹H NMR (300 MHz, DMSO-d₆, ppm) δ 7.93 (d, J=2.6 Hz, 1H), 7.76 (d, J=2.7 Hz, 1H), 6.82 (s, 2H), 6.48 (d, J=7.1 Hz, 3H), 6.26 (q, J=6.9 Hz, 1H), 4.71-4.58 (m, 1H), 4.46-4.16 (m, 3H), 4.07-3.85 (m, 1H), 3.78-3.62 (m, 1H), 3.59-3.44 (m, 1H), 3.29-2.98 (m, 3H), 2.78-2.54 (m, 2H), 2.45-2.20 (m, 5H), 1.60 (d, J=6.8 Hz, 3H). LC-MS: (ESI, m/z): 696.2 [M+H]⁺. Chiral HPLC: Column: CHIRALPAK IE-3, 4.6*50 mm, 3 um Mobile Phase: (Hex:DCM=3:1)(0.1% DEA):EtOH=80:20; Flow rate: 1.0 mL/min; Retention time: 2.613 min (First peak).

Example 162d: ¹H NMR (300 MHz, DMSO-da, ppm) δ 7.93 (d, J=2.7 Hz, 1H), 7.76 (d, J=2.7 Hz, 1H), 6.82 (s, 2H), 6.45 (d, J=13.1 Hz, 3H), 6.25 (q, J=6.7 Hz, 1H), 4.62 (dd, J=11.8, 6.8 Hz, 1H), 4.46-4.20 (m, 3H), 3.96 (dd, J=15.6, 6.2 Hz, 1H), 3.68 (dd, J=15.7, 7.0 Hz, 1H), 3.58-3.41 (m, 1H), 3.26-2.98 (m, 3H), 2.77-2.57 (m, 1H), 2.47-2.17 (m, 6H), 1.60 (d, J=6.8 Hz, 3H). LC-MS: (ESI, m/z): 696.2 [M+H]⁺. Chiral HPLC: Column: CHIRALPAK IE-3, 4.6*50 mm, 3 um Mobile Phase: (Hex:DCM=3:1)(0.1% DEA):EtOH=80:20; Flow rate: 1.0 mL/min; Retention time: 3.346 min (Second peak).

Example 163a & 163b: 3-((R)-1-((R)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrazin-2-amine

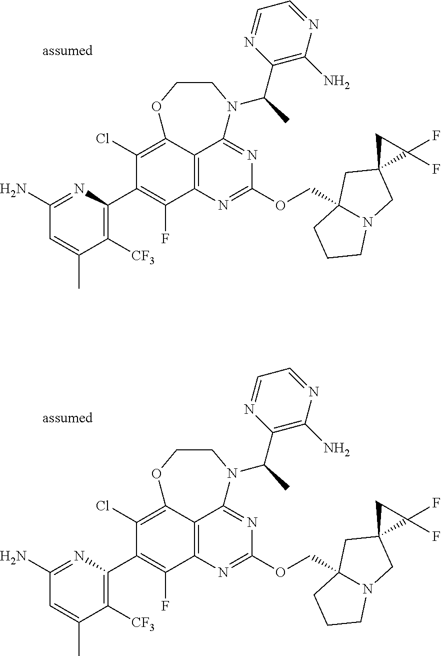

### Synthetic Route

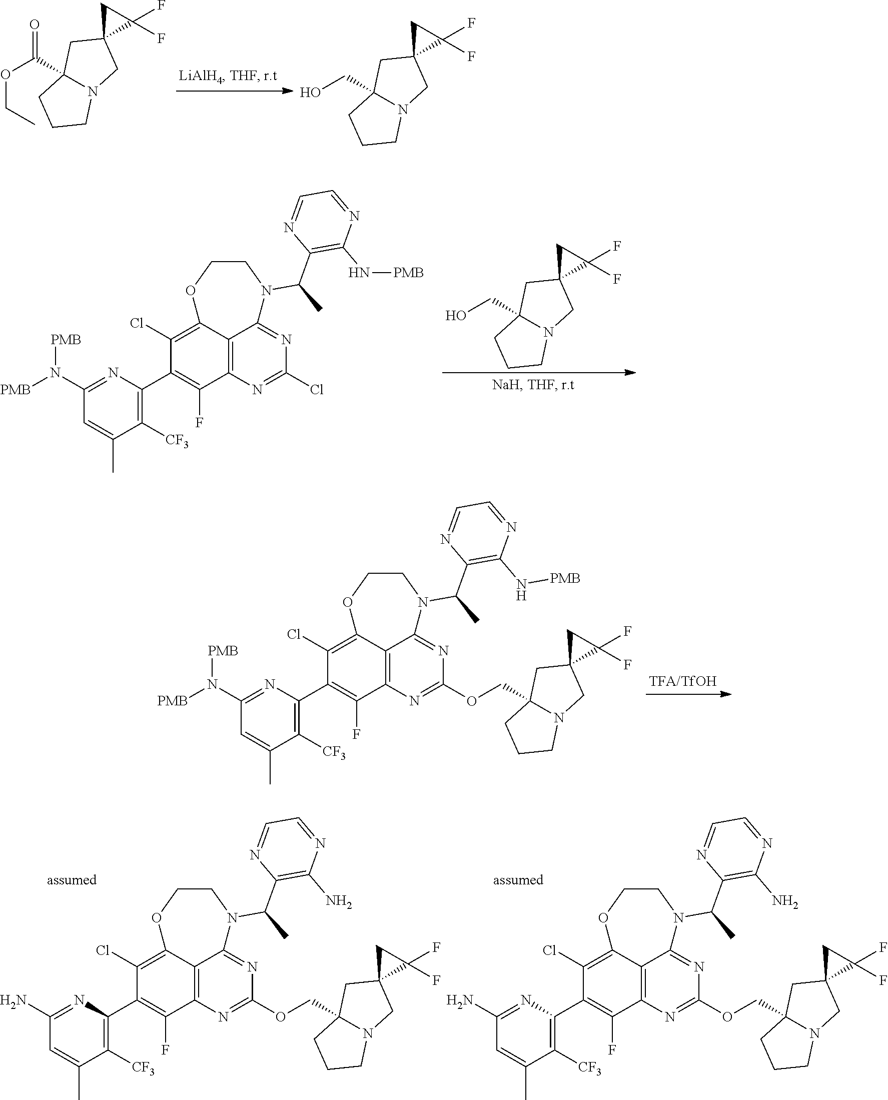

Step 1: ((1R,7a′S)-2,2-difluorodihydro-1′H,3′H-spiro[cyclopropane-1,2′-pyrrolizin]-7a′(5′H)-yl)methanol

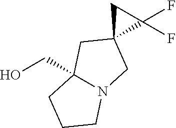 [SMILES: OC[C@@]12CCCN1C[C@]1(CC1(F)F)C2]

Under nitrogen, a solution of ethyl (1R,7a′S)-2,2-difluoro-5′-oxodihydro-1′H,3′H-spiro[cyclopropane-1,2′-pyrrolizine]-7a′(5′H)-carboxylate (400.0 mg, 1.5 mmol) in tetrahydrofuran (10.0 mL) was added lithium aluminum hydride (351.8 mg, 9.2 mmol) at 25° C. and stirred for 30 minutes at 60° C. After completion, the reaction was quenched by sodium sulfate decahydrate, diluted with tetrahydrofuran and filtered. The filter was concentrated under vacuum to afford ((1R,7a′S)-2,2-difluorodihydro-1′H,3′H-spiro[cyclopropane-1,2′-pyrrolizin]-7a′(5′H)-yl)methanol (392.7 mg, crude) as a yellow oil. LC-MS: (ESI, m/z): 204.1 [M+H]⁺

Step 2: 3-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine

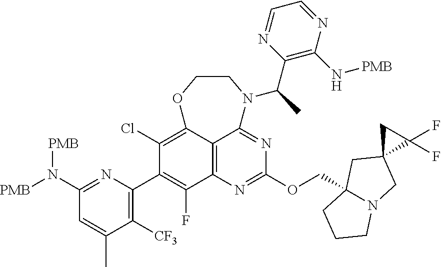

A solution of ((1R,7a′S)-2,2-difluorodihydro-1′H,3′H-spiro[cyclopropane-1,2′-pyrrolizin]-7a′(5′H)-yl)methanol (262.3 mg, 1.2 mmol) in tetrahydrofuran (4.0 mL) was added sodium hydride (51.6 mg, 2.1 mmol, 60% purity) was stirred at 25° C. for 15 minutes. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (400.0 mg, 0.40 mmol) was added and stirred at 25° C. for 1 hour. After completion, the resulting solution was quenched with ammonium chloride aqueous solution and extracted with ethyl acetate. Then the organic layers were combined, washed with brine, dried over anhydrous sodium sulfate and concentrated under vacuum. The residue was purified by flash chromatography on silica gel eluting with dichloromethane/ethyl acetate (1:1) to afford 3-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (434.0 mg, 0.3 mmol, 91.9% yield) as a yellow solid. LC-MS: (ESI, m/z): 1096.4 [M+H]⁺

Step 3: 3-((R)-1-((R)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrazin-2-amine

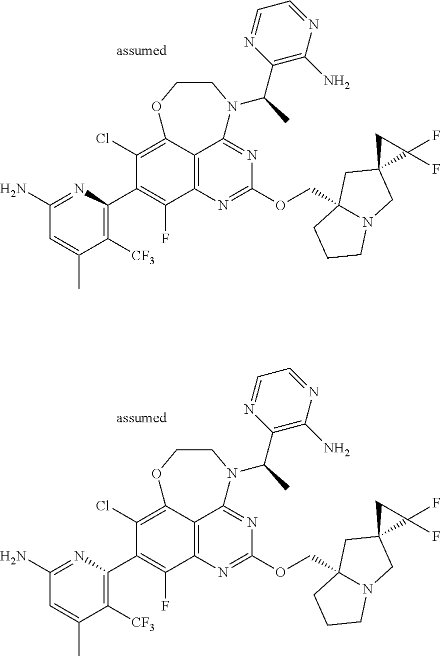

A solution of 3-((1R)-1-(9-(6-(bis(4-methoxybenzyl)amino)-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (200.0 mg, 0.2 mmol) in trifluoromethanesulfonic acid (1.0 mL) and 2,2,2-trifluoroacetic acid (10.0 mL) was stirred at 25° C. for 10 minutes. After completion, the solvent was concentrated under vacuum. The reaction mixture was diluted with ethyl acetate. The reaction mixture was adjusted to pH=8 with saturated sodium carbonate aqueous solution, washed with water, dried over anhydrous sodium sulfate and concentrated under vacuum. The residue was purified by flash chromatography on silica gel eluting with dichloromethane/methanol (11:1) to afford the product. The product was further purified by Prep-HPLC with the following conditions: Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 47% B to 72% B in 9 min, 72% B; Wave Length: 254/220 nm; RT1(min): 8.28 to afford 3-((R)-1-((R)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrazin-2-amine (42.3 mg, 0.1 mmol, 13.9% yield) & 3-((R)-1-((S)-9-(6-amino-4-methyl-3-(trifluoromethyl)pyridin-2-yl)-8-chloro-2-(((1R,7a′S)-2,2-difluorodihydro-1′H,3′H-spiro[cyclopropane-1,2′-pyrrolizin]-7a′(5′H)-yl)methoxy)-10-fluoro-5,6-dihydro-4H-[1,4]oxazepino[5,6,7-de]quinazolin-4-yl)ethyl)pyrazin-2-amine (35.8 mg, 0.1 mmol, 11.8% yield).

Example 163a: ¹H NMR (300 MHz, DMSO-d₆, ppm) δ 7.94 (d, J=2.7 Hz, 1H), 7.78 (d, J=2.7 Hz, 1H), 6.80 (s, 2H), 6.47 (s, 1H), 6.39 (s, 2H), 6.30 (q, J=6.8 Hz, 1H), 4.53 (dd, J=12.0, 6.6 Hz, 1H), 4.35 (dd, J=12.0, 6.3 Hz, 1H), 4.25-4.07 (m, 2H), 3.85 (dd, J=15.6, 6.5 Hz, 1H), 3.60 (dd, J=15.4, 6.6 Hz, 1H), 3.15-2.91 (m, 2H), 2.70 (d, J=12.0 Hz, 1H), 2.61-2.52 (m, 1H), 2.38 (s, 3H), 2.12-1.92 (m, 2H), 1.91-1.67 (m, 3H), 1.66-1.36 (in, 6H). LC-MS: (ESI, m/z): 736.3 [M+H]⁺

Example 163b: ¹H NMR (300 MHz, DMSO-d₆, ppm) δ 7.93 (d, J=2.6 Hz, 1H), 7.76 (d, J=2.7 Hz, 1H), 6.81 (s, 2H), 6.48 (d, J=6.0 Hz, 3H), 6.28 (q, J=6.7 Hz, 1H), 4.61 (dd, J=11.8, 6.9 Hz, 1H), 4.47-4.31 (in, 1H), 4.21 (d, J=10.5 Hz, 1H), 4.08 (d, J=10.5 Hz, 1H), 3.94 (dd, J=15.4, 6.2 Hz, 1H), 3.64 (dd, J=15.5, 7.0 Hz, 1H), 3.16-2.92 (m, 2H), 2.70 (d, J=11.9 Hz, 1H), 2.60-2.52 (in, 1H), 2.41-2.30 (m, 3H), 2.11-1.93 (m, 2H), 1.93-1.67 (m, 3H), 1.67-1.37 (in, 6H). LC-MS: (ESI, m/z): 736.3 [M+H]⁺

Example 164a & 164b: 5-((R)-1-((R)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrimidin-4-amine

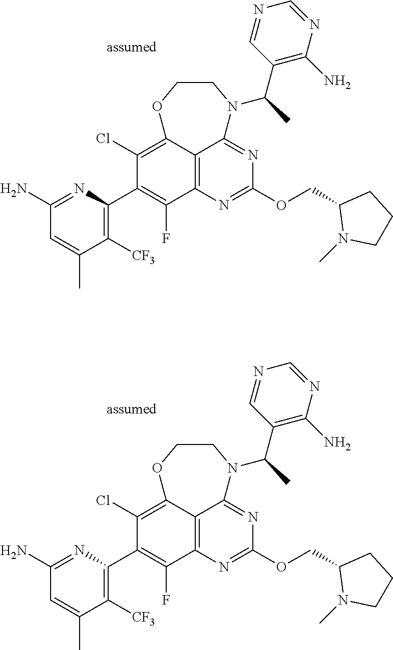

### Synthetic Route

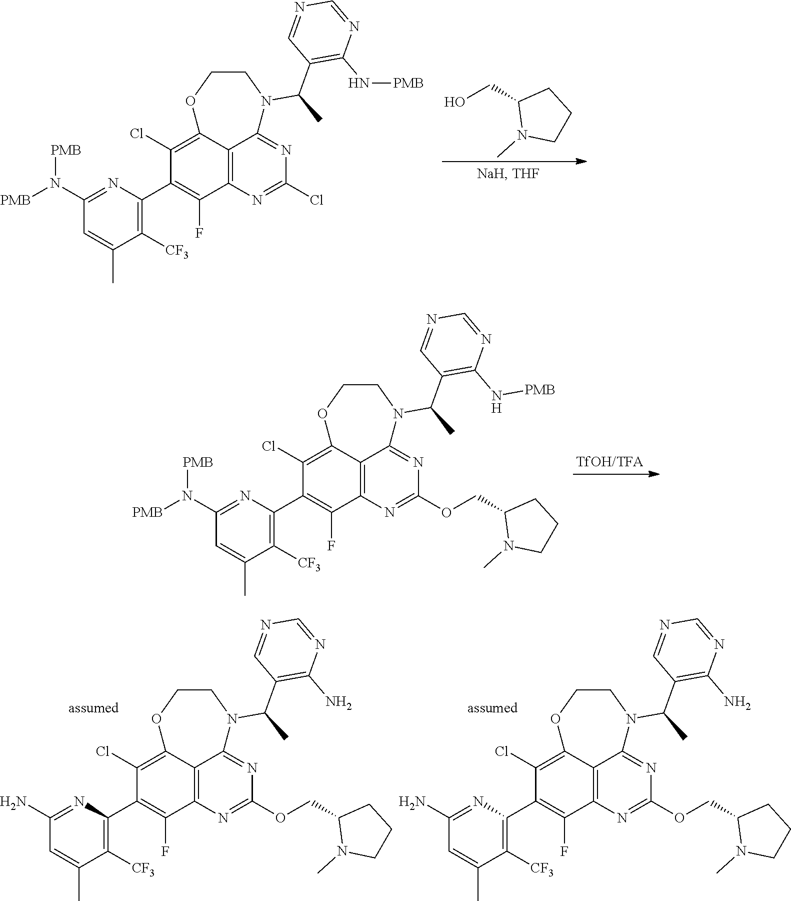

Step 1: 5-((1R)-1-(9-(6-(bis(4-methoxybenzyl)amino)-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)-N-(4-methoxybenzyl)pyrimidin-4-amine

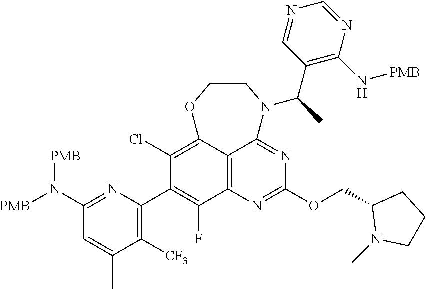

A solution of (S)-(1-methylpyrrolidin-2-yl)methanol (0.1 mL, 0.94 mmol) in tetrahydrofuran (5.0 mL) was added sodium hydride (53.8 mg, 1.34 mmol, 60% purity) and stirred at 25° C. for 15 minutes. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (250.0 mg, 0.27 mmol) was added and stirred at 25° C. for 2 hours. After completion, the reaction solution was quenched with saturated ammonium chloride solution, and dilute with ethyl acetate, washed with water, dried over anhydrous sodium sulfate and concentrated under vacuum to afford 5-((1R)-1-(9-(6-(bis(4-methoxybenzyl)amino)-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)-N-(4-methoxybenzyl)pyrimidin-4-amine (419.0 mg, 0.28 mmol) as a yellow solid. LC-MS: (ESI, m/z): 1008.4 [M+H]⁺

Step 2: 5-((R)-1-((R)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrimidin-4-amine

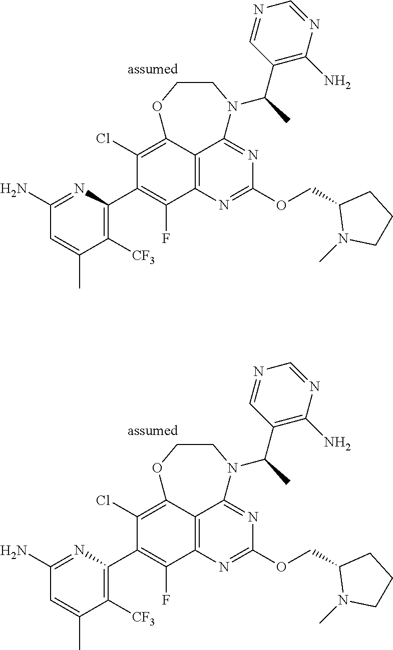

A solution of 5-((1R)-1-(9-(6-(bis(4-methoxybenzyl)amino)-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)-N-(4-methoxybenzyl)pyrimidin-4-amine (419.0 mg, 0.42 mmol) in trifluoroacetic acid (20.0 mL) and trifluoromethanesulfonic acid (1.0 mL) was stirred at 25° C. for 24 hours. After completion, the reaction solution was concentrated under vacuum and diluted with ethyl acetate. The solution was adjusted pH=8 with saturated sodium bicarbonate solution, washed with water, died over anhydrous sodium sulfate and concentrated under vacuum. The residue was purified by flash chromatography on silica gel eluting with dichloromethane/methanol (4:1). The crude product was further purified by Prep-HPLC with the following conditions. Column: Xselect CSH C18 OBD Column 30*150 mm 5 μm, n; Mobile Phase A: Water (10 mmol/L NH4HCO3), Mobile Phase B: ACN: Flow rate: 60 mL/min; Gradient: 33% B to 59% B in 9 min, 59% B; Wave Length: 254/220 nm; RT1(min): 8.18 to afford 5-((R)-1-((R)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrimidin-4-amine (13.9 mg, 0.02 mmol, 5.1% yield) and 5-((R)-1-((S)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrimidin-4-amine (14.5 mg, 0.02 mmol, 5.4% yield).

Example 164a: ¹H NMR (300 MHz, DMSO-d6) δ8.39 (s, 1H), 8.30 (s, 1H), 6.81 (s, 2H), 6.74 (s, 2H), 6.48 (d, J=1.4 Hz, 1H), 6.24-6.06 (m, 1H), 4.53-4.42 (m, 1H), 4.42-4.25 (m, 2H), 4.24-4.11 (m, 1H), 3.77-3.63 (m, 1H), 3.57-3.41 (m, 1H), 3.02-2.88 (m, 1H), 2.66-2.55 (m, 1H), 2.37 (d, J=2.8 Hz, 6H), 2.24-2.11 (m, 1H), 2.02-1.84 (m, 1H), 1.76-1.64 (m, 3H), 1.61 (d, J=6.9 Hz, 3H). LC-MS: (ESI, m/z): 648.3 [M+H]⁺

Example 164b: ¹H NMR (300 MHz, DMSO-d₆, ppm) δ 8.37 (s, 1H), 8.29 (s, 1H), 6.80 (s, 4H), 6.51-6.44 (m, 1H), 6.20 (q, J=6.9 Hz, 1H), 4.55 (dd, J=12.1, 6.6 Hz, 1H), 4.44-4.28 (m, 2H), 4.20 (dd, J=10.7, 6.4 Hz, 1H), 3.77 (dd, J=15.5, 6.4 Hz, 1H), 3.45 (dd, J=15.5, 6.8 Hz, 1H), 3.02-2.89 (m, 1H), 2.69-2.55 (m, 1H), 2.36 (d, J=2.7 Hz, 6H), 2.18 (q, J=8.5 Hz, 1H), 2.02-1.85 (m, 1H), 1.79-1.51 (m, 6H). LC-MS: (ESI, m/z): 648.3 [M+H]⁺

Example 165a & 165b: 3-((R)-1-((R)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine

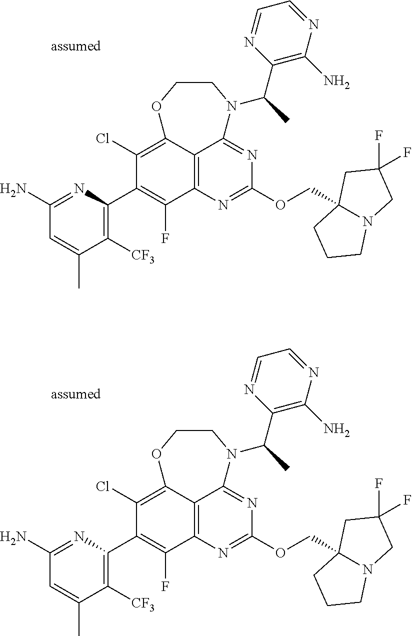

### Synthetic Route

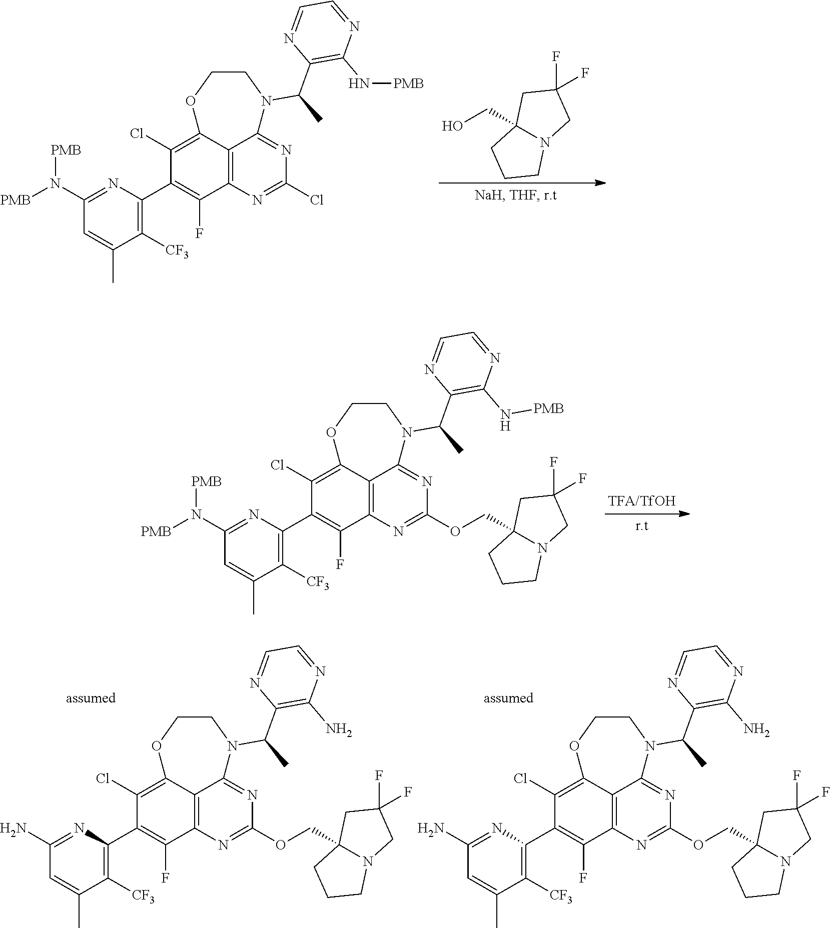

Step 1: 3-((1R)-1-(9-(6-(bis(4-methoxybenzyl)amino)-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine

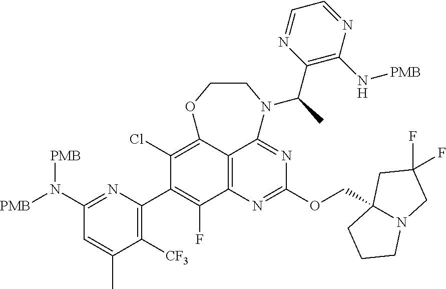

A solution of (S)-(2,2-difluorotetrahydro-1H-pyrrolizin-7a(5H)-yl)methanol (173.4 mg, 1.51 mmol) in tetrahydrofuran (5.0 mL) was added sodium hydride (86.0 mg, 2.15 mmol) and stirred at 25° C. for 15 minutes. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (400.0 mg, 0.43 mmol) was added and stirred at 25° C. for 2 hours. After completion, the reaction solution was quenched with saturated ammonium chloride solution, and dilute with ethyl acetate, washed with water, dried over anhydrous sodium sulfate and concentrated under vacuum to afford 3-((1R)-1-(9-(6-(bis(4-methoxybenzyl)amino)-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (600 mg, crude) as a red solid. LC-MS: (ESI, m/z): 1070.4 [M+H]⁺

Step 2: 3-((R)-1-((R)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine

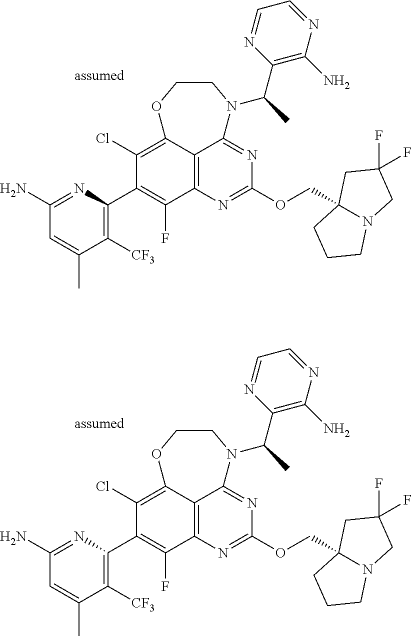

A solution of 3-((1R)-1-(9-(6-(bis(4-methoxybenzyl)amino)-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (600 mg, crude) in trifluoromethanesulfonic acid (0.5 mL) and trifluoroacetic acid (5 mL) was stirred at 25° C. for 1 hour. After completion, the reaction solution was concentrated under vacuum and diluted with ethyl acetate. The solution was adjusted pH=8 with saturated sodium bicarbonate solution, washed with water, dried over anhydrous sodium sulfate and concentrated under vacuum. The residue was purified by flash chromatography on silica gel eluting with dichloromethane/methanol (9:1)) to afford the product. The crude product was further purified by Prep-HPLC with the following conditions. Column: Xselect CSH C18 OBD Column 30*150 mm 5 μm, n: Mobile Phase A: Water (10 mmol/L NH4HCO3), Mobile Phase B: ACN; Flow rate: 60 mL/min; Gradient: 33% B to 59% B in 9 min, 59% B; Wave Length: 254/220 nm; RT1(min): 8.18 to afford 3-((R)-1-((R)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine (34.8 mg, 0.05 mmol, 14.3% yield) and 3-((R)-1-((S)-9-(6-amino-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine (49.1 mg, 0.07 mmol, 20.1% yield).

Example 165a: ¹H NMR (300 MHz, DMSO-d₆, ppm) δ 7.95 (d, J=2.7 Hz, 1H), 7.78 (d, J=2.6 Hz, 1H), 6.81 (s, 2H), 6.51-6.45 (m, 1H), 6.39 (s, 2H), 6.28 (q, J=6.7 Hz, 1H), 4.53 (dd, J=11.9, 6.5 Hz, 1H), 4.35 (dd, J=11.8, 6.3 Hz, 1H), 4.19-3.99 (m, 2H), 3.85 (dd, J=15.6, 6.5 Hz, 1H), 3.59 (dd, J=15.5, 6.6 Hz, 1H), 3.42-3.23 (m, 1H), 3.15-2.96 (m, 2H), 2.77-2.62 (m, 1H), 2.48-2.40 (m, 1H), 2.40-2.21 (m, 4H), 2.07-1.95 (m, 1H), 1.92-1.67 (m, 3H), 1.58 (d, J=6.7 Hz, 3H).

Example 165b: ¹H NMR (300 MHz, DMSO-d₆, ppm) δ 7.94 (d, J=2.6 Hz, 1H), 7.77 (d, J=2.7 Hz, 1H), 6.81 (s, 2H), 6.48 (d, J=4.6 Hz, 3H), 6.27 (q, J=6.8 Hz, 1H), 4.61 (dd, J=11.8, 6.9 Hz, 1H), 4.46-4.29 (m, 1H), 4.19-3.84 (m, 3H), 3.63 (dd, J=15.6, 7.0 Hz, 1H), 3.16-2.97 (m, 2H), 2.78-2.63 (m, 1H), 2.44-2.16 (m, 5H), 2.11-1.94 (m, 1H), 1.92-1.66 (m, 4H), 1.59 (d, J=6.8 Hz, 3H). LC-MS: (ESI, m/z): 710.4 [M+H]⁺

Example 166a & 166b: 3-((R)-1-((R)-9-(6-amino-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine

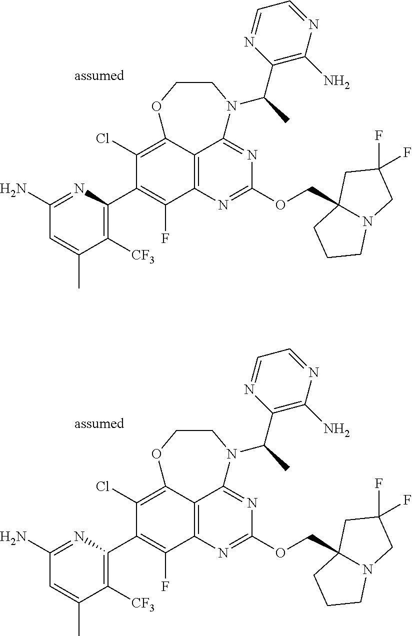

### Synthetic Route

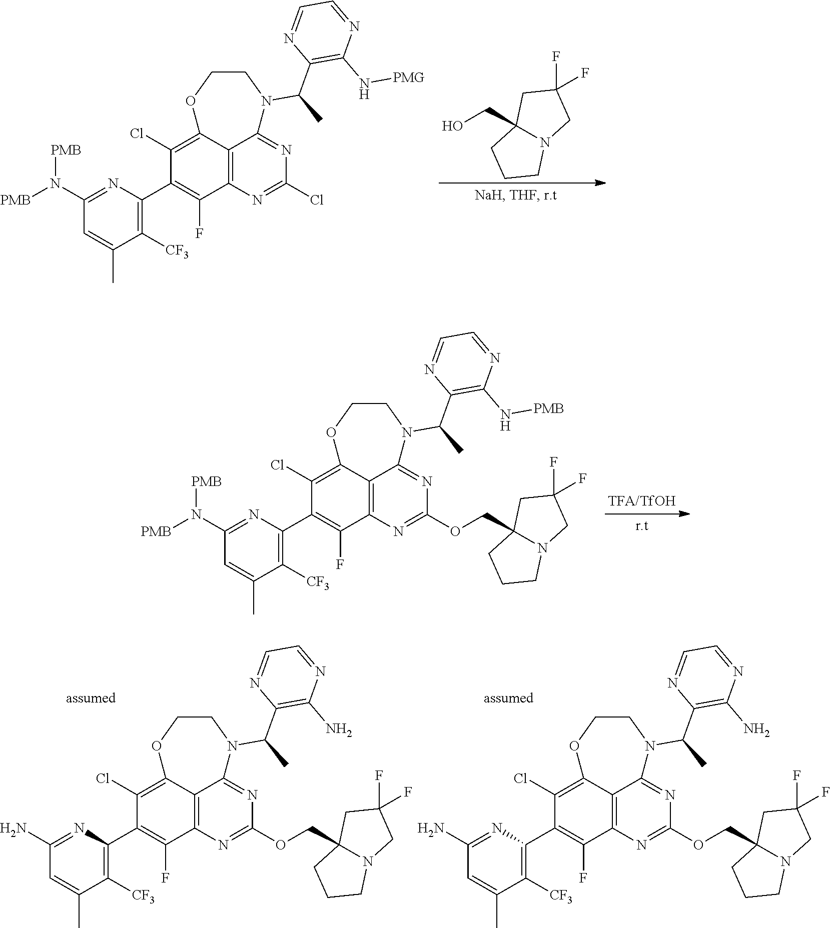

Step 1: 3-((1R)-1-(9-(6-(bis(4-methoxybenzyl)amino)-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine

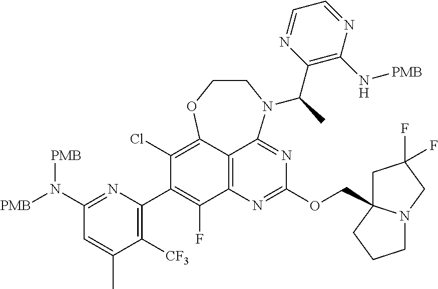

A solution of (R)-(2,2-difluorotetrahydro-1H-pyrrolizin-7a(5H)-yl)methanol (304.9 mg, 1.72 mmol) was added sodium hydride (86.0 mg, 2.15 mmol, 60% purity) in tetrahydrofuran (6.0 mL) and stirred at 25° C. for 15 minutes. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (400.0 mg, 0.43 mmol) was added and stirred at 25° C. for 1 hour. After completion, the reaction solution was quenched with saturated ammonium chloride solution, and dilute with ethyl acetate, washed with water, dried over anhydrous sodium sulfate and concentrated under vacuum to afford 3-((1R)-1-(9-(6-(bis(4-methoxybenzyl)amino)-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (600 mg, crude) as a red solid. LC-MS: (ESI, m/z): 1070.4 [M+H]⁺

Step 2: 3-((R)-1-((R)-9-(6-amino-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine

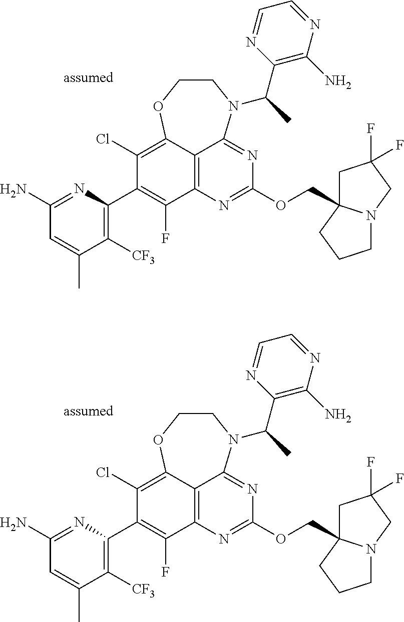

A solution of 3-((1R)-1-(9-(6-(bis(4-methoxybenzyl)amino)-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (600 mg, crude) in trifluoromethanesulfonic acid (1 mL) and trifluoroacetic acid (10 mL) was stirred at 25° C. for 1 hour. After completion, the reaction solution was concentrated under vacuum and diluted with ethyl acetate. The solution was adjusted pH=8 with saturated sodium bicarbonate solution, washed with water, dried over anhydrous sodium sulfate and concentrated under vacuum. The residue was purified by flash chromatography on silica gel eluting with dichloromethane/methanol (1:9) to afford the product. The crude product was further purified by Prep-HPLC with the following conditions. Column: Xselect CSH C18 OBD Column 30*150 mm 5 μm, n: Mobile Phase A: Water (10 mmol/L NH4HCO3), Mobile Phase B: ACN; Flow rate: 60 mL/min; Gradient: 33% B to 59% B in 9 min, 59% B; Wave Length: 254/220 nm; RT1(min): 8.18 to afford 3-((R)-1-((R)-9-(6-amino-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine (87.7 mg, 0.12 mmol, 37.2% yield) and 3-((R)-1-((S)-9-(6-amino-4-methyl-3-(trifluoromethyl)pyridin-2-yl)-8-chloro-2-(((R)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrazin-2-amine (11.2 mg, 0.02 mmol, 4.7% yield).

Example 166a: ¹H NMR (300 MHz, DMSO-d₆, ppm) δ 7.94 (d, J=2.7 Hz, 1H), 7.78 (d, J=2.7 Hz, 1H), 6.80 (s, 2H), 6.47 (s, 1H), 6.35 (s, 2H), 6.28 (q, J=6.7 Hz, 1H), 4.53 (dd, J=12.0, 6.5 Hz, 1H), 4.35 (dd, J=11.9, 6.4 Hz, 1H), 4.09 (s, 2H), 3.85 (dd, J=15.7, 6.4 Hz, 1H), 3.60 (dd, J=15.6, 6.6 Hz, 1H), 3.43-3.34 (m, 1H), 3.16-2.95 (m, 2H), 2.78-2.64 (m, 1H), 2.44-2.22 (m, 5H), 2.05-1.95 (m, 1H), 1.92-1.67 (m, 3H), 1.58 (d, J=6.8 Hz, 3H). LC-MS: (ESI, m/): 710.4 [M+H]⁺

Example 166b: ¹H NMR (300 MHz, DMSO-d₆, ppm) δ 7.93 (d, J=2.6 Hz, 1H), 7.77 (d, J=2.6 Hz, 1H), 6.81 (s, 2H), 6.46 (d, J=8.7 Hz, 3H), 6.26 (q, J=6.7 Hz, 1H), 4.69-4.52 (m, 1H), 4.38 (dd, J=11.8, 6.1 Hz, 1H), 4.19-4.00 (m, 2H), 3.94 (dd, J=16.0, 5.7 Hz, 1H), 3.64 (dd, J=15.8, 6.8 Hz, 1H), 3.46-3.31 (m, 1H), 3.18-2.95 (m, 2H), 2.70 (q, J=8.2 Hz, 1H), 2.46-2.22 (m, 5H), 2.06-1.94 (m, 1H), 1.93-1.67 (m, 3H), 1.59 (d, J=6.8 Hz, 3H). LC-MS: (ESI, m/z): 710.4 [M+H]⁺

Example 167a & 167b: 3-((R)-1-((R)-9-(6-amino-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)pyrazin-2-amine

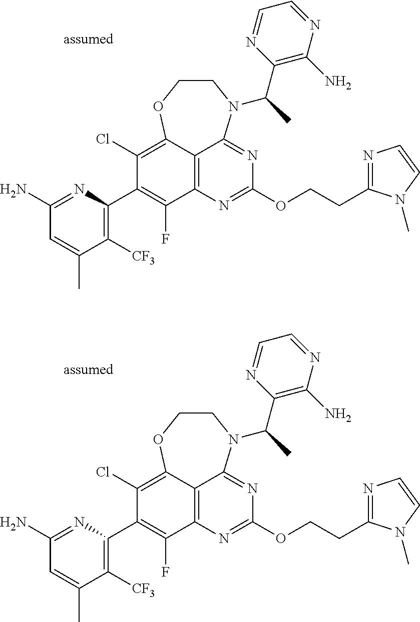

### Synthetic Route

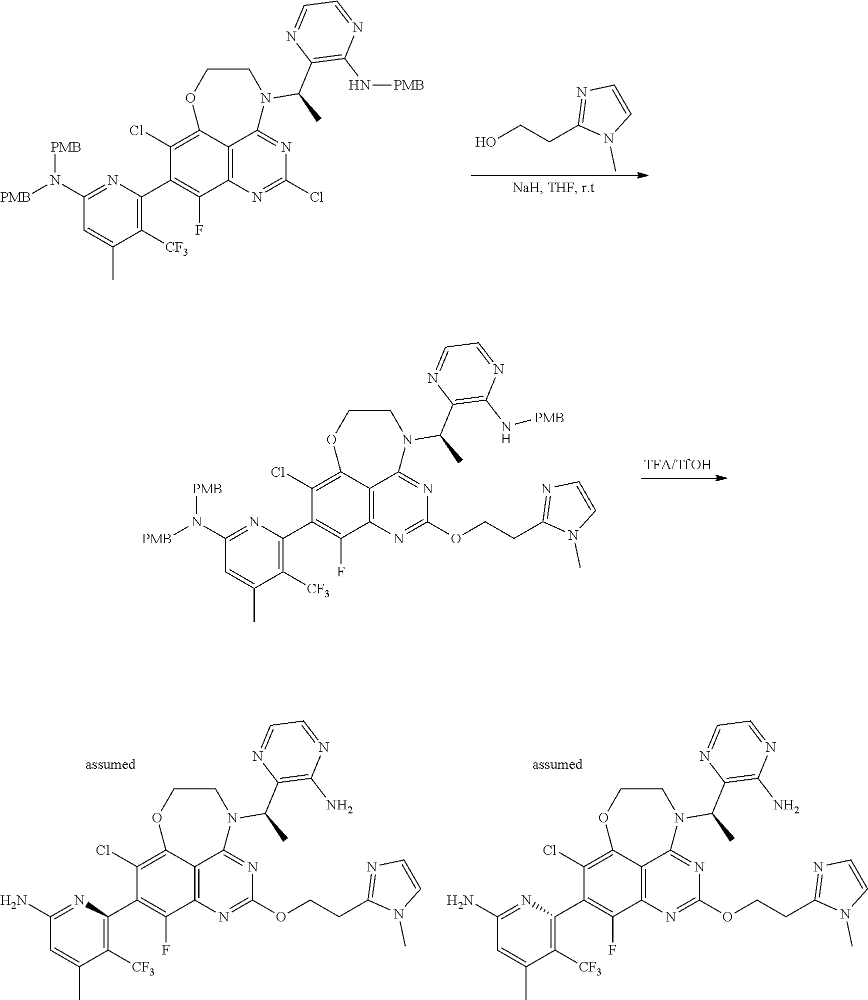

Step 1: 3-((1R)-1-(9-(6-(bis(4-methoxybenzyl)amino)-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)-N-(4-methoxybenzyl)pyrazin-2-amine

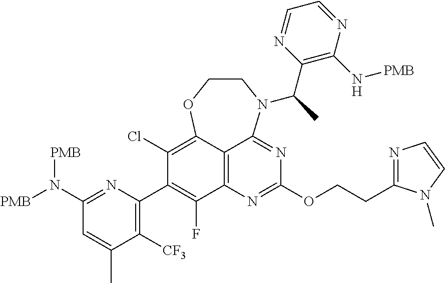

A solution of 2-(1-methyl-1H-imidazol-2-yl)ethan-1-ol (101.7 mg, 0.8 mmol) in tetrahydrofuran (3.0 mL) was added sodium hydride (38.7 mg, 1.6 mmol, 60% purity) and was stirred at 25° C. for 15 minutes. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (250.0 mg, 0.2 mmol) was added and stirred at 25° C. for 1 hour. After completion, the resulting solution was quenched with saturated ammonium chloride solution and extracted with ethyl acetate. Then the organic layers were combined, washed with brine, dried over anhydrous sodium sulfate and concentrated under vacuum. The residue was purified by flash chromatography on silica gel eluting with dichloromethane/ethyl acetate (1:1) to afford 3-((1R)-1-(9-(6-(bis(4-methoxybenzyl)amino)-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)-N-(4-methoxybenzyl)pyrazin-2-amine (282.5 mg, crude) as a yellow solid. LC-MS: (ESI, m/z): 1019.4 [M+H]⁺

Step 2: 3-((R)-1-((R)-9-(6-amino-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)pyrazin-2-amine&3-((R)-1-((S)-9-(6-amino-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)pyrazin-2-amine

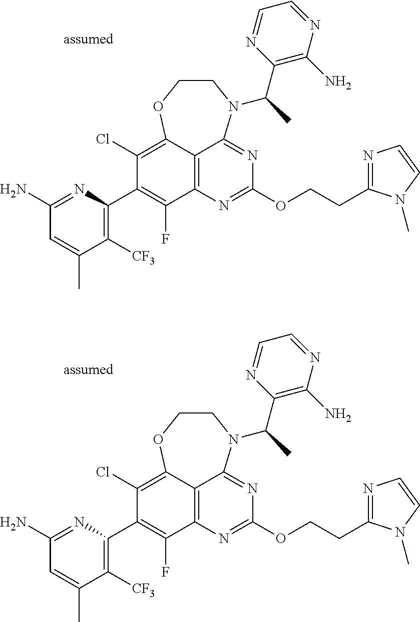

A solution of 3-((1R)-1-(9-(6-(bis(4-methoxybenzyl)amino)-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)-N-(4-methoxybenzyl)pyrazin-2-amine (200.0 mg, 0.2 mmol) in trifluoromethanesulfonic acid (1.0 mL) and 2,2,2-trifluoroacetic acid (10.0 mL) was stirred at 25° C. for 10 minutes. After completion, the solvent was concentrated under vacuum. The reaction mixture was diluted with ethyl acetate and adjusted to pH=8 with aqueous sodium carbonate solution. The organic layer was washed with sodium chloride aqueous solution. The organic layer was dried over anhydrous sodium sulfate and concentrated under vacuum. The residue was purified by flash chromatography on silica gel eluting with dichloromethane/methanol (11:1) to afford the product. The product was further purified by Prep-HPLC with the following conditions: Column, XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 38% B to 45% B in 6.5 min; Wave Length: 220 nm; RT1(min): 6.6 to afford 3-((R)-1-((R)-9-(6-amino-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)pyrazin-2-amine (15.7 mg, 0.1 mmol, 11.7% yield) and 3-((R)-1-((S)-9-(6-amino-4-methyl-3-(trifluoromethyl)pyridin-2-yl)-8-chloro-10-fluoro-2-(2-(1-methyl-1H-imidazol-2-yl)ethoxy)-5,6-dihydro-4H-[1,4]oxazepino[5,6,7-de]quinazolin-4-yl)ethyl)pyrazin-2-amine (10.5 mg, 0.1 mmol, 8.1% yield).

Example 167a: ¹H NMR (300 MHz, DMSO-d₆, ppm) δ 7.95 (d, J=2.6 Hz, 1H), 7.78 (d, J=2.6 Hz, 1H), 7.02 (d, J=1.2 Hz, 1H), 6.80 (s, 2H), 6.76 (d, J=1.2 Hz, 1H), 6.47 (s, 1H), 6.39-6.23 (m, 3H), 4.74-4.57 (m, 2H), 4.52 (dd, J=12.3, 6.5 Hz, 1H), 4.35 (dd, J=12.1, 5.9 Hz, 1H), 3.83 (dd, J=15.0, 6.7 Hz, 1H), 3.66-3.52 (m, 4H), 3.10 (t, J=7.0 Hz, 2H), 2.36 (s, 3H), 1.57 (d, J=6.8 Hz, 3H). LC-MS: (ESI, m/z): 659.2 [M+H]⁺

Example 167b: ¹H NMR (300 MHz, DMSO-d₆, ppm) δ 7.94 (d, J=2.7 Hz, 1H), 7.77 (d, J=2.7 Hz, 1H), 7.03 (d, J=1.3 Hz, 1H), 6.81 (s, 2H), 6.77 (d, J=1.2 Hz, 1H), 6.47 (s, 1H), 6.41 (s, 2H), 6.28 (q, J=6.7 Hz, 1H), 4.70-4.54 (m, 3H), 4.37 (dd, J=11.9, 6.1 Hz, 1H), 3.91 (dd, J=15.4, 6.2 Hz, 1H), 3.71-3.56 (m, 4H), 3.10 (t, J=7.0 Hz, 2H), 2.36 (s, 3H), 1.58 (d, J=6.8 Hz, 3H). LC-MS: (ESI, m/z): 659.3 [M+H]⁺

Example 168a & 168b: 6-((R)-4-((R)-1-(2-amino-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((S)-1-(2-amino-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

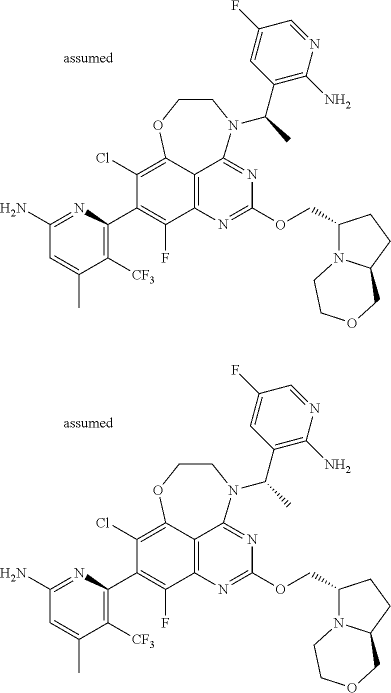

### Synthetic Route

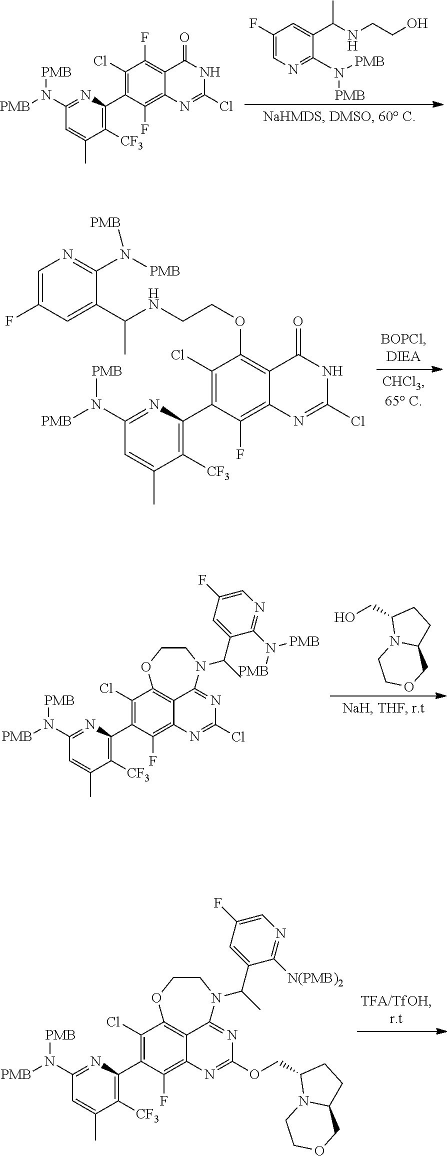

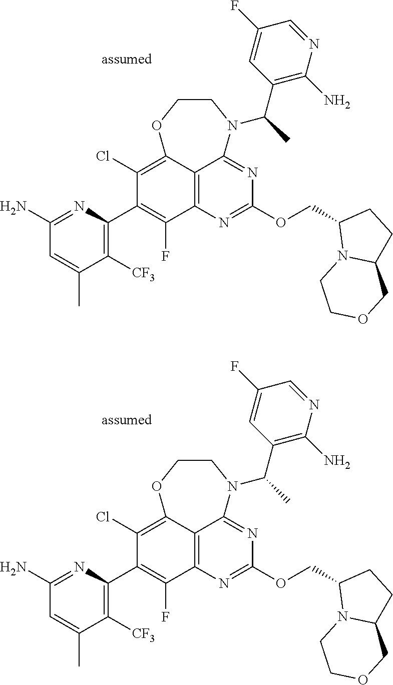

Step 1: (7R)-7-(6-(bis(4-methoxybenzyl)amino)-4-methyl-3-(trifluoromethyl)pyridin-2-yl)-5-(2-((1-(2-(bis(4-methoxybenzyl)amino)-5-fluoropyridin-3-yl)ethyl)amino)ethoxy)-2,6-dichloro-8-fluoroquinazolin-4(3H)-one

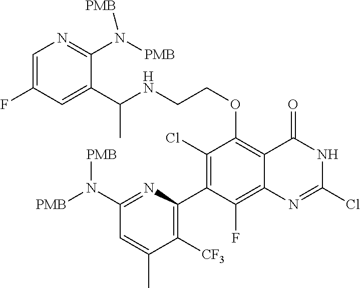

Similar to as described in General Procedure A. A solution of 2-((1-(2-(bis(4-methoxybenzyl)amino)-5-fluoropyridin-3-yl)ethyl)amino)ethan-1-ol (1.73 g, 3.94 mmol) in dimethyl sulfoxide (20 mL) was added sodium bis(trimethylsilyl)amide (1.0 M in tetrahydrofuran), (11.2 mL, 11.2 mmol) and stirred at 25° C. for 30 minutes. Then the solution was added into a solution of (R)-7-(6-(bis(4-methoxybenzyl)amino)-4-methyl-3-(trifluoromethyl)pyridin-2-yl)-2,6-dichloro-5,8-difluoroquinazolin-4(3H)-one (2.50 g, 3.76 mmol) in dimethyl sulfoxide (20 mL) and stirred at 60° C. for 1 hour. After completion, the reaction was quenched with saturated ammonium chloride solution. The resulting solution was diluted with water, extracted with ethyl acetate and washed with water. The organic layer was dried over anhydrous sodium sulfate and concentrated under vacuum to afford (7R)-7-(6-(bis(4-methoxybenzyl)amino)-4-methyl-3-(trifluoromethyl)pyridin-2-yl)-5-(2-((1-(2-(bis(4-methoxybenzyl)amino)-5-fluoropyridin-3-yl)ethyl)amino)ethoxy)-2,6-dichloro-8-fluoroquinazolin-4(3H)-one (6.00 g, crude) as a yellow solid. LC-MS: (ESI, m/z): 1084.3 [M+H]⁺

Step 2: 6-((9R)-4-(1-(2-(bis(4-methoxybenzyl)amino)-5-fluoropyridin-3-yl)ethyl)-2,8-dichloro-10-fluoro-5,6-dihydro-4H-[1, 4]oxazepino[5,6,7-de]quinazolin-9-yl)-N,N-bis(4-methoxybenzyl)-4-methyl-5-(trifluoromethyl)pyridin-2-amine

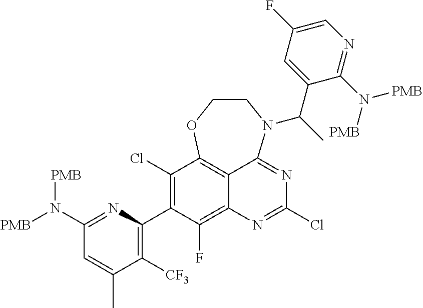

Similar to as described in General Procedure B. A solution of (7R)-7-(6-(bis(4-methoxybenzyl)amino)-4-methyl-3-(trifluoromethyl)pyridin-2-yl)-5-(2-((1-(2-(bis(4-methoxybenzyl)amino)-5-fluoropyridin-3-yl)ethyl)amino)ethoxy)-2,6-dichloro-8-fluoroquinazolin-4(3H)-one (6.00 g, crude) and N,N-diisopropylethylamine (3.8 mL, 22.1 mmol) in chloroform (60 mL). Then bis(2-oxo-3-oxazolidinyl)phosphinic chloride (2.81 g, 11.08 mmol) was added and stirred at 65° C. for 1 hour. After completion, the solvent was concentrated under vacuum. The residue was diluted with ethyl acetate. Then washed with water and dried over anhydrous sodium sulfate and concentrated under vacuum. The residue was purified by flash chromatography on silica gel eluting with ethyl acetate/dichloromethane (1:9) to afford 6-((9R)-4-(1-(2-(bis(4-methoxybenzyl)amino)-5-fluoropyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (2.30 g, 2.09 mmol, 37.7% yield) as a yellow solid. LC-MS: (ESI, m/z): 1066.3 [M+H]⁺

Step 3: 6-((9R)-4-(1-(2-(bis(4-methoxybenzyl)amino)-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

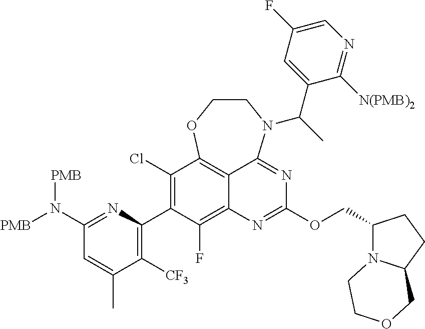

A solution of ((6S,8aS)-hexahydro-1H-pyrrolo[2,1-c][1,4]oxazin-6-yl)methanol (176.8 mg, 1.1 mmol) in tetrahydrofuran (5 mL) was added sodium hydride (74.9 mg, 1.8 mmol, 60% purity) and stirred at 25° C. for 30 minutes. Then 6-((9R)-4-(1-(2-(bis(4-methoxybenzyl)amino)-5-fluoropyridin-3-yl)ethyl)-2,8-dichloro-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (400.0 mg, 0.3 mmol) was added and stirred at 25° C. for 8 hours. After completion, the reaction was quenched with saturated ammonium chloride solution. The resulting solution was diluted with ethyl acetate, washed with water, dried over anhydrous sodium sulfate and concentrated under vacuum to afford 6-((9R)-4-(1-(2-(bis(4-methoxybenzyl)amino)-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (600 mg, crude) as a yellow solid. LC-MS: (ESI, m/z): 1087.5 [M+H]⁺

Step 4: 6-((R)-4-((R)-1-(2-amino-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine & 6-((R)-4-((S)-1-(2-amino-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine

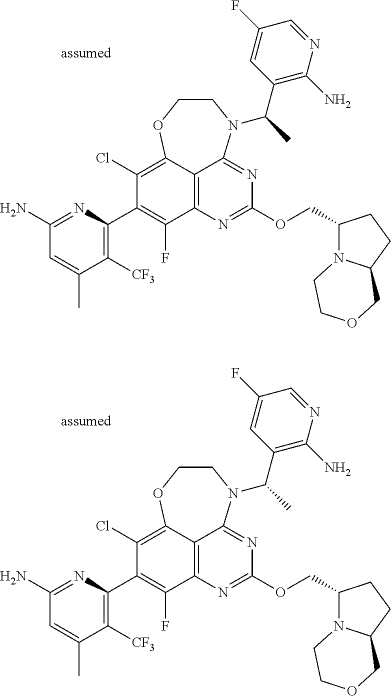

A solution of 6-((9R)-4-(1-(2-(bis(4-methoxybenzyl)amino)-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (600 mg, crude) in trifluoromethanesulfonic acid (1 mL) and 2,2,2-trifluoroacetic acid (10 mL) was stirred at 25° C. for 1 hour. After completion, the solvent was concentrated under vacuum. The residue was diluted with ethyl acetate, adjusted to PH=8 with saturated sodium bicarbonate solution. The solution was washed with brine, dried over anhydrous sodium sulfate and concentrated under vacuum. The residue was purified by flash chromatography on silica gel eluting with dichloromethane/methanol (11:1) to afford two product. The two product was further purified by Prep-HPLC with the following conditions: Column: XBridge Prep OBD C18 Column, 30*150 mm, 5 μm: Mobile Phase A: Water (10 mmol/L NH4HCO3), Mobile Phase B: ACN; Flow rate: 60 mL/min; Gradient: 17% B to 42% B in 9 min, 42% B; Wave Length: 254/220 nm; RT1(min): 8.9 to afford 6-((R)-4-((R)-1-(2-amino-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (50.1 mg, 0.07 mmol, 14% yield) and 6-((R)-4-((S)-1-(2-amino-5-fluoropyridin-3-yl)ethyl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-9-yl)-4-methyl-5-(trifluoromethyl)pyridin-2-amine (59.0 mg, 0.08 mmol, 16.5% yield).

Example 168a: ¹H NMR (300 MHz, DMSO-d₆, ppm) δ 7.96 (d, J=2.8 Hz, 1H), 7.61 (dd, J=9.6, 2.9 Hz, 1H), 6.81 (s, 2H), 6.48 (s, 1H), 6.21 (q, J=6.8 Hz, 1H), 5.68 (s, 2H), 4.51-4.26 (m, 3H), 4.16 (dd, J=10.9, 5.7 Hz, 1H), 3.78-3.35 (m, 6H), 3.14 (t, J=10.3 Hz, 1H), 3.02-2.75 (m, 3H), 2.37 (d, J=2.2 Hz, 3H), 2.15-1.96 (m, 1H), 1.84-1.67 (m, 1H), 1.67-1.50 (m, 4H), 1.39-1.18 (m, 1H). LC-MS: (ESI, m/z): 707.2 [M+H]⁺

Example 168b: ¹H NMR (300 MHz, DMSO-d₆, ppm) δ 7.95 (d, J=2.8 Hz, 1H), 7.60 (dd, J=9.6, 2.9 Hz, 1H), 6.81 (s, 2H), 6.48 (s, 1H), 6.24 (d, J=6.8 Hz, 1H), 5.77 (s, 2H), 4.58-4.26 (m, 3H), 4.11 (dd, J=10.9, 5.4 Hz, 1H), 3.73 (d, J=6.3 Hz, 1H), 3.68-3.38 (m, 5H), 3.14 (t, J=10.3 Hz, 1H), 3.04-2.78 (m, 3H), 2.37 (d, J=2.3 Hz, 3H), 2.15-1.97 (m, 1H), 1.86-1.68 (m, 1H), 1.59 (d, J=6.8 Hz, 4H), 1.37-1.20 (m, 1H). LC-MS: (ESI, m/z): 707.2 [M+H]⁺

Example 169a and 169b: 5-((R)-1-((R)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyrimidin-4-amine

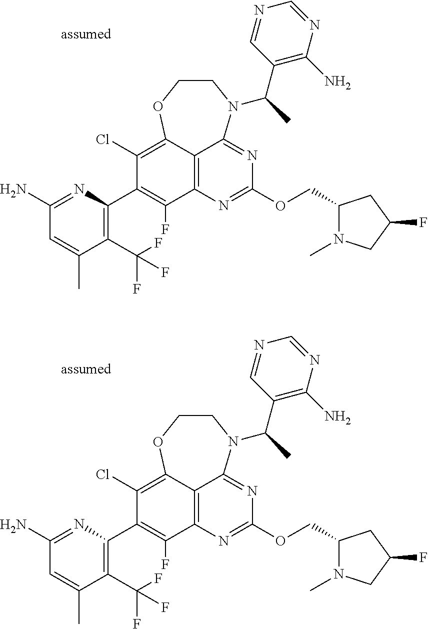

### Synthetic Route

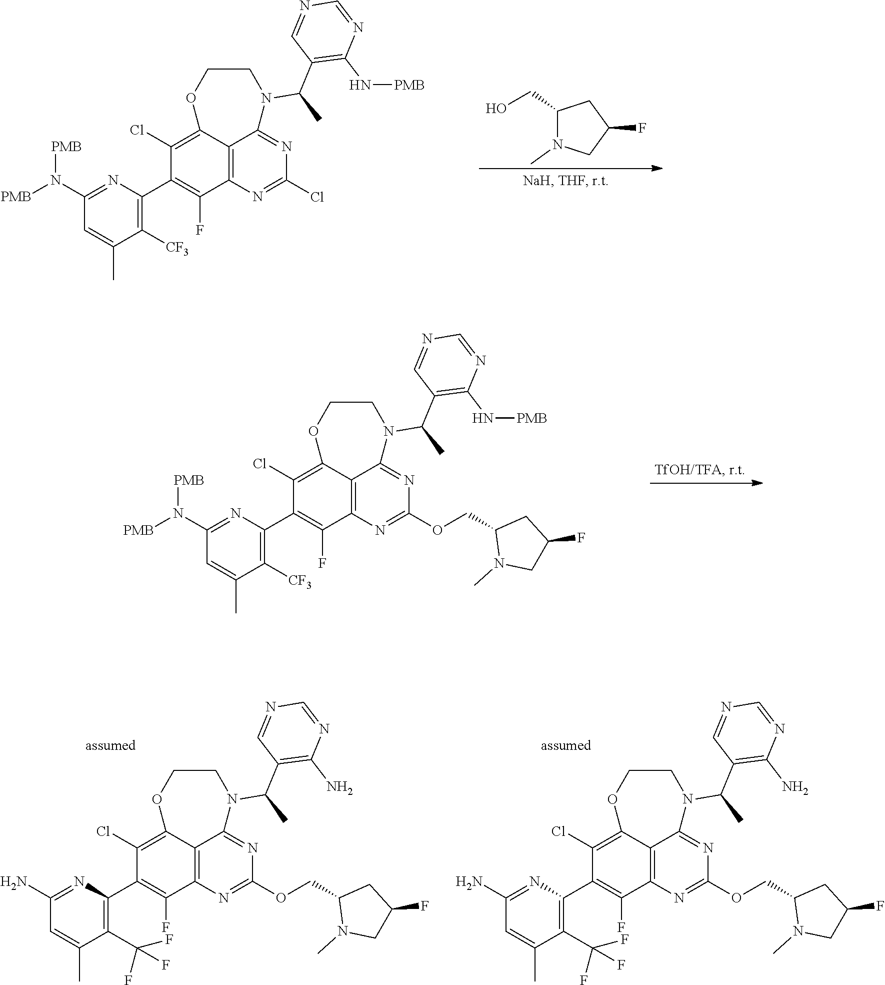

Step 1: 5-((1R)-1-(9-(6-(bis(4-methoxybenzyl)amino)-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)-N-(4-methoxybenzyl)pyrimidin-4-amine

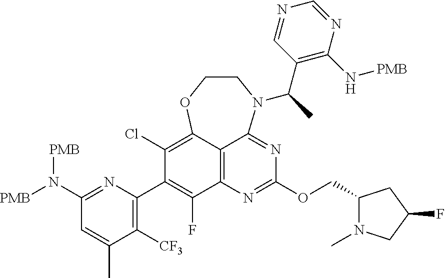

Under nitrogen, to a solution of ((2S,4R)-4-fluoro-1-methylpyrrolidin-2-yl)methanol (150.3 mg, 1.13 mmol) in tetrahydrofuran (4 mL) was added sodium hydride (75.2 mg, 1.88 mmol, 60% dispersion in mineral oil), the mixture was stirred at 0° C. for 10 minutes. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (350.0 mg, 0.38 mmol) was added and stirred at 25° C. for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The crude product would be directly used in the next step without purification. LCMS (ESI, m/z): 1026.4 [M+H]⁺.

Step 2: 5-((R)-1-((R)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyrimidin-4-amine

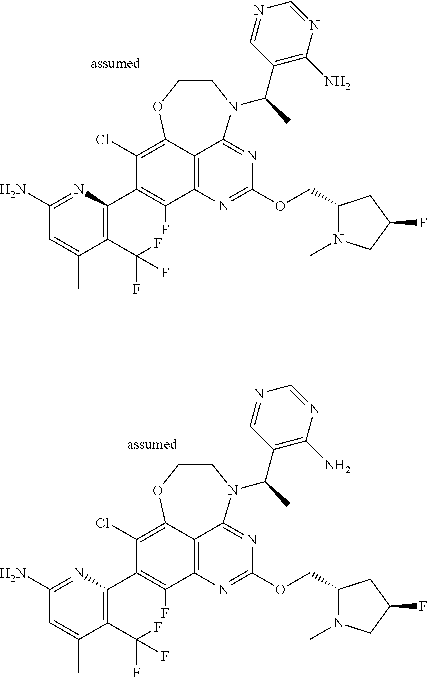

A solution of 5-((1R)-1-(9-(6-(bis(4-methoxybenzyl)amino)-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)-N-(4-methoxybenzyl)pyrimidin-4-amine (400.0 mg, crude) in 2,2,2-trifluoroacetic acid (5 mL) and trifluoromethanesulfonic acid (0.5 mL) was stirred at 25° C. for 16 hours. After completion, the solvent was removed under vacuum. The resulting solution was diluted with dichloromethane and adjust PH to 7 with saturated sodium sulfite. The resulting solution was extracted with dichloromethane. The organic layers were washed with brine, dried over anhydrous sodium sulfate and concentrated to afford crude product. The crude product was purified by Prep-HPLC with the following conditions: XBridge Prep OBD C18 Column, 30*150 mm, 5 μm; Mobile Phase A: water (10 mmol/L NH₄HCO₃), Mobile Phase B: acetonitrile; Flow rate: 60 mL/min; Gradient: 32% B to 50% B in 10.5 min; Wave Length: 220 nm; Wave Length: 220/254 nm; RT1(min): 6.7; RT2(min): 9.2; Number Of Runs: 4 to afford 5-((R)-1-((R)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyrimidin-4-amine (30.7 mg, 0.045 mmol, 19% yield) and 5-((R)-1-((S)-9-(6-amino-4-methyl-3-(trifluoromethyl)pyridin-2-yl)-8-chloro-10-fluoro-2-(((2S,4R)-4-fluoro-1-methylpyrrolidin-2-yl)methoxy)-5,6-dihydro-4H-[1,4]oxazepino[5,6,7-de]quinazolin-4-yl)ethyl)pyrimidin-4-amine (46.3 mg, 0.068 mmol, 28.8% yield). The stereo chemistry of title compounds was arbitrarily assigned.

Example 169a: ¹H NMR (300 MHz, DMSO-d₆, ppm) δ 8.38 (s, 1H), 8.30 (s, 1H), 6.81 (s, 2H), 6.78 (s, 2H), 6.48 (s, 1H), 6.16 (q, J=6.7 Hz, 1H), 5.19 (d, J=56.3 Hz, 1H), 4.56-4.18 (m, 4H), 3.71 (dd, J=15.5, 6.6 Hz, 1H), 3.60-3.35 (m, 2H), 3.32-3.29 (m, 1H), 3.03-2.89 (m, 1H), 2.40 (s, 3H), 2.43 (d, J=2.4 Hz, 3H), 2.24-2.07 (m, 1H), 2.02-1.78 (m, 1H), 1.60 (d, J=6.8 Hz, 3H). LCMS (ESI, m/z): 666.2 [M+H]⁺.

Example 169b: ¹H NMR (300 MHz, DMSO-d₆, ppm) δ 8.38 (s, 1H), 8.29 (s, 1H), 6.86 (s, 2H), 6.82 (s, 2H), 6.48 (s, 1H), 6.19 (q, J=6.7 Hz, 1H), 5.19 (d, J=55.9 Hz, 1H), 4.56 (dd, J=11.8, 6.6 Hz, 1H), 4.45-4.29 (m, 3H), 3.79 (dd, J=15.5, 6.3 Hz, 1H), 3.55-3.37 (m, 2H), 3.01-2.84 (m, 1H), 2.43-2.41 (m, 1H), 2.40 (s, 3H), 2.37 (d, J=2.2 Hz, 3H), 2.25-2.05 (m, 1H), 2.04-1.79 (m, 1H), 1.62 (d, J=6.8 Hz, 3H). LCMS (ESI, m/z): 666.2 [M+H]⁺.

Example 170a & 170b: 5-((R)-1-((R)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrimidin-4-amine

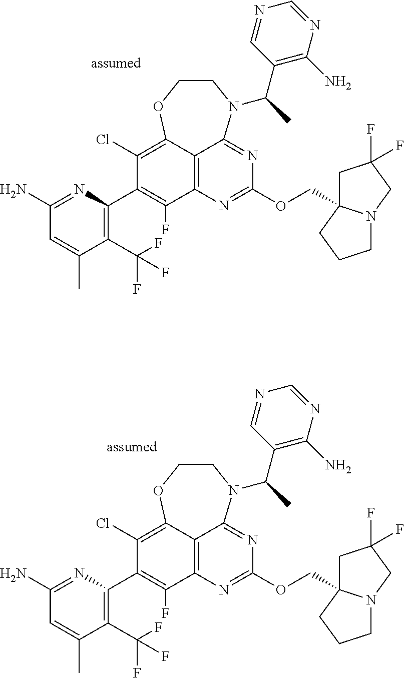

### Synthetic Route

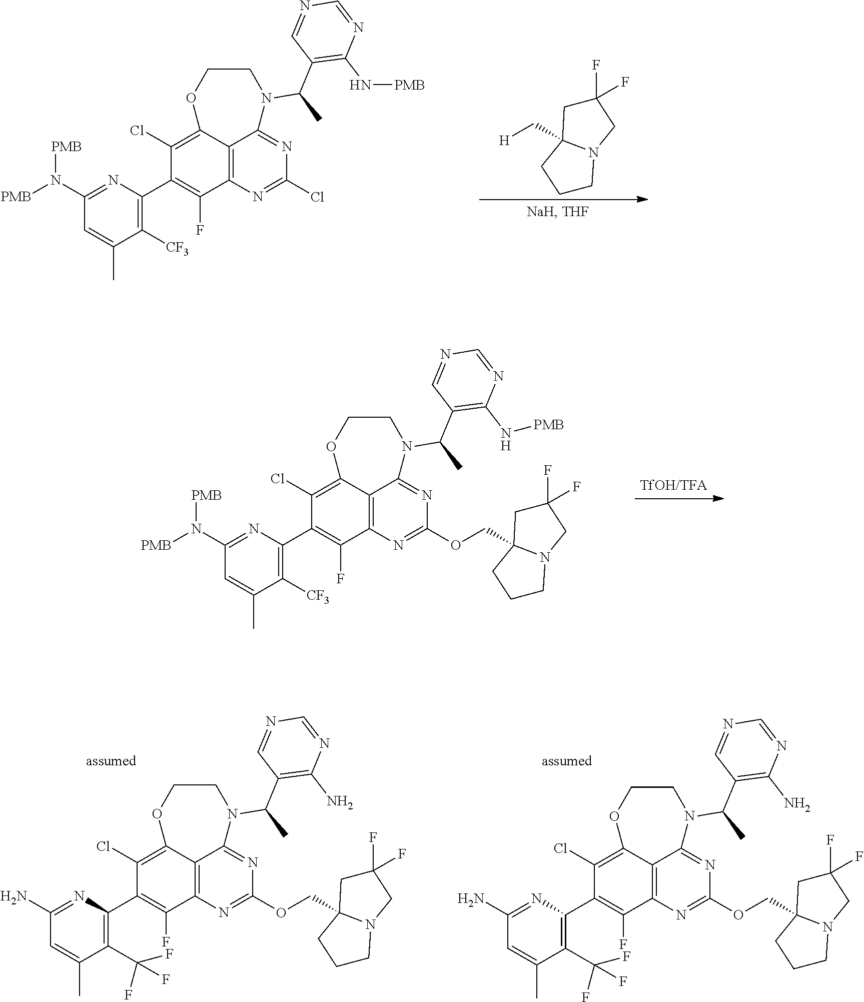

Step 1: 5-((1R)-1-(9-(6-(bis(4-methoxybenzyl)amino)-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine

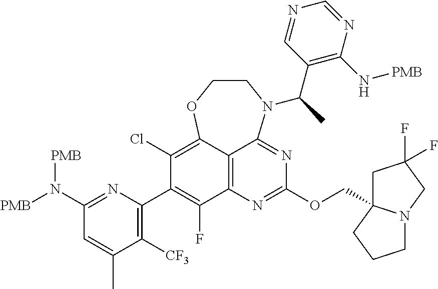

Under nitrogen, to a solution of [(8S)-6,6-difluoro-2,3,5,7-tetrahydro-1H-pyrrolizin-8-yl]methanol (171.5 mg, 0.97 mmol) in tetrahydrofuran (4 mL) was added sodium hydride (64.5 mg, 1.61 mmol, 60% dispersion in mineral oil), the mixture was stirred at 0° C. for 10 minutes. Then 5-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrimidin-4-amine (300.0 mg, 0.32 mmol) in tetrahydrofuran (1 mL) was added, the resulting solution was stirred for 1.5 hours at room temperature. After completion, the reaction was quenched with saturated ammonium chloride solution. The resulting solution was extracted with ethyl acetate. The organic layers were dried over anhydrous sodium sulfate and concentrated. The crude product would be directly used in the next step without purification. LC-MS: (ESI, m/z): 1070.4 [M+H]⁺

Step 2: 5-((R)-1-((R)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrimidin-4-amine & 5-((R)-1-((S)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrimidin-4-amine

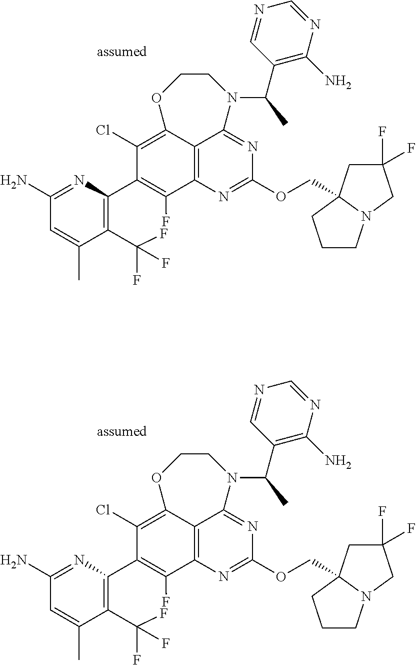

A mixture of 5-((1R)-1-(9-(6-(bis(4-methoxybenzyl)amino)-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)-N-(4-methoxybenzyl)pyrimidin-4-amine (450.0 mg, 0.42 mmol) in trifluoroacetic acid (5 mL) and trifuoromethanesulfonic acid (0.5 mL) was stirred at room temperature for 16 hours. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (92:8) to afford crude product. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 17% B to 42% B in 9 min, 42% B; Wave Length: 254/220 nm; RT1(min): 8.9 to afford 5-((R)-1-((R)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrimidin-4-amine (11.1 mg, 0.02 mmol, 3.6% yield) and 5-((R)-1-((S)-9-(6-amino-4-methyl-3-(trifluoromethyl)pyridin-2-yl)-8-chloro-2-(((S)-2,2-difluorotetrahydro-1H-pyrrolizin-7a(5H)-yl)methoxy)-10-fluoro-5,6-dihydro-4H-[1,4]oxazepino[5,6,7-de]quinazolin-4-yl)ethyl)pyrimidin-4-amine (14.3 mg, 0.02 mmol, 4.7% yield). The stereo chemistry of title compounds was arbitrarily assigned.

Example 170a: ¹H NMR (400 MHz, Methanol-d₄, ppm) δ 8.39 (s, 1H), 8.32 (s, 1H), 6.59 (s, 1H), 6.49 (q, J=6.9 Hz, 1H), 4.57-4.43 (m, 1H), 4.39-4.23 (m, 3H), 3.82-3.70 (m, 1H), 3.60-3.50 (m, 1H), 3.50-3.36 (m, 1H), 3.23-3.06 (m, 2H), 2.91-2.80 (m, 1H), 2.68-2.53 (m, 1H), 2.43 (d, J=1.2 Hz, 3H), 2.40-2.26 (m, 1H), 2.22-2.11 (m, 1H), 2.06-1.83 (m, 3H), 1.70 (d, J=6.9 Hz, 3H). LC-MS: (ESI, m/z): 710.2 [M+H]⁺.

Example 170b: ¹H NMR (400 MHz, Methanol-d₄, ppm) δ 8.39 (s, 1H), 8.32 (s, 1H), 6.58 (s, 1H), 6.50 (q, J=6.9 Hz, 1H), 4.54 (dd, J=12.5, 6.0 Hz, 1H), 4.40-4.23 (m, 3H), 3.80 (dd, J=15.6, 6.7 Hz, 1H), 3.58-3.36 (m, 2H), 3.22-3.06 (m, 2H), 2.92-2.79 (m, 1H), 2.69-2.53 (m, 1H), 2.43 (d, J=1.2 Hz, 3H), 2.41-2.26 (m, 1H), 2.22-2.12 (m, 1H), 2.08-1.83 (m, 3H), 1.70 (d, J=7.0 Hz, 3H). LC-MS: (ESI, m/z): 710.2 [M+H]⁺.

Example 171a & 171b: 3-((R)-1-((R)-9-(6-amino-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine

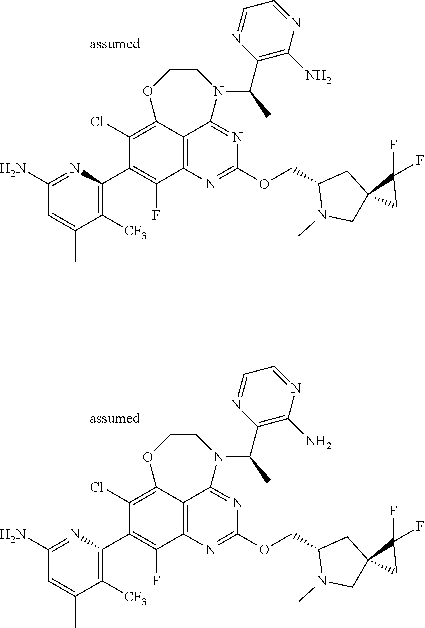

### Synthetic Route

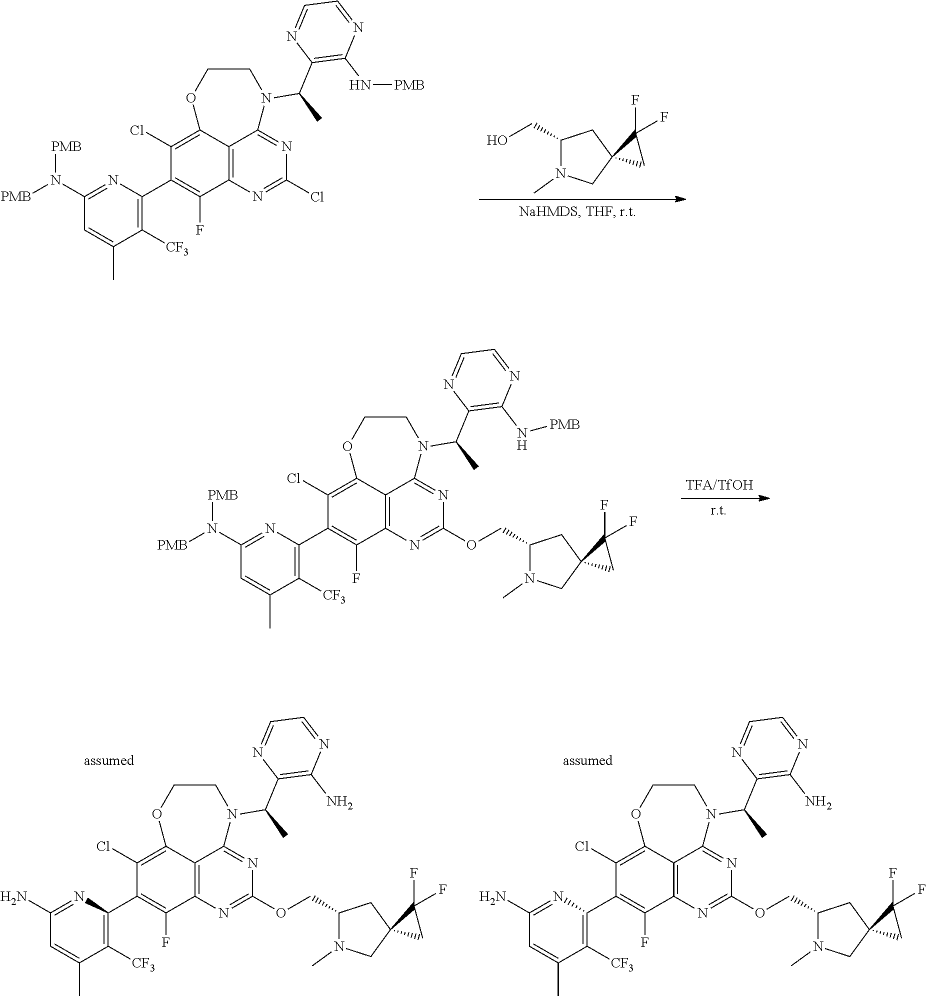

Step 1: 3-((1R)-1-(9-(6-(bis(4-methoxybenzyl)amino)-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)-N-(4-methoxybenzyl)pyrazin-2-amine

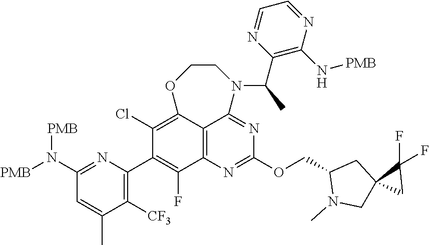

Under nitrogen, to a solution of ((3S,6S)-1,1-difluoro-5-methyl-5-azaspiro[2.4]heptan-6-yl)methanol (71.5 mg, 0.40 mmol) in tetrahydrofuran (2 mL) was added sodium bis(trimethylsilyl)amide (0.67 mL, 0.67 mmol, 1 M in tetrahydrofuran) at 0° C. and stirred at 25° C. for 1 hour. Under nitrogen, the reaction mixture was added to a solution of 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (250.0 mg, 0.27 mmol) in tetrahydrofuran (5 mL) at 25° C. and stirred at 25° C. for 1 hour. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with dichloromethane/ethyl acetate (1:1) to afford 3-((1R)-1-(9-(6-(bis(4-methoxybenzyl)amino)-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (130.2 mg, 0.11 mmol, 41.1% yield) as a light orange solid. LC-MS: (ESI, m/z): 1070.4 [M+H]⁺

Step 2: 3-((R)-1-((R)-9-(6-amino-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine

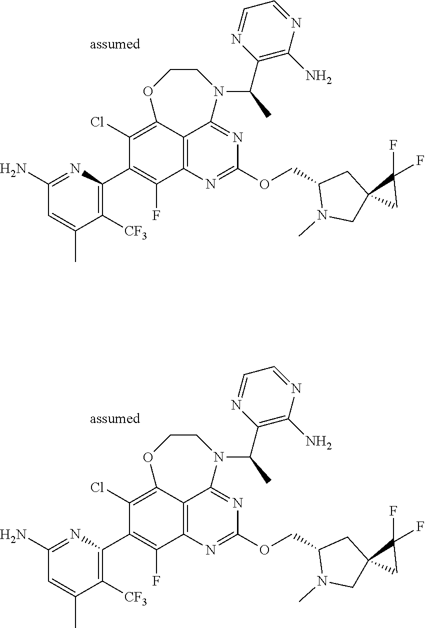

A solution of 3-((1R)-1-(9-(6-(bis(4-methoxybenzyl)amino)-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (130.0 mg, 0.11 mmol) in trifluoroacetic acid (5 mL) and trifluoromethanesulfonic acid (0.5 mL) was stirred for 1 hour at 25° C. After completion, the reaction mixture was diluted with dichloromethane, adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: XBridge Prep OBD C18 Column, 30*150 mm, 5 μm; Mobile Phase A: Water (10 mmol/L NH₄HCO₃), Mobile Phase B: acetonitrile; Flow rate: 60 mL/min; Gradient: 41% B to 62% B in 9 min, 62% B; Wave Length: 254/220 nm; RT1(min): 8.18 to afford 3-((R)-1-((R)-9-(6-amino-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine (14.7 mg, 0.02 mmol, 18.4% yield) and 3-((R)-1-((S)-9-(6-amino-4-methyl-3-(trifluoromethyl)pyridin-2-yl)-8-chloro-2-(((3S,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine (12.0 mg, 0.02 mmol, 14.8% yield). LC-MS: (ESI, m/z): 710.2 [M+H]⁺

Example 171a: ¹H NMR (300 MHz, DMSO-d₆, ppm) δ 7.95 (d, J=2.7 Hz, 1H), 7.78 (d, J=2.4 Hz, 1H), 6.81 (s, 2H), 6.48 (s, 1H), 6.35 (s, 2H), 6.29 (q, J=6.9 Hz, 1H), 4.58-4.47 (m, 1H), 4.43-4.25 (m, 3H), 3.92-3.77 (m, 1H), 3.65-3.53 (m, 1H), 2.94-2.69 (m, 2H), 2.60-2.51 (m, 1H), 2.42-2.27 (m, 6H), 2.26-2.11 (m, 1H), 1.81-1.68 (m, 1H), 1.65-1.33 (m, 5H). LC-MS: (ESI, m/z): 710.2 [M+H]⁺

Example 171b: ¹H NMR (300 MHz, DMSO-d₆, ppm) δ 7.87 (d, J=2.7 Hz, 1H), 7.70 (d, J=2.7 Hz, 1H), 6.75 (s, 2H), 6.38 (d, J=13.2 Hz, 3H), 6.20 (q, J=6.9 Hz, 1H), 4.69-4.52 (m, 1H), 4.44-4.21 (m, 3H), 3.96-3.80 (m, 1H), 3.69-3.51 (m, 1H), 2.88-2.64 (m, 2H), 2.56-2.46 (m, 1H), 2.35-2.22 (m, 6H), 2.21-2.03 (m, 1H), 1.79-1.62 (m, 1H), 1.59-1.25 (m, 5H). LC-MS: (ESI, m/z): 710.2 [M+H]⁺

Example 172a & 172b: 3-((R)-1-((R)-9-(6-amino-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine

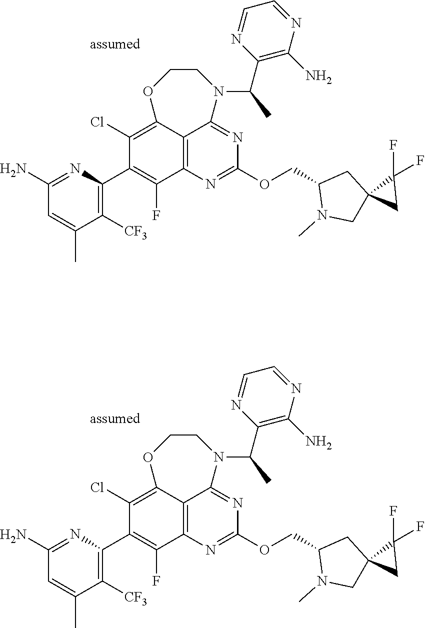

### Synthetic Route

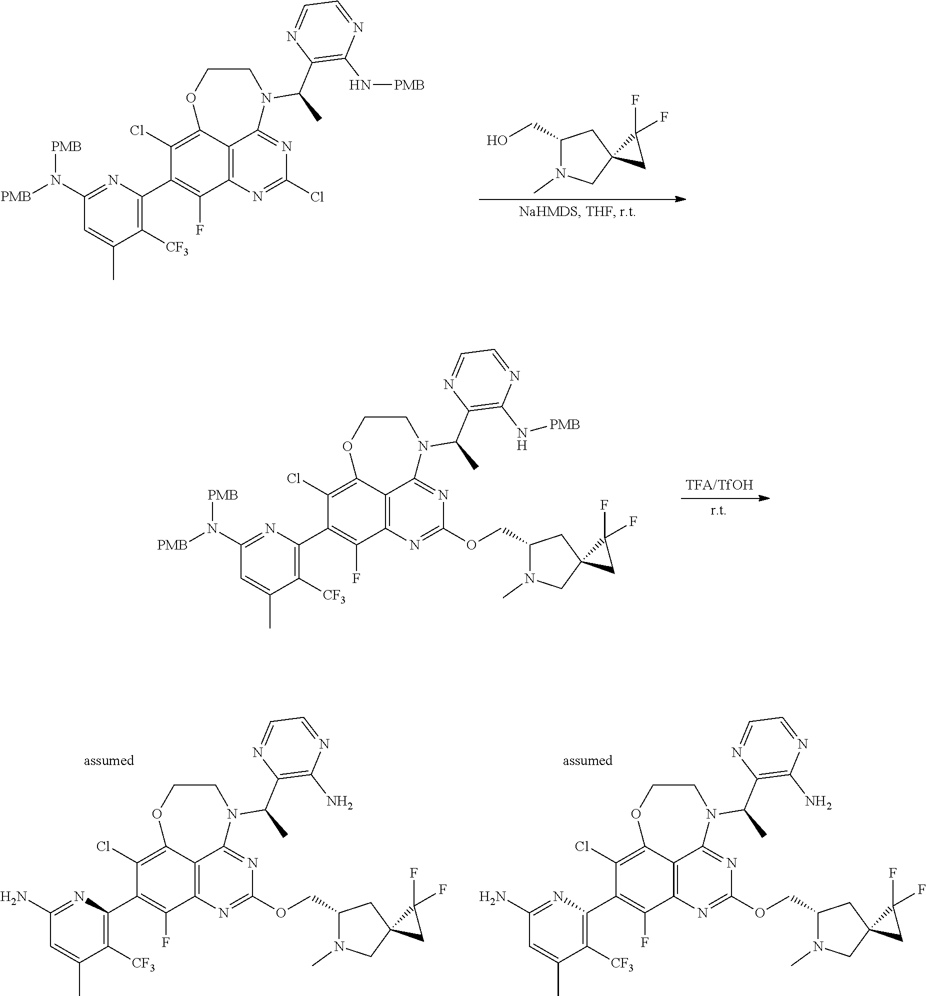

Step 1: 3-((1R)-1-(9-(6-(bis(4-methoxybenzyl)amino)-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)-N-(4-methoxybenzyl)pyrazin-2-amine

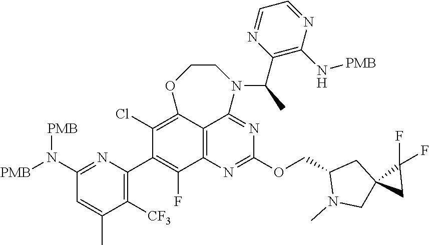

Under nitrogen, to a solution of ((3R,6S)-1,1-difluoro-5-methyl-5-azaspiro[2.4]heptan-6-yl)methanol (77.2 mg, 0.44 mmol) in tetrahydrofuran (2 mL) was added sodium bis(trimethylsilyl)amide (0.73 mL, 0.73 mmol, 1 M in tetrahydrofuran) at 0° C. and stirred at 25° C. for 1 hour. Under nitrogen, the reaction mixture was added to a solution of 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (270.0 mg, 0.29 mmol) in tetrahydrofuran (3 mL) at 25° C. and stirred at 25° C. for 1 hour. After completion, the reaction was quenched with ammonium chloride saturated solution, concentrated under vacuum, diluted with ethyl acetate, washed with water, dried over anhydrous sodium sulfate and concentrated under vacuum. The residue was purified by flash chromatography on silica gel eluting with dichloromethane/ethyl acetate (1:1) to afford 3-((1R)-1-(9-(6-(bis(4-methoxybenzyl)amino)-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (180.2 mg, 0.14 mmol, 48.6% yield) as a light orange solid. LC-MS: (ESI, m/z): 1070.4 [M+H]⁺

Step 2: 3-((R)-1-((R)-9-(6-amino-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine

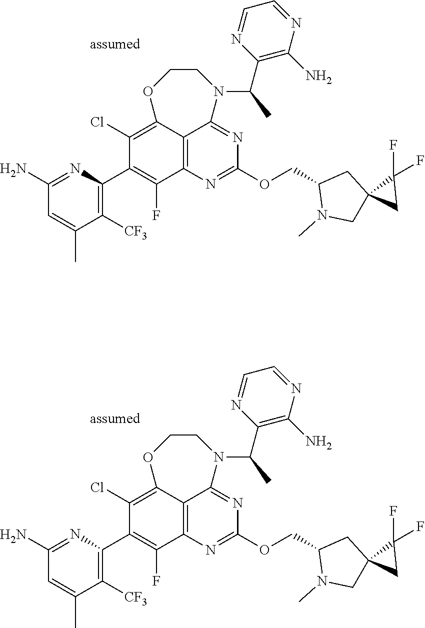

A solution of 3-((1R)-1-(9-(6-(bis(4-methoxybenzyl)amino)-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)-N-(4-methoxybenzyl)pyrazin-2-amine (150.0 mg, 0.14 mmol) in trifluoroacetic acid (6 mL) and trifluoromethanesulfonic acid (0.6 mL) was stirred for 1 hour at 25° C. After completion, the reaction mixture was diluted with dichloromethane, adjusted to pH >7 with sodium bicarbonate saturated solution, extracted with dichloromethane and the organic layer was combined. The organic layer was dried over anhydrous sodium sulfate and concentrated under vacuum. The residue was purified by flash chromatography on silica gel eluting with dichloromethane/methanol (8:1) to afford product. The product was further purified by Prep-HPLC with the following conditions: Column: Xselect CSH C18 OBD Column 30*150 mm 5 μm, n; Mobile Phase A: water (0.1% FA), Mobile Phase B: acetonitrile; Flow rate: 60 mL/min; Gradient: 23% B to 37% B in 10 min, 37% B: Wave Length: 254/220 nm; RT1(min): 8, 10 to afford 3-((R)-1-((R)-9-(6-amino-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine (15.0 mg, 0.02 mmol, 14.1% yield) and 3-((R)-1-((S)-9-(6-amino-4-methyl-3-(trifluoromethyl)pyridin-2-yl)-8-chloro-2-(((3R,6S)-1,1-difluoro-5-methyl-5-azaspiro[2.4]heptan-6-yl)methoxy)-10-fluoro-5,6-dihydro-4H-[1,4]oxazepino[5,6,7-de]quinazolin-4-yl)ethyl)pyrazin-2-amine (29.1 mg, 0.04 mmol, 27.3% yield). LC-MS: (ESI, m/z): 710.2 [M+H]⁺

Example 172a: ¹H NMR (300 MHz, DMSO-d₆, ppm) δ 7.96 (d, J=2.7 Hz, 1H), 7.79 (d, J=2.7 Hz, 1H), 6.82 (s, 2H), 6.49 (s, 1H), 6.37-6.18 (m, 3H), 4.57-4.19 (m, 4H), 3.90-3.76 (m, 1H), 3.63-3.49 (m, 1H), 3.04-2.81 (m, 2H), 2.46-2.39 (m, 1H), 2.39-2.31 (m, 6H), 2.10-1.95 (m, 1H), 1.93-1.79 (m, 1H), 1.59 (d, J=6.9 Hz, 3H), 1.52-1.34 (m, 2H). LC-MS: (ESI, m/z): 710.2 [M+H]⁺

Example 172b: ¹H NMR (300 MHz, DMSO-d₆, ppm) δ 7.95 (d, J=2.7 Hz, 1H), 7.78 (d, J=2.7 Hz, 1H), 6.82 (s, 2H), 6.49 (s, 1H), 6.41 (s, 2H), 6.29 (q, J=6.6 Hz, 1H), 4.66-4.53 (m, 1H), 4.45-4.20 (m, 3H), 3.98-3.83 (m, 1H), 3.68-3.56 (m, 1H), 3.10-2.83 (m, 2H), 2.45-2.26 (m, 7H), 2.12-1.98 (m, 1H), 1.95-1.75 (m, 1H), 1.60 (d, J=6.9 Hz, 3H), 1.53-1.32 (m, 2H). LC-MS: (ESI, m/z): 710.2 [M+H]⁺

Example 173a & 173b: 3-((R)-1-((R)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrazin-2-amine

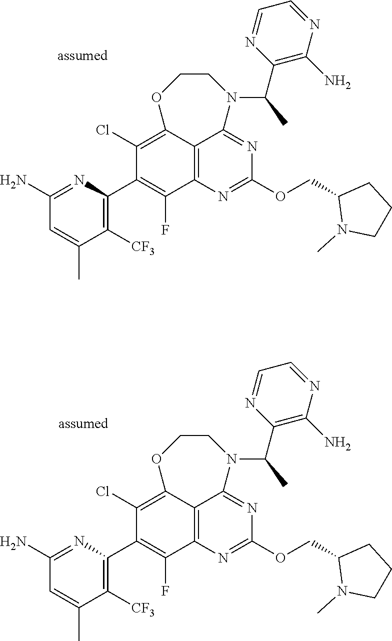

### Synthetic Route

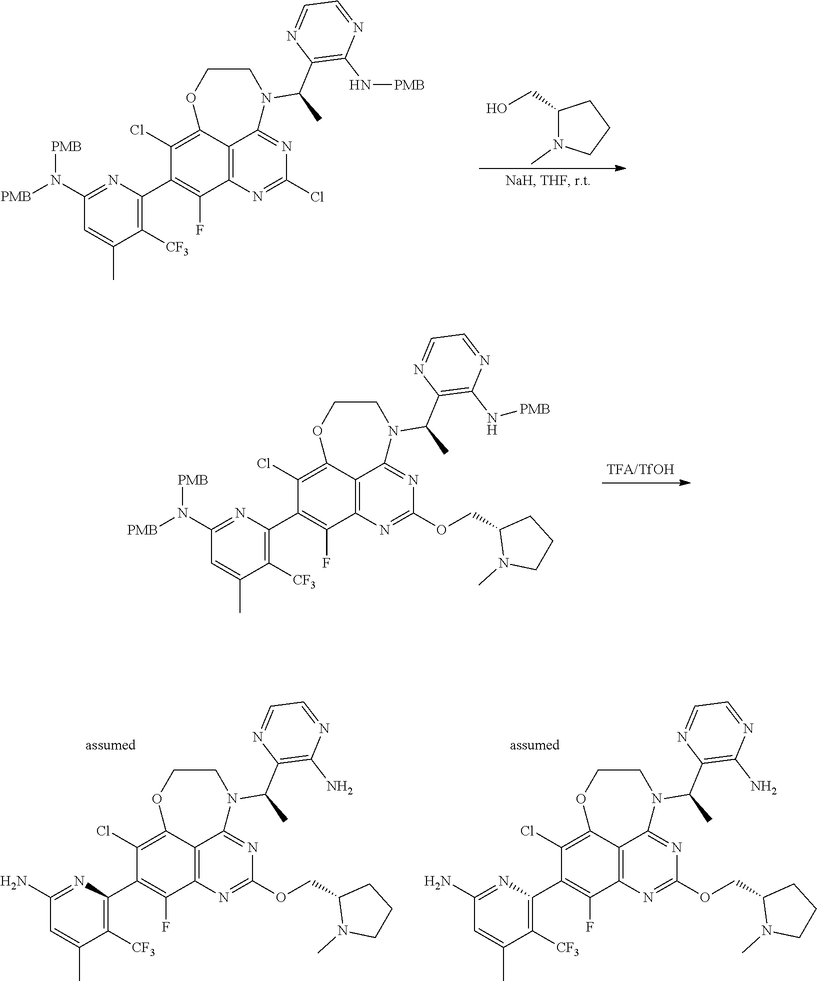

Step 1: 3-((1R)-1-(9-(6-(bis(4-methoxybenzyl)amino)-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)-N-(4-methoxybenzyl)pyrazin-2-amine

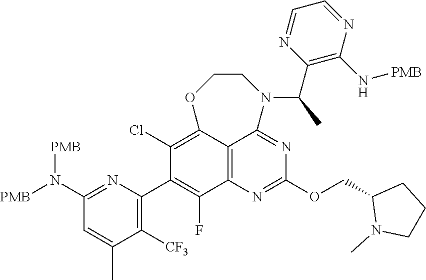

To a solution of N-methyl-I-prolinol (61.9 mg, 0.54 mmol) in tetrahydrofuran (4 mL) was added sodium hydride (32.2 mg, 0.81 mmol, 60% dispersion in mineral oil), the mixture was stirred at 0° C. for 10 minutes. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine (280 mg, 0.25 mmol, 92.1% yield) was added, the mixture was stirred for 1 hour at room temperature. After completion, the reaction was quenched with saturated ammonium chloride solution. The resulting solution was extracted with ethyl acetate. The organic layers were washed with brine, dried over anhydrous sodium sulfate and concentrated. The crude product would be directly used in the next step without purification. LC-MS: (ESI, m/z): 1008.4 [M+H]⁺

Step 2: 3-((R)-1-((R)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrazin-2-amine

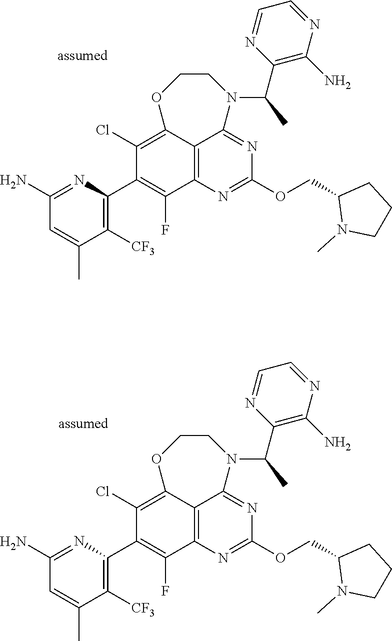

A mixture of 3-((1R)-1-(9-(6-(bis(4-methoxybenzyl)amino)-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)-N-(4-methoxybenzyl)pyrazin-2-amine (280.0 mg, 0.28 mmol) in trifluoroacetic acid (5 mL) and trifluoromethanesulfonic acid (0.5 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (93:7) to afford crude product. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 40% B to 60% B in 9.5 min; Wave Length: 254 nm; RT1(min): 6.2, 8.2 (min): Number Of Runs: 2 to afford 3-((R)-1-((R)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrazin-2-amine (14.8 mg, 0.02 mmol, 8.1% yield) and 3-((R)-1-((S)-9-(6-amino-4-methyl-3-(trifluoromethyl)pyridin-2-yl)-8-chloro-10-fluoro-2-(((S)-1-methylpyrrolidin-2-yl)methoxy)-5,6-dihydro-4H-[1,4]oxazepino[5,6,7-de]quinazolin-4-yl)ethyl)pyrazin-2-amine (17.4 mg, 0.03 mmol, 9.6% yield). The stereo chemistry of title compounds was arbitrarily assigned.

Example 173a: ¹H NMR (300 MHz, DMSO-d₆, ppm) δ 7.95 (d, J=2.7 Hz, 1H), 7.79 (d, J=2.7 Hz, 1H), 6.81 (s, 2H), 6.48 (s, 1H), 6.43-6.22 (m, 3H), 4.53 (dd, J=12.3, 6.3 Hz, 1H), 4.44-4.25 (m, 2H), 4.18 (dd, J=10.8, 6.3 Hz, 1H), 3.84 (dd, J=15.7, 6.5 Hz, 1H), 3.58 (dd, J=15.5, 6.7 Hz, 1H), 3.11-2.85 (m, 1H), 2.60-2.52 (m, 1H), 2.37-2.36 (m, 6H), 2.25-2.11 (m, 1H), 2.03-1.86 (m, 1H), 1.81-1.62 (m, 3H), 1.58 (d, J=6.8 Hz, 3H). LC-MS: (ESI, m/z): 648.3 [M+H]⁺.

Example 173b: ¹H NMR (300 MHz, DMSO-d₆, ppm) δ 7.94 (d, J=2.7 Hz, 1H), 7.78 (d, J=2.7 Hz, 1H), 6.81 (s, 2H), 6.48 (s, 1H), 6.41 (s, 2H), 6.29 (q, J=6.7 Hz, 1H), 4.69-4.52 (m, 1H), 4.45-4.32 (m, 1H), 4.32-4.15 (m, 2H), 3.92 (dd, J=15.7, 6.2 Hz, 1H), 3.63 (dd, J=15.6, 6.9 Hz, 1H), 3.03-2.88 (m, 1H), 2.59-2.52 (m, 1H), 2.36 (d, J=3.6 Hz, 3H), 2.35 (s, 3H), 2.25-2.12 (m, 1H), 2.03-1.87 (m, 1H), 1.77-1.62 (m, 3H), 1.59 (d, J=6.8 Hz, 3H). LC-MS: (ESI, m/z): 648.2 [M+H]⁺.

Example 174a & 174b: 3-((R)-1-((R)-9-(6-amino-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)pyrazin-2-amine

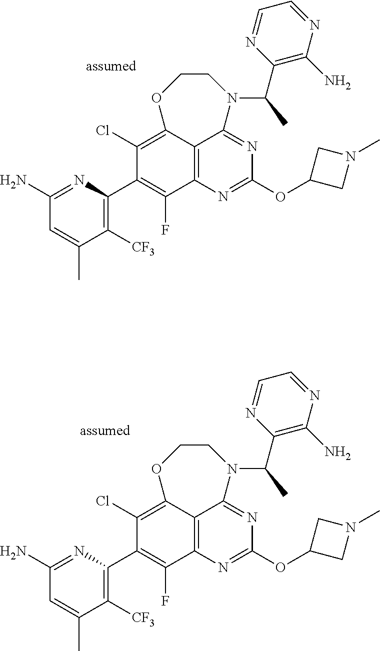

### Synthetic Route

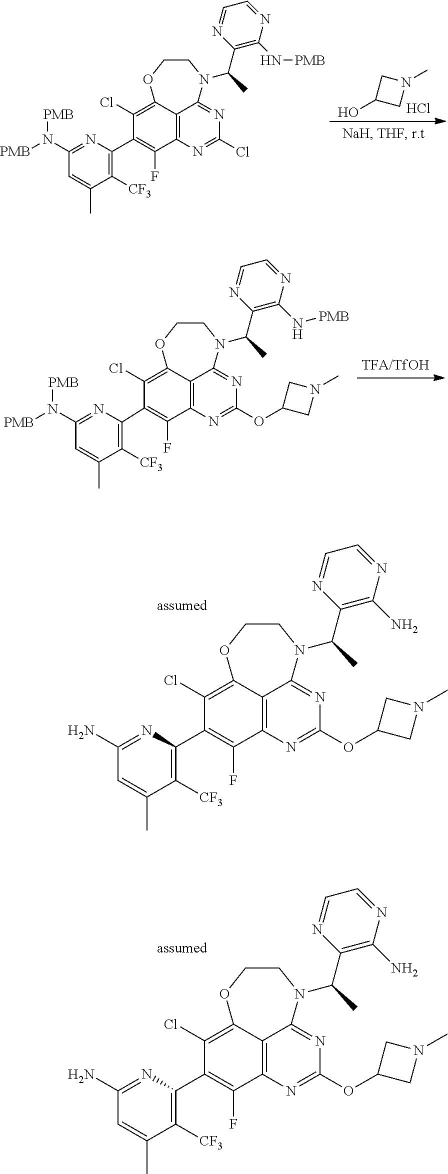

Step 1: 3-((1R)-1-(9-(6-(bis(4-methoxybenzyl)amino)-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)-N-(4-methoxybenzyl)pyrazin-2-amine

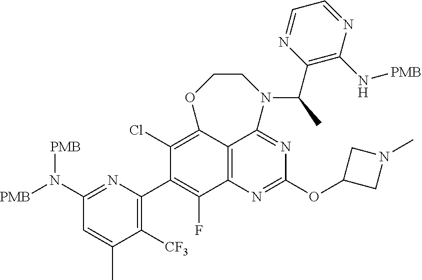

To a mixture of 3-hydroxy-1-methylazetidine hydrochloride (66.5 mg, 0.54 mmol) in tetrahydrofuran (3 mL) was added sodium hydride (64.5 mg, 1.61 mmol), the mixture was stirred for 10 minutes at 0° C. Then 3-((1R)-1-(9-(6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyrazin-2-amine [(4-methoxyphenyl)methyl]-3-[rac-(1R)-1-[7-[6-[bis[(4-methoxyphenyl)methyl]amino]-4-methyl-3-(trifluoromethyl)-2-pyridyl]-3,8-dichloro-6-fluoro-10-oxa-2,4,13-triazatricyclo[7.4.1.05,14]tetradeca-1,3,5(14),6,8-pentaen-13-yl]ethyl]pyrazin-2-amine (250.0 mg, 0.27 mmol) was added, stirred for 1 hour at room temperature. After completion, the reaction was quenched with saturated ammonium chloride solution. The resulting solution was diluted with water and extracted with ethyl acetate. The organic layers were combined, washed with brine, dried over anhydrous sodium sulfate and concentrated. The crude product would be directly used in the next step without purification. LC-MS: (ESI, m/z): 980.4 [M+H]⁺

Step 2: 3-((R)-1-((R)-9-(6-amino-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)pyrazin-2-amine & 3-((R)-1-((S)-9-(6-amino-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)pyrazin-2-amine

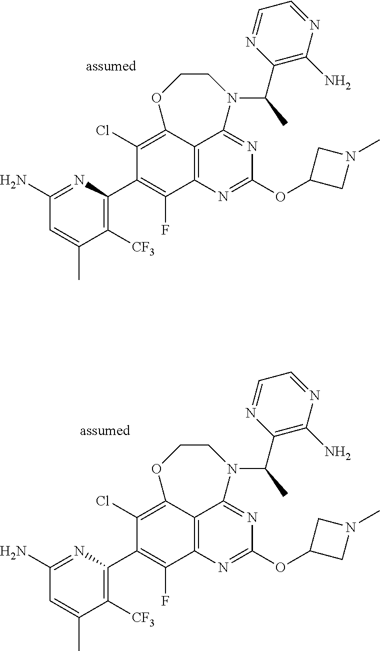

A mixture of 3-((1R)-1-(9-(6-(bis(4-methoxybenzyl)amino)-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)-N-(4-methoxybenzyl)pyrazin-2-amine (320.0 mg, 0.33 mmol) in trifluoroacetic acid (3 mL) and trifluoromethanesulfonic acid (0.3 mL) was stirred at room temperature for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The residue was purified by flash chromatography on silica gel eluting with dichloromethane/methanol (92:8) to afford crude product. The crude product was purified by Prep-HPLC with the following conditions: Column: XBridge Prep OBD C18 Column, 30*150 mm, 5 μm; Mobile Phase A: Water (10 mmol/L NH₄HCO₃), Mobile Phase B: ACN; Flow rate: 60 mL/min; Gradient: 35% B to 49% B in 10.5 min; Wave Length: 254 nm; RT1(min): 6, 8(min): Number Of Runs: 2 to afford 3-((R)-1-((R)-9-(6-amino-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)pyrazin-2-amine (9.8 mg, 0.02 mmol, 4.7% yield) and 3-((R)-1-((S)-9-(6-amino-4-methyl-3-(trifluoromethyl)pyridin-2-yl)-8-chloro-10-fluoro-2-((1-methylazetidin-3-yl)oxy)-5,6-dihydro-4H-[1,4]oxazepino[5,6,7-de]quinazolin-4-yl)ethyl)pyrazin-2-amine (16.4 mg, 0.03 mmol, 7.7% yield). The stereo chemistry of title compounds was arbitrarily assigned.

Example 174a: ¹H NMR (300 MHz, DMSO-da, ppm) δ 7.95 (d, J=2.7 Hz, 1H), 7.78 (d, J=2.7 Hz, 1H), 6.79 (s, 2H), 6.46 (s, 1H), 6.32-6.19 (m, 3H), 5.22-5.06 (m, 1H), 4.49 (dd, J=11.8, 6.6 Hz, 1H), 4.34 (dd, J=12.0, 6.1 Hz, 1H), 3.85-3.58 (m, 3H), 3.52 (dd, J=15.8, 6.6 Hz, 1H), 3.08-2.90 (m, 2H), 2.35 (d, J=2.2 Hz, 3H), 2.27 (s, 3H), 1.56 (d, J=6.8 Hz, 3H). LC-MS: (ESI, m/z): 620.1 [M+H]⁺.

Example 174b: ¹H NMR (300 MHz, DMSO-d₆, ppm) δ 7.94 (d, J=2.7 Hz, 1H), 7.77 (d, J=2.7 Hz, 1H), 6.79 (s, 2H), 6.46 (s, 1H), 6.34 (s, 2H), 6.26 (q, J=6.6 Hz, 1H), 5.22-5.08 (m, 1H), 4.57 (dd, J=11.9, 6.6 Hz, 1H), 4.33 (dd, J=12.1, 6.2 Hz, 1H), 3.85 (dd, J=15.6, 6.3 Hz, 1H), 3.74-3.51 (m, 3H), 3.04-2.91 (m, 2H), 2.35 (d, J=2.3 Hz, 3H), 2.26 (s, 3H), 1.57 (d, J=6.8 Hz, 3H). LC-MS: (ESI, m/z): 620.1 [M+H]⁺.

Example 175: 4-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyridazin-3-amine

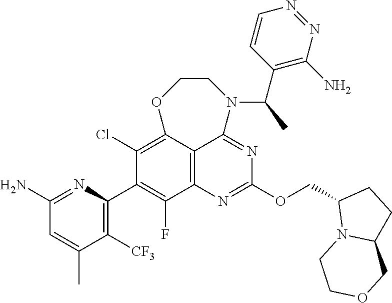

### Synthetic Route

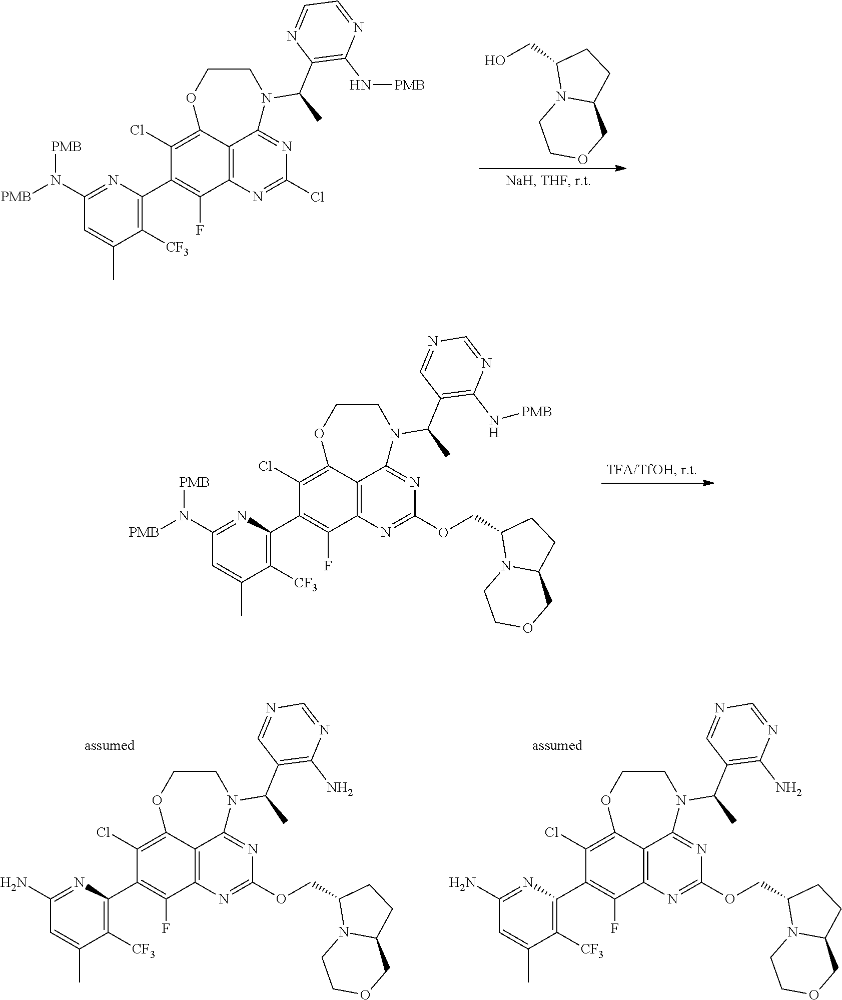

Step 1: 4-((R)-1-(9-((R)-6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyridazin-3-amine

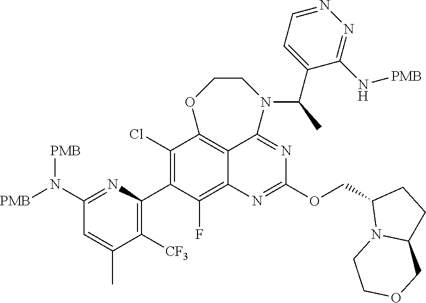

Under nitrogen, to a solution of ((6S,8aS)-hexahydro-1H-pyrrolo[2,1-c][1,4]oxazin-6-yl)methanol (405.7 mg, 2.58 mmol) in tetrahydrofuran (8 mL) was added sodium hydride (172.0 mg, 4.30 mmol, 60% dispersion in mineral oil), the mixture was stirred at 0° C. for 15 minutes. Then 4-((R)-1-(9-((R)-6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine (800.0 mg, 0.86 mmol) was added and stirred at 0° C. for 2 hours. After completion, the reaction was quenched with saturated ammonium chloride solution. The resulting solution was diluted with water and extracted with ethyl acetate. The organic layers were washed with brine, dried over anhydrous sodium sulfate and concentrated. The crude product would be directly used in the next step without purification. LC-MS: (ESI, m/z): 1050.4 [M+H]⁺.

Step 2: 4-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyridazin-3-amine

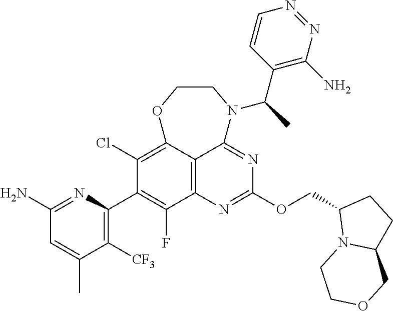

A solution of 4-((R)-1-(9-((R)-6-(bis(4-methoxybenzyl)amino)-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)-N-(4-methoxybenzyl)pyridazin-3-amine (840.0 mg, crude) in trifluoromethanesulfonic acid (1 mL) and 2,2,2-trifluoroacetic acid (10 mL) was stirred at 25° C. for 0.5 hour. After completion, the solvent was removed under vacuum. The resulting solution was diluted with dichloromethane and adjust PH to 7 with saturated sodium bicarbonate solution. The resulting solution was extracted with dichloromethane. The organic layers were washed with brine, dried over anhydrous sodium sulfate and concentrated. The crude product was purified by Prep-HPLC with the following conditions: XBridge Shield RP18 OBD Column, 30*150 mm, 5 μm; Mobile Phase A: water (10 mmol/L NH₄HCO₃), Mobile Phase B: acetonitrile; Flow rate: 60 mL/min; Gradient: 28% B to 42% B in 10 min; Wave Length: 254/220 nm; RT1: 10 min to afford 4-((R)-1-((R)-9-(6-amino-4-methyl-3-(trifluoromethyl)pyridin-2-yl)-8-chloro-10-fluoro-2-(((6S,8aS)-hexahydro-1H-pyrrolo[2,1-c][1,4]oxazin-6-yl)methoxy)-5,6-dihydro-4H-[1,4]oxazepino[5,6,7-de]quinazolin-4-yl)ethyl)pyridazin-3-amine (44.6 mg, 0.06 mmol, 20.1% yield). LC-MS: (ESI, m/z): 690.4 [M+H]⁺.

Example 175: ¹H NMR (300 MHz, Methanol-d₄, ppm) δ 8.54 (d, J=4.9 Hz, 1H), 7.54 (d, J=4.9, 0.9 Hz, 1H), 6.61 (s, 1H), 6.41 (q, J=6.9 Hz, 1H), 4.65-4.48 (m, 1H), 4.48-4.21 (m, 3H), 3.92-3.71 (m, 3H), 3.71-3.52 (m, 3H), 3.40-3.34 (m, 1H), 3.16-3.05 (m, 2H), 3.05-2.91 (m, 1H), 2.46 (d, J=2.2 Hz, 3H), 2.30-2.11 (m, 1H), 2.00-1.83 (m, 1H), 1.83-1.74 (m, 1H), 1.71 (d, J=6.9 Hz, 3H), 1.58-1.39 (m, 1H).

Example 176: 4-((R)-1-((R)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyridazin-3-amine

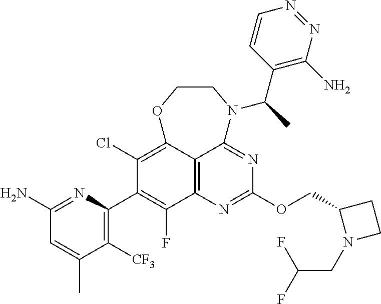

### Synthetic Route

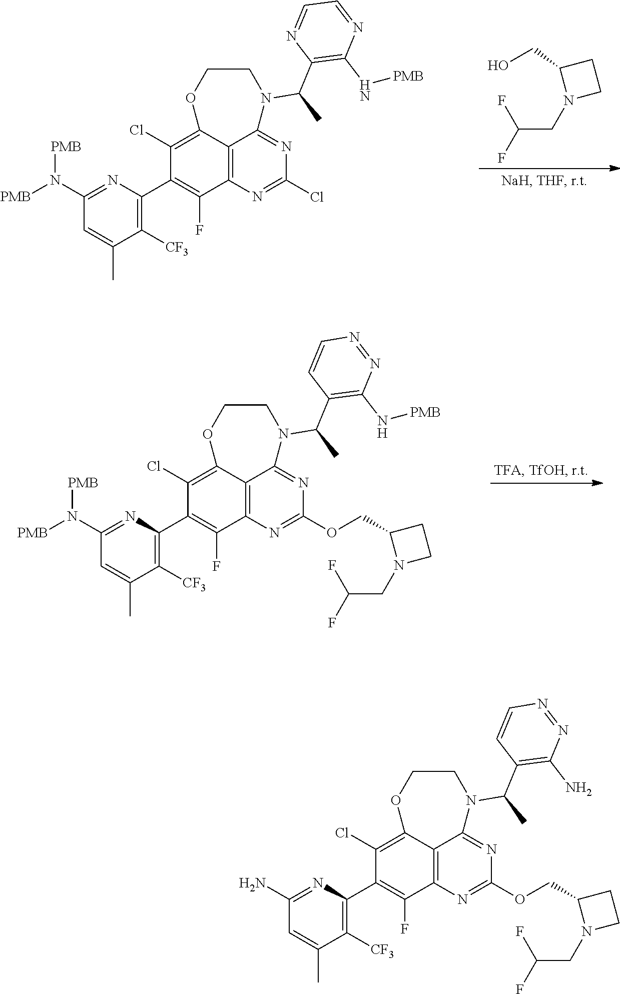

Step 1: 4-((R)-1-(9-((R)-6-(bis(4-methoxybenzyl)amino)-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)-N-(4-methoxybenzyl)pyridazin-3-amine

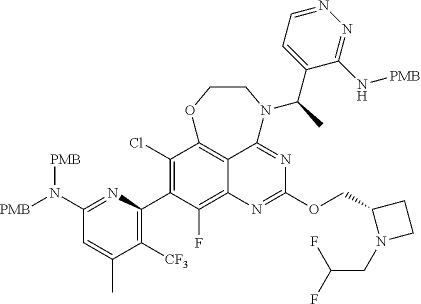

Under nitrogen, to a solution of (S)-(1-(2,2-difluoroethyl)azetidin-2-yl)methanol (536.4 mg, 3.55 mmol) in tetrahydrofuran (11 mL) was added sodium hydride (189.2 mg, 4.73 mmol, 60% dispersion in mineral oil), the mixture was stirred at 0° C. for 10 minutes. Then 4-((R)-1-(9-((R)-6-(bis(4-methoxybenzyl)amino)-4-methyl-3-(trifluoromethyl)pyridin-2-yl)-2,8-dichloro-10-fluoro-5,6-dihydro-4H-[1,4]oxazepino[5,6,7-de]quinazolin-4-yl)ethyl)-N-(4-methoxybenzyl)pyridazin-3-amine (1.1 g, 1.18 mmol) was added and stirred at 25° C. for 1 hour. After completion, the reaction was quenched with saturated ammonium chloride solution. The resulting solution was diluted with water and extracted with ethyl acetate. The organic layers were washed with brine, dried over anhydrous sodium sulfate and concentrated. The crude product would be directly used in the next step without purification. LC-MS: (ESI, m/z): 1044.4 [M+H]⁺.

Step 2: 4-((R)-1-(9-((R)-6-(bis(4-methoxybenzyl)amino)-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)-N-(4-methoxybenzyl)pyridazin-3-amine

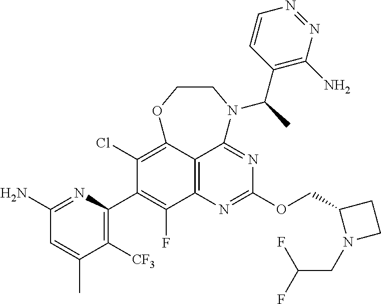

A solution of 4-((R)-1-(9-((R)-6-(bis(4-methoxybenzyl)amino)-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)-N-(4-methoxybenzyl)pyridazin-3-amine (1.1 g, crude) in 2,2,2-trifluoroacetic acid (20 mL) and trifluoromethanesulfonic acid (2.0 mL) was stirred at 25° C. for 1 hour. After completion, the solvent was removed under vacuum. The resulting solution was diluted with dichloromethane and adjust PH to 7 with saturated sodium sulfite. The resulting solution was extracted with dichloromethane. The organic layers were washed with brine, dried over anhydrous sodium sulfate, concentrated and purified by reverse phase chromatography (acetonitrile/0.1% NH₄Cl in water) to afford crude product. The crude product was purified by Prep-HPLC with the following conditions: XBridge Prep OBD C18 Column, 30*150 mm, 5 μm; Mobile Phase A: water (10 mmol/L NH4HCO₃), Mobile Phase B: acetonitrile; Flow rate: 60 mL/min; Gradient: 26% B to 51% B in 9 min; 254/220 nm; RT1: 8.88 min to afford 4-((R)-1-((R)-9-(6-amino-4-methyl-3-(trifluoromethyl)pyridin-2-yl)-8-chloro-2-(((S)-1-(2,2-difluoroethyl)azetidin-2-yl)methoxy)-10-fluoro-5,6-dihydro-4H-[1,4]oxazepino[5,6,7-de]quinazolin-4-yl)ethyl)pyridazin-3-amine (38.5 mg, 0.056 mmol, 13.3% yield). LC-MS: (ESI, m/z): 684.2 [M+H]⁺.

Example 176: ¹H NMR (300 MHz, Methanol-d₄, ppm) δ 8.56 (d, J=4.8 Hz, 1H), 7.56 (d, J=5.0, 1.0 Hz, 1H), 6.61 (s, 1H), 6.44 (q, J=6.9 Hz, 1H), 5.87 (tdd, J=56.4, 5.5, 3.2 Hz, 1H), 4.65-4.47 (m, 2H), 4.47-4.33 (m, 2H), 3.91-3.68 (m, 2H), 3.67-3.44 (m, 2H), 3.31-3.08 (m, 2H), 2.92-2.72 (m, 1H), 2.46 (d, J=2.1 Hz, 3H), 2.23-2.11 (m, 2H), 1.71 (d, J=6.9 Hz, 3H).

Example 501: 6-(8-Chloro-4-(2-(dimethylamino)ethyl)-5,6-dihydro-4H-[1,4]oxazepino[5,6,7-de]quinazolin-9-yl)-4-methyl-5-(trifluoromethyl)pyridin-2-amine

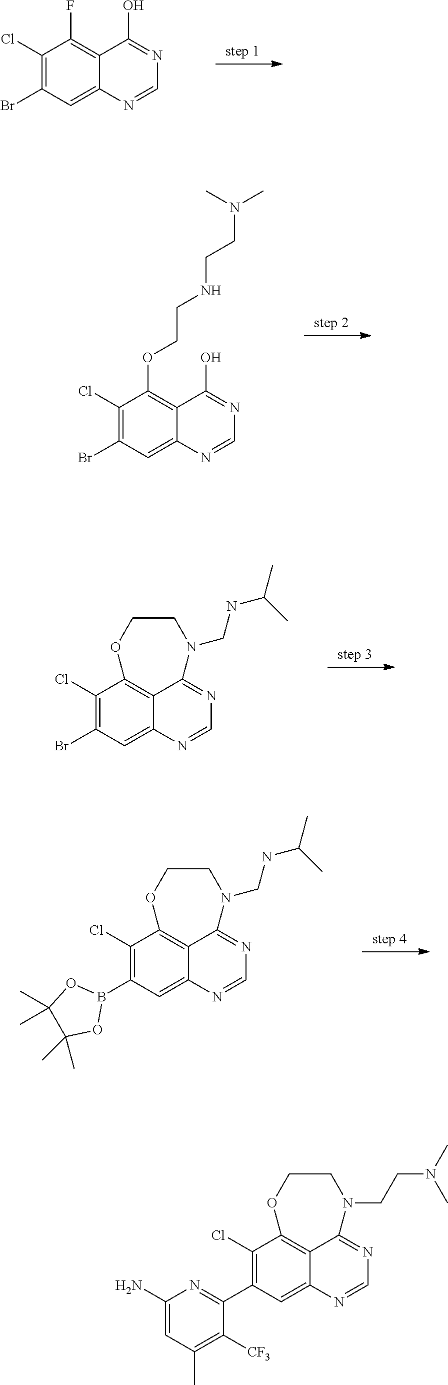 [SMILES: Oc1ncnc2cc(Br)c(Cl)c(F)c12 ; CN(C)CCNCCOc1c(Cl)c(Br)cc2ncnc(O)c12 ; CC(C)NCN1CCOc2c(Cl)c(Br)cc3ncnc1c23 ; CC(C)NCN1CCOc2c(Cl)c(B3OC(C)(C)C(C)(C)O3)cc3ncnc1c23 ; Cc1cc(N)nc(-c2cc3ncnc4c3c(c2Cl)OCCN4CCN(C)C)c1C(F)(F)F]

Step 1: 7-Bromo-6-chloro-5-(2-((2-(dimethylamino)ethyl)amino)ethoxy)quinazolin-4-ol

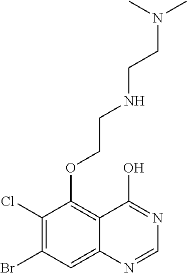 [SMILES: CN(C)CCNCCOc1c(Cl)c(Br)cc2ncnc(O)c12]

Under nitrogen, to a solution of 2-((2-(dimethylamino)ethyl)amino)ethan-1-ol (100 mg, 0.760 mmol) in tetrahydrofuran (2 mL) was added 60% NaH (181 mg, 4.55 mmol) at 0° C. The resulting solution was stirred at room temperature for 30 min. Then 7-bromo-6-chloro-5-fluoroquinazolin-4-ol (209 mg, 0.750 mmol) was added. Stirred at 65° C. for 1 hour. The reaction was quenched with acetic acid and concentrated under vacuum. The residue was purified by pre-packed C18 column (solvent gradient: 0-100% ACN in water (0.05% NH₄HCO₃)) to yield 145 mg (49% yield) of the title compound as a white solid. LC-MS: (ESI, m/z): [M+H]⁺=389.

Step 2: 2-(9-Bromo-8-chloro-5,6-dihydro-4H-[1,4]oxazepino[5,6,7-de]quinazolin-4-yl)-N,N-dimethylethan-1-amine

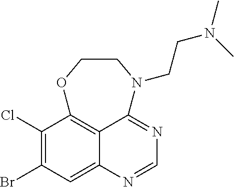 [SMILES: CN(C)CCN1CCOc2c(Cl)c(Br)cc3ncnc1c23]

Under nitrogen, a solution of 7-bromo-6-chloro-5-(2-((2-(dimethylamino)ethyl)amino)ethoxy)quinazolin-4-ol (125 mg, 0.320 mmol), PyBOP (251 mg, 0.480 mol) and DIPEA (415 mg, 0.800 mmol) in dichloromethane (2.5 mL) was stirred at room temperature for 24 hours. The resulting solution was diluted with DCM and washed with water and brine. The organic layer was dried over anhydrous sodium sulfate and concentrated under vacuum. The residue was purified by flash chromatography on silica gel (gradient: 0%-30% MeOH/DCM) to yield 113 mg (94% yield) of the title compound as a white solid. LC-MS: (ESI, m/z): [M+H]⁺=371.

Step 3: 2-(8-Chloro-9-(4,4,5,5-tetramethyl-1,3,2-dioxaborolan-2-yl)-5,6-dihydro-4H-[1,4]oxazepino[5,6,7-de]quinazolin-4-yl)-N,N-dimethylethan-1-amine

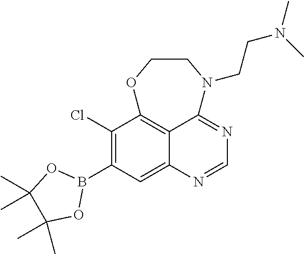

Under nitrogen, a mixture of 2-(9-bromo-8-chloro-5,6-dihydro-4H-[1,4]oxazepino[5,6,7-de]quinazolin-4-yl)-N,N-dimethylethan-1-amine (90.0 mg, 0.240 mmol), 4,4,4′,4′,5,5,5′,5′-octamethyl-2,2′-bi(1,3,2-dioxaborolane) (185 mg, 0.730 mmol), Pd(dppf)Cl₂ (35.0 mg, 0.050 mmol) and KOAc (71.0 mg, 0.730 mmol) in 1,4-dioxane (2 mL) was stirred at 80° C. for 5 hours. The solids were filtered off. The filtrate was diluted with EtOAc and extracted with water. The product was in the aqueous phase. The aqueous phase was concentrated under vacuum to yield 90 mg (crude) of the title compound as a yellow solid. LC-MS: (ESI, m/z): [M+H]⁺=337 (the corresponding boronic acid mass). The crude product was used for next step without further purification.

Step 4: 6-(8-Chloro-4-(2-(dimethylamino)ethyl)-5,6-dihydro-4H-[1,4]oxazepino[5,6,7-de]quinazolin-9-yl)-4-methyl-5-(trifluoromethyl)pyridin-2-amine

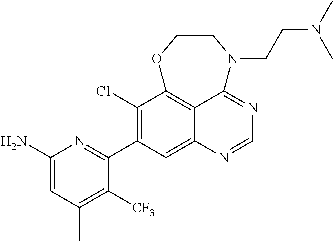 [SMILES: Cc1cc(N)nc(-c2cc3ncnc4c3c(c2Cl)OCCN4CCN(C)C)c1C(F)(F)F]

Under nitrogen, a solution of 2-(8-chloro-9-(4,4,5,5-tetramethyl-1,3,2-dioxaborolan-2-yl)-5,6-dihydro-4H-[1,4]oxazepino[5,6,7-de]quinazolin-4-yl)-N,N-dimethylethan-1-amine (80.0 mg, 0.190 mmol), 6-bromo-4-methyl-5-(trifluoromethyl)pyridin-2-amine (32.0 mg, 0.130 mmol), Pd(PPh₃)₄(14.0 mg, 0.0100 mmol) and K₂CO₃ (35.0 mg, 0.260 mmol) in acetonitrile (1.5 mL) and water (0.3 mL) was stirred at 100° C. for 1 hour. The mixture was partitioned between EtOAc and water. The organic phase was concentrated under vacuum. The residue was purified by Prep-HPLC (XBridge Prep OBD C18 Column, 30×150 mm Sum; Mobile Phase A: Water (10 mmol/L NH₄HCO₃), Mobile Phase B: ACN; Flow rate: 60 mL/min; Gradient: 30% B to 60% B in 7 min: 254 nm; R_{T1}: 6.33) to yield 15.4 mg (26% yield) of the title compound. LC-MS: (ESI, m/z): [M+H]⁺=467. ¹H NMR (300 MHz, DMSO-d₆, ppm) δ 8.40 (s, 1H), 7.14 (s, 1H), 6.75 (s, 2H), 6.45 (s, 1H), 4.69-4.54 (m, 2H), 4.11-3.81 (m, 4H), 2.57 (t, J=6.6 Hz, 2H), 2.36 (d, J=2.3 Hz, 3H), 2.23 (s, 6H).

Example 502: 2-((2-(9-(6-Amino-4-methyl-3-(trifluoromethyl)pyridin-2-yl)-8-chloro-5,6-dihydro-4H-[1,4]oxazepino[5,6,7-de]quinazolin-4-yl)ethyl)(methyl)amino)ethan-1-ol

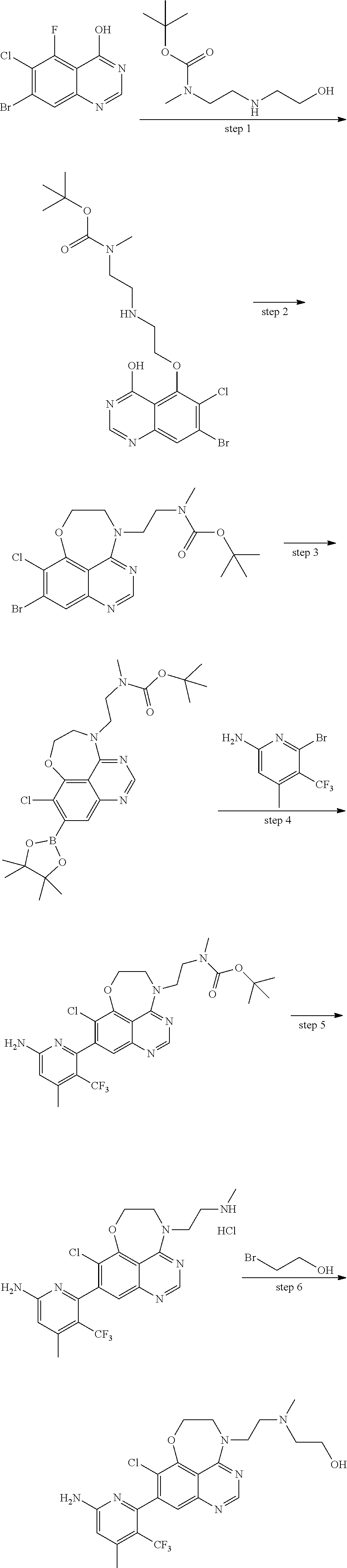

Step 1: tert-Butyl (2-((2-((7-bromo-6-chloro-4-hydroxyquinazolin-5 yl)oxy)ethyl)amino)ethyl)(methyl)carbamate

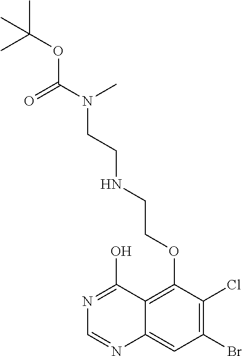 [SMILES: CN(CCNCCOc1c(Cl)c(Br)cc2ncnc(O)c12)C(=O)OC(C)(C)C]

To a solution of tert-butyl (2-((2-hydroxyethyl)amino)ethyl)(methyl)carbamate (682 mg, 3.13 mmol, intermediate 12) in tetrahydrofuran (20 mL) was added 60% NaH (480 mg, 12.5 mmol) at 0° C. The solution was warmed to room temperature and stirred for 0.5 hour. Then 7-bromo-6-chloro-5-fluoroquinazolin-4-ol (576 mg, 2.07 mmol) was added at room temperature and stirred at 65° C. for 2 hours. The reaction mixture was quenched with water and concentrated under vacuum. The residue was purified by flash chromatography on silica gel (gradient: 0%-10% MeOH/DCM) to yield 946 mg (95% yield) of the title compound as a yellow solid. LC-MS: (ESI, m/z): [M+H]⁺=475.1.

Step 2: tert-Butyl (2-(9-bromo-8-chloro-5,6-dihydro-4H-[1,4]oxazepino[5,6,7-de]quinazolin-4 yl)ethyl)(methyl)carbamate

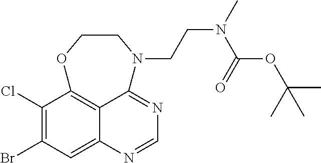 [SMILES: CN(CCN1CCOc2c(Cl)c(Br)cc3ncnc1c23)C(=O)OC(C)(C)C]

A solution of tert-butyl (2-((2-((7-bromo-6-chloro-4-hydroxyquinazolin-5 yl)oxy)ethyl)amino)ethyl)(methyl)carbamate (700 mg, 1.47 mmol), DIPEA (2.85 g, 22.0 mmol) and PyBOP (3.06 g, 5.88 mmol) in dichloromethane (15 mL) was stirred 8 hours at 25° C. The resulting mixture was partitioned between dichloromethane and water. The organic phase was concentrated under vacuum. The residue was purified by pre-packed C18 column (solvent gradient: 0%-100% MeOH in water (0.1% NH₄HCO₃)) to yield 367 mg (54% yield) of the title compound as a yellow solid. LC-MS: (ESI, m/z): [M+H]⁺=457.1

Step 3: tert-Butyl (2-(8-chloro-9-(4,4,5,5-tetramethyl-1,3,2-dioxaborolan-2-yl)-5,6-dihydro-4H-[1,4]oxazepino[5,6,7-de]quinazolin-4-yl)ethyl)(methyl)carbamate

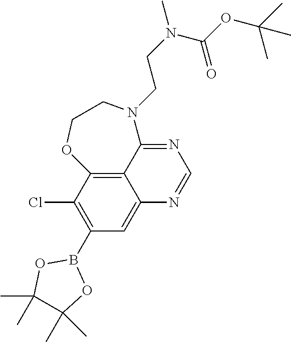 [SMILES: CN(CCN1CCOc2c(Cl)c(B3OC(C)(C)C(C)(C)O3)cc3ncnc1c23)C(=O)OC(C)(C)C]

Under nitrogen, a solution of tert-butyl (2-(9-bromo-8-chloro-5,6-dihydro-4H-[1,4]oxazepino[5,6,7-de]quinazolin-4 yl)ethyl)(methyl)carbamate (347 mg, 0.760 mmol), 4,4,4′,4′,5,5,5′,5′-octamethyl-2,2′-bi(1,3,2-dioxaborolane) (579 mg, 2.28 mmol), KOAc (148 mg, 1.52 mmol) and PdCl₂(dppf) (55.3 mg, 0.0700 mmol) in 1,4-dioxane (10 mL) was stirred at 100° C. for 2 hours. The solvent was stripped off under vacuum. The residue was treated with a mixture of petroleum and EtOAc (petroleum ether/EtOAc=10:1). The solid was filtered off. The filtrate was concentrated under reduced pressure to afford 385 mg (crude) the title compound. The crude was used for next step without further purification. LC-MS: (ESI, m/z): [M+H]⁺=505.

Step 4: tert-Butyl (2-(9-(6-amino-4-methyl-3-(trifluoromethyl)pyridin-2-yl)-8-chloro-5,6-dihydro-4H-[1,4]oxazepino[5,6,7-de]quinazolin-4-yl)ethyl)(methyl)carbamate

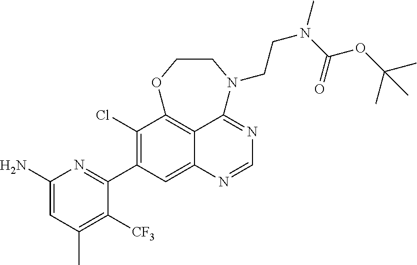 [SMILES: Cc1cc(N)nc(-c2cc3ncnc4c3c(c2Cl)OCCN4CCN(C)C(=O)OC(C)(C)C)c1C(F)(F)F]

Under nitrogen, a mixture of tert-butyl (2-(8-chloro-9-(4,4,5,5-tetramethyl-1,3,2-dioxaborolan-2-yl)-5,6-dihydro-4H-[1,4]oxazepino[5,6,7-de]quinazolin-4-yl)ethyl)(methyl)carbamate (385 mg, 0.760 mmol), 6-bromo-4-methyl-5-(trifluoromethyl)pyridin-2-amine (193 mg, 0.760 mmol), KF (88.4 mg, 1.52 mmol) and Pd(PPh₃)₂Cl₂ (53.6 mg, 0.0800 mmol) in acetonitrile (5 mL) and water (1 mL) was stirred for 1.5 h at 100° C. The mixture was cooled to ambient temperature and partitioned between EtOAc and water. The organic phase was concentrated under vacuum. The residue was purified by pre-packed C18 column (solvent gradient: 0%-100% ACN in water (0.05% TFA)) to yield 214 mg (50% yield) of the title compound as a light-yellow solid. LC-MS: (ESI, m/z): [M+H]⁺=553.3. ¹H NMR (400 MHz, DMSO-d₆, ppm) δ 8.84 (s, 1H), 7.23 (s, 1H), 6.85 (s, 2H), 6.50 (s, 1H), 4.97-4.69 (m, 2H), 4.55-4.41 (m, 1H), 4.27-3.92 (m, 3H), 3.61-3.51 (m, 2H), 2.86 (s, 3H), 2.36 (s, 3H), 1.17 (s, 9H).

Step 5: 6-(8-Chloro-4-(2-(methylamino)ethyl)-5,6-dihydro-4H-[1,4]oxazepino[5,6,7-de]quinazolin-9-yl)-4-methyl-5-(trifluoromethyl)pyridin-2-amine hydrochloride

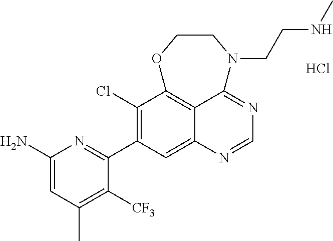 [SMILES: CNCCN1CCOc2c(Cl)c(-c3nc(N)cc(C)c3C(F)(F)F)cc3ncnc1c23 ; Cl]

To a solution of tert-butyl (2-(9-(6-amino-4-methyl-3-(trifluoromethyl)pyridin-2-yl)-8-chloro-5,6-dihydro-4H-[1,4]oxazepino[5,6,7-de]quinazolin-4-yl)ethyl)(methyl)carbamate (70.0 mg, 0.130 mmol) in methyl alcohol (5 mL) was added HCl/dioxane (5 mL, 4M). Stirred at room temperature for 1 h. The resulting mixture was concentrated under vacuum to afford 59.6 mg (crude) product. LC-MS: (ESI, m/z): [M+H]⁺=453.3. The crude was used for the next step without further purification.

Step 6: 6-(8-Chloro-4-(2-(methylamino)ethyl)-5,6-dihydro-4H-[1,4]oxazepino[5,6,7-de]quinazolin-9-yl)-4-methyl-5-(trifluoromethyl)pyridin-2-amine

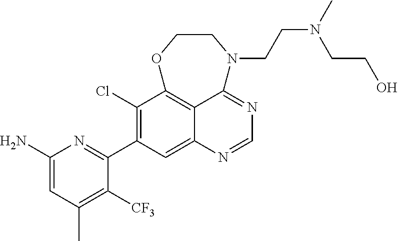 [SMILES: Cc1cc(N)nc(-c2cc3ncnc4c3c(c2Cl)OCCN4CCN(C)CCO)c1C(F)(F)F]

To a solution of 6-(8-chloro-4-(2-(methylamino)ethyl)-5,6-dihydro-4H-[1,4]oxazepino[5,6,7-de]quinazolin-9-yl)-4-methyl-5-(trifluoromethyl)pyridin-2-amine hydrochloride (57.3 mg, 0.130 mmol) in acetonitrile (5 mL) was added DIPEA (49.9 mg, 0.390 mmol). Then 2-bromoethan-1-ol (31.5 mg, 0.250 mmol) was added. The reaction mixture was stirred at 50° C. for 8 hours and concentrated under vacuum. The residue was purified by pre-packed C18 column (solvent gradient: 0%-100% ACN in water (0.1% NH₄HCO₃)) to yield 31.0 mg (not pure) of the title compound, which was re-purified by Prep-HPLC (Column: XBridge Prep OBD C18 Column, 30×150 mm 5 um; Mobile Phase A: Water (10 mmol/L NH₄HCO₃), Mobile Phase B: ACN; Flow rate: 60 mL/min; Gradient: 17 B to 47 B in 10 min; 254 nm; RT: 9.17.) to yield 14.8 mg (23% yield) of the title compound. LC-MS: (ESI, m/z): [M+H]⁺=497.2. ¹H NMR (400 MHz, DMSO-d6, ppm) δ 8.38 (s, 1H), 7.12 (s, 1H), 6.73 (s, 2H), 6.44 (s, 1H), 4.71-4.55 (m, 2H), 4.35 (t, J=5.2 Hz, 1H), 4.05-3.83 (m, 4H), 3.45 (dd, J=11.6, 6.0 Hz, 2H), 2.68 (t, J=6.0 Hz, 2H), 2.50-2.45 (m, 2H), 2.36 (s, 3H), 2.28 (s, 3H)

Compounds 501-506 were prepared following a similar experimental procedure (using appropriately substituted reagents) as described Example 501.

Example 607 and 508: 2-((5S)-9-(6-Amino-4-methyl-3-(trifluoromethyl)pyridin-2-yl)-8-chloro-4-(pyrrolidin-3-yl)-5,6-dihydro-4H-[1,4]oxazepino[5,6,7-de]quinazolin-5-yl)acetonitrile (two unknown single isomers)

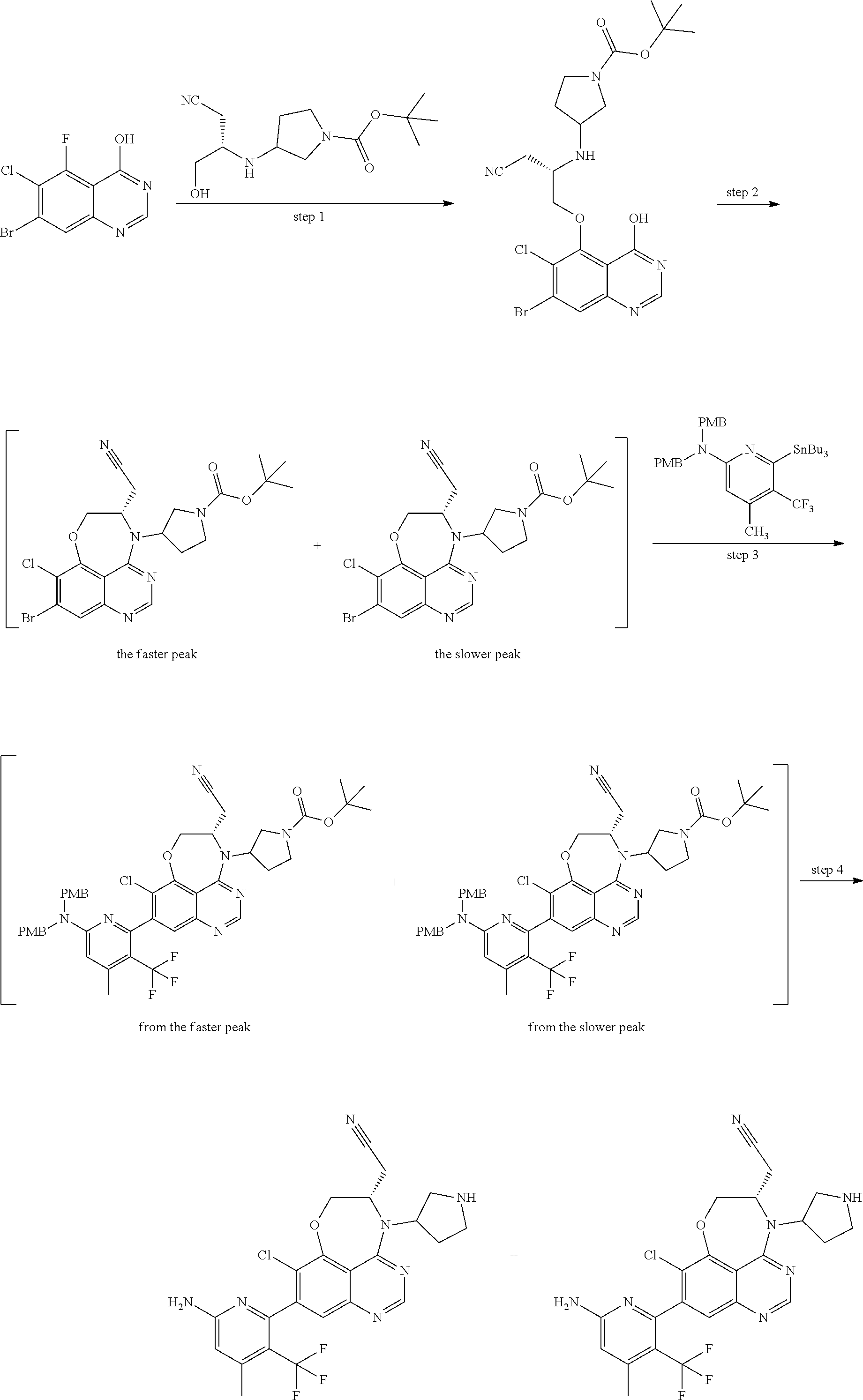

Step 1: tert-Butyl 3-(((S)-1-((7-bromo-6-chloro-4-hydroxyquinazolin-5-yl)oxy)-3-cyanopropan-2-yl)amino)pyrrolidine-1-carboxylate

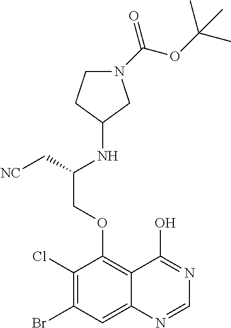 [SMILES: CC(C)(C)OC(=O)N1CCC(N[C@@H](CC#N)COc2c(Cl)c(Br)cc3ncnc(O)c23)C1]

Under nitrogen, to a solution of tert-butyl 3-[[(1S)-1-(cyanomethyl)-2-hydroxyethyl]amino]pyrrolidine-1-carboxylate (139 mg, 0.520 mmol, intermediate 15) in tetrahydrofuran (3 mL) was added 60% NaH (31.4 mg, 0.790 mmol) at 0° C. The resulting solution was stirred at room temperature for 0.5 h. Then 7-bromo-6-chloro-5-fluoro-quinazolin-4-ol (72.1 mg, 0.260 mmol) was added. Stirred at room temperature for 2 hours. The reaction was quenched with water and concentrated under vacuum. The residue was purified by flash chromatography on silica gel (gradient: 0%-10% MeOH/DCM) to yield 130 mg (95.3% yield) of the title compound as a yellow solid. LC-MS: (ESI, m/z): [M+H]⁺⁼526.1.

Step 2: tert-Butyl 3-((S)-9-bromo-8-chloro-5-(cyanomethyl)-5,6-dihydro-4H-[1,4]oxazepino[5,6,7-de]quinazolin-4-yl)pyrrolidine-1-carboxylate (Two Single Unknown Isomers)

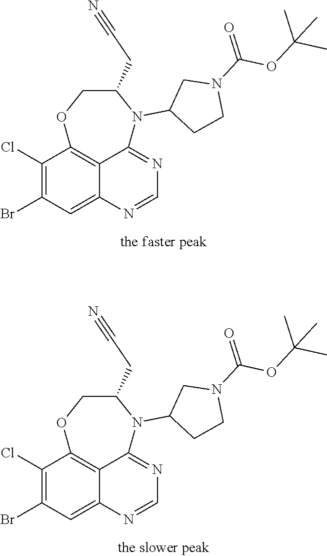 [SMILES: CC(C)(C)OC(=O)N1CCC(N2c3ncnc4cc(Br)c(Cl)c(c34)OC[C@@H]2CC#N)C1 ; CC(C)(C)OC(=O)N1CCC(N2c3ncnc4cc(Br)c(Cl)c(c34)OC[C@@H]2CC#N)C1]

To a solution of tert-butyl 3-[[(1S)-1-[(7-bromo-6-chloro-4-hydroxy-quinazolin-5-yl)oxymethyl]-2-cyano-ethyl]amino]pyrrolidine-1-carboxylate (110 mg, 0.210 mmol) and DIPEA (108 mg, 0.840 mmol) in 1,2-Dichloroethane (4 mL) was added BOPCl (159 mg, 0.630 mmol) at room temperature. The resulting solution was stirred at room temperature for 1 h. Then the resulting solution was stirred at 60° C. overnight and concentrated under vacuum. The residue was purified by pre-packed C18 column (solvent gradient: 0-100% ACN in water (0.1% FA)) to yield 65 mg (mixture of two isomers) of the title compound as a white solid. The mixture was separated by Chiral-HPLC (Column: CHIRALPAK IG, 2*25 cm, 5 μm; Mobile Phase A: Hex (0.5% 2M NH3-MeOH)-HPLC, Mobile Phase B: EtOH-HPLC; Flow rate: 18 mL/min; Gradient: 50% B to 50% B in 22 min; Wave Length: 220/254 nm; RT1(min): 11.31: RT2(min): 22.07; Sample Solvent: EtOH-HPLC; Injection Volume: 2 mL; Number Of Runs: 5) to yield 22.1 mg of the faster peak and 24.1 mg of the slower peak as light yellow solids. LC-MS: (ESI, m/z): [M+H]⁺=508.1.

Step 3: tert-Butyl 3-((S)-9-(6-(bis(4-methoxybenzyl)amino)-4-methyl-3-(trifluoromethyl)pyridin-2-yl)-8-chloro-5-(cyanomethyl)-5,6-dihydro-4H-[1,4]oxazepino[5,6,7-de]quinazolin-4-yl)pyrrolidine-1-carboxylate (two single unknown isomers)

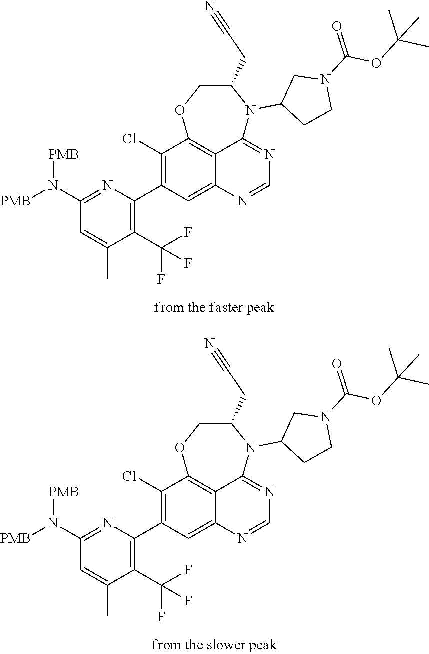

Under nitrogen, a mixture of tert-butyl 3-((S)-9-bromo-8-chloro-5-(cyanomethyl)-5,6-dihydro-4H-[1,4]oxazepino[5,6,7-de]quinazolin-4-yl)pyrrolidine-1-carboxylate (20.1 mg, 0.040 mmol, the faster peak of step 2), N,N-bis(4-methoxybenzyl)-4-methyl-6-(tributylstannyl)-5-(trifluoromethyl)pyridin-2-amine (55.6 mg, 0.080 mmol, intermediate 10), Pd(PPh₃)₄ (9.20 mg, 0.010 mmol), CuI (1.10 mg, 0.010 mmol) and LiCl (4.30 mg, 0.100 mmol) in 1,4-Dioxane (2 mL) was stirred at 110° C. for 1 h. The mixture was cooled to room temperature and the solid was filtered off. The filtrate was concentrated under reduced pressure. The residue was purified by flash chromatography on silica gel (gradient: 0%-40% EtOAc/DCM) to yield 27.1 mg (81.2% yield) of the title compound as. LC-MS: (ESI, m/z): [M+H]⁺=844.3.

Analogous to method described as above, the other isomer 22.1 mg was prepared from tert-butyl 3-((S)-9-bromo-8-chloro-5-(cyanomethyl)-5,6-dihydro-4H-[1,4]oxazepino[5,6,7-de]quinazolin-4-yl)pyrrolidine-1-carboxylate (the slower peak of step 2) and N,N-bis(4-methoxybenzyl)-4-methyl-6-(tributylstannyl)-5-(trifluoromethyl)pyridin-2-amine (intermediate 10).

Step 4: 2-((5S)-9-(6-Amino-4-methyl-3-(trifluoromethyl)pyridin-2-yl)-8-chloro-4-(pyrrolidin-3-yl)-5,6-dihydro-4H-[1,4]oxazepino[5,6,7-de]quinazolin-5-yl)acetonitrile (two single unknown isomers)

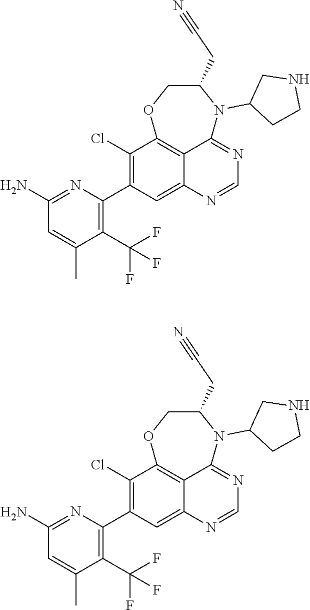 [SMILES: Cc1cc(N)nc(-c2cc3ncnc4c3c(c2Cl)OC[C@H](CC#N)N4C2CCNC2)c1C(F)(F)F ; Cc1cc(N)nc(-c2cc3ncnc4c3c(c2Cl)OC[C@H](CC#N)N4C2CCNC2)c1C(F)(F)F]

A solution of tert-butyl 3-((S)-9-(6-(bis(4-methoxybenzyl)amino)-4-methyl-3-(trifluoromethyl)pyridin-2-yl)-8-chloro-5-(cyanomethyl)-5,6-dihydro-4H-[1,4]oxazepino[5,6,7-de]quinazolin-4-yl)pyrrolidine-1-carboxylate (26.9 mg, 0.030 mmol, the faster peak of step 3) in 2,2,2-trifluoroacetic acid (1 mL) was stirred at 50° C. for 5 hours. Concentrated under vacuum. The residue was purified by Prep-HPLC (Column: XBridge Prep C18 OBD Column, 30*100 mm, 5 μm; Mobile Phase A: Water (10 mmol/L NH4HCO3), Mobile Phase B: ACN: Flow rate: 60 mL/min; Gradient: 16% B to 40% B in 9 min, 40% B; Wave Length: 254/220 nm; RT1(min): 8.85; Number Of Runs: 0) to yield 5.90 mg (36.7% yield) of the title compound. LC-MS: (ESI, m/z): [M+H]⁺=504.1. ¹H NMR (400 MHz, DMSO-d₆, ppm) δ 8.49 (s, 1H), 7.21 (s, 1H), 6.76 (d, J=12.1 Hz, 2H), 6.45 (s, 1H), 5.29-5.15 (m, 1H), 4.85-4.78 (m, 1H), 4.67-4.50 (m, 2H), 3.77-3.55 (m, 1H), 3.17-3.08 (m, 1H), 3.08-2.98 (m, 1H), 2.98-2.82 (m, 3H), 2.81-2.70 (m, 1H), 2.38 (s, 3H), 2.25-1.96 (m, 1H)

A solution of tert-butyl 3-((S)-9-(6-(bis(4-methoxybenzyl)amino)-4-methyl-3-(trifluoromethyl)pyridin-2-yl)-8-chloro-5-(cyanomethyl)-5,6-dihydro-4H-[1,4]oxazepino[5,6,7-de]quinazolin-4-yl)pyrrolidine-1-carboxylate (21.9 mg, 0.030 mmol, the slower peak of step 3) in 2,2,2-trifluoroacetic acid (1 mL) was stirred at 50° C. for 5 hours. Concentrated under vacuum. The residue was purified by Prep-HPLC (Column: XBridge Prep OBD C18 Column, 30*150 mm, 5 μm; Mobile Phase A: Water (10 mmol/L NH4HCO3), Mobile Phase B: ACN; Flow rate: 60 mL/min; Gradient: 22% B to 45% B in 9 min, 45% B: Wave Length: 220/254 nm; RT1(min): 9.92; Number Of Runs: 0) to yield 4.40 mg (33.7% yield) of the title compound. LC-MS: (ESI, m/z): [M+H]⁺=504.1. ¹H NMR (400 MHz, DMSO-d₆, ppm) δ 8.50 (s, 1H), 7.21 (s, 1H), 6.79 (d, J=12.4 Hz, 2H), 6.46 (s, 1H), 5.25-5.08 (m, 1H), 4.88-4.72 (m, 1H), 4.70-4.46 (m, 2H), 3.62-3.45 (m, 1H), 3.19-3.09 (m, 2H), 3.09-2.92 (m, 3H), 2.89-2.77 (m, 1H), 2.35 (s, 3H), 2.25-2.07 (m, 1H), 1.92-1.76 (m, 1H).

Example 509: 6-(8-Chloro-4-(2-(dimethylamino)ethyl)-2-(((2S,4R)-4-fluoro-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

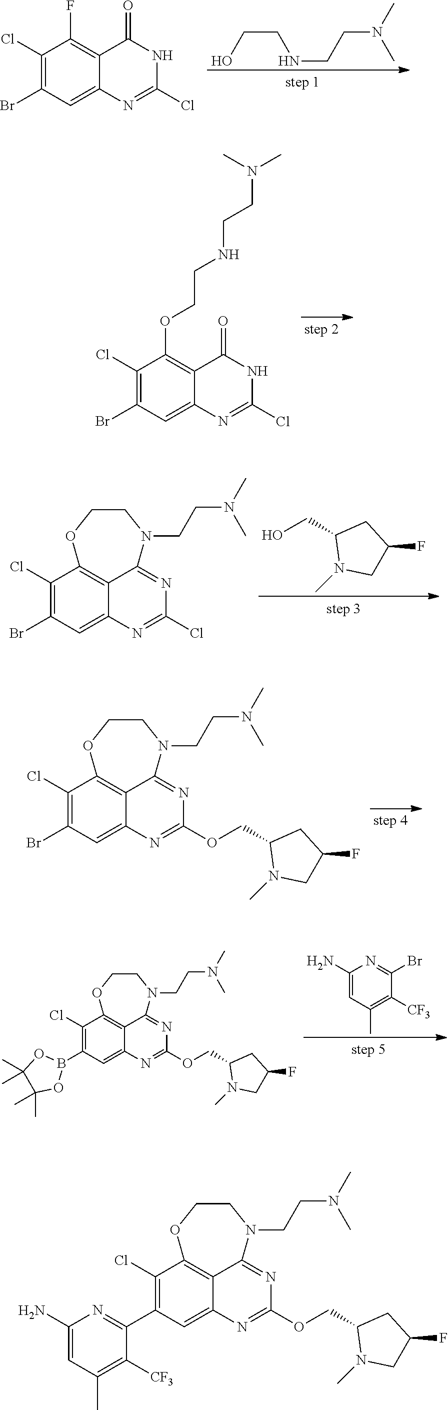

Step 1: 7-Bromo-2,6-dichloro-5-(2-((2-(dimethylamino)ethyl)amino)ethoxy) quinazolin-4(3H)-one

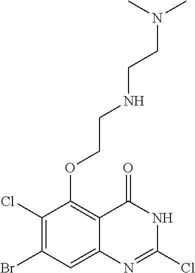 [SMILES: CN(C)CCNCCOc1c(Cl)c(Br)cc2nc(Cl)[nH]c(=O)c12]

Under nitrogen, to a solution of 2-((2-(dimethylamino)ethyl)amino)ethan-1-ol (235 mg, 1.78 mmol) in tetrahydrofuran (16 mL) was added NaH (214 mg, 5.35 mmol) at 0° C. The solution was stirred at room temperature for 30 min. Then 7-bromo-2,6-dichloro-5-fluoroquinazolin-4(3H)-one (552 mg, 1.77 mmol) was added at 0° C. The reaction mixture was stirred at 65° C. for 1 hours. The reaction was quenched with water at 0° C., extracted with ethyl acetate. The combined organic layers were dried over anhydrous sodium sulfate and concentrated under vacuum. The residue was purified by flash chromatography on silica gel (gradient: 0%-20% methanol/dichloromethane) to yield 230 mg (30% yield) of the title compound as a yellow solid. LC-MS: (ESI, m/z): [M+H]⁺=423.

Step 2: 2-(9-Bromo-2,8-dichloro-5,6-dihydro-4H-[1,4]oxazepino[5,6,7-de]quinazolin-4-yl)-N,N-dimethylethan-1-amine

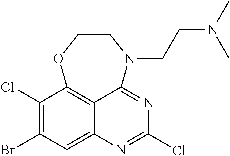 [SMILES: CN(C)CCN1CCOc2c(Cl)c(Br)cc3nc(Cl)nc1c23]

Under nitrogen, a solution of 7-bromo-2,6-dichloro-5-(2-((2-(dimethylamino)ethyl)amino)ethoxy)quinazolin-4(3H)-one (350 mg, 0.830 mmol), PyAOP (861 mg, 1.65 mmol) and DBU (376 mg, 2.48 mmol) in dichloromethane (20 mL) was stirred at room temperature for 3 hours. The reaction mixture was diluted with water and extracted with dichloromethane. The organic layer was dried over anhydrous sodium sulfate and concentrated under vacuum. The residue was purified by flash chromatography on silica gel (gradient: 0%-20% methanol/dichloromethane) to yield 280 mg (84% yield) of the title compound as a yellow solid. LC-MS: (ESI, m/z): [M+H]⁺=405.

Step 3: 2-(9-Bromo-8-chloro-2-(((2S,4R)-4-fluoro-1-methylpyrrolidin-2-yl)methoxy)-5,6-dihydro-4H-[1,4]oxazepino[5,6,7-de]quinazolin-4-yl)-N,N-dimethylethan-1-amine

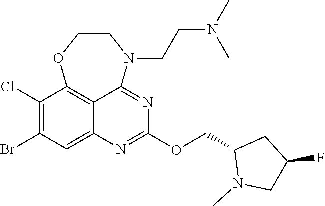 [SMILES: CN(C)CCN1CCOc2c(Cl)c(Br)cc3nc(OC[C@@H]4C[C@@H](F)CN4C)nc1c23]

Under nitrogen, to a solution of ((2S,4R)-4-fluoro-1-methylpyrrolidin-2-yl) methanol (49.5 mg, 0.370 mmol, intermediate 8) in tetrahydrofuran (10 mL) was added 60% NaH (20.0 mg, 0.500 mmol) at 0° C. The solution was stirred at room temperature for min. Then 2-(9-bromo-2,8-dichloro-5,6-dihydro-4H-[1,4]oxazepino[5,6,7-de]quinazolin-4-yl)-N,N-dimethylethan-1-amine (100 mg, 0.250 mmol) was added at 0° C.

The reaction mixture was stirred at 50° C. for 2 hours. The reaction was quenched by acetic acid and diluted with water. The resulting solution was extracted with ethyl acetate. The combined organic layers were dried over anhydrous sodium sulfate and concentrated under vacuum. The residue was purified by flash chromatography on silica gel (gradient: 0%-20% methanol/dichloromethane) to yield 120 mg (96% yield) of the title compound as a yellow solid. LC-MS: (ESI, m/z): [M+H]⁺=502.

Step 4: 2-(8-Chloro-2-(((2S,4R)-4-fluoro-1-methylpyrrolidin-2-yl)methoxy)-9-(4,4,5,5-tetramethyl-1,3,2-dioxaborolan-2-yl)-5,6-dihydro-4H-[1,4]oxazepino[5,6,7-de]quinazolin-4-yl)-N,N-dimethylethan-1-amine

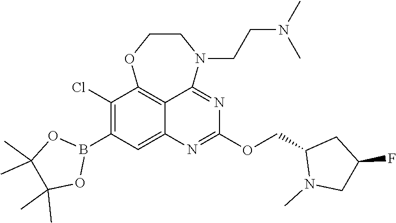 [SMILES: CN(C)CCN1CCOc2c(Cl)c(B3OC(C)(C)C(C)(C)O3)cc3nc(OC[C@@H]4C[C@@H](F)CN4C)nc1c23]

Under nitrogen, a mixture of 2-(9-bromo-8-chloro-2-(((2S,4R)-4-fluoro-1-methylpyrrolidin-2-yl)methoxy)-5,6-dihydro-4H-[1,4]oxazepino[5,6,7-de]quinazolin-4-yl)-N,N-dimethylethan-1-amine (100 mg, 0.200 mmol), 4,4,4′,4′,5,5,5′,5′-octamethyl-2,2′-bi(1,3,2-dioxaborolane) (152 mg, 0.600 mmol), Pd(dppf)Cl₂ (29.0 mg, 0.0400 mmol) and KOAc (59.0 mg, 0.600 mmol) in 1,4-dioxane (14 mL) was stirred at 80° C. for 3 h. The reaction mixture was diluted with water and extracted with ethyl acetate. The organic layer was dried over anhydrous sodium sulfate and concentrated under vacuum. A mixture of petroleum ether/ethyl acetate (9:1) was added. The solids were filtered off and the filtrate was concentrated under reduced pressure to yield 189 mg (crude) of the title compound as a yellow solid. LC-MS: (ESI, m/z): [M+H]⁺=468 (corresponding boronic acid mass signal). The crude was used for next step without further purification.

Step 5: 6-(8-chloro-4-(2-(dimethylamino)ethyl)-2-(((2S,4R)-4-fluoro-1-methylpyrrolidin-2-yl)methoxy)-5,6-dihydro-4H-[1,4]oxazepino[5,6,7-de]quinazolin-9-yl)-4-methyl-5-(trifluoromethyl)pyridin-2-amine

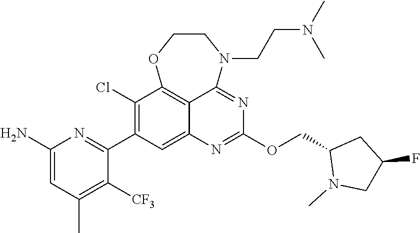 [SMILES: Cc1cc(N)nc(-c2cc3nc(OC[C@@H]4C[C@@H](F)CN4C)nc4c3c(c2Cl)OCCN4CCN(C)C)c1C(F)(F)F]

Under nitrogen, a solution of 6-bromo-4-methyl-5-(trifluoromethyl)pyridin-2-amine (4.80 mg, 0.0200 mmol) and 2-(8-Chloro-2-(((2S,4R)-4-fluoro-1-methylpyrrolidin-2-yl)methoxy)-9-(4,4,5,5-tetramethyl-1,3,2-dioxaborolan-2-yl)-5,6-dihydro-4H-[1,4]oxazepino[5,6,7-de]quinazolin-4-yl)-N,N-dimethylethan-1-amine (94.0 mg, 0.180 mmol), Pd(PPh₃)₄(3.20 mg, 0.00300 mmol) and K₂CO₃ (7.50 mg, 0.0500 mmol) in 1,2-dimethoxyethane (1 mL) and water (0.2 mL) was stirred at 100° C. for 1 h. The reaction mixture was partitioned between water and EtOAc. The organic phase was concentrated under reduced pressure. The residue was purified by pre-packed C18 column (solvent gradient: 0-100% ACN in water (0.05% NH₄HCO₃)) to yield 20 mg crude product as a yellow solid. The crude product was repurified by Prep-HPLC (Column: XBridge Prep OBD C18 Column, 30×150 mm Sum; Mobile Phase A: Water (10 mmol/L NH₄HCO₃), Mobile Phase B: ACN; Flow rate: 60 mL/min; Gradient: 38% B to 68% B in 7 min; 254 nm; R_{T1}: 6.07 min;) to yield 7.1 mg (59% yield) of title compound. LC-MS: (ESI, m/z): [M+H]⁺=598. ¹H NMR (400 MHz, DMSO-d6, ppm) δ 6.91 (s, 1H), 6.72 (s, 2H), 6.43 (s, 1H), 5.18 (d, J=55.8 Hz, 1H), 4.65-4.49 (m, 2H), 4.40-4.28 (m, 1H), 4.27-4.15 (m, 1H), 4.05-3.90 (m, 3H), 3.91-3.79 (m, 1H), 3.50-3.35 (m, 1H), 2.94-2.85 (m, 1H), 2.55 (t, J=6.3 Hz, 2H), 2.39 (s, 4H), 2.34 (s, 3H), 2.21 (s, 6H), 2.16-2.04 (m, 1H), 1.96-1.75 (m, 1H).

Compounds 509-512 were was prepared following a similar experimental procedure (using appropriately substituted reagents) as described Example 509:

Example 513 and 514: 6-((R)-8-Chloro-4-(2-(dimethylamino)ethyl)-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (Two Atropisomes)

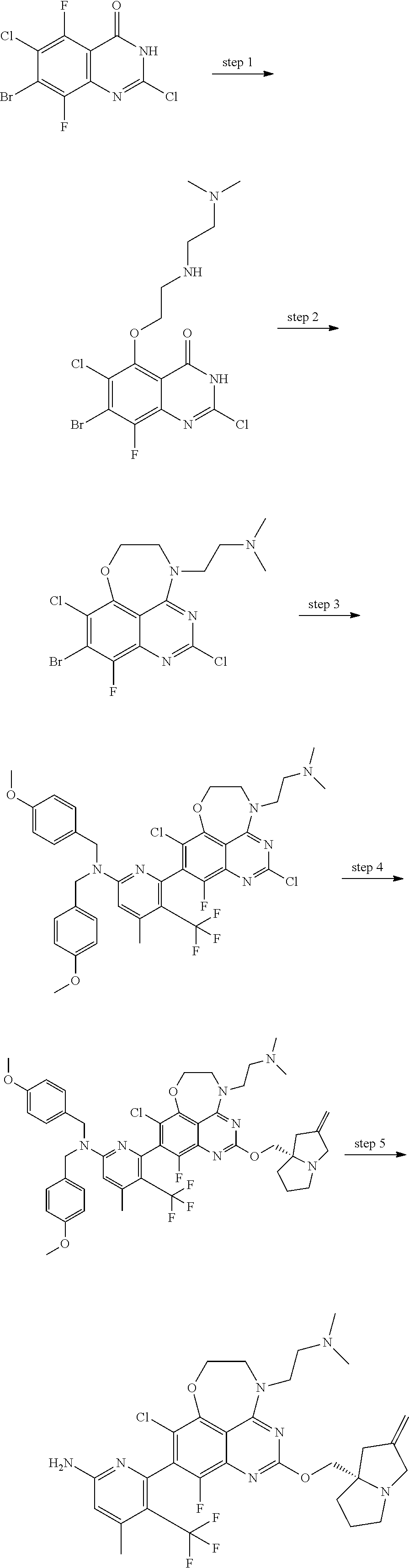 [SMILES: O=c1[nH]c(Cl)nc2c(F)c(Br)c(Cl)c(F)c12 ; CN(C)CCNCCOc1c(Cl)c(Br)c(F)c2nc(Cl)[nH]c(=O)c12 ; CN(C)CCN1CCOc2c(Cl)c(Br)c(F)c3nc(Cl)nc1c23 ; COc1ccc(CN(Cc2ccc(OC)cc2)c2cc(C)c(C(F)(F)F)c(-c3c(Cl)c4c5c(nc(Cl)nc5c3F)N(CCN(C)C)CCO4)n2)cc1 ; C=C1CN2CCC[C@@]2(COc2nc3c4c(c(Cl)c(-c5nc(N(Cc6ccc(OC)cc6)Cc6ccc(OC)cc6)cc(C)c5C(F)(F)F)c(F)c4n2)OCCN3CCN(C)C)C1 ; C=C1CN2CCC[C@@]2(COc2nc3c4c(c(Cl)c(-c5nc(N)cc(C)c5C(F)(F)F)c(F)c4n2)OCCN3CCN(C)C)C1]

Step 1: 7-Bromo-2,6-dichloro-5-(2-((2-(dimethylamino)ethyl)amino)ethoxy)-8-fluoroquinazolin-4(3H)-one

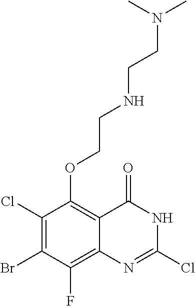 [SMILES: CN(C)CCNCCOc1c(Cl)c(Br)c(F)c2nc(Cl)[nH]c(=O)c12]

Under nitrogen, to a solution of 7-bromo-2,6-dichloro-5,8-difluoroquinazolin-4(3H)-one (190 mg, 1.44 mmol) in tetrahydrofuran (6 mL) was added 60% NaH (190 mg, 4.77 mmol) and the solution was stirred at room temperature for 10 min. Then 7-bromo-2,6-dichloro-5,8-difluoro-3H-quinazolin-4-one (524 mg, 1.59 mmol, intermediate 1) was added. The resulting solution was stirred at room temperature for 1 hour. The reaction was quenched with AcOH, partitioned between water and DCM. The combined organic layers were dried over anhydrous Na₂SO₄ and concentrated under vacuum. The residue was purified by pre-packed C18 column (solvent gradient: 0-100% ACN in water (0.05% NH₄HCO₃)) to yield 510 mg (72% yield) of the title compound as a brown solid. LC-MS: (ESI, m/z): [M+H]⁺=441.

Step 2: 2-(9-Bromo-2,8-dichloro-10-fluoro-5,6-dihydro-4H-[1,4]oxazepino[5,6,7-de]quinazolin-4-yl)-N,N-dimethylethan-1-amine

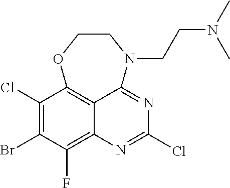 [SMILES: CN(C)CCN1CCOc2c(Cl)c(Br)c(F)c3nc(Cl)nc1c23]

A solution of 7-bromo-2,6-dichloro-5-(2-((2-(dimethylamino)ethyl)amino)ethoxy)-8-fluoroquinazolin-4(3H)-one (984 mg, 2.23 mmol), DIPEA (4.32 g, 33.3 mmol) and BOPCl (1.70 g, 6.67 mmol) in dichloromethane (15 mL) was stirred at room temperature for 12 hours. Concentrated under vacuum. The residue was purified by pre-packed C18 column (solvent gradient: 0-100% ACN in water (0.05% NH₄HCO₃)) to yield 338 mg (35% yield) of the title compound as a yellow solid. LC-MS: (ESI, m/z): [M+H]⁺=423.

Step 3: 6-(2,8-Dichloro-4-(2-(dimethylamino)ethyl)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

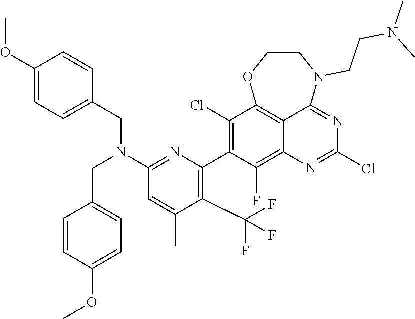 [SMILES: COc1ccc(CN(Cc2ccc(OC)cc2)c2cc(C)c(C(F)(F)F)c(-c3c(Cl)c4c5c(nc(Cl)nc5c3F)N(CCN(C)C)CCO4)n2)cc1]

Under nitrogen, to a solution of 2-(9-bromo-2,8-dichloro-10-fluoro-5,6-dihydro-4H-[1,4]oxazepino[5,6,7-de]quinazolin-4-yl)-N,N-dimethylethan-1-amine (200 mg, 0.473 mmol) in tetrahydrofuran (1.5 mL) was added iPrMgCl·LiCl (0.40 mL, 1.3M in THF) at −78° C. and the reaction was stirred at −78° C. for 1 hour. Then ZnCl₂ (0.28 mL, 2M in THF) at −78° C. was added. The solution was stirred at −78° C. for 15 min and additional 15 min at room temperature. Then preformed solution of 6-bromo-N,N-bis(4-methoxybenzyl)-4-methyl-5-(trifluoromethyl)pyridin-2-amine (197 mg, 0.399 mmol, intermediate 10, step 1) and PdCl₂(PPh₃)₂(29.0 mg, 0.0413 mmol) in tetrahydrofuran (1.5 mL) was added. Stirred at 50° C. for 12 hours. The mixture was partitioned between water and EtOAc. The organic lay was concentrated under vacuum. The residue was purified by pre-packed C18 column (solvent gradient: 0-100% ACN in water (0.05% NH₄HCO₃)) to yield 107 mg (29% yield) of the title compound as a light yellow solid. LC-MS: (ESI, m/z): [M+H]⁺=759.

Step 4: 6-(8-Chloro-4-(2-(dimethylamino)ethyl)-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

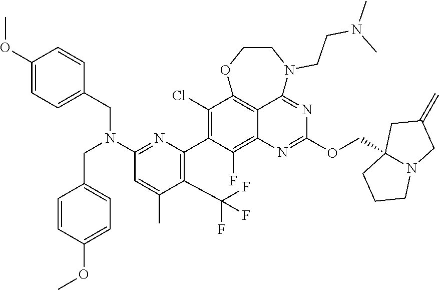 [SMILES: C=C1CN2CCC[C@@]2(COc2nc3c4c(c(Cl)c(-c5nc(N(Cc6ccc(OC)cc6)Cc6ccc(OC)cc6)cc(C)c5C(F)(F)F)c(F)c4n2)OCCN3CCN(C)C)C1]

Under nitrogen, to a solution of (S)-(2-methylenetetrahydro-1H-pyrrolizin-7a(5H)-yl)methanol (25.9 mg, 0.169 mmol, intermediate 5) in tetrahydrofuran (2 mL) was added 60% NaH (20.3 mg, 0.510 mmol) at 0° C. The reaction was stirred at room temperature for 15 mins. Then 6-(2,8-dichloro-4-(2-(dimethylamino)ethyl)-10-fluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (107 mg, 0.140 mmol) was added and the reaction was stirred at room temperature for 12 hours. The reaction was quenched the with AcOH, diluted with water and extract three times with DCM. The combined organic layers were dried over anhydrous Na₂SO₄ and evaporated under vacuum. The residue was purified by flash chromatography on silica gel (0-20% MeOH in DCM) to yield 75 mg (60% yield) of the title compound as a yellow solid. LC-MS: (ESI, m/z): [M+H]⁺=876.

Step 5: 6-((R)-8-Chloro-4-(2-(dimethylamino)ethyl)-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine

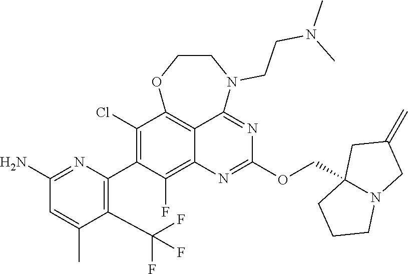 [SMILES: C=C1CN2CCC[C@@]2(COc2nc3c4c(c(Cl)c(-c5nc(N)cc(C)c5C(F)(F)F)c(F)c4n2)OCCN3CCN(C)C)C1]

A solution of 6-(8-chloro-4-(2-(dimethylamino)ethyl)-10-fluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (75.0 mg, 0.0857 mmol) in TFA (3 mL) was stirred at 50° C. for 5 hours. Concentrated under vacuum. The residue was purified by pre-packed C18 column (solvent gradient: 0-100% ACN in water (0.05% NH₄HCO₃)) and then further purified by Prep-HPLC (Column: XBridge Prep OBD C18 Column, 30×150 mm Sum; Mobile Phase A: Water (10 mmol/L NH₄HCOs), Mobile Phase B: ACN; Flow rate: 60 mL/min; Gradient: 32% B to 62% B in 10 min; 254 nm; R_{T}:8.90 min) to yield 17 mg mixture of two atropisomers. The atropisomers were separated by Prep Chiral HPLC (Column: CHIRAL ART Cellulose-SC, 2×25 cm, 5 um; Mobile Phase A: Hex (10 mmol/L NH₃), Mobile Phase B: EtOH; Flow rate: 18 mL/min; Gradient: 50% B to 50% B in 14 min; R_{T1}:7.007 min; RT2:9.553 min) to yield 5.0 mg Compound 513 (faster peak) and 3.9 mg Compound 514 (slower peak).

Compound 513: LC-MS: (ESI, m/z): [M+H]⁺=636. ¹H NMR (300 MHz, DMSO-d6, ppm) δ 6.78 (s, 2H), 6.47 (s, 1H), 4.90 (s, 2H), 4.65-4.45 (m, 2H), 4.12-3.80 (m, 6H), 3.65-3.55 (m, 2H), 3.08-2.98 (m, 1H), 2.70-2.55 (m, 5H), 2.30 (s, 3H), 2.25 (s, 6H), 2.01-1.73 (m, 4H)

Compound 514: LC-MS: (ESI, m/z): [M+H]⁺=636. ¹H NMR (400 MHz, DMSO-d6, ppm) δ 6.77 (s, 2H), 6.46 (s, 1H), 4.87 (s, 2H), 4.60-4.45 (m, 2H), 4.09-3.86 (m, 5H), 3.85-3.72 (m, 1H), 3.53 (d, J=14.1 Hz, 1H), 3.18 (d, J=14.1 Hz, 1H), 3.02-2.94 (m, 1H), 2.59-2.51 (m, 4H), 2.35 (s, 4H), 2.21 (s, 6H), 1.99-1.90 (m, 1H), 1.90-1.70 (m, 2H), 1.70-1.60 (m, 1H)

Compounds 515-522 were prepared following a similar experimental procedure (using appropriately substituted reagents) as described Example 513 and 514.

Example 523 and 524: 6-((R)-4-(2-(Dimethylamino)ethyl)-8,10-difluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (two single unknown atropisomers)

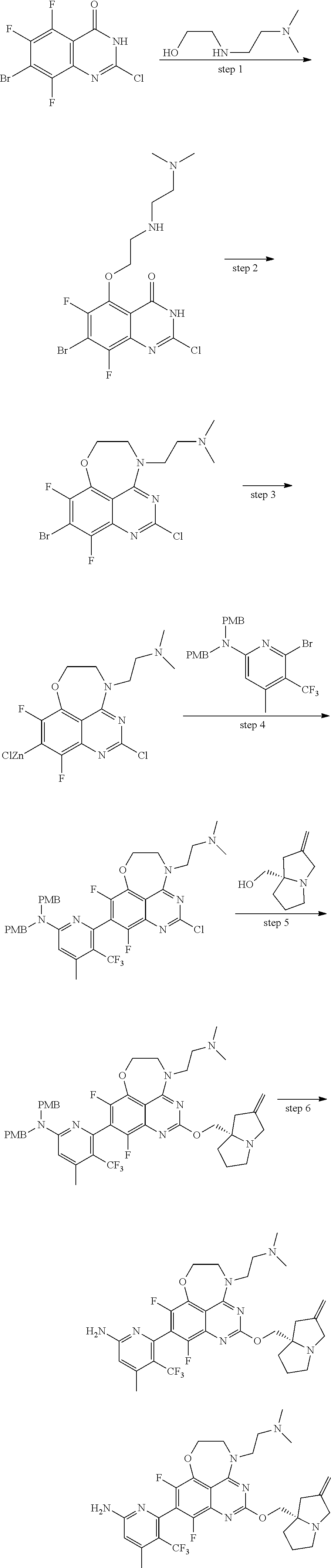

Step 1: 7-Bromo-2-chloro-5-(2-((2-(dimethylamino)ethyl)amino)ethoxy)-6,8-difluoroquinazolin-4(3H-)-one

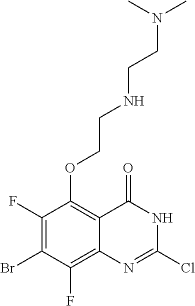 [SMILES: CN(C)CCNCCOc1c(F)c(Br)c(F)c2nc(Cl)[nH]c(=O)c12]

Under nitrogen, to a solution of 2-((2-(dimethylamino)ethyl)amino)ethan-1-ol (338 mg, 2.56 mmol) in tetrahydrofuran (10 mL) was added 60% NaH (384 mg, 9.62 mmol) at 0° C. The reaction was stirred at room temperature for 0.5 h. Then 7-bromo-2-chloro-5,6,8-trifluoroquinazolin-4(3H)-one (1.00 g, 3.19 mmol, intermediate 3) was added. Stirred at room temperature for 2 h. The reaction was diluted with water, extracted with ethyl acetate. The combined organic layers were dried over anhydrous sodium sulfate and concentrated under vacuum. The residue was purified by pre-packed C18 column (solvent gradient: 0-100% ACN in water (0.1% NH₄HCO₃)) to yield 760 mg (58% yield) of the title compound as a yellow solid. LC-MS: (ESI, m/z): [M+H]⁺=425

Step 2: 2-(9-Bromo-2-chloro-8,10-difluoro-5,6-dihydro-4H-[1,4]oxazepino[5,6,7-de]quinazolin-4-yl)-N,N-dimethylethan-1-amine

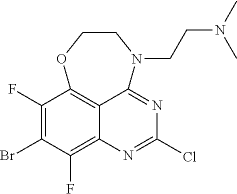 [SMILES: CN(C)CCN1CCOc2c(F)c(Br)c(F)c3nc(Cl)nc1c23]

A solution of 7-bromo-2-chloro-5-(2-((2-(dimethylamino)ethyl)amino)ethoxy)-6,8-difluoroquinazolin-4(3H)-one (760 mg, 1.79 mmol), BOPCl (1.37 g, 5.37 mmol) and DIPEA (3.47 g, 26.9 mmol) in dichloromethane (8 mL) was stirred at room temperature for 3 hours. Concentrated under vacuum. The residue was purified by pre-packed C18 column (solvent gradient: 0-100% ACN in water (0.1% NH₄HCOs)) to yield 177 mg (24% yield) of the title compound as a yellow solid. LC-MS: (ESI, m/z): [M+H]⁺=407.

Step 3: (2-Chloro-4-(2-(dimethylamino)ethyl)-8,10-difluoro-5,6-dihydro-4H-[1,4]oxazepino[5,6,7-de]quinazolin-9-yl)zinc(II) chloride

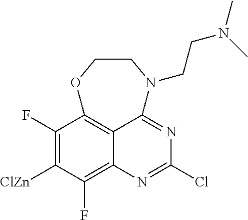

Under nitrogen, to a pre-cooling (−78° C.) solution of 2-(9-bromo-2-chloro-8,10-difluoro-5,6-dihydro-4H-[1,4]oxazepino[5,6,7-de]quinazolin-4-yl)-N,N-dimethylethan-1-amine (200 mg, 0.480 mmol) in tetrahydrofuran (1.2 mL) was added i-PrMg LiCl (0.4 mL, 1.3M in THF). Stirred at −78° C. for 1 h. Then ZnCl₂ in THE (0.240 mL, 2M in THF) was added and the mixture was stirred at room temperature for 1 h. The solution was used in the next step directly.

Step 4: 6-(2-Chloro-4-(2-(dimethylamino)ethyl)-8,10-difluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

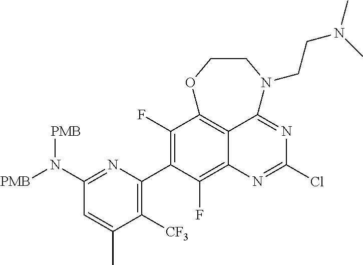

Under nitrogen, a solution of (2-chloro-4-(2-(dimethylamino)ethyl)-8,10-difluoro-5,6-dihydro-4H-[1,4]oxazepino[5,6,7-de]quinazolin-9-yl)zinc(II) chloride (crude from last step), 6-bromo-N,N-bis(4-methoxybenzyl)-4-methyl-5-(trifluoromethyl)pyridin-2-amine (243 mg, 0.490 mmol, intermediate 10, step 1) and PdCl₂(PPh₃)₂(48.8 mg, 0.0800 mmol) in THE was stirred at 50° C. overnight. The reaction was cooled to room temperature and partitioned between water and ethyl acetate. The combined organic layers were washed with brine, dried over anhydrous sodium sulfate and concentrated under vacuum. The residue was purified by flash chromatography on silica gel (gradient: 0%-20% MeOH/DCM) to yield 45 mg (12% yield) of the title compound as a yellow solid. LC-MS: (ESI, m/z): [M+H]⁺=743.

Step 5: 6-(4-(2-(Dimethylamino)ethyl)-8,10-difluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine

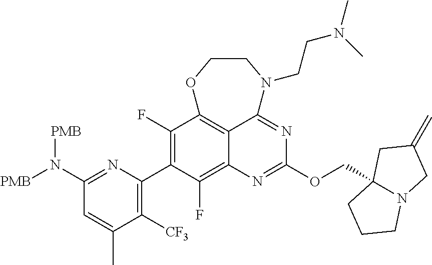

Under nitrogen, to a solution of (S)-(2-methylenetetrahydro-1H-pyrrolizin-7a(5H)-yl)methanol (10.8 mg, 0.070 mmol, intermediate 5) in THE (2 mL) was added 60% NaH (7.1 mg, 0.180 mmol) at 0° C. The solution was stirred at room temperature for 0.5 h. Then 6-(2-chloro-4-(2-(dimethylamino)ethyl)-8,10-difluoro-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (44.0 mg, 0.0600 mmol) was added. Stirred at 40° C. overnight. The reaction was diluted with water, extracted with ethyl acetate. The combined organic layers were dried over anhydrous sodium sulfate and concentrated under vacuum. The residue was purified by pre-packed C18 column (solvent gradient: 0-100% ACN in water (0.1% NH₄HCO₃)) to yield 41 mg (70% purity) as a yellow solid. LC-MS: (ESI, m/z): [M+H]⁺=861.

Step 6: 6-(4-(2-(Dimethylamino)ethyl)-8,10-difluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-4-methyl-5-(trifluoromethyl)pyridin-2-amine (two single unknown atropisomers)

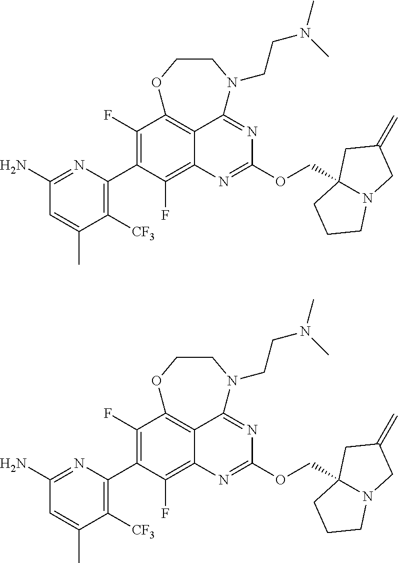 [SMILES: C=C1CN2CCC[C@@]2(COc2nc3c4c(c(F)c(-c5nc(N)cc(C)c5C(F)(F)F)c(F)c4n2)OCCN3CCN(C)C)C1 ; C=C1CN2CCC[C@@]2(COc2nc3c4c(c(F)c(-c5nc(N)cc(C)c5C(F)(F)F)c(F)c4n2)OCCN3CCN(C)C)C1]

A solution of 6-(4-(2-(dimethylamino)ethyl)-8,10-difluoro-2-(((S)-2-methylenetetrahydro-1H-pyrrolizin-7a(5H)-yl)methoxy)-5,6-dihydro-4H-[1,4]oxazepino[5,6,7-de]quinazolin-9-yl)-N,N-bis(4-methoxybenzyl)-4-methyl-5-(trifluoromethyl)pyridin-2-amine (41.0 mg, 0.050 mmol) in 2,2,2-trifluoroacetic acid (1.5 mL) was stirred at 50° C. for 3 hours. Concentrated under vacuum. The crude product was purified by Prep-HPLC with the following conditions: Column: Xselect CSH OBD Column 30*150 mm Sum; Mobile Phase A: Water (0.1% FA), Mobile Phase B: ACN; Flow rate: 60 mL/min; Gradient: 6% B to 25% B in 8 min; 220 nm; R_{T1}:7.82 min to yield 11 mg (mixture of two atropisomers) of the title compound. The atropisomers were separated by Chiral-Prep-HPLC with the following conditions: Column: (CHIRAL ART Cellulose-SC, 2*25 cm, 5 um; Mobile Phase A: Hex:DCM=3:1(0.5% 2M NH₃-MeOH), Mobile Phase B: EtOH; Flow rate: 20 mL/min; Gradient: 30% B to 30% B in 10 min; 220/254 nm; R_{T1}: 4.5 min; RT2:8.215 min;) to yield 3.1 mg (10.5% yield) Compound 523 and 2.5 mg (8.10% yield) of Example 524.

Compound 523: LC-MS: (ESI, m/z): [M+H]⁺=620. ¹H NMR (300 MHz, DMSO-do, ppm): δ 6.83 (s, 2H), 6.49 (s, 1H), 4.91 (s, 2H), 4.57-4.48 (m, 2H), 4.07-3.86 (m, 6H), 3.57 (s, 1H), 3.25 (s, 1H), 3.15 (s, 1H), 2.70-2.55 (m, 4H), 2.35 (s, 4H), 2.27 (s, 6H), 1.97-1.92 (m, 1H), 1.90-1.75 (m, 2H), 1.74-1.66 (m, 1H).

Compound 524: LC-MS: (ESI, m/z): [M+H]⁺=620. ¹H NMR (300 MHz, DMSO-de, ppm): δ 6.83 (s, 2H), 6.49 (s, 1H), 4.90 (s, 2H), 4.62-4.40 (m, 2H), 4.08-3.76 (m, 6H), 3.57 (d, J=14.1 Hz, 1H), 3.21 (d, J=14.1 Hz, 1H), 3.01 (dt, J=10.1, 5.4 Hz, 1H), 2.64-2.54 (m, 4H), 2.35 (s, 4H), 2.24 (s, 6H), 2.00-1.90 (m, 1H), 1.90-1.74 (m, 2H), 1.73-1.65 (m, 1H).

Example 525: 2-(9-(6-Amino-4-methyl-3-(trifluoromethyl)pyridin-2-yl)-8-chloro-10-fluoro-4-methyl-5,6-dihydro-4H-[1,4]oxazepino[5,6,7-de]quinazolin-5-yl)acetonitrile (four single unknown isomers)

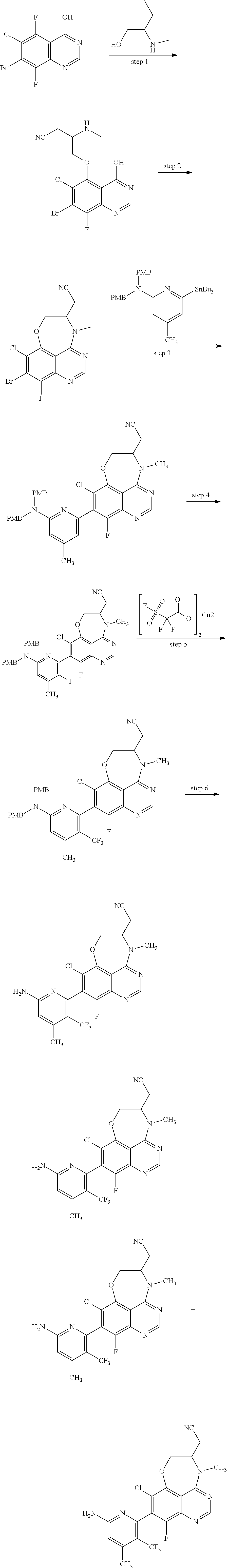

Step 1: 4-((7-Bromo-6-chloro-8-fluoro-4-hydroxyquinazolin-5-yl)oxy)-3-(methylamino)butanenitrile

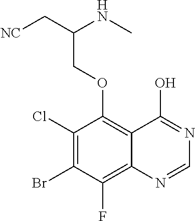 [SMILES: CNC(CC#N)COc1c(Cl)c(Br)c(F)c2ncnc(O)c12]

Under nitrogen, to a solution of 4-hydroxy-3-(methylamino)butanenitrile (350 mg, 3.07 mmol, intermediate 15) in tetrahydrofuran (15 mL) was added 60% NaH (245 mg, 6.15 mmol) at 0° C. The resulting solution was stirred at room temperature for 0.5 h. Then 7-bromo-6-chloro-5,8-difluoroquinazolin-4-ol (600 mg, 2.03 mmol, intermediate 2) was added. Stirred at room temperature for 2 hours. The reaction was quenched with water. Concentrated under vacuum. The residue was purified by flash chromatography on silica gel (gradient: 0%-10% MeOH/DCM) to yield 430 mg (54.5% yield) of the title compound as a yellow solid. LC-MS: (ESI, m/z): [M+H]⁺=389.

Step 2: 2-(9-Bromo-8-chloro-10-fluoro-4-methyl-5,6-dihydro-4H-[1,4]oxazepino[5,6,7-de]quinazolin-5-yl)acetonitrile

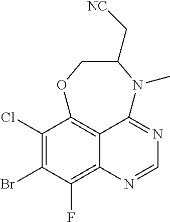 [SMILES: CN1c2ncnc3c(F)c(Br)c(Cl)c(c23)OCC1CC#N]

To a solution of 4-((7-bromo-6-chloro-8-fluoro-4-hydroxyquinazolin-5-yl)oxy)-3-(methylamino)butanenitrile (429 mg, 1.10 mmol) and DBU (670 mg, 4.41 mmol) in dichloromethane (25 mL) was added PyAOP (1.14 g, 2.19 mmol) at room temperature. The resulting solution was stirred at room temperature for 1 h. Concentrated under vacuum. The residue was purified by flash chromatography on silica gel (gradient: 0%-60% EtOAc/DCM) to yield 650 mg (95.3% yield) of the title compound as a yellow solid. LC-MS: (ESI, m/z): [M+H]⁺=371.

Step 3: 2-(9-(6-(bis(4-Methoxybenzyl)amino)-4-methylpyridin-2-yl)-8-chloro-10-fluoro-4-methyl-5,6-dihydro-4H-[1,4]oxazepino[5,6,7-de]quinazolin-5-yl)acetonitrile

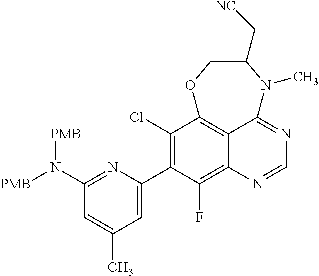

Under nitrogen, a solution of 2-(9-bromo-8-chloro-10-fluoro-4-methyl-5,6-dihydro-4H-[1,4]oxazepino[5,6,7-de]quinazolin-5-yl)acetonitrile (598 mg, 1.61 mmol), N,N-bis(4-methoxybenzyl)-4-methyl-6-(tributylstannyl)pyridin-2-amine (1.59 g, 2.49 mmol, intermediate 9), Pd(PPh₃)₄(374 mg, 0.320 mmol), CuI (30.8 mg, 0.160 mmol) and LiCl (169 mg, 4.05 mmol) in 1,4-dioxane (20 mL) was stirred at 110° C. for 1 h. The mixture was cooled to room temperature and the solid was filtered off, the filtrate was concentrated under reduced pressure. The residue was purified by flash chromatography on silica gel (gradient: 0%-60% EtOAc/DCM) to yield 598 mg (58.1% yield) of the title compound as a yellow solid. LC-MS: (ESI, m/z): [M+H]⁺=639.2.

Step 4: 2-(9-(6-(bis(4-Methoxybenzyl)amino)-3-iodo-4-methylpyridin-2-yl)-8-chloro-10-fluoro-4-methyl-5,6-dihydro-4H-[1,4]oxazepino[5,6,7-de]quinazolin-5-yl)acetonitrile

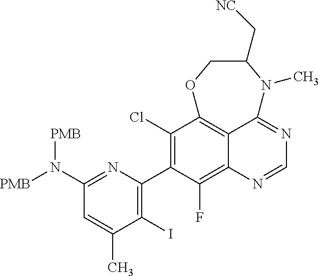

A solution of 2-(9-(6-(bis(4-methoxybenzyl)amino)-4-methylpyridin-2-yl)-8-chloro-10-fluoro-4-methyl-5,6-dihydro-4H-[1,4]oxazepino[5,6,7-de]quinazolin-5-yl)acetonitrile (300 mg, 0.470 mmol) and NIS (105 mg, 0.470 mmol) in acetic acid (4 mL) was stirred at room temperature for 1 hour. The reaction mixture was diluted with water and extracted with EtOAc. The combined organic layers were washed with aqueous Na₂S₂O₃, dried over anhydrous sodium sulfate and concentrated under vacuum. The residue was purified by flash chromatography on silica gel (gradient: 0%-30% EtOAc/DCM) to yield 220 mg (61.3% yield) of the title compound as a yellow solid. LC-MS: (ESI, m/z): [M+H]⁺=765.1.

Step 5: 2-(9-(6-(bis(4-Methoxybenzyl)amino)-4-methyl-3-(trifluoromethyl)pyridin-2-yl)-8-chloro-10-fluoro-4-methyl-5,6-dihydro-4H-[1,4]oxazepino[5,6,7-de]quinazolin-5-yl)acetonitrile

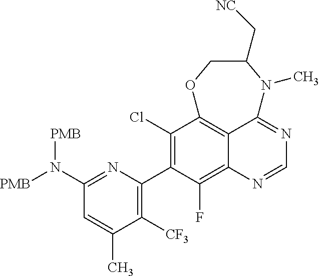

To a mixture of 2-(9-(6-(bis(4-methoxybenzyl)amino)-3-iodo-4-methylpyridin-2-yl)-8-chloro-10-fluoro-4-methyl-5,6-dihydro-4H-[1,4]oxazepino[5,6,7-de]quinazolin-5-yl)acetonitrile (200 mg, 0.260 mmol) and Cu (167 mg, 2.62 mmol) in N,N-dimethylformamide (5 mL) was added Cu(O₂CF₂SO₂F)₂ (1.09 g, 2.61 mmol) at 0° C. The resulting solution was stirred at 90° C. for 0.5 h. The reaction was cooled to room temperature, diluted with water and extracted with EtOAc. The combined organic layers were dried over anhydrous sodium sulfate and concentrated under vacuum. The residue was purified by flash chromatography on silica gel (gradient: 0%-40% EtOAc/DCM) to yield 135 mg (73% yield) of the title compound as a yellow oil. LC-MS: (ESI, m/z): [M+H]⁺=707.2.

Step 6: 2-(9-(6-Amino-4-methyl-3-(trifluoromethyl)pyridin-2-yl)-8-chloro-10-fluoro-4-methyl-5,6-dihydro-4H-[1,4]oxazepino[5,6,7-de]quinazolin-5-yl)acetonitrile(four unknown single isomers)

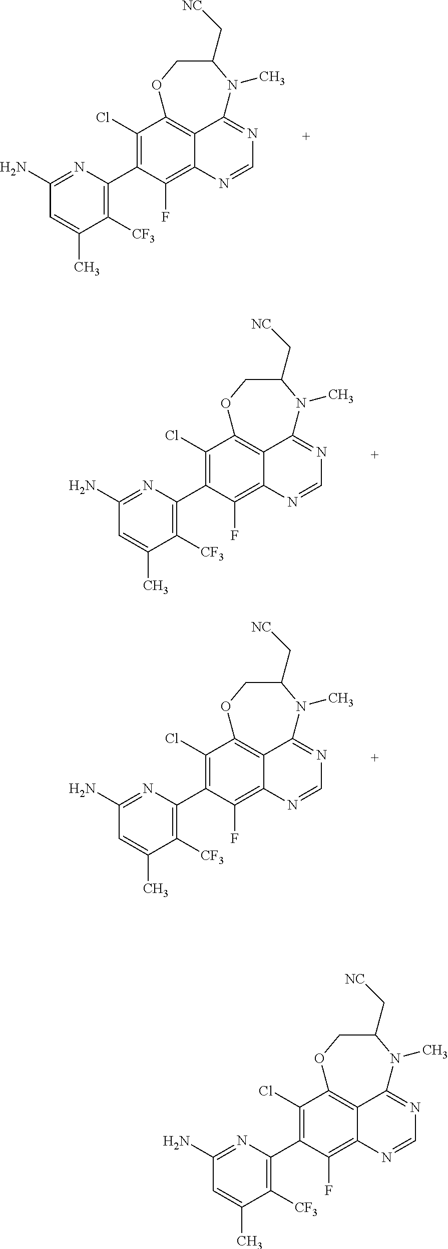 [SMILES: Cc1cc(N)nc(-c2c(Cl)c3c4c(ncnc4c2F)N(C)C(CC#N)CO3)c1C(F)(F)F ; Cc1cc(N)nc(-c2c(Cl)c3c4c(ncnc4c2F)N(C)C(CC#N)CO3)c1C(F)(F)F ; Cc1cc(N)nc(-c2c(Cl)c3c4c(ncnc4c2F)N(C)C(CC#N)CO3)c1C(F)(F)F ; Cc1cc(N)nc(-c2c(Cl)c3c4c(ncnc4c2F)N(C)C(CC#N)CO3)c1C(F)(F)F]

A solution of 2-(9-(6-(bis(4-methoxybenzyl)amino)-4-methyl-3-(trifluoromethyl)pyridin-2-yl)-8-chloro-10-fluoro-4-methyl-5,6-dihydro-4H-[1,4]oxazepino[5,6,7-de]quinazolin-5-yl)acetonitrile (130 mg, 0.180 mmol) in 2,2,2-trifluoroacetic acid (3 mL) was stirred at 50° C. for 5 hours. Concentrated under vacuum. The residue was purified by Prep-HPLC (Column: XBridge Prep C18 OBD Column, 30*100 mm, 5 μm; Mobile Phase A: Water (10 mmol/L NH4HCO3), Mobile Phase B: ACN; Flow rate: 60 mL/min; Gradient: 22% B to 45% B in 9 min, 45% B; Wave Length: 254/220 nm; RT1(min): 9.48; Number Of Runs: 0) to yield 37.0 mg (mixture of four isomers) of the title compound. The mixture was separated by Chiral-HPLC (Column: CHIRALPAK IE, 2*25 cm, 5 μm; Mobile Phase A: Hex:DCM=3:1(0.1% DEA)-HPLC, Mobile Phase B: EtOH-HPLC; Flow rate: 20 mL/min; Gradient: 10% B to 10% B in 14 min; Wave Length: 220/254 nm; RT1(min): 13.49; RT2(min): 23.50: RT3(min): 29.7: Sample Solvent: EtOH-HPLC; Injection Volume: 0.5 mL; Number Of Runs: 6) to yield 4.30 mg (5.00% yield) of Compound 525a (the third peak), 5.10 mg (5.90% yield) of Compound 525b (the fourth peak) and 16.0 mg (mixture of Compound 525c and Compound 525d). Then the 16 mg mixture was re-separated by Chiral-HPLC (Column: CHIRALPAK IG, 2*25 cm, 5 μm; Mobile Phase A: Hex (0.5% 2M NH3-MeOH)-HPLC, Mobile Phase B: EtOH-HPLC; Flow rate: 20 mL/min; Gradient: 20% B to 20% B in 14 min: Wave Length: 220/254 nm; RT1(min): 10.031; RT2(min): 11.68; Sample Solvent: EtOH-HPLC; Injection Volume: 0.5 mL; Number Of Runs: 6) to yield 4.50 mg (5.20% yield) of Compound 525c (the first peak) and 4.30 mg (5.00% yield) of Compound 525d (the second peak)

Compound 525a: LC-MS: (ESI, m/z): [M+H]⁺=467.1; ¹H NMR (400 MHz, DMSO-d₆, ppm) δ 8.57 (s, 1H), 6.87 (s, 2H), 6.49 (s, 1H), 4.80-4.71 (m, 1H), 4.58 (d, J=13.1 Hz, 1H), 4.47-4.38 (m, 1H), 3.39 (s, 3H), 3.25-3.16 (m, 1H), 3.12-3.02 (m, 1H), 2.38 (s, 3H)

Compound 525b: LC-MS: (ESI, m/z): [M+H]⁺=467.1; ¹H NMR (400 MHz, DMSO-d₆, ppm) δ 8.57 (s, 1H), 6.83 (s, 2H), 6.49 (s, 1H), 4.80-4.68 (m, 1H), 4.54 (d, J=13.2 Hz, 1H), 4.49-4.39 (m, 1H), 3.38 (s, 3H), 3.22-3.06 (m, 2H), 2.37 (s, 3H)

Compound 525c: LC-MS: (ESI, m/z): [M+H]⁺=467.1; ¹H NMR (300 MHz, DMSO-d₆, ppm) δ 8.58 (s, 1H), 6.89 (s, 2H), 6.50 (s, 1H), 4.83-4.69 (m, 1H), 4.60 (d, J=13.3 Hz, 1H), 4.49-4.38 (m, 1H), 3.40 (s, 3H), 3.29-3.15 (m, 1H), 3.13-3.01 (m, 1H), 2.39 (s, 3H)

Compound 525d: LC-MS: (ESI, m/z): [M+H]⁺=467.1; ¹H NMR (400 MHz, DMSO-d₆, ppm) δ 8.57 (s, 1H), 6.83 (s, 2H), 6.49 (s, 1H), 4.80-4.68 (m, 1H), 4.54 (d, J=13.2 Hz, 1H), 4.49-4.39 (m, 1H), 3.38 (s, 3H), 3.22-3.06 (m, 2H), 2.37 (s, 3H).

### Biological Assays

KRAS Biochemical Assay—BODIPY-GDP Exchange TR-FRET. Biochemical compound potencies were assessed by evaluating inhibition of SOS1-mediated nucleotide exchange in KRAS G12D. The SOS1-promoted exchange of fluorescently-labeled GDP (BODIPY-GDP) was monitored by time-resolved fluorescence resonance energy transfer (TR-FRET). Compounds solubilized in DMSO were dispensed as concentration series into 384-well white assay plates. A preformed complex of biotin-tagged recombinant human KRAS (1.5 nM mutant G12D or wild type) and 0.15 nM terbium-labeled streptavidin (CisBIO) prepared in 10 μL/well assay buffer (20 mM HEPES, pH 7.5, 50 mM NaCl, 10 mM MgCl₂, 0.01% Tween-20 and 1 mM dithiothreitol) was added and allowed to incubate for 10-minutes. The reaction was initiated with the addition of 5 μL of 3 nM recombinant human SOS1 and 300 nM BODIPY-GDP in assay buffer. After a 60-minute incubation, the fluorescence was measured with excitation at 337 nm and emission at 490 and 520 nm. The TR-FRET ratio was determined as the fluorescence at 520 nm divided by the fluorescence at 490 nm multiplied by 10,000. The results were normalized to percent inhibition based on control samples: DMSO (0% inhibition) and control compound at a concentration that inhibits completely (100% inhibition). The normalized TR-FRET results were plotted against compound concentration, and the data fit to a 4-parameter Hill equation to determine the IC₅₀ values.

KRAS 3D-Cell Proliferation Assays. Cellular potencies of compounds were assessed by evaluating inhibition of proliferation in 3D cultures of homozygous mutant KRAS G12D human pancreatic cell lines (AsPC-1 and SW1990) as compared to a KRAS wild type human lung adenocarcinoma cell line (PC-9). Cells were seeded into 384-well black round-bottom, ultra-low attachment assay plates in 50 μL cell growth medium (RPMI-1640 with 10% fetal bovine serum and 2 mM L-glutamine). After overnight incubation at 37° C. and 5% CO₂, compounds solubilized in DMSO were added as dilution series to the wells in a total volume of 150 nL (0.3% DMSO final). The cells were incubated for 7 days at 37° C. and 5% CO₂. Cell proliferation was quantitated by addition of 40 μL/well of CellTiter-Glo® 3D (Promega). This reagent in combination with mechanical disruption releases the cellular ATP to promote activity in a luciferase-based enzyme/substrate chemiluminescent detection system. After a 25-minute incubation under ambient conditions with shaking and an additional 10 minutes without shaking, the contents of the wells were mixed by pipetting up and down repeatedly to disrupt the spheroids, and then the plates were centrifuged to remove bubbles. The plates were incubated for another 15 minutes under ambient conditions, and the luminescence read on a plate reader (e.g. EnVision [PerkinElmer]). The results were normalized to percent inhibition based on the following control samples: DMSO (0% inhibition) and 1 uM staurosporine (100% inhibition). The normalized luminescence results were plotted against compound concentration, and the data fit to a 4-parameter Hill equation to determine the IC₅₀ values.

KRAS G12D NanoBRET Assay. Cellular target engagement was assessed by monitoring inhibition of the Raf-RBD/KRAS G12D interaction in a NanoBRET™ assay (Promega). The assay uses an HCT115 colon cancer cell line stably co-transfected with Raf-RBD fused to NanoLuc® luciferase and doxycycline-inducible KRAS G12D fused with a Halotag®. Cells were seeded into 384-well white tissue culture-treated assay plates in 40 μL culture medium (RPMI-1640 with 10% fetal bovine serum, 2 mM glutamine, 2 ng/mL puromycin, and 4 ng/mL blasticidin) with doxycycline to induce KRAS G12D-nanoluciferase expression over 20-24 h at 37° C., 5% CO₂. The culture medium was then removed and replaced with assay medium (Opti-MEM® with 4% fetal bovine serum) and 0.1 μM HaloTag618 ligand. During the subsequent 4 h incubation at 37° C., 5% CO₂, the HaloTag618 ligand binds to the Halotag-labeled KRAS G12D. Compounds solubilized in DMSO were then added as dilution series to the wells in a total volume of 160 nL (0.4% DMSO final), and the plates were incubated overnight at 37° C., 5% CO₂. In the final step, 10 μL NanoGlo substrate in Opti-MEM was added to each well, and the emission read at 460 nm (luciferase signal) and 610 nm (NanoBRET signal) on an EnVision plate reader (PerkinElmer). The Raf-RBD/KRAS G12D interaction results in bioluminescence resonance energy transfer (BRET) from the product of the luciferase reaction in the vicinity of the Raf-RBD to the HaloTag ligand acceptor on the KRAS G12D and generates the NanoBRET signal. Binding of compounds to KRAS G12D and disruption of its interaction with Raf-RBD results in reduction of this signal. The luciferase signal and NanoBRET signal for compound wells were divided by the average corresponding signal for DMSO control wells. Then the NanoBRET ratio was calculated by dividing the control-adjusted NanoBRET signal by the similarly adjusted luciferase signal. The results were normalized to percent inhibition based on control samples: DMSO (0% inhibition) and control compound at a concentration that inhibits completely (100% inhibition). The normalized NanoBRET ratio results were plotted against compound concentration, and the data fit to a 4-parameter Hill equation to determine the IC₅₀ values.

TABLE 3
activities of compounds (reported in uM)

| | KRas(G12V) | KRas GDP | SW480 Prolif | PC9 Prolif |
|---|---|---|---|---|
| | GDP HTRF | HTRF | IC50 | IC50 |
| Ex. No | (IC50) | (IC50) | (G12V) | (G12V) |
| 1 | 0.009 | 0.046 | 12 | 18 |
| 2 | 0.011 | 0.025 | 11 | 7.7 |
| 3 | 0.54 | 2.9 | ND | ND |
| 4 | 1.4 | 10 | ND | |
| 5 | 0.0015 | 0.0092 | 1.5 | 9 |
| 6 | 1.3 | 6.3 | ND | ND |
| 7 | 0.42 | 2.3 | ND | ND |
| 8 | 0.81 | 3.9 | ND | ND |
| 9 | 10 | 63 | ND | ND |
| 10 | 0.3 | 1.3 | ND | ND |
| 11 | 1 | 14 | ND | ND |
| 12 | 8.9 | 21 | ND | ND |
| 13 | 13 | 36 | ND | ND |
| 14 | 21 | 200 | ND | ND |
| 15 | 3.6 | 31 | ND | ND |
| 16 | 6.9 | 32 | ND | ND |
| 17 | 0.0061 | 0.052 | 8.7 | 17 |
| 18 | 0.035 | 0.2 | 20 | 20 |
| 19 | 15 | 200 | 5.7 | 4.5 |
| 20 | 0.069 | 0.51 | 7.3 | 8.7 |
| 21 | 1.6 | 3.4 | ND | ND |
| 22 | 11 | 43 | ND | ND |
| 23 | 0.0051 | 0.029 | 5.1 | 10 |
| 24 | 4.4 | 30 | ND | 20 |
| 25 | 0.51 | 2 | ND | ND |
| 26 | 0.053 | 0.33 | 9.7 | 16 |
| 27 | 0.58 | 1.4 | ND | ND |
| 28 | 0.0082 | 0.11 | 7.8 | 17 |
| 29 | 7.4 | 7.4 | ND | ND |
| 30 | 4.7 | 35 | ND | ND |
| 31 | 0.44 | 2.8 | ND | ND |
| 32 | ND | ND | ND | ND |
| 33 | ND | ND | ND | ND |
| 34 | 0.00087 | 0.0044 | 0.82 | 4 |
| 35 | 0.00039 | 0.00063 | 0.78 | 1.7 |
| 36 | 0.05 | 0.68 | 8.2 | 20 |
| 37 | 0.053 | 0.27 | ND | ND |
| 38 | ND | ND | ND | ND |
| 39 | 0.024 | 0.14 | 20 | 20 |
| 40 | 0.00099 | 0.0046 | 1.2 | 2.1 |
| 41a | 0.0018 | 0.011 | 1 | 3.8 |
| 41b | 1.2 | 6.5 | ND | ND |
| 42a | 0.0003 | 0.00025 | 0.085 | 1.3 |
| 42b | 0.055 | 0.082 | 3.4 | 7 |
| 43 | 2 | 4.9 | ND | ND |
| 44 | 0.0027 | 0.017 | 4.1 | 4.8 |
| 45 | 0.00023 | 0.00027 | 0.05 | 6 |
| 46 | 11 | 46 | ND | ND |
| 47a | 0.0011 | 0.0076 | 20 | 7.1 |
| 47b | 0.72 | 4.3 | ND | ND |
| 48 | 0.00004 | 0.000091 | 0.0014 | 1.5 |
| 49 | 0.00057 | 0.0038 | 1.1 | 3.8 |
| 50 | 0.002 | 0.014 | 0.93 | 6.7 |
| 51 | 9.5 | 12 | 20 | 20 |
| 52 | 2.4 | 9.6 | ND | ND |
| 53a | 0.00037 | 0.0012 | ND | ND |
| 53b | 0.6 | 2.9 | ND | ND |
| 54 | 0.041 | 0.31 | ND | ND |
| 55 | 0.019 | 0.13 | ND | NO |
| 56 | 0.022 | 0.16 | ND | ND |
| 57 | 0.27 | 2.0 | ND | ND |
| 58 | 0.018 | 0.034 | ND | ND |
| 59 | 0.057 | 0.27 | 7.1 | 5.8 |
| 60 | 0.00042 | 0.00048 | 0.23 | 2.7 |
| 61 | 0.00086 | 0.0043 | 0.94 | 3.1 |
| 62 | 0.012 | 0.096 | 4.9 | 3.4 |
| 63 | 0.0087 | 0.069 | 3.3 | 4 |
| 64 | 0.29 | 2.7 | 17 | 5.4 |
| 65a | 0.0001 | 0.00025 | 0.0026 | 0.94 |
| 66a | 0.00005 | 0.00012 | 0.0031 | 2.8 |
| 67 | 0.000170 | 0.00055 | 0.0037 | 2.8 |
| 68 | 0.0001 | 0.00031 | 0.011 | 1.8 |
| 69a | 0.00011 | 0.000295 | ND | ND |
| 70a | 0.00049 | 0.0083 | ND | ND |
| 71 | 0.000530 | 0.00661 | ND | ND |
| 72 | 0.012 | 0.143 | ND | ND |
| 73a | 0.00011 | 0.00027 | 0.0026 | 2.3 |
| 74a | 0.00009 | 0.00021 | 0.006 | 2.9 |
| 75a | 0.00005 | 0.000066 | 0.0011 | 0.89 |
| 76a | 0.00009 | 0.000102 | 0.0019 | ND |
| 77a | 0.00012 | 0.00033 | 0.021 | 20 |
| 77b | 0.00013 | 0.00034 | 0.065 | 20 |
| 78 | 0.00006 | 0.000209 | ND | ND |
| 79 | 0.00022 | 0.00034 | 0.0024 | 3.1 |
| 80 | 0.0082 | 0.0236 | 0.94 | 1.2 |
| 81 | 0.000067 | 0.0000659 | 0.0061 | 7.8 |
| 82a | 0.00011 | 0.000164 | 0.026 | 6.8 |
| 82b | 0.00012 | 0.000224 | 0.011 | 7.2 |
| 83a | 0.00019 | 0.000684 | 0.023 | 5.9 |
| 84a | 0.00014 | 0.000389 | 0.073 | 12 |
| 85 | 0.00009 | 0.000165 | ND | ND |
| 86 | 0.00015 | 0.00044 | 0.0049 | 3.7 |
| 87a | 0.000087 | 0.000106 | 0.0025 | 6.3 |
| 87b | 0.000089 | 0.00018 | 0.028 | 3.1 |
| 88 | 0.00005 | 0.00012 | 0.0015 | 3 |
| 89 | 0.000075 | 0.00017 | 0.0072 | 2.4 |
| 90 | 0.00022 | 0.00099 | 0.39 | 6.4 |
| 91a | 0.00029 | 0.0009 | 0.0034 | 20 |
| 91b | 0.00081 | 0.0022 | 0.14 | 6.4 |
| 92a | 0.00007 | 0.0000648 | 0.037 | 1.8 |
| 92b | 0.00031 | 0.00189 | 0.7 | 6 |
| 93a | 0.00009 | 0.000201 | ND | ND |
| 93b | 0.00014 | 0.00035 | ND | ND |
| 93c | 0.00007 | 0.000145 | ND | ND |
| 93d | 0.00007 | 0.0000849 | 0.0011 | ND |
| 94 | 0.00007 | 0.000175 | ND | ND |
| 95a | 0.00009 | 0.00083 | ND | ND |
| 95b | 0.00012 | 0.000484 | ND | ND |
| 96 | 0.00008 | 0.000299 | ND | ND |
| 97 | 0.00008 | 0.000359 | ND | ND |
| 98a | 0.00011 | 0.000557 | ND | ND |
| 98b | 0.0001 | 0.000125 | ND | ND |
| 99a | 0.00007 | 0.000125 | ND | ND |
| 99b | 0.00008 | 0.0000748 | ND | ND |
| 100a | 0.00003 | 0.00003 | ND | ND |
| 100b | 0.00073 | 0.000353 | ND | ND |
| 101 | 0.00006 | 0.0000616 | 0.0057 | 0.83 |
| 102a | 0.000094 | 0.000115 | 0.019 | 2.2 |
| 102b | 0.00017 | 0.00049 | 0.1 | 8.7 |
| 103a | ND | 0.00113 | ND | ND |
| 104a | 0.00015 | 0.000447 | 0.012 | ND |
| 104b | 0.016 | 0.0352 | ND | ND |
| 105a | 0.00008 | 0.00023 | 0.016 | 7.1 |
| 106 | 0.00013 | 0.00014 | 0.0094 | 0.97 |
| 107 | 0.09 | 0.24 | 0.56 | 4 |
| 108a | 0.00006 | 0.0002 | 0.002 | 1.4 |
| 109 | 0.00018 | 0.000222 | 0.0028 | 13 |
| 110a | 0.00033 | 0.0014 | 0.098 | 11 |
| 111a | 0.00036 | 0.00085 | 0.0018 | 7.4 |
| 112 | 0.0002 | 0.00032 | 0.0018 | 4.3 |
| 113a | 0.00009 | 0.0002 | 0.0028 | 7.5 |
| 114a | 0.00024 | 0.00039 | 0.0032 | 7 |
| 114b | 0.00022 | 0.00046 | 0.003 | 7.6 |
| 115 | 0.00047 | 0.0009 | 0.016 | 15 |
| 116 | 0.000074 | 0.0000519 | 0.0046 | 1.6 |
| 117 | 0.00008 | 0.00018 | 0.012 | 6.3 |
| 118 | 0.00006 | 0.00019 | 0.033 | 0.73 |
| 119 | 0.00005 | 0.00014 | 0.016 | 3 |
| 120a | 0.00007 | 0.0002 | 0.028 | 2.5 |
| 120b | 0.00008 | 0.0002 | 0.02 | 3 |
| 121 | 0.00007 | 0.000437 | 1.1 | 4.9 |
| 122a | 0.0005 | 0.00449 | 0.18 | 2.9 |
| 122b | 0.000095 | 0.000202 | 0.017 | 9.4 |
| 123a | 0.00013 | 0.00015 | 0.03 | 6.5 |
| 124 | 0.0001 | 0.000204 | 0.07 | 2.7 |
| 125a | 0.00012 | 0.000218 | 0.023 | 3.7 |
| 126 | 0.00034 | 0.00287 | ND | ND |
| 127 | 0.00007 | 0.0000894 | ND | ND |
| 128a | 0.00009 | 0.000149 | ND | ND |
| 128b | 0.00019 | 0.000469 | ND | ND |
| 129a | 0.00031 | 0.000232 | ND | ND |
| 129b | 0.00032 | 0.00039 | ND | ND |
| 130a | 0.00006 | 0.00007 | ND | ND |
| 131 | 0.00014 | 0.000253 | ND | ND |
| 132 | 0.00013 | 0.000273 | 0.039 | 3.2 |
| 133c | 0.00005 | 0.00015 | 0.025 | 7.1 |
| 134 | 0.00008 | 0.00021 | 0.59 | 1.6 |
| 135 | 0.00008 | 0.00037 | 0.39 | 1.5 |
| 136 | 0.00021 | 0.00083 | 0.063 | 8.6 |
| 137 | 0.00007 | 0.000159 | 0.14 | 1.1 |
| 138 | 0.00017 | 0.00058 | 0.077 | 4.9 |
| 139 | 0.00008 | 0.000205 | 0.24 | 0.83 |
| 140 | 0.00017 | 0.0006 | 0.051 | 3.8 |
| 141 | 0.00024 | 0.00077 | 0.076 | 8.1 |
| 142 | 0.00031 | 0.0011 | 0.12 | 7.7 |
| 143 | 0.00025 | 0.00087 | 0.094 | 10 |
| 144 | 0.0014 | 0.0044 | 0.52 | 6 |
| 145 | 0.00056 | 0.0012 | 0.45 | 20 |
| 146 | 0.00061 | 0.0015 | 0.51 | 19 |
| 147 | 0.00006 | 0.00018 | 0.044 | 1.3 |
| 148 | 0.0011 | 0.0036 | 0.41 | 9.6 |
| 149 | 0.00021 | 0.00078 | 0.074 | 16 |
| 150 | 0.0026 | 0.0082 | 0.79 | 8.9 |
| 151 | 0.0011 | 0.0054 | 0.7 | 14 |
| 152 | 0.00075 | 0.0023 | 0.18 | 5.6 |
| 153 | 0.00078 | 0.0033 | 0.19 | 14 |
| 154 | 0.00048 | 0.0011 | 0.091 | 14 |
| 155 | 0.00045 | 0.0025 | 0.22 | 10 |
| 156 | 0.00019 | 0.00065 | 0.31 | 16 |
| 157 | 0.00081 | 0.0032 | 0.4 | 8.2 |
| 158a | 0.00076 | 0.00541 | ND | ND |
| 158b | 0.097 | 0.68 | ND | ND |
| 159a | ND | ND | ND | ND |
| 159b | ND | ND | ND | ND |
| 160a | ND | ND | ND | ND |
| 160b | ND | ND | ND | ND |
| 161b | ND | ND | ND | ND |
| 162a | ND | ND | ND | ND |
| 162b | ND | ND | ND | ND |
| 162c | 0.37 | 0.83 | ND | ND |
| 162d | 0.41 | 0.896 | ND | ND |
| 163a | ND | ND | ND | ND |
| 163b | ND | ND | ND | ND |
| 164a | ND | ND | ND | ND |
| 164b | ND | ND | ND | ND |
| 165a | 0.00069 | 0.00214 | ND | ND |
| 165b | 0.89 | 2 | ND | ND |
| 166a | 0.00056 | 0.00206 | ND | ND |
| 166b | 0.64 | 1.73 | ND | ND |
| 167a | ND | ND | ND | ND |
| 167b | ND | ND | ND | ND |
| 169a | ND | ND | ND | ND |
| 169b | ND | ND | ND | ND |
| 170a | ND | ND | ND | ND |
| 170b | ND | ND | ND | ND |
| 171a | 0.0007 | 0.0022 | ND | ND |
| 171b | 0.9 | 2.09 | ND | ND |
| 172a | ND | ND | ND | ND |
| 172b | ND | ND | ND | ND |
| 173a | ND | ND | ND | ND |
| 173b | ND | ND | ND | ND |
| 174a | ND | ND | ND | ND |
| 174b | ND | ND | ND | ND |
| 175 | 0.00008 | 0.0001 | ND | ND |
| 176 | ND | ND | ND | ND |

*ND = Not Determined

TABLE 4
activities of compounds (reported in uM)

| | KRas | KRas | KRAS | | CTG |
|---|---|---|---|---|---|
| | (G12D) | (wt) | (G12D) | CTG | Panel |
| | GDP | GDP | HCT116 | Panel | Non |
| Cmpd | HTRF | HTRF | NanoBRET | (G12D) | G12D |
| No | (IC50) | (IC50) | (IC50) | IC50 | IC50 |
| 501 | 4.16 | 109 | 10 | ND | ND |
| 502 | 2.77 | 71.4 | 10 | 9.6455 | 7.88 |
| 503 | 4.62 | 94.3 | 10 | ND | ND |
| 504 | 3.46 | 15.1 | 10 | 14.8997 | 18.5 |
| 505 | 1.73 | 66.4 | 10 | ND | ND |
| 506 | 2.4 | 55 | 10 | ND | ND |
| 507 | 0.276 | 8.65 | 10 | ND | ND |
| 508 | 0.472 | 8.11 | 10 | ND | ND |
| 509 | 0.164 | 1.66 | 9.85 | ND | ND |
| 510 | 0.0136 | 0.24 | 10 | ND | ND |
| 511 | 0.00607 | 0.0484 | 8.054 | 2.2443 | 2.96 |
| 512 | 0.174 | 1.11 | ND | ND | ND |
| 513 | 0.00155 | 0.0108 | 2.577 | 1.0785 | 2.29 |
| 514 | 0.817 | 4.12 | 10 | ND | ND |
| 515 | 0.00339 | 0.02 | 7.983 | ND | ND |
| 516 | 0.0463 | 1.63 | 10 | ND | ND |
| 517 | 0.00579 | 0.00478 | 10 | ND | ND |
| 518 | 0.029 | 0.128 | 10 | ND | ND |
| 519 | ND | ND | ND | ND | ND |
| 520 | ND | ND | ND | ND | ND |
| 521 | ND | ND | ND | ND | ND |
| 522 | ND | ND | ND | NO | ND |
| 523 | 0.0119 | 0.088 | 10 | ND | 7.7 |
| 524 | 0.48 | 2.68 | 10 | ND | ND |
| 525 | ND | ND | ND | ND | ND |

*ND = not determined

All technical and scientific terms used herein have the same meaning. Efforts have been made to ensure accuracy with respect to numbers used (e.g. amounts, temperature, etc.) but some experimental errors and deviations should be accounted for.

Throughout this specification and the claims, the words “comprise,” “comprises,” and “comprising” are used in a non-exclusive sense, except where the context requires otherwise. It is understood that embodiments described herein include “consisting of” and/or “consisting essentially of” embodiments.

Where a range of values is provided, it is understood that each intervening value, to the tenth of the unit of the lower limit, unless the context clearly dictates otherwise, between the upper and lower limit of the range and any other stated or intervening value in that stated range, is encompassed herein. The upper and lower limits of these small ranges which can independently be included in the smaller rangers is also encompassed herein, subject to any specifically excluded limit in the stated range. Where the stated range includes one or both of the limits, ranges excluding either or both of those included limits are also included herein.

Many modifications and other embodiments of the compounds and methods set forth herein will come to mind to one skilled in the art having the benefit of the teachings presented in the foregoing descriptions and the associated drawings. Therefore, it is to be understood that the compounds and methods described herein are not to be limited to the specific embodiments disclosed and that modifications and other embodiments are intended to be included within the scope of the appended claims. The scope of the invention should, therefore, be determined not with reference to the above description, but should instead be determined with reference to the following appended claims, along with the full scope of equivalents to which such claims are entitled. Although specific terms are employed herein, they are used in a generic and descriptive sense only and not for purposes of limitation.

The patents, published applications, and scientific literature referred to herein establish the knowledge of those skilled in the art and are hereby incorporated by reference in their entirety to the same extent as if each was specifically and individually.

1. A compound having formula (I): 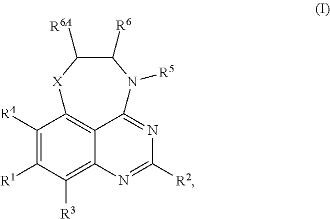
or a stereoisomer, atropisomer, tautomer, or pharmaceutically acceptable salt thereof, wherein:
X is NR¹³, O, C(R^{x})₂, C(O), SO, SO₂, or S;
each R^{x} is independently hydrogen, halogen, unsubstituted C₁₋₃ alkyl or unsubstituted C₁₋₃ haloalkyl;
or wherein two R^{x} together form a cyclopropyl together with the carbon to which they are bound;
R¹ is R⁷-substituted or unsubstituted indolyl, R⁷-substituted or unsubstituted benzofuranyl, R⁷-substituted or unsubstituted napthyl, R⁷-substituted or unsubstituted indazolyl, R⁷-substituted or unsubstituted indenyl, R⁷-substituted or unsubstituted benzothiazolyl, R^{7A}-substituted or unsubstituted phenyl, or R^{7A}-substituted or unsubstituted pyridinyl;
each R⁷ is independently hydrogen, halogen, CN, CH₂OH, —OH, NH₂, N(Me)₂, unsubstituted C₁₋₃ alkyl, unsubstituted C₂₋₅ alkynyl, unsubstituted C₁₋₃ haloalkyl, or unsubstituted cyclopropyl;
each R^{7A} is independently hydrogen, halogen, NH₂, N(Me)₂, unsubstituted C₁₋₃ alkyl, unsubstituted C₁₋₃ haloalkyl, or unsubstituted cyclopropyl;
R² is hydrogen, O-L¹-R⁸, R^{8A}-substituted or unsubstituted C₁₋₃ alkyl, or R^{8B}-substituted or unsubstituted 4-10 membered heterocycle;
L¹ is a bond or R^{L1}-substituted or unsubstituted C₁₋₃ alkylene;
R^{L1} is halogen or unsubstituted C₁₋₃ alkyl;
R⁸ is R⁹-substituted or unsubstituted 4-10 membered heterocycle comprising N, S, or O;
each R⁹ is independently halogen, oxo, unsubstituted C₁₋₃ alkyl, unsubstituted C₁₋₃ haloalkyl, unsubstituted C₁₋₃ alkoxy, R¹⁰-substituted or unsubstituted C₁₋₃ alkylidene, or R¹⁰-substituted or unsubstituted C₃₋₄ cycloalkyl, or R¹⁰-substituted or unsubstituted 3 or 4-membered heterocycle;
or wherein two R⁹ together form a C₃₋₅ cycloalkyl or 3-5 membered heterocycle;
R¹⁰ is hydrogen or halogen;
each R^{8A} is independently R^{9A}-substituted or unsubstituted C₁₋₃ alkyl, R^{9A}-substituted or unsubstituted C₁₋₃ alkoxy, R^{9A}-substituted or unsubstituted C34 cycloalkyl, or R^{9A}-substituted or unsubstituted 4-6 membered heterocycle;
each R^{9A} is independently halogen, oxo, unsubstituted C₁₋₃ alkyl, unsubstituted C₁₋₃ haloalkyl, unsubstituted C₁₋₃ alkoxy, unsubstituted C₁₋₃ alkylidene, R⁹-substituted or unsubstituted C₃₋₄ cycloalkyl, or R⁹-substituted or unsubstituted 4-10 membered heterocycle comprising N, S, or O;
R^{8B} is independently halogen, oxo, —NH₂, unsubstituted C₁₋₃ alkyl, unsubstituted C₁₋₃ haloalkyl, unsubstituted C₁₋₃ alkoxy, or unsubstituted C₁₋₃ alkylidene;
R³ and R⁴ are each independently hydrogen, —CN, halogen, unsubstituted C₁₋₃ alkyl, or unsubstituted cyclopropyl;
R⁵ is R^{5A}-substituted or unsubstituted C16 alkyl, R^{5A}-substituted or unsubstituted C1-haloalkyl, R^{5A}-substituted or unsubstituted C₃₋₁₀ cycloalkyl, R^{5A}-substituted or unsubstituted 3-10 membered heterocycle, or R^{SA}-substituted or unsubstituted 5-10 membered heteroaryl;
each R^{5A} is independently halogen, oxo, CN, OR¹¹, SR¹², SO₂R¹², NR¹³R¹⁴, C(O)N(R¹¹)₂, C(O)R¹¹, R^{5B}-substituted or unsubstituted C₁₋₆ alkyl, R^{5B}-substituted or unsubstituted C₁₋₆ haloalkyl, R^{5B}-substituted or unsubstituted C₃₋₄ cycloalkyl, R^{5B}-substituted or unsubstituted 3-6 membered heterocycle, R^{5B}-substituted or unsubstituted C₅₋₈ aryl, or R^{5B}-substituted or unsubstituted 5-9 membered heteroaryl;
or wherein two R^{5A} together form a C₃₋₆ cycloalkyl or 3-6 membered heterocycle;
each R^{5B} is independently halogen, oxo, CN, OR¹¹, NR¹³R¹⁴, SR¹², SO₂R¹², C(O)N(R¹¹)₂, C(O)R¹¹, R^{5C}-substituted or unsubstituted C₁₋₃ alkyl, R^{5C}-substituted or unsubstituted C₁₋₃ haloalkyl, R^{5C}-substituted or unsubstituted C₃₋₄ cycloalkyl, R^{5C}-substituted or unsubstituted 3-6 membered heterocycle, R^{5C}-substituted or unsubstituted phenyl, or R^{5C}-substituted or unsubstituted 5-6 membered heteroaryl;
or wherein two R^{5B} together form a C₃₋₄ cycloalkyl or 3-6 membered heterocycle;
each R^{5C} is independently halogen, oxo, CN, C(O)CH₃, C(O)NH₂, OH, OCH₃, CF₃, CHF₂, CH₂F, NR¹³R¹⁴, SCH₃, SO₂NH₂, SO₂CH₃, unsubstituted C₁₋₃ alkyl, unsubstituted C₁₋₃ haloalkyl, unsubstituted C₃₋₄ cycloalkyl, or unsubstituted 3-4 membered heterocycle;
each R¹¹ is independently hydrogen, unsubstituted C₁₋₃ alkyl, unsubstituted C₁₋₃ haloalkyl, unsubstituted C₃₋₄ cycloalkyl, or unsubstituted 3-4 membered heterocycle;
each R¹² is independently NH₂ or unsubstituted C₁₋₃ alkyl;
each R¹³ and R¹⁴ are independently hydrogen, C(O)R¹¹, C(O)N(R¹¹)₂, R¹⁵-substituted or unsubstituted C₁₋₆ alkyl, R¹⁵-substituted or unsubstituted C₃₋₆ cycloalkyl, or R¹⁵-substituted or unsubstituted 3-6 membered heterocycle;
each R¹⁵ is halogen, CN, C(O)CH₃, C(O)NH₂, OH, OCH₃, CF₃, CHF₂, CH₂F, NH₂, NHCH₃, N(CH₃)₂, SO₂NH₂, SO₂CH₃, R¹⁶-substituted or unsubstituted C₁₋₃ alkyl, R¹⁶-substituted or unsubstituted C₃₋₆ cycloalkyl, R¹⁶-substituted or unsubstituted 3-6 membered heterocycle, R⁶-substituted or unsubstituted 5-9 membered aryl, or R¹⁶-substituted or unsubstituted 5-9 membered heteroaryl;
each R¹⁶ is independently halogen, CN, C(O)CH₃, C(O)NH₂, OH, OCH₃, CF₃, CHF₂, CH₂F, NH₂, NHCH₃, N(CH₃)₂, SO₂NH₂, SO₂CH₃, R₁₇-substituted or unsubstituted C₁₋₃ alkyl, R¹⁷-substituted or unsubstituted C₃₋₆ cycloalkyl, R¹⁷-substituted or unsubstituted 3-6 membered heterocycle, R¹⁷-substituted or unsubstituted 5-9 membered aryl, or R¹⁷-substituted or unsubstituted 5-9 membered heteroaryl;
each R¹⁷ is independently halogen, CN, C(O)CH₃, C(O)NH₂, OH, OCH₃, CF₃, CHF₂, CH₂F, NH₂, NHCH₃, N(CH₃)₂, SO₂NH₂, SO₂CH₃, or unsubstituted C₁₋₃ alkyl;
R⁶ and R^{6A} are independently hydrogen, halogen, NR¹³R¹⁴, or R^{6B}-substituted or unsubstituted C₁₋₆ alkyl; and
R^{6B} is halogen, CN, OH, OCH₃, CF₃, CHF₂, CH₂F, or unsubstituted C₁₋₃ alkyl.

2. The compound of claim 1, wherein R¹ is R^{7A}-substituted or unsubstituted phenyl, R⁷-substituted or unsubstituted indazolyl, or R^{7A}-substituted or unsubstituted pyridinyl.

3. (canceled)

4. (canceled)

5. (canceled)

6. The compound of claim 1, wherein each R^{7A} is independently halogen, NH₂, unsubstituted C₁₋₃ alkyl, or unsubstituted C₁₋₃ haloalkyl.

7. The compound of claim 1, wherein;
R¹ is 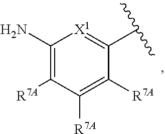
wherein:
X¹ is N, CH, or CF; and
R^{7A} is hydrogen, halogen, unsubstituted C₁₋₃ alkyl, or unsubstituted C₁₋₃ haloalkyl.

8. The compound of claim 1, wherein R¹ is

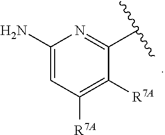

9. The compound of claim 1, wherein R¹ is

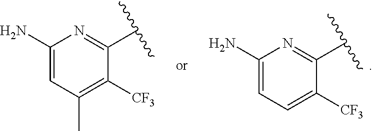 [SMILES: *c1nc(N)cc(C)c1C(F)(F)F ; *c1nc(N)ccc1C(F)(F)F]

10. The compound of claim 1, wherein R¹ is 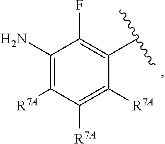
wherein R^{7A} is hydrogen, halogen, unsubstituted C₁₋₃ alkyl or unsubstituted C₁₋₃ haloalkyl.

11. The compound of claim 1, wherein R¹ is

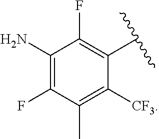 [SMILES: *c1c(F)c(N)c(F)c(C)c1C(F)(F)F]

12. The compound of claim 1, wherein R¹ is 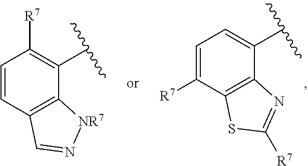
wherein each R⁷ is independently halogen, NH₂, N(Me)₂, unsubstituted C₁₋₃ alkyl, or unsubstituted C₁₋₃ haloalkyl.

13. The compound of claim 1, wherein R² is O-L¹-R⁸, R^{8A}-substituted or unsubstituted C₁₋₃ alkyl, or R^{8B}-substituted or unsubstituted 4-6 membered heterocycle.

14. The compound of claim 1, wherein R² is O-L¹-R⁸ and L¹ is unsubstituted C₁₋₃ alkylene.

15. (canceled)

16. The compound of claim 1, wherein R⁸ is a 4-10 membered heterocycle comprising one N.

17. The compound of claim 1, wherein R⁸ is 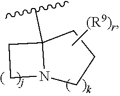
wherein:
R⁹ is halogen or R¹⁰-substituted or unsubstituted C₁₋₃ alkylidene;
r is an integer from 0-12;
j is 1, 2, or 3; and
k is 1 or 2.

18. The compound of claim 0, wherein r is 0, 1, 2, or 3.

19. The compound of claim 17, wherein R⁸ is 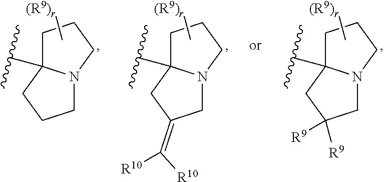
wherein:
R⁹ is independently halogen or R¹⁰-substituted or unsubstituted C₁₋₃ alkylidene;
each R¹⁰ is independently hydrogen or halogen; and
r is 1 or 2.

20. The compound of claim 1, wherein R⁸ is 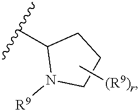
wherein:
R⁹ is independently halogen, oxo, or unsubstituted C₁₋₃ alkyl;
or wherein two R⁹ together form a C₃₋₅ cycloalkyl or 3-5 membered heterocycle; and
r is 1 or 2.

21. The compound of claim 1, wherein R⁸ is 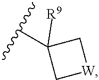
wherein:
R⁹ is hydrogen or unsubstituted C₁₋₃ alkyl;
W is O, SO₂, or NR¹²; and
R¹² is hydrogen, unsubstituted C₁₋₃ alkyl, or unsubstituted C₁₋₃ haloalkyl.

22. The compound of claim 21, wherein R⁸ is azetidinyl, oxetanyl, or thietanedioxide.

23. The compound claim 1, wherein R² is

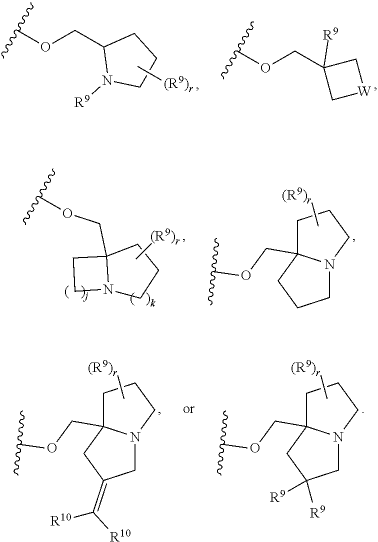

24. The compound of claim 0, wherein R⁹ is halogen or R¹⁰-substituted or unsubstituted C₁₋₃-alkylidene.

25. The compound of claim 1, wherein R² is hydrogen.

26. The compound of claim 1, wherein R³ is hydrogen or halogen.

27. The compound of claim 1, wherein R⁴ is halogen.

28. The compound of claim 1, wherein R⁵ is R^{5A}-substituted or unsubstituted C₁₋₆ alkyl.

29. The compound of claim 1, wherein R⁵ is

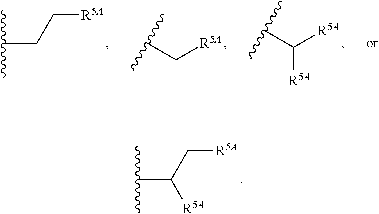

30. The compound of claim 1, wherein R⁵ is 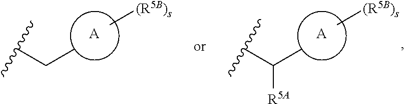
wherein:
ring A is a 3-6 membered heterocycle or 5-9 membered heteroaryl comprising at least one N; and
s is 0, 1, 2, or 3.

31. The compound of claim 0, wherein ring A is azetidinyl, thietanyl 1,1-dioxide, imidazolyl, thiazolyl, isothiazolyl, triazolyl, pyrazolyl, pyrazinyl, pyridonyl, pyridinyl, pyrimidinyl, pyridazinyl, pyrrolopyridinyl, or pyrazolopyridinyl.

32. The compound of claim 0, wherein ring A is imidazolyl, isothiazolyl, or triazolyl.

33. The compound of claim 0, wherein ring A is pyrazolyl, pyridonyl, pyridinyl, pyrimidinyl, or pyridazinyl.

34. The compound of claim 0 wherein R⁵ is:

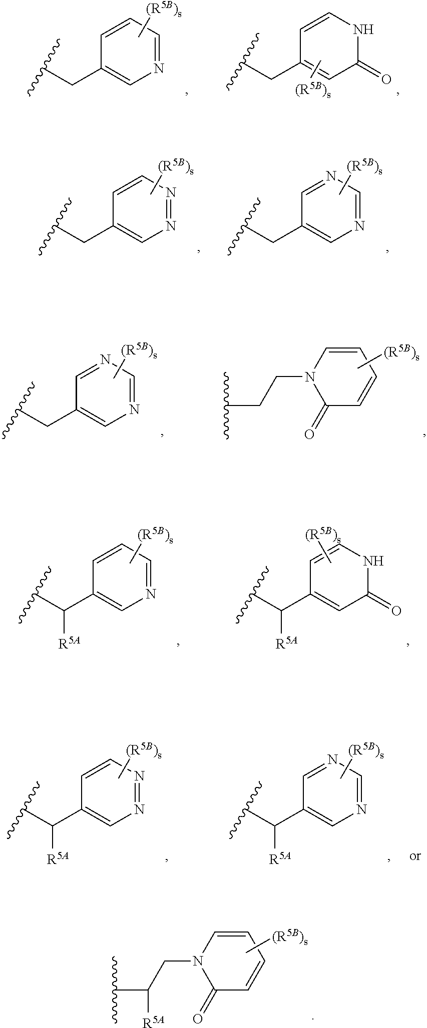

35. (canceled)

36. The compound of claim 1, wherein R⁵ is 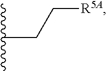
wherein:
R^{5A} is CN, OH, COR¹¹, SO₂R¹², NR¹³R¹⁴, R^{5B}-substituted or unsubstituted azetidinyl, or R^{5B}-substituted or unsubstituted oxetanyl.

37. The compound of claim 1, wherein R⁵ is R^{5A}-substituted or unsubstituted 5-9 membered heteroaryl.

38. (canceled)

39. The compound of claim 1 having a formula selected from: 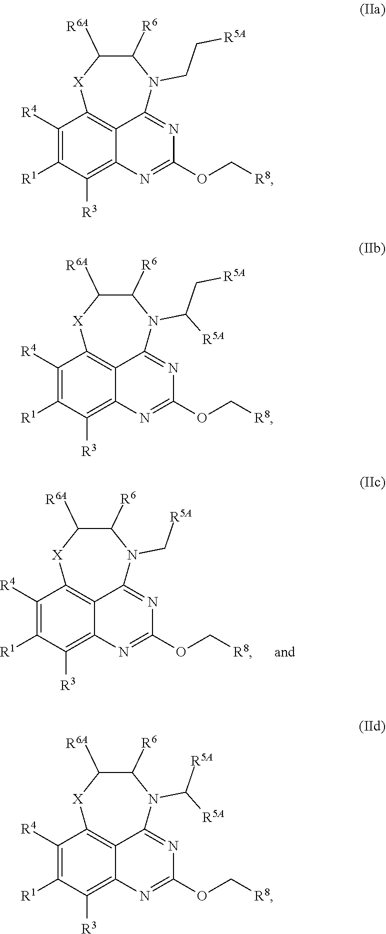
or a stereoisomer, atropisomer, tautomer, or pharmaceutically acceptable salt thereof.

40. The compound of claim 1 having a formula selected from: 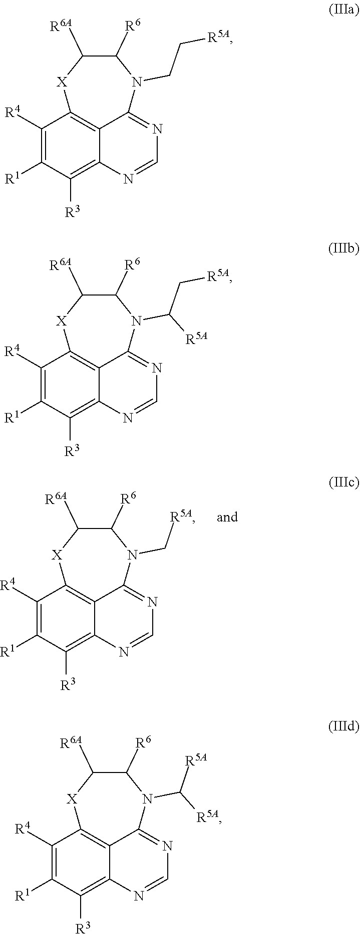
or a stereoisomer, atropisomer, tautomer, or pharmaceutically acceptable salt thereof.

41. The compound of claim 1 having a formula selected from: 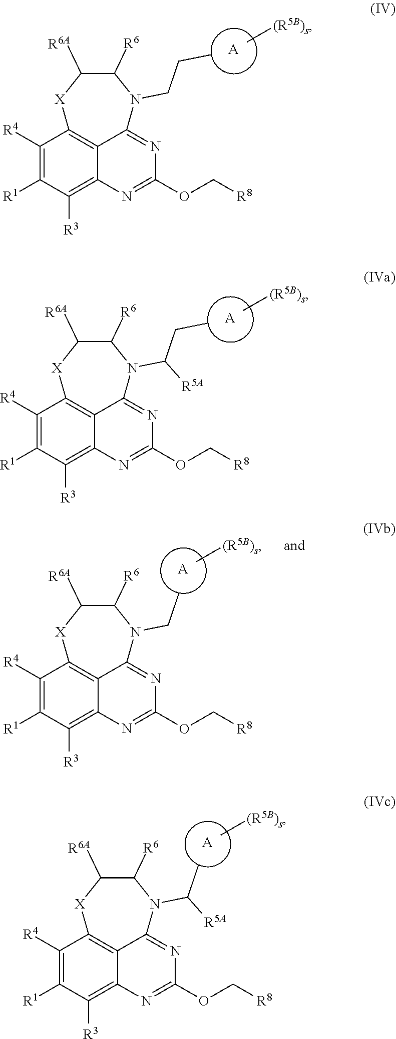
wherein ring A is a 3-6 membered heterocycle or 5-9 membered heteroaryl comprising at least one N; and
s is 0, 1, 2, or 3;
or a stereoisomer, atropisomer, tautomer, or pharmaceutically acceptable salt thereof.

42. The compound of claim 1 having a formula selected from: 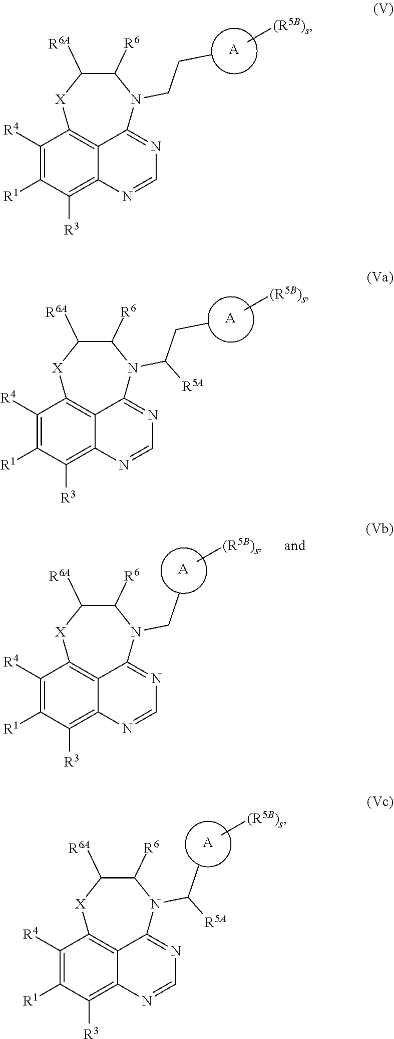
wherein ring A is a 3-6 membered heterocycle or 5-9 membered heteroaryl comprising at least one N; and
s is 0, 1, 2, or 3:
or a stereoisomer, atropisomer, tautomer, or pharmaceutically acceptable salt thereof.

43. (canceled)

44. (canceled)

45. The compound of claim 1, wherein R⁸ is:

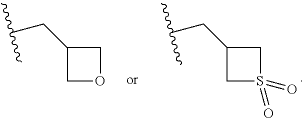 [SMILES: *CC1COC1 ; "or" ; *CC1CS(=O)(=O)C1 ; "."]

46. The compound of claim 1, wherein X is O.

47. The compound of claim 1, wherein X is C(R^{x})₂.

48. The compound claim 1, wherein R⁶ is R^{6A}-substituted or unsubstituted C₁₋₃ alkyl.

49. The compound claim 1, wherein R⁶ is R^{6A}-substituted C₁₋₃ alkyl.

50. The compound of claim 0, wherein R^{6A} is halogen, CN, or OH.

51. The compound of claim 1, wherein R⁶ is hydrogen.

52. A compound of Table 1 or a stereoisomer, atropisomer, tautomer, or pharmaceutically acceptable salt thereof.

53. A compound of Table 2 or a stereoisomer, atropisomer, tautomer, or pharmaceutically acceptable salt thereof.

54. A pharmaceutical composition comprising a compound or a stereoisomer, atropisomer, tautomer, or pharmaceutically acceptable salt thereof of claim 1 and one or more pharmaceutically acceptable excipients.

55. A method of treating a cancer comprising a KRas mutation, the method comprising:
administering to a patient in need thereof an effective amount of a compound or a stereoisomer, atropisomer, tautomer, or pharmaceutically acceptable salt thereof of claim 1.

56. (canceled)

57. The method of claim 5, wherein the KRas mutation is a KRas^{G12D} mutation or a KRas^{G12V} mutation.

58. The method of claim 5, further comprising testing a sample from the patient before administration for the absence or presence of a KRas mutation.

59. The method of claim 0, wherein the compound, stereoisomer, atropisomer, tautomer, or pharmaceutically acceptable salt thereof or pharmaceutical composition is administered to the patient after the patient sample shows the presence of the KRas mutation.

60. The method of claim 55, wherein the cancer is tissue agnostic.

61. The method of claim 55, wherein the cancer is pancreatic cancer, lung cancer, or colorectal cancer.

62. The method of claim 0, wherein the lung cancer is lung adenocarcinoma, NSCLC, or SCLC.

63. (canceled)

64. (canceled)

65. The method of claim 55, further comprising administering at least one additional therapeutic agent.

66. The method of claim 0, wherein the additional therapeutic agent comprises an epidermal growth factor receptor (EGFR) inhibitor, phosphatidylinositol kinase (PI3K) inhibitor, insulin-like growth factor receptor (IGF1R) inhibitor, a Janus kinase (JAK) inhibitor, a Met kinase inhibitor, a SRC family kinase inhibitor, a mitogen-activated protein kinase (MEK) inhibitor, an extracellular-signal-regulated kinase (ERK) inhibitor, a topoisomerase inhibitor, a taxane, an anti-metabolite agent, or an alkylating agent.

67. (canceled)

68. (canceled)

69. (canceled)

70. (canceled)

71. (canceled)

72. A method for regulating activity of a KRas mutant protein, the method comprising reacting the KRas mutant protein with a compound of claim 1, or a stereoisomer, atropisomer, tautomer, or pharmaceutically acceptable salt thereof.

73. A method for inhibiting proliferation of a cell population, the method comprising contacting the cell population with the compound of claim 1, or a stereoisomer, atropisomer, tautomer, or pharmaceutically acceptable salt thereof.

74. The method of claim 0, wherein the inhibition of proliferation is measured as a decrease in cell viability of the cell population.

75. (canceled)

76. A method for inhibiting tumor metastasis, the method comprising administering to an individual in need thereof a therapeutically effective amount of the compound of claim 1, or a stereoisomer, atropisomer, tautomer, or pharmaceutically acceptable salt thereof.

77. (canceled)
